(19) United States
(12) Patent Application Publication
(10) Pub. No.: US 2025/0163038 A1
(43) Pub. Date: May 22, 2025

(54) CBL-B MODULATORS AND USES THEREOF

(71) Applicant: Nimbus Clio, Inc., Bostoon, MA (US)

(72) Inventors: Silvana Marcel LEIT DE MORADEI, Burlington, MA (US); Angela V. WEST, Franklin, MA (US); Thomas BAKER, Nottingham (GB); Jokin Carrillo ARREGUI, Nottingham (GB); Diana CASTAGNA, Nottingham (GB); Jeremy Robert GREENWOOD, Brooklyn, NY (US); Salma RAFI, Lexington, MA (US); Fiona MCROBB, New York, NY (US); Yan ZHANG, Jersey City, NJ (US)

(21) Appl. No.: 18/573,342

(22) PCT Filed: Jun. 21, 2022
(86) PCT No.: PCT/US2022/073060

§ 371 Date: Dec. 21, 2023

Related U.S. Application Data
(60) Provisional application No. 63/363,133, filed on Apr. 18, 2022.
(60) Provisional application No. 63/202,693, filed on Jun. 21, 2021.

(51) Int. Cl. C07D 413/14; A61K 31/4196; A61K 31/423; A61K 31/433; A61K 31/438; A61K 31/4439; A61K 31/496; A61K 31/5377; C07D 401/10; C07D 403/10; C07D 403/14; C07D 405/14; C07D 413/10; C07D 417/14

(52) U.S. Cl. CPC C07D 413/14; A61K 31/4196; A61K 31/423; A61K 31/433; A61K 31/438; A61K 31/4439; A61K 31/496; A61K 31/5377; C07D 401/10; C07D 403/10; C07D 403/14; C07D 405/14; C07D 413/10; C07D 417/14

(57) ABSTRACT

The invention provides compounds, compositions thereof, and methods of using the same for the inhibition of Cbl-b, and the treatment of a variety of Cbl-b-mediated diseases, disorders or conditions, associated with modulating the immune system implicating Cbl-b. Compounds are also useful for the study of Cbl-b enzymes in biological and pathological phenomena; the study of ubiquitination occurring in bodily tissues; and the comparative evaluation of new Cbl-b inhibitors or other regulators of cell cycle, DNA repair, differentiation, and innate and adaptive immunity in vitro or in vivo.

### CROSS-REFERENCE TO RELATED APPLICATIONS

This application claims the benefit of priority to U.S. Provisional Appl. No. 63/202,693, filed Jun. 21, 2021, and U.S. Provisional Appl. No. 63/363,133, filed Apr. 18, 2022, the entirety of each of which is herein incorporated by reference.

### TECHNICAL FIELD OF THE INVENTION

The present invention relates to compounds and methods useful for inhibiting E3 ligase Casitas B-lineage lymphoma b (Cbl-b). The invention also provides pharmaceutically acceptable compositions comprising compounds of the present invention and methods of using said compositions in the treatment of various disorders.

### BACKGROUND OF THE INVENTION

Ubiquitination is a post-translational modification that regulates the function and fate of proteins involved with physiological processes. The addition of ubiquitin to target proteins occurs via a three-step enzymatic process that involves three enzymes. The first enzyme, E1, catalyzes ubiquitin activation. Activated ubiquitin is then transferred from E1 to the ubiquitin-conjugating enzyme, E2. The third enzyme, or E3 ligase, confers substrate specificity and directly catalyzes the transfer of ubiquitin from the E2 into the protein substrate. The addition of poly-ubiquitin chains to proteins serves as a signal leading to degradation into peptides of the ubiquitin-conjugated protein by the proteasome. Additionally, poly- and mono-ubiquitination can also alter cellular localization, function, and interactions of the protein substrate with proteins required for downstream activity and signaling events.

Ubiquitination controls multiple biological processes that are often dysregulated in disease, including cell cycle, DNA repair, differentiation, and innate and adaptive immunity. Therefore, the discovery of molecules that modulate components of the ubiquitin proteasome system represents an attractive therapeutic opportunity for a wide range of conditions, including cancer and auto-immune disease.

The compounds and compositions described herein are generally useful for the inhibition of the E3 ligase Casitas B-lineage lymphoma b (Cbl-b).

Cbl-b is a RING finger E3 ligase and a member of a highly conserved family of Cbl proteins, which in mammals consists of three Cbl genes: Cbl, Cbl-b, and Cbl-c. Cbl proteins interact with target proteins via their protein-protein interaction domains, allowing regulation of multiple signaling pathways, including tyrosine kinase (TK) signaling in multiple cell types. The structure of Cbl proteins consists of an amino-terminal tyrosine kinase binding domain (TKBD), a linker helix region (LHR) and a really interesting new gene (RING) domain, followed by a carboxy-terminal region containing binding sites for Src homology 2 (SH2) and Src homology 3 (SH3) domains. Cbl TKBD is composed of a four-helix bundle (4H), an EF-hand, and a variant SH2 domain, which binds substrates, such as activated TKs, in a phospho-tyrosine dependent manner.

Ubiquitination of activated receptor TKs by Cbl-b regulates the assembly of endocytic proteins both at the membrane and at sorting endosomes to promote lysosome targeting, degradation and signal termination. Cbl-b is also important for down regulation of signaling from antigen and cytokine receptors through ubiquitination of receptor chains and associated cytosolic TKs, leading to inactivation and/or proteasomal degradation.

Cbl-b is expressed in immune cell lineages and acts as a major regulator of immune cell activation and maintenance of peripheral tolerance. Cbl-b negatively regulates adaptive immune system signaling by establishing the threshold for the activation of antigen receptors. In T cells, Cbl-b imposes a requirement for a co-stimulatory signal to mount a productive immune response upon T cell receptor (TCR) engagement. Mice deficient in Cbl-b, and more specifically in the RING Zn-finger ligase domain of Cbl-b, showed tumor rejection that is mediated by CD8+ T cells.

Additionally, Cbl-b regulates the activity of multiple cell lineages involved in innate immunity, including NK cells, antigen-presenting dendritic cells (DC) and monocytes. Therefore, due to the complexity and diversity of the protein targets of Cbl-b in a variety of immune cells, it is possible that the functions of Cbl-b are cell-type dependent.

Novel therapeutic approaches aimed at removing inhibitory signals in immune cells to boost a productive immune system have gained recent attention. Given the central role that Cbl-b plays in regulating multiples signaling mechanisms in both innate and adaptive immunity, inhibition of Cbl-b provides therapeutic opportunities, including cancer immunotherapies.

Cbl-b inhibitors may strengthen the activity of cancer vaccines. For example, it was reported that the adoptive transfer of Cbl-b−/− CD8+ T cells combined with DC vaccines delays tumor growth. Additionally, Cbl-b−/− T cells are resistant to inhibition by PDL-1/PD-1 in vitro and in vivo, which supports the rationale combination of Cbl-b inhibitors with anti-PD-1/PD-L1 checkpoint blockade.

Enhanced expression of Cbl-b associates with better prognosis in lung adenocarcinoma. Moreover, mutations in the RING finger domain of Cbl proteins and Cbl-b linker sequence are found in a variety of disorders and cancers, including Juvenile myelomonocytic leukemia (JMML), preleukemic chronic myelomonocytic leukemia (CMML), Myeloproliferative Neoplasms (MPN), and Acute myeloid leukemia (AML). These observations suggest that that degradation impairment of activated TKs represents an important cancer mechanism that involves Cbl proteins. In agreement, multiple reports have demonstrated the ubiquitination of the epidermal growth factor receptor (EGFR) and the platelet derived growth factor receptor alpha (PDGFRa) by Cbl-b. The ubiquitination of these receptors promotes their proteasomal-dependent degradation in a variety of cancer lineages. The degradation of EGFR by Cbl-b leads to lung and gastric cancer cell proliferation and mediates epithelial to mesenchymal transition (EMT) in metastatic breast and gastric cancers. Additionally, amplifications and mutations of both EGFR and PDGFR are major drivers of oncogenic transformation and are commonly found in multiple types of cancer. Thus, Cbl-b inhibition represents an opportunity for both tumor intrinsic and tumor extrinsic therapies.

### SUMMARY OF THE INVENTION

It has now been found that compounds of this invention, and pharmaceutically acceptable compositions thereof, are effective as inhibitors of Cbl-b. In certain embodiments, the invention provides for compounds of the formulae presented herein.

Compounds of the present invention, and pharmaceutically acceptable compositions thereof, are useful for treating a variety of diseases, disorders or conditions, associated with modulating the immune system implicating Cbl-b. Such diseases, disorders, or conditions include those described herein.

Compounds provided by this invention are also useful for the study of Cbl-b enzymes in biological and pathological phenomena; the study of ubiquitination occurring in bodily tissues; and the comparative evaluation of new Cbl-b inhibitors or other regulators of cell cycle, DNA repair, differentiation, and innate and adaptive immunity in vitro or in vivo.

### DETAILED DESCRIPTION OF CERTAIN EMBODIMENTS

### 1. General Description of Certain Embodiments of the Invention

In certain aspects, the present invention provides a compound of formula I.

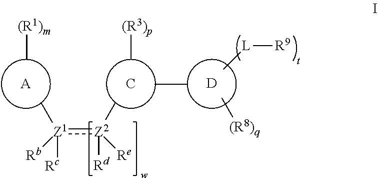

or a pharmaceutically acceptable salt thereof, wherein each of Ring A, Ring C, Ring D, L, R¹, R³, R⁸, R⁹, Z¹, Z², R^{b}, R^{c}, R^{d}, R^{e}, p, q, t, and w, is as defined below and described in embodiments herein, both singly and in combination.

In some embodiments, the present invention provides a pharmaceutical composition comprising a compound of formula I and a pharmaceutically acceptable carrier, adjuvant, or diluent.

In some embodiments, the present invention provides a method of treating a Cbl-b-mediated disease, disorder, or condition comprising administering to a patient in need thereof, a compound of formula I, or a pharmaceutically acceptable salt thereof.

### 2. Compounds and Definitions

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

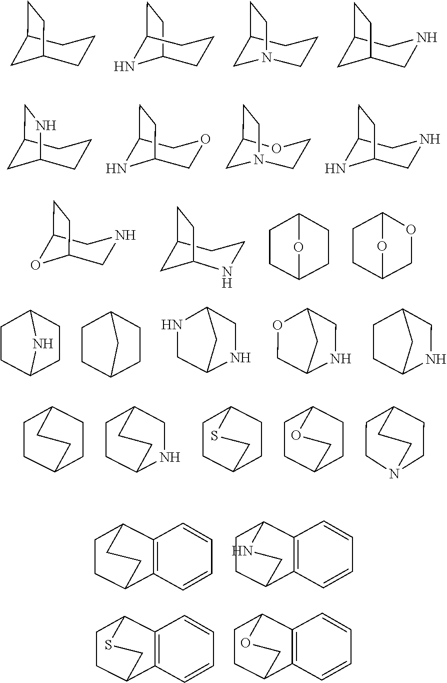

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where unless otherwise specified, the radical or point of attachment is on the heteroaromatic ring or on one of the rings to which the heteroaromatic ring is fused. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, 2-oxa-6-azaspiro[3.3]heptane, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —N(R^{∘})C(NR^{∘})N(R^{∘})₂; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH2)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; —SiR^{∘}₃; —(C₁₋₄ straight or branched alkylene)O—N(R^{∘})₂; or —(C₁₋₄ straight or branched alkylene)C(O)O—N(R^{∘})₂, wherein each R^{◯} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{◯} (or the ring formed by taking two independent occurrences of R^{◯} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{◯} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂)₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention. In certain embodiments, a warhead moiety, R¹, of a provided compound comprises one or more deuterium atoms. In certain embodiments, Ring B of a provided compound may be substituted with one or more deuterium atoms.

The structures as drawn represent relative configurations, unless labeled as absolute configurations. The invention contemplates individual enantiomers and racemic mixtures.

As used herein, a “Cbl-b inhibitor” is a molecule that reduces, inhibits, or otherwise diminishes one or more of the biological activities of Cbl-b (e.g., ubiquitination, regulation of tyrosine kinase signaling, or regulation of immune cell activation and maintenance of peripheral tolerance). Inhibition using the Cbl-b inhibitor does not necessarily indicate a total elimination of the Cbl-b activity. Instead, the activity could decrease by a statistically significant amount including, for example, a decrease of at least about 5%, 10%, 15%, 20%, 25%, 30%, 35%, 40%, 45%, 50%, 55%, 60%, 65%, 70%, 75%, 80%, 85%, 95% or 100% of the activity of Cbl-b compared to an appropriate control. In some embodiments, the Cbl-b inhibitor reduces, inhibits, or otherwise diminishes the ubiquitination activity of Cbl-b. In some of these embodiments, the Cbl-b inhibitor reduces, inhibits, or otherwise diminishes the Cbl-b-mediated ubiquitination of tyrosine kinases. The presently disclosed compounds bind directly to CBl-b and inhibit its ubiquitinating activity.

By “specific inhibitor” is intended an agent that reduces, inhibits, or otherwise diminishes the activity of a defined target greater than that of an unrelated target. For example, a Cbl-b specific inhibitor reduces at least one biological activity of Cbl-b by an amount that is statistically greater than the inhibitory effect of the antagonist on any other protein (e.g., other E3 ligases). In some embodiments, the IC₅₀ of the inhibitor for the target is about 90%, 80%, 70%, 60%, 50%, 40%, 30%, 20%, 10%, 5%, 1%, 0.1%, 0.01%, 0.001% or less of the IC₅₀ of the antagonist for a non-target. The presently disclosed compounds may or may not be a specific Cbl-b inhibitor. A specific Cbl-b inhibitor reduces the biological activity of Cbl-b by an amount that is statistically greater than the inhibitory effect of the antagonist on any other protein (e.g., other E3 ligases). In certain embodiments, the Cbl-b inhibitor specifically inhibits the ubiquitinating activity of Cbl-b. In some of these embodiments, the IC₅₀ of the Cbl-b inhibitor for Cbl-b is about 90%, 80%, 70%, 60%, 50%, 40%, 30%, 20%, 10%, 0.1%, 0.01%, 0.001%, or less of the IC₅₀ of the Cbl-b inhibitor for another RING finger E3 ligase or other type of E3 ligase (e.g., Cullin-RING ligase).

A compound of the present invention may be tethered to a detectable moiety. It will be appreciated that such compounds are useful as imaging agents. One of ordinary skill in the art will recognize that a detectable moiety may be attached to a provided compound via a suitable substituent. As used herein, the term “suitable substituent” refers to a moiety that is capable of covalent attachment to a detectable moiety. Such moieties are well known to one of ordinary skill in the art and include groups containing, e.g., a carboxylate moiety, an amino moiety, a thiol moiety, or a hydroxyl moiety, to name but a few. It will be appreciated that such moieties may be directly attached to a provided compound or via a tethering group, such as a bivalent saturated or unsaturated hydrocarbon chain. In some embodiments, such moieties may be attached via click chemistry. In some embodiments, such moieties may be attached via a 1,3-cycloaddition of an azide with an alkyne, optionally in the presence of a copper catalyst. Methods of using click chemistry are known in the art and include those described by Rostovtsev et al., Angew. Chem. Int. Ed. 2002, 41, 2596-99 and Sun et al., Bioconjugate Chem., 2006, 17, 52-57.

As used herein, the term “detectable moiety” is used interchangeably with the term “label” and relates to any moiety capable of being detected, e.g., primary labels and secondary labels. Primary labels, such as radioisotopes (e.g., tritium, ³²p ³³P, ³⁵S, or ¹⁴C), mass-tags, and fluorescent labels are signal generating reporter groups which can be detected without further modifications. Detectable moieties also include luminescent and phosphorescent groups.

The term “secondary label” as used herein refers to moieties such as biotin and various protein antigens that require the presence of a second intermediate for production of a detectable signal. For biotin, the secondary intermediate may include streptavidin-enzyme conjugates. For antigen labels, secondary intermediates may include antibody-enzyme conjugates. Some fluorescent groups act as secondary labels because they transfer energy to another group in the process of nonradiative fluorescent resonance energy transfer (FRET), and the second group produces the detected signal.

The terms “fluorescent label”, “fluorescent dye”, and “fluorophore” as used herein refer to moieties that absorb light energy at a defined excitation wavelength and emit light energy at a different wavelength. Examples of fluorescent labels include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Coumarin 343, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, Texas Red-X.

The term “mass-tag” as used herein refers to any moiety that is capable of being uniquely detected by virtue of its mass using mass spectrometry (MS) detection techniques. Examples of mass-tags include electrophore release tags such as N-[3-[4′-[(p-Methoxytetrafluorobenzyl)oxy]phenyl]-3-methylglyceronyl]isonipecotic Acid, 4′-[2,3,5,6-Tetrafluoro-4-(pentafluorophenoxyl)]methyl acetophenone, and their derivatives. The synthesis and utility of these mass-tags is described in U.S. Pat. Nos. 4,650,750, 4,709,016, 5,360,8191, 5,516,931, 5,602,273, 5,604,104, 5,610,020, and 5,650,270. Other examples of mass-tags include, but are not limited to, nucleotides, dideoxynucleotides, oligonucleotides of varying length and base composition, oligopeptides, oligosaccharides, and other synthetic polymers of varying length and monomer composition. A large variety of organic molecules, both neutral and charged (biomolecules or synthetic compounds) of an appropriate mass range (100-2000 Daltons) may also be used as mass-tags.

The terms “measurable affinity” and “measurably inhibit,” as used herein, means a measurable change in a Cbl-b ubquitination activity between a sample comprising a compound of the present invention, or composition thereof, and a Cbl-b E3 ligase, and an equivalent sample comprising an Cbl-b E3 ligase, in the absence of said compound, or composition thereof.

### 3. Description of Exemplary Embodiments

As described above, in certain embodiments, the present invention provides a compound of formula I:

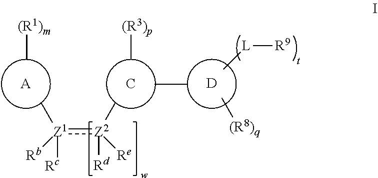

or a pharmaceutically acceptable salt thereof, wherein:
Ring A is a 5-6-membered heteroaryl ring having 1-4 heteroatoms independently selected from nitrogen, oxygen, and sulfur; or a 5-6-membered saturated or partially unsaturated heterocyclic ring having 1-4 heteroatoms independently selected from nitrogen, oxygen, and sulfur;
each R¹ is independently hydrogen, oxo, halogen, —CN, —NO₂, —CHF₂, —CF₃, —OR, —SR, —N(R)₂, —S(O)₂R, —S(O)₂N(R)₂, —S(O)R, —S(O)N(R)₂, —C(O)R, —C(O)OR, —C(O)N(R)₂, —C(O)N(R)OR, —OC(O)R, —OC(O)N(R)₂, —N(R)C(O)OR, —N(R)C(O)R, —N(R)C(O)N(R)₂, —N(R)C(NR)N(R)₂, —N(R)N(R)₂, —N(R)S(O)₂N(R)₂, —N(R)S(O)₂R, —N═S(O)(R)₂, —S(NR)(O)R, —N(R)S(O)R, —N(R)CN, —P(O)(R)N(R)₂, —P(O)(R)OR, or —P(O)(R)₂; or an optionally substituted C₁₋₆ aliphatic;
Z¹ is C or N;
Z² is C or N;
each of R^{b}, R^{c}, R^{d}, and R^{e} is independently hydrogen, halogen, —CN, —NO₂, —CHF₂, —CF₃, —OR, —SR, —N(R)₂, —S(O)₂R, —S(O)₂N(R)₂, —S(O)R, —S(O)N(R)₂, —C(O)R, —C(O)OR, —C(O)N(R)₂, —C(O)N(R)OR, —OC(O)R, —OC(O)N(R)₂, —N(R)C(O)OR, —N(R)C(O)R, —N(R)C(O)N(R)₂, —N(R)C(NR)N(R)₂, —N(R)N(R)₂, —N(R)S(O)₂N(R)₂, —N(R)S(O)₂R, —N═S(O)(R)₂, —S(NR)(O)R, —N(R)S(O)R, —N(R)CN, —P(O)(R)N(R)₂, —P(O)(R)OR, or —P(O)(R)₂; or each of R^{b}, R^{c}, R^{d}, and R^{e} is independently an optionally substituted group selected from C₁₋₆ aliphatic; phenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, phosphorous, silicon and sulfur; and a 5-6 membered monocyclic heteroaryl ring having 1-4 heteroatoms independently selected from nitrogen, oxygen, and sulfur; wherein R^{c} and R^{e} are absent if not allowed by valence; or
R^{b} and R^{c}, together with Z¹, are taken together to form an optionally substituted group selected from a 3-7 membered saturated or partially unsaturated monocyclic carbocyclic ring; a 5-6 membered monocyclic aryl ring; a 3-7 membered saturated or partially unsaturated monocyclic heterocyclic ring having 1-2 heteroatoms independently selected from nitrogen, oxygen, phosphorous, silicon and sulfur; and a 5-6 membered monocyclic heteroaryl ring having 1-4 heteroatoms independently selected from nitrogen, oxygen, and sulfur; or
R^{d} and R^{e}, together with Z², are taken together to form an optionally substituted group selected from a 3-7 membered saturated or partially unsaturated monocyclic carbocyclic ring; a 5-6 membered monocyclic aryl ring; a 3-7 membered saturated or partially unsaturated monocyclic heterocyclic ring having 1-2 heteroatoms independently selected from nitrogen, oxygen, phosphorous, silicon and sulfur; and a 5-6 membered monocyclic heteroaryl ring having 1-4 heteroatoms independently selected from nitrogen, oxygen, and sulfur; or
R^{b} and R^{d} together with the atoms to which each are attached, forms

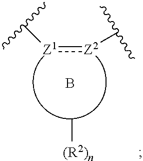

wherein Ring B is a divalent phenyl or a divalent 5-6 membered heteroaryl ring having 1-4 heteroatoms independently selected from nitrogen, oxygen, and sulfur;
each R² is independently hydrogen, oxo, halogen, —CN, —NO₂, —CHF₂, —CF₃, —OR, —CH₂OR, —OCH₂C≡CR, —CH₂N(R)₂, —SR, —N(R)₂, —S(O)₂R, —S(O)₂N(R)₂, —S(O)R, —S(O)N(R)₂, —C(O)R, —C(O)OR, —C(O)N(R)₂, —C(O)N(R)OR, —OC(O)R, —OC(O)N(R)₂, —N(R)C(O)OR, —N(R)C(O)R, —N(R)C(O)N(R)₂, —N(R)C(NR)N(R)₂, —N(R)N(R)₂, —N(R)S(O)₂N(R)₂, —N(R)S(O)₂R, —N═S(O)(R)₂, —S(NR)(O)R, —N(R)S(O)R, —N(R)CN, —P(O)(R)N(R)₂, —P(O)(R)OR, or —P(O)(R)₂; or an optionally substituted C₁₋₆ aliphatic;
Ring C is a divalent phenyl or a divalent 5-6 membered heteroaryl ring having 1-4 heteroatoms independently selected from nitrogen, oxygen, and sulfur;
each R³ is independently hydrogen, oxo, halogen, —CN, —NO₂, —CHF₂, —CF₃, —OR, —SR, —N(R)₂, —S(O)₂R, —S(O)₂N(R)₂, —S(O)R, —S(O)N(R)₂, —C(O)R, —C(O)OR, —C(O)N(R)₂, —C(O)N(R)OR, —OC(O)R, —OC(O)N(R)₂, —N(R)C(O)OR, —N(R)C(O)R, —N(R)C(O)N(R)₂, —N(R)C(NR)N(R)₂, —N(R)N(R)₂, —N(R)S(O)₂N(R)₂, —N(R)S(O)₂R, —N═S(O)(R)₂, —S(NR)(O)R, —N(R)S(O)R, —N(R)CN, —P(O)(R)N(R)₂, —P(O)(R)OR, or —P(O)(R)₂; or an optionally substituted group selected from C₁₋₆ aliphatic; a phenyl ring; a 3-7 membered saturated or partially unsaturated monocyclic carbocyclic ring; a 5-6 membered monocyclic heteroaryl ring having 1-4 heteroatoms independently selected from nitrogen, oxygen, and sulfur; and a 4-8 membered saturated or partially unsaturated monocyclic heterocyclic ring having 1-2 heteroatoms independently selected from nitrogen, oxygen, and sulfur;
   or two R³ groups, and the atoms to which each R³ group is attached, are optionally taken together to form a fused 3-7 membered saturated or partially unsaturated monocyclic carbocyclic ring; a fused 5-6 membered monocyclic aryl ring; a fused 3-7 membered saturated or partially unsaturated monocyclic heterocyclic ring having 1-2 heteroatoms independently selected from nitrogen, oxygen, phosphorous, silicon and sulfur; or a fused 5-6 membered monocyclic heteroaryl ring having 1-4 heteroatoms independently selected from nitrogen, oxygen, and sulfur;
Ring D is a 3-7 membered saturated or partially unsaturated monocyclic carbocyclic ring; a 4-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; a 5-10 membered saturated or partially unsaturated bridged bicyclic ring having 0-5 heteroatoms selected from nitrogen, oxygen, and sulfur; a 6-10 membered saturated or partially unsaturated spiro bicyclic ring having 0-5 heteroatoms independently selected from nitrogen, oxygen, and sulfur; or a 6-10 membered saturated, partially unsaturated, or unsaturated fused bicyclic ring having 0-5 heteroatoms independently selected from nitrogen, oxygen, and sulfur;
each R⁸ is independently hydrogen, oxo, halogen, —CN, —NO₂, —CHF₂, —CF₃, —OR, —SR, —N(R)₂, —S(O)₂R, —S(O)₂N(R)₂, —S(O)R, —S(O)N(R)₂, —C(O)R, —C(O)OR, —C(O)N(R)₂, —C(O)N(R)OR, —OC(O)R, —OC(O)N(R)₂, —N(R)C(O)OR, —N(R)C(O)R, —N(R)C(O)N(R)₂, —N(R)C(NR)N(R)₂, —N(R)N(R)₂, —N(R)S(O)₂N(R)₂, —N(R)S(O)₂R, —N═S(O)(R)₂, —S(NR)(O)R, —N(R)S(O)R, —N(R)CN, —P(O)(R)N(R)₂, —P(O)(R)OR, or —P(O)(R)₂; or an optionally substituted groups selected from C₁₋₆ aliphatic; phenyl; a 3-7 membered saturated or partially unsaturated monocyclic carbocyclic ring; a 4-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur;
L is a covalent bond; or L is a C₁₋₄ bivalent saturated or unsaturated, straight or branched hydrocarbon chain wherein one or two methylene units of the chain are optionally and independently replaced by —C(R)₂—, —N(R)—, —O—, —S—, —S(O)—, —S(O)₂—, —S(O)N(R)—, —N(R)S(O)—, —S(O)₂N(R)—, —N(R)S(O)₂—, —C(O)—, —C(O)O—, —OC(O)—, —C(O)N(R)—, —N(R)C(O)—, —C(O)N(R)O—, —ON(R)C(O)—, —OC(O)N(R)—, —N(R)C(O)O—, or —N(R)C(O)N(R)—;
R⁹ is halogen, —CN, —NO₂, —OR, —SR, —N(R)₂, —S(O)₂R, —S(O)₂N(R)₂, —S(O)R, —S(O)N(R)₂, —CF₂R, —CF₃, —C(R)₂OR, —C(R)₂N(R)₂, —C(O)R, —C(O)OR, —C(O)N(R)₂, —C(O)N(R)OR, —OC(O)R, —OC(O)N(R)₂, —C(S)N(R)₂, —N(R)C(O)OR, —N(R)C(O)R, —N(R)C(O)N(R)₂, —N(R)C(NR)N(R)₂, —N(R)N(R)₂, —N(R)S(O)₂N(R)₂, —N(R)S(O)₂R, —N═S(O)(R)₂, —S(NR)(O)R, —N(R)S(O)R, —N(R)CN, —Si(OR)(R)₂, —Si(R)₃, —P(O)(R)N(R)₂, —P(O)(R)OR, or —P(O)(R)₂; or R⁹ is an optionally substituted group selected from C₁₋₆ aliphatic; phenyl; a 3-7 membered saturated or partially unsaturated monocyclic carbocyclic ring; a 4-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; a 5-8 membered saturated or partially unsaturated bridged bicyclic ring having 0-3 heteroatoms selected from nitrogen, oxygen, and sulfur; a 6-10 membered saturated or partially unsaturated spiro bicyclic ring having 0-3 heteroatoms independently selected from nitrogen, oxygen, and sulfur; and an 8-10 membered partially aromatic or heteroaromatic bicyclic heterocyclic ring having 1-4 heteroatoms independently selected from nitrogen, oxygen, and sulfur;
each instance of R¹, R², R³, R⁸, and R⁹ is independently and optionally substituted with v instances of R^{A}, wherein each R^{A} is independently oxo, halogen, —CN, —NO₂, —OR, —SR, —N(R)₂, —S(O)₂R, —S(O)₂N(R)₂, —S(O)R, —S(O)N(R)₂, —C(R)₂OR, —C(O)R, —C(O)OR, —C(O)N(R)₂, —C(O)N(R)OR, —OC(O)R, —OC(O)N(R)₂, —N(R)C(O)OR, —N(R)C(O)R, —N(R)C(O)N(R)₂, —N(R)C(NR)N(R)₂, —N(R)N(R)₂, —N(R)S(O)₂N(R)₂, —N(R)S(O)₂R, —N═S(O)(R)₂, —S(NR)(O)R, —N(R)S(O)R, —N(R)CN, —P(O)(R)N(R)₂, —P(O)(R)OR, —P(O)(R)₂, or an optionally substituted group selected from C₁₋₆ aliphatic; phenyl; naphthyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, phosphorous, silicon and sulfur; a 5-6 membered monocyclic heteroaryl ring having 1-4 heteroatoms independently selected from nitrogen, oxygen, and sulfur; an 8-10 membered bicyclic heteroaryl ring having 1-5 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; and a 6-11 membered saturated or partially unsaturated bicyclic heterocyclic ring having 1-2 heteroatoms independently selected from nitrogen, oxygen, and sulfur;
each R is independently hydrogen, —CN, halogen, or an optionally substituted group selected from C₁₋₆ aliphatic; phenyl; naphthyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; a 6-11 membered saturated or partially unsaturated bicyclic carbocyclic ring having 1-2 heteroatoms independently selected from nitrogen, oxygen, and sulfur; or:
   two R groups on the same atom are optionally taken together with the atom to form an optionally substituted 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur;
m is 0, 1, 2, 3, 4, or 5;
n is 0, 1, 2, 3, or 4;
p is 0, 1, 2, 3, or 4;
q is 0, 1, 2, or 3;
t is 0 or 1;
each instance of v is independently 0, 1, 2, 3, 4, or 5;
w is 0 or 1; and
wherein [symbol] denotes a single or double bond.

As defined generally above, Ring A is a 5-6-membered heteroaryl ring having 1-4 heteroatoms independently selected from nitrogen, oxygen, and sulfur; or a 5-6-membered saturated or partially unsaturated heterocyclic ring having 1-4 heteroatoms independently selected from nitrogen, oxygen, and sulfur.

In certain embodiments, Ring A is a 5-6-membered heteroaryl ring having 1-4 heteroatoms independently selected from nitrogen, oxygen, and sulfur.

In certain embodiments. Ring A is 5-6-membered saturated or partially unsaturated heterocyclic ring having 1-4 heteroatoms independently selected from nitrogen, oxygen, and sulfur.

In certain embodiments, Ring A is a 5 membered heteroaryl ring having 1-4 heteroatoms independently selected from nitrogen, oxygen, and sulfur.

In some embodiments, Ring A is a furanyl, imidazolyl, isothiazolyl, isoxazolyl, oxadiazolyl, oxazolyl, pyrazolyl, pyrrolyl, thiazolyl, thienyl, triazinyl, or triazolyl.

In certain embodiments, Ring A is selected from

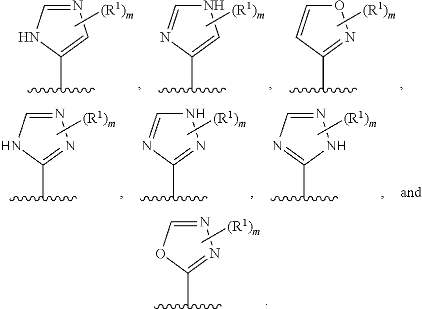

In certain embodiments, Ring A together with its R¹ substituents is

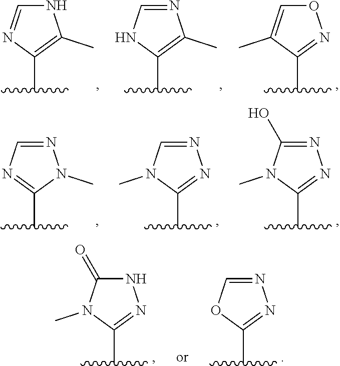

In certain embodiments, Ring A together with its R¹ substituents is

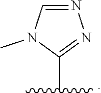 [SMILES: *c1nncn1C]

In some embodiments, Ring A is selected from those depicted in Table 1, below.

In some embodiments, Ring A together with its R¹ substituents is selected from those depicted in Table 1, below.

As defined generally above, each R¹ is independently hydrogen, oxo, halogen, —CN, —NO₂, —CHF₂, —CF₃, —OR, —SR, —N(R)₂, —S(O)₂R, —S(O)₂N(R)₂, —S(O)R, —S(O)N(R)₂, —C(O)R, —C(O)OR, —C(O)N(R)₂, —C(O)N(R)OR, —OC(O)R, —OC(O)N(R)₂, —N(R)C(O)OR, —N(R)C(O)R, —N(R)C(O)N(R)₂, —N(R)C(NR)N(R)₂, —N(R)N(R)₂, —N(R)S(O)₂N(R)₂, —N(R)S(O)₂R, —N═S(O)(R)₂, —S(NR)(O)R, —N(R)S(O)R, —N(R)CN, —P(O)(R)N(R)₂, —P(O)(R)OR, or —P(O)(R)₂; or an optionally substituted C₁₋₆ aliphatic.

In certain embodiments, R¹ is halogen, —CN, —NO₂, —CHF₂, —CF₃, —OR, —SR, —N(R)₂, —S(O)₂R, —S(O)₂N(R)₂, —S(O)R, —S(O)N(R)₂, —C(O)R, —C(O)OR, —C(O)N(R)₂, —C(O)N(R)OR, —OC(O)R, —OC(O)N(R)₂, —N(R)C(O)OR, —N(R)C(O)R, —N(R)C(O)N(R)₂, —N(R)C(NR)N(R)₂, —N(R)N(R)₂, —N(R)S(O)₂N(R)₂, —N(R)S(O)₂R, —N═S(O)(R)₂, —S(NR)(O)R, —N(R)S(O)R, —N(R)CN, —P(O)(R)N(R)₂, —P(O)(R)OR, or —P(O)(R)₂; or an optionally substituted C₁₋₆ aliphatic.

In certain embodiments, R¹ is halogen, —CN, —NO₂, —CHF₂, —CF₃, —OR, —N(R)₂, —C(O)R, —C(O)OR, —C(O)N(R)₂, —OC(O)R, —OC(O)N(R)₂, —N(R)C(O)OR, —N(R)C(O)R, —N(R)C(O)N(R)₂, —N(R)S(O)₂R, or —N(R)S(O)R; or an optionally substituted -Me, -Et, —Pr, -i-Pr, -n-Bu, -s-Bu, -t-Bu, straight chain or branched pentyl, or straight chain or branched hexyl.

In certain embodiments, R¹ is hydrogen, -Me, or —OH.

In certain embodiments, R¹ is -Me.

As defined generally above, m is 0, 1, 2, 3, 4, or 5.

In certain embodiments, m is 0, 1, 2, 3, 4, or 5.

In some embodiments, m is 0. In some embodiments, m is 1. In some embodiments, m is 2.

In some embodiments, m is 3. In some embodiments, m is 4. In some embodiments, m is 5.

In some embodiments, m is 0 or 1. In some embodiments, m is 0, 1, or 2. In some embodiments, m is 0, 1, 2, or 3. In some embodiments, m is 1 or 2. In some embodiments, m is 1, 2, or 3. In some embodiments, m is 1, 2, 3, or 4. In some embodiments, m is 2 or 3. In some embodiments, m is 2, 3, or 4. In some embodiments, m is 3 or 4. In some embodiments, m is 3, 4, or 5.

In some embodiments, m is 1 and R¹ is -Me or —OH.

In some embodiments, m is 1 and R¹ is -Me.

In some embodiments, each R¹ and m is selected from those depicted in Table 1, below.

In certain embodiments, Z¹ is C. In certain embodiments, Z¹ is N.

In certain embodiments, Z² is C. In certain embodiments, Z² is N.

In some embodiments, each of R^{b}, R^{c}, R^{d}, and R^{e} is independently hydrogen, halogen, —CN, —NO₂, —CHF₂, —CF₃, —OR, —SR, —N(R)₂, —S(O)₂R, —S(O)₂N(R)₂, —S(O)R, —S(O)N(R)₂, —C(O)R, —C(O)OR, —C(O)N(R)₂, —C(O)N(R)OR, —OC(O)R, —OC(O)N(R)₂, —N(R)C(O)OR, —N(R)C(O)R, —N(R)C(O)N(R)₂, —N(R)C(NR)N(R)₂, —N(R)N(R)₂, —N(R)S(O)₂N(R)₂, —N(R)S(O)₂R, —N═S(O)(R)₂, —S(NR)(O)R, —N(R)S(O)R, —N(R)CN, —P(O)(R)N(R)₂, —P(O)(R)OR, or —P(O)(R)₂; or each of R^{b}, R^{c}, R^{d}, and R^{e} is independently an optionally substituted group selected from C₁₋₆ aliphatic; phenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, phosphorous, silicon and sulfur; and a 5-6 membered monocyclic heteroaryl ring having 1-4 heteroatoms independently selected from nitrogen, oxygen, and sulfur; wherein R^{c} and R^{e} are absent if not allowed by valence.

In some embodiments, R^{b} and R^{c}, together with Z¹, are taken together to form an optionally substituted group selected from a 3-7 membered saturated or partially unsaturated monocyclic carbocyclic ring; a 5-6 membered monocyclic aryl ring; a 3-7 membered saturated or partially unsaturated monocyclic heterocyclic ring having 1-2 heteroatoms independently selected from nitrogen, oxygen, phosphorous, silicon and sulfur; and a 5-6 membered monocyclic heteroaryl ring having 1-4 heteroatoms independently selected from nitrogen, oxygen, and sulfur.

In some embodiments, R^{d}, and R^{e}, together with Z², are taken together to form an optionally substituted group selected from a 3-7 membered saturated or partially unsaturated monocyclic carbocyclic ring; a 5-6 membered monocyclic aryl ring; a 3-7 membered saturated or partially unsaturated monocyclic heterocyclic ring having 1-2 heteroatoms independently selected from nitrogen, oxygen, phosphorous, silicon and sulfur; and a 5-6 membered monocyclic heteroaryl ring having 1-4 heteroatoms independently selected from nitrogen, oxygen, and sulfur.

In some embodiments, the group

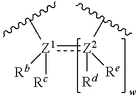

is

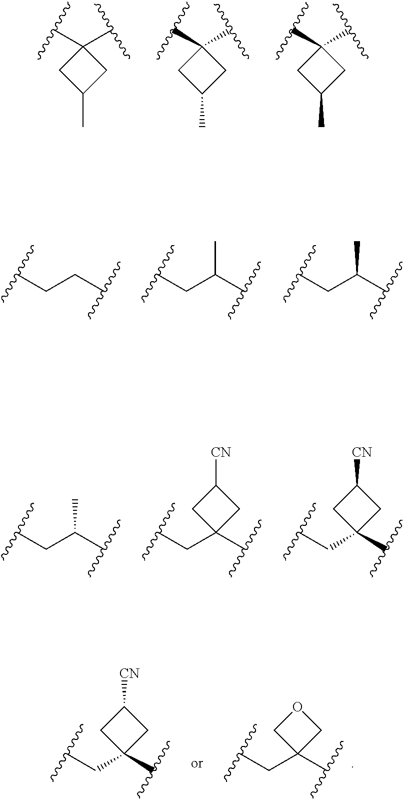

In some embodiments,

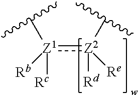

is selected from those depicted in Table 1, below.

In certain embodiments, R^{b} and R^{d} together with the atoms to which each are attached, and Z¹ and Z², forms

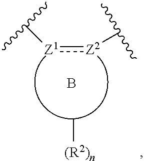

wherein Ring B is a divalent phenyl or a divalent 5-6 membered heteroaryl ring having 1-4 heteroatoms independently selected from nitrogen, oxygen, and sulfur.

In some embodiments, Ring B is a divalent phenyl, furanyl, imidazolyl, isothiazolyl, isoxazolyl, oxadiazolyl, oxazolyl, pyrimidinyl, pyrazinyl, pyrazolyl, pyridazinyl, pyridinyl, pyrimidinyl, pyrrolyl, thiazolyl, thienyl, triazinyl, or triazolyl.

In some embodiments, Ring B is a divalent phenyl, pyrazolyl, imidazolyl, or pyridinyl.

In some embodiments, Ring B is a divalent group selected from

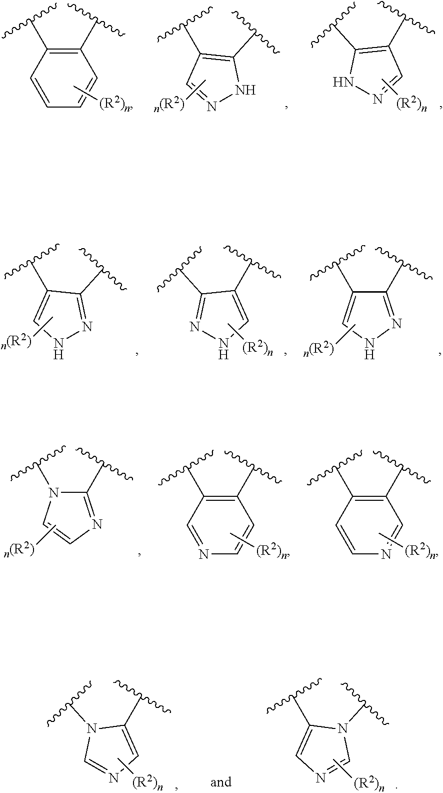

In certain embodiments, Ring B together with its R² substituents is

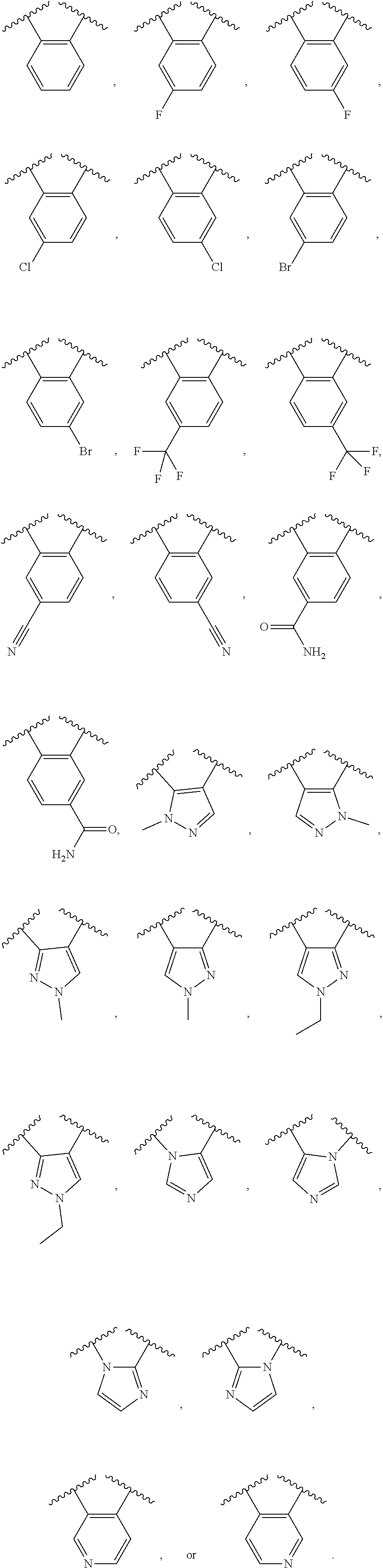

In certain embodiments, Ring B together with its R² substituents is

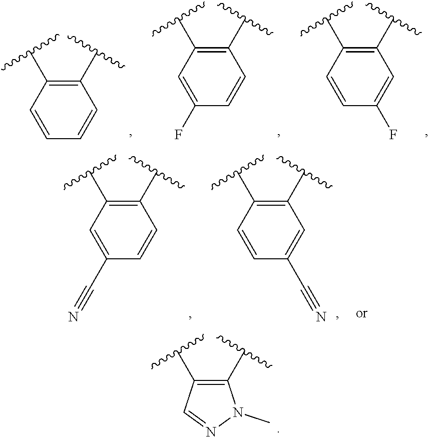

In some embodiments, Ring B is selected from those depicted in Table 1, below.

In some embodiments, Ring B together with its R² substituents is selected from those depicted in Table 1, below.

As defined generally above, each R² is independently hydrogen, oxo, halogen, —CN, —NO₂, —CHF₂, —CF₃, —OR, —CH₂OR, —OCH₂C≡CR, —CH₂N(R)₂, —SR, —N(R)₂, —S(O)₂R, —S(O)₂N(R)₂, —S(O)R, —S(O)N(R)₂, —C(O)R, —C(O)OR, —C(O)N(R)₂, —C(O)N(R)OR, —OC(O)R, —OC(O)N(R)₂, —N(R)C(O)OR, —N(R)C(O)R, —N(R)C(O)N(R)₂, —N(R)C(NR)N(R)₂, —N(R)N(R)₂, —N(R)S(O)₂N(R)₂, —N(R)S(O)₂R, —N═S(O)(R)₂, —S(NR)(O)R, —N(R)S(O)R, —N(R)CN, —P(O)(R)N(R)₂, —P(O)(R)OR, or —P(O)(R)₂; or an optionally substituted C₁₋₆ aliphatic.

In certain embodiments, R² is hydrogen, oxo, halogen, —CN, —NO₂, —CHF₂, —CF₃, —OR, —SR, —N(R)₂, —S(O)₂R, —S(O)₂N(R)₂, —S(O)R, —S(O)N(R)₂, —C(O)R, —C(O)OR, —C(O)N(R)₂, —C(O)N(R)OR, —OC(O)R, —OC(O)N(R)₂, —N(R)C(O)OR, —N(R)C(O)R, —N(R)C(O)N(R)₂, —N(R)C(NR)N(R)₂, —N(R)N(R)₂, —N(R)S(O)₂N(R)₂, —N(R)S(O)₂R, —N═S(O)(R)₂, —S(NR)(O)R, —N(R)S(O)R, —N(R)CN, —P(O)(R)N(R)₂, —P(O)(R)OR, or —P(O)(R)₂; or an optionally substituted C₁₋₆ aliphatic.

In certain embodiments, R² is halogen, —CN, —NO₂, —CHF₂, —CF₃, —OR, —SR, —N(R)₂, —S(O)₂P, —S(O)₂N(R)₂, —S(O)R, —S(O)N(R)₂, —C(O)R, —C(O)OR, —C(O)N(R)₂, —OC(O)R, —OC(O)N(R)₂, —N(R)C(O)OR, —N(R)C(O)R, —N(R)C(O)N(R)₂, —N(R)S(O)₂R, —N(R)S(O)R, or an optionally substituted C₁₋₆ aliphatic.

In certain embodiments, R² is an optionally substituted -Me, -Et, —Pr, -i-Pr, -n-Bu, -s-Bu, -t-Bu, straight chain or branched pentyl, or straight chain or branched hexyl.

In certain embodiments, R² is hydrogen, -Me, -Et, —CF₃, —F, —Cl, —Br, —CN, or —C(O)NH₂.

In certain embodiments, R² is hydrogen, -Me, —F, or —CN.

As defined generally above, n is 0, 1, 2, 3, or 4.

In certain embodiments, n is 0, 1, 2, 3, or 4.

In certain embodiments, n is 0. In certain embodiments, n is 1. In certain embodiments, n is 2. In certain embodiments, n is 3. In certain embodiments, n is 4.

In some embodiments, n is 0 or 1. In some embodiments, n is 0, 1, or 2. In some embodiments, n is 0, 1, 2, or 3. In some embodiments, n is 1 or 2. In some embodiments, n is 1, 2, or 3. In some embodiments, n is 1, 2, 3, or 4. In some embodiments, n is 2 or 3. In some embodiments, n is 2, 3, or 4. In some embodiments, n is 3 or 4.

In some embodiments, n is 1 and R² is -Me, —F, or —CN.

In some embodiments, each R² and n is selected from those depicted in Table 1, below.

As defined generally above, Ring C is a divalent phenyl or a divalent 5-6 membered heteroaryl ring having 1-4 heteroatoms independently selected from nitrogen, oxygen, and sulfur.

In some embodiments, Ring C is a divalent phenyl or a divalent 5-6 membered heteroaryl ring having 1-4 heteroatoms independently selected from nitrogen, oxygen, and sulfur.

In some embodiments, Ring C is a divalent phenyl, pyrimidinyl, pyrazinyl, pyridazinyl, pyridinyl, or pyrimidinyl.

In certain embodiments, Ring C is

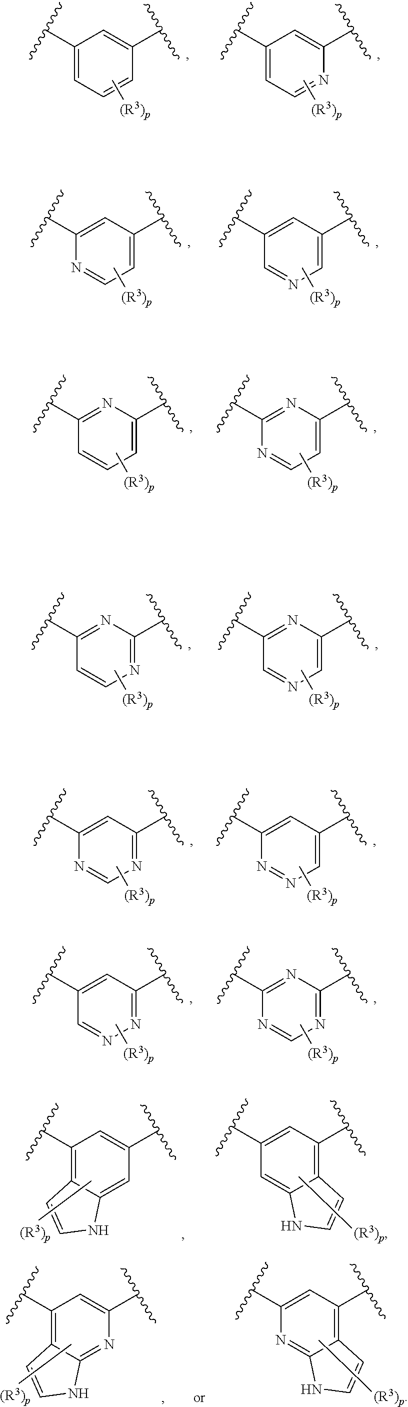

In certain embodiments, Ring C is

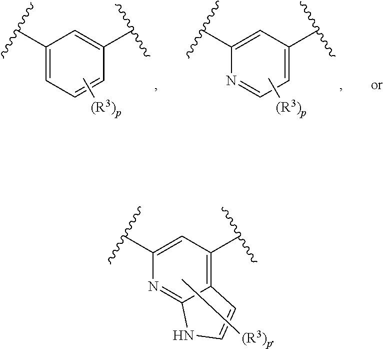

In some embodiments, Ring C is selected from those depicted in Table 1, below.

In certain embodiments, Ring C together with its R³ substituents is

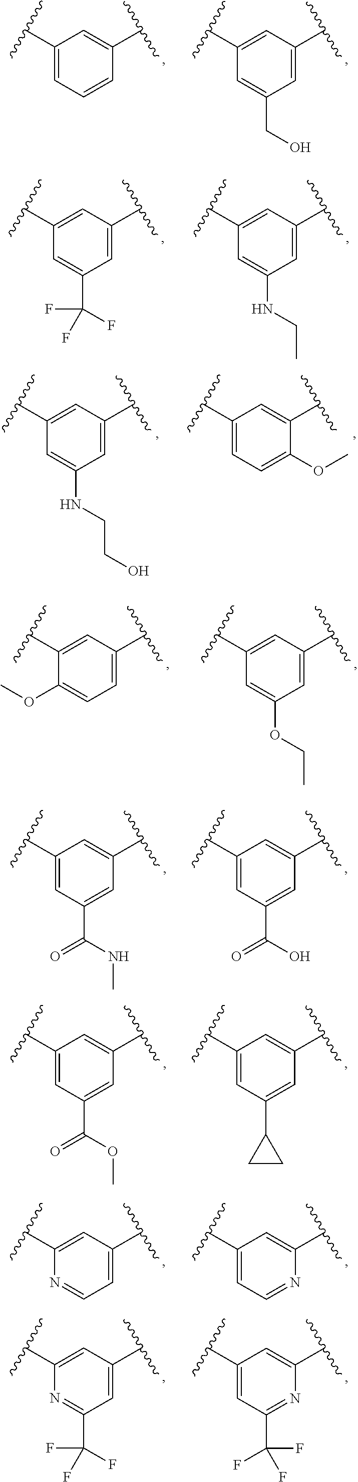

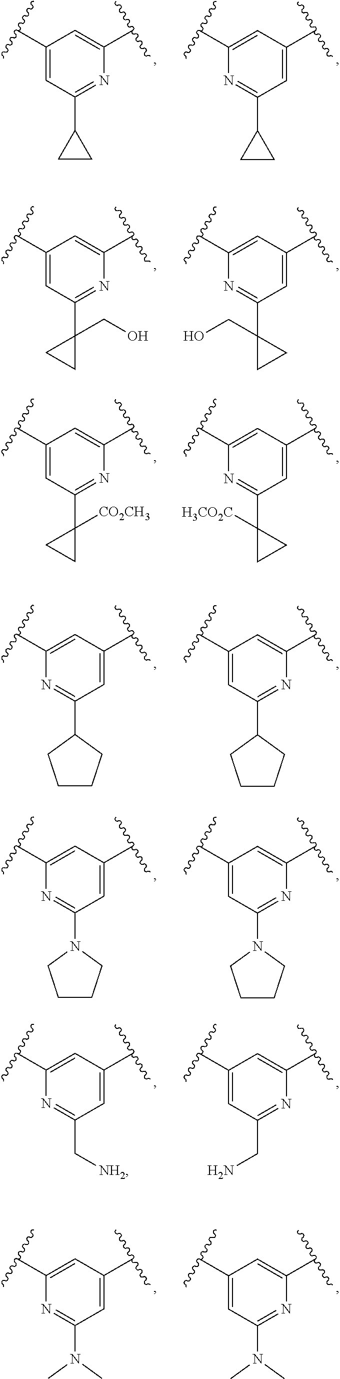

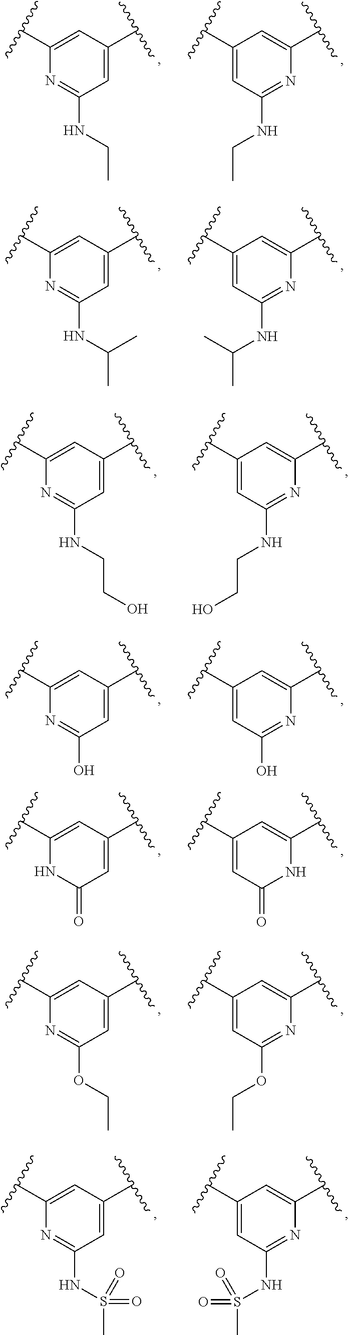

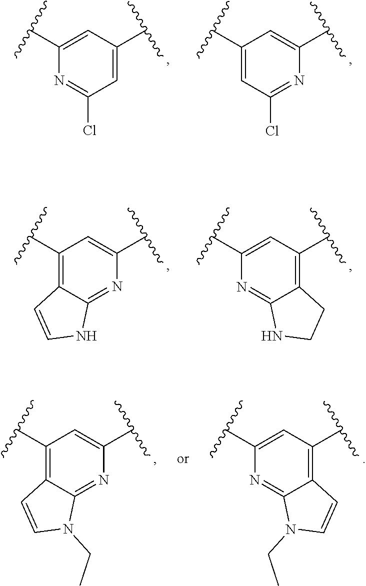 [SMILES: *c1cc(*)nc(Cl)c1 ; *c1cc(*)nc(Cl)c1 ; *c1cc(*)c2cc[nH]c2n1 ; *c1cc(*)c2c(n1)NCC2 ; *c1cc(*)c2ccn(CC)c2n1 ; *c1cc(*)c2ccn(CC)c2n1]

In certain embodiments, Ring C together with its R³ substituents is

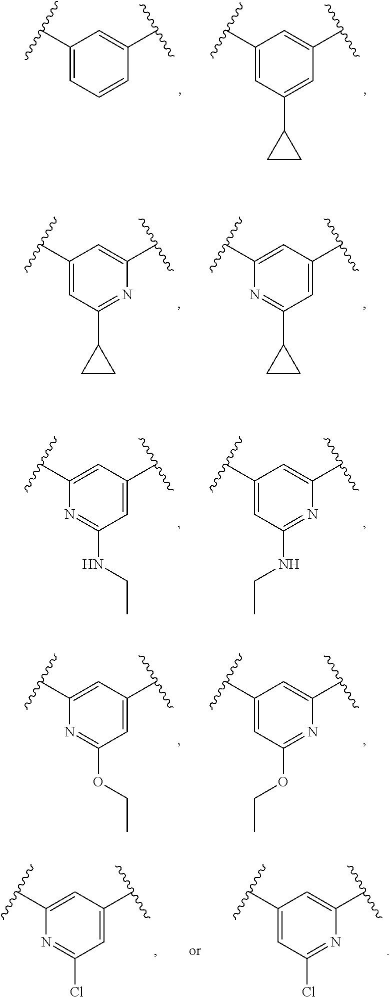

In some embodiments, Ring C together with its R³ substituents is selected from those depicted in Table 1, below.

As defined generally above, each R³ is independently hydrogen, oxo, halogen, —CN, —NO₂, —CHF₂, —CF₃, —OR, —SR, —N(R)₂, —S(O)₂R, —S(O)₂N(R)₂, —S(O)R, —S(O)N(R)₂, —C(O)R, —C(O)OR, —C(O)N(R)₂, —C(O)N(R)OR, —OC(O)R, —OC(O)N(R)₂, —N(R)C(O)OR, —N(R)C(O)R, —N(R)C(O)N(R)₂, —N(R)C(NR)N(R)₂, —N(R)N(R)₂, —N(R)S(O)₂N(R)₂, —N(R)S(O)₂R, —N═S(O)(R)₂, —S(NR)(O)R, —N(R)S(O)R, —N(R)CN, —P(O)(R)N(R)₂, —P(O)(R)OR, or —P(O)(R)₂; or an optionally substituted group selected from C₁₋₆ aliphatic; a phenyl ring; a 3-7 membered saturated or partially unsaturated monocyclic carbocyclic ring; a 5-6 membered monocyclic heteroaryl ring having 1-4 heteroatoms independently selected from nitrogen, oxygen, and sulfur; and a 4-8 membered saturated or partially unsaturated monocyclic heterocyclic ring having 1-2 heteroatoms independently selected from nitrogen, oxygen, and sulfur, or two R³ groups, and the atoms to which each R³ group is attached, are optionally taken together to form a fused 3-7 membered saturated or partially unsaturated monocyclic carbocyclic ring; a fused 5-6 membered monocyclic aryl ring; a fused 3-7 membered saturated or partially unsaturated monocyclic heterocyclic ring having 1-2 heteroatoms independently selected from nitrogen, oxygen, phosphorous, silicon and sulfur; or a fused 5-6 membered monocyclic heteroaryl ring having 1-4 heteroatoms independently selected from nitrogen, oxygen, and sulfur.

In certain embodiments, R³ is hydrogen, oxo, halogen, —CN, —NO₂, —CHF₂, —CF₃, —OR, —SR, —N(R)₂, —S(O)₂R, —S(O)₂N(R)₂, —S(O)R, —S(O)N(R)₂, —C(O)R, —C(O)OR, —C(O)N(R)₂, —C(O)N(R)OR, —OC(O)R, —OC(O)N(R)₂, —N(R)C(O)OR, —N(R)C(O)R, —N(R)C(O)N(R)₂, —N(R)C(NR)N(R)₂, —N(R)N(R)₂, —N(R)S(O)₂N(R)₂, —N(R)S(O)₂R, —N═S(O)(R)₂, —S(NR)(O)R, —N(R)S(O)R, —N(R)CN, —P(O)(R)N(R)₂, —P(O)(R)OR, or —P(O)(R)₂; or an optionally substituted group selected from C₁₋₆ aliphatic; a phenyl ring; a 3-7 membered saturated or partially unsaturated monocyclic carbocyclic ring; a 5-6 membered monocyclic heteroaryl ring having 1-4 heteroatoms independently selected from nitrogen, oxygen, and sulfur; and a 4-8 membered saturated or partially unsaturated monocyclic heterocyclic ring having 1-2 heteroatoms independently selected from nitrogen, oxygen, and sulfur.

In certain embodiments, two R³ groups, and the atoms to which each R³ group is attached, are taken together to form a fused 3-7 membered saturated or partially unsaturated monocyclic carbocyclic ring; a fused 5-6 membered monocyclic aryl ring; a fused 3-7 membered saturated or partially unsaturated monocyclic heterocyclic ring having 1-2 heteroatoms independently selected from nitrogen, oxygen, phosphorous, silicon and sulfur; or a fused 5-6 membered monocyclic heteroaryl ring having 1-4 heteroatoms independently selected from nitrogen, oxygen, and sulfur.

In certain embodiments, R³ is hydrogen, oxo, halogen, —CN, —NO₂, —OR, —SR, —N(R)₂, —S(O)₂R, —S(O)₂N(R)₂, —S(O)R, —S(O)N(R)₂, —C(O)R, —C(O)OR, —C(O)N(R)₂, —C(O)N(R)OR, —OC(O)R, —OC(O)N(R)₂, —N(R)C(O)OR, —N(R)C(O)R, —N(R)C(O)N(R)₂, —N(R)C(NR)N(R)₂, —N(R)N(R)₂, —N(R)S(O)₂N(R)₂, —N(R)S(O)₂R, —N═S(O)(R)₂, —S(NR)(O)R, —N(R)S(O)R, —N(R)CN; or an optionally substituted C₁₋₆ aliphatic; or an optionally substituted 3-7 membered saturated or partially unsaturated monocyclic carbocyclic ring.

In certain embodiments, each R³ is independently —H, halogen, —OR, —NR₂, —C(O)R, —C(O)OR, —C(O)NR₂, —C(O)N(R)OR, —N(R)C(O)OR, —N(R)C(O)R, —N(R)C(O)NR₂, —N(R)S(O)₂R, or —N(R)S(O)R; or each instance of R³ is independently an optionally substituted C₁₋₆ aliphatic; or a 3-7 membered saturated or partially unsaturated monocyclic carbocyclic ring.

In certain embodiments, R³ is hydrogen, oxo, -Me, -Et, —Pr, -i-Pr, straight chain or branched butyl, straight chain or branched pentyl, straight chain or branched hexyl, —CF₃, —F, —Cl, —Br, —OH, —OMe, —OEt, —NH₂,

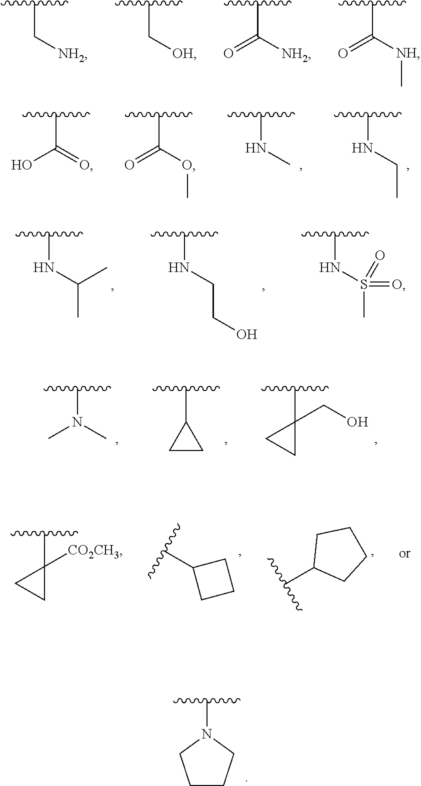

In certain embodiments, R³ is hydrogen, -Me, -Et, —CF₃, —Cl, —OH, —OMe, —OEt,

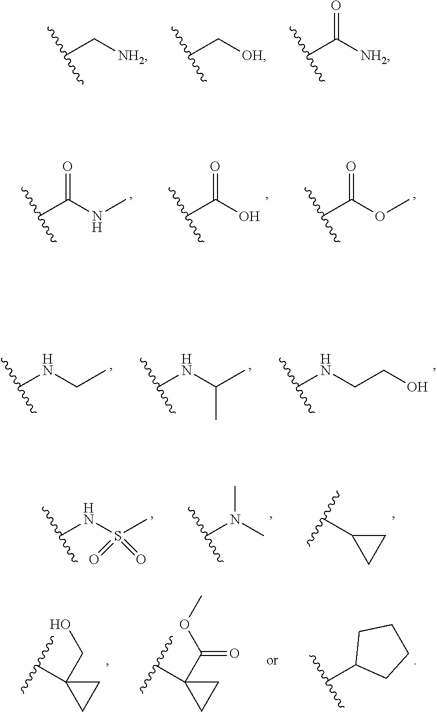

In certain embodiments, R³ is hydrogen or cyclopropyl.

As defined generally above, p is 0, 1, 2, 3, or 4.

In certain embodiments, p is 0, 1, 2, 3, or 4.

In certain embodiments, p is 0. In certain embodiments, p is 1. In certain embodiments, p is 2. In certain embodiments, p is 3. In certain embodiments, p is 4.

In some embodiments, p is 0 or 1. In some embodiments, p is 0, 1, or 2. In some embodiments, p is 0, 1, 2, or 3. In some embodiments, p is 0, 1, 2, 3, or 4. In some embodiments, p is 1 or 2. In some embodiments, p is 1, 2, or 3. In some embodiments, p is 1, 2, 3, or 4. In some embodiments, p is 2 or 3. In some embodiments, p is 2, 3, or 4. In some embodiments, p is 3 or 4.

In some embodiments, p is 1 and R³ is hydrogen, cyclopropyl, —Cl, OEt, or —NHEt.

In some embodiments, p is 1 and R³ is hydrogen or cyclopropyl.

In some embodiments, each R and p is selected from those depicted in Table 1, below.

In certain embodiments, Ring D is a 4-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; or a 6-10 membered saturated, partially unsaturated, or fully unsaturated fused bicyclic ring having 0-5 heteroatoms independently selected from nitrogen, oxygen, and sulfur.

In certain embodiments, Ring D is selected from

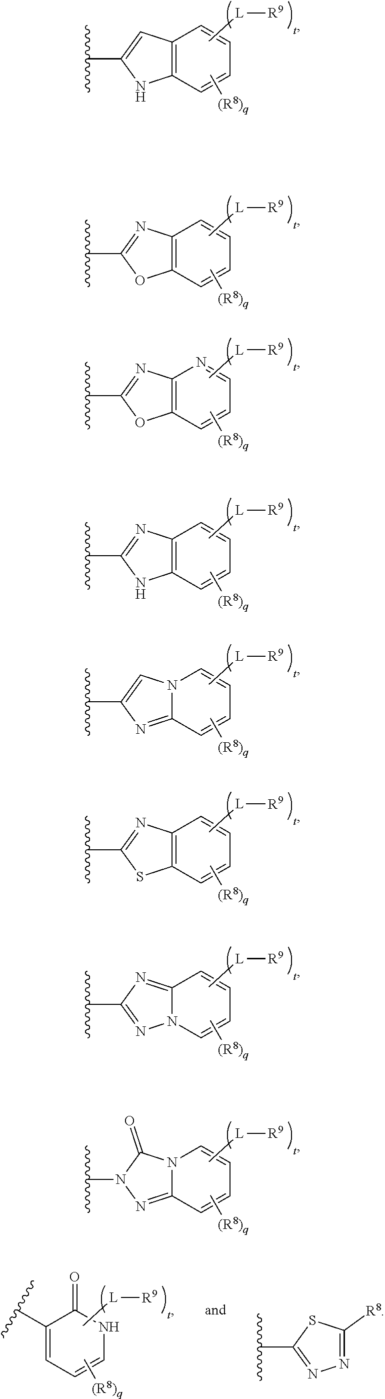

In certain embodiments, Ring D, along with R⁸ and L-R⁹, is selected from

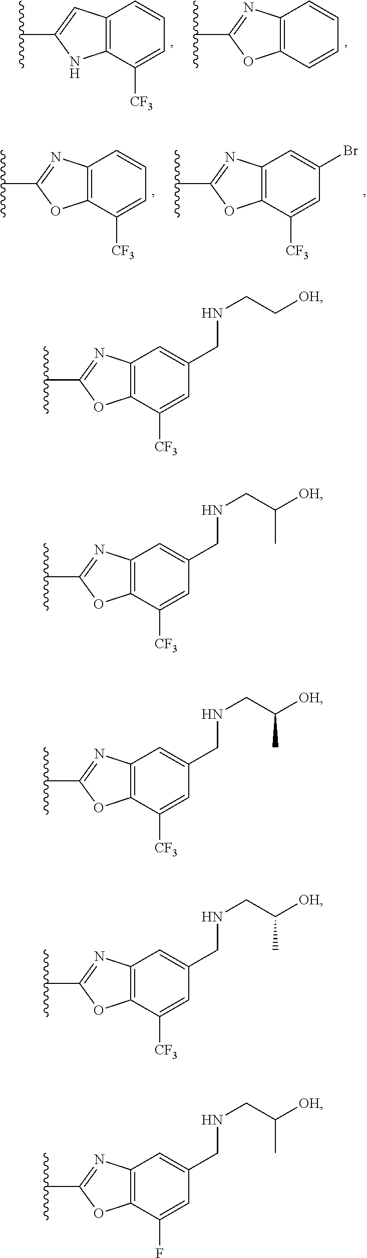

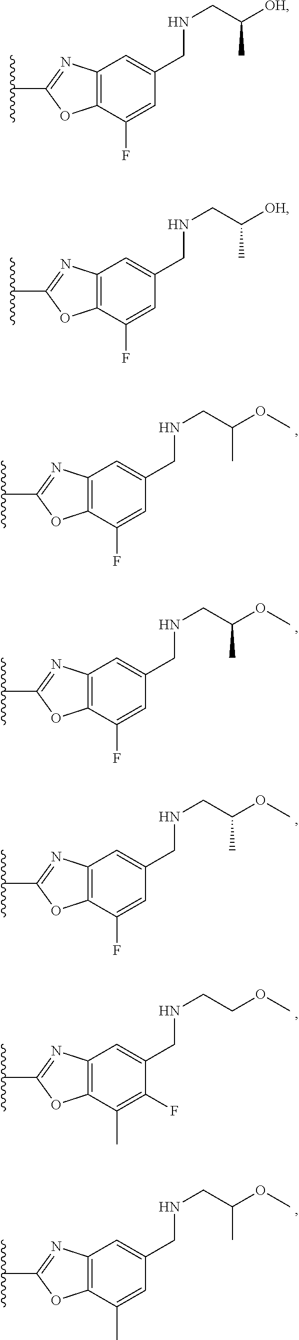

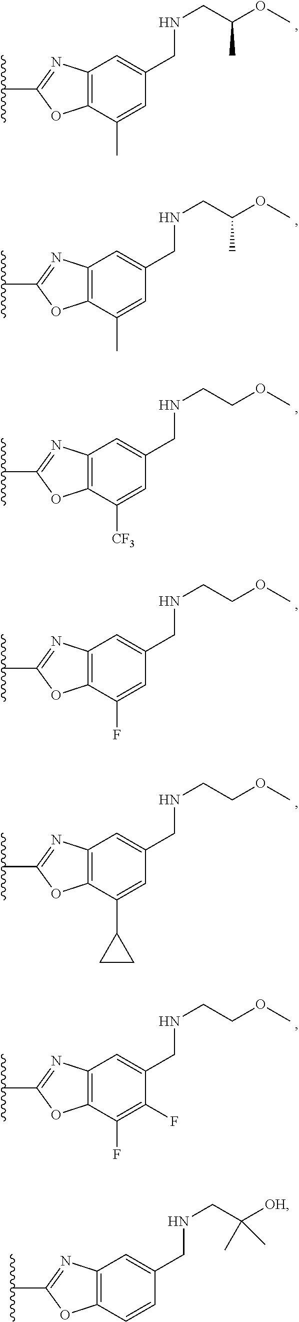

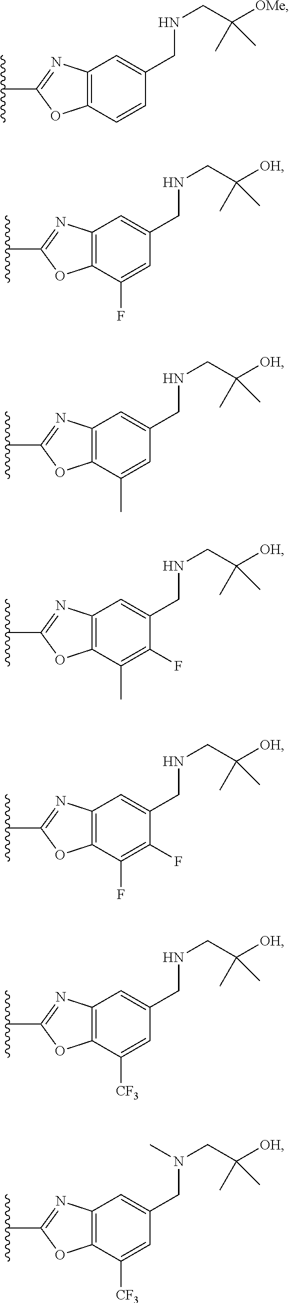

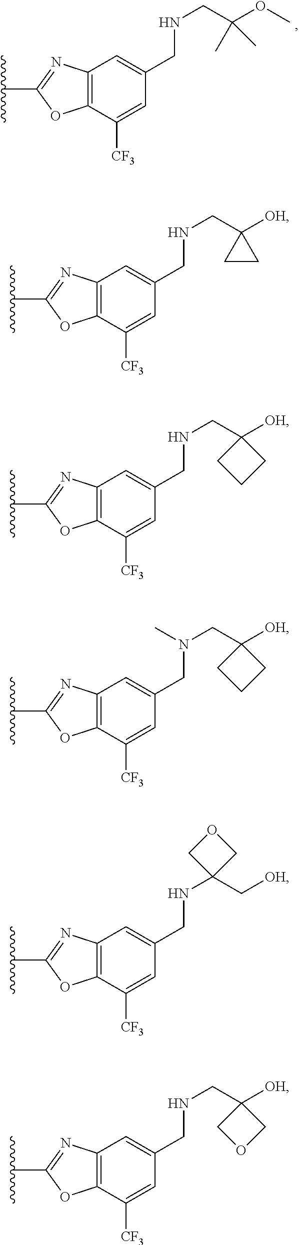 [SMILES: *c1nc2cc(CNCC(C)(C)OC)cc(C(F)(F)F)c2o1 ; *c1nc2cc(CNCC3(O)CC3)cc(C(F)(F)F)c2o1 ; *c1nc2cc(CNCC3(O)CCC3)cc(C(F)(F)F)c2o1 ; *c1nc2cc(CN(C)CC3(O)CCC3)cc(C(F)(F)F)c2o1 ; *c1nc2cc(CNC3(CO)COC3)cc(C(F)(F)F)c2o1 ; *c1nc2cc(CNCC3(O)COC3)cc(C(F)(F)F)c2o1]

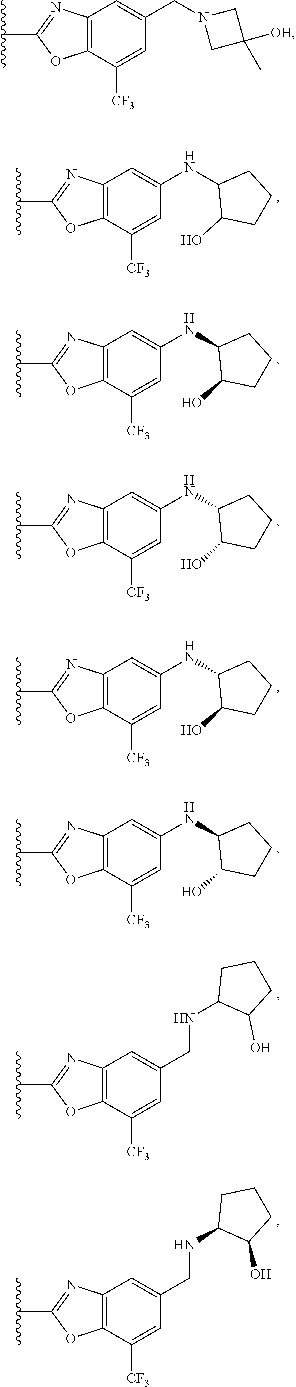

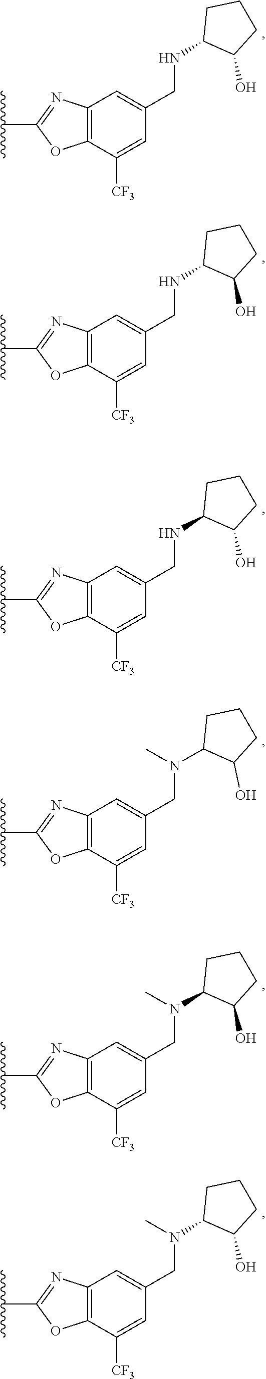 [SMILES: *c1nc2cc(CN[C@@H]3CCC[C@@H]3O)cc(C(F)(F)F)c2o1 ; *c1nc2cc(CN[C@@H]3CCC[C@H]3O)cc(C(F)(F)F)c2o1 ; *c1nc2cc(CN[C@H]3CCC[C@@H]3O)cc(C(F)(F)F)c2o1 ; *c1nc2cc(CN(C)C3CCCC3O)cc(C(F)(F)F)c2o1 ; *c1nc2cc(CN(C)[C@H]3CCC[C@H]3O)cc(C(F)(F)F)c2o1 ; *c1nc2cc(CN(C)[C@@H]3CCC[C@@H]3O)cc(C(F)(F)F)c2o1]

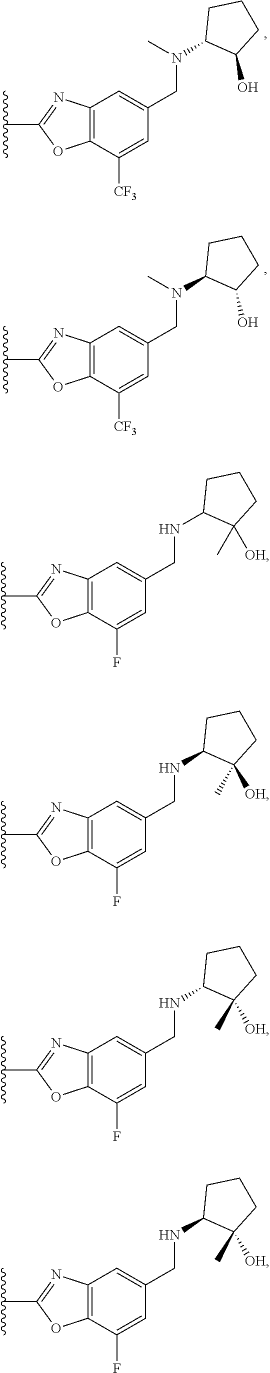 [SMILES: *c1nc2cc(CN(C)[C@@H]3CCC[C@H]3O)cc(C(F)(F)F)c2o1 ; *c1nc2cc(CN(C)[C@H]3CCC[C@@H]3O)cc(C(F)(F)F)c2o1 ; *c1nc2cc(CNC3CCCC3(C)O)cc(F)c2o1 ; *c1nc2cc(CN[C@H]3CCC[C@@]3(C)O)cc(F)c2o1 ; *c1nc2cc(CN[C@@H]3CCC[C@]3(C)O)cc(F)c2o1 ; *c1nc2cc(CN[C@H]3CCC[C@]3(C)O)cc(F)c2o1]

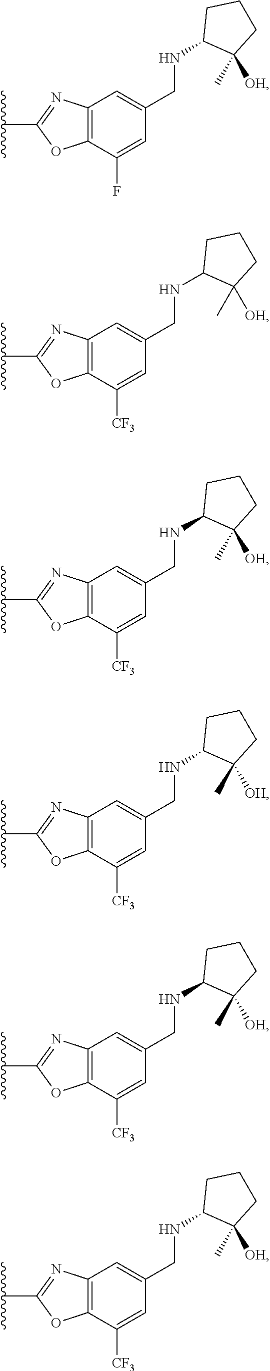 [SMILES: *c1nc2cc(CN[C@@H]3CCC[C@@]3(C)O)cc(F)c2o1 ; *c1nc2cc(CNC3CCCC3(C)O)cc(C(F)(F)F)c2o1 ; *c1nc2cc(CN[C@H]3CCC[C@@]3(C)O)cc(C(F)(F)F)c2o1 ; *c1nc2cc(CN[C@@H]3CCC[C@]3(C)O)cc(C(F)(F)F)c2o1 ; *c1nc2cc(CN[C@H]3CCC[C@]3(C)O)cc(C(F)(F)F)c2o1 ; *c1nc2cc(CN[C@@H]3CCC[C@@]3(C)O)cc(C(F)(F)F)c2o1]

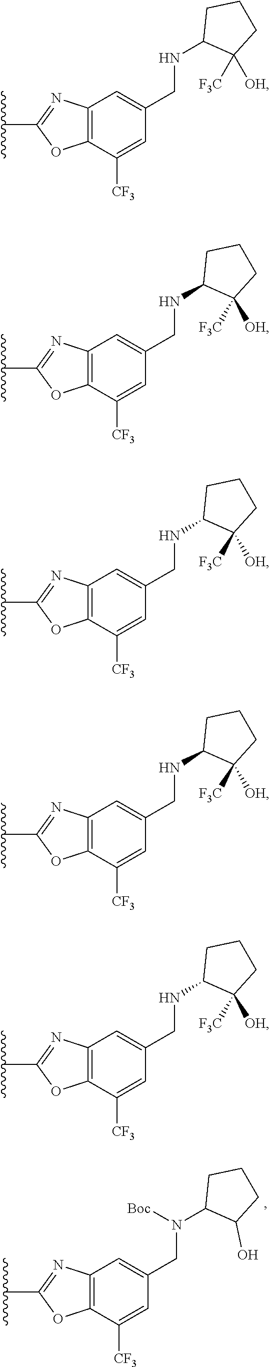 [SMILES: *c1nc2cc(CNC3CCCC3(O)C(F)(F)F)cc(C(F)(F)F)c2o1 ; *c1nc2cc(CN[C@H]3CCC[C@]3(O)C(F)(F)F)cc(C(F)(F)F)c2o1 ; *c1nc2cc(CN[C@@H]3CCC[C@@]3(O)C(F)(F)F)cc(C(F)(F)F)c2o1 ; *c1nc2cc(CN[C@H]3CCC[C@@]3(O)C(F)(F)F)cc(C(F)(F)F)c2o1 ; *c1nc2cc(CN[C@@H]3CCC[C@]3(O)C(F)(F)F)cc(C(F)(F)F)c2o1 ; *c1nc2cc(CN(C(=O)OC(C)(C)C)C3CCCC3O)cc(C(F)(F)F)c2o1]

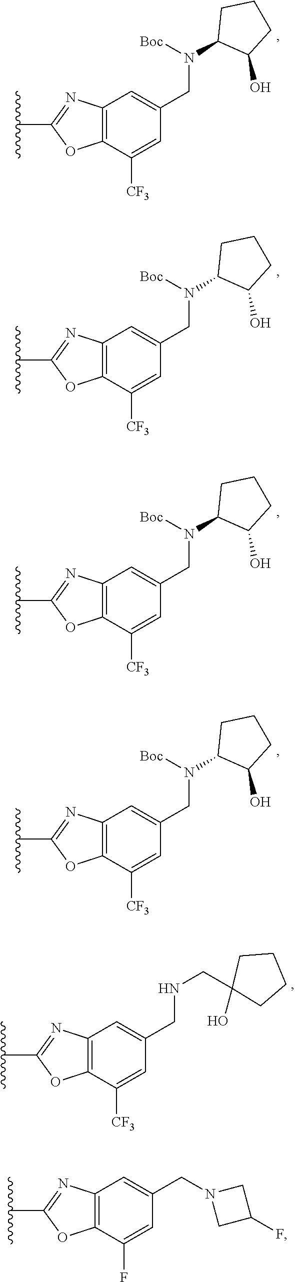 [SMILES: *c1nc2cc(CN(C(=O)OC(C)(C)C)[C@H]3CCC[C@H]3O)cc(C(F)(F)F)c2o1 ; *c1nc2cc(CN(C(=O)OC(C)(C)C)[C@@H]3CCC[C@@H]3O)cc(C(F)(F)F)c2o1 ; *c1nc2cc(CN(C(=O)OC(C)(C)C)[C@H]3CCC[C@@H]3O)cc(C(F)(F)F)c2o1 ; *c1nc2cc(CN(C(=O)OC(C)(C)C)[C@@H]3CCC[C@H]3O)cc(C(F)(F)F)c2o1 ; *c1nc2cc(CNCC3(O)CCCC3)cc(C(F)(F)F)c2o1 ; *c1nc2cc(CN3CC(F)C3)cc(F)c2o1]

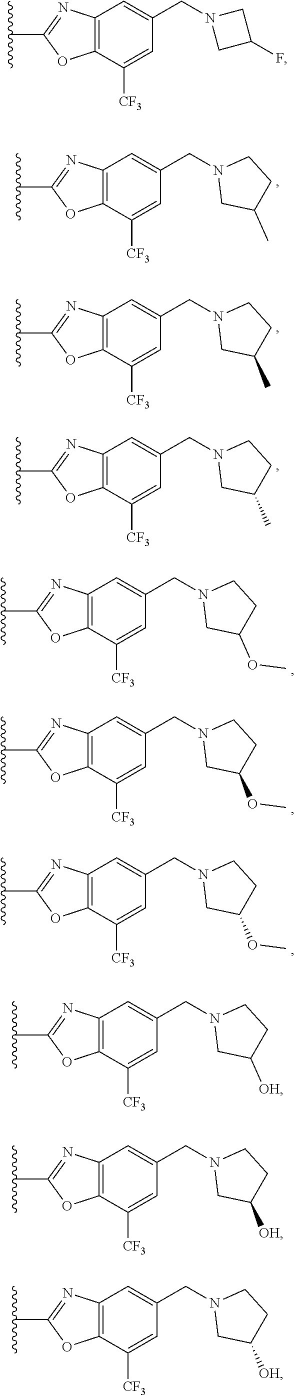

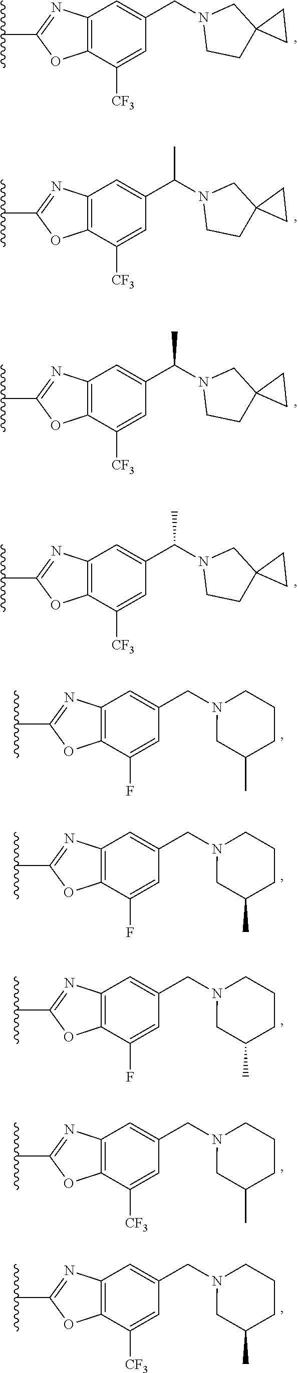

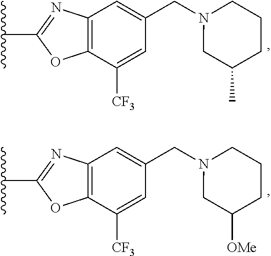

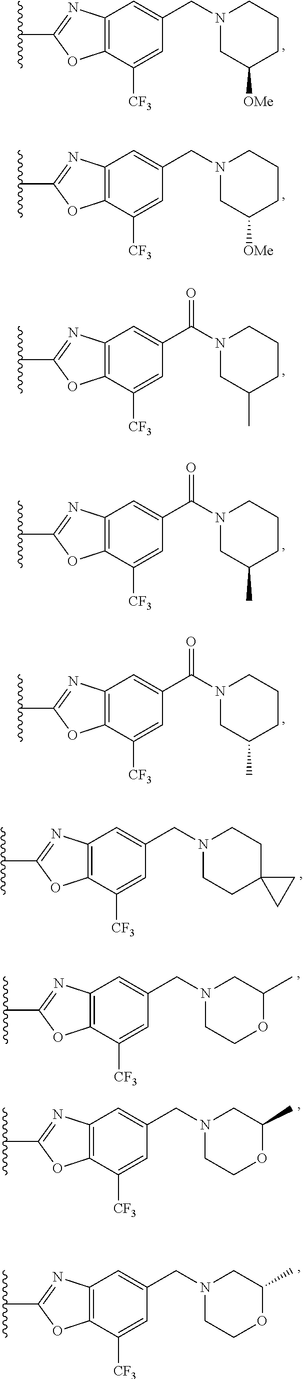

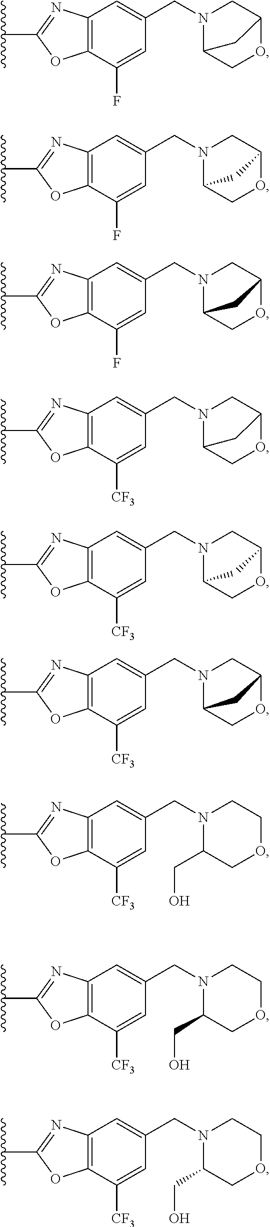

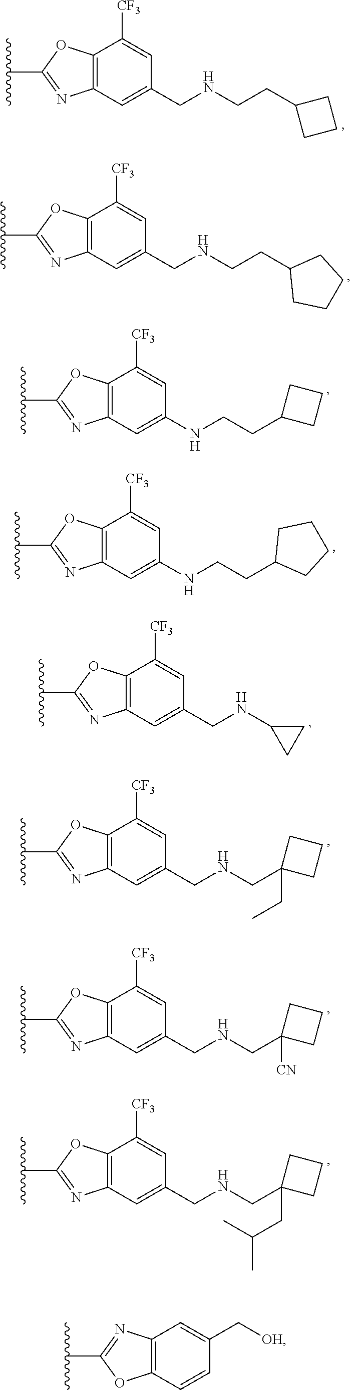

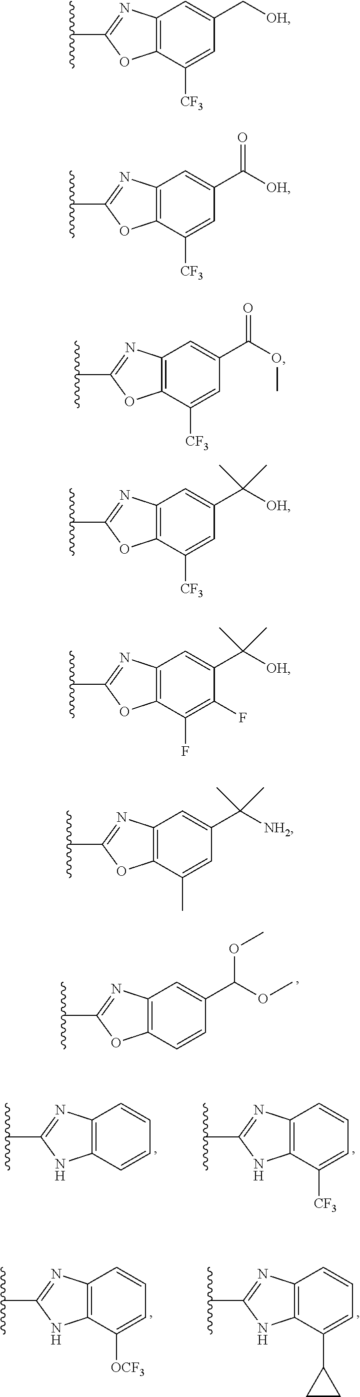

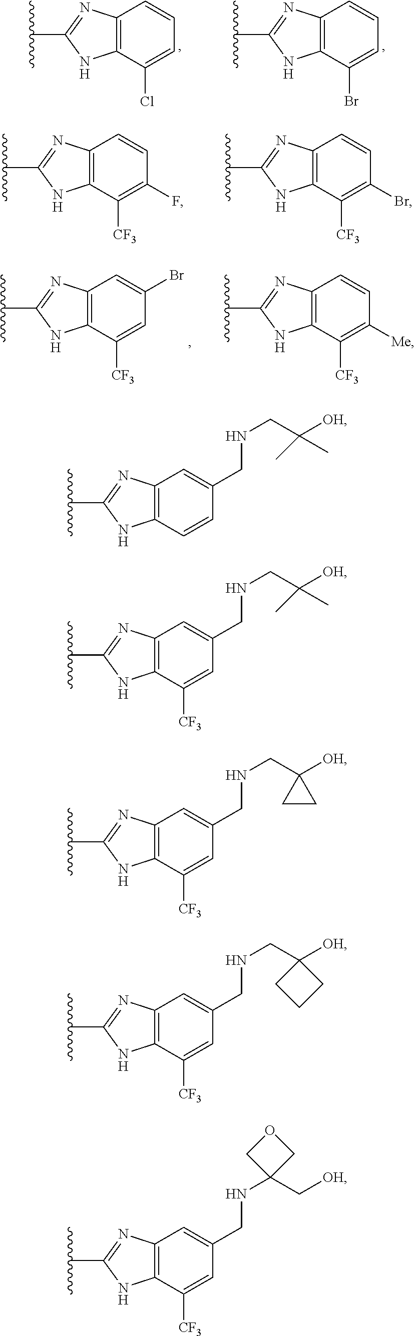

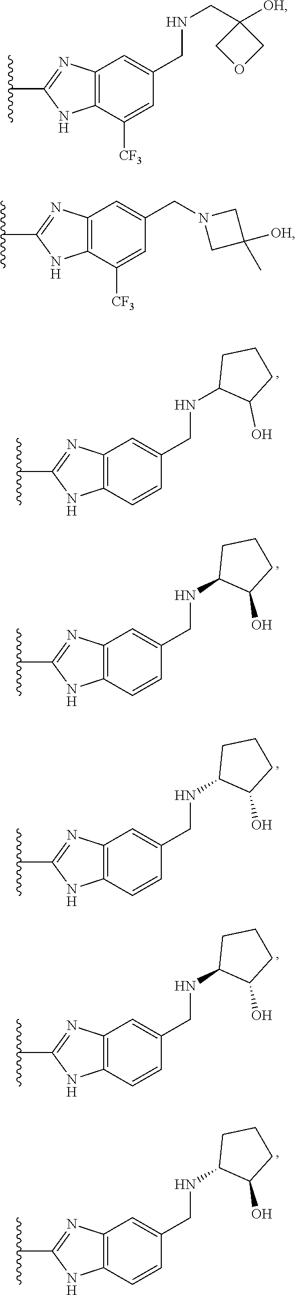

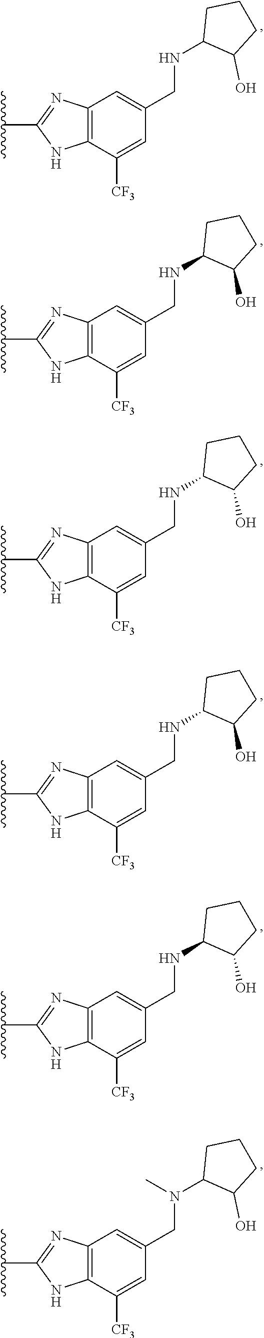 [SMILES: *c1nc2cc(CNC3CCCC3O)cc(C(F)(F)F)c2[nH]1 ; *c1nc2cc(CN[C@H]3CCC[C@H]3O)cc(C(F)(F)F)c2[nH]1 ; *c1nc2cc(CN[C@@H]3CCC[C@@H]3O)cc(C(F)(F)F)c2[nH]1 ; *c1nc2cc(CN[C@@H]3CCC[C@H]3O)cc(C(F)(F)F)c2[nH]1 ; *c1nc2cc(CN[C@H]3CCC[C@@H]3O)cc(C(F)(F)F)c2[nH]1 ; *c1nc2cc(CN(C)C3CCCC3O)cc(C(F)(F)F)c2[nH]1]

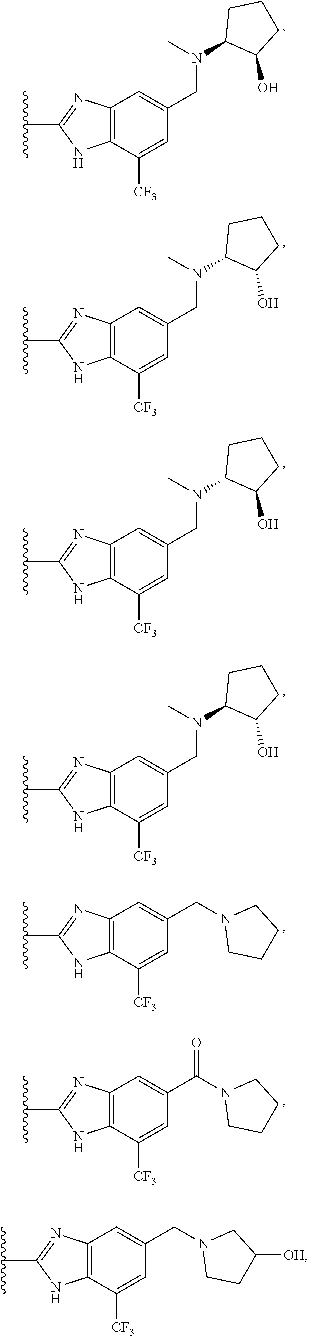

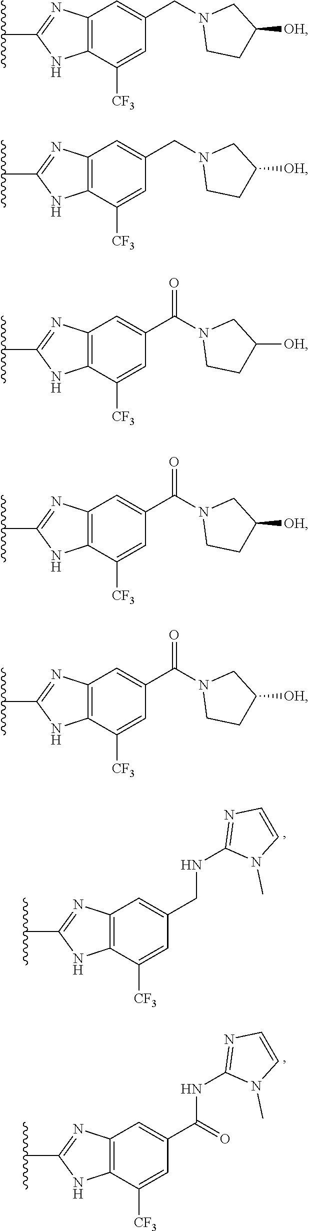

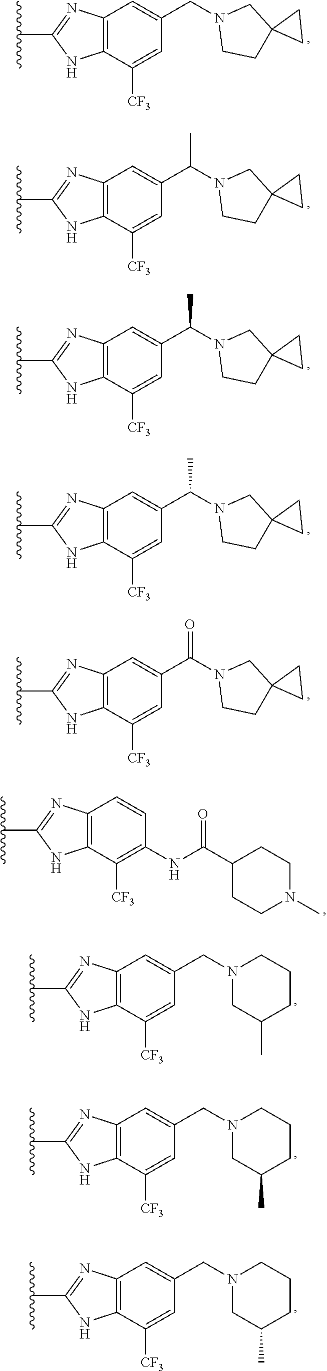

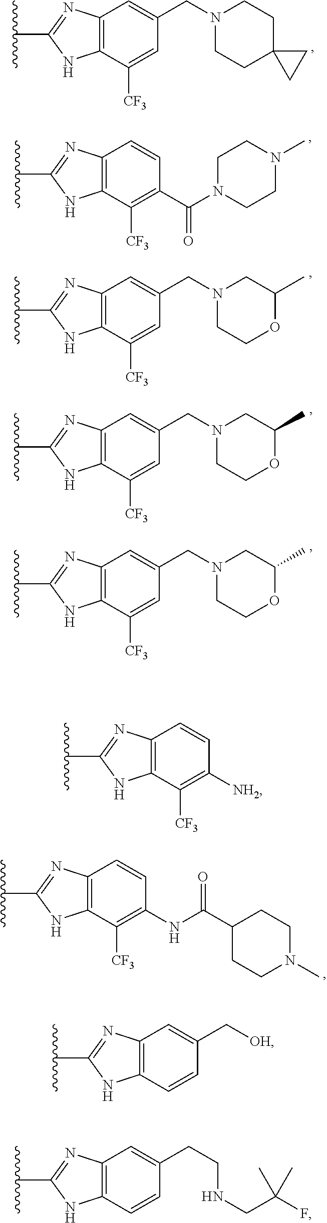

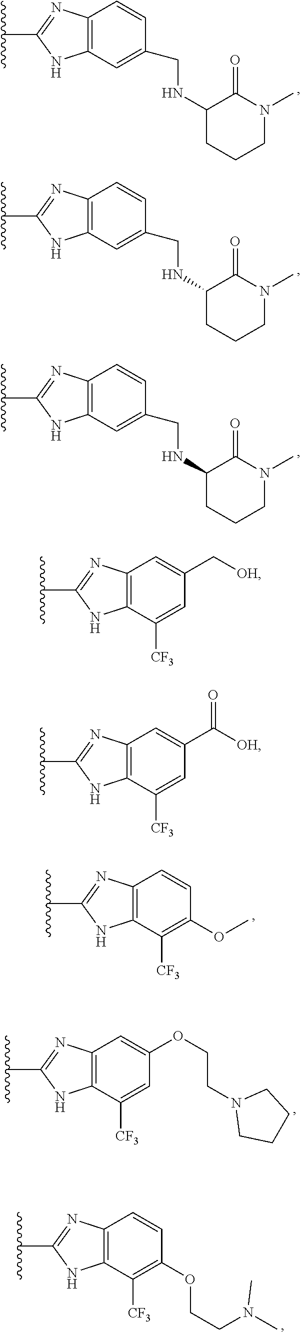

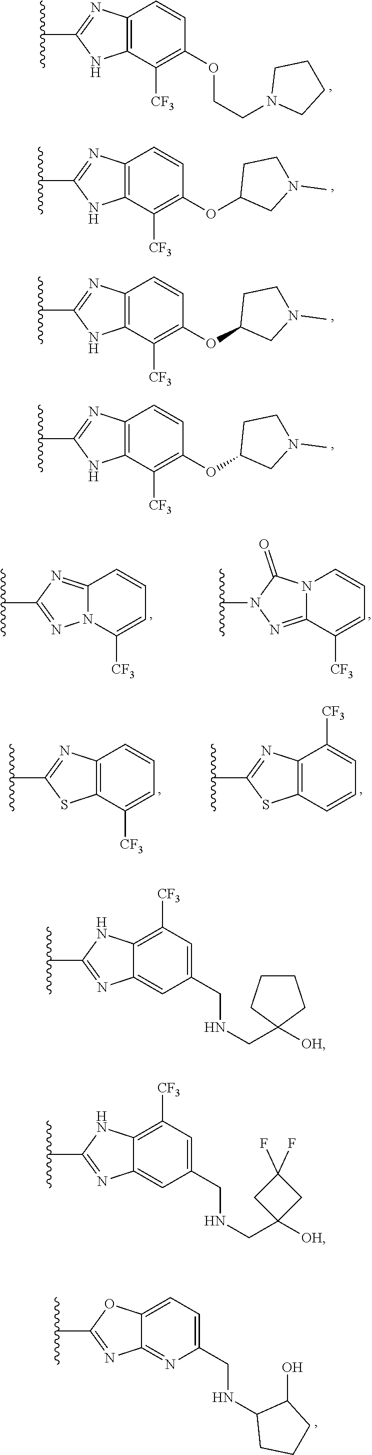

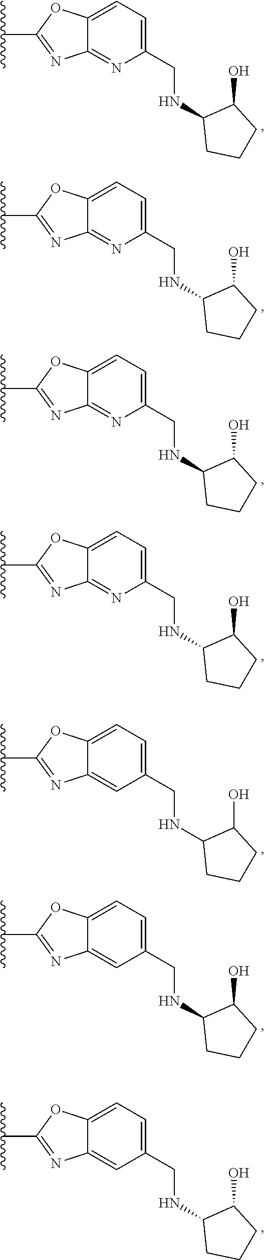

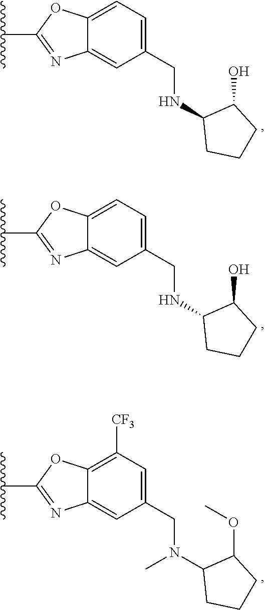 [SMILES: *c1nc2cc(CN[C@@H]3CCC[C@H]3O)ccc2o1 ; *c1nc2cc(CN[C@H]3CCC[C@@H]3O)ccc2o1 ; *c1nc2cc(CN(C)C3CCCC3OC)cc(C(F)(F)F)c2o1]

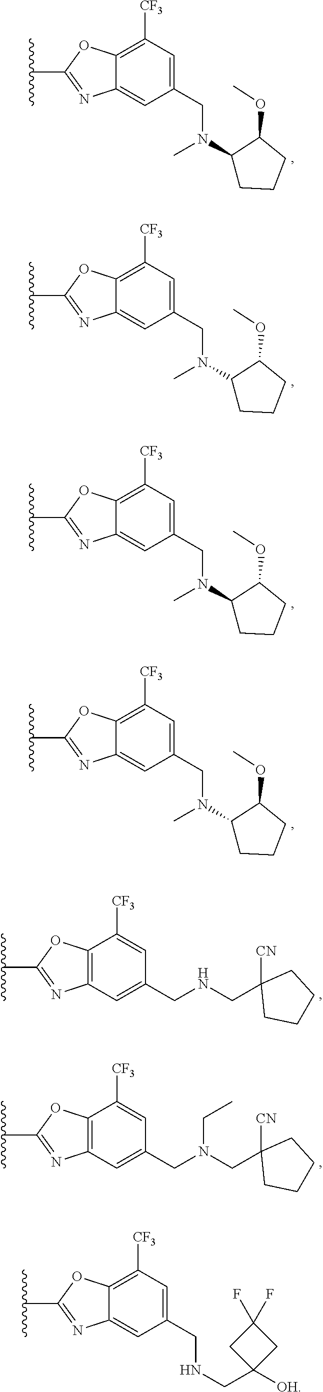

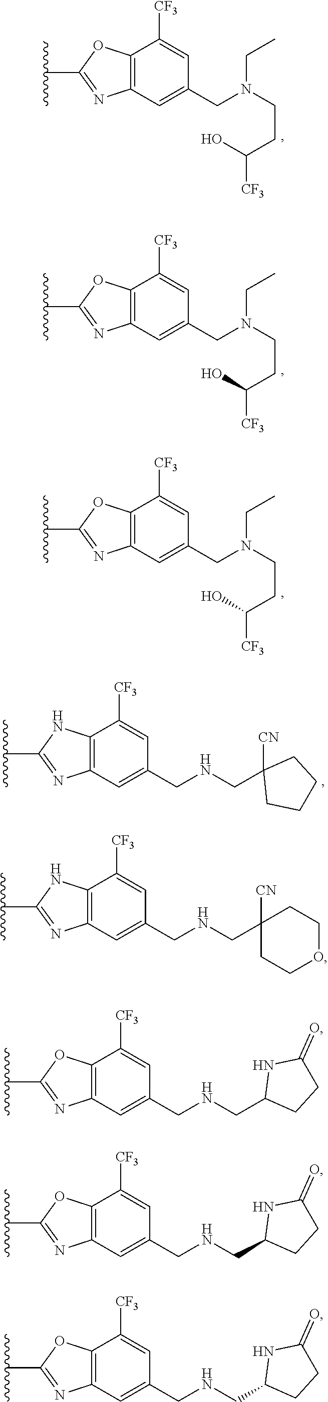

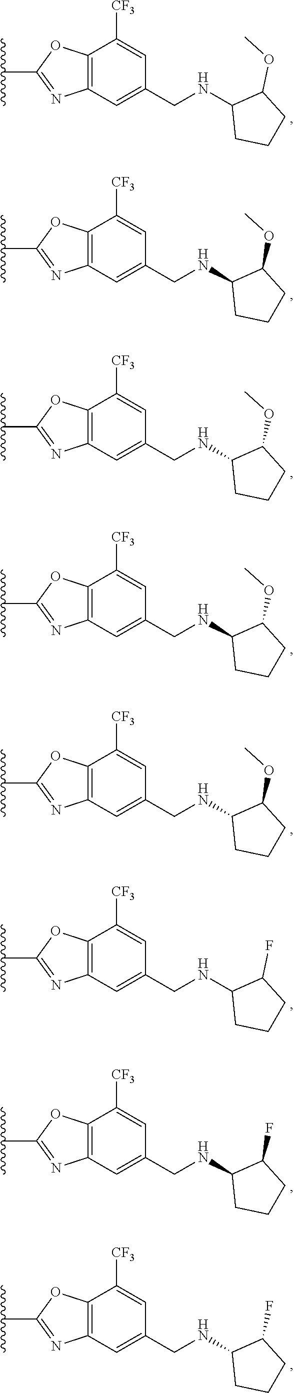

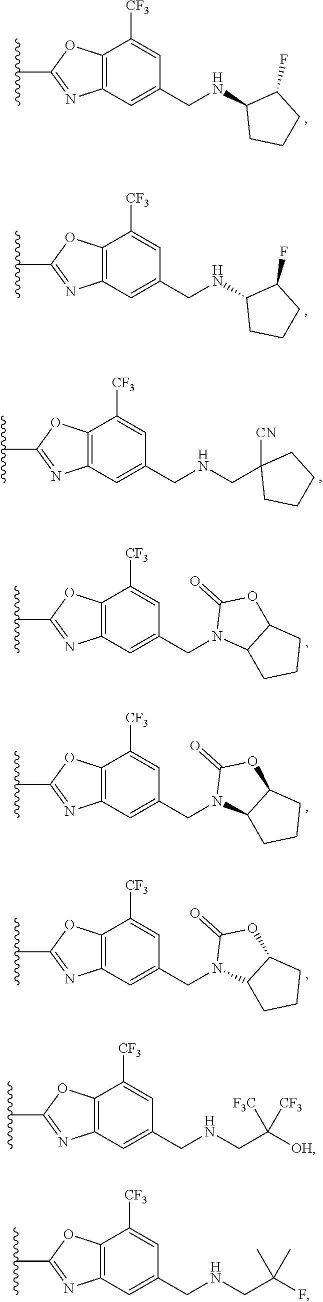

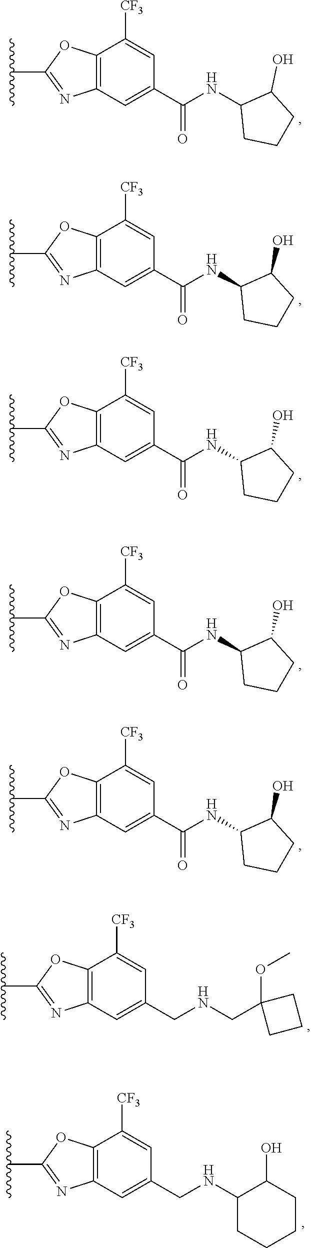

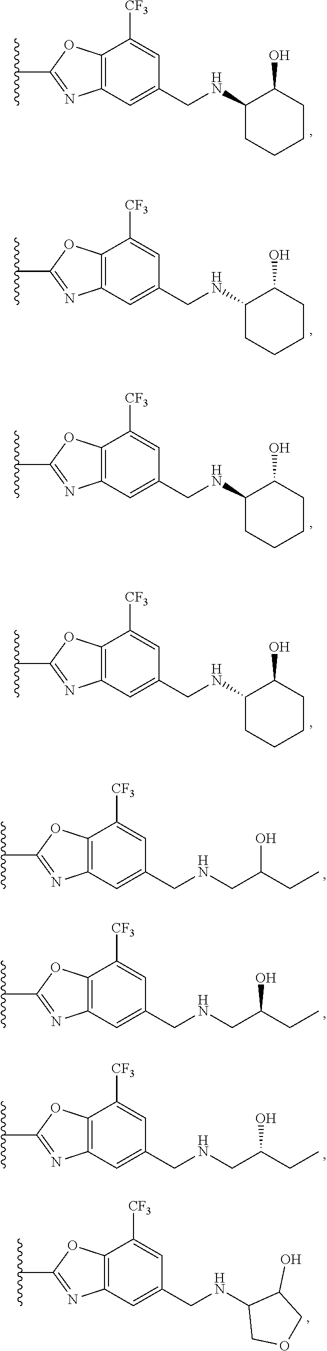

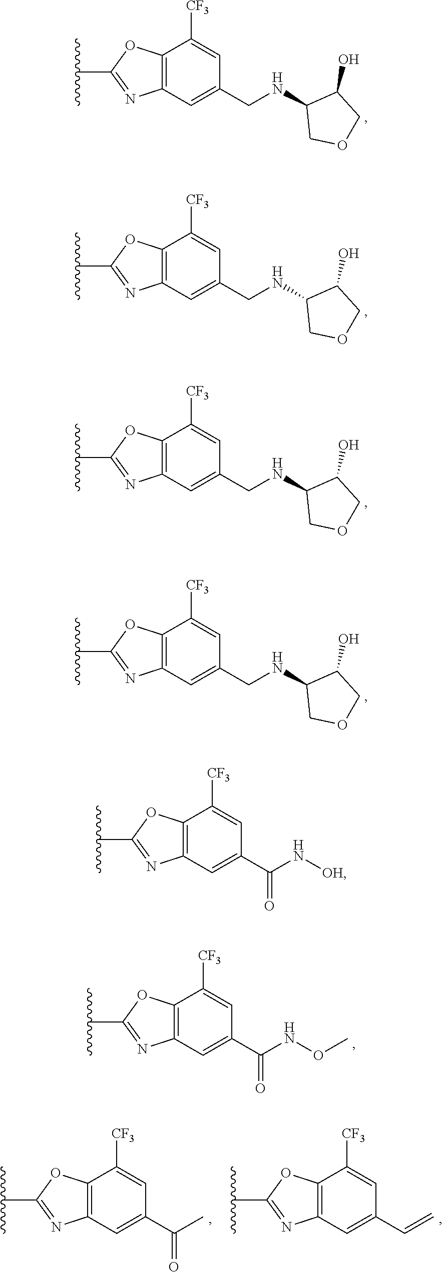

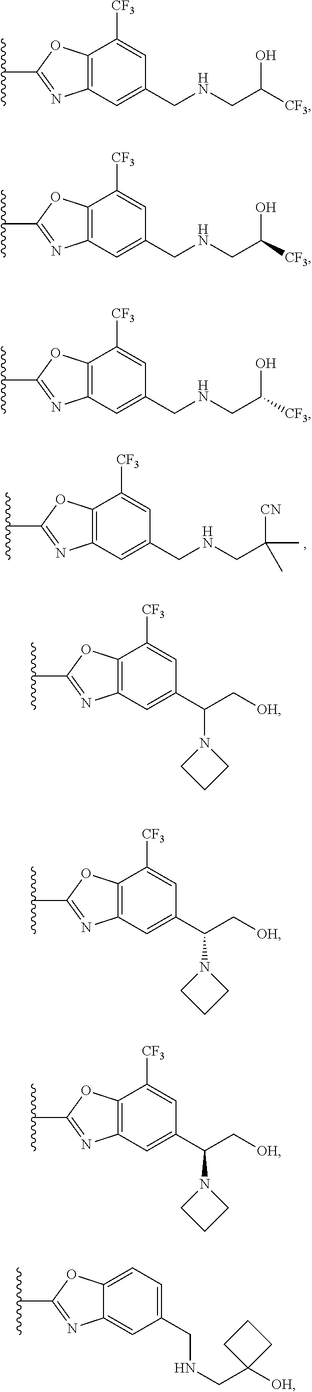

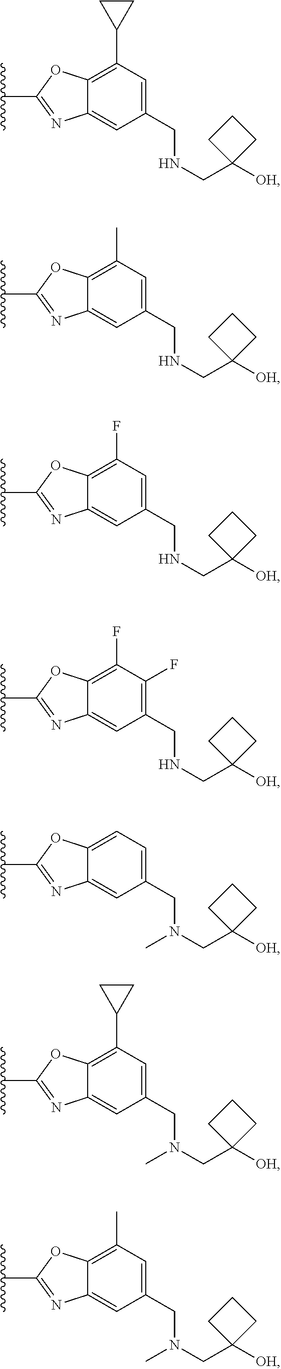

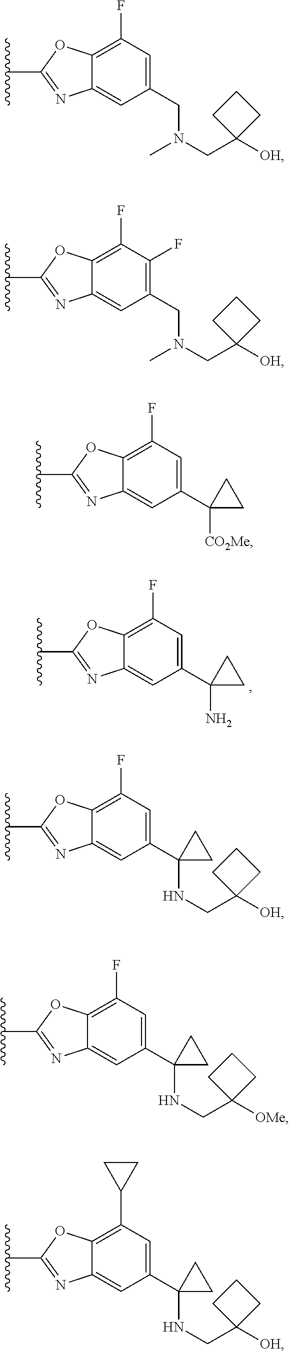

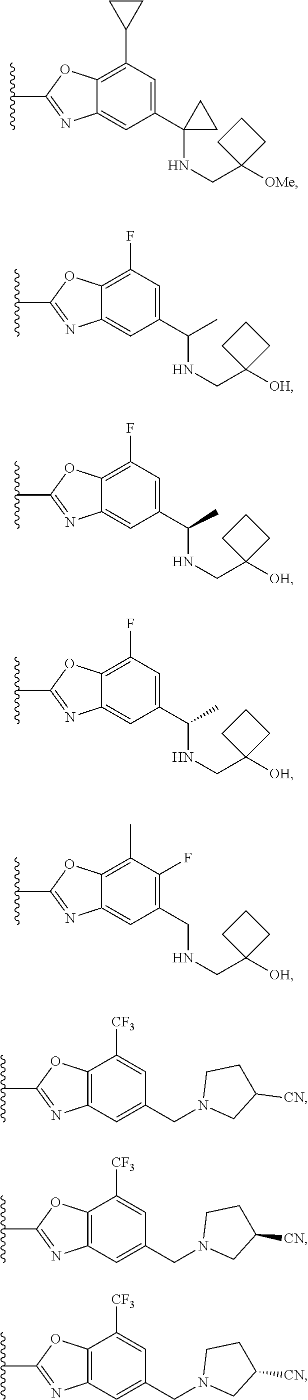

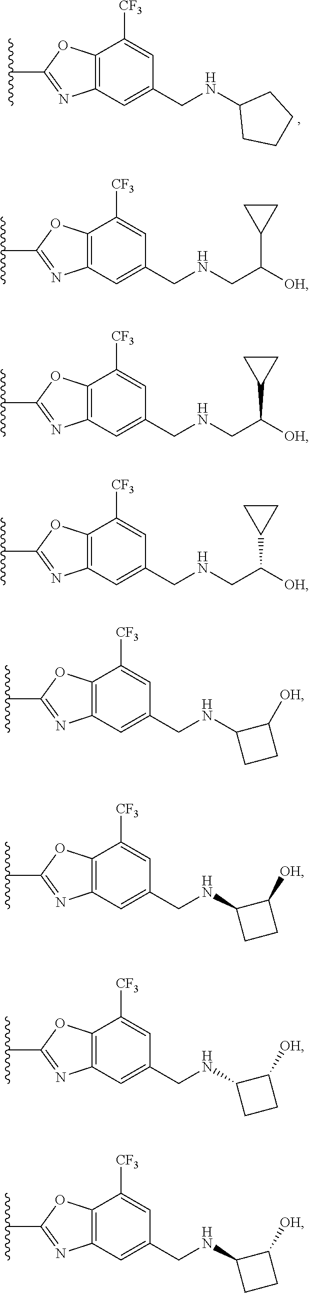

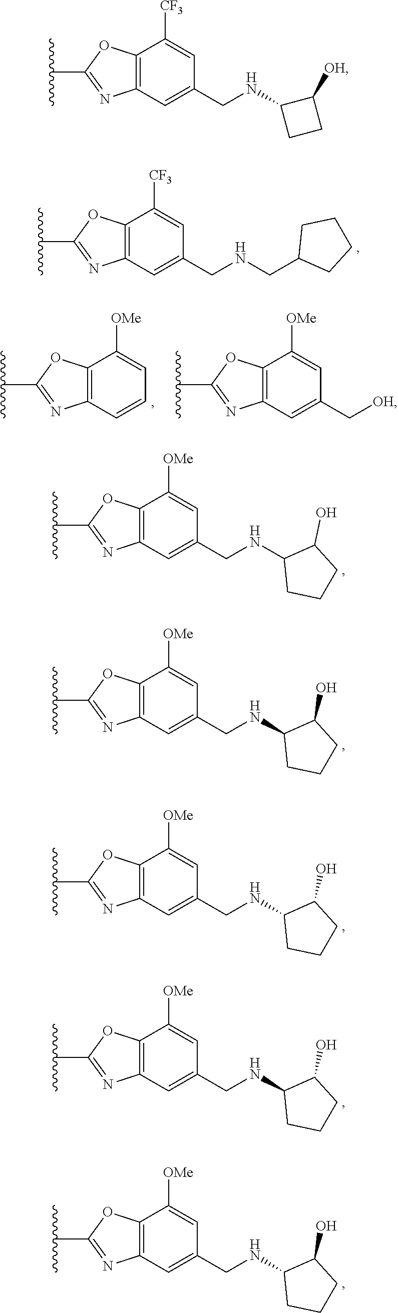

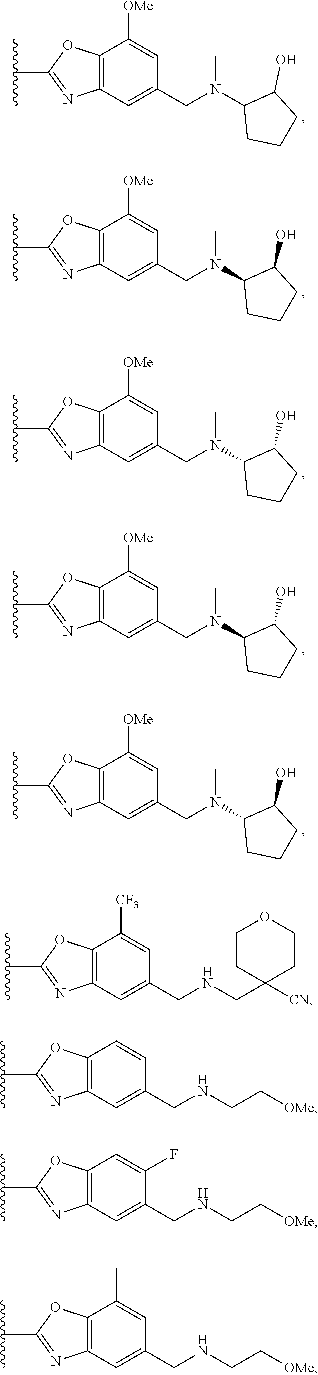

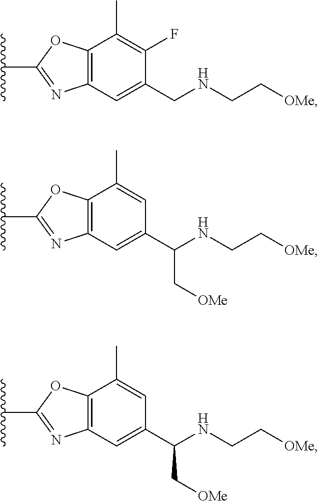 [SMILES: *c1nc2cc(CNCCOC)c(F)c(C)c2o1 ; *c1nc2cc(C(COC)NCCOC)cc(C)c2o1 ; *c1nc2cc([C@H](COC)NCCOC)cc(C)c2o1]

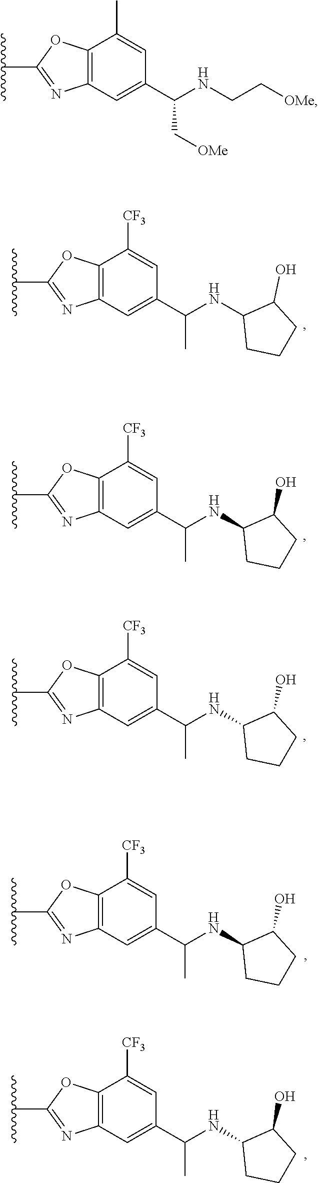 [SMILES: *c1nc2cc([C@@H](COC)NCCOC)cc(C)c2o1 ; *c1nc2cc(C(C)NC3CCCC3O)cc(C(F)(F)F)c2o1 ; *c1nc2cc(C(C)N[C@@H]3CCC[C@@H]3O)cc(C(F)(F)F)c2o1 ; *c1nc2cc(C(C)N[C@H]3CCC[C@H]3O)cc(C(F)(F)F)c2o1 ; *c1nc2cc(C(C)N[C@@H]3CCC[C@H]3O)cc(C(F)(F)F)c2o1 ; *c1nc2cc(C(C)N[C@H]3CCC[C@@H]3O)cc(C(F)(F)F)c2o1]

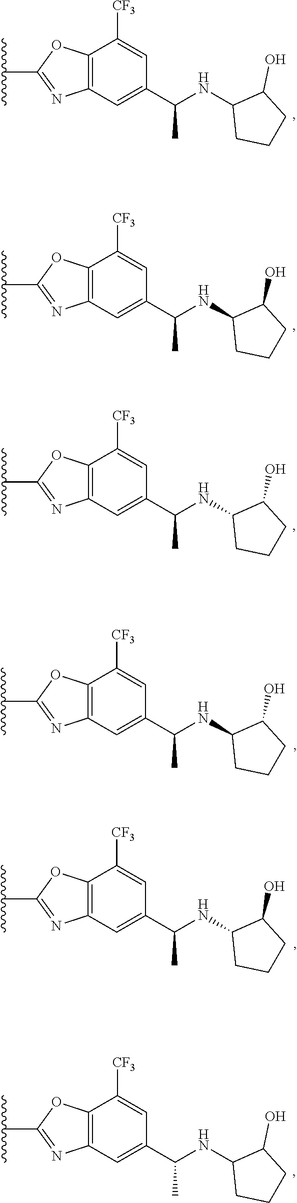 [SMILES: *c1nc2cc([C@H](C)NC3CCCC3O)cc(C(F)(F)F)c2o1 ; *c1nc2cc([C@H](C)N[C@@H]3CCC[C@@H]3O)cc(C(F)(F)F)c2o1 ; *c1nc2cc([C@H](C)N[C@H]3CCC[C@H]3O)cc(C(F)(F)F)c2o1 ; *c1nc2cc([C@H](C)N[C@@H]3CCC[C@H]3O)cc(C(F)(F)F)c2o1 ; *c1nc2cc([C@H](C)N[C@H]3CCC[C@@H]3O)cc(C(F)(F)F)c2o1 ; *c1nc2cc([C@@H](C)NC3CCCC3O)cc(C(F)(F)F)c2o1]

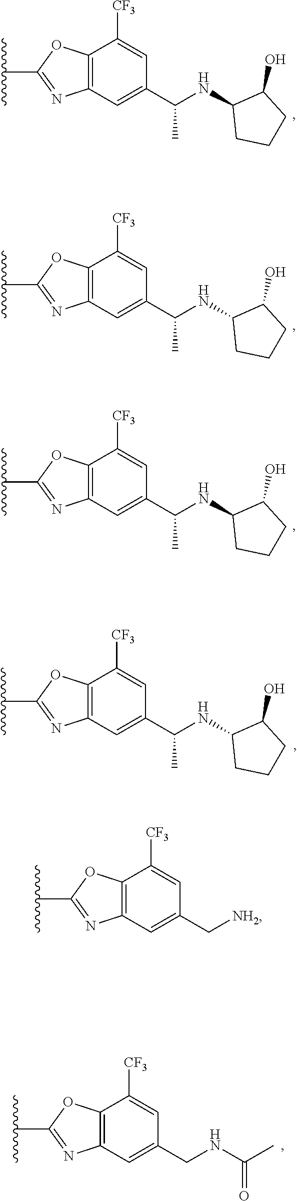 [SMILES: *c1nc2cc([C@@H](C)N[C@@H]3CCC[C@@H]3O)cc(C(F)(F)F)c2o1 ; *c1nc2cc([C@@H](C)N[C@H]3CCC[C@H]3O)cc(C(F)(F)F)c2o1 ; *c1nc2cc([C@@H](C)N[C@@H]3CCC[C@H]3O)cc(C(F)(F)F)c2o1 ; *c1nc2cc([C@@H](C)N[C@H]3CCC[C@@H]3O)cc(C(F)(F)F)c2o1 ; *c1nc2cc(CN)cc(C(F)(F)F)c2o1 ; *c1nc2cc(CNC(C)=O)cc(C(F)(F)F)c2o1]

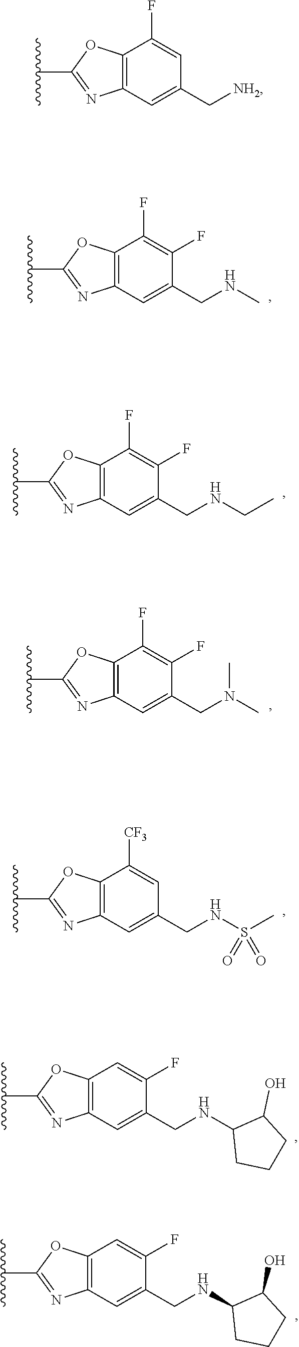

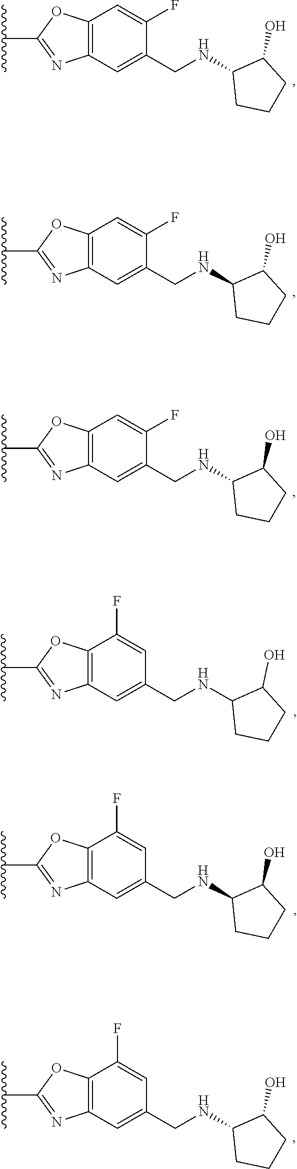 [SMILES: *c1nc2cc(CN[C@H]3CCC[C@H]3O)c(F)cc2o1 ; *c1nc2cc(CN[C@@H]3CCC[C@H]3O)c(F)cc2o1 ; *c1nc2cc(CN[C@H]3CCC[C@@H]3O)c(F)cc2o1 ; *c1nc2cc(CNC3CCCC3O)cc(F)c2o1 ; *c1nc2cc(CN[C@@H]3CCC[C@@H]3O)cc(F)c2o1 ; *c1nc2cc(CN[C@H]3CCC[C@H]3O)cc(F)c2o1]

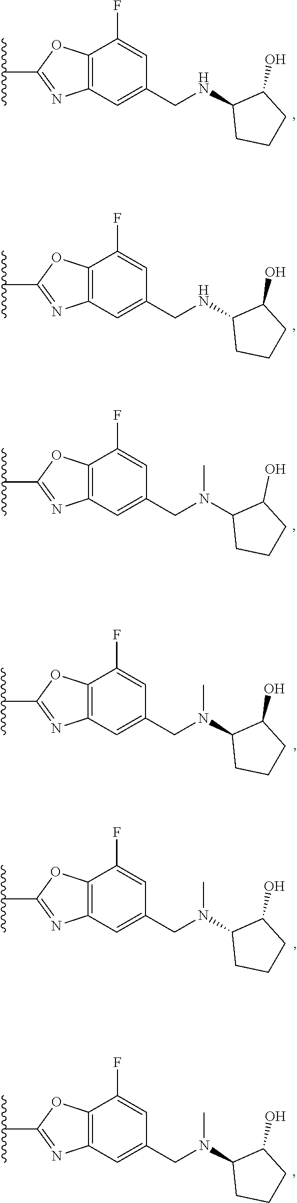 [SMILES: *c1nc2cc(CN[C@@H]3CCC[C@H]3O)cc(F)c2o1 ; *c1nc2cc(CN[C@H]3CCC[C@@H]3O)cc(F)c2o1 ; *c1nc2cc(CN(C)C3CCCC3O)cc(F)c2o1 ; *c1nc2cc(CN(C)[C@@H]3CCC[C@@H]3O)cc(F)c2o1 ; *c1nc2cc(CN(C)[C@H]3CCC[C@H]3O)cc(F)c2o1 ; *c1nc2cc(CN(C)[C@@H]3CCC[C@H]3O)cc(F)c2o1]

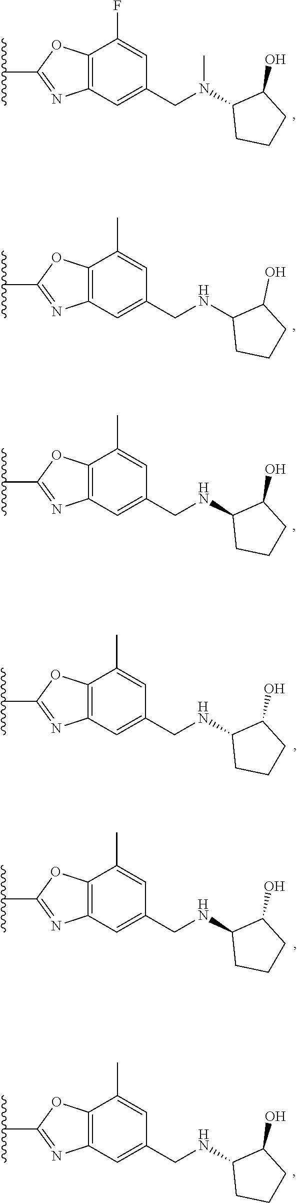 [SMILES: *c1nc2cc(CN(C)[C@H]3CCC[C@@H]3O)cc(F)c2o1 ; *c1nc2cc(CNC3CCCC3O)cc(C)c2o1 ; *c1nc2cc(CN[C@@H]3CCC[C@@H]3O)cc(C)c2o1 ; *c1nc2cc(CN[C@H]3CCC[C@H]3O)cc(C)c2o1 ; *c1nc2cc(CN[C@@H]3CCC[C@H]3O)cc(C)c2o1 ; *c1nc2cc(CN[C@H]3CCC[C@@H]3O)cc(C)c2o1]

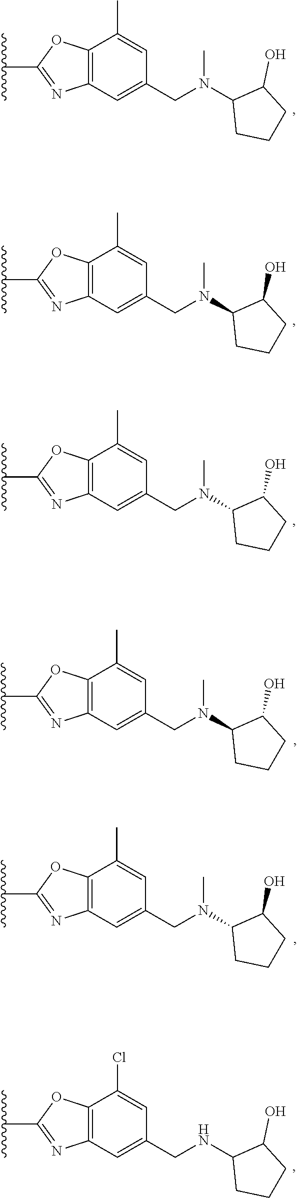 [SMILES: *c1nc2cc(CN(C)C3CCCC3O)cc(C)c2o1 ; *c1nc2cc(CN(C)[C@@H]3CCC[C@@H]3O)cc(C)c2o1 ; *c1nc2cc(CN(C)[C@H]3CCC[C@H]3O)cc(C)c2o1 ; *c1nc2cc(CN(C)[C@@H]3CCC[C@H]3O)cc(C)c2o1 ; *c1nc2cc(CN(C)[C@H]3CCC[C@@H]3O)cc(C)c2o1 ; *c1nc2cc(CNC3CCCC3O)cc(Cl)c2o1]

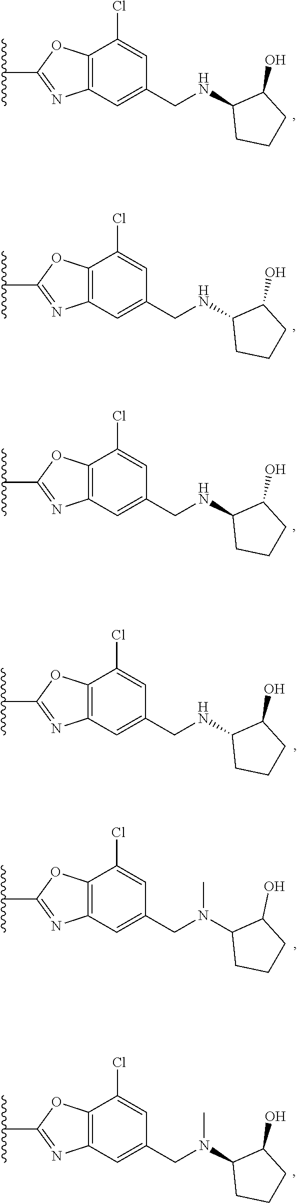 [SMILES: *c1nc2cc(CN[C@@H]3CCC[C@@H]3O)cc(Cl)c2o1 ; *c1nc2cc(CN[C@H]3CCC[C@H]3O)cc(Cl)c2o1 ; *c1nc2cc(CN[C@@H]3CCC[C@H]3O)cc(Cl)c2o1 ; *c1nc2cc(CN[C@H]3CCC[C@@H]3O)cc(Cl)c2o1 ; *c1nc2cc(CN(C)C3CCCC3O)cc(Cl)c2o1 ; *c1nc2cc(CN(C)[C@@H]3CCC[C@@H]3O)cc(Cl)c2o1]

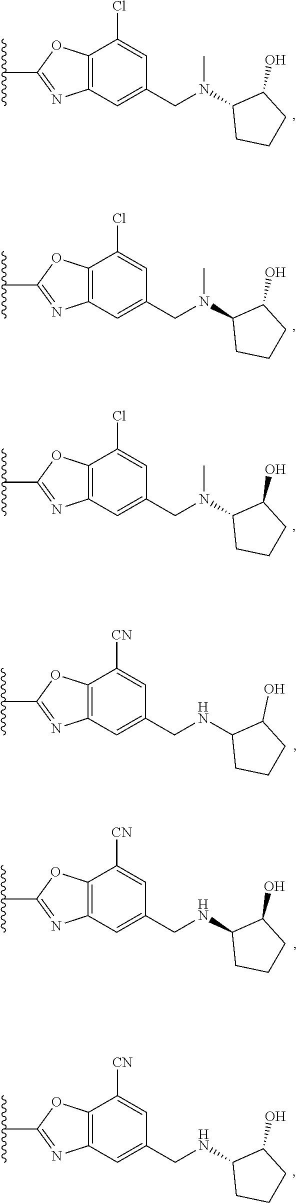 [SMILES: *c1nc2cc(CN(C)[C@H]3CCC[C@H]3O)cc(Cl)c2o1 ; *c1nc2cc(CN(C)[C@@H]3CCC[C@H]3O)cc(Cl)c2o1 ; *c1nc2cc(CN(C)[C@H]3CCC[C@@H]3O)cc(Cl)c2o1 ; *c1nc2cc(CNC3CCCC3O)cc(C#N)c2o1 ; *c1nc2cc(CN[C@@H]3CCC[C@@H]3O)cc(C#N)c2o1 ; *c1nc2cc(CN[C@H]3CCC[C@H]3O)cc(C#N)c2o1]

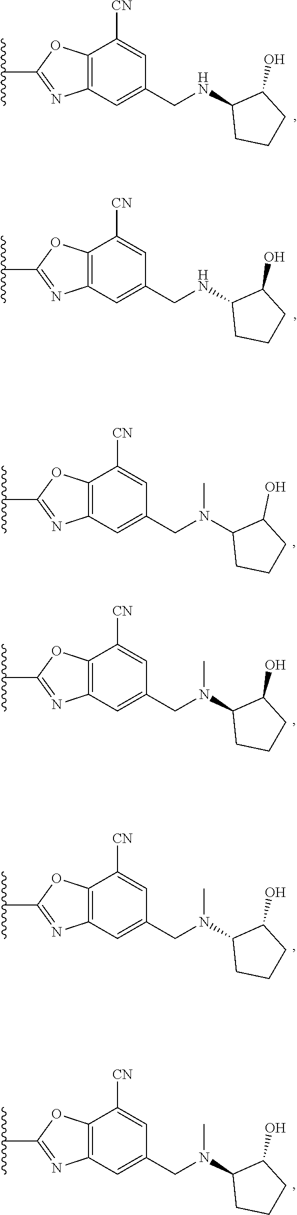 [SMILES: *c1nc2cc(CN[C@@H]3CCC[C@H]3O)cc(C#N)c2o1 ; *c1nc2cc(CN[C@H]3CCC[C@@H]3O)cc(C#N)c2o1 ; *c1nc2cc(CN(C)C3CCCC3O)cc(C#N)c2o1 ; *c1nc2cc(CN(C)[C@@H]3CCC[C@@H]3O)cc(C#N)c2o1 ; *c1nc2cc(CN(C)[C@H]3CCC[C@H]3O)cc(C#N)c2o1 ; *c1nc2cc(CN(C)[C@@H]3CCC[C@H]3O)cc(C#N)c2o1]

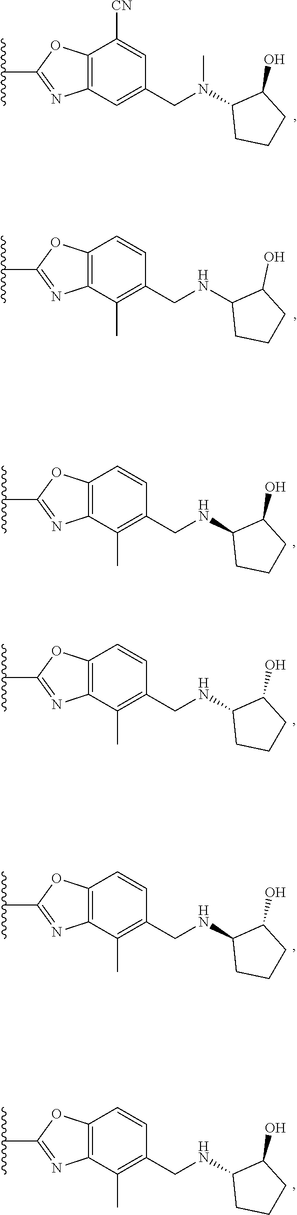 [SMILES: *c1nc2cc(CN(C)[C@H]3CCC[C@@H]3O)cc(C#N)c2o1 ; *c1nc2c(C)c(CNC3CCCC3O)ccc2o1 ; *c1nc2c(C)c(CN[C@@H]3CCC[C@@H]3O)ccc2o1 ; *c1nc2c(C)c(CN[C@H]3CCC[C@H]3O)ccc2o1 ; *c1nc2c(C)c(CN[C@@H]3CCC[C@H]3O)ccc2o1 ; *c1nc2c(C)c(CN[C@H]3CCC[C@@H]3O)ccc2o1]

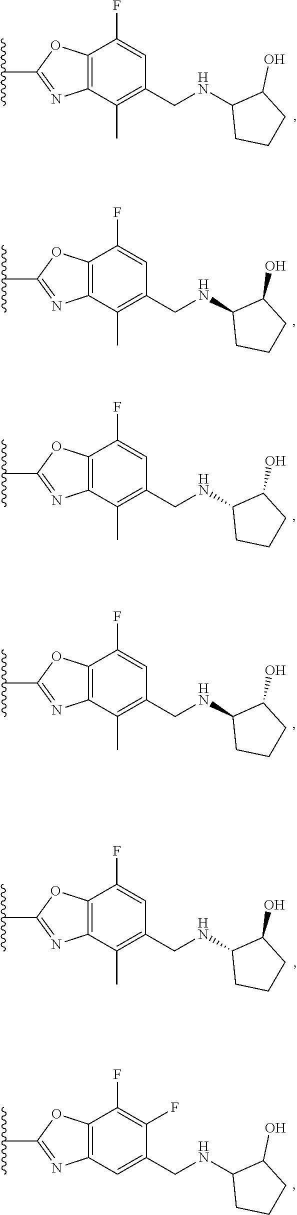 [SMILES: *c1nc2c(C)c(CNC3CCCC3O)cc(F)c2o1 ; *c1nc2c(C)c(CN[C@@H]3CCC[C@@H]3O)cc(F)c2o1 ; *c1nc2c(C)c(CN[C@H]3CCC[C@H]3O)cc(F)c2o1 ; *c1nc2c(C)c(CN[C@@H]3CCC[C@H]3O)cc(F)c2o1 ; *c1nc2c(C)c(CN[C@H]3CCC[C@@H]3O)cc(F)c2o1 ; *c1nc2cc(CNC3CCCC3O)c(F)c(F)c2o1]

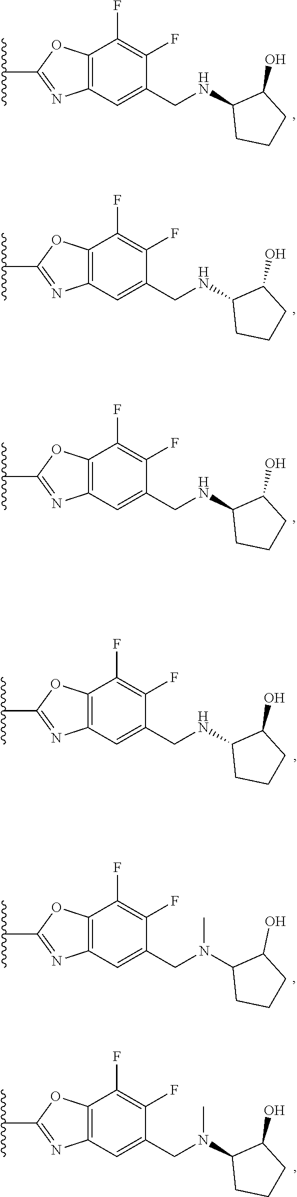 [SMILES: *c1nc2cc(CN[C@@H]3CCC[C@@H]3O)c(F)c(F)c2o1 ; *c1nc2cc(CN[C@H]3CCC[C@H]3O)c(F)c(F)c2o1 ; *c1nc2cc(CN[C@@H]3CCC[C@H]3O)c(F)c(F)c2o1 ; *c1nc2cc(CN[C@H]3CCC[C@@H]3O)c(F)c(F)c2o1 ; *c1nc2cc(CN(C)C3CCCC3O)c(F)c(F)c2o1 ; *c1nc2cc(CN(C)[C@@H]3CCC[C@@H]3O)c(F)c(F)c2o1]

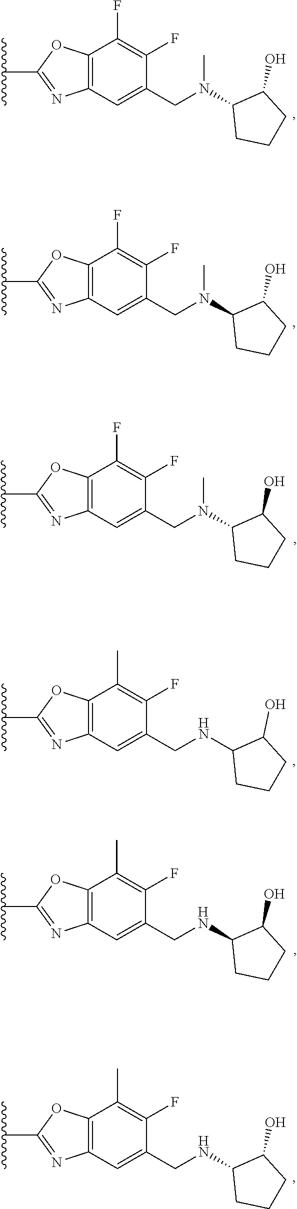 [SMILES: *c1nc2cc(CN(C)[C@H]3CCC[C@H]3O)c(F)c(F)c2o1 ; *c1nc2cc(CN(C)[C@@H]3CCC[C@H]3O)c(F)c(F)c2o1 ; *c1nc2cc(CN(C)[C@H]3CCC[C@@H]3O)c(F)c(F)c2o1 ; *c1nc2cc(CNC3CCCC3O)c(F)c(C)c2o1 ; *c1nc2cc(CN[C@@H]3CCC[C@@H]3O)c(F)c(C)c2o1 ; *c1nc2cc(CN[C@H]3CCC[C@H]3O)c(F)c(C)c2o1]

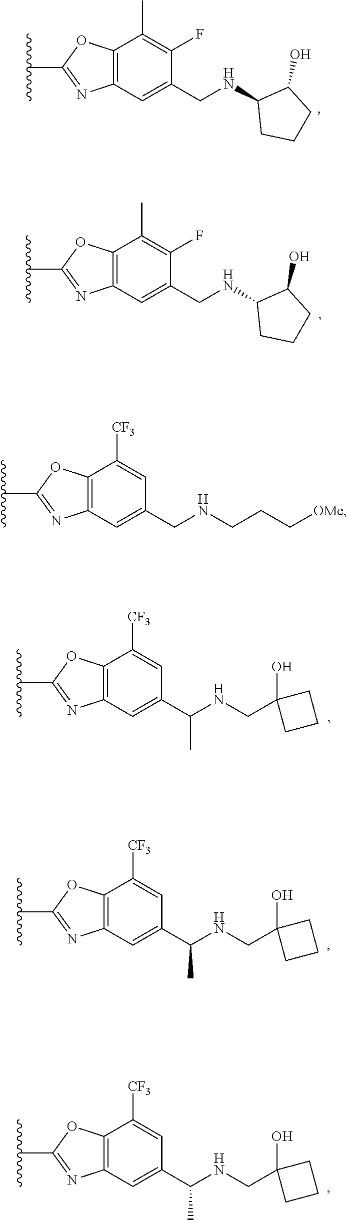 [SMILES: *c1nc2cc(CN[C@@H]3CCC[C@H]3O)c(F)c(C)c2o1 ; *c1nc2cc(CN[C@H]3CCC[C@@H]3O)c(F)c(C)c2o1 ; *c1nc2cc(CNCCCOC)cc(C(F)(F)F)c2o1 ; *c1nc2cc(C(C)NCC3(O)CCC3)cc(C(F)(F)F)c2o1 ; *c1nc2cc([C@H](C)NCC3(O)CCC3)cc(C(F)(F)F)c2o1 ; *c1nc2cc([C@@H](C)NCC3(O)CCC3)cc(C(F)(F)F)c2o1]

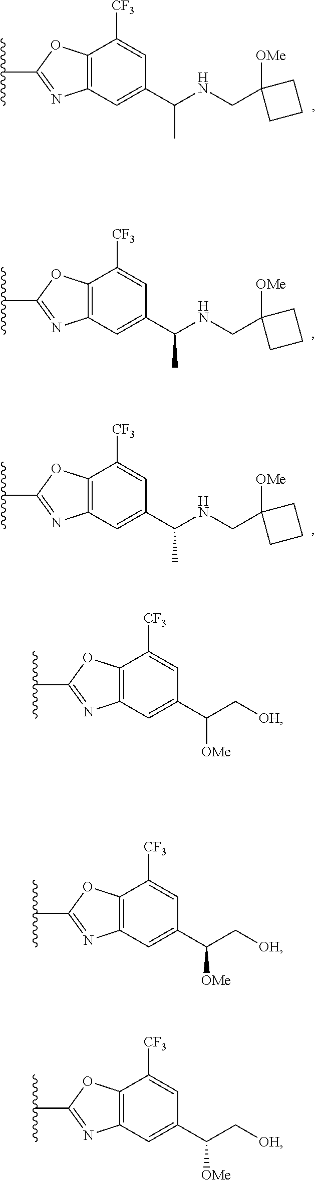 [SMILES: *c1nc2cc(C(C)NCC3(OC)CCC3)cc(C(F)(F)F)c2o1 ; *c1nc2cc([C@H](C)NCC3(OC)CCC3)cc(C(F)(F)F)c2o1 ; *c1nc2cc([C@@H](C)NCC3(OC)CCC3)cc(C(F)(F)F)c2o1 ; *c1nc2cc(C(CO)OC)cc(C(F)(F)F)c2o1 ; *c1nc2cc([C@@H](CO)OC)cc(C(F)(F)F)c2o1 ; *c1nc2cc([C@H](CO)OC)cc(C(F)(F)F)c2o1]

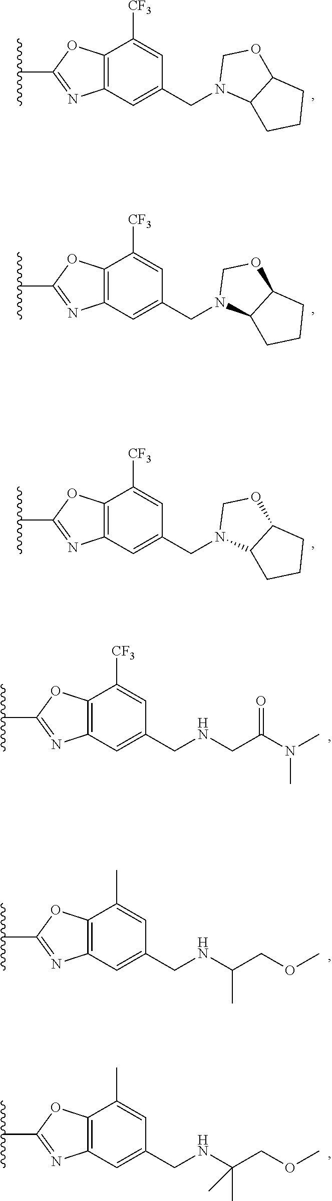

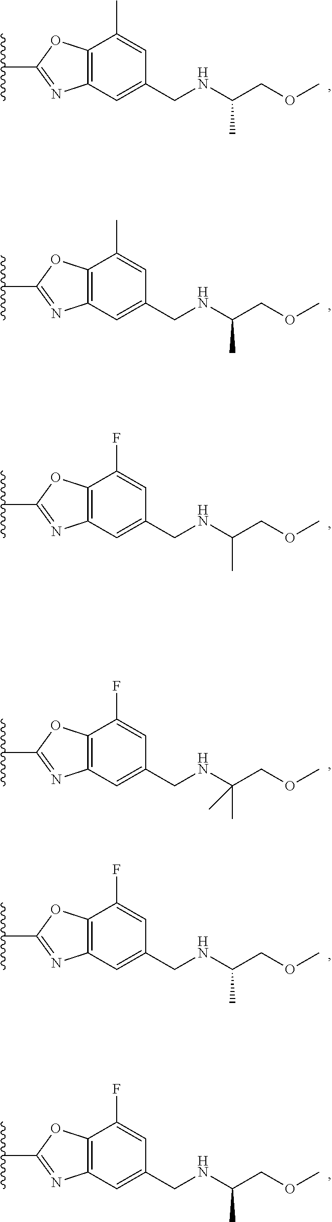 [SMILES: *c1nc2cc(CN[C@@H](C)COC)cc(C)c2o1 ; *c1nc2cc(CN[C@H](C)COC)cc(C)c2o1 ; *c1nc2cc(CNC(C)COC)cc(F)c2o1 ; *c1nc2cc(CNC(C)(C)COC)cc(F)c2o1 ; *c1nc2cc(CN[C@@H](C)COC)cc(F)c2o1 ; *c1nc2cc(CN[C@H](C)COC)cc(F)c2o1]

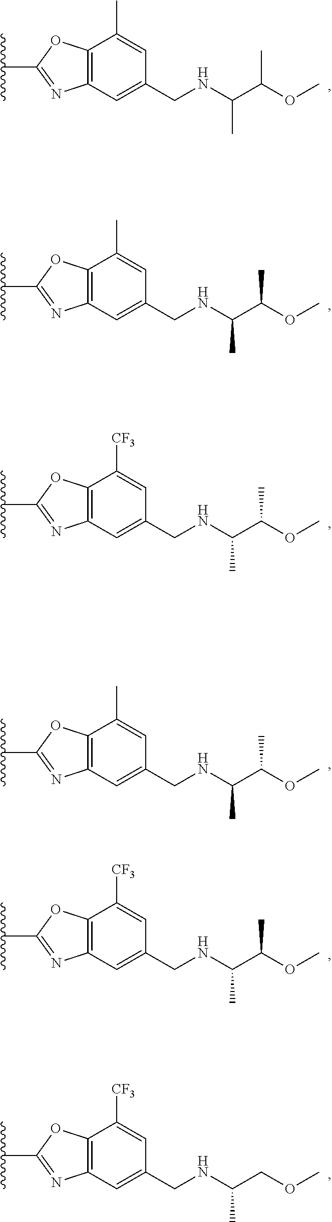 [SMILES: *c1nc2cc(CNC(C)C(C)OC)cc(C)c2o1 ; *c1nc2cc(CN[C@H](C)[C@@H](C)OC)cc(C)c2o1 ; *c1nc2cc(CN[C@@H](C)[C@H](C)OC)cc(C(F)(F)F)c2o1 ; *c1nc2cc(CN[C@H](C)[C@H](C)OC)cc(C)c2o1 ; *c1nc2cc(CN[C@@H](C)[C@@H](C)OC)cc(C(F)(F)F)c2o1 ; *c1nc2cc(CN[C@@H](C)COC)cc(C(F)(F)F)c2o1]

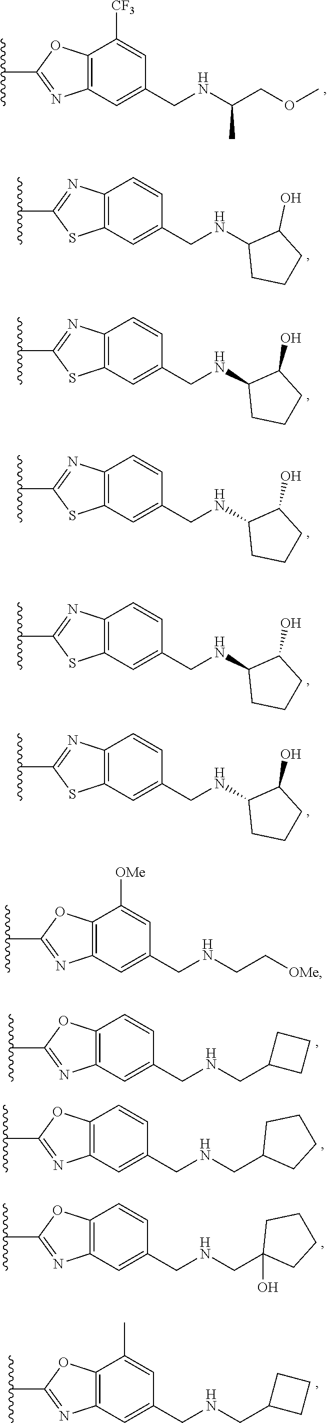

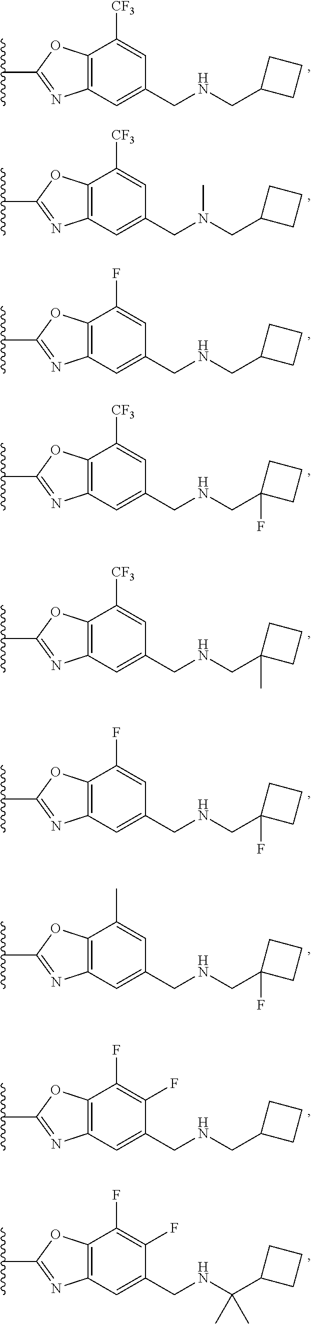

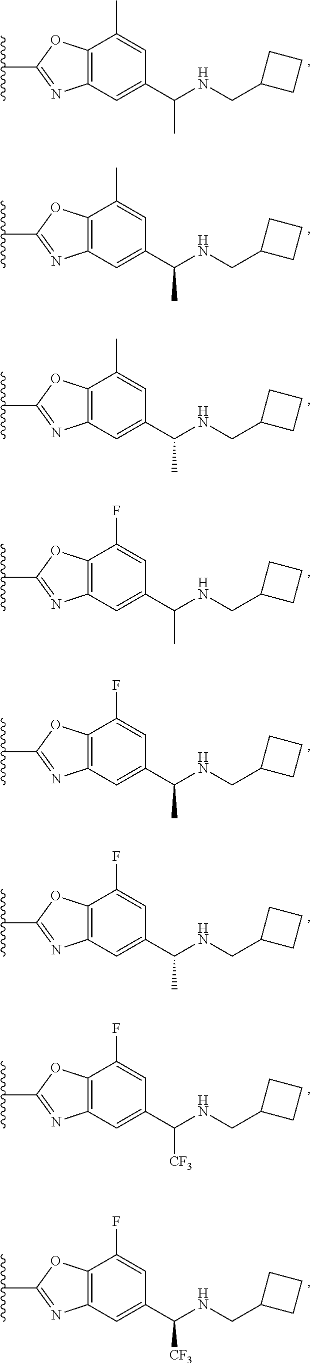

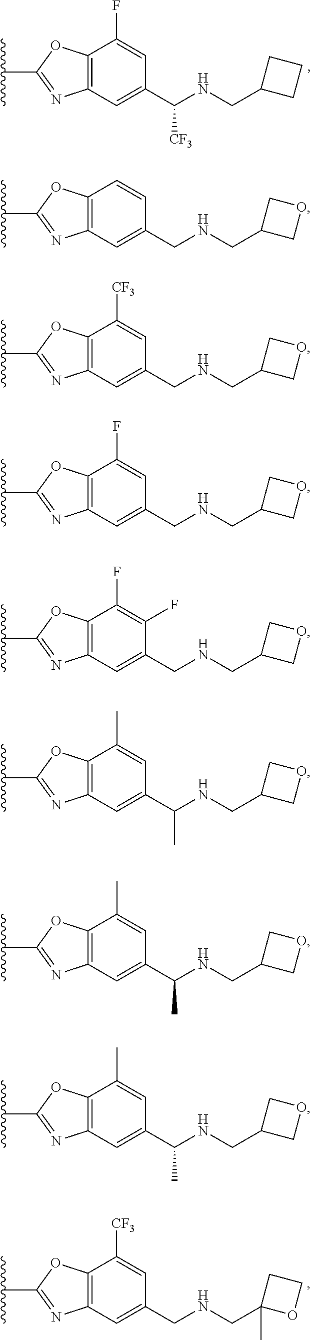

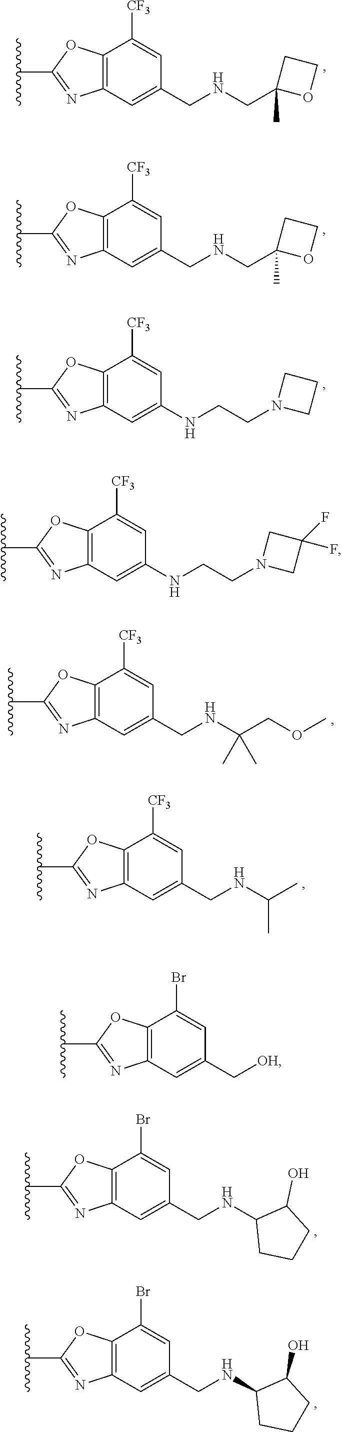

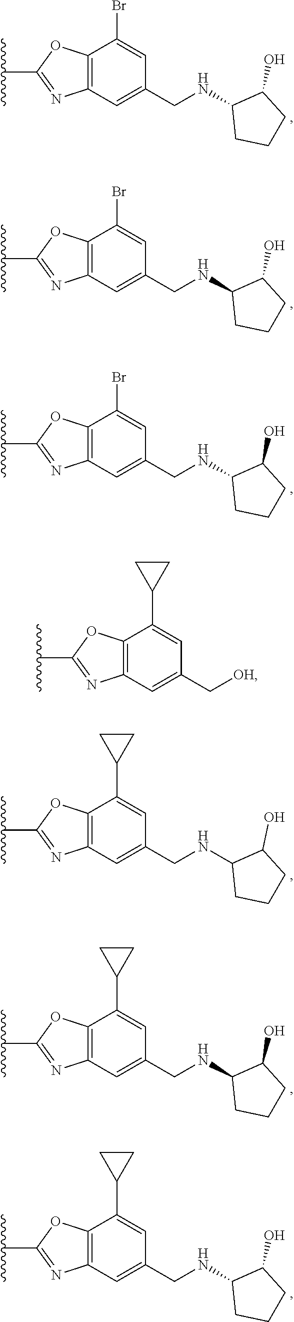

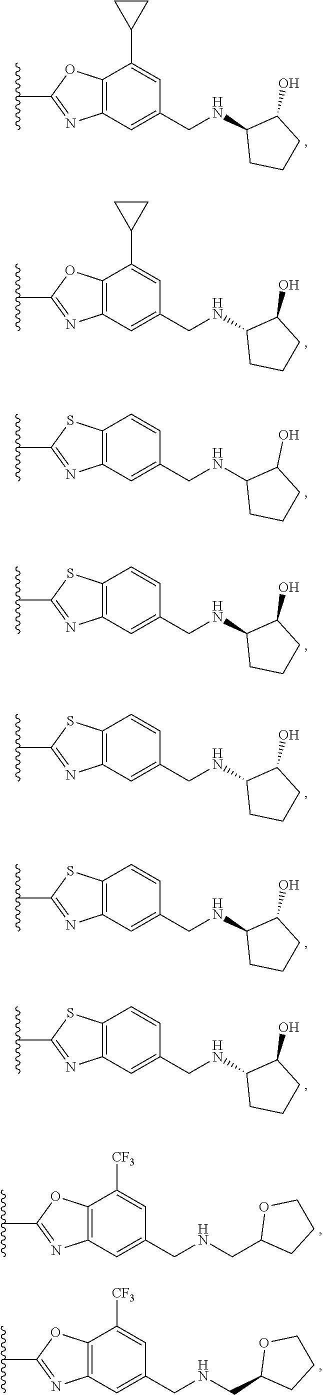

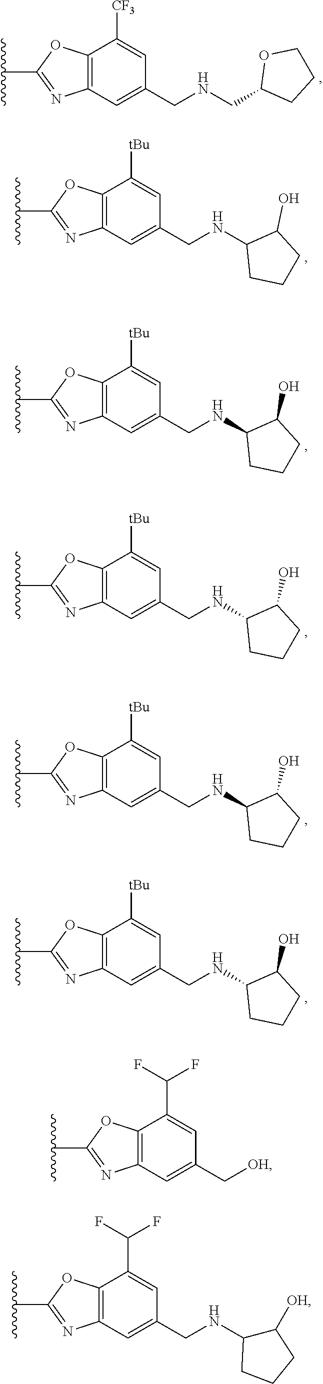

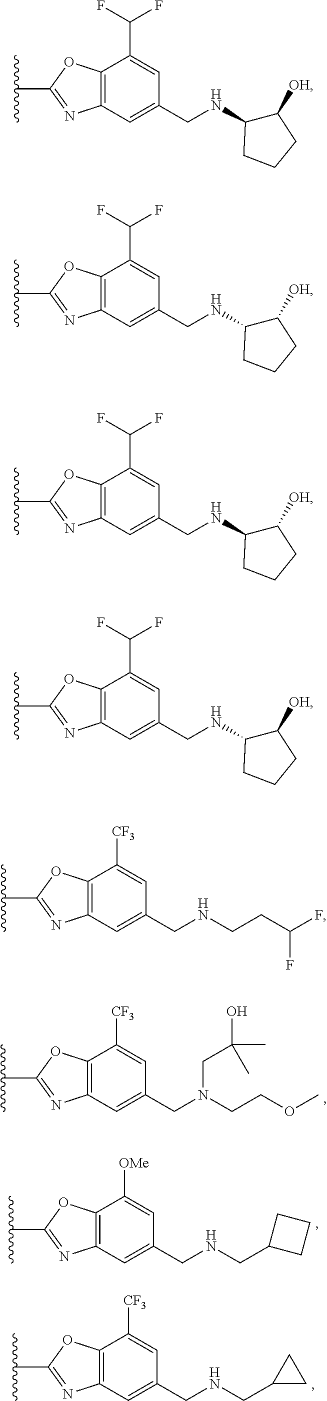

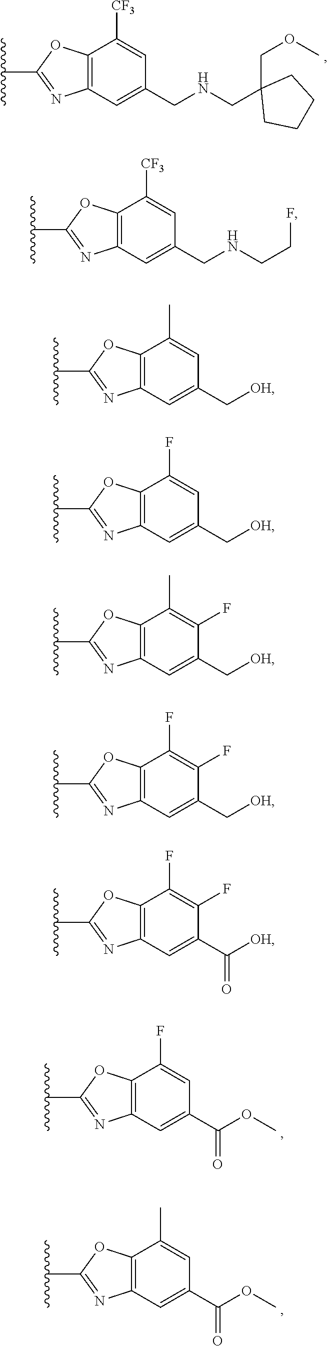

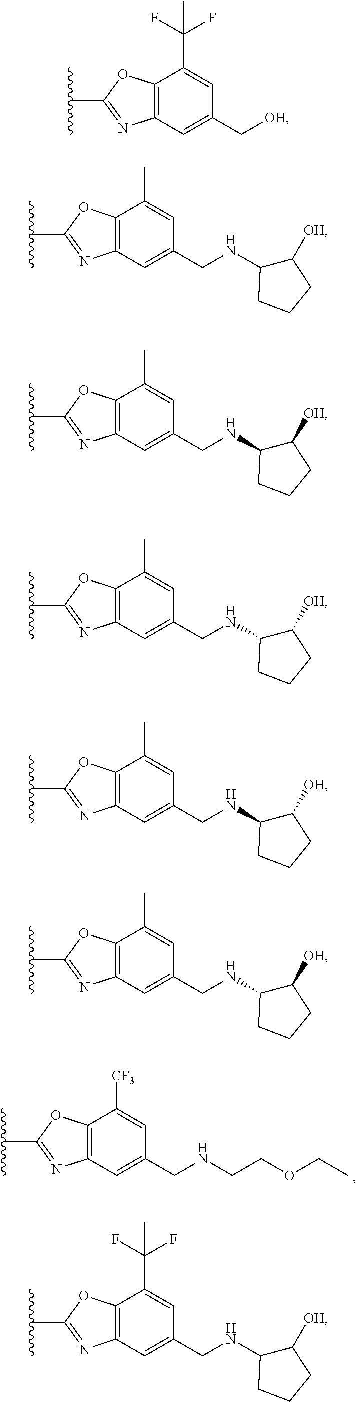

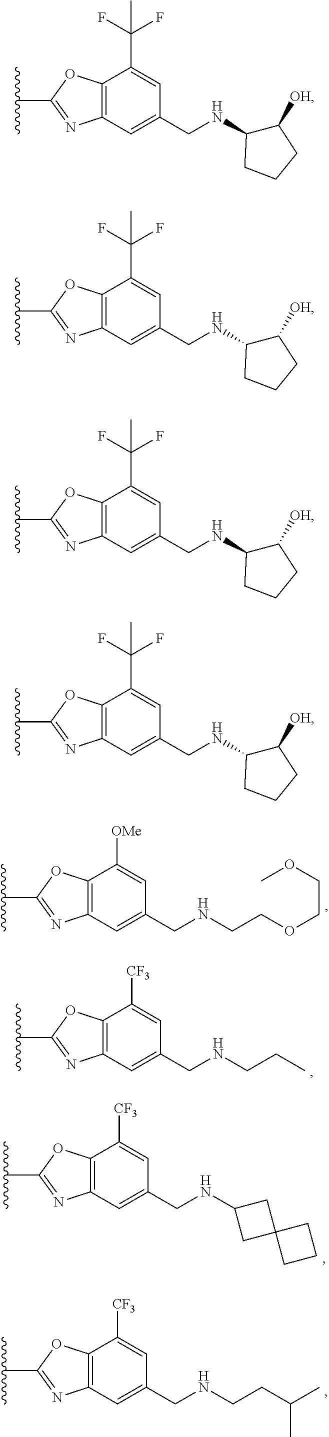

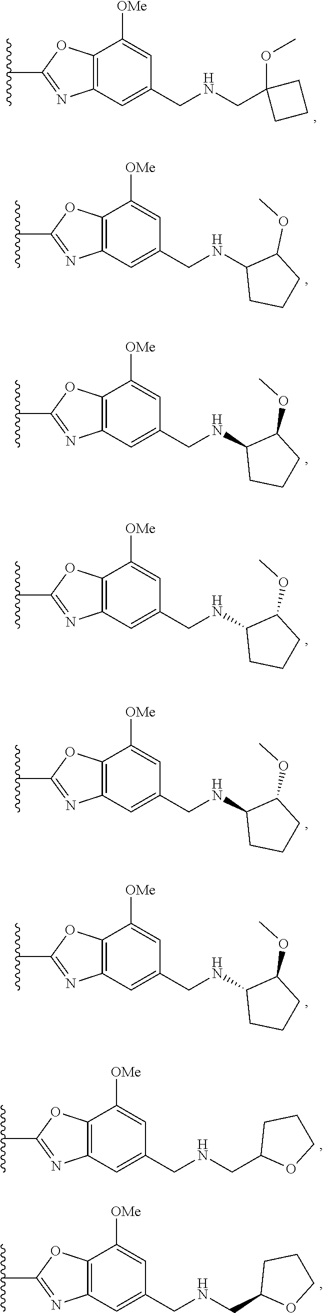

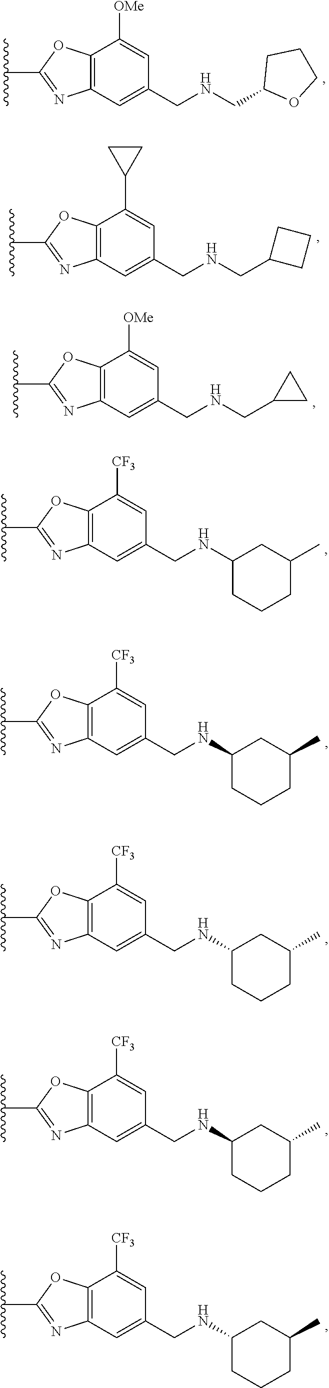

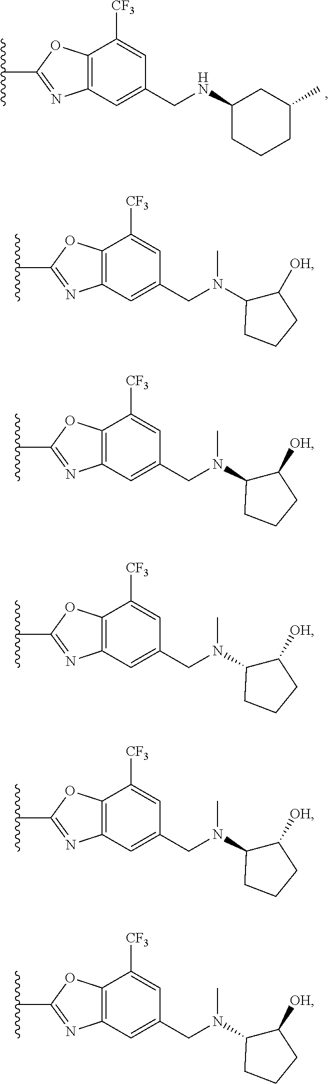 [SMILES: *c1nc2cc(CN[C@@H]3CCC[C@@H](C)C3)cc(C(F)(F)F)c2o1 ; *c1nc2cc(CN(C)C3CCCC3O)cc(C(F)(F)F)c2o1 ; *c1nc2cc(CN(C)[C@@H]3CCC[C@@H]3O)cc(C(F)(F)F)c2o1 ; *c1nc2cc(CN(C)[C@H]3CCC[C@H]3O)cc(C(F)(F)F)c2o1 ; *c1nc2cc(CN(C)[C@@H]3CCC[C@H]3O)cc(C(F)(F)F)c2o1 ; *c1nc2cc(CN(C)[C@H]3CCC[C@@H]3O)cc(C(F)(F)F)c2o1]

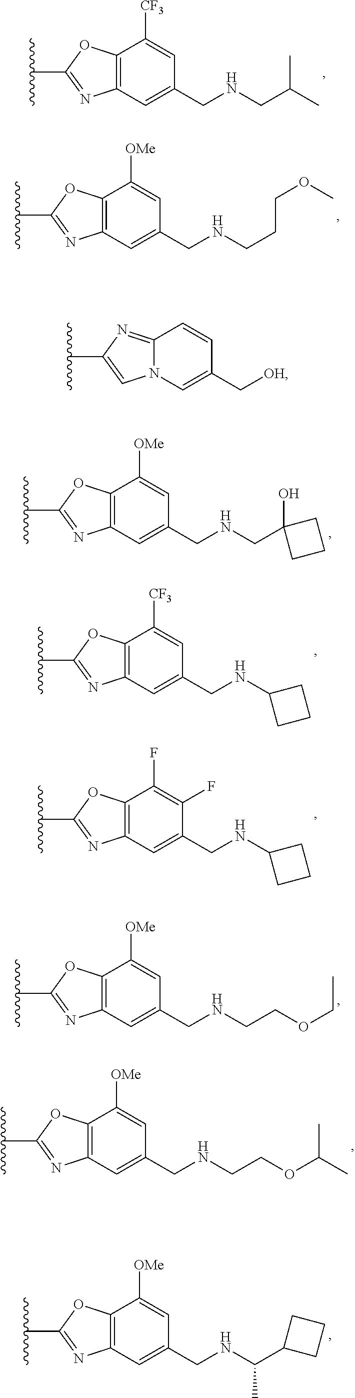

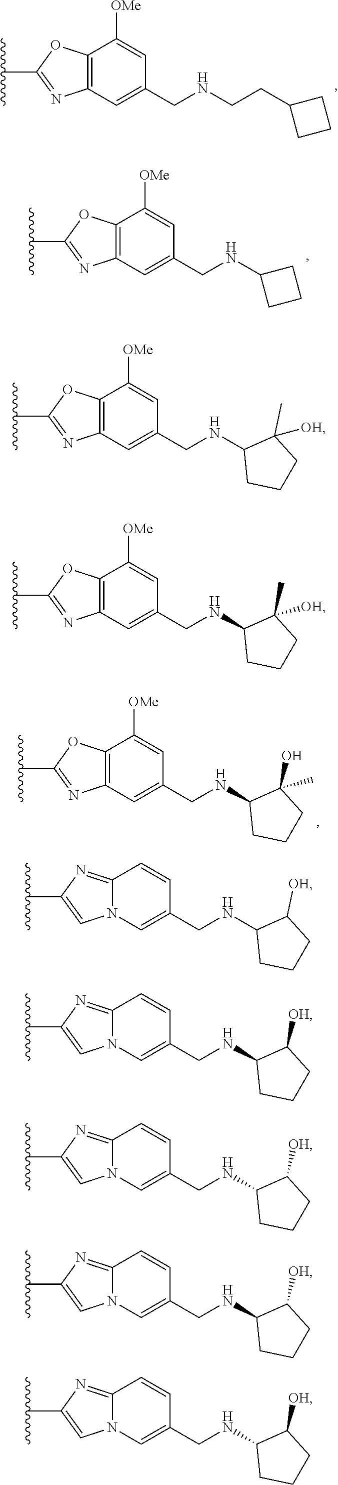

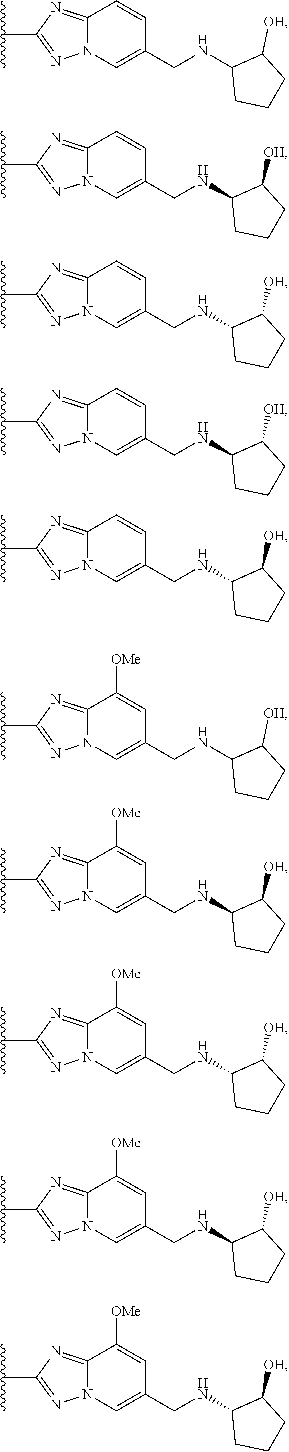

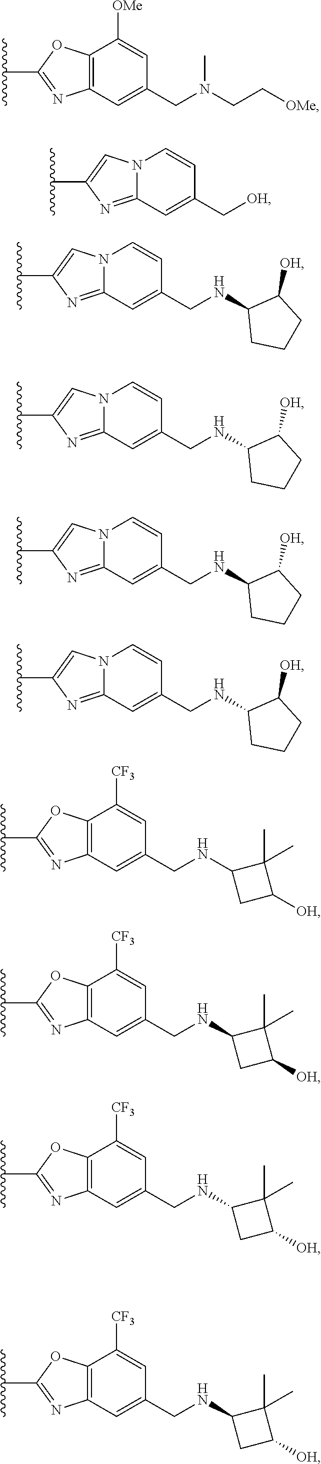

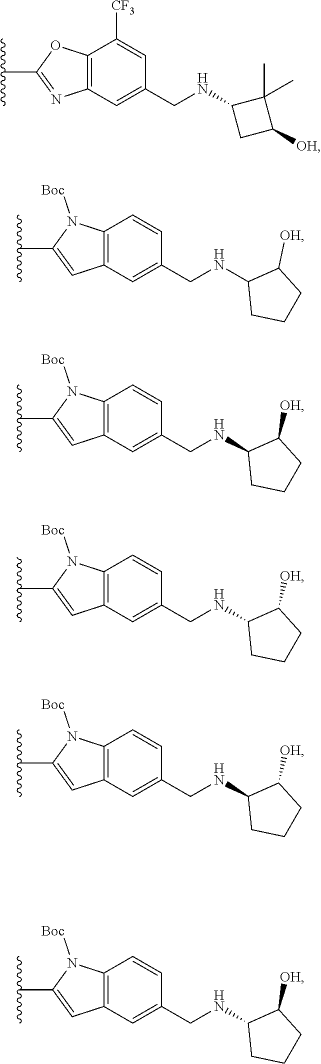 [SMILES: *c1nc2cc(CN[C@H]3C[C@H](O)C3(C)C)cc(C(F)(F)F)c2o1 ; *c1cc2cc(CNC3CCCC3O)ccc2n1C(=O)OC(C)(C)C ; *c1cc2cc(CN[C@@H]3CCC[C@@H]3O)ccc2n1C(=O)OC(C)(C)C ; *c1cc2cc(CN[C@H]3CCC[C@H]3O)ccc2n1C(=O)OC(C)(C)C ; *c1cc2cc(CN[C@@H]3CCC[C@H]3O)ccc2n1C(=O)OC(C)(C)C ; *c1cc2cc(CN[C@H]3CCC[C@@H]3O)ccc2n1C(=O)OC(C)(C)C]

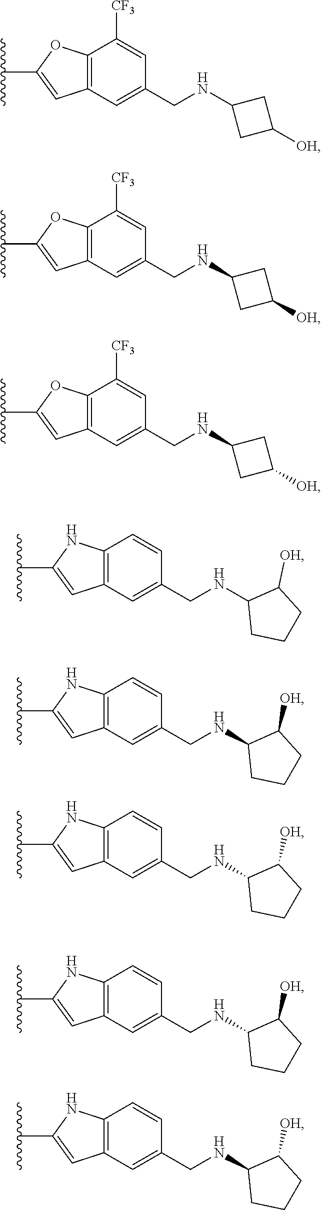

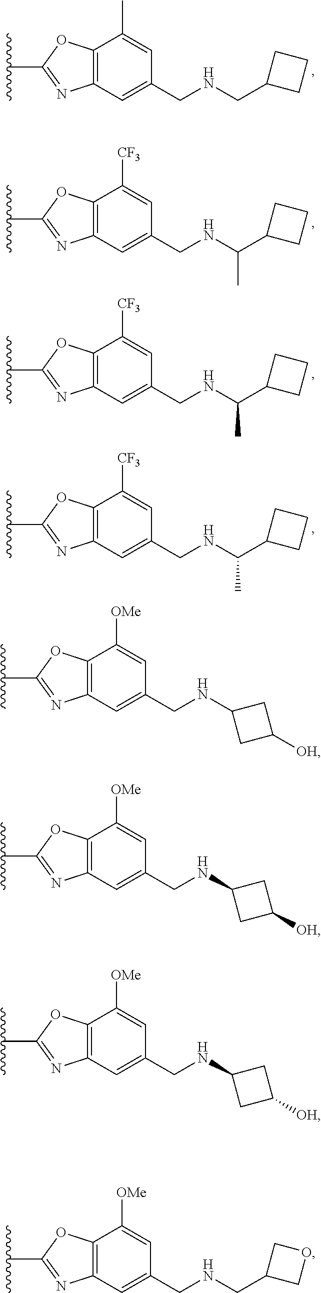

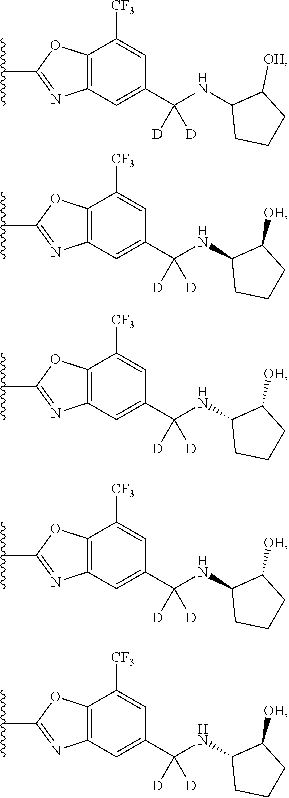 [SMILES: *c1nc2cc(C([2H])([2H])NC3CCCC3O)cc(C(F)(F)F)c2o1 ; *c1nc2cc(C([2H])([2H])N[C@@H]3CCC[C@@H]3O)cc(C(F)(F)F)c2o1 ; *c1nc2cc(C([2H])([2H])N[C@H]3CCC[C@H]3O)cc(C(F)(F)F)c2o1 ; *c1nc2cc(C([2H])([2H])N[C@@H]3CCC[C@H]3O)cc(C(F)(F)F)c2o1 ; *c1nc2cc(C([2H])([2H])N[C@H]3CCC[C@@H]3O)cc(C(F)(F)F)c2o1]

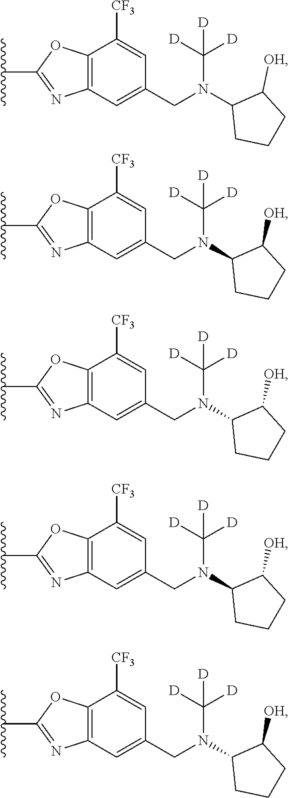 [SMILES: *c1nc2cc(CN(C3CCCC3O)C([2H])([2H])[2H])cc(C(F)(F)F)c2o1 ; *c1nc2cc(CN([C@@H]3CCC[C@@H]3O)C([2H])([2H])[2H])cc(C(F)(F)F)c2o1 ; *c1nc2cc(CN([C@H]3CCC[C@H]3O)C([2H])([2H])[2H])cc(C(F)(F)F)c2o1 ; *c1nc2cc(CN([C@@H]3CCC[C@H]3O)C([2H])([2H])[2H])cc(C(F)(F)F)c2o1 ; *c1nc2cc(CN([C@H]3CCC[C@@H]3O)C([2H])([2H])[2H])cc(C(F)(F)F)c2o1]

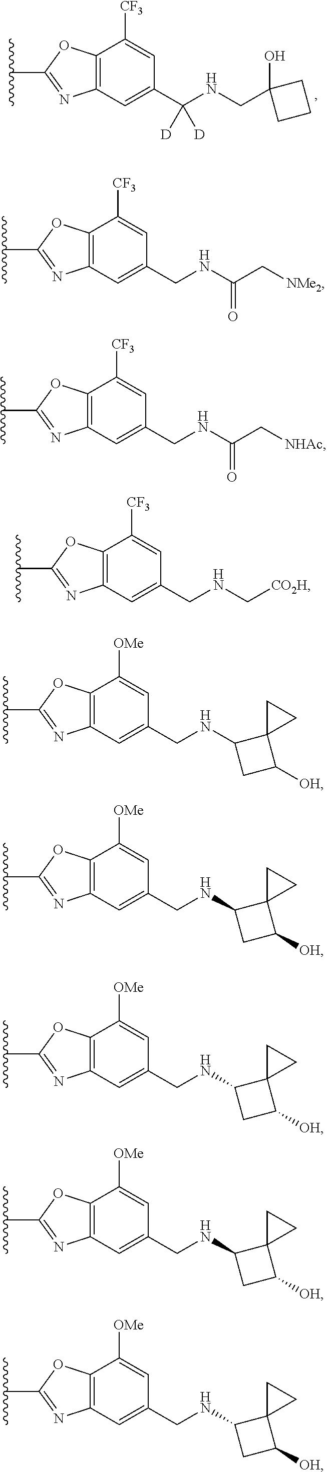

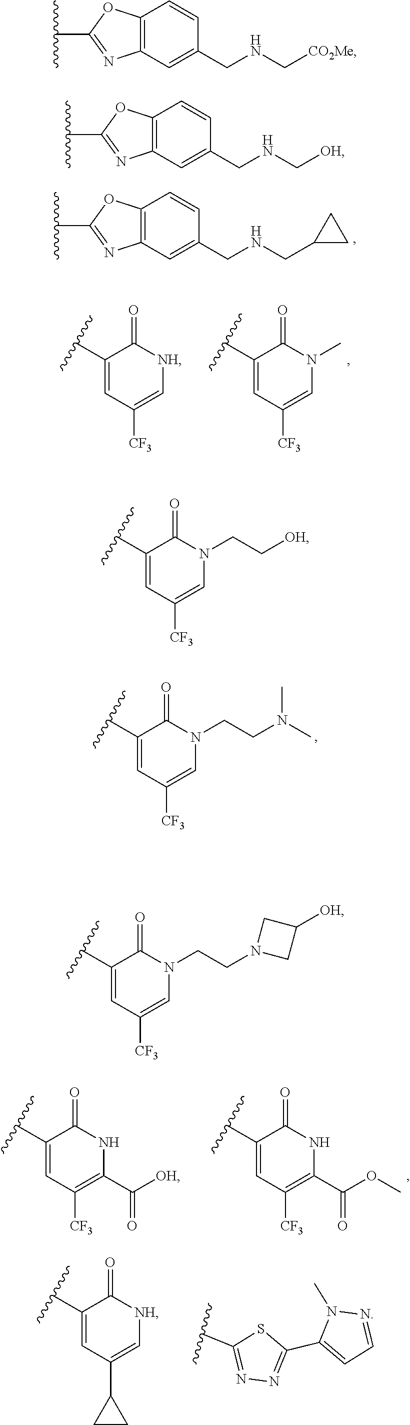

In certain embodiments, each R⁸ is independently hydrogen, oxo, halogen, —CN, —NO₂, —CHF₂, —CH₂F, —CF₃, —OR, —SR, —SF₅, —N(R)₂, —S(O)₂R, —S(O)₂N(R)₂, —S(O)R, —S(O)N(R)₂, —C(O)R, —C(O)OR, —C(O)N(R)₂, —OC(O)R, —OC(O)N(R)₂, —N(R)C(O)OR, —N(R)C(O)R, —N(R)C(O)N(R)₂, —N(R)S(O)₂R, or —N(R)S(O)R; or each R⁸ is independently an optionally substituted groups selected from C₁₋₆ aliphatic and 3-7 membered saturated or partially unsaturated monocyclic carbocyclic ring.

In some embodiments, each R⁸ is independently hydrogen, halogen, —CN, —NO₂, —CHF₂, —CF₃, —OR, —N(R)₂, —C(O)R, —C(O)OR, —C(O)N(R)₂, —OC(O)R, —N(R)C(O)OR, or —N(R)C(O)R; or each R⁸ is independently an optionally substituted C₁₋₆ aliphatic or 3-7 membered saturated or partially unsaturated monocyclic carbocyclic ring.

In some embodiments, each R⁸ is independently hydrogen, halogen, —CF₃, —OR, —N(R)₂, an optionally substituted C₁₋₆ aliphatic or 3-7 membered saturated or partially unsaturated monocyclic carbocyclic ring.

In some embodiments, each R⁸ is C₁₋₆ aliphatic optionally substituted with halogen, —CN, —NO₂, —CHF₂, —CF₃, —OR, —N(R)₂, —C(O)R, —C(O)OR, —C(O)N(R)₂, —OC(O)R, —N(R)C(O)OR, or —N(R)C(O)R.

In some embodiments, each R⁸ is independently -Me, —CF₃, —CF₂H, —CF₂Me, cyclopropyl, -tBu, —Br, —Cl, —F, —OMe, —OCF₃, or —NH₂.

In some embodiments, each R⁸ is independently -Me, —CF₃, or —F.

In some embodiments, each R⁸ is independently hydrogen (i.e., q is 0).

As defined generally above, q is 0, 1, 2, or 3.

In certain embodiments, q is 0, 1, 2, or 3.

In certain embodiments, q is 0. In certain embodiments, q is 1. In certain embodiments, q is 2. In certain embodiments, q is 3.

In certain embodiments, q is 0, 1, or 2. In certain embodiments, q is 1, 2, or 3. In certain embodiments, q is 1 or 2. In certain embodiments, q is 2 or 3.

In some embodiments, q is 1 and R⁸ is -Me, —CF₃, or —F.

In some embodiments, q is 2 and each R⁸ is independently -Me, —CF₃, or —F.

In some embodiments, each R⁸ and q is independently selected from those depicted in Table 1, below.

As defined generally above, L is a covalent bond; or L is a C₁₋₄ bivalent saturated or unsaturated, straight or branched hydrocarbon chain wherein one or two methylene units of the chain are optionally and independently replaced by —C(R)₂—, —N(R)—, —O—, —S—, —S(O)—, —S(O)₂—, —S(O)N(R)—, —N(R)S(O)—, —S(O)₂N(R)—, —N(R)S(O)₂—, —C(O)—, —C(O)O—, —OC(O)—, —C(O)N(R)—, —N(R)C(O)—, —C(O)N(R)O—, —ON(R)C(O)—, —OC(O)N(R)—, —N(R)C(O)O—, or —N(R)C(O)N(R)—.

In certain embodiments, L is a covalent bond. In certain embodiments, L is a C₁₋₄ bivalent saturated or unsaturated, straight or branched hydrocarbon chain wherein one or two methylene units of the chain are optionally and independently replaced by —C(R)₂—, —N(R)—, —O—, —S—, —S(O)—, —S(O)₂—, —S(O)N(R)—, —N(R)S(O)—, —S(O)₂N(R)—, —N(R)S(O)₂—, —C(O)—, —C(O)O—, —OC(O)—, —C(O)N(R)—, —N(R)C(O)—, —C(O)N(R)O—, —ON(R)C(O)—, —OC(O)N(R)—, —N(R)C(O)O—, or —N(R)C(O)N(R)—.

In certain embodiments, L is —CH₂—, —CH₂CH₂—, —CH(Me)-, —CH(OMe)-, —C(Me)₂-, —O—, —OCH₂—, —OCH₂CH₂—, —CH₂NH—, —CD₂NH—, —CH₂N(Me)-, —CH₂N(CD₃)-, —CH₂N(Et)-, —CH₂N(CH₂CH₂OMe)-, —CH₂NHCH₂—, —CH₂N(Me)CH₂—, —CH(Me)NHCH₂—, —CH(Me)N(Me)CH₂—, —C(Me)₂NHCH₂—, —C(Me)₂N(Me)CH₂—, —CH₂NHCH(Me)-, —CH₂N(Me)CH(Me)-, —CH₂NHC(Me)₂-, —CH₂NHCH₂CH₂—, —CH₂N(Me)CH₂CH₂—, —CH₂N(Et)CH₂CH₂—, —CH₂NHCH₂CH(Me)-, —CH₂NHCH₂C(Me)₂-, —CH₂N(Me)CH₂C(Me)₂-, —C(O)—, —C(O)NH—, —NHC(O)—,

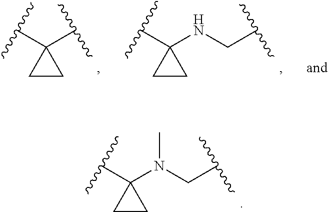

In certain embodiments, L is —CH₂NR—, —CH₂NRCH₂—, —CH₂NRCH₂CH₂—, or —CH₂NRCH₂C(Me)₂-.

In certain embodiments, L is —CH₂NH—, —CH₂NHCH₂—, —CH₂NHCH₂CH₂—, or —CH₂NHCH₂C(Me)₂-.

In some embodiments, L is selected from those depicted in Table 1, below.

As defined generally above, R⁹ is halogen, —CN, —NO₂, —OR, —SR, —N(R)₂, —S(O)₂R, —S(O)₂N(R)₂, —S(O)R, —S(O)N(R)₂, —CF₂R, —CF₃, —C(R)₂OR, —C(R)₂N(R)₂, —C(O)R, —C(O)OR, —C(O)N(R)₂, —C(O)N(R)OR, —OC(O)R, —OC(O)N(R)₂, —C(S)N(R)₂, —N(R)C(O)OR, —N(R)C(O)R, —N(R)C(O)N(R)₂, —N(R)C(NR)N(R)₂, —N(R)N(R)₂, —N(R)S(O)₂N(R)₂, —N(R)S(O)₂R, —N═S(O)(R)₂, —S(NR)(O)R, —N(R)S(O)R, —N(R)CN, —Si(OR)(R)₂, —Si(R)₃, —P(O)(R)N(R)₂, —P(O)(R)OR, or —P(O)(R)₂; or R⁹ is an optionally substituted group selected from C₁₋₆ aliphatic; phenyl; a 3-7 membered saturated or partially unsaturated monocyclic carbocyclic ring; a 4-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; a 5-8 membered saturated or partially unsaturated bridged bicyclic ring having 0-3 heteroatoms selected from nitrogen, oxygen, and sulfur; a 6-10 membered saturated or partially unsaturated spiro bicyclic ring having 0-3 heteroatoms independently selected from nitrogen, oxygen, and sulfur; and an 8-10 membered partially aromatic or heteroaromatic bicyclic heterocyclic ring having 1-4 heteroatoms independently selected from nitrogen, oxygen, and sulfur.

In certain embodiments, R⁹ is halogen, —CN, —NO₂, —OR, —SR, —N(R)₂, —S(O)₂R, —S(O)₂N(R)₂, —S(O)R, —S(O)N(R)₂, —CF₂R, —CF₃, —C(R)₂OR, —C(R)₂N(R)₂, —C(O)R, —C(O)OR, —C(O)N(R)₂, —C(O)N(R)OR, —OC(O)R, —OC(O)N(R)₂, —C(S)N(R)₂, —N(R)C(O)OR, —N(R)C(O)R, —N(R)C(O)N(R)₂, —N(R)C(NR)N(R)₂, —N(R)N(R)₂, —N(R)S(O)₂N(R)₂, —N(R)S(O)₂R, —N═S(O)(R)₂, —S(NR)(O)R, —N(R)S(O)R, —N(R)CN, —Si(OR)(R)₂, —Si(R)₃, —P(O)(R)N(R)₂, —P(O)(R)OR, or —P(O)(R)₂; or R⁹ is an optionally substituted group selected from C₁₋₆ aliphatic; phenyl; a 3-7 membered saturated or partially unsaturated monocyclic carbocyclic ring; a 4-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; a 5-8 membered saturated or partially unsaturated bridged bicyclic ring having 0-3 heteroatoms selected from nitrogen, oxygen, and sulfur; a 6-10 membered saturated or partially unsaturated spiro bicyclic ring having 0-3 heteroatoms independently selected from nitrogen, oxygen, and sulfur; and an 8-10 membered partially aromatic or heteroaromatic bicyclic heterocyclic ring having 1-4 heteroatoms independently selected from nitrogen, oxygen, and sulfur.

In certain embodiments, R⁹ is -Me, -Et, —Pr, -i-Pr, straight chain or branched butyl, straight chain or branched pentyl, straight chain or branched hexyl, halogen, —CN, —NO₂, —OR, —SR, —N(R)₂, —S(O)₂R, —S(O)₂N(R)₂, —S(O)R, —S(O)N(R)₂, —CF₂R, —CF₃, —C(R)₂OR, —C(R)₂N(R)₂, —C(O)R, —C(O)OR, —C(O)N(R)₂, —C(O)N(R)OR, —OC(O)R, —OC(O)N(R)₂, —C(S)N(R)₂, —N(R)C(O)OR, —N(R)C(O)R, —N(R)C(O)N(R)₂, —N(R)C(NR)N(R)₂, —N(R)N(R)₂, —N(R)S(O)₂N(R)₂, —N(R)S(O)₂R, —N═S(O)(R)₂, —S(NR)(O)R, —N(R)S(O)R, —N(R)CN, —Si(OR)(R)₂, —Si(R)₃, —P(O)(R)N(R)₂, —P(O)(R)OR, or —P(O)(R)₂; or R⁹ is an optionally substituted cyclopropyl, cyclobutyl, cyclopentyl, cyclohexyl, oxetanyl, azetidinyl, furanyl, furazanyl, imidazolyl, imidazolidinyl, imidazolinyl, isothiazolyl, isoxazolyl, oxadiazolyl, oxazolidinyl, oxazolyl, oxazoldinyl, morpholinyl, piperidinyl, piperazinyl, pyrazolidinyl, pyrrolidinyl, pyranyl, pyrazinyl, pyrazolinyl, pyrazolyl, pyridazinyl, pyridinyl, pyrimidinyl, pyrrolinyl, pyrrolyl, thiadiazolyl, thienyl, triazinyl, or triazolyl.

In certain embodiments, R⁹ is -Me, -Et, —Pr, -i-Pr, straight chain or branched butyl, straight chain or branched pentyl, straight chain or branched hexyl, halogen, —CN, —OR, —N(R)₂, —CF₃, —C(O)R, —C(O)OR, —C(O)N(R)₂, —OC(O)R, —OC(O)N(R)₂, —N(R)C(O)OR, —N(R)C(O)R, —N(R)C(O)N(R)₂, —N(R)S(O)₂R, or —N(R)S(O)R; or R⁹ is an optionally substituted cyclopropyl, cyclobutyl, cyclopentyl, cyclohexyl, oxetanyl, azetidinyl, imidazolyl, imidazolidinyl, morpholinyl, piperidinyl, piperazinyl, pyrazolidinyl, pyrrolidinyl, pyridinyl, or oxazolidinyl.

In certain embodiments, R⁹ is -Me, -Et, —Pr, -i-Pr, straight chain or branched butyl, straight chain or branched pentyl, straight chain or branched hexyl, —OR, —N(R)₂; or R⁹ is an optionally substituted group selected from cyclopropyl, cyclobutyl, cyclopentyl, cyclohexyl, oxetanyl, azetidinyl, pyrrolidinyl, piperidinyl, piperazinyl, morpholinyl, pyridinyl, and imidazolyl.

In certain embodiments, R⁹ is —OR,

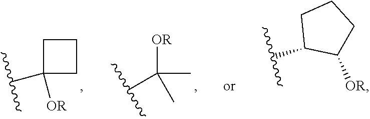

wherein R is hydrogen or C₁₋₆alkyl (e.g., methyl, ethyl, propyl or isopropyl).

In certain embodiments, R⁹ is —F, -Me, —CF₂H, —CO₂H, —NH₂, —NHMe, —NHEt, —NHAc, —NMe₂, —OH, —OMe, —OEt, —OiPr, —SO₂Me,

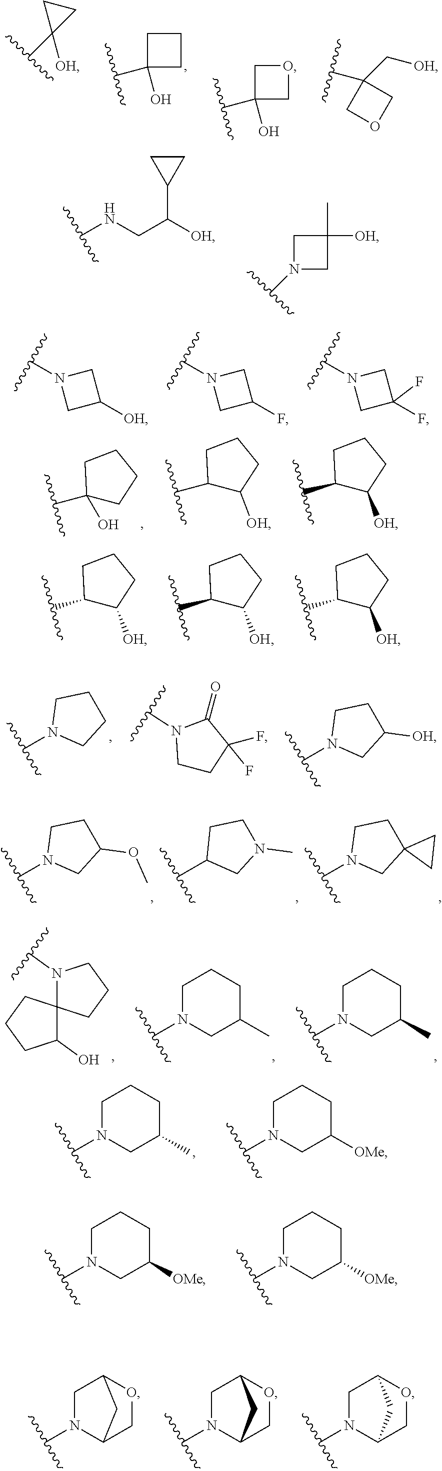

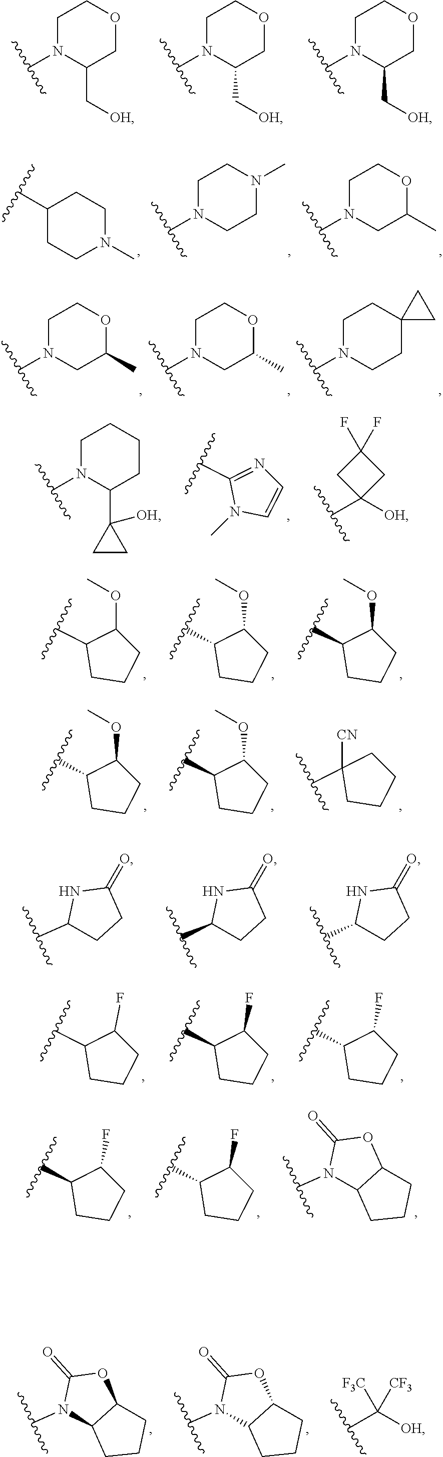

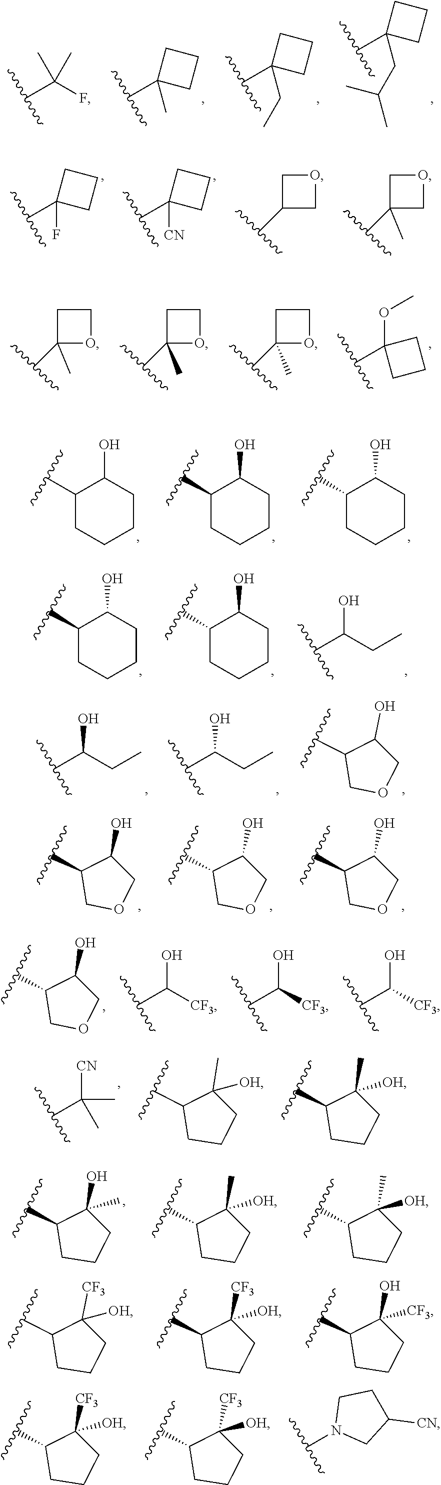

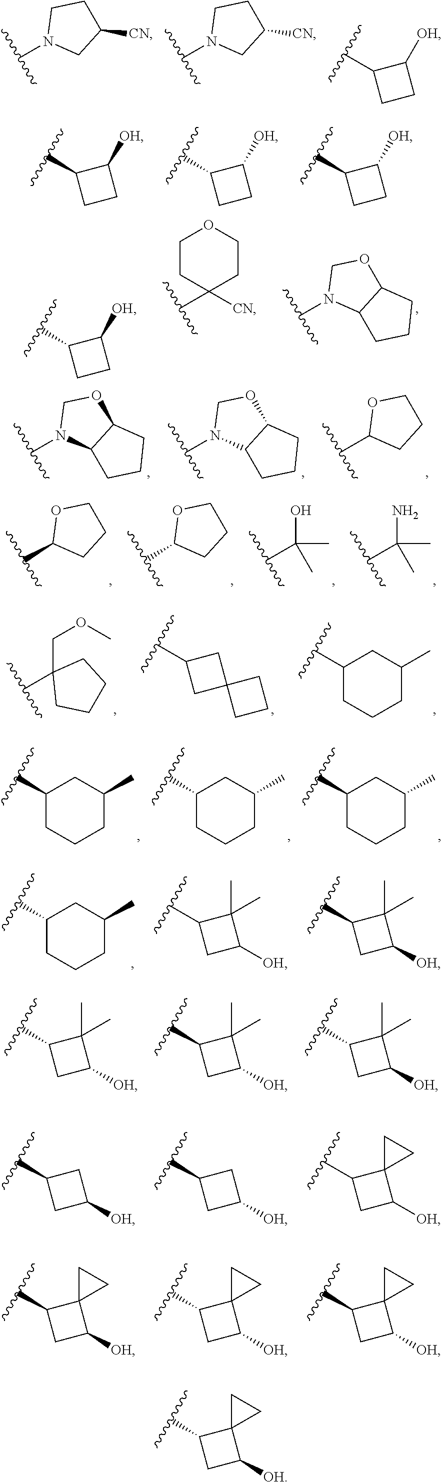

In certain embodiments, R⁹ is —OH, OMe,

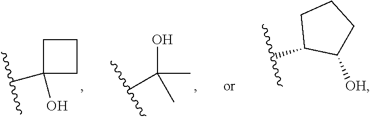

In some embodiments, R⁹ is selected from those depicted in Table 1, below.

In certain embodiments, -L-R⁹ is

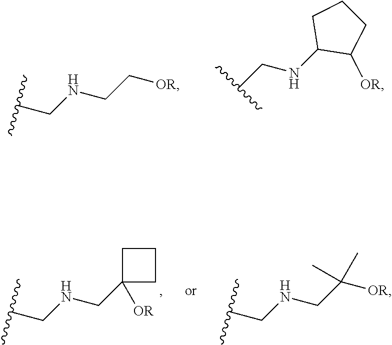

wherein R is hydrogen or C₁₋₆alkyl (e.g., methyl, ethyl, propyl or isopropyl).

In certain embodiments, -L-R⁹ is

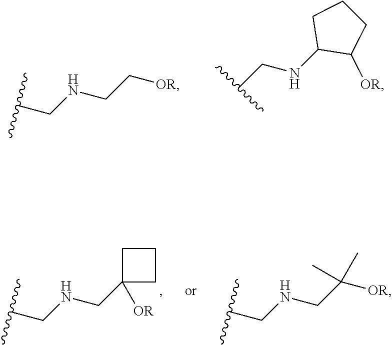

wherein R is hydrogen or methyl.

In some embodiments, -L-R⁹ is

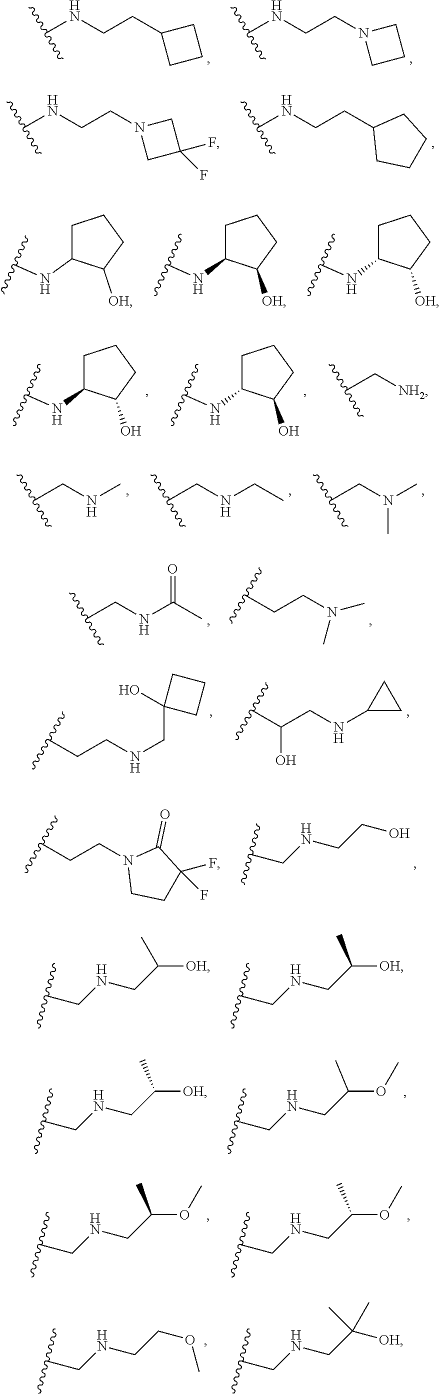

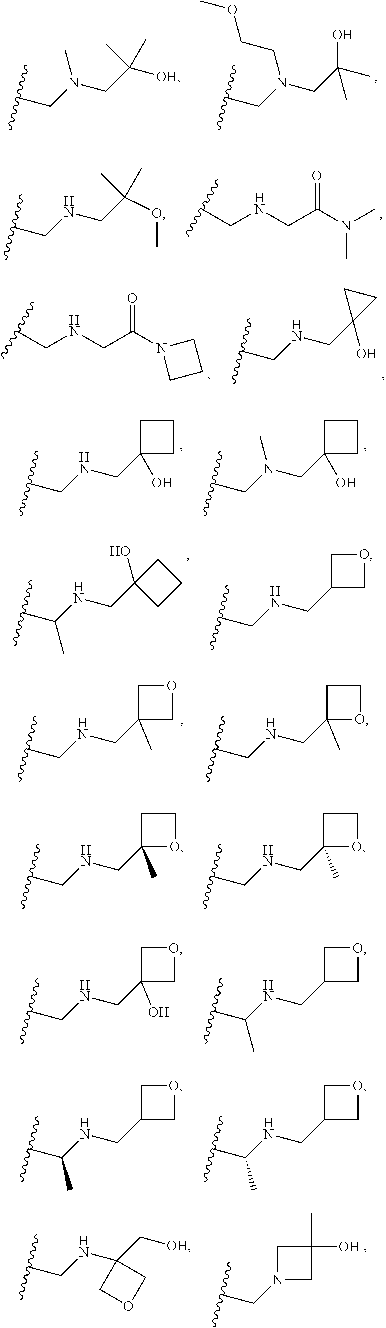

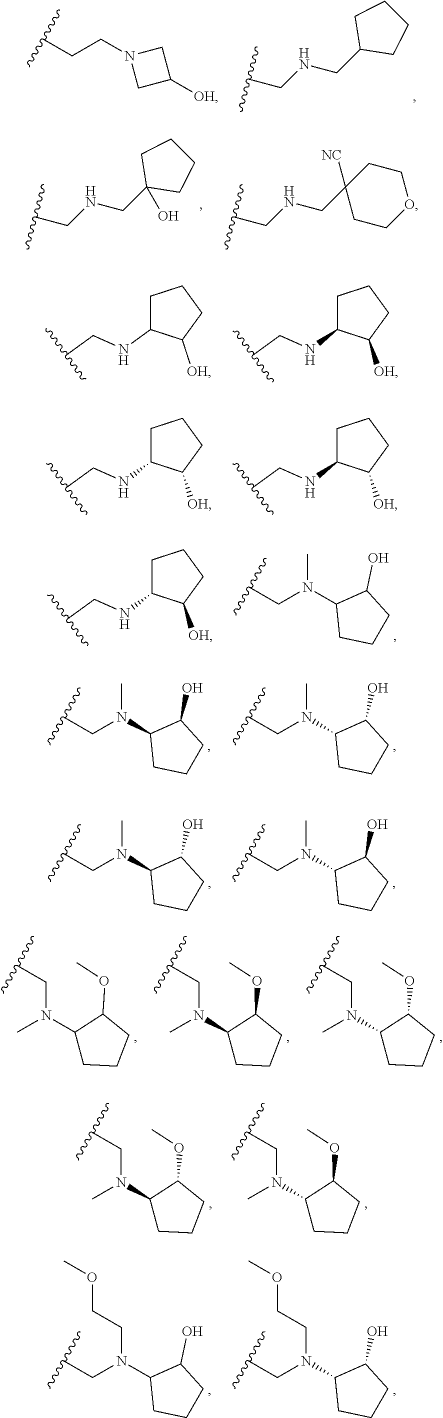

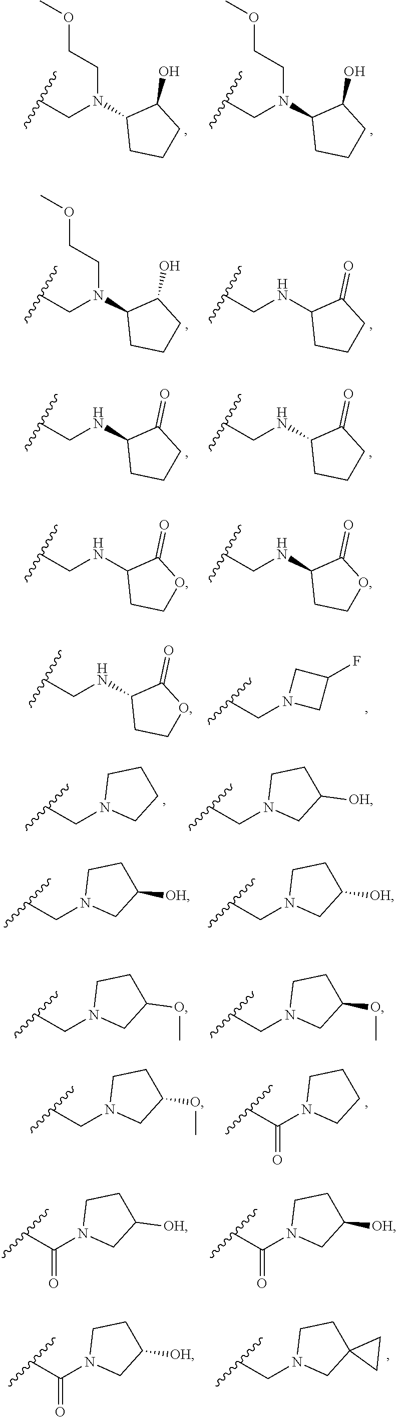

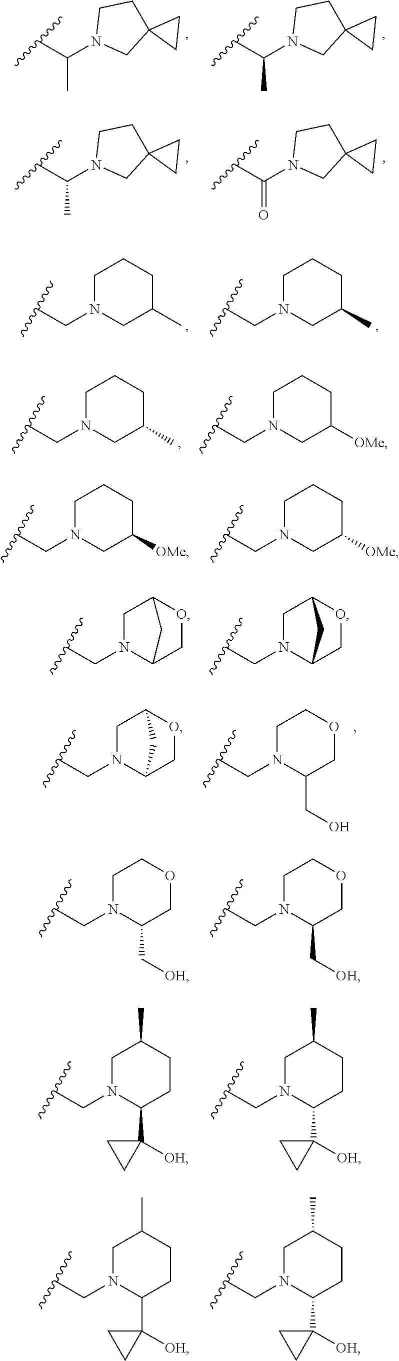

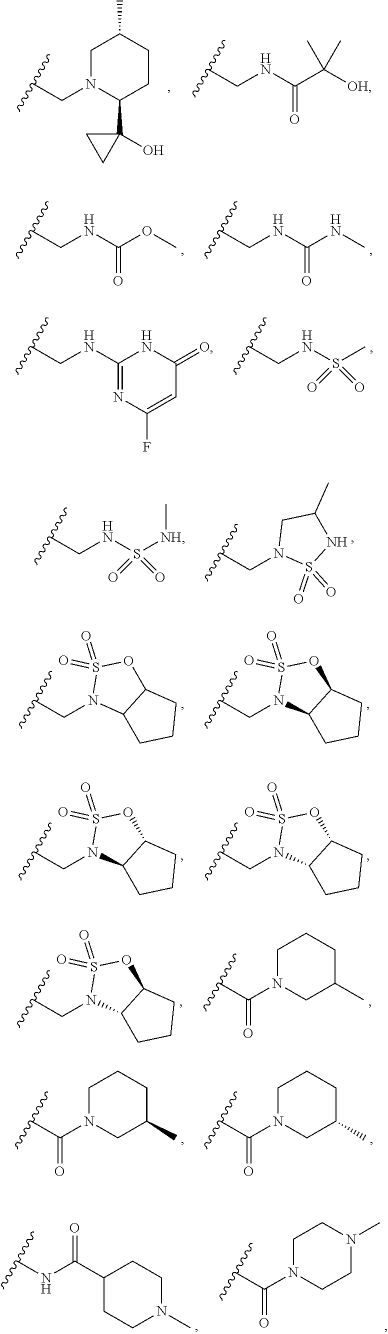

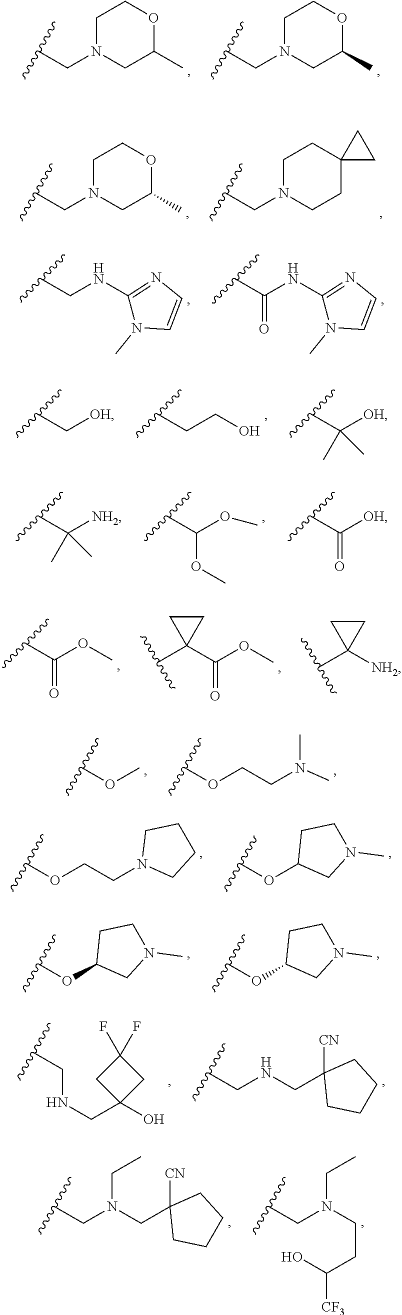

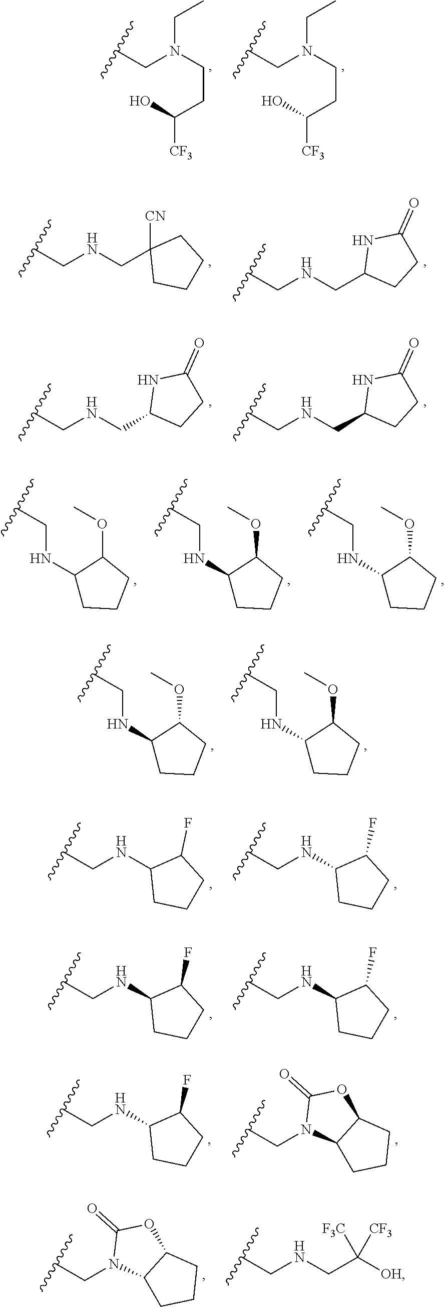

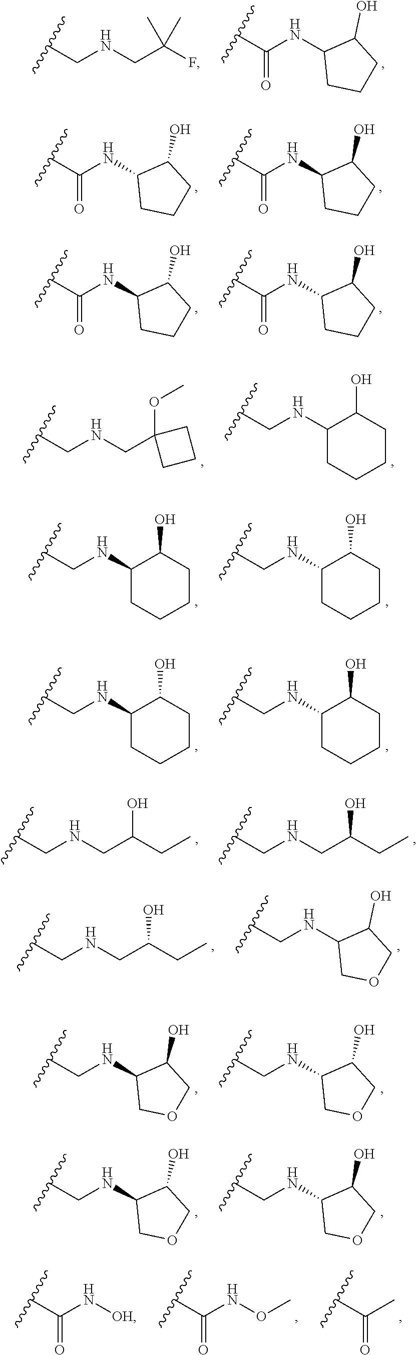

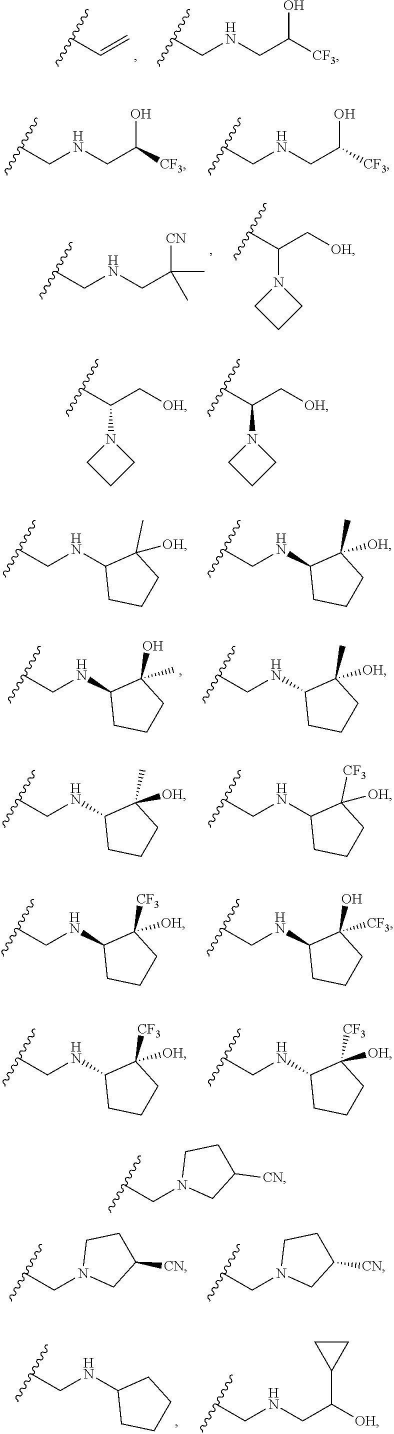

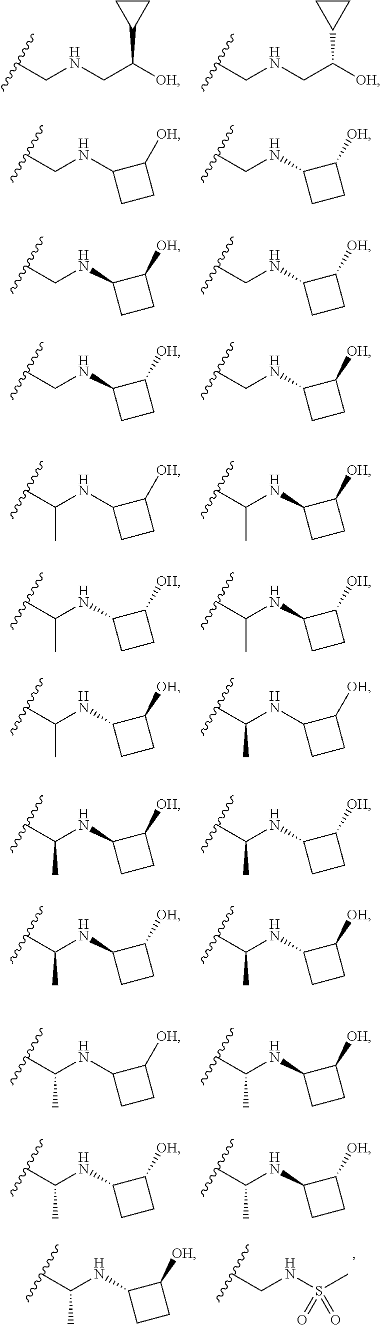

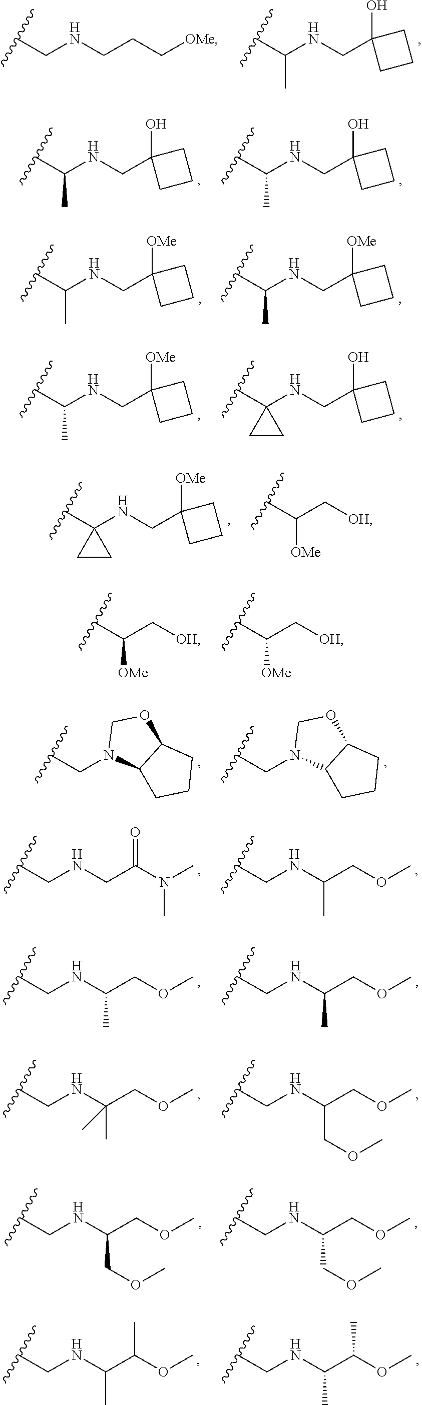

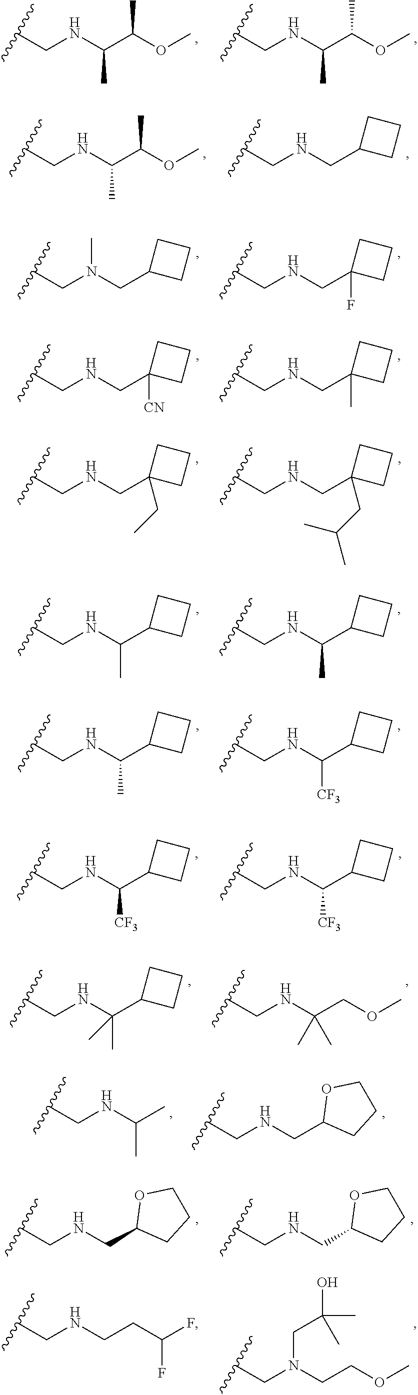

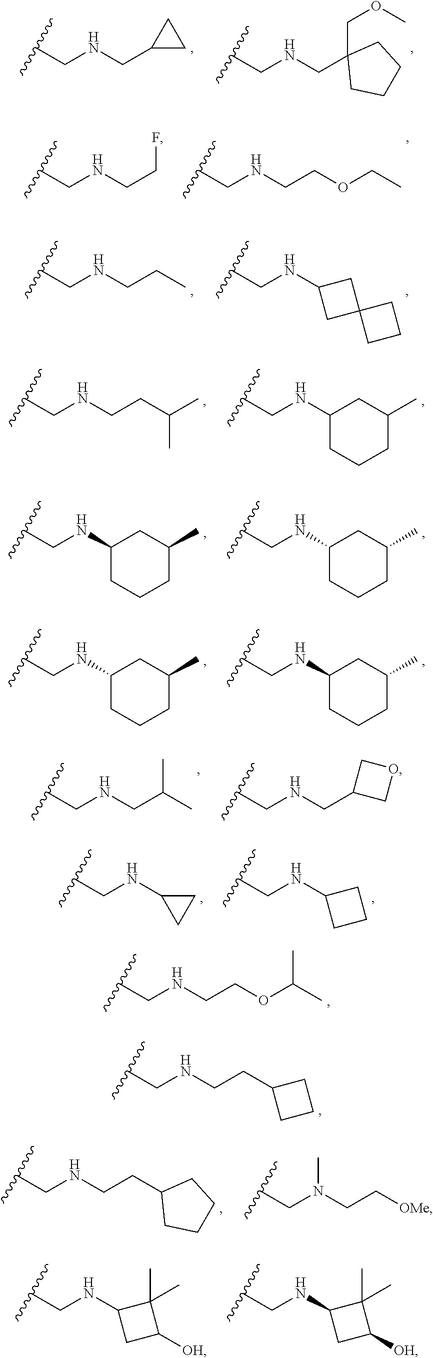

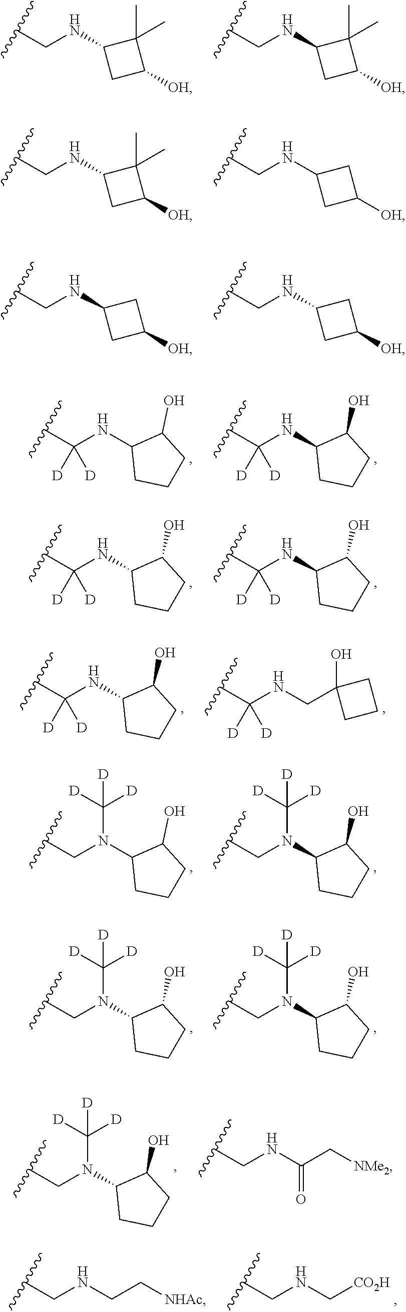

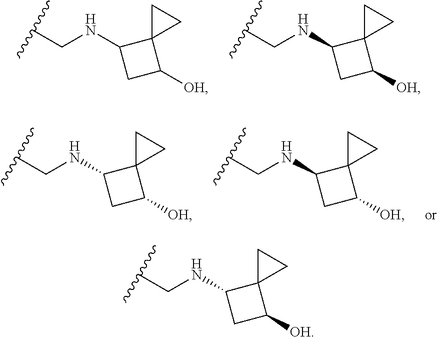 [SMILES: *CNC1CC(O)C12CC2 ; *CN[C@@H]1C[C@H](O)C12CC2 ; *CN[C@H]1C[C@@H](O)C12CC2 ; *CN[C@@H]1C[C@@H](O)C12CC2 ; *CN[C@H]1C[C@H](O)C12CC2]

In certain embodiments, -L-R⁹ is

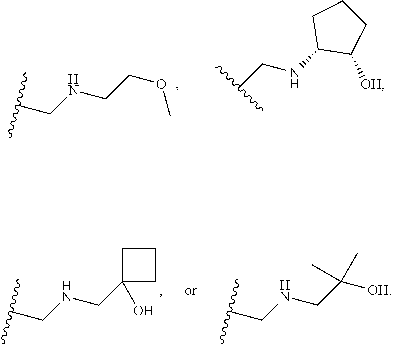

As defined generally above, t is 0 or 1.

In certain embodiments, t is 0 or 1.

In certain embodiments, t is 0. In certain embodiments, t is 1.

In certain embodiments, t is 1 and -L-R⁹ is

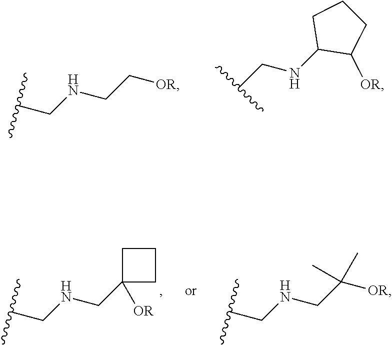

wherein R is hydrogen or C₁₋₆alkyl (e.g., methyl, ethyl, propyl or isopropyl).

In certain embodiments, t is 1 and -L-R⁹ is

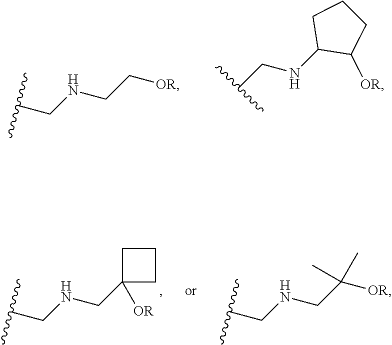

wherein R is hydrogen or methyl.

In certain embodiments, t is 1 and -L-R⁹ is

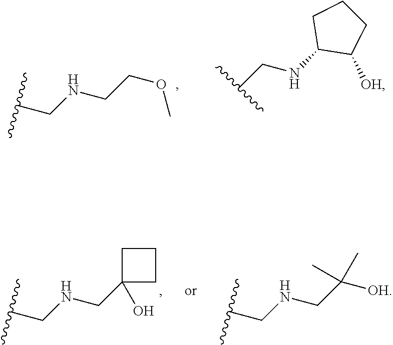

In some embodiments, each -L-R⁹ and t is selected from those depicted in Table 1, below.

As defined generally above, each instance of R¹, R², R³, R⁴, R⁵, R⁶, R⁷, R⁸, and R⁹ is independently and optionally substituted with v instances of R^{A}, wherein each R^{A} is independently oxo, halogen, —CN, —NO₂, —OR, —SR, —N(R)₂, —S(O)₂R, —S(O)₂N(R)₂, —S(O)R, —S(O)N(R)₂, —C(R)₂OR, —C(O)R, —C(O)OR, —C(O)N(R)₂, —C(O)N(R)OR, —OC(O)R, —OC(O)N(R)₂, —N(R)C(O)OR, —N(R)C(O)R, —N(R)C(O)N(R)₂, —N(R)C(NR)N(R)₂, —N(R)N(R)₂, —N(R)S(O)₂N(R)₂, —N(R)S(O)₂R, —N═S(O)(R)₂, —S(NR)(O)R, —N(R)S(O)R, —N(R)CN, —P(O)(R)N(R)₂, —P(O)(R)OR, —P(O)(R)₂, or an optionally substituted group selected from C₁₋₆ aliphatic; phenyl; naphthyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, phosphorous, silicon and sulfur; a 5-6 membered monocyclic heteroaryl ring having 1-4 heteroatoms independently selected from nitrogen, oxygen, and sulfur; an 8-10 membered bicyclic heteroaryl ring having 1-5 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; and a 6-11 membered saturated or partially unsaturated bicyclic heterocyclic ring having 1-2 heteroatoms independently selected from nitrogen, oxygen, and sulfur.

In certain embodiments, R¹, R², R³, R⁴, R⁵, R⁶, R⁷, R⁸, or R⁹ is independently and optionally substituted with v instances of R^{A}, wherein each R^{A} is independently oxo, halogen, —CN, —NO₂, —OR, —SR, —N(R)₂, —S(O)₂R, —S(O)₂N(R)₂, —S(O)R, —S(O)N(R)₂, —C(R)₂OR, —C(O)R, —C(O)OR, —C(O)N(R)₂, —C(O)N(R)OR, —OC(O)R, —OC(O)N(R)₂, —N(R)C(O)OR, —N(R)C(O)R, —N(R)C(O)N(R)₂, —N(R)C(NR)N(R)₂, —N(R)N(R)₂, —N(R)S(O)₂N(R)₂, —N(R)S(O)₂R, —N═S(O)(R)₂, —S(NR)(O)R, —N(R)S(O)R, —N(R)CN, —P(O)(R)N(R)₂, —P(O)(R)OR, —P(O)(R)₂, or an optionally substituted group selected from C₁₋₆ aliphatic; phenyl; naphthyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, phosphorous, silicon and sulfur; a 5-6 membered monocyclic heteroaryl ring having 1-4 heteroatoms independently selected from nitrogen, oxygen, and sulfur; an 8-10 membered bicyclic heteroaryl ring having 1-5 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; and a 6-11 membered saturated or partially unsaturated bicyclic heterocyclic ring having 1-2 heteroatoms independently selected from nitrogen, oxygen, and sulfur.

In some embodiments, R^{A} is oxo, halogen, —CN, —NO₂, —OR, —SR, —N(R)₂, —S(O)₂P, —S(O)₂N(R)₂, —S(O)R, —S(O)N(R)₂, —C(R)₂OR, —C(O)R, —C(O)OR, —C(O)N(R)₂, —C(O)N(R)OR, —OC(O)R, —OC(O)N(R)₂, —N(R)C(O)OR, —N(R)C(O)R, —N(R)C(O)N(R)₂, —N(R)C(NR)N(R)₂, —N(R)N(R)₂, —N(R)S(O)₂N(R)₂, —N(R)S(O)₂R, —N═S(O)(R)₂, —S(NR)(O)R, —N(R)S(O)R, —N(R)CN, —P(O)(R)N(R)₂, —P(O)(R)OR, —P(O)(R)₂, or an optionally substituted group selected from C₁₋₆ aliphatic; phenyl; naphthyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, phosphorous, silicon and sulfur; a 5-6 membered monocyclic heteroaryl ring having 1-4 heteroatoms independently selected from nitrogen, oxygen, and sulfur; an 8-10 membered bicyclic heteroaryl ring having 1-5 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; and a 6-11 membered saturated or partially unsaturated bicyclic heterocyclic ring having 1-2 heteroatoms independently selected from nitrogen, oxygen, and sulfur.

In some embodiments, R^{A} is methyl, —CF₃, —OH, —OMe, fluoro, chloro, —CO₂Me, or —NH₂.

In some embodiments, R^{A} is selected from those depicted in Table 1, below.

As defined generally above, each R is independently hydrogen, —CN, halogen, or an optionally substituted group selected from C₁₋₆ aliphatic; phenyl; naphthyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; a 6-11 membered saturated or partially unsaturated bicyclic carbocyclic ring having 1-2 heteroatoms independently selected from nitrogen, oxygen, and sulfur; or two R groups on the same atom are optionally taken together with the atom to form an optionally substituted 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur.

In some embodiments, R is hydrogen, —CN, halogen, or an optionally substituted group selected from C₁₋₆ aliphatic; phenyl; naphthyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; a 6-11 membered saturated or partially unsaturated bicyclic carbocyclic ring having 1-2 heteroatoms independently selected from nitrogen, oxygen, and sulfur; or two R groups on the same atom are optionally taken together with the atom to form an optionally substituted 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur.

In some embodiments, R is hydrogen.

In some embodiments, R is —CN or halogen.

In some embodiments, R is an optionally substituted group selected from C₁₋₆ aliphatic; phenyl; naphthyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; a 6-11 membered saturated or partially unsaturated bicyclic carbocyclic ring having 1-2 heteroatoms independently selected from nitrogen, oxygen, and sulfur.

In some embodiments, two R groups on the same atom are taken together with the atom to form an optionally substituted 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur.

In some embodiments, R is selected from those depicted in Table 1, below.

As defined generally above, each instance of v is independently 0, 1, 2, 3, 4, or 5.

In some embodiments, v is 0, 1, 2, 3, 4, or 5.

In some embodiments, v is 0. In some embodiments, v is 1. In some embodiments, v is 2. In some embodiments, v is 3. In some embodiments, v is 4. In some embodiments, v is 5.

In some embodiments, v is 0 or 1. In some embodiments, v is 0, 1, or 2. In some embodiments, v is 0, 1, 2, or 3. In some embodiments, v is 1 or 2. In some embodiments, v is 1, 2, or 3. In some embodiments, v is 1, 2, 3, or 4. In some embodiments, v is 2 or 3. In some embodiments, v is 2, 3, or 4. In some embodiments, v is 3 or 4. In some embodiments, v is 3, 4, or 5.

In some embodiments, v is selected from those depicted in Table 1, below.

In certain embodiments, w is 0.

[symbol]

In certain embodiments, w is 1.

As defined generally above, [symbol] denotes a single or double bond.

In some embodiments, [symbol] denotes a single bond. In some embodiments, [symbol] denotes a double bond.

In some embodiments, [symbol] is selected from those depicted in Table 1, below.

In some embodiments, the present invention provides compounds of formula II-a to II-j:

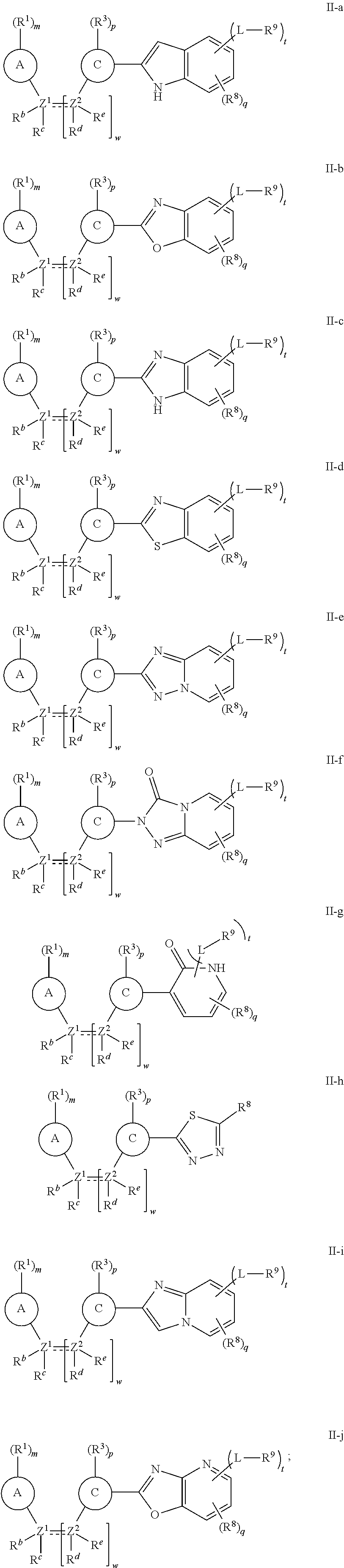

or a pharmaceutically acceptable salt thereof, wherein each of Z¹, Z², Ring A, Ring C, R^{b}, R^{c}, R^{d}, R^{e}, R¹, R³, R⁸, L, R⁹, m, p, q, t, and w, is as defined above and described in embodiments herein, both singly and in combination.

In some embodiments, the present invention provides compounds of formula II-a to II-j, wherein the group

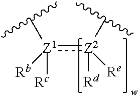

is

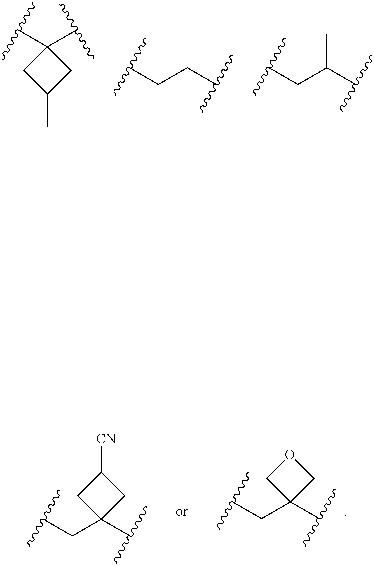

or wherein R^{b} and R^{d} together with the atoms to which each are attached, and Z¹ and Z², forms

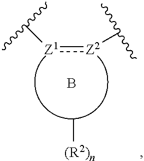

wherein Ring B is a divalent group selected from

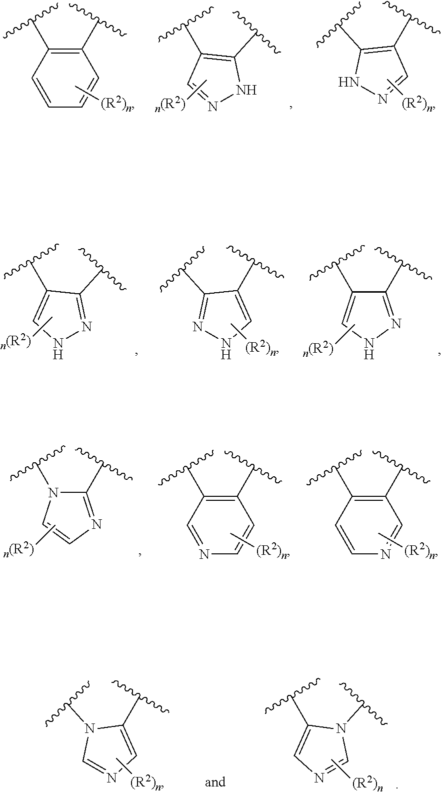

In some embodiments, the present invention provides compounds of formula II-b-1:

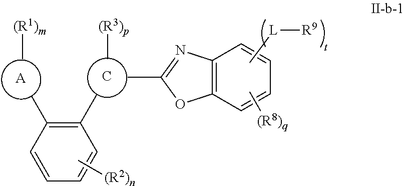

or a pharmaceutically acceptable salt thereof, wherein each of Ring A, Ring C, R¹, R³, R⁸, L, R⁹, m, p, q, t, and w, is as defined above and described in embodiments herein, both singly and in combination.

In some embodiments, the present invention provides compounds of formula II-b-1 wherein Ring C is phenylenyl or pridinylenyl with meta-linkage.

In some embodiments, the present invention provides compounds of formula II-b-2:

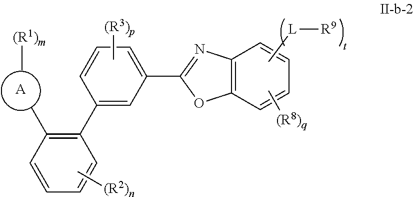

or a pharmaceutically acceptable salt thereof, wherein each of Ring A, R¹, R³, R⁸, L, R⁹, m, p, q, t, and w, is as defined above and described in embodiments herein, both singly and in combination.

In some embodiments, the present invention provides compounds of formula II-b-3:

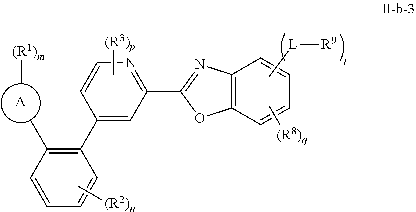

or a pharmaceutically acceptable salt thereof, wherein each of Ring A, R¹, R³, R⁸, L, R⁹, m, p, q, t, and w, is as defined above and described in embodiments herein, both singly and in combination.

In some embodiments, the present invention provides compounds of formula II-b-4:

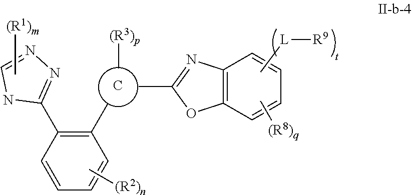

or a pharmaceutically acceptable salt thereof, wherein each of Ring C, R¹, R³, R⁸, L, R⁹, m, p, q, t, and w, is as defined above and described in embodiments herein, both singly and in combination.

In some embodiments, the present invention provides compounds of formula II-b-4 wherein Ring C is phenylenyl or pridinylenyl with meta-linkage.

In some embodiments, the present invention provides compounds of formula II-b-5:

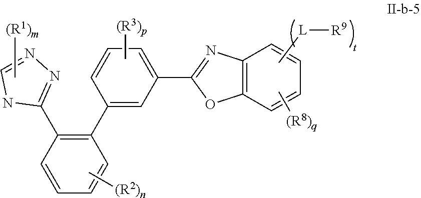

or a pharmaceutically acceptable salt thereof, wherein each of R¹, R³, R⁸, L, R⁹, m, p, t, q, and w, is as defined above and described in embodiments herein, both singly and in combination.

In some embodiments, the present invention provides compounds of formula II-b-6:

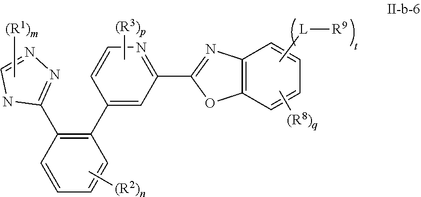

or a pharmaceutically acceptable salt thereof, wherein each of R¹, R³, R⁸, L, R⁹, m, p, t, q, and w, is as defined above and described in embodiments herein, both singly and in combination.

In some embodiments, the present invention provides compounds of formula II-b-7:

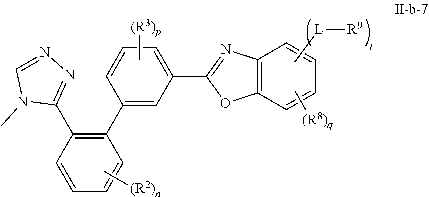

or a pharmaceutically acceptable salt thereof, wherein each of R³, R⁸, L, R⁹, p, t, q, and w, is as defined above and described in embodiments herein, both singly and in combination.

In some embodiments, the present invention provides compounds of formula II-b-8:

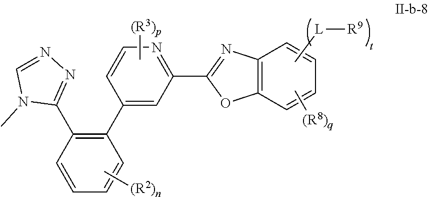

or a pharmaceutically acceptable salt thereof, wherein each of R³, R⁸, L, R⁹, p, t, q, and w, is as defined above and described in embodiments herein, both singly and in combination.

In some embodiments, the present invention provides compounds of formula II-b-9:

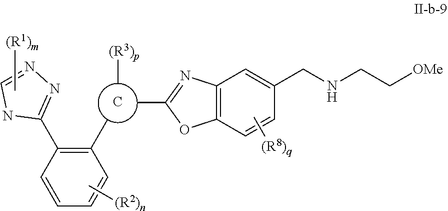

or a pharmaceutically acceptable salt thereof, wherein each of Ring C, R¹, R³, R⁸, m, p, q, and w, is as defined above and described in embodiments herein, both singly and in combination.

In some embodiments, the present invention provides compounds of formula II-b-9 wherein Ring C is phenylenyl or pridinylenyl with meta-linkage.

In some embodiments, the present invention provides compounds of formula II-b-10:

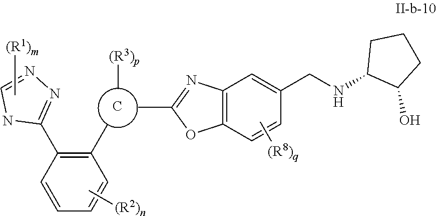

or a pharmaceutically acceptable salt thereof, wherein each of Ring C, R¹, R³, R⁸, m, p, q, and w, is as defined above and described in embodiments herein, both singly and in combination.

In some embodiments, the present invention provides compounds of formula II-b-10 wherein Ring C is phenylenyl or pridinylenyl with meta-linkage.

In some embodiments, the present invention provides compounds of formula II-b-11:

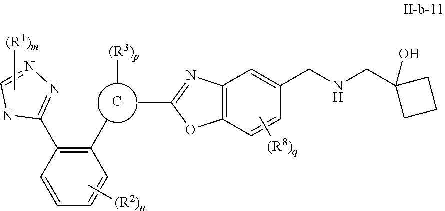

or a pharmaceutically acceptable salt thereof, wherein each of Ring C, R¹, R³, R⁸, m, p, q, and w, is as defined above and described in embodiments herein, both singly and in combination.

In some embodiments, the present invention provides compounds of formula II-b-11 wherein Ring C is phenylenyl or pridinylenyl with meta-linkage.

In some embodiments, the present invention provides compounds of formula II-b-12:

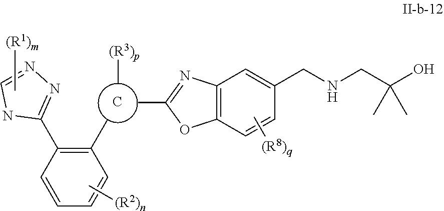

or a pharmaceutically acceptable salt thereof, wherein each of Ring C, R¹, R³, R⁸, m, p, q, and w, is as defined above and described in embodiments herein, both singly and in combination.

In some embodiments, the present invention provides compounds of formula II-b-12 wherein Ring C is phenylenyl or pridinylenyl with meta-linkage.

Exemplary compounds of the invention are set forth in Table 1, below.

TABLE 1
Selected Compounds

| Compound # | Compound ID | Structure |
|---|---|---|
| 1 | F-5 | 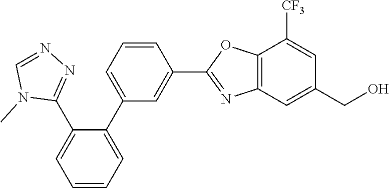 [SMILES: Cn1cnnc1-c1ccccc1-c1cccc(-c2nc3cc(CO)cc(C(F)(F)F)c3o2)c1] |
| 2 | G-1 | 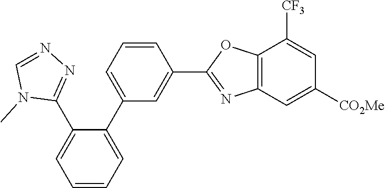 [SMILES: COC(=O)c1cc(C(F)(F)F)c2oc(-c3cccc(-c4ccccc4-c4nncn4C)c3)nc2c1] |
| 3 | H-1 | 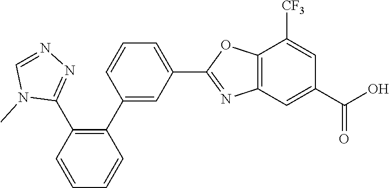 [SMILES: Cn1cnnc1-c1ccccc1-c1cccc(-c2nc3cc(C(=O)O)cc(C(F)(F)F)c3o2)c1] |
| 4 | I-2 | 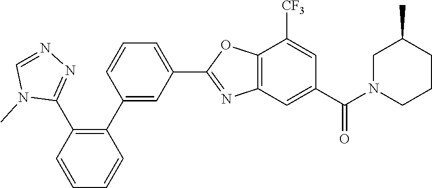 [SMILES: C[C@H]1CCCN(C(=O)c2cc(C(F)(F)F)c3oc(-c4cccc(-c5ccccc5-c5nncn5C)c4)nc3c2)C1] |
| 5 | J-1 | 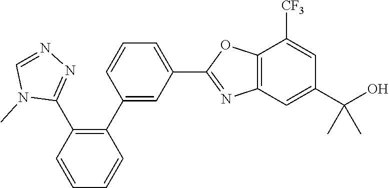 [SMILES: Cn1cnnc1-c1ccccc1-c1cccc(-c2nc3cc(C(C)(C)O)cc(C(F)(F)F)c3o2)c1] |
| 6 | K-2 | 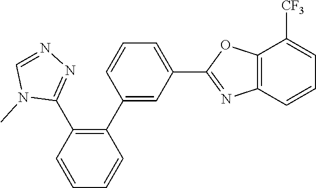 [SMILES: Cn1cnnc1-c1ccccc1-c1cccc(-c2nc3cccc(C(F)(F)F)c3o2)c1] |
| 7 | L-4 | 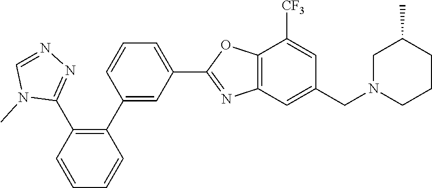 [SMILES: C[C@@H]1CCCN(Cc2cc(C(F)(F)F)c3oc(-c4cccc(-c5ccccc5-c5nncn5C)c4)nc3c2)C1] |
| 8 | M-5 | 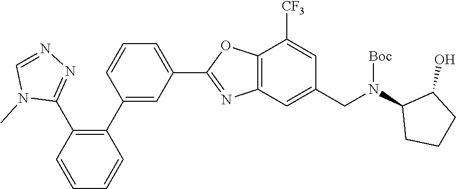 [SMILES: Cn1cnnc1-c1ccccc1-c1cccc(-c2nc3cc(CN(C(=O)OC(C)(C)C)[C@@H]4CCC[C@H]4O)cc(C(F)(F)F)c3o2)c1] |
| 9 | N-1 | 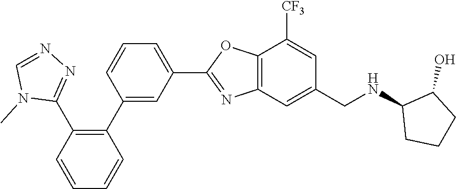 [SMILES: Cn1cnnc1-c1ccccc1-c1cccc(-c2nc3cc(CN[C@@H]4CCC[C@H]4O)cc(C(F)(F)F)c3o2)c1] |
| 10 | O-4 | 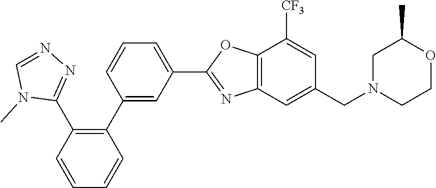 [SMILES: C[C@@H]1CN(Cc2cc(C(F)(F)F)c3oc(-c4cccc(-c5ccccc5-c5nncn5C)c4)nc3c2)CCO1] |
| 11 | P-6 | 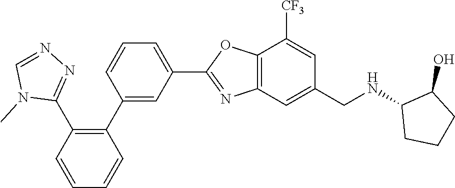 [SMILES: Cn1cnnc1-c1ccccc1-c1cccc(-c2nc3cc(CN[C@H]4CCC[C@@H]4O)cc(C(F)(F)F)c3o2)c1] |
| 12 | Q-3 | 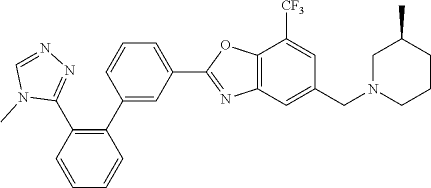 [SMILES: C[C@H]1CCCN(Cc2cc(C(F)(F)F)c3oc(-c4cccc(-c5ccccc5-c5nncn5C)c4)nc3c2)C1] |
| 13 | R-3 | 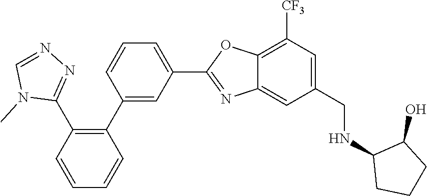 [SMILES: Cn1cnnc1-c1ccccc1-c1cccc(-c2nc3cc(CN[C@@H]4CCC[C@@H]4O)cc(C(F)(F)F)c3o2)c1] |
| 14 | S-1 | 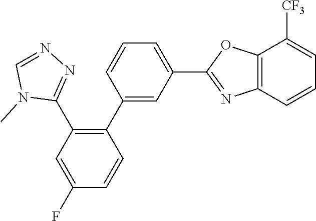 [SMILES: Cn1cnnc1-c1cc(F)ccc1-c1cccc(-c2nc3cccc(C(F)(F)F)c3o2)c1] |
| 15 | T-5 | 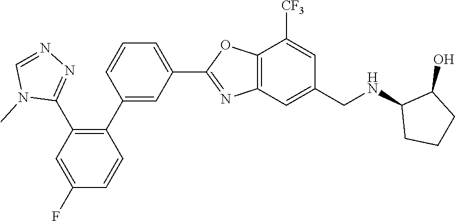 [SMILES: Cn1cnnc1-c1cc(F)ccc1-c1cccc(-c2nc3cc(CN[C@@H]4CCC[C@@H]4O)cc(C(F)(F)F)c3o2)c1] |
| 16 | U-1 | 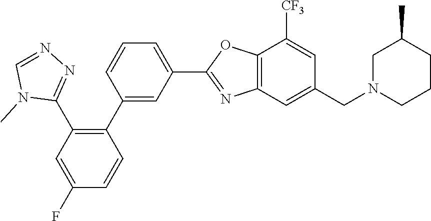 [SMILES: C[C@H]1CCCN(Cc2cc(C(F)(F)F)c3oc(-c4cccc(-c5ccc(F)cc5-c5nncn5C)c4)nc3c2)C1] |
| 17 | V-1 | 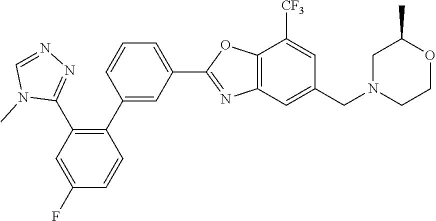 [SMILES: C[C@@H]1CN(Cc2cc(C(F)(F)F)c3oc(-c4cccc(-c5ccc(F)cc5-c5nncn5C)c4)nc3c2)CCO1] |
| 18 | W-2 | 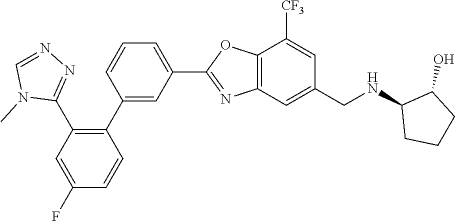 [SMILES: Cn1cnnc1-c1cc(F)ccc1-c1cccc(-c2nc3cc(CN[C@@H]4CCC[C@H]4O)cc(C(F)(F)F)c3o2)c1] |
| 19 | X-1 | 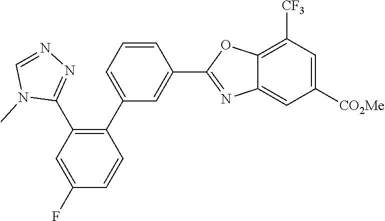 [SMILES: COC(=O)c1cc(C(F)(F)F)c2oc(-c3cccc(-c4ccc(F)cc4-c4nncn4C)c3)nc2c1] |
| 20 | X-2 | 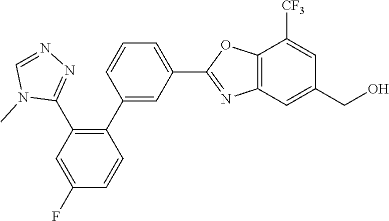 [SMILES: Cn1cnnc1-c1cc(F)ccc1-c1cccc(-c2nc3cc(CO)cc(C(F)(F)F)c3o2)c1] |
| 21 | Y-3 | 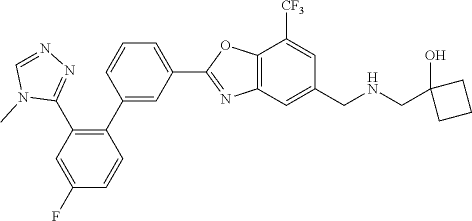 [SMILES: Cn1cnnc1-c1cc(F)ccc1-c1cccc(-c2nc3cc(CNCC4(O)CCC4)cc(C(F)(F)F)c3o2)c1] |
| 22 | Z-2 | 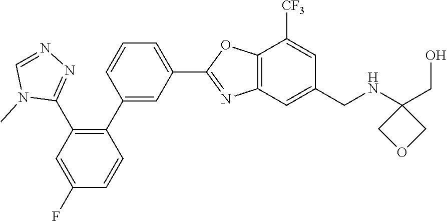 [SMILES: Cn1cnnc1-c1cc(F)ccc1-c1cccc(-c2nc3cc(CNC4(CO)COC4)cc(C(F)(F)F)c3o2)c1] |
| 23 | AA-2 | 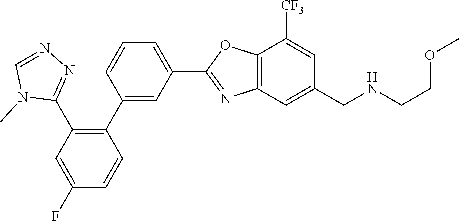 |
| 24 | AB-2 | 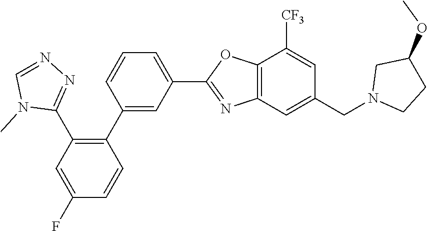 [SMILES: CO[C@H]1CCN(Cc2cc(C(F)(F)F)c3oc(-c4cccc(-c5ccc(F)cc5-c5nncn5C)c4)nc3c2)C1] |
| 25 | AC-2 | 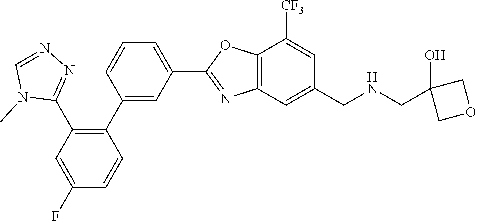 [SMILES: Cn1cnnc1-c1cc(F)ccc1-c1cccc(-c2nc3cc(CNCC4(O)COC4)cc(C(F)(F)F)c3o2)c1] |
| 26 | AD-2 | 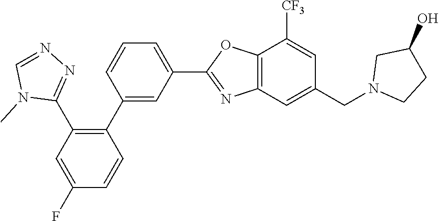 [SMILES: Cn1cnnc1-c1cc(F)ccc1-c1cccc(-c2nc3cc(CN4CC[C@H](O)C4)cc(C(F)(F)F)c3o2)c1] |
| 27 | AE-2 | 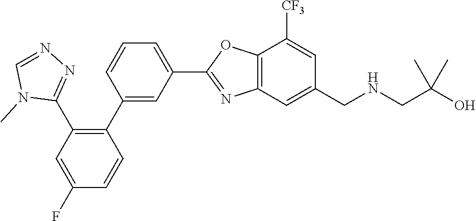 [SMILES: Cn1cnnc1-c1cc(F)ccc1-c1cccc(-c2nc3cc(CNCC(C)(C)O)cc(C(F)(F)F)c3o2)c1] |
| 28 | AF-2 | 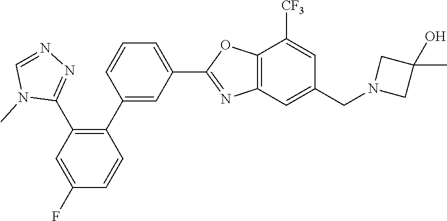 |
| 29 | AG-2 | 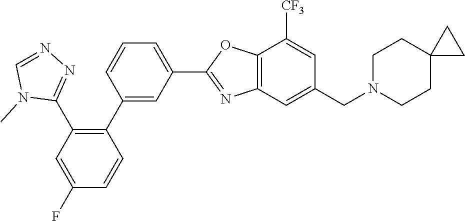 [SMILES: Cn1cnnc1-c1cc(F)ccc1-c1cccc(-c2nc3cc(CN4CCC5(CC4)CC5)cc(C(F)(F)F)c3o2)c1] |
| 30 | AH-2 | 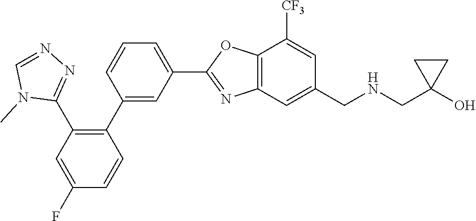 [SMILES: Cn1cnnc1-c1cc(F)ccc1-c1cccc(-c2nc3cc(CNCC4(O)CC4)cc(C(F)(F)F)c3o2)c1] |
| 31 | AI-2 | 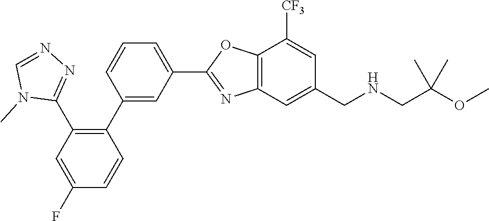 |
| 32 | AJ-3 | 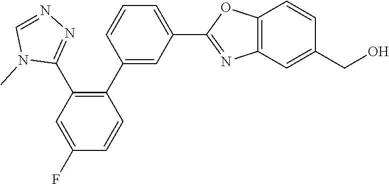 [SMILES: Cn1cnnc1-c1cc(F)ccc1-c1cccc(-c2nc3cc(CO)ccc3o2)c1] |
| 33 | AK-2 | 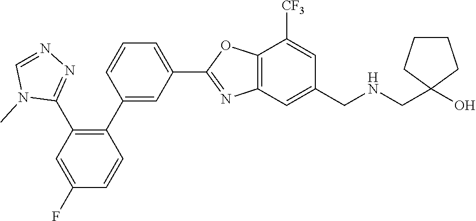 [SMILES: Cn1cnnc1-c1cc(F)ccc1-c1cccc(-c2nc3cc(CNCC4(O)CCCC4)cc(C(F)(F)F)c3o2)c1] |
| 34 | AL-2 | 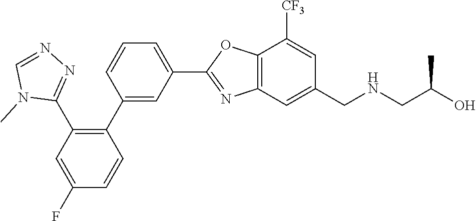 [SMILES: C[C@@H](O)CNCc1cc(C(F)(F)F)c2oc(-c3cccc(-c4ccc(F)cc4-c4nncn4C)c3)nc2c1] |
| 35 | AM-2 | 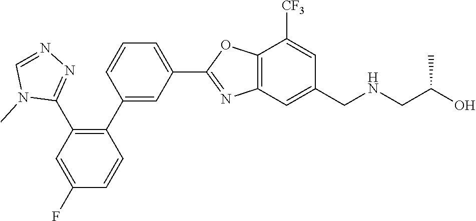 [SMILES: C[C@H](O)CNCc1cc(C(F)(F)F)c2oc(-c3cccc(-c4ccc(F)cc4-c4nncn4C)c3)nc2c1] |
| 36 | AN-2 | 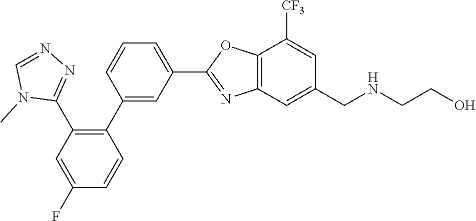 [SMILES: Cn1cnnc1-c1cc(F)ccc1-c1cccc(-c2nc3cc(CNCCO)cc(C(F)(F)F)c3o2)c1] |
| 37 | AO-2 | 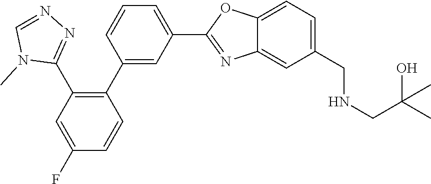 [SMILES: Cn1cnnc1-c1cc(F)ccc1-c1cccc(-c2nc3cc(CNCC(C)(C)O)ccc3o2)c1] |
| 38 | AP-2 | 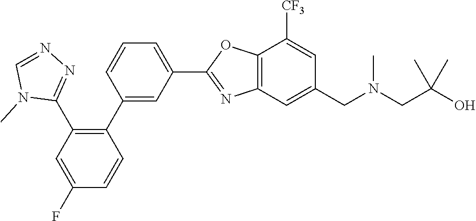 [SMILES: CN(Cc1cc(C(F)(F)F)c2oc(-c3cccc(-c4ccc(F)cc4-c4nncn4C)c3)nc2c1)CC(C)(C)O] |
| 39 | AQ-6 | 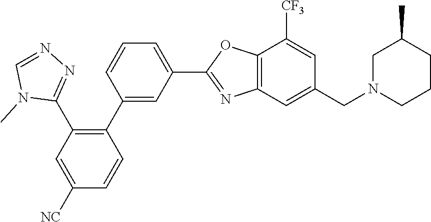 [SMILES: C[C@H]1CCCN(Cc2cc(C(F)(F)F)c3oc(-c4cccc(-c5ccc(C#N)cc5-c5nncn5C)c4)nc3c2)C1] |
| 40 | AR-2 | 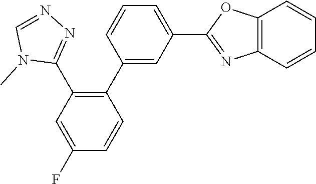 [SMILES: Cn1cnnc1-c1cc(F)ccc1-c1cccc(-c2nc3ccccc3o2)c1] |
| 41 | AS-12 | 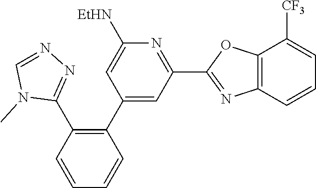 |
| 42 | AT-1 | 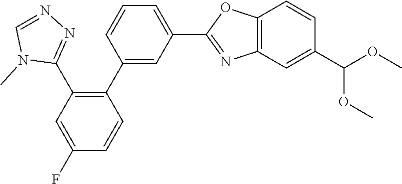 |
| 43 | AU-1 | 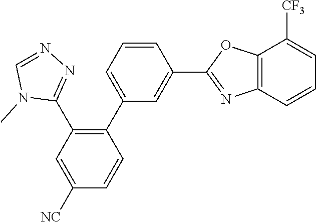 [SMILES: Cn1cnnc1-c1cc(C#N)ccc1-c1cccc(-c2nc3cccc(C(F)(F)F)c3o2)c1] |
| 44 | AV-9 | 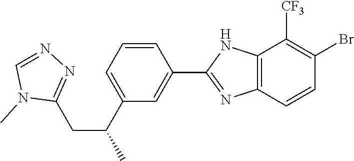 [SMILES: C[C@H](Cc1nncn1C)c1cccc(-c2nc3ccc(Br)c(C(F)(F)F)c3[nH]2)c1] |
| 45 | AV-10 | 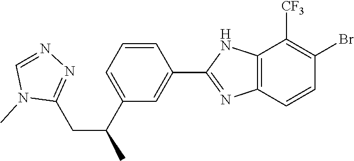 [SMILES: C[C@@H](Cc1nncn1C)c1cccc(-c2nc3ccc(Br)c(C(F)(F)F)c3[nH]2)c1] |
| 46 | AW-4 | 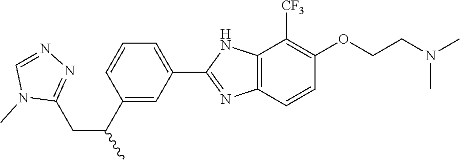 |
| 47 | AX-3 | 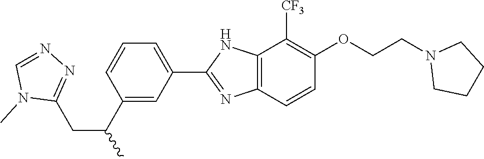 [SMILES: CC(Cc1nncn1C)c1cccc(-c2nc3ccc(OCCN4CCCC4)c(C(F)(F)F)c3[nH]2)c1] |
| 48 | AY-5 | 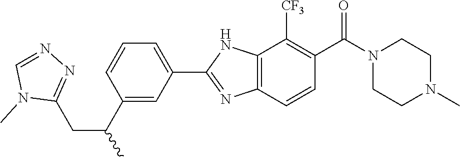 |
| 49 | AZ-1 | 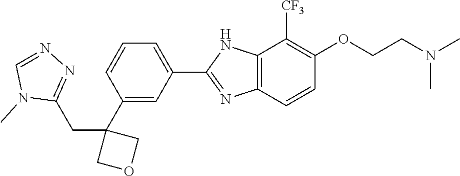 |
| 50 | BA-1 | 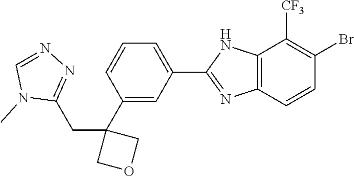 [SMILES: Cn1cnnc1CC1(c2cccc(-c3nc4ccc(Br)c(C(F)(F)F)c4[nH]3)c2)COC1] |
| 51 | BB-1 | 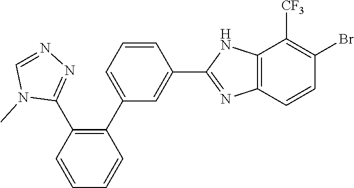 [SMILES: Cn1cnnc1-c1ccccc1-c1cccc(-c2nc3ccc(Br)c(C(F)(F)F)c3[nH]2)c1] |
| 52 | BC-1 | 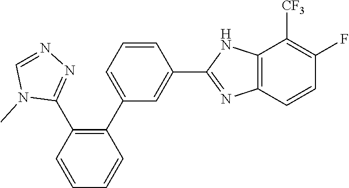 [SMILES: Cn1cnnc1-c1ccccc1-c1cccc(-c2nc3ccc(F)c(C(F)(F)F)c3[nH]2)c1] |
| 53 | BD-2 | 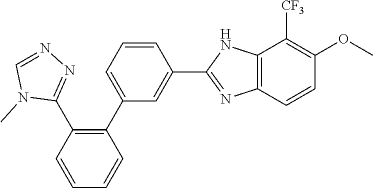 |
| 54 | BE-2 | 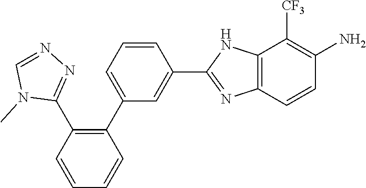 [SMILES: Cn1cnnc1-c1ccccc1-c1cccc(-c2nc3ccc(N)c(C(F)(F)F)c3[nH]2)c1] |
| 55 | BF-3 | 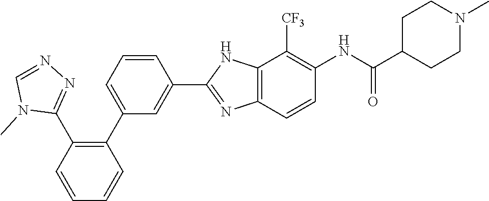 |
| 56 | BG-1 | 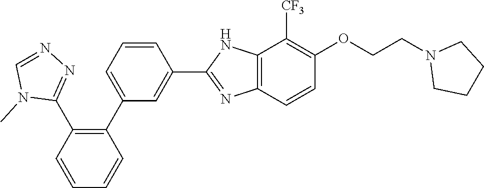 [SMILES: Cn1cnnc1-c1ccccc1-c1cccc(-c2nc3ccc(OCCN4CCCC4)c(C(F)(F)F)c3[nH]2)c1] |
| 57 | BH-3 | 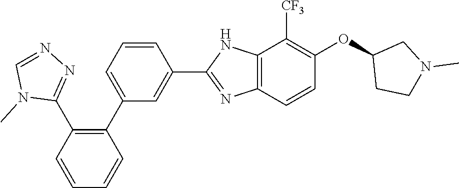 |
| 58 | BI-1 | 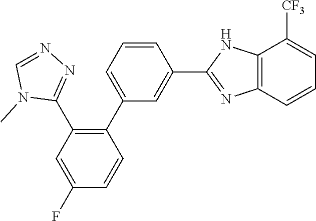 [SMILES: Cn1cnnc1-c1cc(F)ccc1-c1cccc(-c2nc3cccc(C(F)(F)F)c3[nH]2)c1] |
| 59 | BJ-1 | 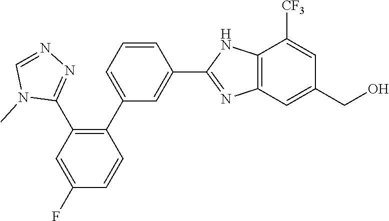 [SMILES: Cn1cnnc1-c1cc(F)ccc1-c1cccc(-c2nc3cc(CO)cc(C(F)(F)F)c3[nH]2)c1] |
| 60 | BK-2 | 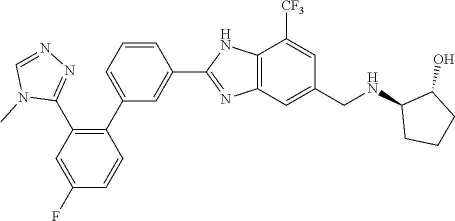 [SMILES: Cn1cnnc1-c1cc(F)ccc1-c1cccc(-c2nc3cc(CN[C@@H]4CCC[C@H]4O)cc(C(F)(F)F)c3[nH]2)c1] |
| 61 | BL-2 | 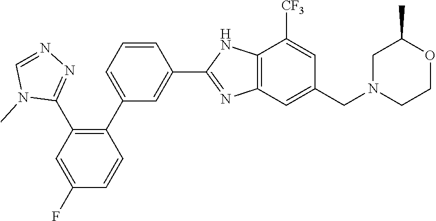 [SMILES: C[C@@H]1CN(Cc2cc(C(F)(F)F)c3[nH]c(-c4cccc(-c5ccc(F)cc5-c5nncn5C)c4)nc3c2)CCO1] |
| 62 | BM-2 | 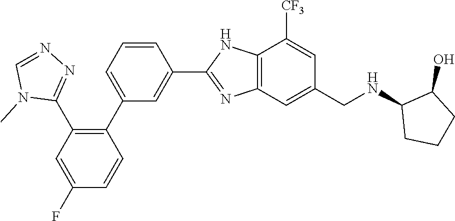 [SMILES: Cn1cnnc1-c1cc(F)ccc1-c1cccc(-c2nc3cc(CN[C@@H]4CCC[C@@H]4O)cc(C(F)(F)F)c3[nH]2)c1] |
| 63 | BN-2 | 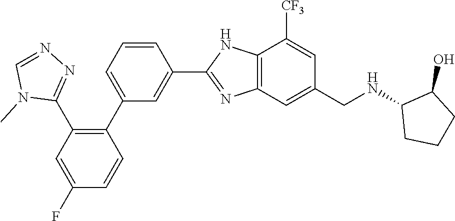 [SMILES: Cn1cnnc1-c1cc(F)ccc1-c1cccc(-c2nc3cc(CN[C@H]4CCC[C@@H]4O)cc(C(F)(F)F)c3[nH]2)c1] |
| 64 | BO-2 | 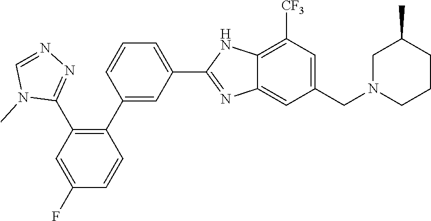 [SMILES: C[C@H]1CCCN(Cc2cc(C(F)(F)F)c3[nH]c(-c4cccc(-c5ccc(F)cc5-c5nncn5C)c4)nc3c2)C1] |
| 65 | BP-1 | 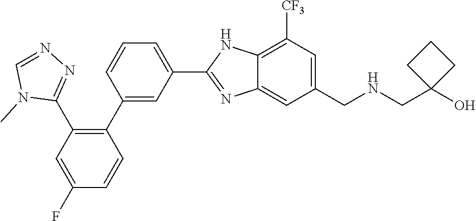 [SMILES: Cn1cnnc1-c1cc(F)ccc1-c1cccc(-c2nc3cc(CNCC4(O)CCC4)cc(C(F)(F)F)c3[nH]2)c1] |
| 66 | BQ-1 | 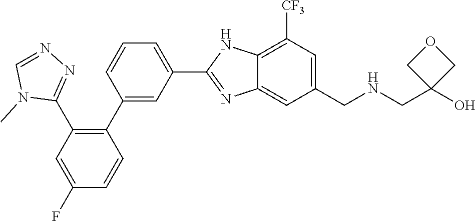 [SMILES: Cn1cnnc1-c1cc(F)ccc1-c1cccc(-c2nc3cc(CNCC4(O)COC4)cc(C(F)(F)F)c3[nH]2)c1] |
| 67 | BR-1 | 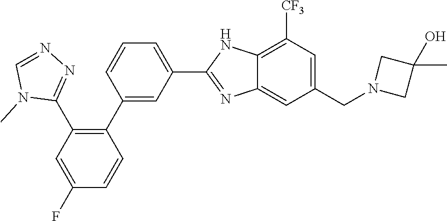 |
| 68 | BS-1 | 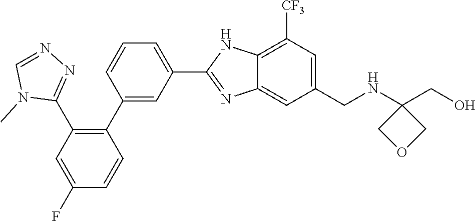 [SMILES: Cn1cnnc1-c1cc(F)ccc1-c1cccc(-c2nc3cc(CNC4(CO)COC4)cc(C(F)(F)F)c3[nH]2)c1] |
| 69 | BT-1 | 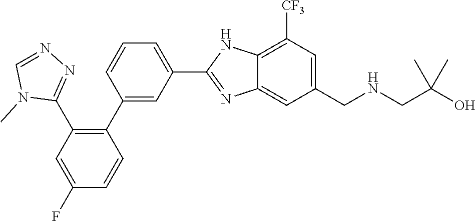 [SMILES: Cn1cnnc1-c1cc(F)ccc1-c1cccc(-c2nc3cc(CNCC(C)(C)O)cc(C(F)(F)F)c3[nH]2)c1] |
| 70 | BU-1 | 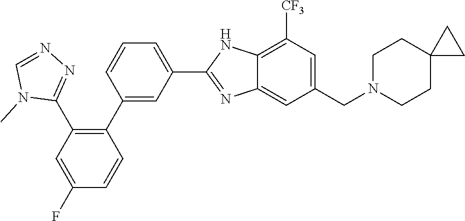 [SMILES: Cn1cnnc1-c1cc(F)ccc1-c1cccc(-c2nc3cc(CN4CCC5(CC4)CC5)cc(C(F)(F)F)c3[nH]2)c1] |
| 71 | BV-1 | 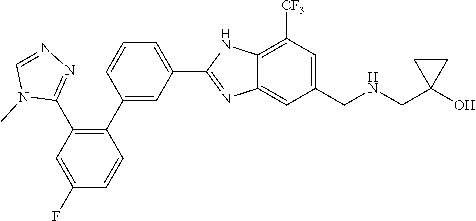 [SMILES: Cn1cnnc1-c1cc(F)ccc1-c1cccc(-c2nc3cc(CNCC4(O)CC4)cc(C(F)(F)F)c3[nH]2)c1] |
| 72 | BW-3 | 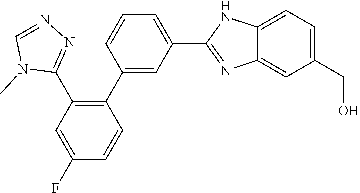 [SMILES: Cn1cnnc1-c1cc(F)ccc1-c1cccc(-c2nc3cc(CO)ccc3[nH]2)c1] |
| 73 | BX-2 | 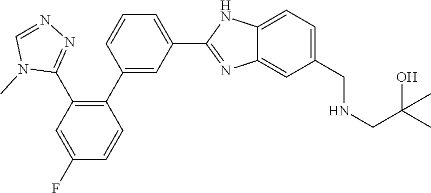 [SMILES: Cn1cnnc1-c1cc(F)ccc1-c1cccc(-c2nc3cc(CNCC(C)(C)O)ccc3[nH]2)c1] |
| 74 | BY-1 | 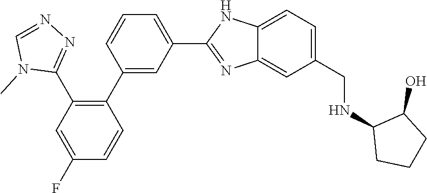 [SMILES: Cn1cnnc1-c1cc(F)ccc1-c1cccc(-c2nc3cc(CN[C@@H]4CCC[C@@H]4O)ccc3[nH]2)c1] |
| 75 | BZ-1 | 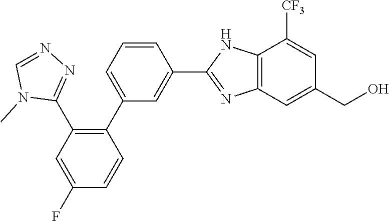 [SMILES: Cn1cnnc1-c1cc(F)ccc1-c1cccc(-c2nc3cc(CO)cc(C(F)(F)F)c3[nH]2)c1] |
| 76 | CA-2 | 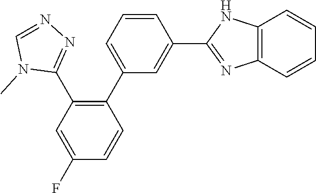 [SMILES: Cn1cnnc1-c1cc(F)ccc1-c1cccc(-c2nc3ccccc3[nH]2)c1] |
| 77 | CB-1 | 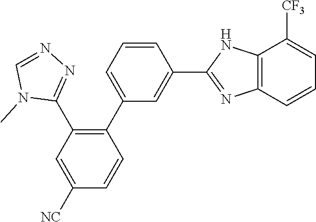 [SMILES: Cn1cnnc1-c1cc(C#N)ccc1-c1cccc(-c2nc3cccc(C(F)(F)F)c3[nH]2)c1] |
| 78 | CC-2 | 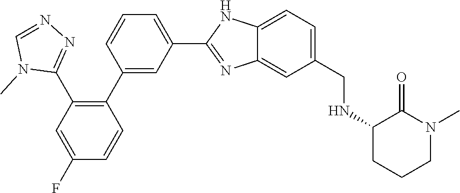 |
| 79 | CD-2 | 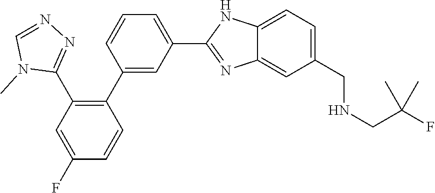 [SMILES: Cn1cnnc1-c1cc(F)ccc1-c1cccc(-c2nc3cc(CNCC(C)(C)F)ccc3[nH]2)c1] |
| 80 | CE-4 | 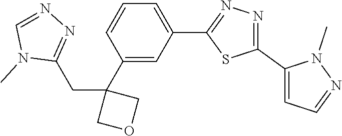 [SMILES: Cn1cnnc1CC1(c2cccc(-c3nnc(-c4ccnn4C)s3)c2)COC1] |
| 81 | CF-2 | 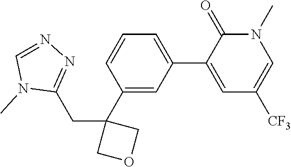 [SMILES: Cn1cnnc1CC1(c2cccc(-c3cc(C(F)(F)F)cn(C)c3=O)c2)COC1] |
| 82 | CG-4 | 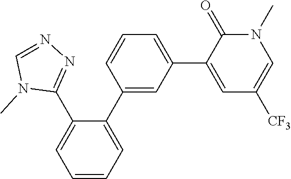 [SMILES: Cn1cnnc1-c1ccccc1-c1cccc(-c2cc(C(F)(F)F)cn(C)c2=O)c1] |
| 83 | CH-2 | 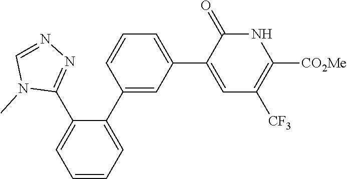 [SMILES: COC(=O)c1[nH]c(=O)c(-c2cccc(-c3ccccc3-c3nncn3C)c2)cc1C(F)(F)F] |
| 84 | CH-3 | 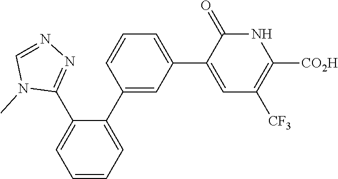 [SMILES: Cn1cnnc1-c1ccccc1-c1cccc(-c2cc(C(F)(F)F)c(C(=O)O)[nH]c2=O)c1] |
| 85 | CI-4 | 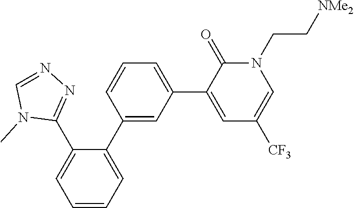 [SMILES: CN(C)CCn1cc(C(F)(F)F)cc(-c2cccc(-c3ccccc3-c3nncn3C)c2)c1=O] |
| 86 | CJ-1 | 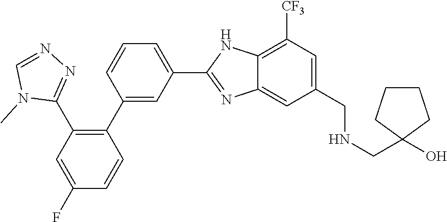 [SMILES: Cn1cnnc1-c1cc(F)ccc1-c1cccc(-c2nc3cc(CNCC4(O)CCCC4)cc(C(F)(F)F)c3[nH]2)c1] |
| 87 | CK-2 | 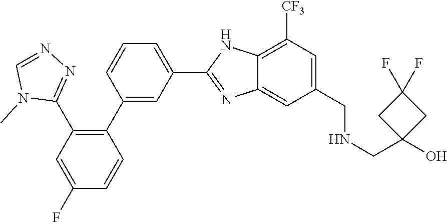 [SMILES: Cn1cnnc1-c1cc(F)ccc1-c1cccc(-c2nc3cc(CNCC4(O)CC(F)(F)C4)cc(C(F)(F)F)c3[nH]2)c1] |
| 88 | CL-2 | 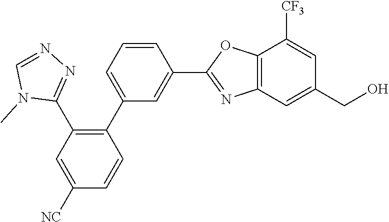 [SMILES: Cn1cnnc1-c1cc(C#N)ccc1-c1cccc(-c2nc3cc(CO)cc(C(F)(F)F)c3o2)c1] |
| 89 | CM-1 | 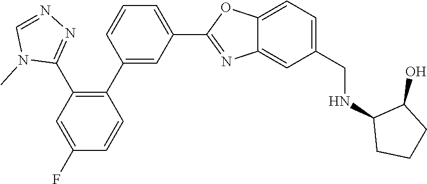 [SMILES: Cn1cnnc1-c1cc(F)ccc1-c1cccc(-c2nc3cc(CN[C@@H]4CCC[C@@H]4O)ccc3o2)c1] |
| 90 | CN-5 | 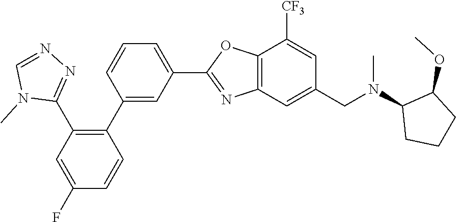 [SMILES: CO[C@H]1CCC[C@H]1N(C)Cc1cc(C(F)(F)F)c2oc(-c3cccc(-c4ccc(F)cc4-c4nncn4C)c3)nc2c1] |
| 91 | CO-2 | 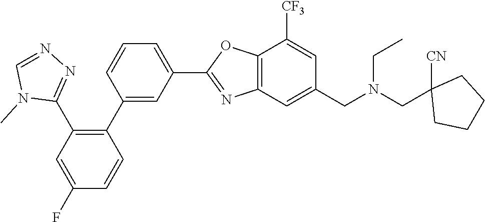 [SMILES: CCN(Cc1cc(C(F)(F)F)c2oc(-c3cccc(-c4ccc(F)cc4-c4nncn4C)c3)nc2c1)CC1(C#N)CCCC1] |
| 92 | CP-4 | 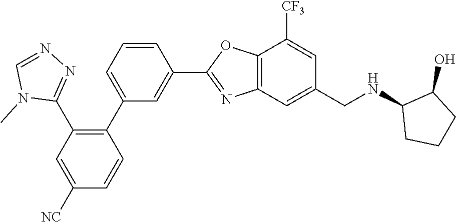 [SMILES: Cn1cnnc1-c1cc(C#N)ccc1-c1cccc(-c2nc3cc(CN[C@@H]4CCC[C@@H]4O)cc(C(F)(F)F)c3o2)c1] |
| 93 | CQ-1 | 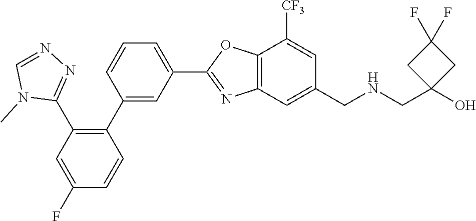 [SMILES: Cn1cnnc1-c1cc(F)ccc1-c1cccc(-c2nc3cc(CNCC4(O)CC(F)(F)C4)cc(C(F)(F)F)c3o2)c1] |
| 94 | CR-2 | 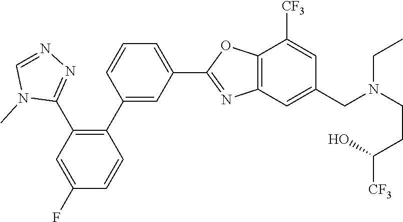 [SMILES: CCN(CC[C@@H](O)C(F)(F)F)Cc1cc(C(F)(F)F)c2oc(-c3cccc(-c4ccc(F)cc4-c4nncn4C)c3)nc2c1] |
| 95 | CR-3 | 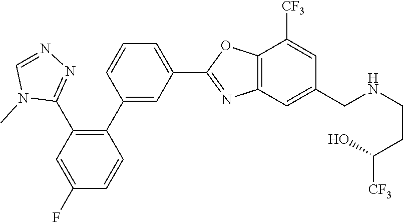 [SMILES: Cn1cnnc1-c1cc(F)ccc1-c1cccc(-c2nc3cc(CNCC[C@@H](O)C(F)(F)F)cc(C(F)(F)F)c3o2)c1] |
| 96 | CS-1 | 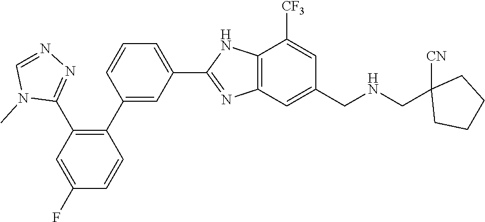 [SMILES: Cn1cnnc1-c1cc(F)ccc1-c1cccc(-c2nc3cc(CNCC4(C#N)CCCC4)cc(C(F)(F)F)c3[nH]2)c1] |
| 97 | CT-2 | 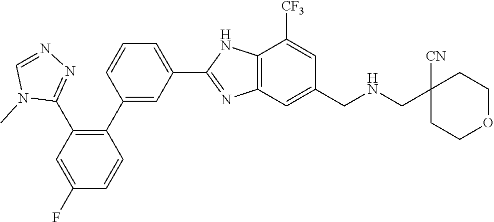 [SMILES: Cn1cnnc1-c1cc(F)ccc1-c1cccc(-c2nc3cc(CNCC4(C#N)CCOCC4)cc(C(F)(F)F)c3[nH]2)c1] |
| 98 | CU-2 | 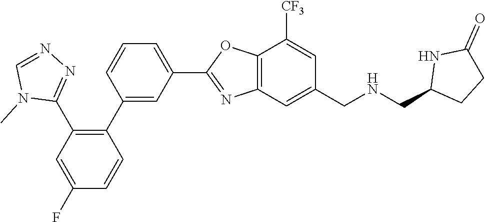 [SMILES: Cn1cnnc1-c1cc(F)ccc1-c1cccc(-c2nc3cc(CNC[C@@H]4CCC(=O)N4)cc(C(F)(F)F)c3o2)c1] |
| 99 | CV-2 | 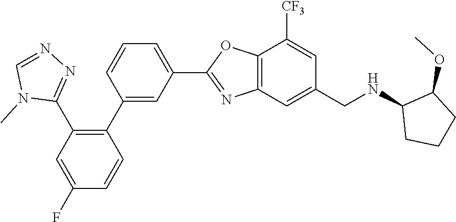 [SMILES: CO[C@H]1CCC[C@H]1NCc1cc(C(F)(F)F)c2oc(-c3cccc(-c4ccc(F)cc4-c4nncn4C)c3)nc2c1] |
| 100 | CW-5 | 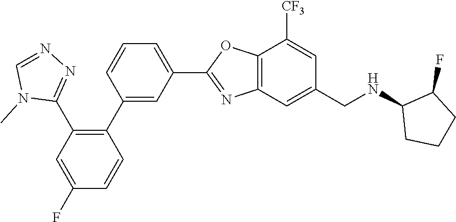 [SMILES: Cn1cnnc1-c1cc(F)ccc1-c1cccc(-c2nc3cc(CN[C@@H]4CCC[C@@H]4F)cc(C(F)(F)F)c3o2)c1] |
| 101 | CX-1 | 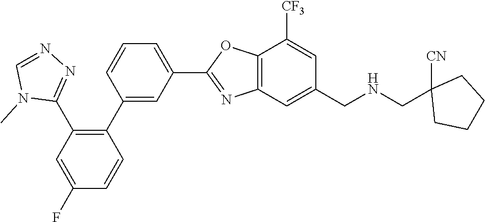 [SMILES: Cn1cnnc1-c1cc(F)ccc1-c1cccc(-c2nc3cc(CNCC4(C#N)CCCC4)cc(C(F)(F)F)c3o2)c1] |
| 102 | CY-1 | 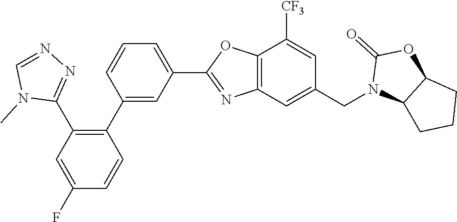 [SMILES: Cn1cnnc1-c1cc(F)ccc1-c1cccc(-c2nc3cc(CN4C(=O)O[C@H]5CCC[C@H]54)cc(C(F)(F)F)c3o2)c1] |
| 103 | CZ-2 | 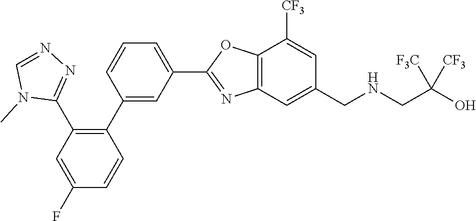 [SMILES: Cn1cnnc1-c1cc(F)ccc1-c1cccc(-c2nc3cc(CNCC(O)(C(F)(F)F)C(F)(F)F)cc(C(F)(F)F)c3o2)c1] |
| 104 | DA-1 | 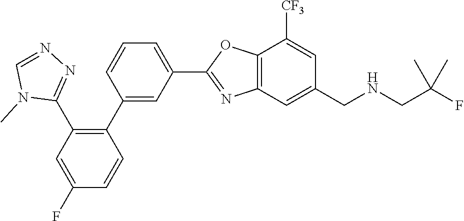 [SMILES: Cn1cnnc1-c1cc(F)ccc1-c1cccc(-c2nc3cc(CNCC(C)(C)F)cc(C(F)(F)F)c3o2)c1] |
| 105 | DB-2 | 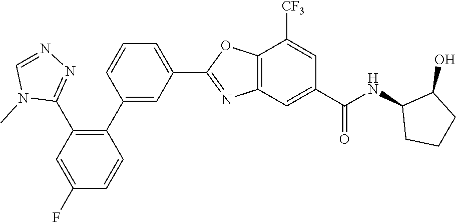 [SMILES: Cn1cnnc1-c1cc(F)ccc1-c1cccc(-c2nc3cc(C(=O)N[C@@H]4CCC[C@@H]4O)cc(C(F)(F)F)c3o2)c1] |
| 106 | DC-3 | 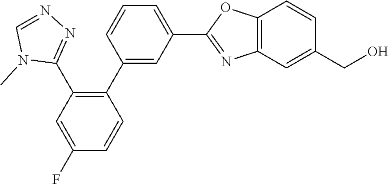 [SMILES: Cn1cnnc1-c1cc(F)ccc1-c1cccc(-c2nc3cc(CO)ccc3o2)c1] |
| 107 | DD-2 | 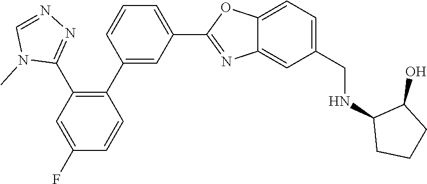 [SMILES: Cn1cnnc1-c1cc(F)ccc1-c1cccc(-c2nc3cc(CN[C@@H]4CCC[C@@H]4O)ccc3o2)c1] |
| 108 | DE-2 | 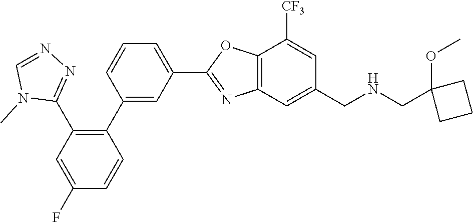 [SMILES: COC1(CNCc2cc(C(F)(F)F)c3oc(-c4cccc(-c5ccc(F)cc5-c5nncn5C)c4)nc3c2)CCC1] |
| 109 | DF-2 | 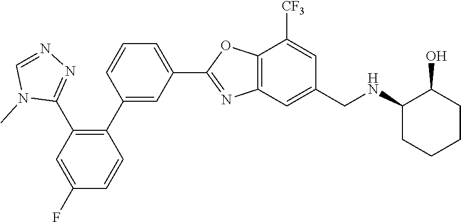 [SMILES: Cn1cnnc1-c1cc(F)ccc1-c1cccc(-c2nc3cc(CN[C@@H]4CCCC[C@@H]4O)cc(C(F)(F)F)c3o2)c1] |
| 110 | DG-2 | 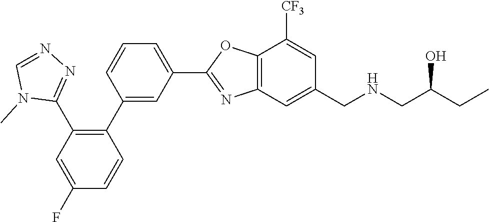 [SMILES: CC[C@H](O)CNCc1cc(C(F)(F)F)c2oc(-c3cccc(-c4ccc(F)cc4-c4nncn4C)c3)nc2c1] |
| 111 | DH-2 | 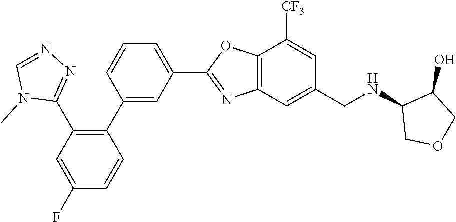 [SMILES: Cn1cnnc1-c1cc(F)ccc1-c1cccc(-c2nc3cc(CN[C@@H]4COC[C@@H]4O)cc(C(F)(F)F)c3o2)c1] |
| 112 | DI-1 | 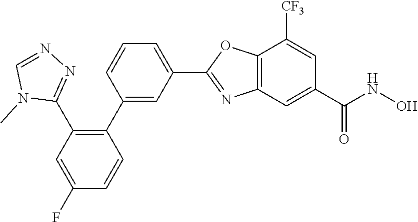 [SMILES: Cn1cnnc1-c1cc(F)ccc1-c1cccc(-c2nc3cc(C(=O)NO)cc(C(F)(F)F)c3o2)c1] |
| 113 | DJ-2 | 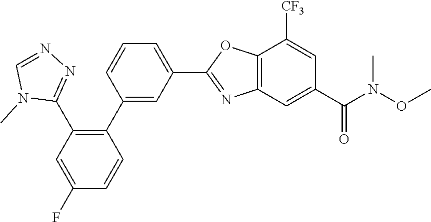 |
| 114 | DK-1 | 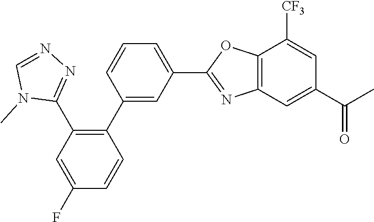 [SMILES: CC(=O)c1cc(C(F)(F)F)c2oc(-c3cccc(-c4ccc(F)cc4-c4nncn4C)c3)nc2c1] |
| 115 | DL-1 | 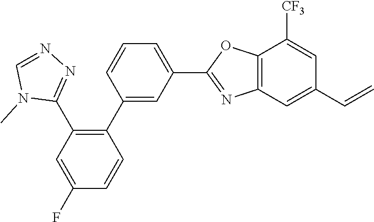 [SMILES: C=Cc1cc(C(F)(F)F)c2oc(-c3cccc(-c4ccc(F)cc4-c4nncn4C)c3)nc2c1] |
| 116 | DM-2 | 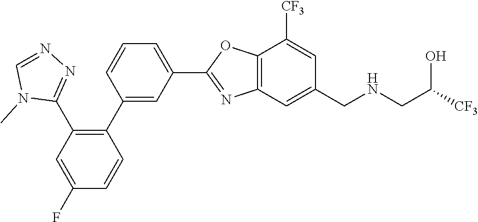 |
| 117 | DN-2 | 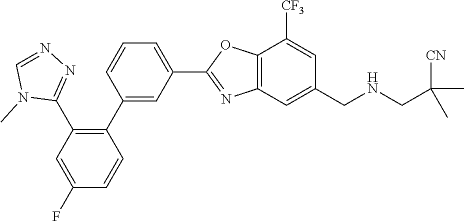 [SMILES: Cn1cnnc1-c1cc(F)ccc1-c1cccc(-c2nc3cc(CNCC(C)(C)C#N)cc(C(F)(F)F)c3o2)c1] |
| 118 | DO-6 | 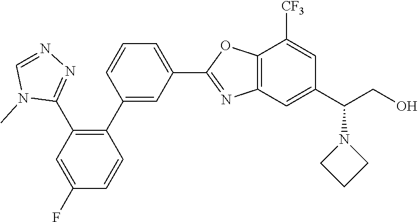 [SMILES: Cn1cnnc1-c1cc(F)ccc1-c1cccc(-c2nc3cc([C@H](CO)N4CCC4)cc(C(F)(F)F)c3o2)c1] |
| 119 | DO-7 | 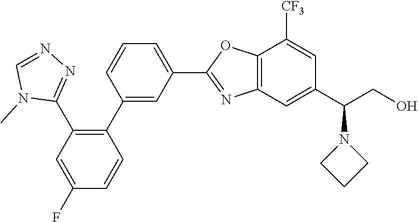 [SMILES: Cn1cnnc1-c1cc(F)ccc1-c1cccc(-c2nc3cc([C@@H](CO)N4CCC4)cc(C(F)(F)F)c3o2)c1] |
| 120 | DP-5 | 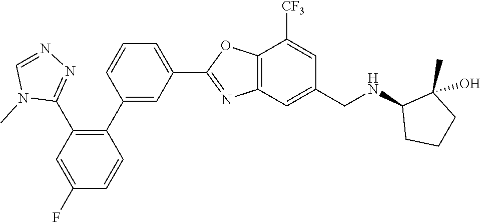 [SMILES: Cn1cnnc1-c1cc(F)ccc1-c1cccc(-c2nc3cc(CN[C@@H]4CCC[C@@]4(C)O)cc(C(F)(F)F)c3o2)c1] |
| 121 | DP-6 | 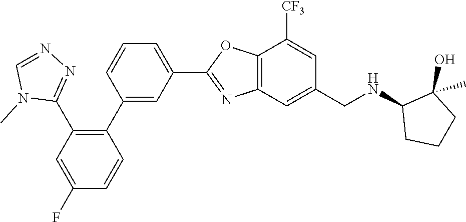 |
| 122 | DQ-1 | 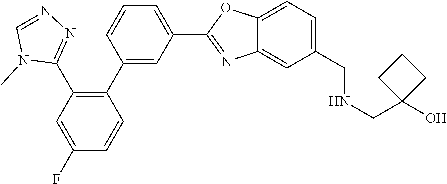 [SMILES: Cn1cnnc1-c1cc(F)ccc1-c1cccc(-c2nc3cc(CNCC4(O)CCC4)ccc3o2)c1] |
| 123 | DR-2 | 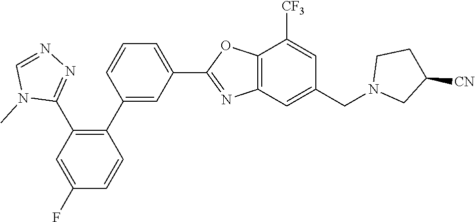 [SMILES: Cn1cnnc1-c1cc(F)ccc1-c1cccc(-c2nc3cc(CN4CC[C@@H](C#N)C4)cc(C(F)(F)F)c3o2)c1] |
| 124 | DS-2 | 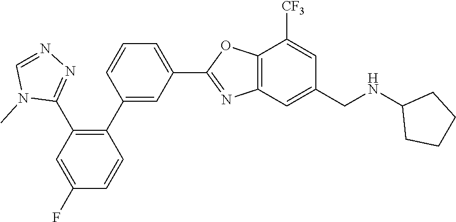 [SMILES: Cn1cnnc1-c1cc(F)ccc1-c1cccc(-c2nc3cc(CNC4CCCC4)cc(C(F)(F)F)c3o2)c1] |
| 125 | DT-2 | 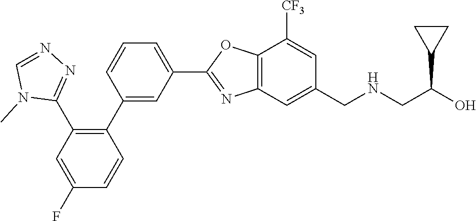 [SMILES: Cn1cnnc1-c1cc(F)ccc1-c1cccc(-c2nc3cc(CNC[C@H](O)C4CC4)cc(C(F)(F)F)c3o2)c1] |
| 126 | DT-3 | 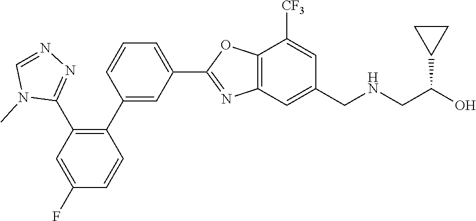 [SMILES: Cn1cnnc1-c1cc(F)ccc1-c1cccc(-c2nc3cc(CNC[C@@H](O)C4CC4)cc(C(F)(F)F)c3o2)c1] |
| 127 | DU-2 | 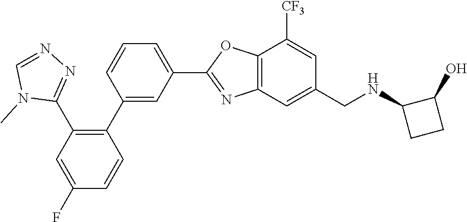 [SMILES: Cn1cnnc1-c1cc(F)ccc1-c1cccc(-c2nc3cc(CN[C@@H]4CC[C@@H]4O)cc(C(F)(F)F)c3o2)c1] |
| 128 | DV-2 | 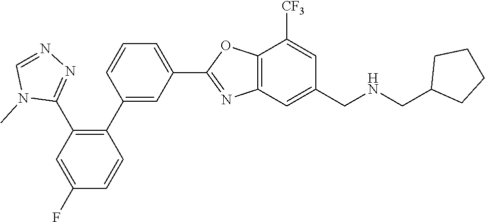 [SMILES: Cn1cnnc1-c1cc(F)ccc1-c1cccc(-c2nc3cc(CNCC4CCCC4)cc(C(F)(F)F)c3o2)c1] |
| 129 | DW-3 | 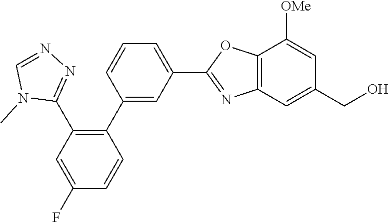 [SMILES: COc1cc(CO)cc2nc(-c3cccc(-c4ccc(F)cc4-c4nncn4C)c3)oc12] |
| 130 | DX-2 | 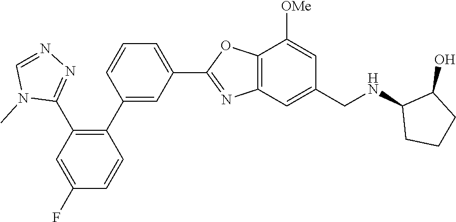 [SMILES: COc1cc(CN[C@@H]2CCC[C@@H]2O)cc2nc(-c3cccc(-c4ccc(F)cc4-c4nncn4C)c3)oc12] |
| 131 | DY-1 | 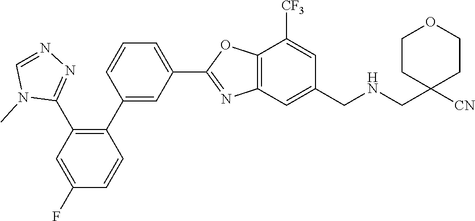 [SMILES: Cn1cnnc1-c1cc(F)ccc1-c1cccc(-c2nc3cc(CNCC4(C#N)CCOCC4)cc(C(F)(F)F)c3o2)c1] |
| 132 | DZ-1 | 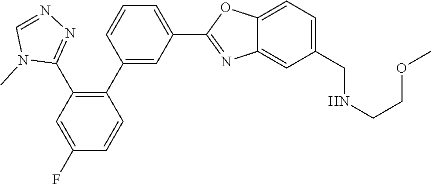 |
| 133 | EA-1 | 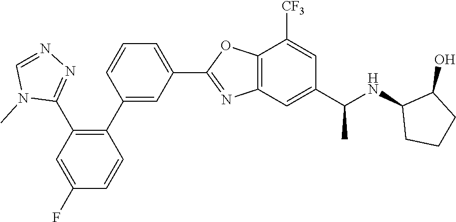 [SMILES: C[C@H](N[C@@H]1CCC[C@@H]1O)c1cc(C(F)(F)F)c2oc(-c3cccc(-c4ccc(F)cc4-c4nncn4C)c3)nc2c1] |
| 134 | EA-2 | 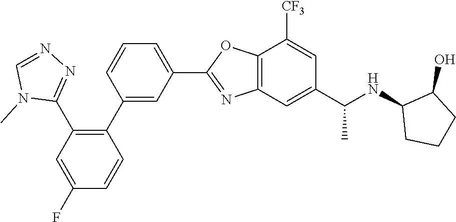 [SMILES: C[C@@H](N[C@@H]1CCC[C@@H]1O)c1cc(C(F)(F)F)c2oc(-c3cccc(-c4ccc(F)cc4-c4nncn4C)c3)nc2c1] |
| 135 | EB-1 | 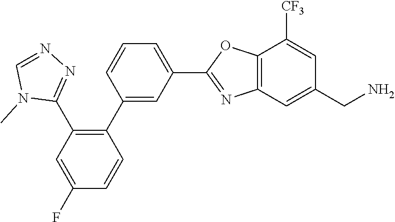 [SMILES: Cn1cnnc1-c1cc(F)ccc1-c1cccc(-c2nc3cc(CN)cc(C(F)(F)F)c3o2)c1] |
| 136 | EC-1 | 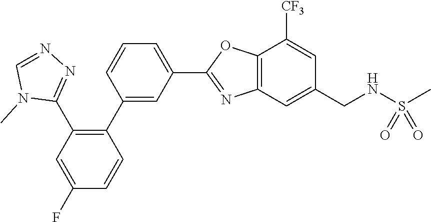 |
| 137 | ED-7 | 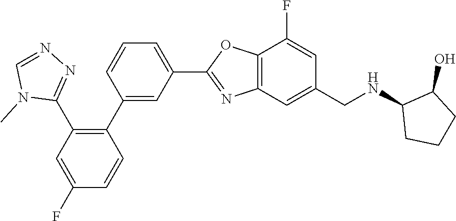 [SMILES: Cn1cnnc1-c1cc(F)ccc1-c1cccc(-c2nc3cc(CN[C@@H]4CCC[C@@H]4O)cc(F)c3o2)c1] |
| 138 | EE-2 | 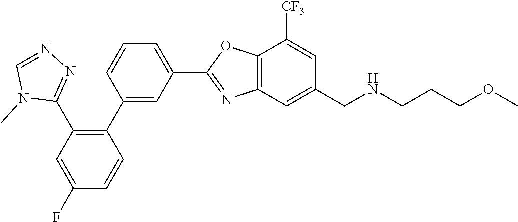 |
| 139 | EF-1 | 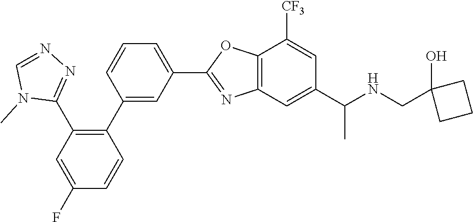 [SMILES: CC(NCC1(O)CCC1)c1cc(C(F)(F)F)c2oc(-c3cccc(-c4ccc(F)cc4-c4nncn4C)c3)nc2c1] |
| 140 | EG-2 | 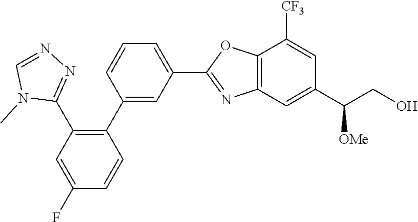 [SMILES: CO[C@H](CO)c1cc(C(F)(F)F)c2oc(-c3cccc(-c4ccc(F)cc4-c4nncn4C)c3)nc2c1] |
| 141 | EG-3 | 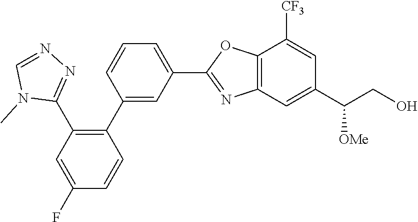 [SMILES: CO[C@@H](CO)c1cc(C(F)(F)F)c2oc(-c3cccc(-c4ccc(F)cc4-c4nncn4C)c3)nc2c1] |
| 142 | EH-1 | 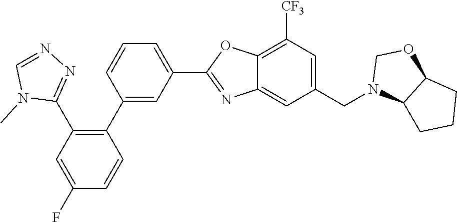 [SMILES: Cn1cnnc1-c1cc(F)ccc1-c1cccc(-c2nc3cc(CN4CO[C@H]5CCC[C@H]54)cc(C(F)(F)F)c3o2)c1] |
| 143 | EI-4 | 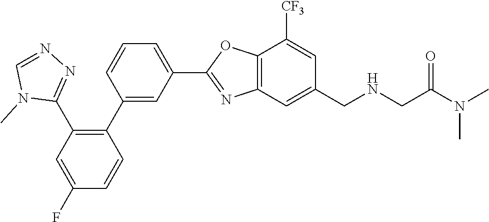 |
| 144 | EJ-2 | 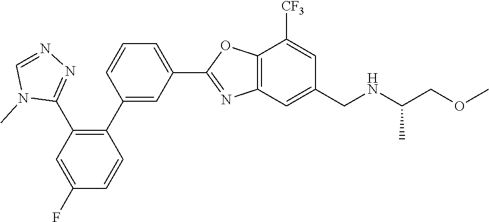 |
| 145 | EK-5 | 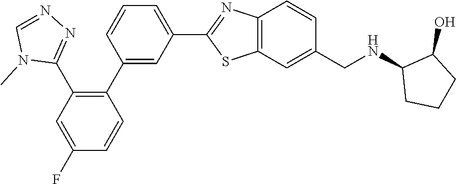 [SMILES: Cn1cnnc1-c1cc(F)ccc1-c1cccc(-c2nc3ccc(CN[C@@H]4CCC[C@@H]4O)cc3s2)c1] |
| 146 | EL-1 | 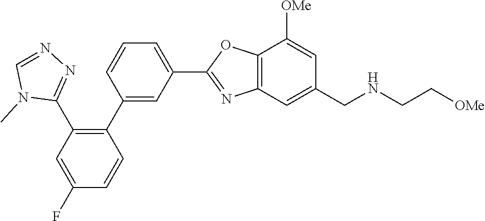 [SMILES: COCCNCc1cc(OC)c2oc(-c3cccc(-c4ccc(F)cc4-c4nncn4C)c3)nc2c1] |
| 147 | EM-2 | 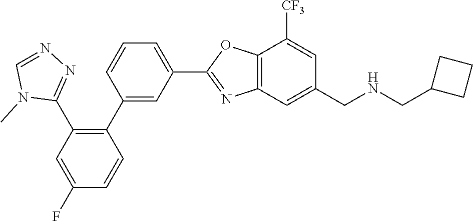 [SMILES: Cn1cnnc1-c1cc(F)ccc1-c1cccc(-c2nc3cc(CNCC4CCC4)cc(C(F)(F)F)c3o2)c1] |
| 148 | EN-2 | 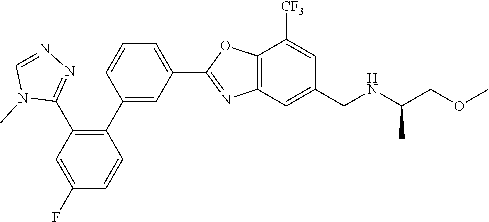 |
| 149 | EO-2 | 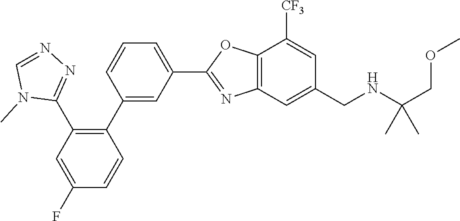 |
| 150 | EP-2 | 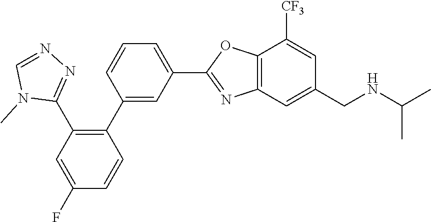 [SMILES: CC(C)NCc1cc(C(F)(F)F)c2oc(-c3cccc(-c4ccc(F)cc4-c4nncn4C)c3)nc2c1] |
| 151 | EQ-4 | 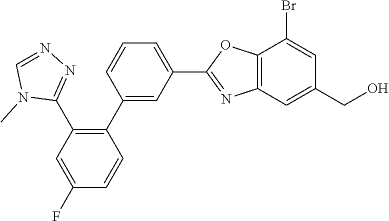 [SMILES: Cn1cnnc1-c1cc(F)ccc1-c1cccc(-c2nc3cc(CO)cc(Br)c3o2)c1] |
| 152 | ER-2 | 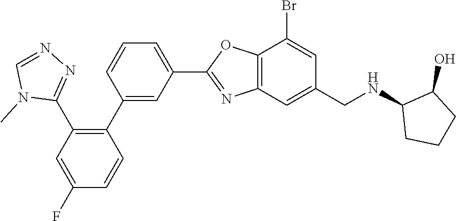 [SMILES: Cn1cnnc1-c1cc(F)ccc1-c1cccc(-c2nc3cc(CN[C@@H]4CCC[C@@H]4O)cc(Br)c3o2)c1] |
| 153 | ES-4 | 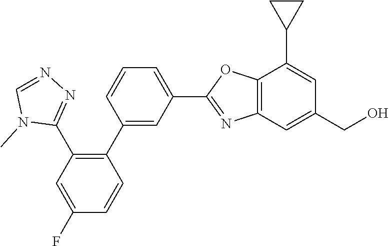 [SMILES: Cn1cnnc1-c1cc(F)ccc1-c1cccc(-c2nc3cc(CO)cc(C4CC4)c3o2)c1] |
| 154 | ES-6 | 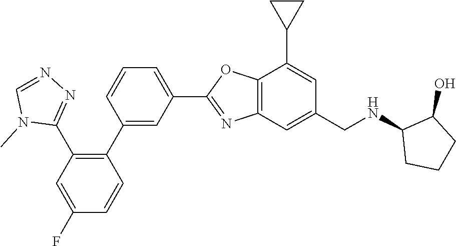 [SMILES: Cn1cnnc1-c1cc(F)ccc1-c1cccc(-c2nc3cc(CN[C@@H]4CCC[C@@H]4O)cc(C4CC4)c3o2)c1] |
| 155 | ET-8 | 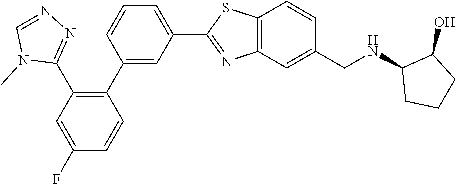 [SMILES: Cn1cnnc1-c1cc(F)ccc1-c1cccc(-c2nc3cc(CN[C@@H]4CCC[C@@H]4O)ccc3s2)c1] |
| 156 | EU-2 | 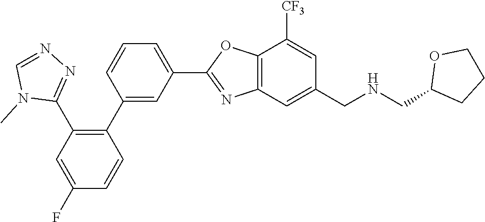 [SMILES: Cn1cnnc1-c1cc(F)ccc1-c1cccc(-c2nc3cc(CNC[C@H]4CCCO4)cc(C(F)(F)F)c3o2)c1] |
| 157 | EV-9 | 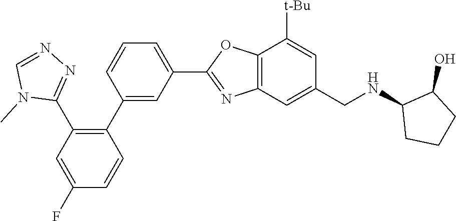 [SMILES: Cn1cnnc1-c1cc(F)ccc1-c1cccc(-c2nc3cc(CN[C@@H]4CCC[C@@H]4O)cc(C(C)(C)C)c3o2)c1] |
| 158 | EW-2 | 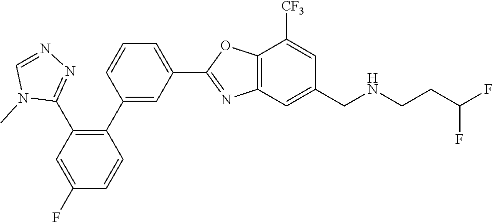 [SMILES: Cn1cnnc1-c1cc(F)ccc1-c1cccc(-c2nc3cc(CNCCC(F)F)cc(C(F)(F)F)c3o2)c1] |
| 159 | EX-6 | 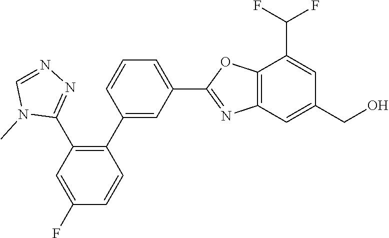 [SMILES: Cn1cnnc1-c1cc(F)ccc1-c1cccc(-c2nc3cc(CO)cc(C(F)F)c3o2)c1] |
| 160 | EY-2 | 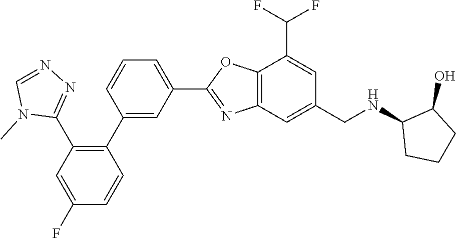 [SMILES: Cn1cnnc1-c1cc(F)ccc1-c1cccc(-c2nc3cc(CN[C@@H]4CCC[C@@H]4O)cc(C(F)F)c3o2)c1] |
| 161 | EZ-3 | 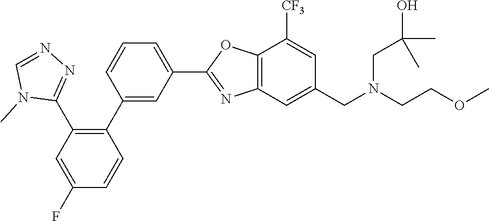 |
| 162 | FA-1 | 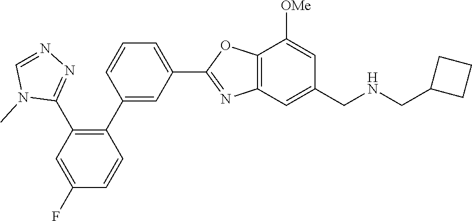 [SMILES: COc1cc(CNCC2CCC2)cc2nc(-c3cccc(-c4ccc(F)cc4-c4nncn4C)c3)oc12] |
| 163 | FB-2 | 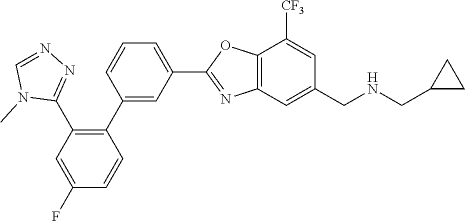 [SMILES: Cn1cnnc1-c1cc(F)ccc1-c1cccc(-c2nc3cc(CNCC4CC4)cc(C(F)(F)F)c3o2)c1] |
| 164 | FC-2 | 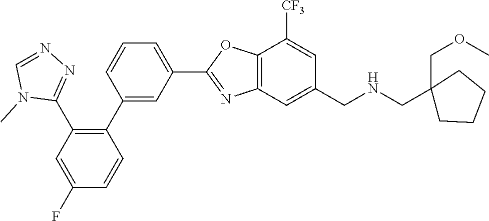 |
| 165 | FD-2 | 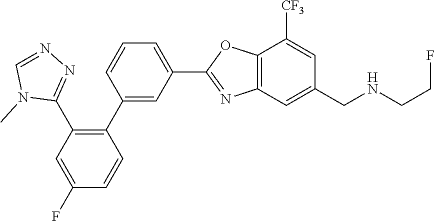 [SMILES: Cn1cnnc1-c1cc(F)ccc1-c1cccc(-c2nc3cc(CNCCF)cc(C(F)(F)F)c3o2)c1] |
| 166 | FE-3 | 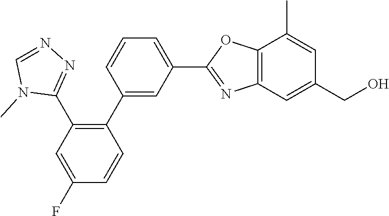 [SMILES: Cc1cc(CO)cc2nc(-c3cccc(-c4ccc(F)cc4-c4nncn4C)c3)oc12] |
| 167 | FF-6 | 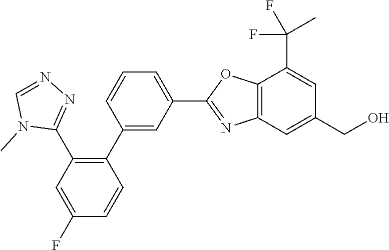 [SMILES: Cn1cnnc1-c1cc(F)ccc1-c1cccc(-c2nc3cc(CO)cc(C(C)(F)F)c3o2)c1] |
| 168 | FG-2 | 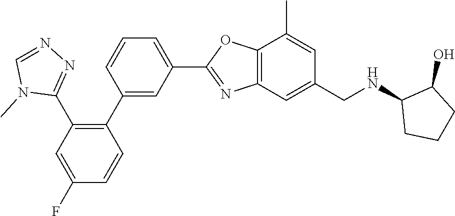 [SMILES: Cc1cc(CN[C@@H]2CCC[C@@H]2O)cc2nc(-c3cccc(-c4ccc(F)cc4-c4nncn4C)c3)oc12] |
| 169 | FH-2 | 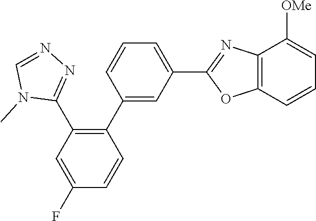 [SMILES: COc1cccc2oc(-c3cccc(-c4ccc(F)cc4-c4nncn4C)c3)nc12] |
| 170 | FI-2 | 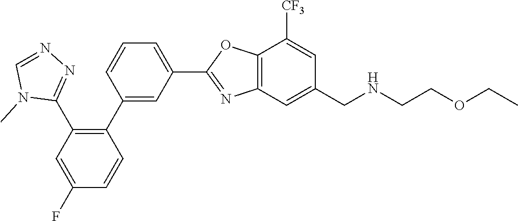 [SMILES: CCOCCNCc1cc(C(F)(F)F)c2oc(-c3cccc(-c4ccc(F)cc4-c4nncn4C)c3)nc2c1] |
| 171 | FJ-2 | 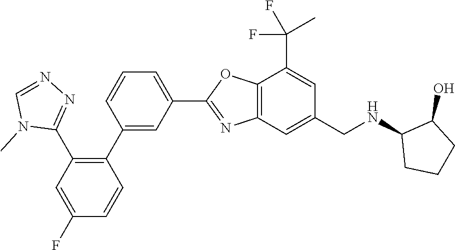 [SMILES: Cn1cnnc1-c1cc(F)ccc1-c1cccc(-c2nc3cc(CN[C@@H]4CCC[C@@H]4O)cc(C(C)(F)F)c3o2)c1] |
| 172 | FK-2 | 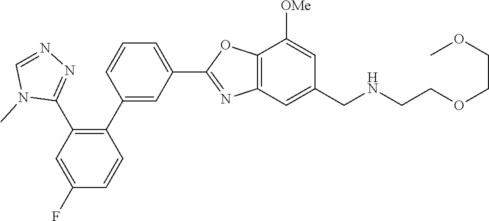 [SMILES: COCCOCCNCc1cc(OC)c2oc(-c3cccc(-c4ccc(F)cc4-c4nncn4C)c3)nc2c1] |
| 173 | FL-2 | 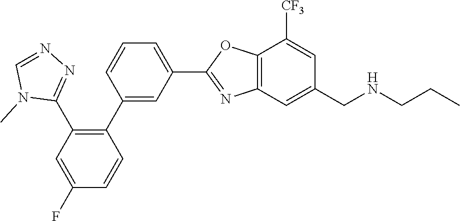 [SMILES: CCCNCc1cc(C(F)(F)F)c2oc(-c3cccc(-c4ccc(F)cc4-c4nncn4C)c3)nc2c1] |
| 174 | FM-2 | 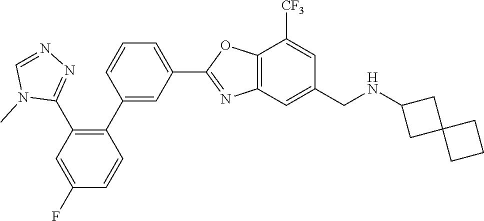 [SMILES: Cn1cnnc1-c1cc(F)ccc1-c1cccc(-c2nc3cc(CNC4CC5(CCC5)C4)cc(C(F)(F)F)c3o2)c1] |
| 175 | FN-1 | 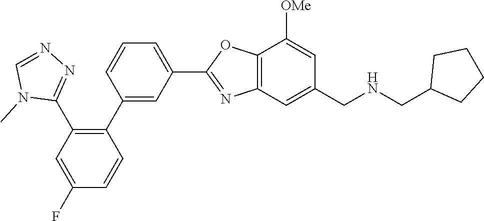 [SMILES: COc1cc(CNCC2CCCC2)cc2nc(-c3cccc(-c4ccc(F)cc4-c4nncn4C)c3)oc12] |
| 176 | FO-2 | 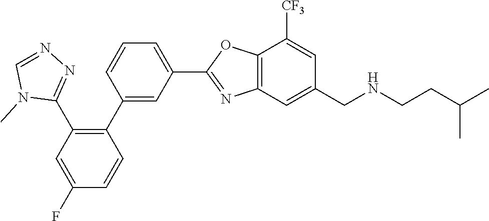 [SMILES: CC(C)CCNCc1cc(C(F)(F)F)c2oc(-c3cccc(-c4ccc(F)cc4-c4nncn4C)c3)nc2c1] |
| 177 | FP-1 | 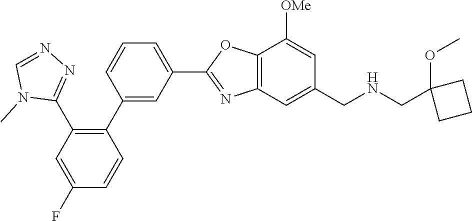 [SMILES: COc1cc(CNCC2(OC)CCC2)cc2nc(-c3cccc(-c4ccc(F)cc4-c4nncn4C)c3)oc12] |
| 178 | FQ-1 | 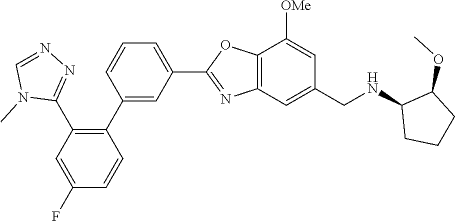 [SMILES: COc1cc(CN[C@@H]2CCC[C@@H]2OC)cc2nc(-c3cccc(-c4ccc(F)cc4-c4nncn4C)c3)oc12] |
| 179 | FR-1 | 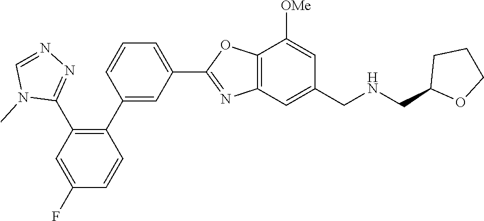 [SMILES: COc1cc(CNC[C@H]2CCCO2)cc2nc(-c3cccc(-c4ccc(F)cc4-c4nncn4C)c3)oc12] |
| 180 | FS-2 | 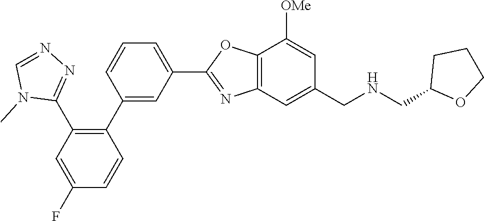 [SMILES: COc1cc(CNC[C@@H]2CCCO2)cc2nc(-c3cccc(-c4ccc(F)cc4-c4nncn4C)c3)oc12] |
| 181 | FT-1 | 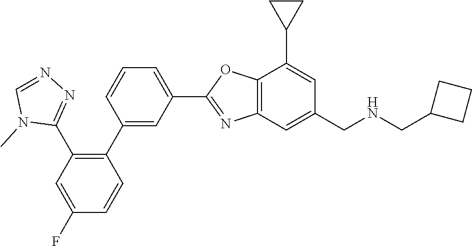 [SMILES: Cn1cnnc1-c1cc(F)ccc1-c1cccc(-c2nc3cc(CNCC4CCC4)cc(C4CC4)c3o2)c1] |
| 182 | FU-1 | 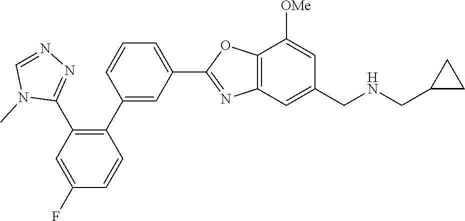 [SMILES: COc1cc(CNCC2CC2)cc2nc(-c3cccc(-c4ccc(F)cc4-c4nncn4C)c3)oc12] |
| 183 | FV-2 | 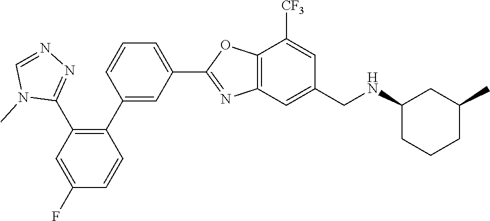 |
| 184 | FV-3 | 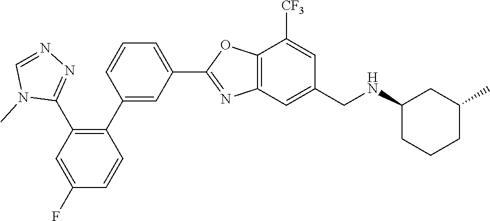 |
| 185 | FW-3 | 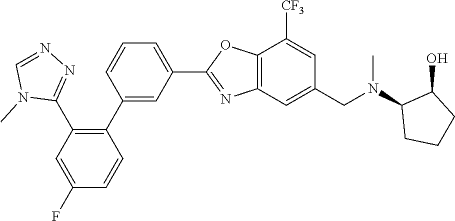 [SMILES: CN(Cc1cc(C(F)(F)F)c2oc(-c3cccc(-c4ccc(F)cc4-c4nncn4C)c3)nc2c1)[C@@H]1CCC[C@@H]1O] |
| 186 | FX-2 | 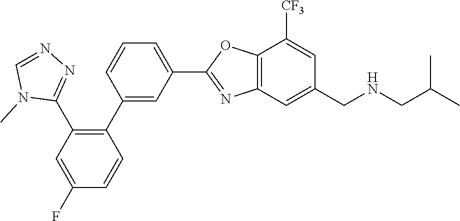 [SMILES: CC(C)CNCc1cc(C(F)(F)F)c2oc(-c3cccc(-c4ccc(F)cc4-c4nncn4C)c3)nc2c1] |
| 187 | FY-1 | 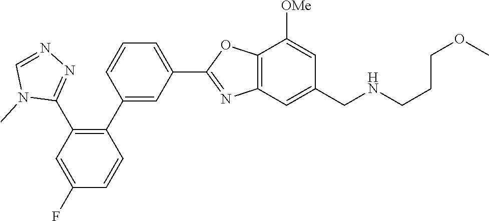 |
| 188 | FZ-5 | 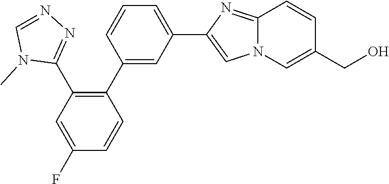 [SMILES: Cn1cnnc1-c1cc(F)ccc1-c1cccc(-c2cn3cc(CO)ccc3n2)c1] |
| 189 | GA-1 | 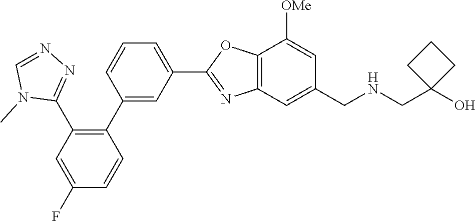 [SMILES: COc1cc(CNCC2(O)CCC2)cc2nc(-c3cccc(-c4ccc(F)cc4-c4nncn4C)c3)oc12] |
| 190 | GB-2 | 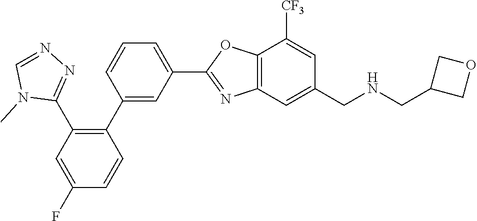 [SMILES: Cn1cnnc1-c1cc(F)ccc1-c1cccc(-c2nc3cc(CNCC4COC4)cc(C(F)(F)F)c3o2)c1] |
| 191 | GC-2 | 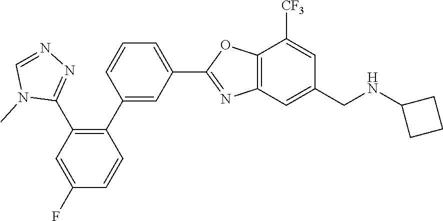 [SMILES: Cn1cnnc1-c1cc(F)ccc1-c1cccc(-c2nc3cc(CNC4CCC4)cc(C(F)(F)F)c3o2)c1] |
| 192 | GD-1 | 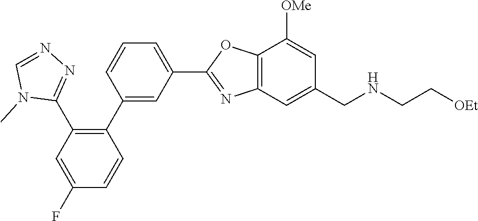 [SMILES: CCOCCNCc1cc(OC)c2oc(-c3cccc(-c4ccc(F)cc4-c4nncn4C)c3)nc2c1] |
| 193 | GE-2 | 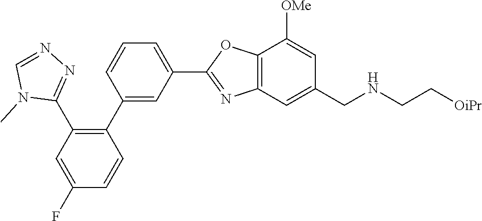 [SMILES: COc1cc(CNCCOC(C)C)cc2nc(-c3cccc(-c4ccc(F)cc4-c4nncn4C)c3)oc12] |
| 194 | GF-2 | 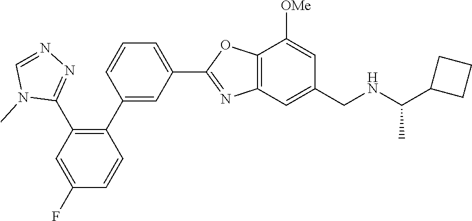 [SMILES: COc1cc(CN[C@@H](C)C2CCC2)cc2nc(-c3cccc(-c4ccc(F)cc4-c4nncn4C)c3)oc12] |
| 195 | GG-2 | 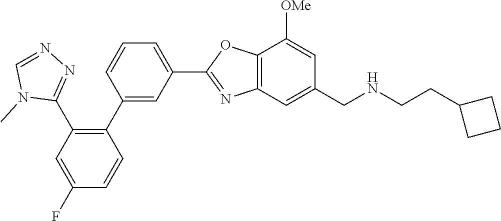 [SMILES: COc1cc(CNCCC2CCC2)cc2nc(-c3cccc(-c4ccc(F)cc4-c4nncn4C)c3)oc12] |
| 196 | GH-1 | 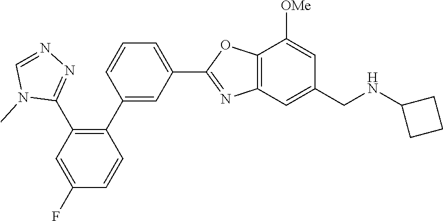 [SMILES: COc1cc(CNC2CCC2)cc2nc(-c3cccc(-c4ccc(F)cc4-c4nncn4C)c3)oc12] |
| 197 | GI-2 | 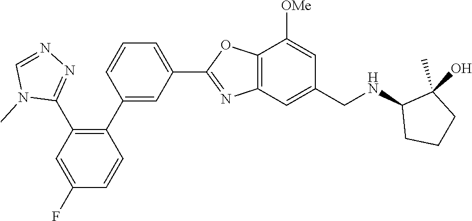 [SMILES: COc1cc(CN[C@@H]2CCC[C@]2(C)O)cc2nc(-c3cccc(-c4ccc(F)cc4-c4nncn4C)c3)oc12] |
| 198 | GJ-2 | 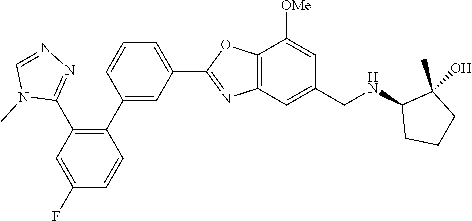 [SMILES: COc1cc(CN[C@@H]2CCC[C@@]2(C)O)cc2nc(-c3cccc(-c4ccc(F)cc4-c4nncn4C)c3)oc12] |
| 199 | GK-5 | 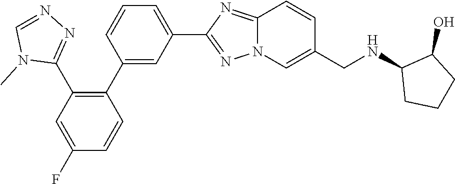 [SMILES: Cn1cnnc1-c1cc(F)ccc1-c1cccc(-c2nc3ccc(CN[C@@H]4CCC[C@@H]4O)cn3n2)c1] |
| 200 | GL-2 | 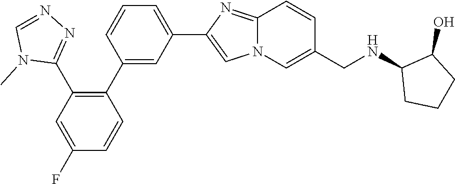 [SMILES: Cn1cnnc1-c1cc(F)ccc1-c1cccc(-c2cn3cc(CN[C@@H]4CCC[C@@H]4O)ccc3n2)c1] |
| 201 | GM-2 | 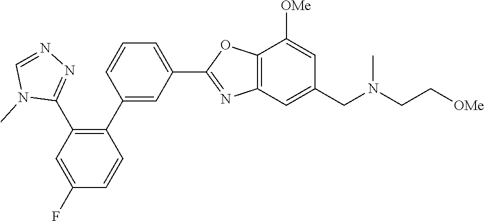 [SMILES: COCCN(C)Cc1cc(OC)c2oc(-c3cccc(-c4ccc(F)cc4-c4nncn4C)c3)nc2c1] |
| 202 | GN-5 | 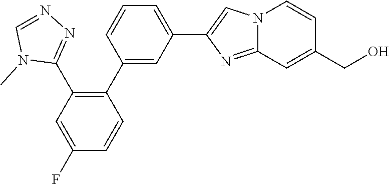 [SMILES: Cn1cnnc1-c1cc(F)ccc1-c1cccc(-c2cn3ccc(CO)cc3n2)c1] |
| 203 | GO-2 | 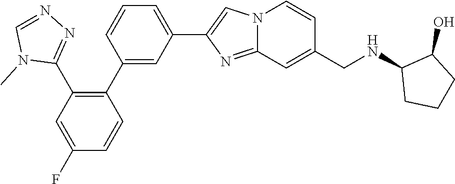 [SMILES: Cn1cnnc1-c1cc(F)ccc1-c1cccc(-c2cn3ccc(CN[C@@H]4CCC[C@@H]4O)cc3n2)c1] |
| 204 | GP-2 | 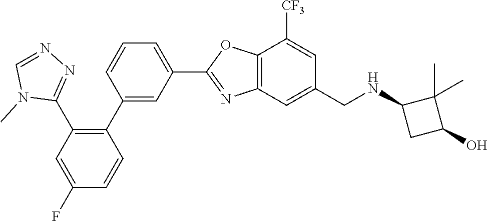 [SMILES: Cn1cnnc1-c1cc(F)ccc1-c1cccc(-c2nc3cc(CN[C@@H]4C[C@H](O)C4(C)C)cc(C(F)(F)F)c3o2)c1] |
| 205 | GQ-6 | 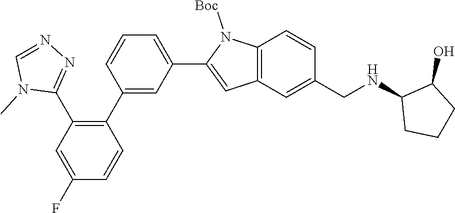 [SMILES: Cn1cnnc1-c1cc(F)ccc1-c1cccc(-c2cc3cc(CN[C@@H]4CCC[C@@H]4O)ccc3n2C(=O)OC(C)(C)C)c1] |
| 206 | GR-2 | 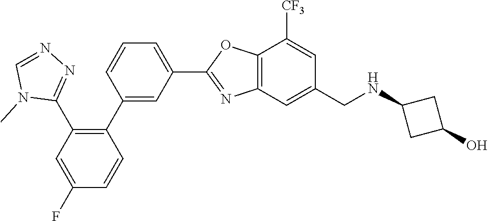 [SMILES: Cn1cnnc1-c1cc(F)ccc1-c1cccc(-c2nc3cc(CN[C@H]4C[C@@H](O)C4)cc(C(F)(F)F)c3o2)c1] |
| 207 | GS-2 | 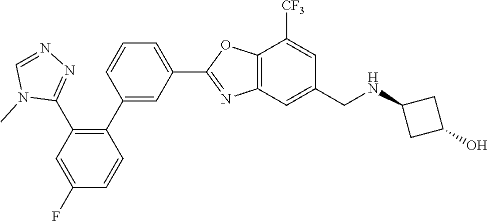 [SMILES: Cn1cnnc1-c1cc(F)ccc1-c1cccc(-c2nc3cc(CN[C@H]4C[C@H](O)C4)cc(C(F)(F)F)c3o2)c1] |
| 208 | GT-1 | 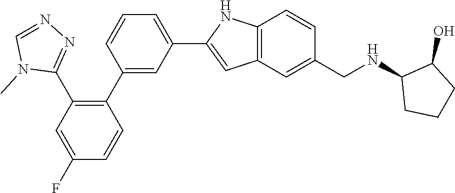 [SMILES: Cn1cnnc1-c1cc(F)ccc1-c1cccc(-c2cc3cc(CN[C@@H]4CCC[C@@H]4O)ccc3[nH]2)c1] |
| 209 | GU-1 | 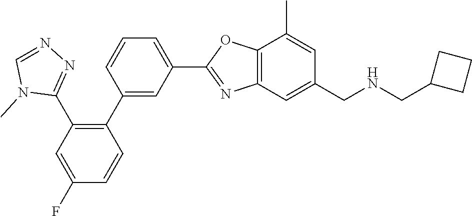 [SMILES: Cc1cc(CNCC2CCC2)cc2nc(-c3cccc(-c4ccc(F)cc4-c4nncn4C)c3)oc12] |
| 210 | GV-1 | 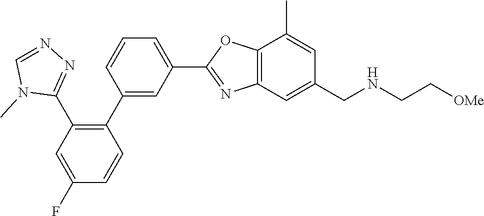 [SMILES: COCCNCc1cc(C)c2oc(-c3cccc(-c4ccc(F)cc4-c4nncn4C)c3)nc2c1] |
| 211 | GW-2 | 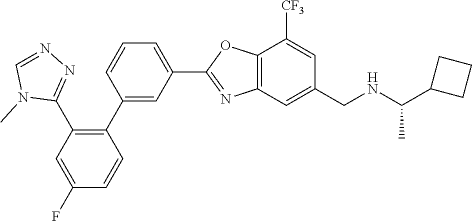 [SMILES: C[C@H](NCc1cc(C(F)(F)F)c2oc(-c3cccc(-c4ccc(F)cc4-c4nncn4C)c3)nc2c1)C1CCC1] |
| 212 | GX-1 | 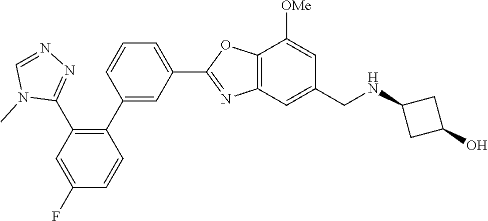 [SMILES: COc1cc(CN[C@H]2C[C@@H](O)C2)cc2nc(-c3cccc(-c4ccc(F)cc4-c4nncn4C)c3)oc12] |
| 213 | GY-1 | 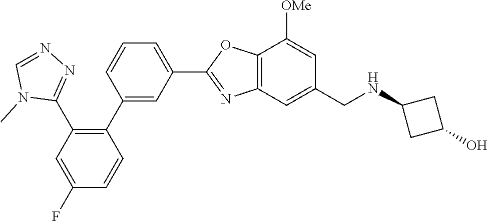 [SMILES: COc1cc(CN[C@H]2C[C@H](O)C2)cc2nc(-c3cccc(-c4ccc(F)cc4-c4nncn4C)c3)oc12] |
| 214 | GZ-1 | 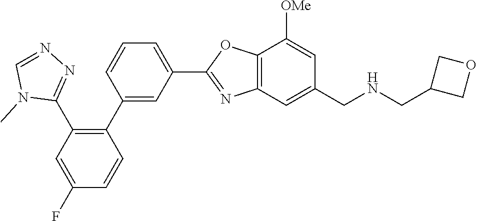 [SMILES: COc1cc(CNCC2COC2)cc2nc(-c3cccc(-c4ccc(F)cc4-c4nncn4C)c3)oc12] |
| 215 | HA-3 | 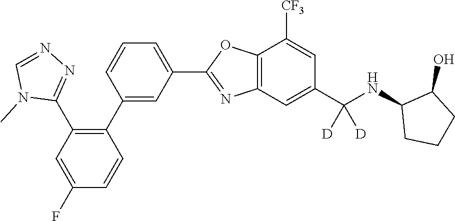 [SMILES: [2H]C([2H])(N[C@@H]1CCC[C@@H]1O)c1cc(C(F)(F)F)c2oc(-c3cccc(-c4ccc(F)cc4-c4nncn4C)c3)nc2c1] |
| 216 | HB-1 | 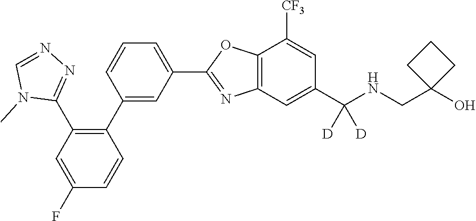 [SMILES: [2H]C([2H])(NCC1(O)CCC1)c1cc(C(F)(F)F)c2oc(-c3cccc(-c4ccc(F)cc4-c4nncn4C)c3)nc2c1] |
| 217 | HC-2 | 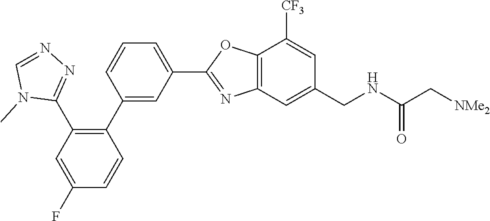 [SMILES: CN(C)CC(=O)NCc1cc(C(F)(F)F)c2oc(-c3cccc(-c4ccc(F)cc4-c4nncn4C)c3)nc2c1] |
| 218 | HD-2 | 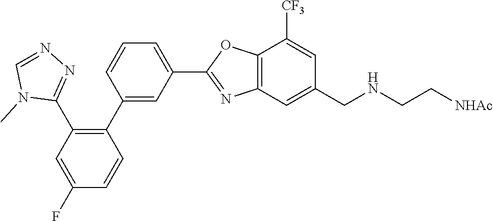 [SMILES: CC(=O)NCCNCc1cc(C(F)(F)F)c2oc(-c3cccc(-c4ccc(F)cc4-c4nncn4C)c3)nc2c1] |
| 219 | HE-4 | 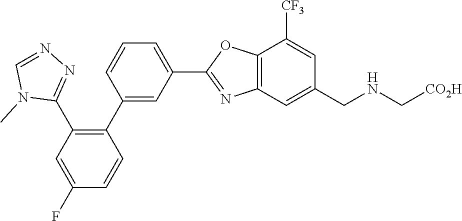 [SMILES: Cn1cnnc1-c1cc(F)ccc1-c1cccc(-c2nc3cc(CNCC(=O)O)cc(C(F)(F)F)c3o2)c1] |
| 220 | HF-1 | 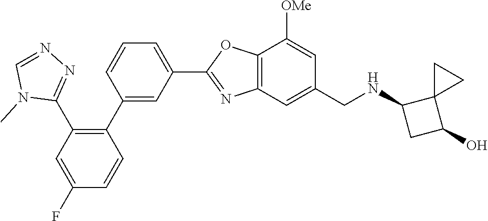 [SMILES: COc1cc(CN[C@@H]2C[C@H](O)C23CC3)cc2nc(-c3cccc(-c4ccc(F)cc4-c4nncn4C)c3)oc12] |
| 221 | AAB-9 | 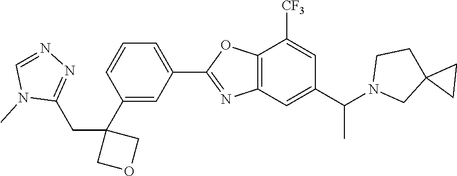 [SMILES: CC(c1cc(C(F)(F)F)c2oc(-c3cccc(C4(Cc5nncn5C)COC4)c3)nc2c1)N1CCC2(CC2)C1] |
| 222 | AAC-5 | 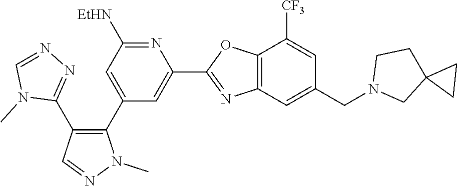 |
| 223 | AAD-1 | 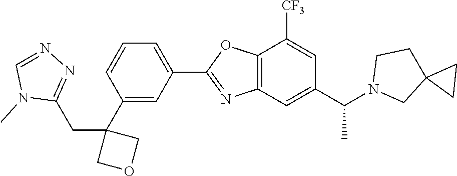 [SMILES: C[C@H](c1cc(C(F)(F)F)c2oc(-c3cccc(C4(Cc5nncn5C)COC4)c3)nc2c1)N1CCC2(CC2)C1] |
| 224 | AAE-1 | 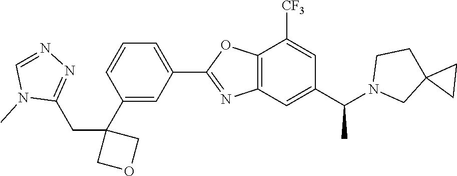 [SMILES: C[C@@H](c1cc(C(F)(F)F)c2oc(-c3cccc(C4(Cc5nncn5C)COC4)c3)nc2c1)N1CCC2(CC2)C1] |
| 225 | AAG-4 | 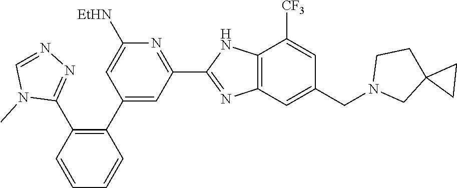 |
| 226 | AAI-5 | 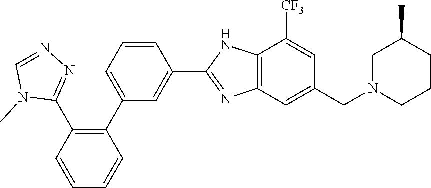 [SMILES: C[C@H]1CCCN(Cc2cc(C(F)(F)F)c3[nH]c(-c4cccc(-c5ccccc5-c5nncn5C)c4)nc3c2)C1] |
| 227 | AAJ-1 | 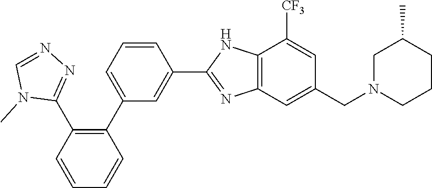 [SMILES: C[C@@H]1CCCN(Cc2cc(C(F)(F)F)c3[nH]c(-c4cccc(-c5ccccc5-c5nncn5C)c4)nc3c2)C1] |
| 228 | AAK-1 | 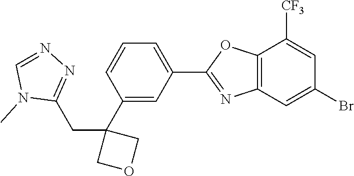 [SMILES: Cn1cnnc1CC1(c2cccc(-c3nc4cc(Br)cc(C(F)(F)F)c4o3)c2)COC1] |
| 229 | AAL-5 | 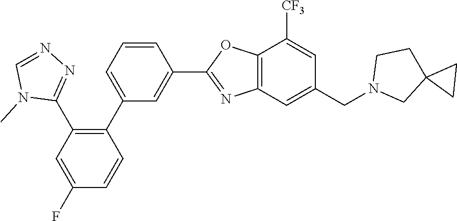 [SMILES: Cn1cnnc1-c1cc(F)ccc1-c1cccc(-c2nc3cc(CN4CCC5(CC5)C4)cc(C(F)(F)F)c3o2)c1] |
| 230 | AAM-2 | 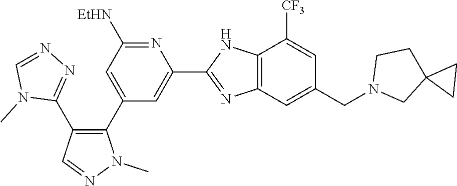 |
| 231 | AAN-3 | 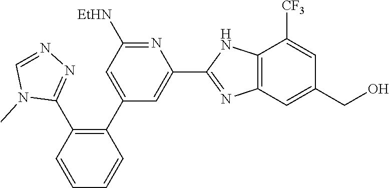 |
| 232 | AAO-4 | 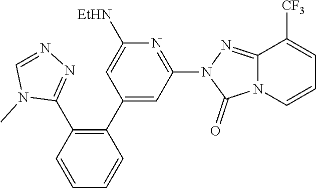 |
| 233 | AAP-7 | 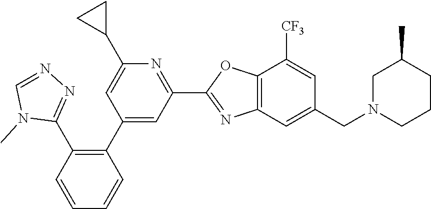 [SMILES: C[C@H]1CCCN(Cc2cc(C(F)(F)F)c3oc(-c4cc(-c5ccccc5-c5nncn5C)cc(C5CC5)n4)nc3c2)C1] |
| 234 | AAQ-2 | 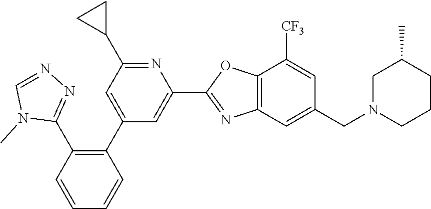 [SMILES: C[C@@H]1CCCN(Cc2cc(C(F)(F)F)c3oc(-c4cc(-c5ccccc5-c5nncn5C)cc(C5CC5)n4)nc3c2)C1] |
| 235 | AAR-2 | 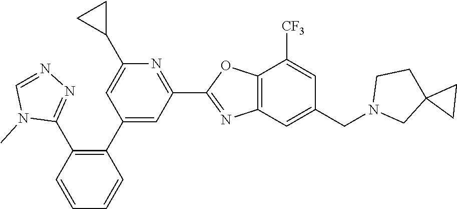 [SMILES: Cn1cnnc1-c1ccccc1-c1cc(-c2nc3cc(CN4CCC5(CC5)C4)cc(C(F)(F)F)c3o2)nc(C2CC2)c1] |
| 236 | AAS-2 | 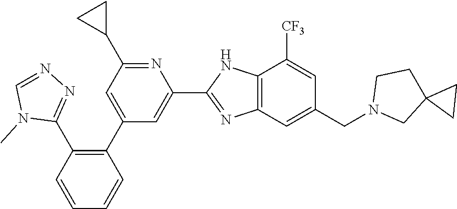 [SMILES: Cn1cnnc1-c1ccccc1-c1cc(-c2nc3cc(CN4CCC5(CC5)C4)cc(C(F)(F)F)c3[nH]2)nc(C2CC2)c1] |
| 237 | AAT-14 | 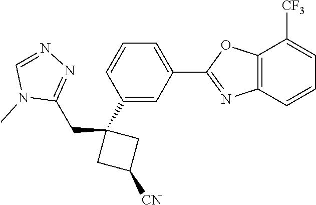 [SMILES: Cn1cnnc1C[C@]1(c2cccc(-c3nc4cccc(C(F)(F)F)c4o3)c2)C[C@H](C#N)C1] |
| 238 | AAU-6 | 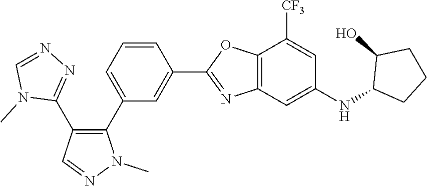 [SMILES: Cn1cnnc1-c1cnn(C)c1-c1cccc(-c2nc3cc(N[C@H]4CCC[C@@H]4O)cc(C(F)(F)F)c3o2)c1] |
| 239 | AAV-2 | 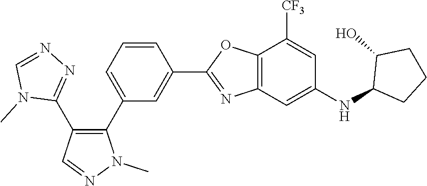 [SMILES: Cn1cnnc1-c1cnn(C)c1-c1cccc(-c2nc3cc(N[C@@H]4CCC[C@H]4O)cc(C(F)(F)F)c3o2)c1] |
| 240 | AAW-5 | 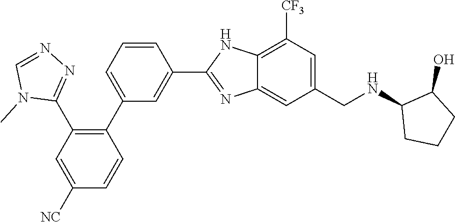 [SMILES: Cn1cnnc1-c1cc(C#N)ccc1-c1cccc(-c2nc3cc(CN[C@@H]4CCC[C@@H]4O)cc(C(F)(F)F)c3[nH]2)c1] |
| 241 | AAX-12 | 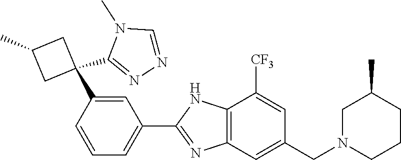 [SMILES: C[C@H]1CCCN(Cc2cc(C(F)(F)F)c3[nH]c(-c4cccc([C@]5(c6nncn6C)C[C@@H](C)C5)c4)nc3c2)C1] |
| 242 | AAY-2 | 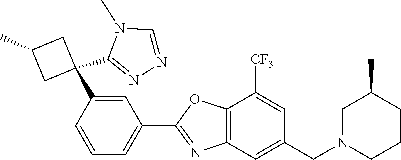 [SMILES: C[C@H]1CCCN(Cc2cc(C(F)(F)F)c3oc(-c4cccc([C@]5(c6nncn6C)C[C@@H](C)C5)c4)nc3c2)C1] |
| 243 | AAZ-10 | 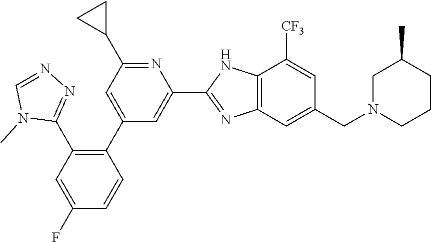 [SMILES: C[C@H]1CCCN(Cc2cc(C(F)(F)F)c3[nH]c(-c4cc(-c5ccc(F)cc5-c5nncn5C)cc(C5CC5)n4)nc3c2)C1] |
| 244 | ABA-2 | 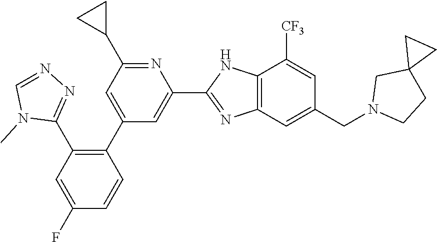 [SMILES: Cn1cnnc1-c1cc(F)ccc1-c1cc(-c2nc3cc(CN4CCC5(CC5)C4)cc(C(F)(F)F)c3[nH]2)nc(C2CC2)c1] |
| 245 | ABB-2 | 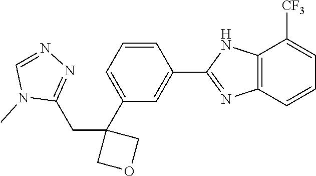 [SMILES: Cn1cnnc1CC1(c2cccc(-c3nc4cccc(C(F)(F)F)c4[nH]3)c2)COC1] |
| 246 | ABC-4 | 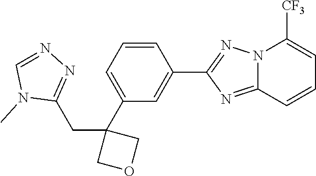 [SMILES: Cn1cnnc1CC1(c2cccc(-c3nc4cccc(C(F)(F)F)n4n3)c2)COC1] |
| 247 | ABD-9 | 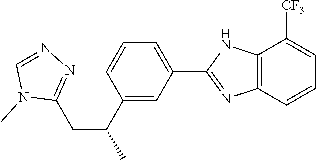 [SMILES: C[C@H](Cc1nncn1C)c1cccc(-c2nc3cccc(C(F)(F)F)c3[nH]2)c1] |
| 248 | ABE-2 | 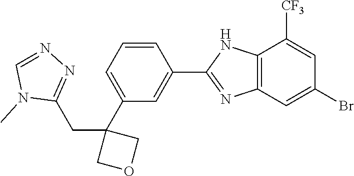 [SMILES: Cn1cnnc1CC1(c2cccc(-c3nc4cc(Br)cc(C(F)(F)F)c4[nH]3)c2)COC1] |
| 249 | ABF-4 | 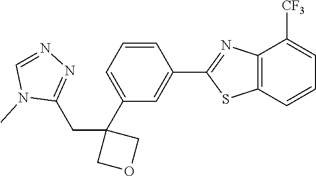 [SMILES: Cn1cnnc1CC1(c2cccc(-c3nc4c(C(F)(F)F)cccc4s3)c2)COC1] |
| 250 | ABG-3 | 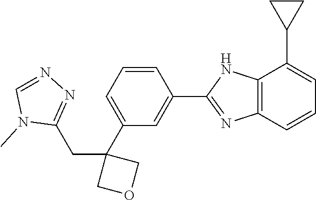 [SMILES: Cn1cnnc1CC1(c2cccc(-c3nc4cccc(C5CC5)c4[nH]3)c2)COC1] |
| 251 | ABH-4 | 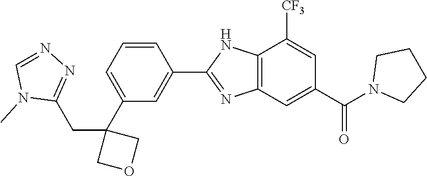 [SMILES: Cn1cnnc1CC1(c2cccc(-c3nc4cc(C(=O)N5CCCC5)cc(C(F)(F)F)c4[nH]3)c2)COC1] |
| 252 | ABI-7 | 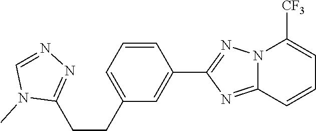 [SMILES: Cn1cnnc1CCc1cccc(-c2nc3cccc(C(F)(F)F)n3n2)c1] |
| 253 | ABJ-2 | 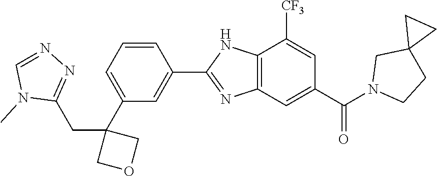 [SMILES: Cn1cnnc1CC1(c2cccc(-c3nc4cc(C(=O)N5CCC6(CC6)C5)cc(C(F)(F)F)c4[nH]3)c2)COC1] |
| 254 | ABK-1 | 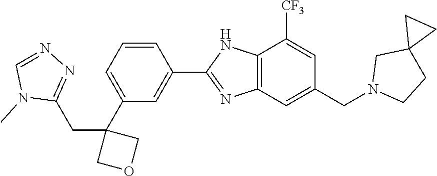 [SMILES: Cn1cnnc1CC1(c2cccc(-c3nc4cc(CN5CCC6(CC6)C5)cc(C(F)(F)F)c4[nH]3)c2)COC1] |
| 255 | ABL-1 | 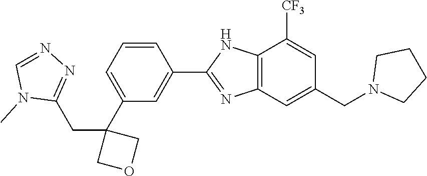 [SMILES: Cn1cnnc1CC1(c2cccc(-c3nc4cc(CN5CCCC5)cc(C(F)(F)F)c4[nH]3)c2)COC1] |
| 256 | ABM-2 | 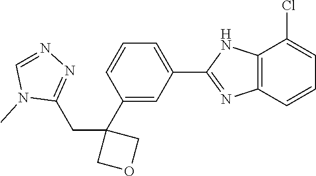 [SMILES: Cn1cnnc1CC1(c2cccc(-c3nc4cccc(Cl)c4[nH]3)c2)COC1] |
| 257 | ABN-2 | 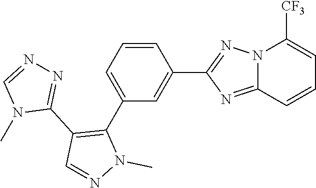 [SMILES: Cn1cnnc1-c1cnn(C)c1-c1cccc(-c2nc3cccc(C(F)(F)F)n3n2)c1] |
| 258 | ABO-1 | 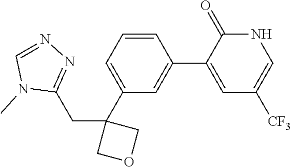 [SMILES: Cn1cnnc1CC1(c2cccc(-c3cc(C(F)(F)F)c[nH]c3=O)c2)COC1] |
| 259 | ABP-2 | 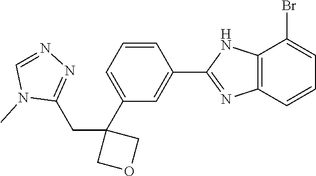 [SMILES: Cn1cnnc1CC1(c2cccc(-c3nc4cccc(Br)c4[nH]3)c2)COC1] |
| 260 | ABQ-2 | 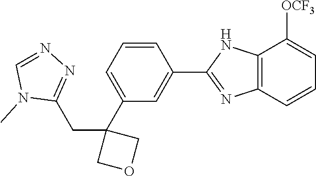 [SMILES: Cn1cnnc1CC1(c2cccc(-c3nc4cccc(OC(F)(F)F)c4[nH]3)c2)COC1] |
| 261 | ABR-2 | 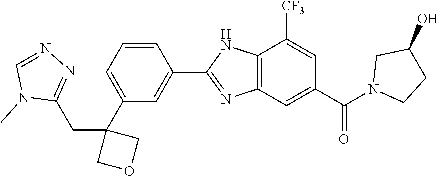 [SMILES: Cn1cnnc1CC1(c2cccc(-c3nc4cc(C(=O)N5CC[C@H](O)C5)cc(C(F)(F)F)c4[nH]3)c2)COC1] |
| 262 | ABS-2 | 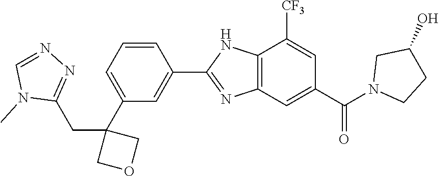 [SMILES: Cn1cnnc1CC1(c2cccc(-c3nc4cc(C(=O)N5CC[C@@H](O)C5)cc(C(F)(F)F)c4[nH]3)c2)COC1] |
| 263 | ABT-2 | 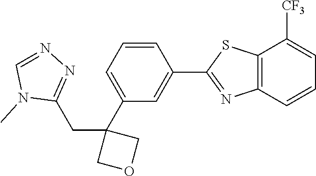 [SMILES: Cn1cnnc1CC1(c2cccc(-c3nc4cccc(C(F)(F)F)c4s3)c2)COC1] |
| 264 | ABU-2 | 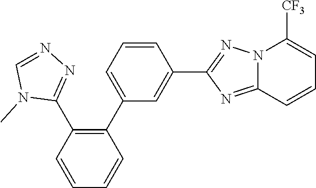 [SMILES: Cn1cnnc1-c1ccccc1-c1cccc(-c2nc3cccc(C(F)(F)F)n3n2)c1] |
| 265 | ABV-1 | 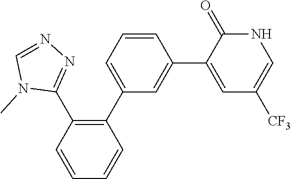 [SMILES: Cn1cnnc1-c1ccccc1-c1cccc(-c2cc(C(F)(F)F)c[nH]c2=O)c1] |
| 266 | ABW-1 | 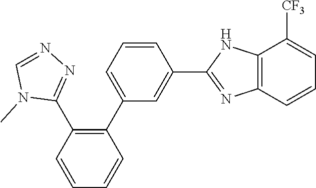 [SMILES: Cn1cnnc1-c1ccccc1-c1cccc(-c2nc3cccc(C(F)(F)F)c3[nH]2)c1] |
| 267 | ABX-3 | 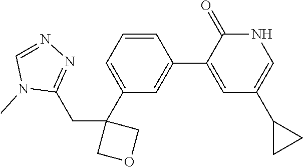 [SMILES: Cn1cnnc1CC1(c2cccc(-c3cc(C4CC4)c[nH]c3=O)c2)COC1] |
| 268 | ABY-1 | 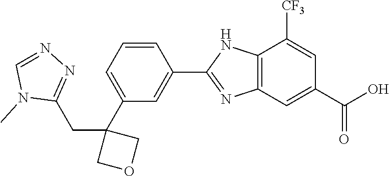 [SMILES: Cn1cnnc1CC1(c2cccc(-c3nc4cc(C(=O)O)cc(C(F)(F)F)c4[nH]3)c2)COC1] |
| 269 | ABZ-2 | 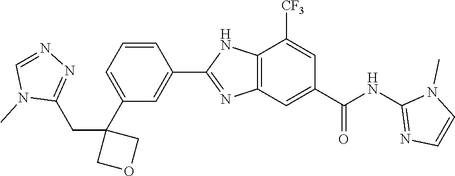 [SMILES: Cn1cnnc1CC1(c2cccc(-c3nc4cc(C(=O)Nc5nccn5C)cc(C(F)(F)F)c4[nH]3)c2)COC1] |
| 270 | ACA-2 | 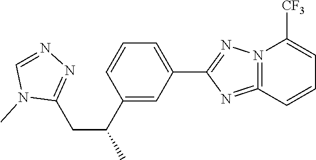 |
| 271 | ACB-1 | 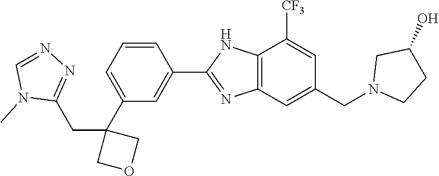 [SMILES: Cn1cnnc1CC1(c2cccc(-c3nc4cc(CN5CC[C@@H](O)C5)cc(C(F)(F)F)c4[nH]3)c2)COC1] |
| 272 | ACC-1 | 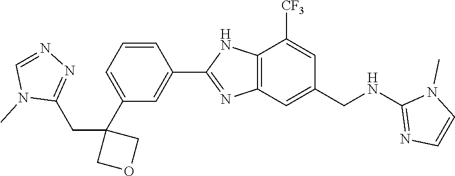 [SMILES: Cn1cnnc1CC1(c2cccc(-c3nc4cc(CNc5nccn5C)cc(C(F)(F)F)c4[nH]3)c2)COC1] |
| 273 | ACD-1 | 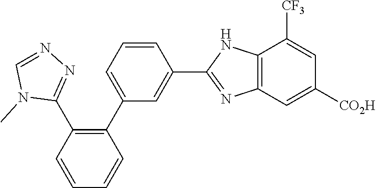 [SMILES: Cn1cnnc1-c1ccccc1-c1cccc(-c2nc3cc(C(=O)O)cc(C(F)(F)F)c3[nH]2)c1] |
| 274 | ACE-1 | 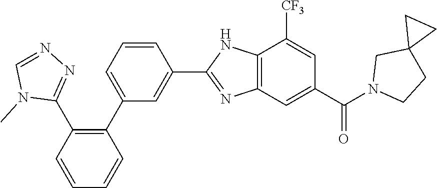 [SMILES: Cn1cnnc1-c1ccccc1-c1cccc(-c2nc3cc(C(=O)N4CCC5(CC5)C4)cc(C(F)(F)F)c3[nH]2)c1] |
| 275 | ACF-1 | 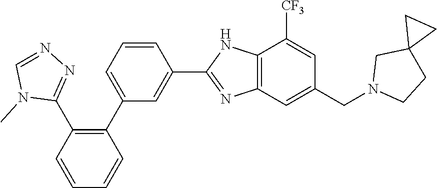 [SMILES: Cn1cnnc1-c1ccccc1-c1cccc(-c2nc3cc(CN4CCC5(CC5)C4)cc(C(F)(F)F)c3[nH]2)c1] |
| 276 | ACG-3 | 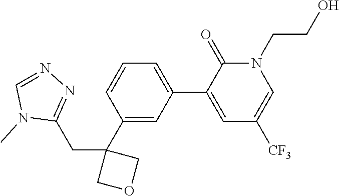 [SMILES: Cn1cnnc1CC1(c2cccc(-c3cc(C(F)(F)F)cn(CCO)c3=O)c2)COC1] |
| 277 | ACH-5 | 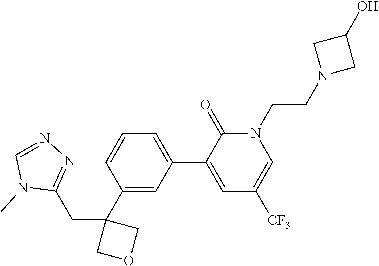 [SMILES: Cn1cnnc1CC1(c2cccc(-c3cc(C(F)(F)F)cn(CCN4CC(O)C4)c3=O)c2)COC1] |
| 278 | ACI-3 | 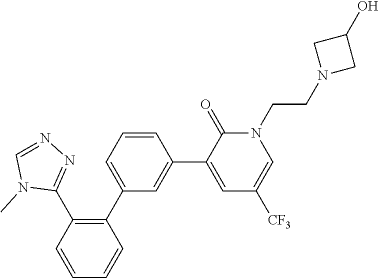 [SMILES: Cn1cnnc1-c1ccccc1-c1cccc(-c2cc(C(F)(F)F)cn(CCN3CC(O)C3)c2=O)c1] |
| 279 | ACJ-5 | 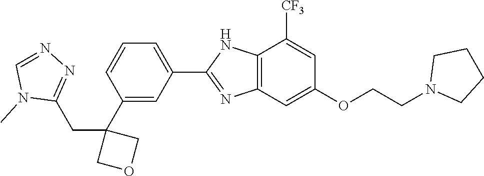 [SMILES: Cn1cnnc1CC1(c2cccc(-c3nc4cc(OCCN5CCCC5)cc(C(F)(F)F)c4[nH]3)c2)COC1] |
| 280 | ACK-3 | 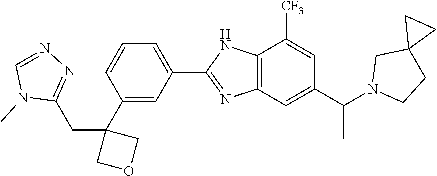 [SMILES: CC(c1cc(C(F)(F)F)c2[nH]c(-c3cccc(C4(Cc5nncn5C)COC4)c3)nc2c1)N1CCC2(CC2)C1] |
| 281 | ACL-1 | 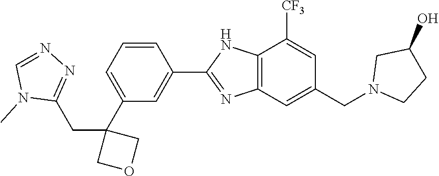 [SMILES: Cn1cnnc1CC1(c2cccc(-c3nc4cc(CN5CC[C@H](O)C5)cc(C(F)(F)F)c4[nH]3)c2)COC1] |
| 282 | ACM-1 | 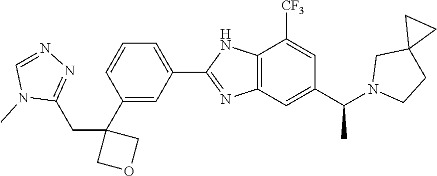 [SMILES: C[C@@H](c1cc(C(F)(F)F)c2[nH]c(-c3cccc(C4(Cc5nncn5C)COC4)c3)nc2c1)N1CCC2(CC2)C1] |
| 283 | ACN-1 | 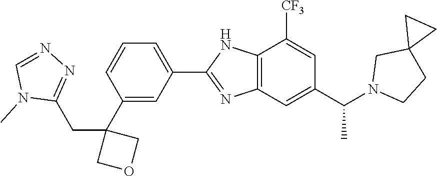 [SMILES: C[C@H](c1cc(C(F)(F)F)c2[nH]c(-c3cccc(C4(Cc5nncn5C)COC4)c3)nc2c1)N1CCC2(CC2)C1] |
| 284 | ACO-1 | 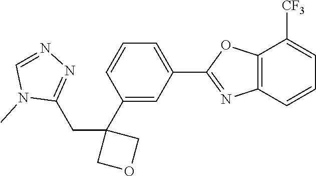 [SMILES: Cn1cnnc1CC1(c2cccc(-c3nc4cccc(C(F)(F)F)c4o3)c2)COC1] |
| 285 | ACP-4 | 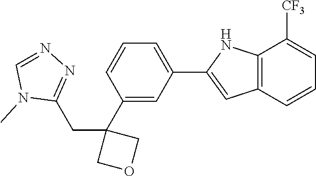 [SMILES: Cn1cnnc1CC1(c2cccc(-c3cc4cccc(C(F)(F)F)c4[nH]3)c2)COC1] |
| 286 | ACQ-3 | 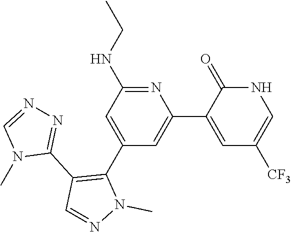 [SMILES: CCNc1cc(-c2c(-c3nncn3C)cnn2C)cc(-c2cc(C(F)(F)F)c[nH]c2=O)n1] |
| 287 | ACR-3 | 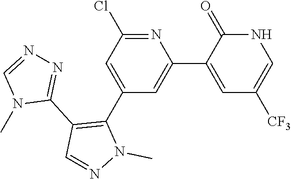 [SMILES: Cn1cnnc1-c1cnn(C)c1-c1cc(Cl)nc(-c2cc(C(F)(F)F)c[nH]c2=O)c1] |
| 288 | ACS-3 | 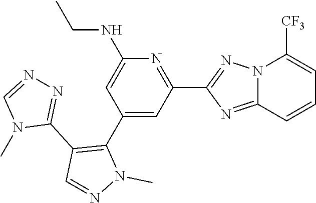 [SMILES: CCNc1cc(-c2c(-c3nncn3C)cnn2C)cc(-c2nc3cccc(C(F)(F)F)n3n2)n1] |
| 289 | ACT-3 | 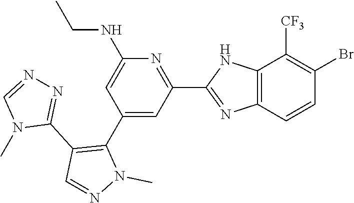 [SMILES: CCNc1cc(-c2c(-c3nncn3C)cnn2C)cc(-c2nc3ccc(Br)c(C(F)(F)F)c3[nH]2)n1] |
| 290 | ACU-2 | 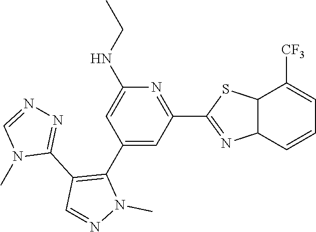 [SMILES: CCNc1cc(-c2c(-c3nncn3C)cnn2C)cc(C2=NC3C=CC=C(C(F)(F)F)C3S2)n1] |
| 291 | ACV-5 | 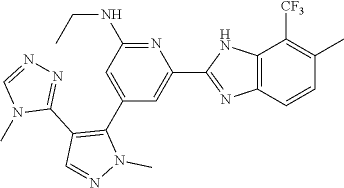 |
| 292 | ACW-2 | 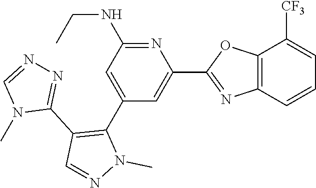 [SMILES: CCNc1cc(-c2c(-c3nncn3C)cnn2C)cc(-c2nc3cccc(C(F)(F)F)c3o2)n1] |
| 293 | ACZ-8 | 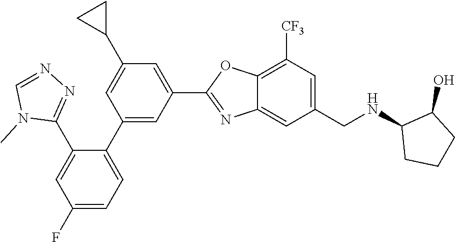 [SMILES: Cn1cnnc1-c1cc(F)ccc1-c1cc(-c2nc3cc(CN[C@@H]4CCC[C@@H]4O)cc(C(F)(F)F)c3o2)cc(C2CC2)c1] |
| 294 | ADA-6 | 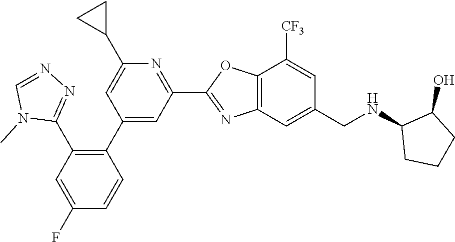 [SMILES: Cn1cnnc1-c1cc(F)ccc1-c1cc(-c2nc3cc(CN[C@@H]4CCC[C@@H]4O)cc(C(F)(F)F)c3o2)nc(C2CC2)c1] |
| 295 | ADB-9 | 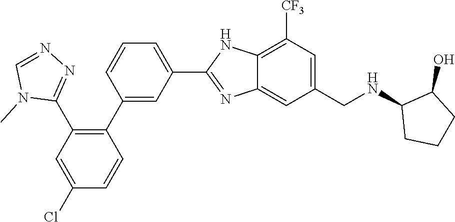 [SMILES: Cn1cnnc1-c1cc(Cl)ccc1-c1cccc(-c2nc3cc(CN[C@@H]4CCC[C@@H]4O)cc(C(F)(F)F)c3[nH]2)c1] |
| 296 | ADC-3 | 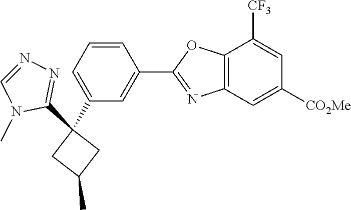 [SMILES: COC(=O)c1cc(C(F)(F)F)c2oc(-c3cccc([C@]4(c5nncn5C)C[C@@H](C)C4)c3)nc2c1] |
| 297 | ADD-1 | 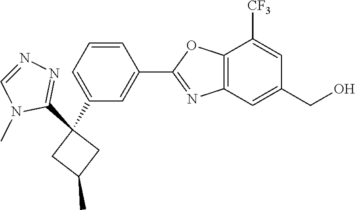 [SMILES: Cn1cnnc1[C@]1(c2cccc(-c3nc4cc(CO)cc(C(F)(F)F)c4o3)c2)C[C@H](C)C1] |
| 298 | ADE-2 | 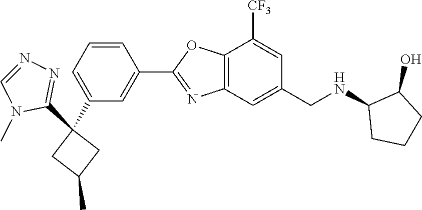 [SMILES: Cn1cnnc1[C@]1(c2cccc(-c3nc4cc(CN[C@@H]5CCC[C@@H]5O)cc(C(F)(F)F)c4o3)c2)C[C@H](C)C1] |
| 299 | ADF-2 | 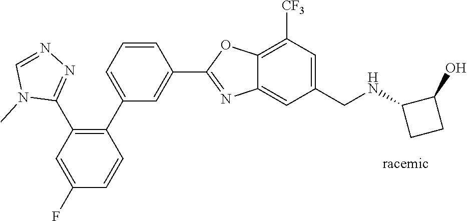 [SMILES: Cn1cnnc1-c1cc(F)ccc1-c1cccc(-c2nc3cc(CN[C@H]4CC[C@@H]4O)cc(C(F)(F)F)c3o2)c1] |
| 300 | ADG-1 | 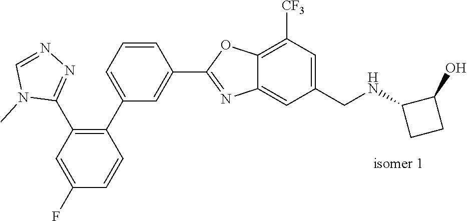 [SMILES: Cn1cnnc1-c1cc(F)ccc1-c1cccc(-c2nc3cc(CN[C@H]4CC[C@@H]4O)cc(C(F)(F)F)c3o2)c1] |
| 301 | ADH-1 | 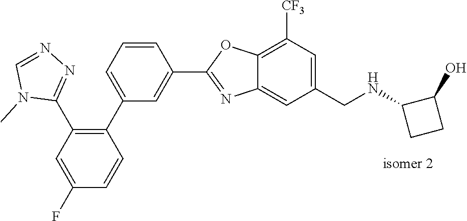 [SMILES: Cn1cnnc1-c1cc(F)ccc1-c1cccc(-c2nc3cc(CN[C@H]4CC[C@@H]4O)cc(C(F)(F)F)c3o2)c1] |
| 302 | ADI-3 | 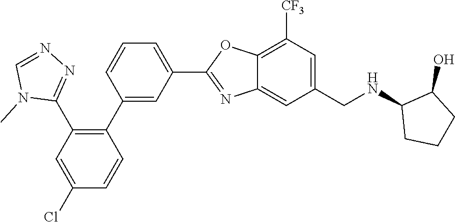 [SMILES: Cn1cnnc1-c1cc(Cl)ccc1-c1cccc(-c2nc3cc(CN[C@@H]4CCC[C@@H]4O)cc(C(F)(F)F)c3o2)c1] |
| 303 | ADJ-2 | 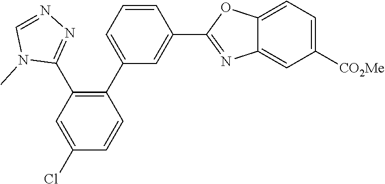 [SMILES: COC(=O)c1ccc2oc(-c3cccc(-c4ccc(Cl)cc4-c4nncn4C)c3)nc2c1] |
| 304 | ADK-1 | 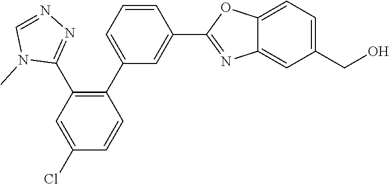 [SMILES: Cn1cnnc1-c1cc(Cl)ccc1-c1cccc(-c2nc3cc(CO)ccc3o2)c1] |
| 305 | ADL-2 | 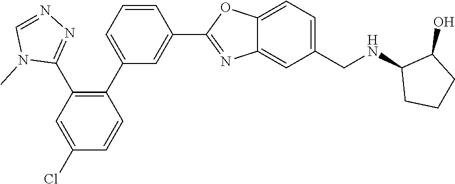 [SMILES: Cn1cnnc1-c1cc(Cl)ccc1-c1cccc(-c2nc3cc(CN[C@@H]4CCC[C@@H]4O)ccc3o2)c1] |
| 306 | ADM-14 | 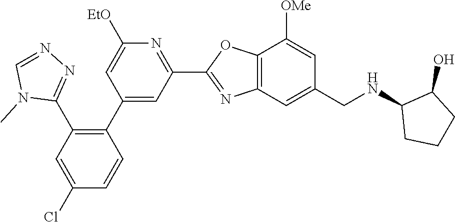 [SMILES: CCOc1cc(-c2ccc(Cl)cc2-c2nncn2C)cc(-c2nc3cc(CN[C@@H]4CCC[C@@H]4O)cc(OC)c3o2)n1] |
| 307 | ADN-1 | 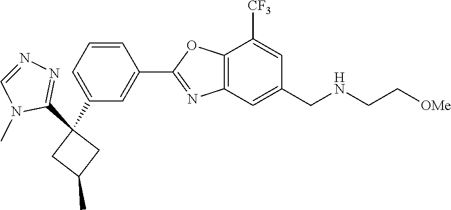 [SMILES: COCCNCc1cc(C(F)(F)F)c2oc(-c3cccc([C@]4(c5nncn5C)C[C@@H](C)C4)c3)nc2c1] |
| 308 | ADO-1 | 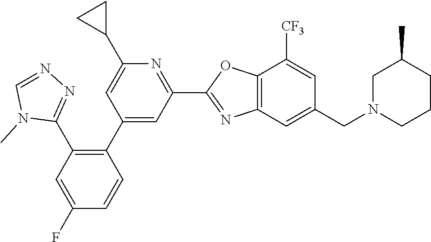 [SMILES: C[C@H]1CCCN(Cc2cc(C(F)(F)F)c3oc(-c4cc(-c5ccc(F)cc5-c5nncn5C)cc(C5CC5)n4)nc3c2)C1] |
| 309 | ADP-1 | 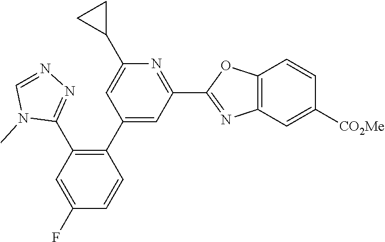 [SMILES: COC(=O)c1ccc2oc(-c3cc(-c4ccc(F)cc4-c4nncn4C)cc(C4CC4)n3)nc2c1] |
| 310 | ADQ-1 | 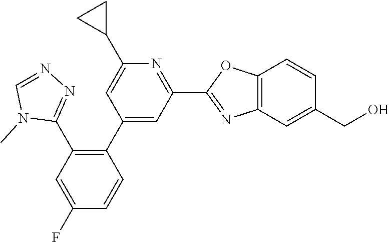 [SMILES: Cn1cnnc1-c1cc(F)ccc1-c1cc(-c2nc3cc(CO)ccc3o2)nc(C2CC2)c1] |
| 311 | ADR-1 | 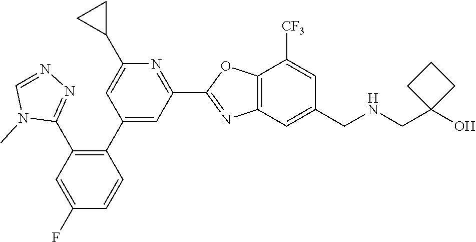 [SMILES: Cn1cnnc1-c1cc(F)ccc1-c1cc(-c2nc3cc(CNCC4(O)CCC4)cc(C(F)(F)F)c3o2)nc(C2CC2)c1] |
| 312 | ADS-1 | 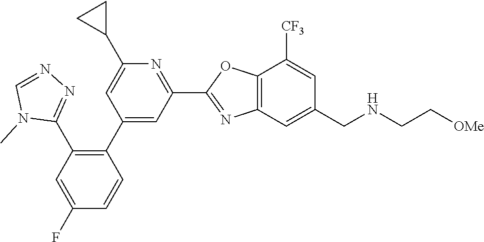 [SMILES: COCCNCc1cc(C(F)(F)F)c2oc(-c3cc(-c4ccc(F)cc4-c4nncn4C)cc(C4CC4)n3)nc2c1] |
| 313 | ADT-12 | 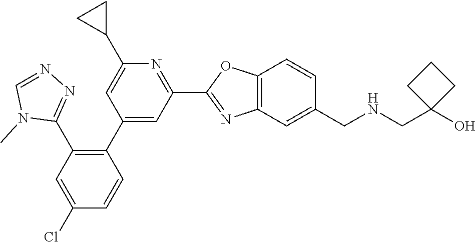 [SMILES: Cn1cnnc1-c1cc(Cl)ccc1-c1cc(-c2nc3cc(CNCC4(O)CCC4)ccc3o2)nc(C2CC2)c1] |
| 314 | ADU-2 | 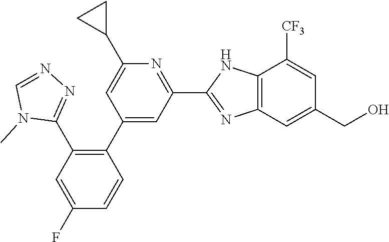 [SMILES: Cn1cnnc1-c1cc(F)ccc1-c1cc(-c2nc3cc(CO)cc(C(F)(F)F)c3[nH]2)nc(C2CC2)c1] |
| 315 | ADV-2 | 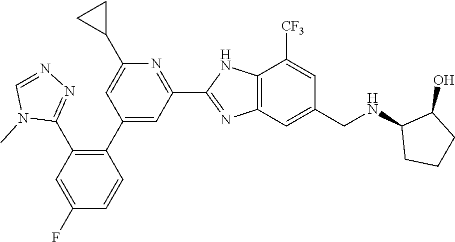 [SMILES: Cn1cnnc1-c1cc(F)ccc1-c1cc(-c2nc3cc(CN[C@@H]4CCC[C@@H]4O)cc(C(F)(F)F)c3[nH]2)nc(C2CC2)c1] |
| 316 | ADW-1 | 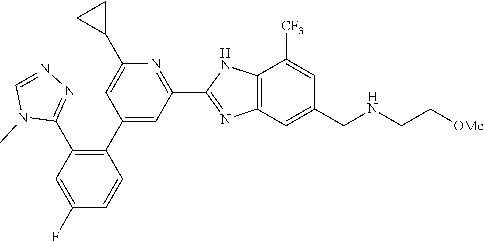 [SMILES: COCCNCc1cc(C(F)(F)F)c2[nH]c(-c3cc(-c4ccc(F)cc4-c4nncn4C)cc(C4CC4)n3)nc2c1] |
| 317 | ADX-3 | 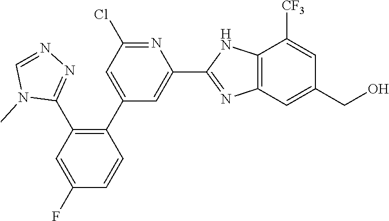 [SMILES: Cn1cnnc1-c1cc(F)ccc1-c1cc(Cl)nc(-c2nc3cc(CO)cc(C(F)(F)F)c3[nH]2)c1] |
| 318 | ADY-2 | 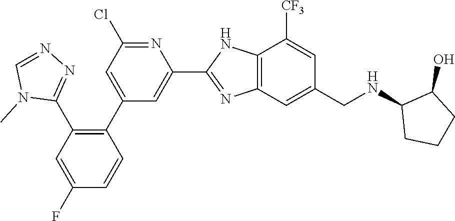 [SMILES: Cn1cnnc1-c1cc(F)ccc1-c1cc(Cl)nc(-c2nc3cc(CN[C@@H]4CCC[C@@H]4O)cc(C(F)(F)F)c3[nH]2)c1] |
| 319 | ADZ-1 | 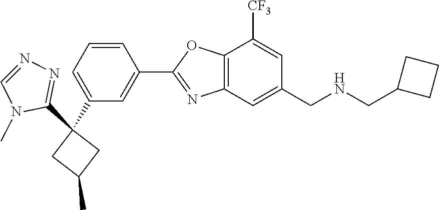 [SMILES: Cn1cnnc1[C@]1(c2cccc(-c3nc4cc(CNCC5CCC5)cc(C(F)(F)F)c4o3)c2)C[C@H](C)C1] |
| 320 | AEA-1 | 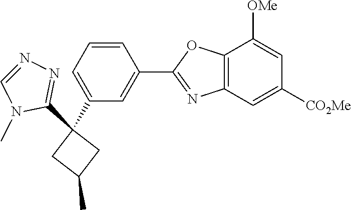 [SMILES: COC(=O)c1cc(OC)c2oc(-c3cccc([C@]4(c5nncn5C)C[C@@H](C)C4)c3)nc2c1] |
| 321 | AEB-1 | 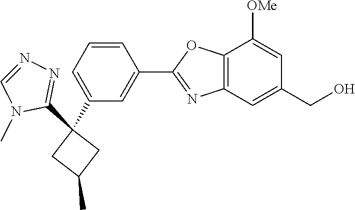 [SMILES: COc1cc(CO)cc2nc(-c3cccc([C@]4(c5nncn5C)C[C@@H](C)C4)c3)oc12] |
| 322 | AEC-2 | 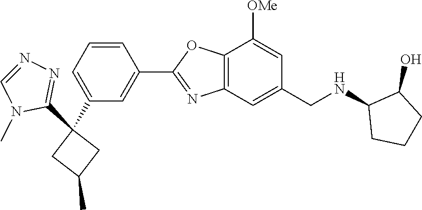 [SMILES: COc1cc(CN[C@@H]2CCC[C@@H]2O)cc2nc(-c3cccc([C@]4(c5nncn5C)C[C@@H](C)C4)c3)oc12] |
| 323 | AED-3 | 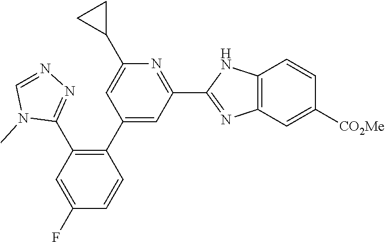 [SMILES: COC(=O)c1ccc2[nH]c(-c3cc(-c4ccc(F)cc4-c4nncn4C)cc(C4CC4)n3)nc2c1] |
| 324 | AEE-1 | 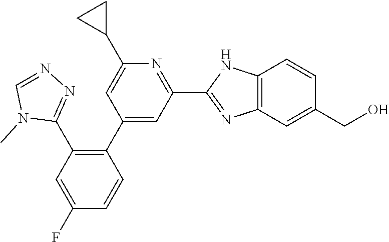 [SMILES: Cn1cnnc1-c1cc(F)ccc1-c1cc(-c2nc3cc(CO)ccc3[nH]2)nc(C2CC2)c1] |
| 325 | AEF-2 | 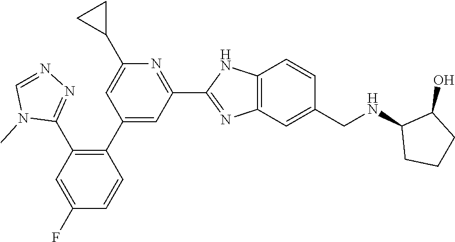 [SMILES: Cn1cnnc1-c1cc(F)ccc1-c1cc(-c2nc3cc(CN[C@@H]4CCC[C@@H]4O)ccc3[nH]2)nc(C2CC2)c1] |
| 326 | AEG-8 | 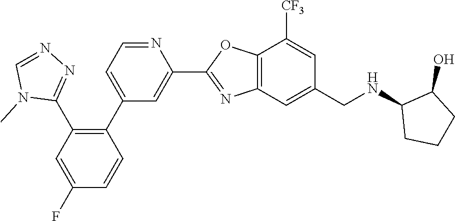 [SMILES: Cn1cnnc1-c1cc(F)ccc1-c1ccnc(-c2nc3cc(CN[C@@H]4CCC[C@@H]4O)cc(C(F)(F)F)c3o2)c1] |
| 327 | AEH-2 | 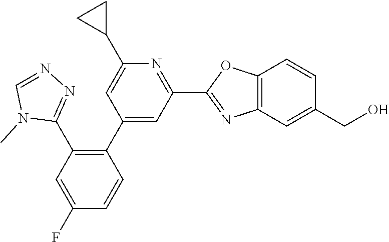 [SMILES: Cn1cnnc1-c1cc(F)ccc1-c1cc(-c2nc3cc(CO)ccc3o2)nc(C2CC2)c1] |
| 328 | AEI-1 | 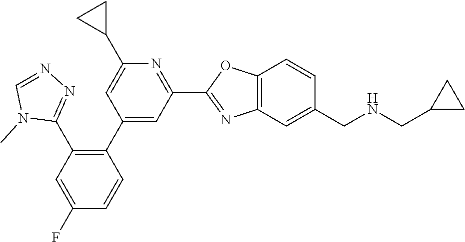 [SMILES: Cn1cnnc1-c1cc(F)ccc1-c1cc(-c2nc3cc(CNCC4CC4)ccc3o2)nc(C2CC2)c1] |
| 329 | AEJ-4 | 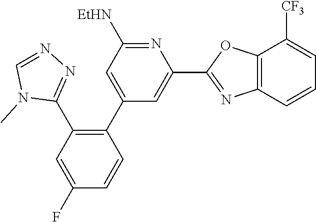 |
| 330 | AEK-2 | 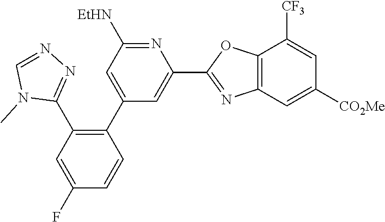 |
| 331 | AEL-1 | 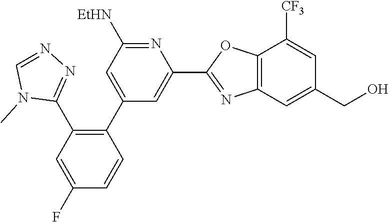 |
| 332 | AHN-2 | 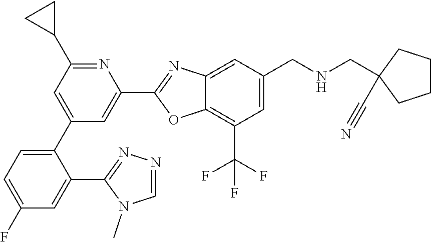 [SMILES: Cn1cnnc1-c1cc(F)ccc1-c1cc(-c2nc3cc(CNCC4(C#N)CCCC4)cc(C(F)(F)F)c3o2)nc(C2CC2)c1] |
| 333 | AFY-2 | 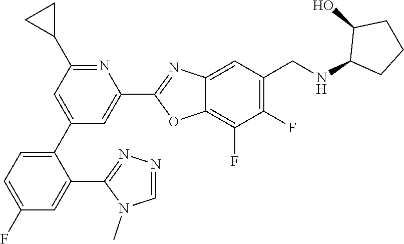 [SMILES: Cn1cnnc1-c1cc(F)ccc1-c1cc(-c2nc3cc(CN[C@@H]4CCC[C@@H]4O)c(F)c(F)c3o2)nc(C2CC2)c1] |
| 334 | AGB-1 | 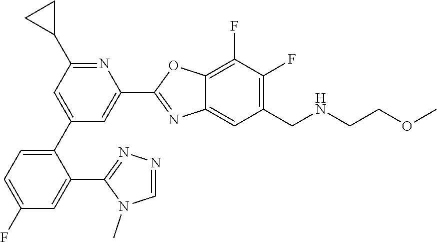 [SMILES: COCCNCc1cc2nc(-c3cc(-c4ccc(F)cc4-c4nncn4C)cc(C4CC4)n3)oc2c(F)c1F] |
| 335 | AHL-7 | 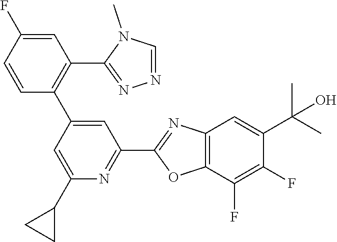 [SMILES: Cn1cnnc1-c1cc(F)ccc1-c1cc(-c2nc3cc(C(C)(C)O)c(F)c(F)c3o2)nc(C2CC2)c1] |
| 336 | AHM-3 | 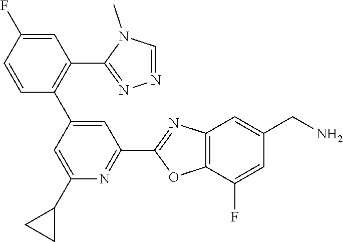 [SMILES: Cn1cnnc1-c1cc(F)ccc1-c1cc(-c2nc3cc(CN)cc(F)c3o2)nc(C2CC2)c1] |
| 337 | AHH-3 | 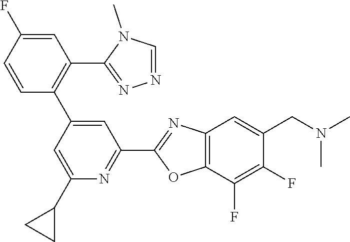 [SMILES: CN(C)Cc1cc2nc(-c3cc(-c4ccc(F)cc4-c4nncn4C)cc(C4CC4)n3)oc2c(F)c1F] |
| 338 | AHI-5 | 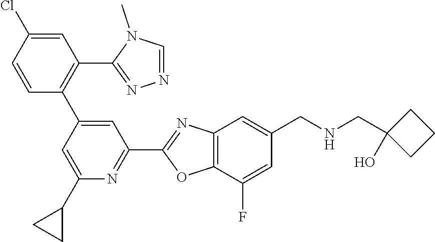 [SMILES: Cn1cnnc1-c1cc(Cl)ccc1-c1cc(-c2nc3cc(CNCC4(O)CCC4)cc(F)c3o2)nc(C2CC2)c1] |
| 339 | AHJ-1 | 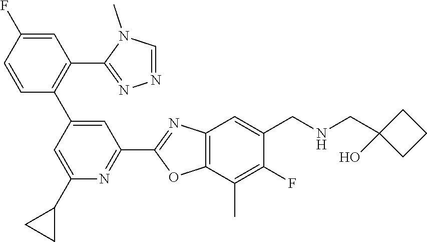 [SMILES: Cc1c(F)c(CNCC2(O)CCC2)cc2nc(-c3cc(-c4ccc(F)cc4-c4nncn4C)cc(C4CC4)n3)oc12] |
| 340 | AHK-4 | 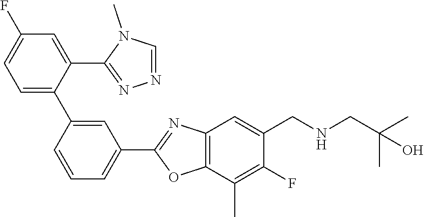 [SMILES: Cc1c(F)c(CNCC(C)(C)O)cc2nc(-c3cccc(-c4ccc(F)cc4-c4nncn4C)c3)oc12] |
| 341 | JO-1 | 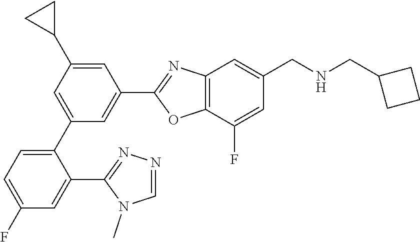 [SMILES: Cn1cnnc1-c1cc(F)ccc1-c1cc(-c2nc3cc(CNCC4CCC4)cc(F)c3o2)cc(C2CC2)c1] |
| 342 | AHF-1 | 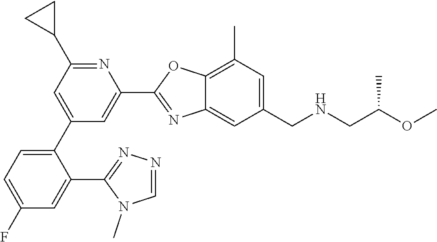 [SMILES: CO[C@@H](C)CNCc1cc(C)c2oc(-c3cc(-c4ccc(F)cc4-c4nncn4C)cc(C4CC4)n3)nc2c1] |
| 343 | AHG-1 | 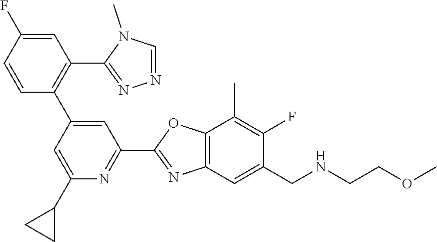 [SMILES: COCCNCc1cc2nc(-c3cc(-c4ccc(F)cc4-c4nncn4C)cc(C4CC4)n3)oc2c(C)c1F] |
| 344 | AGF-1 | 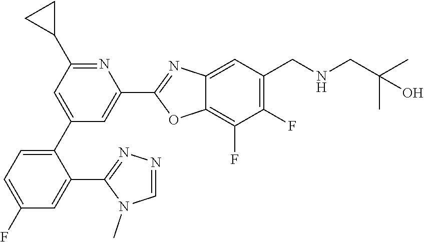 [SMILES: Cn1cnnc1-c1cc(F)ccc1-c1cc(-c2nc3cc(CNCC(C)(C)O)c(F)c(F)c3o2)nc(C2CC2)c1] |
| 345 | AHC-1 | 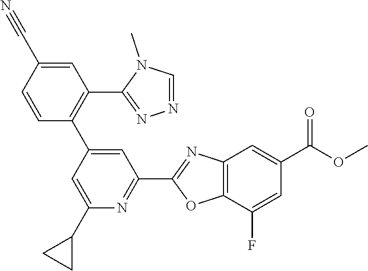 [SMILES: COC(=O)c1cc(F)c2oc(-c3cc(-c4ccc(C#N)cc4-c4nncn4C)cc(C4CC4)n3)nc2c1] |
| 346 | AHD-1 | 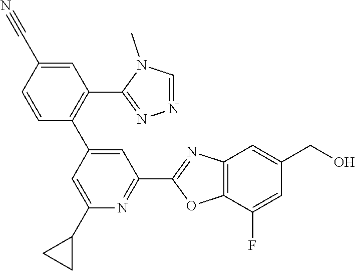 [SMILES: Cn1cnnc1-c1cc(C#N)ccc1-c1cc(-c2nc3cc(CO)cc(F)c3o2)nc(C2CC2)c1] |
| 347 | AHA-2 | 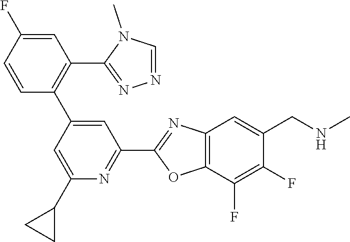 [SMILES: CNCc1cc2nc(-c3cc(-c4ccc(F)cc4-c4nncn4C)cc(C4CC4)n3)oc2c(F)c1F] |
| 348 | AGZ-2 | 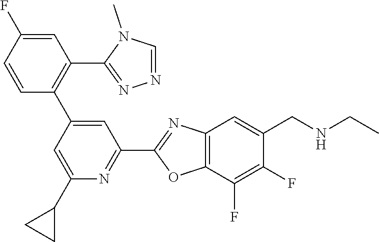 [SMILES: CCNCc1cc2nc(-c3cc(-c4ccc(F)cc4-c4nncn4C)cc(C4CC4)n3)oc2c(F)c1F] |
| 349 | AHB-1 | 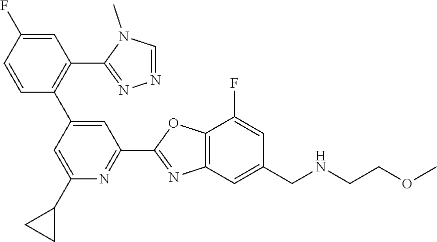 [SMILES: COCCNCc1cc(F)c2oc(-c3cc(-c4ccc(F)cc4-c4nncn4C)cc(C4CC4)n3)nc2c1] |
| 350 | AHE-2 | 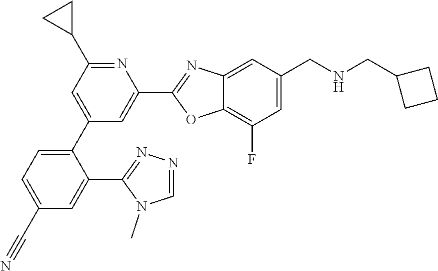 [SMILES: Cn1cnnc1-c1cc(C#N)ccc1-c1cc(-c2nc3cc(CNCC4CCC4)cc(F)c3o2)nc(C2CC2)c1] |
| 351 | AGX-1 | 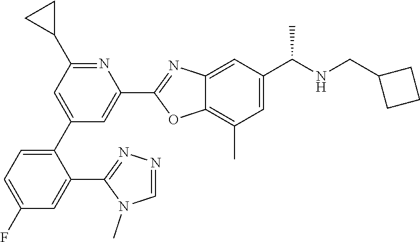 [SMILES: Cc1cc([C@H](C)NCC2CCC2)cc2nc(-c3cc(-c4ccc(F)cc4-c4nncn4C)cc(C4CC4)n3)oc12] |
| 352 | AGY-1 | 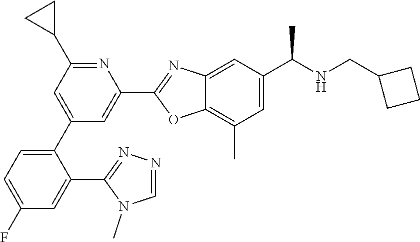 [SMILES: Cc1cc([C@@H](C)NCC2CCC2)cc2nc(-c3cc(-c4ccc(F)cc4-c4nncn4C)cc(C4CC4)n3)oc12] |
| 353 | IT-1 | 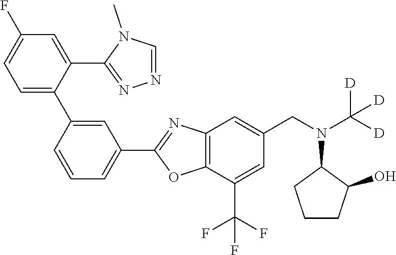 [SMILES: [2H]C([2H])([2H])N(Cc1cc(C(F)(F)F)c2oc(-c3cccc(-c4ccc(F)cc4-c4nncn4C)c3)nc2c1)[C@@H]1CCC[C@@H]1O] |
| 354 | JR-2 | 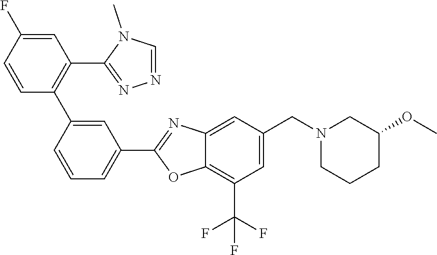 [SMILES: CO[C@@H]1CCCN(Cc2cc(C(F)(F)F)c3oc(-c4cccc(-c5ccc(F)cc5-c5nncn5C)c4)nc3c2)C1] |
| 355 | JS-1 | 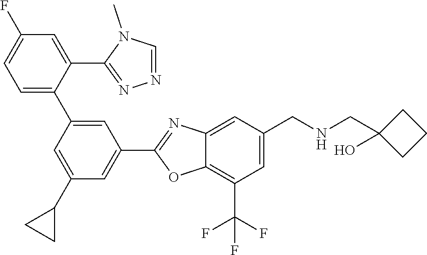 [SMILES: Cn1cnnc1-c1cc(F)ccc1-c1cc(-c2nc3cc(CNCC4(O)CCC4)cc(C(F)(F)F)c3o2)cc(C2CC2)c1] |
| 356 | JT-1 | 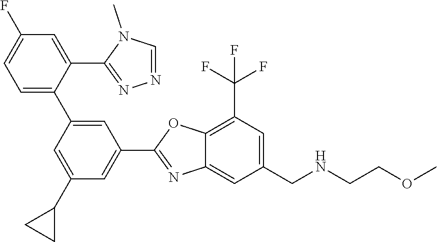 [SMILES: COCCNCc1cc(C(F)(F)F)c2oc(-c3cc(-c4ccc(F)cc4-c4nncn4C)cc(C4CC4)c3)nc2c1] |
| 357 | JU-1 | 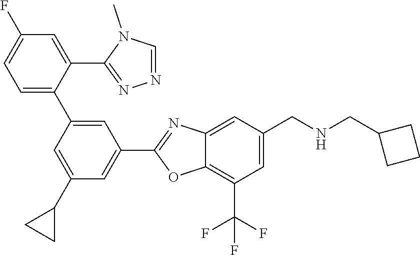 [SMILES: Cn1cnnc1-c1cc(F)ccc1-c1cc(-c2nc3cc(CNCC4CCC4)cc(C(F)(F)F)c3o2)cc(C2CC2)c1] |
| 358 | AGU-1 | 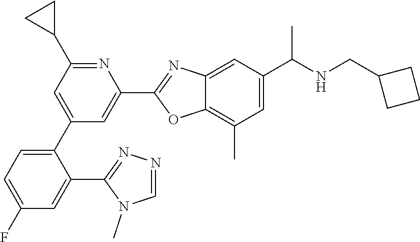 [SMILES: Cc1cc(C(C)NCC2CCC2)cc2nc(-c3cc(-c4ccc(F)cc4-c4nncn4C)cc(C4CC4)n3)oc12] |
| 359 | JL-1 | 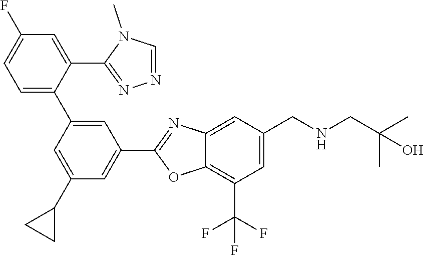 [SMILES: Cn1cnnc1-c1cc(F)ccc1-c1cc(-c2nc3cc(CNCC(C)(C)O)cc(C(F)(F)F)c3o2)cc(C2CC2)c1] |
| 360 | JM-4 | 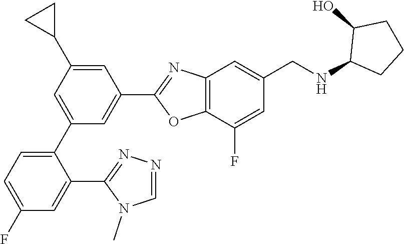 [SMILES: Cn1cnnc1-c1cc(F)ccc1-c1cc(-c2nc3cc(CN[C@@H]4CCC[C@@H]4O)cc(F)c3o2)cc(C2CC2)c1] |
| 361 | JN-1 | 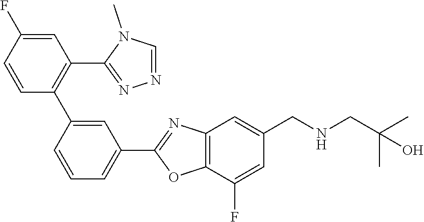 [SMILES: Cn1cnnc1-c1cc(F)ccc1-c1cccc(-c2nc3cc(CNCC(C)(C)O)cc(F)c3o2)c1] |
| 362 | JP-1 | 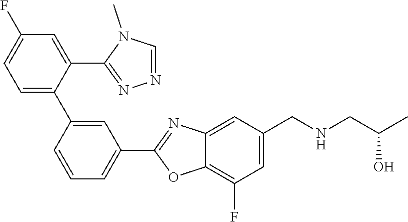 [SMILES: C[C@H](O)CNCc1cc(F)c2oc(-c3cccc(-c4ccc(F)cc4-c4nncn4C)c3)nc2c1] |
| 363 | JQ-1 | 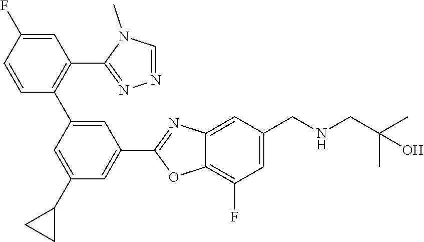 [SMILES: Cn1cnnc1-c1cc(F)ccc1-c1cc(-c2nc3cc(CNCC(C)(C)O)cc(F)c3o2)cc(C2CC2)c1] |
| 364 | AGS-4 | 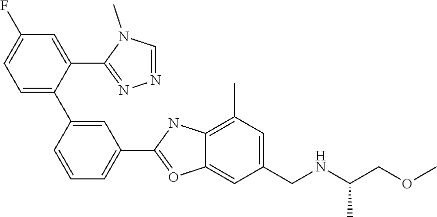 |
| 365 | AGT-1 | 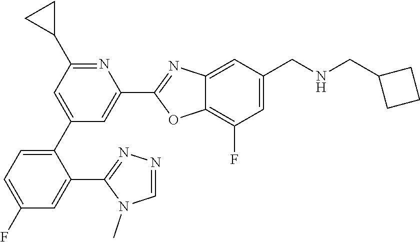 [SMILES: Cn1cnnc1-c1cc(F)ccc1-c1cc(-c2nc3cc(CNCC4CCC4)cc(F)c3o2)nc(C2CC2)c1] |
| 366 | AFI-1 | 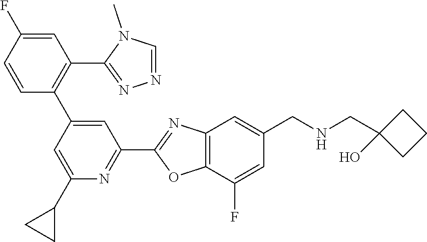 [SMILES: Cn1cnnc1-c1cc(F)ccc1-c1cc(-c2nc3cc(CNCC4(O)CCC4)cc(F)c3o2)nc(C2CC2)c1] |
| 367 | AFQ-6 | 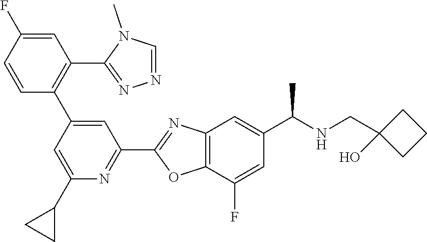 [SMILES: C[C@@H](NCC1(O)CCC1)c1cc(F)c2oc(-c3cc(-c4ccc(F)cc4-c4nncn4C)cc(C4CC4)n3)nc2c1] |
| 368 | AFR-1 | 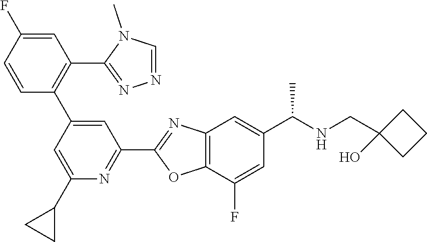 [SMILES: C[C@H](NCC1(O)CCC1)c1cc(F)c2oc(-c3cc(-c4ccc(F)cc4-c4nncn4C)cc(C4CC4)n3)nc2c1] |
| 369 | AGP-7 | 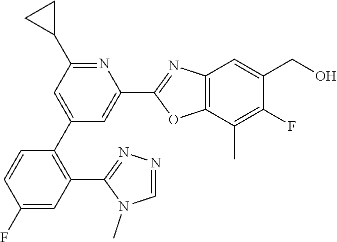 [SMILES: Cc1c(F)c(CO)cc2nc(-c3cc(-c4ccc(F)cc4-c4nncn4C)cc(C4CC4)n3)oc12] |
| 370 | AGR-9 | 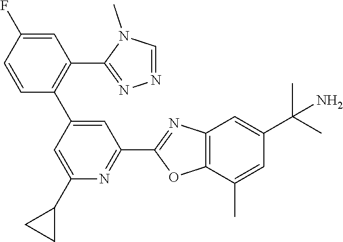 [SMILES: Cc1cc(C(C)(C)N)cc2nc(-c3cc(-c4ccc(F)cc4-c4nncn4C)cc(C4CC4)n3)oc12] |
| 371 | AEO-1 | 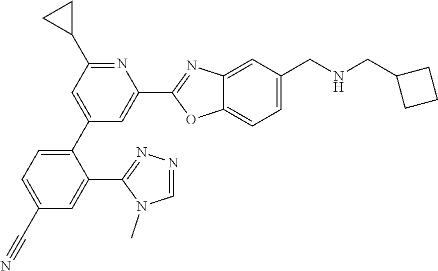 [SMILES: Cn1cnnc1-c1cc(C#N)ccc1-c1cc(-c2nc3cc(CNCC4CCC4)ccc3o2)nc(C2CC2)c1] |
| 372 | AGQ-2 | 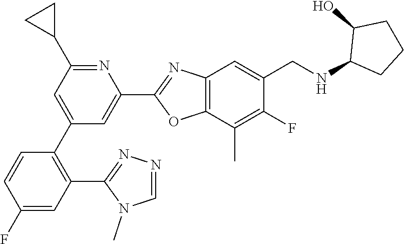 [SMILES: Cc1c(F)c(CN[C@@H]2CCC[C@@H]2O)cc2nc(-c3cc(-c4ccc(F)cc4-c4nncn4C)cc(C4CC4)n3)oc12] |
| 373 | JI-3 | 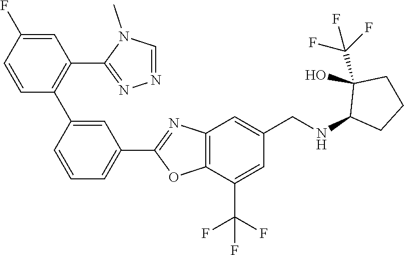 [SMILES: Cn1cnnc1-c1cc(F)ccc1-c1cccc(-c2nc3cc(CN[C@@H]4CCC[C@@]4(O)C(F)(F)F)cc(C(F)(F)F)c3o2)c1] |
| 374 | JJ-3 | 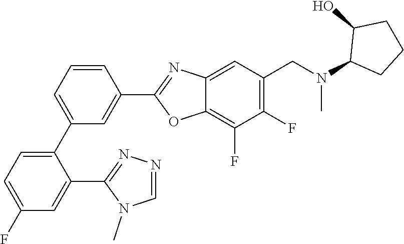 [SMILES: CN(Cc1cc2nc(-c3cccc(-c4ccc(F)cc4-c4nncn4C)c3)oc2c(F)c1F)[C@@H]1CCC[C@@H]1O] |
| 375 | JK-7 | 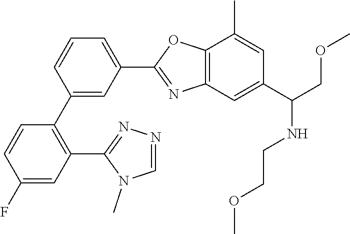 [SMILES: COCCNC(COC)c1cc(C)c2oc(-c3cccc(-c4ccc(F)cc4-c4nncn4C)c3)nc2c1] |
| 376 | AEZ-1 | 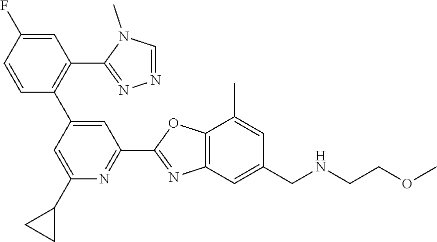 [SMILES: COCCNCc1cc(C)c2oc(-c3cc(-c4ccc(F)cc4-c4nncn4C)cc(C4CC4)n3)nc2c1] |
| 377 | AGO-2 | 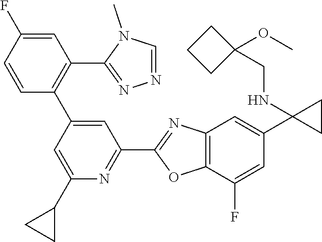 [SMILES: COC1(CNC2(c3cc(F)c4oc(-c5cc(-c6ccc(F)cc6-c6nncn6C)cc(C6CC6)n5)nc4c3)CC2)CCC1] |
| 378 | JG-1 | 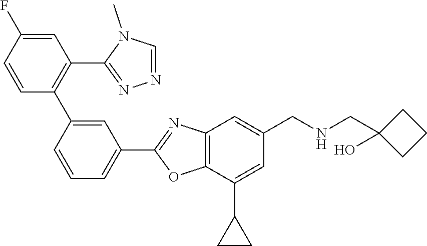 [SMILES: Cn1cnnc1-c1cc(F)ccc1-c1cccc(-c2nc3cc(CNCC4(O)CCC4)cc(C4CC4)c3o2)c1] |
| 379 | JH-1 | 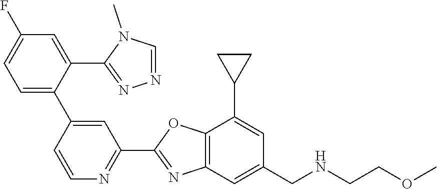 [SMILES: COCCNCc1cc(C2CC2)c2oc(-c3cc(-c4ccc(F)cc4-c4nncn4C)ccn3)nc2c1] |
| 380 | AGM-8 | 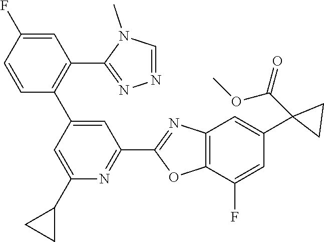 [SMILES: COC(=O)C1(c2cc(F)c3oc(-c4cc(-c5ccc(F)cc5-c5nncn5C)cc(C5CC5)n4)nc3c2)CC1] |
| 381 | AGN-3 | 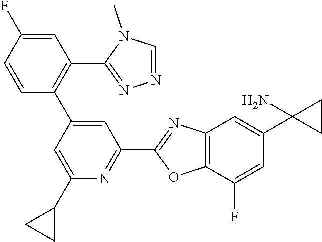 [SMILES: Cn1cnnc1-c1cc(F)ccc1-c1cc(-c2nc3cc(C4(N)CC4)cc(F)c3o2)nc(C2CC2)c1] |
| 382 | JB-2 | 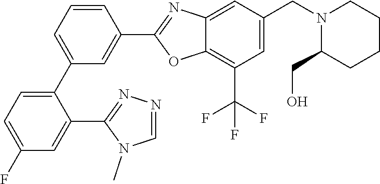 [SMILES: Cn1cnnc1-c1cc(F)ccc1-c1cccc(-c2nc3cc(CN4CCCC[C@H]4CO)cc(C(F)(F)F)c3o2)c1] |
| 383 | JC-1 | 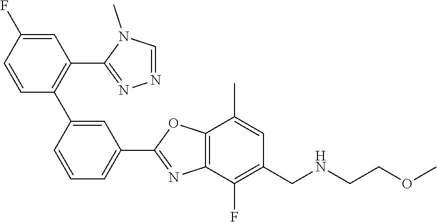 [SMILES: COCCNCc1cc(C)c2oc(-c3cccc(-c4ccc(F)cc4-c4nncn4C)c3)nc2c1F] |
| 384 | JD-1 | 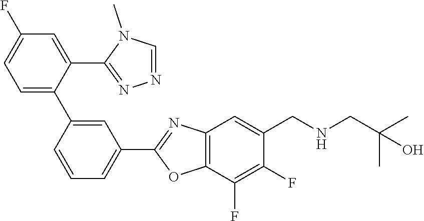 [SMILES: Cn1cnnc1-c1cc(F)ccc1-c1cccc(-c2nc3cc(CNCC(C)(C)O)c(F)c(F)c3o2)c1] |
| 385 | JE-1 | 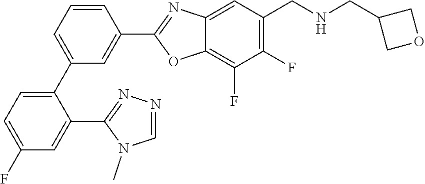 [SMILES: Cn1cnnc1-c1cc(F)ccc1-c1cccc(-c2nc3cc(CNCC4COC4)c(F)c(F)c3o2)c1] |
| 386 | JF-1 | 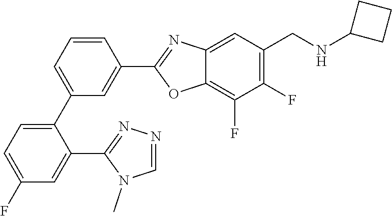 [SMILES: Cn1cnnc1-c1cc(F)ccc1-c1cccc(-c2nc3cc(CNC4CCC4)c(F)c(F)c3o2)c1] |
| 387 | AGG-2 | 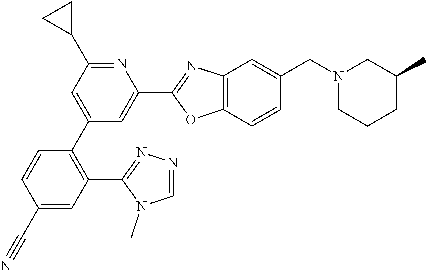 [SMILES: C[C@H]1CCCN(Cc2ccc3oc(-c4cc(-c5ccc(C#N)cc5-c5nncn5C)cc(C5CC5)n4)nc3c2)C1] |
| 388 | AGI-1 | 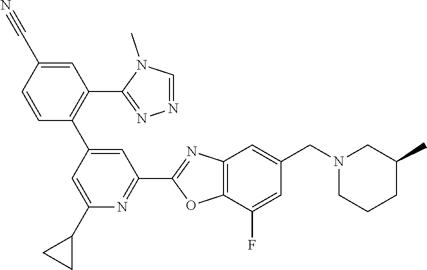 [SMILES: C[C@H]1CCCN(Cc2cc(F)c3oc(-c4cc(-c5ccc(C#N)cc5-c5nncn5C)cc(C5CC5)n4)nc3c2)C1] |
| 389 | AGH-4 | 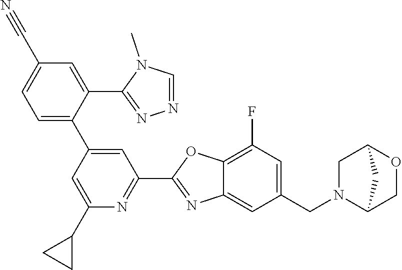 [SMILES: Cn1cnnc1-c1cc(C#N)ccc1-c1cc(-c2nc3cc(CN4C[C@@H]5C[C@H]4CO5)cc(F)c3o2)nc(C2CC2)c1] |
| 390 | AGJ-1 | 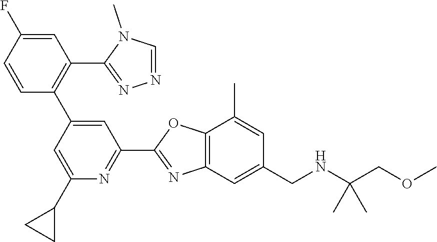 [SMILES: COCC(C)(C)NCc1cc(C)c2oc(-c3cc(-c4ccc(F)cc4-c4nncn4C)cc(C4CC4)n3)nc2c1] |
| 391 | AGK-1 | 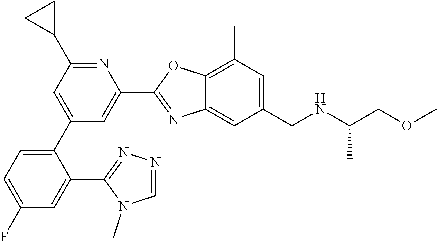 [SMILES: COC[C@H](C)NCc1cc(C)c2oc(-c3cc(-c4ccc(F)cc4-c4nncn4C)cc(C4CC4)n3)nc2c1] |
| 392 | AGL-1 | 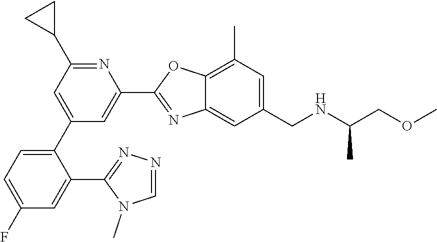 [SMILES: COC[C@@H](C)NCc1cc(C)c2oc(-c3cc(-c4ccc(F)cc4-c4nncn4C)cc(C4CC4)n3)nc2c1] |
| 393 | IZ-1 | 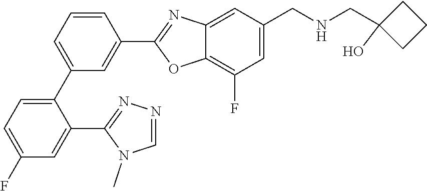 [SMILES: Cn1cnnc1-c1cc(F)ccc1-c1cccc(-c2nc3cc(CNCC4(O)CCC4)cc(F)c3o2)c1] |
| 394 | IY-1 | 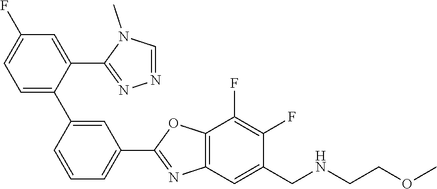 [SMILES: COCCNCc1cc2nc(-c3cccc(-c4ccc(F)cc4-c4nncn4C)c3)oc2c(F)c1F] |
| 395 | IU-2 | 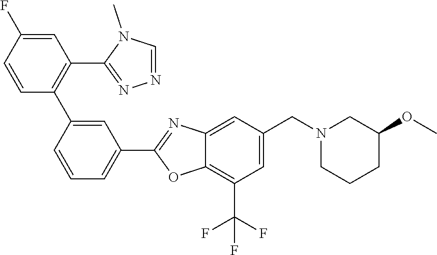 [SMILES: CO[C@H]1CCCN(Cc2cc(C(F)(F)F)c3oc(-c4cccc(-c5ccc(F)cc5-c5nncn5C)c4)nc3c2)C1] |
| 396 | IV-2 | 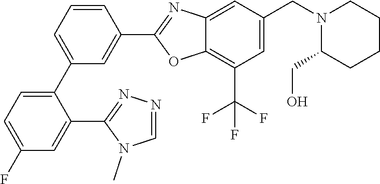 [SMILES: Cn1cnnc1-c1cc(F)ccc1-c1cccc(-c2nc3cc(CN4CCCC[C@@H]4CO)cc(C(F)(F)F)c3o2)c1] |
| 397 | IW-1 | 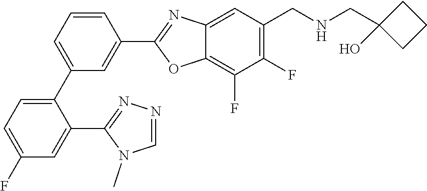 [SMILES: Cn1cnnc1-c1cc(F)ccc1-c1cccc(-c2nc3cc(CNCC4(O)CCC4)c(F)c(F)c3o2)c1] |
| 398 | IX-1 | 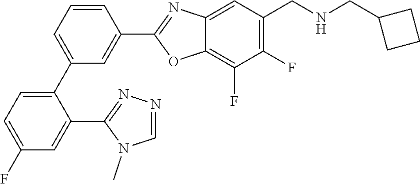 [SMILES: Cn1cnnc1-c1cc(F)ccc1-c1cccc(-c2nc3cc(CNCC4CCC4)c(F)c(F)c3o2)c1] |
| 399 | AGD-1 | 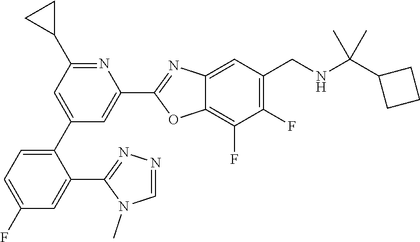 [SMILES: Cn1cnnc1-c1cc(F)ccc1-c1cc(-c2nc3cc(CNC(C)(C)C4CCC4)c(F)c(F)c3o2)nc(C2CC2)c1] |
| 400 | AGE-1 | 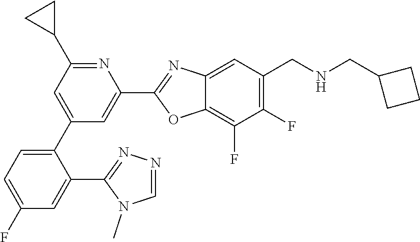 [SMILES: Cn1cnnc1-c1cc(F)ccc1-c1cc(-c2nc3cc(CNCC4CCC4)c(F)c(F)c3o2)nc(C2CC2)c1] |
| 401 | IS-1 | 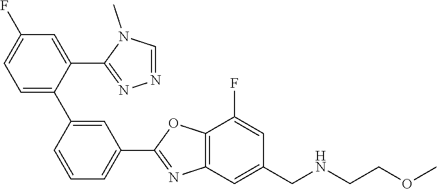 [SMILES: COCCNCc1cc(F)c2oc(-c3cccc(-c4ccc(F)cc4-c4nncn4C)c3)nc2c1] |
| 402 | AGA-1 | 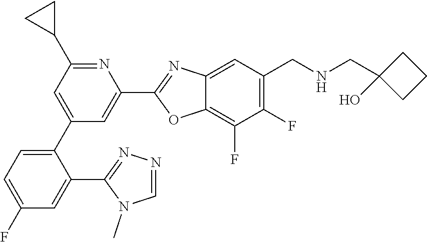 [SMILES: Cn1cnnc1-c1cc(F)ccc1-c1cc(-c2nc3cc(CNCC4(O)CCC4)c(F)c(F)c3o2)nc(C2CC2)c1] |
| 403 | AGC-2 | 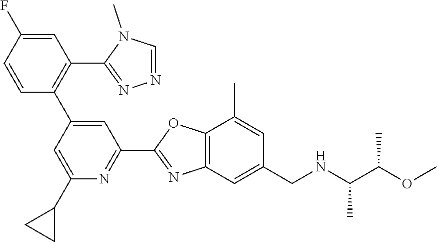 [SMILES: CO[C@@H](C)[C@H](C)NCc1cc(C)c2oc(-c3cc(-c4ccc(F)cc4-c4nncn4C)cc(C4CC4)n3)nc2c1] |
| 404 | IR-1 | 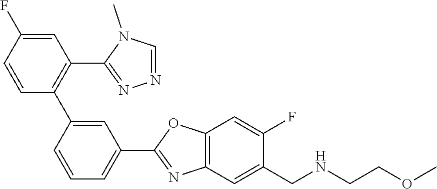 [SMILES: COCCNCc1cc2nc(-c3cccc(-c4ccc(F)cc4-c4nncn4C)c3)oc2cc1F] |
| 405 | AFX-1 | 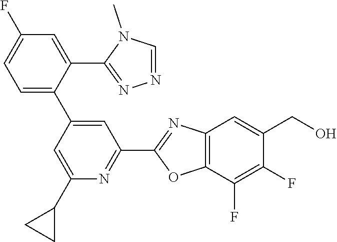 [SMILES: Cn1cnnc1-c1cc(F)ccc1-c1cc(-c2nc3cc(CO)c(F)c(F)c3o2)nc(C2CC2)c1] |
| 406 | AFZ-2 | 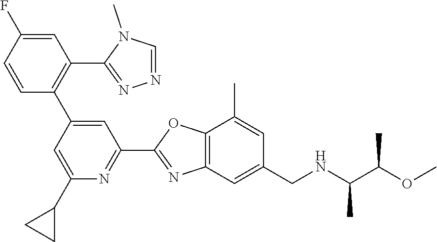 [SMILES: CO[C@H](C)[C@@H](C)NCc1cc(C)c2oc(-c3cc(-c4ccc(F)cc4-c4nncn4C)cc(C4CC4)n3)nc2c1] |
| 407 | IP-7 | 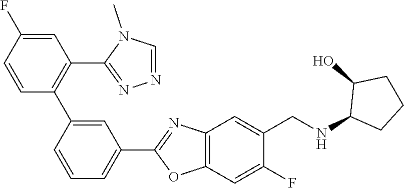 [SMILES: Cn1cnnc1-c1cc(F)ccc1-c1cccc(-c2nc3cc(CN[C@@H]4CCC[C@@H]4O)c(F)cc3o2)c1] |
| 408 | IQ-8 | 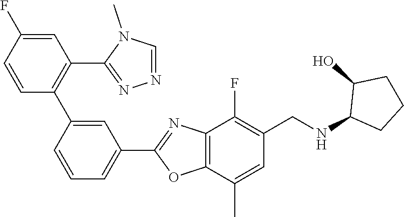 [SMILES: Cc1cc(CN[C@@H]2CCC[C@@H]2O)c(F)c2nc(-c3cccc(-c4ccc(F)cc4-c4nncn4C)c3)oc12] |
| 409 | AEU-2 | 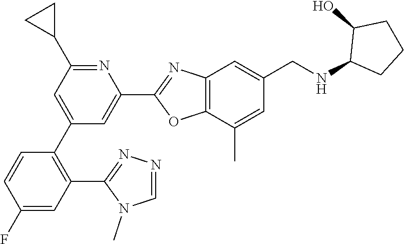 [SMILES: Cc1cc(CN[C@@H]2CCC[C@@H]2O)cc2nc(-c3cc(-c4ccc(F)cc4-c4nncn4C)cc(C4CC4)n3)oc12] |
| 410 | AFF-1 | 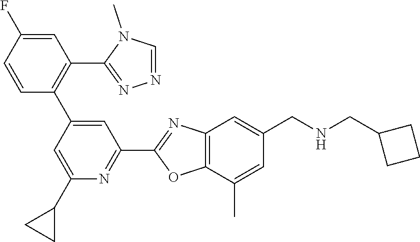 [SMILES: Cc1cc(CNCC2CCC2)cc2nc(-c3cc(-c4ccc(F)cc4-c4nncn4C)cc(C4CC4)n3)oc12] |
| 411 | AFU-1 | 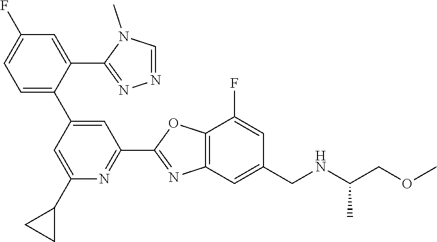 [SMILES: COC[C@H](C)NCc1cc(F)c2oc(-c3cc(-c4ccc(F)cc4-c4nncn4C)cc(C4CC4)n3)nc2c1] |
| 412 | AFV-1 | 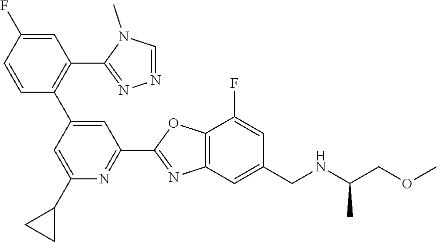 [SMILES: COC[C@@H](C)NCc1cc(F)c2oc(-c3cc(-c4ccc(F)cc4-c4nncn4C)cc(C4CC4)n3)nc2c1] |
| 413 | AFW-1 | 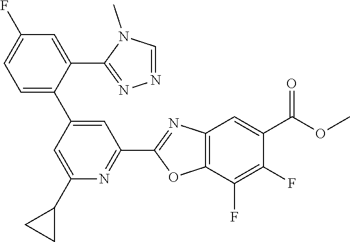 [SMILES: COC(=O)c1cc2nc(-c3cc(-c4ccc(F)cc4-c4nncn4C)cc(C4CC4)n3)oc2c(F)c1F] |
| 414 | DB-1 | 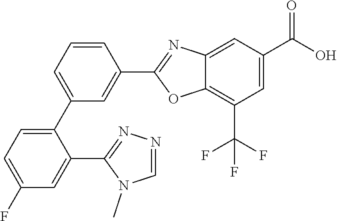 [SMILES: Cn1cnnc1-c1cc(F)ccc1-c1cccc(-c2nc3cc(C(=O)O)cc(C(F)(F)F)c3o2)c1] |
| 415 | IN-2 | 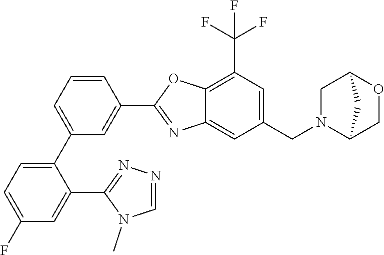 [SMILES: Cn1cnnc1-c1cc(F)ccc1-c1cccc(-c2nc3cc(CN4C[C@@H]5C[C@H]4CO5)cc(C(F)(F)F)c3o2)c1] |
| 416 | IO-4 | 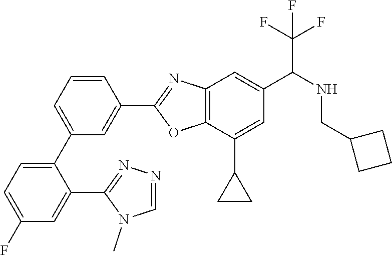 [SMILES: Cn1cnnc1-c1cc(F)ccc1-c1cccc(-c2nc3cc(C(NCC4CCC4)C(F)(F)F)cc(C4CC4)c3o2)c1] |
| 417 | AFS-2 | 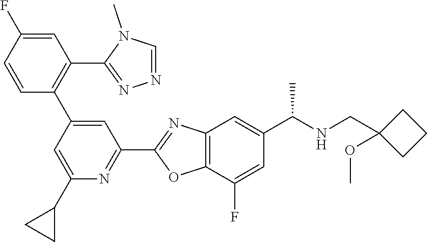 [SMILES: COC1(CN[C@@H](C)c2cc(F)c3oc(-c4cc(-c5ccc(F)cc5-c5nncn5C)cc(C5CC5)n4)nc3c2)CCC1] |
| 418 | AFT-1 | 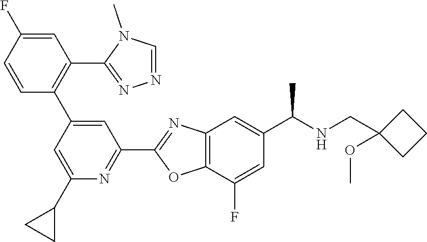 [SMILES: COC1(CN[C@H](C)c2cc(F)c3oc(-c4cc(-c5ccc(F)cc5-c5nncn5C)cc(C5CC5)n4)nc3c2)CCC1] |
| 419 | IL-6 | 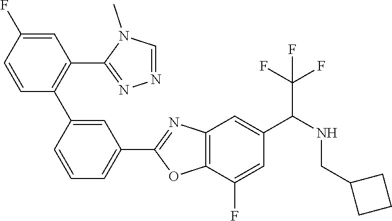 [SMILES: Cn1cnnc1-c1cc(F)ccc1-c1cccc(-c2nc3cc(C(NCC4CCC4)C(F)(F)F)cc(F)c3o2)c1] |
| 420 | IM-8 | 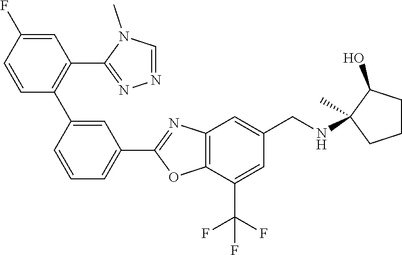 [SMILES: Cn1cnnc1-c1cc(F)ccc1-c1cccc(-c2nc3cc(CN[C@]4(C)CCC[C@@H]4O)cc(C(F)(F)F)c3o2)c1] |
| 421 | AFP-3 | 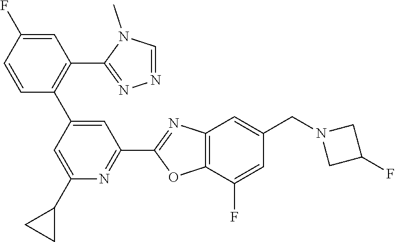 [SMILES: Cn1cnnc1-c1cc(F)ccc1-c1cc(-c2nc3cc(CN4CC(F)C4)cc(F)c3o2)nc(C2CC2)c1] |
| 422 | II-1 | 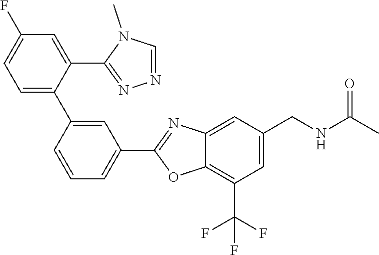 [SMILES: CC(=O)NCc1cc(C(F)(F)F)c2oc(-c3cccc(-c4ccc(F)cc4-c4nncn4C)c3)nc2c1] |
| 423 | IJ-1 | 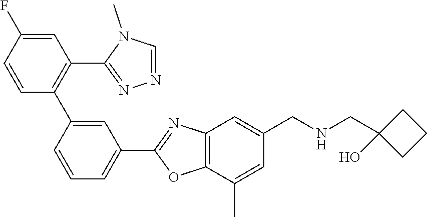 [SMILES: Cc1cc(CNCC2(O)CCC2)cc2nc(-c3cccc(-c4ccc(F)cc4-c4nncn4C)c3)oc12] |
| 424 | IK-1 | 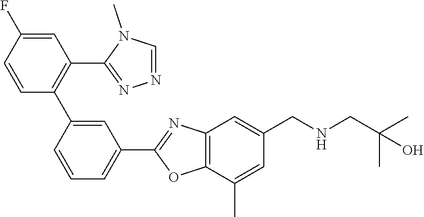 [SMILES: Cc1cc(CNCC(C)(C)O)cc2nc(-c3cccc(-c4ccc(F)cc4-c4nncn4C)c3)oc12] |
| 425 | AFK-1 | 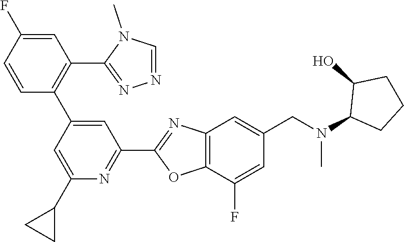 [SMILES: CN(Cc1cc(F)c2oc(-c3cc(-c4ccc(F)cc4-c4nncn4C)cc(C4CC4)n3)nc2c1)[C@@H]1CCC[C@@H]1O] |
| 426 | AFL-2 | 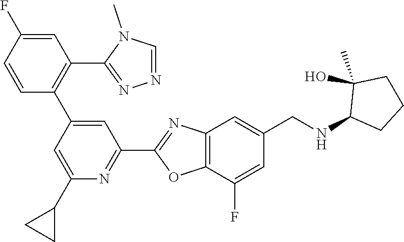 [SMILES: Cn1cnnc1-c1cc(F)ccc1-c1cc(-c2nc3cc(CN[C@@H]4CCC[C@]4(C)O)cc(F)c3o2)nc(C2CC2)c1] |
| 427 | AFN-2 | 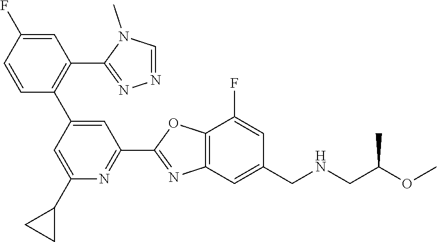 [SMILES: CO[C@H](C)CNCc1cc(F)c2oc(-c3cc(-c4ccc(F)cc4-c4nncn4C)cc(C4CC4)n3)nc2c1] |
| 428 | AFO-2 | 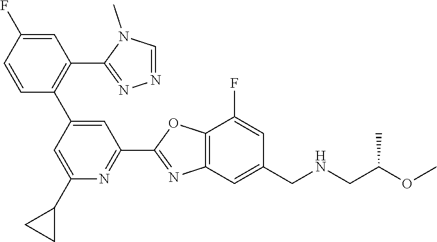 [SMILES: CO[C@@H](C)CNCc1cc(F)c2oc(-c3cc(-c4ccc(F)cc4-c4nncn4C)cc(C4CC4)n3)nc2c1] |
| 429 | AFM-1 | 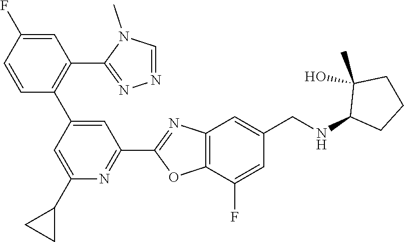 [SMILES: Cn1cnnc1-c1cc(F)ccc1-c1cc(-c2nc3cc(CN[C@@H]4CCC[C@@]4(C)O)cc(F)c3o2)nc(C2CC2)c1] |
| 430 | AFA-2 | 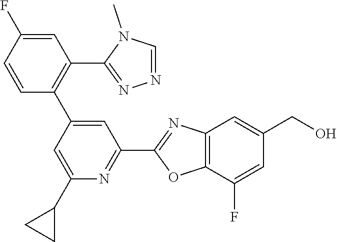 [SMILES: Cn1cnnc1-c1cc(F)ccc1-c1cc(-c2nc3cc(CO)cc(F)c3o2)nc(C2CC2)c1] |
| 431 | IH-9 | 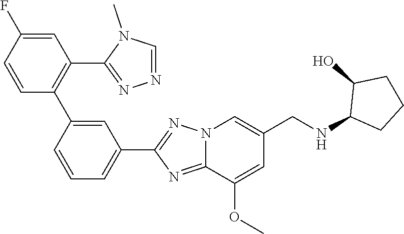 [SMILES: COc1cc(CN[C@@H]2CCC[C@@H]2O)cn2nc(-c3cccc(-c4ccc(F)cc4-c4nncn4C)c3)nc12] |
| 432 | AFB-2 | 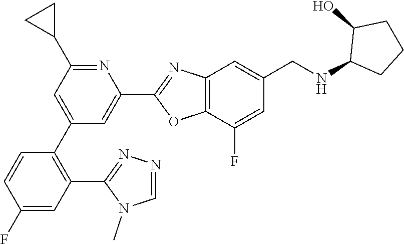 [SMILES: Cn1cnnc1-c1cc(F)ccc1-c1cc(-c2nc3cc(CN[C@@H]4CCC[C@@H]4O)cc(F)c3o2)nc(C2CC2)c1] |
| 433 | AFJ-1 | 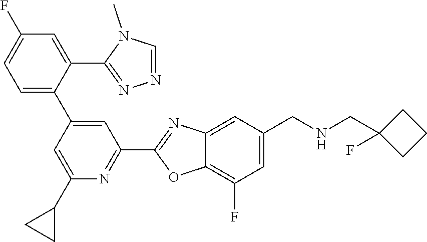 [SMILES: Cn1cnnc1-c1cc(F)ccc1-c1cc(-c2nc3cc(CNCC4(F)CCC4)cc(F)c3o2)nc(C2CC2)c1] |
| 434 | ID-3 | 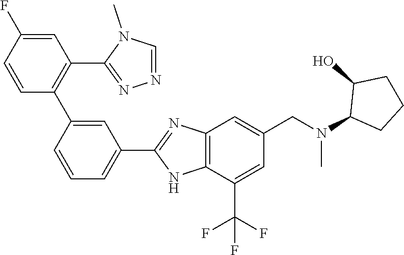 [SMILES: CN(Cc1cc(C(F)(F)F)c2[nH]c(-c3cccc(-c4ccc(F)cc4-c4nncn4C)c3)nc2c1)[C@@H]1CCC[C@@H]1O] |
| 435 | IE-1 | 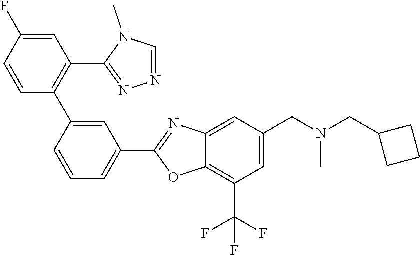 [SMILES: CN(Cc1cc(C(F)(F)F)c2oc(-c3cccc(-c4ccc(F)cc4-c4nncn4C)c3)nc2c1)CC1CCC1] |
| 436 | IF-2 | 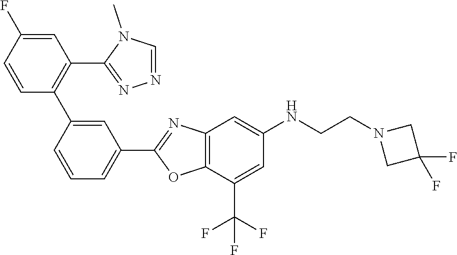 [SMILES: Cn1cnnc1-c1cc(F)ccc1-c1cccc(-c2nc3cc(NCCN4CC(F)(F)C4)cc(C(F)(F)F)c3o2)c1] |
| 437 | IG-6 | 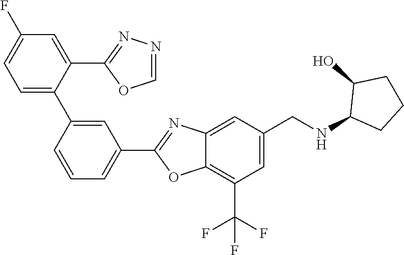 [SMILES: O[C@H]1CCC[C@H]1NCc1cc(C(F)(F)F)c2oc(-c3cccc(-c4ccc(F)cc4-c4nnco4)c3)nc2c1] |
| 438 | AET-1 | 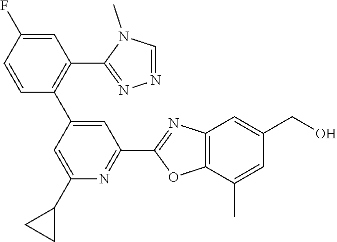 [SMILES: Cc1cc(CO)cc2nc(-c3cc(-c4ccc(F)cc4-c4nncn4C)cc(C4CC4)n3)oc12] |
| 439 | AES-3 | 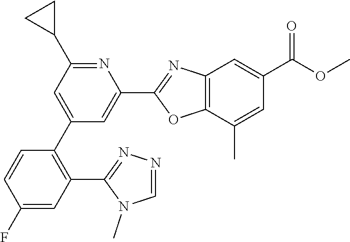 [SMILES: COC(=O)c1cc(C)c2oc(-c3cc(-c4ccc(F)cc4-c4nncn4C)cc(C4CC4)n3)nc2c1] |
| 440 | IA-1 | 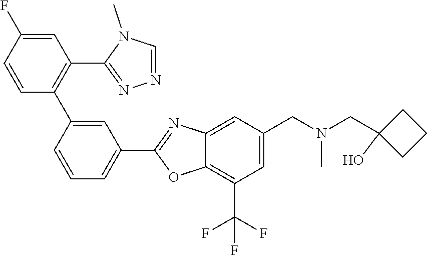 [SMILES: CN(Cc1cc(C(F)(F)F)c2oc(-c3cccc(-c4ccc(F)cc4-c4nncn4C)c3)nc2c1)CC1(O)CCC1] |
| 441 | IB-7 | 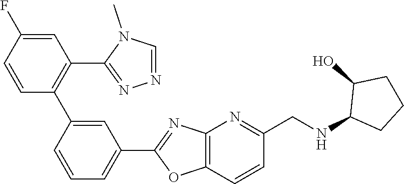 [SMILES: Cn1cnnc1-c1cc(F)ccc1-c1cccc(-c2nc3nc(CN[C@@H]4CCC[C@@H]4O)ccc3o2)c1] |
| 442 | IC-1 | 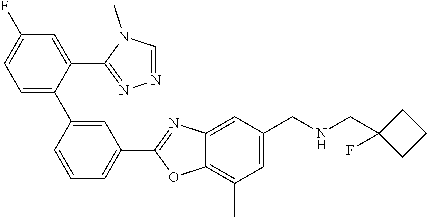 [SMILES: Cc1cc(CNCC2(F)CCC2)cc2nc(-c3cccc(-c4ccc(F)cc4-c4nncn4C)c3)oc12] |
| 443 | AFG-1 | 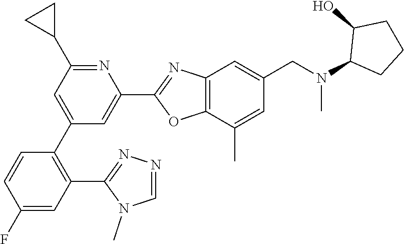 [SMILES: Cc1cc(CN(C)[C@@H]2CCC[C@@H]2O)cc2nc(-c3cc(-c4ccc(F)cc4-c4nncn4C)cc(C4CC4)n3)oc12] |
| 444 | AFH-2 | 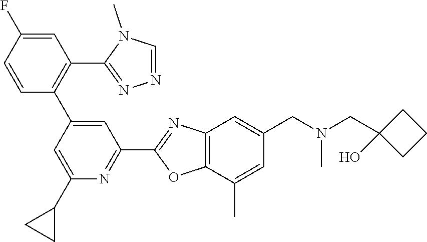 [SMILES: Cc1cc(CN(C)CC2(O)CCC2)cc2nc(-c3cc(-c4ccc(F)cc4-c4nncn4C)cc(C4CC4)n3)oc12] |
| 445 | HV-3 | 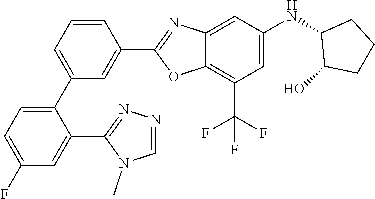 [SMILES: Cn1cnnc1-c1cc(F)ccc1-c1cccc(-c2nc3cc(N[C@@H]4CCC[C@@H]4O)cc(C(F)(F)F)c3o2)c1] |
| 446 | HW-2 | 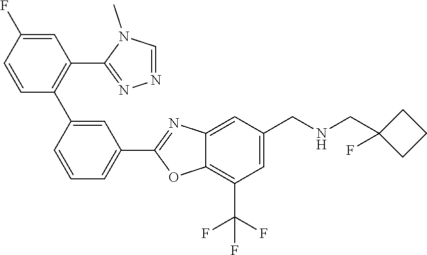 [SMILES: Cn1cnnc1-c1cc(F)ccc1-c1cccc(-c2nc3cc(CNCC4(F)CCC4)cc(C(F)(F)F)c3o2)c1] |
| 447 | HX-8 | 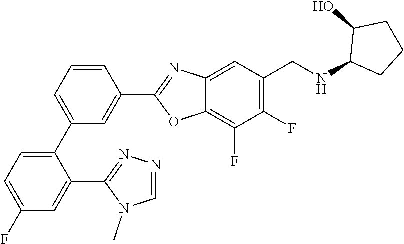 [SMILES: Cn1cnnc1-c1cc(F)ccc1-c1cccc(-c2nc3cc(CN[C@@H]4CCC[C@@H]4O)c(F)c(F)c3o2)c1] |
| 448 | HY-3 | 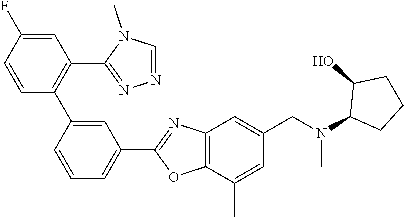 [SMILES: Cc1cc(CN(C)[C@@H]2CCC[C@@H]2O)cc2nc(-c3cccc(-c4ccc(F)cc4-c4nncn4C)c3)oc12] |
| 449 | HZ-2 | 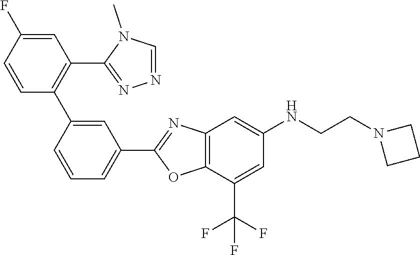 [SMILES: Cn1cnnc1-c1cc(F)ccc1-c1cccc(-c2nc3cc(NCCN4CCC4)cc(C(F)(F)F)c3o2)c1] |
| 450 | AEV-1 | 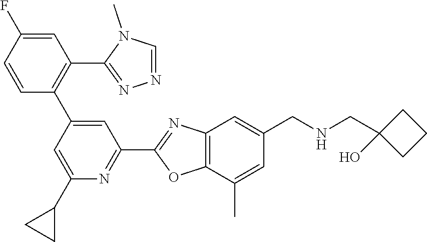 [SMILES: Cc1cc(CNCC2(O)CCC2)cc2nc(-c3cc(-c4ccc(F)cc4-c4nncn4C)cc(C4CC4)n3)oc12] |
| 451 | AEW-1 | 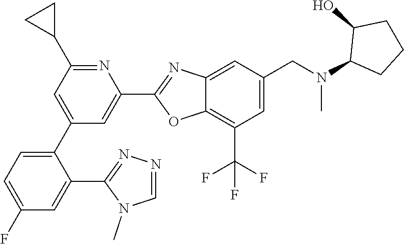 [SMILES: CN(Cc1cc(C(F)(F)F)c2oc(-c3cc(-c4ccc(F)cc4-c4nncn4C)cc(C4CC4)n3)nc2c1)[C@@H]1CCC[C@@H]1O] |
| 452 | HT-3 | 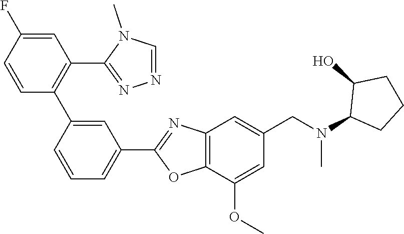 [SMILES: COc1cc(CN(C)[C@@H]2CCC[C@@H]2O)cc2nc(-c3cccc(-c4ccc(F)cc4-c4nncn4C)c3)oc12] |
| 453 | HU-1 | 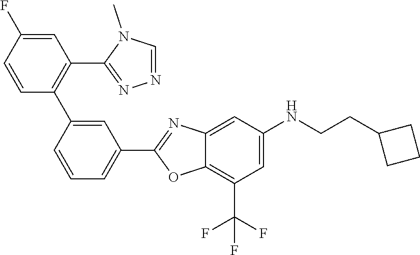 [SMILES: Cn1cnnc1-c1cc(F)ccc1-c1cccc(-c2nc3cc(NCCC4CCC4)cc(C(F)(F)F)c3o2)c1] |
| 454 | HM-3 | 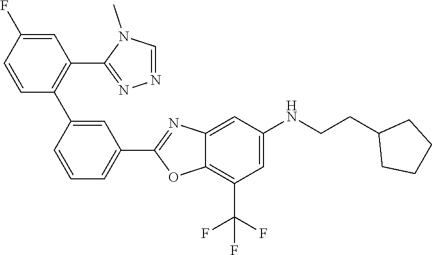 [SMILES: Cn1cnnc1-c1cc(F)ccc1-c1cccc(-c2nc3cc(NCCC4CCCC4)cc(C(F)(F)F)c3o2)c1] |
| 455 | HN-1 | 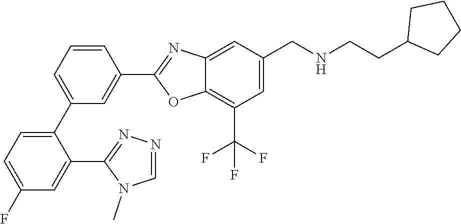 [SMILES: Cn1cnnc1-c1cc(F)ccc1-c1cccc(-c2nc3cc(CNCCC4CCCC4)cc(C(F)(F)F)c3o2)c1] |
| 456 | HO-2 | 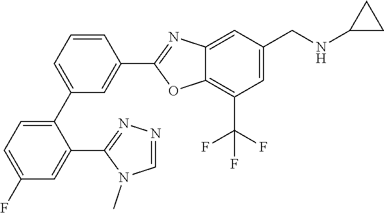 [SMILES: Cn1cnnc1-c1cc(F)ccc1-c1cccc(-c2nc3cc(CNC4CC4)cc(C(F)(F)F)c3o2)c1] |
| 457 | HP-2 | 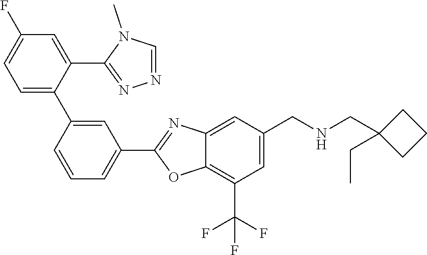 [SMILES: CCC1(CNCc2cc(C(F)(F)F)c3oc(-c4cccc(-c5ccc(F)cc5-c5nncn5C)c4)nc3c2)CCC1] |
| 458 | HQ-2 | 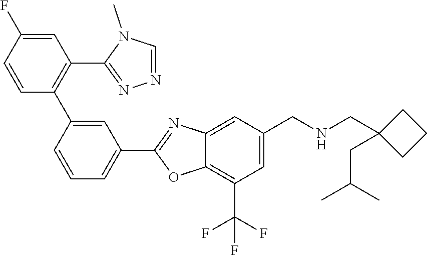 [SMILES: CC(C)CC1(CNCc2cc(C(F)(F)F)c3oc(-c4cccc(-c5ccc(F)cc5-c5nncn5C)c4)nc3c2)CCC1] |
| 459 | HR-2 | 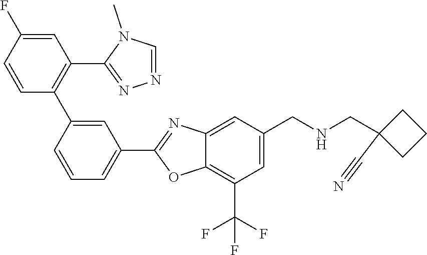 [SMILES: Cn1cnnc1-c1cc(F)ccc1-c1cccc(-c2nc3cc(CNCC4(C#N)CCC4)cc(C(F)(F)F)c3o2)c1] |
| 460 | HS-3 | 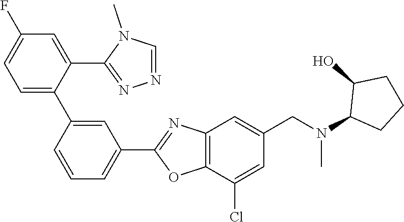 [SMILES: CN(Cc1cc(Cl)c2oc(-c3cccc(-c4ccc(F)cc4-c4nncn4C)c3)nc2c1)[C@@H]1CCC[C@@H]1O] |
| 461 | HG-7 | 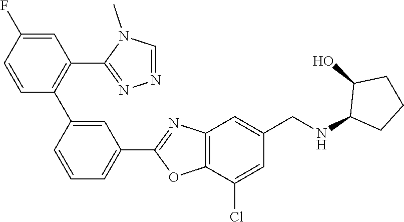 [SMILES: Cn1cnnc1-c1cc(F)ccc1-c1cccc(-c2nc3cc(CN[C@@H]4CCC[C@@H]4O)cc(Cl)c3o2)c1] |
| 462 | HH-3 | 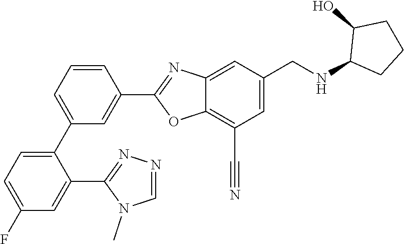 [SMILES: Cn1cnnc1-c1cc(F)ccc1-c1cccc(-c2nc3cc(CN[C@@H]4CCC[C@@H]4O)cc(C#N)c3o2)c1] |
| 463 | HI-2 | 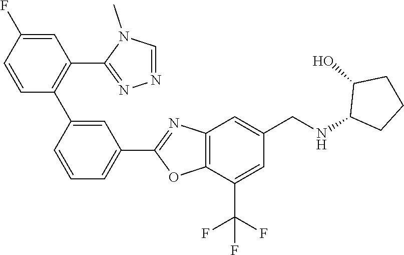 [SMILES: Cn1cnnc1-c1cc(F)ccc1-c1cccc(-c2nc3cc(CN[C@H]4CCC[C@H]4O)cc(C(F)(F)F)c3o2)c1] |
| 464 | HJ-1 | 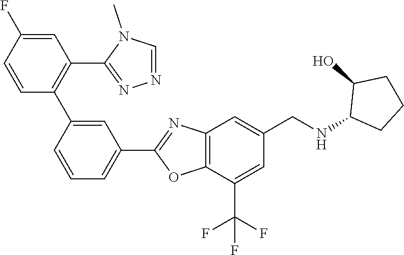 [SMILES: Cn1cnnc1-c1cc(F)ccc1-c1cccc(-c2nc3cc(CN[C@H]4CCC[C@@H]4O)cc(C(F)(F)F)c3o2)c1] |
| 465 | HK-2 | 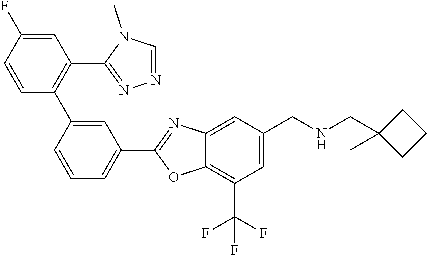 [SMILES: Cn1cnnc1-c1cc(F)ccc1-c1cccc(-c2nc3cc(CNCC4(C)CCC4)cc(C(F)(F)F)c3o2)c1] |
| 466 | HL-2 | 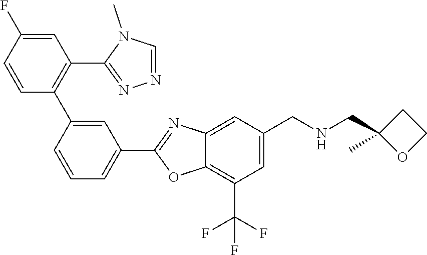 [SMILES: Cn1cnnc1-c1cc(F)ccc1-c1cccc(-c2nc3cc(CNC[C@@]4(C)CCO4)cc(C(F)(F)F)c3o2)c1] |
| 467 | AER-8 | 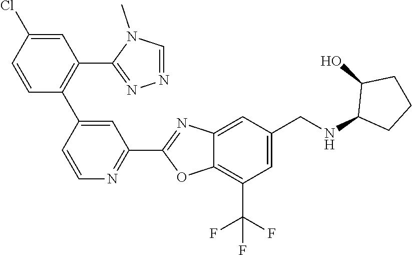 [SMILES: Cn1cnnc1-c1cc(Cl)ccc1-c1ccnc(-c2nc3cc(CN[C@@H]4CCC[C@@H]4O)cc(C(F)(F)F)c3o2)c1] |
| 468 | AFD-1 | 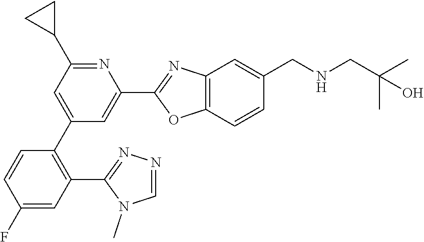 [SMILES: Cn1cnnc1-c1cc(F)ccc1-c1cc(-c2nc3cc(CNCC(C)(C)O)ccc3o2)nc(C2CC2)c1] |
| 469 | AFE-1 | 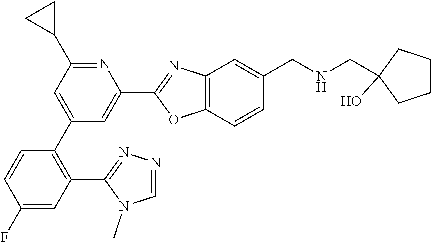 [SMILES: Cn1cnnc1-c1cc(F)ccc1-c1cc(-c2nc3cc(CNCC4(O)CCCC4)ccc3o2)nc(C2CC2)c1] |
| 470 | AEQ-2 | 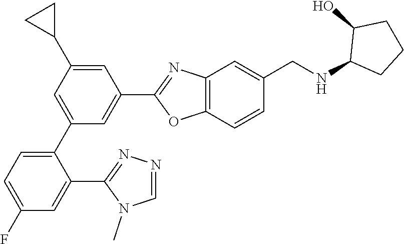 [SMILES: Cn1cnnc1-c1cc(F)ccc1-c1cc(-c2nc3cc(CN[C@@H]4CCC[C@@H]4O)ccc3o2)cc(C2CC2)c1] |
| 471 | AFC-1 | 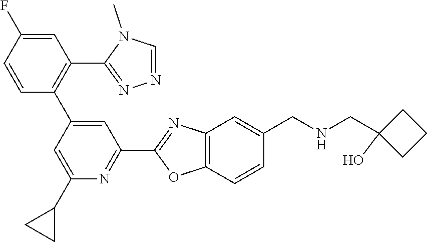 [SMILES: Cn1cnnc1-c1cc(F)ccc1-c1cc(-c2nc3cc(CNCC4(O)CCC4)ccc3o2)nc(C2CC2)c1] |
| 472 | AEP-2 | 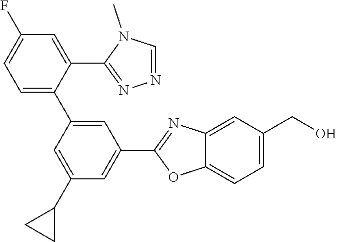 [SMILES: Cn1cnnc1-c1cc(F)ccc1-c1cc(-c2nc3cc(CO)ccc3o2)cc(C2CC2)c1] |
| 473 | AEN-15 | 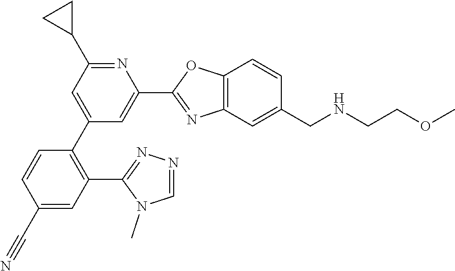 [SMILES: COCCNCc1ccc2oc(-c3cc(-c4ccc(C#N)cc4-c4nncn4C)cc(C4CC4)n3)nc2c1] |
| 474 | JA-1 | 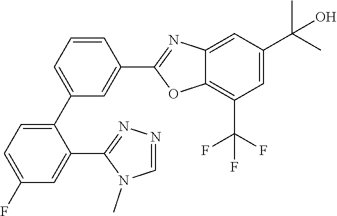 [SMILES: Cn1cnnc1-c1cc(F)ccc1-c1cccc(-c2nc3cc(C(C)(C)O)cc(C(F)(F)F)c3o2)c1] |
| 475 | AEY-2 | 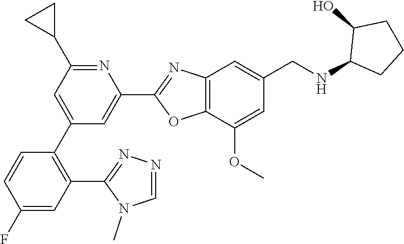 [SMILES: COc1cc(CN[C@@H]2CCC[C@@H]2O)cc2nc(-c3cc(-c4ccc(F)cc4-c4nncn4C)cc(C4CC4)n3)oc12] |
| 476 | AEM-1 | 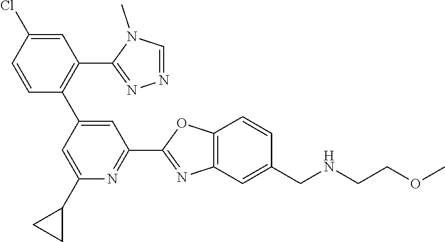 [SMILES: COCCNCc1ccc2oc(-c3cc(-c4ccc(Cl)cc4-c4nncn4C)cc(C4CC4)n3)nc2c1] |
| 477 | AEX-2 | 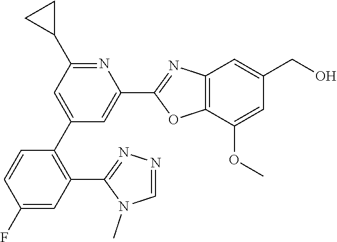 [SMILES: COc1cc(CO)cc2nc(-c3cc(-c4ccc(F)cc4-c4nncn4C)cc(C4CC4)n3)oc12] |
| 478 | AHN-3 | 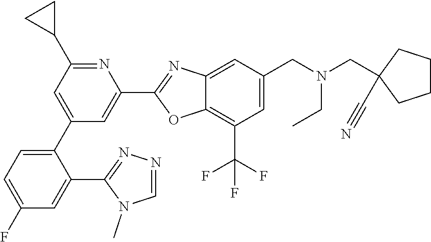 [SMILES: CCN(Cc1cc(C(F)(F)F)c2oc(-c3cc(-c4ccc(F)cc4-c4nncn4C)cc(C4CC4)n3)nc2c1)CC1(C#N)CCCC1] |

In some embodiments, the present invention provides a compound set forth in Table 1, above, or a pharmaceutically acceptable salt thereof. In some embodiments, the present invention provides a compound set forth in Table 1, above.

In some embodiments, the present invention provides a pharmaceutical composition comprising a compound disclosed herein (described in embodiments herein, both singly and in combination), or a pharmaceutically acceptable salt thereof, together with a pharmaceutically acceptable carrier, excipient, or diluent. For example, in some embodiments, the present invention provides a pharmaceutical composition comprising a compound of formula I as defined above, or a pharmaceutically acceptable salt thereof, together with a pharmaceutically acceptable carrier, excipient, or diluent. In some embodiments, the present invention provides a pharmaceutical composition comprising a compound of formula I as defined above, together with a pharmaceutically acceptable carrier, excipient, or diluent. In some embodiments, the present invention provides a pharmaceutical composition comprising a compound set forth in Table 1 above, or a pharmaceutically acceptable salt thereof, together with a pharmaceutically acceptable carrier, excipient, or diluent. In some embodiments, the present invention provides a pharmaceutical composition comprising a compound set forth in Table 1 above, together with a pharmaceutically acceptable carrier, excipient, or diluent.

In some embodiments, the present invention provides a compound of formula I as defined above, or a pharmaceutically acceptable salt thereof, or a pharmaceutical composition comprising a compound of formula I as defined above, or a pharmaceutically acceptable salt thereof, and a pharmaceutically acceptable carrier, adjuvant, or vehicle for use as a medicament.

In some embodiments, the invention also provides a compound described herein (such as a compound of formula I), or pharmaceutical compositions described herein, for use in a method for inhibiting Cbl-b as described herein and/or in a method for treating a Cbl-b-dependent disorder as described herein. In some embodiments, the invention also provides a compound described herein (such as a compound of formula I), or pharmaceutical compositions described herein, for use in a method for inhibiting Cbl-b as described herein. In some embodiments, the invention also provides a compound described herein (such as a compound of formula I), or pharmaceutical compositions described herein, for use in a method for treating a Cbl-b-dependent disorder as described herein.

In some embodiments, the invention also provides a compound described herein (such as a compound of formula I), or pharmaceutical compositions described herein, for use in a method for modulating Cbl-b as described herein and/or in a method for treating a Cbl-b-dependent disorder as described herein. In some embodiments, the invention also provides a compound described herein (such as a compound of formula I), or pharmaceutical compositions described herein, for use in a method for modulating Cbl-b as described herein. In some embodiments, the invention also provides a compound described herein (such as a compound of formula I), or pharmaceutical compositions described herein, for use in a method for treating a Cbl-b-dependent disorder as described herein.

### 4. General Methods of Providing the Present Compounds

The compounds of this invention may be prepared or isolated in general by synthetic and/or semi-synthetic methods known to those skilled in the art for analogous compounds and by methods described in detail in the Examples, herein.

### 5. Uses, Formulation and Administration

### Pharmaceutically Acceptable Compositions

According to another embodiment, the invention provides a composition comprising a compound of this invention or a pharmaceutically acceptable derivative thereof and a pharmaceutically acceptable carrier, adjuvant, or vehicle. In certain embodiments, the amount of compound in compositions of this invention is such that is effective to measurably inhibit Cbl-b, or a mutant thereof, in a biological sample or in a patient. In certain embodiments, a composition of this invention is formulated for administration to a patient in need of such composition. In some embodiments, a composition of this invention is formulated for oral administration to a patient.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily active metabolite or residue thereof.

As used herein, the term “active metabolite or residue thereof” means that a metabolite or residue thereof is also an inhibitor of Cbl-b, or a mutant thereof.

The subject matter disclosed herein includes prodrugs, metabolites, derivatives, and pharmaceutically acceptable salts of compounds of the invention. Metabolites include compounds produced by a process comprising contacting a compound of the invention with a mammal for a period of time sufficient to yield a metabolic product thereof. If the compound of the invention is a base, the desired pharmaceutically acceptable salt may be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like. If the compound of the invention is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

A compound of the invention can be in the form of a “prodrug,” which includes compounds with moieties which can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Prodrugs which are converted to active forms through other mechanisms in vivo are also included. In aspects, the compounds of the invention are prodrugs of any of the formulae herein.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Alternatively, pharmaceutically acceptable compositions of this invention may be administered in the form of suppositories for rectal or vaginal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

The amount of compounds of the present invention that may be combined with the carrier materials to produce a composition in a single dosage form will vary depending upon the host treated, the particular mode of administration. Preferably, provided compositions should be formulated so that a dosage of between 0.01 and 100 mg/kg, 0.01 and 50 mg/kg, or 1 and 25 mg/kg, body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment.

### Uses of Compounds and Pharmaceutically Acceptable Compositions

The compounds and compositions described herein are generally useful for the inhibition of E3 ligase activity of one or more enzymes. In some embodiments the E3 ligase inhibited by the compounds and methods of the invention is Cbl-b.

The presently disclosed compounds find use in inhibiting the enzyme Cbl-b. In one embodiment, the subject matter disclosed herein is directed to a method of inhibiting Cbl-b, the method comprising contacting Cbl-b with an effective amount of a compound of the invention or a pharmaceutical composition described herein.

The presently disclosed compounds can be used in a method for inhibiting Cbl-b. Such methods comprise contacting Cbl-b with an effective amount of a presently disclosed compound. By “contact” is intended bringing the compound within close enough proximity to an isolated Cbl-b enzyme or a cell expressing Cbl-b such that the compound is able to bind to and inhibit the Cbl-b. The compound can be contacted with Cbl-b in vitro or in vivo via administration of the compound to a subject.

In one aspect, provided herein is a method of inhibiting Cbl-b in a biological sample. The method comprises contacting the sample with a compound disclosed herein (such as a compound of formula I), a pharmaceutically acceptable salt thereof, or a pharmaceutical composition disclosed herein (such as a composition comprising a compound disclosed herein [such as a compound of formula I] and a pharmaceutically acceptable carrier, adjuvant, or vehicle). The term “biological sample”, as used herein, includes, without limitation, cell cultures or extracts thereof, biopsied material obtained from a mammal or extracts thereof, and blood, saliva, urine, feces, semen, tears, or other body fluids or extracts thereof.

The present disclosure provides methods of inhibiting Cbl-b in a patient. The method comprises administering to a patient a compound disclosed herein (such as a compound of formula I), a pharmaceutically acceptable salt thereof, or a pharmaceutical composition disclosed herein (such as a composition comprising a compound disclosed herein [such as a compound of formula I] and a pharmaceutically acceptable carrier, adjuvant, or vehicle).

The presently disclosed compounds may or may not be selective Cbl-b inhibitors. A selective Cbl-b inhibitor inhibits the biological activity of Cbl-b by an amount that is statistically greater than the inhibiting effect of the inhibitor on any other protein (e.g., other E3 ligases). In some of these embodiments, the IC₅₀ of the Cbl-b inhibitor for Cbl-b is about 90%, 80%, 70%, 60%, 50%, 40%, 30%, 20%, 10%, 0.1%, 0.01%, 0.001%, or less of the IC₅₀ of the Cbl-b inhibitor for another E3 ligase.

Any method known in the art to measure the ligase activity of Cbl-b may be used to determine if Cbl-b has been inhibited, including in vitro kinase assays, immunoblots with antibodies specific for ubiquitinated targets of Cbl-b, or the measurement of a downstream biological effect of Cbl-b ligase activity.

The presently disclosed compounds can be used to treat an Cbl-b-dependent disorder. As used herein, a “Cbl-b-dependent disorder” is a pathological condition in which Cbl-b activity is necessary for the genesis or maintenance of the pathological condition.

Accordingly, in one aspect, provided herein is a method of treating a Cbl-b-mediated disorder, disease, or condition in a patient. The method comprises administering to said patient a compound disclosed herein (such as a compound of formula I), a pharmaceutically acceptable salt thereof, or a pharmaceutical composition disclosed herein (such as a composition comprising a compound disclosed herein [such as a compound of formula I] and a pharmaceutically acceptable carrier, adjuvant, or vehicle).

Provided herein are compounds and pharmaceutical compositions that inhibit the Cbl-b enzyme, as well as methods of treatment using such compounds and pharmaceutical compositions. The compounds and compositions can be used in methods of modulating the immune system, for treatment of diseases, and for treatment of cells in vivo, in vitro, or ex vivo.

T-cell activation and T-cell tolerance are tightly controlled processes regulating the immune response to tumors while preventing autoimmunity. Tolerance prevents the immune system from attacking cells expressing “self” antigens. During peripheral tolerance, T-cells that recognize “self” antigens (i.e., self-reactive T-cells) become functionally unresponsive or are deleted after encountering “self” antigens outside of the thymus. Peripheral tolerance processes therefore are important for preventing autoimmune diseases. Normally, cancer cells are removed by activated T-cells that recognize tumor antigens expressed on the surface of the cancer cells. However, in cancer, the tumor microenvironment can support T-cell tolerance to cancer cells, which allows cancer cells to avoid recognition and removal by the immune system. The ability of cancer cells to avoid tumor immunosurveillance can contribute to uncontrolled tumor growth. Therefore, T-cell tolerance can be a form of T-cell dysfunction. General principles of T-cell dysfunction are well known in the art (see Schietinger etal, Trends Immunol., 35: 51-60, 2014). Additional types of T-cell dysfunction that can contribute to uncontrolled tumor growth include T-cell exhaustion, T-cell senescence, and/or T-cell anergy. Therefore, treating T-cell dysfunction, for example, by increasing T-cell activation, increasing T-cell proliferation, decreasing T-cell tolerance, and/or decreasing T-cell exhaustion, is beneficial for preventing or treating cancer. Additional cells of the immune system are important for recognition and removal of cancer cells during immune surveillance. For example, Natural Killer (NK)-cells are lymphocytes of the innate immune system that are able to identify and kill cancer cells (see Martinez-Losato et al, Clin Cancer Res., 21: 5048-5056, 2015). Recent studies have also shown that B-cell subsets with distinct phenotypes and functions exhibit diverse roles in the anti -tumor response (see Saravaria et al, Cell Mol Immunol., 14: 662-674, 2017). Due to their role in tumor surveillance, NK-cells and B-cells may also be amenable as therapeutic targets for the prevention or treatment of cancer.

Cbl-b is a RING-type E3 ligase that plays an important role in the immune system due to its function as a negative regulator of immune activation. Cbl-b has an essential role in decreasing the activation of T-cells, thereby enhancing T-cell tolerance. Studies have found that Cbl-b-deficient T-cells display lower thresholds for activation by antigen recognition receptors and co-stimulatory molecules (e.g., CD28). For example, loss of Cbl-b in T-cells uncouples the requirement for CD28 costimulation during T-cell activation and proliferation (see Bachmaier el al, Nature, 403: 211-216, 2000). Such cbl-b−/− T-cells are largely resistant to T-cell anergy, a tolerance mechanism in which T-cells are functionally inactivated and T-cell proliferation is greatly impaired (see Jeon el al, Immunity, 21: 167-177, 2004; and Schwartz et al, Annu Rev Immunol., 21: 305-34, 2003). In support of this, loss of Cbl-b in cbl-b knockout mice resulted in impaired induction of T-cell tolerance and exacerbated autoimmunity (see Jeon el al, Immunity, 21: 167-177, 2004). Importantly, loss of Cbl-b in mice also resulted in a robust anti-tumor response that depends primarily on cytotoxic T-cells. One study showed that cbl-b−/− CD8+ T-cells are resistant to T regulatory cell-mediated suppression and exhibit enhanced activation and tumor infiltration. Therapeutic transfer of naive cbl-b−/− CD8+ T-cells was sufficient to mediate rejection of established tumors (see Loeser et al, J Exp Med., 204: 879-891, 2007). Recent studies have shown that Cbl-b also plays a role in NK-cell activation. Genetic deletion of Cbl-b or targeted inactivation of its E3 ligase activity allowed NK-cells to spontaneously reject metastatic tumors in a mouse model (see Paolino et al, Nature, 507: 508-512, 2014).

Provided herein are compounds and compositions that are potent inhibitors of Cbl-b and can be used in novel approaches to treat diseases such as cancer. In some embodiments, the compounds and compositions provided herein can be used in methods of modulating the immune system, such as increasing activation of T-cells, NK-cells and B-cells, as well as in the treatment of such cells in vivo, in vitro, or ex vivo.

Provided herein are methods for modulating activity of an immune cell (e.g., a T-cell, a B-cell, or a NK-cell) such as by contacting the immune cell with an effective amount of a Cbl-b inhibitor described herein or a composition thereof. Further provided are in vivo methods of modulating a response in an individual in need thereof (e.g., an individual with cancer), wherein the method comprises administration of an effective amount of a Cbl-b inhibitor described herein or a composition thereof.

Additionally, provided are Cbl-b inhibitors for use as therapeutic active substances. A Cbl-b inhibitor for use in treating or preventing a disease or condition associated with Cbl-b activity is provided. Also, a Cbl-b inhibitor for use in treating cancer is provided. Further provided is the use of a Cbl-b inhibitor in the manufacture of a medicament for treating or preventing a disease or condition associated with Cbl-b activity. Also provided is the use of a Cbl-b inhibitor in the manufacture of a medicament for treating cancer.

Moreover, this disclosure provides treatment methods, medicaments, and uses comprising a Cbl-b inhibitor as part of a combination therapy for treating cancer involving one or more of an immune checkpoint inhibitor, an antineoplastic agent, and radiation therapy.

In some embodiments of the treatment methods, medicaments, and uses of this disclosure, the cancer is a hematologic cancer such as lymphoma, a leukemia, or a myeloma. In other embodiments of the treatment methods, medicaments, and uses of this disclosure, the cancer is a non-hematologic cancer such as a sarcoma, a carcinoma, or a melanoma.

Hematologic cancers include, but are not limited to, one or more leukemias such as B-cell acute lymphoid leukemia (“BALL”), T-cell acute lymphoid leukemia (“TALL”), acute lymphoid leukemia (ALL); one or more chronic leukemias including, but not limited to, chronic myelogenous leukemia (CML) and chronic lymphocytic leukemia (CLL); additional hematologic cancers or hematologic conditions including, but not limited to, B-cell prolymphocytic leukemia, blastic plasmacytoid dendritic cell neoplasm, Burkitf s lymphoma, diffuse large B-cell lymphoma, follicular lymphoma, hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin's lymphoma, plasmablastic lymphoma, plasmacytoid dendritic cell neoplasm, Waldenstrom macroglobulinemia, and“preleukemia,” which are a diverse collection of hematological conditions united by ineffective production (or dysplasia) of myeloid blood cells.

Non-hematologic cancers include, but are not limited to, a neuroblastoma, renal cell carcinoma, colon cancer, colorectal cancer, breast cancer, epithelial squamous cell cancer, melanoma, stomach cancer, brain cancer, lung cancer (e.g., NSCLC), pancreatic cancer, cervical cancer, ovarian cancer, liver cancer, bladder cancer, prostate cancer, testicular cancer, thyroid cancer, uterine cancer, adrenal cancer, and head and neck cancer.

In some aspects, the effectiveness of administration of a Cbl-b inhibitor in the treatment of a disease or disorder such as cancer is measured by assessing clinical outcome, such as reduction in tumor size or number of tumors, and/or survival. In certain embodiments, “treating cancer” comprises assessing a patient's response to the treatment regimen according to the Response Evaluation Criteria in Solid Tumors (RECIST version 1.1) as described (see, e.g., Eisenhauer et al, Eur J Cancer, 45:228-247, 2009; and Nishino et al., Am J Roentgenol, 195: 281-289, 2010). Response criteria to determine objective anti -tumor responses per RECIST 1.1 include complete response (CR); partial response (PR); progressive disease (PD); and stable disease (SD).

Accordingly, in some embodiments, the Cbl-b-mediated disorder is a hematologic cancer. In one aspect, provided herein is a method of treating a hematologic cancer in a patient. The method comprises administering to said patient a compound disclosed herein (such as a compound of formula I), a pharmaceutically acceptable salt thereof, or a pharmaceutical composition disclosed herein (such as a composition comprising a compound disclosed herein [such as a compound of formula I] and a pharmaceutically acceptable carrier, adjuvant, or vehicle).

More generally, in some embodiments, the Cbl-b-mediated disorder is a non-hematologic cancer. In one aspect, provided herein is a method of treating a non-hematologic cancer in a patient. The method comprises administering to said patient a compound disclosed herein (such as a compound of formula I), a pharmaceutically acceptable salt thereof, or a pharmaceutical composition disclosed herein (such as a composition comprising a compound disclosed herein [such as a compound of formula I] and a pharmaceutically acceptable carrier, adjuvant, or vehicle). In some embodiments, the non-hematologic cancer is a neuroblastoma, renal cell carcinoma, colon cancer, colorectal cancer, breast cancer, epithelial squamous cell cancer, melanoma, stomach cancer, brain cancer, lung cancer (e.g., NSCLC), pancreatic cancer, cervical cancer, ovarian cancer, liver cancer, bladder cancer, prostate cancer, testicular cancer, thyroid cancer, uterine cancer, adrenal cancer, and head and neck cancer. In some embodiments, the non-hematologic cancer is colon cancer. In some embodiments, the non-hematologic cancer is liver cancer. In some embodiments, the non-hematologic cancer is lung cancer. In some embodiments, the non-hematologic cancer is breast cancer. In some embodiments, the non-hematologic cancer is brain cancer.

It has also been reported that Cbl-b inhibitors may provide benefit to patients suffering from cancer. Accordingly, in some embodiments, the Cbl-b-mediated disorder is a cancer. In one aspect, provided herein is a method of treating a cancer in a patient. The method comprises administering to said patient a compound disclosed herein (such as a compound of formula I), a pharmaceutically acceptable salt thereof, or a pharmaceutical composition disclosed herein (such as a composition comprising a compound disclosed herein [such as a compound of formula I] and a pharmaceutically acceptable carrier, adjuvant, or vehicle).

Examples of cancers that are treatable using the compounds of the present disclosure include, but are not limited to, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, endometrial cancer, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T -cell lymphoma, environmentally induced cancers including those induced by asbestos, and combinations of said cancers.

In some embodiments, cancers that are treatable using the compounds of the present disclosure include, but are not limited to, solid tumors (e.g., prostate cancer, colon cancer, esophageal cancer, endometrial cancer, ovarian cancer, uterine cancer, renal cancer, hepatic cancer, pancreatic cancer, gastric cancer, breast cancer, lung cancer, cancers of the head and neck, thyroid cancer, glioblastoma, sarcoma, bladder cancer, etc.), hematological cancers (e.g., lymphoma, leukemia such as acute lymphoblastic leukemia (ALL), acute myelogenous leukemia (AML), chronic lymphocytic leukemia (CLL), chronic myelogenous leukemia (CML), DLBCL, mantle cell lymphoma, Non-Hodgkin lymphoma (including relapsed or refractory NHL and recurrent follicular), Hodgkin lymphoma or multiple myeloma) and combinations of said cancers.

In certain embodiments, the cancer is brain cancer, leukemia, skin cancer, prostate cancer, thyroid cancer, colon cancer, lung cancer or sarcoma. In another embodiment the cancer is selected from the group consisting of glioma, glioblastoma multiforme, paraganglioma, suprantentorial primordial neuroectodermal tumors, acute myeloid leukemia, myelodysplastic syndrome, chronic myelogenous leukemia, melanoma, breast, prostate, thyroid, colon, lung, central chondrosarcoma, central and periosteal chondroma tumors, fibrosarcoma, and cholangiocarcinoma.

In certain embodiments, the cancer is selected from brain and spinal cancers, cancers of the head and neck, leukemia and cancers of the blood, skin cancers, cancers of the reproductive system, cancers of the gastrointestinal system, liver and bile duct cancers, kidney and bladder cancers, bone cancers, lung cancers, malignant mesothelioma, sarcomas, lymphomas, glandular cancers, thyroid cancers, heart tumors, germ cell tumors, malignant neuroendocrine (carcinoid) tumors, midline tract cancers, and cancers of unknown primary (cancers in which a metastasized cancer is found but the original cancer site is not known). In particular embodiments, the cancer is present in an adult patient; in additional embodiments, the cancer is present in a pediatric patient. In particular embodiments, the cancer is AIDS-related.

In a further embodiment, the cancer is selected from brain and spinal cancers. In particular embodiments, the cancer is selected from the group consisting of anaplastic astrocytomas, glioblastomas, astrocytomas, and estheosioneuroblastomas (olfactory blastomas). In particular embodiments, the brain cancer is selected from the group consisting of astrocytic tumor (e.g., pilocytic astrocytoma, subependymal giant-cell astrocytoma, diffuse astrocytoma, pleomorphic xanthoastrocytoma, anaplastic astrocytoma, astrocytoma, giant cell glioblastoma, glioblastoma, secondary glioblastoma, primary adult glioblastoma, and primary pediatric glioblastoma), oligodendroglial tumor (e.g., oligodendroglioma, and anaplastic oligodendroglioma), oligoastrocytic tumor (e.g., oligoastrocytoma, and anaplastic oligoastrocytoma), ependymoma (e.g., myxopapillary ependymoma, and anaplastic ependymoma); medulloblastoma, primitive neuroectodermal tumor, schwannoma, meningioma, atypical meningioma, anaplastic meningioma, pituitary adenoma, brain stem glioma, cerebellar astrocytoma, cerebral astorcytoma/malignant glioma, visual pathway and hypothalmic glioma, and primary central nervous system lymphoma. In specific instances of these embodiments, the brain cancer is selected from the group consisting of glioma, glioblastoma multiforme, paraganglioma, and suprantentorial primordial neuroectodermal tumors (sPNET).

In specific embodiments, the cancer is selected from cancers of the head and neck, including nasopharyngeal cancers, nasal cavity and paranasal sinus cancers, hypopharyngeal cancers, oral cavity cancers (e.g., squamous cell carcinomas, lymphomas, and sarcomas), lip cancers, oropharyngeal cancers, salivary gland tumors, cancers of the larynx (e.g., laryngeal squamous cell carcinomas, rhabdomyosarcomas), and cancers of the eye or ocular cancers. In particular embodiments, the ocular cancer is selected from the group consisting of intraocular melanoma and retinoblastoma.

In specific embodiments, the cancer is selected from leukemia and cancers of the blood. In particular embodiments, the cancer is selected from the group consisting of myeloproliferative neoplasms, myelodysplastic syndromes, myelodysplastic/myeloproliferative neoplasms, acute myeloid leukemia (AML), myelodysplastic syndrome (MDS), chronic myelogenous leukemia (CML), myeloproliferative neoplasm (MPN), post-MPN AML, post-MDS AML, del(5q)-associated high risk MDS or AML, blast-phase chronic myelogenous leukemia, angioimmunoblastic lymphoma, acute lymphoblastic leukemia, Langerans cell histiocytosis, hairy cell leukemia, and plasma cell neoplasms including plasmacytomas and multiple myelomas. Leukemias referenced herein may be acute or chronic.

In specific embodiments, the cancer is selected from skin cancers. In particular embodiments, the skin cancer is selected from the group consisting of melanoma, squamous cell cancers, and basal cell cancers.

In specific embodiments, the cancer is selected from cancers of the reproductive system. In particular embodiments, the cancer is selected from the group consisting of breast cancers, cervical cancers, vaginal cancers, ovarian cancers, prostate cancers, penile cancers, and testicular cancers. In specific instances of these embodiments, the cancer is a breast cancer selected from the group consisting of ductal carcinomas and phyllodes tumors. In specific instances of these embodiments, the breast cancer may be male breast cancer or female breast cancer. In specific instances of these embodiments, the cancer is a cervical cancer selected from the group consisting of squamous cell carcinomas and adenocarcinomas. In specific instances of these embodiments, the cancer is an ovarian cancer selected from the group consisting of epithelial cancers.

In specific embodiments, the cancer is selected from cancers of the gastrointestinal system. In particular embodiments, the cancer is selected from the group consisting of esophageal cancers, gastric cancers (also known as stomach cancers), gastrointestinal carcinoid tumors, pancreatic cancers, gallbladder cancers, colorectal cancers, and anal cancer. In instances of these embodiments, the cancer is selected from the group consisting of esophageal squamous cell carcinomas, esophageal adenocarcinomas, gastric adenocarcinomas, gastrointestinal carcinoid tumors, gastrointestinal stromal tumors, gastric lymphomas, gastrointestinal lymphomas, solid pseudopapillary tumors of the pancreas, pancreatoblastoma, islet cell tumors, pancreatic carcinomas including acinar cell carcinomas and ductal adenocarcinomas, gallbladder adenocarcinomas, colorectal adenocarcinomas, and anal squamous cell carcinomas.

In specific embodiments, the cancer is selected from liver and bile duct cancers. In particular embodiments, the cancer is liver cancer (hepatocellular carcinoma). In particular embodiments, the cancer is bile duct cancer (cholangiocarcinoma); in instances of these embodiments, the bile duct cancer is selected from the group consisting of intrahepatic cholangiocarcinoma and extrahepatic cholangiocarcinoma.

In specific embodiments, the cancer is selected from kidney and bladder cancers. In particular embodiments, the cancer is a kidney cancer selected from the group consisting of renal cell cancer, Wilms tumors, and transitional cell cancers. In particular embodiments, the cancer is a bladder cancer selected from the group consisting of urethelial carcinoma (a transitional cell carcinoma), squamous cell carcinomas, and adenocarcinomas.

In specific embodiments, the cancer is selected from bone cancers. In particular embodiments, the bone cancer is selected from the group consisting of osteosarcoma, malignant fibrous histiocytoma of bone, Ewing sarcoma, and chordoma.

In specific embodiments, the cancer is selected from lung cancers. In particular embodiments, the lung cancer is selected from the group consisting of non-small cell lung cancer, small cell lung cancers, bronchial tumors, and pleuropulmonary blastomas.

In specific embodiments, the cancer is selected from malignant mesothelioma. In particular embodiments, the cancer is selected from the group consisting of epithelial mesothelioma and sarcomatoids.

In specific embodiments, the cancer is selected from sarcomas. In particular embodiments, the sarcoma is selected from the group consisting of central chondrosarcoma, central and periosteal chondroma, fibrosarcoma, clear cell sarcoma of tendon sheaths, and Kaposi's sarcoma.

In specific embodiments, the cancer is selected from lymphomas. In particular embodiments, the cancer is selected from the group consisting of Hodgkin lymphoma (e.g., Reed-Sternberg cells), non-Hodgkin lymphoma (e.g., diffuse large B-cell lymphoma, follicular lymphoma, mycosis fungoides, Sezary syndrome, primary central nervous system lymphoma), cutaneous T-cell lymphomas, and primary central nervous system lymphomas.

In specific embodiments, the cancer is selected from glandular cancers. In particular embodiments, the cancer is selected from the group consisting of adrenocortical cancer, pheochromocytomas, paragangliomas, pituitary tumors, thymoma, and thymic carcinomas.

In specific embodiments, the cancer is selected from thyroid cancers. In particular embodiments, the thyroid cancer is selected from the group consisting of medullary thyroid carcinomas, papillary thyroid carcinomas, and follicular thyroid carcinomas.

In specific embodiments, the cancer is selected from germ cell tumors. In particular embodiments, the cancer is selected from the group consisting of malignant extracranial germ cell tumors and malignant extragonadal germ cell tumors. In specific instances of these embodiments, the malignant extragonadal germ cell tumors are selected from the group consisting of nonseminomas and seminomas.

In specific embodiments, the cancer is selected from heart tumors. In particular embodiments, the heart tumor is selected from the group consisting of malignant teratoma, lymphoma, rhabdomyosacroma, angiosarcoma, chondrosarcoma, infantile fibrosarcoma, and synovial sarcoma.

In some embodiments, cancers treatable with compounds of the present disclosure include melanoma (e.g., metastatic malignant melanoma), renal cancer (e.g. clear cell carcinoma), prostate cancer (e.g. hormone refractory prostate adenocarcinoma), breast cancer, triple-negative breast cancer, colon cancer and lung cancer (e.g. non-small cell lung cancer and small cell lung cancer). Additionally, the disclosure includes refractory or recurrent malignancies whose growth may be inhibited using the compounds of the disclosure.

In some embodiments, diseases and indications that are treatable using the compounds of the present disclosure include, but are not limited to hematological cancers, sarcomas, lung cancers, gastrointestinal cancers, genitourinary tract cancers, liver cancers, bone cancers, nervous system cancers, gynecological cancers, and skin cancers.

Exemplary hematological cancers include lymphomas and leukemias such as acute lymphoblastic leukemia (ALL), acute myelogenous leukemia (AML), acute promyelocytic leukemia (APL), chronic lymphocytic leukemia (CLL), chronic myelogenous leukemia (CML), diffuse large B-cell lymphoma (DLBCL), mantle cell lymphoma, Non-Hodgkin lymphoma (including relapsed or refractory NHL and recurrent follicular), Hodgkin lymphoma, myeloproliferative diseases (e.g., primary myelofibrosis (PMF), polycythemia vera (PV), essential thrombocytosis (ET)), myelodysplasia syndrome (MDS), T-cell acute lymphoblastic lymphoma (T-ALL), multiple myeloma, cutaneous T-cell lymphoma, Waldenstrom's Macroglubulinemia, hairy cell lymphoma, chronic myelogenic lymphoma and Burkitt's lymphoma.

Exemplary sarcomas include chondrosarcoma, Ewing's sarcoma, osteosarcoma, rhabdomyosarcoma, angiosarcoma, fibrosarcoma, liposarcoma, myxoma, rhabdomyoma, rhabdosarcoma, fibroma, lipoma, harmatoma, and teratoma.

Exemplary lung cancers include non-small cell lung cancer (NSCLC), small cell lung cancer, bronchogenic carcinoma (squamous cell, undifferentiated small cell, undifferentiated large cell, adenocarcinoma), alveolar (bronchiolar) carcinoma, bronchial adenoma, chondromatous hamartoma, and mesothelioma.

Exemplary gastrointestinal cancers include cancers of the esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma), and colorectal cancer.

Exemplary genitourinary tract cancers include cancers of the kidney (adenocarcinoma, Wilm's tumor [nephroblastoma]), bladder and urethra (squamous cell carcinoma, transitional cell carcinoma, adenocarcinoma), prostate (adenocarcinoma, sarcoma), and testis (seminoma, teratoma, embryonal carcinoma, teratocarcinoma, choriocarcinoma, sarcoma, interstitial cell carcinoma, fibroma, fibroadenoma, adenomatoid tumors, lipoma).

Exemplary liver cancers include hepatoma (hepatocellular carcinoma), cholangiocarcinoma, hepatoblastoma, angiosarcoma, hepatocellular adenoma, and hemangioma.

Exemplary bone cancers include, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors

Exemplary nervous system cancers include cancers of the skull (osteoma, hemangioma, granuloma, xanthoma, osteitis deformans), meninges (meningioma, meningiosarcoma, gliomatosis), brain (astrocytoma, meduoblastoma, glioma, ependymoma, germinoma (pinealoma), glioblastoma, glioblastoma multiform, oligodendroglioma, schwannoma, retinoblastoma, congenital tumors), and spinal cord (neurofibroma, meningioma, glioma, sarcoma), as well as neuroblastoma and Lhermitte-Duclos disease.

Exemplary gynecological cancers include cancers of the uterus (endometrial carcinoma), cervix (cervical carcinoma, pre -tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), and fallopian tubes (carcinoma).

Exemplary skin cancers include melanoma, basal cell carcinoma, squamous cell carcinoma, Kaposi's sarcoma, Merkel cell skin cancer, moles dysplastic nevi, lipoma, angioma, dermatofibroma, and keloids. In some embodiments, diseases and indications that are treatable using the compounds of the present disclosure include, but are not limited to, sickle cell disease (e.g., sickle cell anemia), triple-negative breast cancer (TNBC), myelodysplastic syndromes, testicular cancer, bile duct cancer, esophageal cancer, and urothelial carcinoma.

Exemplary head and neck cancers include glioblastoma, melanoma, rhabdosarcoma, lymphosarcoma, osteosarcoma, squamous cell carcinomas, adenocarcinomas, oral cancer, laryngeal cancer, nasopharyngeal cancer, nasal and paranasal cancers, thyroid and parathyroid cancers.

The presently disclosed compounds may be administered in any suitable manner known in the art. In some embodiments, the compound of the invention or a pharmaceutically acceptable salt, prodrug, metabolite, or derivative thereof is administered intravenously, intramuscularly, subcutaneously, topically, orally, transdermally, intraperitoneally, intraorbitally, by implantation, by inhalation, intrathecally, intraventricularly, intratumorally, or intranasally.

In some embodiments, the Cbl-b inhibitor is administered continuously. In other embodiments, the Cbl-b inhibitor is administered intermittently. Moreover, treatment of a subject with an effective amount of a Cbl-b inhibitor can include a single treatment or can include a series of treatments.

It is understood that appropriate doses of the active compound depends upon a number of factors within the knowledge of the ordinarily skilled physician or veterinarian. The dose(s) of the active compound will vary, for example, depending upon the age, body weight, general health, gender, and diet of the subject, the time of administration, the route of administration, the rate of excretion, and any drug combination.

It will also be appreciated that the effective dosage of a compound of the invention or a pharmaceutically acceptable salt, prodrug, metabolite, or derivative thereof used for treatment may increase or decrease over the course of a particular treatment. Changes in dosage may result and become apparent from the results of diagnostic assays.

In some embodiments, the Cbl-b inhibitor is administered to the subject at a dose of between about 0.001 g/kg and about 1000 mg/kg, including but not limited to about 0.001 g/kg, 0.01 g/kg, 0.05 g/kg, 0.1 g/kg, 0.5 g/kg, 1 g/kg, 10 g/kg, 25 g/kg, 50 g/kg, 100 g/kg, 250 g/kg, 500 g/kg, 1 mg/kg, 5 mg/kg, 10 mg/kg, 25 mg/kg, 50 mg/kg, 100 mg/kg, and 200 mg/kg.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

The term “administration” or “administering” includes routes of introducing the compound(s) to a subject to perform their intended function. Examples of routes of administration which can be used include injection (subcutaneous, intravenous, parenterally, intraperitoneally, intrathecal), topical, oral, inhalation, rectal and transdermal.

The term “effective amount” includes an amount effective, at dosages and for periods of time necessary, to achieve the desired result. An effective amount of compound may vary according to factors such as the disease state, age, and weight of the subject, and the ability of the compound to elicit a desired response in the subject. Dosage regimens may be adjusted to provide the optimum therapeutic response.

The phrases “systemic administration,” “administered systemically”, “peripheral administration” and “administered peripherally” as used herein mean the administration of a compound(s), drug or other material, such that it enters the patient's system and, thus, is subject to metabolism and other like processes.

The phrase “therapeutically effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “subject” refers to animals such as mammals, including, but not limited to, primates (e.g., humans), cows, sheep, goats, horses, dogs, cats, rabbits, rats, mice and the like. In certain embodiments, the subject is a human.

### Combination Therapies

Depending upon the particular condition, or disease, to be treated, additional therapeutic agents, which are normally administered to treat that condition, may be administered in combination with compounds and compositions of this invention. As used herein, additional therapeutic agents that are normally administered to treat a particular disease, or condition, are known as “appropriate for the disease, or condition, being treated.”

In certain embodiments, a provided combination, or composition thereof, is administered in combination with another therapeutic agent. Examples of agents the combinations of this invention may also be combined with include, without limitation: treatments for Alzheimer's Disease such as Aricept® and Excelon®; treatments for HIV such as ritonavir; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif©), Copaxone®, and mitoxantrone; treatments for asthma such as albuterol and Singulair®; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; agents that prolong or improve pharmacokinetics such as cytochrome P450 inhibitors (i.e., inhibitors of metabolic breakdown) and CYP3A4 inhibitors (e.g., ketokenozole and ritonavir), and agents for treating immunodeficiency disorders such as gamma globulin.

In certain embodiments, combination therapies of the present invention, or a pharmaceutically acceptable composition thereof, are administered in combination with a monoclonal antibody or an siRNA therapeutic.

Those additional agents may be administered separately from a provided combination therapy, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a combination of the present invention may be administered with another therapeutic agent simultaneously or sequentially in separate unit dosage forms or together in a single unit dosage form.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

In one embodiment, the present invention provides a composition comprising a compound of formula I and one or more additional therapeutic agents. The therapeutic agent may be administered together with a compound of formula I, or may be administered prior to or following administration of a compound of formula I. Suitable therapeutic agents are described in further detail below. In certain embodiments, a compound of formula I may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, a compound of formula I may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

In some embodiments, the present invention provides a method of treating a metabolic disorder, disease, or condition described herein, comprising administering a compound of the invention in conjunction with one or more pharmaceutical agents. Suitable pharmaceutical agents that may be used in combination with the compounds of the present invention include anti-diabetic agents, anti-obesity agents (including appetite suppressants), anti-hyperglycemic agents, lipid lowering agents, and anti-hypertensive agents.

Suitable anti-diabetic agents that can be used in conjunction with a provided compound or composition thereof include but are not limited to other acetyl-CoA carboxylase (ACC) inhibitors, DGAT-1 inhibitors, AZD7687, LCQ908, DGAT-2 inhibitors, monoacylglycerol O-acyltransferase inhibitors, PDE-10 inhibitors, AMPK activators, sulfonylureas (e.g. acetohexamide, chlorpropamide, diabinese, glibenclamide, glipizide, glyburide, blimipiride, gliclazide, glipentide, gliquidone, glisolamide, tolazamide, tolbutamide), meglitinides, alpha-amylase inhibitors (e.g. tendamistat, treastatin, AL-3688), alpha-glucoside hydrolase inhibitors (e.g. acarbose), alpha-glucosidase inhibitors (e.g. adiposine, camiglibose, emiglitate, miglitol, voglibose, pradimicin-Q, sarbostatin), PPAR-gamma agonists (e.g. balaglitazone, ciglitazone, darglitazone, englitazone, isaglitazone, pioglitazone, rosiglitazone, troglitazone), PPAR-alpha/gamma agonists (e.g. CLX-0940, GW-1536, GW-1929, GW-2433, KRP-297, L-796449, LR-90, MK-0767, SB-219994), biguanides (e.g. metformin, buformin), GLP-1 modulators (exendin-3, exendin-4), liraglutide, albiglutide, exenatide (Byetta), taspoglutide, lixisenatide, dulaglutide, semaglutide, N,N-9924, TTP-054, PTP-1B inhibitors (trodusquemine, hyrtiosal extract), SIRT-1 inhibitors (e.g. resveratrol, GSK2245840, GSK184072), DPP-IV inhibitors (e.g. sitagliptin, vildagliptin, alogliptin, dutogliptin, linagliptin, saxagliptin), insulin secretagogues, fatty acid oxidation inhibitors, A2 antagonists, JNK inhibitors, glucokinase activators (e.g. TTP-399, TTP-355, TTP-547, AZD1656, ARRY403, MK-0599, TAK-329, AZD5658, GKM-001), insulin, insulin mimetics, glycogen phosphorylase inhibitors (e.g. GSK1362885), VPAC2 receptor agonists, SGLT2 inhibitors (dapagliflozin, canagliflozin, BI-10733, tofogliflozin, ASP-1941, THR1474, TS-071, ISIS388626, LX4211), glucagon receptor modulators, GPR119 modulators (e.g. MBX-2982, GSK1292263, APD597, PSN821), FGF21 derivatives, TGR5 (GPBAR1) receptor agonists (e.g. INT777), GPR40 agonists (e.g. TAK-875), GPR120 agonists, nicotinic acid receptor (HM74A) activators, SGLT1 inhibitors (e.g. GSK1614235), carnitine palmitoyl transferase enzyme inhibitors, fructose 1,6-diphosphatase inhibitors, aldose reductase inhibitors, mineralocorticoid receptor inhibitors, TORC2 inhibitors, CCR2 inhibitors, CCR5 inhibitors, PKC (e.g. PKC-alpha, PKC-beta, PKC-gamma) inhibitors, fatty acid synthetase inhibitors, seine palmitoyl transferase inhibitors, GPR81 modulators, GPR39 modulators, GPR43 modulators, GPR41 modulators, GPR105 modulators, Kv1.3 inhibitors, retinol binding protein 4 inhibitors, glucocorticoid receptor modulators, somatostatin receptor (e.g. SSTR1, SSTR2, SSTR3, SSTR5) inhibitors, PDHK2 inhibitors, PDHK4 inhibitors, MAP4K4 inhibitors, IL1-beta modulators, and RXR-alpha modulators.

Suitable lipid lowering agents that can be used in conjunction with a provided compound or composition thereof include but are not limited to, bile acid sequestrants, HMG-CoA reductase inhibitors, HMG-CoA synthase inhibitors, cholesterol absorption inhibitors, acyl coenzyme A-cholesterol acyl transferase (ACAT) inhibitors, CETP inhibitors, squalene synthetase inhibitors, PPAR-alpha agonists, FXR receptor modulators, LXR receptor modulators, lipoprotein synthesis inhibitors, renin-angiotensin system inhibitors, PPAR-delta partial agonists, bile acid reabsorption inhibitors, PPAR-gamma agonists, triglyceride synthesis inhibitors, microsomal triglyceride transport inhibitors, transcription modulators, squalene epoxidase inhibitors, low density lipoprotein receptor inducers, platelet aggregation inhibitors, 5-LO or FLAP inhibitors, niacin, and niacin-bound chromium.

Suitable anti-obesity agents include but are not limited to, 11-beta-hydroxysteroid dehydrogenase 1 inhibitors, stearoyl-CoA desaturase (SCD-1) inhibitors, MCR-4 agonists, CCK-A agonists, monoamine reuptake inhibitors (e.g. sibutramine), sympathomimetic agents, beta-3-adrenergic receptor agonists, dopamine receptor agonists (e.g. bromocriptine), melanocyte-stimulating hormone and analogs thereof, 5-HT2c agonists (e.g. lorcaserin/Belviq), melanin concentrating hormone antagonists, leptin, leptin analogs, leptin agonists, galanin antagonists, lipase inhibitors (e.g. tetrahydrolipstatin/Orlistat), anorectic agents (e.g. bombesin agonists), NPY antagonists (e.g. velneperit), PYY3-36 (and analogs thereof), BRS3 modulators, opioid receptor mixed antagonists, thyromimetic agents, dehydroepiandrosterone, glucocorticoid agonists or antagonists, orexin antagonists, GLP-1 agonists, ciliary neurotrophic factors (e.g. Axokine), human agouti-related protein (AGRP) inhibitors, H3 antagonists or inverse agonists, neuromedin U agonists, MTP/ApoB inhibitors (e.g. gut-selective MTP inhibitors such as dirlotapide, JTT130, Usistapide, SLX4090), MetAp2 inhibitors (e.g. ZGN-433), agents with mixed modulatory activity at two or more of glucagon, GIP, and GLP1 receptors (e.g. MAR-701, ZP2929), norepinephrine reuptake inhibitors, opioid antagonists (e.g. naltrexone), CB1 receptor antagonists or inverse agonists, ghrelin agonists or antagonists, oxyntomodulin and analogs thereof, monoamine uptake inhibitors (e.g. tesofensine), and combination agents (e.g. buproprion plus zonisamide (Empatic), pramlintide plus metreleptin, buproprion plus naltrexone (Contrave), phentermine plus topiramate (Qsymia).

In some embodiments, the anti-obesity agents used in combination with a provided compound or composition thereof are selected from gut-selective MTP inhibitors (e.g. dirlotapide, mitratapide, implitapide, R56918), CCK-A agonists, 5-HT2C agonists (e.g. lorcaserin/Belviq), MCR4 agonists, lipase inhibitors (e.g. Cetilistat), PYY3-36 (including analogs and PEGylated analogs thereof), opioid antagonists (e.g. naltrexone), oleoyl estrone, obinepitide, pramlintide, tesofensine, leptin, bromocriptine, orlistat, AOD-9604, and sibutramine.

In another embodiment, the present invention provides a method of treating an inflammatory disease, disorder or condition by administering to a patient in need thereof a compound of formula I and one or more additional therapeutic agents. Such additional therapeutic agents may be small molecules or recombinant biologic agents and include, for example, acetaminophen, non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol, febuxostat (Uloric®), sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), canakinumab (Ilaris®), anti-Jak inhibitors such as tofacitinib, antibodies such as rituximab (Rituxan®), “anti-T-cell” agents such as abatacept (Orencia®), “anti-IL-6” agents such as tocilizumab (Actemra®), diclofenac, cortisone, hyaluronic acid (Synvisc® or Hyalgan®), monoclonal antibodies such as tanezumab, anticoagulants such as heparin (Calcinparine® or Liquaemin®) and warfarin (Coumadin®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot®, anticholinergics or antispasmodics such as dicyclomine (Bentyl®), Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as beclomethasone dipropionate (Beclovent®, Qvar®, and Vanceril®), triamcinolone acetonide (Azmacort®), mometasone (Asthmanex®), budesonide (Pulmocort®), and flunisolide (Aerobid®), Afviar®, Symbicort®, Dulera®, cromolyn sodium (Intal®), methylxanthines such as theophylline (Theo-Dur®, Theolair®, Slo-bid®, Uniphyl®, Theo-24®) and aminophylline, IgE antibodies such as omalizumab (Xolair®), nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), doxorubicin (Hydrodaunorubicin®), vincristine (Oncovin®), bortezomib (Velcade®), and dexamethasone (Decadron®) in combination with lenalidomide (Revlimid®), or any combination(s) thereof.

In another embodiment, the present invention provides a method of treating a hematological malignancy comprising administering to a patient in need thereof a compound of formula I and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a PI3K inhibitor, a SYK inhibitor, and combinations thereof.

In another embodiment, the present invention provides a method of treating a solid tumor comprising administering to a patient in need thereof a compound of formula I and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a PI3K inhibitor, a SYK inhibitor, and combinations thereof.

In another embodiment, the present invention provides a method of treating a hematological malignancy comprising administering to a patient in need thereof a compound of formula I and a Hedgehog (Hh) signaling pathway inhibitor. In some embodiments, the hematological malignancy is DLBCL (Ramirez et al “Defining causative factors contributing in the activation of hedgehog signaling in diffuse large B-cell lymphoma” Leuk. Res. (2012), published online July 17, and incorporated herein by reference in its entirety).

In another embodiment, the present invention provides a method of treating diffuse large B-cell lymphoma (DLBCL) comprising administering to a patient in need thereof a compound of formula I and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, and combinations thereof.

In another embodiment, the present invention provides a method of treating multiple myeloma comprising administering to a patient in need thereof a compound of formula I and one or more additional therapeutic agents selected from bortezomib (Velcade®), and dexamethasone (Decadron®), a hedgehog signaling inhibitor, a BTK inhibitor, a JAK/pan-JAK inhibitor, a TYK2 inhibitor, a PI3K inhibitor, a SYK inhibitor in combination with lenalidomide (Revlimid®).

In another embodiment, the present invention provides a method of treating or lessening the severity of a disease comprising administering to a patient in need thereof a compound of formula I and a BTK inhibitor, wherein the disease is selected from inflammatory bowel disease, arthritis, cutaneous lupus erythematosus, systemic lupus erythematosus (SLE), vasculitis, idiopathic thrombocytopenic purpura (ITP), rheumatoid arthritis, psoriatic arthritis, osteoarthritis, Still's disease, juvenile arthritis, diabetes, myasthenia gravis, Hashimoto's thyroiditis, Ord's thyroiditis, Graves' disease, autoimmune thyroiditis, Sjogren's syndrome, multiple sclerosis, systemic sclerosis, Lyme neuroborreliosis, Guillain-Barre syndrome, acute disseminated encephalomyelitis, Addison's disease, opsoclonus-myoclonus syndrome, ankylosing spondylosis, antiphospholipid antibody syndrome, aplastic anemia, autoimmune hepatitis, autoimmune gastritis, pernicious anemia, celiac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behcet's disease, chronic fatigue, dysautonomia, membranous glomerulonephropathy, endometriosis, interstitial cystitis, pemphigus vulgaris, bullous pemphigoid, neuromyotonia, scleroderma, vulvodynia, a hyperproliferative disease, rejection of transplanted organs or tissues, Acquired Immunodeficiency Syndrome (AIDS, also known as HIV), type 1 diabetes, graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis, asthma, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis, B-cell proliferative disorder, e.g., diffuse large B cell lymphoma, follicular lymphoma, chronic lymphocytic lymphoma, chronic lymphocytic leukemia, acute lymphocytic leukemia, B-cell prolymphocytic leukemia, lymphoplasmacytic lymphoma/Waldenstrom macroglobulinemia, splenic marginal zone lymphoma, multiple myeloma (also known as plasma cell myeloma), non-Hodgkin's lymphoma, Hodgkin's lymphoma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, Burkitt lymphoma/leukemia, or lymphomatoid granulomatosis, breast cancer, prostate cancer, or cancer of the mast cells (e.g., mastocytoma, mast cell leukemia, mast cell sarcoma, systemic mastocytosis), bone cancer, colorectal cancer, pancreatic cancer, diseases of the bone and joints including, without limitation, rheumatoid arthritis, seronegative spondyloarthropathies (including ankylosing spondylitis, psoriatic arthritis and Reiter's disease), Behcet's disease, Sjogren's syndrome, systemic sclerosis, osteoporosis, bone cancer, bone metastasis, a thromboembolic disorder, (e.g., myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, deep venous thrombosis), inflammatory pelvic disease, urethritis, skin sunburn, sinusitis, pneumonitis, encephalitis, meningitis, myocarditis, nephritis, osteomyelitis, myositis, hepatitis, gastritis, enteritis, dermatitis, gingivitis, appendicitis, pancreatitis, cholocystitus, agammaglobulinemia, psoriasis, allergy, Crohn's disease, irritable bowel syndrome, ulcerative colitis, Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anemia, glomerulonephritis, dermatomyositis, multiple sclerosis, scleroderma, vasculitis, autoimmune hemolytic and thrombocytopenic states, Goodpasture's syndrome, atherosclerosis, Addison's disease, Parkinson's disease, Alzheimer's disease, diabetes, septic shock, cutaneous lupus erythematosus, systemic lupus erythematosus (SLE), rheumatoid arthritis, psoriatic arthritis, juvenile arthritis, osteoarthritis, chronic idiopathic thrombocytopenic purpura, Waldenstrom macroglobulinemia, myasthenia gravis, Hashimoto's thyroiditis, atopic dermatitis, degenerative joint disease, vitiligo, autoimmune hypopituitarism, Guillain-Barre syndrome, Behcet's disease, scleraderma, mycosis fungoides, acute inflammatory responses (such as acute respiratory distress syndrome and ischemia/reperfusion injury), and Graves' disease.

In another embodiment, the present invention provides a method of treating or lessening the severity of a disease comprising administering to a patient in need thereof a compound of formula I and a PI3K inhibitor, wherein the disease is selected from a cancer, a neurodegenative disorder, an angiogenic disorder, a viral disease, an autoimmune disease, an inflammatory disorder, a hormone-related disease, conditions associated with organ transplantation, immunodeficiency disorders, a destructive bone disorder, a proliferative disorder, an infectious disease, a condition associated with cell death, thrombin-induced platelet aggregation, chronic myelogenous leukemia (CML), chronic lymphocytic leukemia (CLL), liver disease, pathologic immune conditions involving T cell activation, a cardiovascular disorder, and a CNS disorder.

In another embodiment, the present invention provides a method of treating or lessening the severity of a disease comprising administering to a patient in need thereof a compound of formula I and a PI3K inhibitor, wherein the disease is selected from benign or malignant tumor, carcinoma or solid tumor of the brain, kidney (e.g., renal cell carcinoma (RCC)), liver, adrenal gland, bladder, breast, stomach, gastric tumors, ovaries, colon, rectum, prostate, pancreas, lung, vagina, endometrium, cervix, testis, genitourinary tract, esophagus, larynx, skin, bone or thyroid, sarcoma, glioblastomas, neuroblastomas, multiple myeloma or gastrointestinal cancer, especially colon carcinoma or colorectal adenoma or a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, (including, for example, non-Hodgkin's Lymphoma (NHL) and Hodgkin's lymphoma (also termed Hodgkin's or Hodgkin's disease)), a mammary carcinoma, follicular carcinoma, undifferentiated carcinoma, papillary carcinoma, seminoma, melanoma, or a leukemia, diseases include Cowden syndrome, Lhermitte-Dudos disease and Bannayan-Zonana syndrome, or diseases in which the PI3K/PKB pathway is aberrantly activated, asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection, acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy, bronchitis of whatever type or genesis including, but not limited to, acute, arachidic, catarrhal, croupus, chronic or phthinoid bronchitis, pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis, Loffler's syndrome, eosinophilic, pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction, psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, pemphisus, epidermolysis bullosa acquisita, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), cutaneous lupus erythematosus, systemic lupus erythematosus, rheumatoid arthritis, polychondritis, sclerodoma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine opthalmopathy, Grave's disease, sarcoidosis, alveolitis, chronic hypersensitivity pneumonitis, multiple sclerosis, primary biliary cirrhosis, uveitis (anterior and posterior), keratoconjunctivitis sicca and vernal keratoconjunctivitis, interstitial lung fibrosis, psoriatic arthritis and glomerulonephritis (with and without nephrotic syndrome, e.g. including idiopathic nephrotic syndrome or minal change nephropathy, restenosis, cardiomegaly, atherosclerosis, myocardial infarction, ischemic stroke and congestive heart failure, Alzheimer's disease, Parkinson's disease, amyotrophic lateral sclerosis, Huntington's disease, and cerebral ischemia, and neurodegenerative disease caused by traumatic injury, glutamate neurotoxicity and hypoxia.

In some embodiments the present invention provides a method of treating or lessening the severity of a disease comprising administering to a patient in need thereof a compound of formula I and a Bcl-2 inhibitor, wherein the disease is an inflammatory disorder, an autoimmune disorder, a proliferative disorder, an endocrine disorder, a neurological disorder, or a disorder associated with transplantation. In some embodiments, the disorder is a proliferative disorder, lupus, or lupus nephritis. In some embodiments, the proliferative disorder is chronic lymphocytic leukemia, diffuse large B-cell lymphoma, Hodgkin's disease, small-cell lung cancer, non-small-cell lung cancer, myelodysplastic syndrome, lymphoma, a hematological neoplasm, or solid tumor.

In some embodiments, the disease is an autoimmune disorder, an inflammatory disorder, a proliferative disorder, an endocrine disorder, a neurological disorder, or a disorder associated with transplantation. In some embodiments the JH2 binding compound is a compound of formula I. Other suitable JH2 domain binding compounds include those described in WO2014074660A1, WO2014074661A1, WO2015089143A1, the entirety of each of which is incorporated herein by reference. Suitable JH1 domain binding compounds include those described in WO2015131080A1, the entirety of which is incorporated herein by reference.

A compound of the current invention may also be used to advantage in combination with other therapeutic compounds. In some embodiments, the other therapeutic compounds are antiproliferative compounds. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (Temodal©); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZD6244 from AstraZeneca, PD181461 from Pfizer and leucovorin. The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name Aromasin™ Formestane is marketed under the trade name Lentaron™. Fadrozole is marketed under the trade name Afema™. Anastrozole is marketed under the trade name Arimidex™. Letrozole is marketed under the trade names Femara™ or Femar™. Aminoglutethimide is marketed under the trade name Orimeten™. A combination of the invention comprising a chemotherapeutic agent which is an aromatase inhibitor is particularly useful for the treatment of hormone receptor positive tumors, such as breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name Nolvadex™ Raloxifene hydrochloride is marketed under the trade name Evista™. Fulvestrant can be administered under the trade name Faslodex™. A combination of the invention comprising a chemotherapeutic agent which is an antiestrogen is particularly useful for the treatment of estrogen receptor positive tumors, such as breast tumors.

The term “anti-androgen” as used herein relates to any substance which is capable of inhibiting the biological effects of androgenic hormones and includes, but is not limited to, bicalutamide (Casodex™) The term “gonadorelin agonist” as used herein includes, but is not limited to abarelix, goserelin and goserelin acetate. Goserelin can be administered under the trade name Zoladex™.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148. Irinotecan can be administered, e.g. in the form as it is marketed, e.g. under the trademark Camptosar™. Topotecan is marketed under the trade name Hycamptin™.

The term “topoisomerase II inhibitor” as used herein includes, but is not limited to the anthracyclines such as doxorubicin (including liposomal formulation, such as Caelyx™), daunorubicin, epirubicin, idarubicin and nemorubicin, the anthraquinones mitoxantrone and losoxantrone, and the podophillotoxines etoposide and teniposide. Etoposide is marketed under the trade name Etopophos™ Teniposide is marketed under the trade name VM 26-Bristol Doxorubicin is marketed under the trade name Acriblastin™ or Adriamycin™. Epirubicin is marketed under the trade name Farmorubicin™. Idarubicin is marketed. under the trade name Zavedos™. Mitoxantrone is marketed under the trade name Novantron.

The term “microtubule active agent” relates to microtubule stabilizing, microtubule destabilizing compounds and microtublin polymerization inhibitors including, but not limited to taxanes, such as paclitaxel and docetaxel; vinca alkaloids, such as vinblastine or vinblastine sulfate, vincristine or vincristine sulfate, and vinorelbine; discodermolides; cochicine and epothilones and derivatives thereof. Paclitaxel is marketed under the trade name Taxol™. Docetaxel is marketed under the trade name Taxotere™. Vinblastine sulfate is marketed under the trade name Vinblastin R.P™. Vincristine sulfate is marketed under the trade name Farmistin™.

The term “alkylating agent” as used herein includes, but is not limited to, cyclophosphamide, ifosfamide, melphalan or nitrosourea (BCNU or Gliadel). Cyclophosphamide is marketed under the trade name Cyclostin™. Ifosfamide is marketed under the trade name Holoxan™.

The term “histone deacetylase inhibitors” or “HDAC inhibitors” relates to compounds which inhibit the histone deacetylase and which possess antiproliferative activity. This includes, but is not limited to, suberoylanilide hydroxamic acid (SAHA).

The term “antineoplastic antimetabolite” includes, but is not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name Xeloda™. Gemcitabine is marketed under the trade name Gemzar™.

The term “platin compound” as used herein includes, but is not limited to, carboplatin, cis-platin, cisplatinum and oxaliplatin. Carboplatin can be administered, e.g., in the form as it is marketed, e.g. under the trademark Carboplat™. Oxaliplatin can be administered, e.g., in the form as it is marketed, e.g. under the trademark Eloxatin™.

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the AxI receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Abl family, their gene-fusion products (e.g. BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt, Ras/MAPK, PI3K, SYK, BTK and TEC family, and/or members of the cyclin-dependent kinase family (CDK) including staurosporine derivatives, such as midostaurin; examples of further compounds include UCN-01, safingol, BAY 43-9006, Bryostatin 1, Perifosine; llmofosine; RO 318220 and RO 320432; GO 6976; lsis 3521; LY333531/LY379196; isochinoline compounds; FTIs; PD184352 or QAN697 (a P13K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (Gleevec™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) compounds targeting, decreasing or inhibiting the activity of the epidermal growth factor family of receptor tyrosine kinases (EGFR₁ ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774, ZD 1839, ZM 105180; trastuzumab (Herceptin™), cetuximab (Erbitux™), Iressa, Tarceva, OSI-774, C₁₋₁₀33, EKB-569, GW-2016, ELI, E2.4, E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAK1/JAK2/JAK3/TYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (PI3K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib; and; and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

The term “PI3K inhibitor” as used herein includes, but is not limited to compounds having inhibitory activity against one or more enzymes in the phosphatidylinositol-3-kinase family, including, but not limited to PI3Kα, PI3Kγ, PI3Kδ, PI3Kβ, PI3K-C2α, PI3K-C2β, PI3K-C2γ, Vps34, p110-α, p110-β, p110-γ, p110-δ, p85-α, p85-β, p55-γ, p150, p101, and p87. Examples of PI3K inhibitors useful in this invention include but are not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib.

The term “Bcl-2 inhibitor” as used herein includes, but is not limited to compounds having inhibitory activity against B-cell lymphoma 2 protein (Bcl-2), including but not limited to ABT-199, ABT-731, ABT-737, apogossypol, Ascenta's pan-Bcl-2 inhibitors, curcumin (and analogs thereof), dual Bcl-2/Bcl-xL inhibitors (Infinity Pharmaceuticals/Novartis Pharmaceuticals), Genasense (G3139), HA14-1 (and analogs thereof; see WO2008118802), navitoclax (and analogs thereof, see U.S. Pat. No. 7,390,799), NH—1 (Shenayng Pharmaceutical University), obatoclax (and analogs thereof, see WO2004106328), S-001 (Gloria Pharmaceuticals), TW series compounds (Univ. of Michigan), and venetoclax. In some embodiments the Bcl-2 inhibitor is a small molecule therapeutic. In some embodiments the Bcl-2 inhibitor is a peptidomimetic.

Further examples of BTK inhibitory compounds, and conditions treatable by such compounds in combination with compounds of this invention can be found in WO2008039218 and WO2011090760, the entirety of which are incorporated herein by reference.

Further examples of SYK inhibitory compounds, and conditions treatable by such compounds in combination with compounds of this invention can be found in WO2003063794, WO2005007623, and WO2006078846, the entirety of which are incorporated herein by reference.

Further examples of PI3K inhibitory compounds, and conditions treatable by such compounds in combination with compounds of this invention can be found in WO2004019973, WO2004089925, WO2007016176, U.S. Pat. No. 8,138,347, WO2002088112, WO2007084786, WO2007129161, WO2006122806, WO2005113554, and WO2007044729 the entirety of which are incorporated herein by reference.

Further examples of JAK inhibitory compounds, and conditions treatable by such compounds in combination with compounds of this invention can be found in WO2009114512, WO2008109943, WO2007053452, WO2000142246, and WO2007070514, the entirety of which are incorporated herein by reference.

Further anti-angiogenic compounds include compounds having another mechanism for their activity, e.g. unrelated to protein or lipid kinase inhibition e.g. thalidomide (Thalomid™) and TNP-470.

Examples of proteasome inhibitors useful for use in combination with compounds of the invention include, but are not limited to bortezomib, disulfiram, epigallocatechin-3-gallate (EGCG), salinosporamide A, carfilzomib, ONX-0912, CEP-18770, and MLN9708.

Compounds which target, decrease or inhibit the activity of a protein or lipid phosphatase are e.g. inhibitors of phosphatase 1, phosphatase 2A, or CDC25, such as okadaic acid or a derivative thereof.

Compounds which induce cell differentiation processes include, but are not limited to, retinoic acid, α- γ- or δ-tocopherol or α- γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (Celebrex™), rofecoxib (Vioxx™), etoricoxib, valdecoxib or a 5-alkyl-2-arylaminophenylacetic acid, such as 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. Etridonic acid is marketed under the trade name Didronel™. Clodronic acid is marketed under the trade name Bonefos™. Tiludronic acid is marketed under the trade name Skelid™. Pamidronic acid is marketed under the trade name Aredia™. Alendronic acid is marketed under the trade name Fosamax™. Ibandronic acid is marketed under the trade name Bondranat™. Risedronic acid is marketed under the trade name Actonel™. Zoledronic acid is marketed under the trade name Zometa™. The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “heparanase inhibitor” as used herein refers to compounds which target, decrease or inhibit heparin sulfate degradation. The term includes, but is not limited to, PI-88. The term “biological response modifier” as used herein refers to a lymphokine or interferons.

The term “inhibitor of Ras oncogenic isoforms”, such as H-Ras, K-Ras, or N-Ras, as used herein refers to compounds which target, decrease or inhibit the oncogenic activity of Ras; for example, a “farnesyl transferase inhibitor” such as L-744832, DK8G557 or R115777 (Zarnestra™). The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “proteasome inhibitor” as used herein refers to compounds which target, decrease or inhibit the activity of the proteasome. Compounds which target, decrease or inhibit the activity of the proteasome include, but are not limited to, Bortezomib (Velcade™) and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors, which are compounds targeting, decreasing or inhibiting the activity of FMS-like tyrosine kinase receptors (Flt-3R); interferon, 1-β-D-arabinofuransylcytosine (ara-c) and bisulfan; ALK inhibitors, which are compounds which target, decrease or inhibit anaplastic lymphoma kinase, and Bcl-2 inhibitors.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino,17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (Herceptin™), Trastuzumab-DM1, erbitux, bevacizumab (Avastin™), rituximab (Rituxan®), PRO64553 (anti-CD40) and 2C4 Antibody. By antibodies is meant intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of the current invention can be used in combination with standard leukemia therapies, especially in combination with therapies used for the treatment of AML. In particular, compounds of the current invention can be administered in combination with, for example, farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412. In some embodiments, the present invention provides a method of treating AML associated with an ITD and/or D835Y mutation, comprising administering a compound of the present invention together with a one or more FLT3 inhibitors. In some embodiments, the FLT3 inhibitors are selected from quizartinib (AC220), a staurosporine derivative (e.g. midostaurin or lestaurtinib), sorafenib, tandutinib, LY-2401401, LS-104, EB-10, famitinib, NOV-110302, NMS-P948, AST-487, G-749, SB-1317, S-209, SC-110219, AKN-028, fedratinib, tozasertib, and sunitinib. In some embodiments, the FLT3 inhibitors are selected from quizartinib, midostaurin, lestaurtinib, sorafenib, and sunitinib.

Other anti-leukemic compounds include, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065 including, but not limited to, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt. Somatostatin receptor antagonists as used herein refer to compounds which target, treat or inhibit the somatostatin receptor such as octreotide, and SOM230. Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol. 1, pp. 248-275 (1993).

Also included are EDG binders and ribonucleotide reductase inhibitors. The term “EDG binders” as used herein refers to a class of immunosuppressants that modulates lymphocyte recirculation, such as FTY720. The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives.

Also included are in particular those compounds, proteins or monoclonal antibodies of VEGF such as 1-(4-chloroanilino)-4-(4-pyridylmethyl)phthalazine or a pharmaceutically acceptable salt thereof, 1-(4-chloroanilino)-4-(4-pyridylmethyl)phthalazine succinate; Angiostatin™; Endostatin™; anthranilic acid amides; ZD4190; ZD6474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgGI antibody, Angiozyme (RPI 4610) and Bevacizumab (Avastin™)

Photodynamic therapy as used herein refers to therapy which uses certain chemicals known as photosensitizing compounds to treat or prevent cancers. Examples of photodynamic therapy include treatment with compounds, such as Visudyne™ and porfimer sodium.

Angiostatic steroids as used herein refers to compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as fluocinolone and dexamethasone.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The compounds of the invention are also useful as co-therapeutic compounds for use in combination with other drug substances such as anti-inflammatory, bronchodilatory or antihistamine drug substances, particularly in the treatment of obstructive or inflammatory airways diseases such as those mentioned hereinbefore, for example as potentiators of therapeutic activity of such drugs or as a means of reducing required dosaging or potential side effects of such drugs. A compound of the invention may be mixed with the other drug substance in a fixed pharmaceutical composition or it may be administered separately, before, simultaneously with or after the other drug substance. Accordingly the invention includes a combination of a compound of the invention as hereinbefore described with an anti-inflammatory, bronchodilatory, antihistamine or anti-tussive drug substance, said compound of the invention and said drug substance being in the same or different pharmaceutical composition.

Suitable anti-inflammatory drugs include steroids, in particular glucocorticosteroids such as budesonide, beclamethasone dipropionate, fluticasone propionate, ciclesonide or mometasone furoate; non-steroidal glucocorticoid receptor agonists; LTB4 antagonists such LY293111, CGS025019C, CP-195543, SC-53228, BIIL 284, ONO 4057, SB 209247; LTD4 antagonists such as montelukast and zafirlukast; PDE4 inhibitors such cilomilast (Ariflo® GlaxoSmithKline), Roflumilast (Byk Gulden),V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SeICID™ CC-10004 (Celgene), VM554/UM565 (Vernalis), T-440 (Tanabe), KW-4490 (Kyowa Hakko Kogyo); A2a agonists; A2b antagonists; and beta-2 adrenoceptor agonists such as albuterol (salbutamol), metaproterenol, terbutaline, salmeterol fenoterol, procaterol, and especially, formoterol and pharmaceutically acceptable salts thereof. Suitable bronchodilatory drugs include anticholinergic or antimuscarinic compounds, in particular ipratropium bromide, oxitropium bromide, tiotropium salts and CHF 4226 (Chiesi), and glycopyrrolate.

Suitable antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine.

Other useful combinations of compounds of the invention with anti-inflammatory drugs are those with antagonists of chemokine receptors, e.g. CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists such as Schering-Plough antagonists SC-351125, SCH-55700 and SCH-D, and Takeda antagonists such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-aminium chloride (TAK-770).

The structure of the active compounds identified by code numbers, generic or trade names may be taken from the actual edition of the standard compendium “The Merck Index” or from databases, e.g. Patents International (e.g. IMS World Publications).

### Exemplary Immuno-Oncology Agents

In some embodiments, one or more other therapeutic agent is an immuno-oncology agent. As used herein, the term “an immuno-oncology agent” refers to an agent which is effective to enhance, stimulate, and/or up-regulate immune responses in a subject. In some embodiments, the administration of an immuno-oncology agent with a compound of the invention has a synergic effect in treating a cancer.

An immuno-oncology agent can be, for example, a small molecule drug, an antibody, or a biologic or small molecule. Examples of biologic immuno-oncology agents include, but are not limited to, cancer vaccines, antibodies, and cytokines. In some embodiments, an antibody is a monoclonal antibody. In some embodiments, a monoclonal antibody is humanized or human.

In some embodiments, an immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses.

Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LTβR, LIGHT, DcR3, HVEM, VEGI/TL1A, TRAMP/DR3, EDAR, EDA1, XEDAR, EDA2, TNFR1, Lymphotoxin α/TNFβ, TNFR2, TNFα, LTβR, Lymphotoxin α1β2, FAS, FASL, RELT, DR6, TROY, NGFR.

In some embodiments, an immuno-oncology agent is a cytokine that inhibits T cell activation (e.g., IL-6, IL-10, TGF-β, VEGF, and other immunosuppressive cytokines) or a cytokine that stimulates T cell activation, for stimulating an immune response.

In some embodiments, a combination of a compound of the invention and an immuno-oncology agent can stimulate T cell responses. In some embodiments, an immuno-oncology agent is: (i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIR1, TIM-1, and TIM-4; or (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD28H.

In some embodiments, an immuno-oncology agent is an antagonist of inhibitory receptors on NK cells or an agonists of activating receptors on NK cells. In some embodiments, an immuno-oncology agent is an antagonist of KIR, such as lirilumab.

In some embodiments, an immuno-oncology agent is an agent that inhibits or depletes macrophages or monocytes, including but not limited to CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13169264; WO14/036357).

In some embodiments, an immuno-oncology agent is selected from agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell energy or exhaustion) and agents that trigger innate immune activation and/or inflammation at tumor sites.

In some embodiments, an immuno-oncology agent is a CTLA-4 antagonist. In some embodiments, a CTLA-4 antagonist is an antagonistic CTLA-4 antibody. In some embodiments, an antagonistic CTLA-4 antibody is YERVOY (ipilimumab) or tremelimumab.

In some embodiments, an immuno-oncology agent is a PD-1 antagonist. In some embodiments, a PD-1 antagonist is administered by infusion. In some embodiments, an immuno-oncology agent is an antibody or an antigen-binding portion thereof that binds specifically to a Programmed Death-1 (PD-1) receptor and inhibits PD-1 activity. In some embodiments, a PD-1 antagonist is an antagonistic PD-1 antibody. In some embodiments, an antagonistic PD-1 antibody is OPDIVO (nivolumab), KEYTRUDA (pembrolizumab), or MEDI-0680 (AMP-514; WO2012/145493). In some embodiments, an immuno-oncology agent may be pidilizumab (CT-011). In some embodiments, an immuno-oncology agent is a recombinant protein composed of the extracellular domain of PD-L2 (B7-DC) fused to the Fc portion of IgGI, called AMP-224.

In some embodiments, an immuno-oncology agent is a PD-L1 antagonist. In some embodiments, a PD-L1 antagonist is an antagonistic PD-L1 antibody. In some embodiments, a PD-L1 antibody is MPDL3280A (RG7446; WO2010/077634), durvalumab (MED14736), BMS-936559 (WO2007/005874), and MSB0010718C (WO2013/79174).

In some embodiments, an immuno-oncology agent is a LAG-3 antagonist. In some embodiments, a LAG-3 antagonist is an antagonistic LAG-3 antibody. In some embodiments, a LAG3 antibody is BMS-986016 (WO10/19570, WO14/08218), or IMP-731 or IMP-321 (WO08/132601, WO009/44273).

In some embodiments, an immuno-oncology agent is a CD137 (4-1BB) agonist. In some embodiments, a CD137 (4-1BB) agonist is an agonistic CD137 antibody. In some embodiments, a CD137 antibody is urelumab or PF-05082566 (WO12/32433).

In some embodiments, an immuno-oncology agent is a GITR agonist. In some embodiments, a GITR agonist is an agonistic GITR antibody. In some embodiments, a GITR antibody is BMS-986153, BMS-986156, TRX-518 (WO006/105021, WO009/009116), or MK-4166 (WO11/028683).

In some embodiments, an immuno-oncology agent is an indoleamine (2,3)-dioxygenase (IDO) antagonist. In some embodiments, an IDO antagonist is selected from epacadostat (INCB024360, Incyte); indoximod (NLG-8189, NewLink Genetics Corporation); capmanitib (INC280, Novartis); GDC-0919 (Genentech/Roche); PF-06840003 (Pfizer); BMS.F001287 (Bristol-Myers Squibb); Phy906/KD108 (Phytoceutica); an enzyme that breaks down kynurenine (Kynase, Ikena Oncology, formerly known as Kyn Therapeutics); and NLG-919 (WO09/73620, WO009/1156652, WO11/56652, WO12/142237).

In some embodiments, an immuno-oncology agent is an OX40 agonist. In some embodiments, an OX40 agonist is an agonistic OX40 antibody. In some embodiments, an OX40 antibody is MEDI-6383 or MEDI-6469.

In some embodiments, an immuno-oncology agent is an OX40L antagonist. In some embodiments, an OX40L antagonist is an antagonistic OX40 antibody. In some embodiments, an OX40L antagonist is RG-7888 (WO06/029879).

In some embodiments, an immuno-oncology agent is a CD40 agonist. In some embodiments, a CD40 agonist is an agonistic CD40 antibody. In some embodiments, an immuno-oncology agent is a CD40 antagonist. In some embodiments, a CD40 antagonist is an antagonistic CD40 antibody. In some embodiments, a CD40 antibody is lucatumumab or dacetuzumab.

In some embodiments, an immuno-oncology agent is a CD27 agonist. In some embodiments, a CD27 agonist is an agonistic CD27 antibody. In some embodiments, a CD27 antibody is varlilumab.

In some embodiments, an immuno-oncology agent is MGA271 (to B7H3) (WO11/109400).

In some embodiments, an immuno-oncology agent is abagovomab, adecatumumab, afutuzumab, alemtuzumab, anatumomab mafenatox, apolizumab, atezolimab, avelumab, blinatumomab, BMS-936559, catumaxomab, durvalumab, epacadostat, epratuzumab, indoximod, inotuzumab ozogamicin, intelumumab, ipilimumab, isatuximab, lambrolizumab, MED14736, MPDL3280A, nivolumab, obinutuzumab, ocaratuzumab, ofatumumab, olatatumab, pembrolizumab, pidilizumab, rituximab, ticilimumab, samalizumab, or tremelimumab.

In some embodiments, an immuno-oncology agent is an immunostimulatory agent. For example, antibodies blocking the PD-1 and PD-L1 inhibitory axis can unleash activated tumor-reactive T cells and have been shown in clinical trials to induce durable anti-tumor responses in increasing numbers of tumor histologies, including some tumor types that conventionally have not been considered immunotherapy sensitive. See, e.g., Okazaki, T. et al. (2013) Nat. Immunol. 14, 1212-1218; Zou et al. (2016) Sci. Transl. Med. 8. The anti-PD-1 antibody nivolumab (OPDIVO®, Bristol-Myers Squibb, also known as ONO-4538, MDX1106 and BMS-936558), has shown potential to improve the overall survival in patients with RCC who had experienced disease progression during or after prior anti-angiogenic therapy.

In some embodiments, the immunomodulatory therapeutic specifically induces apoptosis of tumor cells. Approved immunomodulatory therapeutics which may be used in the present invention include pomalidomide (POMALYST®, Celgene); lenalidomide (REVLIMID®, Celgene); ingenol mebutate (PICATO®, LEO Pharma).

In some embodiments, an immuno-oncology agent is a cancer vaccine. In some embodiments, the cancer vaccine is selected from sipuleucel-T (PROVENGE®, Dendreon/Valeant Pharmaceuticals), which has been approved for treatment of asymptomatic, or minimally symptomatic metastatic castrate-resistant (hormone-refractory) prostate cancer; and talimogene laherparepvec (IMLYGIC®, BioVex/Amgen, previously known as T-VEC), a genetically modified oncolytic viral therapy approved for treatment of unresectable cutaneous, subcutaneous and nodal lesions in melanoma. In some embodiments, an immuno-oncology agent is selected from an oncolytic viral therapy such as pexastimogene devacirepvec (PexaVec/JX-594, SillaJen/formerly Jennerex Biotherapeutics), a thymidine kinase-(TK-) deficient vaccinia virus engineered to express GM-CSF, for hepatocellular carcinoma (NCT02562755) and melanoma (NCT00429312); pelareorep (REOLYSIN®, Oncolytics Biotech), a variant of respiratory enteric orphan virus (reovirus) which does not replicate in cells that are not RAS-activated, in numerous cancers, including colorectal cancer (NCT01622543); prostate cancer (NCT01619813); head and neck squamous cell cancer (NCT01166542); pancreatic adenocarcinoma (NCT00998322); and non-small cell lung cancer (NSCLC) (NCT 00861627); enadenotucirev (NG-348, PsiOxus, formerly known as ColoAd1), an adenovirus engineered to express a full length CD80 and an antibody fragment specific for the T-cell receptor CD3 protein, in ovarian cancer (NCT02028117); metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676); and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260); fallopian tube cancer, ovarian cancer (NCT 02759588); or CG0070 (Cold Genesys), an adenovirus engineered to express GM-CSF, in bladder cancer (NCT02365818).

In some embodiments, an immuno-oncology agent is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TG01 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFα-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8⁺ T cell response.

In some embodiments, an immuno-oncology agent is a T-cell engineered to express a chimeric antigen receptor, or CAR. The T-cells engineered to express such chimeric antigen receptor are referred to as a CAR-T cells.

CARs have been constructed that consist of binding domains, which may be derived from natural ligands, single chain variable fragments (scFv) derived from monoclonal antibodies specific for cell-surface antigens, fused to endodomains that are the functional end of the T-cell receptor (TCR), such as the CD3-zeta signaling domain from TCRs, which is capable of generating an activation signal in T lymphocytes. Upon antigen binding, such CARs link to endogenous signaling pathways in the effector cell and generate activating signals similar to those initiated by the TCR complex.

For example, in some embodiments the CAR-T cell is one of those described in U.S. Pat. No. 8,906,682 (June et al.; hereby incorporated by reference in its entirety), which discloses CAR-T cells engineered to comprise an extracellular domain having an antigen binding domain (such as a domain that binds to CD19), fused to an intracellular signaling domain of the T cell antigen receptor complex zeta chain (such as CD3 zeta). When expressed in the T cell, the CAR is able to redirect antigen recognition based on the antigen binding specificity. In the case of CD19, the antigen is expressed on malignant B cells. Over 200 clinical trials are currently in progress employing CAR-T in a wide range of indications. [https://clinicaltrials.gov/ct2/results?term=chimeric+antigen+receptors&pg=1].

In some embodiments, an immunostimulatory agent is an activator of retinoic acid receptor-related orphan receptor γ (RORγt). RORγt is a transcription factor with key roles in the differentiation and maintenance of Type 17 effector subsets of CD4+ (Th17) and CD8+ (Tc17) T cells, as well as the differentiation of IL-17 expressing innate immune cell subpopulations such as NK cells. In some embodiments, an activator of RORγt is LYC-55716 (Lycera), which is currently being evaluated in clinical trials for the treatment of solid tumors (NCT02929862).

In some embodiments, an immunostimulatory agent is an agonist or activator of a toll-like receptor (TLR). Suitable activators of TLRs include an agonist or activator of TLR9 such as SD-101 (Dynavax). SD-101 is an immunostimulatory CpG which is being studied for B-cell, follicular and other lymphomas (NCT02254772). Agonists or activators of TLR8 which may be used in the present invention include motolimod (VTX-2337, VentiRx Pharmaceuticals) which is being studied for squamous cell cancer of the head and neck (NCT02124850) and ovarian cancer (NCT02431559).

Other immuno-oncology agents that can be used in the present invention include urelumab (BMS-663513, Bristol-Myers Squibb), an anti-CD137 monoclonal antibody; varlilumab (CDX-1127, Celldex Therapeutics), an anti-CD27 monoclonal antibody; BMS-986178 (Bristol-Myers Squibb), an anti-OX40 monoclonal antibody; lirilumab (IPH2102/BMS-986015, Innate Pharma, Bristol-Myers Squibb), an anti-KIR monoclonal antibody; monalizumab (IPH2201, Innate Pharma, AstraZeneca) an anti-NKG2A monoclonal antibody; andecaliximab (GS-5745, Gilead Sciences), an anti-MMP9 antibody; MK-4166 (Merck & Co.), an anti-GITR monoclonal antibody.

In some embodiments, an immunostimulatory agent is selected from elotuzumab, mifamurtide, an agonist or activator of a toll-like receptor, and an activator of RORγt.

In some embodiments, an immunostimulatory therapeutic is recombinant human interleukin 15 (rhIL-15). rhIL-15 has been tested in the clinic as a therapy for melanoma and renal cell carcinoma (NCT01021059 and NCT01369888) and leukemias (NCT02689453). In some embodiments, an immunostimulatory agent is recombinant human interleukin 12 (rhIL-12). In some embodiments, an IL-15 based immunotherapeutic is heterodimeric IL-15 (hetIL-15, Novartis/Admune), a fusion complex composed of a synthetic form of endogenous IL-15 complexed to the soluble IL-15 binding protein IL-15 receptor alpha chain (IL15:sIL-15RA), which has been tested in Phase 1 clinical trials for melanoma, renal cell carcinoma, non-small cell lung cancer and head and neck squamous cell carcinoma (NCT02452268). In some embodiments, a recombinant human interleukin 12 (rhIL-12) is NM-IL-12 (Neumedicines, Inc.), NCT02544724, or NCT02542124.

In some embodiments, an immuno-oncology agent is selected from those descripted in Jerry L. Adams et al., “Big opportunities for small molecules in immuno-oncology,” Cancer Therapy 2015, Vol. 14, pages 603-622, the content of which is incorporated herein by reference in its entirety. In some embodiment, an immuno-oncology agent is selected from the examples described in Table 1 of Jerry L. Adams et al. In some embodiments, an immuno-oncology agent is a small molecule targeting an immuno-oncology target selected from those listed in Table 2 of Jerry L. Adams et al. In some embodiments, an immuno-oncology agent is a small molecule agent selected from those listed in Table 2 of Jerry L. Adams et al.

In some embodiments, an immuno-oncology agent is selected from the small molecule immuno-oncology agents described in Peter L. Toogood, “Small molecule immuno-oncology therapeutic agents,” Bioorganic & Medicinal Chemistry Letters 2018, Vol. 28, pages 319-329, the content of which is incorporated herein by reference in its entirety. In some embodiments, an immuno-oncology agent is an agent targeting the pathways as described in Peter L. Toogood.

In some embodiments, an immuno-oncology agent is selected from those described in Sandra L. Ross et al., “Bispecific T cell engager (BITE®) antibody constructs can mediate bystander tumor cell killing”, PLoS ONE 12(8): e0183390, the content of which is incorporated herein by reference in its entirety. In some embodiments, an immuno-oncology agent is a bispecific T cell engager (BITE®) antibody construct. In some embodiments, a bispecific T cell engager (BITE®) antibody construct is a CD19/CD3 bispecific antibody construct. In some embodiments, a bispecific T cell engager (BITE®) antibody construct is an EGFR/CD3 bispecific antibody construct. In some embodiments, a bispecific T cell engager (BITE®) antibody construct activates T cells. In some embodiments, a bispecific T cell engager (BITE®) antibody construct activates T cells, which release cytokines inducing upregulation of intercellular adhesion molecule 1 (ICAM-1) and FAS on bystander cells. In some embodiments, a bispecific T cell engager (BITE®) antibody construct activates T cells which result in induced bystander cell lysis. In some embodiments, the bystander cells are in solid tumors. In some embodiments, the bystander cells being lysed are in proximity to the BITE®-activated T cells. In some embodiment, the bystander cells comprises tumor-associated antigen (TAA) negative cancer cells. In some embodiment, the bystander cells comprise EGFR-negative cancer cells. In some embodiments, an immuno-oncology agent is an antibody which blocks the PD-L1/PD1 axis and/or CTLA4. In some embodiments, an immuno-oncology agent is an ex vivo expanded tumor-infiltrating T cell. In some embodiments, an immuno-oncology agent is a bispecific antibody construct or chimeric antigen receptors (CARs) that directly connect T cells with tumor-associated surface antigens (TAAs).

### Exemplary Immune Checkpoint Inhibitors

In some embodiments, an immuno-oncology agent is an immune checkpoint inhibitor as described herein.

The term “checkpoint inhibitor” as used herein relates to agents useful in preventing cancer cells from avoiding the immune system of the patient. One of the major mechanisms of anti-tumor immunity subversion is known as “T-cell exhaustion,” which results from chronic exposure to antigens that has led to up-regulation of inhibitory receptors. These inhibitory receptors serve as immune checkpoints in order to prevent uncontrolled immune reactions.

PD-1 and co-inhibitory receptors such as cytotoxic T-lymphocyte antigen 4 (CTLA-4, B and T Lymphocyte Attenuator (BTLA; CD272), T cell Immunoglobulin and Mucin domain-3 (Tim-3), Lymphocyte Activation Gene-3 (Lag-3; CD223), and others are often referred to as a checkpoint regulators. They act as molecular “gatekeepers” that allow extracellular information to dictate whether cell cycle progression and other intracellular signaling processes should proceed.

In some embodiments, an immune checkpoint inhibitor is an antibody to PD-1. PD-1 binds to the programmed cell death 1 receptor (PD-1) to prevent the receptor from binding to the inhibitory ligand PDL-1, thus overriding the ability of tumors to suppress the host anti-tumor immune response.

In some embodiments, the checkpoint inhibitor is a biologic therapeutic or a small molecule. In some embodiments, the checkpoint inhibitor is a monoclonal antibody, a humanized antibody, a fully human antibody, a fusion protein or a combination thereof. In some embodiments, the checkpoint inhibitor inhibits a checkpoint protein selected from CTLA-4, PDL1, PDL2, PDl, B7-H3, B7-H4, BTLA, HVEM, TIM3, GAL9, LAG3, VISTA, KIR, 2B4, CD160, CGEN-15049, CHK1, CHK2, A2aR, B-7 family ligands or a combination thereof. In some embodiments, the checkpoint inhibitor interacts with a ligand of a checkpoint protein selected from CTLA-4, PDL1, PDL2, PDl, B7-H3, B7-H4, BTLA, HVEM, TIM3, GAL9, LAG3, VISTA, KIR, 2B4, CD160, CGEN-15049, CHK1, CHK2, A2aR, B-7 family ligands or a combination thereof. In some embodiments, the checkpoint inhibitor is an immunostimulatory agent, a T cell growth factor, an interleukin, an antibody, a vaccine or a combination thereof. In some embodiments, the interleukin is IL-7 or IL-15. In some embodiments, the interleukin is glycosylated IL-7. In an additional aspect, the vaccine is a dendritic cell (DC) vaccine.

Checkpoint inhibitors include any agent that blocks or inhibits in a statistically significant manner, the inhibitory pathways of the immune system. Such inhibitors can include small molecule inhibitors or can include antibodies, or antigen binding fragments thereof, that bind to and block or inhibit immune checkpoint receptors or antibodies that bind to and block or inhibit immune checkpoint receptor ligands. Illustrative checkpoint molecules that can be targeted for blocking or inhibition include, but are not limited to, CTLA-4, PDL1, PDL2, PD1, B7-H3, B7-H4, BTLA, HVEM, GAL9, LAG3, TIM3, VISTA, KIR, 2B4 (belongs to the CD2 family of molecules and is expressed on all NK, γδ, and memory CD8⁺ (αβ) T cells), CD160 (also referred to as BY55), CGEN-15049, CHK 1 and CHK2 kinases, A2aR, and various B-7 family ligands. B7 family ligands include, but are not limited to, B7-1, B7-2, B7-DC, B7-H1, B7-H2, B7-H3, B7-H4, B7-H5, B7-H6 and B7-H7. Checkpoint inhibitors include antibodies, or antigen binding fragments thereof, other binding proteins, biologic therapeutics, or small molecules, that bind to and block or inhibit the activity of one or more of CTLA-4, PDL1, PDL2, PD1, BTLA, HVEM, TIM3, GAL9, LAG3, VISTA, KIR, 2B4, CD 160 and CGEN-15049. Illustrative immune checkpoint inhibitors include, but are not limited to, Tremelimumab (CTLA-4 blocking antibody), anti-OX40, PD-L1 monoclonal Antibody (Anti-B7-H1; MED14736), MK-3475 (PD-1 blocker), Nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody), and ipilimumab (anti-CTLA-4 checkpoint inhibitor). Checkpoint protein ligands include, but are not limited to PD-L1, PD-L2, B7-H3, B7-H4, CD28, CD86 and TIM-3.

In certain embodiments, the immune checkpoint inhibitor is selected from a PD-1 antagonist, a PD-L1 antagonist, and a CTLA-4 antagonist. In some embodiments, the checkpoint inhibitor is selected from the group consisting of nivolumab (OPDIVO®), ipilimumab (YERVOY®), and pembrolizumab (KEYTRUDA®). In some embodiments, the checkpoint inhibitor is selected from nivolumab (anti-PD-1 antibody, OPDIVO®, Bristol-Myers Squibb); pembrolizumab (anti-PD-1 antibody, KEYTRUDA®, Merck); ipilimumab (anti-CTLA-4 antibody, YERVOY®, Bristol-Myers Squibb); durvalumab (anti-PD-L1 antibody, IMFINZI®, AstraZeneca); and atezolizumab (anti-PD-L1 antibody, TECENTRIQ®, Genentech).

In some embodiments, the checkpoint inhibitor is selected from the group consisting of lambrolizumab (MK-3475), nivolumab (BMS-936558), pidilizumab (CT-011), AMP-224, MDX-1105, MEDI4736, MPDL3280A, BMS-936559, ipilimumab, lirlumab, IPH2101, pembrolizumab (KEYTRUDA®), and tremelimumab.

In some embodiments, an immune checkpoint inhibitor is REGN2810 (Regeneron), an anti-PD-1 antibody tested in patients with basal cell carcinoma (NCT03132636); NSCLC (NCT03088540); cutaneous squamous cell carcinoma (NCT02760498); lymphoma (NCT02651662); and melanoma (NCT03002376); pidilizumab (CureTech), also known as CT-011, an antibody that binds to PD-1, in clinical trials for diffuse large B-cell lymphoma and multiple myeloma; avelumab (BAVENCIO®, Pfizer/Merck KGaA), also known as MSB0010718C), a fully human IgGI anti-PD-L1 antibody, in clinical trials for non-small cell lung cancer, Merkel cell carcinoma, mesothelioma, solid tumors, renal cancer, ovarian cancer, bladder cancer, head and neck cancer, and gastric cancer; or PDR001 (Novartis), an inhibitory antibody that binds to PD-1, in clinical trials for non-small cell lung cancer, melanoma, triple negative breast cancer and advanced or metastatic solid tumors. Tremelimumab (CP-675,206; Astrazeneca) is a fully human monoclonal antibody against CTLA-4 that has been in studied in clinical trials for a number of indications, including: mesothelioma, colorectal cancer, kidney cancer, breast cancer, lung cancer and non-small cell lung cancer, pancreatic ductal adenocarcinoma, pancreatic cancer, germ cell cancer, squamous cell cancer of the head and neck, hepatocellular carcinoma, prostate cancer, endometrial cancer, metastatic cancer in the liver, liver cancer, large B-cell lymphoma, ovarian cancer, cervical cancer, metastatic anaplastic thyroid cancer, urothelial cancer, fallopian tube cancer, multiple myeloma, bladder cancer, soft tissue sarcoma, and melanoma. AGEN-1884 (Agenus) is an anti-CTLA4 antibody that is being studied in Phase 1 clinical trials for advanced solid tumors (NCT02694822).

In some embodiments, a checkpoint inhibitor is an inhibitor of T-cell immunoglobulin mucin containing protein-3 (TIM-3). TIM-3 inhibitors that may be used in the present invention include TSR-022, LY3321367 and MBG453. TSR-022 (Tesaro) is an anti-TIM-3 antibody which is being studied in solid tumors (NCT02817633). LY3321367 (Eli Lilly) is an anti-TIM-3 antibody which is being studied in solid tumors (NCT03099109). MBG453 (Novartis) is an anti-TIM-3 antibody which is being studied in advanced malignancies (NCT02608268).

In some embodiments, a checkpoint inhibitor is an inhibitor of T cell immunoreceptor with Ig and ITIM domains, or TIGIT, an immune receptor on certain T cells and NK cells. TIGIT inhibitors that may be used in the present invention include BMS-986207 (Bristol-Myers Squibb), an anti-TIGIT monoclonal antibody (NCT02913313); OMP-313M32 (Oncomed); and anti-TIGIT monoclonal antibody (NCT03119428).

In some embodiments, a checkpoint inhibitor is an inhibitor of Lymphocyte Activation Gene-3 (LAG-3). LAG-3 inhibitors that may be used in the present invention include BMS-986016 and REGN3767 and IMP321. BMS-986016 (Bristol-Myers Squibb), an anti-LAG-3 antibody, is being studied in glioblastoma and gliosarcoma (NCT02658981). REGN3767 (Regeneron), is also an anti-LAG-3 antibody, and is being studied in malignancies (NCT03005782). IMP321 (Immutep S.A.) is an LAG-3-Ig fusion protein, being studied in melanoma (NCT02676869); adenocarcinoma (NCT02614833); and metastatic breast cancer (NCT00349934).

Checkpoint inhibitors that can be used in the present invention include OX40 agonists. OX40 agonists that are being studied in clinical trials include PF-04518600/PF-8600 (Pfizer), an agonistic anti-OX40 antibody, in metastatic kidney cancer (NCT03092856) and advanced cancers and neoplasms (NCT02554812; NCT05082566); GSK3174998 (Merck), an agonistic anti-OX40 antibody, in Phase 1 cancer trials (NCT02528357); MED10562 (Medimmune/AstraZeneca), an agonistic anti-OX40 antibody, in advanced solid tumors (NCT02318394 and NCT02705482); MED16469, an agonistic anti-OX40 antibody (Medimmune/AstraZeneca), in patients with colorectal cancer (NCT02559024), breast cancer (NCT01862900), head and neck cancer (NCT02274155) and metastatic prostate cancer (NCT01303705); and BMS-986178 (Bristol-Myers Squibb) an agonistic anti-OX40 antibody, in advanced cancers (NCT02737475).

Checkpoint inhibitors that can be used in the present invention include CD137 (also called 4-1BB) agonists. CD137 agonists that are being studied in clinical trials include utomilumab (PF-05082566, Pfizer) an agonistic anti-CD137 antibody, in diffuse large B-cell lymphoma (NCT02951156) and in advanced cancers and neoplasms (NCT02554812 and NCT05082566); urelumab (BMS-663513, Bristol-Myers Squibb), an agonistic anti-CD137 antibody, in melanoma and skin cancer (NCT02652455) and glioblastoma and gliosarcoma (NCT02658981); and CTX-471 (Compass Therapeutics), an agonistic anti-CD137 antibody in metastatic or locally advanced malignancies (NCT03881488).

Checkpoint inhibitors that can be used in the present invention include CD27 agonists. CD27 agonists that are being studied in clinical trials include varlilumab (CDX-1127, Celldex Therapeutics) an agonistic anti-CD27 antibody, in squamous cell head and neck cancer, ovarian carcinoma, colorectal cancer, renal cell cancer, and glioblastoma (NCT02335918); lymphomas (NCT01460134); and glioma and astrocytoma (NCT02924038).

Checkpoint inhibitors that can be used in the present invention include glucocorticoid-induced tumor necrosis factor receptor (GITR) agonists. GITR agonists that are being studied in clinical trials include TRX518 (Leap Therapeutics), an agonistic anti-GITR antibody, in malignant melanoma and other malignant solid tumors (NCT01239134 and NCT02628574); GWN323 (Novartis), an agonistic anti-GITR antibody, in solid tumors and lymphoma (NCT 02740270); INCAGN01876 (Incyte/Agenus), an agonistic anti-GITR antibody, in advanced cancers (NCT02697591 and NCT03126110); MK-4166 (Merck), an agonistic anti-GITR antibody, in solid tumors (NCT02132754) and MEDI1873 (Medimmune/AstraZeneca), an agonistic hexameric GITR-ligand molecule with a human IgG1 Fc domain, in advanced solid tumors (NCT02583165).

Checkpoint inhibitors that can be used in the present invention include inducible T-cell co-stimulator (ICOS, also known as CD278) agonists. ICOS agonists that are being studied in clinical trials include MEDI-570 (Medimmune), an agonistic anti-ICOS antibody, in lymphomas (NCT02520791); GSK3359609 (Merck), an agonistic anti-ICOS antibody, in Phase 1 (NCT02723955); JTX-2011 (Jounce Therapeutics), an agonistic anti-ICOS antibody, in Phase 1 (NCT02904226).

Checkpoint inhibitors that can be used in the present invention include killer IgG-like receptor (KIR) inhibitors. KIR inhibitors that are being studied in clinical trials include lirilumab (IPH2102/BMS-986015, Innate Pharma/Bristol-Myers Squibb), an anti-KIR antibody, in leukemias (NCT01687387, NCT02399917, NCT02481297, NCT02599649), multiple myeloma (NCT02252263), and lymphoma (NCT01592370); IPH2101 (1-7F9, Innate Pharma) in myeloma (NCT01222286 and NCT01217203); and IPH4102 (Innate Pharma), an anti-KIR antibody that binds to three domains of the long cytoplasmic tail (KIR3DL2), in lymphoma (NCT02593045).

Checkpoint inhibitors that can be used in the present invention include CD47 inhibitors of interaction between CD47 and signal regulatory protein alpha (SIRPa). CD47/SIRPa inhibitors that are being studied in clinical trials include ALX-148 (Alexo Therapeutics), an antagonistic variant of (SIRPa) that binds to CD47 and prevents CD47/SIRPa-mediated signaling, in phase 1 (NCT03013218); TTI-621 (SIRPa-Fc, Trillium Therapeutics), a soluble recombinant fusion protein created by linking the N-terminal CD47-binding domain of SIRPa with the Fc domain of human IgGI, acts by binding human CD47, and preventing it from delivering its “do not eat” signal to macrophages, is in clinical trials in Phase 1 (NCT02890368 and NCT02663518); CC-90002 (Celgene), an anti-CD47 antibody, in leukemias (NCT02641002); and Hu5F9-G4 (Forty Seven, Inc.), in colorectal neoplasms and solid tumors (NCT02953782), acute myeloid leukemia (NCT02678338) and lymphoma (NCT02953509).

Checkpoint inhibitors that can be used in the present invention include CD73 inhibitors. CD73 inhibitors that are being studied in clinical trials include MED19447 (Medimmune), an anti-CD73 antibody, in solid tumors (NCT02503774); and BMS-986179 (Bristol-Myers Squibb), an anti-CD73 antibody, in solid tumors (NCT02754141).

Checkpoint inhibitors that can be used in the present invention include agonists of stimulator of interferon genes protein (STING, also known as transmembrane protein 173, or TMEM173). Agonists of STING that are being studied in clinical trials include MK-1454 (Merck), an agonistic synthetic cyclic dinucleotide, in lymphoma (NCT03010176); and ADU-S100 (MIW815, Aduro Biotech/Novartis), an agonistic synthetic cyclic dinucleotide, in Phase 1 (NCT02675439 and NCT03172936).

Checkpoint inhibitors that can be used in the present invention include CSF1R inhibitors. CSF1R inhibitors that are being studied in clinical trials include pexidartinib (PLX3397, Plexxikon), a CSF1R small molecule inhibitor, in colorectal cancer, pancreatic cancer, metastatic and advanced cancers (NCT02777710) and melanoma, non-small cell lung cancer, squamous cell head and neck cancer, gastrointestinal stromal tumor (GIST) and ovarian cancer (NCT02452424); and IMC-CS4 (LY3022855, Lilly), an anti-CSF-1R antibody, in pancreatic cancer (NCT03153410), melanoma (NCT03101254), and solid tumors (NCT02718911); and BLZ945 (4-[2((1R,2R)-2-hydroxycyclohexylamino)-benzothiazol-6-yloxyl]-pyridine-2-carboxylic acid methylamide, Novartis), an orally available inhibitor of CSF1R, in advanced solid tumors (NCT02829723).

Checkpoint inhibitors that can be used in the present invention include NKG2A receptor inhibitors. NKG2A receptor inhibitors that are being studied in clinical trials include monalizumab (IPH2201, Innate Pharma), an anti-NKG2A antibody, in head and neck neoplasms (NCT02643550) and chronic lymphocytic leukemia (NCT02557516).

In some embodiments, the immune checkpoint inhibitor is selected from nivolumab, pembrolizumab, ipilimumab, avelumab, durvalumab, atezolizumab, or pidilizumab.

A compound of the current invention may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current invention can be administered alone or in combination with one or more other therapeutic compounds, possible combination therapy taking the form of fixed combinations or the administration of a compound of the invention and one or more other therapeutic compounds being staggered or given independently of one another, or the combined administration of fixed combinations and one or more other therapeutic compounds. A compound of the current invention can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Those additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a compound of the current invention, an additional therapeutic agent, and a pharmaceutically acceptable carrier, adjuvant, or vehicle.

The amount of both an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions of this invention should be formulated so that a dosage of between 0.01-100 mg/kg body weight/day of an inventive compound can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent. In such compositions a dosage of between 0.01-1,000 g/kg body weight/day of the additional therapeutic agent can be administered.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

The compounds of this invention, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this invention are another embodiment of the present invention.

### EXEMPLIFICATION

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein. Additional compounds of the invention were prepared by methods substantially similar to those described herein in the Examples and methods known to one skilled in the art.

### Synthesis of Intermediates

### Synthesis of 2′-(4-Methyl-4H-1,2,4-triazol-3-yl)-[1,1′-biphenyl]-3-carbaldehyde (Intermediate A-7)

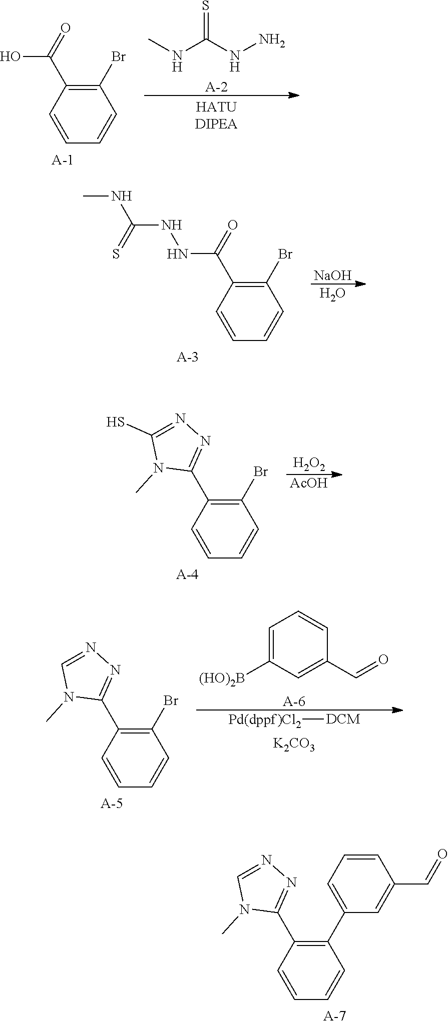

Step 1: 2-(2-Bromobenzoyl)-N-methylhydrazine-1-carbothioamide (A-3). To a stirred solution of 2-bromobenzoic acid (A-1) (10.2 g, 1 Eq, 50.6 mmol) and 1-amino-3-methyl-thiourea (A-2) (5.32 g, 1.0 Eq, 50.6 mmol) in DMF (100 mL) was added HATU (23.1 g, 1.2 Eq, 60.7 mmol)) in portions at 0° C. Then DIPEA (32.7 g, 44.1 mL, 5.0 Eq, 253 mmol) was added at the same temperature under nitrogen. The resulting mixture was stirred for 16 h at rt. Water (200 mL) was added and the aqueous layer was extracted with EtOAc (3×200 mL). The combined organic layers were washed with brine (3×200 mL) and dried (Na₂SO₄), then concentrated in vacuo to afford crude sub-title compound (A-3) (15.3 g, 37 mmol, 73%, 70% Purity) as a yellow oil, which was taken forward without purification. m/z 286/288 (M−H)⁻ (ES−).

Step 2: 5-(2-Bromophenyl)-4-methyl-4H-1,2,4-triazole-3-thiol (A-4). A solution of the product from step 1, above (A-3) (15.3 g, 70% Wt, 1.0 Eq, 37.2 mmol) in sodium hydroxide (4.31 g, 108 mL, 1.0 molar, 2.90 Eq, 108 mmol) was stirred overnight at 50° C. Water (300 mL) was added, the mixture was acidified to pH5 with HCl (aq. 1 M) at 0° C. The aqueous layer was extracted with EtOAc (3×200 mL). The organic extracts were combined, dried (Na₂SO₄), and concentrated in vacuo to afford the sub-title compound (A-4) (6.55 g, 21 mmol, 55%, 85% Purity) as a yellow oil. m/z 270/272 (M+H)⁺ (ES+).

Step 3: 3-(2-Bromophenyl)-4-methyl-4H-1,2,4-triazole (A-5). To a stirred mixture of the product from step 2, above (A-4) (6.55 g, 30% Wt, 1 Eq, 7.27 mmol) in DCM (80 mL) was added AcOH (874 mg, 833 μL, 2 Eq, 14.5 mmol) dropwise at 0° C. To the above mixture was added H₂O₂ (30 wt %) (4.95 g, 4.46 mL, 30% Wt, 6 Eq, 43.6 mmol) drop-wise at 0° C. The resulting mixture was stirred for 1.5 h at room temperature. The resulting mixture was diluted with water (200 mL). The mixture was basified to pH 8 with saturated NaHCO₃ aqueous solution. The aqueous layer was extracted with DCM (3×200 mL). The organic phase was dried (Na₂SO₄), then concentrated in vacuo. The crude product was purified by chromatography on silica gel (80 g cartridge, 0-10% MeOH/DCM) to afford impure material which was triturated with DCM (10 mL) and the precipitate filtered. The filtrate was concentrated in vacuo and was purified by chromatography on RP Flash C18 (40 g cartridge, 0-100% MeCN/10 mM ammonium bicarbonate) to afford the sub-title compound (A-5) (400 mg, 1.5 mmol, 21%, 90% Purity) as a pale tan solid. m/z 238.1/240.1 (M+H)⁺ (ES+). ¹H NMR (400 MHz, DMSO-d6) δ 8.63 (s, 1H), 7.86-7.81 (m, 1H), 7.59-7.50 (m, 3H), 3.48 (s, 3H).

Step 4: 2′-(4-Methyl-4H-1,2,4-triazol-3-yl)-[1,1′-biphenyl]-3-carbaldehyde (A-7). To a solution of (3-formylphenyl)boronic acid (A-6) (151 mg, 1.2 Eq, 1.01 mmol) and the product from step 3 above (200 mg, 1 Eq, 840 μmol) in degassed 1,4-Dioxane (4 mL) and water (1 mL) were successively added Pd(dppf)Cl₂·DCM (68.6 mg, 0.1 Eq, 84.0 μmol) and K₂CO₃ (348 mg, 3 Eq, 2.52 mmol). This mixture was stirred at 90° C. for 3 h. The reaction mixture was diluted with EtOAc (25 mL), washed with sat. aq. sol. of NH₄Cl (5 mL), sat. aq. sol. of NaHCO₃ (5 mL) and brine (5 mL). The combined organic extracts were dried (MgSO₄), filtered and concentrated in vacuo. The crude product was purified by chromatography on silica gel (24 g cartridge, 0-40% MeOH/DCM) to afford the title compound (A-7) (161 mg, 0.55 mmol, 66%, 90% Purity) as a pale brown solid. m/z 264.4 (M+H)⁺ (ES+). ¹H NMR (400 MHz, DMSO-d6) δ 9.95 (s, 1H), 8.39 (s, 1H), 7.86-7.82 (m, 1H), 7.75-7.65 (m, 3H), 7.64-7.58 (m, 2H), 7.53 (t, J=7.7 Hz, 1H), 7.42-7.37 (m, 1H), 3.06 (s, 3H).

### Synthesis of 4′-Fluoro-2′-(4-methyl-4H-1,2,4-triazol-3-yl)-[1,1′-biphenyl]-3-carbaldehyde (Intermediate B-5)

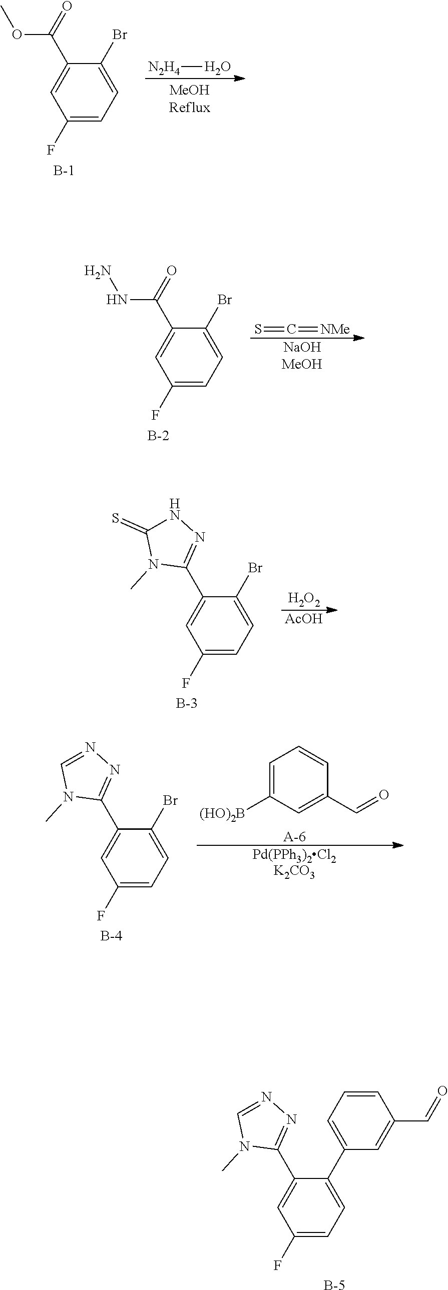

Step 1: 2-Bromo-5-fluorobenzohydrazide (B-2). To a solution of methyl 2-bromo-5-fluorobenzoate (B-1) (2.10 g, 1 Eq, 9.01 mmol) in MeOH (2 mL) was added hydrazine hydrate (64% hydrazine) (2.26 g, 2.21 mL, 64% Wt, 5 Eq, 45.1 mmol) at 0° C. The mixture was warmed to rt then heated to reflux for 2 h. The reaction mixture was cooled, allowing a precipitate to form. The residue was diluted with water and the precipitate filtered and dried in vacuo to afford the sub-title compound (B-2) (2.34 g, 9.0 mmol, 100%, 90% Purity) as a white solid. ¹H NMR (400 MHz, DMSO-d6) δ 9.62 (s, 1H), 7.72-7.66 (m, 1H), 7.30-7.21 (m, 2H), 4.48 (s, 2H).

Step 2: 5-(2-Bromo-5-fluorophenyl)-4-methyl-2,4-dihydro-3H-1,2,4-triazole-3-thione (B-3). A mixture of the product from step 1 above (B-2) (1.96 g, 1 Eq, 8.41 mmol) and isothiocyanatomethane (738 mg, 1.2 Eq, 10.1 mmol) in MeOH (20 mL) was stirred at rt under N₂ for 16 h. NaOH (1.35 g, 16.8 mL, 2 molar, 4 Eq, 33.6 mmol) was added and the reaction mixture was refluxed for 2 h. The reaction mixture was cooled to 0° C. and acidified with 1 M HCl. The resulting solution was extracted with DCM (3×20 mL), dried (phase separator) and concentrated to afford the sub-title compound (B-3) (1.95 g, 6.1 mmol, 72%, 90% Purity) as a yellow oil. ¹H NMR (400 MHz, DMSO-d6) δ 7.90 (dd, J=8.9, 5.1 Hz, 1H), 7.65 (dd, J=8.8, 3.1 Hz, 1H), 7.49 (td, J=8.7, 3.2 Hz, 1H), 3.26 (s, 3H). One exchangeable proton not observed.

Step 3: 3-(2-Bromo-5-fluorophenyl)-4-methyl-4H-1,2,4-triazole (B-4). To a solution of the product from step 2 above (B-3) (1.95 g, 90% Wt, 1 Eq, 6.12 mmol) in DCM (40 mL) and AcOH (11.0 g, 10.5 mL, 30 Eq, 184 mmol) was added H₂O₂ (30% in water) (6.94 g, 6.25 mL, 30% Wt, 10 Eq, 61.2 mmol) drop-wise with stirring at 0° C. The mixture was stirred at this temperature for 1 h then concentrated. The residue was dissolved in water, basified with NaOH (2 M) to pH 10, and extracted with EtOAc (2×40 mL). The organic extract was washed with brine (40 mL), dried (MgSO₄), filtered, and concentrated in vacuo. The crude product was purified by chromatography on silica gel (12 g cartridge, 0-10% MeOH/DCM) to afford the sub-title compound (B-4) (1.22 g, 4.3 mmol, 70%, 90% Purity) as a pale-yellow oil. m/z 256.8/258.0 (M+H)⁺ (ES+). ¹H NMR (400 MHz, DMSO-d6) δ 8.65 (s, 1H), 7.88 (dd, J=8.9, 5.2 Hz, 1H), 7.51 (dd, J=8.9, 3.1 Hz, 1H), 7.44 (td, J=8.6, 3.1 Hz, 1H), 3.55-3.46 (m, 3H).

Step 4: 4′-Fluoro-2′-(4-methyl-4H-1,2,4-triazol-3-yl)-[1,1′-biphenyl]-3-carbaldehyde (B-5). To a solution of (3-formylphenyl)boronic acid (A-6) (253 mg, 1.2 Eq, 1.69 mmol) and the product from step 3 above (B-4) (400 mg, 90% Wt, 1 Eq, 1.41 mmol) in degassed dioxane (9 mL) and water (4 mL) were successively added Pd(PPh₃)₂·Cl₂ (98.7 mg, 0.1 Eq, 141 μmol) and K₂CO₃ (583 mg, 3 Eq, 4.22 mmol) and stirred at 80° C. for 4 h. The reaction mixture was concentrated in vacuo and the crude product was purified by chromatography on RP Flash C18 (24 g cartridge, 0-100% MeCN/10 mM ammonium bicarbonate) to afford the title compound (B-5) (213.2 mg, 0.72 mmol, 51%, 95% Purity) as a pale yellow solid. m/z 282.4 (M+H)⁺ (ES+). ¹H NMR (400 MHz, DMSO-d6) δ 9.95 (s, 1H), 8.42 (s, 1H), 7.84 (dt, J=7.7, 1.4 Hz, 1H), 7.75-7.70 (m, 1H), 7.66 (t, J=1.8 Hz, 1H), 7.58 (td, J=8.6, 2.8 Hz, 1H), 7.55-7.49 (m, 2H), 7.39-7.34 (m, 1H), 3.12 (s, 3H).

### Synthesis of 4-Hydroxy-3-nitro-5-(trifluoromethyl)benzaldehyde (Intermediate C-2)

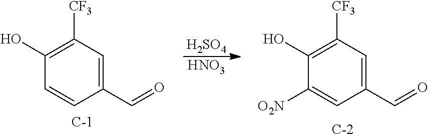

To a mixture of 4-hydroxy-3-(trifluoromethyl)benzaldehyde (C-1) (200 mg, 1 Eq, 1.05 mmol) suspended in conc. H₂SO₄ (3.61 g, 1.97 mL, 35 Eq, 36.8 mmol) was added HNO₃ (166 mg, 110 μL, 2.5 Eq, 2.63 mmol) slowly at 0° C. The resulting suspension was stirred at this temperature for 30 min. The mixture was poured into ice water (30 mL) and the precipitate was filtered and dried to afford the title compound (168 mg, 0.68 mmol, 65%, 95% Purity) as a tan solid. m/z 234.1 (M−H)⁻ (ES−). ¹H NMR (400 MHz, DMSO-d6) δ 9.76 (s, 1H), 8.52 (d, J=2.2 Hz, 1H), 8.14 (d, J=2.2 Hz, 1H). One exchangeable proton not observed.

### Synthesis of Methyl 3-amino-4-hydroxy-5-(trifluoromethyl)benzoate (Intermediate D-3)

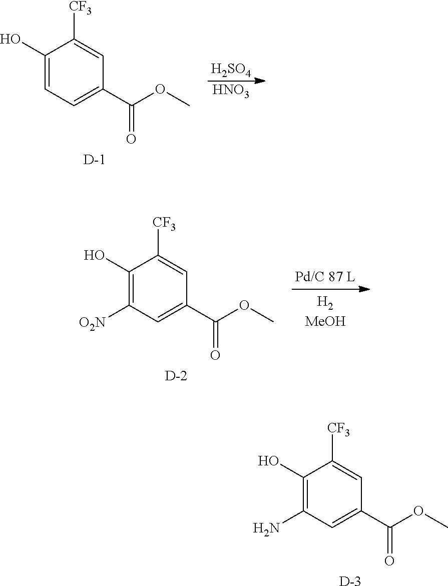

Step 1: Methyl 4-hydroxy-3-nitro-5-(trifluoromethyl)benzoate (D-2). To a mixture of methyl 4-hydroxy-3-(trifluoromethyl)benzoate (D-1) (1.00 g, 1 Eq, 4.54 mmol) suspended in conc. H₂SO₄ (15.6 g, 8.52 mL, 35 Eq, 159 mmol) was added HNO₃ (716 mg, 474 μL, 2.5 Eq, 11.4 mmol) slowly at 0° C. The resulting suspension was stirred at this temperature for 10 min. The mixture was poured into ice water (20 mL), the precipitate was filtered and dried to afford the sub-title compound (D-2) (1.09 g, 4.0 mmol, 88%, 97% Purity) as a tan solid. m/z 264.1 (M−H)⁻ (ES−). ¹H NMR (400 MHz, DMSO-d6) δ 8.60 (d, J=2.2 Hz, 1H), 8.25 (d, J=2.2 Hz, 1H), 3.87 (s, 3H). One exchangeable proton not observed.

Step 2: Methyl 3-amino-4-hydroxy-5-(trifluoromethyl)benzoate (D-3). To a solution of the product from step 1 above (D-2) (300 mg, 1 Eq, 1.13 mmol) in MeOH (5 mL) was added Pd/C 87 L (30 mg, 0.25 Eq, 0.28 mmol) and stirred at 45° C. with 5 atm of H₂ for 16 h. The catalyst was removed by filtration and the solvent removed in vacuo to afford the title compound (D-3) (260 mg, 1.1 mmol, 97%, 99% Purity) as a brown solid. m/z 236.2 (M+H)⁺ (ES+). ¹H NMR (400 MHz, DMSO-d6) δ 7.52 (d, J=2.1 Hz, 1H), 7.35 (d, J=2.1 Hz, 1H), 3.80 (s, 3H). Three exchangeable protons not observed.

### Synthesis of (4-Amino-3-nitro-5-(trifluoromethyl)phenyl)methanol (Intermediate E-3)

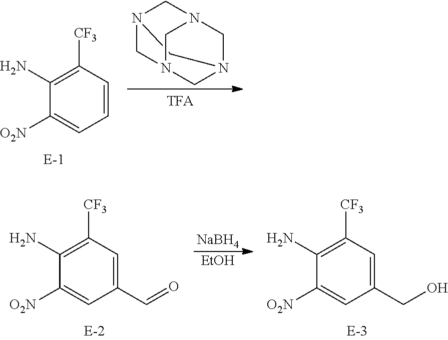

Step 1: 4-Amino-3-nitro-5-(trifluoromethyl)benzaldehyde (E-2). 2-Nitro-6-(trifluoromethyl)aniline (E-1) (2.0 g, 1 Eq, 9.7 mmol) and hexamethylenetetramine (1.4 g, 1 Eq, 9.7 mmol) were dissolved in TFA (30 g, 20 mL, 27 Eq, 0.26 mol) and heated to 70° C. for 16 h. The reaction mixture was cooled to rt, diluted with water, and extracted with DCM (3×20 mL). The organic extracts were combined, dried (phase separator) and concentrated in vacuo. The crude product was purified by chromatography on silica gel (24 g cartridge, 0-50% EtOAc/isohexane) to afford the sub-title compound (E-2) (1.12 g, 4.7 mmol, 48%, 98% Purity) as a bright orange solid. ¹H NMR (400 MHz, DMSO-d6) δ 9.87 (s, 1H), 8.83 (d, J=1.9 Hz, 1H), 8.26 (d, J=1.9 Hz, 1H), 8.05 (s, 2H).

Step 2: (4-Amino-3-nitro-5-(trifluoromethyl)phenyl)methanol (E-3). To a stirring solution of the product from step 1 above (E-2) (200 mg, 1 Eq, 854 μmol) in EtOH (5 mL) at rt was added NaBH₄ (64.6 mg, 2 Eq, 1.71 mmol) portion-wise and the reaction stirred at rt for 72 h. The reaction mixture was quenched with water and the solvent was removed in vacuo. The crude product was purified by chromatography on silica gel (40 g cartridge, 5-50% MeOH/DCM) to afford the title compound (E-3) (120 mg, 0.46 mmol, 54%, 90% Purity) as a dark orange oil. m/z 235.0 (M−H)⁻ (ES−) ¹H NMR (400 MHz, DMSO-d6) δ 8.26 (d, J=2.0 Hz, 1H), 7.82 (d, J=2.1 Hz, 1H), 7.28 (s, 2H), 5.36 (t, J=5.8 Hz, 1H), 4.45 (d, J=5.8 Hz, 2H).

### Synthesis of 3-(3-((4-Methyl-4H-1,2,4-triazol-3-yl)methyl)oxetan-3-yl)benzaldehyde (Intermediate AAA-10)

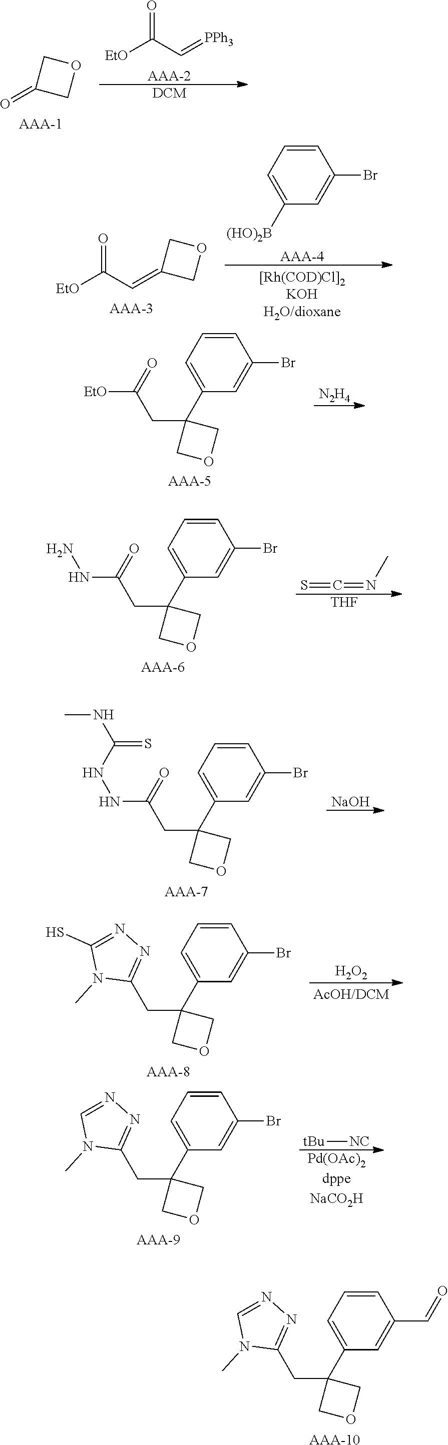

Step 1: Ethyl 2-(oxetan-3-ylidene) acetate (AAA-3). A solution of 3-oxetanone (14.40 g, 1 Eq, 200 mmol) in DCM for 2 h followed by the addition of ethyl-2-(triphenyl-lambda5-phosphanylidene)acetate (AAA-2) (70.00 g, 1 Eq, 200 mmol) at 0° C. The mixture was concentrated in vacuo. The residue was applied on a silica gel column chromatography with petroleum ether/EtOAc (5/1) to afford the sub-title compound (AAA-3) (19.2 g, 135 mmol, 67%) as a white solid. m/z 143.1 (M+H)⁺ (ES+).

Step 2: Ethyl 2-(3-(3-bromophenyl)oxetan-3-yl)acetate (AAA-5). To a stirred solution of the product from step 1 above (AAA-3) (10.00 g, 1 Eq, 70 mmol) and [Rh(COD)Cl]₂ (1.73 g, 0.05 Eq, 3.52 mmol) in 1,4-dioxane (500 mL), was added a solution of KOH (5.92 g, 105.519 mmol, 1.50 Eq) in water (50 mL) dropwise under nitrogen atmosphere. The resulting mixture was stirred for 30 min at rt under nitrogen atmosphere. To this was added 3-bromophenylboronic acid (AAA-4) (21.19 g, 1.5 Eq, 106 mmol). The resulting mixture was stirred for overnight at rt under nitrogen atmosphere. The reaction was quenched with NH₄Cl (aq.) at rt. The resulting mixture was diluted with water and extracted with EtOAc (3×1 L). The combined organic layers were washed with brine (2×2 L), dried (Na₂SO₄). After filtration, the filtrate was concentrated in vacuo. The crude product was purified by reverse flash column chromatography with the following conditions: Column, C18; mobile phase, Water (0.1% NH₄HCO₃) and ACN (0% ACN up to 60% in 20 min); Detector, UV 254/220 nm. The resulting mixture was concentrated to afford the sub-title compound (AAA-5) (6.0 g, 20 mmol, 29%) as a white solid. m/z 299.0 (M+H)⁺ (ES+).

Step 3: 2-(3-(3-Bromophenyl)oxetan-3-yl)acetohydrazide (AAA-6). To a solution of the product from step 2 above (AAA-5) (2.5 g, 1 Eq, 8.36 mmol) in EtOH (30 mL) and hydrazine hydrate (10 mL, 30% Wt). The resulting mixture was stirred overnight at 50° C. The mixture was allowed to cool down to rt. The resulting mixture was concentrated in vacuo to afford the sub-title compound (AAA-6) (2.2 g, 7.75 mmol, 92%) as a white solid. m/z 285.0 (M+H)⁺ (ES+).

Step 4: 2-(2-(3-(3-Bromophenyl)oxetan-3-yl)acetyl)-N-methylhydrazine-1-carbothioamide (AAA-7). To a solution of the product from step 3 above (AAA-6) (2.2 g, 1 Eq, 7.72 mmol) in THF (30 mL) was added methyl isothiocyanate (1.10 g, 2 Eq, 15.4 mmol). The resulting mixture was stirred overnight at 50° C. The mixture was allowed to cool down to rt. The resulting mixture was concentrated in vacuo to afford the sub-title compound (AAA-7) (2.2 g, 6.16 mmol, 80%) as a white solid. m/z 358.0 (M+H)⁺ (ES+).

Step 5: 5-((3-(3-Bromophenyl)oxetan-3-yl)methyl)-4-methyl-4H-1,2,4-triazole-3-thiol (AAA-8). The product from step 4 above (AAA-7) (2.2 g, 1 Eq, 6.15 mmol) was added in a solution of NaOH in water (12.3 mL, 1 molar, 2 Eq, 12.3 mmol). The resulting mixture was stirred overnight at rt. The pH of the solution was adjusted to 5 with HCl (aq., 1 M). The solids were collected by filtration. The resulting solid was washed with water (20 mL). The solid was dried to afford the sub-title compound (AAA-8) (1.5 g, 4.42 mmol, 71%) as a white solid. m/z 340.0 (M+H)⁺ (ES+).

Step 6: 3-((3-(3-Bromophenyl)oxetan-3-yl)methyl)-4-methyl-4H-1,2,4-triazole (AAA-9). To a solution of the product from step 5 above (AAA-8) (1.5 g, 1 Eq, 4.42 mmol) in DCM (10 mL) was added dropwise hydrogen peroxide (10 mL, 30% Wt) at 0° C. The resulting solution was stirred for 2 h at rt. The pH of the solution was adjusted to 8 with sat. Na₂CO₃ (aq.). The resulting mixture was diluted with water and extracted with EtOAc (3×100 mL). The combined organic layers were washed with brine (2×100 mL), dried (Na₂SO₄). After filtration, the filtrate was concentrated in vacuo. The crude product was purified by reverse flash column chromatography with the following conditions: Column, C18; mobile phase, Water (0.1% TFA) and ACN (0% ACN up to 25% in 20 min); Detector, UV 254/220 nm to afford the sub-title compound (AAA-9) (1.0 g, 3.26 mmol, 74%) as a white solid. m/z 308.0 (M+H)⁺ (ES+)

Step 7: 3-(3-((4-Methyl-4H-1,2,4-triazol-3-yl)methyl)oxetan-3-yl)benzaldehyde (AAA-10). To a stirred solution of the product from step 6 above (AAA-9) (100.00 mg, 1 Eq, 324 μmol) and tert-butyl isocyanide (32 mg, 1.2 Eq, 389 μmol) in DMSO (10 mL) was added potassium formate (30 mg, 2 Eq, 649 μmol), dppf (36 mg, 0.2 Eq, 65 μmol) and Pd(OAc)₂ (7 mg, 0.1 Eq, 32 μmol) at rt under nitrogen atmosphere. The resulting mixture was stirred for 1.5 h at 120° C. under nitrogen atmosphere. The mixture was allowed to cool down to rt. The residue was purified by Prep-TLC with DCM/MeOH (15/1) to afford the title compound (AAA-10) (80 mg, 311 μmol, 96%) as a yellow solid. m/z 258.1 (M+H)⁺ (ES+)

### Synthesis of 6-(Ethylamino)-4-(2-(4-methyl-4H-1,2,4-triazol-3-yl)phenyl)picolinic Acid (Intermediate AS-10)

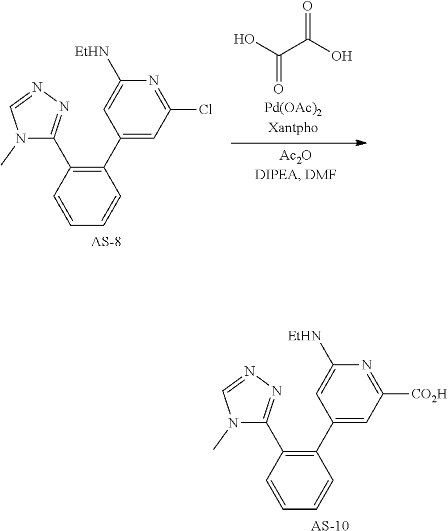

To a stirred solution of intermediate (AS-8) (900 mg, 1 Eq, 2.87 mmol), DIPEA (556 mg, 1.5 Eq, 4.30 mmol) and oxalic acid (387 mg, 1.5 Eq, 4.30 mmol) in DMF (50 mL) were added acetic anhydride (439 mg, 1.5 Eq, 4.30 mmol), XantPhos (332 mg, 0.2 Eq, 574 μmol) and Pd(OAc)₂ (64 mg, 0.1 Eq, 287 μmol) at rt under nitrogen atmosphere. The resulting mixture was stirred for overnight at 100° C. under nitrogen atmosphere. The mixture was allowed to cool down to rt. The crude product was purified by reverse flash column chromatography with the following conditions: Column, C18; mobile phase, Water (0.1% NH₄HCO₃) and MeCN (0% MeCN up to 15% in 20 min); Detector, UV 254/220 nm. This resulted in the sub-title compound (AS-10) (600 mg, 1.86 mmol, 60%) as an off-white solid. m/z 324.1 (M+H)⁺ (ES+).

### Synthesis of 2′-(4-Methyl-4H-1,2,4-triazol-3-yl)-[1,1′-biphenyl]-3-carbaldehyde (Intermediate A-7)

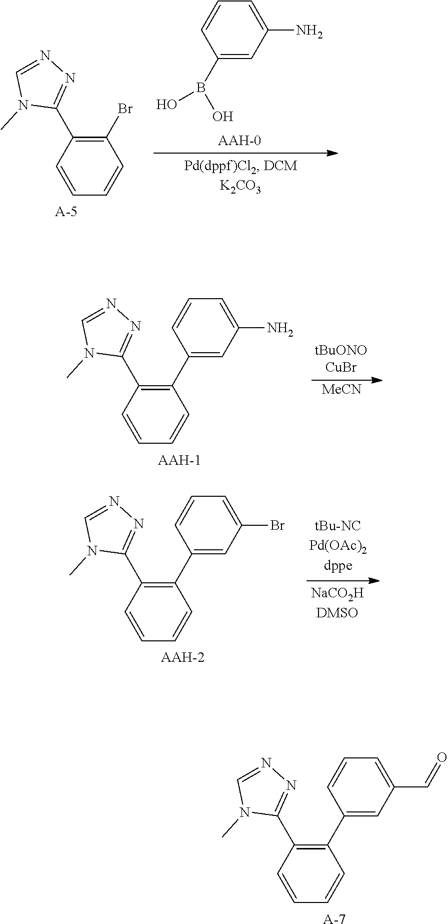

Step 1: 3-(3′-Bromo-[1,1′-biphenyl]-2-yl)-4-methyl-4H-1,2,4-triazole (AAH-1). To a stirred mixture of intermediate (A-5) (2.00 g, 1 Eq, 8.40 mmol), m-aminophenylboronic acid (AAH-0) (1.27 g, 1.1 Eq, 9.27 mmol) and K₂CO₃ (3.48 g, 3 Eq, 25.2 mmol) in 1,4-dioxane (10 mL) and water (2 mL) were added Pd(dppf)Cl₂-DCM (610 g, 0.1 Eq, 834 μmol) at rt under hydrogen atmosphere. The resulting mixture was stirred for 3 h at 80° C. under nitrogen atmosphere. The residue was cooled to rt and purified by reverse flash column chromatography with the following conditions: Column, C18; mobile phase, Water (0.1% NH₄HCO₃) and MeCN (0% MeCN up to 60% in 20 min); Detector, UV 254/220 nm. This resulted in the sub-title compound (AAH-1) (1.5 g, 5.99 mmol, 71%) as a yellow solid. m/z 251.1 (M+H)⁺ (ES+).

Step 2: 3-(3′-Bromo-[1,1′-biphenyl]-2-yl)-4-methyl-4H-1,2,4-triazole (AAH-2). To a stirred mixture of the product from step 1 (AAH-1) (250 mg, 1 Eq, 990 μmol) and tert-Butyl Isocyanide (206 mg, 2 Eq, 1.99 mmol) in MeCN (3 mL) was added CuBr (172 mg, 1.2 Eq, 1.19 mmol) at rt under nitrogen atmosphere. The resulting mixture was stirred for overnight at room temperature under nitrogen atmosphere. The resulting mixture was diluted with water and extracted with DCM (3×50 mL). The combined organic layers were washed with brine (2×50 mL), dried (Na₂SO₄). After filtration, the filtrate was concentrated in vacuo. The crude product was purified by reverse flash column chromatography with the following conditions: Column, C18; mobile phase, Water (0.1% NH₄HCO₃) and MeCN (20% MeCN up to 60% in 20 min); Detector, UV 254/220 nm. The product-containing fractions were combined and evaporated to afford the sub-title compound (AAH-2) (297 mg, 949 μmol, 70%) as a brown solid. m/z 314.0 (M+H)⁺ (ES+).

Step 3: 2′-(4-Methyl-4H-1,2,4-triazol-3-yl)-[1,1′-biphenyl]-3-carbaldehyde (A-7). To a stirred mixture of the product from step 1 above (AAH-2) (265 mg, 1 Eq, 840 μmol) and tert-butyl isocyanide (84 mg, 1.2 Eq, 1.01 mmol) in DMSO (3 mL) were added potassium formate (142 mg, 2 Eq, 1.68 mmol) and Pd(OAc)₂ (19 mg, 0.1 Eq, 80 μmol) and dppe (67 mg, 0.2 Eq, 160 μmol) at rt under nitrogen atmosphere. The resulting mixture was diluted with water and extracted with DCM (3×20 mL). The combined organic layers were washed with brine (2×20 mL), dried (Na₂SO₄). After filtration, the filtrate was concentrated in vacuo. The crude product was purified by reverse flash column chromatography with the following conditions: Column, C18; mobile phase, Water (0.1% NH₄HCO₃) and ACN (10% ACN up to 50% in 20 min); Detector, UV 254/220 nm. The product-containing fractions were combined and evaporated to afford the title compound (A-7) (83 mg, 316 μmol, 37%) as a white solid. m/z 264.1 (M+H)⁺ (ES+).

### Synthesis of 6-(Ethylamino)-4-(1-methyl-4-(4-methyl-4H-1,2,4-triazol-3-yl)-1H-pyrazol-5-yl)picolinic Acid (Intermediate AAC-3)

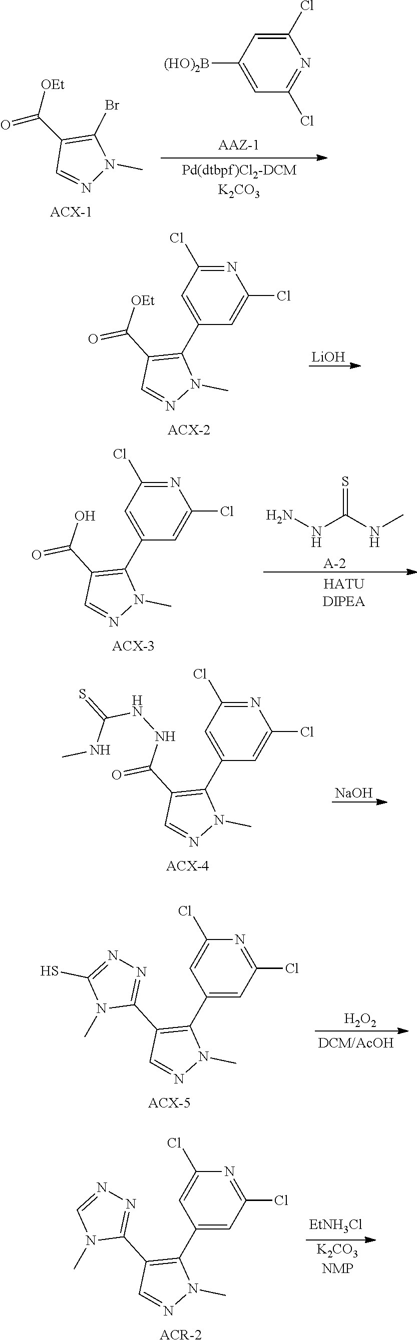

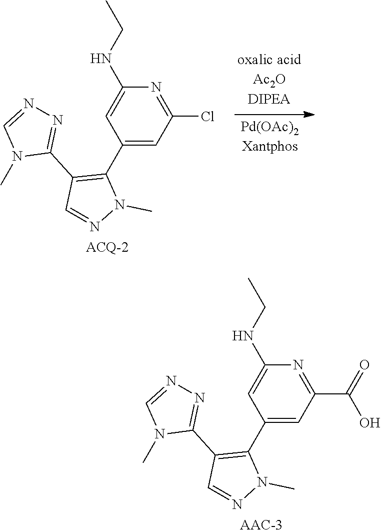

Step 1: Ethyl 5-(2,6-dichloropyridin-4-yl)-1-methyl-1H-pyrazole-4-carboxylate (ACX-2). To a stirred solution of intermediate (ACX-1) (1.00 g, 1 Eq, 4.29 mmol) and 2,6-dichloropyridin-4-ylboronic acid (AAZ-1) (823 mg, 1 Eq, 4.29 mmol) and K₂CO₃ (1.77 g, 3 Eq, 12.9 mmol) in 1,4-dioxane (37.5 mL) and water (7.5 mL) was added Pd(dtbpf)Cl₂·DCM (280 mg, 0.1 Eq, 420 μmol) at rt under nitrogen atmosphere. The resulting mixture was stirred overnight at 60° C. under nitrogen atmosphere. The mixture was cooled to rt and concentrated in vacuo. The residue was purified by silica gel column chromatography, eluted with petroleum ether/EtOAc (5/1) to afford the sub-title compound (ACX-2) (550 mg, 18.3 mmol, 55%) as a yellow solid. m/z 300.0/302.0 (M+H)⁺ (ES+).

Step 2: 5-(2,6-Dichloropyridin-4-yl)-1-methyl-1H-pyrazole-4-carboxylic acid (ACX-3). To a solution of the product from step 1 above (ACX-2) (550 mg, 1 Eq, 1.83 mmol) in THF (20 mL) and water (5 mL) was added LiOH (439 mg, 10 Eq, 18.3 mmol) at rt. The resulting mixture was stirred overnight at 60° C. then cooled to rt. The mixture was acidified to pH 3 with HCl (aq., 1 M). The resulting mixture was diluted with water and extracted with EtOAc (3×50 mL). The combined organic layers were washed with brine (2×50 mL), dried (Na₂SO₄), filtered and concentrated in vacuo. This resulted in the sub-title compound (ACX-3) (850 mg, 3.12 mmol, crude) as a yellow solid. m/z 272.1/274.1 (M+H)⁺ (ES+).

Step 3: 2-(5-(2,6-Dichloropyridin-4-yl)-1-methyl-1H-pyrazole-4-carbonyl)-N-methylhydrazine-1-carbothioamide (ACX-4). To a stirred solution of the product from step 2 above (ACX-3) (850 mg, 1 Eq, 3.12 mmol) and DIPEA (1.21 g, 3 Eq, 9.37 mmol) in DMF (10 mL) were added 4-methyl-3-thiosemicarbazide (A-2) (329 mg, 1 Eq, 3.12 mmol) and HATU (1.19 g, 1 Eq, 3.12 mmol) at 0° C. The resulting mixture was stirred for 2 h at rt and the crude product used directly in next step without any further purification. m/z 359.2/361.2 (M+H)⁺ (ES+).

Step 4: 5-(5-(2,6-Dichloropyridin-4-yl)-1-methyl-1H-pyrazol-4-yl)-4-methyl-4H-1,2,4-triazole-3-thiol (ACX-5). To the reaction mixture from step 3 above (ACX-4) was added NaOH (aq., 1M) (12.5 mL) at 0° C. The resulting mixture was stirred overnight at rt. The crude product was purified by reverse flash column chromatography with the following conditions: Column, C18; mobile phase, Water (0.1% NH₄HCO₃) and ACN (36% ACN up to 48% in 10 min); Detector, UV 254/220 nm. The product-containing fractions were combined and concentrated in vacuo. This resulted in the sub-title compound (ACX-5) (620 mg, 18.1 mmol, 92%) as a white solid. m/z 341.2/343.2 (M+H)⁺ (ES+).

Step 5: 2,6-Dichloro-4-(1-methyl-4-(4-methyl-4H-1,2,4-triazol-3-yl)-1H-pyrazol-5-yl) pyridine (ACR-2). To a solution of the product from step 4 above (ACX-5) (620 mg, 1 Eq, 1.81 mmol) in DCM (10 mL) was added acetic acid (218.24 mg, 2 Eq, 3.63 mmol) and hydrogen peroxide (309 mg, 30% Wt, 5 Eq, 9.08 mmol) at 0° C. The resulting mixture was stirred for 1 h at rt then the mixture basified to pH 8 with sat. NaHCO₃ (aq., 1M) solution. The resulting mixture was diluted with water and extracted with DCM (3×80 mL). The combined organic layers were washed with brine (2×80 mL), dried (Na₂SO₄), filtered and concentrated in vacuo. This resulted in the sub-title compound (ACR-2) (360 mg, 1.17 mmol, 58%) as a white solid. m/z 309.1/311.1 (M+H)⁺ (ES+).

Step 6: 6-Chloro-N-ethyl-4-(1-methyl-4-(4-methyl-4H-1,2,4-triazol-3-yl)-1H-pyrazol-5-yl)pyridin-2-amine (ACQ-2). To a stirred mixture of the product from step 5 above (ACR-2) (1.00 g, 1 Eq, 3.24 mmol) and ethylamine, HCl (2.64 g 10 Eq, 32.4 mmol) in NMP (25 mL) was added K₂CO₃ (4.47 g, 10 Eq, 32.4 mmol) at rt under nitrogen atmosphere. The resulting mixture was stirred for overnight at 100° C. under nitrogen atmosphere then cooled to rt. The crude product was purified by reverse flash column chromatography with the following conditions: Column, C18; mobile phase, Water (0.1% FA) and MeCN (10% MeCN up to 30% in 20 min); Detector, UV 254/220 nm to afford the sub-title compound (ACQ-2) (800 mg, 2.52 mmol, 78%) as a brown solid. m/z 318.1/320.1 (M+H)⁺ (ES+).

Step 7: 6-(Ethylamino)-4-(1-methyl-4-(4-methyl-4H-1,2,4-triazol-3-yl)-1H-pyrazol-5-yl)picolinic acid (AAC-3). Into an 8 mL sealed tube were added the product from step 6 above (ACQ-2) (30 mg, 1 Eq, 94 μmol), oxalic acid (13 mg, 1.5 Eq, 141 μmol), DIPEA (18.3 mg, 1.5 Eq, 140 μmol) and acetic anhydride (14 mg, 1.5 Eq, 141 μmol) in DMF (2 mL) at rt under nitrogen atmosphere. To the above mixture were added Pd(OAc)₂ (2 mg, 0.1 Eq, 10 μmol) and XantPhos (11 mg, 0.2 Eq, 20 μmol) at rt under nitrogen atmosphere. The resulting mixture was stirred for overnight at 100° C. under nitrogen atmosphere then the mixture allowed to cool to rt and concentrated in vacuo. The crude product was purified by Prep-HPLC with the following conditions (Column: Sunfire prep C18 column, 30*150 mm, 5 μm; Mobile Phase A: Water (0.1% FA), Mobile Phase B: MeCN; Flow rate: 60 mL/min; Gradient: 2% B to 20% B in 9 min; Wavelength: 254/220 nm; RT: 6.32) to afford the title compound (AAC-3) (5.2 mg, 16 μmol, 17%) as a white solid. m/z 328.1 (M+H)⁺ (ES+). ¹H NMR (400 MHz, MeOH-d4) δ 8.50 (s, 1H), 7.92 (s, 1H), 7.26 (d, J=1.4 Hz, 1H), 6.78 (s, 1H), 4.00 (s, 3H), 3.55 (s, 3H), 3.34 (s, 1H), 3.32 (s, 1H), 1.23 (t, J=7.2 Hz, 3H).

### Synthesis of 3-(1-Methyl-4-(4-methyl-4H-1,2,4-triazol-3-yl)-1H-pyrazol-5-yl)aniline (Intermediate AAU-1)

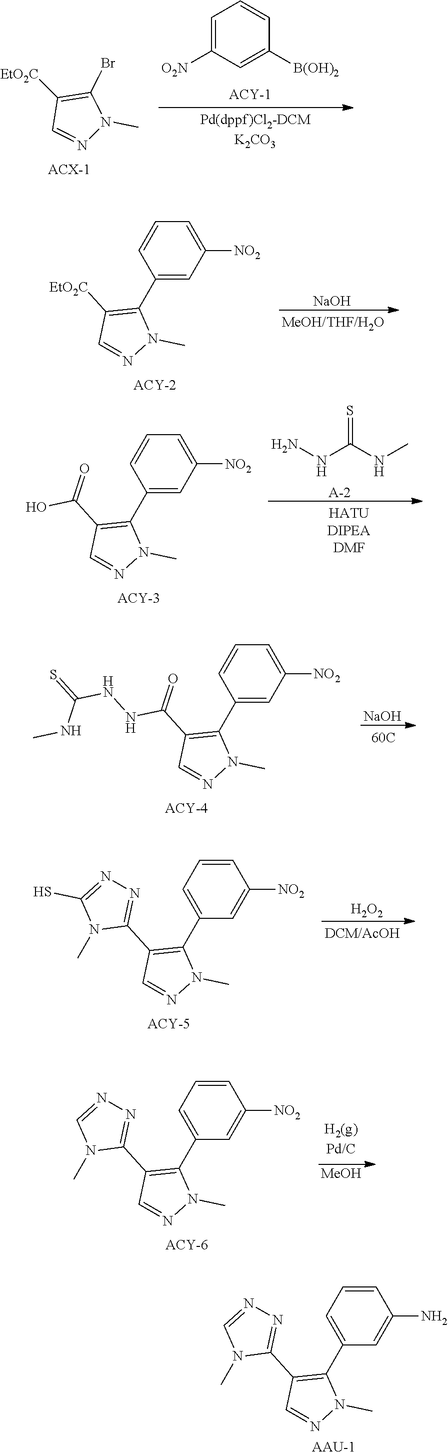

Step 1: Ethyl 1-methyl-5-(3-nitrophenyl)pyrazole-4-carboxylate (ACY-2). To a stirred mixture of ethyl 5-bromo-1-methylpyrazole-4-carboxylate (ACX-1) (1.00 g, 1 Eq, 4.29 mmol), 3-nitrophenylboronic acid (ACY-1) (1.07 g, 1.5 Eq, 6.44 mmol) and sodium carbonate (1.36 g, 3 Eq, 12.9 mmol) in 1,2-dimethoxy-ethane (15 mL) and water (1.5 mL) was added Pd(dppf)Cl₂·DCM (700.7 mg, 0.2 Eq, 858 μmol) at rt under nitrogen atmosphere. The resulting mixture was stirred overnight at 90° C. under nitrogen atmosphere. The mixture was allowed to cool to rt and concentrated in vacuo. The residue was purified by silica gel column chromatography, eluted with petroleum ether/EtOAc (1/1) to afford the sub-title compound (ACY-2) (900 mg, 3.27 mmol, 76%) as a brown solid. m/z 275.3 (M+H)⁺ (ES+).

Step 2: 1-Methyl-5-(3-nitrophenyl)pyrazole-4-carboxylic acid (ACY-3). To a stirred mixture of the product from step 1 above (ACY-2) (500 mg, 1 Eq, 1.82 mmol) in MeOH (8 mL) and THF (8 mL) was added a solution of NaOH (363 mg, 5 Eq, 9.08 mmol) in water (4 mL) at rt. The resulting mixture was stirred for 1.5 h at 60° C. The mixture was allowed to cool to rt, diluted with water, acidified to pH 5 with HCl (aq. 1M) at 0° C. The resulting mixture was diluted with water and extracted with EtOAc (3×80 mL). The combined organic layers were washed with brine (2×80 mL), dried (Na₂SO₄), filtered and concentrated in vacuo to afford the sub-title compound (ACY-3) (440 mg, 1.78 mmol, 98%) as a white solid. m/z 248.2 (M+H)⁺ (ES+).

Step 3: 1-Methyl-N-[(methylcarbamothioyl)amino]-5-(3-nitrophenyl) pyrazole-4-carboxamide (ACY-4). To a stirred mixture of the product from step 2 above (ACY-3) (360 mg, 1 Eq, 1.46 mmol) in DMF (5 mL) were added HATU (831 mg, 1.5 Eq, 2.18 mmol) and DIPEA (565 mg, 3 Eq, 4.37 mmol) at 0° C. The resulting mixture was stirred for 0.5 h at rt. To the above mixture was added 4-methyl-3-thiosemicarbazide (A-2) (161 mg, 1.05 Eq, 1.53 mmol) at rt. The resulting mixture was stirred for additional overnight at rt. The reaction was concentrated affording the sub-title compound (ACY-4) which was used directly in the next step. m/z 335.4 (M+H)⁺ (ES+).

Step 4: 4-Methyl-5-[1-methyl-5-(3-nitrophenyl) pyrazol-4-yl]-1,2,4-triazole-3-thiol (ACY-5). A solution of the product from step 3 above (ACY-4) (486 mg, 1 Eq, 1.45 mmol) in NaOH (aq., 1M) (10 mL) was stirred for 3 h at 60° C. The mixture was allowed to cool to rt, diluted with water, acidified to pH 5 with HCl (aq., 1M) and extracted with EtOAc (3×50 mL). The combined organic layers were washed with brine (2×50 mL), dried (Na₂SO₄), filtered and concentrated in vacuo. The crude product was purified by reverse flash column chromatography with the following conditions: Column, C18; mobile phase, Water (0.1% NH₄HCO₃) and ACN (10% ACN up to 80% in 20 min); Detector, UV 254/220 nm to afford the sub-title compound (ACY-5) (160 mg, 5.6 μmol, 35%) as a brown solid. m/z 317.3 (M+H)⁺ (ES+).

Step 5: 4-Methyl-3-[1-methyl-5-(3-nitrophenyl)pyrazol-4-yl]-1,2,4-triazole (ACY-6). To a stirred solution of the product from step 4 above (ACY-5) (160 mg, 1 Eq, 506 μmol) in DCM (20 mL) was added acetic acid (30 mg, 1 Eq, 506 μmol) and hydrogen peroxide (344 mg, 30% Wt, 6 Eq, 3.04 mmol) at 0° C. The resulting mixture was stirred for 3 h at rt then concentrated in vacuo and purified by silica gel column chromatography, eluted with DCM/MeOH (10/1) to afford the sub-title compound (ACY-6) (120 mg, 422 μmol, 83%) as a brown oil. m/z 285.3 (M+H)⁺ (ES+).

Step 6: 3-[2-Methyl-4-(4-methyl-1,2,4-triazol-3-yl) pyrazol-3-yl]aniline (AAU-1). To a stirred solution of the product from step 5 above (ACY-6) (120 mg, 1 Eq, 422 μmol) in MeOH (13 mL) was added Pd/C 39 (44.9 mg, 10% Wt, 0.1 Eq, 42.2 μmol) portion-wise at rt under nitrogen atmosphere. The mixture was hydrogenated at rt for 2 h under hydrogen atmosphere using a hydrogen balloon. The mixture was filtered through a Celite pad and concentrated in vacuo. The filtrate was concentrated to afford the sub-title compound (AAU-1) (60 mg, 236 μmol, 56%) as a white solid and used in the next step without further purification. m/z 255.3 (M+H)⁺ (ES+).

### Example 1: Synthesis of (2-(2′-(4-Methyl-4H-1,2,4-triazol-3-yl)-[1,1′-biphenyl]-3-yl)-7-(trifluoromethyl)benzo[d]oxazol-5-yl)methanol (F-5)

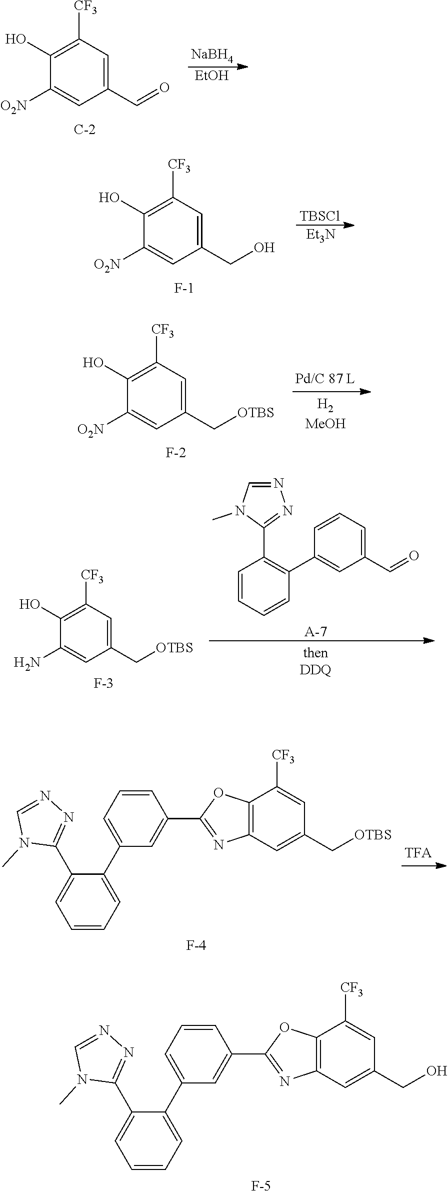

Step 1: 4-(Hydroxymethyl)-2-nitro-6-(trifluoromethyl)phenol (F-1). To a stirring solution of intermediate (C-2) (573 mg, 90% Wt, 1 Eq, 2.19 mmol) in EtOH (15 mL) at rt, was added NaBH₄ (124 mg, 1.5 Eq, 3.29 mmol) portion wise and the reaction stirred at rt for 24 h. The reaction mixture was quenched with water and the solvent was removed in vacuo. The crude product was purified by chromatography on silica gel (40 g cartridge, 5-50% MeOH/DCM) to afford the sub-title compound (F-1) (326 mg, 1.4 mmol, 62%, 99% Purity) as a dark orange oil. m/z 236.1 (M−H)⁻ (ES−). ¹H NMR (400 MHz, DMSO-d6) δ 11.18 (s, 1H), 8.19 (d, J=2.1 Hz, 1H), 7.93 (d, J=2.2 Hz, 1H), 5.46 (s, 1H), 4.53 (s, 2H).

Step 2: 4-(((tert-Butyldimethylsilyl)oxy)methyl)-2-nitro-6-(trifluoromethyl)phenol (F-2). To a solution of the product from step 1 above (F-1) (130 mg, 1 Eq, 548 μmol) and tert-butylchlorodimethylsilane (99.2 mg, 1.2 Eq, 658 μmol) in DMF (5 mL) was added triethylamine (66.6 mg, 91.7 μL, 1.2 Eq, 658 μmol). The reaction mixture was stirred at rt for 3 days. The reaction mixture was diluted with EtOAc (10 mL) and transferred into a separating funnel. The layer was washed with sat. aq. sol. of NH₄Cl (2×5 mL) and brine. The combined organic layers were collected, dried (MgSO₄), filtered and concentrated in vacuo. The crude product was purified by chromatography on silica gel (24 g cartridge, 0-10% EtOAc/isohexane) to afford the sub-title compound (F-2) (150 mg, 0.42 mmol, 77%, 99% Purity) as alight yellow solid. m/z 350.1 (M−H)⁻ (ES−). ¹H NMR (400 MHz, DMSO-d6) δ 11.23 (s, 1H), 8.18 (d, J=2.1 Hz, 1H), 7.91 (d, J=2.2 Hz, 1H), 4.76 (s, 2H), 0.91 (s, 9H), 0.09 (s, 6H).

Step 3: 2-Amino-4-(((tert-butyldimethylsilyl)oxy)methyl)-6-(trifluoromethyl)phenol (F-3). To a solution of the product from step 2 above (F-2) (140 mg, 1 Eq, 398 μmol) in MeOH (2 mL) was added Pd/C 87 L (14 mg, 0.33 Eq, 0.13 mmol). The reaction mixture was stirred at 45° C. with 5 atm of H₂ for 16 h. The catalyst was filtered, and the solvent was removed in vacuo to afford the sub-title compound (F-3) (43 mg, 0.13 mmol, 32%, 95% Purity) as a brown solid. m/z 322.4 (M+H)⁺ (ES+). ¹H NMR (400 MHz, DMSO-d6) δ 6.85-6.79 (m, 1H), 6.67 (d, J=2.0 Hz, 1H), 4.54 (s, 2H), 3.17 (s, 1H), 0.89 (s, 8H), 0.06 (s, 6H). Three exchangeable protons not observed.

Step 4: 5-(((tert-Butyldimethylsilyl)oxy)methyl)-2-(2′-(4-methyl-4H-1,2,4-triazol-3-yl)-[1,1′-biphenyl]-3-yl)-7-(trifluoromethyl)benzo[d]oxazole (F-4). To a solution of intermediate (A-7) (41 mg, 80% Wt, 1 Eq, 0.12 mmol) in MeOH (2 mL) was added the product from step 3 above (F-3) (40 mg, 1 Eq, 0.12 mmol). The resulting mixture was stirred at 45° C. for 12 h. After concentration in vacuo, the residue was dissolved in DCM (2 mL) and 4,5-dichloro-3,6-dioxocyclohexa-1,4-diene-1,2-dicarbonitrile (31 mg, 1.1 Eq, 0.14 mmol) was then added. After stirring at rt for 1 h, the resulting mixture was diluted with DCM (5 mL) and washed sequentially with sat. aq. sol. of NaHCO₃ (2×5 mL) and brine (5 mL). The organic layer was dried (Na₂SO₄) and concentrated in vacuo. The crude product was purified by chromatography on silica gel (12 g cartridge, 0-10% MeOH/DCM) to afford the sub-title compound (F-4) (44 mg, 76 μmol, 61%, 98% Purity) as a tan solid. m/z 564.7 (M+H)⁺ (ES+). ¹H NMR (400 MHz, DMSO-d6) δ 8.39 (s, 1H), 8.14 (dt, J=7.9, 1.4 Hz, 1H), 8.03 (s, 1H), 7.96 (t, J=1.8 Hz, 1H), 7.79-7.69 (m, 3H), 7.69-7.61 (m, 2H), 7.59 (t, J=7.8 Hz, 1H), 7.39 (dt, J=7.8, 1.5 Hz, 1H), 4.92 (s, 2H), 3.14 (s, 3H), 0.93 (s, 9H), 0.12 (s, 6H).

Step 5: (2-(2′-(4-Methyl-4H-1,2,4-triazol-3-yl)-[1,1′-biphenyl]-3-yl)-7-(trifluoromethyl)benzo[d]oxazol-5-yl)methanol (F-5). To a solution of the product from step 4 above (F-4) (43.0 mg, 90% Wt, 1 Eq, 68.5 μmol) in THF (2 mL) was added TBAF (19.7 mg, 75.4 μL, 1 molar, 1.1 Eq, 75.4 μmol) and the mixture was stirred at rt for 2 h. The reaction mixture was concentrated under reduced pressure. The crude product was purified by chromatography on RP Flash C18 (12 g cartridge, 15-75% (0.1% formic acid in MeCN)/(0.1% formic acid in water)). The product fractions were combined diluted with EtOAc (10 mL) and washed with sat. aq. sol. of NH₄Cl (2×5 mL), sat. aq. sol. of NaHCO₃ (5 mL) and brine (5 mL). The combined organic layers were dried (MgSO₄), filtered and concentrated in vacuo to afford the title compound (F-5) (20 mg, 44 μmol, 64%, 99% Purity) as a tan solid. m/z 451.3 (M+H)⁺ (ES+). ¹H NMR (400 MHz, Methanol-d4) δ 8.40 (s, 1H), 8.22 (dt, J=7.8, 1.4 Hz, 1H), 8.10 (t, J=1.8 Hz, 1H), 8.01-7.97 (m, 1H), 7.85-7.75 (m, 2H), 7.75-7.71 (m, 1H), 7.70-7.67 (m, 2H), 7.59 (t, J=7.8 Hz, 1H), 7.48 (ddd, J=7.8, 1.9, 1.2 Hz, 1H), 4.81 (s, 2H), 3.25 (s, 3H). One exchangeable proton not observed.

### Example 2: Synthesis of Methyl 2-(2′-(4-methyl-4H-1,2,4-triazol-3-yl)-[1,1′-biphenyl]-3-yl)-7-(trifluoromethyl)benzo[d]oxazole-5-carboxylate (G-1)

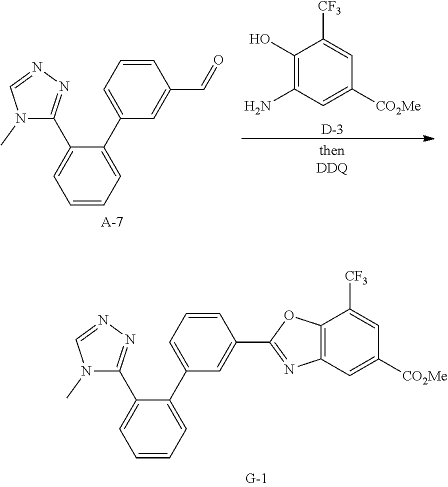

To a solution of intermediate (A-7) (120 mg, 95% Wt, 1 Eq, 433 μmol) in MeOH (2 mL) was added intermediate (D-3) (102 mg, 1 Eq, 433 μmol) and stirred at 45° C. for 12 h. The reaction mixture was concentrated in vacuo, the residue dissolved in DCM (2 mL) and 4,5-dichloro-3,6-dioxocyclohexa-1,4-diene-1,2-dicarbonitrile (108 mg, 1.1 Eq, 476 μmol) was added and stirred at rt for 1 h. The mixture was diluted DCM (5 mL), washed with sat. aq. sol. of NaHCO₃ (2×5 mL) and brine (5 mL). The organic layer was dried (Na₂SO₄) and concentrated in vacuo. The crude product was purified by chromatography on silica gel (12 g cartridge, 0-10% MeOH/DCM) followed by chromatography on RP Flash C18 (12 g cartridge, 15-75% (0.1% formic acid in MeCN)/(0.1% formic acid in water)) to afford the title compound (G-1) (71 mg, 0.15 mmol, 34%, 99% Purity) as a pale white solid. m/z 479.1 (M+H)⁺ (ES+). ¹H NMR (400 MHz, DMSO-d6) δ 8.64-8.60 (m, 1H), 8.40 (s, 1H), 8.26 (dd, J=1.6, 0.8 Hz, 1H), 8.16 (dt, J=7.9, 1.3 Hz, 1H), 7.97 (t, J=1.8 Hz, 1H), 7.80-7.69 (m, 2H), 7.68-7.57 (m, 3H), 7.43 (dt, J=7.9, 1.4 Hz, 1H), 3.95 (s, 3H), 3.16 (s, 3H).

### Example 3: Synthesis of 2-(2′-(4-Methyl-4H-1,2,4-triazol-3-yl)-[1,1′-biphenyl]-3-yl)-7-(trifluoromethyl)benzo[d]oxazole-5-carboxylic Acid (H-1)

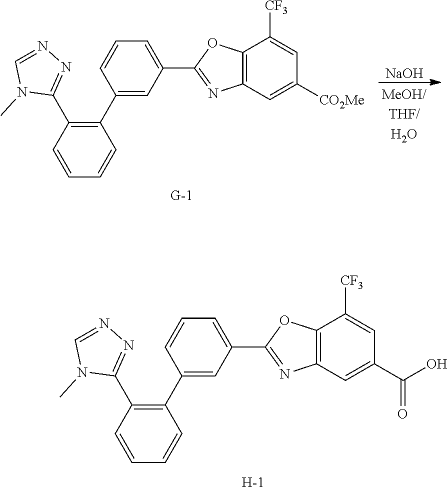

To a solution of intermediate (G-1) (30 mg, 1 Eq, 63 μmol) in MeOH/THF/Water (1:1:1, 3 mL) was added NaOH (3.0 mg, 75 μL, 1 molar, 1.2 Eq, 75 μmol) and stirred at rt for 5 days. The reaction mixture was diluted with water (10 mL) and extracted with EtOAc (5 mL). The aqueous layer was acidified with aq. HCl 1M and extracted with DCM (3×5 mL). The combined organic extracts were washed with brine (5 mL), dried (MgSO₄) and concentrated in vacuo to afford the title compound (H-1) (25 mg, 53 μmol, 85%, 99% Purity) as a white solid. m/z 465.2 (M+H)⁺ (ES+). ¹H NMR (400 MHz, DMSO-d6) δ 13.64 (s, 1H), 8.59 (d, J=1.5 Hz, 1H), 8.40 (s, 1H), 8.25 (s, 1H), 8.17 (dt, J=7.8, 1.4 Hz, 1H), 7.98 (t, J=1.8 Hz, 1H), 7.80-7.70 (m, 2H), 7.68-7.57 (m, 3H), 7.42 (dt, J=7.9, 1.4 Hz, 1H), 3.16 (s, 3H).

### Example 4: Synthesis of (S)-(2-(2′-(4-Methyl-4H-1,2,4-triazol-3-yl)-[1,1′-biphenyl]-3-yl)-7-(trifluoromethyl)benzo[d]oxazol-5-yl)(3-methylpiperidin-1-yl)methanone (I-2)

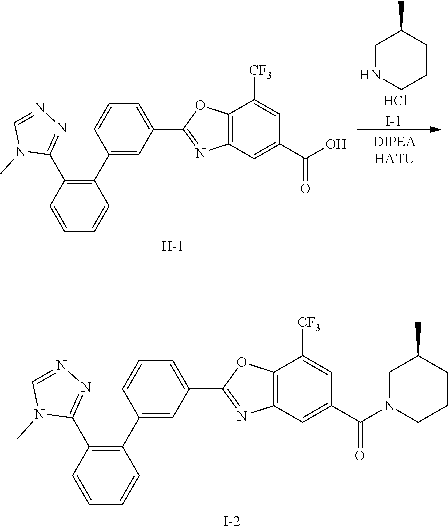

To a solution of intermediate (H-1) (20 mg, 1 Eq, 43 μmol) in DMF (1 mL) was added HATU (20 mg, 1.2 Eq, 52 μmol) and DIPEA (22 mg, 30 μL, 4 Eq, 0.17 mmol). The reaction mixture was stirred for 20 min at rt then (S)-3-methylpiperidine, HCl (I-1) (7.0 mg, 1.2 Eq, 52 μmol) was added and stirred at rt for 3 h. The solvent was removed in vacuo. The residue was diluted with EtOAc (25 mL) and washed with sat. aq. sol. of NH₄Cl (2×5 mL), sat. aq. sol. of NaHCO₃ (2×5 mL) and brine (5 mL). The combined organic layers were dried (MgSO₄), filtered and concentrated in vacuo. The crude product was purified by chromatography on silica gel (12 g cartridge, 0-20% MeOH/DCM) to afford the title compound (I-2) (17 mg, 31 μmol, 71%, 98% Purity) as a pale yellow solid. m/z 546.3 (M+H)⁺ (ES+). ¹H NMR (400 MHz, DMSO-d6) δ 8.30 (s, 1H), 8.15 (dt, J=7.8, 1.4 Hz, 1H), 8.09 (d, J=1.4 Hz, 1H), 7.99 (t, J=1.8 Hz, 1H), 7.79-7.68 (m, 3H), 7.68-7.56 (m, 3H), 7.46 (dt, J=7.9, 1.4 Hz, 1H), 3.89 (s, 2H), 3.19 (s, 3H), 2.80-2.62 (m, 1H), 1.91-1.78 (m, 1H), 1.69 (ddt, J=13.5, 7.3, 3.6 Hz, 2H), 1.59-1.42 (m, 1H), 1.32-1.25 (m, 1H), 1.24-1.13 (m, 1H), 0.87 (d, J=6.6 Hz, 3H).

### Example 5: Synthesis of 2-(2-(2′-(4-Methyl-4H-1,2,4-triazol-3-yl)-[1,1′-biphenyl]-3-yl)-7-(trifluoromethyl)benzo[d]oxazol-5-yl)propan-2-ol (J-1)

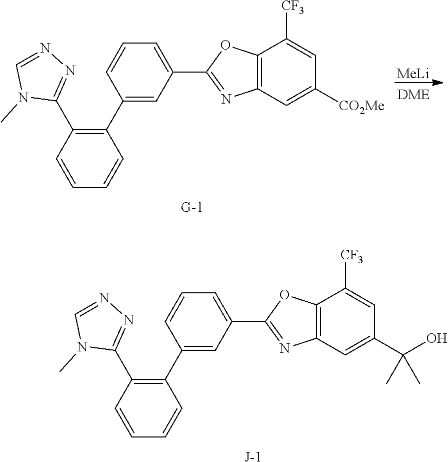 [SMILES: "MeLi" ; COC(=O)c1cc(C(F)(F)F)c2oc(-c3cccc(-c4ccccc4-c4nncn4C)c3)nc2c1 ; Cn1cnnc1-c1ccccc1-c1cccc(-c2nc3cc(C(C)(C)O)cc(C(F)(F)F)c3o2)c1 ; "J-1"]

To a solution of intermediate (G-1) (18.0 mg, 1 Eq, 37.6 μmol) in DME (1 mL) was added methyllithium (2.48 mg, 70.5 μL, 1.6 molar, 3.0 Eq, 113 μmol) drop-wise at 0° C. The mixture was stirred for 1 h at this temperature, then slowly warmed to rt and stirred 72 h. The mixture was quenched with slow addition of a sat. aq. sol. of NH₄Cl. The reaction mixture was diluted with EtOAc (10 mL), washed with sat. aq. sol. of NH₄Cl (2×5 mL), sat. aq. sol. of NaHCO₃ (2×5 mL) and brine (5 mL). The combined organic layers were collected, dried (MgSO₄), filtered and concentrated in vacuo. The crude product was purified by preparative HPLC (Waters, Basic (0.3% ammonia), Basic, Waters X-Bridge BEH C18 ODB, 5 μm, 30×100 mm column 0-100% MeCN in water) to afford the title compound (J-1) (3 mg, 6 μmol, 20%, 99% Purity) as a white solid. m/z 479.1 (M+H)⁺ (ES+). ¹H NMR (400 MHz, Methanol-d4) δ 8.40 (s, 1H), 8.24 (dt, J=7.8, 1.4 Hz, 1H), 8.12 (dt, J=6.5, 1.5 Hz, 2H), 7.92-7.89 (m, 1H), 7.86-7.77 (m, 2H), 7.71-7.68 (m, 2H), 7.61 (t, J=7.8 Hz, 1H), 7.53-7.46 (m, 1H), 3.26 (s, 3H), 1.66 (s, 6H). One exchangeable proton not observed.

### Example 6: Synthesis of 2-(2′-(4-Methyl-4H-1,2,4-triazol-3-yl)-[1,1′-biphenyl]-3-yl)-7-(trifluoromethyl)benzo[d]oxazole (K-2)

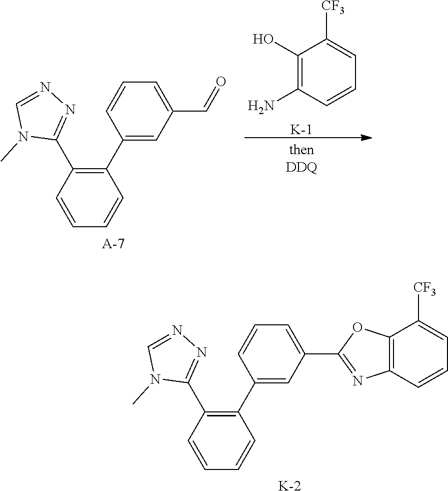

To a solution of intermediate (A-7) (30 mg, 80% Wt, 1 Eq, 91 μmol) in MeOH (1 mL) was added 2-amino-6-(trifluoromethyl)phenol (K-1) (16 mg, 1 Eq, 91 μmol). The resulting mixture was stirred at 45° C. for 12 h. After concentration under reduced pressure, the residue was dissolved in DCM (2 mL) and 4,5-dichloro-3,6-dioxocyclohexa-1,4-diene-1,2-dicarbonitrile (23 mg, 1.1 Eq, 0.10 mmol) was then added. After stirring at rt for 1 h, the resulting mixture was diluted with additional DCM (5 mL) and washed sequentially with sat. sol. of NaHCO₃ (2×5 mL) and brine (5 mL). The organic extract was separated, dried (Na₂SO₄) and concentrated in vacuo. The crude product was purified by chromatography on RP Flash C18 (4 g cartridge, 15-75% MeCN/10 mM ammonium bicarbonate) to afford the title compound (K-2) (24 mg, 57 μmol, 62%, 99% Purity) as a dark brown solid. m/z 421.2 (M+H)⁺ (ES+). ¹H NMR (400 MHz, Methanol-d4) δ 8.40 (s, 1H), 8.24 (dt, J=7.8, 1.4 Hz, 1H), 8.12 (t, J=1.8 Hz, 1H), 8.03 (d, J=8.0 Hz, 1H), 7.84-7.77 (m, 2H), 7.74 (dt, J=7.7, 0.9 Hz, 1H), 7.71-7.67 (m, 2H), 7.64-7.56 (m, 2H), 7.49 (dt, J=7.9, 1.4 Hz, 1H), 3.26 (s, 3H).

### Example 7: Synthesis of (R)-2-(2′-(4-Methyl-4H-1,2,4-triazol-3-yl)-[1,1′-biphenyl]-3-yl)-5-((3-methylpiperidin-1-yl)methyl)-7-(trifluoromethyl)benzo[d]oxazole (L-4)

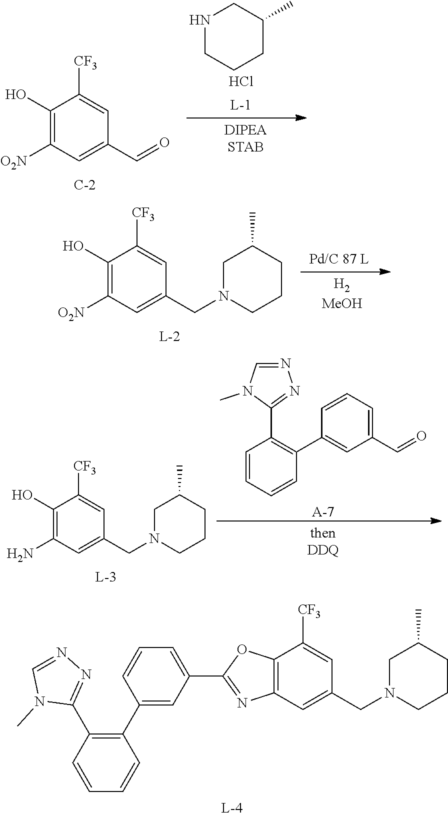

Step 1: (R)-4-((3-Methylpiperidin-1-yl)methyl)-2-nitro-6-(trifluoromethyl)phenol (L-2). NaBH(OAc)₃ (161 mg, 6.0 Eq, 758 μmol) was added to a stirred solution of intermediate (C-2) (30.0 mg, 99% Wt, 1 Eq, 126 μmol), (R)-3-methylpiperidine, HCl (L-1) (34.3 mg, 2.0 Eq, 253 μmol) and DIPEA (49.0 mg, 65.1 μL, 3.0 Eq, 379 μmol) in THF (1.3 mL) and stirred at rt overnight. The reaction mixture was concentrated in vacuo. The crude product was purified by chromatography on RP Flash C18 (4 g cartridge, 5-50% (0.7 M Ammonia/MeOH)/DCM) followed by purification on a column of SCX (500 mg) in MeOH. The column was washed with MeOH and then the product was eluted with 0.7 M ammonia in MeOH. The resultant mixture was concentrated in vacuo to afford the sub-title compound (L-2) (34 mg, 0.11 mmol, 84%, 99% Purity) as a light yellow solid. m/z 319.5 (M+H)⁺ (ES+). ¹H NMR (400 MHz, DMSO-d6) δ 8.97 (s, 1H), 7.94 (s, 1H), 7.44 (s, 1H), 4.00 (s, 2H), 3.24-3.17 (m, 2H), 2.66 (d, J=4.9 Hz, 1H), 1.90-1.65 (m, 4H), 1.60 (s, 1H), 1.03 (s, 1H), 0.88 (d, J=6.5 Hz, 3H).

Step 2: (R)-2-Amino-4-((3-methylpiperidin-1-yl)methyl)-6-(trifluoromethyl)phenol (L-3). To a solution of the product from step 1 above (L-2) (35 mg, 1 Eq, 0.11 mmol) in THF (1 mL) and EtOAc (1 mL) was added Pd/C type 87L (3.5 mg, 0.30 Eq, 33 μmol). The reaction mixture was stirred at 45° C. with 5 atm of H₂ for 16 h. The catalyst was removed by filtration, and the solvent removed in vacuo to afford the sub-title compound (L-3) (20 mg, 55 μmol, 50%, 80% Purity) as a red oil. m/z 289.2 (M+H)⁺ (ES+). ¹H NMR (400 MHz, Methanol-d4) δ 6.91 (d, J=2.1 Hz, 1H), 6.85 (d, J=2.1 Hz, 1H), 3.51 (s, 2H), 2.94 (dd, J=20.2, 10.4 Hz, 2H), 2.06 (td, J=11.9, 3.1 Hz, 1H), 1.82-1.59 (m, 5H), 1.02-0.92 (m, 1H), 0.90 (d, J=6.0 Hz, 3H). 3 Exchangeable protons not observed.

Step 3: (R)-2-(2′-(4-Methyl-4H-1,2,4-triazol-3-yl)-[1,1′-biphenyl]-3-yl)-5-((3-methylpiperidin-1-yl)methyl)-7-(trifluoromethyl)benzo[d]oxazole (L-4). To a solution of intermediate (A-7) (18 mg, 80% Wt, 1 Eq, 55 μmol) in MeOH (1 mL) was added the product from step 2 above (L-3) (20 mg, 80% Wt, 1 Eq, 55 μmol). The resulting mixture was stirred at 45° C. for 12 h. After concentration in vacuo, the residue was dissolved in DCM (2 mL) and 4,5-dichloro-3,6-dioxocyclohexa-1,4-diene-1,2-dicarbonitrile (14 mg, 1.1 Eq, 61 μmol) was then added. After stirring at rt for 1 h, the resulting mixture was diluted with additional DCM (5 mL) and washed sequentially with sat. aq. sol. of NaHCO₃ (2×5 mL) and brine (5 mL). The organics were extracted, dried (Na₂SO₄) and concentrated in vacuo. The crude was purified by preparative HPLC (Waters, Basic (0.3% ammonia), Waters XBridge BEH C18 ODB, 5 μm, 30×100 mm column, 0.100% MeCN in water) to afford the title compound (L-4) (8 mg, 0.01 mmol, 30%, 98% Purity) as a white solid. m/z 532.4 (M+H)⁺ (ES+). ¹H NMR (400 MHz, Methanol-d4) δ 8.40 (s, 1H), 8.26-8.20 (m, 1H), 8.11 (s, 1H), 7.99 (s, 1H), 7.86-7.76 (m, 2H), 7.74 (s, 1H), 7.71-7.67 (m, 2H), 7.61 (t, J=7.8 Hz, 1H), 7.49 (dt, J=7.9, 1.4 Hz, 1H), 3.71 (s, 2H), 3.26 (s, 3H), 2.87 (dd, J=14.4, 9.4 Hz, 2H), 2.02 (td, J=11.4, 3.1 Hz, 1H), 1.84-1.56 (m, 5H), 1.04-0.86 (m, 4H).

### Example 8: Synthesis of tert-Butyl ((1R,2R)-2-hydroxycyclopentyl)((2-(2′-(4-methyl-4H-1,2,4-triazol-3-yl)-[1,1′-biphenyl]-3-yl)-7-(trifluoromethyl)benzo[d]oxazol-5-yl)methyl)carbamate (M-5)

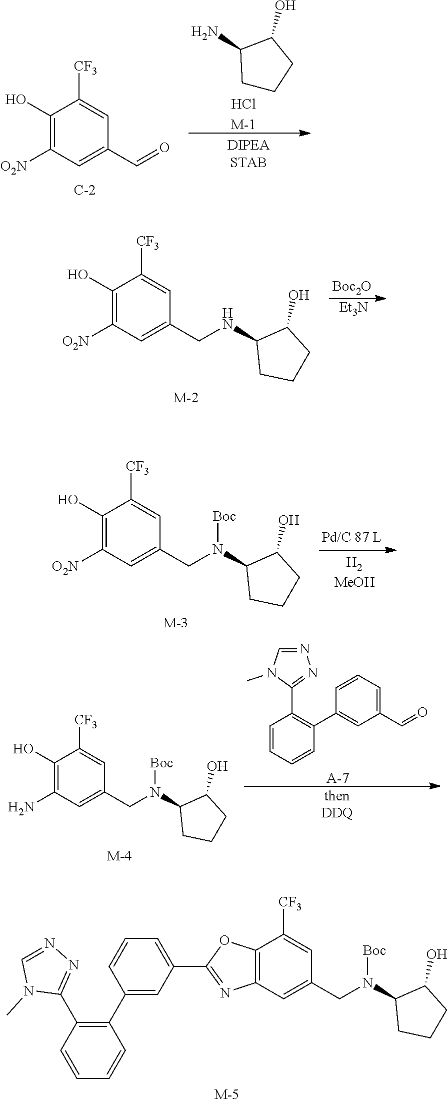

Step 1: 4-((((1R,2R)-2-Hydroxycyclopentyl)amino)methyl)-2-nitro-6-(trifluoromethyl)phenol (M-2). NaBH(OAc)₃ (161 mg, 6.0 Eq, 758 μmol) was added to a stirred solution of intermediate (C-2) (30.0 mg, 99% Wt, 1 Eq, 126 μmol), (1R,2R)-2-aminocyclopentan-1-ol, HCl (M-1) (34.8 mg, 2.0 Eq, 253 μmol) and DIPEA (49.0 mg, 65.1 μL, 3.0 Eq, 379 μmol) in THF (1.3 mL) and stirred at rt overnight. The reaction mixture was concentrated in vacuo and the crude product was purified by chromatography on RP Flash C18 (4 g cartridge, 5-50% MeCN/10 mM ammonium bicarbonate) followed by further purification using a column of SCX (500 mg) in MeOH. The column was washed with MeOH and then the product was eluted with 0.7 M ammonia in MeOH. The resultant mixture was concentrated in vacuo to afford the sub-title compound (M-2) (36 mg, 0.11 mmol, 85%, 95% Purity) as a light yellow solid. m/z 321.2 (M+H)⁺ (ES+). ¹H NMR (400 MHz, Methanol-d4) δ 7.94 (d, J=2.6 Hz, 1H), 7.56 (d, J=2.6 Hz, 1H), 4.41 (q, J=6.6 Hz, 1H), 4.14 (d, J=13.4 Hz, 1H), 4.02 (d, J=13.3 Hz, 1H), 3.37 (d, J=7.1 Hz, 1H), 2.31 (qd, J=8.1, 7.3, 3.8 Hz, 1H), 2.15 (dq, J=13.7, 6.7 Hz, 1H), 1.85 (hd, J=9.0, 1.6 Hz, 3H), 1.71 (dq, J=14.1, 7.0 Hz, 1H). Three exchangeable protons not observed.

Step 2: tert-Butyl (4-hydroxy-3-nitro-5-(trifluoromethyl)benzyl)((1R,2R)-2-hydroxycyclopentyl)carbamate (M-3). To a solution of the product from step 1 above (M-2) (36 mg, 1 Eq, 0.11 mmol) and di-tert-butyl dicarbonate (27 mg, 1.1 Eq, 0.12 mmol) in DCM (2 mL) and THF (2 mL) was added NEt₃ (23 mg, 31 μL, 2 Eq, 0.22 mmol) and the reaction mixture was stirred at rt for 16 h. The reaction mixture was concentrated in vacuo. The crude product was purified by chromatography on silica gel (4 g cartridge, 0-100% EtOAc/isohexane) to afford the sub-title compound (M-3) (35 mg, 75 μmol, 67%, 90% Purity) as a pale yellow oil. m/z 419.0 (M−H)⁻ (ES−). ¹H NMR (400 MHz, Chloroform-d) δ 11.11 (s, 1H), 8.23 (d, J=2.2 Hz, 1H), 7.85 (s, 1H), 4.45 (h, J=16.9 Hz, 2H), 4.21-3.91 (m, 2H), 1.97 (dd, J=13.0, 6.7 Hz, 2H), 1.92-1.80 (m, 2H), 1.80-1.60 (m, 2H), 1.45 (s, 9H). One exchangeable proton not observed.

Step 3: tert-Butyl (3-amino-4-hydroxy-5-(trifluoromethyl)benzyl)((1R,2R)-2-hydroxycyclopentyl)carbamate (M-4). To a solution of the product from step 2 above (M-3) (35 mg, 90% Wt, 1 Eq, 75 μmol) in MeOH (1.5 mL) was added Pd/C type 87L (3.5 mg, 0.44 Eq, 33 μmol). The reaction mixture was stirred at 45° C. with 5 atm of H₂ for 16 h. The catalyst was removed by filtration and the solvent was removed in vacuo to afford the sub-title compound (M-4) (24 mg, 55 μmol, 74%, 90% Purity) as a red oil. m/z 389.2 (M−H)⁻ (ES−). ¹H NMR (400 MHz, Methanol-d4) δ 6.85 (d, J=2.1 Hz, 1H), 6.76 (d, J=2.1 Hz, 1H), 4.42 (s, 1H), 4.29-4.15 (m, 2H), 4.03-3.78 (m, 1H), 1.91 (dt, J=13.0, 6.7 Hz, 1H), 1.78 (d, J=12.7 Hz, 1H), 1.63 (td, J=6.3, 5.8, 3.1 Hz, 4H), 1.55-1.30 (m, 9H). Four exchangeable protons not observed.

Step 4: tert-Butyl ((1R,2R)-2-hydroxycyclopentyl)((2-(2′-(4-methyl-4H-1,2,4-triazol-3-yl)-[1,1′-biphenyl]-3-yl)-7-(trifluoromethyl)benzo[d]oxazol-5-yl)methyl)carbamate (M-5). To a solution of intermediate (A-7) (17 mg, 80% Wt, 1 Eq, 52 μmol) in MeOH (1 mL) was added the product from step 3 above (M-4) (24 mg, 85% Wt, 1 Eq, 52 μmol). The resulting mixture was stirred at 45° C. for 12 h. After concentration in vacuo, the residue was dissolved in DCM (2 mL) and 4,5-dichloro-3,6-dioxocyclohexa-1,4-diene-1,2-dicarbonitrile (13 mg, 1.1 Eq, 57 μmol) was added. After stirring at rt for 1 h, the resulting mixture was diluted with additional DCM (5 mL) and washed sequentially with sat. aq. sol. of NaHCO₃ (2×5 mL) and brine (5 mL). The organic layer was extracted, dried (Na₂SO₄) and concentrated in vacuo. The crude product was purified by chromatography on RP Flash C18 (4 g cartridge, 15-75% MeCN/10 mM ammonium bicarbonate) to afford the title compound (M-5) (23 mg, 36 μmol, 69%, 98% Purity) as a pale brown solid. m/z 634.0 (M+H)⁺ (ES+). ¹H NMR (400 MHz, Chloroform-d) δ 8.89 (s, 1H), 8.22 (d, J=8.7 Hz, 2H), 7.85 (s, 1H), 7.78-7.65 (m, 3H), 7.62 (d, J=7.0 Hz, 1H), 7.52 (d, J=1.6 Hz, 1H), 7.46 (t, J=7.6 Hz, 1H), 7.30 (d, J=7.5 Hz, 1H), 4.61 (d, J=15.0 Hz, 2H), 4.16 (s, 2H), 3.18 (s, 3H), 2.01-1.77 (m, 2H), 1.77-1.31 (m, 14H). One exchangeable proton not observed.

### Example 9: Synthesis of (1R,2R)-2-(((2-(2′-(4-Methyl-4H-1,2,4-triazol-3-yl)-[1,1′-biphenyl]-3-yl)-7-(trifluoromethyl)benzo[d]oxazol-5-yl)methyl)amino)cyclopentan-1-ol (N-1)

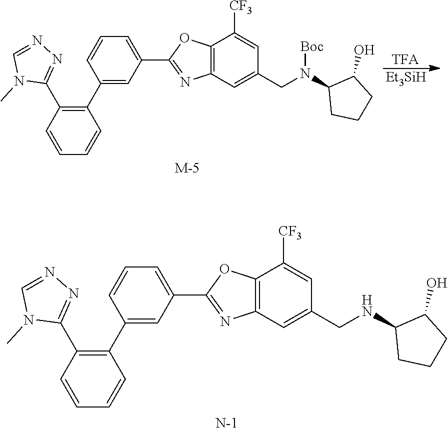

To a solution compound (M-5) (20.0 mg, 1 Eq, 31.6 μmol) in DCM (0.5 mL) were successively added triethylsilane (7.34 mg, 10.1 μL, 2 Eq, 63.1 μmol), water (2.84 mg, 2.84 μL, 5 Eq, 158 μmol) and TFA (162 mg, 109 μL, 45 Eq, 1.42 mmol). The reaction mixture was stirred at rt for 30 min. The mixture was diluted with DCM/water (1:1, 10 mL) and the aqueous phase was extracted using DCM (2×2 mL). The aqueous phase was basified using 2M NaOH solution and extracted using DCM (3×5 mL). The combined organic extracts were washed with brine (5 mL), dried (MgSO₄) and concentrated in vacuo to afford the title compound (N-1) (9 mg, 0.02 mmol, 50%, 99% Purity) as a tan solid. m/z 534.1 (M+H)⁺ (ES+). ¹H NMR (400 MHz, Methanol-d4) δ 8.40 (s, 1H), 8.23 (dt, J=7.9, 1.4 Hz, 1H), 8.11 (t, J=1.8 Hz, 1H), 8.04 (d, J=1.5 Hz, 1H), 7.85-7.76 (m, 3H), 7.71-7.66 (m, 2H), 7.60 (t, J=7.8 Hz, 1H), 7.48 (dt, J=7.9, 1.4 Hz, 1H), 4.05-3.96 (m, 3H), 3.26 (s, 3H), 2.94 (td, J=7.3, 5.0 Hz, 1H), 2.13-1.93 (m, 2H), 1.82-1.68 (m, 2H), 1.59 (ddt, J=12.6, 8.2, 6.0 Hz, 1H), 1.46 (dq, J=12.9, 8.0 Hz, 1H). Two exchangeable protons not observed.

### Example 10: Synthesis of (R)-2-(2′-(4-Methyl-4H-1,2,4-triazol-3-yl)-[1,1′-biphenyl]-3-yl)-5-((2-methylmorpholino)methyl)-7-(trifluoromethyl)benzo[d]oxazole (O-4)

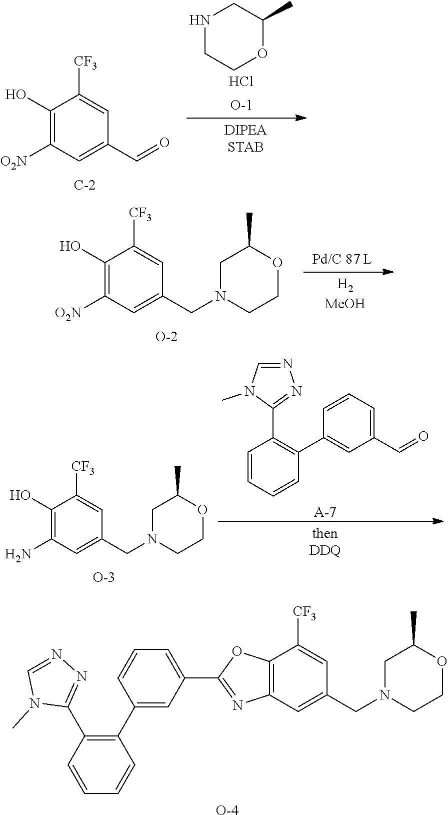

Step 1: (R)-4-((2-Methylmorpholino)methyl)-2-nitro-6-(trifluoromethyl)phenol (0-2). NaBH(OAc)₃ (535.4 mg, 6.0 Eq, 2.526 mmol) was added to a stirred solution of intermediate (C-2) (100.0 mg, 99% Wt, 1 Eq, 421.1 μmol), (R)-2-methylmorpholine, HCl (0-1) (115.9 mg, 2.0 Eq, 842.1 μmol) and DIPEA (163.3 mg, 217 μL, 3.0 Eq, 1.263 mmol) in THF (5 mL) and stirred at rt overnight. The reaction mixture was concentrated in vacuo. The crude product was purified by chromatography on RP Flash C18 (12 g cartridge, 5-50% (0.7 M ammonia/MeOH)/DCM). The product containing fractions were collected and loaded onto a column of SCX (3 g) in MeOH. The column was washed with MeOH (20 mL) and the product was eluted with 0.7 M ammonia in MeOH (20 mL). The resultant mixture was concentrated in vacuo to afford the sub-title compound (0-2) (144 mg, 0.36 mmol, 85%, 80% Purity) as a pale yellow solid. m/z 321.5 (M+H)⁺ (ES+). ¹H NMR (400 MHz, DMSO-d6) δ 7.71 (d, J=2.6 Hz, 1H), 7.27 (d, J=2.6 Hz, 1H), 3.71 (ddd, J=11.2, 3.4, 1.6 Hz, 1H), 3.64-3.52 (m, 1H), 3.51-3.41 (m, 2H), 3.20 (d, J=1.5 Hz, 2H), 2.67 (dt, J=11.1, 2.0 Hz, 1H), 2.60 (dt, J=11.3, 2.0 Hz, 1H), 2.02-1.92 (m, 1H), 1.65 (dd, J=11.2, 9.9 Hz, 1H), 1.01 (d, J=6.3 Hz, 3H).

Step 2: (R)-2-Amino-4-((2-methylmorpholino)methyl)-6-(trifluoromethyl)phenol (0-3). To a solution of the product from step 1 above (0-2) (144 mg, 80% Wt, 1 Eq, 360 μmol) in THF (1 mL) and EtOAc (1 mL) was added Pd/C type 87L (3.5 mg, 0.092 Eq, 33 μmol). The reaction mixture was stirred at 45° C. with 5 atm of H₂ for 16 h. The catalyst was removed by filtration and the solvent was removed in vacuo. The crude product was loaded onto a column of SCX (500 mg) in MeOH. The column was washed with MeOH (20 mL) and the product eluted with 0.7 M ammonia in MeOH. The resultant mixture was concentrated in vacuo to afford the sub-title compound (0-3) (88 mg, 0.24 mmol, 67%, 80% Purity) as a red oil. m/z 290.8 (M+H)⁺ (ES+). ¹H NMR (400 MHz, DMSO-d6) δ 6.84 (d, J=2.0 Hz, 1H), 6.63 (d, J=2.0 Hz, 1H), 3.77-3.61 (m, 1H), 3.52-3.41 (m, 2H), 3.27 (d, J=2.8 Hz, 2H), 2.64 (d, J=11.1 Hz, 1H), 2.56 (dd, J=11.2, 1.8 Hz, 1H), 1.96 (td, J=11.4, 3.3 Hz, 1H), 1.66 (dd, J=11.2, 9.9 Hz, 1H), 1.01 (d, J=6.4 Hz, 3H). Three exchangeable protons not visible.

Step 3: (R)-2-(2′-(4-Methyl-4H-1,2,4-triazol-3-yl)-[1,1′-biphenyl]-3-yl)-5-((2-methylmorpholino)methyl)-7-(trifluoromethyl)benzo[d]oxazole (0-4). To a solution of intermediate (A-7) (23 mg, 80% Wt, 1 Eq, 69 μmol) in MeOH (1 mL) was added the product from step 2 above (0-3) (25 mg, 80% Wt, 1 Eq, 69 μmol). The resulting mixture was stirred at 45° C. for 12 h. After concentration in vacuo, the residue was dissolved in DCM (2 mL) and 4,5-dichloro-3,6-dioxocyclohexa-1,4-diene-1,2-dicarbonitrile (17 mg, 1.1 Eq, 76 μmol) was then added. After stirring at rt for 1 h, the resulting mixture was diluted with DCM (5 mL) and washed sequentially with sat. aq. sol. of NaHCO₃ (2×5 mL) and brine (5 mL). The organic layer was dried (Na₂SO₄) and concentrated under reduced pressure. The crude product was purified by chromatography on RP Flash C18 (4 g cartridge, 15-75% MeCN/10 mM ammonium bicarbonate) to afford the title compound (0-4) (15 mg, 27 μmol, 40%, 97% Purity) as a tan solid. m/z 534.4 (M+H)⁺ (ES+). ¹H NMR (400 MHz, DMSO-d6) δ 8.39 (s, 1H), 8.13 (dt, J=7.9, 1.4 Hz, 1H), 8.05 (s, 1H), 7.93 (t, J=1.8 Hz, 1H), 7.78-7.69 (m, 3H), 7.67-7.62 (m, 2H), 7.59 (t, J=7.8 Hz, 1H), 7.41 (dt, J=7.9, 1.4 Hz, 1H), 3.80-3.62 (m, 3H), 3.52 (td, J=11.2, 2.5 Hz, 2H), 3.15 (s, 3H), 2.69 (dd, J=28.6, 11.3 Hz, 2H), 2.12 (s, 1H), 1.81 (s, 1H), 1.03 (d, J=6.3 Hz, 3H).

### Example 11: Synthesis of (1S,2S)-2-(((2-(2′-(4-Methyl-4H-1,2,4-triazol-3-yl)-[1,1′-biphenyl]-3-yl)-7-(trifluoromethyl)benzo[d]oxazol-5-yl)methyl)amino)cyclopentan-1-ol (P-6)

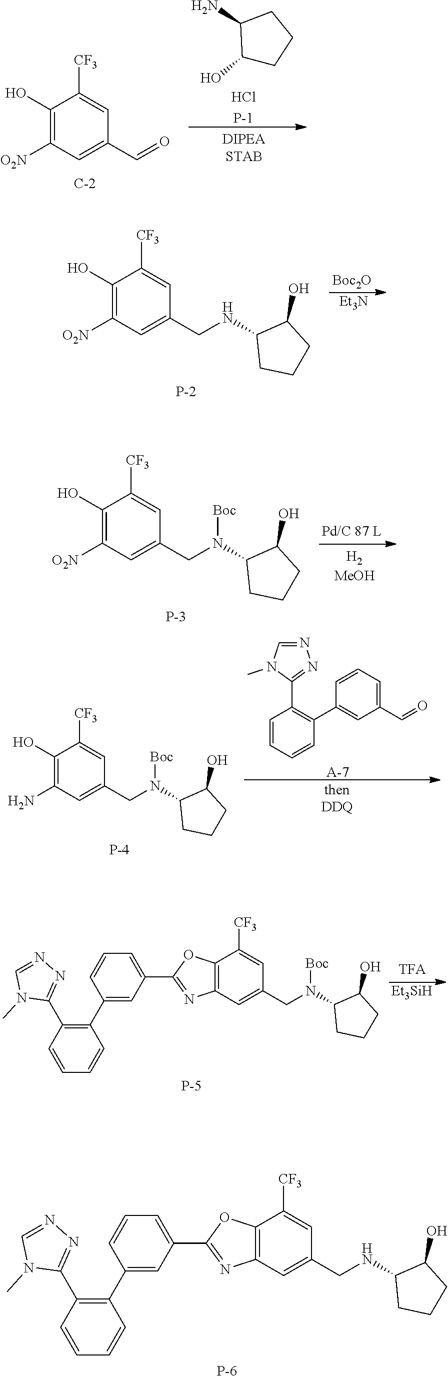

Step 1: 4-((((1S,2S)-2-Hydroxycyclopentyl)amino)methyl)-2-nitro-6-(trifluoromethyl)phenol (P-2). NaBH(OAc)₃ (535.4 mg, 6.0 Eq, 2.526 mmol) was added to a stirred solution of intermediate (C-2) (100.0 mg, 99% Wt, 1 Eq, 421.1 μmol), (1S,2S)-2-aminocyclopentan-1-ol, HCl (P-1) (173.8 mg, 3.0 Eq, 1.263 mmol) and DIPEA (163.3 mg, 217 μL, 3.0 Eq, 1.263 mmol) in THF (5 mL) and stirred at rt overnight. The reaction mixture was concentrated in vacuo. The crude product was purified by chromatography on RP Flash C18 (12 g cartridge, 5-50% (0.7 M ammonia/MeOH)/DCM). The product containing fractions combined and were loaded onto a column of SCX (3 g) in MeOH. The column was washed with MeOH and then the product was eluted with 0.7 M ammonia in MeOH. The resultant mixture was concentrated in vacuo to afford the sub-title compound (P-2) (105 mg, 0.16 mmol, 39%, 50% Purity) as a pale yellow solid. m/z 320.8 (M+H)⁺ (ES+). ¹H NMR (400 MHz, DMSO-d6) δ 7.74 (d, J=2.6 Hz, 1H), 7.31 (d, J=2.6 Hz, 1H), 3.74 (s, 1H), 3.48-3.38 (m, 2H), 2.73 (td, J=6.3, 4.0 Hz, 1H), 1.42-1.14 (m, 6H). Three exchangeable protons not visible.

Step 2: tert-Butyl (4-hydroxy-3-nitro-5-(trifluoromethyl)benzyl)((1S,2S)-2-hydroxycyclopentyl)carbamate (P-3). To a solution of the product from step 1 above (P-2) (105 mg, 50% Wt, 1 Eq, 164 μmol) and di-tert-butyl dicarbonate (39.4 mg, 1.1 Eq, 180 μmol) in DCM (5 mL) and THF (5 mL) was added NEt₃ (33.2 mg, 45.7 μL, 2 Eq, 328 μmol) and stirred at rt for 16 h. The reaction mixture was concentrated under reduced pressure. The crude product was purified by chromatography on silica gel (4 g cartridge, 0-100% EtOAc/isohexane) to afford the sub-title compound (P-3) (80 mg, 95 μmol, 58%, 50% Purity) as a pale yellow oil mixed with tert-butyl ((1S,2S)-2-hydroxycyclopentyl)carbamate (80 mg, 0.20 mmol, 120%, 50% Purity). m/z 419.0 (M−H)⁻ (ES−). ¹H NMR (400 MHz, DMSO-d6) δ 8.16 (s, 1H), 7.91 (s, 1H), 4.73 (s, 1H), 4.36 (d, J=12.3 Hz, 2H), 3.93 (s, 1H), 1.85 (dt, J=14.1, 7.0 Hz, 2H), 1.74 (ddt, J=12.7, 8.9, 6.5 Hz, 2H), 1.55 (dddd, J=14.5, 8.5, 6.9, 2.4 Hz, 2H), 1.37 (s, 9H). Two exchangeable protons not visible.

Step 3: tert-Butyl (3-amino-4-hydroxy-5-(trifluoromethyl)benzyl)((1S,2S)-2-hydroxycyclopentyl)carbamate (P-4). To a solution of the product from step 2 above (P-3) (80 mg, 50% Wt, 1 Eq, 95 μmol) in MeOH (1.5 mL) was added Pd/C type 87L (8 mg, 0.8 Eq, 0.08 mmol). The reaction mixture was stirred at 45° C. with 5 atm of H₂ for 16 h. The catalyst was removed by filtration, and the solvent was removed in vacuo. The crude product was purified by chromatography on silica gel (12 g cartridge, 0-10% MeOH/DCM) to afford the sub-title compound (P-4) (18 mg, 46 μmol, 48%, 99% Purity) as a pale yellow oil. m/z 388.9 (M−H)⁻ (ES−). ¹H NMR (400 MHz, Methanol-d4) δ 6.85 (d, J=2.1 Hz, 1H), 6.76 (d, J=2.1 Hz, 1H), 4.42 (s, 1H), 4.30-4.18 (m, 2H), 3.89 (s, 1H), 1.91 (dq, J=13.4, 6.9, 6.1 Hz, 1H), 1.76 (s, 1H), 1.63 (pd, J=9.4, 7.8, 3.7 Hz, 3H), 1.55-1.32 (m, 10H). Two exchangeable protons not observed.

Step 4: tert-Butyl ((1S,2S)-2-hydroxycyclopentyl)((2-(2′-(4-methyl-4H-1,2,4-triazol-3-yl)-[1,1′-biphenyl]-3-yl)-7-(trifluoromethyl)benzo[d]oxazol-5-yl)methyl)carbamate (P-5). To a solution of intermediate (A-7) (17 mg, 80% Wt, 1 Eq, 52 μmol) in MeOH (1 mL) was added the product from step 3 above (P-4) (20 mg, 99% Wt, 1 Eq, 52 μmol) and the resulting mixture was stirred at 45° C. for 12 h. The reaction mixture was concentrated in vacuo, the residue dissolved in DCM (2 mL) and 4,5-dichloro-3,6-dioxocyclohexa-1,4-diene-1,2-dicarbonitrile (13 mg, 1.1 Eq, 57 μmol) was then added and stirred at rt for 1 h. The resulting mixture was diluted with DCM (5 mL), washed with sat. aq. sol. of NaHCO₃ (2×5 mL) and brine (5 mL). The organic extract was then dried (Na₂SO₄) and concentrated in vacuo. The crude product was purified by chromatography on RP Flash C18 (12 g cartridge, 15-75% MeCN/10 mM ammonium bicarbonate) to afford the sub-title compound (P-5) (23 mg, 36 μmol, 69%, 98% Purity) as a pale brown solid. m/z 634.3 (M+H)⁺ (ES+).

Step 5: (1S,2S)-2-(((2-(2′-(4-Methyl-4H-1,2,4-triazol-3-yl)-[1,1′-biphenyl]-3-yl)-7-(trifluoromethyl)benzo[d]oxazol-5-yl)methyl)amino)cyclopentan-1-ol (P-6). To a solution of the product from step 4 above (P-5) (23.0 mg, 1 Eq, 36.3 μmol) in DCM (0.5 mL) were successively added triethylsilane (8.44 mg, 11.6 μL, 2 Eq, 72.6 μmol), water (3.27 mg, 3.27 μL, 5 Eq, 181 μmol) and TFA (186 mg, 126 μL, 45 Eq, 1.63 mmol) and stirred at rt for 30 min. The mixture was diluted with DCM/water (1:1, 10 mL). The aqueous phase was extracted with DCM (2×2 mL), and the aq. phase basified with 2M NaOH solution. The aq. phase was extracted with DCM (3×5 mL), and the combined organic extracts were washed with brine (5 mL), dried (MgSO₄), filtered and concentrated in vacuo to afford the title compound (P-6) (10 mg, 19 μmol, 51%, 99% Purity) as a tan solid. m/z 534.1 (M+H)⁺ (ES+). ¹H NMR (400 MHz, Methanol-d4) δ 8.40 (s, 1H), 8.23 (dt, J=7.9, 1.4 Hz, 1H), 8.11 (t, J=1.7 Hz, 1H), 8.05 (d, J=1.4 Hz, 1H), 7.87-7.75 (m, 3H), 7.72-7.66 (m, 2H), 7.61 (t, J=7.8 Hz, 1H), 7.53-7.45 (m, 1H), 4.07 (s, 2H), 4.03 (dt, J=6.8, 5.5 Hz, 1H), 3.26 (s, 3H), 2.98 (td, J=7.5, 5.1 Hz, 1H), 2.14-1.96 (m, 2H), 1.82-1.69 (m, 2H), 1.60 (ddt, J=12.5, 8.4, 6.1 Hz, 1H), 1.48 (dq, J=12.8, 7.9 Hz, 1H). Two exchangeable protons not observed.

### Example 12: Synthesis of (S)-2-(2′-(4-Methyl-4H-1,2,4-triazol-3-yl)-[1,1′-biphenyl]-3-yl)-5-((3-methylpiperidin-1-yl)methyl)-7-(trifluoromethyl)benzo[d]oxazole (Q-3)

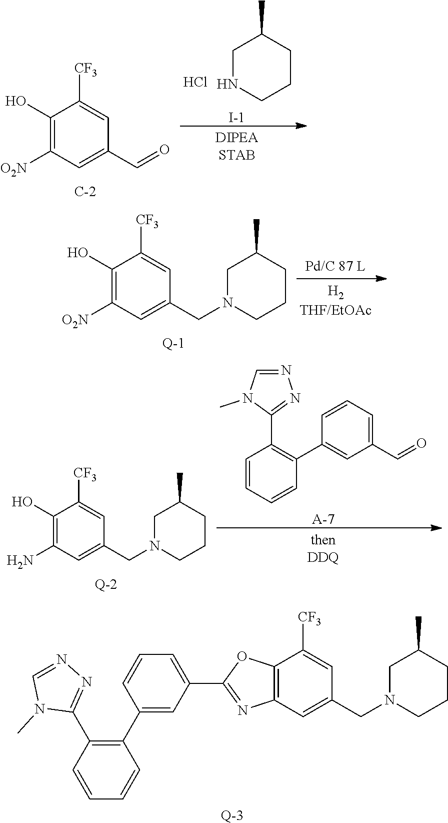

Step 1: (S)-4-((3-Methylpiperidin-1-yl)methyl)-2-nitro-6-(trifluoromethyl)phenol (Q-1). NaBH(OAc)₃ (161 mg, 6.0 Eq, 758 μmol) was added to a stirred solution of intermediate (C-2) (30.0 mg, 99% Wt, 1 Eq, 126 μmol), (S)-3-methylpiperidine, HCl (I-1) (34.3 mg, 2.0 Eq, 253 μmol) and DIPEA (49.0 mg, 65.1 μL, 3.0 Eq, 379 μmol) in THF (1.3 mL) and left at rt overnight. The reaction mixture was concentrated in vacuo. The crude product was purified by chromatography on RP Flash C18 (4 g cartridge, 5-50% (0.7 M ammonia/MeOH)/DCM) to afford the sub-title compound (Q-1) (25 mg, 78 μmol, 62%, 99% Purity) as a pale yellow solid. m/z 319.5 (M+H)⁺ (ES+). ¹H NMR (400 MHz, DMSO-d6) δ 9.01 (s, 1H), 7.94 (d, J=2.6 Hz, 1H), 7.44 (d, J=2.6 Hz, 1H), 4.00 (m, 2H), 3.22 (s, 2H), 2.67 (s, 1H), 2.46-2.34 (m, 1H), 1.86-1.66 (m, 2H), 1.61 (m, 1H), 1.24 (m, 1H), 1.04 (m, 1H), 0.88 (d, J=6.6 Hz, 3H).

Step 2: (S)-2-Amino-4-((3-methylpiperidin-1-yl)methyl)-6-(trifluoromethyl)phenol (Q-2). To a solution of the product from step 1 above (Q-1) (25 mg, 1 Eq, 79 μmol) in THF (1 mL) and EtOAc (1 mL) was added Pd/C type 87L (2.5 mg, 0.30 Eq, 24 μmol). The reaction mixture was stirred at 45° C. with 5 atm of H₂ for 16 h. The catalyst was removed by filtration and the solvent removed in vacuo to afford the sub-title compound (Q-2) (22 mg, 72 μmol, 92%, 95% Purity) as a red oil. m/z 289.2 (M+H)⁺ (ES+). ¹H NMR (400 MHz, Methanol-d4) δ 6.91 (d, J=2.1 Hz, 1H), 6.85 (d, J=2.1 Hz, 1H), 3.51 (s, 2H), 2.94 (dd, J=20.2, 10.4 Hz, 2H), 2.06 (td, J=11.9, 3.1 Hz, 1H), 1.82-1.59 (m, 5H), 1.02-0.92 (m, 1H), 0.90 (d, J=6.0 Hz, 3H). Three exchangeable protons not observed.

Step 3: (S)-2-(2′-(4-Methyl-4H-1,2,4-triazol-3-yl)-[1,1′-biphenyl]-3-yl)-5-((3-methylpiperidin-1-yl)methyl)-7-(trifluoromethyl)benzo[d]oxazole (Q-3). To a solution of intermediate (A-7) (24 mg, 80% Wt, 1 Eq, 72 μmol) in MeOH (1 mL) was added the product from step 2 above (Q-2) (22 mg, 95% Wt, 1 Eq, 72 μmol). The resulting mixture was stirred at 45° C. for 12 h. After concentration under reduced pressure, the residue was dissolved in DCM (2 mL) and 4,5-dichloro-3,6-dioxocyclohexa-1,4-diene-1,2-dicarbonitrile (18 mg, 1.1 Eq, 80 μmol) was then added and stirred at rt for 1 h. The mixture was diluted with DCM (5 mL) and washed sequentially with saturated NaHCO₃ (2×5 mL) and brine (5 mL). The organic layer was dried (Na₂SO₄) and concentrated in vacuo. The crude product was purified by chromatography on RP Flash C18 (4 g cartridge, 15-75% MeCN/10 mM ammonium bicarbonate) to afford the title compound (Q-3) (9 mg, 72 μmol, 20%, 98% Purity) as a pale brown solid. m/z 532.3 (M+H)⁺ (ES+). ¹H NMR (400 MHz, Methanol-d4) δ 8.40 (s, 1H), 8.26-8.20 (m, 1H), 8.11 (s, 1H), 7.99 (s, 1H), 7.86-7.76 (m, 2H), 7.74 (s, 1H), 7.71-7.67 (m, 2H), 7.61 (t, J=7.8 Hz, 1H), 7.49 (dt, J=7.9, 1.4 Hz, 1H), 3.71 (s, 2H), 3.26 (s, 3H), 2.87 (dd, J=14.4, 9.4 Hz, 2H), 2.02 (td, J=11.4, 3.1 Hz, 1H), 1.84-1.56 (m, 5H), 1.04-0.86 (m, 4H).

### Example 13: Synthesis of (1S,2R)-2-(((2-(2′-(4-Methyl-4H-1,2,4-triazol-3-yl)-[1,1′-biphenyl]-3-yl)-7-(trifluoromethyl)benzo[d]oxazol-5-yl)methyl)amino)cyclopentan-1-ol (R-3)

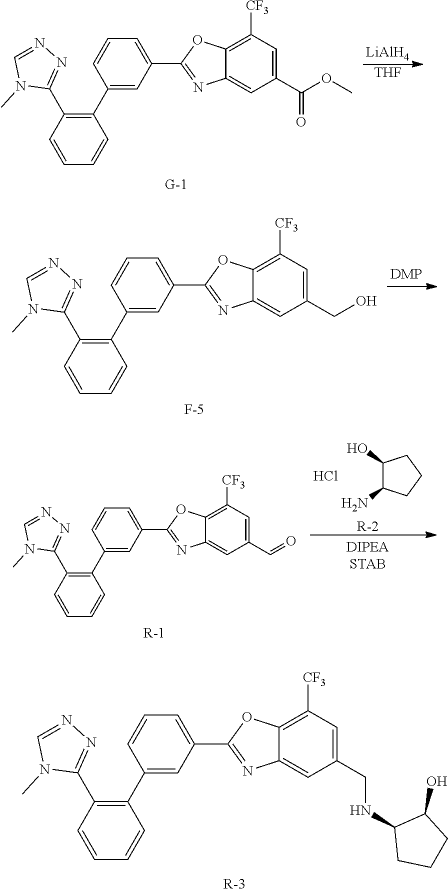

Step 1: (2-(2′-(4-Methyl-4H-1,2,4-triazol-3-yl)-[1,1′-biphenyl]-3-yl)-7-(trifluoromethyl)benzo[d]oxazol-5-yl)methanol (F-5). To a solution of compound (G-1) (60.0 mg, 1 Eq, 125 μmol) in THF (2 mL) was added LiAlH₄ (7.61 mg, 201 μL, 1 molar, 1.6 Eq, 201 μmol) drop-wise at -20° C. and stirred for 1 h at this temperature. The mixture was quenched with water (0.2 mL), and NaOH (aq. 15%, 0.2 mL), and water (0.6 mL) was added at 0° C., followed by anhydrous Na₂SO₄ and THF (15 mL) and the mixture was stirred at 0° C. for 30 min. The mixture was filtered, and the filtrate was concentrated. The crude product was purified by chromatography on RP Flash C18 (12 g cartridge, 15-75% (0.1% formic acid in MeCN)/(0.1% formic acid in water)) to afford the sub-title compound (F-5) (47 mg, 0.10 mmol, 82%, 99% Purity) as a pale white solid. m/z 451.3 (M+H)⁺ (ES+). ¹H NMR (400 MHz, DMSO-d6) δ 8.39 (s, 1H), 8.18-8.11 (m, 1H), 8.04 (s, 1H), 7.95 (t, J=1.8 Hz, 1H), 7.78-7.69 (m, 3H), 7.66-7.61 (m, 2H), 7.59 (t, J=7.8 Hz, 1H), 7.44-7.35 (m, 1H), 5.54 (t, J=5.8 Hz, 1H), 4.69 (d, J=5.8 Hz, 2H), 3.15 (s, 3H).

Step 2: 2-(2′-(4-Methyl-4H-1,2,4-triazol-3-yl)-[1,1′-biphenyl]-3-yl)-7-(trifluoromethyl)benzo[d]oxazole-5-carbaldehyde (R-1). The product from step 1 above (F-5) (30 mg, 1 Eq, 67 μmol) and DMP (42 mg, 1.5 Eq, 0.10 mmol) were stirred in DCM (2 mL) at 40° C. The reaction mixture was diluted with DCM (10 mL) and washed with sat. aq. sol. of NaHCO₃ (10 mL). Organics were separated, dried (MgSO₄), filtered and evaporated to afford the sub-title compound (R-1) (30 mg, 65 μmol, 97%, 97% Purity) as white solid. m/z 449.1 (M+H)⁺ (ES+).

Step 3: (1S,2R)-2-(((2-(2′-(4-Methyl-4H-1,2,4-triazol-3-yl)-[1,1′-biphenyl]-3-yl)-7-(trifluoromethyl)benzo[d]oxazol-5-yl)methyl)amino)cyclopentan-1-ol (R-3). (1S,2R)-2-aminocyclopentan-1-ol, HCl (R-2) (12 mg, 1.3 Eq, 87 μmol) and DIPEA (26 mg, 35 μL, 3.0 Eq, 0.20 mmol) in DCM (1 mL) was added to the product from step 2 above (R-1) (30 mg, 1 Eq, 67 μmol) in DCM (1 mL) followed by NaBH(OAc)₃ (28 mg, 2.0 Eq, 0.13 mmol). The reaction mixture was stirred at rt overnight. The reaction mixture was diluted with EtOAc (25 mL), washed with sat. aq. sol. of NaHCO₃ (2×5 mL) and brine (5 mL). The combined organic extracts were dried (MgSO₄), filtered and concentrated in vacuo. The crude was purified by preparative HPLC (Waters, Basic (0.3% ammonia), Waters XBridge BEH C18 ODB, 5 μm, 30×100 mm column 0-100% MeCN in water) to afford the title compound (R-3) (10 mg, 18 μmol, 27%, 95% Purity) as a white solid. m/z 534.3 (M+H)⁺ (ES+). ¹H NMR (400 MHz, Methanol-d4) δ 8.40 (s, 1H), 8.23 (dt, J=7.9, 1.5 Hz, 1H), 8.11 (t, J=1.8 Hz, 1H), 8.04 (s, 1H), 7.86-7.75 (m, 3H), 7.71-7.67 (m, 2H), 7.60 (t, J=7.8 Hz, 1H), 7.48 (dt, J=7.9, 1.5 Hz, 1H), 4.19 (td, J=4.4, 2.2 Hz, 1H), 4.05 (d, J=13.3 Hz, 1H), 3.97 (d, J=13.2 Hz, 1H), 3.25 (s, 3H), 2.98 (ddd, J=9.3, 7.4, 4.1 Hz, 1H), 2.04-1.39 (m, 6H). Two exchangeable protons not observed.

### Example 14: Synthesis of 2-(4′-Fluoro-2′-(4-methyl-4H-1,2,4-triazol-3-yl)-[1,1′-biphenyl]-3-yl)-7-(trifluoromethyl)benzo[d]oxazole (S-1)

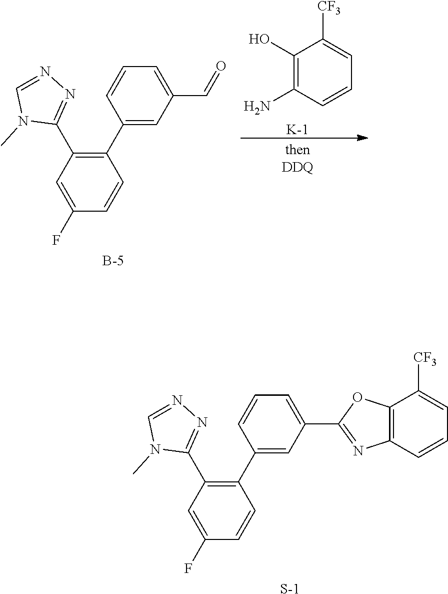

To a solution of intermediate (B-5) (25 mg, 90% Wt, 1 Eq, 80 μmol) in MeOH (1 mL) was added 2-amino-6-(trifluoromethyl)phenol (K-1) (14 mg, 1 Eq, 80 μmol). The resulting mixture was stirred at 45° C. for 16 h. The reaction was concentrated in vacuo, the residue was dissolved in DCM (2 mL) and 4,5-dichloro-3,6-dioxocyclohexa-1,4-diene-1,2-dicarbonitrile (20 mg, 1.1 Eq, 88 μmol) was then added and stirred at rt for 1 h. The resulting mixture was diluted with additional DCM (5 mL) and washed sequentially with sat. aq. sol. of NaHCO₃ (2×5 mL) and brine (5 mL). The organic layer was extracted, dried (phase separator) and concentrated in vacuo. The crude product was purified by chromatography on silica gel (4 g cartridge, 0-100% MeCN/10 mM ammonium bicarbonate) to afford the title compound (S-1) (17.9 mg, 40 μmol, 51%, 99% Purity) as a pale tan solid. m/z 439.0 (M+H)⁺ (ES+). ¹H NMR (400 MHz, DMSO-d6) δ 8.42 (s, 1H), 8.18-8.12 (m, 2H), 7.92 (t, J=1.8 Hz, 1H), 7.83-7.75 (m, 2H), 7.66-7.53 (m, 4H), 7.42-7.36 (m, 1H), 3.22-3.19 (m, 3H).

### Example 15: Synthesis of (1S,2R)-2-(((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)amino)cyclopentan-1-ol (T-5)

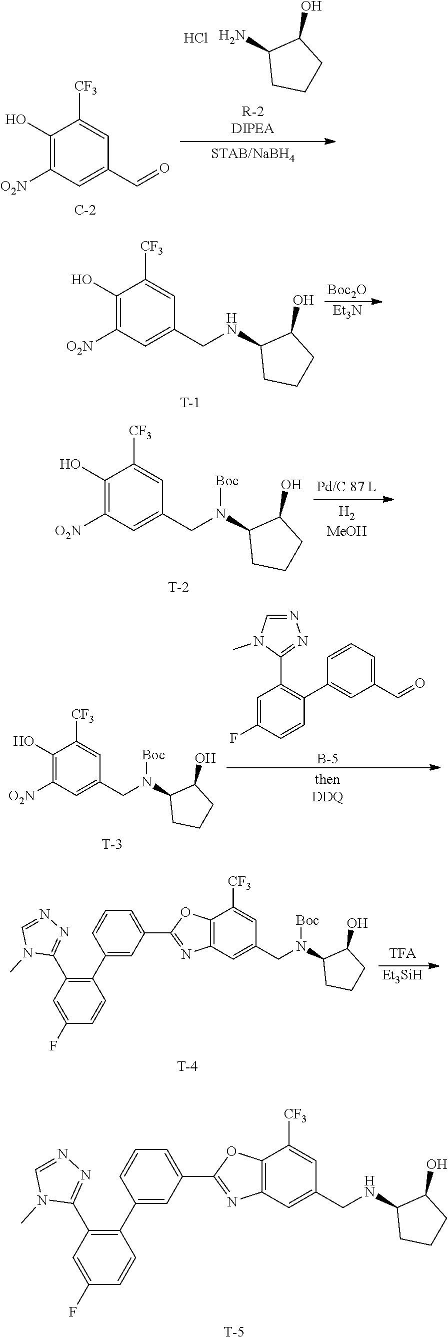

Step 1: 4-((((1R,2S)-2-Hydroxycyclopentyl)amino)methyl)-2-nitro-6-(trifluoromethyl)phenol (T-1). NaBH(OAc)₃ (268 mg, 3 Eq, 1.26 mmol) was added to a stirred solution of intermediate (C-2) (100 mg, 99% Wt, 1 Eq, 421 μmol), (1S,2R)-2-aminocyclopentan-1-ol, HCl (R-2) (63.7 mg, 1.1 Eq, 463 μmol) and DIPEA (163 mg, 0.217 mL, 3.00 Eq, 1.26 mmol) in THF (5 mL) and stirred at rt overnight. NaBH₄ (15.9 mg, 1 Eq, 421 μmol) was added to the reaction mixture and allowed to stir at rt for 16 h. The reaction mixture was quenched with a sat. aq. sol. of NaHCO₃ (10 ml) and extracted with DCM (3×10 mL). The organic extracts were combined, dried (phase separator) and concentrated in vacuo. The crude product was purified by chromatography on RP Flash C18 (12 g cartridge, 0-100% MeCN/10 mM ammonium bicarbonate) to afford the sub-title compound (T-1) (59.5 mg, 0.15 mmol, 35%, 80% Purity) as a bright yellow solid. m/z 321.2 (M+H)⁺ (ES+). ¹H NMR (400 MHz, DMSO-d6) δ 8.01 (d, J=2.7 Hz, 1H), 7.52 (d, J=2.7 Hz, 1H), 4.22 (s, 1H), 3.91 (s, 2H), 1.90 (qd, J=8.6, 4.4 Hz, 1H), 1.84-1.60 (m, 4H), 1.59-1.46 (m, 1H). Two protons masked by water peak and two exchangeable protons not observed.

Step 2: tert-Butyl (4-hydroxy-3-nitro-5-(trifluoromethyl)benzyl)((1S,2S)-2-hydroxycyclopentyl)carbamate (T-2). To a solution of the product from step 1 above (T-1) (59.5 mg, 90% Wt, 1 Eq, 167 μmol) and di-tert-butyl dicarbonate (40.1 mg, 1.1 Eq, 184 μmol) in DCM (2 mL) and THF (2 mL) was added triethylamine (33.8 mg, 46.6 μL, 2 Eq, 334 μmol) and the mixture was stirred at rt for 16 h. The reaction mixture was concentrated in vacuo and the crude product was purified by chromatography on silica gel (12 g cartridge, 0-10% MeOH/DCM) to afford the sub-title compound (T-2) (56.5 mg, 0.11 mmol, 64%, 80% Purity) as a bright yellow oil. m/z 321.0 (M+H-Boc)+(ES+); 419.2 (M−H)⁻ (ES−). ¹H NMR (400 MHz, DMSO-d6) δ 7.40 (d, J=2.3 Hz, 1H), 7.04 (d, J=2.3 Hz, 1H), 3.94 (d, J=16.7 Hz, 1H), 3.71 (d, J=17.0 Hz, 1H), 3.46-3.40 (m, 1H), 3.39-3.30 (m, 1H), 2.40 (q, J=7.3 Hz, 1H), 1.16-0.91 (m, 3H), 0.86-0.71 (m, 2H), 0.62 (s, 9H). Two exchangeable protons not observed.

Step 3: tert-Butyl (3-amino-4-hydroxy-5-(trifluoromethyl)benzyl)((1R,2S)-2-hydroxycyclopentyl)carbamate (T-3). To a solution of the product from step 2 above (T-2) (56.5 mg, 80% Wt, 1 Eq, 108 μmol) in MeOH (2 mL) was added Pd/C type 87L (1.48 mg, 0.13 Eq, 14.0 μmol). The reaction mixture was stirred at rt with 5 atm of H₂ for 16 h. The catalyst was removed by filtration and the solvent was removed in vacuo to afford the sub-title compound (T-3) (48.6 mg, 0.11 mmol, 100%, 88% Purity) as a dark brown solid. m/z 291.2 (M+H-Boc)+(ES+); 389.3 (M−H)⁻ (ES−).

Step 4: tert-Butyl ((2-(4′-fluoro-2′-(4-methyl-4H-1,2,4-triazol-3-yl)-[1,1′-biphenyl]-3-yl)-7-(trifluoromethyl)benzo[d]oxazol-5-yl)methyl)((1S,2S)-2-hydroxycyclopentyl)carbamate (T-4). To a solution of intermediate (B-5) (31.1 mg, 90% Wt, 1 Eq, 99.6 μmol) in MeOH (1 mL) was added the product from step 3 above (T-3) (48.6 mg, 80% Wt, 1 Eq, 99.6 μmol). The resulting mixture was stirred at 45° C. for 16 h. The reaction mixture was concentrated in vacuo, the residue was dissolved in DCM (2 mL) and 4,5-dichloro-3,6-dioxocyclohexa-1,4-diene-1,2-dicarbonitrile (24.9 mg, 1.1 Eq, 110 μmol) was added and allowed to stir at rt for 1 h. The resulting mixture was diluted with additional DCM (5 mL) and washed sequentially with sat. aq. sol. of NaHCO₃ (5 mL×2) and brine (5 mL). The organic extract was extracted, dried (phase separator) and concentrated in vacuo. The crude product was purified by chromatography on silica gel (4 g cartridge, 0-10% MeOH/DCM) to afford the sub-title compound (T-4) (68.5 mg, 63 μmol, 63%, 60% Purity) as a dark orange solid. m/z 652.4 (M+H)⁺ (ES+).

Step 5: (1S,2R)-2-(((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)amino)cyclopentan-1-ol (T-5). To a solution of the product from step 4 above (T-4) (68.5 mg, 60% Wt, 1 Eq, 63.1 μmol) in DCM (0.5 mL) were successively added triethylsilane (14.7 mg, 20.1 μL, 2 Eq, 126 μmol), water (5.68 mg, 5.68 μL, 5 Eq, 315 μmol) and trifluoroacetic acid (324 mg, 219 μL, 45 Eq, 2.84 mmol). This mixture was stirred at rt for 3 h. The reaction mixture was concentrated in vacuo and the crude product was purified by preparative HPLC (Waters, Basic (0.3% Ammonia), Basic, Waters X-Bridge BEH C18 ODB Prep-C18, 5 μm, 30×100 mm column, 0-100% MeCN in Water) to afford the title compound (T-5) (10.39 mg, 18 μmol, 28%, 95% Purity) as a pale pink solid. m/z 552.1 (M+H)⁺ (ES+). ¹H NMR (400 MHz, DMSO-d6) δ 8.42 (s, 1H), 8.15-8.11 (m, 1H), 8.09 (s, 1H), 7.93-7.89 (m, 1H), 7.81-7.74 (m, 2H), 7.65-7.52 (m, 3H), 7.40-7.36 (m, 1H), 4.39 (d, J=3.5 Hz, 1H), 3.99-3.85 (m, 3H), 3.20 (s, 3H), 2.82-2.75 (m, 1H), 1.74-1.53 (m, 3H), 1.47-1.34 (m, 2H). Two exchangeable protons not observed.

### Example 16: Synthesis of (S)-2-(4′-Fluoro-2′-(4-methyl-4H-1,2,4-triazol-3-yl)-[1,1′-biphenyl]-3-yl)-5-((3-methylpiperidin-1-yl)methyl)-7-(trifluoromethyl)benzo[d]oxazole (U-1)

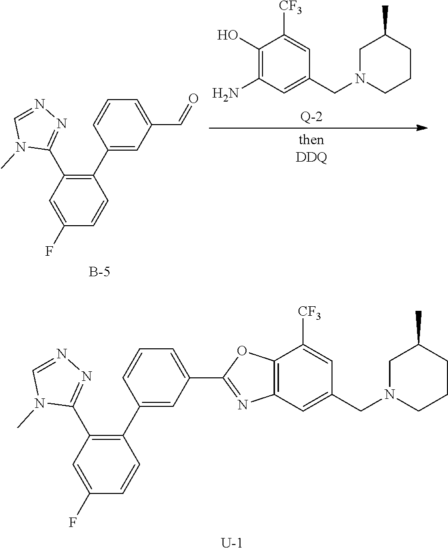

To a solution of intermediate (B-5) (25 mg, 90% Wt, 1 Eq, 80 μmol) in MeOH (1 mL) was added intermediate (Q-2) (29 mg, 80% Wt, 1 Eq, 80 μmol). The resulting mixture was stirred at 45° C. for 12 h. The reaction mixture was concentrated in vacuo, and the residue was dissolved in DCM (2 mL) and 4,5-dichloro-3,6-dioxocyclohexa-1,4-diene-1,2-dicarbonitrile (18 mg, 1 Eq, 80 μmol) was added. After stirring at rt for 1 h, the resulting mixture was diluted with additional DCM (5 mL) and washed sequentially with sat. aq. sol. of NaHCO₃ (2×5 mL) and brine (5 mL). The organic extract was dried (phase separator) and concentrated in vacuo. The crude product was purified by chromatography on RP Flash C18 (4 g cartridge, 0-100% MeCN/10 mM ammonium bicarbonate) to afford the title compound (U-1) (12.8 mg, 23 μmol, 28%, 97% Purity) as an orange solid. m/z 550.2 (M+H)⁺ (ES+). ¹H NMR (400 MHz, DMSO-d6) δ 8.42 (s, 1H), 8.15-8.11 (m, 1H), 8.02 (s, 1H), 7.90 (t, J=1.8 Hz, 1H), 7.80-7.75 (m, 1H), 7.70 (s, 1H), 7.64-7.53 (m, 3H), 7.41-7.36 (m, 1H), 3.64 (s, 2H), 3.22-3.19 (m, 3H), 2.72 (d, J=8.1 Hz, 2H), 1.97-1.88 (m, 1H), 1.69-1.55 (m, 4H), 1.55-1.42 (m, 1H), 0.92-0.84 (m, 1H), 0.82 (d, J=6.1 Hz, 3H).

### Example 17: Synthesis of (R)-2-(4′-Fluoro-2′-(4-methyl-4H-1,2,4-triazol-3-yl)-[1,1′-biphenyl]-3-yl)-5-((2-methylmorpholino)methyl)-7-(trifluoromethyl)benzo[d]oxazole (V-1)

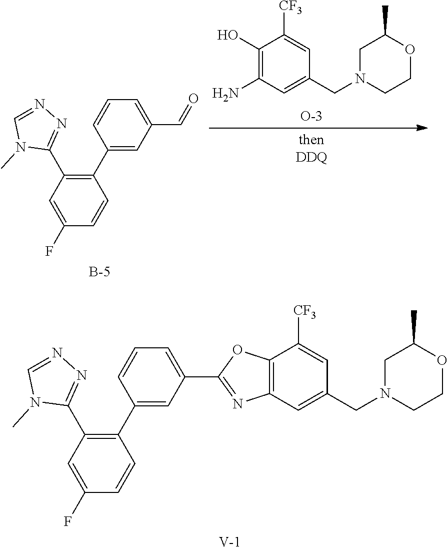

To a solution of intermediate (B-5) (25 mg, 90% Wt, 1 Eq, 80 μmol) in MeOH (1 mL) was added intermediate (0-3) (28 mg, 80% Wt, 1 Eq, 80 μmol) and stirred at 45° C. for 16 h. The reaction mixture was concentrated in vacuo, the residue was dissolved in DCM (2 mL) and 4,5-dichloro-3,6-dioxocyclohexa-1,4-diene-1,2-dicarbonitrile (20 mg, 1.1 Eq, 88 μmol) was added and allowed to stir at rt for 1 h. The reaction mixture was diluted with DCM (5 mL) and washed sequentially with sat. aq. sol. of NaHCO₃ (2×5 mL) and brine (5 mL). The organic extract was dried (phase separator) and concentrated in vacuo. The crude product was purified by chromatography on RP Flash C18 (4 g cartridge, 0-100% MeCN/10 mM ammonium bicarbonate) to afford the title compound (V-1) (14.4 mg, 25 μmol, 32%, 97% Purity) as a dark tan solid. m/z 552.1 (M+H)⁺ (ES+). ¹H NMR (400 MHz, DMSO-d6) δ 8.42 (s, 1H), 8.16-8.11 (m, 1H), 8.05 (s, 1H), 7.90 (t, J=1.8 Hz, 1H), 7.80-7.73 (m, 1H), 7.72 (s, 1H), 7.64-7.53 (m, 3H), 7.42-7.37 (m, 1H), 3.76-3.71 (m, 1H), 3.67 (s, 1H), 3.58-3.47 (m, 2H), 3.20 (s, 3H), 2.75-2.61 (m, 3H), 2.14-2.04 (m, 1H), 1.79 (t, J=10.5 Hz, 1H), 1.03 (d, J=6.3 Hz, 3H).

### Example 18: Synthesis of (1R,2R)-2-(((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)amino)cyclopentan-1-ol (W-2)

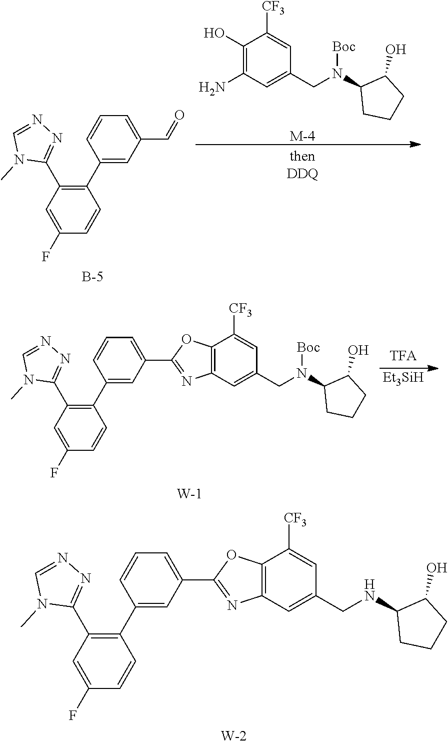

Step 1: tert-Butyl ((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)((1S,2R)-2-hydroxycyclopentyl)carbamate (W-1). To a solution of intermediate (B-5) (20 mg, 90% Wt, 1 Eq, 64 μmol) in MeOH (1 mL) was added intermediate (M-4) (31 mg, 80% Wt, 1 Eq, 64 μmol) and the mixture was stirred at 45° C. for 16 h. The reaction mixture was concentrated in vacuo, the residue was dissolved in DCM (2 mL) and 4,5-dichloro-3,6-dioxocyclohexa-1,4-diene-1,2-dicarbonitrile (16 mg, 1.1 Eq, 70 μmol) was added and allowed to stir at rt for 1 h. The mixture was diluted with DCM (5 mL) and washed sequentially with sat. aq. sol. of NaHCO₃ (2×5 mL) and brine (5 mL). The organic extract was, dried (phase separator) and concentrated in vacuo. The crude product was purified by chromatography on silica gel (4 g cartridge, 0-10% MeOH/DCM) to afford the sub-title compound (W-1) (26 mg, 36 μmol, 56%, 90% Purity) as a dark yellow oil. m/z 595.6 (M+H-tBu)⁺ (ES+).

Step 2: (1R,2R)-2-(((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)amino)cyclopentan-1-ol (W-2). To a solution of the product from step 1 above (W-1) (26.0 mg, 92% Wt, 1 Eq, 36.7 μmol) in DCM (0.5 mL) were successively added triethylsilane (8.54 mg, 11.7 μL, 2 Eq, 73.4 μmol), water (3.31 mg, 3.31 μL, 5 Eq, 184 μmol) and trifluoroacetic acid (188 mg, 127 μL, 45 Eq, 1.65 mmol). The mixture was stirred at rt for 3 h. The reaction mixture was concentrated under reduced pressure and the crude product was purified by preparative HPLC (Waters, Basic (0.3% ammonia), Basic, Waters X-Bridge BEH C18 ODB Prep-C18, 5 μm, 30×100 mm column, 0-100% MeCN in Water) to afford the title compound (W-2) (8.94 mg, 15 μmol, 42%, 95% Purity) as a pale yellow solid. m/z 552.4 (M+H)⁺ (ES+). ¹H NMR (400 MHz, DMSO-d6) δ 8.42 (s, 1H), 8.15-8.11 (m, 1H), 8.07 (s, 1H), 7.91-7.89 (m, 1H), 7.80-7.75 (m, 2H), 7.64-7.52 (m, 3H), 7.40-7.36 (m, 1H), 4.51 (d, J=4.2 Hz, 1H), 3.90 (s, 2H), 3.83-3.78 (m, 1H), 3.20 (s, 3H), 1.88-1.77 (m, 2H), 1.62-1.52 (m, 2H), 1.45-1.25 (m, 2H). Two exchangeable protons not observed.

### Example 19: Synthesis of (2-(4′-Fluoro-2′-(4-methyl-4H-1,2,4-triazol-3-yl)-[1,1′-biphenyl]-3-yl)-7-(trifluoromethyl)benzo[d]oxazol-5-yl)methanol (X-2)

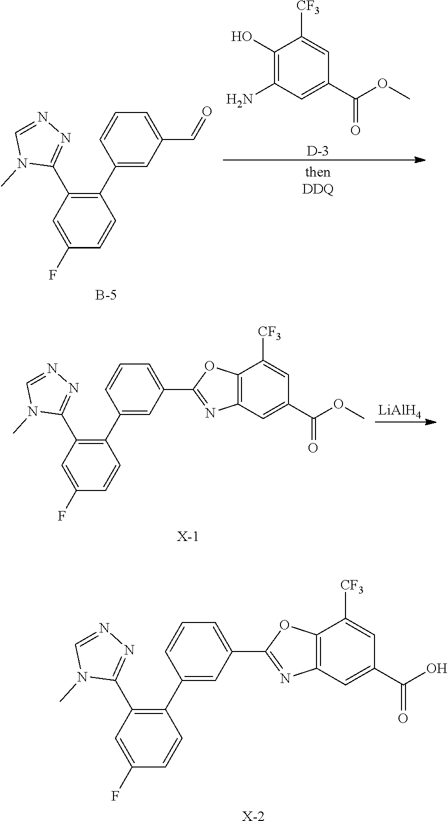

Step 1: Methyl 2-(4′-fluoro-2′-(4-methyl-4H-1,2,4-triazol-3-yl)-[1,1′-biphenyl]-3-yl)-7-(trifluoromethyl)benzo[d]oxazole-5-carboxylate (X-1). To a solution of intermediate (B-5) (150 mg, 1 Eq, 533 μmol) in MeOH (2 mL) was added intermediate (D-3) (139 mg, 90% Wt, 1 Eq, 533 μmol). The resulting mixture was stirred at 45° C. for 12 h. After concentration in vacuo, the residue was dissolved in DCM (2 mL) and 4,5-dichloro-3,6-dioxocyclohexa-1,4-diene-1,2-dicarbonitrile (133 mg, 1.1 Eq, 587 μmol) was then added. After stirring at rt for 1 h, the resulting mixture was diluted with DCM (5 mL) and washed sequentially with sat. aq. sol. of NaHCO₃ (2×5 mL) and brine (5 mL). The organic layer was dried (Na₂SO₄), filtered and concentrated in vacuo. The crude product was purified by chromatography on silica gel (40 g cartridge, 0-10% MeOH/DCM) to afford the sub-title compound (X-1) (193 mg, 0.37 mmol, 70%, 96% Purity) as a pale brown solid. m/z 497.3 (M+H)⁺ (ES+). ¹H NMR (400 MHz, DMSO-d6) δ 8.62 (d, J=1.5 Hz, 1H), 8.42 (s, 1H), 8.26 (d, J=1.4 Hz, 1H), 8.16 (dt, J=7.9, 1.3 Hz, 1H), 7.94 (t, J=1.8 Hz, 1H), 7.78 (dd, J=8.6, 5.6 Hz, 1H), 7.66-7.52 (m, 3H), 7.41 (dt, J=8.0, 1.3 Hz, 1H), 3.95 (s, 3H), 3.22 (s, 3H).

Step 2: (2-(4′-Fluoro-2′-(4-methyl-4H-1,2,4-triazol-3-yl)-[1,1′-biphenyl]-3-yl)-7-(trifluoromethyl)benzo[d]oxazol-5-yl)methanol (X-2). To a solution of the product from step 1 above (X-1) (100.0 mg, 1 Eq, 201.4 μmol) in THF (2 mL) was added LiAlH₄ (15.29 mg, 402.9 μL, 1 molar, 2.0 Eq, 402.9 μmol) drop-wise at −20° C. and the mixture was stirred for 1 h at this temperature. The mixture was quenched with water (0.2 mL), and NaOH (aq. 15%, 0.2 mL). Additional water (0.6 mL) was added at 0° C. followed by anhydrous Na₂SO₄ and THF (15 mL). The mixture was stirred at 0° C. for 30 min. The mixture was filtered, and the filtrate was concentrated in vacuo. The crude product was purified by chromatography on RP Flash C18 (12 g cartridge, 15-75% (0.1% formic acid in MeCN)/(0.1% formic acid in water)) to afford the title compound (X-2) (43 mg, 91 μmol, 45%, 99% Purity) as a pale white solid. m/z 469.3 (M+H)⁺ (ES+). ¹H NMR (400 MHz, DMSO-d6) δ 8.42 (s, 1H), 8.13 (dt, J=7.9, 1.4 Hz, 1H), 8.04 (s, 1H), 7.92 (t, J=1.8 Hz, 1H), 7.78 (dd, J=8.6, 5.6 Hz, 1H), 7.74 (s, 1H), 7.65-7.53 (m, 3H), 7.37 (dt, J=8.0, 1.4 Hz, 1H), 5.54 (t, J=5.8 Hz, 1H), 4.69 (d, J=5.5 Hz, 2H), 3.21 (s, 3H).

### Example 20: Synthesis of 1-((((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)amino)methyl)cyclobutan-1-ol (Y-3)

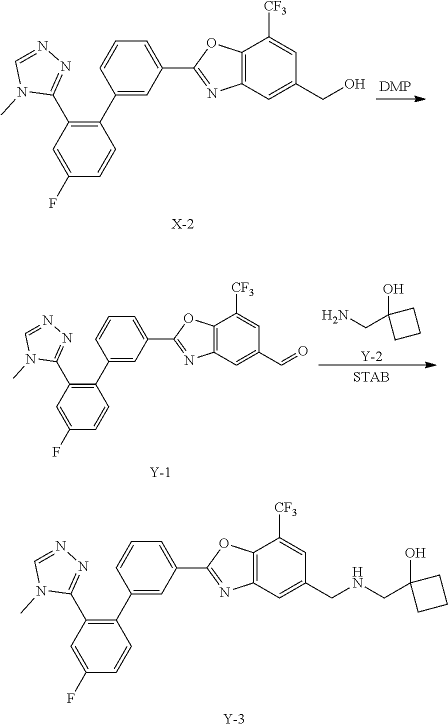

Step 1: 2-(4′-Fluoro-2′-(4-methyl-4H-1,2,4-triazol-3-yl)-[1,1′-biphenyl]-3-yl)-7-(trifluoromethyl)benzo[d]oxazole-5-carbaldehyde (Y-1). Compound (X-2) (77.0 mg, 1 Eq, 164 μmol) and Dess-Martin periodinane (105 mg, 1.5 Eq, 247 μmol) were stirred in DCM (2 mL) at 40° C. The reaction mixture was diluted with DCM (10 mL) and washed with sat. aq. NaHCO₃ (10 mL) and brine. Organics were separated, dried (MgSO₄), filtered and evaporated to afford the sub-title compound (Y-1) (77 mg, 0.15 mmol, 92%, 92% Purity) as white solid. m/z 467.0 (M+H)⁺ (ES+).

Step 2: 1-((((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)amino)methyl)cyclobutan-1-ol (Y-3). A solution of the product from step 1 above (Y-2) (25 mg, 1 Eq, 54 μmol) and 1-(aminomethyl)cyclobutan-1-ol (Y-2) (8.1 mg, 1.5 Eq, 80 μmol) in CHCl₃ (1 mL) was stirred 1 h at rt, then NaBH(OAc)₃ (28 mg, 2.5 Eq, 0.13 mmol) was added. The reaction mixture was stirred at rt overnight. An additional 1 eq. of NaBH(OAc)₃ was added and the mixture was stirred 3h at rt. The reaction mixture was diluted with EtOAc (25 mL) and transferred into a separating funnel. The layer was washed with sat. aq. sol. of NaHCO₃ (2×5 mL) and brine. The combined organic layers were collected, dried (MgSO₄), filtered and concentrated in vacuo. The crude product was purified by chromatography on RP Flash C18 (12 g cartridge, 15-75% MeCN/10 mM ammonium bicarbonate) to afford the title compound (Y-3) (13 mg, 23 μmol, 42%, 96% Purity) as a white solid. m/z 552.1 (M+H)⁺ (ES+). ¹H NMR (400 MHz, Methanol-d4) δ 8.41 (s, 1H), 8.24 (dt, J=7.9, 1.4 Hz, 1H), 8.09 (t, J=1.7 Hz, 1H), 8.07-8.03 (m, 1H), 7.84-7.78 (m, 2H), 7.64-7.54 (m, 2H), 7.54-7.44 (m, 2H), 4.05 (s, 2H), 3.28 (s, 3H), 2.76 (s, 2H), 2.23-1.99 (m, 4H), 1.85-1.71 (m, 1H), 1.55 (dt, J=11.3, 8.9 Hz, 1H). Exchangeable protons not observed.

### Example 21: Synthesis of (3-(((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)amino)oxetan-3-yl)methanol (Z-2)

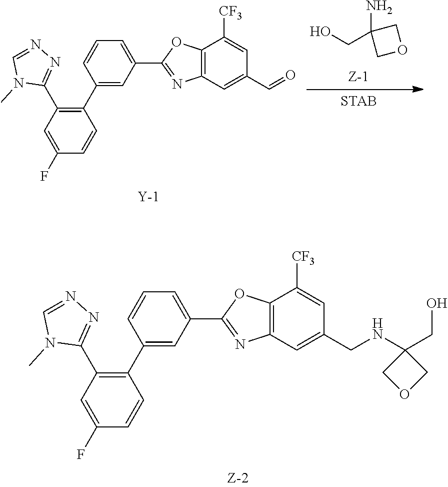

A solution of intermediate (Y-1) (25 mg, 1 Eq, 54 μmol) and (3-aminooxetan-3-yl)methanol (Z-1) (8.3 mg, 1.5 Eq, 80 μmol) in CHCl₃ (1 mL) was stirred 1 h at rt, then NaBH(OAc)₃ (28 mg, 2.5 Eq, 0.13 mmol) was added. The reaction mixture was stirred at rt overnight. An additional 1 eq. of NaBH(OAc)₃ was added and the mixture was stirred 3h at rt. The reaction mixture was diluted with EtOAc (25 mL) and transferred into a separating funnel. The layer was washed with sat. aq. sol. of NaHCO₃ (2×5 mL) and brine. The combined organic layers were collected, dried (MgSO₄), filtered and concentrated in vacuo. The crude product was purified by chromatography on RP Flash C18 (12 g cartridge, 15-75% MeCN/10 mM ammonium bicarbonate) to afford an impure product (>85% purity) which was loaded onto a column of SCX (500 mg) in MeOH. The column was washed with MeOH and then the product was eluted with 0.7 M ammonia in MeOH. The resultant mixture was concentrated in vacuo to afford the title compound (Z-2) (14 mg, 25 μmol, 46%, 97% Purity) as a clear white solid. m/z 554.2 (M+H)⁺ (ES+). ¹H NMR (400 MHz, Methanol-d4) δ 8.41 (s, 1H), 8.24 (d, J=7.8 Hz, 1H), 8.09 (s, 2H), 7.83 (d, J=11.0 Hz, 2H), 7.66-7.54 (m, 2H), 7.48 (t, J=11.1 Hz, 2H), 4.68 (d, J=6.4 Hz, 2H), 4.51 (d, J=6.4 Hz, 2H), 4.06 (s, 2H), 3.92 (s, 2H), 3.27 (s, 3H). Exchangeable protons not observed.

### Example 22: Synthesis of N-((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)-2-methoxyethan-1-amine (AA-2)

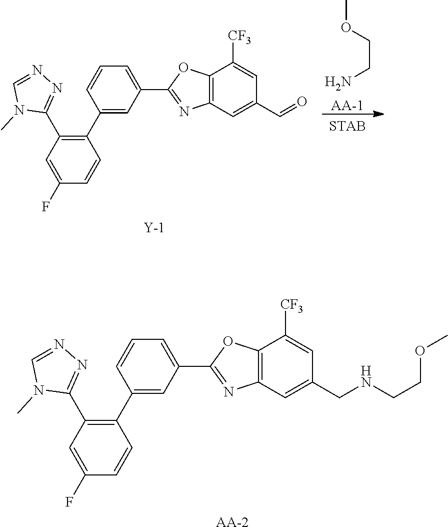

A solution of intermediate (Y-1) (20 mg, 1 Eq, 43 μmol) and 2-methoxyethan-1-amine (AA-1) (5.8 mg, 6.7 μL, 1.8 Eq, 77 μmol) in CHCl₃ (1 mL) was stirred 1 h at rt, then NaBH(OAc)₃ (27 mg, 3.0 Eq, 0.13 mmol) was added. The reaction mixture was stirred at rt overnight. The reaction mixture was diluted with EtOAc (25 mL) and transferred into a separating funnel. The layer was washed with sat. aq. sol. of NaHCO₃ (2×5 mL) and brine. The combined organic layers were collected, dried (MgSO₄), filtered and concentrated in vacuo. The crude product was purified by chromatography on silica gel (12 g cartridge, 0-10% MeOH/DCM) to afford the title compound (AA-2) (16 mg, 30 μmol, 70%, 99% Purity) as a clear white solid. m/z 526.3 (M+H)⁺ (ES+). ¹H NMR (400 MHz, Methanol-d4) δ 8.41 (s, 1H), 8.24 (dt, J=7.9, 1.4 Hz, 1H), 8.09 (t, J=1.8 Hz, 1H), 8.03 (s, 1H), 7.85-7.76 (m, 2H), 7.66-7.53 (m, 2H), 7.53-7.43 (m, 2H), 4.03 (s, 2H), 3.57 (t, J=5.3 Hz, 2H), 3.39 (s, 3H), 3.27 (s, 3H), 2.85 (t, J=5.3 Hz, 2H). Exchangeable proton not visible.

### Example 23: Synthesis of (S)-2-(4′-Fluoro-2′-(4-methyl-4H-1,2,4-triazol-3-yl)-[1,1′-biphenyl]-3-yl)-5-((3-methoxypyrrolidin-1-yl)methyl)-7-(trifluoromethyl)benzo[d]oxazole (AB-2)

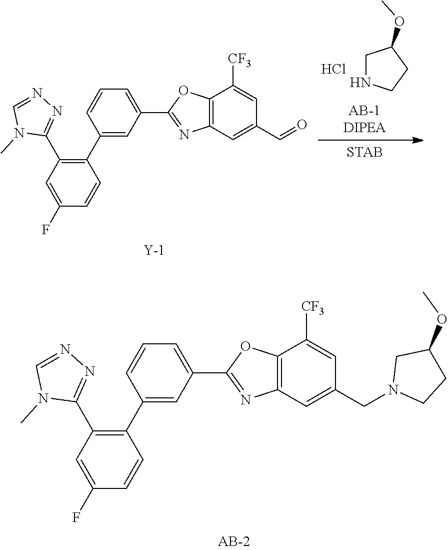

A solution of intermediate (Y-1) (20 mg, 1 Eq, 43 μmol), DIPEA (14 mg, 19 μL, 2.5 Eq, 0.11 mmol) and (S)-3-methoxypyrrolidine, HCl (AB-1) (11 mg, 1.8 Eq, 77 μmol) in CHCl₃ (1 mL) was stirred 1 h at rt, then NaBH(OAc)₃ (27 mg, 3.0 Eq, 0.13 mmol) was added. The reaction mixture was stirred at rt overnight. The reaction mixture was diluted with EtOAc (25 mL) and transferred into a separating funnel. The layer was washed with sat. aq. sol. of NaHCO₃ (2×5 mL) and brine. The combined organic layers were collected, dried (MgSO₄), filtered and concentrated in vacuo. The crude product was purified by chromatography on silica gel (12 g cartridge, 0-10% MeOH/DCM) to afford the title compound (13 mg, 22 μmol, 52%, 95% Purity) as a clear white solid. m/z 552.3 (M+H)⁺ (ES+). ¹H NMR (400 MHz, Methanol-d4) δ 8.41 (s, 1H), 8.23 (dt, J=7.9, 1.4 Hz, 1H), 8.09 (t, J=1.8 Hz, 1H), 8.01 (d, J=1.5 Hz, 1H), 7.81 (dd, J=8.6, 5.5 Hz, 1H), 7.77 (s, 1H), 7.64-7.54 (m, 2H), 7.52-7.44 (m, 2H), 4.01 (dp, J=8.8, 3.1 Hz, 1H), 3.88 (d, J=13.1 Hz, 1H), 3.82 (d, J=13.1 Hz, 1H), 3.30 (s, 3H), 3.27 (s, 3H), 2.78 (dd, J=10.3, 6.1 Hz, 2H), 2.69 (dd, J=10.4, 3.2 Hz, 1H), 2.56 (td, J=8.7, 6.3 Hz, 1H), 2.22-2.09 (m, 1H), 1.94-1.79 (m, 1H).

### Example 24: Synthesis of 3-((((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)amino)methyl)oxetan-3-ol (AC-2)

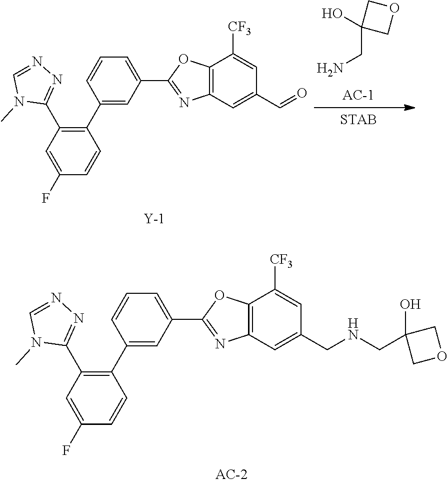

A solution of intermediate (Y-1) (25 mg, 1 Eq, 54 μmol) and 3-(aminomethyl)oxetan-3-ol (AC-1) (8.3 mg, 1.5 Eq, 80 μmol) in CHCl₃ (1 mL) was stirred 1h at rt, then NaBH(OAc)₃ (28 mg, 2.5 Eq, 0.13 mmol) was added. The reaction mixture was stirred at rt overnight. An additional 1 eq. of NaBH(OAc)₃ was added and the mixture was stirred 3h at rt. The reaction mixture was diluted with EtOAc (25 mL) and transferred into a separating funnel. The layer was washed with sat. aq. sol. of NaHCO₃ (2×5 mL) and brine. The combined organic layers were collected, dried (MgSO₄), filtered and concentrated in vacuo. The crude product was purified by chromatography on RP Flash C18 (12 g cartridge, 15-75% MeCN/10 mM ammonium bicarbonate) to afford an impure product (>85% purity) which was loaded onto a column of SCX (500 mg) in MeOH. The column was washed with MeOH and then the product was eluted with 0.7 M ammonia in MeOH. The resultant mixture was concentrated in vacuo to afford a 92% pure product. This product was purified by chromatography on silica gel (12 g cartridge, 0-10% MeOH/DCM) to afford the title compound (AC-2) (12 mg, 21 μmol, 39%, 97% Purity) as a clear white solid. m/z 554.10 (M+H)⁺ (ES+). ¹H NMR (400 MHz, Methanol-d4) δ 8.41 (s, 1H), 8.24 (dt, J=8.0, 1.3 Hz, 1H), 8.09 (t, J=1.8 Hz, 1H), 8.05 (d, J=1.5 Hz, 1H), 7.84-7.77 (m, 2H), 7.64-7.53 (m, 2H), 7.53-7.43 (m, 2H), 4.59 (d, J=6.7 Hz, 2H), 4.56-4.52 (m, 2H), 4.05 (s, 2H), 3.27 (s, 3H), 2.97 (s, 2H). Exchangeable protons not visible.

### Example 25: Synthesis of (S)-1-((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)pyrrolidin-3-ol (AD-2)

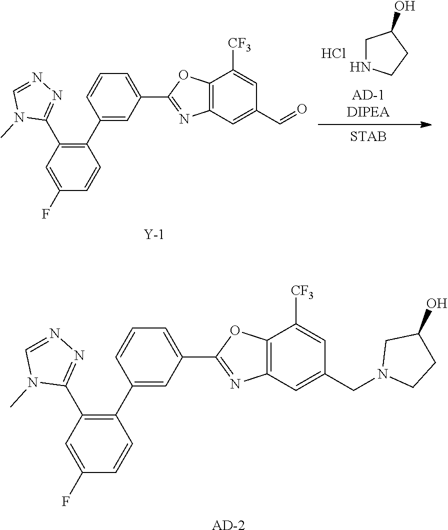

A solution of intermediate (Y-1) (20 mg, 1 Eq, 43 μmol), DIPEA (14 mg, 19 μL, 2.5 Eq, 0.11 mmol) and (S)-pyrrolidin-3-ol, HCl (AD-1) (9.5 mg, 1.8 Eq, 77 μmol) in CHCl₃ (1 mL) was stirred 1 h at rt, then NaBH(OAc)₃ (27 mg, 3.0 Eq, 0.13 mmol) was added. The reaction mixture was stirred at rt overnight. The reaction mixture was diluted with EtOAc (25 mL) and transferred into a separating funnel. The layer was washed with sat. aq. sol. of NaHCO₃ (2×5 mL) and brine. The combined organic layers were collected, dried (MgSO₄), filtered and concentrated in vacuo. The crude product was purified by chromatography on silica gel (12 g cartridge, 0-10% MeOH/DCM) to afford the title compound (AD-2) (14 mg, 26 μmol, 60%, 99% Purity) as a clear white solid. m/z 538.2 (M+H)⁺ (ES+). ¹H NMR (400 MHz, Methanol-d4) δ 8.41 (s, 1H), 8.23 (dt, J=7.9, 1.4 Hz, 1H), 8.09 (t, J=1.7 Hz, 1H), 8.02 (d, J=1.5 Hz, 1H), 7.81 (dd, J=8.7, 5.4 Hz, 1H), 7.77 (s, 1H), 7.63-7.53 (m, 2H), 7.53-7.43 (m, 2H), 4.40 (dp, J=9.1, 3.2 Hz, 1H), 3.90 (d, J=13.1 Hz, 1H), 3.84 (d, J=13.0 Hz, 1H), 3.28 (s, 3H), 2.84 (dd, J=10.2, 5.9 Hz, 2H), 2.70-2.53 (m, 2H), 2.21 (dq, J=14.5, 7.5 Hz, 1H), 1.78 (dddd, J=13.4, 8.2, 5.6, 2.9 Hz, 1H). Exchangeable proton not visible.

### Example 26: Synthesis of 1-(((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)amino)-2-methylpropan-2-ol (AE-2)

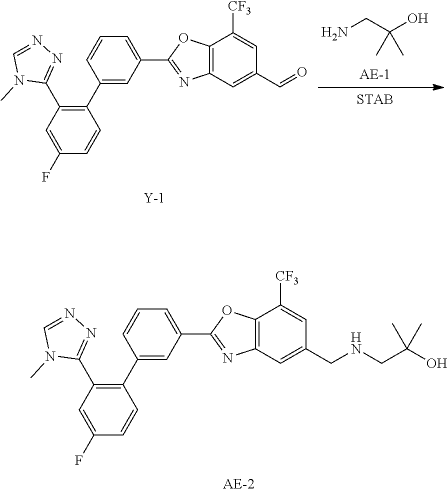

A solution of intermediate (Y-1) (22.5 mg, 80% Wt, 1 Eq, 38.6 μmol) and 1-amino-2-methylpropan-2-ol (AE-1) (3.44 mg, 3.7 μL, 1 Eq, 38.6 μmol) in CHCl₃ (1 mL) was stirred 1 h at rt, then NaBH(OAc)₃ (24.5 mg, 3.0 Eq, 116 μmol) was added. The reaction mixture was stirred at rt overnight. The reaction mixture was diluted with EtOAc (10 mL), washed with sat. aq. sol. of NaHCO₃ (2×5 mL) and brine (5 mL). The organic layer was dried (MgSO₄), filtered and concentrated in vacuo. The crude product was purified by chromatography on silica gel (12 g cartridge, 0-10% MeOH/DCM) to afford the title compound (AE-2) (8.7 mg, 15 μmol, 40%, 95% Purity) as an off white solid. m/z 540.1 (M+H)⁺ (ES+). ¹H NMR (400 MHz, DMSO-d6) δ 8.42 (s, 1H), 8.16-8.11 (m, 1H), 8.09 (s, 1H), 7.93-7.89 (m, 1H), 7.82-7.78 (m, 1H), 7.78-7.75 (m, 1H), 7.65-7.52 (m, 3H), 7.40-7.35 (m, 1H), 3.95 (s, 2H), 3.20 (s, 3H), 2.40 (s, 2H), 1.11 (s, 6H). Two exchangeable protons not observed.

### Example 27: Synthesis of 1-((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)-3-methylazetidin-3-ol (AF-2)

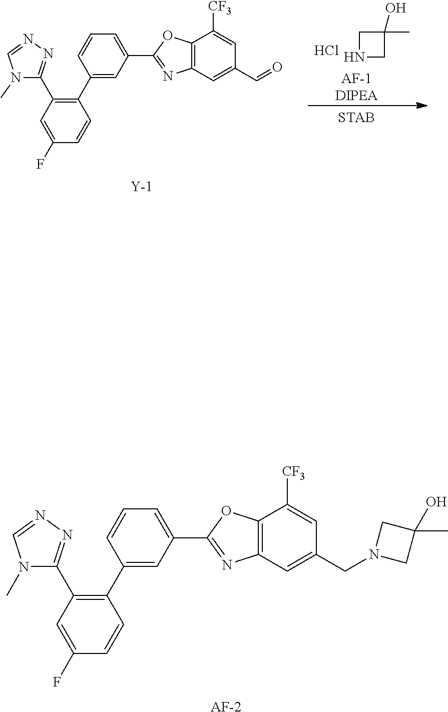

A solution of intermediate (Y-1) (31 mg, 1 Eq, 66 μmol) and 3-methylazetidin-3-ol, HCl (AF-1) (16 mg, 2.0 Eq, 0.13 mmol) in CHCl₃ (1 mL) was stirred 1h at rt, then NaBH(OAc)₃ (42 mg, 3.0 Eq, 0.20 mmol) was added. The reaction mixture was stirred at rt overnight. The reaction mixture was diluted with EtOAc (25 mL) and transferred into a separating funnel. The layer was washed with sat. aq. sol. of NaHCO₃ (2×5 mL) and brine. The combined organic layers were collected, dried (MgSO₄), filtered and concentrated in vacuo. The crude product was purified by chromatography on silica gel (12 g cartridge, 0-25% MeOH/DCM) to afford the title compound (AF-2) (23 mg, 41 μmol, 61%, 95% Purity) as a clear white solid. m/z 538.2 (M+H)⁺ (ES+). ¹H NMR (400 MHz, Methanol-d4) δ 8.41 (s, 1H), 8.23 (dt, J=7.9, 1.3 Hz, 1H), 8.08 (t, J=1.8 Hz, 1H), 7.97 (d, J=1.5 Hz, 1H), 7.81 (dd, J=8.7, 5.5 Hz, 1H), 7.71 (s, 1H), 7.64-7.53 (m, 2H), 7.53-7.43 (m, 2H), 3.89 (s, 2H), 3.41-3.34 (m, 2H), 3.27 (s, 3H), 3.22-3.16 (m, 2H), 1.51 (s, 3H). Exchangeable protons not observed.

### Example 28: Synthesis of 5-((6-Azaspiro[2.5]octan-6-yl)methyl)-2-(4′-fluoro-2′-(4-methyl-4H-1,2,4-triazol-3-yl)-[1,1′-biphenyl]-3-yl)-7-(trifluoromethyl)benzo[d]oxazole (AG-2)

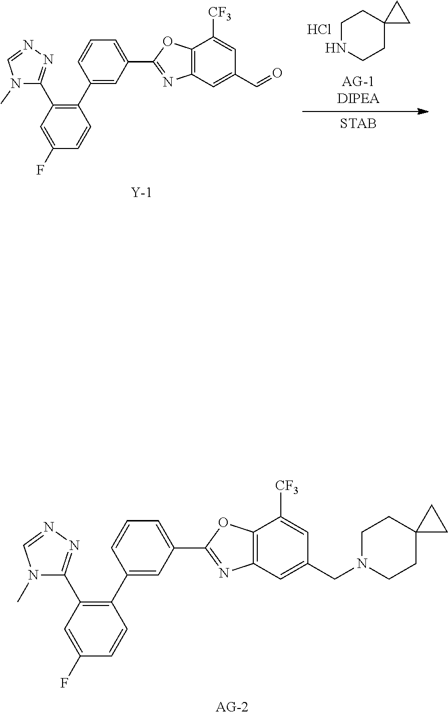

A solution of intermediate (Y-1) (30 mg, 90% Wt, 1 Eq, 58 μmol), 6-azaspiro[2.5]octane, HCl (AG-1) (17 mg, 2.0 Eq, 0.12 mmol) and DIPEA (22 mg, 30 μL, 3.0 Eq, 0.17 mmol) in CHCl₃ (1 mL) was stirred 1 h at rt, then NaBH(OAc)₃ (37 mg, 3.0 Eq, 0.17 mmol) was added. The reaction mixture was stirred at rt overnight. The reaction mixture was diluted with EtOAc (25 mL) and transferred into a separating funnel. The layer was washed with sat. aq. sol. of NaHCO₃ (2×5 mL) and brine. The combined organic layers were collected, dried (MgSO₄), filtered and concentrated in vacuo. The crude product was purified by chromatography on silica gel (12 g cartridge, 0-20% MeOH/DCM) to afford the title compound (AG-2) (20 mg, 35 μmol, 61%, 99% Purity) as a clear white solid. m/z 562.2 (M+H)⁺ (ES+). ¹H NMR (400 MHz, Methanol-d4) δ 8.41 (s, 1H), 8.24 (dt, J=8.0, 1.3 Hz, 1H), 8.09 (t, J=1.7 Hz, 1H), 8.02 (d, J=1.5 Hz, 1H), 7.81 (dd, J=8.6, 5.4 Hz, 1H), 7.77 (d, J=1.8 Hz, 1H), 7.65-7.53 (m, 2H), 7.53-7.44 (m, 2H), 3.78 (s, 2H), 3.28 (s, 3H), 2.59 (s, 4H), 1.49 (s, 4H), 0.34 (s, 4H).

### Example 29: Synthesis of 1-((((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)amino)methyl)cyclopropan-1-ol (AH-2)

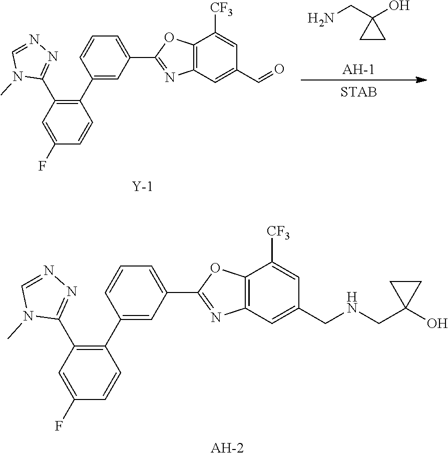

A solution of intermediate (Y-1) (25 mg, 80% Wt, 1 Eq, 43 μmol) and 1-(aminomethyl)cyclopropan-1-ol (AH-1) (7.5 mg, 2.0 Eq. 86 μmol) in CHCl₃ (1 mL) was stirred 1h at rt, then NaBH(OAc)₃ (27 mg, 3.0 Eq, 0.13 mmol) was added. The reaction mixture was stirred at rt overnight. The reaction mixture was diluted with EtOAc (25 mL) and transferred into a separating funnel. The layer was washed with sat. aq. sol. of NaHCO₃ (2×5 mL) and brine. The combined organic layers were collected, dried (MgSO₄), filtered and concentrated in vacuo. The crude product was purified by chromatography on silica gel (12 g cartridge, 0-20% MeOH/DCM) to afford the title compound (AH-2) (5 mg, 9 μmol, 20%, 99% Purity) as a clear white solid. m/z 538.2 (M+H)⁺ (ES+). ¹H NMR (400 MHz, Methanol-d4) δ 8.41 (s, 1H), 8.25 (dt, J=7.9, 1.4 Hz, 1H), 8.17 (d, J=1.6 Hz, 1H), 8.08 (t, J=1.8 Hz, 1H), 7.92 (s, 1H), 7.81 (dd, J=8.6, 5.4 Hz, 1H), 7.66-7.54 (m, 2H), 7.51 (ddd, J=8.8, 3.5, 2.0 Hz, 2H), 4.40 (s, 2H), 3.28 (s, 3H), 3.10 (s, 2H), 0.92-0.85 (m, 2H), 0.76-0.68 (m, 2H). Exchangeable protons not observed.

### Example 30: Synthesis of N-((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)-2-methoxy-2-methylpropan-1-amine (AI-2)

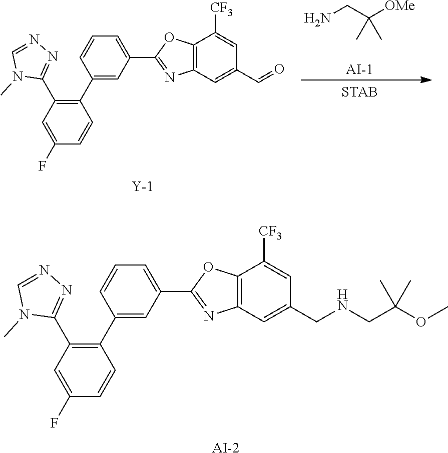

A solution of intermediate (Y-1) (30 mg, 80% Wt, 1 Eq, 51 μmol) and 2-methoxy-2-methylpropan-1-amine (AI-1) (11 mg, 2.0 Eq. 0.1 mmol) in CHCl₃ (1 mL) was stirred 1 h at rt, then NaBH(OAc)₃ (33 mg, 3.0 Eq, 0.15 mmol) was added. The reaction mixture was stirred at rt overnight. The reaction mixture was diluted with EtOAc (25 mL) and transferred into a separating funnel. The layer was washed with sat. aq. sol. of NaHCO₃ (2×5 mL) and brine. The combined organic layers were collected, dried (MgSO₄), filtered and concentrated in vacuo. The crude product was purified by chromatography on silica gel (12 g cartridge, 0-20% MeOH/DCM) to afford the title compound (AI-2) (16 mg, 29 μmol, 56%, 99% Purity) as a clear white solid. m/z 554.2 (M+H)⁺ (ES+). ¹H NMR (400 MHz, Methanol-d4) δ 8.41 (s, 1H), 8.24 (dt, J=8.0, 1.3 Hz, 1H), 8.09 (t, J=1.8 Hz, 1H), 8.03 (s, 1H), 7.84-7.78 (m, 2H), 7.64-7.54 (m, 2H), 7.54-7.43 (m, 2H), 4.01 (s, 2H), 3.27 (s, 3H), 3.20 (s, 3H), 2.60 (s, 2H), 1.23 (s, 6H). Exchangeable proton not observed.

### Example 31: Synthesis of (2-(4′-Fluoro-2′-(4-methyl-4H-1,2,4-triazol-3-yl)-[1,1′-biphenyl]-3-yl)benzo[d]oxazol-5-yl)methanol (AJ-3)

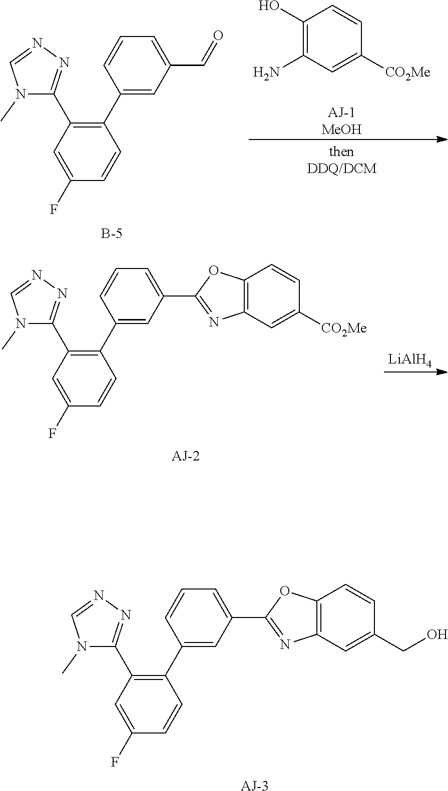

Step 1: Methyl 2-(4′-fluoro-2′-(4-methyl-4H-1,2,4-triazol-3-yl)-[1,1′-biphenyl]-3-yl)benzo[d]oxazole-5-carboxylate (AJ-2). To a solution of intermediate (B-5) (150 mg, 90% Wt, 1 Eq, 480 μmol) in MeOH (2 mL) was added methyl 3-amino-4-hydroxybenzoate (AJ-1) (80.2 mg, 1 Eq, 480 μmol). The resulting mixture was stirred at 45° C. for 12 h. After concentration in vacuo, the residue was dissolved in DCM (2 mL) and 4,5-dichloro-3,6-dioxocyclohexa-1,4-diene-1,2-dicarbonitrile (120 mg, 1.1 Eq, 528 μmol) was then added. After stirring at rt for 1 h, the resulting mixture was diluted with additional DCM (5 mL) and washed sequentially with sat. NaHCO₃ (2×5 mL) and brine (5 mL) and the organic layer dried (Na₂SO₄). The crude product was purified by chromatography on silica gel (40 g cartridge, 0-30% MeOH/DCM) to afford the sub-title compound (AJ-2) (213 mg, 0.42 mmol, 88%, 85% Purity) as a pale brown oil. m/z 429.2 (M+H)⁺ (ES+). ¹H NMR (400 MHz, DMSO-d6) δ 8.43 (s, 1H), 8.35 (d, J=1.6 Hz, 1H), 8.16 (dt, J=8.0, 1.3 Hz, 1H), 8.08 (dd, J=8.6, 1.7 Hz, 1H), 8.01 (t, J=1.8 Hz, 1H), 7.93 (d, J=8.6 Hz, 1H), 7.78 (dd, J=8.6, 5.7 Hz, 1H), 7.60 (td, J=8.6, 2.9 Hz, 1H), 7.56-7.51 (m, 2H), 7.32 (dt, J=7.8, 1.4 Hz, 1H), 3.91 (s, 3H), 3.15 (s, 3H).

Step 2: (2-(4′-Fluoro-2′-(4-methyl-4H-1,2,4-triazol-3-yl)-[1,1′-biphenyl]-3-yl)benzo[d]oxazol-5-yl)methanol (AJ-3). To a solution of the product from step 1 above (AJ-2) (217 mg, 85% Wt, 1 Eq, 431 μmol) in THF (10 mL) was added LiAlH₄ (32.7 mg, 861 μL, 1 molar, 2.0 Eq, 861 μmol) drop-wise at −20° C. This mixture was stirred for 1 h at this temperature. The mixture was quenched with H₂O (0.2 mL), and NaOH (aq. 15%, 0.2 mL), and H₂O (0.6 mL) was added at 0° C. MgSO₄ was added and the mixture was warmed up to rt. The mixture was filtered over a silica plug and the solvent was removed in vacuo. The crude product was purified by chromatography on silica gel (24 g cartridge, 0-30% MeOH/DCM) to afford the title compound (AJ-3) (64 mg, 0.16 mmol, 37%, 99% Purity). m/z 401.2 (M+H)⁺ (ES+). ¹H NMR (400 MHz, Methanol-d4) δ 8.38 (s, 1H), 8.20 (dt, J=8.0, 1.3 Hz, 1H), 8.08 (t, J=1.8 Hz, 1H), 7.78 (dd, J=8.6, 5.5 Hz, 1H), 7.74 (d, J=1.5 Hz, 1H), 7.64 (d, J=8.4 Hz, 1H), 7.54 (td, J=8.3, 3.0 Hz, 2H), 7.50-7.36 (m, 3H), 4.74 (s, 2H), 3.18 (s, 3H). Exchangeable proton not visible.

### Example 32: Synthesis of 1-((((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)amino)methyl)cyclopentan-1-ol, Acetate (AK-2)

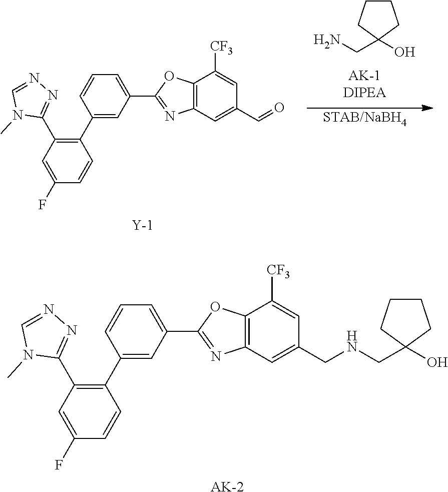

A solution of intermediate (Y-1) (25 mg, 80% Wt, 1 Eq, 43 μmol), 1-(aminomethyl)cyclopentan-1-ol, HCl (AK-1) (13 mg, 2.0 Eq, 43 μmol) and DIPEA (17 mg, 22 μL, 3.0 Eq, 0.13 mmol) in CHCl₃ (1 mL) was stirred for 1 h at rt. NaBH(OAc)₃ (27 mg, 3.0 Eq, 0.13 mmol) was added and the reaction mixture was stirred at rt overnight. An additional 1 Eq. NaBH(OAc)₃ was added and the reaction temperature was raised at 40° C. for 4h. NaBH₄ (1.6 mg, 1 Eq, 43 μmol) was added and the reaction mixture was stirred overnight at 40° C. The mixture was diluted with EtOAc (25 mL) and the organic phase was washed with sat. aq. sol. of NaHCO₃ (10 mL). The organic phase was then washed with HCl 1M solution (20 mL). The organic phase was removed and the aqueous phase basified with a NaOH 2M solution. This aqueous phase was back-extracted with DCM (3×10 mL) and EtOAc (2×10 mL). The combined organic phase was dried (MgSO₄), filtered and concentrated in vacuo. The crude product was purified by chromatography on silica gel (12 g cartridge, 0-20% MeOH/DCM) to afford the title compound (AK-2) (4 mg, 6 μmol, 10%, 99% Purity) as a clear white solid. m/z 566.6 (M+H)⁺ (ES+). ¹H NMR (400 MHz, Methanol-d4) δ 8.38 (s, 1H), 8.21 (dt, J=7.8, 1.4 Hz, 1H), 8.11 (s, 1H), 8.05 (t, J=1.7 Hz, 1H), 7.86 (s, 1H), 7.78 (dd, J=8.6, 5.4 Hz, 1H), 7.69-7.51 (m, 2H), 7.47 (dd, J=8.9, 2.4 Hz, 2H), 4.25 (s, 2H), 3.25 (s, 3H), 2.92 (s, 2H), 1.83 (s, 2H), 1.77-1.59 (m, 6H). Exchangeable proton not visible.

### Example 33: Synthesis of (R)-1-(((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)amino)propan-2-ol (AL-2)

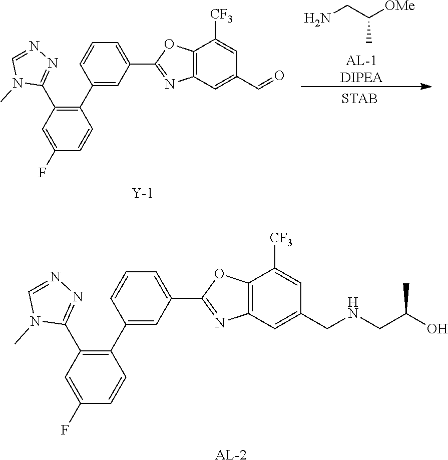

To a solution of (R)-1-aminopropan-2-ol (AL-1) (3.6 mg, 3.8 μL, 1 Eq, 48 μmol) and DIPEA (19 mg, 25 μL, 3.0 Eq, 0.14 mmol) in CHCl₃ (1 mL) was added intermediate (Y-1) (25 mg, 90% Wt, 1 Eq, 48 μmol) and the reaction was stirred for 30 min. NaBH(OAc)₃ (31 mg, 3.0 Eq, 0.14 mmol) was added and the reaction was stirred at rt overnight. The reaction mixture was diluted with EtOAc (10 mL), washed with sat. aq. sol. of NaHCO₃ (2×5 mL) and brine (5 mL). The organic extract was separated, dried (phase separator) and concentrated in vacuo. The crude product was purified by preparative HPLC (Waters, Basic (0.3% ammonium bicarbonate), Basic, Waters X-Bridge Prep-C18, 5 μm, 19×50 mm column, 0-100% MeCN in Water) to afford the title compound (AL-2) (4.00 mg, 7.2 μmol, 15%, 95% Purity) as a white solid. m/z 526.3 (M+H)⁺ (ES+). ¹H NMR (400 MHz, DMSO-d6) δ 8.42 (s, 1H), 8.15-8.11 (m, 1H), 8.07 (s, 1H), 7.92-7.89 (m, 1H), 7.80-7.75 (m, 2H), 7.63-7.53 (m, 3H), 7.40-7.35 (m, 1H), 4.47 (d, J=4.5 Hz, 1H), 3.90 (s, 2H), 3.74-3.66 (m, 1H), 3.22-3.18 (m, 3H), 2.42 (d, J=5.9 Hz, 2H), 1.04 (d, J=6.2 Hz, 3H). One exchangeable proton not observed.

### Example 34: Synthesis of (S)-1-(((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)amino)propan-2-ol (AM-2)

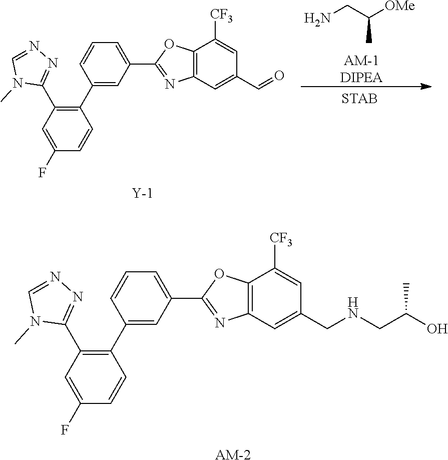

To a solution of (S)-1-aminopropan-2-ol (AM-1) (3.9 mg, 4.1 μL, 1 Eq, 51 μmol) and DIPEA (20 mg, 27 μL, 3.0 Eq, 0.15 mmol) in CHCl₃ (1 mL) was added intermediate (Y-1) (30 mg, 80% Wt, 1 Eq, 51 μmol) and the reaction was stirred for 30 min. NaBH(OAc)₃ (33 mg, 3.0 Eq, 0.15 mmol) was added and the reaction was stirred at rt overnight. The reaction mixture was diluted with EtOAc (10 mL), washed with sat. aq. sol. of NaHCO₃ (2×5 mL) and brine (5 mL). The organic extract was separated, dried (phase separator) and concentrated in vacuo. The crude product was purified by preparative HPLC (Waters, Basic (0.3% ammonium bicarbonate), Basic, Waters X-Bridge Prep-C18, 5 μm, 19×50 mm column, 0-100% MeCN in Water) to afford the title compound (AM-2) (3.43 mg, 6.3 μmol, 12%, 97% Purity) as a white solid. m/z 526.3 (M+H)⁺ (ES+). ¹H NMR (400 MHz, DMSO-d6) δ 8.42 (s, 1H), 8.16-8.10 (m, 1H), 8.07 (s, 1H), 7.92-7.88 (m, 1H), 7.80-7.74 (m, 2H), 7.64-7.53 (m, 3H), 7.40-7.35 (m, 1H), 4.48 (d, J=4.4 Hz, 1H), 3.90 (s, 2H), 3.75-3.65 (m, 1H), 3.24-3.17 (m, 3H), 2.42 (d, J=5.9 Hz, 2H), 1.04 (d, J=6.2 Hz, 3H). One exchangeable proton not observed.

### Example 35: Synthesis of 2-(((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)amino)ethan-1-ol (AN-2)

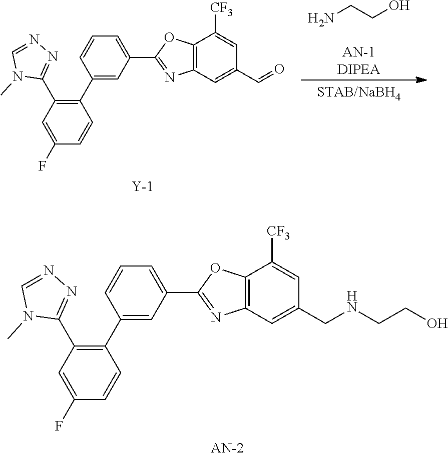

To a solution of 2-aminoethan-1-ol (AN-1) (2.1 mg, 2.1 μL, 1 Eq, 34 μmol) and DIPEA (13 mg, 18 μL, 3.0 Eq, 0.10 mmol) in CHCl₃ (1 mL) was added intermediate (Y-1) (20 mg, 80% Wt, 1 Eq, 34 μmol) and the reaction was stirred for 30 min. NaBH(OAc)₃ (22 mg, 3.0 Eq, 0.10 mmol) was added and the reaction was stirred at rt overnight. Additional 2-aminoethan-1-ol (AN-1) (2.1 mg, 2.1 μL, 1 Eq, 34 μmol) was added and stirred for 2 h. NaBH₄ (3.9 mg, 3.0 Eq, 0.10 mmol) was added and the reaction was stirred at 45° C. overnight. The reaction mixture was diluted with EtOAc (10 mL), washed with sat. aq. sol. of NaHCO₃ (2×5 mL) and brine (5 mL). The organic extract was separated, dried (phase separator) and concentrated in vacuo. The crude product was purified by chromatography on silica gel (4 g cartridge, 0-10% MeOH/DCM) then the column flushed with MeOH. The washings were concentrated in vacuo and triturated with ether to afford the title compound (AN-2) (18 mg, 33 μmol, 97%, 95% Purity) as an off-white solid. m/z 512.4 (M+H)⁺ (ES+). ¹H NMR (400 MHz, Methanol-d4) δ 8.41 (s, 1H), 8.25 (d, J=7.9 Hz, 1H), 8.13 (s, 1H), 8.09 (d, J=1.8 Hz, 1H), 7.87 (s, 1H), 7.84-7.79 (m, 1H), 7.65-7.57 (m, 2H), 7.53-7.47 (m, 2H), 4.25 (s, 2H), 3.80 (t, J=5.5 Hz, 2H), 3.28 (s, 3H), 3.04-2.98 (m, 2H). Two exchangeable protons not observed.

### Example 36: Synthesis of 1-(((2-(4′-Fluoro-2′-(4-methyl-4H-1,2,4-triazol-3-yl)-[1,1′-biphenyl]-3-yl)benzo[d]oxazol-5-yl)methyl)amino)-2-methylpropan-2-ol (AO-2)

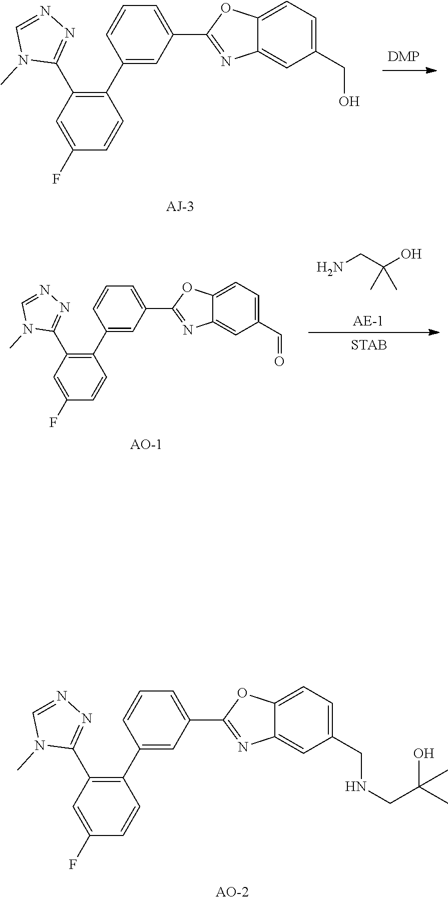

Step 1: 2-(4′-Fluoro-2′-(4-methyl-4H-1,2,4-triazol-3-yl)-[1,1′-biphenyl]-3-yl)benzo[d]oxazole-5-carbaldehyde (AO-1). Compound (AJ-3) (50.0 mg, 1 Eq, 125 μmol) and DMP (106 mg, 2.0 Eq, 250 μmol) were stirred in CHCl₃ (5 mL) at 40° C. for 2 h. The reaction mixture was diluted with EtOAc (20 mL) and washed with sat. aq. NaHCO₃ (10 mL) and brine (10 mL). Organics were separated, dried (MgSO₄), filtered and concentrated in vacuo affording the sub-title compound (AO-1) (56 mg, 125 μmol, 100%, 90% Purity), which was used in the next step without purification. m/z 399.0 (M+H)⁺ (ES+).

Step 2: 1-(((2-(4′-Fluoro-2′-(4-methyl-4H-1,2,4-triazol-3-yl)-[1,1′-biphenyl]-3-yl)benzo[d]oxazol-5-yl)methyl)amino)-2-methylpropan-2-ol (AO-2). A solution of the product from step 1 above (AO-1) (25 mg, 90% Wt, 1 Eq, 56 μmol) and 1-amino-2-methylpropan-2-ol (AE-1) (10 mg, 11 μL, 2.0 Eq, 0.11 mmol) in CHCl₃ (1 mL) was stirred for 1 h at 40° C., then NaBH(OAc)₃ (36 mg, 3.0 Eq, 0.17 mmol) was added. The reaction mixture was stirred at 40° C. overnight. The reaction mixture was diluted with EtOAc (25 mL) and transferred into a separating funnel. The layer was washed with sat. aq. sol. of NaHCO₃ (2×5 mL) and brine. The combined organic layers were collected, dried (MgSO₄), filtered and concentrated in vacuo. The crude product was purified by preparative HPLC (Waters, Basic (0.3% ammonia), Basic, Waters X-Bridge BEH C18 ODB, 5 μm, 30×100 mm column, 20-100% MeCN in Water) to afford the title compound (AO-2) (2 mg, 4 μmol, 7%, 99% Purity). m/z 472.3 (M+H)⁺ (ES+). ¹H NMR (400 MHz, Methanol-d4) δ 8.40 (s, 1H), 8.22 (dt, J=7.9, 1.4 Hz, 1H), 8.10 (t, J=1.8 Hz, 1H), 7.80 (dd, J=8.7, 5.5 Hz, 1H), 7.77 (d, J=1.6 Hz, 1H), 7.66 (d, J=8.3 Hz, 1H), 7.57 (td, J=8.5, 8.1, 2.2 Hz, 2H), 7.48 (ddd, J=8.5, 5.1, 2.2 Hz, 2H), 7.42 (dt, J=8.0, 1.4 Hz, 1H), 3.97 (s, 2H), 3.21 (s, 3H), 2.60 (s, 2H), 1.25 (s, 6H). Exchangeable protons not visible.

### Example 37: Synthesis of 1-(((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)(methyl)amino)-2-methylpropan-2-ol (AP-2)

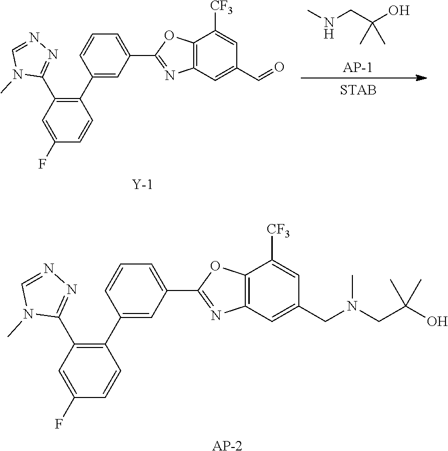

A solution of intermediate (Y-1) (30 mg, 1 Eq, 64 μmol) and 2-methyl-1-(methylamino)propan-2-ol (AP-1) (13 mg, 15 μL, 2.0 Eq, 0.13 mmol) in CHCl₃ (1 mL) was stirred for 1 h at 40° C., then NaBH(OAc)₃ (41 mg, 3.0 Eq, 0.19 mmol) was added. The reaction mixture was stirred at 40° C. overnight. An additional 1 eq. of NaBH(OAc)₃ was added and the mixture was stirred for 3 h at rt. The reaction mixture was diluted with EtOAc (25 mL) and transferred into a separating funnel. The layer was washed with sat. aq. sol. of NaHCO₃ (2×5 mL) and brine. The combined organic layers were collected, dried (MgSO₄), filtered and concentrated in vacuo. The crude product was purified by preparative HPLC (Waters, Basic (0.3% ammonia), Basic, Waters X-Bridge BEH C18 ODB, 5 μm, 30×100 mm column, 40-100% MeCN in Water) to afford the title compound (AP-2) (4 mg, 7 μmol, 10%, 99% Purity) as a red solid. m/z 554.1 (M+H)⁺ (ES+). ¹H NMR (400 MHz, Methanol-d4) δ 8.38 (s, 1H), 8.20 (dt, J=7.9, 1.4 Hz, 1H), 8.05 (t, J=1.8 Hz, 1H), 8.00 (s, 1H), 7.81-7.74 (m, 2H), 7.60-7.51 (m, 2H), 7.50-7.39 (m, 2H), 3.81 (s, 2H), 3.24 (s, 3H), 2.47 (s, 2H), 2.35 (s, 3H), 1.21 (s, 6H). Exchangeable proton not visible.

### Example 38: Synthesis of (S)-2-(4-Methyl-4H-1,2,4-triazol-3-yl)-3′-(5-((3-methylpiperidin-1-yl)methyl)-7-(trifluoromethyl)benzo[d]oxazol-2-yl)-[1,1′-biphenyl]-4-carbonitrile (AQ-6)

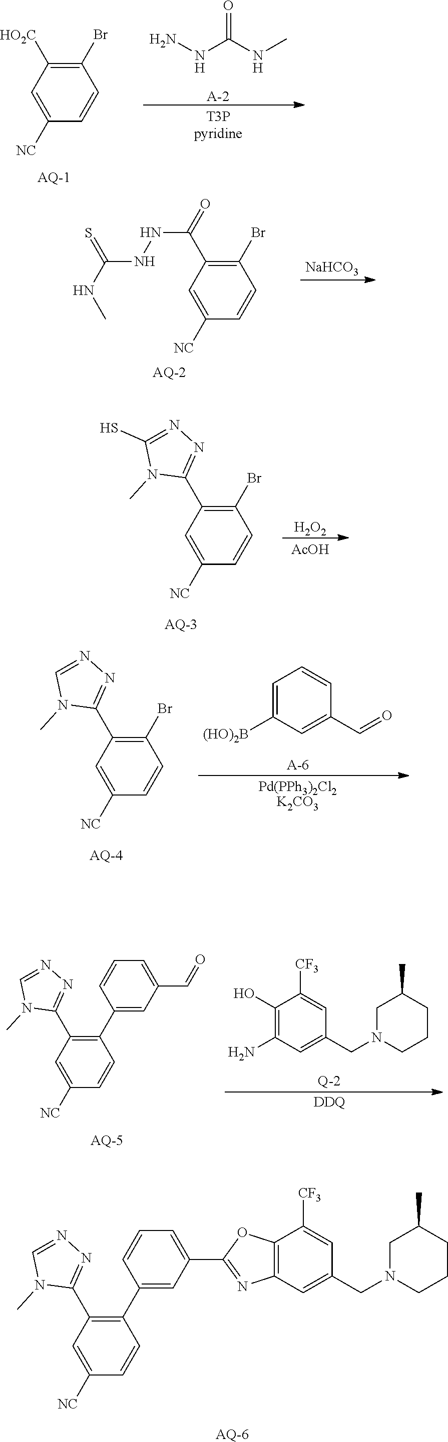

Step 1: 2-(2-Bromo-5-cyanobenzoyl)-N-methylhydrazine-1-carbothioamide (AQ-2). 2-bromo-5-cyanobenzoic acid (AQ-1) (3.71 g, 1 Eq, 16.4 mmol) and pyridine (7.93 mL, 6 Eq, 98.5 mmol) were dissolved in DMF (150 mL) then N-methylhydrazinecarbothioamide (A-2) (5.18 g, 3 Eq, 49.2 mmol) was added followed by T3P (41.8 g, 38.8 mL, 50% Wt, 4 Eq, 65.7 mmol) dropwise. The resulting mixture was then stirred at rt for 16 h. Water (100 mL) was added, stirred for 5 min and the precipitate filtered to afford the sub-title compound (AQ-2) (5.2 g, 16.4 mmol, 100%, 99% Purity) as a white solid. m/z 313/315(M+H)⁺ (ES+).

Step 2: 4-Bromo-3-(4-methyl-5-thioxo-4,5-dihydro-1H-1,2,4-triazol-3-yl)benzonitrile (AQ-3). To a solution of the product from step 1 above (AQ-2) (5.14 g, 1 Eq, 16.4 mmol) in DMF (100 mL) was added aq. NaHCO₃ (100 mL, 1.0 M, 6.1 Eq, 100 mmol) and the reaction mixture was refluxed for 2 h. The reaction mixture was cooled to 0° C. and acidified with 1 M HCl. The precipitate was filtered and the solid washed with EtOAc (100 mL). The solid was dried to afford the sub-title compound (AQ-3) (1.44 g, 4.8 mmol, 29%, 99% Purity). The EtOAc filtrate was washed with brine (50 mL), dried (MgSO₄), filtered and evaporated to afford additional title compound (AQ-3) (1.49 g, 1.7 mmol, 10%, 34% Purity). m/z 295/297 (M+H)⁺ (ES+).

Step 3: 4-Bromo-3-(4-methyl-4H-1,2,4-triazol-3-yl)benzonitrile (AQ-4). To a solution of the product from step 2 above (AQ-3) (2.91 g, 90% Wt, 1 Eq, 8.87 mmol) in DCM (85 mL) and acetic acid (1.07 g, 1.02 mL, 2 Eq, 17.7 mmol) was added hydrogen peroxide (30% in water) (5.03 g, 4.53 mL, 30% Wt, 5 Eq, 44.4 mmol) dropwise with stirring at 0° C. The mixture was warmed up to rt and stirred at this temperature for 2 h. The mixture was basified with sat. aq. sol. of NaHCO₃ to pH 10 and extracted with DCM (3×50 mL). The organic extract was washed with brine (30 mL), dried (MgSO₄) and concentrated to afford the sub-title compound (AQ-4) (1.88 g, 7.1 mmol, 80%, 99% Purity) as a gummy solid. m/z 263.0/265.0 (M+H)⁺ (ES+). ¹H NMR (400 MHz, DMSO-d6) δ 8.68 (s, 1H), 8.11-8.05 (m, 2H), 8.00 (dd, J=8.4, 2.0 Hz, 1H), 3.51 (s, 3H).

Step 4: 3′-Formyl-2-(4-methyl-4H-1,2,4-triazol-3-yl)-[1,1′-biphenyl]-4-carbonitrile (AQ-5). To a solution of (3-formylphenyl)boronic acid (A-6) (442 mg, 1.2 Eq, 2.95 mmol) and the product from step 3 above (AQ-4) (680 mg, 95% Wt, 1 Eq, 2.46 mmol) in degassed dioxane (10 mL) and Water (3 mL) were successively added Pd(PPh₃)₂Cl₂ (345 mg, 0.2 Eq, 491 μmol) and K₂CO₃ (1.02 g, 3 Eq, 7.37 mmol) and stirred at 80° C. for 16 h. The reaction mixture was diluted with EtOAc (75 mL) and washed with sat. aq. sol. of NH₄Cl (2×20 mL), with sat. aq. sol. of NaHCO₃ (2×20 mL) and brine (20 mL). The organics were extracted, dried (MgSO₄) and concentrated in vacuo. The crude product was purified by chromatography on silica gel (80 g cartridge, 0-50% MeOH/DCM) to afford a yellow oil. This oil was dissolved in a small volume of DCM and MTBE was added. A precipitation was observed. The suspension was filtered and the solid was dried to afford the sub-title compound (AQ-5) (320 mg, 1.1 mmol, 43%, 95% Purity) as a pale brown solid. m/z 288.8 (M+H)⁺ (ES+). ¹H NMR (400 MHz, DMSO-d6) δ 9.96 (s, 1H), 8.46 (s, 1H), 8.19 (dd, J=8.1, 1.8 Hz, 1H), 8.14 (d, J=1.7 Hz, 1H), 7.92-7.87 (m, 2H), 7.72 (t, J=1.8 Hz, 1H), 7.57 (t, J=7.6 Hz, 1H), 7.42 (dt, J=7.7, 1.5 Hz, 1H), 3.15 (s, 3H).

Step 5: (S)-2-(4-Methyl-4H-1,2,4-triazol-3-yl)-3′-(5-((3-methylpiperidin-1-yl)methyl)-7-(trifluoromethyl)benzo[d]oxazol-2-yl)-[1,1′-biphenyl]-4-carbonitrile (AQ-6). To a solution of the product from step 4 above (AQ-5) (37 mg, 95% Wt, 1 Eq, 0.12 mmol) in MeOH (1 mL) was added intermediate (Q-2) (53 mg, 80% Wt, 1.2 Eq, 0.15 mmol). The resulting mixture was stirred at 45° C. for 12 h. After concentration in vacuo, the residue was dissolved in DCM (2 mL) and DDQ (30 mg, 1.1 Eq, 0.13 mmol) was then added. After stirring at rt for 1 h, the resulting mixture was diluted with additional DCM (5 mL) and washed sequentially with saturated NaHCO₃ (2×5 mL) and brine (5 mL). The organic layer was dried (Na2SO₄). The reaction mixture was adsorbed onto Celite and concentrated in vacuo. The crude product was purified by chromatography on RP Flash C18 (4 g cartridge, 15-75% MeCN/10 mM ammonium bicarbonate) to afford impure product. The product was loaded onto a column of SCX (500 mg) in MeOH. The column was washed with MeOH and then the product was eluted with 0.7 M ammonia in MeOH. The resultant mixture was concentrated in vacuo to afford impure product. The product was purified by chromatography on silica gel (12 g cartridge, 0-40% MeOH/DCM) to afford the title compound (AQ-6) (9 mg, 0.02 mmol, 10%, 99% Purity) as a pale yellow solid. m/z 557.3 (M+H)⁺ (ES+). ¹H NMR (400 MHz, Methanol-d4) δ 8.44 (s, 1H), 8.29 (dt, J=8.0, 1.3 Hz, 1H), 8.17 (dd, J=8.1, 1.8 Hz, 1H), 8.14-8.09 (m, 2H), 8.02-7.95 (m, 2H), 7.75 (s, 1H), 7.65 (t, J=7.8 Hz, 1H), 7.52 (dt, J=8.1, 1.3 Hz, 1H), 3.72 (s, 2H), 3.29 (s, 3H), 2.87 (dd, J=14.8, 9.5 Hz, 2H), 2.02 (td, J=11.4, 3.1 Hz, 1H), 1.83-1.58 (m, 5H), 1.02-0.85 (m, 4H).

### Example 39: Synthesis of 2-(4′-Fluoro-2′-(4-methyl-4H-1,2,4-triazol-3-yl)-[1,1′-biphenyl]-3-yl)benzo[d]oxazole (AR-2)

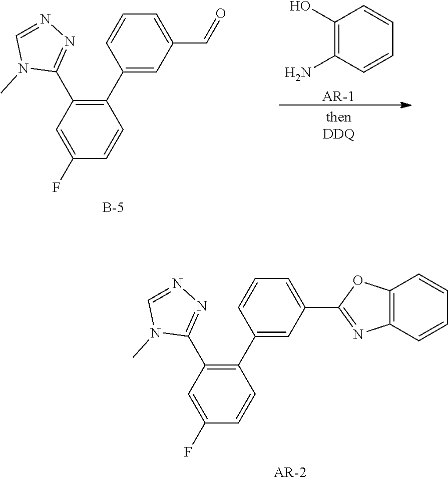

To a solution of intermediate (B-5) (30 mg, 90% Wt, 1 Eq, 96 μmol) in MeOH (2 mL) was added 2-aminophenol (AR-1) (10 mg, 1 Eq, 96 μmol). The resulting mixture was stirred at 45° C. for 12 h. After concentration in vacuo, the residue was dissolved in DCM (2 mL) and DDQ (24 mg, 1.1 Eq, 0.11 mmol) was then added. After stirring at rt for 1 h, the resulting mixture was diluted with additional DCM (5 mL) and washed sequentially with sat. NaHCO₃ (2×5 mL) and brine (5 mL). The organic layer was dried (Na₂SO₄) and the crude product was purified by preparative HPLC (Waters, Basic (0.3% ammonia), Basic, Waters X-Bridge BEH C18 ODB, 5 μm, 30×100 mm column, 25-100% MeCN in Water) to afford the title compound (AR-2) (25 mg, 67 μmol, 70%, 99% Purity) as a pale yellow solid. m/z 371.3 (M+H)⁺ (ES+). ¹H NMR (400 MHz, Methanol-d4) δ 8.41 (s, 1H), 8.27-8.19 (m, 1H), 8.11 (s, 1H), 7.81 (dd, J=8.6, 5.5 Hz, 1H), 7.77 (dd, J=6.4, 2.5 Hz, 1H), 7.71 (dd, J=6.5, 2.3 Hz, 1H), 7.62-7.54 (m, 2H), 7.52-7.41 (m, 4H), 3.21 (s, 3H).

### Example 40: Synthesis of N-ethyl-4-(2-(4-methyl-4H-1,2,4-triazol-3-yl)phenyl)-6-(7-(trifluoromethyl)benzo[d]oxazol-2-yl)pyridin-2-amine (AS-12)

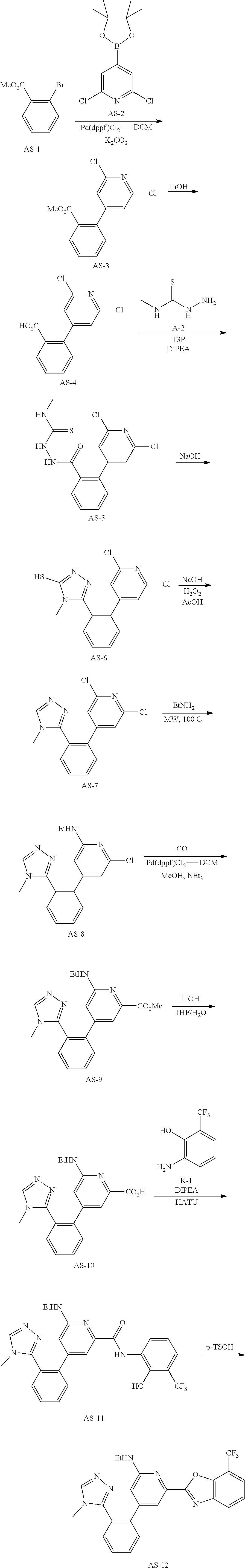

Step 1: Methyl 2-(2,6-dichloropyridin-4-yl)benzoate (AS-3). To a solution of methyl 2-bromobenzoate (AS-1) (765 mg, 0.500 mL, 1 Eq, 3.56 mmol) and 2,6-dichloro-4-(4,4,5,5-tetramethyl-1,3,2-dioxaborolan-2-yl)pyridine (AS-2) (1.34 g, 80% Wt, 1.1 Eq, 3.91 mmol) in 1,4-Dioxane (24 mL)) and water (6 mL) were added Pd(dppf)C12·DCM (145 mg, 0.05 Eq, 178 μmol) and K₂CO₃ (1.47 g, 3 Eq, 10.7 mmol). The reaction mixture was degassed with nitrogen three times and heated overnight at 80° C. The reaction mixture was put through a celite plug, eluting with EtOAc (30 mL). The filtrate was concentrated in vacuo and the crude product was purified by chromatography on silica gel (24 g cartridge, 0-10% MeOH/DCM) to afford the sub-title compound (AS-3) (873.6 mg, 2.9 mmol, 83%, 95% Purity) as a pale yellow solid. ¹H NMR (400 MHz, DMSO-d6) δ 8.00-7.95 (m, 1H), 7.77-7.69 (m, 1H), 7.67-7.61 (m, 1H), 7.56 (s, 2H), 7.50-7.46 (m, 1H), 3.68 (s, 3H).

Step 2: 2-(2,6-Dichloropyridin-4-yl)benzoic acid (AS-4). To a solution of the product from step 1, above (AS-3) (8.38 g, 93% Wt, 1 Eq, 27.6 mmol) in THF (10 mL) was added a solution of LiOH (662 mg, 10 mL, 1 Eq, 27.6 mmol) in water (10 mL) and the mixture was stirred at 40° C. for 16 h. The reaction mixture was concentrated in vacuo. The residue was dissolved in water (30 mL), acidified with 1 M HCl, and extracted with EtOAc (3×50 mL). The organic extracts were combined, dried (MgSO₄) and concentrated in vacuo to afford the sub-title compound (AS-4) (7.34 g, 27 mmol, 97%, 98% Purity) as an off-white solid. m/z 268.1/270.2 (M+H)⁺ (ES+). ¹H NMR (400 MHz, DMSO-d6) δ 13.14 (s, 1H), 7.95 (dd, J=7.7, 1.4 Hz, 1H), 7.76-7.64 (m, 1H), 7.64-7.57 (m, 1H), 7.56 (s, 2H), 7.48-7.42 (m, 1H).

Step 3: 2-(2-(2,6-Dichloropyridin-4-yl)benzoyl)-N-methylhydrazine-1-carbothioamide (AS-5). The product from step 2, above (AS-4) (6.67 g, 1 Eq, 24.9 mmol) and 1-amino-3-methyl-thiourea (A-2) (2.88 g, 1.1 Eq, 27.4 mmol) were dissolved in EtOAc (10 mL). Then DIPEA (7.33 g, 9.88 mL, 2.28 Eq, 56.7 mmol) was added followed by T3P (23.7 g, 22.2 mL, 50% Wt, 1.5 Eq, 37.3 mmol) dropwise. The resulting mixture was then stirred at 70° C. for 16 h. The mixture was diluted with water (20 mL) and the precipitate filtered to afford the sub-title compound (AS-5) (8.84 g, 24.9 mmol, 100%) as a brown solid, which was taken forward without further purification or analysis.

Step 4: 5-(2-(2,6-Dichloropyridin-4-yl)phenyl)-4-methyl-4H-1,2,4-triazole-3-thiol (AS-6). A solution of the product from step 3, above (AS-5) (8.85 g, 1 Eq, 24.9 mmol) in aq. NaOH (2.99 g, 37.3 mL, 2 molar, 3 Eq, 74.7 mmol) was stirred at 50° C. for 16 h. Water (100 mL) was added, the mixture was acidified to pH5 with HCl (aq. 1 M). The precipitate was filtered and concentrated in vacuo to afford the sub-title compound (AS-6) (3.53 g, 10 mmol, 41%, 98% Purity) as a pale brown solid. ¹H NMR (400 MHz, DMSO-d6) δ 13.85 (s, 1H), 7.83-7.68 (m, 4H), 7.44 (s, 2H), 3.26-3.23 (m, 3H).

Step 5: 2,6-Dichloro-4-(2-(4-methyl-4H-1,2,4-triazol-3-yl)phenyl)pyridine (AS-7). To a solution of the product from step 4, above (AS-6) (3.54 g, 98% Wt, 1 Eq, 10.3 mmol) in DCM (90 mL) and acetic acid (18.5 g, 17.7 mL, 30 Eq, 309 mmol) was added hydrogen peroxide (30% in water) (11.7 g, 10.5 mL, 30% Wt, 10 Eq, 103 mmol) dropwise with stirring at 0° C. The mixture was stirred at this temperature for 1 h before being concentrated. The residue was dissolved in water and basified with NaOH (2 M) to pH 10, then organics were separated and the aqueous phase extracted with EtOAc (2×100 mL). The combined organics were washed with brine (50 mL), dried (MgSO4), filtered and concentrated in vacuo to afford the sub-title compound (AS-7) (2.94 g, 9.2 mmol, 89%, 95% Purity) as a pale yellow solid m/z 305.2/307.1 (M+H)⁺ (ES+). ¹H NMR (400 MHz, Methanol-d4) δ 8.54 (s, 1H), 7.85-7.67 (m, 4H), 7.26 (s, 2H), 3.42 (s, 3H).

Step 6: 6-Chloro-N-ethyl-4-(2-(4-methyl-4H-1,2,4-triazol-3-yl)phenyl)pyridin-2-amine (AS-8). The product from step 5 above (AS-7) (330 mg, 1 Eq, 1.08 mmol) and ethylamine (2.10 g, 23.3 mL, 2 molar, 43 Eq, 46.5 mmol) were added to a microwave vial and irradiated for 20 h at 100° C. The reaction mixture was concentrated in vacuo. The crude product was purified by chromatography on silica gel (24 g cartridge, 0-20% MeOH/DCM) to afford the sub-title compound (AS-8) (326 mg, 1.0 mmol, 92%, 96% Purity) as a pale yellow solid. m/z 313.9 (M+H)⁺ (ES+).

Step 7: Methyl 6-(ethylamino)-4-(2-(4-methyl-4H-1,2,4-triazol-3-yl)phenyl)picolinate (AS-9). A glass-lined steel pressure vessel containing a solution of the product from step 6 above (AS-8) (200 mg, 1.00 Eq, 637 μmol) and Pd(dppf)·Cl₂·DCM (52.1 mg, 0.1 Eq, 63.7 μmol) in MeOH (10 mL) was treated with NEt₃ (193 mg, 267 μL, 3.00 Eq, 1.91 mmol). The vessel was sealed, purged with N₂ (×3) followed by CO gas (×2) and pressurizing to 4 bar with CO gas. The mixture was heated overnight at 60° C. The crude was passed through a GF/F filter and volatiles evaporated. The crude product was purified by chromatography on silica gel (24 g cartridge, 0-10% (0.7 M ammonia/MeOH)/DCM) to afford the sub-title compound (AS-9) (119 mg, 0.25 mmol, 39%, 70% Purity) as an orange oil. m/z 338.2 (M+H)⁺ (ES+). ¹H NMR (400 MHz, DMSO-d6) δ 8.46 (s, 1H), 7.73-7.66 (m, 1H), 7.64-7.55 (m, 3H), 6.93 (s, 1H), 6.88 (t, J=5.4 Hz, 1H), 6.33 (s, 1H), 3.78 (s, 3H), 3.21-3.09 (m, 5H), 1.08 (t, J=7.2 Hz, 3H).

Step 8: 6-(Ethylamino)-4-(2-(4-methyl-4H-1,2,4-triazol-3-yl)phenyl)picolinic acid (AS-10). To a solution of the product from step 7 above (AS-9) (119 mg, 1 Eq, 353 μmol) in THF (10 mL) was added a solution of LiOH (25.3 mg, 3 Eq, 1.06 mmol) in water (0.5 mL) and the mixture was stirred at rt for 16 h. The reaction was neutralized with 1 M HCl and extracted with EtOAc (3×10 mL). The aqueous phase was purified by chromatography on RP Flash C18(12 g cartridge, 5-20% (0.1% Formic acid in MeCN)/(0.1% Formic Acid in Water)) to afford the sub-title compound (AS-10) (34 mg, 0.10 mmol, 28%, 95% Purity) as a tan solid. m/z 324.2 (M+H)⁺ (ES+); 322.2 (M−H)⁻ (ES−). ¹H NMR (400 MHz, DMSO-d6) δ 12.70 (br.s, 1H), 8.46 (s, 1H), 7.73-7.67 (m, 1H), 7.65-7.55 (m, 3H), 6.90 (d, J=1.4 Hz, 1H), 6.84-6.79 (m, 1H), 6.32 (s, 1H), 3.22-3.17 (m, 5H), 1.09 (t, J=7.2 Hz, 3H).

Step 9: 6-(Ethylamino)-N-(2-hydroxy-3-(trifluoromethyl)phenyl)-4-(2-(4-methyl-4H-1,2,4-triazol-3-yl)phenyl)picolinamide (AS-11). HATU (52 mg, 1.3 Eq, 0.14 mmol) and DIPEA (34 mg, 46 μL, 2.5 Eq, 0.26 mmol) were added to a solution of the product from step 8 above (AS-10) (34 mg, 1 Eq, 0.11 mmol) in DMF (2 mL). After 20 min, 2-amino-6-(trifluoromethyl)phenol (K-1) (19 mg, 1 Eq, 0.11 mmol) was added and the solution stirred at rt for 16 h. The reaction mixture was quenched using a sat. aq. sol. of NaHCO₃ (50 mL) and extracted with EtOAc (2×30 mL). The organic extracts were combined, dried (phase separator) and concentrated in vacuo to give the crude amide as a brown oil. The crude product (AS-11) was used without further purification.

Step 10: N-ethyl-4-(2-(4-methyl-4H-1,2,4-triazol-3-yl)phenyl)-6-(7-(trifluoromethyl)benzo[d]oxazol-2-yl)pyridin-2-amine (AS-12). To a solution of the product from step 9 above (AS-11) (53 mg, 1 Eq, 0.11 mmol) in toluene (1 mL) was added pTSOH (19 mg, 0.9 Eq, 99 μmol) and the mixture was heated at reflux for 16 h. Additional pTSOH (19 mg, 0.9 Eq, 99 μmol) was added and the reaction was heated at reflux for a further 16 h. The mixture was cooled to rt and quenched with a sat. aq. sol. of NaHCO₃ (10 mL) and extracted with DCM (2×10 mL). The combined organic layers were, dried (MgSO₄), filtered and concentrated in vacuo. The crude product was purified by chromatography on RP Flash C18 (12 g cartridge, 5-65 MeCN/10 mM ammonium bicarbonate) to afford the title compound (AS-12) (8 mg, 0.02 mmol, 20%, 98% Purity) as a tan solid. m/z 465.2 (M+H)⁺ (ES+). ¹H NMR (400 MHz, DMSO-d6) δ 8.45 (s, 1H), 8.16 (d, J=8.0 Hz, 1H), 7.81 (dt, J=7.8, 0.9 Hz, 1H), 7.78-7.58 (m, 5H), 7.22 (d, J=1.3 Hz, 1H), 7.04 (t, J=5.4 Hz, 1H), 6.38 (d, J=1.3 Hz, 1H), 3.29-3.20 (m, 5H), 1.15 (t, J=7.2 Hz, 3H).

### Example 41: Synthesis of 5-(Dimethoxymethyl)-2-(4′-fluoro-2′-(4-methyl-4H-1,2,4-triazol-3-yl)-[1,1′-biphenyl]-3-yl)benzo[d]oxazole (AT-1)

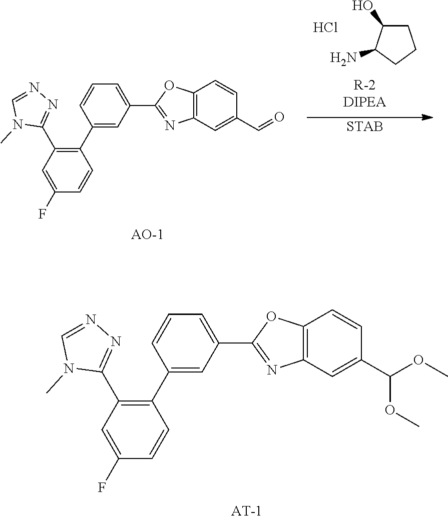

A solution of intermediate (AO-1) (25 mg, 90% Wt, 1 Eq, 56 μmol), (1S,2R)-2-aminocyclopentan-1-ol, HCl (R-2) (16 mg, 2.0 Eq, 0.11 mmol) and DIPEA (22 mg, 30 μL, 3.0 Eq, 0.17 mmol) in CHCl₃ (1 mL) was stirred 1 h at 40° C., then NaBH(OAc)₃ (36 mg, 3.0 Eq, 0.17 mmol) was added. The reaction mixture was stirred at 40° C. overnight. The reaction mixture was diluted with EtOAc (25 mL) and transferred into a separating funnel. The layer was washed with sat. aq. sol. of NaHCO₃ (2×5 mL) and brine. The combined organic layers were collected, dried (MgSO₄), filtered and concentrated in vacuo. The crude product was purified by chromatography on RP Flash C18 (12 g cartridge, 10-80% (0.7 M ammonia/MeOH)/DCM) to afford the title compound (AT-1) (18 mg, 38 μmol, 68%, 95% Purity) as a clear white solid. m/z 445.2 (M+H)⁺ (ES+). ¹H NMR (400 MHz, DMSO-d6) δ 8.42 (s, 1H), 8.13 (dt, J=7.9, 1.4 Hz, 1H), 7.98 (t, J=1.8 Hz, 1H), 7.83-7.74 (m, 3H), 7.60 (td, J=8.5, 2.8 Hz, 1H), 7.57-7.51 (m, 2H), 7.47 (dd, J=8.4, 1.7 Hz, 1H), 7.29 (dt, J=7.9, 1.3 Hz, 1H), 5.52 (s, 1H), 3.28 (s, 6H), 3.14 (s, 3H).

### Example 42: Synthesis of 2-(4-Methyl-4H-1,2,4-triazol-3-yl)-3′-(7-(trifluoromethyl)benzo[d]oxazol-2-yl)-[1,1′-biphenyl]-4-carbonitrile (AU-1)

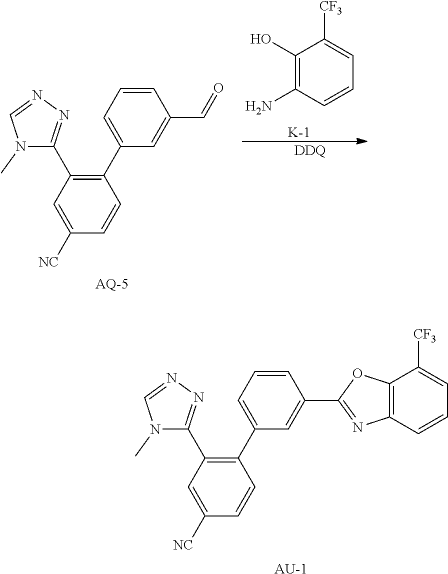

To a solution of intermediate (AQ-5) (30 mg, 95% Wt, 1 Eq, 99 μmol) in MeOH (1 mL) was added 2-amino-6-(trifluoromethyl)phenol (K-1) (19 mg, 1.1 Eq, 0.11 mmol). The resulting mixture was stirred at 45° C. for 12 h. After concentration in vacuo, the residue was dissolved in DCM (2 mL) and DDQ (25 mg, 1.1 Eq, 0.11 mmol) was then added. After stirring at rt for 1 h, the resulting mixture was diluted with EtOAc (25 mL) and washed sequentially with sat. NaHCO₃ (2×5 mL) and brine (5 mL) solutions. The organic layer was dried (Na₂SO₄), filtered and adsorbed onto Celite and concentrated in vacuo. The crude product was purified by chromatography on RP Flash C18 (4 g cartridge, 15-75% (0.1% Formic acid in MeCN)/(0.1% Formic Acid in Water)) to afford the title compound (AU-1) (23 mg, 51 μmol, 51%, 98% Purity) as a dark red solid. m/z 446.2 (M+H)⁺ (ES+). ¹H NMR (400 MHz, Methanol-d4) δ 8.45 (s, 1H), 8.33-8.27 (m, 1H), 8.17 (dd, J=8.0, 1.7 Hz, 1H), 8.15-8.09 (m, 2H), 8.04 (d, J=8.0 Hz, 1H), 7.98 (d, J=8.1 Hz, 1H), 7.75 (d, J=7.7 Hz, 1H), 7.63 (dt, J=20.8, 7.8 Hz, 2H), 7.52 (d, J=7.6 Hz, 1H), 3.29 (s, 3H).

### Example 43: Synthesis of (R)-6-Bromo-2-(3-(1-(4-methyl-4H-1,2,4-triazol-3-yl)propan-2-yl)phenyl)-7-(trifluoromethyl)-1H-benzo[d]imidazole (AV-9) and (S)-6-Bromo-2-(3-(1-(4-methyl-4H-1,2,4-triazol-3-yl)propan-2-yl)phenyl)-7-(trifluoromethyl)-1H-benzo[d]imidazole (AV-10)

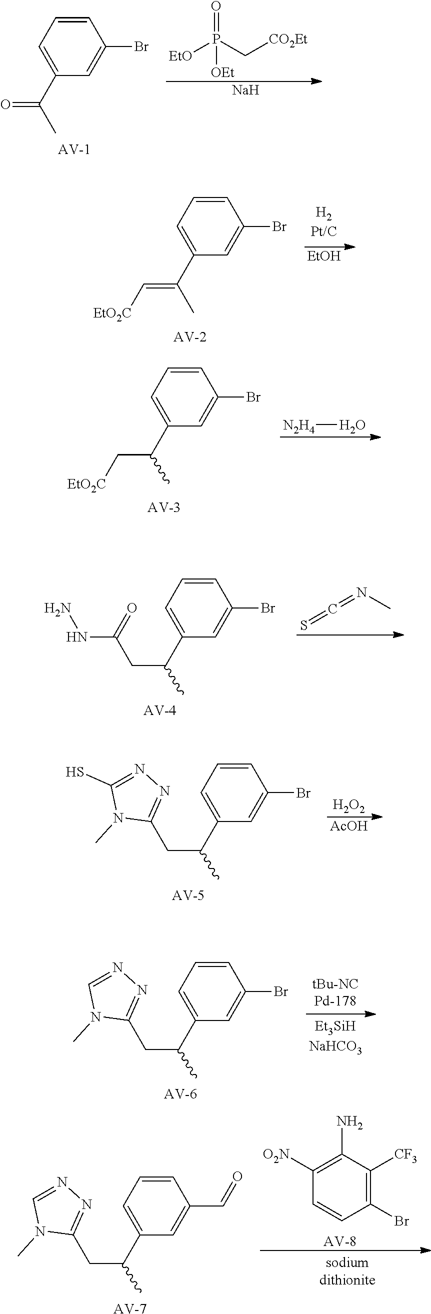

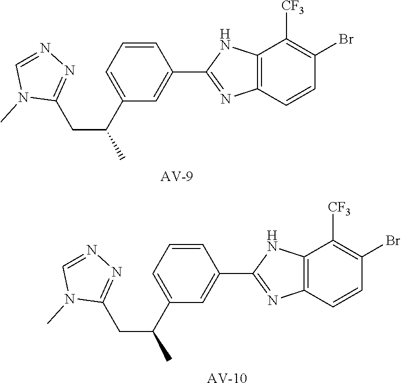

Step 1: 2-Ethyl 3-(3-bromophenyl)but-2-enoate (AV-2). To a cooled (ice bath) suspension of NaH (482 mg, 60% Wt, 1.2 Eq, 12.1 mmol) in THF (50 mL) was added dropwise ethyl 2-(diethoxyphosphoryl)acetate (2.70 g, 2.39 mL, 1.2 Eq, 12.1 mmol). After stirring for 20 min at rt, 1-(3-bromophenyl)ethan-1-one (AV-1) (2.00 g, 1.34 mL, 1 Eq, 10.0 mmol) was added dropwise. The suspension was stirred for further 16 h. Solvents were evaporated and the residue partitioned between EtOAc (50 mL) and water (50 mL). The organic phase was washed with water (20 mL), dried (MgSO₄), filtered and evaporated to afford the sub-title compound (AV-2) (2.75 g), as a 85:15 mixture of geometric isomers. m/z 269/271 (M+H)⁺ (ES+). ¹H NMR (400 MHz, DMSO-d6) δ 7.76 (t, J=1.9 Hz, 1H), 7.60 (tdd, J=8.0, 2.0, 1.0 Hz, 2H), 7.38 (t, J=7.9 Hz, 1H), 6.19 (q, J=1.3 Hz, 1H), 4.16 (q, J=7.1 Hz, 2H), 2.50 (d, J=1.4 Hz, 3H), 1.25 (t, J=7.1 Hz, 3H).

Step 2: (+/−) Ethyl 3-(3-bromophenyl)butanoate (AV-3). To a solution of the products from step 1, above (AV-2) (2.40 g, 1 Eq, 8.92 mmol) in EtOH (40 mL), 5% Pt/C in water (B103032-5) (240 mg, 1 Eq, 8.92 mmol) was added and stirred under 1 atm of H₂ for 4 h. The reaction mixture was filtered and evaporated and the crude residue purified by chromatography on silica gel (80 g cartridge, 0-10% acetone/isohexanes) to afford the sub-title compound (AV-3) (293 mg, 85% Purity) as a colourless oil and some mix fractions. The mix fractions were purified by chromatography on silica gel (40 g (Gold column) cartridge, 0-5% EtOAc/isohexane) to afford additional sub-title compound (AV-3) (981 mg, 73% Purity) as a colourless oil. m/z 271/273 (M+H)⁺ (ES+). ¹H NMR (400 MHz, DMSO-d6) δ 7.46 (t, J=1.8 Hz, 1H), 7.38 (dt, J=7.0, 1.9 Hz, 1H), 7.29-7.22 (m, 2H), 3.98 (q, J=7.1 Hz, 2H), 3.21-3.11 (m, 1H), 2.62-2.55 (m, 2H), 1.21 (dd, J=7.0, 1.5 Hz, 3H), 1.09 (t, J=7.1 Hz, 3H).

Step 3: (+/−) 3-(3-Bromophenyl)butanehydrazide (AV-4). To a solution of the product from step 2, above (AV-3) (1.27 g, 73% Wt, 1 Eq, 3.42 mmol) in EtOH (15 mL) was added hydrazine (24-25% in water) (329 mg, 322 μL, 3 Eq, 10.3 mmol) dropwise at 0° C. The mixture was stirred at rt for 16 h. Volatiles were evaporated and hydrazine hydrate, 100% (hydrazine, 64%) (5 g, 5 mL, 64% Wt, 3e+1 Eq, 0.1 mol) added. The reaction mixture was warmed up to 80° C. and stirred for 2 h. The reaction was warmed up to 100° C. and stirred for further 2 h. Volatiles were evaporated, azeotroping with toluene. The obtained oil was poured in iced water and solvents were evaporated in vacuo to afford the sub-title compound (AV-4) (596 mg, 1.7 mmol, 50%, 74% Purity) as a colourless oil. m/z 257/259 (M+H)⁺ (ES+). ¹H NMR (400 MHz, DMSO-d6) δ 8.94 (s, 1H), 7.47-7.31 (m, 1H), 7.32-7.11 (m, 3H), 4.18 (s, 2H), 3.22-3.08 (m, 1H), 2.36-2.18 (m, 2H), 1.16 (d, J=7.0 Hz, 3H).

Step 4: (+/−) 5-(2-(3-Bromophenyl)propyl)-4-methyl-4H-1,2,4-triazole-3-thiol (AV-5). To a solution of the product from step 3, above (AV-4) (4.99 g, 1 Eq, 19.4 mmol) in THF (50 mL) was added isothiocyanatomethane (1.42 g, 1.33 mL, 1 Eq, 19.4 mmol). The mixture was stirred at rt for 16 h. The mixture was concentrated, and the residue treated with 2 M NaOH (1.55 g, 19.4 mL, 2 molar, 2 Eq, 38.8 mmol) and stirred at rt for 2 h. The mixture was acidified using 1 M HCl to pH 3 and extracted with EtOAc (3×5 mL). Organics were combined, dried (MgSO₄), filtered and evaporated to afford the sub-title compound (AV-5) (5.14 g, 16.5 mmol, 84.9%) as a white gum. m/z 312/314 (M+H)⁺ (ES+).

Step 5: (+/−) 3-(2-(3-Bromophenyl)propyl)-4-methyl-4H-1,2,4-triazole (AV-6). To a solution of the product from step 4, above (AV-5) (5.14 g, 1 Eq, 16.5 mmol) in DCM (54 mL) and AcOH (27 mL) was added hydrogen peroxide (30% in water) (18.7 g, 16.8 mL, 30% Wt, 10 Eq, 165 mmol) dropwise with stirring at 0° C. The mixture was stirred at this temperature for 1 h before being concentrated. The residue was dissolved in water and basified with 2 M NaOH to pH 10, then organics were separated and aqueous phase extracted with EtOAc (2×100 mL) and organics washed with brine (100 mL). Organics were combined, dried (MgSO₄), filtered and evaporated. The crude product was purified by chromatography on silica gel (120 g cartridge, 0-10% MeOH/DCM) to afford the sub-title compound (AV-6) (2.59 g, 8.6 mmol, 52%, 93% Purity) as a brown oil. m/z 280/282 (M+H)⁺ (ES+). ¹H NMR (400 MHz, DMSO-d6) δ 8.28 (s, 1H), 7.48 (t, J=1.8 Hz, 1H), 7.38 (dt, J=7.4, 1.8 Hz, 1H), 7.30-7.20 (m, 2H), 3.47 (s, 3H), 3.30-3.21 (m, 1H), 3.00-2.94 (m, 2H), 1.26 (d, J=7.0 Hz, 3H).

Step 6: (+/−) 3-(1-(4-Methyl-4H-1,2,4-triazol-3-yl)propan-2-yl)benzaldehyde (AV-7). The product from step 5, above (AV-6) (190 mg, 62% Wt, 1 Eq, 420 μmol), Pd-178 (10.0 mg, 0.05 Eq, 21.0 μmol), triethylsilane (147 mg, 201 μL, 3 Eq, 1.26 mmol), tert-butyl(methylidyne)-14-azane (42.5 mg, 57.8 μL, 1.2 Eq, 505 mol), and NaHCO₃ (44.6 mg, 1 Eq, 420 μmol) in DMF (2 mL) were added to a MW vial and stirred at 65° C. for 8 h under nitrogen. The mixture was diluted with water (10 mL) and extracted with TBME (3×10 mL). Organics were combined, dried (MgSO₄), filtered and concentrated under vacuum. The crude product was purified by chromatography on RP Flash C18 (12 g cartridge, 5-40% MeCN/10 mM ammonium bicarbonate) to afford the sub-title compound (AV-7) (5 mg) as a white solid. The extraction waters were further extracted with EtOAc (3×20 mL), organics combined, dried (MgSO₄), filtered and concentrated in vacuo. The crude product was purified by chromatography on RP Flash C18 (12 g cartridge, 5-40% MeCN/10 mM ammonium bicarbonate) to afford additional sub-title compound (AV-7) (7 mg) as a white solid. Both solids were combined to afford the sub-title compound (AV-7) (12 mg, 52 μmol, 12%, 99% Purity) as a white solid. ¹H NMR (400 MHz, Chloroform-d) δ 9.98 (s, 1H), 7.95 (s, 1H), 7.75-7.71 (m, 2H), 7.46-7.42 (m, 2H), 3.56-3.46 (m, 1H), 3.30 (s, 3H), 3.08 (dd, J=15.0, 7.3 Hz, 1H), 2.94 (dd, J=15.0, 7.3 Hz, 1H), 1.45 (d, J=7.0 Hz, 3H).

Step 7: (R)-6-Bromo-2-(3-(1-(4-methyl-4H-1,2,4-triazol-3-yl)propan-2-yl)phenyl)-7-(trifluoromethyl)-1H-benzo[d]imidazole (AV-9) and (S)-6-Bromo-2-(3-(1-(4-methyl-4H-1,2,4-triazol-3-yl)propan-2-yl)phenyl)-7-(trifluoromethyl)-1H-benzo[d]imidazole (AV-10). To a stirred solution of the product from step 6, above (AV-7) (12 mg, 1 Eq, 52 μmol) in MeOH (2 mL) was added a solution of 1M sodium dithionite (91 mg, 0.52 mL, 1 molar, 10 Eq, 0.52 mmol) and 3-bromo-6-nitro-2-(trifluoromethyl)aniline (AV-8) (22 mg, 1.5 Eq, 79 μmol) at rt. The resulting mixture was stirred for 1 h at 65° C. The mixture was cooled to rt, diluted with water (10 mL) and extracted with EtOAc (2×10 mL). the combined organic layers were washed with brine, dried (MgSO₄), filtered and concentrated in vacuo. The crude was purified by chiral SFC (Waters, Basic (0.1% ammonia), Basic, Waters IH 10×250 mm, 5 μm, 25% EtOH (0.1% ammonia), 75% CO₂ to afford: The title compound (R) enantiomer (AV-9) (6.6 mg, 13 μmol, 26%, 94% Purity). m/z 464/466 (M+H)⁺ (ES+). ¹H NMR (400 MHz, DMSO-d6) δ 8.28 (s, 1H), 8.13 (s, 1H), 8.06 (dt, J=7.5, 1.6 Hz, 1H), 7.76 (d, J=8.5 Hz, 1H), 7.61 (d, J=8.5 Hz, 1H), 7.51-7.42 (m, 2H), 3.47 (s, 3H), 3.42-3.34 (m, 2H), 3.05 (d, J=7.4 Hz, 2H), 1.35 (d, J=7.0 Hz, 3H). The title compound (S) enantiomer (AV-10) (6.2 mg, 13 μmol, 25%, 99% Purity). m/z 464/466 (M+H)⁺ (ES+). ¹H NMR (400 MHz, DMSO-d6) δ 8.28 (s, 1H), 8.13 (s, 1H), 8.06 (dt, J=7.5, 1.6 Hz, 1H), 7.76 (d, J=8.5 Hz, 1H), 7.61 (d, J=8.5 Hz, 1H), 7.51-7.42 (m, 2H), 3.47 (s, 3H), 3.38 (q, J=7.2 Hz, 1H), 3.05 (d, J=7.4 Hz, 2H), 1.35 (d, J=6.9 Hz, 3H).

### Example 44: Synthesis of N,N-dimethyl-2-((2-(3-(1-(4-methyl-4H-1,2,4-triazol-3-yl)propan-2-yl)phenyl)-7-(trifluoromethyl)-1H-benzo[d]imidazol-6-yl)oxy)ethan-1-amine (AW-4)

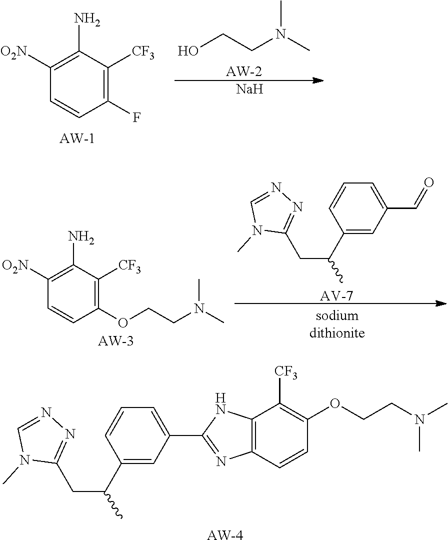

Step 1: 3-(2-(Dimethylamino)ethoxy)-6-nitro-2-(trifluoromethyl)aniline (AW-3). To a solution of 2-(dimethylamino)ethan-1-ol (AW-2) (29.8 mg, 33.7 μL, 1.5 Eq, 335 μmol) in THF (2 mL) was added NaH (14.3 mg, 60% Wt, 1.6 Eq, 357 μmol) at rt under N₂. A solution of 3-fluoro-6-nitro-2-(trifluoromethyl)aniline (AW-1) (50.0 mg, 1 Eq, 223 μmol) in THF (1 mL) was added and the mixture was stirred at rt overnight. The reaction was partitioned between EtOAc (10 mL) and water (10 mL). The organic layer was washed with water (10 mL), dried (MgSO4) and concentrated in vacuo to afford the sub-title compound (AW-3) (62 mg, 0.20 mmol, 90%, 95% Purity) as a brown solid. m/z 294.1 (M+H)⁺ (ES+). ¹H NMR (400 MHz, DMSO-d6) δ 8.32 (d, J=9.7 Hz, 1H), 7.53 (bs, 2H), 6.65 (d, J=9.3 Hz, 1H), 4.26 (t, J=5.6 Hz, 2H), 2.64 (t, J=5.6 Hz, 2H), 2.21 (s, 6H).

Step 2: N,N-dimethyl-2-((2-(3-(1-(4-methyl-4H-1,2,4-triazol-3-yl)propan-2-yl)phenyl)-7-(trifluoromethyl)-1H-benzo[d]imidazol-6-yl)oxy)ethan-1-amine (AW-4). To a stirred solution of intermediate (AV-7) (48 mg, 1 Eq, 0.21 mmol) in MeOH (10 mL) was added a solution of 1M sodium dithionite (0.37 g, 2.1 mL, 1 molar, 10 Eq, 2.1 mmol) and the product from step 1 above (AW-3) (62 mg, 1 Eq, 0.21 mmol) at rt. The resulting mixture was stirred for 1 h at 65° C. The mixture was cooled to rt, diluted with water (40 mL) and extracted with EtOAc (2×40 mL). The organic extracts were washed with brine (20 mL), dried (MgSO₄), filtered and concentrated in vacuo. The crude compound was purified by prep HPLC to afford the title compound (AW-4) (6 mg, 0.01 mmol, 6%, 99% Purity) as a white solid. m/z 473.3 (M+H)⁺ (ES+). ¹H NMR (400 MHz, DMSO-d6) δ 8.28 (s, 1H), 8.11 (s, 1H), 8.04 (d, J=7.5 Hz, 1H), 7.78 (s, 1H), 7.45 (t, J=7.6 Hz, 1H), 7.39 (d, J=7.6 Hz, 1H), 7.18 (d, J=8.9 Hz, 1H), 4.20 (t, J=5.9 Hz, 2H), 3.47 (s, 3H), 3.42-3.33 (m, 1H), 3.05 (d, J=7.4 Hz, 2H), 2.67 (t, J=5.9 Hz, 2H), 2.24 (s, 6H), 1.35 (d, J=6.9 Hz, 3H). Exchangeable NH not observed.

### Example 45: Synthesis of 2-(3-(1-(4-Methyl-4H-1,2,4-triazol-3-yl)propan-2-yl)phenyl)-6-(2-(pyrrolidin-1-yl)ethoxy)-7-(trifluoromethyl)-1H-benzo[d]imidazole (AX-3)

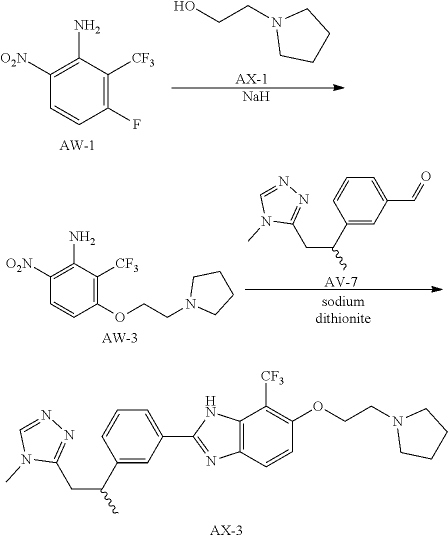

Step 1: 6-Nitro-3-(2-(pyrrolidin-1-yl)ethoxy)-2-(trifluoromethyl)aniline (AX-2). To a solution of 2-(pyrrolidin-1-yl)ethan-1-ol (AX-1) (77.1 mg, 0.09 mL, 1.5 Eq, 669 μmol) in THF (2 mL) was added NaH (28.6 mg, 60% Wt, 1.6 Eq, 714 μmol) at rt under nitrogen. A solution of 3-fluoro-6-nitro-2-(trifluoromethyl)aniline (AW-1) (100 mg, 1 Eq, 446 μmol) in THF (1 mL) was added and the mixture was stirred at rt overnight. The reaction was partitioned between EtOAc (10 mL) and water (10 mL). The organic layer was washed with water (10 mL), dried (MgSO₄) and concentrated in vacuo to afford the sub-title compound (AX-2) (144 mg, 0.43 mmol, 96%, 95% Purity) as a yellow oil. m/z 320.2 (M+H)⁺ (ES+). ¹H NMR (400 MHz, DMSO-d6) δ 8.32 (d, J=9.7 Hz, 1H), 7.53 (s, 2H), 6.65 (d, J=9.7 Hz, 1H), 4.28 (t, J=5.8 Hz, 2H), 2.80 (t, J=5.8 Hz, 2H), 1.73-1.58 (m, 4H). 4H under water.

Step 2: 2-(3-(1-(4-Methyl-4H-1,2,4-triazol-3-yl)propan-2-yl)phenyl)-6-(2-(pyrrolidin-1-yl)ethoxy)-7-(trifluoromethyl)-1H-benzo[d]imidazole (AX-3). To a stirred solution of intermediate (AV-7) (45 mg, 1 Eq, 0.19 mmol) in MeOH (10 mL) was added a solution of 1 M sodium dithionite (0.34 g, 1.9 mL, 1 molar, 10 Eq, 1.9 mmol) and the product from step 1 above (AX-2) (62 mg, 1 Eq, 0.19 mmol) at rt. The resulting mixture was stirred for 1 h at 65° C. and at rt for 16 h. The mixture was cooled to rt, diluted with water (40 mL) and extracted with EtOAc (2×40 mL). The combined organic layers were washed with brine, dried (MgSO₄), filtered and concentrated in vacuo. Water fraction was evaporated, 10% MeOH/DCM was added, filtered, and evaporated. The crude product was purified by preparative HPLC (Waters, Basic (0.1% ammonium bicarbonate), Basic Waters XBridge BEH C18 ODB, 5 μm, 30×100 mm column 0-100% MeCN in water, to afford the title compound (AX-3) (4.1 mg, 8.1 μmol, 4.2%, 99% Purity) as a tan solid. m/z 499.2 (M+H)⁺ (ES+). ¹H NMR (400 MHz, DMSO-d6) δ 8.27 (s, 1H), 8.12 (s, 1H), 8.04 (d, J=7.7 Hz, 1H), 7.81-7.71 (m, 1H), 7.44 (t, J=7.7 Hz, 1H), 7.37 (d, J=7.4 Hz, 1H), 7.14 (d, J=8.9 Hz, 1H), 4.21 (t, J=6.1 Hz, 2H), 3.46 (s, 3H), 3.40-3.35 (m, 1H), 3.05 (d, J=7.4 Hz, 2H), 2.83 (t, J=6.0 Hz, 2H), 2.57-2.53 (m, 4H), 1.72-1.66 (m, 4H), 1.34 (d, J=7.0 Hz, 3H). One exchangeable proton not observed.

### Example 46: Synthesis of (2-(3-(1-(4-Methyl-4H-1,2,4-triazol-3-yl)propan-2-yl)phenyl)-7-(trifluoromethyl)-1H-benzo[d]imidazol-6-yl)(4-methylpiperazin-1-yl)methanone (AY-5)

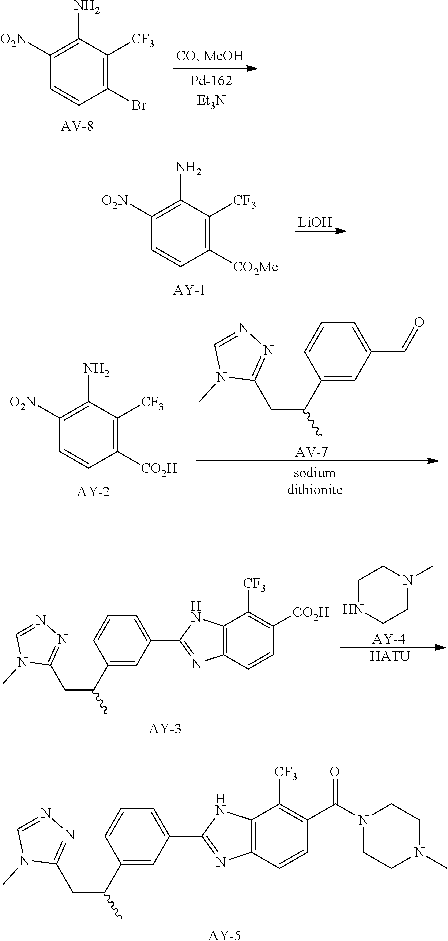

Step 1: Methyl 3-amino-4-nitro-2-(trifluoromethyl)benzoate (AY-1). A glass-lined steel pressure vessel containing a solution of 3-bromo-6-nitro-2-(trifluoromethyl)aniline (AV-8) (300 mg, 1.00 Eq, 1.05 mmol) and Pd-162 (21.0 mg, 0.05 Eq, 52.6 μmol) in MeOH (6 mL) was treated with Et₃N (320 mg, 440 μL, 3.00 Eq, 3.16 mmol). The vessel was sealed, then purged with N₂ (×3) before purging with CO gas (×2) and finally pressurising to -4 bar with CO gas. The mixture was heated overnight at 65° C. The crude was passed through a GF/F filter and solvents were removed in vacuo to afford the sub-title compound (AY-1) (272 mg, 0.82 mmol, 78%, 80% Purity) as a brown oil. m/z 265.1 (M+H)⁺ (ES+). ¹H NMR (400 MHz, DMSO-d6) δ 8.36 (d, J=8.7 Hz, 1H), 7.50 (bs, 2H), 6.83 (dd, J=8.7, 0.7 Hz, 1H), 3.86 (s, 3H).

Step 2: 3-Amino-4-nitro-2-(trifluoromethyl)benzoic acid (AY-2). To a solution of the product from step 1 above (AY-1) (272 mg, 80% Wt, 1 Eq, 824 μmol) in MeOH (10 mL), a solution of 1 M LiOH (49.3 mg, 2.06 mL, 1 molar, 2.5 Eq, 2.06 mmol) was added at rt and stirred at 45° C. for 16 h. EtOAc (10 mL) and water (20 mL) was added, phases separated and the aqueous washed with EtOAc (2×10 mL). The aqueous phase was acidified with 1 M HCl (10 mL) and extracted with EtOAc (2×10 mL). Organics were combined, dried (MgSO₄), filtered and evaporated to afford the sub-title compound (AY-2) (122 mg, 0.46 mmol, 56%, 95% Purity) as a yellow solid. m/z 249.1 (M−H)⁻ (ES−). ¹H NMR (400 MHz, DMSO-d6) δ 13.99 (s, 1H), 8.33 (d, J=8.7 Hz, 1H), 7.48 (s, 2H), 6.77 (d, J=8.7 Hz, 1H).

Step 3: 2-(3-(1-(4-Methyl-4H-1,2,4-triazol-3-yl)propan-2-yl)phenyl)-7-(trifluoromethyl)-1H-benzo[d]imidazole-6-carboxylic acid (AY-3). To a stirred solution of intermediate (AV-7) (183 mg, 1 Eq, 800 μmol) in MeOH (20 mL) was added a solution of 1M sodium dithionite (1.39 g, 8.00 mL, 1 molar, 10 Eq, 8.00 mmol) and the product from step 2 above (AY-2) (200 mg, 1 Eq, 800 μmol) at rt and stirred for 1 h at 65° C. The mixture was cooled to rt, diluted with water (40 mL) and extracted with EtOAc (2×40 mL). The combined organic layers were washed with brine, dried (MgSO₄), filtered and concentrated in vacuo to afford the sub-title compound (AY-3) (113 mg, 0.25 mmol, 31%, 95% Purity) as a yellow solid. m/z 430.1 (M+H)⁺ (ES+). ¹H NMR (400 MHz, DMSO-d6) δ 13.50 (s, 1H), 8.29 (s, 1H), 8.15-8.03 (m, 2H), 7.85 (d, J=8.3 Hz, 1H), 7.52-7.42 (m, 3H), 3.49 (s, 3H), 3.44-3.35 (m, 2H), 3.07 (d, J=7.4 Hz, 2H), 1.36 (d, J=6.9 Hz, 3H).

Step 4: (2-(3-(1-(4-Methyl-4H-1,2,4-triazol-3-yl)propan-2-yl)phenyl)-7-(trifluoromethyl)-1H-benzo[d]imidazol-6-yl)(4-methylpiperazin-1-yl)methanone (AY-5). To a solution of 1-methylpiperazine (AY-4) (52.3 mg, 58.0 μL, 1.2 Eq, 523 μmol) and the product from step 3 above (AY-3) (187 mg, 1 Eq, 435 μmol) in DMF (5 mL) at rt was added DIPEA (169 mg, 228 μL, 3 Eq, 1.31 mmol) and HATU (199 mg, 1.2 Eq, 523 μmol). The reaction was stirred at rt for 18 h. Additional HATU (199 mg, 1.2 Eq, 523 μmol) and DIPEA (169 mg, 228 μL, 3 Eq, 1.31 mmol) were added and the reaction mixture stirred for further 6 h at rt. The reaction was diluted with DCM (10 mL) and brine (5 mL). The layers were separated and the aqueous extracted with DCM (3×10 mL). The combined organic layers were combined, dried (MgSO₄), filtered and concentrated in vacuo. The crude product was purified by chromatography on silica gel (12 g cartridge, 0-10% (0.7 M ammonia/MeOH)/DCM) to afford the title compound (AY-5) (155 mg, 0.30 mmol, 69%, 99% Purity) as a brown glass. m/z 512.3 (M+H)⁺ (ES+). ¹HNMR (400 MHz, DMSO-d6) δ 8.28 (s, 1H), 8.12 (s, 1H), 8.07 (d, J=7.6 Hz, 1H), 7.87 (d, J=8.1 Hz, 1H), 7.51-7.42 (m, 2H), 7.13 (d, J=8.2 Hz, 1H), 3.73-3.57 (m, 2H), 3.47 (s, 3H), 3.42-3.37 (m, 1H), 3.16-3.03 (m, 4H), 2.45-2.28 (m, 2H), 2.28-2.11 (m, 5H), 1.36 (d, J=6.9 Hz, 3H). Exchangeable proton not observed.

### Example 47: Synthesis of N,N-Dimethyl-2-((2-(3-(3-((4-methyl-4H-1,2,4-triazol-3-yl)methyl)oxetan-3-yl)phenyl)-7-(trifluoromethyl)-1H-benzo[d]imidazol-6-yl)oxy)ethan-1-amine (AZ-1)

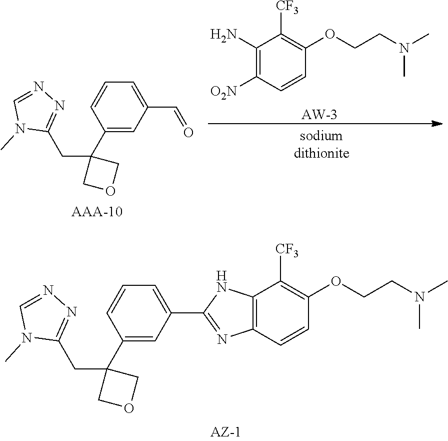

To a stirred solution of intermediate (AAA-10) (28 mg, 1 Eq, 0.11 mmol) in MeOH (10 mL) was added a solution of 1 M sodium dithionite (0.19 g, 1.1 mL, 1 molar, 10 Eq, 1.1 mmol) and intermediate (AW-3) (32 mg, 1 Eq, 0.11 mmol) and stirred for 1 h at 65° C. and at rt overnight. The reaction was concentrated in vacuo and purified by chromatography on RP Flash C18 (12 g cartridge, 15-50% MeCN/10 mM ammonium bicarbonate) to afford the title compound (AZ-1) (4 mg, 8 μmol, 7%, 99% Purity) as a white solid. m/z 501.0 (M+H)⁺ (ES+). ¹H NMR (400 MHz, DMSO-d6) δ 8.18 (s, 1H), 8.08 (d, J=7.8 Hz, 1H), 7.94-7.68 (m, 2H), 7.44 (t, J=7.7 Hz, 1H), 7.19 (d, J=8.9 Hz, 1H), 7.05 (d, J=7.6 Hz, 1H), 4.99 (d, J=6.1 Hz, 2H), 4.95 (d, J=6.1 Hz, 2H), 4.20 (t, J=5.9 Hz, 2H), 3.55 (s, 2H), 2.94 (s, 3H), 2.67 (t, J=5.8 Hz, 2H), 2.24 (s, 6H). Exchangeable proton not observed.

### Example 48: Synthesis of 6-Bromo-2-(3-(3-((4-methyl-4H-1,2,4-triazol-3-yl)methyl)oxetan-3-yl)phenyl)-7-(trifluoromethyl)-1H-benzo[d]imidazole (BA-1)

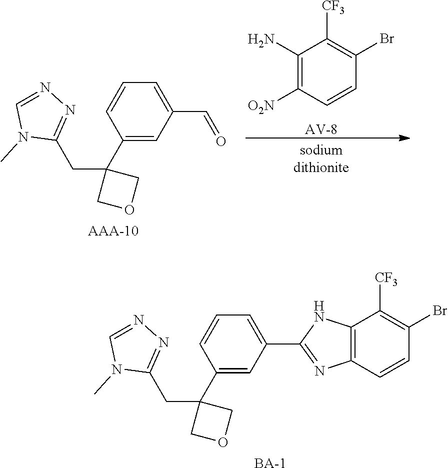

To a stirred solution of intermediate (AAA-10) (28 mg, 1 Eq, 0.11 mmol) in MeOH (2 mL) was added a solution of 1 M sodium dithionite (0.19 g, 1.1 mL, 1 molar, 10 Eq, 1.1 mmol) and 3-bromo-6-nitro-2-(trifluoromethyl)aniline (AV-8) (34 mg, 1.1 Eq, 0.12 mmol) at rt. The resulting mixture was stirred for 1 h at 65° C. followed by rt overnight. The mixture was diluted with water (20 mL) and extracted with DCM (3×15 mL). The organic extracts were dried (MgSO₄), filtered and concentrated under reduced pressure. The crude product was purified by chromatography on silica gel (12 g cartridge, 0-30% MeOH/DCM) to afford the title compound (BA-1) (20 mg, 40 μmol, 37%, 99% Purity) as a clear white solid. m/z 492.2 and 494.2 (M+H)⁺ (ES+). ¹H NMR (400 MHz, Methanol-d₄) δ 8.19 (s, 1H), 8.09 (ddd, J=7.9, 1.8, 1.1 Hz, 1H), 7.81 (s, 1H), 7.76 (d, J=8.7 Hz, 1H), 7.67 (d, J=8.6 Hz, 1H), 7.52 (t, J=7.8 Hz, 1H), 7.08 (ddd, J=7.8, 1.9, 1.1 Hz, 1H), 5.20-5.09 (m, 4H), 3.71 (s, 2H), 2.96 (s, 3H).

### Example 49: Synthesis of 6-Bromo-2-(2′-(4-methyl-4H-1,2,4-triazol-3-yl)-[1,1′-biphenyl]-3-yl)-7-(trifluoromethyl)-1H-benzo[d]imidazole (BB-1)

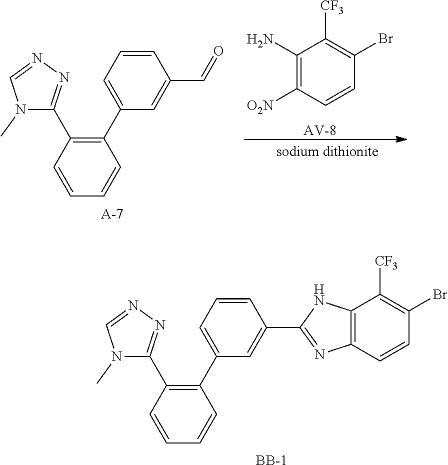

To a stirred solution of intermediate (A-7) (30 mg, 1 Eq, 0.11 mmol) in MeOH (2 mL) was added a solution of 1 M sodium dithionite (0.20 g, 1.1 mL, 1 molar, 10 Eq, 1.1 mmol) and 3-bromo-6-nitro-2-(trifluoromethyl)aniline (AV-8) (32 mg, 1 Eq, 0.11 mmol) at rt. The resulting mixture was stirred for 1 h at 65° C. and at rt overnight. The reaction mixture was diluted with water (5 mL) and extracted with DCM (3×10 mL). The organic extracts were combined, dried (phase separator), and concentrated in vacuo. The crude was purified by preparative HPLC (Waters, Acidic (0.1% formic acid), Acidic, Waters X-Select CSH C18 ODB, 5 μm, 30×100 mm column, 0-100 MeCN in water) to afford the title compound (BB-1) (7.6 mg, 15 μmol, 13%, 98% Purity) as a tan solid. m/z 498.4/500.4 (M+H)⁺ (ES+). ¹H NMR (400 MHz, DMSO-d6) δ 8.38 (s, 1H), 8.15 (d, J=7.3 Hz, 2H), 7.80-7.70 (m, 3H), 7.66-7.59 (m, 3H), 7.50-7.44 (m, 1H), 7.16-7.11 (m, 1H), 3.10 (s, 3H). Exchangeable proton not observed.

### Example 50: Synthesis of 6-Fluoro-2-(2′-(4-methyl-4H-1,2,4-triazol-3-yl)-[1,1′-biphenyl]-3-yl)-7-(trifluoromethyl)-1H-benzo[d]imidazole (BC-1)

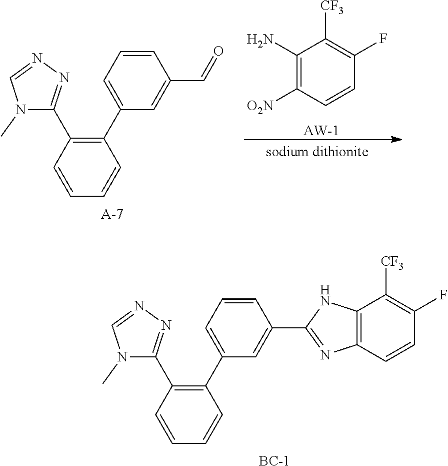

To a stirred solution of intermediate (A-7) (30 mg, 1 Eq, 0.11 mmol) in MeOH (2 mL) was added a solution of 1 M sodium dithionite (0.20 g, 1.1 mL, 1 molar, 10 Eq, 1.1 mmol) and 3-fluoro-6-nitro-2-(trifluoromethyl)aniline (AW-1) (26 mg, 1 Eq, 0.11 mmol) at rt. The resulting mixture was stirred for 1 h at 65° C. and at rt for 3 h. The mixture was filtered through a celite pad and the solvent was removed in vacuo. The crude was purified by preparative HPLC (Waters, Acidic (0.1% formic acid), Acidic, Water X-Select CSH C18 ODB, 5 μm, 30×100 mm column, 0-100% MeCN in water to afford the title compound (BC-1) (12 mg, 27 μmol, 24%, 99% Purity) as white solid. m/z 438.3 (M+H)⁺ (ES+). ¹H NMR (400 MHz, Methanol-d4) δ 8.53 (s, 1H), 8.16 (t, J=1.8 Hz, 1H), 8.12 (dt, J=7.8, 1.4 Hz, 1H), 7.90-7.86 (m, 1H), 7.85-7.71 (m, 2H), 7.69 (d, J=2.5 Hz, 2H), 7.53 (t, J=7.8 Hz, 1H), 7.33-7.19 (m, 2H), 3.26 (s, 3H). Exchangeable proton not observed.

### Example 51: Synthesis of 6-Methoxy-2-(2′-(4-methyl-4H-1,2,4-triazol-3-yl)-[1,1′-biphenyl]-3-yl)-7-(trifluoromethyl)-1H-benzo[d]imidazole (BD-2)

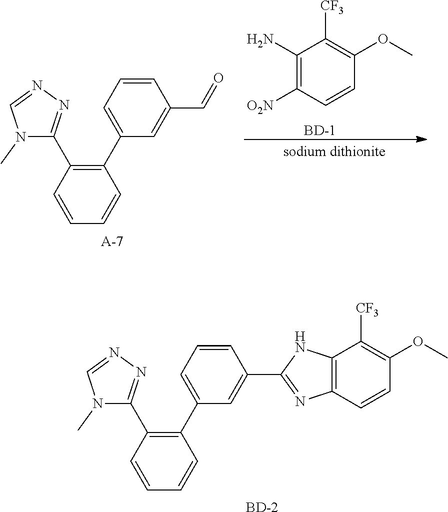

To a stirred solution of intermediate (A-7) (30 mg, 1 Eq, 0.11 mmol) in MeOH (2 mL) was added a solution of 1 M sodium dithionite (0.20 g, 1.1 mL, 1 molar, 10 Eq, 1.1 mmol) and 3-methoxy-6-nitro-2-(trifluoromethyl)aniline (BD-1) (27 mg, 1 Eq, 0.11 mmol) at rt. The resulting mixture was stirred for 1 h at 65° C. and at rt overnight. The reaction mixture was diluted with water (5 mL) and extracted with DCM (3×10 mL). The organic extracts were combined, dried (phase separator), and concentrated in vacuo. The crude product was purified by preparative HPLC (Waters, Acidic (0.1% formic acid), Acidic, Waters X-Select CSH C18 ODB, 5 μm, 30×100 mm, 0-100% MeCN in water to afford the title compound (BD-2) (17.0 mg, 36 μmol, 32%, 95% Purity) as a tan solid. m/z 450.3 (M+H)⁺ (ES+). ¹H NMR (400 MHz, Methanol-d4) δ 8.38 (s, 1H), 8.17-8.13 (m, 1H), 8.10-8.07 (m, 1H), 7.86 (d, J=9.0 Hz, 1H), 7.82-7.79 (m, 2H), 7.69-7.65 (m, 2H), 7.48 (t, J=7.8 Hz, 1H), 7.26-7.20 (m, 2H), 4.00 (s, 3H), 3.19 (s, 3H). One exchangeable proton not observed.

### Example 52: Synthesis of 2-(2′-(4-Methyl-4H-1,2,4-triazol-3-yl)-[1,1′-biphenyl]-3-yl)-7-(trifluoromethyl)-1H-benzo[d]imidazol-6-amine (BE-2)

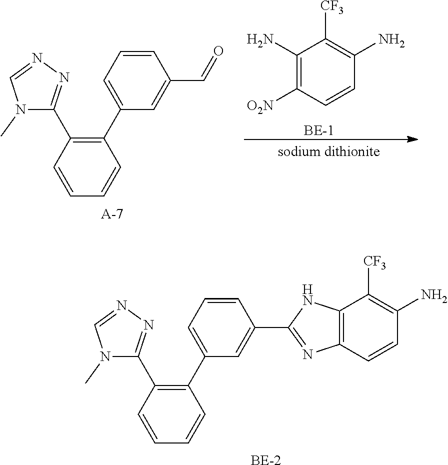

To a stirred solution of 4-nitro-2-(trifluoromethyl)benzene-1,3-diamine (BE-1) (25 mg, 1 Eq, 0.11 mmol) in MeOH (2 mL) was added a solution of intermediate (A-7) (30 mg, 1 Eq, 0.11 mmol) and 1 M sodium dithionite (0.20 g, 1.1 mL, 1 molar, 10 Eq, 1.1 mmol) at rt. The resulting mixture was stirred for 1 h at 65° C. and at rt for 3 h. The mixture was filtered through a celite pad and the solvent was removed in vacuo. The crude was purified by preparative HPLC (Waters, Acidic(0.1% formic acid), Acidic, Waters X-Select CSH C18 ODB, 5 μm, 30×100 mm column, 0-100% MeCN in water to afford the title compound (BE-2) (14 mg, 31 μmol, 27%, 95% Purity) m/z 435.4 (M+H)⁺ (ES+). ¹H NMR (400 MHz, Methanol-d4) δ 8.44 (s, 1H), 8.10 (s, 1H), 8.03 (d, J=7.9 Hz, 1H), 7.83-7.78 (m, 2H), 7.68 (dd, J=5.3, 3.1 Hz, 3H), 7.51 (t, J=7.8 Hz, 1H), 7.26 (d, J=7.7 Hz, 1H), 6.96 (d, J=8.9 Hz, 1H), 3.25 (s, 3H). 3 exchangeable protons not observed.

### Example 53: Synthesis of 1-Methyl-N-(2-(2′-(4-methyl-4H-1,2,4-triazol-3-yl)-[1,1′-biphenyl]-3-yl)-7-(trifluoromethyl)-1H-benzo[d]imidazol-6-yl)piperidine-4-carboxamide, Formic Acid (BF-3)

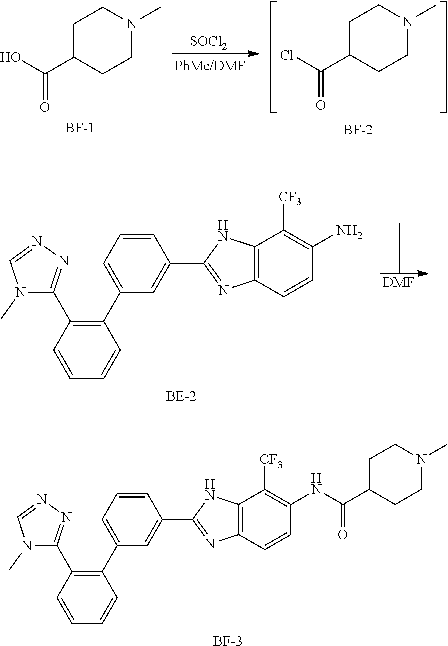

To a solution of 1-methylpiperidine-4-carboxylic acid (BF-1) (4.9 mg, 1.5 Eq, 35 μmol) in toluene (1 mL) were added DMF (0.17 mg, 0.18 μL, 0.1 Eq, 2.3 μmol) and SOCl₂ (8.2 mg, 5.0 μL, 3 Eq, 69 μmol) at rt. The solution was stirred for 5 h at 50° C. The solvent was removed in vacuo, affording acid chloride (BF-2) as a white powder. To a solution of compound (BE-2) (10 mg, 1 Eq, 23 μmol) in DMF (1 mL) was added the acid chloride (BF-2) in DMF (1 mL). The resulting mixture was stirred overnight at 50° C. The reaction mixture was concentrated under reduced pressure. The crude product was purified by chromatography on RP Flash C18(12 g cartridge, 15-75% (0.1% formic acid in MeCN)/(0.1% formic acid in water)) to afford the title compound (BF-3) (1.87 mg, 3.1 μmol, 13%, 99% Purity) as a pale yellow solid. m/z 560.1 (M+H)⁺ (ES+). ¹H NMR (400 MHz, Methanol-d4) δ 8.38 (s, 1H), 8.16 (s, 1H), 8.12 (s, 1H), 7.87 (s, 1H), 7.85-7.76 (m, 2H), 7.70-7.63 (m, 2H), 7.52 (t, J=7.8 Hz, 1H), 7.30 (t, J=8.3 Hz, 2H), 3.53 (d, J=12.1 Hz, 2H), 3.22 (s, 3H), 3.02 (m, 2H), 2.84 (m, 4H), 2.20 (t, J=8.8 Hz, 2H), 2.10 (t, J=12.3 Hz, 2H). Two exchangeable protons not observed.

### Example 54: Synthesis of 2-(2′-(4-Methyl-4H-1,2,4-triazol-3-yl)-[1,1′-biphenyl]-3-yl)-6-(2-(pyrrolidine-1-yl)ethoxy)-7-(trifluoromethyl)-1H-benzo[d]imidazole (BG-1)

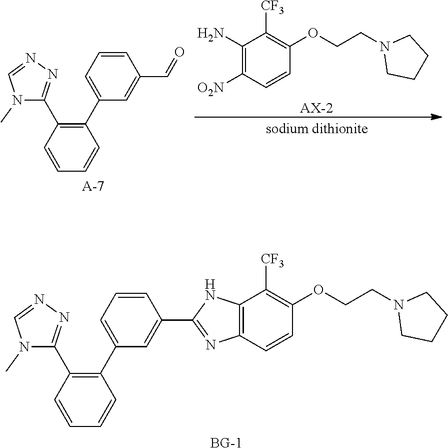

To a stirred solution of intermediate (A-7) (30 mg, 90% Wt, 1 Eq, 0.10 mmol) in MeOH (5 mL) was added a solution of 1M sodium dithionite (0.18 g, 1.0 mL, 1 molar, 10 Eq, 1.0 mmol) and intermediate (AX-2) (33 mg, 1 Eq, 0.10 mmol) at rt. The resulting mixture was stirred for 1 h at 65° C. and at rt overnight. The crude was evaporated and purified by preparative HPLC (Waters, Basic (0.3% ammonia), Basic, Waters XBridge BEH C18 ODB, 5 μm, 30×100 mm column 0-100% MeCN in water to afford the title compound (BG-1) (12 mg, 22 μmol, 22%, 99% Purity). m/z 533.2 (M+H)⁺ (ES+). ¹H NMR (400 MHz, DMSO-d6) δ 8.38 (s, 1H), 8.16 (s, 1H), 8.13 (d, J=7.9 Hz, 1H), 7.79 (d, J=9.1 Hz, 1H), 7.75-7.69 (m, 2H), 7.64-7.57 (m, 2H), 7.42 (t, J=7.8 Hz, 1H), 7.18 (d, J=9.0 Hz, 1H), 7.07 (d, J=7.7 Hz, 1H), 4.21 (t, J=6.0 Hz, 2H), 3.07 (s, 3H), 2.83 (t, J=6.0 Hz, 2H), 2.52-2.46 (m, 2H), 1.74-1.61 (m, 4H). 2H under DMSO, 1H exchangeable not observed.

### Example 55: Synthesis of (R)-2-(2′-(4-Methyl-4H-1,2,4-triazol-3-yl)-[1,1′-biphenyl]-3-yl)-6-((1-methylpyrrolidin-3-yl)oxy)-7-(trifluoromethyl)-1H-benzo[d]imidazole (BH-3)

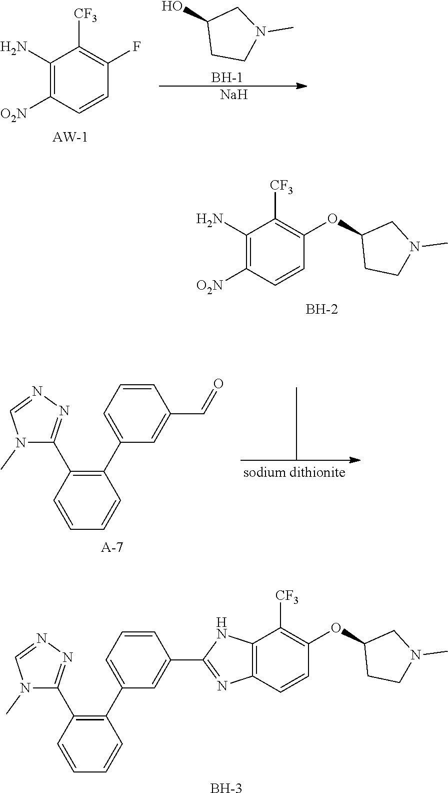

Step 1: (R)-3-((1-Methylpyrrolidin-3-yl)oxy)-6-nitro-2-(trifluoromethyl)aniline (BH-2). To a solution of (R)-1-methylpyrrolidin-3-ol (BH-1) (67.7 mg, 69 μL, 1.5 Eq, 669 μmol) in THF (2 mL) was added NaH (28.6 mg, 60% Wt, 1.6 Eq, 714 μmol) at rt under nitrogen. A solution of 3-fluoro-6-nitro-2-(trifluoromethyl)aniline (AW-1) (100 mg, 1 Eq, 446 μmol) in THF (1 mL) was added and the mixture was stirred at rt overnight. The reaction was partitioned between EtOAc (10 mL) and water (10 mL). The organic layer was washed with water (10 mL), dried (MgSO₄) and concentrated in vacuo to afford the sub-title compound (BH-2) (121 mg, 0.36 mmol, 80%, 90% Purity) as a yellow oil. m/z 306.1 (M+H)⁺ (ES+). ¹H NMR (400 MHz, DMSO-d6) δ 8.30 (d, J=9.7 Hz, 1H), 7.53 (s, 2H), 6.54 (d, J=9.7 Hz, 1H), 5.09 (ddd, J=9.2, 5.9, 2.7 Hz, 1H), 2.84 (dd, J=10.7, 6.1 Hz, 1H), 2.67-2.60 (m, 1H), 2.57 (dd, J=10.6, 2.7 Hz, 1H), 2.42-2.34 (m, 1H), 2.34-2.27 (m, 1H), 2.25 (s, 3H), 1.82-1.73 (m, 1H).

Step 2: (R)-2-(2′-(4-Methyl-4H-1,2,4-triazol-3-yl)-[1,1′-biphenyl]-3-yl)-6-((1-methylpyrrolidin-3-yl)oxy)-7-(trifluoromethyl)-1H-benzo[d]imidazole (BH-3). To a stirred solution of intermediate (A-7) (30 mg, 90% Wt, 1 Eq, 0.10 mmol) in MeOH (10 mL) was added a solution of 1M sodium dithionite (0.18 g, 1.0 mL, 1 molar, 10 Eq, 1.0 mmol) and the product from step 1 above (BH-2) (31 mg, 1 Eq, 0.10 mmol) at rt. The resulting mixture was stirred for 1 h at 65° C. and at rt overnight. The crude was evaporated, dissolved in DMSO (1 mL), filtered through cotton and purified by chromatography on RP Flash C18 (12 g cartridge, 15-50% MeCN/10 mM ammonium bicarbonate) to afford the title compound (BH-3) (11 mg, 21 μmol, 20%, 99% Purity) as a white solid. m/z 519.4 (M+H)⁺ (ES+). ¹H NMR (400 MHz, DMSO-d6) δ 8.37 (s, 1H), 8.19-8.07 (m, 2H), 7.79-7.69 (m, 3H), 7.64-7.58 (m, 2H), 7.42 (t, J=7.7 Hz, 1H), 7.07 (d, J=9.0 Hz, 2H), 5.07-4.97 (m, 1H), 3.07 (s, 3H), 2.92 (dd, J=10.3, 6.3 Hz, 1H), 2.63-2.55 (m, 2H), 2.47-2.41 (m, 1H), 2.27 (s, 4H), 1.89-1.80 (m, 1H). One exchangeable proton not observed.

### Example 56: Synthesis of 2-(4′-Fluoro-2′-(4-methyl-4H-1,2,4-triazol-3-yl)-[1,1′-biphenyl]-3-yl)-7-(trifluoromethyl)-1H-benzo[d]imidazole (BI-1)

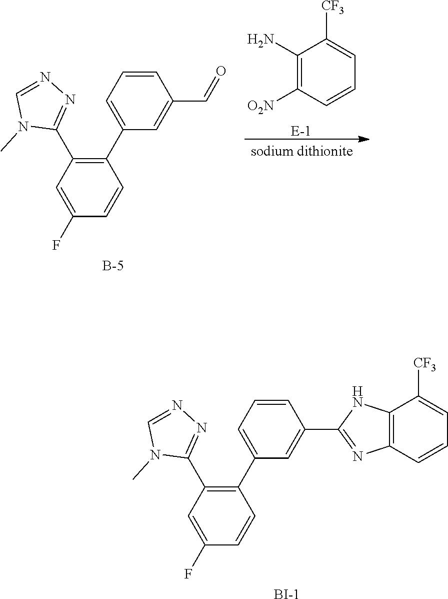

To a stirred solution of intermediate (B-5) (25 mg, 90% Wt, 1 Eq, 80 μmol) in MeOH (2 mL) was added a solution of 1M sodium dithionite (0.14 g, 0.80 mL, 1 molar, 10 Eq, 0.80 mmol) and 2-nitro-6-(trifluoromethyl)aniline (E-1) (21 mg, 80% Wt, 1 Eq, 80 μmol) at rt. The resulting mixture was stirred for 1 h at 65° C. and at rt overnight. The reaction mixture was diluted with water (5 mL) and extracted with DCM (3×10 mL). The organic extracts were combined, dried (phase separator), and concentrated under reduced pressure. The crude product was purified by chromatography on RP Flash C18 (4 g cartridge, 0-100% MeCN/10 mM ammonium bicarbonate) to afford the title compound (BI-1) (1.83 mg, 4.1 μmol, 5.1%, 98% Purity) as a white solid. m/z 438.1 (M+H)⁺ (ES+). ¹H NMR (400 MHz, Methanol-d4) δ 8.39 (s, 1H), 8.20-8.17 (m, 1H), 8.14 (d, J=7.9 Hz, 1H), 7.91-7.81 (m, 2H), 7.62-7.54 (m, 2H), 7.54-7.46 (m, 2H), 7.43 (t, J=7.9 Hz, 1H), 7.26-7.20 (m, 1H), 3.23 (s, 3H). One exchangeable proton not observed.

### Example 57: Synthesis of (2-(4′-Fluoro-2′-(4-methyl-4H-1,2,4-triazol-3-yl)-[1,1′-biphenyl]-3-yl)-7-(trifluoromethyl)-1H-benzo[d]imidazol-5-yl)methanol (BJ-1)

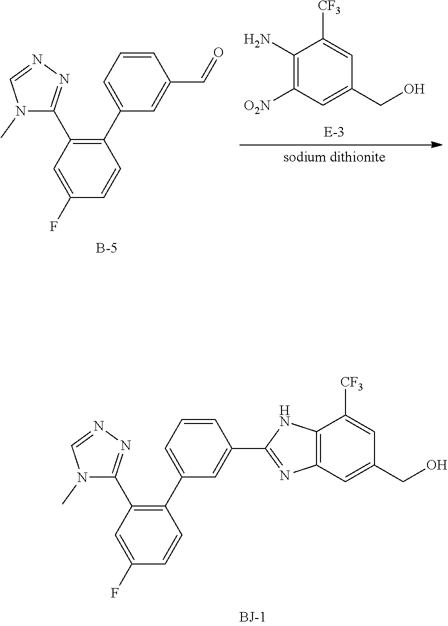

To a stirred solution of intermediate (B-5) (50.0 mg, 90% Wt, 1 Eq, 160 μmol) in MeOH (2 mL) was added a solution of 1M sodium dithionite (279 mg, 1.60 mL, 1 molar, 10 Eq, 1.60 mmol) and intermediate (E-3) (44.4 mg, 85% Wt, 1 Eq, 160 μmol) at rt. The resulting mixture was stirred for 1 h at 65° C. and at rt overnight. The reaction mixture was diluted with EtOAc (25 mL), washed with sat. aq. sol. of NH₄Cl (5 mL), sat. aq. sol. of NaHCO₃ (5 mL) and brine (5 mL). The organic extract was dried (MgSO₄), filtered and concentrated in vacuo. The crude product was purified by chromatography on RP Flash C18 (4 g cartridge, 0-100% MeCN/10 mM ammonium bicarbonate) to afford the title compound (BJ-1) (37 mg, 75 μmol, 47%, 95% Purity) as a white solid. m/z 468.1 (M+H)⁺ (ES+). ¹H NMR (400 MHz, Methanol-d4) δ 8.39 (s, 1H), 8.22-8.08 (m, 2H), 7.84 (dd, J=8.7, 5.5 Hz, 2H), 7.62 (s, 1H), 7.57 (td, J=8.5, 2.8 Hz, 1H), 7.52-7.45 (m, 2H), 7.25-7.20 (m, 1H), 4.81 (s, 2H), 3.23 (s, 3H). Two exchangeable protons not observed.

### Example 58: Synthesis of (1R,2R)-2-(((2-(4′-Fluoro-2′-(4-methyl-4H-1,2,4-triazol-3-yl)-[1,1′-biphenyl]-3-yl)-7-(trifluoromethyl)-1H-benzo[d]imidazol-5-yl)methyl)amino)cyclopentan-1-ol (BK-2)

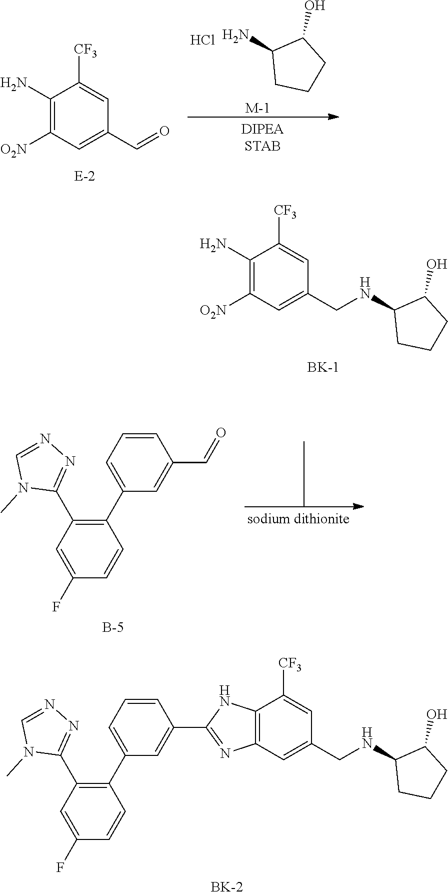

Step 1: (1R,2R)-2-((4-Amino-3-nitro-5-(trifluoromethyl)benzyl)amino)cyclopentan-1-ol (BK-1). NaBH(OAc)₃ (291.0 mg, 3 Eq, 1.373 mmol) was added to a stirred solution of intermediate (E-2) (112.8 mg, 95% Wt, 1 Eq, 457.7 μmol), (1R,2R)-2-aminocyclopentan-1-ol, HCl (M-1) (94.47 mg, 1.5 Eq, 686.5 μmol) and DIPEA (163 mg, 0.217 mL, 2.76 Eq, 1.26 mmol) in THF (5 mL) and stirred at rt overnight. The reaction mixture was concentrated in vacuo. The crude product was purified by chromatography on silica gel (12 g cartridge, 0-10% MeOH/DCM) to afford the sub-title compound (BK-1) (130.5 mg, 0.33 mmol, 71%, 80% Purity) as a bright yellow solid. m/z 320.2 (M+H)⁺ (ES+). ¹H NMR (400 MHz, DMSO-d6) δ 8.56 (s, 1H), 8.10 (s, 1H), 7.51 (s, 2H), 4.17 (s, 3H), 3.23-3.07 (m, 1H), 2.10-1.99 (m, 1H), 1.96-1.84 (m, 1H), 1.70-1.45 (m, 4H), 1.30-1.22 (m, 2H).

Step 2: (1R,2R)-2-(((2-(4′-Fluoro-2′-(4-methyl-4H-1,2,4-triazol-3-yl)-[1,1′-biphenyl]-3-yl)-7-(trifluoromethyl)-1H-benzo[d]imidazol-5-yl)methyl)amino)cyclopentan-1-ol (BK-2). To a stirred solution of intermediate (B-5) (25 mg, 90% Wt, 1 Eq, 80 μmol) in MeOH (2 mL) was added a solution of 1M sodium dithionite (0.14 g, 0.80 mL, 1 molar, 10 Eq, 0.80 mmol) and the product from step 1 above (BK-1) (48 mg, 80% Wt, 1.5 Eq, 0.12 mmol) at rt. The resulting mixture was stirred for 1 h at 65° C. and at rt overnight. The reaction mixture was diluted with water (5 mL) and extracted with DCM (3×10 mL). The organic extracts were combined, dried (phase separator), and concentrated in vacuo. The crude was dissolved in MeOH (10 mL) and loaded onto SCX (ca. 0.5 g) and washed with MeOH (20 mL), the product was then eluted with 0.7 M NH₃/MeOH (20 mL) and concentrated in vacuo. The crude product was purified by preparative HPLC (Waters, Basic (0.3% ammonia), Basic, Waters X-Bridge BEH C18 ODB, 5 μm, 30×100 mm column, 0-100% MeCN in Water) to afford the title compound (BK-2) (6.02 mg, 10 μmol, 13%, 95% Purity) as a white solid. m/z 551.1 (M+H)⁺ (ES+). ¹H NMR (400 MHz, Methanol-d4) δ 8.38 (s, 1H), 8.20-8.15 (m, 1H), 8.13-8.09 (m, 1H), 7.88-7.80 (m, 2H), 7.65 (s, 1H), 7.56 (td, J=8.5, 2.7 Hz, 1H), 7.51-7.44 (m, 2H), 7.23-7.19 (m, 1H), 4.02-3.96 (m, 3H), 3.22 (s, 3H), 2.98-2.91 (m, 1H), 2.12-1.95 (m, 2H), 1.79-1.69 (m, 2H), 1.63-1.54 (m, 1H), 1.50-1.40 (m, 1H). Three exchangeable protons not observed.

### Example 59: Synthesis of (R)-4-((2-(4′-Fluoro-2′-(4-methyl-4H-1,2,4-triazol-3-yl)-[1,1′-biphenyl]-3-yl)-7-(trifluoromethyl)-1H-benzo[d]imidazol-5-yl)methyl)-2-methylmorpholine (BL-2)

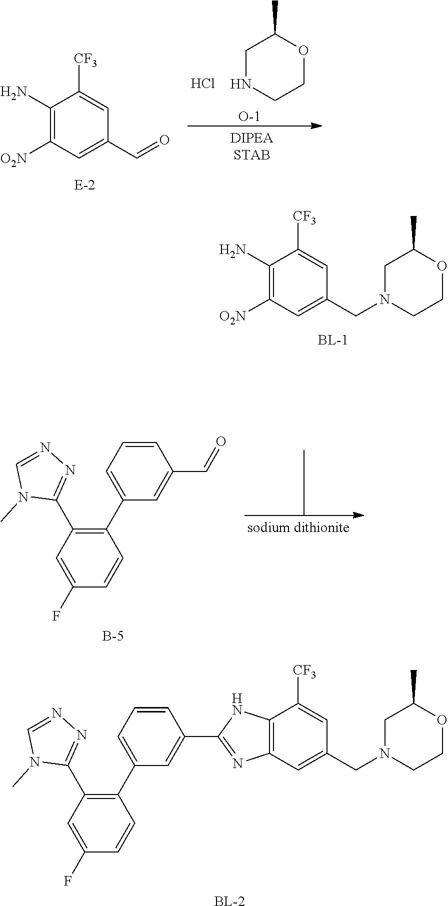

Step 1: (R)-4-((2-Methylmorpholino)methyl)-2-nitro-6-(trifluoromethyl)aniline (BL-1). NaBH(OAc)₃ (258 mg, 3 Eq, 1.22 mmol) was added to a stirred solution of intermediate (E-2) (100 mg, 95% Wt, 1 Eq, 406 μmol), (R)-2-methylmorpholine (0-1) (41.0 mg, 1 Eq, 406 μmol) and DIPEA (163 mg, 0.217 mL, 3.11 Eq, 1.26 mmol) in THF (5 mL) and stirred at rt overnight. The reaction mixture was concentrated in vacuo. The crude product was purified by chromatography on silica gel (12 g cartridge, 0-10% MeOH/DCM) to afford the sub-title compound (BL-1) (51.2 mg, 0.15 mmol, 38%, 95% Purity) as a bright yellow solid. m/z 320.0 (M+H)⁺ (ES+). ¹H NMR (400 MHz, Methanol-d4) δ 8.34 (d, J=2.2 Hz, 1H), 7.84 (d, J=2.1 Hz, 1H), 3.89-3.82 (m, 1H), 3.73-3.62 (m, 2H), 3.50 (s, 2H), 2.80-2.74 (m, 1H), 2.72-2.67 (m, 1H), 2.19 (td, J=11.5, 3.4 Hz, 1H), 1.92-1.82 (m, 1H), 1.14 (d, J=6.3 Hz, 3H). Two exchangeable protons not observed.

Step 2: (R)-4-((2-(4′-Fluoro-2′-(4-methyl-4H-1,2,4-triazol-3-yl)-[1,1′-biphenyl]-3-yl)-7-(trifluoromethyl)-1H-benzo[d]imidazol-5-yl)methyl)-2-methylmorpholine (BL-2). To a stirred solution of intermediate (B-5) (36.6 mg, 90% Wt, 1 Eq, 117 μmol in MeOH (2 mL) was added a solution of 1M sodium dithionite (204 mg, 1.17 mL, 1 molar, 10 Eq, 1.17 mmol) and the product from step 1 above (BL-1) (41.5 mg, 90% Wt, 1 Eq, 117 μmol) at rt. The resulting mixture was stirred for 1 h at 65° C. and at rt overnight. The reaction mixture was diluted with water (5 mL) and extracted with DCM (3×10 mL). The organic extracts were combined, dried (phase separator), and concentrated in vacuo. The crude product was purified by chromatography on RP Flash C18(4 g cartridge, 0-100% MeCN/10 mM ammonium bicarbonate) to afford the title compound (BL-2) (6 mg, 0.01 mmol, 9%, 95% Purity) as a pale yellow solid. m/z 551.4 (M+H)⁺ (ES+). ¹H NMR (400 MHz, Methanol-d4) δ 8.39 (s, 1H), 8.19-8.09 (m, 2H), 7.87-7.82 (m, 1H), 7.62 (s, 1H), 7.60-7.54 (m, 2H), 7.54-7.45 (m, 2H), 7.24 (d, J=7.8 Hz, 1H), 3.90-3.84 (m, 1H), 3.72 (s, 2H), 3.72-3.64 (m, 1H), 3.23 (s, 3H), 2.82 (d, J=11.2 Hz, 1H), 2.75 (d, J=11.4 Hz, 1H), 2.28-2.19 (m, 1H), 1.96-1.88 (m, 1H), 1.41-1.30 (m, 1H), 1.14 (d, J=6.3 Hz, 3H). One exchangeable proton not observed.

### Example 60: Synthesis of (1S,2R)-2-(((2-(4′-Fluoro-2′-(4-methyl-4H-1,2,4-triazol-3-yl)-[1,1′-biphenyl]-3-yl)-7-(trifluoromethyl)-1H-benzo[d]imidazol-5-yl)methyl)amino)cyclopentan-1-ol (BM-2)

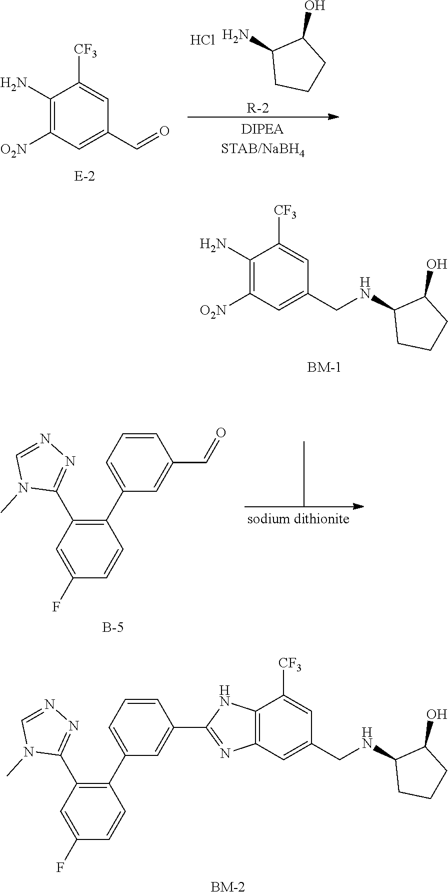

Step 1: (1S,2R)-2-((4-Amino-3-nitro-5-(trifluoromethyl)benzyl)amino)cyclopentan-1-ol (BM-1). NaBH(OAc)₃ (269 mg, 3 Eq, 1.27 mmol) was added to a stirred solution of intermediate (E-2) (100 mg, 99% Wt, 1 Eq, 423 μmol), (1S,2R)-2-aminocyclopentan-1-ol, HCl (R-2) (64.0 mg, 1.1 Eq, 465 μmol) and DIPEA (163 mg, 0.217 mL, 2.99 Eq, 1.26 mmol) in THF (5 mL) and stirred at rt overnight. NaBH₄ (16.0 mg, 1 Eq, 423 μmol) was added to the reaction mixture and allowed to stir at rt for 16 h. The reaction mixture was quenched with sat. aq. sol. of NaHCO₃ (10 mL) and extracted with DCM (3×10 mL). The organic extracts were combined, dried (phase separator) and concentrated in vacuo. The crude product was purified by chromatography on RP Flash C18 (12 g cartridge, 0-100% MeCN/10 mM ammonium bicarbonate) to afford the sub-title compound (BM-1) (99.2 mg, 0.28 mmol, 66%, 90% Purity) as a bright yellow solid. m/z 320.2 (M+H)⁺ (ES+). ¹H NMR (400 MHz, DMSO-d6) δ 8.31-8.22 (m, 1H), 7.87 (dd, J=4.7, 2.2 Hz, 1H), 7.25 (s, 2H), 4.36 (s, 1H), 3.93 (s, 1H), 3.67 (q, J=13.7 Hz, 2H), 2.73 (s, 1H), 1.74-1.53 (m, 3H), 1.47-1.32 (m, 2H). Two exchangeable protons not observed.

Step 2: (1S,2R)-2-(((2-(4′-Fluoro-2′-(4-methyl-4H-1,2,4-triazol-3-yl)-[1,1′-biphenyl]-3-yl)-7-(trifluoromethyl)-1H-benzo[d]imidazol-5-yl)methyl)amino)cyclopentan-1-ol (BM-2). To a stirred solution of intermediate (B-5) (15 mg, 90% Wt, 1 Eq, 48 μmol) in MeOH (2 mL) was added a solution of 1M sodium dithionite (84 mg, 0.48 mL, 1 molar, 10 Eq, 0.48 mmol) and the product from step 1 above (BM-1) (17 mg, 90% Wt, 1 Eq, 48 μmol) at rt. The resulting mixture was stirred for 1 h at 65° C. and at rt overnight. The reaction mixture was diluted with water (5 mL) and extracted with DCM (3×10 mL). The organic extracts were combined, dried (phase separator), and concentrated in vacuo. The crude product was purified by preparative HPLC (Waters, Basic (0.1% ammonium bicarbonate), Basic, Waters X-Bridge Prep-C18, 5 μm, 19×50 mm column, 0-100% MeCN in Water) to afford the title compound (BM-2) (4.21 mg, 7.3 μmol, 15%, 95% Purity) as a white solid m/z 551.2 (M+H)⁺ (ES+). ¹H NMR (400 MHz, DMSO-d6) δ 13.26 (s, 1H), 8.40 (s, 1H), 8.11 (d, J=7.9 Hz, 1H), 8.07 (s, 1H), 7.80-7.75 (m, 2H), 7.66-7.58 (m, 1H), 7.57-7.50 (m, 2H), 7.46 (t, J=7.8 Hz, 1H), 7.14-7.06 (m, 1H), 4.47-4.34 (m, 1H), 4.02-3.82 (m, 3H), 3.16 (s, 3H), 2.85-2.77 (m, 1H), 1.76-1.55 (m, 3H), 1.49-1.36 (m, 2H). Two exchangeable protons not observed.

### Example 61: Synthesis of (1S,2S)-2-(((2-(4′-Fluoro-2′-(4-methyl-4H-1,2,4-triazol-3-yl)-[1,1′-biphenyl]-3-yl)-7-(trifluoromethyl)-1H-benzo[d]imidazol-5-yl)methyl)amino)cyclopentan-1-ol (BN-2)

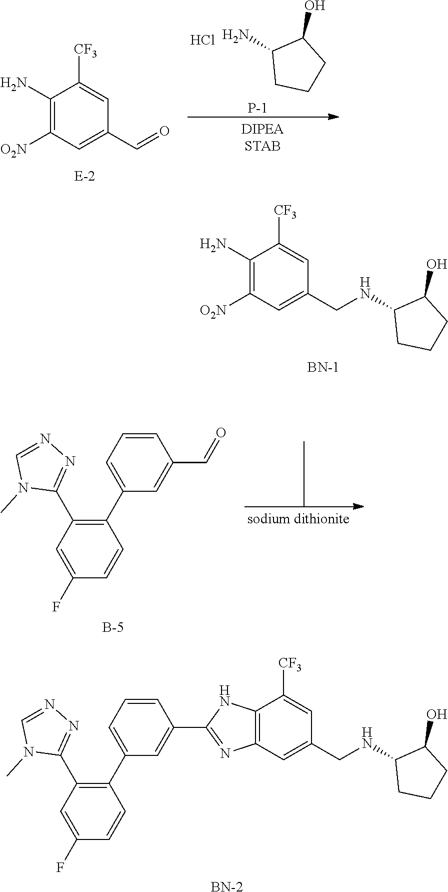

Step 1: (1S,2S)-2-((4-Amino-3-nitro-5-(trifluoromethyl)benzyl)amino)cyclopentan-1-ol (BN-1). NaBH(OAc)₃ (258 mg, 3 Eq, 1.22 mmol) was added to a stirred solution of intermediate (E-2) (100 mg, 95% Wt, 1 Eq, 406 μmol), (1S,2S)-2-aminocyclopentan-1-ol (P-1) (41.0 mg, 1 Eq, 406 μmol) and DIPEA (163 mg, 0.217 mL, 3.11 Eq, 1.26 mmol) in THF (5 mL) and stirred at rt overnight. The reaction mixture was concentrated in vacuo. The crude product was purified by chromatography on silica gel (12 g cartridge, 0-10% MeOH/DCM) to afford the sub-title compound (BN-1) (75 mg, 0.21 mmol, 52%, 90% Purity) as a bright yellow solid. m/z 319.6 (M+H)⁺ (ES+). ¹H NMR (400 MHz, Methanol-d4) δ 8.41 (d, J=2.2 Hz, 1H), 7.90 (d, J=2.2 Hz, 1H), 4.01-3.92 (m, 1H), 3.81 (s, 2H), 2.95-2.84 (m, 1H), 2.14-1.89 (m, 3H), 1.82-1.66 (m, 2H), 1.66-1.52 (m, 1H), 1.52-1.31 (m, 2H). Two exchangeable protons not observed.

Step 2: (1S,2S)-2-(((2-(4′-Fluoro-2′-(4-methyl-4H-1,2,4-triazol-3-yl)-[1,1′-biphenyl]-3-yl)-7-(trifluoromethyl)-1H-benzo[d]imidazol-5-yl)methyl)amino)cyclopentan-1-ol (BN-2). To a stirred solution of intermediate (B-5) (34 mg, 90% Wt, 1 Eq, 0.11 mmol) in MeOH (2 mL) was added a solution of 1M sodium dithionite (0.19 g, 1.1 mL, 1 molar, 10 Eq, 1.1 mmol) and the product from step 1 above (BN-1) (39 mg, 90% Wt, 1 Eq, 0.11 mmol) at rt. The resulting mixture was stirred for 1 h at 65° C. and at rt 2 days. Additional (BN-1) (39 mg, 90% Wt, 1 Eq, 0.11 mmol) and 1M sodium dithionite (0.19 g, 1.1 mL, 1 molar, 10 Eq, 1.1 mmol) in water (1 mL) was added and stirred at 65° C. for 1 h followed by overnight at rt. The reaction mixture was diluted with water (5 mL) and extracted with DCM (3×10 mL). The organic extracts were combined, dried (phase separator), and concentrated in vacuo. The crude product was purified by preparative HPLC (Waters, Basic (0.1% ammonium bicarbonate), Basic, Waters X-Bridge Prep-C18, 5 μm, 19×50 mm column, 0-100% MeCN in water) to afford the title compound (BN-2) (4.5 mg, 7.8 μmol, 7.1%, 95% Purity) as a white solid. m/z 551.1 (M+H)⁺ (ES+). ¹H NMR (400 MHz, DMSO-d6) δ 8.40 (s, 1H), 8.15-8.05 (m, 2H), 7.80-7.72 (m, 2H), 7.62 (td, J=8.5, 2.8 Hz, 1H), 7.55-7.50 (m, 2H), 7.48-7.41 (m, 1H), 7.12-7.04 (m, 1H), 4.49 (d, J=4.1 Hz, 1H), 3.91-3.76 (m, 3H), 3.16 (s, 3H), 2.80-2.75 (m, 1H), 1.88-1.78 (m, 2H), 1.62-1.53 (m, 2H), 1.46-1.24 (m, 2H). Two exchangeable protons not observed.

### Example 62: Synthesis of (S)-2-(4′-Fluoro-2′-(4-methyl-4H-1,2,4-triazol-3-yl)-[1,1′-biphenyl]-3-yl)-5-((3-methylpiperidin-1-yl)methyl)-7-(trifluoromethyl)-1H-benzo[d]imidazole (BO-2)

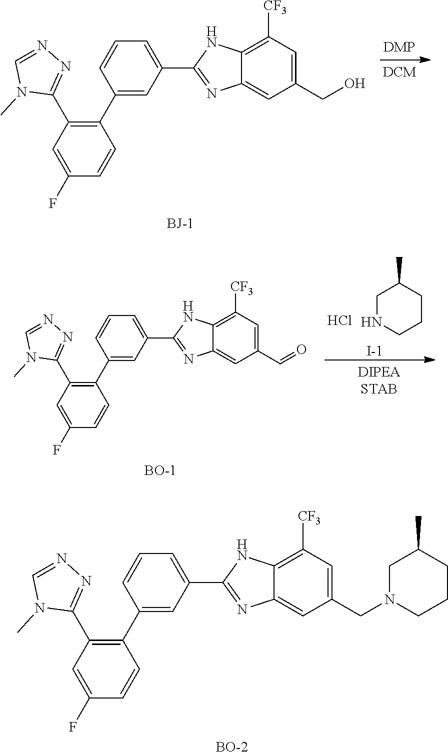

Step 1: 2-(4′-Fluoro-2′-(4-methyl-4H-1,2,4-triazol-3-yl)-[1,1′-biphenyl]-3-yl)-7-(trifluoromethyl)-1H-benzo[d]imidazole-5-carbaldehyde (BO-1). A mixture of compound (BJ-1) (28 mg, 1 Eq, 60 μmol) and Dess-Martin periodinane (38 mg, 1.5 Eq, 90 μmol) was stirred in DCM (2 mL) at 40° C. The reaction mixture was diluted with DCM (10 mL) and washed with sat. aq. NaHCO₃ (10 mL). Organics were separated, dried (MgSO₄), filtered and concentrated in vacuo to afford the sub-title compound (BO-1) (28 mg, 55 μmol, 92%, 92% Purity) as white solid. m/z 466.2 (M+H)⁺ (ES+).

Step 2: (S)-2-(4′-Fluoro-2′-(4-methyl-4H-1,2,4-triazol-3-yl)-[1,1′-biphenyl]-3-yl)-5-((3-methylpiperidin-1-yl)methyl)-7-(trifluoromethyl)-1H-benzo[d]imidazole (BO-2). A solution of the product from step 1 above (BO-1) (25 mg, 1 Eq, 54 μmol), (S)-3-methylpiperidine, HCl (I-1) (11 mg, 1.5 Eq, 81 μmol) and DIPEA (21 mg, 28 μL, 3 Eq, 0.16 mmol) in CHCl₃ (1 mL) was stirred 1 h at rt, then NaBH(OAc)₃ (28 mg, 2.5 Eq, 0.13 mmol) was added. The reaction mixture was stirred at rt overnight. An additional 1 eq. of NaBH(OAc)₃, DIPEA and amine (I-1) were added and the mixture was stirred for 4 h at rt. The reaction mixture was diluted with EtOAc (25 mL) and transferred into a separating funnel. The layer was washed with sat. aq. sol. of NaHCO₃ (2×5 mL) and brine. The combined organic layers were collected, dried (MgSO₄), filtered and concentrated in vacuo. The crude product was purified by chromatography on RP Flash C18 (12 g cartridge, 15-75% MeCN/10 mM ammonium bicarbonate) to afford the title compound (BO-2) (19 mg, 34 μmol, 64%, 99% Purity) as a white solid. m/z 549.2 (M+H)⁺ (ES+). ¹H NMR (400 MHz, Methanol-d4) δ 8.39 (s, 1H), 8.18 (s, 1H), 8.13 (d, J=7.9 Hz, 1H), 7.88-7.80 (m, 2H), 7.61 (s, 1H), 7.57 (td, J=8.5, 2.8 Hz, 1H), 7.53-7.45 (m, 2H), 7.23 (dt, J=7.9, 1.3 Hz, 1H), 3.71 (s, 2H), 3.23 (s, 3H), 2.90 (dd, J=16.1, 9.4 Hz, 2H), 2.01 (td, J=11.5, 3.2 Hz, 1H), 1.82-1.60 (m, 5H), 0.90 (d, J=5.5 Hz, 4H). Exchangeable proton not observed.

### Example 63: Synthesis of 1-((((2-(4′-Fluoro-2′-(4-methyl-4H-1,2,4-triazol-3-yl)-[1,1′-biphenyl]-3-yl)-7-(trifluoromethyl)-1H-benzo[d]imidazol-5-yl)methyl)amino)methyl)cyclobutan-1-ol (BP-1)

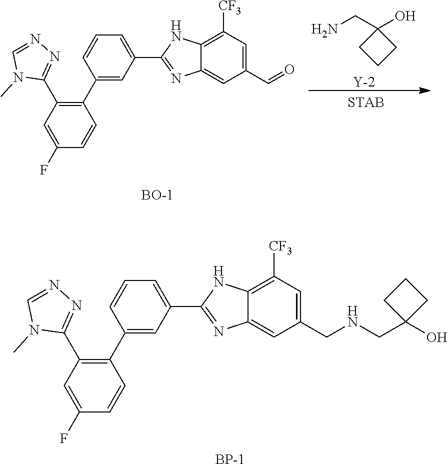

A solution of intermediate (BO-1) (30 mg, 1 Eq, 64 μmol) and 1-(aminomethyl)cyclobutan-1-ol (Y-2) (13 mg, 2.0 Eq, 0.13 mmol) in CHCl₃ (1 mL) was stirred 1h at rt, then NaBH(OAc)₃ (41 mg, 3.0 Eq, 0.19 mmol) was added. The reaction mixture was stirred at rt overnight. An additional 1 eq. of NaBH(OAc)₃ was added and the mixture stirred for 3 h at rt. The reaction mixture was diluted with EtOAc (25 mL) and transferred into a separating funnel. The layer was washed with sat. aq. sol. of NaHCO₃ (2×5 mL) and brine. The combined organic layers were collected, dried (MgSO₄), filtered and concentrated in vacuo. The crude product was purified by preparative HPLC (Waters, Basic (0.3% ammonium bicarbonate), Basic, Waters XBridge BEH C18 ODB, 5 μm, 30×100 mm column, 20-100% MeCN in water) to afford the title compound (BP-1) (5 mg, 9 μmol, 10%, 96% Purity) as a white solid. m/z 551.2 (M+H)⁺ (ES+). ¹H NMR (400 MHz, DMSO-d6) δ 8.45 (s, 1H), 8.24 (t, J=1.8 Hz, 1H), 8.22-8.16 (m, 1H), 7.94 (s, 1H), 7.90 (dd, J=8.6, 5.5 Hz, 1H), 7.72 (s, 1H), 7.63 (td, J=8.5, 2.8 Hz, 1H), 7.59-7.51 (m, 2H), 7.29 (dt, J=7.9, 1.3 Hz, 1H), 4.11 (s, 2H), 3.29 (s, 3H), 2.84 (s, 2H), 2.28-2.06 (m, 4H), 1.91-1.76 (m, 1H), 1.61 (dt, J=11.3, 8.9 Hz, 1H). Exchangeable protons not observed.

### Example 64: Synthesis of 3-((((2-(4′-Fluoro-2′-(4-methyl-4H-1,2,4-triazol-3-yl)-[1,1′-biphenyl]-3-yl)-7-(trifluoromethyl)-1H-benzo[d]imidazol-5-yl)methyl)amino)methyl)oxetan-3-ol (BQ-1)

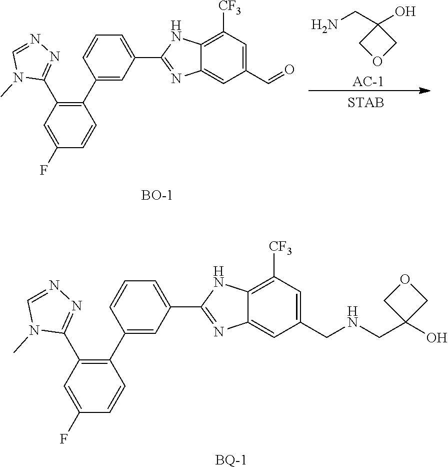

A solution of intermediate (BO-1) (30 mg, 1 Eq, 64 μmol) and 3-(aminomethyl)oxetan-3-ol (AC-1) (13 mg, 2.0 Eq, 0.13 mmol) in CHCl₃ (1 mL) was stirred 1 h at rt, then NaBH(OAc)₃ (41 mg, 3.0 Eq, 0.19 mmol) was added. The reaction mixture was stirred at rt overnight. An additional 1 eq. of NaBH(OAc)₃ was added and the mixture stirred for 3 h at rt. The reaction mixture was diluted with EtOAc (25 mL) and transferred into a separating funnel. The layer was washed with sat. aq. sol. of NaHCO₃ (2×5 mL) and brine. The combined organic layers were collected, dried (MgSO₄), filtered and concentrated in vacuo. The crude product was purified by preparative HPLC (Waters, Basic (0.3% ammonium bicarbonate), Basic, Waters XBridge BEH C18 ODB, 5 μm, 30×100 mm column, 15-100% MeCN in water) to afford the title compound (BQ-1) (6 mg, 0.01 mmol, 20%, 96% Purity). m/z 553.3 (M+H)⁺ (ES+). ¹H NMR (400 MHz, Methanol-d4) δ 8.39 (s, 1H), 8.18 (t, J=1.8 Hz, 1H), 8.13 (dt, J=7.8, 1.4 Hz, 1H), 7.90-7.81 (m, 2H), 7.66 (s, 1H), 7.57 (td, J=8.5, 2.7 Hz, 1H), 7.53-7.43 (m, 2H), 7.26-7.21 (m, 1H), 4.59 (d, J=6.7 Hz, 2H), 4.55 (d, J=6.8 Hz, 2H), 4.04 (s, 2H), 3.23 (s, 3H), 2.98 (s, 2H). Exchangeable protons not observed.

### Example 65: Synthesis of 1-((2-(4′-Fluoro-2′-(4-methyl-4H-1,2,4-triazol-3-yl)-[1,1′-biphenyl]-3-yl)-7-(trifluoromethyl)-1H-benzo[d]imidazol-5-yl)methyl)-3-methylazetidin-3-ol (BR-1)

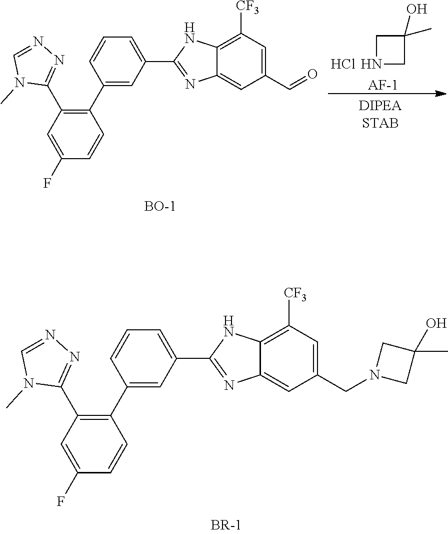

A solution of intermediate (BO-1) (30 mg, 1 Eq, 64 μmol) and 3-methylazetidin-3-ol, HCl (AF-1) (16 mg, 2.0 Eq, 0.13 mmol) in CHCl₃ (1 mL) was stirred 1 h at rt, then NaBH(OAc)₃ (41 mg, 3.0 Eq, 0.19 mmol) was added. The reaction mixture was stirred at rt overnight. An additional 1 eq. of NaBH(OAc)₃ was added and the mixture stirred for 3 h at rt. The reaction mixture was diluted with EtOAc (25 mL) and transferred into a separating funnel. The layer was washed with sat. aq. sol. of NaHCO₃ (2×5 mL) and brine. The combined organic layers were collected, dried (MgSO₄), filtered and concentrated in vacuo. The crude product was purified by preparative HPLC (Waters, Basic (0.3% ammonium bicarbonate), Basic, Waters XBridge BEH C18 ODB, 5 μm, 30×100 mm column, 15-100% MeCN in water) to afford the title compound (BR-1) (7 mg, 0.01 mmol, 20%, 98% Purity) as a white solid. m/z 537.2 (M+H)⁺ (ES+). ¹H NMR (400 MHz, Methanol-d4) δ 8.39 (s, 1H), 8.17 (t, J=1.8 Hz, 1H), 8.13 (dt, J=8.0, 1.4 Hz, 1H), 7.86-7.78 (m, 2H), 7.60-7.53 (m, 2H), 7.53-7.45 (m, 2H), 7.23 (ddd, J=7.8, 1.9, 1.1 Hz, 1H), 3.88 (s, 2H), 3.37 (dd, J=7.1, 1.7 Hz, 2H), 3.23 (s, 3H), 3.21-3.17 (m, 2H), 1.51 (s, 3H). Exchangeable protons not observed.

### Example 66: Synthesis of (3-(((2-(4′-Fluoro-2′-(4-methyl-4H-1,2,4-triazol-3-yl)-[1,1′-biphenyl]-3-yl)-7-(trifluoromethyl)-1H-benzo[d]imidazol-5-yl)methyl)amino)oxetan-3-yl)methanol (BS-1)

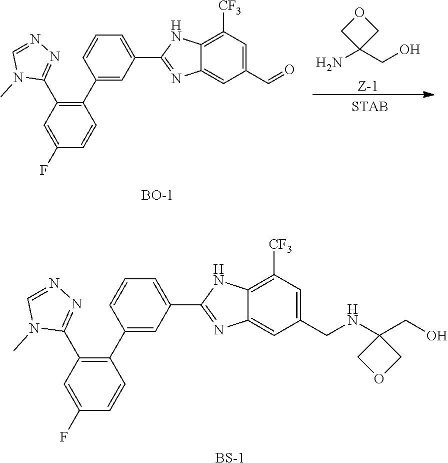

A solution of intermediate (BO-1) (25 mg, 1 Eq, 54 μmol) and (3-aminooxetan-3-yl)methanol (Z-1) (11 mg, 2.0 Eq, 0.11 mmol) in CHCl₃ (1 mL) was stirred 1h at rt, then NaBH(OAc)₃ (34 mg, 3.0 Eq, 0.16 mmol) was added. The reaction mixture was stirred at rt overnight. An additional 1 eq. of NaBH(OAc)₃ was added and the mixture stirred for 3 h at rt. The reaction mixture was diluted with EtOAc (25 mL) and transferred into a separating funnel. The layer was washed with sat. aq. sol. of NaHCO₃ (2×5 mL) and brine. The combined organic layers were collected, dried (MgSO₄), filtered and concentrated in vacuo. The crude product was purified by preparative HPLC (Waters, Basic (0.3% ammonium bicarbonate), Basic, Waters XBridge BEH C18 ODB, 5 μm, 30×100 mm column, 10-100% MeCN in water) to afford the title compound (BS-1) (8 mg, 0.01 mmol, 30%, 96% Purity) as a white solid. m/z 553.2 (M+H)⁺ (ES+). ¹H NMR (400 MHz, Methanol-d4) δ 8.39 (s, 1H), 8.18 (t, J=1.8 Hz, 1H), 8.12 (d, J=8.0 Hz, 1H), 7.91 (s, 1H), 7.84 (dd, J=8.6, 5.5 Hz, 1H), 7.70 (s, 1H), 7.56 (td, J=8.5, 2.7 Hz, 1H), 7.53-7.44 (m, 2H), 7.22 (dt, J=7.9, 1.3 Hz, 1H), 4.69 (d, J=6.5 Hz, 2H), 4.50 (d, J=6.5 Hz, 2H), 4.04 (s, 2H), 3.93 (s, 2H), 3.23 (s, 3H). Exchangeable protons not visible.

### Example 67: Synthesis of 1-(((2-(4′-Fluoro-2′-(4-methyl-4H-1,2,4-triazol-3-yl)-[1,1′-biphenyl]-3-yl)-7-(trifluoromethyl)-1H-benzo[d]imidazol-5-yl)methyl)amino)-2-methylpropan-2-ol (BT-1)

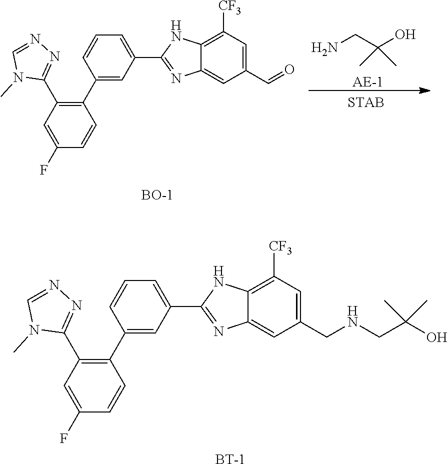

A solution of intermediate (BO-1) (30 mg, 1 Eq, 64 μmol) and 1-amino-2-methylpropan-2-ol (AE-1) (11 mg, 12 μL, 2.0 Eq, 0.13 mmol) in CHCl₃ (1 mL) was stirred 1h at rt, then NaBH(OAc)₃ (41 mg, 3.0 Eq, 0.19 mmol) was added. The reaction mixture was stirred at rt overnight. The An additional 1 eq. of NaBH(OAc)₃ was added and the mixture was stirred for 3 h at rt. The reaction mixture was diluted with EtOAc (25 mL) and transferred into a separating funnel. The layer was washed with sat. aq. sol. of NaHCO₃ (2×5 mL) and brine. The combined organic layers were collected, dried (MgSO₄), filtered and concentrated in vacuo. The crude product was purified by preparative HPLC (Waters, Basic (0.3% ammonium bicarbonate), Basic, Waters XBridge BEH C18 ODB, 5 μm, 30×100 mm column, 20-100% MeCN in water) to afford the title compound (BT-1) (5 mg, 9 μmol, 10%, 97% Purity) as a white solid. m/z 539.2 (M+H)⁺ (ES+). ¹H NMR (400 MHz, DMSO-d6) δ 8.39 (s, 1H), 8.18 (s, 1H), 8.13 (d, J=7.9 Hz, 1H), 7.91-7.78 (m, 2H), 7.65 (s, 1H), 7.57 (td, J=8.5, 2.8 Hz, 1H), 7.53-7.44 (m, 2H), 7.23 (dd, J=7.8, 1.4 Hz, 1H), 4.02 (s, 2H), 3.23 (s, 3H), 2.61 (s, 2H), 1.26 (s, 6H). Exchangeable protons not observed.

### Example 68: Synthesis of 5-((6-Azaspiro[2.5]octan-6-yl)methyl)-2-(4′-fluoro-2′-(4-methyl-4H-1,2,4-triazol-3-yl)-[1,1′-biphenyl]-3-yl)-7-(trifluoromethyl)-1H-benzo[d]imidazole (BU-1)

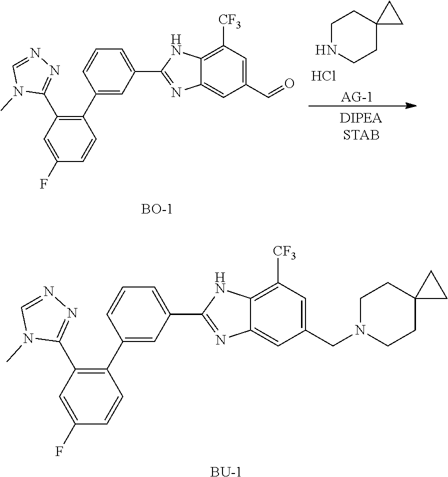

A solution of intermediate (BO-1) (30 mg, 90% Wt, 1 Eq, 58 μmol), 6-azaspiro[2.5]octane, HCl (AG-1) (17 mg, 2.0 Eq, 0.12 mmol) and DIPEA (22 mg, 30 μL, 3.0 Eq, 0.17 mmol) in CHCl₃ (1 mL) was stirred 1 h at 40° C., then NaBH(OAc)₃ (37 mg, 3.0 Eq, 0.17 mmol) was added. The reaction mixture was stirred at 40° C. overnight. An additional 1 eq. of NaBH(OAc)₃ was added and the mixture was stirred for 3 h at rt. The reaction mixture was diluted with EtOAc (25 mL) and transferred into a separating funnel. The layer was washed with sat. aq. sol. of NaHCO₃ (2×5 mL) and brine. The combined organic layers were collected, dried (MgSO₄), filtered and concentrated in vacuo. The crude product was purified by chromatography on silica gel (12 g cartridge, 0-20% MeOH/DCM) to afford the title compound (BU-1) (20 mg, 34 μmol, 58%, 95% Purity) as a clear white solid. m/z 561.2 (M+H)⁺ (ES+). ¹H NMR (400 MHz, Methanol-d4) δ 8.39 (s, 1H), 8.18 (t, J=1.8 Hz, 1H), 8.13 (d, J=7.9 Hz, 1H), 7.87-7.81 (m, 2H), 7.63 (s, 1H), 7.57 (td, J=8.5, 2.8 Hz, 1H), 7.54-7.45 (m, 2H), 7.24 (ddd, J=7.7, 1.9, 1.1 Hz, 1H), 3.77 (s, 2H), 3.23 (s, 3H), 2.59 (s, 4H), 1.49 (s, 4H), 0.34 (s, 4H). Exchangeable proton not visible.

### Example 69: Synthesis of 1-((((2-(4′-Fluoro-2′-(4-methyl-4H-1,2,4-triazol-3-yl)-[1,1′-biphenyl]-3-yl)-7-(trifluoromethyl)-1H-benzo[d]imidazol-5-yl)methyl)amino)methyl)cyclopropan-1-ol (BV-1)

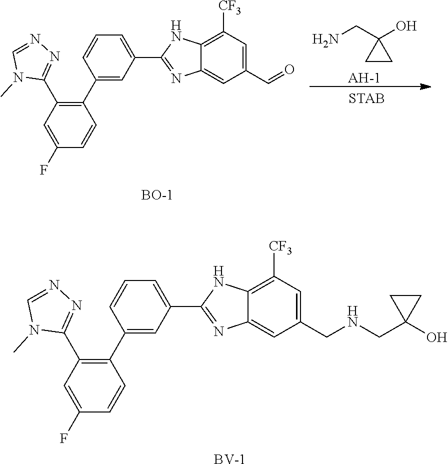

A solution of intermediate (BO-1) (30 mg, 90% Wt, 1 Eq, 58 μmol) and 1-(aminomethyl)cyclopropan-1-ol (AH-1) (10 mg, 2.0 Eq, 0.12 mmol) in CHCl₃ (1 mL) was stirred 1 h at 40° C., then NaBH(OAc)₃ (37 mg, 3.0 Eq, 0.17 mmol) was added. The reaction mixture was stirred at 40° C. overnight. An additional 1 eq. of NaBH(OAc)₃ was added and the mixture was stirred 3 h at 40° C. The reaction mixture was diluted with EtOAc (25 mL) and transferred into a separating funnel. The layer was washed with sat. aq. sol. of NaHCO₃ (2×5 mL) and brine. The combined organic layers were collected, dried (MgSO₄), filtered and concentrated in vacuo. The crude product was purified by chromatography on silica gel (12 g cartridge, 0-20% MeOH/DCM) to afford the title compound (BV-1) (14 mg, 25 μmol, 43%, 96% Purity) as a clear white solid. m/z 537.3 (M+H)⁺ (ES+). ¹H NMR (400 MHz, Methanol-d4) δ 8.39 (s, 1H), 8.18 (s, 1H), 8.13 (dt, J=7.8, 1.4 Hz, 1H), 7.88 (s, 1H), 7.84 (dd, J=8.7, 5.4 Hz, 1H), 7.67 (s, 1H), 7.57 (td, J=8.5, 2.8 Hz, 1H), 7.54-7.45 (m, 2H), 7.23 (ddd, J=7.8, 1.8, 1.0 Hz, 1H), 4.07 (s, 2H), 3.23 (s, 3H), 2.79 (s, 2H), 0.80-0.73 (m, 2H), 0.62-0.52 (m, 2H). Exchangeable protons not observed.

### Example 70: Synthesis of (2-(4′-Fluoro-2′-(4-methyl-4H-1,2,4-triazol-3-yl)-[1,1′-biphenyl]-3-yl)-1H-benzo[d]imidazol-5-yl)methanol (BW-3)

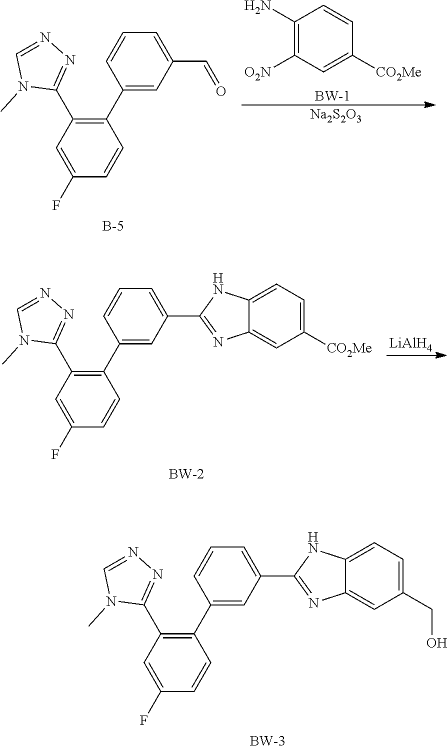

Step 1: Methyl 2-(4′-fluoro-2′-(4-methyl-4H-1,2,4-triazol-3-yl)-[1,1′-biphenyl]-3-yl)-1H-benzo[d]imidazole-6-carboxylate (BW-2). To a stirred solution of intermediate (B-5) (115 mg, 1 Eq, 409 μmol) in MeOH (1 mL) was added a solution of methyl 4-amino-3-nitrobenzoate (BW-1) (80.2 mg, 1 Eq, 409 μmol) and sodium dithionite (712 mg, 4.09 mL, 1 molar, 10 Eq, 4.09 mmol) at rt. The resulting mixture was stirred for 1 h at 65° C. and at rt 16 h. The reaction mixture was diluted with EtOAc (25 mL) and transferred into a separating funnel. The layer was washed with sat. aq. sol. of NH₄Cl (1×5 mL), with sat. aq. sol. of NaHCO₃ (1×5 mL) and brine. The combined organic layers were collected, dried (MgSO₄), filtered and concentrated in vacuo. The crude product was purified by chromatography on silica gel (24 g cartridge, 0-50% MeOH/DCM) to afford the sub-title compound (BW-2) (124 mg, 0.25 mmol, 60%, 85% Purity) as a sticky white solid. m/z 428.2 (M+H)⁺ (ES+). ¹H NMR (400 MHz, Methanol-d4) δ 8.36 (s, 1H), 8.27 (s, 1H), 8.09 (d, J=1.3 Hz, 2H), 7.97 (t, J=7.6 Hz, 1H), 7.79 (dd, J=8.6, 5.5 Hz, 1H), 7.58-7.52 (m, 2H), 7.51-7.42 (m, 2H), 7.22 (dt, J=7.8, 1.4 Hz, 1H), 3.35 (s, 3H), 3.19 (s, 3H). Exchangeable proton not visible.

Step 2: (2-(4′-Fluoro-2′-(4-methyl-4H-1,2,4-triazol-3-yl)-[1,1′-biphenyl]-3-yl)-1H-benzo[d]imidazol-5-yl)methanol (BW-3). To a solution of the product from step 1 above (BW-2) (120 mg, 80% Wt, 1 Eq, 225 μmol) in THF (2 mL) was added LiAlH₄ (17.0 mg, 449 μL, 1 molar, 2.0 Eq, 449 μmol) dropwise at −20° C. This mixture was stirred for 1 h at this temperature. An additional 1 eq. of LiAlH₄ was added at −20° C. and the reaction mixture was warned up to rt and stirred for 2 h at this temperature. The mixture was quenched with H₂O (0.2 mL), and NaOH (aq. 15%, 0.2 mL), and H₂O (0.6 mL) was added at 0° C. MgSO₄ was added and the mixture was warmed up to rt. The mixture was filtered over a silica plug and the solvent was removed in vacuo. The crude product was purified by chromatography on silica gel (24 g cartridge, 0-30% MeOH/DCM) to afford the title compound (BW-3) (64 mg, 0.16 mmol, 71%, 99% Purity). m/z 400.2 (M+H)⁺ (ES+). ¹H NMR (400 MHz, Methanol-d4) δ 8.36 (s, 1H), 8.10-8.00 (m, 2H), 7.78 (dd, J=8.7, 5.4 Hz, 1H), 7.61 (s, 2H), 7.54 (td, J=8.5, 2.8 Hz, 1H), 7.49-7.42 (m, 2H), 7.29 (d, J=8.3 Hz, 1H), 7.20-7.12 (m, 1H), 4.73 (s, 2H), 3.35 (s, 1H), 3.17 (s, 3H). One Exchangeable proton not visible.

### Example 71: Synthesis of 1-(((2-(4′-Fluoro-2′-(4-methyl-4H-1,2,4-triazol-3-yl)-[1,1′-biphenyl]-3-yl)-1H-benzo[d]imidazol-5-yl)methyl)amino)-2-methylpropan-2-ol (BX-2)

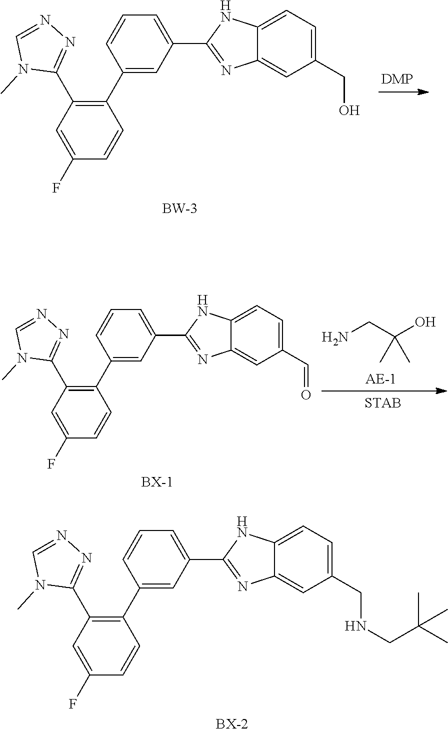

Step 1: 2-(4′-Fluoro-2′-(4-methyl-4H-1,2,4-triazol-3-yl)-[1,1′-biphenyl]-3-yl)-1H-benzo[d]imidazole-5-carbaldehyde (BX-1). Compound (BW-3) (50 mg, 1 Eq, 0.13 mmol) and DMP (0.11 g, 2.0 Eq, 0.25 mmol) were stirred in CHCl₃ (5 mL) at 45° C. for 3 h. The reaction mixture was diluted with EtOAc (20 mL) and washed with sat. aq. NaHCO₃ (10 mL) and brine (10 mL). Organics were separated, dried (MgSO₄), filtered and evaporated to afford the sub-title compound (BX-1) (81 mg, 0.12 mmol, 98%, 60% Purity) as white solid, which was used crude in the next step without purification. m/z 398.2 (M+H)⁺ (ES+).

Step 2: 1-(((2-(4′-Fluoro-2′-(4-methyl-4H-1,2,4-triazol-3-yl)-[1,1′-biphenyl]-3-yl)-1H-benzo[d]imidazol-5-yl)methyl)amino)-2-methylpropan-2-ol (BX-2). A solution of the product from step 1 above (BX-1) (25 mg, 90% Wt, 1 Eq, 57 μmol) and 1-amino-2-methylpropan-2-ol (AE-1) (10 mg, 11 μL, 2.0 Eq, 0.11 mmol) in CHCl₃ (1 mL) was stirred for 1 h at 40° C., then NaBH(OAc)₃ (36 mg, 3.0 Eq, 0.17 mmol) was added. The reaction mixture was stirred at 40° C. overnight. An additional 1 eq. of NaBH(OAc)₃ was added and the mixture was stirred for 3 h at rt. The reaction mixture was diluted with EtOAc (25 mL) and transferred into a separating funnel. The layer was washed with sat. aq. sol. of NaHCO₃ (2×5 mL) and brine. The combined organic layers were collected, dried (MgSO₄), filtered and concentrated in vacuo. The crude product was purified by chromatography on silica gel (12 g cartridge, 0-100% MeOH/DCM) to afford the title compound (BX-2) (10 mg, 21 μmol, 37%, 99% Purity) as a clear white solid. m/z 471.3 (M+H)⁺ (ES+). ¹H NMR (400 MHz, Methanol-d4) δ 8.39 (s, 1H), 8.11-8.05 (m, 2H), 7.81 (dd, J=8.6, 5.5 Hz, 1H), 7.64-7.53 (m, 3H), 7.51-7.44 (m, 2H), 7.32 (dd, J=8.3, 1.6 Hz, 1H), 7.19 (dt, J=7.9, 1.3 Hz, 1H), 3.95 (s, 2H), 3.20 (s, 3H), 2.61 (s, 2H), 1.25 (s, 6H). Exchangeable protons not visible.

### Example 72: Synthesis of (1S,2R)-2-(((2-(4′-Fluoro-2′-(4-methyl-4H-1,2,4-triazol-3-yl)-[1,1′-biphenyl]-3-yl)-1H-benzo[d]imidazol-5-yl)methyl)amino)cyclopentan-1-ol (BY-1)

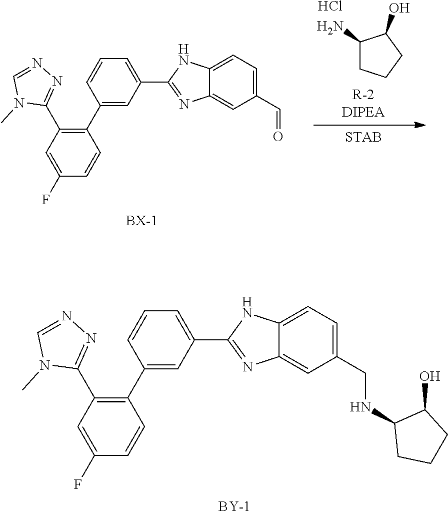

A solution of intermediate (BX-1) (25 mg, 90% Wt, 1 Eq, 57 μmol), (1S,2R)-2-aminocyclopentan-1-ol, HCl (R-2) (16 mg, 2.0 Eq, 0.11 mmol) and DIPEA (22 mg, 30 μL, 3.0 Eq, 0.17 mmol) in CHCl₃ (1 mL) was stirred for 1 h at 40° C., then NaBH(OAc)₃ (36 mg, 3.0 Eq, 0.17 mmol) was added. The reaction mixture was stirred at 40° C. overnight. An additional 1 eq. of NaBH(OAc)₃ was added and the mixture stirred for 3 h at rt. The reaction mixture was diluted with EtOAc (25 mL) and transferred into a separating funnel. The layer was washed with sat. aq. sol. of NaHCO₃ (2×5 mL) and brine. The combined organic layers were collected, dried (MgSO₄), filtered and concentrated in vacuo. The crude product was purified by chromatography on silica gel (12 g cartridge, 0-100% (0.7 M Ammonia/MeOH)/DCM) to afford the title compound (BY-1) (8 mg, 0.02 mmol, 30%, 94% Purity) as a clear white solid. m/z 483.4 (M+H)⁺ (ES+). ¹H NMR (400 MHz, Methanol-d4) δ 8.35 (s, 1H), 8.11 (s, 1H), 8.06 (dt, J=7.9, 1.3 Hz, 1H), 7.77 (dd, J=8.7, 5.4 Hz, 1H), 7.62-7.48 (m, 3H), 7.47-7.36 (m, 2H), 7.25 (d, J=8.3 Hz, 1H), 7.09 (d, J=7.7 Hz, 1H), 4.19-4.08 (m, 1H), 3.94 (d, J=12.6 Hz, 1H), 3.85 (d, J=12.5 Hz, 1H), 3.14 (s, 3H), 2.95 (td, J=8.3, 7.3, 4.1 Hz, 1H), 1.96-1.64 (m, 4H), 1.51 (dq, J=17.9, 9.4 Hz, 2H). Exchangeable protons not visible.

### Example 73: Synthesis of 3′-(5-(Hydroxymethyl)-7-(trifluoromethyl)-1H-benzo[d]imidazol-2-yl)-2-(4-methyl-4H-1,2,4-triazol-3-yl)-[1,1′-biphenyl]-4-carbonitrile (BZ-1)

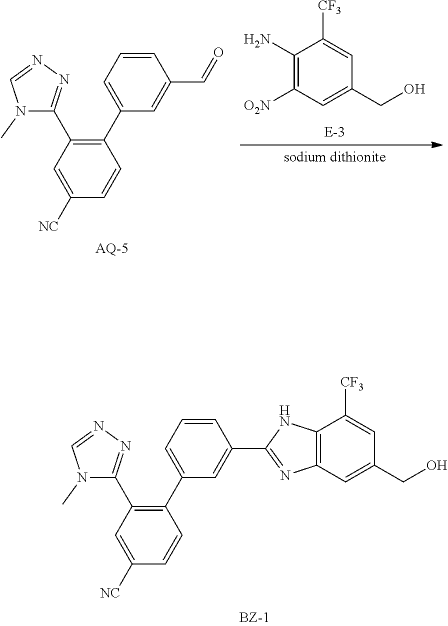

To a stirred solution of intermediate (AQ-5) (159 mg, 95% Wt, 1 Eq, 523 μmol) in MeOH (8 mL) was added a solution of 1M sodium dithionite (910 mg, 5.23 mL, 1 molar, 10 Eq, 5.23 mmol) and intermediate (E-3) (130 mg, 95% Wt, 1 Eq, 523 μmol) at rt. The resulting mixture was stirred for 1 h at 65° C. and at rt 36h. The reaction mixture was diluted with EtOAc (25 mL) and transferred into a separating funnel. The layer was washed with sat. aq. sol. of NH₄Cl (1×5 mL), with sat. aq. sol. of NaHCO₃ (1×5 mL) and brine. The combined organic layers were collected, dried (MgSO₄), filtered and concentrated in vacuo. The crude product was purified by chromatography on RP Flash C18 (24 g cartridge, 15-75% MeCN/10 mM ammonium bicarbonate) to afford the title compound (BZ-1) (123 mg, 0.26 mmol, 49%, 99% Purity) as a clear white solid. m/z 475.1 (M+H)⁺ (ES+). ¹H NMR (400 MHz, Methanol-d4) δ 8.43 (s, 1H), 8.26-8.13 (m, 3H), 8.10 (d, J=1.7 Hz, 1H), 8.01 (d, J=8.1 Hz, 1H), 7.86 (s, 1H), 7.62 (s, 1H), 7.55 (t, J=7.8 Hz, 1H), 7.27 (d, J=7.8 Hz, 1H), 4.81 (s, 2H), 3.24 (s, 3H). Exchangeable protons not visible.

### Example 74: Synthesis of 2-(4′-Fluoro-2′-(4-methyl-4H-1,2,4-triazol-3-yl)-[1,1′-biphenyl]-3-yl)-1H-benzo[d]imidazole (CA-2)

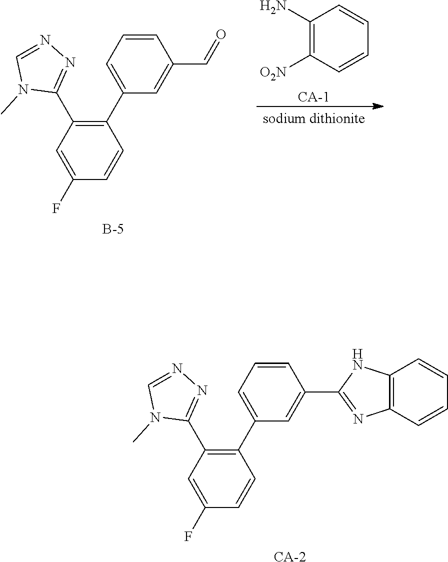

To a stirred solution of intermediate (B-5) (31.0 mg, 1 Eq, 110 μmol) in MeOH (1 mL) was added a solution of 2-nitroaniline (CA-1) (15.2 mg, 1 Eq, 110 μmol) and sodium dithionite (192 mg, 1.10 mL, 1 M, 10 Eq, 1.10 mmol) at rt. The resulting mixture was stirred for 1 h at 65° C. and at rt 16 h. The reaction mixture was diluted with EtOAc (25 mL) and transferred into a separating funnel. The layer was washed with sat. aq. sol. of NH₄Cl (5 mL), sat. aq. sol. of NaHCO₃ (5 mL) and brine. The combined organic layers were collected, dried (MgSO₄), filtered and concentrated in vacuo. The crude product was purified by preparative HPLC (Waters, Basic (0.3% ammonia), Basic, Waters X-Bridge BEH C18 ODB, 5 μm, 30×100 mm column, 15-100% MeCN in Water) to afford the title compound (CA-2) (26 mg, 70 μmol, 63%, 99% Purity) as a white solid. m/z 370.2 (M+H)⁺ (ES+). ¹H NMR (400 MHz, Methanol-d4) δ 8.39 (s, 1H), 8.08 (d, J=8.8 Hz, 2H), 7.82 (dd, J=8.7, 5.5 Hz, 1H), 7.64 (s, 2H), 7.60-7.54 (m, 1H), 7.53-7.44 (m, 2H), 7.31 (dd, J=6.0, 3.2 Hz, 2H), 7.21 (d, J=7.8 Hz, 1H), 3.21 (s, 3H). Exchangeable proton not visible.

### Example 75: Synthesis of 2-(4-Methyl-4H-1,2,4-triazol-3-yl)-3′-(4-(trifluoromethyl)-1H-benzo[d]imidazol-2-yl)-[1,1′-biphenyl]-4-carbonitrile (CB-1)

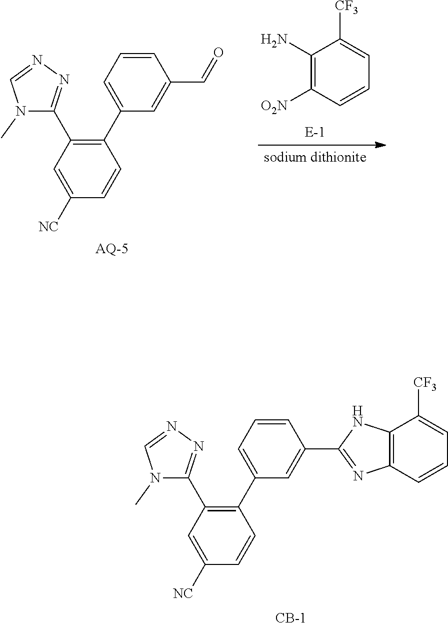

To a stirred solution of intermediate (AQ-5) (30.0 mg, 1 Eq, 104 μmol) in MeOH (1 mL) was added a solution of 2-nitro-6-(trifluoromethyl)aniline (E-1) (21.4 mg, 1 Eq, 104 μmol) and sodium dithionite (181 mg, 1.04 mL, 1 molar, 10 Eq, 1.04 mmol) at rt. The resulting mixture was stirred for 1 h at 65° C. and at rt 16 h. The reaction mixture was diluted with EtOAc (25 mL) and transferred into a separating funnel. The layer was washed with sat. aq. sol. of NH₄C1 (1×5 mL), sat. aq. sol. of NaHCO₃ (1×5 mL) and brine. The combined organic layers were collected, dried (MgSO₄), filtered and concentrated in vacuo. The crude product was purified by chromatography on silica gel (12 g cartridge, 0-50% MeOH/DCM) to afford the title compound (CB-1) (22 mg, 49 μmol, 47%, 99% Purity) as a pale-yellow solid. m/z 445.1 (M+H)⁺ (ES+). ¹H NMR (400 MHz, Methanol-d4) δ 8.43 (s, 1H), 8.28-8.13 (m, 3H), 8.10 (d, J=1.7 Hz, 1H), 8.01 (d, J=8.1 Hz, 1H), 7.87 (s, 1H), 7.61 (d, J=7.6 Hz, 1H), 7.55 (t, J=7.8 Hz, 1H), 7.44 (t, J=7.9 Hz, 1H), 7.28 (d, J=7.9 Hz, 1H), 3.24 (s, 3H). Exchangeable proton not visible.

### Example 76: Synthesis of (S)-3-(((2-(4′-Fluoro-2′-(4-methyl-4H-1,2,4-triazol-3-yl)-[1,1′-biphenyl]-3-yl)-7-(trifluoromethyl)-1H-benzo[d]imidazol-5-yl)methyl)amino)-1-methylpiperidin-2-one, formic Acid (CC-2)

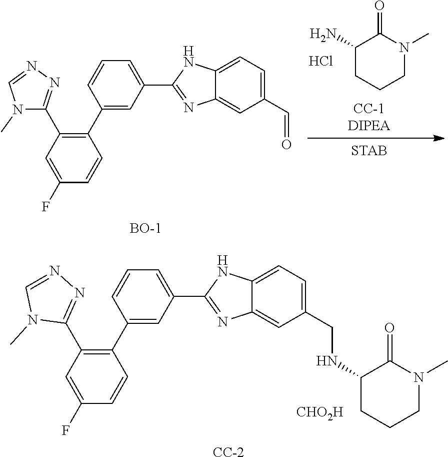

A solution of intermediate (BO-1) (30 mg, 90% Wt, 1 Eq, 58 μmol), (S)-3-amino-1-methylpiperidin-2-one, HCl (CC-1) (19 mg, 2.0 Eq, 0.12 mmol) and DIPEA (22 mg, 30 μL, 3.0 Eq, 0.17 mmol) in CHCl₃ (1 mL) was stirred 1 h at 40° C., then NaBH(OAc)₃ (61 mg, 5.0 Eq, 0.29 mmol) was added. The reaction mixture was stirred at 40° C. overnight. An additional 1 Eq. of NaBH(OAc)₃ was added and the mixture was stirred 5 h at 40° C. The reaction mixture was diluted with EtOAc (25 mL) and transferred into a separating funnel. The layer was washed with sat. aq. sol. of NaHCO₃ (2×5 mL) and brine. The combined organic layers were collected, dried (MgSO₄), filtered and concentrated in vacuo. The crude product was purified by preparative HPLC (Waters, Acidic (0.1% formic acid), Acidic, Waters X-Select CSH C18 ODB, 5 μm, 30×100 mm column, 5-100% MeCN in Water) to afford the title compound (CC-2) (6 mg, 0.01 mmol, 20%, 99% Purity) as a white solid. m/z 578.2 (M+H)⁺ (ES+). ¹H NMR (400 MHz, Methanol-d4) δ 8.45 (s, 1H), 8.35 (s, 1H), 8.16-8.06 (m, 2H), 7.89 (s, 1H), 7.80 (dd, J=8.6, 5.5 Hz, 1H), 7.66 (s, 1H), 7.53 (td, J=8.5, 2.8 Hz, 1H), 7.50-7.40 (m, 2H), 7.20 (d, J=7.8 Hz, 1H), 4.12 (s, 2H), 3.47-3.34 (m, 3H), 3.20 (s, 3H), 2.95 (s, 3H), 2.35-2.18 (m, 1H), 2.09-1.96 (m, 1H), 1.95-1.71 (m, 2H). Exchangeable protons not visible.

### Example 77: Synthesis of 2-Fluoro-N-((2-(4′-fluoro-2′-(4-methyl-4H-1,2,4-triazol-3-yl)-[1,1′-biphenyl]-3-yl)-7-(trifluoromethyl)-1H-benzo[d]imidazol-5-yl)methyl)-2-methylpropan-1-amine, Formic Acid (CD-2)

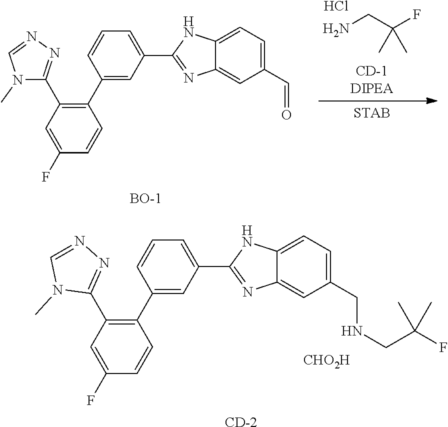

A solution of intermediate (BO-1) (30 mg, 90% Wt, 1 Eq, 58 μmol), 2-fluoro-2-methylpropan-1-amine, HCl (CD-1) (15 mg, 2.0 Eq, 0.12 mmol) and DIPEA (22 mg, 30 μL, 3.0 Eq, 0.17 mmol) in CHCl₃ (1 mL) was stirred 1 h at 40° C., then NaBH(OAc)₃ (61 mg, 5.0 Eq, 0.29 mmol) was added. The reaction mixture was stirred at 40° C. overnight. An additional 1 Eq. of NaBH(OAc)₃ was added and the mixture was stirred 5 h at 40° C. The reaction mixture was diluted with EtOAc (25 mL) and transferred into a separating funnel. The layer was washed with sat. aq. sol. of NaHCO₃ (2×5 mL) and brine. The combined organic layers were collected, dried (MgSO₄), filtered and concentrated in vacuo. The crude product was purified by preparative HPLC (Waters, Acidic (0.1% formic acid), Acidic, Waters X-Select CSH C18 ODB, 5 μm, 30×100 mm column, 5-100% MeCN in Water) to afford the title compound (CD-2) (5 mg, 8 μmol, 10%, 99% Purity) as a white solid. m/z 541.2 (M+H)⁺ (ES+). ¹H NMR (400 MHz, Methanol-d4) δ 8.54 (s, 1H), 8.39 (s, 1H), 8.21-8.09 (m, 2H), 7.96 (brs, 1H), 7.84 (dd, J=8.7, 5.5 Hz, 1H), 7.68 (s, 1H), 7.57 (td, J=8.5, 2.8 Hz, 1H), 7.53-7.44 (m, 2H), 7.24 (d, J=7.8 Hz, 1H), 4.11 (s, 2H), 3.23 (s, 3H), 2.86 (d, J=19.7 Hz, 2H), 1.43 (d, J=21.3 Hz, 6H). Exchangeable proton not visible.

### Example 78: Synthesis of 2-(1-Methyl-1H-pyrazol-5-yl)-5-(3-(3-((4-methyl-4H-1,2,4-triazol-3-yl)methyl)oxetan-3-yl)phenyl)-1,3,4-thiadiazole (CE-4)

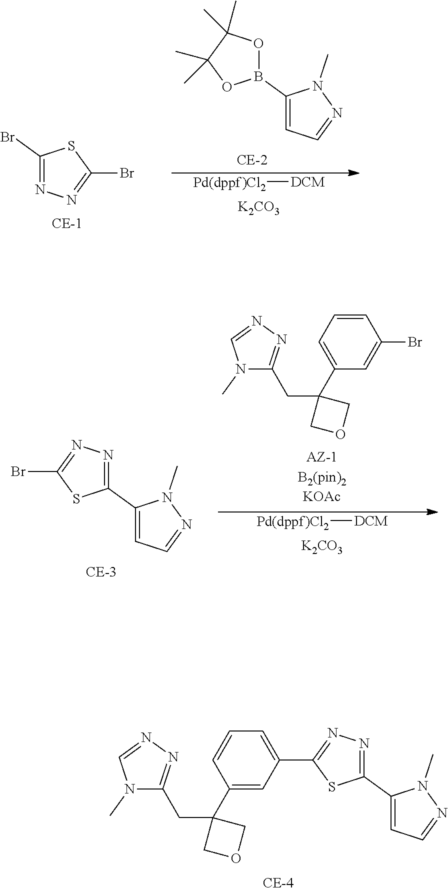

Step 1: 2-Bromo-5-(1-methyl-1H-pyrazol-5-yl)-1,3,4-thiadiazole (CE-3). To a solution of 1-methyl-5-(4,4,5,5-tetramethyl-1,3,2-dioxaborolan-2-yl)-1H-pyrazole (CE-2) (341 mg, 1 Eq, 1.64 mmol) and 2,5-dibromo-1,3,4-thiadiazole (CE-1) (400 mg, 1 Eq, 1.64 mmol) in degassed dioxane (3 mL)/water (0.75 mL) were successively added Pd(dppf)Cl₂·DCM (134 mg, 0.1 Eq, 164 μmol) and K₂CO₃ (680 mg, 3 Eq, 4.92 mmol). This mixture was stirred at 90° C. overnight. The reaction mixture was diluted with EtOAc (25 mL) and transferred into a separating funnel. The layer was washed with sat. aq. sol. of NH₄Cl (3×5 mL), sat. aq. sol. of NaHCO₃ (3×5 mL) and brine. The combined organic layers were collected, dried (MgSO₄), filtered and concentrated in vacuo. The reaction mixture was adsorbed onto silica gel and concentrated in vacuo. The crude product was purified by chromatography on silica gel (40 g cartridge, 0-20% EtOAc/isohexane) to afford the sub-title compound (CE-3) (27 mg, 0.11 mmol, 6.6%, 99% Purity) as a colourless solid. m/z 245.0 and 247.0 (M+H)⁺ (ES+). ¹H NMR (400 MHz, Chloroform-d) δ 7.55 (d, J=2.1 Hz, 1H), 6.65 (d, J=2.1 Hz, 1H), 4.30 (s, 3H).

Step 2: 2-(1-Methyl-1H-pyrazol-5-yl)-5-(3-(3-((4-methyl-4H-1,2,4-triazol-3-yl)methyl)oxetan-3-yl)phenyl)-1,3,4-thiadiazole (CE-4). To a solution of intermediate (AZ-1) (34 mg, 1 Eq, 0.11 mmol) and B₂(pin)₂ (31 mg, 1.1 Eq, 0.12 mmol) in degassed dioxane (1 mL) was successively added Pd(dppf)Cl₂·DCM (9.0 mg, 0.1 Eq, 11 μmol) and potassium acetate (32 mg, 3 Eq, 0.33 mmol). This mixture was stirred overnight at 100° C. for 2 h. The reaction mixture was cooled down to rt and a solution of the product from step 1 above (CE-3) (27 mg, 1 Eq, 0.11 mmol) in dioxane (1 mL) was added followed by a solution of K₂CO₃ (46 mg, 3 Eq, 0.33 mmol) in water (0.5 mL). The reaction mixture was stirred at 100° C. overnight. The reaction mixture was diluted with EtOAc (5 mL) and transferred into a separating funnel. The layer was washed with sat. aq. sol. of NH₄Cl (5 mL), sat. aq. sol. of NaHCO₃ (5 mL) and with brine (5 mL). The combined organic layers were collected, dried (MgSO₄), filtered and concentrated in vacuo. The crude product was purified by preparative HPLC (Waters basic method: Waters X-Select Prep-C18, 5 μm, 19×50 mm column, 5-95% MeCN in Water 0.1% (NH₄)₂CO₃) to afford the title compound (CE-4) (14 mg, 36 μmol, 32%, 100% Purity) as a clear white powder. m/z 394.4 (M+H)⁺ (ES+). ¹H NMR (400 MHz, Methanol-d4) δ 8.23 (s, 1H), 8.01-7.94 (m, 1H), 7.62 (d, J=2.1 Hz, 1H), 7.58-7.50 (m, 2H), 7.23-7.16 (m, 1H), 6.94 (d, J=2.1 Hz, 1H), 5.13 (q, J=6.3 Hz, 4H), 4.32 (s, 3H), 3.71 (s, 2H), 3.00 (s, 3H).

### Example 79: Synthesis of 1-Methyl-3-(3-(3-((4-methyl-4H-1,2,4-triazol-3-yl)methyl)oxetan-3-yl)phenyl)-5-(trifluoromethyl)pyridin-2(1H)-one (CF-2)

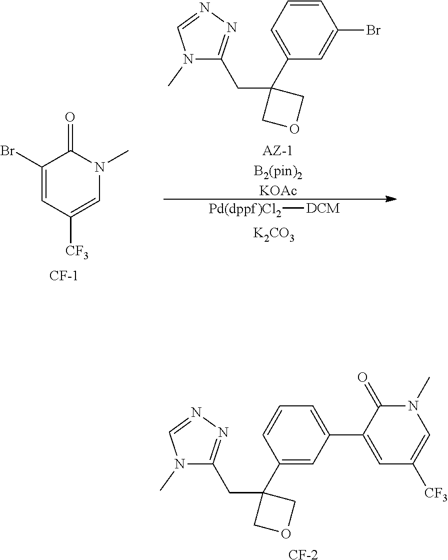

To a solution of intermediate (AZ-1) (35 mg, 1 Eq, 0.11 mmol) and B₂(pin)₂ (32 mg, 1.1 Eq, 0.12 mmol) in degassed dioxane (1 mL) was successively added Pd(dppf)Cl₂·DCM (9.3 mg, 0.1 Eq, 11 μmol) and potassium acetate (33 mg, 3 Eq, 0.34 mmol). This mixture was stirred at 100° C. for 1 h. The reaction mixture was cooled down to rt and a solution of 3-bromo-1-methyl-5-(trifluoromethyl)pyridin-2(1H)-one (CF-1) (29 mg, 1 Eq, 0.11 mmol) in dioxane (1 mL) was added followed by a solution of K₂CO₃ (47 mg, 3 Eq, 0.34 mmol) in water (0.5 mL). The reaction mixture was stirred at 100° C. 1 h. The reaction was cooled down to rt and the crude product was purified by chromatography on silica gel (4 g cartridge, 0-10% (0.7 M Ammonia/MeOH)/DCM) to afford the title compound (CF-2) (15 mg, 36 μmol, 32%, 98% Purity) as a tan solid. m/z 405.3 (M+H)⁺ (ES+). ¹H NMR (400 MHz, DMSO-d6) δ 8.43 (s, 1H), 8.17 (s, 1H), 7.68 (d, J=2.7 Hz, 1H), 7.57 (dt, J=8.1, 1.2 Hz, 1H), 7.29 (t, J=7.8 Hz, 1H), 7.23 (t, J=1.9 Hz, 1H), 6.86 (d, J=8.0 Hz, 1H), 4.94 (d, J=6.1 Hz, 2H), 4.89 (d, J=6.1 Hz, 2H), 3.56 (s, 3H), 3.50 (s, 2H), 2.87 (s, 3H).

### Example 80: Synthesis of 1-Methyl-3-(2′-(4-methyl-4H-1,2,4-triazol-3-yl)-[1,1′-biphenyl]-3-yl)-5-(trifluoromethyl)pyridin-2(1H)-one (CG-4)

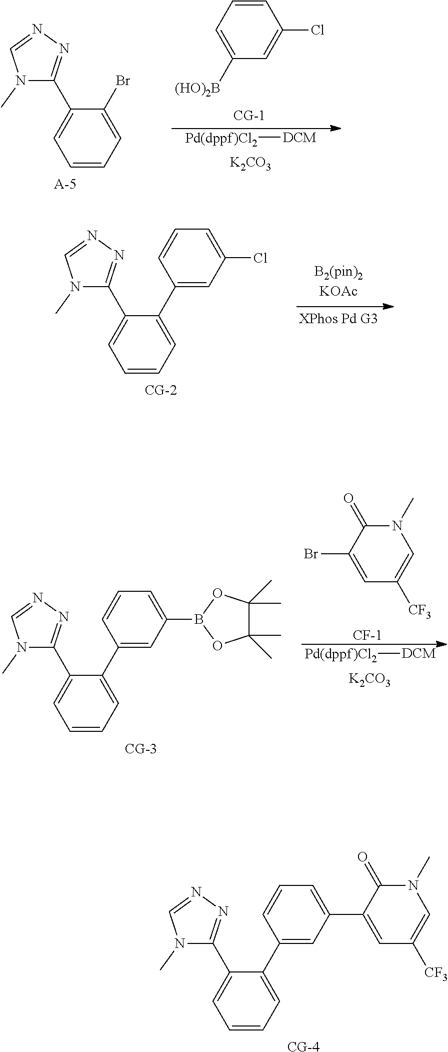

Step 1: 3-(3′-Chloro-[1,1′-biphenyl]-2-yl)-4-methyl-4H-1,2,4-triazole (CG-2). To a solution of intermediate (A-5) (1.2 g, 98% Wt, 1 Eq, 4.9 mmol) and (3-chlorophenyl)boronic acid (CG-1) (0.85 g, 1.1 Eq, 5.4 mmol) in dioxane (18 mL) and water (6 mL) were added Pd(dppf)Cl₂·DCM (0.20 g, 0.05 Eq, 0.25 mmol) and K₂CO₃ (2.0 g, 3 Eq, 15 mmol). The reaction mixture was degassed with nitrogen three times and heated overnight at 80° C. Additional (3-chlorophenyl)boronic acid (CG-1) (0.85 g, 1.1 Eq, 5.4 mmol), and Pd(dppf)Cl₂·DCM (0.20 g, 0.05 Eq, 0.25 mmol) were added and allowed to stir for a further 5 h. The reaction mixture was cooled to rt, diluted with EtOAc (50 mL) and washed with water (2×30 mL) and brine (30 mL). The organic layer was dried (MgSO₄), filtered and concentrated in vacuo. The crude product was purified by chromatography on silica gel (40 g cartridge, 0-100% hexane/EtOAc:EtOH (3:1)) to afford the sub-title compound (CG-2) (627.7 mg, 2.1 mmol, 42%, 90% Purity) as a dark brown tar. ¹H NMR (400 MHz, DMSO-d6) δ 8.43 (s, 1H), 7.72-7.67 (m, 1H), 7.64-7.50 (m, 3H), 7.40-7.31 (m, 2H), 7.18 (t, J=1.8 Hz, 1H), 7.05 (dt, J=7.4, 1.5 Hz, 1H), 3.07 (s, 3H).

Step 2: 4-Methyl-3-(3′-(4,4,5,5-tetramethyl-1,3,2-dioxaborolan-2-yl)-[1,1′-biphenyl]-2-yl)-4H-1,2,4-triazole (CG-3). A mixture of the product from step 1 above (CG-2) (526.6 mg, 90% Wt, 1 Eq, 1.757 mmol), XPhos Pd G3 (223.1 mg, 0.15 Eq, 263.6 μmol), B₂(pin)₂ (669.3 mg, 1.5 Eq, 2.636 mmol), and potassium acetate (517.3 mg, 3 Eq, 5.271 mmol) were dissolved in dioxane (25 mL), purged with nitrogen and heated to 80° C. overnight. Additional XPhos Pd G3 (223.1 mg, 0.15 Eq, 263.6 μmol), B₂(pin)₂ (669.3 mg, 1.5 Eq, 2.636 mmol) and potassium acetate (517.3 mg, 3 Eq, 5.271 mmol) were added. The reaction was purged with N₂ and heated to 95° C. for 5 h. The reaction mixture was cooled to rt, diluted with EtOAc (50 mL) washed with water (2×50 mL) and brine (2×50 mL). The organic extracts were combined, dried (MgSO₄) and concentrated in vacuo. The crude product was purified by chromatography on silica gel (40 g cartridge, 0-10% MeOH/DCM) to afford the sub-title compound (CG-3) (526.4 mg, 0.95 mmol, 54%, 65% Purity) as a sticky brown oil. m/z 362.4 (M+H)⁺ (ES+). ¹H NMR (400 MHz, DMSO-d6) δ 8.36 (s, 1H), 7.71-7.65 (m, 1H), 7.62-7.54 (m, 4H), 7.50-7.47 (m, 1H), 7.29 (t, J=7.5 Hz, 1H), 7.18-7.12 (m, 1H), 2.97 (s, 3H), 1.28 (s, 12H).

Step 3: 1-Methyl-3-(2′-(4-methyl-4H-1,2,4-triazol-3-yl)-[1,1′-biphenyl]-3-yl)-5-(trifluoromethyl)pyridin-2(1H)-one (CG-4). To a solution of the product from step 2 above (CG-3) (84.5 mg, 65% Wt, 1 Eq, 152 μmol) and 3-bromo-1-methyl-5-(trifluoromethyl)pyridin-2(1H)-one (CF-1) (38.9 mg, 1 Eq, 152 μmol) in degassed dioxane (2 mL) was successively added Pd(dppf)Cl₂·DCM (12.4 mg, 0.1 Eq, 15.2 μmol) and K₂CO₃ (63.0 mg, 3 Eq, 456 μmol) and water (1 mL). This mixture was stirred at 90° C. for 1 h. The crude product was purified by chromatography on silica gel (12 g cartridge, 0-10% MeOH/DCM) to afford desired product with pinacol impurity present. Material was triturated with Et₂O to afford the title compound (CG-4) (14.7 mg, 34 μmol, 22%, 95% Purity) as a grey solid. m/z 411.0 (M+H)⁺ (ES+). ¹H NMR (400 MHz, DMSO-d6) δ 8.44 (dd, J=2.7, 1.4 Hz, 1H), 8.39 (s, 1H), 7.72-7.64 (m, 4H), 7.61-7.54 (m, 2H), 7.48 (s, 1H), 7.33 (t, J=7.8 Hz, 1H), 7.05 (d, J=7.7 Hz, 1H), 3.56 (s, 3H), 3.01 (s, 3H).

### Example 81: Synthesis of Methyl 5-(2′-(4-methyl-4H-1,2,4-triazol-3-yl)-[1,1′-biphenyl]-3-yl)-6-oxo-3-(trifluoromethyl)-1,6-dihydropyridine-2-carboxylate (CH-2) and 5-(2′-(4-methyl-4H-1,2,4-triazol-3-yl)-[1,1′-biphenyl]-3-yl)-6-oxo-3-(trifluoromethyl)-1,6-dihydropyridine-2-carboxylic Acid (CH-3)

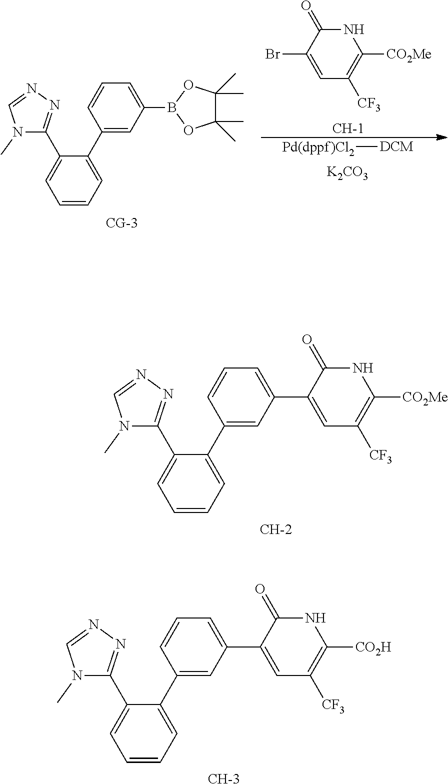

To a solution of intermediate (CG-3) (140 mg, 50% Wt, 1 Eq, 194 μmol) and methyl 5-bromo-6-oxo-3-(trifluoromethyl)-1,6-dihydropyridine-2-carboxylate (CH-1) (83.1 mg, 70% Wt, 1 Eq, 194 μmol) in degassed dioxane (1 mL) was successively added Pd(dppf)Cl₂·DCM (15.8 mg, 0.1 Eq, 19.4 μmol) and K₂CO₃ (80.3 mg, 3 Eq, 581 μmol) and water (0.5 mL). This mixture was stirred at 90° C. for 1 h. The reaction mixture was passed through a blue frit and the crude product was purified by chromatography on RP Flash C18 (24 g cartridge, 0-100% MeCN/10 mM ammonium bicarbonate) to afford a mixture of products which were separated by prep-HPLC, affording the Me-ester title compound (CH-2) (6.31 mg, 13 μmol, 6.8%, 95% Purity) as a brown solid. m/z 455.1 (M+H)⁺ (ES+). ¹H NMR (400 MHz, DMSO-d6) δ 12.88 (s, 1H), 8.38 (s, 1H), 7.69 (tt, J=6.6, 1.9 Hz, 4H), 7.61-7.52 (m, 2H), 7.47 (s, 1H), 7.38 (t, J=7.8 Hz, 1H), 7.15-7.07 (m, 1H), 3.91 (s, 3H), 3.04 (s, 3H). And the acid title compound (CH-3) (10.47 mg, 23 μmol, 12%, 95% Purity) as a white solid. m/z 440.9 (M+H)⁺ (ES+). ¹H NMR (400 MHz, DMSO-d6) δ 8.38 (s, 1H), 7.72-7.64 (m, 3H), 7.59 (s, 1H), 7.58-7.53 (m, 2H), 7.48 (s, 1H), 7.32 (t, J=7.8 Hz, 1H), 7.04 (d, J=7.5 Hz, 1H), 3.02 (s, 3H). Two exchangeable protons not observed.

### Example 82: Synthesis of 1-(2-(Dimethylamino)ethyl)-3-(2′-(4-methyl-4H-1,2,4-triazol-3-yl)-[1,1′-biphenyl]-3-yl)-5-(trifluoromethyl)pyridin-2(1H)-one (CI-4)

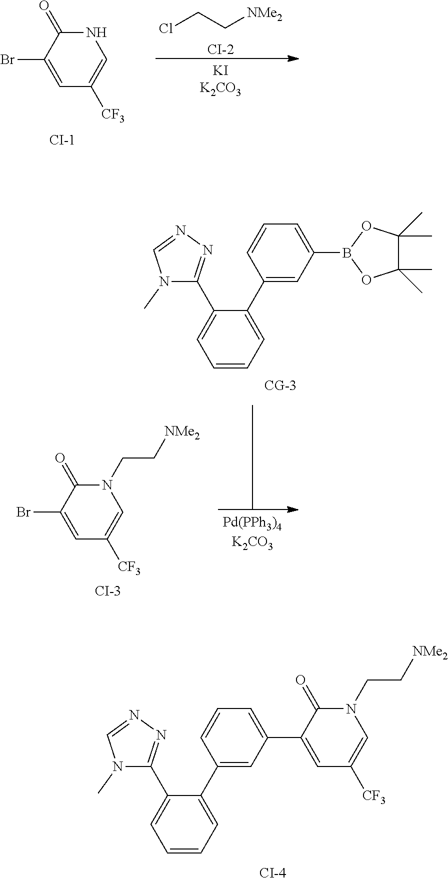

Step 1: 3-Bromo-1-(2-(dimethylamino)ethyl)-5-(trifluoromethyl)pyridin-2(1H)-one (CI-3). 3-bromo-5-(trifluoromethyl)pyridin-2(1H)-one (CI-1) (200 mg, 1 Eq, 826 μmol), 2-chloro-N,N-dimethylethan-1-amine, HCl (CI-2) (131 mg, 1.1 Eq, 909 μmol), KI (151 mg, 1.1 Eq, 909 μmol) and K₂CO₃ (251 mg, 2.2 Eq, 1.82 mmol) in DMF (4 mL) were stirred at 60° C. for 14 hour and then at rt over the weekend. The reaction temperature was elevated to 80° C. and stirring continued for 18 hours. The reaction mixture was allowed to cool, filtered and the filtrate loaded directly for purification by chromatography on RP Flash C18 (24 g cartridge, 0-100% MeCN/10 mM ammonium bicarbonate) to afford the sub-title compound (CI-3) (113 mg, 0.35 mmol, 43%, 98% Purity) as a yellow oil which solidified on standing to a waxy solid. m/z 313.0 & 315.0 (M+H)⁺ (ES+). ¹H NMR (400 MHz, DMSO-d6) δ 8.37 (dt, J=2.5, 1.2 Hz, 1H), 8.24 (d, J=2.5 Hz, 1H), 4.10 (t, J=6.2 Hz, 2H), 2.56-2.50 (m, 2H), 2.17 (s, 6H).

Step 2: 1-(2-(Dimethylamino)ethyl)-3-(2′-(4-methyl-4H-1,2,4-triazol-3-yl)-[1,1′-biphenyl]-3-yl)-5-(trifluoromethyl)pyridin-2(1H)-one (CI-4). Pd(PPh₃)₄(4.9 mg, 0.05 Eq, 4.3 μmol) was added to a mixture of intermediate (CG-3) (35 mg, 68% Wt, 1 Eq, 85 μmol), the product from step 1 above (CI-3) (38 mg, 98% Wt, 1.4 Eq, 0.12 mmol), K₂CO₃ (24 mg, 2.0 Eq, 0.17 mmol) and water (12 μL, 1 Eq, 85 μmol) in DME (1 mL). The reaction mixture was initially stirred 100° C. for 30 min (MW irradiation) and then twice further at 100° C./30 min each time; total time=90 min. The reaction mixture was allowed to stand at rt over the weekend. The crude reaction mixture was purified by prep-HPLC affording the title compound (CI-4) (7 mg, 0.01 mmol, 20%, 98% Purity). m/z 468.3 (M+H)⁺ (ES+). ¹H NMR (400 MHz, DMSO-d6) δ 8.38 (s, 1H), 8.34-8.31 (m, 1H), 7.73-7.64 (m, 4H), 7.59-7.54 (m, 2H), 7.48 (t, J=1.8 Hz, 1H), 7.33 (t, J=7.8 Hz, 1H), 7.08-7.03 (m, 1H), 4.12 (t, J=6.2 Hz, 2H), 3.02 (s, 3H), 2.55 (t, J=6.3 Hz, 2H), 2.19 (s, 6H).

### Example 83: Synthesis of 5-(1-{5-Azaspiro[2.4]heptan-5-yl}ethyl)-2-(3-{3-[(4-methyl-1,2,4-triazol-3-yl)methyl]oxetan-3-yl}phenyl)-7-(trifluoromethyl)-1,3-benzoxazole (AAB-9)

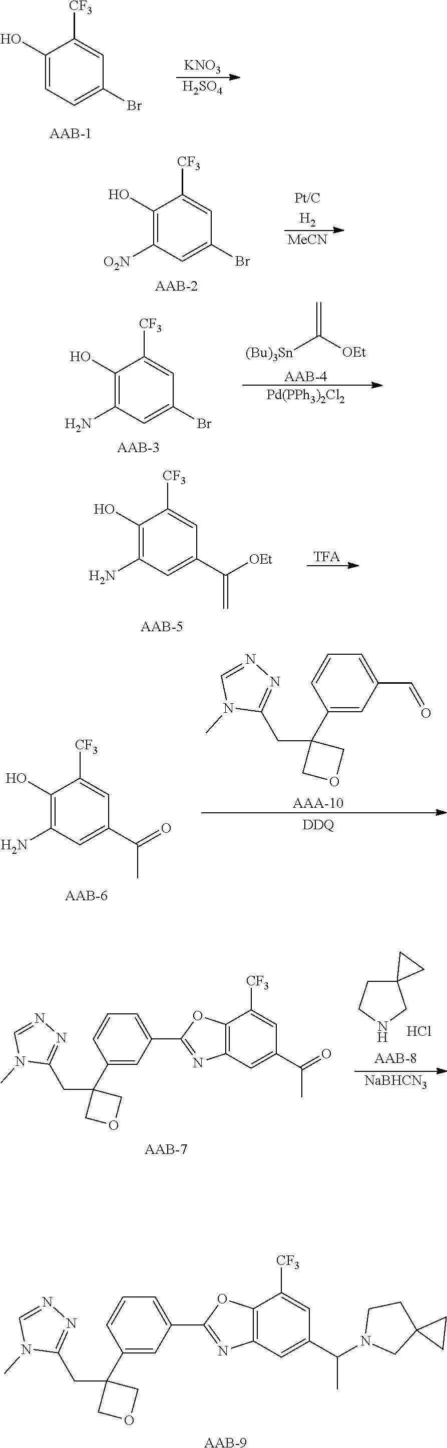

Step 1: 4-Bromo-2-nitro-6-(trifluoromethyl)phenol (AAB-2). Into a 100-mL round-bottom flask purged and maintained with an inert atmosphere of nitrogen, was placed 4-bromo-2-(trifluoromethyl)phenol (AAB-1) (2.00 g, 1 Eq, 8.30 mmol) in H₂SO₄ (20 mL), then nitric acid (1 mL) was dropwise added at 0° C. The resulting mixture was stirred for 45 min at 0° C. The reaction was then quenched by the addition of 30 mL of ice water at 0° C. The mixture was diluted with water and extracted with EtOAc (3×100 mL). The combined organic layers were washed with brine (2×100 mL), dried (Na₂SO₄). After filtration, the filtrate was concentrated in vacuo. The residue was purified by Prep-TLC with EtOAc/petroleum ether (5/1) to afford the sub-title compound (AAB-2) (1.3 g, 4.56 mmol, 58%) as a yellow solid. m/z 285.9 (M+H)⁺ (ES+).

Step 2: 2-Amino-4-bromo-6-(trifluoromethyl)phenol (AAB-3). Into a 250-mL round-bottom flask purged and maintained with an inert atmosphere of nitrogen, was placed the product from step 1 above (AAB-2) (1.00 g, 1 Eq, 3.50 mmol) in MeCN (40 mL), then platinum on carbon (100 mg, 5% Wt, 0.15 Eq, 513 μmol) was added at rt. The resulting mixture was stirred for 5 h at rt under nitrogen atmosphere. The resulting mixture was diluted with water and extracted with DCM (3×100 mL). The combined organic layers were washed with brine (2×100 mL), dried (Na₂SO₄). After filtration, the filtrate was concentrated in vacuo. The residue was purified by Prep-TLC with EtOAc/petroleum ether (10/1) to afford the sub-title compound (AAB-3) (800 mg, 3.13 mmol, 89%) as a yellow solid. m/z 256.0 (M+H)⁺ (ES+).

Step 3: 2-Amino-4-(1-ethoxyvinyl)-6-(trifluoromethyl)phenol (AAB-5). Into a 100-mL round-bottom flask purged and maintained with an inert atmosphere of nitrogen, was placed the product from step 2 above (AAB-3) (500 mg, 1 Eq, 1.95 mmol), dibutyl(1-ethoxyethenyl)propylstannane (AAB-4) (814 mg, 1.2 Eq, 2.34 mmol) and CsF (593 mg, 2 Eq, 3.91 mmol) in 1,4-dioxane (15 mL) at rt. Then Pd(PPh₃)₂Cl₂ (137 mg, 0.1 Eq, 195 μmol) was added at rt under nitrogen atmosphere. The resulting mixture was stirred for overnight at 80° C. under nitrogen atmosphere. The mixture was allowed to cool down to rt. The resulting mixture was diluted with water and extracted with DCM (3×80 mL). The combined organic layers were washed with brine (2×80 mL), dried (Na₂SO₄). After filtration, the filtrate was concentrated in vacuo. The crude product was purified by reverse flash column chromatography with the following conditions: Column, C18; mobile phase, Water (0.1% NH₄HCO₃) and MeCN (0% MeCN up to 50% in 10 min); Detector, UV 254/220 nm. This resulted in the sub-title compound (AAB-5) (200 mg, 810 μmol, 41%) as a yellow solid. m/z 248.1 (M+H)⁺ (ES+).

Step 4: 1-(3-Amino-4-hydroxy-5-(trifluoromethyl)phenyl)ethan-1-one (AAB-6). Into a 100-mL round-bottom flask purged and maintained with an inert atmosphere of nitrogen, was placed the product from step 3 above (AAB-5) (200 mg, 1 Eq, 809 μmol) in 1,4-dioxane (30 mL), then TFA (185 mg, 2 Eq, 1.62 mmol) was dropwise added at rt under nitrogen atmosphere. The resulting mixture was stirred for 3 h at rt. The resulting mixture was diluted with water and extracted with DCM (3×50 mL). The combined organic layers were washed with brine (2×50 mL), dried (Na₂SO₄). After filtration, the filtrate was concentrated in vacuo. The crude product was purified by reverse flash column chromatography with the following conditions: Column, C18; mobile phase, Water (0.1% NH₄HCO₃) and MeCN (0% MeCN up to 50% in 10 min); Detector, UV 254/220 nm to afford the sub-title compound (AAB-6) (150 mg, 685 μmol, 85%) as a white solid. m/z 220.1 (M+H)⁺ (ES+).

Step 5: 1-(2-(3-(3-((4-Methyl-4H-1,2,4-triazol-3-yl)methyl)oxetan-3-yl)phenyl)-7-(trifluoromethyl)benzo[d]oxazol-5-yl)ethan-1-one (AAB-7). Into a 50-mL round-bottom flask purged and maintained with an inert atmosphere of nitrogen, was placed the product from step 4 above (AAB-6) (130 mg, 1 Eq, 593 μmol) and intermediate (AAA-10) (153 mg, 1 Eq, 593 μmol) in DCM (17 mL), The resulting mixture was stirred for 1 h at 100° C. under nitrogen atmosphere. Then DDQ (270 mg, 2 Eq, 1.19 mmol) was added at rt under nitrogen atmosphere. The resulting mixture was stirred for 1 h at rt under nitrogen atmosphere. The resulting mixture was diluted with water and extracted with DCM (3×20 mL). The combined organic layers were washed with brine (2×20 mL), dried (Na₂SO₄). After filtration, the filtrate was concentrated in vacuo. The crude product was purified by reverse flash column chromatography with the following conditions: Column, C18; mobile phase, Water (0.1% NH₄HCO₃) and MeCN (0% MeCN up to 50% in 10 min); Detector, UV 254/220 nm. This resulted in the sub-title compound (AAB-7) (100 mg, 219 μmol, 37%) as a white solid. m/z 457.1 (M+H)⁺ (ES+).

Step 6: 5-(1-(5-Azaspiro[2.4]heptan-5-yl)ethyl)-2-(3-(3-((4-methyl-4H-1,2,4-triazol-3-yl)methyl)oxetan-3-yl)phenyl)-7-(trifluoromethyl)benzo[d]oxazole (AAB-9). Into a 40-mL vial purged and maintained with an inert atmosphere of nitrogen, was placed the product from step 5 above (AAB-7) (100 mg, 1 Eq, 219 μmol), 5-azaspiro[2.4]heptane (AAB-8) (26 mg, 1.2 Eq, 263 μmol) in DCM (16 mL). Then Ti(OiPr)₄ (125 mg, 438 μmol, 2 Eq) and NaBHCN₃ (28 mg, 2 Eq, 438 μmol) were added at rt under nitrogen atmosphere. The resulting mixture was stirred for 20 h at 40° C. under nitrogen atmosphere. The mixture was cooled to rt. The aqueous layer was extracted with DCM (3×30 mL). The combined organic layers were washed with brine (2×30 mL), dried (Na₂SO₄). After filtration, the filtrate was concentrated in vacuo. The crude product was purified by Prep-HPLC with the following conditions (Column: XBridge Prep OBD C18 Column, 30×150 mm, 5 μm; Mobile Phase A: Water (0.1% NH₄HCO₃), Mobile Phase B: MeCN; Flow rate: 60 mL/min; Gradient: 46% B to 63% B in 8 min; Wave Length: 254/220 nm; RT: 7.8) to afford the title compound (AAB-9) (50 mg, 93 μmol, 42%) as a white solid. m/z 538.5 (M+H)⁺ (ES+). ¹H NMR (400 MHz, DMSO-d6) δ 8.19 (d, J=2.8 Hz, 1H), 8.05 (d, J=7.6 Hz, 2H), 7.83 (d, J=2.7 Hz, 1H), 7.73 (s, 1H), 7.61-7.53 (m, 1H), 7.33 (d, J=8.0 Hz, 1H), 5.04-4.89 (m, 4H), 3.60 (d, J=2.7 Hz, 2H), 3.50 (d, J=6.7 Hz, 1H), 3.33 (s, 2H), 3.01 (d, J=2.7 Hz, 3H), 2.75 (d, J=8.0 Hz, 1H), 2.32 (m, 1H), 1.78-1.70 (m, 2H), 1.36 (m, 3H), 0.53-0.40 (m, 4H).

### Example 84: Synthesis of 6-(5-{5-Azaspiro[2.4]heptan-5-ylmethyl}-7-(trifluoromethyl)-1,3-benzoxazol-2-yl)-N-ethyl-4-[2-methyl-4-(4-methyl-1,2,4-triazol-3-yl)pyrazol-3-yl]pyridin-2-amine (AAC-5)

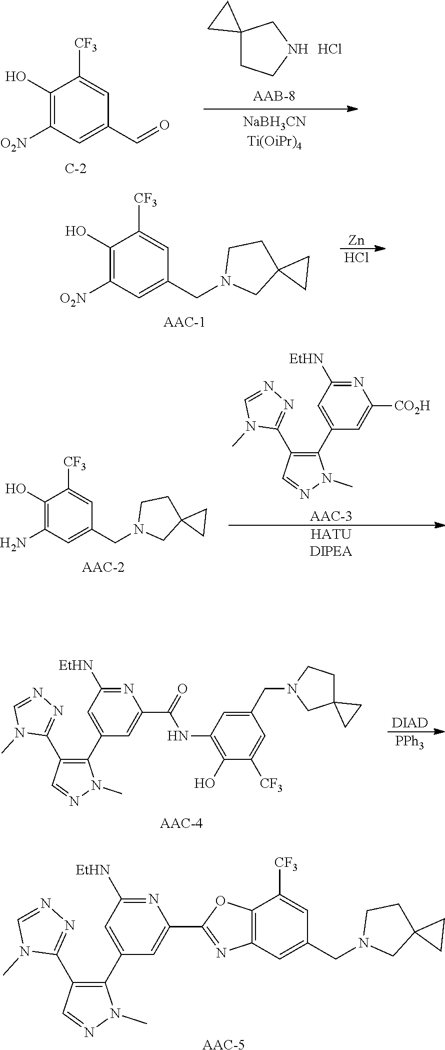

Step 1: 4-{5-azaspiro[2.4]heptan-5-ylmethyl}-2-nitro-6-(trifluoromethyl)phenol (AAC-1). To a stirred solution mixture of the intermediate (C-2) (500 mg, 1 Eq, 2.13 mmol) and 5-azaspiro[2.4]heptane, HCl (AAB-8) (341 mg, 1.2 Eq, 2.55 mmol) in DCM (30 mL) were added Ti(OiPr)₄ (1.2 g, 2 Eq, 4.25 mmol) and NaBHCN₃ (267 mg, 2 Eq, 4.25 mmol) at rt under nitrogen atmosphere. The resulting mixture was stirred for overnight at 40° C. under nitrogen atmosphere. The mixture was allowed to cool down to rt. The resulting mixture was diluted with water and extracted with EtOAc (3×80 mL). The combined organic layers were washed with brine (2×80 mL), dried (Na₂SO₄). After filtration, the filtrate was concentrated in vacuo. This resulted in the sub-title compound (AAC-1) (1.2 g, 4.0 mmol, crude) as a yellow solid. m/z 317.1 (M+H)⁺ (ES+).

Step 2: 2-Amino-4-{5-azaspiro[2.4]heptan-5-ylmethyl}-6-(trifluoromethyl)phenol (AAC-2). To a stirred solution of conc. HCl (4.5 mL) and water (4.5 mL) were added the product from step 1 above (AAC-1) (450 mg, 1 Eq, 1.42 mmol) and Fe powder (477 mg, 6 Eq, 8.54 mmol) at 0° C. The resulting mixture was stirred for 2 h at 0° C. The crude product was purified by reverse flash column chromatography with the following conditions: Column, C18; mobile phase, Water (0.1% FA) and MeCN (0% MeCN up to 10% in 20 min); Detector, UV 254/220 nm. This resulted in the sub-title compound (AAC-2) (200 mg, 699 μmol, 46%) as a brown oil. m/z 287.1 (M+H)⁺ (ES+).

Step 3: N-(5-{5-azaspiro[2.4]heptan-5-ylmethyl}-2-hydroxy-3-(trifluoromethyl)phenyl)-6-(ethylamino)-4-[2-methyl-4-(4-methyl-1,2,4-triazol-3-yl)pyrazol-3-yl]pyridine-2-carboxamide (AAC-4). To a stirred solution of 6-(ethylamino)-4-[2-methyl-4-(4-methyl-1,2,4-triazol-3-yl)pyrazol-3-yl]pyridine-2-carboxylic acid (AAC-3) (60 mg, 1 Eq, 183 μmol), DIPEA (118 mg, 5 Eq, 915 μmol) and HATU (139 mg, 2 Eq, 366 μmol) in THF (10 mL) at rt under nitrogen atmosphere. The resulting mixture was stirred for 20 min at rt under nitrogen atmosphere. To the above mixture was added the product from step 2 above (AAC-2) (63 mg, 1.2 Eq, 220 μmol) at rt. The resulting mixture was stirred for overnight at rt. The crude product was purified by reverse flash column chromatography with the following conditions: Column, C18; mobile phase, Water (0.1% NH₄HCO₃) and MeCN (0% MeCN up to 40% in 25 min); Detector, UV 254/220 nm. This resulted in the sub-title compound (AAC-4) (30 mg, 50.4 μmol, 25%) as a yellow solid. m/z 596.3 (M+H)⁺ (ES+).

Step 4: 6-(5-{5-Azaspiro[2.4]heptan-5-ylmethyl}-7-(trifluoromethyl)-1,3-benzoxazol-2-yl)-N-ethyl-4-[2-methyl-4-(4-methyl-1,2,4-triazol-3-yl)pyrazol-3-yl]pyridin-2-amine (AAC-5). To a stirred solution of triphenylphosphine (26 mg, 2 Eq, 100 μmol) and DIAD (20 mg, 2 Eq, 100 μmol) in THF (5 mL) was added the product from step 3 (AAC-4) (30 mg, 1 Eq, 50 μmol) in THF (0.5 mL) at rt under nitrogen atmosphere. The resulting mixture was stirred for 1 h at 60° C. under nitrogen atmosphere. The mixture was allowed to cool down to rt. The mixture was purified by Prep-HPLC with the following conditions (Column: Xselect CSH C18 OBD Column 30*150 mm 5m, n; Mobile Phase A: Water(0.1% FA), Mobile Phase B: MeCN; Flow rate: 60 mL/min; Gradient: 2% B to 15% B in 10 min, 15% B; Wave Length: 254/220 nm; RT: 9.32). This resulted in the title compound (AAC-5) (4.7 mg, 8.1 μmol, 16%) as a yellow solid. m/z 578.0 (M+H)⁺ (ES+). ¹H NMR (400 MHz, DMSO-d6) δ 8.48 (s, 1H), 8.06 (s, 1H), 7.95 (s, 1H), 7.75 (s, 1H), 7.40 (d, J=1.3 Hz, 1H), 7.23 (t, J=5.4 Hz, 1H), 6.72 (d, J=1.3 Hz, 1H), 3.91 (s, 3H), 3.79 (s, 2H), 3.51 (s, 3H), 3.39-3.31 (m, 2H), 2.70 (t, J=6.8 Hz, 2H), 2.46 (s, 2H), 1.76 (t, J=6.8 Hz, 2H), 1.19 (t, J=7.2 Hz, 3H), 0.54-0.47 (m, 4H).

### Example 85: Synthesis of 5-[(1R)-1-{5-azaspiro[2.4]heptan-5-yl}ethyl]-2-(3-{3-[(4-methyl-1,2,4-triazol-3-yl)methyl]oxetan-3-yl}phenyl)-7-(trifluorome thyl)-1,3-benzoxazole (AAD-1)

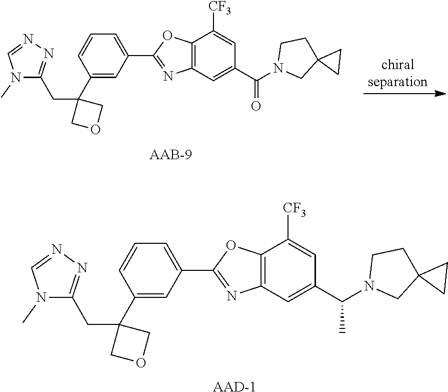 [SMILES: Cn1cnnc1CC1(c2cccc(-c3nc4cc(C(=O)N5CCC6(CC6)C5)cc(C(F)(F)F)c4o3)c2)COC1 ; "chiralseparation" ; C[C@H](c1cc(C(F)(F)F)c2oc(-c3cccc(C4(Cc5nncn5C)COC4)c3)nc2c1)N1CCC2(CC2)C1 ; "AAD-1"]

The crude product of intermediate (AAB-9) (50 mg, 1 Eq, 87 μmol) was separated by Chiral-Prep-HPLC with the following conditions: Column: CHIRALPAK IE-3, 4.6×50 mm, 3 um; Mobile Phase A: MtBE (0.1% DEA): MeOH=75:25; Flow rate: 1 mL/min; Gradient: 0% B to 0% B; Injection Volume: 5 ul. This resulted in the title compound (AAD-1) (16.8 mg, 31 μmol, 34%) as a white solid. m/z 538.5 (M+H)⁺ (ES+). ¹H NMR (400 MHz, DMSO-d6) δ 8.19 (s, 1H), 8.09-8.03 (m, 2H), 7.83 (t, J=1.8 Hz, 1H), 7.75 (s, 1H), 7.57 (t, J=7.8 Hz, 1H), 7.34 (d, J=7.6 Hz, 1H), 5.01 (d, J=6.1 Hz, 2H), 4.93 (d, J=6.2 Hz, 2H), 3.60 (s, 3H), 3.35 (s, 2H), 3.01 (s, 3H), 2.76 (s, 1H), 2.33 (s, 1H), 1.75 (s, 2H), 1.38 (s, 3H), 0.50 (d, J=9.1 Hz, 4H).

### Example 86: Synthesis of 5-[(1S)-1-{5-azaspiro[2.4]heptan-5-yl}ethyl]-2-(3-{3-[(4-methyl-1,2,4-triazol-3-yl)methyl]oxetan-3-yl}phenyl)-7-(trifluoromethyl)-1,3-benzoxazole (AAE-1)

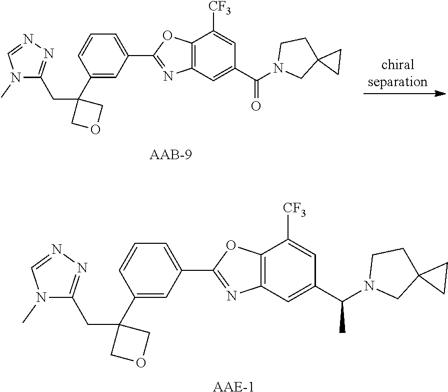 [SMILES: Cn1cnnc1CC1(c2cccc(-c3nc4cc(C(=O)N5CCC6(CC6)C5)cc(C(F)(F)F)c4o3)c2)COC1 ; "chiralseparation" ; C[C@@H](c1cc(C(F)(F)F)c2oc(-c3cccc(C4(Cc5nncn5C)COC4)c3)nc2c1)N1CCC2(CC2)C1 ; "AAE-1"]

The crude product of intermediate (AAB-9) (50 mg, 1 Eq, 87 μmol) was separated by Chiral-Prep-HPLC with the following conditions: Column: CHIRALPAK IE-3, 4.6×50 mm, 3 um; Mobile Phase A: MtBE (0.1% DEA): MeOH=75:25; Flow rate: 1 mL/min; Gradient: 0% B to 0% B; Injection Volume: 5 ul. This resulted in the title compound (AAE-1) (17.8 mg, 33 μmol, 36%) as a white solid. m/z 538.5 (M+H)⁺ (ES+). ¹H NMR (400 MHz, DMSO-d6) δ 8.19 (s, 1H), 8.09-8.03 (m, 2H), 7.83 (t, J=1.8 Hz, 1H), 7.75 (s, 1H), 7.57 (t, J=7.8 Hz, 1H), 7.34 (d, J=7.6 Hz, 1H), 5.01 (d, J=6.1 Hz, 2H), 4.93 (d, J=6.2 Hz, 2H), 3.60 (s, 3H), 3.35 (s, 2H), 3.01 (s, 3H), 2.76 (s, 1H), 2.33 (s, 1H), 1.75 (s, 2H), 1.38 (s, 3H), 0.50 (d, J=9.1 Hz, 4H).

### Example 87: Synthesis of 6-(6-{5-Azaspiro[2.4]heptan-5-ylmethyl}-4-(trifluorome thyl)-3H-1,3-benzodiazol-2-yl)-N-ethyl-4-[2-(4-methyl-1,2,4-triazol-3-yl)phenyl]pyridin-2-amine (AAG-4)

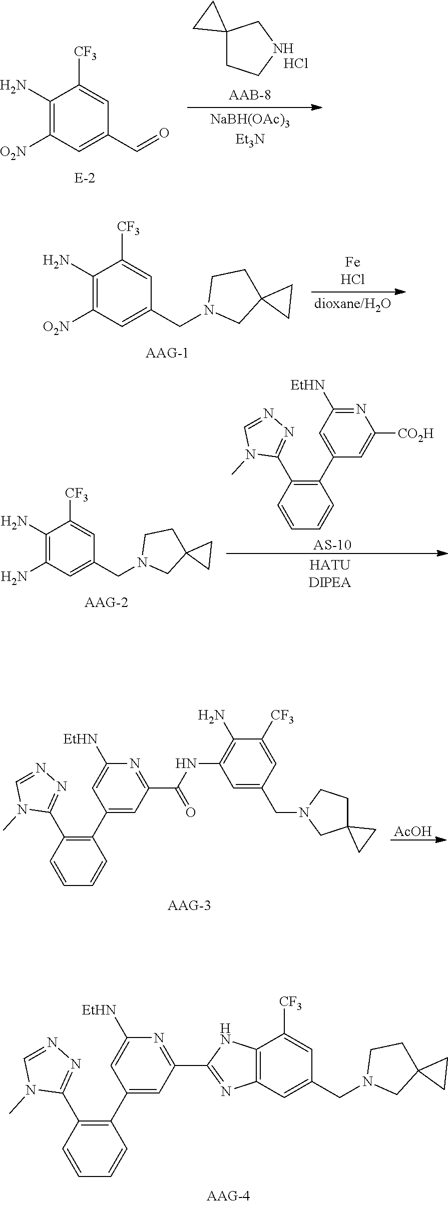

Step 1: 4-{5-Azaspiro[2.4]heptan-5-ylmethyl}-2-nitro-6-trifluoromethyl aniline (AAG-1). A solution of intermediate (AAB-8) (49 mg, 1.3 Eq, 499 μmol) in DCE (5 mL) was treated with Et₃N (50 mg, 1.3 Eq, 499 μmol) for 5 min at rt followed by the addition of intermediate (E-2) (90 mg, 1 Eq, 384 μmol) and NaBH(OAc)₃ (245 mg, 3 Eq, 1.15 mmol) in portions at rt. The resulting mixture was stirred for 5 h at rt. The resulting mixture was filtered; the filtrate was concentrated in vacuo. The residue was applied on a silica gel column chromatography with DCM/MeOH (12/1) to afford the sub-title compound (AAG-1) (100 mg, 317 μmol, 83%) as a yellow solid. m/z 316.1 (M+H)⁺ (ES+).

Step 2: 5-{5-Azaspiro[2.4]heptan-5-ylmethyl}-3-trifluoromethylbenzene-1,2-diamine (AAG-2). To a stirred solution of the product from step 1 above (AAG-1) (90 mg, 1 Eq, 285 mmol) in HCl in 1,4-dioxane (1 mL, 4 molar) and HCl (3 mL, 6 molar) was added Fe (93 mg, 5 Eq, 1.43 mmol) at 0° C. The resulting mixture was stirred for 2 h at rt. The resulting mixture was concentrated in vacuo. The crude product was purified by reverse flash column chromatography with the following conditions: Column, C18; mobile phase, Water (0.1% HCl) and MeCN (0% MeCN up to 15% in 15 min); Detector, UV 254/220 nm) to afford the sub-title compound (AAG-2) (50 mg, 175 μmol, 61%) as a yellow solid. m/z 286.1 (M+H)⁺ (ES+).

Step 3: N-(2-Amino-5-{5-azaspiro[2.4]heptan-5-ylmethyl}-3-trifluoromethylphenyl)-6-(ethylamino)-4-[2-(4-methyl-1,2,4-triazol-3-yl)phenyl]pyridine-2-carboxamide (AAG-3). A solution of intermediate (AS-10) (45 mg, 1 Eq, 14 μmol) in DMF (5 mL) was treated with DIPEA (54 mg, 3 Eq, 420 μmol) and HATU (59 mg, 1.1 Eq, 154 μmol) for 10 min at rt followed by the addition of the product from step 2 above (AAG-2) (40 mg, 1 Eq, 140 μmol) at rt. The resulting mixture was stirred for 2 h at rt. The resulting mixture was diluted with water and extracted with EtOAc (3×20 mL). The combined organic layers were washed with brine (2×20 mL), dried (Na₂SO₄). After filtration, the filtrate was concentrated in vacuo. The residue was purified by Prep-TLC with DCM/MeOH (7/1) to afford the sub-title compound (AAG-6) (52 mg, 87.3 μmol, 63%) as a yellow solid. m/z 596.3 (M+H)⁺ (ES+).

Step 4: 6-(6-{5-Azaspiro[2.4]heptan-5-ylmethyl}-4-(trifluoromethyl)-3H-1,3-benzodiazol-2-yl)-N-ethyl-4-[2-(4-methyl-1,2,4-triazol-3-yl)phenyl]pyridin-2-amine (AAG-4). A solution of the product from step 3 above (AAG-3) (52 mg, 1 Eq, 88 μmol) in acetic acid (5 mL) was stirred for 2 h at 80° C. under nitrogen atmosphere. The mixture was allowed to cool down to rt. The resulting mixture was concentrated in vacuo. The crude product was purified by Prep-HPLC with the following conditions (Column: XBridge Prep OBD C18 Column, 30*150 mm, 5 μm; Mobile Phase A: Water (0.1% NH₄HCO₃), Mobile Phase B: MeCN; Flow rate: 60 mL/min; Gradient: 42% B to 59% B in 10 min, 59% B; Wavelength: 254/220 nm; RT: 9.1) to afford the title compound (AAG-4) (22.1 mg, 38.6 μmol, 43%) as a light-yellow solid. m/z 573.1 (M+H)⁺ (ES+). ¹H NMR (400 MHz, DMSO-d6) δ 12.77 (s, 1H), 8.45 (s, 1H), 7.82-7.59 (m, 5H), 7.48 (s, 1H), 7.31 (d, J=1.4 Hz, 1H), 6.76 (t, J=5.5 Hz, 1H), 6.20 (d, J=1.4 Hz, 1H), 3.77 (s, 2H), 3.50-3.38 (m, 2H), 3.22 (s, 3H), 2.71 (t, J=6.7 Hz, 2H), 2.46 (s, 2H), 1.77 (t, J=6.8 Hz, 2H), 1.17 (t, J=7.2 Hz, 3H), 0.58-0.42 (m, 4H).

### Example 88: Synthesis of 2-[2′-(4-Methyl-1,2,4-triazol-3-yl)-[1,1′-biphenyl]-3-yl]-6-{[(3S)-3-methylpiperidin-1-yl]methyl}-4-(trifluoromethyl)-3H-1,3-benzodiazole (AAI-5)

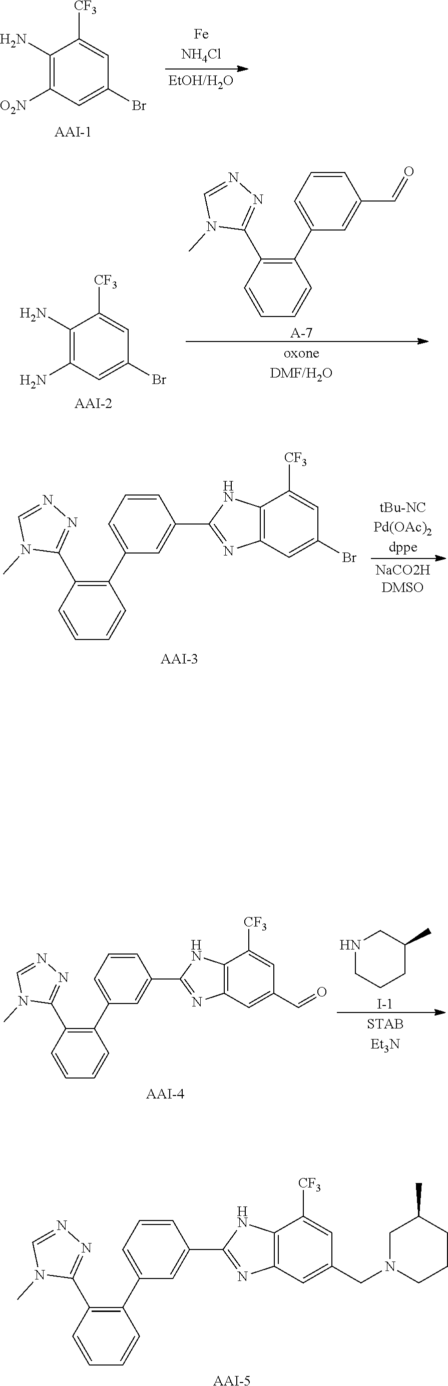

Step 1: 5-Bromo-3-(trifluoromethyl)benzene-1,2-diamine (AAI-2). To a stirred solution of 4-bromo-2-nitro-6-(trifluoromethyl) aniline (AAI-1) (285 mg, 1 Eq, 1.0 mmol) in EtOH (6 mL) and water (1.5 mL) was added Fe powder (279 mg, 5 Eq, 5.0 mmol) and NH₄Cl (535 mg, 10 Eq, 10.0 mmol) at rt. The resulting mixture was stirred for 2 h at 80° C. The mixture was allowed to cool down to rt and concentrated in vacuo. After filtration, the filtrate was concentrated in vacuo. The residue was purified by Prep-TLC with petroleum ether/EtOAc (3/1). This resulted in the sub-title compound (AAI-2) (240 mg, 945 μmol, 94%) as orange oil. m/z 255.0 (M+H)⁺ (ES+).

Step 2: 6-Bromo-2-[2′-(4-methyl-1,2,4-triazol-3-yl)-[1,1′-biphenyl]-3-yl]-4-(trifluoromethyl)-3H-1,3-benzodiazole (AAI-3). To a stirred solution of the product from step 1 above (AAI-2) (102 mg, 1 Eq, 400 μmol) and intermediate (A-7) (105 mg, 1 Eq, 400 μmol) in DMF (6 mL) and water (0.2 mL) were added oxone (67 mg, 1 Eq, 400 μmol) under a nitrogen atmosphere. After stirring for 3 h at rt, the crude product was purified by reverse flash column chromatography with the following conditions: Column, C18; mobile phase, Water (0.1% NH₄HCO₃) and MeCN (30% MeCN up to 60% in 10 min); Detector, UV 254/220 nm. The product-containing fractions were combined and concentrated in vacuo. This resulted in the sub-title compound (AAI-3) (150 mg, 302 μmol, 75%) as a yellow solid. m/z 498.0 (M+H)⁺ (ES+).

Step 3: 2-[2′-(4-Methyl-1,2,4-triazol-3-yl)-[1,1′-biphenyl]-3-yl]-7-(trifluoromethyl)-1H-1,3-benzodiazole-5-carbaldehyde (AAI-4). To a stirred solution of the product from step above (AAI-3) (110 mg, 1 Eq, 221 μmol) and tert-butyl isocyanide (22 mg, 1.2 Eq, 265 μmol) in DMSO (4 mL) was added sodium formate (30 mg, 2 Eq, 442 μmol), diphos (18 mg, 0.2 Eq, 44 μmol) and Pd(OAc)₂ (5 mg, 0.1 Eq, 22 μmol) at rt under nitrogen atmosphere. The resulting mixture was stirred overnight at 120° C. under nitrogen atmosphere. The mixture was cooled to rt and purified by reverse flash column chromatography with the following conditions: Column, C18; mobile phase, Water (0.1% NH₄HCO₃) and MeCN (30% MeCN up to 60% in 10 min); Detector, UV 254/220 nm. The product-containing fractions were combined and concentrated in vacuo. This resulted in the sub-title compound (AAI-4) (60 mg, 134 μmol, 61%) as a white solid. m/z 448.1 (M+H)⁺ (ES+).

Step 4: 2-[2′-(4-Methyl-1,2,4-triazol-3-yl)-[1,1′-biphenyl]-3-yl]-6-{[(3S)-3-methylpiperidin-1-yl]methyl}-4-(trifluoromethyl)-3H-1,3-benzodiazole (AAI-5). To a stirred solution of the product from step 3 above (AAI-4) (30 mg, 1 Eq, 67 μmol) and (S)-3-methylpiperidine (I-1) (13 mg, 2 Eq, 134 μmol) in DCM (4 mL) was added Et₃N (14 mg, 2 Eq, 134 μmol) and NaBH(OAc)₃ (28 mg, 2 Eq, 134 μmol) at rt. The resulting mixture was stirred additional overnight at 40° C. under nitrogen atmosphere. The mixture was cooled to rt and concentrated in vacuo. The crude product was purified by reverse flash column chromatography with the following conditions: Column, C18; mobile phase, Water (0.1% NH₄HCO₃) and MeCN (50% ACN up to 80% in 10 min); Detector, UV 254/220 nm. The product-containing fractions were combined and concentrated in vacuo. The crude product was purified by Prep-HPLC with the following conditions: Column: XBridge Prep OBD C18 Column, 30×150 mm 5 um; Mobile Phase A: Water (0.1% NH₄HCO₃), Mobile Phase B: MeCN; Flow rate: 60 mL/min; Gradient: 52 B to 62 B in 8 min; Detector, UV 254/210 nm; RT: 7.63. The product-containing fractions were combined and evaporated partially in vacuo and lyophilized overnight to afford the title compound (AAI-5) (13.1 mg, 24.7 μmol, 37%) as a white solid. m/z 531.4 (M+H)⁺ (ES+). ¹H NMR (400 MHz, Methanol-d4) δ 8.36 (s, 1H), 8.20-8.08 (m, 2H), 7.88-7.75 (m, 3H), 7.71-7.58 (m, 3H), 7.50 (t, J=7.8 Hz, 1H), 7.26 (d, J=7.8 Hz, 1H), 3.72 (s, 2H), 3.20 (s, 3H), 2.96-2.85 (m, 2H), 2.07-1.96 (m, 1H), 1.80-1.59 (m, 5H), 0.99-0.85 (m, 4H).

### Example 89: Synthesis of 2-[2′-(4-Methyl-1,2,4-triazol-3-yl)-[1,1′-biphenyl]-3-yl]-6-{[(3R)-3-methylpiperidin-1-yl]methyl}-4-(trifluoromethyl)-3H-1,3-benzodiazole (AAJ-1)

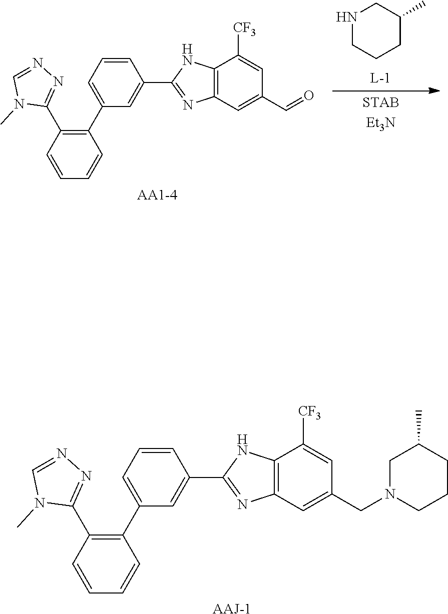

To a stirred solution of the intermediate (AAI-4) (30 mg, 1 Eq, 67 μmol) and (R)-3-methylpiperidine (L-1) (13 mg, 2 Eq, 134 μmol) in DCM (4 mL) was added Et₃N (14 mg, 2 Eq, 134 μmol) and NaBH(OAc)₃ (28 mg, 2 Eq, 134 μmol) at rt. The resulting mixture was stirred overnight at 40° C. under nitrogen atmosphere. The mixture was cooled to rt and concentrated in vacuo. The crude product was purified by reverse flash column chromatography with the following conditions: Column, C18; mobile phase, Water (0.1% NH₄HCO₃) and MeCN (50% MeCN up to 80% in 10 min); Detector, UV 254/220 nm. The product-containing fractions were combined and concentrated in vacuo. The crude product was purified by Prep-HPLC with the following conditions: Column: XBridge Prep OBD C18 Column, 30×150 mm 5 um; Mobile Phase A: Water (0.1% NH₄HCO₃), Mobile Phase B: MeCN; Flow rate: 60 mL/min; Gradient: 45 B to 60 B in 10 min; Detector, UV 254/210 nm; RT: 8.83. The product-containing fractions were combined and evaporated partially in vacuo and lyophilized overnight to afford the title compound (AAJ-1) (14.7 mg, 41.32%) as a white solid. m/z 531.2 (M+H)⁺ (ES+). ¹H NMR (400 MHz, Methanol-d4) δ 8.37 (s, 1H), 8.23-8.08 (m, 2H), 7.89-7.74 (m, 3H), 7.71-7.57 (m, 3H), 7.50 (t, J=7.8 Hz, 1H), 7.26 (d, J=7.7 Hz, 1H), 3.73 (s, 2H), 3.20 (s, 3H), 2.97-2.86 (m, 2H), 2.08-1.97 (m, 1H), 1.79-1.53 (m, 5H), 1.01-0.86 (m, 4H).

### Example 90: Synthesis of 5-Bromo-2-(3-(3-((4-methyl-4H-1,2,4-triazol-3-yl)methyl) oxetan-3-yl)phenyl)-7-(trifluoromethyl)benzo[d]oxazole (AAK-1)

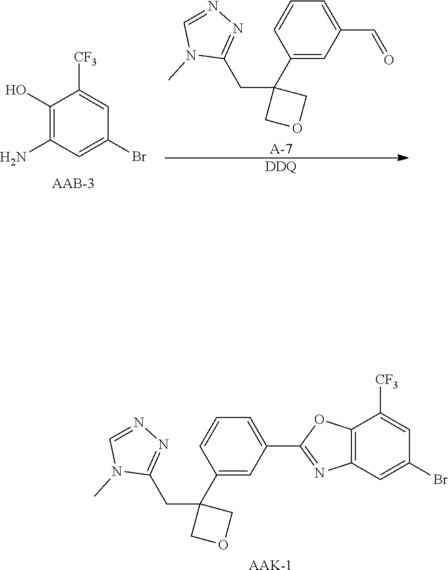

To a stirred mixture of intermediate (AAB-3) (30 mg, 1 Eq, 117 μmol) in DCM (2 mL) was added intermediate (A-7) (36 mg, 1.2 Eq, 140 μmol) and DDQ (2.66 mg, 0.1 Eq, 12 μmol) at rt under nitrogen atmosphere. The resulting mixture was stirred for 2 h at 60° C. under nitrogen atmosphere. The mixture was allowed to cool down to rt. The resulting mixture was diluted with water (5 mL). The resulting mixture was extracted with EtOAc (3×10 mL). The combined organic layers were washed with brine (2×5 mL), dried (Na₂SO₄). After filtration, the filtrate was concentrated in vacuo. The crude product was purified by Prep-HPLC with the following conditions: Column: Sunfire prep C18 column, 30*150 mm, 5 μm; Mobile Phase A: Water(0.1% FA), Mobile Phase B: MeCN; Flow rate: 60 mL/min; Gradient: 4% B to 34% B in 7 min; Detector, UV 254/220; RT: 6.02. This resulted in the title compound (AAK-1) (10.0 mg, 20 μmol, 17%) as a white solid. m/z 492.9/494.9 (M+H)⁺ (ES+). ¹H NMR (400 MHz, DMSO-d6) δ 8.44 (d, J=1.8 Hz, 1H), 8.20 (s, 1H), 8.09-8.03 (m, 1.3 Hz, 1H), 8.03-7.98 (m, 1H), 7.88 (t, J=1.8 Hz, 1H), 7.58 (t, J=7.8 Hz, 1H), 7.38-7.33 (m, 1H), 5.01 (d, J=6.2 Hz, 2H), 4.92 (d, J=6.2 Hz, 2H), 3.60 (s, 2H), 3.03 (s, 3H).

### Example 91: Synthesis of 5-((5-Azaspiro[2.4]heptan-5-yl)methyl)-2-(4′-fluoro-2′-(4-methyl-4H-1,2,4-triazol-3-yl)-[1,1′-biphenyl]-3-yl)-7-(trifluoromethyl) benzo[d]oxazole (AAL-5)

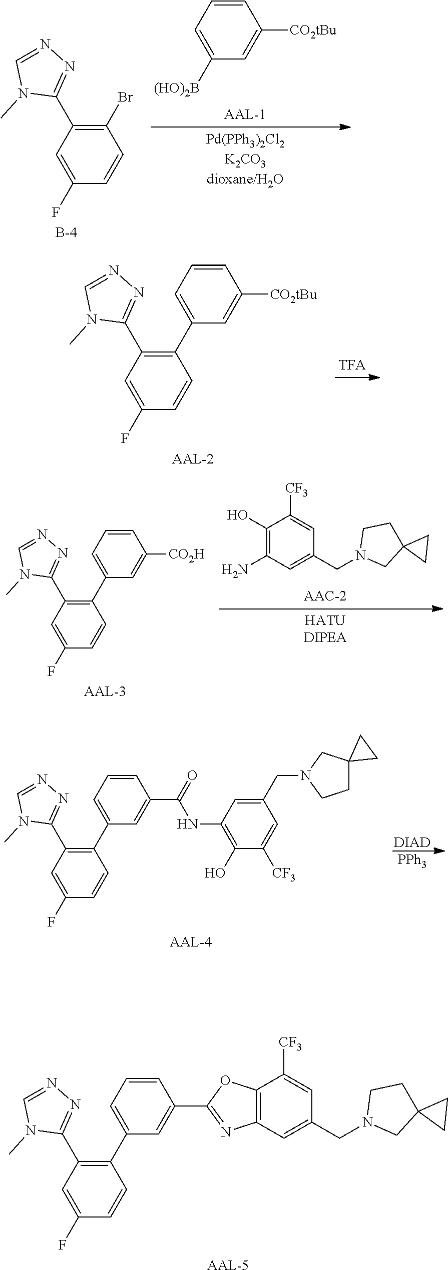

Step 1: tert-Butyl 4′-fluoro-2′-(4-methyl-1,2,4-triazol-3-yl)-[1,1′-biphenyl]-3-carboxylate (AAL-2). To a stirred solution of intermediate (B-4) (256 mg, 1 Eq, 1.00 mmol) and 3-(tert-butoxycarbonyl)-phenylboronic acid (AAL-1) (222 mg, 1 Eq, 1.00 mmol) in 1,4-dioxane (8 mL) and water (2 mL) were added K₂CO₃ (276 mg, 2 Eq, 2.00 mmol) and Pd(PPh₃)₂Cl₂ (70 mg, 0.1 Eq, 0.10 mmol) at rt under nitrogen atmosphere. The resulting mixture was stirred for 16 hours at 60° C. under nitrogen atmosphere. The resulting mixture was cooled to rt. The resulting mixture was concentrated in vacuo. The residue was purified by Prep-TLC with DCM/MeOH (20/1) to afford the sub-title compound (AAL-2) (280 mg, 793 μmol, 79%) as a white solid. m/z 354.2 (M+H)⁺ (ES+).

Step 2: 4′-Fluoro-2′-(4-methyl-1,2,4-triazol-3-yl)-[1,1′-biphenyl]-3-carboxylic acid (AAL-3). A solution of the product from step 1 above (AAL-2) (280 mg, 1 Eq, 793 μmol) and TFA (3 mL) in DCM (15 mL) was stirred for 1 h at rt. The resulting mixture was concentrated in vacuo. The crude product was used in the next step directly without further purification. m/z 298.1 (M+H)⁺ (ES+).

Step 3: N-(5-{5-Azaspiro[2.4]-heptan-5-ylmethyl}-2-hydroxy-3-(trifluoromethyl)-phenyl)-4′-fluoro-2′-(4-methyl-1,2,4-triazol-3-yl)-[1,1′-biphenyl]-3-carboxamide (AAL-4). To a stirred solution of the product from step 2 above (AAL-3) (100 mg, 1 Eq, 330 μmol) and intermediate (AAC-2) (96 mg, 1 Eq, 330 μmol) in DMF (10 mL) were added HATU (256 mg, 2 Eq, 670 μmol) and DIPEA (261 mg, 6 Eq, 2.01 mmol) at rt. The resulting mixture was stirred for 16 h at rt. The residue was purified by reverse flash chromatography with the following conditions: column, C18 silica gel; mobile phase, Water (0.1% NH₄HCO₃) and MeCN (30% MeCN up to 50% in 10 min); detector, UV 254 nm. This resulted in the sub-title compound (AAL-4) (25 mg, 44 μmol, 13%) as a green solid. m/z 566.2 (M+H)⁺ (ES+).

Step 4: 5-((5-Azaspiro[2.4]heptan-5-yl)methyl)-2-(4′-fluoro-2′-(4-methyl-4H-1,2,4-triazol-3-yl)-[1,1′-biphenyl]-3-yl)-7-(trifluoromethyl)benzo[d]oxazole (AAL-5). To a stirred solution of the product from step 3 above (AAL-4) (20 mg, 1 Eq, 30 μmol) and PPh₃ (37 mg, 4 Eq, 140 μmol) in THF (5 mL) was added DIAD (29 mg, 4 Eq, 140 μmol) at rt under. The resulting mixture was stirred for 3 h at 60° C. The resulting mixture was cooled to rt. The crude product was purified by Prep-HPLC with the following conditions: Column: SunFire Prep C18 OBD Column, 30*150 mm, 5 um; Mobile Phase A: Water (0.05% TFA), Mobile Phase B: MeCN; Flow rate: 60 mL/min; Gradient: 16 B to 28 B in 10 min; Detector, UV 254/210 nm; RT: 10.5. The product-containing fractions were combined and evaporated partially in vacuo and lyophilized overnight to afford the title compound (AAL-5) (1.5 mg, 2.7 μmol, 7%) as a white solid. m/z 548.3 (M+H)⁺ (ES+). ¹H NMR (400 MHz, Methanol-d4) δ 8.40 (s, 1H), 8.24 (d, J=7.8 Hz, 1H), 8.09-8.04 (m, 2H), 7.85-7.76 (m, 2H), 7.64-7.53 (m, 2H), 7.52-7.44 (m, 2H), 4.06 (s, 2H), 3.27 (s, 3H), 3.03 (s, 2H), 2.77 (s, 2H), 1.96 (t, J=7.1 Hz, 2H), 0.65 (d, J=4.4 Hz, 4H).

### Example 92: Synthesis of 6-(6-{5-Azaspiro[2.4]heptan-5-ylmethyl}-4-(trifluoromethyl)-3H-1,3-benzodiazol-2-yl)-N-ethyl-4-[2-methyl-4-(4-methyl-1,2,4-triazol-3-yl)pyrazol-3-yl]pyridin-2-amine (AAM-2)

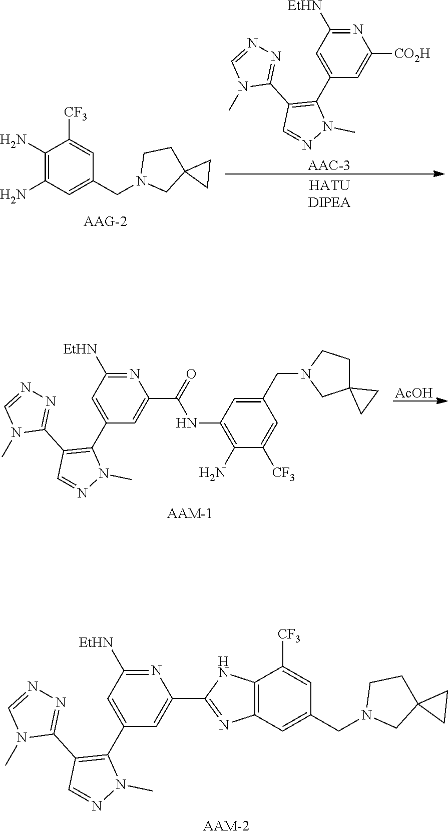

Step 1: N-(2-Amino-5-{5-azaspiro[2.4]heptan-5-ylmethyl}-3-(trifluoromethyl)phenyl)-6-(ethylamino)-4-[2-methyl-4-(4-methyl-1,2,4-triazol-3-yl)pyrazol-3-yl]pyridine-2-carboxamide (AAM-1). To a stirred solution of intermediate (AAC-3) (117 mg, 1 Eq, 357 μmol) and intermediate (AAG-2) (153 mg, 1.5 Eq, 535 μmol) in DMF (10 mL) were added HATU (177 mg, 1.3 Eq, 464 μmol) and DIPEA (139 mg, 3 Eq, 1.07 mmol) at rt. The resulting mixture was stirred for 1 h at rt. The resulting mixture was diluted with water (50 mL) and extracted with EtOAc (3×20 mL). The combined organic layers were washed with brine (3×20 mL), dried (Na₂SO₄) and concentrated in vacuo. The residue was purified by Prep-TLC with DCM/MeOH (6/1) to afford the sub-title compound (AAM-1) (123 mg, 207 μmol, 58%) as a white solid. m/z 595.3 (M+H)⁺ (ES+).

Step 2: 6-(6-{5-Azaspiro[2.4]heptan-5-ylmethyl}-4-(trifluoromethyl)-3H-1,3-benzodiazol-2-yl)-N-ethyl-4-[2-methyl-4-(4-methyl-1,2,4-triazol-3-yl)pyrazol-3-yl]pyridin-2-amine (AAM-2). A solution of the product from step 1 above (AAM-1) (123 mg, 1 Eq, 207 μmol) in AcOH (10 mL) was stirred for 2 h at 80° C. The mixture was allowed to cool down to rt and concentrated in vacuo. The crude product was purified by Prep-HPLC with the following conditions (Column: XBridge Prep OBD C18 Column, 30*150 mm, 5 μm; Mobile Phase A: Water (0.1% NH₄HCO₃), Mobile Phase B: MeCN; Flow rate: 60 mL/min; Gradient: 35% B to 50% B in 10 min; Wave Length: 254/220 nm). This resulted in the sub-title compound (AAM-2) (40.1 mg, 70 μmol, 33%) as a white solid. m/z 577.1 (M+H)⁺ (ES+). ¹H NMR (300 MHz, DMSO-d6) δ 12.84 (s, 1H), 8.46 (s, 1H), 7.94 (s, 1H), 7.83 (s, 1H), 7.48 (s, 1H), 7.35 (s, 1H), 7.00 (t, J=5.5 Hz, 1H), 6.59 (s, 1H), 3.92-3.74 (m, 5H), 3.62-3.47 (m, 5H), 2.71 (t, J=6.5 Hz, 2H), 2.47 (s, 2H), 1.77 (t, J=6.8 Hz, 2H), 1.22 (t, J=7.2 Hz, 3H), 0.51 (d, J=4.9 Hz, 4H).

### Example 93: Synthesis of {2-[6-(Ethylamino)-4-[2-(4-methyl-1,2,4-triazol-3-yl) phenyl]pyridin-2-yl]-7-(trifluoromethyl)-1H-1,3-benzodiazol-5-yl}methanol (AAN-3)

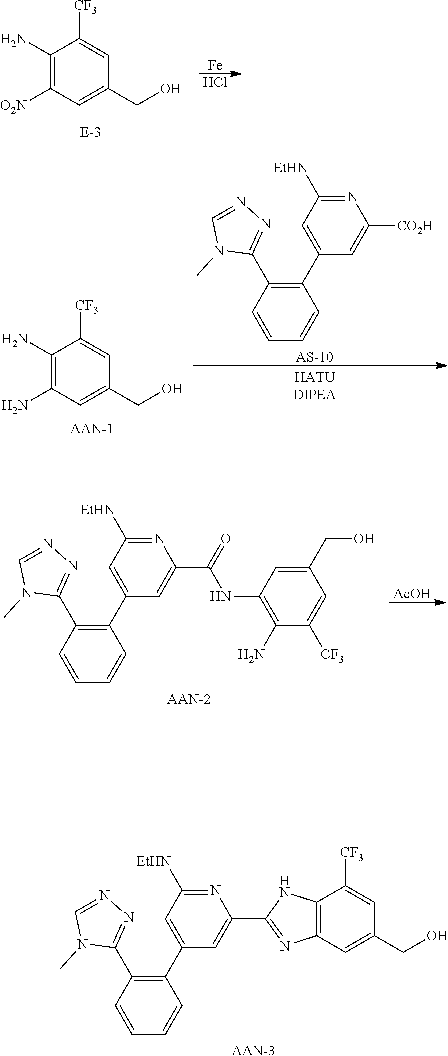

Step 1: [3,4-Diamino-5-(trifluoromethyl)phenyl]methanol (AAN-1). To a stirred solution of conc. HCl (3 mL) and water (3 mL) was added intermediate (E-3) (120 mg, 1 Eq, 508 μmol) at 0° C. under nitrogen atmosphere. To the above mixture was added Fe powder (170 mg, 6 Eq, 3.05 mmol) at 0° C. The resulting mixture was stirred for additional 30 min at rt. The crude product was purified by reverse flash column chromatography with the following conditions: Column, C18; mobile phase, Water (0.1% FA) and MeCN (0% MeCN up to 20% in 20 min); Detector, UV 254/220 nm. This resulted in the sub-title compound (AAN-1) (28 mg, 136 μmol, 27%) as a brown solid. m/z 207.1 (M+H)⁺ (ES+).

Step 2: N-[2-Amino-5-(hydroxymethyl)-3-(trifluoromethyl)phenyl]-6-(ethylamino)-4-[2-(4-methyl-1,2,4-triazol-3-yl)phenyl]pyridine-2-carboxamide (AAN-2). To a stirred solution of intermediate (AS-10) (35 mg, 1 Eq, 108 μmol) in DMF (5 mL) was added DIPEA (42 mg, 3 Eq, 324 μmol) dropwise at rt under nitrogen atmosphere. The resulting mixture was stirred for 10 min at rt under nitrogen atmosphere. To the above mixture was added the product from step 1 above (AAN-1) (27 mg, 1.2 Eq, 130 μmol) at rt. The resulting mixture was stirred for additional 1 h at rt. The resulting mixture was diluted with water and extracted with EtOAc (3×20 mL). The combined organic layers were washed with brine (2×20 mL), dried (Na₂SO₄). After filtration, the filtrate was concentrated in vacuo. The residue was purified by Prep-TLC with DCM/MeOH (10/1) to afford the sub-title compound (AAN-2) (41 mg, 80 μmol, 65%) as a yellow solid. m/z 512.2 (M+H)⁺ (ES+).

Step 3: {2-[6-(Ethylamino)-4-[2-(4-methyl-1,2,4-triazol-3-yl)phenyl]pyridin-2-yl]-7-(trifluoromethyl)-1H-1,3-benzodiazol-5-yl}methanol (AAN-3). The product from step 3 above (AAN-2) (35 mg, 1 Eq, 68 μmol) was added in AcOH (3 mL) at rt under nitrogen atmosphere. The resulting mixture was stirred for 1 h at 80° C. under nitrogen atmosphere. The mixture was cooled to rt and concentrated in vacuo. The crude product was purified by Prep-HPLC with the following conditions (Column: XBridge Prep OBD C18 Column, 30*150 mm, 5 μm; Mobile Phase A: Water (0.1% NH₄HCO₃), Mobile Phase B: MeCN; Flow rate: 60 mL/min; Gradient: 34% B to 52% B in 8 min; Wavelength: 254/220 nm; RT: 7.6) to afford the title compound (AAN-3) (12.0 mg, 24 μmol, 35%) as a light yellow solid. m/z 494.0 (M+H)⁺ (ES+). ¹H NMR (400 MHz, DMSO-d6) δ 12.75 (s, 1H), 8.45 (s, 1H), 7.81 (s, 1H), 7.71-7.76 (m, 1H), 7.71-7.63 (m, 2H), 7.62-7.58 (m, 1H), 7.50 (s, 1H), 7.33 (d, J=1.4 Hz, 1H), 6.74 (t, J=5.4 Hz, 1H), 6.20 (d, J=1.4 Hz, 1H), 5.40 (t, J=5.8 Hz, 1H), 4.68 (d, J=5.8 Hz, 2H), 3.50-3.35 (m, 2H), 3.30 (s, 3H), 1.17 (t, J=7.2 Hz, 3H).

### Example 94: Synthesis of 2-(6-(Ethylamino)-4-(2-(4-methyl-4H-1,2,4-triazol-3-yl) phenyl)pyridin-2-yl)-8-(trifluoromethyl)-[1,2,4]triazolo[4,3-a]pyridin-3(2H)-one (AAO-4)

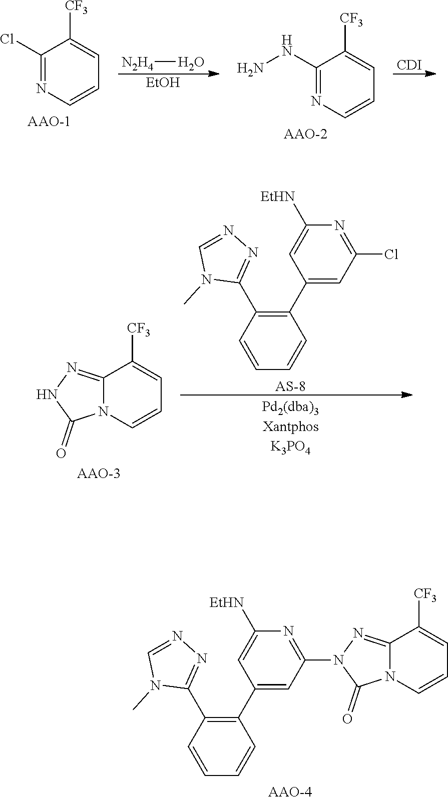

Step 1: 2-Hydrazineyl-3-(trifluoromethyl) pyridine (AAO-2). To a stirred solution of 2-chloro-3-(trifluoromethyl)pyridine (AAO-1) (362 mg, 1 Eq, 1.99 mmol) in EtOH (2 mL) was added hydrazine hydrate (1.50 g, 98% Wt, 15 Eq, 30.0 mmol) at rt under nitrogen atmosphere. The resulting mixture was stirred for 3 h at 80° C. under nitrogen atmosphere. The mixture was allowed to cool down to rt and concentrated in vacuo. The residue was purified by Prep-TLC with EtOAc/petroleum ether (1/1) to afford the sub-title compound (AAO-2) (240 mg, 1.36 mmol, 68%) as a grey solid. m/z 178.1 (M+H)⁺ (ES+).

Step 2: 8-(Trifluoromethyl)-[1,2,4]triazolo[4,3-a]pyridin-3(2H)-one (AAO-3). To a stirred solution of the product from step 1 above (AAO-2) (130 mg, 1 Eq, 734 μmol) in DCM (6 mL) was added CDI (179 mg, 1.5 Eq, 1.10 mmol) at rt under nitrogen atmosphere. The resulting mixture was stirred for 3 h at rt under nitrogen atmosphere. The mixture was allowed to cool down to rt and concentrated in vacuo. The crude product was purified by reverse flash column chromatography with the following conditions: Column, C18; mobile phase, Water (0.1% FA) and MeCN (10% MeCN up to 80% in 20 min); Detector, UV 254/220 nm to afford the sub-title compound (AAO-3) (80 mg, 394 μmol, 54%) as a white solid. m/z 204.0 (M+H)⁺ (ES+).

Step 3: 2-(6-(Ethylamino)-4-(2-(4-methyl-4H-1,2,4-triazol-3-yl)phenyl)pyridin-2-yl)-8-(trifluoromethyl)-[1,2,4]triazolo[4,3-a]pyridin-3(2H)-one (AAO-4). To a solution of intermediate (AS-8) (70 mg, 1 Eq, 223 μmol), potassium phosphate (118 mg, 2.5 Eq, 557 μmol) and the product from step 2 above (AAO-3) (50 mg, 1.1 Eq, 245 μmol) in 1,4-dioxane (6 mL) were added Pd₂(dba)₃-CHCl₃ (46 mg, 0.2 Eq, 45 μmol) and XantPhos (52 mg, 0.4 Eq, 89 μmol), under a nitrogen atmosphere. The resulting mixture was stirred for overnight at 100° C. under nitrogen atmosphere. The mixture was allowed to cool down to rt and concentrated. The residue was purified by Prep-TLC with DCM/MeOH (10/1) and Prep-HPLC with the following conditions: (Column: XBridge Prep OBD C18 Column, 30*150 mm, 5 μm; Mobile Phase A: Water (0.11% NH₄HCO₃), Mobile Phase B: MeCN; Flow rate: 60 mL/min; Gradient: 33% B to 44% B in 8 min; Wave Length: 254/220 nm; RT: 7.7). The product-containing fractions were combined and evaporated partially in vacuo and lyophilized overnight to afford the sub-title compound (AAO-4) (24.6 mg, 51 μmol, 23%) as a yellow solid. m/z 481.1 (M+H)⁺ (ES+). ¹H NMR (300 MHz, Methanol-d4) δ 8.46 (s, 1H), 8.09 (d, J=7.1 Hz, 1H), 7.84-7.72 (m, 3H), 7.72-7.59 (m, 2H), 7.05 (s, 1H), 6.77 (t, J=7.0 Hz, 1H), 6.18 (s, 1H), 3.38 (s, 3H), 3.27-3.16 (m, 2H), 1.21 (t, J=7.2 Hz, 3H).

### Example 95: Synthesis of 2-{6-Cyclopropyl-4-[2-(4-methyl-1,2,4-triazol-3-yl)phenyl]pyridin-2-yl}-5-{[(3S)-3-methylpiperidin-1-yl]methyl}-7-(trifluoromethyl)-1,3-benzoxazole (AAP-7)

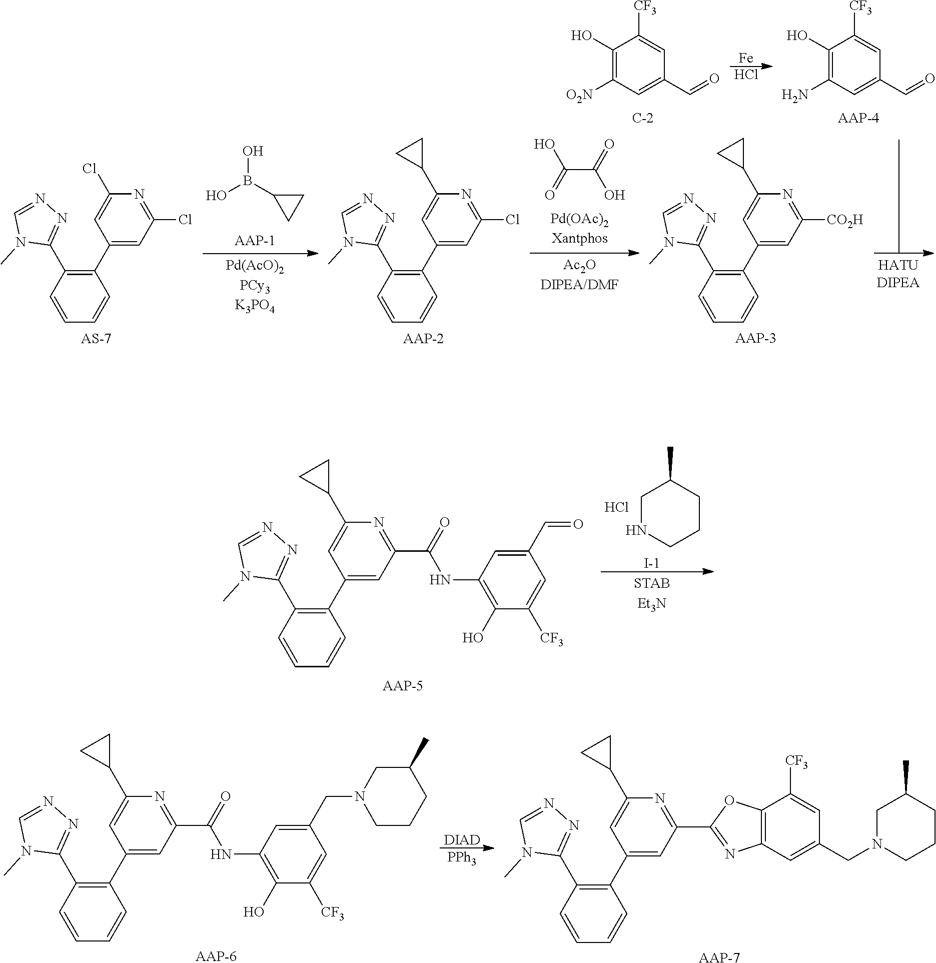

Step 1: 2-Chloro-6-cyclopropyl-4-[2-(4-methyl-1,2,4-triazol-3-yl)-phenyl]-pyridine (AAP-2). To a stirred solution of intermediate (AS-7) (450 mg, 1 Eq, 1.47 mmol) and cyclopropylboronic acid (AAP-1) (177 mg, 1.4 Eq, 2.06 mmol) in toluene (15 mL) were added potassium phosphate (939 mg, 3 Eq, 4.45 mmol), PCy₃ (62 mg, 0.1 Eq, 220 μmol) and Pd(AcO)₂ (50 mg, 0.1 Eq, 220 μmol) at rt under nitrogen atmosphere. The resulting mixture was stirred for 16 hours at 110° C. under nitrogen atmosphere. The mixture was cooled to rt and concentrated in vacuo. The crude product was purified by reverse flash column chromatography with the following conditions: C18 silica gel; mobile phase, Water (0.1% NH₄HCO₃) and MeCN (20% MeCN up to 40% in 10 min); Detector, UV 254 nm. This resulted in the sub-title compound (AAP-2) (120 mg, 387 μmol, 26%) as a white solid. m/z 311.1/313.1 (M+H)⁺ (ES+).

Step 2: 6-Cyclopropyl-4-[2-(4-methyl-1,2,4-triazol-3-yl)-phenyl]-pyridine-2-carboxylic acid (AAP-3). To a stirred solution of the product from step 1 above (AAP-2) (90 mg, 1 Eq, 290 μmol) and oxalic acid (39 mg, 1.5 Eq, 430 μmol) in DMF (10 mL) were added Ac₂O (44 mg, 1.5 Eq, 430 μmol), DIPEA (56 mg, 1.5 Eq, 430 μmol), Pd(AcO)₂ (6.5 mg, 0.1 Eq, 20 μmol) and XantPhos (34 mg, 0.2 Eq, 50 μmol) at rt under nitrogen atmosphere. The resulting mixture was stirred for 4 hours at 100° C. under nitrogen atmosphere. The mixture was cooled to rt and concentrated in vacuo. The crude product was purified by reverse flash column chromatography with the following conditions: C18 silica gel; mobile phase, Water (0.1% FA) and MeCN (10% MeCN up to 30% in 10 min); detector, UV 254 nm. This resulted in the sub-title compound (AAP-3) (67 mg, 209 μmol, 72%) as a white solid. m/z 321.1 (M+H)⁺ (ES+).

Step 3: 3-Amino-4-hydroxy-5-(trifluoromethyl)benzaldehyde (AAP-4). To a stirred solution of conc. HCl (aq.) (20 mL, 6 molar) were added intermediate (C-2) (450 mg, 1 Eq, 1.91 mmol) and Fe powder (641 mg, 11.5 mmol, 6 Eq) at 0° C. The resulting mixture was stirred for 2 h at 0° C. The crude product was purified by reverse flash column chromatography with the following conditions: Column, C18; mobile phase, Water (0.1% FA) and MeCN (0% MeCN up to 10% in 20 min); Detector, UV 254/220 nm. This resulted in the sub-title compound (AAP-4) (200 mg, 976 μmol, 51%) as a brown oil. m/z 206.0 (M+H)⁺ (ES+).

Step 4: 6-Cyclopropyl-N-(5-formyl-2-hydroxy-3-(trifluoromethyl)phenyl)-4-(2-(4-methyl-4H-1,2,4-triazol-3-yl)phenyl)picolinamide (AAP-5). Into a 100-mL round-bottom flask and maintained with an inert atmosphere of nitrogen, was placed the product from step 2 above (AAP-3) (170 mg, 1 Eq, 531 μmol) and the product from step 3 above (AAP-4) (109 mg, 1 Eq, 531 μmol) in DMF (10 mL) at rt, then HATU (222 mg, 1.1 Eq, 584 μmol) and DIPEA (206 mg, 3 Eq, 1.59 mmol) were added at 0° C. under nitrogen atmosphere. The resulting mixture was stirred for 3 h at rt. The resulting mixture was diluted with water and extracted with EtOAc (3×20 mL). The combined organic layers were washed with brine (2×20 mL), dried (Na₂SO₄). After filtration, the filtrate was concentrated in vacuo. The residue was purified by Prep-TLC with DCM/MeOH (10/1) to afford the sub-title compound (AAP-5) (150 mg, 296 μmol, 56%) as a white solid. m/z 508.2 (M+H)⁺ (ES+).

Step 5: (S)-6-Cyclopropyl-N-(2-hydroxy-5-((3-methylpiperidin-1-yl)methyl)-3-(trifluorome thyl)phenyl)-4-(2-(4-methyl-4H-1,2,4-triazol-3-yl)phenyl)picolinamide (AAP-6). Into a 250-mL round-bottom flask and maintained with an inert atmosphere of nitrogen, was placed the product from step 4 above (AAP-5) (70 mg, 1 Eq, 138 μmol) and (S)-3-methylpiperidine, HCl (I-1) (75 mg, 4 Eq, 552 μmol) and Et₃N (42 mg, 3 Eq, 414 μmol) in DCM (10 mL). The resulting mixture was stirred for 30 min at rt under nitrogen atmosphere, and then NaBH(OAc)₃ (59 mg, 2 Eq, 276 μmol) was added at rt. The resulting mixture was stirred for 2 h at rt under nitrogen atmosphere. The resulting mixture was diluted with water and extracted with DCM (3×20 mL). The combined organic layers were washed with brine (2×20 mL), dried (Na₂SO₄). After filtration, the filtrate was concentrated in vacuo. The crude product was purified by reverse flash column chromatography with the following conditions: Column, C18; mobile phase, Water (0.1% NH₄HCO₃) and MeCN (0% MeCN up to 50% in 10 min); Detector, UV 254/220 nm to afford the sub-title compound (AAP-6) (50 mg, 85 μmol, 61%) as a white solid. m/z 591.3 (M+H)⁺ (ES+).

Step 6: (S)-2-(6-Cyclopropyl-4-(2-(4-methyl-4H-1,2,4-triazol-3-yl)phenyl)pyridin-2-yl)-5-((3-methylpiperidin-1-yl)methyl)-7-(trifluoromethyl)benzo[d]oxazole (AAP-7). Into a 20-mL vial purged and maintained with an inert atmosphere of nitrogen, was placed the product from step 5 above (AAP-6) (40 mg, 1 Eq, 68 μmol) in THF (10 mL), then PPh₃ (36 mg, 2 Eq, 136 μmol) and DIAD (27 mg, 2 Eq, 136 μmol) were added at rt under nitrogen atmosphere. The resulting mixture was stirred for 5 h at 60° C. under nitrogen atmosphere. The mixture was allowed to cool down to rt. The resulting mixture was diluted with water and extracted with DCM (3×20 mL). The combined organic layers were washed with brine (2×20 mL), dried (Na₂SO₄). After filtration, the filtrate was concentrated in vacuo. The crude product was purified by Prep-HPLC with the following conditions (Column: XBridge Prep OBD C18 Column, 30×150 mm, 5 μm; Mobile Phase A: Water (0.1% NH₄HCO₃), Mobile Phase B: MeCN; Flow rate: 60 mL/min; Gradient: 55% B to 90% B in 9 min; Wave Length: 254/220 nm; RT: 8.32). This resulted in the title compound (AAP-7) (5.2 mg, 9.1 μmol, 13%) as a white solid. m/z 573.5 (M+H)⁺ (ES+). ¹H NMR (400 MHz, DMSO-d6) δ 8.47 (s, 1H), 8.07 (s, 1H), 7.83-7.64 (m, 6H), 7.18 (d, J=1.6 Hz, 1H), 3.65 (s, 2H), 3.27 (s, 3H), 2.74 (t, J=8.1 Hz, 2H), 2.24-2.13 (m, 1H), 1.94 (t, J=10.8 Hz, 1H), 1.68-1.55 (m, 4H), 1.50 (t, J=12.0 Hz, 1H), 1.10-1.01 (m, 2H), 0.97-0.90 (m, 2H), 0.89-0.80 (m, 4H).

### Example 96: Synthesis of 2-{6-Cyclopropyl-4-[2-(4-methyl-1,2,4-triazol-3-yl)phenyl]pyridin-2-yl}-5-{[(3R)-3-methylpiperidin-1-yl]methyl}-7-(trifluorometh yl)-1,3-benzoxazole (AAQ-2)

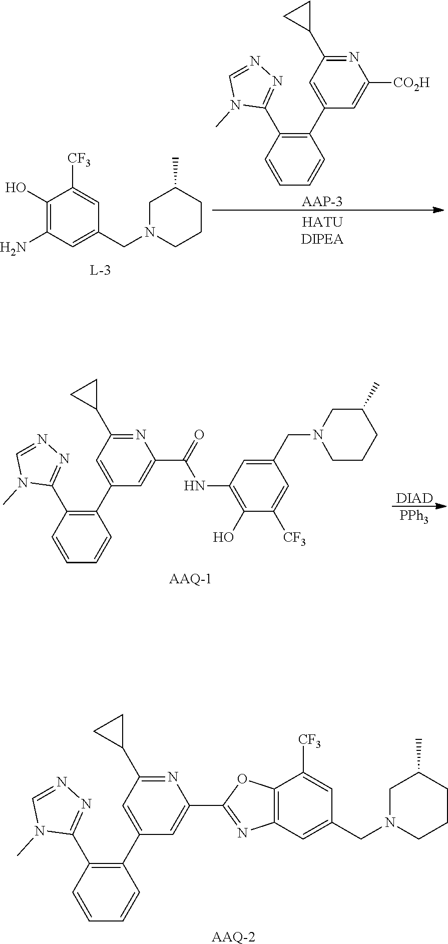

Step 1: 6-Cyclopropyl-N-(2-hydroxy-5-{[(3R)-3-methylpiperidin-1-yl]methyl}-3-(trifluoromethyl)phenyl)-4-[2-(4-methyl-1,2,4-triazol-3-yl)phenyl]pyridine-2-carboxamide (AAQ-1). To a stirred solution of intermediate (AAP-3) (40 mg, 1 Eq, 125 μmol) and intermediate (1-3) (54 mg, 1.5 Eq, 188 μmol) in DMF (5 mL) was added HATU (142 mg, 3 Eq, 375 μmol) and DIPEA (48 mg, 3 Eq, 375 μmol) at rt. The resulting mixture was stirred for 2 h at rt. The crude product was purified by reverse flash column chromatography with the following conditions: Column, C18; mobile phase, Water (0.1% NH₄HCO₃) and MeCN (0% MeCN up to 50% in 10 min); Detector, UV 254/220 nm. This resulted in the sub-title compound (AAQ-1) (25 mg, 42 μmol, 31%) as a white solid. m/z 591.3 (M+H)⁺ (ES+).

Step 2: 2-{6-Cyclopropyl-4-[2-(4-methyl-1,2,4-triazol-3-yl)phenyl]pyridin-2-yl}-5-{[(3R)-3-methylpiperidin-1-yl]methyl}-7-(trifluoromethyl)-1,3-benzoxazole (AAQ-2). To a stirred solution of the product from step 1 above (AAQ-1) (25 mg, 1 Eq, 42 μmol) and PPh₃ (33 mg, 3 Eq, 126 μmol) in THF (4 mL) was added DIAD (26 mg, 3 Eq, 126 μmol) at rt under nitrogen atmosphere. The resulting mixture was stirred for 2 h at 60° C. under nitrogen atmosphere. The mixture was cooled to rt and concentrated in vacuo. The crude product was purified by Prep-HPLC with the following conditions (Column: XBridge Prep OBD C18 Column, 30*150 mm, 5 μm; Mobile Phase A: Water (0.1% NH₄HCO₃), Mobile Phase B: MeCN; Flow rate: 60 mL/min; Gradient: 55% B to 90% B in 9 min; Wave Length: 254/220 nm; RT: 8.32) to afford the title compound (AAQ-2) (6.7 mg, 12 μmol, 27%) as a white solid. m/z 573.3 (M+H)⁺ (ES+). ¹H NMR (400 MHz, Methanol-d4) δ 8.47 (s, 1H), 8.05 (s, 1H), 7.89 (d, J=1.6 Hz, 1H), 7.88-7.78 (m, 3H), 7.75-7.71 (m, 2H), 7.21 (d, J=1.6 Hz, 1H), 3.75 (s, 2H), 3.36 (s, 3H), 2.89 (s, 2H), 2.26-2.15 (m, 1H), 2.05 (s, 1H), 1.77-1.50 (m, 5H), 1.13-1.06 (m, 4H), 0.96-0.90 (m, 4H).

### Example 97: Synthesis of 5-{5-Azaspiro[2.4]-heptan-5-ylmethyl}-2-{6-cyclopropyl-4-[2-(4-methyl-1,2,4-triazol-3-yl)-phenyl]-pyridin-2-yl}-7-(trifluoromethyl)-1,3-benzoxazole (AAR-2)

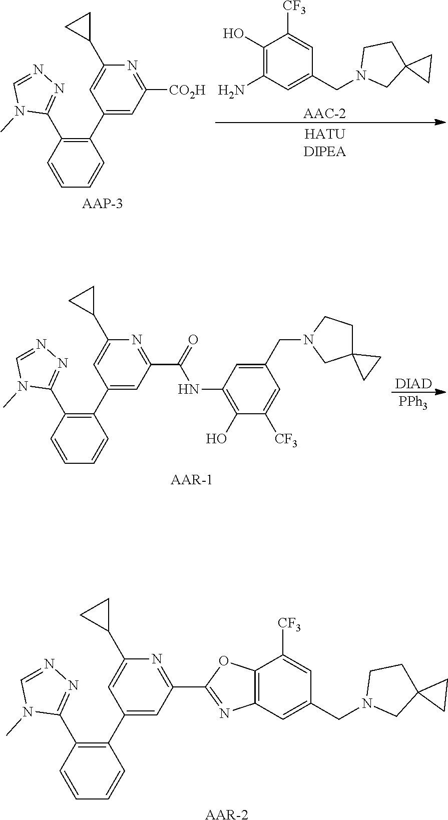

Step 1: N-(5-{5-Azaspiro[2.4]-heptan-5-ylmethyl}-2-hydroxy-3-(trifluoromethyl)-phenyl)-6-cyclopropyl-4-[2-(4-methyl-1,2,4-triazol-3-yl)-phenyl]-pyridine-2-carboxamide (AAR-1). To a stirred solution of intermediate (AAP-3) (40 mg, 1 Eq, 120 μmol) and intermediate (AAC-2) (43 mg, 1.2 Eq, 150 μmol) in DMF (5 ml) were added HATU (95 mg, 2 Eq, 250 μmol) and DIPEA (97 mg, 6 Eq, 750 μmol) at rt. The resulting mixture was stirred for 3 hours at rt. The crude product was purified by reverse flash column chromatography with the following conditions: C18 silica gel; mobile phase, Water (0.1% NH₄HCO₃) and MeCN (40% MeCN up to 60% in 10 min); detector, UV 254 nm. This resulted in the sub-title compound (AAR-1) (25 mg, 43 μmol, 34%) as an orange solid. m/z 589.2 (M+H)⁺ (ES+).

Step 2: 5-{5-Azaspiro[2.4]-heptan-5-ylmethyl}-2-{6-cyclopropyl-4-[2-(4-methyl-1,2,4-triazol-3-yl)-phenyl]-pyridin-2-yl}-7-(trifluoromethyl)-1,3-benzoxazole (AAR-2). To a stirred solution of the product from step 1 (AAR-1) (25 mg, 1 Eq, 40 μmol) and DIAD (34 mg, 4 Eq, 160 μmol) in THF (5 mL) was added PPh₃ (22 mg, 2 Eq, 80 mmol) at rt under nitrogen atmosphere. The resulting mixture was stirred for 16 hours at 60° C. under nitrogen atmosphere. The mixture was cooled to rt and concentrated in vacuo. The crude product was purified by Prep-HPLC with the following conditions: Column: SunFire Prep C18 OBD Column, 30*150 mm, 5 um; Mobile Phase A: Water (0.1% FA), Mobile Phase B: MeCN; Flow rate: 60 mL/min; Gradient: 11 B to 35 B in 7 min; Detector, UV 254/210 nm; RT: 7. The product-containing fractions were combined and evaporated partially in vacuo and lyophilized overnight to afford the title compound (AAR-2) (11.4 mg, 20 μmol, 46%) as a white solid. m/z 571.4 (M+H)⁺ (ES+). ¹H NMR (400 MHz, Methanol-d4) δ 8.47 (s, 1H), 8.08 (s, 1H), 7.89 (d, J=1.6 Hz, 1H), 7.88-7.79 (m, 3H), 7.79-7.68 (m, 2H), 7.21 (d, J=1.6 Hz, 1H), 3.92 (s, 2H), 3.36 (s, 3H), 2.89 (t, J=7.0 Hz, 2H), 2.63 (s, 2H), 2.25-2.16 (m, 1H), 1.91 (t, J=7.0 Hz, 2H), 1.09 (d, J=6.6 Hz, 4H), 0.66-0.56 (m, 4H).

### Example 98: Synthesis of 2-[6-(Ethylamino)-4-[2-methyl-4-(4-methyl-1,2,4-triazol-3-yl)pyrazol-3-yl]pyridin-2-yl]-6-{[(3R)-3-methylpiperidin-1-yl]methyl}-4-(trifluoromethyl)-3H-isoindol-1-one (AAS-2)

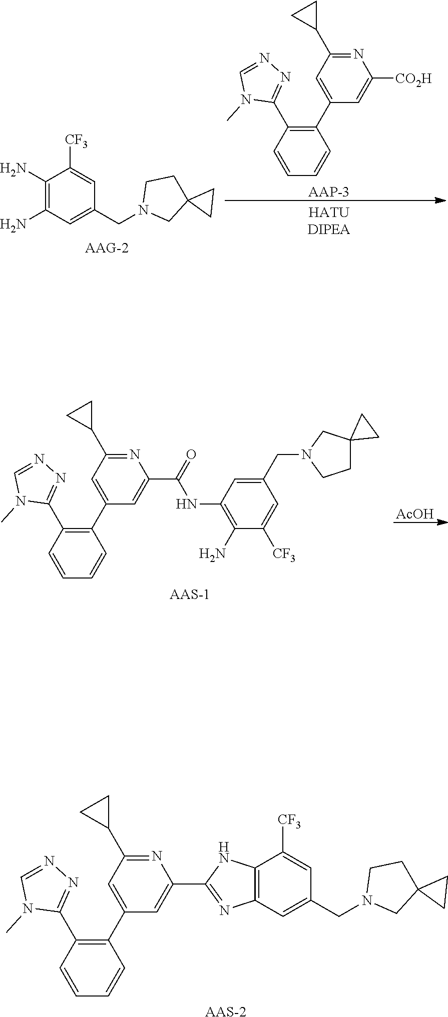

Step 1: N-(5-((5-Azaspiro[2.4]heptan-5-yl)methyl)-2-amino-3-(trifluoromethyl)phenyl)-6-cyclopropyl-4-(2-(4-methyl-4H-1,2,4-triazol-3-yl)phenyl)picolinamide (AAS-1). Into a 250-mL round-bottom flask purged and maintained with an inert atmosphere of nitrogen, was placed intermediate (AAG-2) (73 mg, 1 Eq, 228 μmol) and intermediate (AAP-3) (65. mg, 1 Eq, 228 μmol) in DMF (8 mL), then HATU (87 mg, 1 Eq, 228 μmol) and DIPEA (88 mg, 3 Eq, 684 μmol) were added at 0° C. under nitrogen atmosphere. The resulting mixture was stirred for 2 h at rt under nitrogen atmosphere. The resulting mixture was diluted with water and extracted with DCM (3×20 mL). The combined organic layers were washed with brine (2×20 mL), dried (Na₂SO₄). After filtration, the filtrate was concentrated in vacuo. The crude product was purified by reverse flash column chromatography with the following conditions: Column, C18; mobile phase, Water (0.1% NH₄HCO₃) and MeCN (0% MeCN up to 50% in 10 min); Detector, UV 254/220 nm. This resulted in the sub-title compound (AAS-1) (80 mg, 136 μmol, 60%) as a white solid. m/z 588.3 (M+H)⁺ (ES+).

Step 2: 5-((5-Azaspiro[2.4]heptan-5-yl)methyl)-2-(6-cyclopropyl-4-(2-(4-methyl-4H-1,2,4-triazol-3-yl)phenyl)pyridin-2-yl)-7-(trifluoromethyl)-1H-benzo[d]imidazole (AAS-2). Into a 20-mL vial purged and maintained with an inert atmosphere of nitrogen, was placed the product from step 1 above (AAS-1) (80 mg, 1 Eq, 136 μmol) in AcOH (5 mL). The resulting mixture was stirred for 2 h at 80° C. under nitrogen atmosphere. The mixture was cooled to rt. The resulting mixture was diluted with water and extracted with DCM (3×10 mL). The combined organic layers were washed with brine (2×10 mL), dried (Na₂SO₄). After filtration, the filtrate was concentrated in vacuo. The crude product was purified by Prep-HPLC with the following conditions (Column: XBridge Prep C18 OBD Column, 30×100 mm, 5 μm; Mobile Phase A: Water (0.1% NH₄HCO₃), Mobile Phase B: MeCN; Flow rate: 60 mL/min; Gradient: 42% B to 62% B in 10 min; Wave Length: 254/210 nm; RT: 9.77). This resulted in the title compound (AAS-2) (28 mg, 49 μmol, 35%) as a white solid. m/z 570.6 (M+H)⁺ (ES+). ¹H NMR (400 MHz, DMSO-d6) δ 13.06 (s, 1H), 8.46 (s, 1H), 7.87-7.63 (m, 6H), 7.51 (s, 1H), 6.99 (d, J=1.6 Hz, 1H), 3.78 (s, 2H), 3.31 (s, 3H), 2.71 (s, 2H), 2.47 (s, 2H), 2.16-2.07 (m, 1H), 1.78 (t, J=6.8 Hz, 2H), 1.10-0.98 (m, 4H), 0.51 (d, J=6.8 Hz, 4H).

### Example 99: Synthesis of (1r,3r)-3-((4-Methyl-4H-1,2,4-triazol-3-yl)methyl)-3-(3-(7-(trifluoromethyl)benzo[d]oxazol-2-yl)phenyl)cyclobutane-1-carbonitrile (AAT-14)

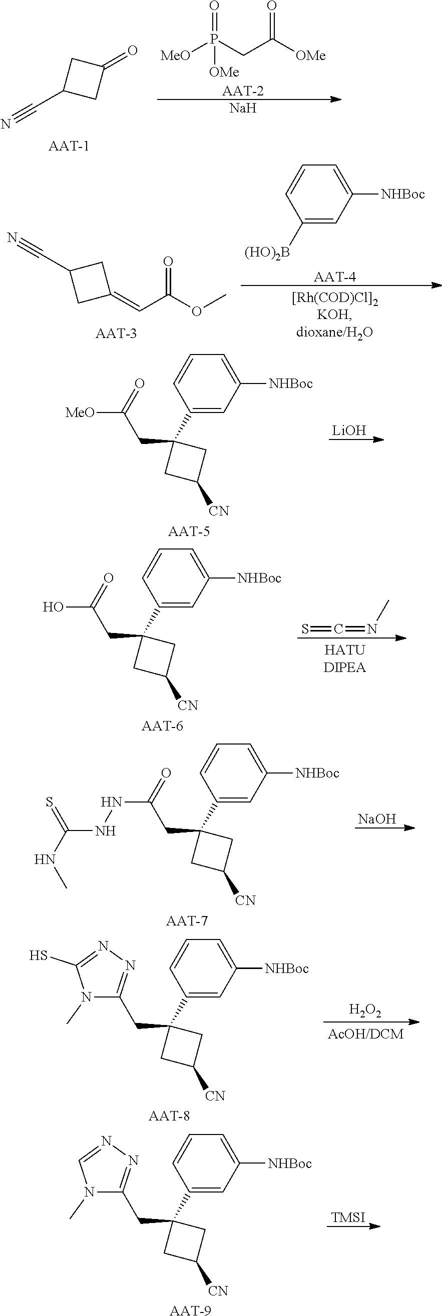

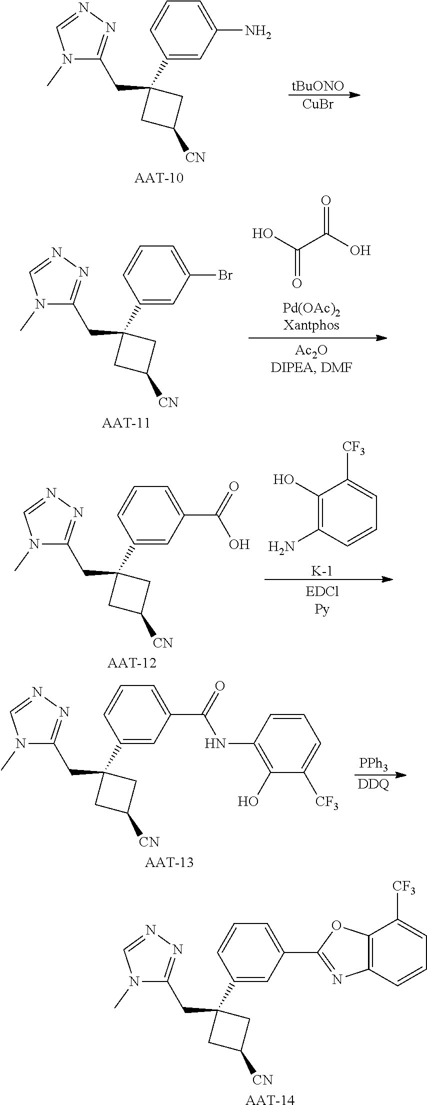

Step 1: Methyl 2-(3-cyanocyclobutylidene)acetate (AAT-3). To a stirred mixture of NaH (1.92 g, 1.2 Eq, 48.0 mmol) in THF (240 mL) was added a solution of methyl-2-(dimethoxyphosphoryl) acetate (AAT-2) (8.00 g, 1.1 Eq, 44.0 mmol) in THF (24 mL) dropwise at 0° C. under nitrogen atmosphere. The resulting mixture was stirred for 1 h at 0° C. under nitrogen atmosphere. To the above mixture was added a solution of 3-oxocyclobutane-1-carbonitrile (AAT-1) (3.80 g, 1 Eq, 40.0 mmol) in THF (24 mL) at 0° C. The resulting mixture was stirred overnight at rt. The reaction was poured into sat. NH₄Cl (aq., 400 mL) at 0° C. The resulting mixture was diluted with water and extracted with EtOAc (3×300 mL). The combined organic layers were washed with brine (2×300 mL), dried (Na₂SO₄). After filtration, the filtrate was concentrated in vacuo. The crude product was purified by reverse flash column chromatography with the following conditions: Column, C18; mobile phase, Water (0.1% NH₄HCO₃) and MeCN (10% MeCN up to 80% in 10 min); Detector, UV 254/220 nm to afford the sub-title compound (AAT-3) (3.2 g, 21.2 mmol, 53%) as a brown oil. m/z 152.1 (M+H)⁺ (ES+).

Step 2: Methyl 2-((1r,3r)-1-(3-((tert-butoxycarbonyl)amino)phenyl)-3-cyanocyclobutyl) acetate (AAT-5). To a stirred solution of the product from step 1 above (AAT-3) (2.8 g, 1 Eq, 18.5 mmol) in 1,4-dioxane (29 mL) was added a solution of KOH (1.35 g, 1.3 Eq, 24.1 mmol) in water (16 mL) at 0° C. under nitrogen atmosphere. To the above mixture was added 3-[(tert-butoxycarbonyl)amino]phenylboronic acid (AAT-4) (6.59 g, 1.5 Eq, 27.8 mmol) and [Rh(COD)Cl]₂ (457 mg, 0.05 Eq, 926 μmol) at 0° C. The resulting mixture was stirred for additional overnight at rt under nitrogen atmosphere. The reaction was poured into sat. NH₄Cl (aq., 200 mL) at 0° C. The resulting mixture was diluted with water and extracted with EtOAc (3×100 mL). The combined organic layers were washed with brine (2×100 mL), dried (Na₂SO₄). After filtration, the filtrate was concentrated in vacuo. The crude product was purified by reverse flash column chromatography with the following conditions: Column, C18; mobile phase, Water (0.1% NH₄HCO₃) and MeCN (10% MeCN up to 80% in 20 min); Detector, UV 254/220 nm and silica gel column chromatography with petroleum ether/EtOAc (4/1) to afford the sub-title compound (AAT-5) (900 mg, 2.61 mmol, 14%) as a white solid. m/z 345.2 (M+H)⁺ (ES+).

Step 3: 2-((1r,3r)-1-(3-((tert-Butoxycarbonyl)amino)phenyl)-3-cyanocyclobutyl)acetic acid (AAT-6). To a stirred solution of the product from step 2 above (AAT-5) (900 mg, 1 Eq, 2.61 mmol) in MeOH (18 mL) and THF (18 mL) was added a solution of LiOH—H₂O (548 mg, 5 Eq, 13.1 mmol) in water (9 mL) at 0° C. The resulting mixture was stirred for overnight at rt. The resulting mixture was diluted with water (120 mL). The mixture was acidified to pH 4 with HCl (aq., 1M). The resulting mixture was diluted with water and extracted with EtOAc (3×100 mL). The combined organic layers were washed with brine (2×100 mL), dried (Na₂SO₄). After filtration, the filtrate was concentrated in vacuo to afford the sub-title compound (AAT-6) (800 mg, 2.42 mmol, 93%) as a brown solid. m/z 331.2 (M+H)⁺ (ES+).

Step 4: tert-Butyl (3-((1r,3r)-3-cyano-1-(2-(2-(methylcarbamothioyl)hydrazineyl)-2-oxoethyl)cyclobutyl)phenyl)carbamate (AAT-7). To a stirred solution of the product from step 3 above (AAT-6) (800 mg, 1 Eq, 2.42 mmol) and 1-amino-3-methylthiourea (382 mg, 1.5 Eq, 3.63 mmol) in DMF (10 mL) were added DIPEA (939 mg, 3 Eq, 7.26 mmol) dropwise and HATU (1.10 g, 1.2 Eq, 2.91 mmol) at 0° C. The resulting mixture was stirred for 3 h at rt. The crude product was used in the next step directly without further purification. m/z 418.2 (M+H)⁺ (ES+).

Step 5: tert-Butyl (3-((1r,3r)-3-cyano-1-((5-mercapto-4-methyl-4H-1,2,4-triazol-3-yl) methyl)cyclobutyl)phenyl)carbamate (AAT-8). To a stirred solution of the product from step 4 above (AAT-7) (1.00 g, 1 Eq, 2.40 mmol) in DMF (10 mL) was added a solution of NaOH (960 mg, 10 Eq, 24.0 mmol) in water (24 mL) at 0° C. The resulting mixture was stirred for overnight at 50° C. The mixture was cooled down to rt and diluted with water (200 mL). The resulting mixture was diluted with water and extracted with EtOAc (3×100 mL). The combined organic layers were washed with brine (2×100 mL), dried (Na₂SO₄). After filtration, the filtrate was concentrated in vacuo. The residue was purified by reverse flash chromatography with the following conditions: (column, C18 silica gel; mobile phase, MeCN in water (0.1% NH₄HCO₃), 10% to 80% gradient in 20 min; detector, UV 254/220 nm) to afford the sub-title compound (AAT-8) (600 mg, 1.50 mmol, 63%) as a yellow solid. m/z 400.2 (M+H)⁺ (ES+).

Step 6: tert-Butyl (3-((1r,3r)-3-cyano-1-((4-methyl-4H-1,2,4-triazol-3-yl)methyl)cyclobutyl) phenyl)carbamate (AAT-9). To a stirred solution of the product from step 5 above (AAT-8) (600 mg, 1 Eq, 1.50 mmol) in DCM (12 mL) was added hydrogen peroxide (aq.) (255 mg, 30% Wt, 5 Eq, 7.51 mmol) and AcOH (180 mg, 2 Eq, 3.00 mmol) at 0° C. The resulting mixture was stirred for 2 h at rt. The mixture was concentrated in vacuo. The crude product was purified by reverse flash column chromatography with the following conditions: Column, C18; mobile phase, Water (0.1% NH₄HCO₃) and MeCN (10% MeCN up to 80% in 20 min); Detector, UV 254/220 nm to afford the sub-title compound (AAT-9) (450 mg, 1.23 mmol, 82%) as a yellow solid. m/z 368.2 (M+H)⁺ (ES+).

Step 7: (1r,3r)-3-(3-Aminophenyl)-3-((4-methyl-4H-1,2,4-triazol-3-yl)methyl)cyclobutane-1-carbonitrile (AAT-10). To a stirred solution of the product from step 6 above (AAT-9) (200 mg, 1 Eq, 544 μmol) in DCM (8 mL) was added TMSI (436 mg, 4 Eq, 2.18 mmol) dropwise at rt under nitrogen atmosphere. The resulting mixture was stirred for 2 h at rt under nitrogen atmosphere. The mixture was basified to pH 8 with sat. NaHCO₃ (aq.). The resulting mixture was diluted with water and extracted with DCM/MeOH (10/1) (3×50 mL). The combined organic layers were washed with brine (2×50 mL), dried (Na₂SO₄). After filtration, the filtrate was concentrated in vacuo to afford the sub-title compound (AAT-10) (140 mg, 523 μmol, 96%) as a brown solid. m/z 268.2 (M+H)⁺ (ES+).

Step 8: (1r,3r)-3-(3-Bromophenyl)-3-((4-methyl-4H-1,2,4-triazol-3-yl)methyl)cyclobutane-1-carbonitrile (AAT-11). To a stirred solution of the product from step 7 above (AAT-10) (720 mg, 1 Eq, 2.69 mmol) and tert-butyl nitrite (833 mg, 3 Eq, 8.08 mmol) in MeCN (15 mL) was added CuBr (580 mg, 1.5 Eq, 4.04 mmol) at rt under nitrogen atmosphere. The resulting mixture was stirred for 2 h at 50° C. under nitrogen atmosphere. The mixture was allowed to cool down to rt and concentrated in vacuo. The residue was purified by reverse flash chromatography with the following conditions: (column, C18 silica gel; mobile phase, MeCN in water (0.1% FA), 10% to 80% gradient in 20 min; detector, UV 254/220 nm) to afford in the sub-title compound (AAT-11) (430 mg, 48%) as a white solid. m/z 331.0/333.0 (M+H)⁺ (ES+).

Step 9: 3-((1r,3r)-3-Cyano-1-((4-methyl-4H-1,2,4-triazol-3-yl)methyl)cyclobutyl)benzoic acid (AAT-12). To a stirred solution of the product from step 8 above (AAT-11) (100 mg, 1 Eq, 302 μmol) and oxalic acid (54 mg, 2 Eq, 604 μmol) in DMF (6 mL) were added DIPEA (59 mg, 1.5 Eq, 453 μmol), acetic anhydride (62 mg, 2 Eq, 604 μmol), Pd(OAc)₂ (10 mg, 0.15 Eq, 45 μmol) and XantPhos (52 mg, 0.3 Eq, 91 μmol) at rt under nitrogen atmosphere. The resulting mixture was stirred for 4 h at 100° C. under nitrogen atmosphere. The mixture was allowed to cool down to rt. The residue was purified by reverse flash chromatography with the following conditions (column, C18 silica gel; mobile phase, MeCN in water (0.1% FA), 10% to 80% gradient in 20 min; detector, UV 254/220 nm) to afford the sub-title compound (AAT-12) (70 mg, 236 μmol, 78%) as a white solid. m/z 297.1 (M+H)⁺ (ES+).

Step 10: 3-((1r,3r)-3-Cyano-1-((4-methyl-4H-1,2,4-triazol-3-yl)methyl)cyclobutyl)-N-(2-hydroxy-3-(trifluoromethyl)phenyl)benzamide (AAT-13). To a stirred solution of the product from step 9 above (AAT-12) (110 mg, 1 Eq, 371 μmol) and 2-amino-6-(trifluoromethyl) phenol (K-1) (230 mg, 3.5 Eq, 1.30 mmol) in pyridine (10 mL) was added EDCI (142 mg, 2 Eq, 742 μmol) at rt. The resulting mixture was stirred for 2 h at 80° C. The mixture was allowed to cool down to rt and concentrated in vacuo. The residue was purified by reverse flash chromatography with the following conditions: (column, C18 silica gel; mobile phase, MeCN in water (0.1% FA), 10% to 80% gradient in 20 min; detector, UV 254/220 nm) to afford the sub-title compound (AAT-13) (30 mg, 66 μmol, 18%) as a green solid. m/z 456.2 (M+H)⁺ (ES+).

Step 11: (1r,3r)-3-((4-Methyl-4H-1,2,4-triazol-3-yl)methyl)-3-(3-(7-(trifluoromethyl) benzo[d]oxazol-2-yl)phenyl)cyclobutane-1-carbonitrile (AAT-14). A mixture of PPh₃ (35 mg, 3 Eq, 132 μmol) and DDQ (30 mg, 3 Eq, 132 μmol) in toluene (2 mL) was stirred for 30 min at rt under nitrogen atmosphere. To the above mixture was added the product from step 10 above (AAT-13) (20 mg, 1 Eq, 44 μmol). The resulting mixture was stirred for additional overnight at 110 degrees C. The mixture was allowed to cool down to rt and concentrated. The residue was purified by Prep-TLC with DCM/MeOH (10/1) and Prep-HPLC with the following conditions: (Column: XBridge Prep OBD C18 Column, 30*150 mm, 5 μm; Mobile Phase A: Water (0.1% NH₄HCO₃), Mobile Phase B: MeCN; Flow rate: 60 mL/min; Gradient: 48% B to 62% B in 8 min; Wave Length: 254/220 nm; RT: 5.73). The product-containing fractions were combined and evaporated partially in vacuo and lyophilized overnight to afford the title compound (AAT-14) (1.9 mg, 4.3 μmol, 10%) as a white solid. m/z 438.1 (M+H)⁺ (ES+). ¹H NMR (400 MHz, Methanol-d4) δ 8.21-8.14 (m, 2H), 8.03 (d, J=8.0 Hz, 1H), 7.96 (t, J=1.8 Hz, 1H), 7.74 (d, J=7.8 Hz, 1H), 7.64-7.54 (m, 2H), 7.37-3.31 (m, 1H), 3.50 (s, 2H), 3.28-3.20 (m, 1H), 3.09 (d, J=8.1 Hz, 4H), 2.87 (s, 3H).

### Example 100: Synthesis of (1S,2S)-2-(((2-(3-(1-Methyl-4-(4-methyl-4H-1,2,4-triazol-3-yl)-1H-pyrazol-5-yl)phenyl)-7-(trifluoromethyl)benzo[d]oxazol-5-yl)methyl)amino)cyclopentan-1-ol (AAU-6)

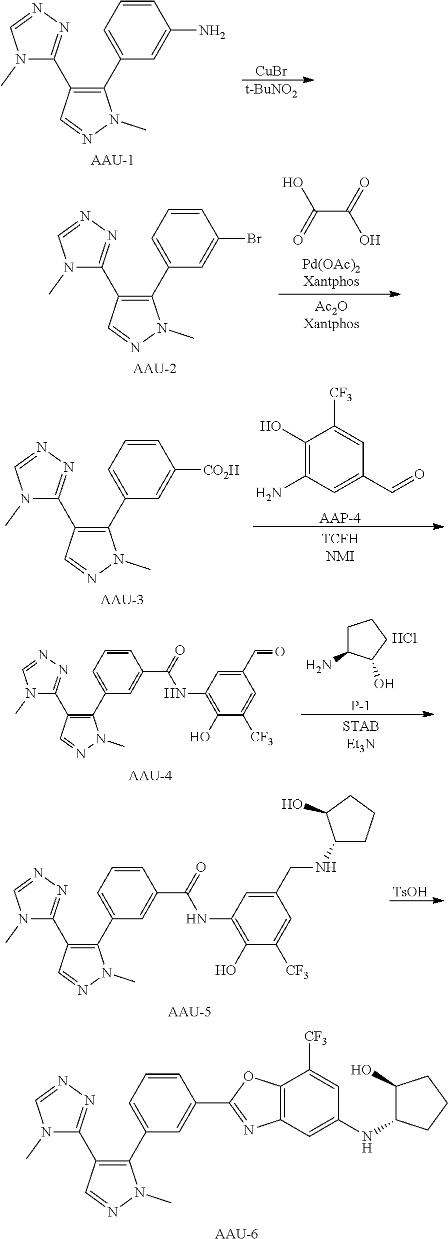

Step 1: 3-(5-(3-Bromophenyl)-1-methyl-1H-pyrazol-4-yl)-4-methyl-4H-1,2,4-triazole (AAU-2). Into a 100-mL round-bottom flask purged and maintained with an inert atmosphere of nitrogen, was placed CuBr (992 mg, 1.5 Eq, 6.91 mmol) and tert-butyl nitrite (122 mg, 6 Eq, 1.18 mmol) in DMSO (0.5 mL) and MeCN (18 mL) at 0° C. The resulting mixture was stirred for 0.5 h at 0° C. under nitrogen atmosphere. 3-(1-Methyl-4-(4-methyl-4H-1,2,4-triazol-3-yl)-1H-pyrazol-5-yl) aniline (AAU-1) (1.17 g, 1 Eq, 4.61 mmol) was added at rt under nitrogen atmosphere. The resulting mixture was stirred for 1 h at rt under nitrogen atmosphere. The resulting mixture was diluted with water and extracted with DCM (3×100 mL). The combined organic layers were washed with brine (2×100 mL), dried (Na₂SO₄). After filtration, the filtrate was concentrated in vacuo. The crude product was purified by reverse flash column chromatography with the following conditions: Column, C18; mobile phase, Water (0.1% NH₄HCO₃) and MeCN (0% MeCN up to 50% in 10 min); Detector, UV 254/220 nm. This resulted in the sub-title compound (AAU-2) (1.2 g, 3.79 mmol, 82%) as a yellow solid. m/z 318.0/320.0 (M+H)⁺ (ES+).

Step 2: 3-(1-Methyl-4-(4-methyl-4H-1,2,4-triazol-3-yl)-1H-pyrazol-5-yl)benzoic acid (AAU-3). Into a 100-mL round-bottom flask purged and maintained with an inert atmosphere of nitrogen, was placed the product from step 1 above (AAU-2) (600 mg, 1 Eq, 1.89 mmol) and oxalic acid (255 mg, 1.5 Eq, 2.83 mmol) in DMF (30 mL), then XantPhos (480 mg, 0.44 Eq, 830 μmol) and DIPEA (366 mg, 1.5 Eq, 2.83 mmol) and Pd(OAc)₂ (42 mg, 0.1 Eq, 189 μmol) were added at rt under nitrogen atmosphere. The resulting mixture was stirred for overnight at 100° C. under nitrogen atmosphere. The mixture was cooled to rt. The resulting mixture was diluted with water and extracted with DCM (3×80 mL). The combined organic layers were washed with brine (2×80 mL), dried (Na₂SO₄). After filtration, the filtrate was concentrated in vacuo. The crude product was purified by reverse flash column chromatography with the following conditions: Column, C18; mobile phase, Water (0.1% FA) and MeCN (0% MeCN up to 40% in 10 min); Detector, UV 254/220 nm. This resulted in the sub-title compound (AAU-3) (400 mg, 1.41 mmol, 75%) as a white solid. m/z 284.1 (M+H)⁺ (ES+).

Step 3: N-(5-Formyl-2-hydroxy-3-(trifluoromethyl)phenyl)-3-(1-methyl-4-(4-methyl-4H-1,2,4-triazol-3-yl)-1H-pyrazol-5-yl)benzamide (AAU-4). Into a 250-mL round-bottom flask purged and maintained with an inert atmosphere of nitrogen, was placed the product from step 2 above (AAU-3) (200 mg, 1 Eq, 706 μmol) and intermediate (AAP-4) (174 mg, 1.2 Eq, 847 μmol) in MeCN (10 mL), then N,N,N′,N′-tetramethylchloroformamidinium-hexafluorophosphate (594 mg, 3 Eq, 2.12 mmol) and 1-methylimidazole (869 mg, 15 Eq, 10.6 mmol) were added at rt under nitrogen atmosphere. The resulting mixture was stirred for 2 h at rt under nitrogen atmosphere. The resulting mixture was diluted with water and extracted with DCM (3×50 mL). The combined organic layers were washed with brine (2×50 mL), dried (Na₂SO₄). After filtration, the filtrate was concentrated in vacuo. The crude product was purified by reverse flash column chromatography with the following conditions: Column, C18; mobile phase, Water (0.1% NH₄HCO₃) and MeCN (0% MeCN up to 50% in 20 min); Detector, UV 254/220 nm. This resulted in the sub-title compound (AAU-4) (130 mg, 276 μmol, 39%) as a white solid. m/z 471.1 (M+H)⁺ (ES+).

Step 4: N-(2-Hydroxy-5-((((1S,2S)-2-hydroxycyclopentyl)amino)methyl)-3-(trifluoromethyl) phenyl)-3-(1-methyl-4-(4-methyl-4H-1,2,4-triazol-3-yl)-1H-pyrazol-5-yl)benzamide (AAU-5). Into a 50-mL round-bottom flask purged and maintained with an inert atmosphere of nitrogen, was placed the product from step 3 above (AAU-4) (100 mg, 1 Eq, 213 μmol) and (1S,2S)-2-aminocyclopentan-1-ol, HCl (P-1) (117 mg, 4 Eq, 852 μmol) and Et₃N (65 mg, 3 Eq, 639 μmol) in DCM (15 mL). The resulting mixture was stirred for 1 h at rt, and then NaBH(OAc)₃ (90 mg, 2 Eq, 426 μmol) were added at rt. The resulting mixture was stirred for 2 h at rt. The resulting mixture was diluted with water and extracted with DCM (3×20 mL). The combined organic layers were washed with brine (2×20 mL), dried (Na₂SO₄). After filtration, the filtrate was concentrated in vacuo. The crude product was purified by reverse flash column chromatography with the following conditions: Column, C18; mobile phase, Water (0.1% NH₄HCO₃) and MeCN (0% MeCN up to 50% in 20 min); Detector, UV 254/220 nm. This resulted in the sub-title compound (AAU-5) (60 mg, 108 μmol, 51%) as a white solid. m/z 556.2 (M+H)⁺ (ES+).

Step 5: (1S,2S)-2-(((2-(3-(1-Methyl-4-(4-methyl-4H-1,2,4-triazol-3-yl)-1H-pyrazol-5-yl) phenyl)-7-(trifluoromethyl)benzo[d]oxazol-5-yl)methyl)amino)cyclopentan-1-ol (AAU-6). Into a 20-mL vial purged and maintained with an inert atmosphere of nitrogen, was placed the product from step 4 above (AAU-5) (30 mg, 1 Eq, 54 μmol) and p-TsOH-H₂O (19 mg, 2 Eq, 108 μmol) in NMP (3 mL), The resulting mixture was stirred for overnight at 120° C. under nitrogen atmosphere. The mixture was cooled to rt and concentrated in vacuo. The crude product was purified by Prep-HPLC with the following conditions Gradient: (Column: XBridge Prep OBD C18 Column, 30×150 mm, 5 μm; Mobile Phase A: Water (0.1% NH₄HCO₃), Mobile Phase B: MeCN; Flow rate: 60 mL/min; Gradient: 46% B to 63% B in 8 min; Wave Length: 254/220 nm; RT: 7.8). This resulted in the title compound (AAU-6) (100 mg, 186 μmol, 37%) as a white solid. m/z 538.3 (M+H)⁺ (ES+). ¹H NMR (400 MHz, Methanol-d4) δ 8.46 (s, 1H), 8.38 (d, J=7.8 Hz, 1H), 8.23 (t, J=1.8 Hz, 1H), 8.05 (s, 1H), 7.94 (s, 1H), 7.82-7.71 (m, 3H), 4.05-3.95 (m, 6H), 3.50 (s, 3H), 2.96-2.88 (m, 1H), 2.10-1.98 (m, 2H), 1.76-1.70 (m, 2H), 1.62-1.92 (m, 1H), 1.49-1.38 (m, 1H).

### Example 101: Synthesis of (1R,2R)-2-(((2-(3-(1-Methyl-4-(4-methyl-4H-1,2,4-triazol-3-yl)-1H-pyrazol-5-yl)phenyl)-7-(trifluoromethyl)benzo[d]oxazol-5-yl) methyl)amino)cyclopentan-1-ol (AAV-2)

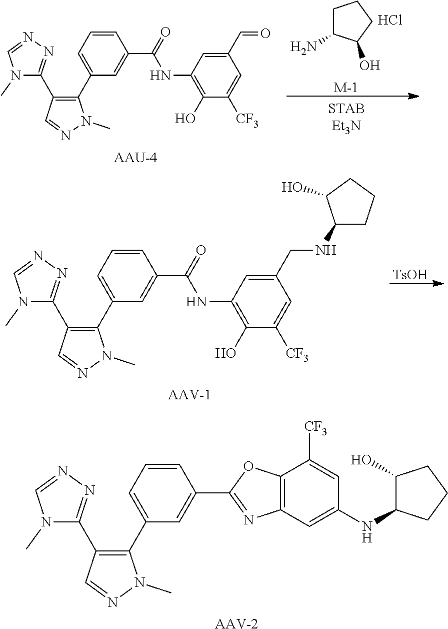

Step 1: N-(2-Hydroxy-5-((((1R,2R)-2-hydroxycyclopentyl)amino)methyl)-3-(trifluoromethyl) phenyl)-3-(1-methyl-4-(4-methyl-4H-1,2,4-triazol-3-yl)-1H-pyrazol-5-yl)benzamide (AAV-1). Into a 40-mL vial purged and maintained with an inert atmosphere of nitrogen, was placed intermediate (AAU-4) (50 mg, 1 Eq, 106 μmol) and (1R,2R)-2-aminocyclopentan-1-ol, HCl (M-1) (43 mg, 4 Eq, 424 μmol) and Et₃N (32 mg, 3 Eq, 318 μmol) in DCM (15 mL). The resulting mixture was stirred for 1 h at rt, and then NaBH(OAc)₃ (45 mg, 2 Eq, 212 μmol) were added at rt. The resulting mixture was stirred for 2 h at rt. The resulting mixture was diluted with water and extracted with DCM (3×20 mL). The combined organic layers were washed with brine (2×20 mL), dried (Na₂SO₄). After filtration, the filtrate was concentrated in vacuo. The crude product was purified by reverse flash column chromatography with the following conditions: Column, C18; mobile phase, Water (0.1% NH₄HCO₃) and MeCN (0% MeCN up to 60% in 20 min); Detector, UV 254/220 nm. This resulted in the sub-title compound (AAV-1) (50 mg, 90 μmol, 85%) as a white solid. m/z 556.2 (M+H)⁺ (ES+).

Step 2: (1R,2R)-2-(((2-(3-(1-Methyl-4-(4-methyl-4H-1,2,4-triazol-3-yl)-1H-pyrazol-5-yl) phenyl)-7-(trifluoromethyl)benzo[d]oxazol-5-yl)methyl)amino)cyclopentan-1-ol (AAV-2). Into a 20-mL vial purged and maintained with an inert atmosphere of nitrogen, was placed the product from step 1 above (AAV-1) (37 mg, 1 Eq, 67 μmol) and pTsOH-H₂O (23 mg, 2 Eq, 134 μmol) in NMP (3 mL). The resulting mixture was stirred for overnight at 120° C. under nitrogen atmosphere. The mixture was cooled to rt and concentrated in vacuo. The crude product was purified by Prep-HPLC with the following conditions (Column: XBridge Prep C18 OBD Column, 30×100 mm, 5 μm; Mobile Phase A: Water (0.1% NH₄HCO₃+0.1% NH₃·H₂O), Mobile Phase B: MeCN; Flow rate: 60 mL/min; Gradient: 23% B to 43% B in 10 min; Wave Length: 254/220 nm; RT: 9.67). This resulted in the title compound (AAV-2) (2.4 mg, 4.5 μmol, 6.6%) as a white solid. m/z 538.1 (M+H)⁺ (ES+). ¹H NMR (400 MHz, Methanol-d4) δ 8.46 (s, 1H), 8.38 (d, J=7.8 Hz, 1H), 8.23 (d, J=2.2 Hz, 1H), 8.05 (s, 1H), 7.95 (s, 1H), 7.82-7.70 (m, 3H), 4.05-3.97 (m, 6H), 3.50 (s, 3H), 2.96-2.88 (m, 1H), 2.09-1.96 (m, 2H), 1.77-1.67 (m, 2H), 1.60-1.54 (m, 1H), 1.50-1.39 (m, 1H).

### Example 102: Synthesis of 3′-(5-((((1R,2S)-2-Hydroxycyclopentyl)amino)methyl)-7-(trifluoromethyl)-1H-benzo[d]imidazol-2-yl)-2-(4-methyl-4H-1,2,4-triazol-3-yl)-[1,1′-biphenyl]-4-carbonitrile (AAW-5)

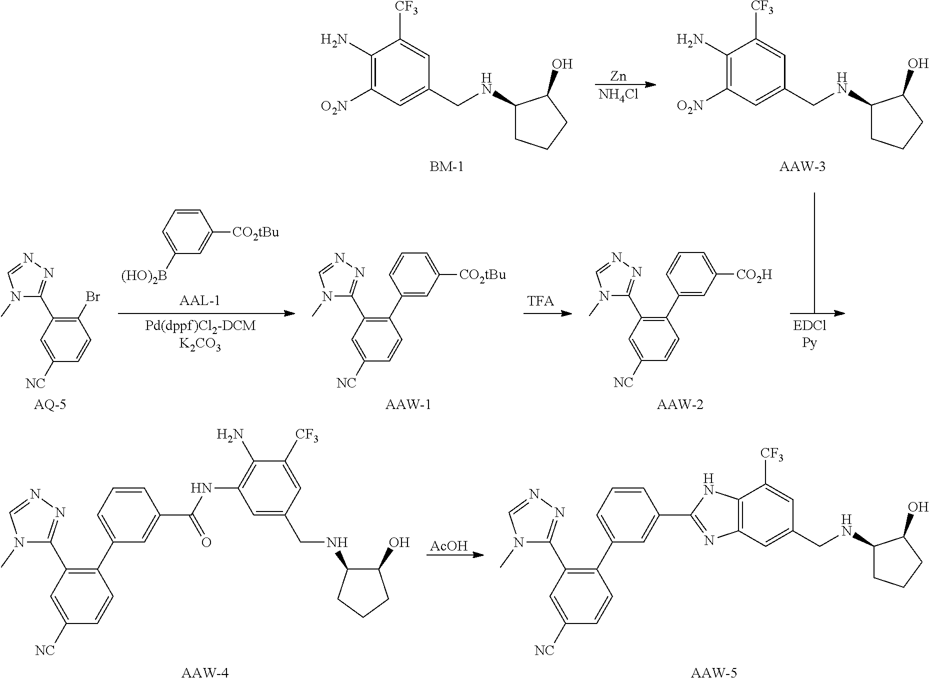

Step 1: tert-Butyl 4′-cyano-2′-(4-methyl-1,2,4-triazol-3-yl)-[1,1′-biphenyl]-3-carboxylate (AAW-1). To a solution of intermediate (AQ-5) (160 mg, 1 Eq, 608 μmol) and intermediate (AAL-1) (135 mg, 1 Eq, 608 μmol) in dioxane (5 mL) and water (1 mL) were added K₂CO₃ (252 mg, 3 Eq, 1.82 mmol) and Pd(dppf)Cl₂-DCM (45 mg, 0.1 Eq, 61 μmol) at rt under nitrogen atmosphere. After stirring for 3 hours at 80° C. under nitrogen atmosphere, the mixture was allowed to cool down to rt. The resulting mixture was diluted with water and extracted with EtOAc (3×20 mL). The combined organic layers were washed with brine (2×20 mL), dried (Na₂SO₄). After filtration, the filtrate was concentrated in vacuo. The residue was purified by TLC with DCM/MeOH (12/1) to afford the sub-title compound (AAW-1) (150 mg, 417 μmol, 68%) as a yellow solid. m/z 361.2 (M+H)⁺ (ES+).

Step 2: 4′-Cyano-2′-(4-methyl-1,2,4-triazol-3-yl)-[1,1′-biphenyl]-3-carboxylic acid (AAW-2). To a stirred solution of the product from step 1 above (AAW-1) (145 mg, 1 Eq, 402 μmol) in DCM (3 mL) was added TFA (1 mL) dropwise at rt. The resulting mixture was stirred for 1 hour at rt. The resulting mixture was concentrated in vacuo. The crude product was used in the next step directly without further purification. m/z 305.1 (M+H)⁺ (ES+).

Step 3: (1S,2R)-2-((3,4-Diamino-5-(trifluoromethyl)benzyl)amino)cyclopentan-1-ol (AAW-3). Into a 50-mL round-bottom flask purged and maintained with an inert atmosphere of nitrogen, was placed intermediate (BM-1) (300 mg, 1 Eq, 940 μmol) and NH₄Cl (503 mg, 10 Eq, 9.40 mmol) in MeOH (20 mL) at rt, then zinc (307 mg, 5 Eq, 4.70 mmol) was added at rt. The resulting solution was stirred for 2 h at rt. The resulting mixture was filtered the filter cake was washed with MeOH (3×15 mL). The filtrate was concentrated in vacuo. The crude product was purified by reverse flash column chromatography with the following conditions: Column, C18; mobile phase, Water (0.1% TFA) and MeCN (10% MeCN up to 50% in 20 min); Detector, UV 254/220 nm. The product-containing fractions were combined and concentrated in vacuo. This resulted in the sub-title compound (AAW-3) (156 mg, 538 μmol, 57%) as yellow oil. m/z 290.1 (M+H)⁺ (ES+).

Step 4: N-[2-Amino-5-({[(1R,2S)-2-hydroxycyclopentyl]amino}methyl)-3-(trifluoromethyl) phenyl]-4′-cyano-2′-(4-methyl-1,2,4-triazol-3-yl)-[1,1′-biphenyl]-3-carboxamide (AAW-4). To a stirred solution of the product from step 2 above (AAW-2) (20 mg, 1 Eq, 66 μmol) and the product from step 3 above (AAW-3) (19 mg, 1 Eq, 66 μmol) in pyridine (1.5 mL) was added EDCI (26 mg, 2 Eq, 132 mmol) at rt. The resulting mixture was stirred for 2 hours at 60° C. The mixture was allowed to cool down to rt and concentrated in vacuo. The crude product was purified by Prep-HPLC with the following conditions (Column: XBridge Prep OBD C18 Column, 30*150 mm, 5 μm; Mobile Phase A: Water (0.1% NH₄HCO₃), Mobile Phase B: MeCN; Flow rate: 60 mL/min; Gradient: 28% B to 38% B in 8 min; Wave Length: 254/220 nm) to afford the title compound (AAW-4) (2.9 mg, 5.0 μmol, 7.7%) as a white solid. m/z 576.2 (M+H)⁺ (ES+).

Step 5: 3′-[6-({[(1R,2S)-2-Hydroxycyclopentyl]amino}methyl)-4-(trifluoromethyl)-3H-1,3-benzodiazol-2-yl]-2-(4-methyl-1,2,4-triazol-3-yl)-[1,1′-biphenyl]-4-carbonitrile (AAW-5). A solution of the product from step 4 above (AAW-4) (25 mg, 1 Eq, 43 μmol) in AcOH (1.5 mL) was stirred for 2 hours at 80° C. under nitrogen atmosphere. The resulting mixture was allowed to cool down to rt and then concentrated in vacuo. The crude product was purified by Prep-HPLC with the following conditions (Column: XBridge Prep OBD C18 Column, 30*150 mm, 5 μm; Mobile Phase A: Water (0.1% NH₄HCO₃), Mobile Phase B: MeCN; Flow rate: 60 mL/min; Gradient: 30% B to 43% B in 9 min; Wave Length: 254/220 nm) to afford the title compound (AAW-5) (6.3 mg, 11 μmol, 26%) as a white solid. m/z 558.0 (M+H)⁺ (ES+). ¹H NMR (400 MHz, DMSO-d6) δ 13.36 (s, 1H), 8.45 (s, 1H), 8.34-8.08 (m, 4H), 7.95 (d, J=8.1 Hz, 1H), 7.80 (s, 1H), 7.57-7.51 (m, 2H), 7.15 (d, J=7.7 Hz, 1H), 4.04-3.85 (m, 3H), 3.20 (s, 3H), 2.88-2.79 (m, 1H), 1.78-1.52 (m, 4H), 1.51-1.37 (m, 2H).

### Example 103: Synthesis of 6-{[(3S)-3-Methylpiperidin-1-yl]methyl}-2-{3-[(1r,3s)-3-me thyl-1-(4-methyl-1,2,4-triazol-3-yl)cyclobutyl]phenyl}-4-(trifluorometh yl)-3H-1,3-benzodiazole (AAX-12)

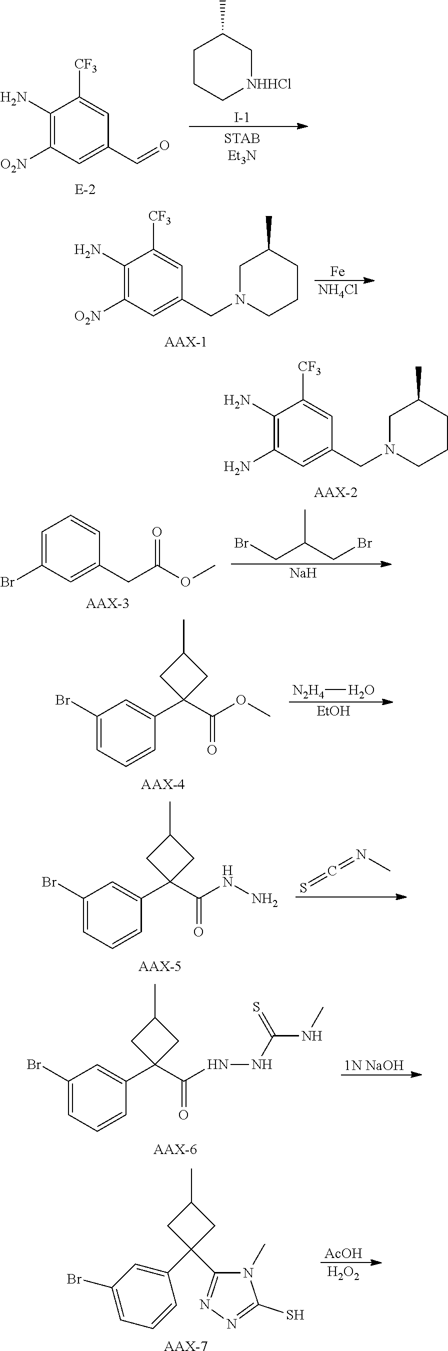

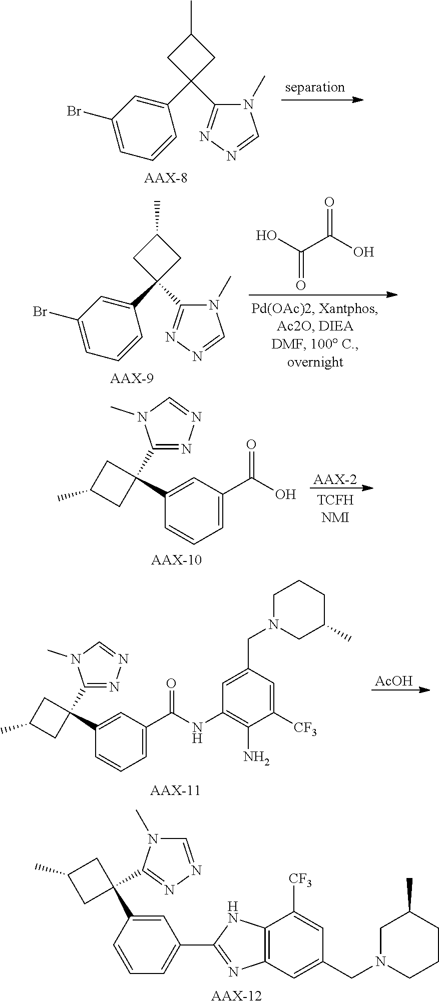

Step 1: 4-{[(3S)-3-Methylpiperidin-1-yl]methyl}-2-nitro-6-(trifluoromethyl)aniline (AAX-1). To a stirred solution of intermediate (E-2) (200 mg, 1 Eq, 854 μmol) and intermediate (I-1) (169 PP-673,C3 mg, 2 Eq, 1.71 mmol) in DCM (15 mL) was added Et₃N (173 mg, 2 Eq, 1.71 mmol) and NaBH(OAc)₃ (362 mg, 2 Eq, 1.71 mmol) at rt. The resulting mixture was stirred overnight at 40° C. under nitrogen atmosphere. The mixture was cooled to rt and diluted with water and extracted with DCM (3×50 mL). The combined organic layers were washed with brine (2×50 mL), dried (Na₂SO₄). After filtration, the filtrate was concentrated in vacuo. The crude product was purified by reverse flash column chromatography with the following conditions: Column, C18; mobile phase, Water (0.1% NH₄HCO₃) and MeCN (10% MeCN up to 40% in 10 min); Detector, UV 254/220 nm. The product-containing fractions were combined and concentrated in vacuo. This resulted in the sub-title compound (AAX-1) (200 mg, 631 μmol, 74%) as a white solid. m/z 318.1 (M+H)⁺ (ES+).

Step 2: 5-{[(3S)-3-Methylpiperidin-1-yl]methyl}-3-(trifluoromethyl)benzene-1,2-diamine (AAX-2). To a stirred solution of the product from step 1 above (AAX-1) (120 mg, 1 Eq, 378 μmol) and NH₄Cl (202 mg, 10 Eq, 3.78 mmol) in EtOH (8 mL) and water (2 mL) was added Fe powder (106 mg, 5 Eq, 1.89 mmol) at rt. The resulting mixture was stirred for 2 hours at 80° C. The mixture was cooled to rt and filtered the filter cake was washed with EtOH (3×15 mL). The filtrate was concentrated in vacuo. The residue was purified by Prep-TLC with DCM/MeOH (10/1) to afford the sub-title compound (AAX-2) (90 mg, 313 μmol, 83%) as a yellow solid. m/z 288.2 (M+H)⁺ (ES+).

Step 3: Methyl 1-(3-bromophenyl)-3-methylcyclobutane-1-carboxylate (AAX-4). Into a 500 mL round-bottom flask were added methyl 2-(3-bromophenyl)acetate (AAX-3) (4.00 g, 1 Eq, 17.5 mmol) and 1,3-dibromo-2-methylpropane (3.80 g, 1 Eq, 17.5 mmol) in DMF (60 mL) at rt. To the above mixture was added NaH (1.40 g, 60% Wt, 2 Eq, 34.9 mmol) over 3 min at 0° C. The resulting mixture was stirred overnight at rt. The reaction was quenched with sat. NH₄C1 (aq.) at 0° C. The resulting mixture was diluted with water and extracted with DCM (3×200 mL). The combined organic layers were washed with brine (2×200 mL), dried (Na₂SO₄). After filtration, the filtrate was concentrated in vacuo. The residue purified by silica gel column chromatography with petroleum ether/EtOAc (8/1). This resulted in the sub-title compound (AAX-4) (3.8 g, 13.5 mmol, 77%) as a yellow solid. m/z 283.0/285.0 (M+H)⁺ (ES+).

Step 4: 1-(3-Bromophenyl)-3-methylcyclobutane-1-carbohydrazide (AAX-5). Into a 250 mL round-bottom flask were added the product from step 3 above (AAX-4) (1.70 g, 1 Eq, 6.00 mmol) in EtOH (25 mL) at rt. To the above mixture was added hydrazine (20 mL) dropwise over 5 min at rt. The resulting mixture was stirred overnight at 80° C. The mixture was cooled to rt. The resulting mixture was diluted with water and extracted with EtOAc (3×200 mL). The combined organic layers were washed with brine (2×200 mL), dried (Na₂SO₄). After filtration, the filtrate was concentrated in vacuo to afford the sub-title compound (AAX-5) (1.6 g, 5.65 mmol, 94%) as yellow oil. m/z 283.0/285.0 (M+H)⁺ (ES+).

Step 5: 1-(3-Bromophenyl)-3-methyl-N-[(methylcarbamothioyl)amino]cyclobutane-1-carboxamide (AAX-6). Into a 100 mL round-bottom flask were added the product from step 4 above (AAX-5) (1.60 g, 1 Eq, 5.65 mmol) and methyl isothiocyanate (1.24 g, 3 Eq, 17.0 mmol) in THF (30 mL) at rt. The resulting mixture was stirred for 3 h at 80° C. The mixture was allowed to cool down to rt. The resulting mixture was concentrated in vacuo. This resulted in the sub-title compound (AAX-6) (1.4 g, 3.94 mmol, 70%) as colorless oil. m/z 356.0/368.0 (M+H)⁺ (ES+).

Step 6: 5-[1-(3-Bromophenyl)-3-methylcyclobutyl]-4-methyl-1,2,4-triazole-3-thiol (AAX-7). Into a 250 mL round-bottom flask were added the product from step 5 above (AAX-6) (1.60 g, 1 Eq, 4.49 mmol) in THF (30 mL) at rt. To the above mixture was added NaOH (690 mg, 3.8 Eq, 17.2 mmol) in water (5 mL) dropwise at rt. The resulting mixture was stirred overnight at 60° C. The mixture was allowed to cool down to rt. The resulting mixture was diluted with water (100 mL). The mixture was acidified to pH 5 with HCl (aq., 1 molar). The resulting mixture was diluted with water and extracted with EtOAc (3×100 mL). The combined organic layers were washed with brine (2×100 mL), dried (Na₂SO₄). After filtration, the filtrate was concentrated in vacuo to afford the sub-title compound (AAX-7) (1.3 g, 3.86 mmol, 86%) as yellow oil. m/z 338.0/340.0 (M+H)⁺ (ES+).

Step 7: 3-[1-(3-Bromophenyl)-3-methylcyclobutyl]-4-methyl-1,2,4-triazole (AAX-8). Into a 20 mL sealed tube were added the product from step 6 above (AAX-7) (500 mg, 1 Eq, 1.48 mmol) and AcOH (178 mg, 2 Eq, 2.96 mmol) in DCM (8 mL) at rt. To the above mixture was added hydrogen peroxide (251 mg, 30% Wt, 5 Eq, 7.39 mmol) dropwise over 3 min at 0° C. The resulting mixture was stirred for additional 3 h at rt. The resulting mixture was concentrated in vacuo. The residue was applied on a silica gel column chromatography with petroleum ether/EtOAc (5/1) to afford the sub-title compound (AAX-8) (300 mg, 983 μmol, 66%) as yellow oil. m/z 306.1/308.1 (M+H)⁺ (ES+).

Step 8: 4-Methyl-3-[(1r,3s)-1-(3-bromophenyl)-3-methylcyclobutyl]-1,2,4-triazole (AAX-9). The crude product the product from step 7 above (AAX-8) (300 mg, 1 Eq, 983 μmol) was purified by reverse flash column chromatography with the following conditions: Column, C18; mobile phase, Water (0.05% TFA) and MeCN (10% MeCN up to 40% in 30 min); Detector, UV 254/220 nm. The product-containing fractions were combined and concentrated in vacuo. This resulted in the sub-title compound (AAX-9) (170 mg, 557 μmol, 57%) as yellow oil. m/z 306.1/308.1 (M+H)⁺ (ES+).

Step 9: 3-((1s,3s)-3-Methyl-1-(4-methyl-4H-1,2,4-triazol-3-yl)cyclobutyl)benzoic acid (AAX-10). To a stirred solution of the product from step 8 above (60 mg, 1 Eq, 196 μmol) and oxalic acid (26 mg, 1.5 Eq, 294 μmol) in DMF (3 mL) was added acetic anhydride (30 mg, 1.5 Eq, 294 μmol), DIPEA (38 mg, 1.5 Eq, 294 μmol), Pd(AcO)₂ (4.4 mg, 0.1 Eq, 20 μmol) and XantPhos (23 mg, 0.2 Eq, 39 μmol) at rt under nitrogen atmosphere. The resulting mixture was stirred for overnight at 100° C. under nitrogen atmosphere. The crude product was cooled to rt and purified by reverse flash column chromatography with the following conditions: Column, C18; mobile phase, Water (0.1% NH₄HCO₃) and MeCN (0% MeCN up to 30% in 20 min); Detector, UV 254/220 nm. This resulted in the sub-title compound (AAX-10) (35 mg, 129 μmol, 66%) as a white solid. m/z 272.1 (M+H)⁺ (ES+).

Step 10: N-(2-Amino-5-{[(3S)-3-methylpiperidin-1-yl]methyl}-3-(trifluoromethyl) phenyl)-3-[(1r,3s)-3-methyl-1-(4-methyl-1,2,4-triazol-3-yl) cyclobutyl]benzamide (AAX-11). To a stirred solution of the product from step 2 above (AAX-2) (30 mg, 1 Eq, 104 μmol) and the product from step 9 (AAX-10) (28 mg, 1 Eq, 104 μmol) in MeCN (4 mL) were added N,N,N′,N′-tetramethylchloroformamidinium-hexafluorophosphate (88 mg, 3 Eq, 312 μmol) and 1-methylimidazole (86 mg, 10 Eq, 1.04 mmol) at 0° C. The resulting mixture was stirred for 3 hours at rt. The crude product was purified by reverse flash column chromatography with the following conditions: Column, C18; mobile phase, Water (0.1% FA) and MeCN (20% MeCN up to 55% in 10 min); Detector, UV 254/220 nm). The product-containing fractions were combined and concentrated in vacuo. This resulted in the sub-title compound (AAX-11) (45 mg, 83 μmol, 80%) as a yellow solid. m/z 541.3 (M+H)⁺ (ES+).

Step 11: 6-{[(3S)-3-Methylpiperidin-1-yl]methyl}-2-{3-[(1r,3s)-3-methyl-1-(4-methyl-1,2,4-triazol-3-yl)cyclobutyl]phenyl}-4-(trifluoromethyl)-3H-1,3-benzodiazole (AAX-12). The product from step 9 above (AAX-11) (50 mg, 1 Eq, 92 μmol) was added in AcOH (5 mL) at rt. The resulting mixture was stirred for overnight at 80° C. under nitrogen atmosphere. The resulting mixture was cooled to rt and concentrated in vacuo. The crude product was purified by reverse flash column chromatography with the following conditions: Column, C18; mobile phase, Water (0.1% NH₄HCO₃) and MeCN (36% MeCN up to 68% in 10 min); Detector, UV 254/220 nm. The product-containing fractions were combined and concentrated in vacuo. The crude product was purified by Prep-HPLC with the following conditions: Column: XBridge Prep OBD C18 Column, 30×150 mm 5 um; Mobile Phase A: Water (0.1% NH₄HCO₃), Mobile Phase B: MeCN; Flow rate: 60 mL/min; Gradient: 47 B to 67 B in 8 min; Detector, UV 254/210 nm; RT: 7.67. The product-containing fractions were combined and evaporated partially in vacuo and lyophilized overnight to afford the title compound (AAX-12) (13.6 mg, 26 μmol, 28%) as a white solid. m/z 523.3 (M+H)⁺ (ES+). ¹H NMR (400 MHz, Methanol-d4) δ 8.34 (d, J=10.3 Hz, 2H), 8.10 (d, J=7.7 Hz, 1H), 7.83 (s, 1H), 7.60 (t, J=7.7 Hz, 2H), 7.50-7.45 (m, 1H), 3.73 (s, 2H), 3.44 (s, 3H), 3.10-3.03 (m, 2H), 2.97-2.85 (m, 2H), 2.70-2.60 (m, 3H), 2.06-1.97 (m, 1H), 1.78-1.62 (m, 5H), 1.20 (d, J=6.3 Hz, 3H), 0.89 (d, J=5.5 Hz, 4H).

### Example 104: Synthesis of 5-{[(3S)-3-Methylpiperidin-1-yl]methyl}-2-{3-[(1r,3s)-3-me thyl-1-(4-methyl-1,2,4-triazol-3-yl)cyclobutyl]phenyl}-7-(trifluorometh yl)-1,3-benzoxazole (AAY-2)

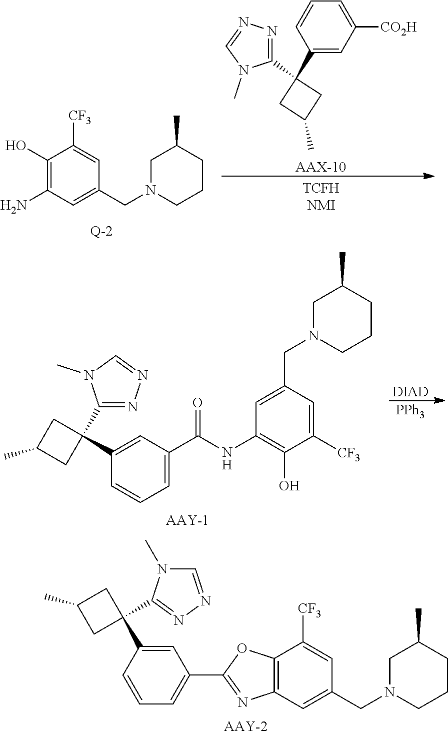

Step 1: N-(2-Hydroxy-5-{[(3S)-3-methylpiperidin-1-yl]methyl}-3-(trifluoromethyl)phenyl)-3-[(1r,3s)-3-methyl-1-(4-methyl-1,2,4-triazol-3-yl)cyclobutyl]benzamide (AAY-1). To a stirred solution of intermediate (AAX-10) (40 mg, 1 Eq, 147 μmol) and intermediate (Q-2) (43 mg, 1 Eq, 147 μmol) in MeCN (3 mL) was added N,N,N′,N′-tetramethylchloroforma midinium-hexafluorophosphate (124 mg, 3 Eq, 441 μmol) and 1-methylimidazole (121 mg, 10 Eq, 1.47 mmol) at 0° C. The resulting mixture was stirred for 3 hours at rt. The crude product was purified by reverse flash column chromatography with the following conditions: Column, C18; mobile phase, Water (0.1% FA) and MeCN (20% MeCN up to 55% in 10 min); Detector, UV 254/220 nm. The product-containing fractions were combined and concentrated in vacuo. This resulted in the sub-title compound (AAY-1) (30 mg, 55 μmol, 38%) as yellow oil. m/z 542.3 (M+H)⁺ (ES+).

Step 2: 5-{[(3S)-3-Methylpiperidin-1-yl]methyl}-2-{3-[(1r,3s)-3-methyl-1-(4-methyl-1,2,4-triazol-3-yl)cyclobutyl]phenyl}-7-(trifluoromethyl)-1,3-benzoxazole (AAY-2). To a stirred solution of PPh₃ (24 mg, 2 Eq, 92 μmol) and DIAD (19 mg, 2 Eq, 92 μmol) in THF (5 mL) was added the product from step 1 above (AAY-1) (25 mg, 1 Eq, 46 μmol) at 0° C. under nitrogen atmosphere. The resulting mixture was stirred for overnight at 60° C. under nitrogen atmosphere. The mixture was cooled to rt and concentrated in vacuo. The residue was purified by Prep-TLC with EtOAc/petroleum ether (1/1). The crude product was purified by Prep-HPLC with the following conditions: Column: XBridge Prep OBD C18 Column, 30×150 mm 5 um; Mobile Phase A: Water (0.11% NH₄HCO₃), Mobile Phase B: MeCN; Flow rate: 60 mL/min; Gradient: 72 B to 82 B in 8 min; Detector, UV 254/210 nm; RT: 5.68. The product-containing fractions were combined and evaporated partially in vacuo and lyophilized overnight to afford the title compound (AAY-2) (2.6 mg, 5.0 μmol, 11%) as a white solid. m/z 524.1 (M+H)⁺ (ES+). ¹H NMR (400 MHz, Methanol-d4) δ 8.36-8.29 (m, 2H), 8.22-8.18 (m, 1H), 8.01 (s, 1H), 7.76-7.64 (m, 3H), 3.75 (s, 2H), 3.44 (s, 3H), 3.07-2.98 (m, 2H), 2.90 (t, J=12.6 Hz, 2H), 2.70-2.60 (m, 3H), 2.07 (d, J=11.1 Hz, 1H), 1.72-1.62 (m, 5H), 1.21 (d, J=5.9 Hz, 3H), 1.01-0.85 (m, 4H).

### Example 105: Synthesis of (S)-2-(6-Cyclopropyl-4-(4-fluoro-2-(4-methyl-4H-1,2,4-triaz ol-3-yl)phenyl)pyridin-2-yl)-5-((3-methylpiperidin-1-yl)methyl)-7-(trifluoromethyl)-1H-benzo[d]imidazole (AAZ-10)

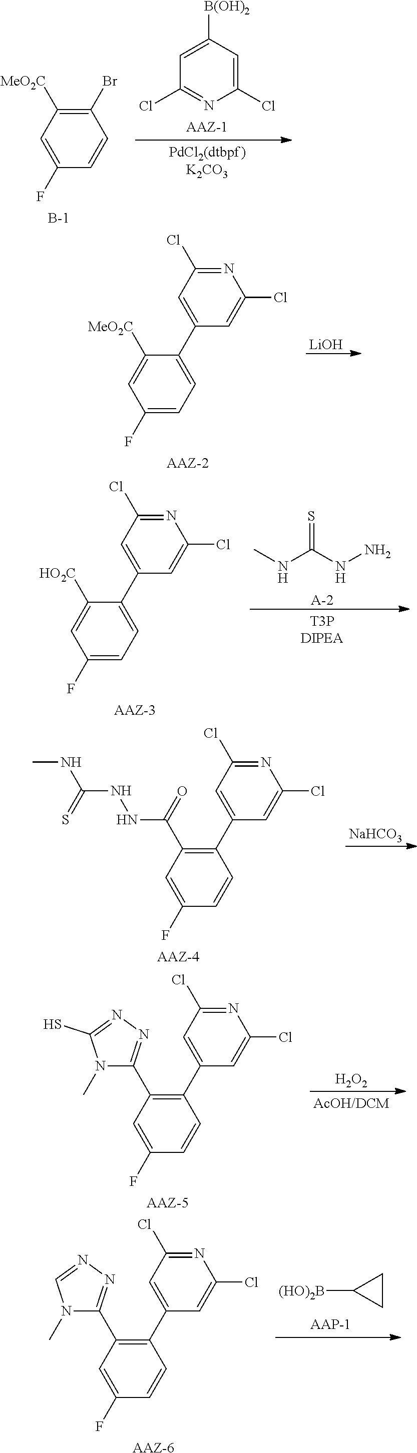

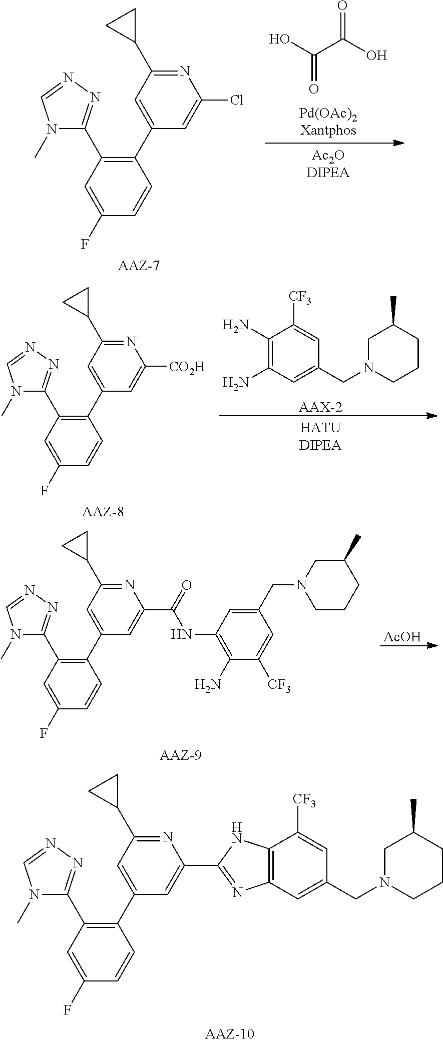

Step 1: Methyl 2-(2,6-dichloropyridin-4-yl)-5-fluorobenzoate (AAZ-2). Into a 500-mL round-bottom flask purged and maintained with an inert atmosphere of nitrogen, was placed intermedoate (B-1) (4.20 g, 1 Eq, 18.0 mmol) and 2,6-dichloropyridin-4-ylboronic acid (AAZ-1) (3.46 g, 1 Eq, 18.0 mmol) in 1,4-dioxane (150 mL) and water (30 mL), then Pd(DtBPF)Cl₂ (1.17 g, 0.1 Eq, 1.80 mmol) and K₂CO₃ (7.47 g, 3 Eq, 54.1 mmol) were added at rt. The resulting solution was stirred for overnight at 60° C. The mixture was cooled to rt, diluted with water and extracted with EtOAc (3×300 mL). The combined organic layers were washed with brine (2×300 mL), dried (Na₂SO₄). After filtration, the filtrate was concentrated in vacuo. The crude product was purified by reverse flash column chromatography with the following conditions: Column, C18; mobile phase, Water (0.1% NH₄HCO₃) and MeCN (26% MeCN up to 52% in 15 min); Detector, UV 254/220 nm. This resulted in the sub-title compound (AAZ-2) (2.5 g, 8.36 mmol, 46%) as a yellow solid. m/z 300.0/302.0 (M+H)⁺ (ES+).

Step 2: 2-(2,6-Dichloropyridin-4-yl)-5-fluorobenzoic acid (AAZ-3). Into a 250-mL round-bottom flask, was placed the product from step 1 above (AAZ-2) (2.50 g, 1 Eq, 8.33 mmol) in THF (100 mL) and water (25 mL), then LiOH (600 mg, 3 Eq, 25.0 mmol) was added at rt. The resulting solution was stirred for 16 h at 60° C. The mixture was cooled to rt, diluted with water and extracted with EtOAc (3×150 mL). The combined organic layers were washed with brine (2×150 mL), dried (Na₂SO₄). After filtration, the filtrate was concentrated in vacuo. This resulted in the sub-title compound (AAZ-3) (2.2 g, 7.72 mmol, 92%) as a brown yellow solid. m/z 286.0/288.0 (M+H)⁺ (ES+).

Step 3: 2-(2-(2,6-Dichloropyridin-4-yl)-5-fluorobenzoyl)-N-methylhydrazine-1-carbothioa mide (AAZ-4). Into a 100-mL round-bottom flask purged and maintained with an inert atmosphere of nitrogen, was placed the product from step 2 above (AAZ-3) (1.60 g, 1 Eq, 5.59 mmol) and 1-amino-3-methylthiourea (A-2) (710 mg, 1.2 Eq, 6.71 mmol) in DMF (50 mL) at rt. Then T3P (7.12 g, 4 Eq, 22.4 mmol) and DIPEA (2.17 g, 3 Eq, 16.8 mmol) were added at rt. The resulting solution was stirred for overnight at 60° C. The mixture was cooled to rt, purified by reverse flash column chromatography with the following conditions: Column, C18; mobile phase, Water (0.1% FA) and MeCN (23% MeCN up to 46% in 15 min); Detector, UV 254/220 nm. The product-containing fractions were combined and concentrated in vacuo. This resulted in the sub-title compound (AAZ-4) (810 g, 2.18 mmol, 39%) as brown yellow solid. m/z 373.0/375.0 (M+H)⁺ (ES+).

Step 4: 5-(2-(2,6-Dichloropyridin-4-yl)-5-fluorophenyl)-4-methyl-4H-1,2,4-triazole-3-thiol (AAZ-5). Into a 100-mL round-bottom flask, was placed the product from step 3 above (AAZ-4) (800 mg, 1 Eq, 2.14 mmol) in sat. NaHCO₃ solution (20 mL, 1 molar) at rt at rt. The resulting solution was stirred for overnight at 60° C. The mixture was cooled to rt, diluted with water and extracted with EtOAc (3×80 mL). The combined organic layers were washed with brine (2×80 mL), dried (Na₂SO₄). After filtration, the filtrate was concentrated in vacuo. This resulted in the sub-title compound (AAZ-5) (700 mg, 1.97 mmol, crude) as a brown solid. m/z 355.0/357.0 (M+H)⁺ (ES+).

Step 5: 2,6-Dichloro-4-(4-fluoro-2-(4-methyl-4H-1,2,4-triazol-3-yl)phenyl)pyridine (AAZ-6). Into a 100-mL round-bottom flask purged and maintained with an inert atmosphere of nitrogen, was placed the product from step 4 above (AAZ-5) (700 mg, 1 Eq, 1.97 mmol) in DCM (20 mL), then hydrogen peroxide (335 mg, 30% Wt, 5 Eq, 9.90 mmol) and AcOH (237 mg, 2 Eq, 3.94 mmol) were added at rt. The resulting solution was stirred for 3 h at rt. The resulting mixture was diluted with water and extracted with DCM (3×80 mL). The combined organic layers were washed with brine (2×80 mL), dried (Na₂SO₄). After filtration, the filtrate was concentrated in vacuo. This resulted in the sub-title compound (AAZ-6) (650 mg, 2.02 mmol, crude) as brown solid. m/z 323.0/325.0 (M+H)⁺ (ES+).

Step 6: 2-Chloro-6-cyclopropyl-4-(4-fluoro-2-(4-methyl-4H-1,2,4-triazol-3-yl)phenyl) pyridine (AAZ-7). Into a 50-mL round-bottom flask purged and maintained with an inert atmosphere of nitrogen, was placed the product from step 5 (AAZ-6) (650 mg, 1 Eq, 2.01 mmol), cyclopropylboronic acid (AAP-1) (207 mg, 1.2 Eq, 2.41 mmol) and potassium phosphate (1.28 g, 3 Eq, 6.03 mmol) in toluene (15 mL) at rt under nitrogen atmosphere. Then Pd(OAc)₂ (59 mg, 0.13 Eq, 261 μmol) and tricyclohexylphosphane (73 mg, 0.13 Eq, 261 μmol) were added at rt under nitrogen atmosphere. The resulting solution was stirred for 3 h at 110° C. under nitrogen atmosphere. The mixture was cooled to rt and concentrated in vacuo. The residue was purified by Prep-TLC with DCM/MeOH (10/1). This resulted in the sub-title compound (AAZ-7) (310 mg, 945 μmol, 47%) as a brown yellow solid. m/z 329.1/331.1 (M+H)⁺ (ES+).

Step 7: 6-Cyclopropyl-4-(4-fluoro-2-(4-methyl-4H-1,2,4-triazol-3-yl)phenyl)picolinic acid (AAZ-8). Into a 50-mL round-bottom flask purged and maintained with an inert atmosphere of nitrogen, was placed the product from step 6 above (AAZ-7) (300 mg, 1 Eq, 912 μmol), oxalic acid (99 mg, 1.2 Eq, 1.09 mmol), acetic anhydride (140 mg, 1.5 Eq, 1.37 mmol) and DIPEA (177 mg, 1.5 Eq, 1.37 mmol) in DMF (10 mL) at rt under nitrogen atmosphere. Then Pd(OAc)₂ (207 mg, 0.1 Eq, 791 μmol) and XantPhos (106 mg, 0.2 Eq, 182 mmol) were added at rt under nitrogen atmosphere. The resulting solution was stirred for 3 h at 100° C. under nitrogen atmosphere. The mixture was cooled to rt. The crude product was purified by reverse flash column chromatography with the following conditions: Column, C18; mobile phase, Water (0.1% FA) and MeCN (15% MeCN up to 30% in 10 min); Detector, UV 254/220 nm. The product-containing fractions were combined and concentrated in vacuo. This resulted in the sub-title compound (AAZ-8) (120 mg, 355 μmol, 39%) as a brown yellow solid. m/z 339.1 (M+H)⁺ (ES+).

Step 8: (S)—N-(2-Amino-5-((3-methylpiperidin-1-yl)methyl)-3-(trifluoromethyl)phenyl)-6-cyclopropyl-4-(4-fluoro-2-(4-methyl-4H-1,2,4-triazol-3-yl)phenyl)picolinamide (AAZ-9). Into a 25-mL round-bottom flask purged and maintained with an inert atmosphere of nitrogen, was placed the product from step 7 above (AAZ-8) (35 mg, 1 Eq, 103 μmol) and intermediate (AAX-2) (36 mg, 1.2 Eq, 124 μmol) in DMF (5 mL) at rt under nitrogen atmosphere. Then HATU (39 mg, 1 Eq, 103 μmol) and DIPEA (40 mg, 3 Eq, 309 μmol) were added at rt under nitrogen atmosphere. The resulting solution was stirred for 3 h at rt under nitrogen atmosphere. The crude product was purified by reverse flash column chromatography with the following conditions: Column, C18; mobile phase, Water (0.1% NH₄HCO₃) and MeCN (60% MeCN up to 85% in 15 min); Detector, UV 254/220 nm. The product-containing fractions were combined and concentrated in vacuo. This resulted in the sub-title compound (AAZ-9) (35 mg, 58 μmol, 56%) as a yellow solid. m/z 608.3 (M+H)+ (ES+).

Step 9: (S)-2-(6-Cyclopropyl-4-(4-fluoro-2-(4-methyl-4H-1,2,4-triazol-3-yl)phenyl)pyridin-2-yl)-5-((3-methylpiperidin-1-yl)methyl)-7-(trifluoromethyl)-1H-benzo[d]imidazole (AAZ-10). Into a 25-mL round-bottom flask, was placed the product from step 8 above (AAZ-9) (35 mg, 1 Eq, 58 μmol) in AcOH (3 mL). The resulting solution was stirred for overnight at 80° C. under nitrogen atmosphere. The mixture was cooled to rt and concentrated in vacuo. The crude product was purified by Prep-HPLC with the following conditions: Column: XBridge Prep OBD C18 Column, 30*150 mm, 5 μm; Mobile Phase A: Water (0.1% NH₄HCO₃), Mobile Phase B: MeCN; Flow rate: 60 mL/min; Gradient: 55% B to 70% B in 9 min; Wave Length: 254/220 nm; RT: 8.14. This resulted in the title compound (AAZ-10) (17.7 mg, 30 μmol, 52%) as a white solid. m/z 590.3 (M+H)⁺ (ES+). ¹H NMR (400 MHz, Methanol-d4) δ 8.47 (s, 1H), 8.19 (s, 1H), 7.92-7.82 (m, 2H), 7.64-7.55 (m, 2H), 7.51 (dd, J=8.7, 2.7 Hz, 1H), 6.92 (d, J=1.6 Hz, 1H), 3.73 (s, 2H), 3.36 (s, 3H), 2.97-2.86 (m, 2H), 2.12-1.98 (m, 2H), 1.80-1.58 (m, 5H), 1.16-1.02 (m, 4H), 0.98-0.87 (m, 4H).

### Example 106: Synthesis of 5-((5-Azaspiro[2.4]heptan-5-yl)methyl)-2-(6-cyclopropyl-4-(4-fluoro-2-(4-methyl-4H-1,2,4-triazol-3-yl)phenyl)pyridin-2-yl)-7-(trifluoromethyl)-1H-benzo[d]imidazole (ABA-2)

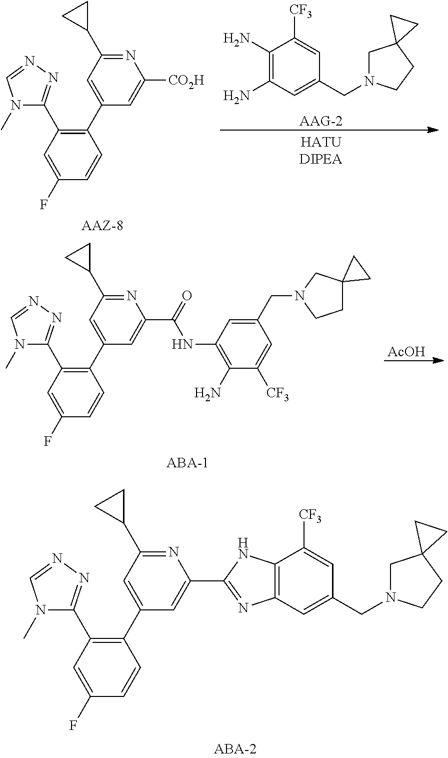

Step 1: N-(5-((5-Azaspiro[2.4]heptan-5-yl)methyl)-2-amino-3-(trifluoromethyl)phenyl)-6-cyclopropyl-4-(4-fluoro-2-(4-methyl-4H-1,2,4-triazol-3-yl)phenyl)picolinamide (ABA-1). Into a 25-mL round-bottom flask purged and maintained with an inert atmosphere of nitrogen, was placed intermediate (AAZ-8) (120 mg, 1 Eq, 355 μmol) and intermediate (AAG-2) (121 mg, 1.2 Eq, 426 μmol) in DMF (5 mL) at rt, then HATU (135 mg, 1 Eq, 355 μmol) and DIPEA (138 mg, 3 Eq, 1.07 mmol) were added at rt. The resulting solution was stirred for 3 h at rt. The crude product was purified by reverse flash column chromatography with the following conditions: Column, C18; mobile phase, Water (0.1% NH₄HCO₃) and MeCN (60% MeCN up to 85% in 10 min); Detector, UV 254/220 nm. The product-containing fractions were combined and concentrated in vacuo. This resulted in the sub-title compound (ABA-1) (80 mg, 132 μmol, 37%) as a brown yellow solid. m/z 606.3 (M+H)⁺ (ES+).

Step 2: 5-((5-Azaspiro[2.4]heptan-5-yl)methyl)-2-(6-cyclopropyl-4-(4-fluoro-2-(4-methyl-4H-1,2,4-triazol-3-yl)phenyl)pyridin-2-yl)-7-(trifluoromethyl)-1H-benzo[d]imidazole (ABA-2). Into a 25-mL round-bottom flask, was placed the product from step 1 above (ABA-1) (80 mg, 1 Eq, 132 μmol) in AcOH (5 mL). The resulting solution was stirred overnight at 100° C. The mixture was cooled to rt and concentrated in vacuo. The crude product was purified by Prep-HPLC with the following conditions: Column: Sunfire prep C18 column, 30*150 mm, 5 μm; Mobile Phase A: Water (0.05% TFA), Mobile Phase B: MeCN; Flow rate: 60 mL/min; Gradient: 14% B to 35% B in 7 min; Detector, UV 210/254 nm; RT: 6.72. This resulted in the title compound (ABA-2) (6.5 mg, 11 μmol, 8.4%) as a white solid. m/z 588.2 (M+H)⁺ (ES+). ¹H NMR (400 MHz, Methanol-d4) δ8.47 (s, 1H), 8.16 (s, 1H), 7.95 (s, 1H), 7.92-7.83 (m, 1H), 7.71 (s, 1H), 7.64-7.48 (m, 2H), 6.97 (d, J=1.6 Hz, 1H), 4.25 (s, 2H), 3.39 (s, 3H), 3.22 (s, 2H), 2.95 (s, 2H), 2.11 (s, 1H), 2.02 (t, J=7.2 Hz, 2H), 1.14-1.00 (m, 4H), 0.73-0.67 (m, 4H).

### Example 107: Synthesis of 2-(3-(3-((4-Methyl-4H-1,2,4-triazol-3-yl)methyl)oxetan-3-yl) phenyl)-7-(trifluoromethyl)-1H-benzo[d]imidazole (ABB-2)

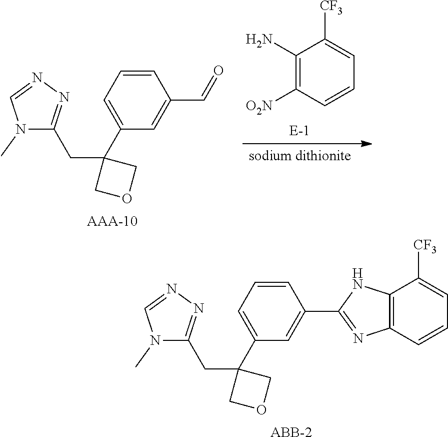

To a stirred solution of intermediate (AAA-10) (100 mg, 1 Eq, 389 μmol) and 2-nitro-6-(trifluoromethyl) aniline (E-1) (120 mg, 1.5 Eq, 584 μmol) in MeOH (5 mL) was added sodium dithionite (135 mg, 2 Eq, 778 μmol) at rt. The resulting mixture was stirred for overnight at 80° C. under nitrogen atmosphere. The mixture was cooled to rt and concentrated in vacuo. The crude product was purified by reverse flash column chromatography with the following conditions: Column, C18; mobile phase, Water (0.1% FA) and MeCN (20% MeCN up to 50% in 10 min); Detector, UV 254/220 nm. The product-containing fractions were combined and concentrated in vacuo. The crude product was purified by Prep-HPLC with the following conditions: Column: Atlantis HILIC OBD Column, 19×150 mm 5 um; Mobile Phase A: Water (0.1% FA), Mobile Phase B: MeCN; Flow rate: 60 mL/min; Gradient: 25 B to 40 B in 7 min; Detector, UV 254/210 nm; RT: 6.63. The product-containing fractions were combined and evaporated partially in vacuo and lyophilized overnight to afford the title compound (ABB-2) (19.4 mg, 47 μmol, 12%) as a white solid. m/z 414.3 (M+H)⁺ (ES+). ¹H NMR (300 MHz, Methanol-d4) δ 8.17 (s, 1H), 8.09 (d, J=7.8 Hz, 1H), 7.81 (s, 2H), 7.58 (d, J=7.6 Hz, 1H), 7.50 (t, J=7.8 Hz, 1H), 7.41 (t, J=7.9 Hz, 1H), 7.08-6.98 (m, 1H), 5.19-5.09 (m, 4H), 3.70 (s, 2H), 2.92 (s, 3H).

### Example 108: Synthesis of 2-(3-(3-((4-Methyl-4H-1,2,4-triazol-3-yl)methyl)oxetan-3-yl) phenyl)-5-(trifluoromethyl)-[1,2,4]triazolo[1,5-a]pyridine (ABC-4)

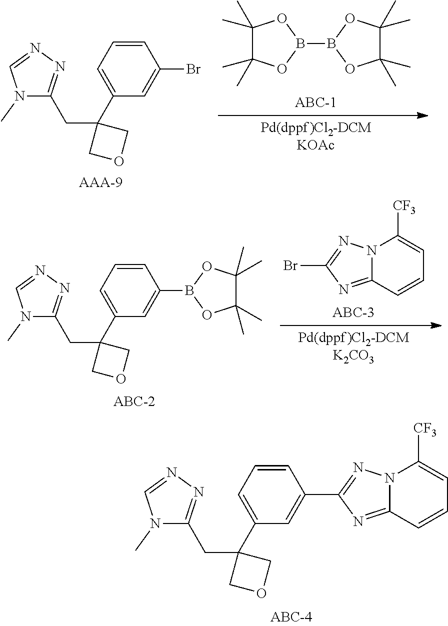

Step 1: 4-Methyl-3-((3-(3-(4,4,5,5-tetramethyl-1,3,2-dioxaborolan-2-yl)phenyl)oxetan-3-yl) methyl)-4H-1,2,4-triazole (ABC-2). To a stirred solution of intermediate (AAA-9) (250 mg, 1 Eq, 811 μmol), bis(pinacolato)diboron (ABC-1) (618 mg, 3 Eq, 2.43 mmol) and KOAc (239 mg, 3 Eq, 2.43 mmol) in dioxane (5 mL) was added Pd(dppf)Cl₂-DCM (59 mg, 0.1 Eq, 81 μmol) at rt under nitrogen atmosphere. The resulting mixture was stirred for 2 h at 100° C. under nitrogen atmosphere. The mixture was cooled to rt and concentrated in vacuo. The crude product was purified by reverse flash column chromatography with the following conditions: Column, C18; mobile phase, Water (0.1% NH₄HCO₃) and MeCN (10% MeCN up to 70% in 15 min); Detector, UV 254/220 nm. The product-containing fractions were combined and concentrated in vacuo. This resulted in the sub-title compound (ABC-2) (220 mg, 929 μmol, 76%) as a white solid. m/z 356.2 (M+H)⁺ (ES+).

Step 2: 2-(3-(3-((4-Methyl-4H-1,2,4-triazol-3-yl)methyl)oxetan-3-yl)phenyl)-5-(trifluoromethyl)-[1,2,4]triazolo[1,5-a]pyridine (ABC-4). To a stirred solution of 2-bromo-5-(trifluoromethyl)-[1,2,4]triazolo[1,5-a]pyridine (ABC-3) (53 mg, 1 Eq, 199 μmol), the product from step 1 above (ABC-2) (85 mg, 1.2 Eq, 239 μmol) and K₂CO₃ (83 mg, 3 Eq, 598 μmol) in 1,4-dioxane (4 mL) and water (1 mL) was added Pd(dppf)Cl₂-DCM (16 mg, 0.1 Eq, 20 μmol) at rt under nitrogen atmosphere. The resulting mixture was stirred for 1 h at 100° C. under nitrogen atmosphere. The mixture was allowed to cool down to rt. The resulting mixture was diluted with water (30 mL). The resulting mixture was extracted with EtOAc (3×30 mL). The combined organic layers were washed with brine (1×30 mL), dried (Na₂SO₄) and concentrated in vacuo. The crude product was purified by Prep-HPLC with the following conditions (Column: Column: YMC-Actus Triart C18, 30*250.5 um; Mobile Phase A: Water (0.1% NH₄HCO₃+0.1% NH₃·H₂O), Mobile Phase B: MeCN; Flow rate: 60 mL/min; Gradient: 20 B to 50 B in 10 min; Detector, UV 210/254 nm; RT: 8.97) to afford the title compound (ABC-4) (34.5 mg, 83 μmol, 42%) as a white solid. m/z 415.1 (M+H)⁺ (ES+). ¹H NMR (400 MHz, Methanol-d4) δ 8.25-8.15 (m, 2H), 8.07 (d, J=9.0 Hz, 1H), 7.88-7.78 (m, 2H), 7.72 (d, J=7.3 Hz, 1H), 7.50 (t, J=7.8 Hz, 1H), 7.07-7.02 (m, 1H), 5.19-5.08 (m, 4H), 3.71 (s, 2H), 2.88 (s, 3H).

### Example 109: Synthesis of 2-{3-[(2R)-1-(4-Methyl-1,2,4-triazol-3-yl)propan-2-yl]phenyl}-4-(trifluoromethyl)-3H-1,3-benzodiazole (ABD-9)

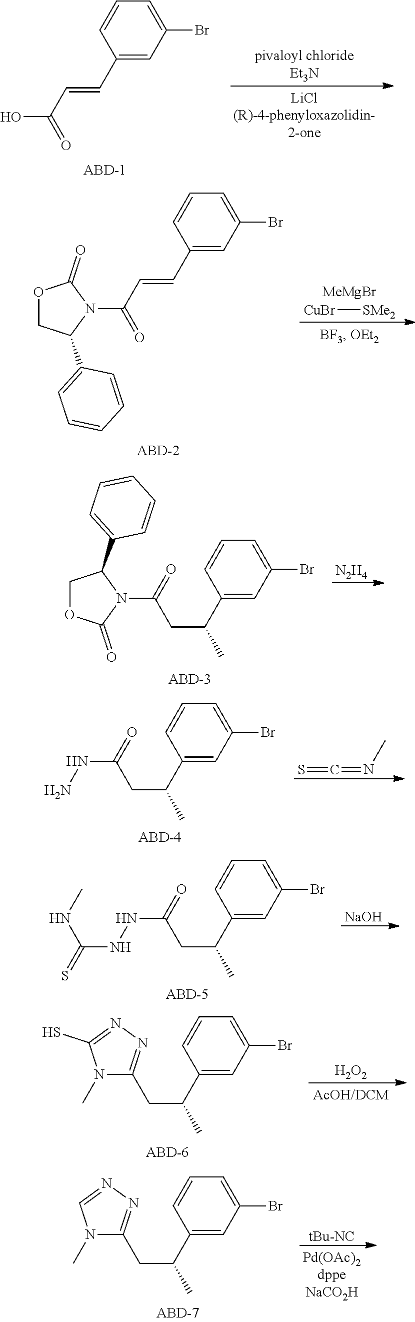

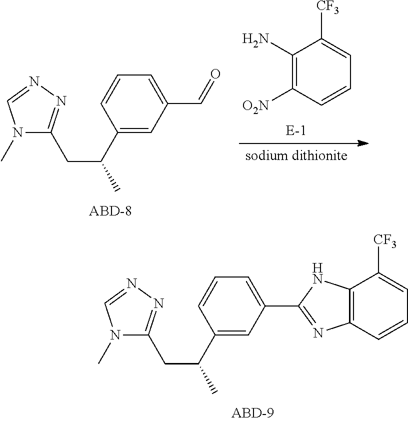 [SMILES: C[C@H](Cc1nncn1C)c1cccc(C=O)c1 ; Nc1c([N+](=O)[O-])cccc1C(F)(F)F ; C[C@H](Cc1nncn1C)c1cccc(-c2nc3cccc(C(F)(F)F)c3[nH]2)c1]

Step 1: (4R)-3-[(2E)-3-(3-Bromophenyl)prop-2-enoyl]-4-phenyl-1,3-oxazolidin-2-one (ABD-2). Into a 500 mL round-bottom flask were added m-bromocinnamic acid (ABD-1) (4.54 g, 1 Eq, 20.0 mmol) and Et₃N (4.45 g, 2.2 Eq, 44.0 mmol) in THF (70 mL) at rt. To the above mixture was added pivaloyl chloride (2.41 g, 1 Eq, 20.0 mmol) dropwise over 3 min at 0° C. The resulting mixture was stirred for additional 1 h at 0° C. under nitrogen atmosphere. To the above mixture was added LiCl (850 mg, 1 Eq, 20.0 mmol) and (4S)-4-phenyl-1,3-oxazolidin-2-one (3.26 g, 1 Eq, 20.0 mmol) at 0° C. The resulting mixture was stirred overnight at rt under nitrogen atmosphere. The reaction was quenched with sat. NH₄Cl solution at 0° C. The resulting mixture was diluted with water and extracted with EtOAc (3×300 mL). The combined organic layers were washed with brine (2×300 mL), dried (Na₂SO₄). After filtration, the filtrate was concentrated in vacuo. The residue was applied on a silica gel column chromatography with petroleum ether/EtOAc (3/1). This resulted in the sub-title compound (ABD-2) (5.2 g, 14.0 mmol, 70%) as a yellow solid. m/z 372.0/374.0 (M+H)⁺ (ES+).

Step 2: (4R)-3-[(3R)-3-(3-Bromophenyl)butanoyl]-4-phenyl-1,3-oxazolidin-2-one (ABD-3). Into a mL 3-necked round-bottom flask were added bromodimethylsulfanylidenecopper (4.52 g, 3 Eq, 22.0 mmol) and bromo(methyl)magnesium (14.7 mL, 6 Eq, 44.0 mmol) in THF (100 mL) at −40° C. under nitrogen atmosphere. To the above mixture was added BF₃·Et₂O (3.12 g, 3 Eq, 22.0 mmol) in THF (30 mL) dropwise over 40 min at −40° C. under nitrogen atmosphere. The resulting mixture was stirred for additional 40 min at −25° C. To the above mixture was added the product from step 1 above (ABD-2) (2.73 g, 1 Eq, 7.33 mmol) in THF (30 mL) dropwise at −40° C. The resulting mixture was stirred for additional 2 h at -20° C. under nitrogen atmosphere. The reaction was quenched with sat. NH₄Cl solution at −40° C. The resulting mixture was diluted with water and extracted with EtOAc (3×200 mL). The combined organic layers were washed with brine (2×200 mL), dried (Na₂SO₄). After filtration, the filtrate was concentrated in vacuo. The residue was applied on a silica gel column chromatography with petroleum ether/EtOAc (3/1). This resulted in the sub-title compound (ABD-3) (1.9 g, 4.90 mmol, 67%) as a white solid. m/z 388.0/390.0 (M+H)⁺ (ES+).

Step 3: (3R)-3-(3-Bromophenyl)butanehydrazide (ABD-4). Into a 250 mL round-bottom flask were added the product from step 2 above (ABD-3) (1.90 g, 1 Eq, 4.89 mmol) in THF (20 mL) at rt. To the above mixture was added hydrazine (0.5 mL, 2 Eq, 9.78 mmol) dropwise over 3 min at rt. The resulting mixture was stirred overnight at rt. The resulting mixture was concentrated in vacuo to afford the sub-title compound (ABD-4) (1.2 g, 4.67 mmol, 95%) as colorless oil. m/z 257.0/259.0 (M+H)⁺ (ES+).

Step 4: (3R)-3-(3-Bromophenyl)-N-[(methylcarbamothioyl)amino]butanamide (ABD-5). Into a 250 mL round-bottom flask were added the product from step 3 above (ABD-4) (1.20 g, 1 Eq, 4.67 mmol) in THF (15 mL) at rt. To the above mixture was added methyl isothiocyanate (410 mg, 1.2 Eq, 5.60 mmol) over 3 min at rt. The resulting mixture was stirred overnight at rt. The crude resulting mixture was used in the next step directly without further purification. m/z 330.0/332.0 (M+H)⁺ (ES+).

Step 5: 5-[(2R)-2-(3-Bromophenyl)propyl]-4-methyl-1,2,4-triazole-3-thiol (ABD-6). Into a 250 mL round-bottom flask were added the product from step 4 above (ABD-5) (1.60 g, 1 Eq, 4.85 mmol) in THE (15 mL) at rt. To the above mixture was added NaOH (390 mg, 2 Eq, 9.69 mmol) dropwise over 3 min at rt. The resulting mixture was stirred for additional 3 h at rt. The resulting mixture was diluted with water. The residue was acidified to pH 3 with HCl (aq.) and extracted with EtOAc (3×100 mL). The combined organic layers were washed with brine (2×100 mL), dried (Na₂SO₄). After filtration, the filtrate was concentrated in vacuo. The residue was applied on a silica gel column chromatography with petroleum ether/EtOAc (4/1). This resulted in the sub-title compound (ABD-6) (1.04 g, 3.34 mmol, 69%) as a white solid. m/z 312.0/314.0 (M+H)⁺ (ES+).

Step 6: 3-[(2R)-2-(3-Bromophenyl)propyl]-4-methyl-1,2,4-triazole (ABD-7). Into a 250 mL round-bottom flask were added the product from step 5 above (ABD-6) (1.00 g, 1 Eq, 3.20 mmol) and AcOH (380 g, 2 Eq, 6.41 mmol) in DCM (15 mL) at rt. To the above mixture was added hydrogen peroxide (0.30 mL, 30% Wt, 4 Eq, 12.8 mmol) dropwise over 3 min at 0° C. The resulting mixture was stirred for additional 3 h at rt. The resulting mixture was diluted with water and extracted with DCM (3×100 mL). The combined organic layers were washed with brine (2×100 mL), dried (Na₂SO₄). After filtration, the filtrate was concentrated in vacuo. The crude product was purified by reverse flash column chromatography with the following conditions: Column, C18; mobile phase, Water (0.1% NH₄HCO₃) and MeCN (36% MeCN up to 48% in 10 min); Detector, UV 254/220 nm. The product-containing fractions were combined and concentrated in vacuo. This resulted in the sub-title compound (ABD-7) (780 mg, 2.80 mmol, 87%) as yellow oil. m/z 280.0/282.0 (M+H)⁺ (ES+).

Step 7: 3-[(2R)-1-(4-Methyl-1,2,4-triazol-3-yl)propan-2-yl]benzaldehyde (ABD-8). Into a 40 mL sealed tube were added the product from step 6 above (ABD-7) (300 mg, 1 Eq, 1.07 mmol), tert-butyl isocyanide (107 mg, 1.2 Eq, 1.29 mmol) and sodium formate (146 mg, 2 Eq, 2.14 mmol) in DMSO (6 mL) at rt. To the above mixture was added diphos (85 mg, 0.2 Eq, 214 μmol) and Pd(OAc)₂ (24 mg, 0.1 Eq, 107 μmol) over 3 min at rt under nitrogen atmosphere. The resulting mixture was stirred for additional 2 h at 120° C. The mixture was allowed to cool down to rt. The crude product was purified by reverse flash column chromatography with the following conditions: Column, C18; mobile phase, Water (0.1% NH₄HCO₃) and MeCN (36% MeCN up to 48% in 10 min); Detector, UV 254/220 nm. The product-containing fractions were combined and concentrated in vacuo. This resulted in the sub-title compound (ABD-8) (100 mg, 436 μmol, 41%) as yellow oil. m/z 230.1 (M+H)⁺ (ES+).

Step 8: 2-{3-[(2R)-1-(4-Methyl-1,2,4-triazol-3-yl)propan-2-yl]phenyl}-4-(trifluoromethyl)-3H-1,3-benzodiazole (ABD-9). Into an 8 mL sealed tube were added the product from step 7 above (ABD-8) (50 mg, 1 Eq, 218 μmol) and intermediate (E-1) (90 mg, 2 Eq, 436 μmol) in EtOH (2 mL) at rt. To the above mixture was added sodium hydrosulfite (190 mg, 5 Eq, 1.09 mmol) in water (1 mL) dropwise over 10 min at rt. The resulting mixture was stirred for additional 3 h at 70° C. The mixture was cooled to rt and concentrated in vacuo. The crude product was purified by Prep-HPLC with the following conditions: Column: SunFire Prep C18 OBD Column, 19×150 mm, 5 μm; Mobile Phase A: Water (0.1% NH₄HCO₃+0.1% NH₃·H₂O), Mobile Phase B: MeCN; Flow rate: 60 mL/min; Gradient: 30 B to 60 B in 7 min; Detector, UV 254/210 nm; RT: 6.08 to afford the title compound (ABD-9) (22.6 mg, 27%) as a white solid. m/z 386.3 (M+H)⁺ (ES+). ¹H NMR (400 MHz, Methanol-d4) δ 8.29 (s, 1H), 8.14-8.10 (m, 1H), 8.05-8.01 (m, 1H), 7.87 (d, J=8.1 Hz, 1H), 7.60-7.57 (m, 1H), 7.53-7.37 (m, 3H), 3.52 (s, 3H), 3.49-3.41 (m, 1H), 3.26-3.14 (m, 2H), 1.50 (d, J=6.9 Hz, 3H).

### Example 110: Synthesis of 5-Bromo-2-(3-(3-((4-methyl-4H-1,2,4-triazol-3-yl)methyl) oxetan-3-yl) phenyl)-7-(trifluoromethyl)-1H-benzo[d]imidazole (ABE-2)

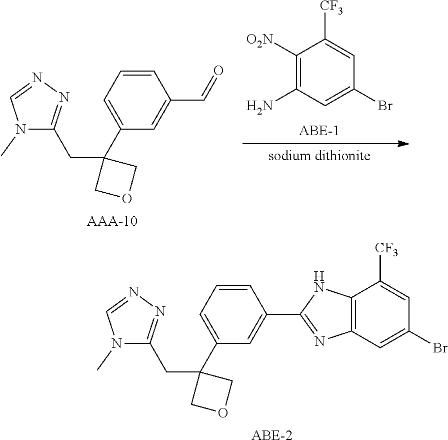

Into an 8 mL sealed tube were added intermediate (AAA-10) (58 mg, 1 Eq, 225 μmol) and 5-bromo-2-nitro-3-(trifluoromethyl) aniline (ABE-1) (129 mg, 2 Eq, 450 μmol) in EtOH (3 mL) at rt. To the above mixture was added sodium dithionite (196 mg, 5 Eq, 1.13 mmol) in water (2 mL) dropwise over 10 min at 0° C. The resulting mixture was stirred for additional 3 h at 70° C. The mixture was cooled to rt and concentrated in vacuo. The crude product was purified by Prep-HPLC with the following conditions: Column: SunFire Prep C18 OBD Column, 19×150 mm, 5 μm; Mobile Phase A: Water (0.1% NH₄HCO₃+0.1% NH₃·H₂O), Mobile Phase B: MeCN; Flow rate: 60 mL/min; Gradient: 30 B to 60 B in 7 min; Detector, UV 254/210 nm; RT: 6.08 to afford the title compound (ABE-2) (7.4 mg, 6.6%) as a white solid. m/z 492.0/494.0 (M+H)⁺ (ES+) ¹H NMR (400 MHz, Methanol-d4) δ 8.18 (s, 1H), 8.12-8.01 (m, 1H), 8.00 (s, 1H), 7.82 (t, J=1.8 Hz, 1H), 7.69 (d, J=1.8 Hz, 1H), 7.52 (t, J=7.8 Hz, 1H), 7.09-7.04 (m, 1H), 5.15 (s, 4H), 3.71 (s, 2H), 2.94 (s, 3H).

### Example 111: Synthesis of 2-(3-(3-((4-Methyl-4H-1,2,4-triazol-3-yl)methyl)oxetan-3-yl) phenyl)-4-(trifluoromethyl)benzo[d]thiazole (ABF-4)

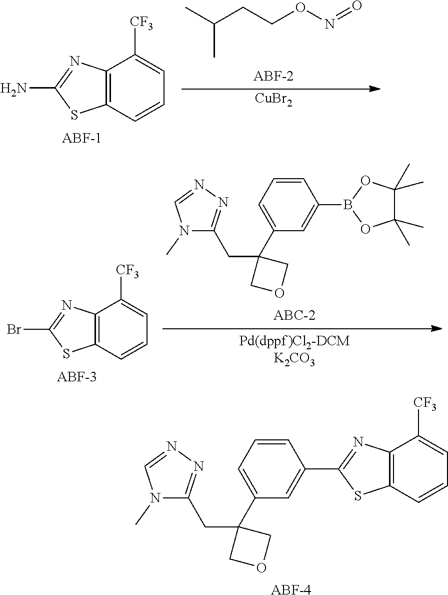

Step 1: 2-(3-[3-[(4-Methyl-1,2,4-triazol-3-yl)methyl]oxetan-3-yl]phenyl)-1,3-benzothiazole (ABF-3). To a stirred solution of 4-(trifluoromethyl)-1,3-benzothiazol-2-amine (ABF-1) (218 mg, 1 Eq, 999 mmol) and isoamyl nitrite (176 mg, 1.5 Eq, 1.50 mmol) in MeCN (10 mL) was added CuBr₂ (335 mg, 1.5 Eq, 1.50 mmol) dropwise at 0° C. The resulting mixture was stirred overnight at rt. The resulting mixture was concentrated in vacuo. The residue was purified by Prep-TLC hexane/EtOAc (40/1) to afford the sub-title compound (ABF-3) (260 mg, 926 μmol, 92%) as an off-white solid. m/z 281.9/283.9 (M+H)⁺ (ES+).

Step 2: 2-(3-[3-[(4-Methyl-1,2,4-triazol-3-yl)methyl]oxetan-3-yl]phenyl)-4-(trifluoromethyl)-1,3-benzothiazole (ABF-4). To a stirred solution of intermediate (ABC-2) (50 mg, 1 Eq, 141 μmol), the product from step 1 above (ABF-3) (40 mg, 1 Eq, 141 μmol) and K₂CO₃ (58 mg, 3 Eq, 423 μmol) in 1,4-dioxane (4 mL) and water (1 mL) was added Pd(dppf)Cl₂-DCM (11 mg, 0.1 Eq, 14 μmol) at rt under nitrogen atmosphere. The final reaction mixture was irradiated with microwave radiation for 2 h at 100° C. The mixture was allowed to cool down to rt. The crude product was purified by reverse flash column chromatography with the following conditions: Column, C18; mobile phase, Water (0.1% FA) and MeCN (20% MeCN up to 60% in 20 min); Detector, UV 254/220 nm. The crude product was purified by Prep-HPLC with the following conditions (Column: SunFire Prep C18 OBD Column, 19×150 mm 5 μm 10 nm; Mobile Phase A: Water (0.1% FA), Mobile Phase B: MeCN; Flow rate: 60 mL/min; Gradient: 30 B to 50 B in 7 min; 254/210 nm; RT: 10.38) to afford the title compound (ABF-4) (24.8 mg, 58 μmol, 41%) as a white solid. m/z 431.3 (M+H)⁺ (ES+). ¹H NMR (400 MHz, CD30D) δ 8.99 (s, 1H), 8.32 (d, J=8.1 Hz, 1H), 8.13-8.10 (m, 1H), 8.09 (d, J=7.8 Hz, 1H), 7.72-7.69 (m, 1H), 7.64-7.54 (m, 2H), 7.34-7.29 (m, 1H), 5.18-5.08 (m, 4H), 3.86 (s, 2H), 3.22 (s, 3H).

### Example 112: Synthesis of 7-Cyclopropyl-2-(3-(3-((4-methyl-4H-1,2,4-triazol-3-yl) methyl)oxetan-3-yl)phenyl)-1H-benzo[d]imidazole (ABG-3)

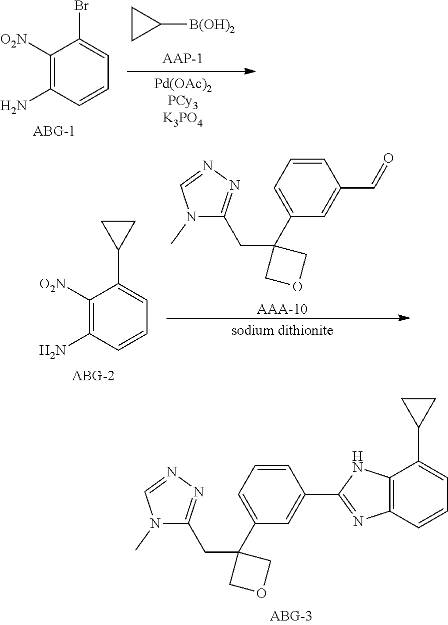

Step 1: 3-Cyclopropyl-2-nitroaniline (ABG-2). To a solution of 3-bromo-2-nitroaniline (ABG-1) (1.00 g, 1 Eq, 4.61 mmol) and cyclopropylboronic acid (AAP-1) (594 mg, 1.5 Eq, 6.91 mmol) in toluene (20 mL) and water (2 mL) were added Pd(AcO)₂ (103 mg, 0.1 Eq, 461 μmol), PCy₃ (258 mg, 922 μmol, 0.2 Eq) and potassium phosphate (2.93 g, 3 Eq, 13.8 mmol). After stirring for overnight at 100° C. under a nitrogen atmosphere, the mixture was allowed to cool down to rt and concentrated. The residue was purified by silica gel column chromatography with petroleum ether/EtOAc (5/1) to afford the sub-title compound (ABG-2) (500 mg, 2.81 mmol, 61%) as a brown solid. m/z 179.1 (M+H)⁺ (ES+).

Step 2: 3-[3-[(4-Methyl-1,2,4-triazol-3-yl)methyl]oxetan-3-yl]benzaldehyde (ABG-3). To a stirred solution of intermediate (AAA-10) (51 mg, 1 Eq, 200 μmol) and the product from step 1 above (ABG-2) (36 mg, 1 Eq, 200 mmol) in EtOH (4 mL) were added sodium hydrosulfite (1 mL, 1 molar) at rt under nitrogen atmosphere. The resulting mixture was stirred for 4 h at 80° C. under nitrogen atmosphere. The mixture was cooled to rt and concentrated in vacuo. The crude product was purified by reverse flash column chromatography with the following conditions: Column, C18; mobile phase, Water (0.1% NH₄HCO₃) and MeCN (10% MeCN up to 50% in 10 min); Detector, UV 254/220 nm. The crude product was purified by Prep-HPLC with the following conditions (Column: XBridge Prep C18 OBD Column, 19*150 mm 5 um; Mobile Phase A: Water (0.1% NH₄HCO₃+0.1% NH₃·H₂O), Mobile Phase B: MeCN; Flow rate: 60 mL/min; Gradient: 20 B to 35 B in 10 min; 254/210 nm;) to afford the title compound (ABG-3) (37.6 mg, 98 μmol, 49%) as a white solid. m/z 386.1 (M+H)⁺ (ES+). ¹H NMR (300 MHz, CD30D) δ 8.18 (s, 1H), 8.08 (d, J=7.8 Hz, 1H), 7.79-7.75 (m, 1H), 7.52-7.34 (m, 2H), 7.19-7.11 (m, 1H), 7.01 (d, J=7.8 Hz, 1H), 6.81 (d, J=7.5 Hz, 1H), 5.17-5.09 (m, 4H), 3.70 (s, 2H), 2.92 (s, 3H), 2.52 (s, 1H), 1.14-1.04 (m, 2H), 0.88-0.82 (m, 2H).

### Example 113: Synthesis of (2-(3-(3-((4-Methyl-4H-1,2,4-triazol-3-yl)methyl)oxetan-3-yl)phenyl)-7-(trifluoromethyl)-1H-benzo[d]imidazol-5-yl)(pyrrolidin-1-yl)methanone (ABH-4)

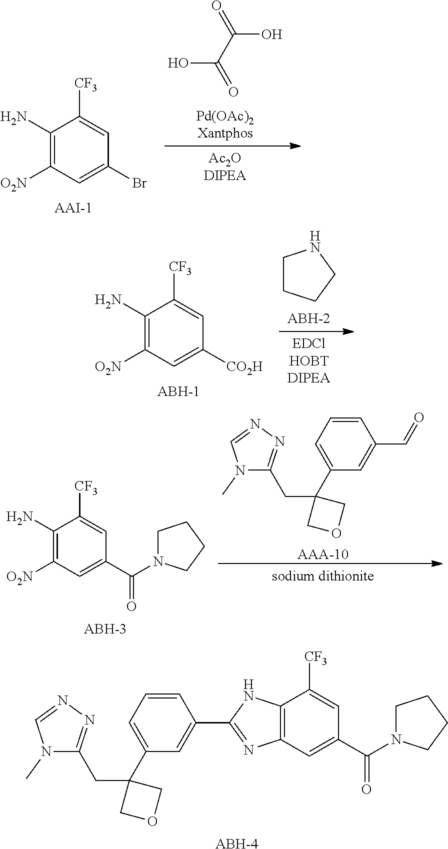

Step 1: 4-amino-3-nitro-5-(trifluoromethyl)benzoic acid (ABH-1). To a solution of 4-bromo-2-nitro-6-(trifluoromethyl)aniline (AAI-1) (710 mg, 1 Eq, 2.49 mmol) and oxalic acid dihydrate (471 mg, 1.5 Eq, 3.74 mmol) in DMF (10 mL) were added acetic anhydride (382 mg, 1.5 Eq, 3.74 mmol) and DIPEA (483 mg, 1.5 Eq, 3.74 mmol) dropwise, then Pd(AcO)₂ (56 mg, 0.1 Eq, 249 μmol) and XantPhos (288 mg, 0.2 Eq, 498 μmol) under nitrogen atmosphere. The resulting mixture was stirred for overnight at 100° C. under nitrogen atmosphere. The mixture was cooled to rt, diluted with water and extracted with EtOAc (3×80 mL). The combined organic layers were washed with brine (2×80 mL), dried (Na₂SO₄). After filtration, the filtrate was concentrated in vacuo. The residue was purified by reverse flash chromatography with the following conditions: (column, C18 silica gel; mobile phase, MeCN in water (0.1% FA), 10% to 80% gradient in 20 min; detector, UV 254/220 nm) to afford the sub-title compound (ABH-1) (200 mg, 800 μmol, 32%) as a brown solid. m/z 251.0 (M+H)⁺ (ES+)

Step 2: (4-Amino-3-nitro-5-(trifluoromethyl)phenyl)(pyrrolidin-1-yl)methanone (ABH-3). To a stirred mixture of the product from step 1 above (ABH-1) (200 mg, 1 Eq, 800 mmol) and HOBt (162 mg, 1.5 Eq, 1.20 mmol) in DMF (6 mL) was added pyrrolidine (ABH-2) (114 mg, 2 Eq, 1.60 mmol) and DIPEA (310 mg, 3 Eq, 2.40 mmol) at rt. Then to this was added EDCI (230 mg, 1.5 Eq, 1.20 mmol). The resulting mixture was stirred for overnight at rt. The residue was purified by reverse flash chromatography with the following conditions: (column, C18 silica gel; mobile phase, MeCN in water (0.1% NH₄HCO₃), 10% to 80% gradient in 20 min; detector, UV 254/220 nm) to afford the sub-title compound (ABH-3) (180 mg, 594 μmol, 74%) as a brown solid. m/z 304.1(M+H)⁺ (ES+).

Step 3: (2-(3-(3-((4-Methyl-4H-1,2,4-triazol-3-yl)methyl)oxetan-3-yl)phenyl)-7-(trifluoromethyl)-1H-benzo[d]imidazol-5-yl)(pyrrolidin-1-yl)methanone (ABH-4). To a stirred mixture of intermediate (AAA-10) (50 mg, 1 Eq, 194 μmol) and the product from step 2 above (ABH-3) (59 mg, 1 Eq, 194 μmol) in EtOH (2 mL) was added a solution of disodium hydrosulfite (338 mg, 10 Eq, 1.94 mmol) in water (2 mL) at rt under nitrogen atmosphere. The resulting mixture was stirred for overnight at 80° C. under nitrogen atmosphere. The mixture was cooled down to rt and concentrated in vacuo. The residue was purified by reverse flash chromatography with the following conditions: (column, C18 silica gel; mobile phase, MeCN in water (0.1% NH₄HCO₃), 10% to 80% gradient in 20 min; detector, UV 254/220 nm) and Prep-HPLC with the following conditions: (Column: YMC-Triart Diol Hilic, 20*150 mm, 5 μm; Mobile Phase A: Water (0.1% NH₄HCO₃+0.1% NH₃—H₂O), Mobile Phase B: MeCN; Flow rate: 60 mL/min; Gradient: 15% B to 35% B in 10 min; Wave Length: 254/210 nm; RT: 10.38). The product-containing fractions were combined and evaporated partially in vacuo and lyophilized overnight to afford to obtain the title compound (ABH-4) (8.1 mg, 16 μmol, 8.1%) as a white solid. m/z 511.2 (M+H)⁺ (ES+). ¹H NMR (300 MHz, Methanol-d4) δ 8.20-8.07 (m, 2H), 8.02 (s, 1H), 7.87-7.73 (m, 2H), 7.52 (t, J=7.8 Hz, 1H), 7.10-7.01 (m, 1.0 Hz, 1H), 5.14 (s, 4H), 3.73-3.62 (m, 4H), 3.56 (t, J=6.5 Hz, 2H), 2.94 (s, 3H), 2.10-1.90 (m, 4H).

### Example 114: Synthesis of 2-(3-(2-(4-Methyl-4H-1,2,4-triazol-3-yl)ethyl)phenyl)-5-(trifluoromethyl)-[1,2,4]triazolo[1,5-a]pyridine (ABI-7)

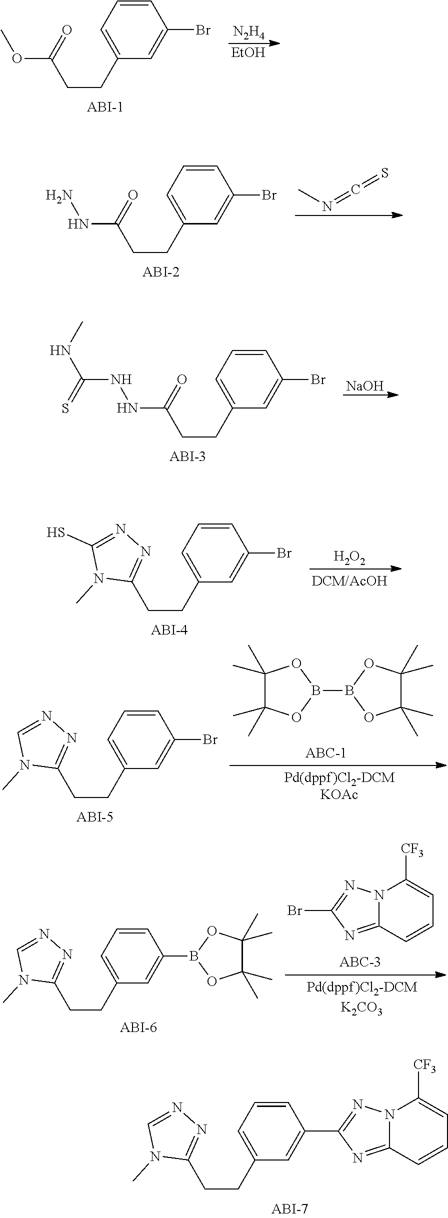

Step 1: 3-(3-Bromophenyl) propanehydrazide (ABI-2). To a stirred solution of methyl 3-(3-bromophenyl)propanoate (ABI-1) (200 mg, 1 Eq, 823 μmol) in EtOH (4 mL) was added hydrazine (255 mg, 98% Wt, 10 Eq, 8.23 mmol) at rt. The resulting mixture was stirred for 16 h at 50° C. The mixture was cooled to rt and concentrated in vacuo. The crude product was used in the next step directly without further purification. m/z 243.0/245.0 (M+H)⁺ (ES+)

Step 2: 2-(3-(3-Bromophenyl)propanoyl)-N-methylhydrazine-1-carbothioamide (ABI-3). To a stirred solution of the product from step 1 above (ABI-2) (200 mg, 1 Eq, 823 mmol) in THF (4 mL) was added methyl isothiocyanate (102 mg, 1.7 Eq, 1.40 mmol) at rt. The resulting mixture was stirred for 16 h at rt. The resulting mixture was concentrated in vacuo. The crude product was used in the next step directly without further purification. m/z 316.0/318.0 (M+H)⁺ (ES+).

Step 3: 5-(3-Bromophenethyl)-4-methyl-4H-1,2,4-triazole-3-thiol (ABI-4). To a stirred solution of the product from step 2 above (ABI-3) (384 mg, 1 Eq, 1.21 mmol) was added NaOH (4 mL, 1 molar) at rt. The resulting mixture was stirred for 16 h at rt. The resulting mixture was concentrated in vacuo. The residue was purified by Prep-TLC with DCM/EtOAc (10/1) to afford the sub-title compound (ABI-4) (300 mg, 1.01 mmol, 58%) as a yellow solid. m/z 298.0/300.0 (M+H)⁺ (ES+).

Step 4: 3-(3-Bromophenethyl)-4-methyl-4H-1,2,4-triazole (ABI-5). To a stirred solution of the product from step 3 above (ABI-4) (300 mg, 1 Eq, 1.01 mmol) in DCM (5 mL) were added AcOH (121 mg, 2 Eq, 2.01 mmol) and hydrogen peroxide (205 mg, 6 Eq, 6.04 mmol) dropwise at 0° C. The resulting mixture was stirred for 2 h at rt. The reaction was quenched with water at 0° C. The resulting mixture was diluted with water and extracted with DCM (3×50 mL). The combined organic layers were washed with brine (2×50 mL), dried (Na₂SO₄). After filtration, the filtrate was concentrated in vacuo. The crude product was purified by reverse flash column chromatography with the following conditions: Column, C18; mobile phase, Water (0.1% NH₄HCO₃) and MeCN (10% MeCN up to 50% in 10 min); Detector, UV 254/220 nm. This resulted in the sub-title compound (ABI-5) (250 mg, 939 μmol, 77%) as yellow oil. m/z 266.0/268.0 (M+H)⁺ (ES+).

Step 5: 4-Methyl-3-(3-(4,4,5,5-tetramethyl-1,3,2-dioxaborolan-2-yl)phenethyl)-4H-1,2,4-triazole (ABI-6). To a stirred solution of the product from step 4 above (ABI-5) (250 mg, 1 Eq, 939 μmol), intermediate (ABC-1) (286 mg, 1.2 Eq, 1.13 mmol) and KOAc (184 mg, 2 Eq, 1.88 mmol) in 1,4-dioxane (4 mL) at rt under nitrogen atmosphere. To the above mixture was added Pd(dppf)Cl₂-DCM (81 mg, 0.1 Eq, 100 μmol) at rt under nitrogen atmosphere. The resulting mixture was stirred for additional 16 h at 100° C. under nitrogen atmosphere. The mixture was cooled to rt and concentrated in vacuo. The residue was purified by Prep-TLC with DCM/MeOH (15/1) to afford the sub-title compound (ABI-6) (253 mg, 808 μmol, 86%) as yellow oil. m/z 314.2 (M+H)⁺ (ES+).

Step 6: 2-(3-(2-(4-Methyl-4H-1,2,4-triazol-3-yl)ethyl)phenyl)-5-(trifluoromethyl)-[1,2,4]triazolo[1,5-a]pyridine (ABI-7). To a stirred solution of the product from step 5 above (ABI-6) (253 mg, 1 Eq, 808 μmol), intermediate (ABC-3) (86 mg, 0.4 Eq, 323 μmol) and K₂CO₃ (335 mg, 3 Eq, 2.42 mmol) in 1,4-dioxane (4 mL) and water (1 mL) at rt. To the above mixture was added Pd(dppf)Cl₂-DCM (59 mg, 0.1 Eq, 81 μmol) at rt under nitrogen atmosphere. The resulting mixture was stirred for additional 16 h at 100° C. under nitrogen atmosphere. The mixture was cooled to rt and concentrated in vacuo. The residue was purified by Prep-TLC with DCM/MeOH (12/1). The crude product was purified by Prep-HPLC with the following conditions (Column: SunFire Prep C18 OBD Column, 19*150 mm, 5 μm; Mobile Phase A: Water(0.1% FA), Mobile Phase B: MeCN; Flow rate: 60 mL/min; Gradient: 10% B to 30% B in 10 min; Wave Length: 254/210 nm; RT: 10.38) to afford the title compound (ABI-7) (48.7 mg, 179 μmol, 16%) as a white solid. m/z 373.3 (M+H)⁺ (ES+). ¹H NMR (400 MHz, Methanol-d4) δ 8.34 (s, 1H), 8.20-8.11 (m, 2H), 8.09-8.04 (m, 1H), 7.87-7.79 (m, 1H), 7.73-7.69 (m, 1H), 7.52-7.45 (m, 1H), 7.41-7.37 (m, 1H), 3.55 (s, 3H), 3.28-3.16 (m, 4H).

### Example 115: Synthesis of (2-(3-(3-((4-Methyl-4H-1,2,4-triazol-3-yl)methyl)oxetan-3-yl)phenyl)-7-(trifluoromethyl)-1H-benzo[d]imidazol-5-yl)(5-azaspiro[2.4]heptan-5-yl)methanone (ABJ-2)

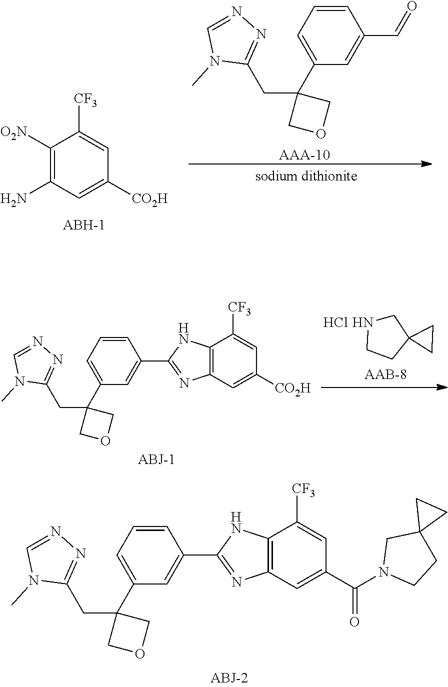

Step 1: 2-(3-(3-((4-Methyl-4H-1,2,4-triazol-3-yl)methyl)oxetan-3-yl)phenyl)-7-(trifluoromethyl)-1H-benzo[d]imidazole-5-carboxylic acid (ABJ-1). To a stirred mixture of intermediate (AAA-10) (450 mg, 1 Eq, 1.75 mmol) and intermediate (ABH-1) (481 mg, 1.1 Eq, 1.92 mmol) in EtOH (10 mL) was added a solution of sodium hydrosulfite (3.05 g, 10 Eq, 17.5 mmol) in water (10 mL) at rt. The resulting mixture was stirred for overnight at 80° C. under nitrogen atmosphere. The mixture was cooled down to rt and concentrated in vacuo. The residue was purified by reverse flash chromatography with the following conditions: (column, C18 silica gel; mobile phase, MeCN in water (0.1% FA), 10% to 80% gradient in 20 min; detector, UV 254/220 nm) to afford to afford the sub-title compound (ABJ-1) (300 mg, 656 μmol, 42%) as a brown solid. m/z 458.1 (M+H)⁺ (ES+).

Step 2: (2-(3-(3-((4-Methyl-4H-1,2,4-triazol-3-yl)methyl)oxetan-3-yl)phenyl)-7-(trifluoromethyl)-1H-benzo[d]imidazol-5-yl)(5-azaspiro[2.4]heptan-5-yl)methanone (ABJ-2). To a stirred mixture of the product from step 1 above (ABJ-1) (25 mg, 1 Eq, 55 μmol), intermediate (AAB-8) (5.3 mg, 1 Eq, 55 μmol) and HOBt (11 mg, 1.5 Eq, 83 μmol) in DMF (2 mL) was added DIPEA (21 mg, 3 Eq, 165 μmol) at rt. Then to this was added EDCI (16 mg, 1.5 Eq, 83 μmol). The resulting mixture was stirred for overnight at rt. The crude product was purified by reverse phase flash with the following conditions: (column, C18 silica gel; mobile phase, MeCN in water (0.1% NH₄HCO₃), 10% to 80% gradient in 20 min; detector, UV 254/220 nm) and Prep-HPLC with the following conditions: (Column: YMC-Triart Diol Hilic, 20*150 mm, 5 μm; Mobile Phase A: Water (0.1% NH₄HCO₃+0.1% NH₃—H₂O), Mobile Phase B: MeCN; Flow rate: 60 mL/min; Gradient: 25% B to 45% B in 10 min; Wave Length: 254/210 nm; RT: 10.38.). The product-containing fractions were combined and evaporated partially in vacuo and lyophilized overnight to afford the title compound (ABJ-2) (6.2 mg, 12 μmol, 21%) as a white solid. m/z 537.4 (M+H)⁺ (ES+). ¹H NMR (300 MHz, Methanol-d4) δ 8.21-7.99 (m, 3H), 7.87-7.74 (m, 2H), 7.59-7.48 (m, 1H), 7.08 (d, J=7.8 Hz, 1H), 5.15 (s, 4H), 3.90-3.68 (m, 4H), 3.53 (d, J=30.1 Hz, 2H), 2.95 (s, 3H), 2.01-1.83 (m, 2H), 0.79-0.53 (m, 4H).

### Example 116: Synthesis of 5-((5-Azaspiro[2.4]heptan-5-yl)methyl)-2-(3-(3-((4-methyl-4H-1,2,4-triazol-3-yl)methyl)oxetan-3-yl)phenyl)-7-(trifluoromethyl)-1H-benzo[d]imidazole (ABK-1)

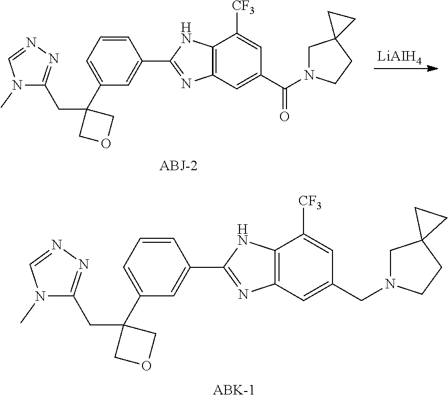

To a stirred solution of compound (ABJ-2) (125 mg, 1 Eq, 233 μmol) in THF (10 mL) was added a solution of LiAlH₄ in THF (700 μL, 1 molar, 3 Eq, 699 μmol) at 0° C. under nitrogen atmosphere. The resulting mixture was stirred for 3 h at rt under nitrogen atmosphere. The reaction was quenched by the addition of sodium sulfate decahydrate and diluted with water (50 mL) at 0° C. The resulting mixture was diluted with water and extracted with EtOAc (3×20 mL). The combined organic layers were washed with brine (2×20 mL), dried (Na₂SO₄). After filtration, the filtrate was concentrated in vacuo. The crude product was purified by reverse phase flash with the following conditions: (column, C18 silica gel; mobile phase, MeCN in water (0.1% NH₄HCO₃), 10% to 80% gradient in 20 min; detector, UV 254/220 nm) and Prep-HPLC with the following conditions: (Column: YMC-Triart Diol Hilic, 20*150 mm, 5 μm; Mobile Phase A: Water (0.1% NH₄HCO₃+0.1% NH₃—H₂O), Mobile Phase B: MeCN; Flow rate: 60 mL/min; Gradient: 25% B to 45% B in 10 min; Detector, 254/220 nm; RT: 9.28). The product-containing fractions were combined and evaporated partially in vacuo and lyophilized overnight to afford the title compound (ABK-1) (28.1 mg, 54 μmol, 23%) as a white solid. m/z 523.2 (M+H)⁺ (ES+). ¹H NMR (400 MHz, Methanol-d4) δ 8.18 (s, 1H), 8.13-8.06 (m, 1H), 7.87-7.79 (m, 2H), 7.63 (s, 1H), 7.51 (t, J=7.8 Hz, 1H), 7.07-7.01 (m, 1H), 5.19-5.11 (m, 4H), 3.87 (s, 2H), 3.71 (s, 2H), 2.93 (s, 3H), 2.86 (t, J=7.0 Hz, 2H), 2.59 (s, 2H), 1.90 (t, J=7.0 Hz, 2H), 0.65-0.54 (m, 4H).

### Example 117: Synthesis of 2-(3-(3-((4-Methyl-4H-1,2,4-triazol-3-yl)methyl)oxetan-3-yl) phenyl)-5-(pyrrolidin-1-ylmethyl)-7-(trifluoromethyl)-1H-benzo[d]imidazole (ABL-1)

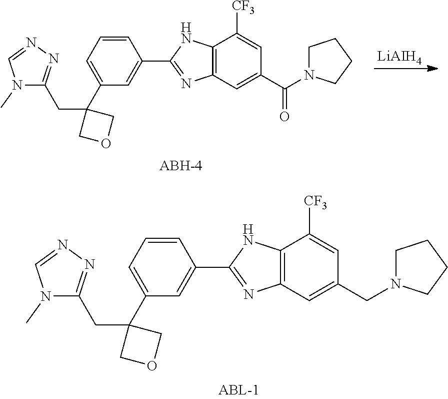

To a stirred solution of compound (ABH-4) (165 mg, 1 Eq, 323 μmol) in THF (10 mL) was added a solution of LiAlH₄ in THF (970 mL, 1 molar, 3 Eq, 969 μmol) at 0 degrees C. under nitrogen atmosphere. The resulting mixture was stirred for 3 h at rt under nitrogen atmosphere. The reaction was quenched by the addition of sodium sulfate decahydrate and diluted with water (50 mL) at 0° C. The resulting mixture was diluted with water and extracted with EtOAc (3×20 mL). The combined organic layers were washed with brine (2×20 mL), dried (Na₂SO₄). After filtration, the filtrate was concentrated in vacuo. The crude product was purified by reverse phase flash with the following conditions: (column, C18 silica gel; mobile phase, MeCN in water (0.1% NH₄HCO₃), 10% to 80% gradient in 20 min; detector, UV 254/220 nm) and Prep-HPLC with the following conditions: (Column: YMC-Triart Diol Hilic, 20*150 mm, 5 μm; Mobile Phase A: Water (0.1% NH₄HCO₃+0.1% NH₃—H₂O), Mobile Phase B: MeCN; Flow rate: 60 mL/min; Gradient: 25% B to 45% B in 10 min; Detector, 254/220 nm; RT: 9.28). The product-containing fractions were combined and evaporated partially in vacuo and lyophilized overnight to afford the title compound (ABL-1) (19.4 mg, 39 μmol, 12%) as a white solid. m/z 497.1 (M+H)⁺ (ES+). ¹H NMR (300 MHz, Methanol-d4) δ 8.16 (s, 1H), 8.08 (d, J=8.0 Hz, 1H), 7.86-7.77 (m, 2H), 7.61 (s, 1H), 7.50 (t, J=7.8 Hz, 1H), 7.08-6.99 (m, 1H), 5.14 (s, 4H), 3.85 (s, 2H), 3.69 (s, 2H), 2.93 (s, 3H), 2.63 (s, 4H), 1.91-1.81 (m, 4H).

### Example 118: Synthesis of 7-Chloro-2-(3-(3-((4-methyl-4H-1,2,4-triazol-3-yl)methyl) oxetan-3-yl)phenyl)-1H-benzo[d]imidazole (ABM-2)

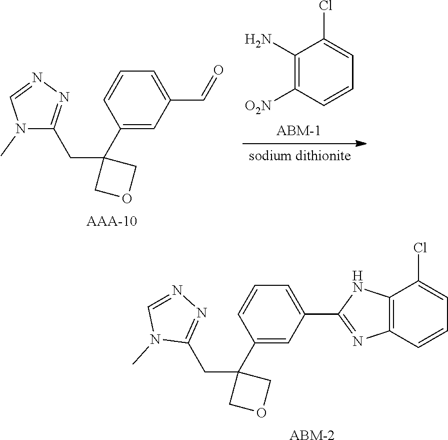

To a stirred solution of intermediate (AAA-10) (50 mg, 1 Eq, 194 μmol) and 2-chloro-6-nitroaniline (ABM-1) (34 mg, 1 Eq, 194 μmol) in EtOH (1 mL) was added a solution of disodium hydrosulfite (169 mg, 5 Eq, 972 μmol) in water (1 mL). The resulting mixture was stirred overnight dropwise at 80° C. under nitrogen atmosphere. The mixture was cooled down to rt and concentrated in vacuo. The crude product was purified by reverse flash column chromatography with the following conditions: Column, C18; mobile phase, Water (0.1% NH₄HCO₃) and MeCN (10% MeCN up to 80% in 20 min); Detector, UV 254/220 nm) and Prep-HPLC with the following conditions: Column: SunFire Prep C18 OBD Column, 19*150 mm, 5 m 10 nm; Mobile Phase A: Water (0.1% NH₄HCO₃), Mobile Phase B: MeCN; Flow rate: 60 mL/min; Gradient: 35% B to 52% B in 10 min; UV detection at 254/210 nm; RT: 5.85. The product-containing fractions were combined and evaporated partially in vacuo and lyophilized overnight to afford the title compound (ABM-2) (32.9 mg, 87 μmol, 44%) as a white solid. m/z 380.0/382.0 (M+H)⁺ (ES+). ¹H NMR (400 MHz, DMSO-d6) δ 13.16 (s, 1H), 8.19 (s, 1H), 8.09 (s, 1H), 7.87 (s, 1H), 7.59-7.43 (m, 2H), 7.32-7.17 (m, 2H), 7.11-7.06 (m, 1H), 5.01 (d, J=6.1 Hz, 2H), 4.96 (d, J=6.1 Hz, 2H), 3.57 (s, 2H), 2.95 (s, 3H).

### Example 119: Synthesis of 2-(3-(1-Methyl-4-(4-methyl-4H-1,2,4-triazol-3-yl)-1H-pyra zol-5-yl)phenyl)-5-(trifluoromethyl)-[1,2,4]triazolo[1,5-a]pyridine (ABN-2)

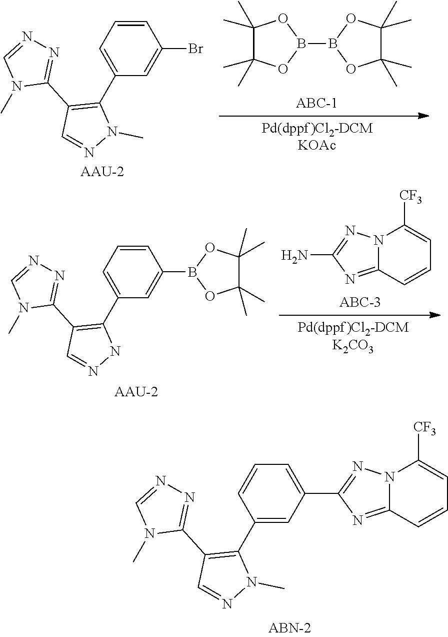

Step 1: 4-Methyl-3-(1-methyl-5-(3-(4,4,5,5-tetramethyl-1,3,2-dioxaborolan-2-yl)phenyl)-1H-pyrazol-4-yl)-4H-1,2,4-triazole (ABN-1). To a stirred solution of intermediate (AAU-2) (100 mg, 1 Eq, 314 μmol), bis(pinacolato)diboron (ABC-1) (88 mg, 1.1 Eq, 345 μmol) and KOAc (92 mg, 3 Eq, 942 μmol) in dioxine (4 mL) and water (1 mL) was added and Pd(dppf)Cl₂-DCM (23 mg, 0.1 Eq, 31 μmol) at 100° C. under nitrogen atmosphere. The resulting mixture was cooled to rt and concentrated in vacuo. The crude product was purified by reverse flash column chromatography with the following conditions: Column, C18; mobile phase, Water (0.1% NH₄HCO₃) and MeCN (10% ACN up to 50% in 10 min); Detector, UV 254/220 nm to afford the sub-title compound (ABN-1) (60 mg, 164 μmol, 52%) as a yellow solid. m/z 366.2 (M+H)⁺ (ES+).

Step 2: 4-Methyl-3-(1-methyl-5-{3-[5-(trifluoromethyl)-[1,2,4]triazolo[1,5-a]pyridin-2-yl]phenyl}pyrazol-4-yl)-1,2,4-triazole (ABN-2). To a stirred solution of the product from step 1 above (ABN-1) (60 mg, 1 Eq, 164 μmol), intermediate (ABC-3) (48 mg, 1.1 Eq, 180 μmol) and K₂CO₃ (68 mg, 3 Eq, 492 μmol) in dioxine (4 mL) and water (1 mL) was added Pd(dppf)Cl₂-DCM (12.02 mg, 0.016 mmol, 0.1 Eq) at 100° C. under nitrogen atmosphere. The resulting mixture was cooled to rt and concentrated in vacuo. The crude product was purified by Prep-HPLC with the following conditions (Column: YMC-Actus Triart C18 ExRS, 30*250 mm, 5 μm; Mobile Phase A: Water(0.1% NH₄HCO₃+0.1% NH₃·H₂O), Mobile Phase B: MeCN; Flow rate: 60 mL/min; Gradient: 22% B to 52% B in 7 min; Wave Length: 254/210 nm; RT: 5.32) to afford the title compound (ABN-2) (21 mg, 50 μmol, 30%) as a white solid. m/z 425.1 (M+H)⁺ (ES+). ¹H NMR (300 MHz, DMSO-d6) δ 8.41 (s, 1H), 8.31-8.26 (m, 1H), 8.25-8.20 (m, 1H), 8.16 (t, J=1.8 Hz, 1H), 7.93 (s, 1H), 7.89-7.80 (m, 2H), 7.72-7.60 (m, 2H), 3.86 (s, 3H), 3.42 (s, 3H).

### Example 120: Synthesis of 3-(3-(3-((4-Methyl-4H-1,2,4-triazol-3-yl)methyl)oxetan-3-yl) phenyl)-5-(trifluoromethyl)pyridin-2(1H)-one (ABO-1)

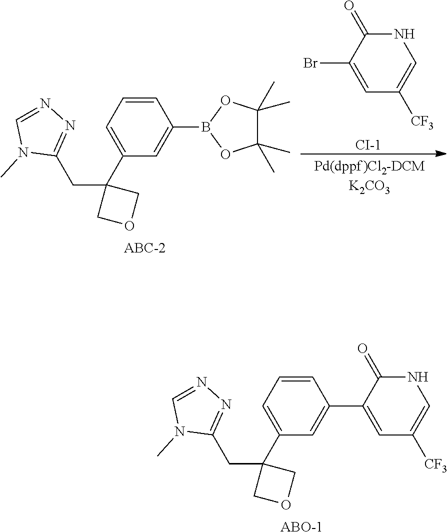

To a stirred mixture of intermediate (ABC-2) (100 mg, 1 Eq, 281 μmol), 3-bromo-5-(trifluoromethyl)pyridin-2-ol (CI-1) (82 mg, 1.2 Eq, 337 μmol) and K₂CO₃ (117 mg, 3 Eq, 843 μmol) in 1,4-dioxane (4 mL) and water (1 mL) at rt under nitrogen atmosphere. To the above mixture was added Pd(dppf)Cl₂-DCM (21 mg, 0.1 Eq, 28 μmol) at rt under nitrogen atmosphere. The resulting mixture was stirred for additional 16 h at 100° C. under nitrogen atmosphere. The mixture was allowed to cool down to rt and concentrated in vacuo. The residue was purified by Prep-TLC with DCM/MeOH (12/1). The crude product was purified by Prep-HPLC with the following conditions (Column: YMC-Actus Triart C18 ExRS, 30*250, 5 um; Mobile Phase A: Water (0.1% NH₄HCO₃+0.1% NH₃·H₂O), Mobile Phase B: MeCN; Flow rate: 60 mL/min; Gradient: 20 B to 33 B in 7 min; Detector, UV 254/210 nm; RT: 6.32) to afford the title compound (ABO-1) (9.6 mg, 25 μmol, 8.7%) as a white solid. m/z 391.1 (M+H)⁺ (ES+). ¹H NMR (400 MHz, Methanol-d4) δ 8.18 (s, 1H), 7.90-7.85 (m, 1H), 7.70 (d, J=2.7 Hz, 1H), 7.57-7.53 (m, 1H), 7.38 (t, J=7.8 Hz, 1H), 7.17 (t, J=1.8 Hz, 1H), 6.88-6.82 (m, 1H), 5.08 (d, J=0.9 Hz, 4H), 3.66 (s, 2H), 2.85 (s, 3H).

### Example 121: Synthesis of 4-Bromo-2-(3-[3-[(4-methyl-1,2,4-triazol-3-yl)methyl]oxetan-3-yl]phenyl)-1H-1,3-benzodiazole (ABP-2)

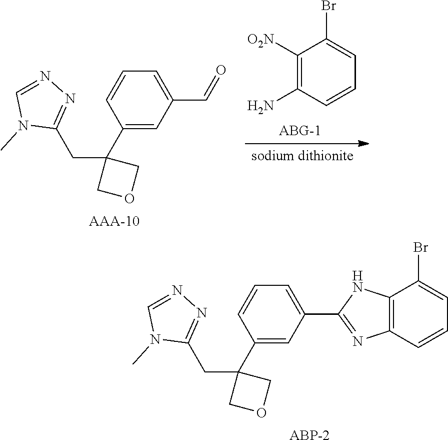

To a stirred solution of intermediate (AAA-10) (50 mg, 1 Eq, 190 μmol) and 3-bromo-2-nitroaniline (ABG-1) (46 mg, 1.1 Eq, 210 μmol) in EtOH (2 mL) was added sodium dithionite (169 mg, 5 Eq, 970 μmol) at rt. The resulting mixture was stirred for overnight at 80° C. under nitrogen atmosphere. The mixture was cooled to rt and concentrated in vacuo. The crude product was purified by reverse flash column chromatography with the following conditions: Column, C18; mobile phase, Water (0.1% NH₄HCO₃) and ACN (10% MeCN up to 70% in 20 min); Detector, UV 254/220 nm. The product-containing fractions were combined and concentrated in vacuo. The crude product was purified by Prep-HPLC with the following conditions: Column: YMC-Triart Diol Hilic, 20*150 mm 5 um; Mobile Phase A: Water (0.1% NH₄HCO₃+0.1% NH₃·H₂O), Mobile Phase B: MeCN; Flow rate: 60 mL/min; Gradient: 15 B to 45 B in 10 min; Detector, UV 254/210 nm; RT: 10.38. The product-containing fractions were combined and evaporated partially in vacuo and lyophilized overnight to afford the title compound (ABP-2) (36.9 mg, 87 μmol, 44%) as a white solid. m/z 424.0/426.0 (M+H)⁺ (ES+). ¹H NMR (300 MHz, Methanol-d4) δ 8.17 (s, 1H), 8.06 (d, J=7.8 Hz, 1H), 7.81 (t, J=1.8 Hz, 1H), 7.55-7.41 (m, 3H), 7.19 (t, J=7.9 Hz, 1H), 7.03 (d, J=7.8 Hz, 1H), 5.14 (d, J=1.2 Hz, 4H), 3.70 (s, 2H), 2.92 (s, 3H).

### Example 122: Synthesis of 2-(3-(3-((4-Methyl-4H-1,2,4-triazol-3-yl)methyl)oxetan-3-yl) phenyl)-7-(trifluoromethoxy)-1H-benzo[d]imidazole (ABQ-2)

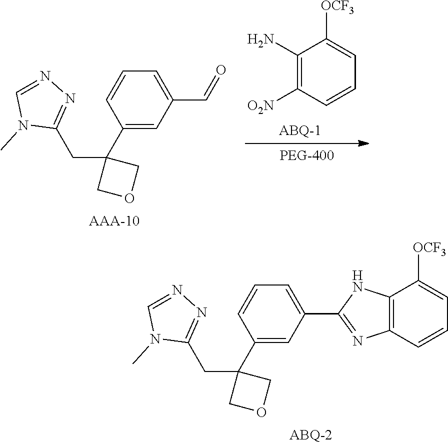

To a stirred mixture of intermediate (AAA-10) (50 mg, 1 Eq, 194 μmol) and 3-(trifluoromethoxy)benzene-1,2-diamine (ABQ-1) (41 mg, 1.1 Eq, 214 μmol) in EtOH (0.8 mL) was added PEG-400 (0.04 mL) dropwise at rt under nitrogen atmosphere. The resulting mixture was stirred for overnight at 80° C. under nitrogen atmosphere. The mixture was allowed to cool down to rt and concentrated in vacuo. The residue was purified by Prep-TLC with DCM/MeOH (15/1) and Prep-HPLC with the following conditions: Column: Xselect CSH C18 OBD Column 30*150 mm 5 μm, n; Mobile Phase A: Water(0.05% FA), Mobile Phase B: MeCN; Flow rate: 60 mL/min; Gradient: 22% B to 44% B in 7 min; Wave Length: 254/210 nm; RT: 5.92. The product-containing fractions were combined and evaporated partially in vacuo and lyophilized overnight to afford the title compound (ABQ-2) (24.4 mg, 57 μmol, 29%) as an off-white solid. m/z 430.0 (M+H)⁺ (ES+). ¹H NMR (400 MHz, Methanol-d4) δ 8.19 (s, 1H), 8.10-8.05 (m, 1H), 7.80 (t, J=1.8 Hz, 1H), 7.59 (d, J=8.0 Hz, 1H), 7.51 (d, J=7.8 Hz, 1H), 7.33 (t, J=8.1 Hz, 1H), 7.25-7.18 (m, 1H), 7.07-7.03 (m, 1H), 5.18-5.11 (m, 4H), 3.71 (s, 2H), 2.94 (s, 3H).

### Example 123: Synthesis of (S)-(3-Hydroxypyrrolidin-1-yl)(2-(3-(3-((4-methyl-4H-1,2,4-triazol-3-yl)methyl)oxetan-3-yl)phenyl)-7-(trifluoromethyl)-1H-benzo[d]imidazol-5-yl)methanone (ABR-2)

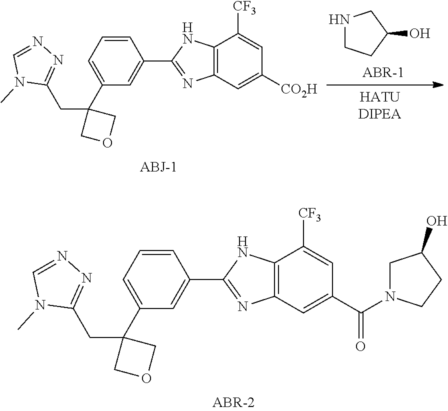 [SMILES: Cn1cnnc1CC1(c2cccc(-c3nc4cc(C(=O)O)cc(C(F)(F)F)c4[nH]3)c2)COC1 ; O[C@H]1CCNC1 ; Cn1cnnc1CC1(c2cccc(-c3nc4cc(C(=O)N5CC[C@H](O)C5)cc(C(F)(F)F)c4[nH]3)c2)COC1]

Into an 8 mL sealed tube were added intermediate (ABJ-1) (40 mg, 1 Eq, 87 μmol), (3S)-pyrrolidin-3-ol (ABR-1) (9 mg, 1.2 Eq, 104 μmol) and DIPEA (34 mg, 3 Eq, 261 μmol) in DMF (2 mL) at rt. To the above mixture was added HATU (50 mg, 1.5 Eq, 131 μmol) over 3 min at 0° C. The resulting mixture was stirred for additional 2 h at rt. The crude product was purified by Prep-HPLC with the following conditions: Column: SunFire Prep C18 OBD Column, 19×150 mm, 5 μm; Mobile Phase A: Water (0.1% NH₄HCO₃+0.1% NH₃·H₂O), Mobile Phase B: MeCN; Flow rate: 60 mL/min; Gradient: 30 B to 60 B in 7 min; Detector, UV 254/210 nm; RT: 6.08 to afford the title compound (ABR-2) (19.9 mg, 38 μmol, 43%) as a white solid. m/z 527.4 (M+H)⁺ (ES+). ¹H NMR (400 MHz, Methanol-d4) δ 8.10-7.84 (m, 3H), 7.70 (d, J=19.5 Hz, 2H), 7.41 (t, J=7.8 Hz, 1H), 6.99-6.93 (m, 1H), 5.03 (d, J=1.3 Hz, 4H), 4.52-4.27 (m, 1H), 3.74-3.63 (m, 2H), 3.63-3.41 (m, 3H), 3.31 (d, J=11.4 Hz, 1H), 2.83 (s, 3H), 2.12-1.81 (m, 2H).

### Example 124: Synthesis of (R)-(3-Hydroxypyrrolidin-1-yl)(2-(3-(3-((4-methyl-4H-1,2,4-triazol-3-yl)methyl)oxetan-3-yl)phenyl)-7-(trifluoromethyl)-1H-benzo[d]imidazol-5-yl)methanone (ABS-2)

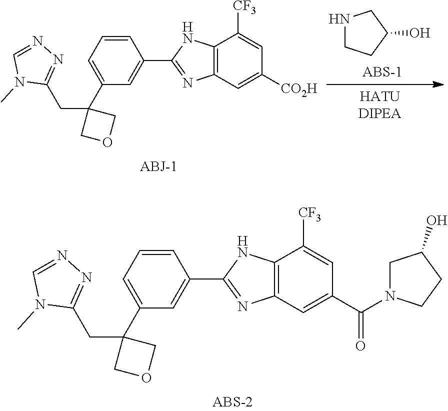 [SMILES: Cn1cnnc1CC1(c2cccc(-c3nc4cc(C(=O)O)cc(C(F)(F)F)c4[nH]3)c2)COC1 ; O[C@@H]1CCNC1 ; Cn1cnnc1CC1(c2cccc(-c3nc4cc(C(=O)N5CC[C@@H](O)C5)cc(C(F)(F)F)c4[nH]3)c2)COC1]

To a stirred solution of intermediate (ABJ-1) (50 mg, 1 Eq, 100 mmol) in DMF (2 mL) were added (3R)-pyrrolidin-3-ol (ABS-1) (11 mg, 1.2 Eq, 130 μmol), DIPEA (42 mg, 3 Eq, 320 mmol) and HATU (62 mg, 1.5 Eq, 160 mmol) at 0° C. The resulting mixture was stirred for 30 min at rt. The resulting mixture was concentrated in vacuo. The crude product was purified by reverse flash column chromatography with the following conditions: Column, C18; mobile phase, Water (0.1% NH4HCO3) and MeCN (10% MeCN up to 60% in 15 min); Detector, UV 254/220 nm. The product-containing fractions were combined and concentrated in vacuo. The crude product was purified by Prep-HPLC with the following conditions: Column: SunFire Prep C18 OBD Column, 19×150 mm, 5 μm; Mobile Phase A: Water (0.1% NH4HCO3+0.1% NH3·H2O), Mobile Phase B: MeCN; Flow rate: 60 mL/min; Gradient: 30 B to 60 B in 7 min; Detector, UV 254/210 nm; RT: 6.08. This resulted in the title compound (ABS-2) (15.4 mg, 29 μmol, 26%) as a white solid. m/z 527.2 (M+H)+ (ES+). ¹H NMR (400 MHz, Methanol-d4) δ 8.20-8.10 (m, 2H), 8.04 (d, J=7.0 Hz, 1H), 7.87-7.77 (m, 2H), 7.53 (t, J=7.8 Hz, 1H), 7.10-7.06 (m, 1H), 5.15 (d, J=1.3 Hz, 4H), 4.48 (d, J=49.6 Hz, 1H), 3.86-3.56 (m, 5H), 3.43 (d, J=11.4 Hz, 1H), 2.95 (s, 3H), 2.21-1.95 (m, 2H).

### Example 125: Synthesis of 2-(3-(3-((4-Methyl-4H-1,2,4-triazol-3-yl)methyl)oxetan-3-yl) phenyl)-7-(trifluoromethyl)benzo[d]thiazole (ABT-2)

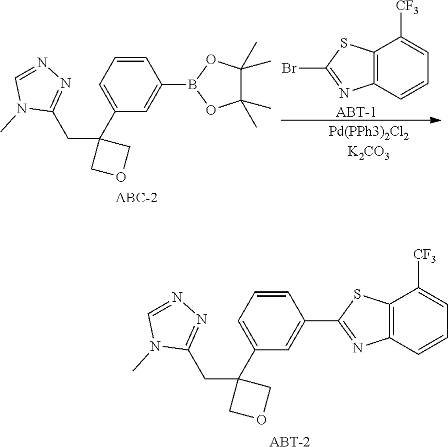

To a solution of 2-bromo-7-(trifluoromethyl)-1,3-benzothiazole (ABT-1) (50 mg, 1 Eq, 177 μmol), intermediate (ABC-2) (63 mg, 1 Eq, 170 μmol) and K₂CO₃ (74 mg, 3 Eq, 530 μmol) in toluene (5 mL), EtOH (1 mL) and water (0.5 mL) was added Pd(PPh₃)₂Cl₂ (25 mg, 0.2 Eq, 35 μmol) at rt under nitrogen atmosphere. After stirring overnight at 80° C. under nitrogen atmosphere, the mixture was cooled down to rt and concentrated in vacuo. The crude product was purified by reverse flash column chromatography with the following conditions: Column, C18; mobile phase, Water (0.1% NH₄HCO₃) and MeCN (10% MeCN up to 80% in 20 min); Detector, UV 254/220 nm and Prep-HPLC with the following conditions: Column: Sunfire prep C18 column, 30*150 mm, 5 μm; Mobile Phase A: Water(0.1% FA), Mobile Phase B: MeCN; Flow rate: 60 mL/min; Gradient: 36% B to 47% B in 9 min; Wave Length: 254/220 nm; RT: 8.68. The product-containing fractions were combined and evaporated partially in vacuo and lyophilized overnight to afford the title compound (ABT-2) (37.4 mg, 87 μmol, 49%) as a white solid. m/z 431.3 (M+H)⁺ (ES+). ¹H NMR (400 MHz, Methanol-d4) δ 8.19-8.15 (m, 1H), 8.08 (s, 1H), 7.97-7.93 (m, 1H), 7.72-7.69 (m, 1H), 7.65-7.59 (m, 1H), 7.57 (t, J=1.8 Hz, 1H), 7.42 (t, J=7.8 Hz, 1H), 7.06-7.03 (m, 1H), 5.05-4.98 (m, 4H), 3.59 (s, 2H), 2.84 (s, 3H).

### Example 126: Synthesis of 2-(2′-(4-Methyl-4H-1,2,4-triazol-3-yl)-[1,1′-biphenyl]-3-yl)-5-(trifluoromethyl)-[1,2,4]triazolo[1,5-a]pyridine (ABU-2)

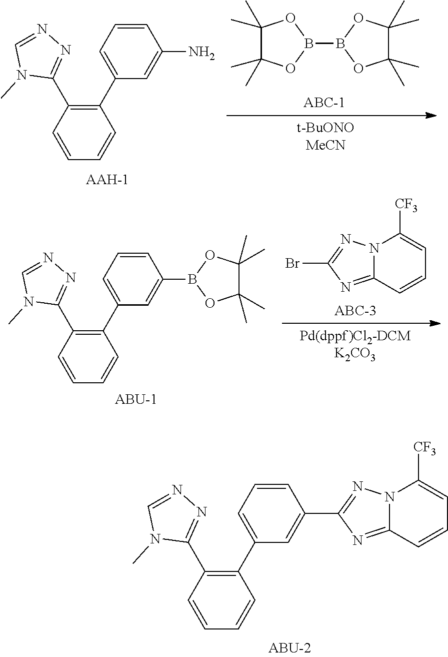

Step 1: 4-Methyl-3-[3-(4,4,5,5-tetramethyl-1,3,2-dioxaborolan-2-yl)-[1,1-biphenyl]-2-yl]-1,2,4-triazole (ABU-1). To a stirred solution of intermediate (AAH-1) (300 mg, 1 Eq, 1.19 mmol) and bis(pinacolato)diboron (ABC-1) (457 mg, 1.5 Eq, 1.79 mmol) in MeCN (10.0 mL) was added tert-butyl nitrite (309 mg, 2.5 Eq, 2.99 mmol) at rt. The resulting solution was stirred for overnight at 80° C. under nitrogen atmosphere. The mixture was cooled to rt and concentrated in vacuo. The crude product was purified by reverse flash column chromatography with the following conditions: Column, C18; mobile phase, Water (0.1% FA) and MeCN (10% MeCN up to 48% in 10 min); Detector, UV 254/220 nm. The product-containing fractions were combined and concentrated in vacuo. This resulted in the sub-title compound (ABU-1) (200 mg, 554 μmol, 46%). m/z 362.2 (M+H)⁺ (ES+).

Step 2: 4-Methyl-3-[3-[5-(trifluoromethyl)-[1,2,4]triazolo[1,5-a]pyridin-2-yl]-[1,1-biphenyl]-2-yl]-1,2,4-triazole (ABU-2). To a solution of intermediate (ABC-3) (40 mg, 1 Eq, 150 mmol), the product from step 1 above (ABU-1) (60 mg, 1.1 Eq, 160 mmol) and K₂CO₃ (62 mg, 3 Eq, 450 mmol) in 1.4-dioxane (4 mL) and water (1 mL) was added and Pd(dppf)Cl₂-DCM (11 mg, 0.1 Eq, 10 mmol) at rt under nitrogen atmosphere. The resulting mixture was stirred for additional 2 hours at 80° C. under nitrogen atmosphere. The mixture was cooled to rt, diluted with water and extracted with EtOAc (3×20 mL). The combined organic layers were washed with brine (2×20 mL), dried (Na₂SO₄). After filtration, the filtrate was concentrated in vacuo. The residue was purified by Prep-TLC with EtOAc/petroleum ether (1/1) and concentrated in vacuo. The crude product was purified by Prep-HPLC with the following conditions: Column: Sunfire prep C18 column, 30*150, 5 um; Mobile Phase A: Water (0.1% FA), Mobile Phase B: MeCN; Flow rate: 60 mL/min; Gradient: 28 B to 48 B in 9 min; Detector, UV 254/210 nm; RT: 8.38. The product-containing fractions were combined and evaporated partially in vacuo and lyophilized overnight to afford the title compound (ABU-2) (18.5 mg, 44 μmol, 29%) as a white solid. m/z 421.3 (M+H)⁺ (ES+). ¹H NMR (300 MHz, Methanol-d4) δ 8.35 (s, 1H), 8.28-8.16 (m, 2H), 8.06 (d, J=8.9 Hz, 1H), 7.86-7.58 (m, 6H), 7.50 (t, J=7.8 Hz, 1H), 7.35-7.30 (m, 1H), 3.20 (s, 3H).

### Example 127: Synthesis of 3-(2′-(4-Methyl-4H-1,2,4-triazol-3-yl)-[1,1′-biphenyl]-3-yl)-5-(trifluoromethyl)pyridin-2(1H)-one (ABV-1)

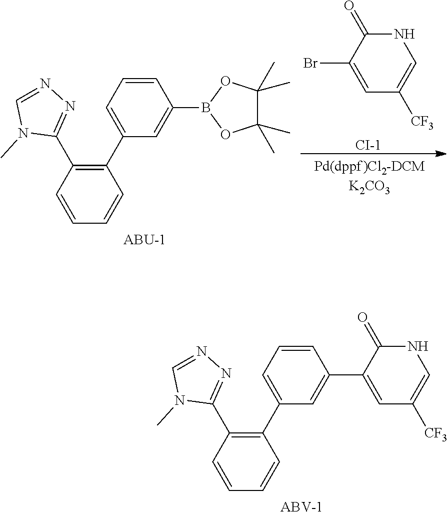

To a stirred solution of intermediate (ABU-1) (61 mg, 1 Eq, 230 μmol), 3-bromo-5-(trifluoromethyl)pyridin-2-ol (CI-1) (60 mg, 1.1 Eq, 250 mmol) and K₂CO₃ (94 mg, 3 Eq, 700 mmol) in 1,4-dioxane (5 mL) and water (1 mL) was added Pd(dppf)Cl₂-DCM (33 mg, 0.2 Eq, 50 mmol) at 80° C. under nitrogen atmosphere. The resulting mixture was stirred for 2 h at 80° C. under nitrogen atmosphere. The mixture was cooled to rt, diluted with water and extracted with DCM (3×20 mL). The combined organic layers were washed with brine (2×20 mL), dried (Na₂SO₄). After filtration, the filtrate was concentrated in vacuo. The crude product was purified by Prep-HPLC with the following conditions (Column: Sunfire prep C18 column, 30*150, 5 um; Mobile Phase A: Water (0.1% FA), Mobile Phase B: MeCN; Flow rate: 60 mL/min; Gradient: 22 B to 42 B in 9 min; Detector, UV 254/220 nm; RT1:8.6) to afford the title compound (ABV-1) (12.5 mg, 32 μmol, 14%) as a white solid. m/z 397.3 (M+H)⁺ (ES+). ¹H NMR (400 MHz, Methanol-d4) δ 8.37 (s, 1H), 7.89-7.85 (m, 1H), 7.79-7.69 (m, 2H), 7.69-7.56 (m, 4H), 7.53 (t, J=1.8 Hz, 1H), 7.41 (t, J=7.8 Hz, 1H), 7.25-7.20 (m, 1H), 3.12 (s, 3H).

### Example 128: Synthesis of 2-(2′-(4-Methyl-4H-1,2,4-triazol-3-yl)-[1,1′-biphenyl]-3-yl)-7-(trifluoromethyl)-1H-benzo[d]imidazole (ABW-1)

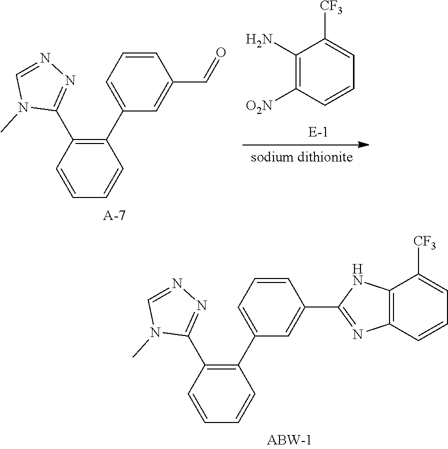

Into a 25-mL round-bottom flask, was placed intermediate (A-7) (55 mg, 1 Eq, 209 μmol) and intermediate (E-1) (86 mg, 2 Eq, 418 mmol) in EtOH (2 mL) and water (2 mL), then sodium hydrosulfite (364 mg, 10 Eq, 2.09 mmol) was added at rt. The resulting solution was stirred overnight at 80° C. The mixture was cooled to rt and concentrated in vacuo. The crude product was purified by Prep-HPLC with the following conditions: Column: Sunfire prep C18 column, 30×150 mm, 5 μm; Mobile Phase A: Water (0.1% FA), Mobile Phase B: MeCN; Flow rate: 60 mL/min; Gradient: 35 B to 55 B in 8 min; Detector, UV 220/254 nm; RT: 6.52. This resulted in the title compound (ABW-1) (38.6 mg, 92 μmol, 44%) as a white solid. m/z 420.3 (M+H)⁺ (ES+). ¹H NMR (300 MHz, Methanol-d4) δ 8.37 (s, 1H), 8.22-8.10 (m, 2H), 7.90-7.73 (m, 3H), 7.72-7.57 (m, 3H), 7.54-7.37 (m, 2H), 7.28-7.21 (m, 1H), 3.20 (s, 3H).

### Example 129: Synthesis of 5-Cyclopropyl-3-(3-(3-((4-methyl-4H-1,2,4-triazol-3-yl)methyl)oxetan-3-yl)phenyl)pyridin-2(1H)-one (ABX-3)

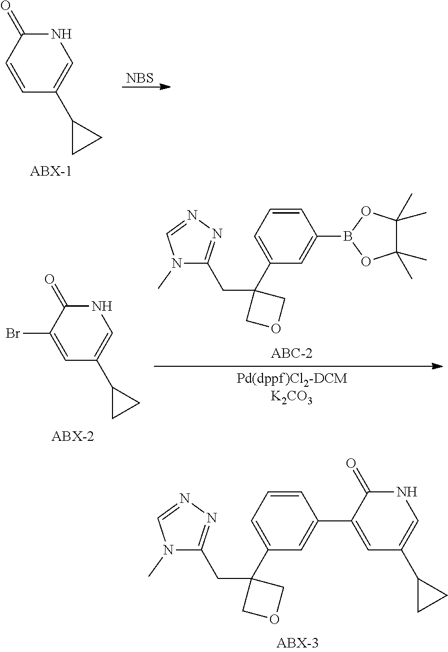

Step 1: 3-Bromo-5-cyclopropylpyridin-2(1H)-one (ABX-2). To a stirred solution of 5-cyclopropyl-1H-pyridin-2-one (ABX-1) (15 mg, 1 Eq, 1.11 mmol) in DMF (5 mL) was added NBS (257 mg, 1.3 Eq, 1.44 mmol) at rt under nitrogen atmosphere. The resulting mixture was stirred overnight at rt under nitrogen atmosphere. The crude product was purified by reverse flash column chromatography with the following conditions: Column, C18; mobile phase, Water (0.1% NH4HCO3) and MeCN (0% ACN up to 60% in 20 min); Detector, UV 254/220 nm to afford the sub-title compound (ABX-2) (100 mg, 469 μmol, 42%) as a brown solid. m/z 214.0/216.0 (M+H)⁺ (ES+).

Step 2: 5-Cyclopropyl-3-(3-(3-((4-methyl-4H-1,2,4-triazol-3-yl)methyl)oxetan-3-yl)phenyl)pyridin-2(1H)-one (ABX-3). To a solution of the product from step 1 above (ABX-2) (50 mg, 1 Eq, 230 mmol), intermediate (ABC-2) (83 mg, 1 Eq, 230 mmol) and K₂CO₃ (97 mg, 3 Eq, 700 mmol) in 1,4-dioxane (4 mL) and water (0.4 mL) was added Pd(dppf)Cl₂-DCM (34 mg, 0.2 Eq, 47 mmol) at rt under nitrogen atmosphere. After stirring for 12 h at 80° C. under a nitrogen atmosphere, the mixture was cooled down to rt and concentrated in vacuo. The crude product was purified by reverse flash column chromatography with the following conditions: Column, C18; mobile phase, Water (0.1% FA) and MeCN (10% MeCN up to 80% in 20 min); Detector, UV 254/220 nm and Prep-HPLC with the following conditions: (Column: XBridge Prep OBD C18 Column, 30*150 mm, 5 μm; Mobile Phase A: Water (0.1% NH₄HCO₃+0.1% NH₃—H₂O), Mobile Phase B: MeCN; Flow rate: 60 mL/min; Gradient: 10% B to 40% B in 7 min; Wave Length: 254/210 nm; RT: 5.9). The product-containing fractions were combined and evaporated partially in vacuo and lyophilized overnight to afford the title compound (ABX-3) (32.2 mg, 89 μmol, 36%) as a white solid. m/z 363.1 (M+H)⁺ (ES+). ¹H NMR (400 MHz, Methanol-d4) δ 8.09 (s, 1H), 7.45-7.40 (m, 1H), 7.29 (d, J=2.6 Hz, 1H), 7.24 (t, J=7.7 Hz, 1H), 7.10-7.07 (m, 1H), 7.00 (t, J=1.8 Hz, 1H), 6.71-6.66 (m, 1.1 Hz, 1H), 4.97 (s, 4H), 3.55 (s, 2H), 2.74 (s, 3H), 1.77-1.66 (m, 1H), 0.88-0.76 (m, 2H), 0.57-0.48 (m, 2H).

### Example 130: Synthesis of 2-(3-(3-((4-Methyl-4H-1,2,4-triazol-3-yl)methyl)oxetan-3-yl)phenyl)-4-(trifluoromethyl)-1H-benzo[d]imidazole-6-carboxylic Acid (ABY-1)

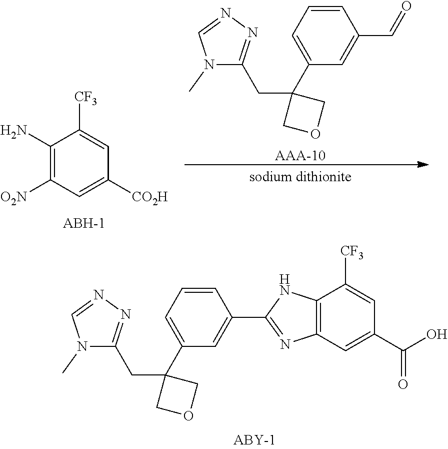

To a stirred solution of intermediate (AAA-10) (50 mg, 1 Eq, 200 μmol) and intermediate (ABH-1) (49 mg, 1 Eq, 200 μmol) in EtOH (1 mL) were added sodium hydrosulfite (102 mg, 3 Eq, 580 mmol) at 80° C. under nitrogen atmosphere. The residue was purified by Prep-TLC with DCM/MeOH (15/1). The crude product was purified by Prep-HPLC with the following conditions (Column: YMC-Actus Triart C18, 20*250 MM, 5 μm, 12 nm; Mobile Phase A: Water (0.1% NH₄HCO₃+0.10% NH₃·H₂O), Mobile Phase B: MeCN; Flow rate: 60 mL/min; Gradient: 2 B to 18 B in 9 min; Detector, UV 254/220 nm; RT: 8.55) to afford the title compound (ABY-1) (24.1 mg, 53 μmol, 26%) as a white solid. m/z 458.1 (M+H)⁺ (ES+). ¹H NMR (400 MHz, DMSO-d6) δ 8.39 (s, 1H), 8.21-8.09 (m, 3H), 7.94 (t, J=1.7 Hz, 1H), 7.51 (t, J=7.8 Hz, 1H), 7.16-7.11 (m, 1H), 5.01-4.97 (m, 4H), 3.58 (s, 2H), 2.98 (s, 3H).

### Example 131: Synthesis of N-(1-Methyl-1H-imidazol-2-yl)-2-(3-(3-((4-methyl-4H-1,2,4-triazol-3-yl)methyl)oxetan-3-yl)phenyl)-7-(trifluoromethyl)-1H-benzo[d]imidazole-5-carboxamide (ABZ-2)

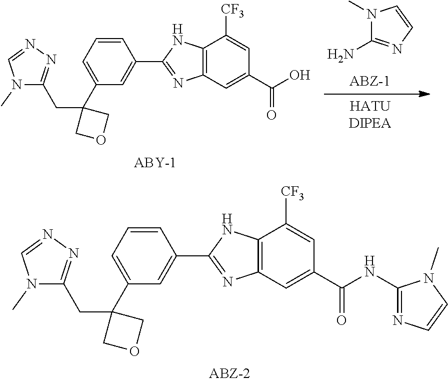

To a stirred mixture of intermediate (ABY-1) (50 mg, 1 Eq, 100 μmol) in DMF (5 mL) were added DIPEA (85 mg, 6 Eq, 650 μmol), HATU (83 mg, 2 Eq, 210 μmol) and 1-methylimidazol-2-amine (ABZ-1) (16 mg, 1.5 Eq, 160 μmol) at rt. The resulting mixture was stirred for 2 h at rt. The crude product was purified by reverse flash column chromatography with the following conditions: Column, C18; mobile phase, Water (0.1% NH₄HCO₃) and MeCN (0% ACN up to 50% in 10 min); Detector, UV 254/220 nm. The product-containing fractions were combined and concentrated in vacuo. The crude product was purified by Prep-HPLC with the following conditions: Column: SunFire Prep C18 OBD Column, 19×150 mm, 5 μm; Mobile Phase A: Water (0.1% NH₄HCO₃+0.1% NH₃·H₂O), Mobile Phase B: MeCN; Flow rate: 60 mL/min; Gradient: 30 B to 60 B in 7 min; Detector, UV 254/210 nm; RT: 6.08. This resulted in the title compound (ABZ-2) (4.2 mg, 7.8 μmol, 7.2%) as a white solid. m/z 537.4 (M+H)⁺ (ES+). ¹H NMR (400 MHz, Methanol-d4) δ 8.60 (d, J=1.4 Hz, 1H), 8.37 (s, 1H), 8.27 (s, 1H), 8.18-8.13 (d, J=8.0 Hz, 1H), 7.88 (s, 1H), 7.56 (t, J=7.8 Hz, 1H), 7.34-7.25 (m, 2H), 7.16 (d, J=7.6 Hz, 1H), 5.20-5.12 (m, 4H), 3.85 (s, 3H), 3.74 (s, 2H), 3.01 (s, 3H).

### Example 132: Synthesis of (R)-2-(3-(1-(4-methyl-4H-1,2,4-triazol-3-yl)propan-2-yl)phenyl)-5-(trifluoromethyl)-[1,2,4]triazolo[1,5-a]pyridine (ACA-2)

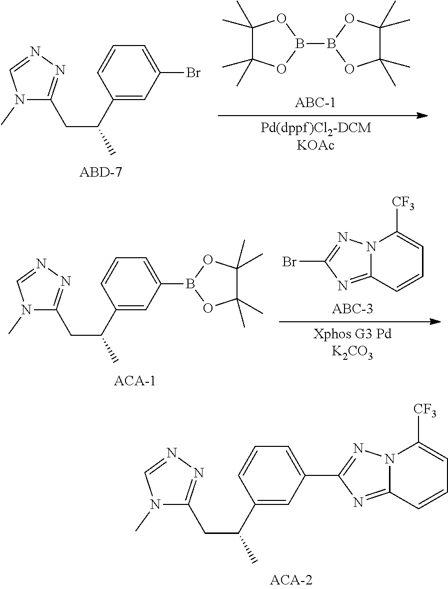

Step 1: (R)-4-Methyl-3-(2-(3-(4,4,5,5-tetramethyl-1,3,2-dioxaborolan-2-yl)phenyl)propyl)-4H-1,2,4-triazole (ACA-1). A solution of intermediate (ABD-7) (250 mg, 1 Eq, 892 μmol), bis(pinacolato)diboron (ABC-1) (453 mg, 2 Eq, 1.78 mmol) and KOAc (175 mg, 2 Eq, 1.78 mmol) in dioxane (3 mL) was added Pd(dppf)Cl₂-DCM (39 mg, 0.06 Eq, 54 μmol) at rt under nitrogen atmosphere. The mixture was stirred at 90° C. for 2 hours under nitrogen atmosphere. The mixture was cooled to rt and concentrated in vacuo. The residue was purified by Prep-TLC with DCM/MeOH (15/1) to afford the sub-title compound (ACA-1) (50 mg, 153 μmol, 17%) as a white solid. m/z 328.2 (M+H)⁺ (ES+).

### Step 2: (R)-2-(3-(1-(4-Methyl-4H-1,2,4-triazol-3-yl)propan-2-yl)phenyl)-5-(trifluoromethyl)[1,2,4]triazolo[1,5-a]pyridine (ACA-2)

A solution of the product from step 1 above (ACA-1) (50 mg, 1 Eq, 153 μmol), intermediate (ABC-3) (41 mg, 1 Eq, 153 μmol) and a solution of K₂CO₃ (63 mg, 3 Eq, 458 μmol) in dioxane (2 mL) and water (0.2 mL) was added XPhos Pd G₃ (10 mg, 0.08 Eq, 12 μmol) at rt under nitrogen atmosphere. The mixture was stirred at 100° C. for 1 hour under nitrogen atmosphere. The mixture was cooled to rt and concentrated in vacuo. The crude product was purified by Prep-HPLC with the following conditions: to afford the title compound (ACA-2) (3.8 mg, 9.8 μmol, 6.4%) as a white solid. m/z 387.1 (M+H)⁺ (ES+). ¹H NMR (400 MHz, Methanol-d4) δ 8.27 (s, 1H), 8.19-8.11 (m, 2H), 8.11-8.03 (m, 1H), 7.89-7.76 (m, 1H), 7.76-7.67 (m, 1H), 7.54-7.43 (m, 1H), 7.43-7.34 (m, 1H), 3.49-3.43 (m, 4H), 3.33-3.07 (m, 2H), 1.49 (d, J=1.9 Hz, 3H).

### Example 133: Synthesis of (R)-1-((2-(3-(3-((4-Methyl-4H-1,2,4-triazol-3-yl)methyl) oxetan-3-yl)phenyl)-7-(trifluoromethyl)-1H-benzo[d]imidazol-5-yl)methyl)pyrrolidin-3-ol (ACB-1)

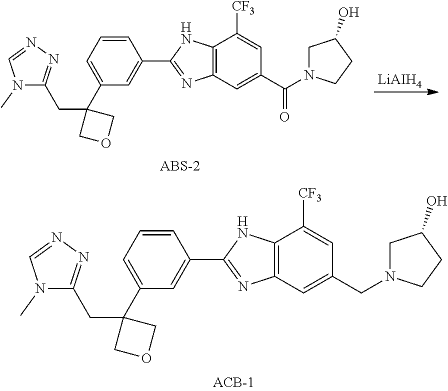

To a stirred solution of intermediate (ABS-2) (50 mg, 1 Eq, 95 μmol) in THF (8 mL) was added a solution of LiAlH₄ in THF (280 μl, 1 molar, 3 Eq, 285 μmol) at 0° C. under nitrogen atmosphere. The resulting mixture was stirred for 3 h at rt. The reaction was quenched by the addition of sodium sulfate decahydrate and diluted with water (50 mL) at 0° C. The resulting mixture was diluted with water and extracted with EtOAc (3×20 mL). The combined organic layers were washed with brine (2×20 mL), dried (Na₂SO₄). After filtration, the filtrate was concentrated in vacuo. The crude product was purified by Prep-HPLC with the following conditions: Column: YMC-Actus Triart C18 ExRS, 30*250 mm, 5 μm; mobile phase, Water (0.1% NH₄HCO₃+0.1% NH₃·H₂O) and MeCN (10% MeCN up to 40% in 7 min); UV detection at 254/220 nm. The product-containing fractions were combined and evaporated partially in vacuo and lyophilized overnight to afford the title compound (ACB-1) (5.4 mg, 11 μmol, 11%) as a white solid. m/z 513.2 (M+H)⁺ (ES+). ¹H NMR (400 MHz, Methanol-d4) δ 8.16 (s, 1H), 8.07 (d, J=7.8 Hz, 1H), 7.79 (s, 2H), 7.61 (s, 1H), 7.49 (t, J=7.8 Hz, 1H), 7.06-6.99 (m, 1H), 5.20-5.09 (m, 4H), 4.42-4.33 (m, 1H), 3.93-3.78 (m, 2H), 3.69 (s, 2H), 2.91 (s, 3H), 2.89-2.77 (m, 2H), 2.67-2.53 (m, 2H), 2.25-2.11 (m, 1H), 1.83-1.70 (m, 1H).

### Example 134: Synthesis of 1-Methyl-N-((2-(3-(3-((4-methyl-4H-1,2,4-triazol-3-yl)meth yl)oxetan-3-yl)phenyl)-7-(trifluoromethyl)-1H-benzo[d]imidazol-5-yl)methyl)-1H-imidazol-2-amine (ACC-1)

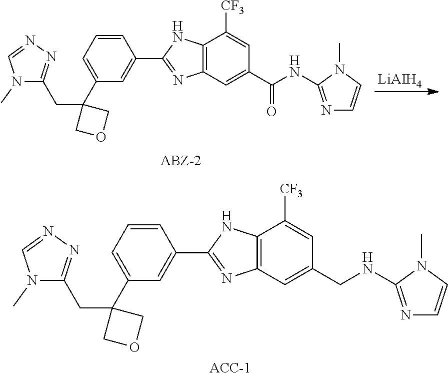

To a stirred solution of intermediate (ABZ-2) (60 mg, 1 Eq, 110 mmol)win added a solution of LiAlH₄ in THF (56 μl, 1 molar, 5 Eq, 560 μmol) at 0° C. under nitrogen atmosphere. The resulting mixture was stirred for 3 h at rt. The reaction was quenched by the addition of sodium sulfate decahydrate and diluted with water (50 mL) at 0° C. The resulting mixture was diluted with water and extracted with EtOAc (3×20 mL). The combined organic layers were washed with brine (2×20 mL), dried (Na₂SO₄). After filtration, the filtrate was concentrated in vacuo. The crude product was purified by reverse flash column chromatography with the following conditions: Column, C18; mobile phase, Water (0.1% NH₄HCO₃) and MeCN (10% MeCN up to 80% in 20 min); Detector, UV 254/220 nm and Prep-HPLC with the following conditions: (Column: YMC-Actus Triart C18 ExRS, 30*250 mm, 5 μm; Mobile Phase A: Water (0.1% NH₄HCO₃+0.1% NH₃·H₂O), Mobile Phase B: MeCN; Flow rate: 60 mL/min; Gradient: 13% B to 43% B in 7 min; Wave Length: 254/220 nm; RT: 6.32). The product-containing fractions were combined and evaporated partially in vacuo and lyophilized overnight to afford the title compound (ACC-1) (2.9 mg, 5.5 μmol, 4.4%) as a white solid. m/z 523.2 (M+H)⁺ (ES+). ¹H NMR (400 MHz, Methanol-d4) δ 8.15 (s, 1H), 8.05 (d, J=7.9 Hz, 1H), 7.89-7.73 (m, 2H), 7.62 (s, 1H), 7.48 (t, J=7.8 Hz, 1H), 7.03-6.98 (m, 1H), 6.61 (d, J=1.8 Hz, 1H), 6.54 (d, J=1.7 Hz, 1H), 5.16-5.08 (m, 4H), 4.62 (s, 2H), 3.68 (s, 2H), 3.44 (s, 3H), 2.90 (s, 3H).

### Example 135: Synthesis of 2-(2′-(4-Methyl-4H-1,2,4-triazol-3-yl)-[1,1′-biphenyl]-3-yl)-7-(trifluoromethyl)-1H-benzo[d]imidazole-5-carboxylic Acid (ACD-1)

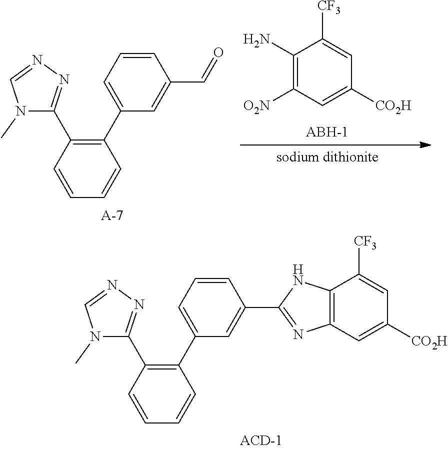

To a stirred mixture of intermediate (A-7) (32 mg, 1 Eq, 120 μmol) and intermediate (ABH-1) (30 mg, 1 Eq, 120 μmol) in EtOH (1 mL) and water (1 mL) was added sodium hydrosulfite (212 mg, 10 Eq, 1.22 mmol) at rt. The resulting mixture was stirred for overnight at 80° C. The mixture was cooled to rt and concentrated in vacuo. The crude product was purified by reverse flash column chromatography with the following conditions: Column, C18; mobile phase, Water (0.1% NH₄HCO₃) and MeCN (30% MeCN up to 70% in 25 min); Detector, UV 254/220 nm. The resulting mixture was concentrated in vacuo. The crude product was purified by Prep-HPLC with the following conditions (Column: XBridge Prep OBD C18 Column, 30×150 mm 5 μm; Mobile Phase A: Water (0.1% NH₄HCO₃+0.1% NH₃·H₂O), Mobile Phase B: MeCN; Flow rate: 60 mL/min; Gradient: 10% B to 30% B in 10 min; Wave Length: 254/220 nm; RT: 9.27) to afford the title compound (ACD-1) (5.7 mg, 12 μmol, 10%) as a white solid. m/z 464.2 (M+H)⁺ (ES+). ¹H NMR (400 MHz, Methanol-d4) δ 8.46 (s, 1H), 8.37 (s, 1H), 8.24 (d, J=15.4 Hz, 2H), 8.16 (d, J=7.8 Hz, 1H), 7.85-7.76 (m, 2H), 7.72-7.63 (m, 2H), 7.52 (t, J=7.8 Hz, 1H), 7.28 (d, J=7.7 Hz, 1H), 3.21 (s, 3H).

### Example 136: Synthesis of (2-(2′-(4-Methyl-4H-1,2,4-triazol-3-yl)-[1,1′-biphenyl]-3-yl)-7-(trifluoromethyl)-1H-benzo[d]imidazol-5-yl)(5-azaspiro[2.4]heptan-5-yl)methanone (ACE-1)

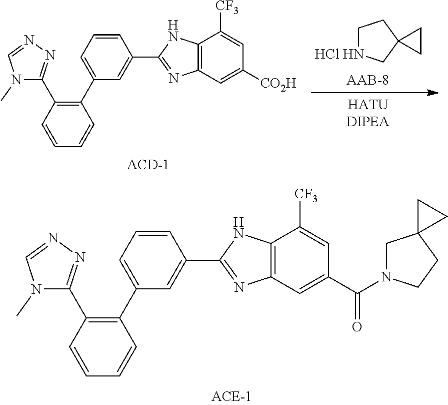

To a stirred mixture of compound (ACD-1) (20 mg, 1 Eq, 40 μmol) and intermediate (AAB-8) (4.1 mg, 1 Eq, 40 mmol) in DMF (1.5 mL) were added HATU (16 mg, 1 Eq, 40 μmol) and DIPEA (17 mg, 3 Eq, 120 μmol) at rt. The resulting mixture was stirred for 2 h at rt. The crude product was purified by reverse flash column chromatography with the following conditions: Column, C18; mobile phase, Water (0.1% NH4HCO3) and MeCN (30% MeCN up to 70% in 30 min); Detector, UV 254/220 nm. The crude product was purified by Prep-HPLC with the following conditions (Column: XBridge Prep OBD C18 Column, 30×150 mm 5 um; Mobile Phase A: Water (0.1% NH₄HCO₃+0.1% NH₃·H₂O), Mobile Phase B: MeCN; Flow rate: 60 mL/min; Gradient: 25% B to 50% B in 10 min; Wave Length: 254/220 nm; RT: 9.67) to afford the title compound (ACE-1) (5 mg, 9.2 μmol, 21%) as a white solid. m/z 543.3 (M+H)⁺ (ES+). ¹H NMR (400 MHz, Methanol-d4) δ 8.37 (s, 1H), 8.18 (d, J=21.1 Hz, 2H), 8.05 (d, J=15.7 Hz, 1H), 7.79 (d, J=11.9 Hz, 3H), 7.67 (t, J=2.9 Hz, 2H), 7.52 (t, J=7.8 Hz, 1H), 7.29 (d, J=7.7 Hz, 1H), 3.89-3.72 (m, 2H), 3.53 (d, J=38.1 Hz, 2H), 3.22 (s, 3H), 1.98 (t, J=7.1 Hz, 1H), 1.90 (t, J=6.6 Hz, 1H), 0.74 (d, J=15.1 Hz, 2H), 0.62 (d, J=16.0 Hz, 2H).

### Example 137: Synthesis of 5-((5-Azaspiro[2.4]heptan-5-yl)methyl)-2-(2′-(4-methyl-4H-1,2,4-triazol-3-yl)-[1,1′-biphenyl]-3-yl)-7-(trifluoromethyl)-1H-benzo[d]imidazole (ACF-1)

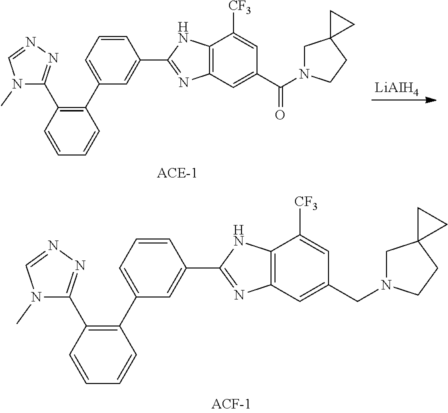

To a stirred mixture of intermediate (ACE-1) (53 mg, 1 Eq, 90 μmol) in THF (1.5 mL) was added LiAlH₄ (7.4 mg, 2 Eq, 190 μmol) at 0° C. The resulting mixture was stirred for 2 h at it. The reaction was then quenched by the addition of 1 mL of ice water at 0° C. The resulting mixture was diluted with water and extracted with DCM (3×20 mL). The combined organic layers were washed with brine (2×20 mL), dried (Na₂SO₄). After filtration, the filtrate was concentrated in vacuo. The crude product was purified by reverse flash column chromatography with the following conditions: Column, C18; mobile phase, Water (0.1% NH₄HCO₃) and MeCN (20% MeCN up to 50% in 20 min); Detector, UV 254/220 nm. The crude product was purified by Prep-HPLC with the following conditions (Column: YMC-Actus Triart C18 ExRS, 30*250 mm, 5 μm; Mobile Phase A: Water (0.1% NH₄HCO₃+0.1% NH₃·H₂O), Mobile Phase B: MeCN; Flow rate: 60 mL/min; Gradient: 5% B to 50% B in 2 min, 50% B to 70% B in 10 min; Wave Length: 254/220 nm; RT: 7.53) to afford the title compound (ACF-1) (15.9 mg, 30 μmol, 30%) as a white solid. m/z 529.2 (M+H)⁺ (ES+). ¹H NMR (400 MHz, Methanol-d4) δ 8.37 (s, 1H), 8.21-8.08 (m, 2H), 7.93-7.72 (m, 3H), 7.71-7.59 (m, 3H), 7.50 (t, J=7.8 Hz, 1H), 7.26 (d, J=7.8 Hz, 1H), 3.95-3.83 (m, 2H), 3.20 (s, 3H), 2.98-2.78 (m, 2H), 2.74-2.48 (m, 2H), 1.91 (s, 2H), 0.59 (d, J=9.9 Hz, 4H).

### Example 138; Synthesis of 1-(2-Hydroxyethyl)-3-(3-(3-((4-methyl-4H-1,2,4-triazol-3-yl)methyl)oxetan-3-yl)phenyl)-5-(trifluoromethyl)pyridin-2(1H)-one (ACG-3)

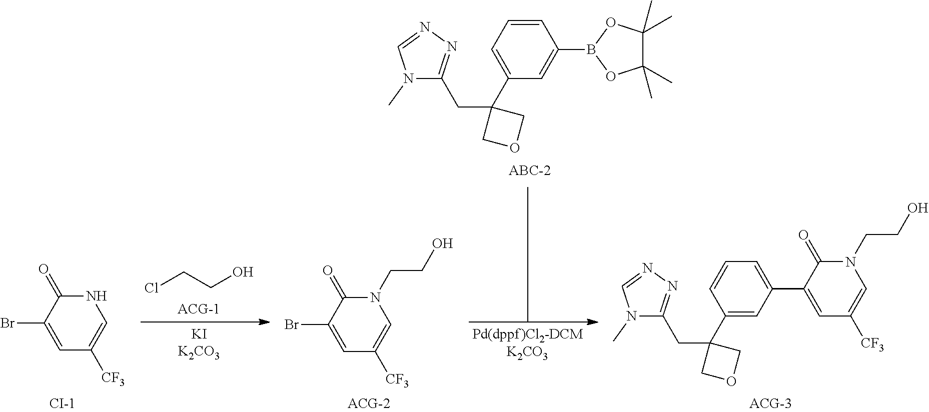

Step 1: 3-Bromo-1-(2-hydroxyethyl)-5-(trifluoromethyl)-pyridin-2-one (ACG-2). To a stirred solution of intermediate (CI-1) (400 mg, 1 Eq, 1.65 mmol) and 2-bromoethanol (ACG-1) (248 mg, 1.2 Eq, 1.98 mmol) in acetone (8 mL) were added K₂CO₃ (571 mg, 2.5 Eq, 4.13 mmol) and KI (137 mg, 0.5 Eq, 820 μmol) at rt. The resulting mixture was stirred for 16 hours at 50° C. The mixture was cooled to rt and concentrated in vacuo. The crude product was purified by reverse flash column chromatography with the following conditions: Column, C18; mobile phase, Water (0.1% NH₄HCO₃) and MeCN (20% MeCN up to 40% in 10 min); Detector, UV 254/220 nm. This resulted in the sub-title compound (ACG-2) (317 mg, 1.11 mmol, 67%) as a white solid. m/z 286.0/288.0 (M+H)⁺ (ES+).

Step 2: 1-(2-Hydroxyethyl)-3-(3-[3-[(4-methyl-1,2,4-triazol-3-yl)-methyl]-oxetan-3-yl]-phenyl)-5-(trifluoromethyl)-pyridin-2-one (ACG-3). To a stirred mixture of the product from step 1 above (ACG-2) (86 mg, 1 Eq, 300 μmol), intermediate (ABC-2) (117 mg, 1.1 Eq, 330 mmol) and K₂CO₃ (124 mg, 3 Eq, 900 mmol) in 1,4-dioxane (4 mL) and water (1 mL) were added Pd(dppf)Cl₂-DCM (44 mg, 0.2 Eq, 60 mmol) at rt under nitrogen atmosphere. The resulting mixture was stirred for 2 hours at 80° C. under nitrogen atmosphere. The mixture was cooled to rt and concentrated in vacuo. The crude product was purified by reverse flash column chromatography with the following conditions: Column, C18; mobile phase, Water (0.1% FA) and MeCN (25% MeCN up to 45% in 8 min); Detector, UV 254/220 nm. The product-containing fractions were combined and concentrated in vacuo. The crude product was purified by Prep-HPLC with the following conditions: Column: SunFire Prep C18 OBD Column, 30×150 mm, 5 μm; Mobile Phase A: Water (0.1% FA), Mobile Phase B: MeCN; Flow rate: 60 mL/min; Gradient: 10 B to 40 B in 7 min; Detector, UV 254/210 nm; RT: 6.32. The product-containing fractions were combined and evaporated partially in vacuo and lyophilized overnight to afford the title compound (ACG-3) (15.4 mg, 35 μmol, 11%) as a white solid. m/z 435.3 (M+H)⁺ (ES+). ¹H NMR (400 MHz, Methanol-d4) δ 8.19 (s, 1H), 8.16-8.10 (m, 1H), 7.66 (d, J=2.7 Hz, 1H), 7.56-7.48 (m, 1H), 7.38 (t, J=7.7 Hz, 1H), 7.13 (t, J=1.8 Hz, 1H), 6.89-6.82 (m, 1H), 5.10-5.04 (m, 4H), 4.24-4.17 (m, 2H), 3.91-3.84 (m, 2H), 3.66 (s, 2H), 2.85 (s, 3H).

### Example 139: Synthesis of 1-(2-(3-Hydroxyazetidin-1-yl)ethyl)-3-(3-(3-((4-methyl-4H-1,2,4-triazol-3-yl)methyl)oxetan-3-yl)phenyl)-5-(trifluoromethyl)pyridin-2(1H)-one (ACH-5)

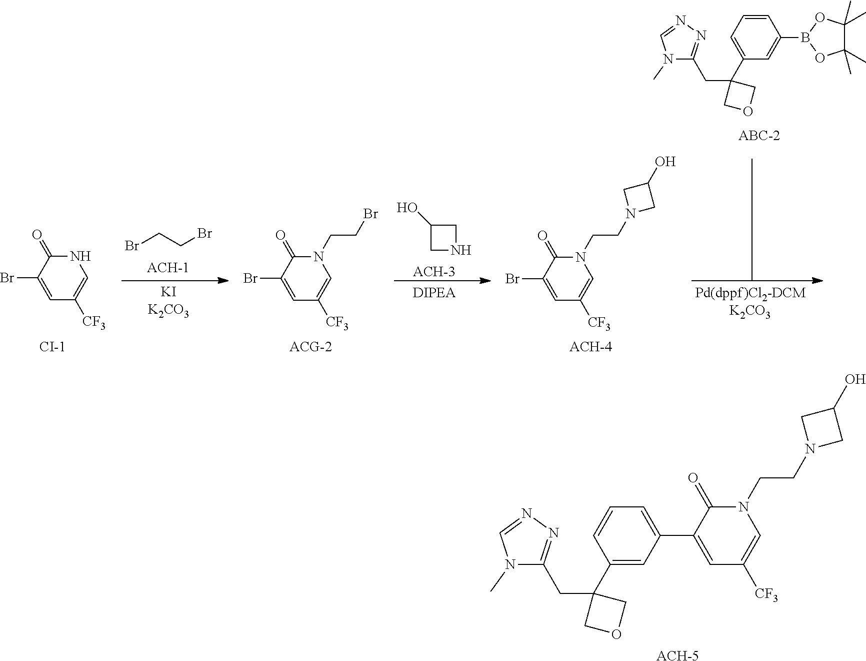

Step 1: 3-Bromo-1-(2-bromoethyl)-5-(trifluoromethyl)pyridin-2(1H)-one (ACH-2). To a stirred solution of 3-bromo-5-(trifluoromethyl) pyridin-2-ol (CI-1) (250 mg, 1 Eq, 1.03 mmol) and K₂CO₃ (428 mg, 3.10 mmol, 3 Eq) in MeCN (5 mL) were added 1-bromo-2-chloroethane (296 mg, 2 Eq, 2.07 mmol) at 50° C. under nitrogen atmosphere. The resulting mixture was stirred for overnight at 50° C. under nitrogen atmosphere. The resulting mixture was cooled to rt and concentrated in vacuo. The crude product was purified by reverse flash column chromatography with the following conditions: Column, C18; mobile phase, Water (0.1% NH₄HCO₃) and MeCN (10% MeCN up to 50% in 20 min); Detector, UV 254/220 nm. The resulting mixture was concentrated in vacuo. This resulted in the sub-title compound (ACH-2) (240 mg, 688 μmol, 67%) as a light brown solid. m/z 349.9/351.9 (M+H)⁺ (ES+).

Step 2: 3-Bromo-1-(2-(3-hydroxyazetidin-1-yl)ethyl)-5-(trifluoromethyl)pyridin-2(1H)-one (ACH-4). To a stirred solution of intermediate (ACH-2) (180 mg, 1 Eq, 600 μmol) and DIPEA (229 mg, 3 Eq, 1.8 mmol) in NMP (4 mL) were added azetidin-3-ol (ACH-3) (86 mg, 2 Eq, 1.2 mmol) at 80° C. under nitrogen atmosphere. The resulting mixture was stirred for 4 hours at 80° C. under nitrogen atmosphere. The mixture was cooled to rt and concentrated in vacuo. The crude product was purified by reverse flash column chromatography with the following conditions: Column, C18; mobile phase, Water (0.1% NH₄HCO₃) and MeCN (10% MeCN up to 50% in 10 min); Detector, UV 254/220 nm. This resulted in the sub-title compound (ACH-4) (183 mg, 538 μmol, 91%) as a light yellow solid. m/z 341.0/343.0 (M+H)⁺ (ES+).

Step 3: 1-(2-(3-Hydroxyazetidin-1-yl)ethyl)-3-(3-(3-((4-methyl-4H-1,2,4-triazol-3-yl)methyl) oxetan-3-yl)phenyl)-5-(trifluoromethyl) pyridin-2(1H)-one (ACH-5). To a solution of the product from step 1 above (ACH-4) (65 mg, 1 Eq, 190 mmol), intermediate (ABC-2) (135 mg, 2 Eq, 380 mmol) and K₂CO₃ (79 mg, 3 Eq, 570 μmol) in 1,4-dioxane (4 mL) and water (2 mL) was added Pd(dppf)Cl₂-DCM (14 mg, 0.1 Eq, 20 μmol) at rt under nitrogen atmosphere. After stirring for overnight at 80° C. under nitrogen atmosphere, the resulting mixture was cooled to rt and concentrated in vacuo. The crude product was purified by Prep-HPLC with the following conditions (Column: XBridge Shield RP18 OBD Column, 30*150 mm, 5 μm; Mobile Phase A: Water (0.1% NH₄HCO₃+0.1% NH₃·H₂O), Mobile Phase B: MeCN; Flow rate: 60 mL/min; Gradient: 10 B to 40 B in 7 min; Detector, UV 254/220 nm; RT: 6.1) to afford the title compound (ACH-5) (10.3 mg, 21 μmol, 11%) as a white solid. m/z 490.1 (M+H)⁺ (ES+). ¹H NMR (400 MHz, Methanol-d4) δ 8.20 (s, 1H), 8.17-8.13 (m, 1H), 7.64 (d, J=2.6 Hz, 1H), 7.54-7.49 (m, 1H), 7.38 (t, J=7.7 Hz, 1H), 7.12 (t, J=1.8 Hz, 1H), 6.89-6.84 (m, 1H), 5.11-5.03 (m, 4H), 4.40-4.31 (m, 1H), 4.09 (t, J=6.2 Hz, 2H), 3.71-3.63 (m, 4H), 3.00-2.94 (m, 2H), 2.92-2.83. (m, 5H).

### Example 140: Synthesis of 1-(2-(3-Hydroxyazetidin-1-yl)ethyl)-3-(2′-(4-methyl-4H-1,2,4-triazol-3-yl)-[1,1′-biphenyl]-3-yl)-5-(trifluoromethyl)pyridin-2(1H)-one (ACI-3)

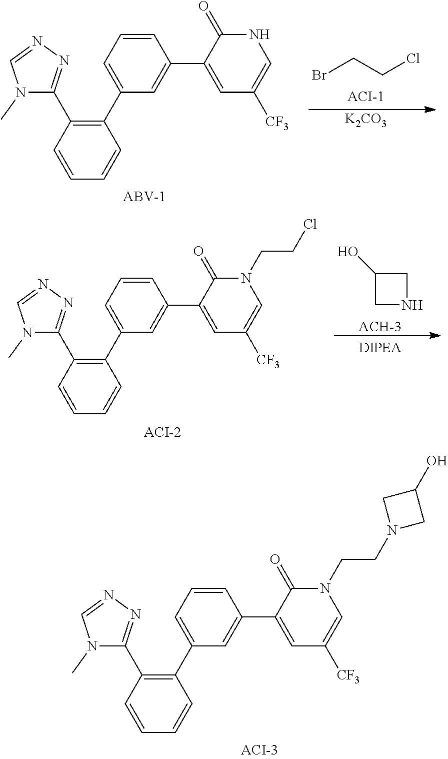

Step 1: 1-(2-Chloroethyl)-3-(2′-(4-methyl-4H-1,2,4-triazol-3-yl)-[1,1′-biphenyl]-3-yl)-5-(trifluoromethyl) pyridin-2(1H)-one (ACI-2). To a stirred solution of intermediate (ABV-1) (250 mg, 1 Eq, 631 μmol) and K₂CO₃ (261 mg, 3 Eq, 1.89 mmol) in MeCN (5 mL) were added 1-bromo-2-chloroethane (ACI-1) (179 mg, 2 Eq, 1.26 mmol) at rt under nitrogen atmosphere. The resulting mixture was stirred for 1.5 hours at 60° C. under nitrogen atmosphere. The mixture was cooled to rt and concentrated in vacuo. The crude product was purified by reverse flash column chromatography with the following conditions: Column, C18; mobile phase, Water (0.1% NH₄HCO₃) and MeCN (10% MeCN up to 50% in 20 min); Detector, UV 254/220 nm. The resulting mixture was concentrated in vacuo. This resulted in the sub-title compound (ACI-2) (260 mg, 568 μmol, 90%) as a light brown solid. m/z 459.1/461.1 (M+H)⁺ (ES+).

Step 2: 1-(2-(3-Hydroxyazetidin-1-yl)ethyl)-3-(2′-(4-methyl-4H-1,2,4-triazol-3-yl)-[1,1′-biphenyl]-3-yl)-5-(trifluoromethyl)pyridin-2(1H)-one (ACI-3). To a solution of the product from step 1 above (ACI-2) (60 mg, 1 Eq, 130 mmol) and azetidin-3-ol (ACH-3) (48 mg, 5 Eq, 650 μmol) in NMP (2 mL) was added DIPEA (85 mg, 5 Eq, 650 μmol). After stirring for 4 h at 50° C. under nitrogen atmosphere, the mixture was cooled to rt and concentrated in vacuo. The crude product was purified by Prep-HPLC with the following conditions (Column: XBridge Shield RP18 OBD Column, 30*150 mm, 5 μm; Mobile Phase A: Water (0.1% NH₄HCO₃+0.1% NH₃·H₂O), Mobile Phase B: MeCN; Flow rate: 60 mL/min; Gradient: 10 B to 40 B in 7 min; Detector, UV 254/220 nm; RT: 6.1) to afford the title compound (ACI-3) (3 mg, 6.0 μmol, 4.6%) as an off-white solid. m/z 496.1 (M+H)⁺ (ES+). ¹H NMR (400 MHz, Methanol-d4) δ 8.38 (s, 1H), 8.16-8.13 (m, 1H), 7.79-7.69 (m, 2H), 7.64-7.59 (m, 4H), 7.49 (t, J=1.8 Hz, 1H), 7.42 (t, J=7.7 Hz, 1H), 7.28-7.21 (m, 1H), 4.39-4.30 (m, 1H), 4.10 (t, J=6.2 Hz, 2H), 3.70-3.63 (m, 2H), 3.12 (s, 3H), 3.00-2.94 (m, 2H), 2.89 (t, J=6.2 Hz, 2H).

### Example 141: Synthesis of 2-(3-(3-((4-Methyl-4H-1,2,4-triazol-3-yl)methyl)oxetan-3-yl) phenyl)-5-(2-(pyrrolidin-1-yl)ethoxy)-7-(trifluoromethyl)-1H-benzo[d]imidazole (ACJ-5)

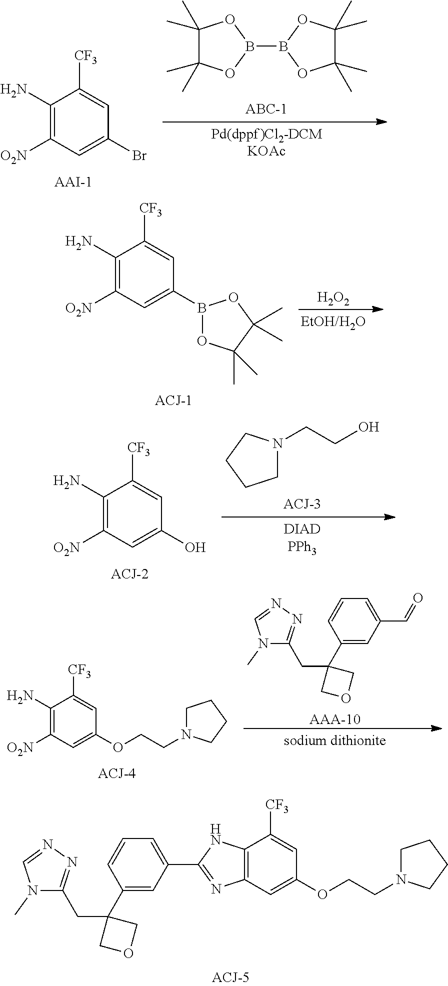

Step 1: 2-Nitro-4-(4,4,5,5-tetramethyl-1,3,2-dioxaborolan-2-yl)-6-(trifluoromethyl)aniline (ACJ-1). To a stirred solution of intermediate (AAI-1) (285 mg, 1 Eq, 1.00 mmol) and bis(pinacolato)diboron (ABC-1) (381 mg, 1.5 Eq, 1.50 mmol) and KOAc (294 mg, 3 Eq, 3.00 mmol) in dioxane (5 mL) was added Pd(dppf)Cl₂-DCM (73 mg, 0.1 Eq, 100 μmol) at rt under nitrogen atmosphere. The resulting mixture was stirred for overnight at 100° C. under nitrogen atmosphere. The mixture was cooled to rt and concentrated in vacuo. The resulting mixture was diluted with water and extracted with EtOAc (3×50 mL). The combined organic layers were washed with brine (2×50 mL), dried (Na₂SO₄). After filtration, the filtrate was concentrated in vacuo. This resulted in the sub-title compound (ACJ-1) (300 mg) as a white solid. m/z 333.1 (M+H)⁺ (ES+).

Step 2: 4-Amino-3-nitro-5-(trifluoromethyl)phenol (ACJ-2). To a stirred mixture of the product from step 1 above (ACJ-1) (500 mg, 1 Eq, 1.50 mmol) in AcOH (20 mL) were added hydrogen peroxide (1 mL, 98% Wt, 28.5 Eq, 42.9 mmol) and THF (3 mL) at rt. The resulting mixture was stirred overnight at rt. The mixture was concentrated in vacuo. The residue was purified by Prep-TLC with EtOAc/petroleum ether (1/10) to afford the sub-title compound (ACJ-2) (165 mg, 743 μmol, 49%) as a white solid. m/z 223.0 (M+H)⁺ (ES+).

Step 3: 2-Nitro-4-(2-(pyrrolidin-1-yl)ethoxy)-6-(trifluoromethyl)aniline (ACJ-4). To a stirred mixture of the product from step 2 above (ACJ-2) (145 mg, 1 Eq, 650 μmol) and epolamine (ACJ-3) (90 mg, 1.2 Eq, 784 μmol) in THF (10 mL) were added PPh₃ (257 mg, 1.5 Eq, 980 μmol) and DIAD (198 mg, 1.5 Eq, 980 μmol) at rt under nitrogen atmosphere. The final reaction mixture was irradiated with microwave radiation for 3 h at 70° C. The mixture was cooled to rt and concentrated in vacuo. The crude product was purified by reverse flash column chromatography with the following conditions: Column, C18; mobile phase, Water (0.1% NH₄HCO₃) and MeCN (36% MeCN up to 48% in 10 min); Detector, UV 254/220 nm. The product-containing fractions were combined and concentrated in vacuo. This resulted in the sub-title compound (ACJ-4) (160 mg, 501 μmol, 77%) as a white solid. m/z 320.1 (M+H)⁺ (ES+).

Step 4: 2-(3-(3-((4-Methyl-4H-1,2,4-triazol-3-yl)methyl)oxetan-3-yl)phenyl)-5-(2-(pyrrolidin-1-yl)ethoxy)-7-(trifluoromethyl)-1H-benzo[d]imidazole (ACJ-5). To a stirred mixture of the product from step 3 above (ACJ-4) (50 mg, 1 Eq, 150 mmol) and intermediate (AAA-10) (40 mg, 1 Eq, 150 μmol) in EtOH (2 mL) were added sodium hydrosulfide (136 mg, 5 Eq, 780 mmol) at rt. The resulting mixture was stirred for 3 h at 60° C. The mixture was cooled to rt and concentrated in vacuo. The crude product was purified by reverse flash column chromatography with the following conditions: Column, C18; mobile phase, Water (0.1% NH₄HCO₃) and MeCN (20% MeCN up to 80% in 20 min); Detector, UV 254/220 nm. The product-containing fractions were combined and concentrated in vacuo. The crude product was purified by Prep-HPLC with the following conditions: Column: SunFire Prep C18 OBD Column, 19×150 mm, 5 μm; Mobile Phase A: Water (0.1% NH₄HCO₃+0.1% NH₃·H₂O), Mobile Phase B: MeCN; Flow rate: 60 mL/min; Gradient: 30 B to 60 B in 7 min; Detector, UV 254/210 nm; RT: 6.08. This resulted in the title compound (ACJ-5) (9.5 mg, 18 μmol, 11%) as a white solid. m/z 527.2 (M+H)⁺ (ES+). ¹H NMR (400 MHz, Methanol-d4) δ 8.48 (s, 1H), 8.18 (s, 1H), 8.07 (d, J=7.8 Hz, 1H), 7.77 (s, 1H), 7.55-7.42 (m, 2H), 7.36-7.29 (m, 1H), 7.08 (d, J=7.7 Hz, 1H), 5.15 (d, J=1.5 Hz, 4H), 4.46 (t, J=4.9 Hz, 2H), 3.69 (d, J=11.9 Hz, 4H), 3.47 (s, 4H), 2.95 (s, 3H), 2.18-2.08 (m, 4H).

### Example 142: Synthesis of 5-(1-(5-Azaspiro[2.4]heptan-5-yl)ethyl)-2-(3-(3-((4-methyl-4H-1,2,4-triazol-3-yl)methyl)oxetan-3-yl)phenyl)-7-(trifluoromethyl)-1H-benzo[d]imidazole (ACK-3)

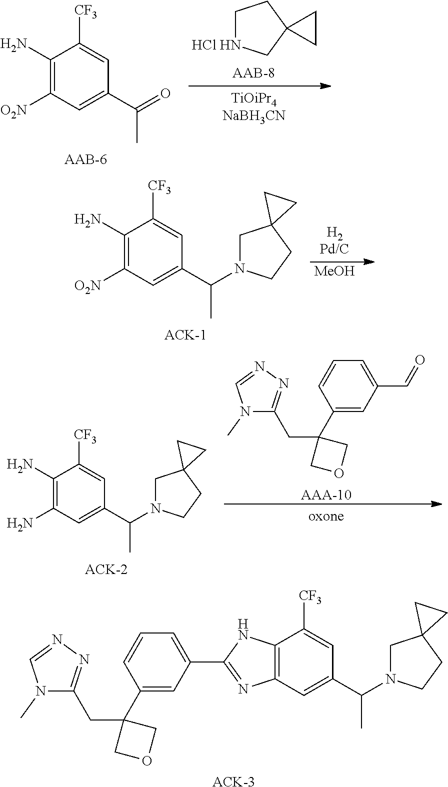

Step 1: 4-(1-(5-Azaspiro[2.4]heptan-5-yl)ethyl)-2-nitro-6-(trifluoromethyl)aniline (ACK-1). Into a 40 mL sealed tube were added intermediate (AAB-6) (200 mg, 1 Eq, 806 μmol), intermediate (AAB-8) (94 mg, 1.2 Eq, 967 μmol) and TiOiPr₄ (458 mg, 2 Eq, 1.61 mmol) in DCM (15 mL) at rt. To the above mixture was added NaBH₃CN (101 mg, 2 Eq, 1.61 mmol) over 5 min at rt. The resulting mixture was stirred for additional 2 h at 40° C. The mixture was allowed to cool down to rt. The resulting mixture was concentrated in vacuo. The crude product was purified by reverse flash column chromatography with the following conditions: Column, C18; mobile phase, Water (0.1% NH₄HCO₃) and MeCN (10% MeCN up to 60% in 11 min); Detector, UV 254/220 nm. The product-containing fractions were combined and concentrated in vacuo to afford the sub-title compound (ACK-1) (150 mg, 455 μmol, 57%) as yellow oil. m/z 330.1 (M+H)⁺ (ES+).

Step 2: 5-(1-(5-Azaspiro[2.4]heptan-5-yl)ethyl)-3-(trifluoromethyl)benzene-1,2-diamine (ACK-2). To a solution of the product from step 1 above (ACK-1) (150 mg, 1 Eq, 455 μmol) in MeOH (5 mL) was added Pd/C (10% Wt, 100 mg) under nitrogen atmosphere in a 50 mL round-bottom flask. The mixture was hydrogenated at rt for 2 h under hydrogen atmosphere using a hydrogen balloon, filtered and concentrated in vacuo. This resulted in the sub-title compound (ACK-2) (120 mg, 401 μmol, 88%) as a yellow oil. m/z 300.2 (M+H)⁺ (ES+).

Step 3: 5-(1-(5-Azaspiro[2.4]heptan-5-yl)ethyl)-2-(3-(3-((4-methyl-4H-1,2,4-triazol-3-yl) methyl)oxetan-3-yl)phenyl)-7-(trifluoromethyl)-1H-benzo[d]imidazole (ACK-3). Into a 20 mL sealed tube were added the product from step 2 above (ACK-2) (40 mg, 1 Eq, 134 μmol) and intermediate (AAA-10) (34 mg, 1 Eq, 134 μmol) in DMF (2 mL) and water (0.6 mL) at rt. To the above mixture was added Oxone (82 mg, 1 Eq, 134 μmol) over 3 min at rt. The resulting mixture was stirred for additional 3 h at rt. The mixture was concentrated in vacuo. The crude product was purified by Prep-HPLC with the following conditions: Column: SunFire Prep C18 OBD Column, 19×150 mm, 5 μm; Mobile Phase A: Water (0.1% NH₄HCO₃+0.1% NH₃·H₂O), Mobile Phase B: MeCN; Flow rate: 60 mL/min; Gradient: 30 B to 60 B in 7 min; Detector, UV 254/210 nm; RT: 6.08 to afford the title compound (ACK-3) (1.8 mg, 3.4 μmol, 2.5%) as a white solid. m/z 537.3 (M+H)⁺ (ES+). ¹H NMR (400 MHz, Methanol-d4) δ 8.18 (s, 1H), 8.10 (d, J=7.8 Hz, 1H), 7.84 (d, J=26.0 Hz, 2H), 7.67 (s, 1H), 7.52 (t, J=7.8 Hz, 1H), 7.06 (d, J=7.7 Hz, 1H), 5.20-5.08 (m, 4H), 3.71 (s, 2H), 3.63 (d, J=18.3 Hz, 1H), 2.93 (s, 4H), 2.73 (d, J=9.3 Hz, 2H), 2.47 (d, J=9.5 Hz, 1H), 1.89 (t, J=7.0 Hz, 2H), 1.53 (d, J=6.6 Hz, 3H), 1.37-1.23 (m, 1H), 0.60 (d, J=8.3 Hz, 4H).

### Example 143: Synthesis of(S)-1-((2-(3-(3-((4-Methyl-4H-1,2,4-triazol-3-yl)methyl) oxetan-3-yl)phenyl)-7-(trifluoromethyl)-1H-benzo[d]imidazol-5-yl) methyl)pyrrolidin-3-ol (ACL-1)

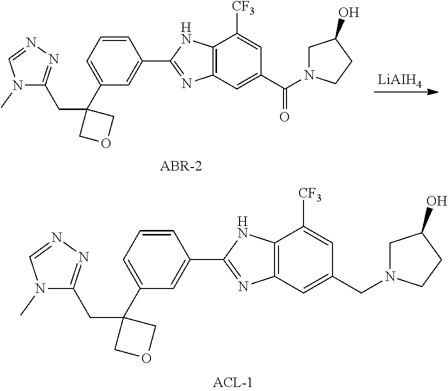

To a stirred solution of intermediate (ABR-2) (36 mg, 1 Eq, 68 μmol) in THF (6 mL) was added a solution of LiAlH₄ in THF (210 μl, 1 molar, 3 Eq, 200 μmol) dropwise at 0° C. under nitrogen atmosphere. The resulting mixture was stirred for overnight at rt under nitrogen atmosphere. The mixture was cooled to rt. The reaction was quenched by the addition of sodium sulfate decahydrate and diluted with water and extracted with EtOAc (3×10 mL). The combined organic layers were washed with brine (2×10 mL), dried (Na₂SO₄). After filtration, the filtrate was concentrated in vacuo. The crude product was purified by reverse flash column chromatography with the following conditions: Column, C18; mobile phase, Water (0.1% FA) and MeCN (10% MeCN up to 80% in 20 min); Detector, UV 254/220 nm and Prep-HPLC with the following conditions: Column: YMC-Actus Triart C18 ExRS, 30*250 mm, 5 μm; mobile phase, Water (0.1% NH₄HCO₃+0.1% NH₃·H₂O) and MeCN (10% MeCN up to 40% in 7 min); UV detection at 254/220 nm. The product-containing fractions were combined and evaporated partially in vacuo and lyophilized overnight to afford the title compound (ACL-1) (1.4 mg, 2.7 μmol, 3.8%) as a white solid. m/z 513.3 (M+H)⁺ (ES+). ¹H NMR (400 MHz, Methanol-d4) δ 8.18 (s, 1H), 8.11-8.07 (m, 1H), 7.86-7.79 (m, 2H), 7.62 (s, 1H), 7.51 (t, J=7.8 Hz, 1H), 7.06-7.01 (m, 1H), 5.23-5.07 (m, 4H), 4.44-4.34 (m, 1H), 3.91-3.77 (m, 2H), 3.71 (s, 2H), 2.93 (s, 3H), 2.89-2.76 (m, 2H), 2.63-2.51 (m, 2H), 2.25-2.13 (m, 1H), 1.80-1.71 (m, 1H).

### Example 144: Synthesis of (S)-5-(1-(5-Azaspiro[2.4]heptan-5-yl)ethyl)-2-(3-(3-((4-methyl-4H-1,2,4-triazol-3-yl)methyl)oxetan-3-yl)phenyl)-7-(trifluoromethyl)-1H-benzo[d]imidazole (ACM-1)

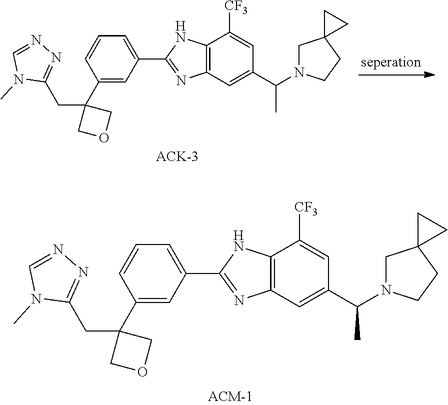 [SMILES: CC(c1cc(C(F)(F)F)c2[nH]c(-c3cccc(C4(Cc5nncn5C)COC4)c3)nc2c1)N1CCC2(CC2)C1 ; C[C@@H](c1cc(C(F)(F)F)c2[nH]c(-c3cccc(C4(Cc5nncn5C)COC4)c3)nc2c1)N1CCC2(CC2)C1]

The crude product intermediate (ACK-3) (60 mg, 1 Eq, 112 μmol) was separated by Chiral-Prep-HPLC with the following conditions: Column: CHIRALPAK IE, 2*25 cm, 5 μm; Mobile Phase A: MTBE (0.5% 2M NH₃-MeOH)-HPLC, Mobile Phase B: MeOH-HPLC; Flow rate: 20 mL/min; Gradient: 15% B to 15% B in 17 min; Wave Length: 220/254 nm; RTL 11.557; RT2: 14.464. This resulted in the title compound (ACM-1) (19.8 mg, 37 μmol, 32%) as a white solid. m/z 537.2 (M+H)⁺ (ES+). ¹H NMR (400 MHz, Methanol-d4) δ 8.18 (s, 1H), 8.09 (d, J=7.8 Hz, 1H), 7.91-7.77 (m, 2H), 7.66 (s, 1H), 7.51 (t, J=7.8 Hz, 1H), 7.07-7.02 (m, 1H), 5.21-5.10 (m, 4H), 3.71 (s, 2H), 3.60-3.45 (m, 1H), 2.93 (s, 4H), 2.72-2.57 (m, 2H), 2.42 (d, J=9.3 Hz, 1H), 1.88 (t, J=7.0 Hz, 2H), 1.51 (d, J=6.6 Hz, 3H), 0.64-0.51 (m, 4H).

### Example 145: Synthesis of (R)-5-(1-(5-Azaspiro[2.4]heptan-5-yl)ethyl)-2-(3-(3-((4-methyl-4H-1,2,4-triazol-3-yl)methyl)oxetan-3-yl)phenyl)-7-(trifluorome thyl)-1H-benzo[d]imidazole (ACN-1)

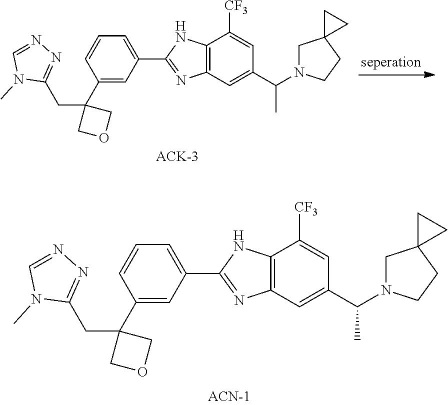 [SMILES: CC(c1cc(C(F)(F)F)c2[nH]c(-c3cccc(C4(Cc5nncn5C)COC4)c3)nc2c1)N1CCC2(CC2)C1 ; C[C@H](c1cc(C(F)(F)F)c2[nH]c(-c3cccc(C4(Cc5nncn5C)COC4)c3)nc2c1)N1CCC2(CC2)C1]

The crude product intermediate (ACK-3) (60 mg, 1 Eq, 112 μmol) was separated by Chiral-Prep-HPLC with the following conditions: Column: CHIRALPAK IE, 2*25 cm, 5 μm; Mobile Phase A: MTBE (0.5% 2M NH₃-MeOH)-HPLC, Mobile Phase B: MeOH-HPLC; Flow rate: 20 mL/min; Gradient: 15% B to 15% B in 17 min; Wave Length: 220/254 nm; RTL 11.557; RT2: 14.464. This resulted in the title compound (ACN-1) (19.6 mg, 37 μmol, 31%) as a white solid. m/z 537.2 (M+H)⁺ (ES+). ¹H NMR (400 MHz, Methanol-d4) δ 8.18 (s, 1H), 8.09 (d, J=7.8 Hz, 1H), 7.91-7.77 (m, 2H), 7.66 (s, 1H), 7.51 (t, J=7.8 Hz, 1H), 7.07-7.02 (m, 1H), 5.21-5.10 (m, 4H), 3.71 (s, 2H), 3.60-3.45 (m, 1H), 2.93 (s, 4H), 2.72-2.57 (m, 2H), 2.42 (d, J=9.3 Hz, 1H), 1.88 (t, J=7.0 Hz, 2H), 1.51 (d, J=6.6 Hz, 3H), 0.64-0.51 (m, 4H).

### Example 146: Synthesis of 2-(3-(3-((4-Methyl-4H-1,2,4-triazol-3-yl)methyl)oxetan-3-yl) phenyl)-7-(trifluoromethyl)benzo[d]oxazole (ACO-1)

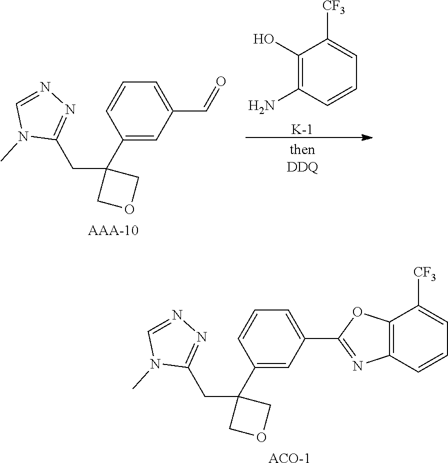

A mixture of intermediate (AAA-10) (50 mg, 1 Eq, 194 μmol) in DCM (6 mL) was treated with intermediate (K-1) (34 mg, 1 Eq, 194 μmol) for 1 hour at 60° C. To the above solution were added DDQ (88 mg, 2 Eq, 389 μmol) at rt. The resulting mixture was stirred for additional 1 hour at rt. The reaction was then quenched by the addition of 3 mL of ice water at 0° C. The resulting mixture was diluted with water and extracted with EtOAc (3×20 mL). The combined organic layers were washed with brine (2×20 mL), dried (Na₂SO₄). After filtration, the filtrate was concentrated in vacuo. The residue was purified by Prep-TLC with DCM/MeOH (8/1). The crude product was further purified by Prep-HPLC with the following conditions (Column: XBridge Prep Phenyl OBD Column, 19*250 mm, 5 μm; Mobile Phase A: Water (0.05% FA), Mobile Phase B: MeCN; Flow rate: 25 mL/min; Gradient: 20% B to 80% B in 7 min; Wave Length: 254/220 nm) to afford the title compound (ACO-1) (19.5 mg, 47 μmol, 24%) as a white solid. m/z 415.3 (M+H)⁺ (ES+). ¹H NMR (400 MHz, DMSO-d6) δ 8.20 (s, 1H), 8.18-8.12 (m, 1H), 8.10-8.03 (m, 1H), 7.87 (t, J=1.8 Hz, 1H), 7.91-7.81 (m, 1H), 7.67-7.53 (m, 2H), 7.36-7.28 (m, 1H), 5.01 (d, J=6.2 Hz, 2H), 4.93 (d, J=6.2 Hz, 2H), 3.60 (s, 2H), 3.02 (s, 3H).

### Example 147: Synthesis of 2-(3-(3-((4-Methyl-4H-1,2,4-triazol-3-yl)methyl)oxetan-3-yl) phenyl)-7-(trifluoromethyl)-1H-indole (ACP-4)

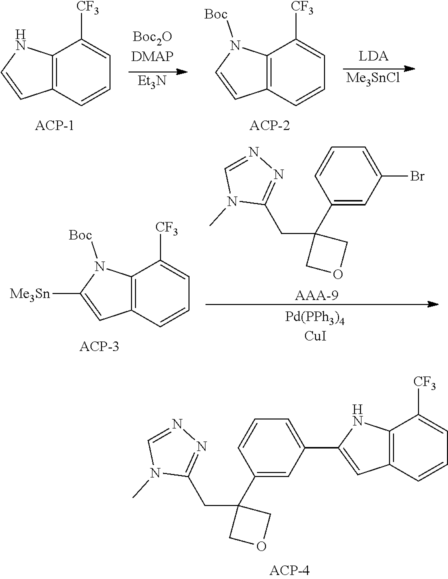

Step 1: tert-Butyl 7-(trifluoromethyl)indole-1-carboxylate (ACP-2). Into a 50-mL round-bottom flask, was placed 7-(trifluoromethyl)-1H-indole (ACP-1) (200 mg, 1 Eq, 1.08 mmol) in THF (10 mL), then Boc₂O (472 mg, 2 Eq, 2.16 mmol), Et₃N (328 mg, 3 Eq, 3.24 mmol) and DMAP (26 mg, 0.2 Eq, 216 μmol) were added at rt. The resulting solution was stirred for 2 h at rt. The resulting mixture was diluted with water and extracted with EtOAc (3×50 mL). The combined organic layers were washed with brine (2×50 mL), dried (Na₂SO₄). After filtration, the filtrate was concentrated in vacuo. The residue was purified by Prep-TLC with EtOAc/petroleum ether (1/10). This resulted in the sub-title compound (ACP-2) (300 mg, 1.05 mmol, 97%) as yellow oil. m/z 286.1 (M+H)⁺ (ES+).

Step 2: tert-Butyl 7-(trifluoromethyl)-2-(trimethylstannyl)indole-1-carboxylate (ACP-3). To a stirred solution of the product from step 1 above (ACP-2) (300 mg, 1 Eq, 1.05 mmol) and trimethyltin chloride (237 mg, 1.13 Eq, 1.19 mmol) in THF (20 mL) was added a solution of LDA (630 μl, 1.2 Eq, 1.26 mmol) dropwise at 0° C. under nitrogen atmosphere. The resulting mixture was stirred for 16 h at rt under nitrogen atmosphere. The resulting mixture was diluted with water and extracted with EtOAc (3×50 mL). The combined organic layers were washed with brine (2×50 mL), dried (Na₂SO₄). After filtration, the filtrate was concentrated in vacuo. The residue was purified by Prep-TLC with EtOAc/petroleum ether (1/10) to afford the sub-title compound (ACP-3) (220 mg, 490 μmol, 47%) as a yellow solid. m/z 450.1 (M+H)⁺ (ES+).

Step 3: 2-(3-(3-((4-Methyl-4H-1,2,4-triazol-3-yl)methyl)oxetan-3-yl)phenyl)-7-(trifluoromethyl)-1H-indole (ACP-4). Into a 30-mL sealed tube purged and maintained with an inert atmosphere of nitrogen, was placed the product from step 2 above (ACP-3) (210 mg, 0.469 mmol, 1 Eq), intermediate (AAA-9) (433 mg, 3 Eq, 1.41 mmol) in DMF (15 mL), then CuI (18 mg, 0.2 Eq, 94 μmol), Pd(PPh₃)₄ (54 mg, 0.1 Eq, 47 μmol) were added at rt under nitrogen atmosphere. The resulting solution was stirred for 16 h at 100° C. under nitrogen atmosphere. The mixture was cooled to rt, diluted with water and extracted with EtOAc (3×50 mL). The combined organic layers were washed with brine (2×50 mL), dried (Na₂SO₄). After filtration, the filtrate was concentrated in vacuo. The crude product was purified by Prep-HPLC with the following conditions (2 #SHIMADZU (HPLC-01)): Column, X Bridge Prep OBD C18 Column, 30*150 mm, 5 μm; mobile phase, Water (0.1% NH₄HCO₃+0.1% NH₃·H₂O) and MeCN (35% Phase B up to 55% in 10 min); Detector, UV 254/220 nm. This resulted in the title compound (ACP-4) (65 mg, 158 μmol, 33%) as a white solid. m/z 411.1 (M−H)⁻ (ES+). ¹H NMR (400 MHz, Methanol-d4) δ 8.18 (s, 1H), 7.82-7.80 (m, 2H), 7.44-7.38 (m, 3H), 7.19-7.15 (m, 1H), 6.87 (s, 1H), 6.84-6.82 (m, 1H), 5.15-5.12 (m, 4H), 3.68 (s, 2H), 2.88 (s, 3H).

### Example 148: Synthesis of 6-(Ethylamino)-4-(1-methyl-4-(4-methyl-4H-1,2,4-triazol-3-yl)-1H-pyrazol-5-yl)-5′-(trifluoromethyl)-[2,3′-bipyridin]-2′(1′H)-one (ACQ-3)

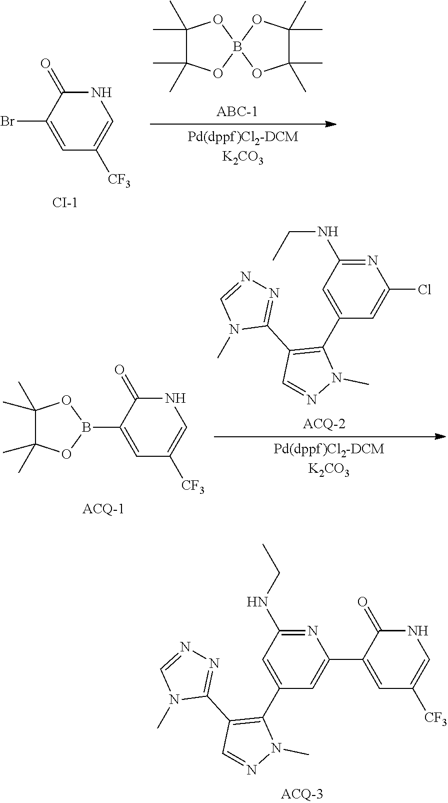

Step 1: 3-(4,4,5,5-Tetramethyl-1,3,2-dioxaborolan-2-yl)-5-(trifluoromethyl)pyridin-2(1H)-one (ACQ-1). To a solution of intermediate (CI-1) (580 mg, 1 Eq, 2.40 mmol), bis(pinacolato)diboron (ABC-1) (1.30 g, 2 Eq, 4.80 mmol) and K₂CO₃ (994 mg, 7.20 mmol, 3 Eq) in THF (15 mL) was added Pd(dppf)Cl₂-DCM (351 mg, 0.2 eq, 500 μmol) at rt under nitrogen atmosphere. After stirring for overnight at 80° C. under a nitrogen atmosphere, the resulting mixture was cooled to rt and concentrated in vacuo. The crude product was purified by reverse flash column chromatography with the following conditions: Column, C18; mobile phase, Water (0.1% FA) and MeCN (10% MeCN up to 60% in 10 min); Detector, UV 254/220 nm. This resulted in the sub-title compound (ACQ-1) (453 mg, 2.19 mmol, 91%) as a brown solid. m/z 290.1 (M+H)⁺ (ES+).

Step 2: 6-(Ethylamino)-4-(1-methyl-4-(4-methyl-4H-1,2,4-triazol-3-yl)-1H-pyrazol-5-yl)-5′-(trifluoromethyl)-[2,3′-bipyridin]-2′(1′H)-one (ACQ-3). To a solution of intermediate (ACQ-2) (50 mg, 1 Eq, 170 μmol), the product from step 1 (ACQ-1) (61 mg, 1.1 Eq, 190 μmol) and K₂CO₃ (72 mg, 520 mmol, 3 Eq) in dioxane (5 mL) and water (1 mL) was added Pd(dppf)Cl₂-DCM (25 mg, 0.2 Eq, 35 μmol) at rt under nitrogen atmosphere. After stirring for overnight at 80° C. under a nitrogen atmosphere, the resulting mixture was cooled to rt and concentrated in vacuo. The crude product was purified by Prep-HPLC with the following conditions (Column: XBridge Prep OBD C18 Column, 30×150 mm, 5 μm; Mobile Phase A: Water (0.1% NH₄HCO₃+0.1% NH₃·H₂O), Mobile Phase B: MeCN; Flow rate: 60 mL/min; Gradient: 19% B to 42% B in 10 min; Wave Length: 254/210 nm; RT: 8.75) to afford the title compound (ACQ-3) (39.5 mg, 89 μmol, 51%) as a yellow green solid. m/z 445.3 (M+H)⁺ (ES+). ¹H NMR (400 MHz, Methanol-d4) δ 8.69 (d, J=2.8 Hz, 1H), 8.47 (s, 1H), 7.94 (s, 1H), 7.86 (s, 1H), 7.78 (d, J=1.3 Hz, 1H), 6.39 (d, J=1.3 Hz, 1H), 4.02 (s, 3H), 3.46 (s, 3H), 3.42-3.35 (m, 2H), 1.24 (t, J=7.2 Hz, 3H).

### Example 149: Synthesis of 6-Chloro-4-(1-methyl-4-(4-methyl-4H-1,2,4-triazol-3-yl)-1H-pyrazol-5-yl)-5′-(trifluoromethyl)-[2,3′-bipyridin]-2′(1′H)-one (ACR-3)

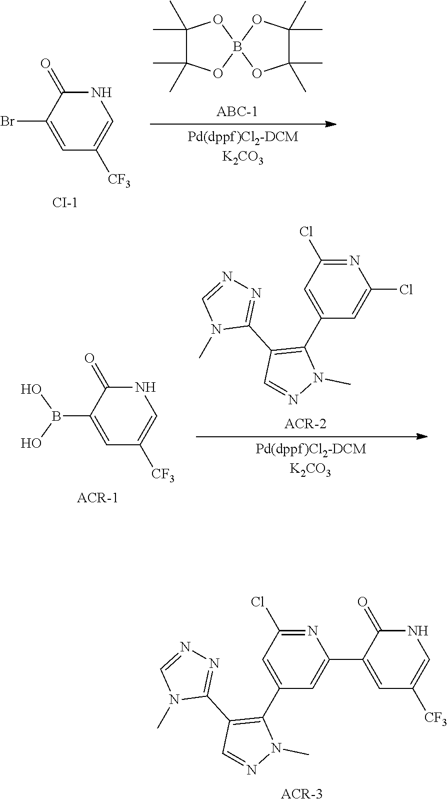

Step 1: (2-Oxo-5-(trifluoromethyl)-1,2-dihydropyridin-3-yl)boronic acid (ACR-1). To a solution of intermediate (CI-1) (580 mg, 1 Eq, 2.40 mmol), bis(pinacolato)diboron (ABC-1) (1.30 g, 2 Eq, 4.80 mmol) and K₂CO₃ (994 mg, 7.20 mmol, 3 Eq) in THF (15 mL) was added Pd(dppf)Cl₂-DCM (351 mg, 0.2 eq, 500 μmol) at rt under nitrogen atmosphere. After stirring overnight at 80° C. under a nitrogen atmosphere, the resulting mixture was cooled to rt and concentrated in vacuo. The crude product was purified by reverse flash column chromatography with the following conditions: Column, C18; mobile phase, Water (0.1% FA) and MeCN (10% MeCN up to 60% in 10 min); Detector, UV 254/220 nm. This resulted in the sub-title compound (ACR-1) (453 mg, 2.19 mmol, 91%) as a brown solid. m/z 208.0 (M+H)⁺ (ES+).

Step 2: 6-Chloro-4-(1-methyl-4-(4-methyl-4H-1,2,4-triazol-3-yl)-1H-pyrazol-5-yl)-5′-(trifluoromethyl)-[2,3′-bipyridin]-2′(1′H)-one (ACR-3). To a solution of the product from step 1 above (ACR-1) (20 mg, 1 Eq, 100 μmol), 2,6-dichloro-4-[2-methyl-4-(4-methyl-1,2,4-triazol-3-yl) pyrazol-3-yl]pyridine (ACR-2) (24 mg, 0.8 Eq, 800 mmol) and K₂CO₃ (40 mg, 3 Eq, 300 μmol) in dioxane (0.8 mL) and water (0.2 mL) was added Pd(dppf)Cl₂-DCM (14 mg, 0.2 Eq, 100 mmol) at rt under nitrogen atmosphere. After stirring for 2 h at 80° C. under a nitrogen atmosphere, the resulting mixture was cooled to rt and concentrated in vacuo. The crude product was purified by Prep-HPLC with the following conditions (Column: XBridge Prep C18 OBD Column, 30*100 mm, 5 μm; Mobile Phase A: Water (0.1% NH₄HCO₃), Mobile Phase B: MeCN; Flow rate: 60 mL/min; Gradient: 12% B to 35% B in 10 min; Wave Length: 254/220 nm; RT: 10.38) to afford the title compound (ACR-3) (9.5 mg, 22 μmol, 23%) as an off-white solid. m/z 436.0 (M+H)⁺ (ES+). ¹H NMR (400 MHz, Methanol-d4) δ 8.66 (d, J=2.8 Hz, 1H), 8.57 (d, J=1.3 Hz, 1H), 8.50 (s, 1H), 8.02 (s, 1H), 7.95 (s, 1H), 7.50 (d, J=1.3 Hz, 1H), 4.04 (s, 3H), 3.64 (s, 3H).

### Example 150: Synthesis of N-Ethyl-4-(1-methyl-4-(4-methyl-4H-1,2,4-triazol-3-yl)-1H-pyrazol-5-yl)-6-(7-(trifluoromethyl)-1H-benzo[d]imidazol-2-yl)pyridin-2-amine (ACS-3)

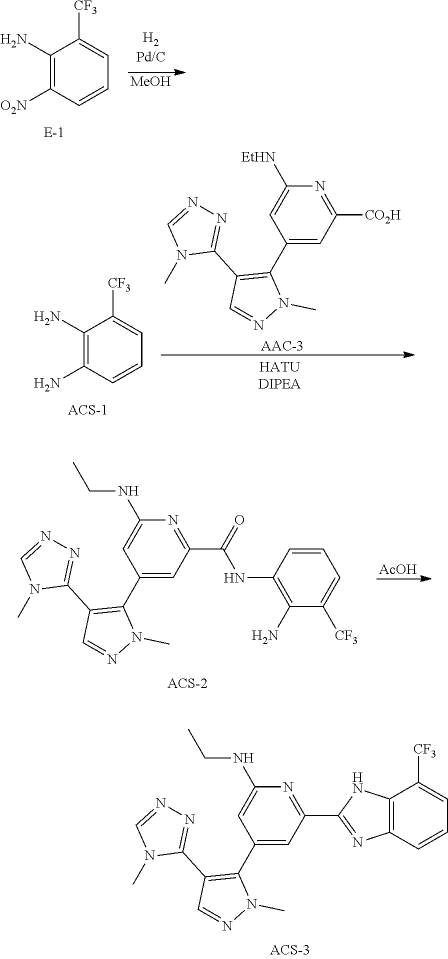

Step 1: 3-(Trifluoromethyl) benzene-1,2-diamine (ACS-1). Into a 25 mL round-bottom flask were added intermediate (E-1) (150 mg, 1 Eq, 728 μmol) in MeOH (3 mL) at rt. To the above mixture was added Pd/C (10% Wt, 100 mg) at rt under nitrogen atmosphere. The resulting mixture was stirred for additional 3 h at rt under hydrogen atmosphere. The resulting mixture was filtered; the filter cake was washed with MeOH (3×3 mL). The filtrate was concentrated in vacuo to afford the sub-title compound (ACS-1) (101 mg, 574 μmol, 79%) as colorless oil. m/z 177.1 (M+H)⁺ (ES+).

Step 2: N-(2-Amino-3-(trifluoromethyl)phenyl)-6-(ethylamino)-4-(1-methyl-4-(4-methyl-4H-1,2,4-triazol-3-yl)-1H-pyrazol-5-yl)picolinamide (ACS-2). Into an 8 mL sealed tube were added intermediate (AAC-3) (70 mg, 1 Eq, 214 μmol), the product from step 2 above (ACS-1) (45 mg, 1.2 Eq, 257 μmol) and DIPEA (83 mg, 3 Eq, 642 μmol) in DMF (3 mL) at rt. To the above mixture was added HATU (81 mg, 1 Eq, 214 μmol) over 3 min at 0° C. The resulting mixture was stirred for additional 2 h at rt. The crude product was purified by reverse flash column chromatography with the following conditions: Column, C18; mobile phase, Water (0.1% NH₄HCO₃) and MeCN (10% MeCN up to 70% in 20 min); Detector, UV 254/220 nm. The product-containing fractions were combined and concentrated in vacuo. This resulted in the sub-title compound (ACS-2) (51 mg, 105 μmol, 49%) as yellow oil. m/z 486.2 (M+H)⁺ (ES+).

Step 3: N-Ethyl-4-(1-methyl-4-(4-methyl-4H-1,2,4-triazol-3-yl)-1H-pyrazol-5-yl)-6-(7-(trifluoromethyl)-1H-benzo[d]imidazol-2-yl)pyridin-2-amine (ACS-3). Into an 8 mL sealed tube were added the product from step 2 above (ACS-2) (40 mg, 1 Eq, 82 μmol) in AcOH (2 mL) at rt. The resulting mixture was stirred overnight at 80° C. The mixture was cooled to rt and concentrated in vacuo. The crude product was purified by Prep-HPLC with the following conditions: Column: SunFire Prep C18 OBD Column, 19×150 mm, 5 μm; Mobile Phase A: Water (0.1% NH₄HCO₃+0.1% NH₃·H₂O), Mobile Phase B: MeCN; Flow rate: 60 mL/min; Gradient: 30 B to 60 B in 7 min; Detector, UV 254/210 nm; RT: 6.08 to afford the title compound (ACS-3) (14.4 mg, 31 μmol, 37%) as a white solid. m/z 468.0 (M+H)⁺ (ES+). ¹H NMR (400 MHz, Methanol-d4) δ 8.48 (s, 1H), 7.90 (d, J=7.8 Hz, 2H), 7.70-7.54 (m, 2H), 7.42 (t, J=7.9 Hz, 1H), 6.48 (s, 1H), 4.05 (s, 3H), 3.52 (d, J=7.5 Hz, 5H), 1.27 (t, J=7.2 Hz, 3H).

### Example 151: Synthesis of 6-(6-Bromo-7-(trifluoromethyl)-1H-benzo[d]imidazol-2-yl)-N-ethyl-4-(1-methyl-4-(4-methyl-4H-1,2,4-triazol-3-yl)-1H-pyrazol-5-yl) pyridin-2-amine (ACT-3)

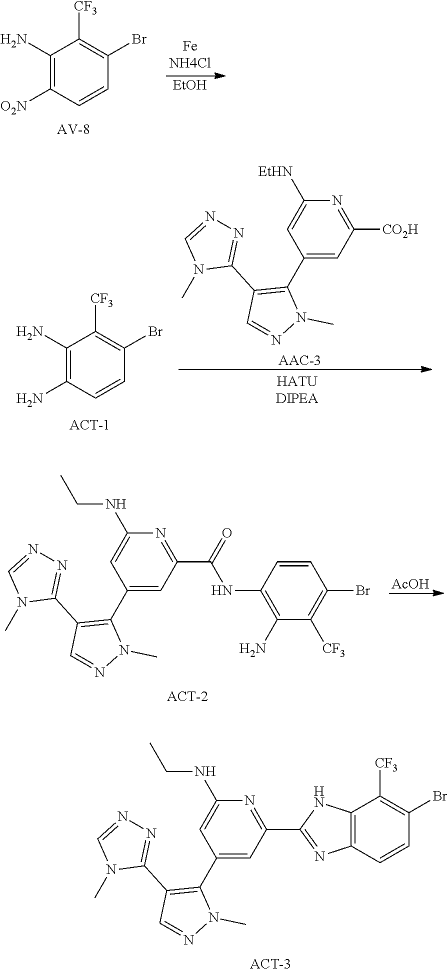

Step 1: 4-Bromo-3-(trifluoromethyl)benzene-1,2-diamine (ACT-1). To a stirred solution o 3-bromo-6-nitro-2-(trifluoromethyl) aniline (AV-8) (140 mg, 1 Eq, 491 μmol) in EtOH (10 mL) and water (2 mL) was added Fe powder (137 mg, 5 Eq, 2.46 mmol) and NH₄Cl (263 mg, 10 Eq, 4.91 mmol) at rt under nitrogen atmosphere. The resulting mixture was stirred for 2 h at 80° C. under nitrogen atmosphere. The mixture was cooled to rt and concentrated in vacuo. The residue was purified by Prep-TLC with EtOAc/petroleum ether (1/2) to afford the sub-title compound (ACT-1) (100 mg, 394 μmol, 80%) as a brown solid. m/z 255.0/257.0 (M+H)⁺ (ES+).

Step 2: N-(2-Amino-4-bromo-3-(trifluoromethyl)phenyl)-6-(ethylamino)-4-(1-methyl-4-(4-methyl-4H-1,2,4-triazol-3-yl)-1H-pyrazol-5-yl)picolinamide (ACT-2). Into an 8 mL sealed tube were added intermediate (AAC-3) (60 mg, 1 Eq, 183 μmol), the product from step 1 above (ACT-1) (56 mg, 1.2 Eq, 220 μmol) and DIPEA (71 mg, 3 Eq, 549 μmol) in DMF (2 mL) at rt. To the above mixture was added HATU (70 mg, 1 Eq, 183 μmol) over 3 min at 0° C. The resulting mixture was stirred for additional 3 h at rt. The crude product was purified by reverse flash column chromatography with the following conditions: Column, C18; mobile phase, Water (0.1% NH₄HCO₃) and MeCN (10% MeCN up to 70% in 15 min); Detector, UV 254/220 nm. The product-containing fractions were combined and concentrated in vacuo to afford the sub-title compound (ACT-2) (49 mg, 87 μmol, 47%) as a colorless oil. m/z 564.1/566.1 (M+H)⁺ (ES+).

Step 3: 6-(6-Bromo-7-(trifluoromethyl)-1H-benzo[d]imidazol-2-yl)-N-ethyl-4-(1-methyl-4-(4-methyl-4H-1,2,4-triazol-3-yl)-1H-pyrazol-5-yl) pyridin-2-amine (ACT-3). Into an 8 mL sealed tube were added the product from step 2 above (ACT-2) (40 mg, 1 Eq, 71 μmol) in AcOH (3 mL) at rt. The resulting mixture was stirred for additional 2 h at 80° C. The mixture was cooled to rt and concentrated in vacuo. The crude product was purified by Prep-HPLC with the following conditions: Column: SunFire Prep C18 OBD Column, 19×150 mm, 5 μm; Mobile Phase A: Water (0.1% NH₄HCO₃+0.1% NH₃·H₂O), Mobile Phase B: MeCN; Flow rate: 60 mL/min; Gradient: 30 B to 60 B in 7 min; Detector, UV 254/210 nm; RT: 6.08 to afford the title compound (ACT-3) (16.2 mg, 30 μmol, 42%) as a white solid. m/z 546.0/548.0 (M+H)⁺ (ES+). ¹H NMR (400 MHz, Methanol-d4) δ 8.49 (s, 1H), 8.01-7.36 (m, 4H), 6.54 (d, J=25.4 Hz, 1H), 4.04 (s, 3H), 3.53 (d, J=9.7 Hz, 5H), 1.28 (t, J=7.2 Hz, 3H).

### Example 152: Synthesis of N-Ethyl-4-[2-methyl-4-(4-methyl-1,2,4-triazol-3-yl)-pyrazol-3-yl]-6-[7-(trifluoromethyl)-1,3-benzothiazol-2-yl]-pyridin-2-amine (ACU-2)

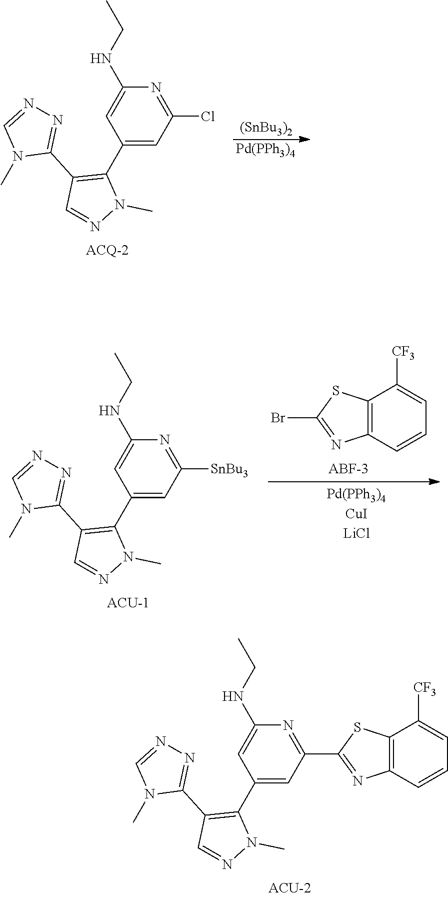

Step 1: N-Ethyl-4-[2-methyl-4-(4-methyl-1,2,4-triazol-3-yl)-pyrazol-3-yl]-6-(tributylstannyl)-pyridin-2-amine (ACU-1). To a stirred solution of intermediate (ACQ-2) (30 mg, 1 Eq, 90 μmol) and hexabutyldistannane (164 mg, 3 Eq, 280 μmol) in toluene (3 mL) was added Pd(PPh₃)₄ (11 mg, 0.1 Eq, 10 mmol) at rt under nitrogen atmosphere. The resulting mixture was stirred for 16 hours at 120° C. under nitrogen atmosphere. The mixture was cooled to rt and concentrated in vacuo. The residue was purified by Prep-TLC with DCM/MeOH (10/1) to afford the sub-title compound (ACU-1) (17 mg, 30 μmol, 31%) as a white solid. m/z 574.3 (M+H)⁺ (ES+).

Step 2: N-Ethyl-4-[2-methyl-4-(4-methyl-1,2,4-triazol-3-yl)-pyrazol-3-yl]-6-[7-(trifluorome thyl)-1,3-benzothiazol-2-yl]-pyridin-2-amine (ACU-2). To a stirred mixture of the product from step 1 above (ACU-1) (34 mg, 1 Eq, 50 μmol) and intermediate (ABF-3) (17 mg, 1 Eq, 50 mmol) in 1,4-dioxane (5 mL) were added LiCl (4 mg, 1.5 Eq, 80 μmol) and CuI (5 mg, 0.4 Eq, 20 μmol) and Pd(PPh₃)₄ (14 mg, 0.2 Eq, 10 μmol) at rt under nitrogen atmosphere. The resulting mixture was stirred for 16 hours at 100° C. under nitrogen atmosphere. The resulting mixture was cooled to rt. The resulting mixture was concentrated in vacuo. The residue was purified by Prep-TLC with DCM/MeOH (10/1). The crude product was purified by Prep-HPLC with the following conditions: Column: SunFire Prep C18 OBD Column, 30×150 mm, 5 μm; Mobile Phase A: Water (0.1% NH₄HCO₃), Mobile Phase B: MeCN; Flow rate: 60 mL/min; Gradient: 37 B to 60 B in 8 min; Detector, UV 254/210 nm; RT: 7.36. The product-containing fractions were combined and evaporated partially in vacuo and lyophilized overnight to afford the title compound (ACU-2) (5.5 mg, 11 μmol, 18%) as an off-white solid. m/z 485.0 (M+H)⁺ (ES+). ¹H NMR (400 MHz, Methanol-d4) δ 8.49 (s, 1H), 8.25 (d, J=8.1 Hz, 1H), 7.92 (s, 1H), 7.82 (d, J=7.6 Hz, 1H), 7.72 (t, J=7.9 Hz, 1H), 7.50 (d, J=1.3 Hz, 1H), 6.58 (d, J=1.3 Hz, 1H), 4.04 (s, 3H), 3.53-3.44 (m, 5H), 1.30 (t, J=7.2 Hz, 3H).

### Example 153: Synthesis of N-Ethyl-4-(1-methyl-4-(4-methyl-4H-1,2,4-triazol-3-yl)-1H-pyrazol-5-yl)-6-(6-methyl-7-(trifluoromethyl)-1H-benzo[d]imidazol-2-yl) pyridin-2-amine (ACV-5)

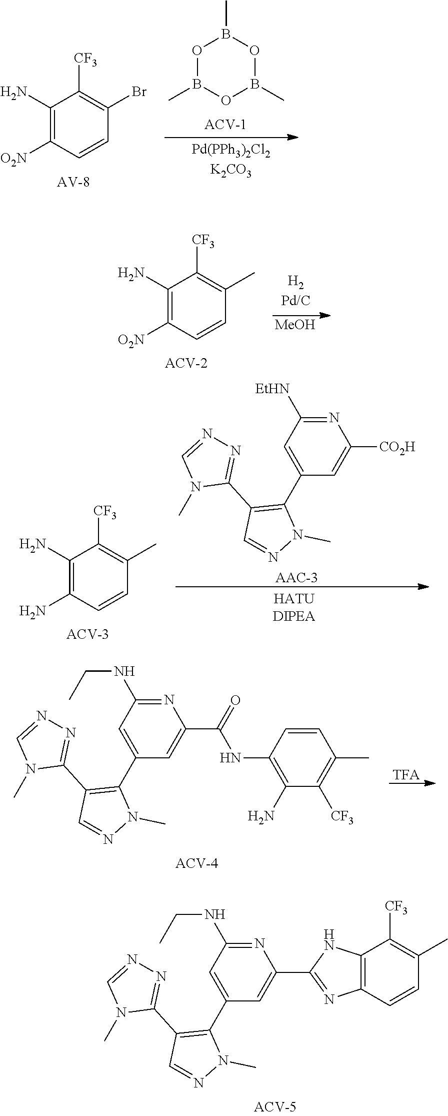

Step 1: 3-methyl-6-nitro-2-(trifluoromethyl)aniline (ACV-2). To a stirred mixture of intermediate (AV-8) (143 mg, 1 Eq, 502 μmol), trimethyl-1,3,5,2,4,6-trioxatriborinane (ACV-1) (63 mg, 1 Eq, 502 μmol) and K₂CO₃ (208 mg, 1.51 mmol, 3 Eq) in dioxane (5 mL) was added Pd(PPh₃)₂Cl₂ (35 mg, 0.1 Eq, 50 μmol) at rt under nitrogen atmosphere. The resulting mixture was stirred for overnight at 110° C. under nitrogen atmosphere. The mixture was cooled to rt and concentrated in vacuo. The crude product was purified by reverse flash column chromatography with the following conditions: Column, C18; mobile phase, Water (0.1% NH₄HCO₃) and MeCN (55% MeCN up to 70% in 10 min); Detector, UV 254/220 nm. The product-containing fractions were combined and concentrated in vacuo. This resulted in the sub-title compound (ACV-2) (95 mg, 432 μmol, 86%) as a yellow solid. m/z 221.0 (M+H)⁺ (ES+).

Step 2: 4-Methyl-3-(trifluoromethyl)benzene-1,2-diamine (ACV-3). Into a 25 mL round-bottom flask were added the product from step 1 above (ACV-2) (88 mg, 1 Eq, 400 μmol) in MeOH (3 mL) at rt. To the above mixture was added Pd/C (10% Wt, 80 mg) at rt under nitrogen atmosphere. The resulting mixture was stirred for additional 3 h at rt under hydrogen atmosphere. The resulting mixture was filtered; the filter cake was washed with MeOH (3×3 mL). The filtrate was concentrated in vacuo to afford the sub-title compound (ACV-3) (55 mg, 289 μmol, 72%) as a colorless oil. m/z 191.1 (M+H)⁺ (ES+).

Step 3: N-(2-Amino-4-methyl-3-(trifluoromethyl)phenyl)-6-(ethylamino)-4-(1-methyl-4-(4-methyl-4H-1,2,4-triazol-3-yl)-1H-pyrazol-5-yl)picolinamide (ACV-4). To a stirred mixture of intermediate (AAC-3) (25 mg, 1 Eq, 76 μmol) and the product from step 2 above (ACV-3) (17 mg, 1.2 Eq, 92 μmol) in DMF (1 mL) were added HATU (29 mg, 1 Eq, 76 μmol) and DIPEA (30 mg, 3 Eq, 229 μmol) at 0° C. under nitrogen atmosphere. The resulting mixture was stirred overnight at rt under nitrogen atmosphere. The crude product was purified by reverse flash column chromatography with the following conditions: Column, C18; mobile phase, Water (0.1% NH₄HCO₃) and MeCN (55% MeCN up to 70% in 10 min); Detector, UV 254/220 nm. The product-containing fractions were combined and concentrated in vacuo. This resulted in the sub-title compound (ACV-4) (20 mg, 40 μmol, 52%) m/z 500.2 (M+H)⁺ (ES+).

Step 4: N-Ethyl-4-(1-methyl-4-(4-methyl-4H-1,2,4-triazol-3-yl)-1H-pyrazol-5-yl)-6-(6-methyl-7-(trifluoromethyl)-1H-benzo[d]imidazol-2-yl) pyridin-2-amine (ACV-5). A solution of the product from step 3 above (ACV-4) (24 mg, 1 Eq, 48 μmol) in AcOH (5 mL) was stirred for 2 h at 80° C. under nitrogen atmosphere. The mixture was cooled to rt and concentrated in vacuo. The crude product was purified by Prep-HPLC with the following conditions (Column: Sunfire prep C18 column, 30*150 mm, 5 μm; Mobile Phase A: Water (0.1% FA), Mobile Phase B: MeCN; Flow rate: 60 mL/min; Gradient: 33% B to 57% B in 7 min; Wave Length: 254/220 nm; RT: 6.18) to afford the title compound (ACV-5) (5.7 mg, 12 μmol, 24%) as a white solid. m/z 482.3 (M+H)⁺ (ES+). ¹H NMR (400 MHz, Methanol-d4) δ 8.48 (s, 1H), 7.91 (s, 1H), 7.78 (s, 1H), 7.45 (s, 1H), 7.29 (s, 1H), 6.52 (s, 1H), 4.04 (s, 3H), 3.50 (d, J=21.3 Hz, 5H), 2.67-2.55 (m, 3H), 1.28 (t, J=7.2 Hz, 3H).

### Example 154: Synthesis of N-Ethyl-4-(1-methyl-4-(4-methyl-4H-1,2,4-triazol-3-yl)-1H-pyrazol-5-yl)-6-(7-(trifluoromethyl)benzo[d]oxazol-2-yl)pyridin-2-amine (ACW-2)

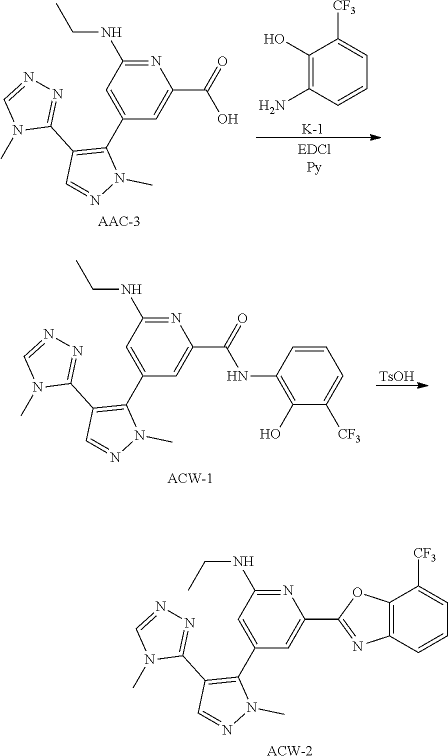

Step 1: 6-(Ethylamino)-N-[2-hydroxy-3-(trifluoromethyl)-phenyl]-4-[2-methyl-4-(4-methyl-1,2,4-triazol-3-yl)-pyrazol-3-yl]-pyridine-2-carboxamide (ACW-1). To a stirred solution of intermediate (AAC-3) (25 mg, 1 Eq, 70 μmol) and intermediate (K-1) (27 mg, 2 Eq, 150 μmol) in pyridine (5 mL) was added EDCI (29 mg, 2 Eq, 150 μmol) at rt. The resulting mixture was stirred for 2 hours at 80° C. The mixture was cooled to rt. The resulting mixture was concentrated in vacuo. The residue was purified by Prep-TLC with DCM/MeOH (15/1) to afford the sub-title compound (ACW-1) (34 mg, 70 μmol, 91%) as a white solid. m/z 487.2 (M+H)⁺ (ES+).

Step 2: N-Ethyl-4-[2-methyl-4-(4-methyl-1,2,4-triazol-3-yl)-pyrazol-3-yl]-6-[7-(trifluorome thyl)-1,3-benzoxazol-2-yl]-pyridin-2-amine (ACW-2). The product from step 1 above (ACW-1) (30 mg, 1 Eq, 60 μmol) and pTsOH (11 mg, 1 Eq, 60 mmol) were added in toluene (2 mL) at rt under nitrogen atmosphere. The resulting mixture was stirred for 72 hours at 100° C. under nitrogen atmosphere. The resulting mixture was cooled to rt. The resulting mixture was concentrated in vacuo. The crude product was purified by reverse flash column chromatography with the following conditions: Column, C18; mobile phase, Water (0.1% NH₄HCO₃) and MeCN (30% MeCN up to 45% in 10 min); Detector, UV 254/220 nm. The product-containing fractions were combined and concentrated in vacuo. The crude product was purified by Prep-HPLC with the following conditions (Column: XBridge Prep OBD C18 Column, 30*150 mm, 5 μm; Mobile Phase A: Water (0.1% NH₄HCO₃), Mobile Phase B: MeCN; Flow rate: 60 mL/min; Gradient: 26% B to 50% B in 10 min, Wave Length: 254/210 nm; RT: 9.17). The product-containing fractions were combined and evaporated partially in vacuo and lyophilized overnight to afford the title compound (ACW-2) (5.3 mg, 11 μmol, 18%) as a white solid. m/z 469.1 (M+H)⁺ (ES+). ¹H NMR (400 MHz, Methanol-d4) δ 8.49 (s, 1H), 8.06 (d, J=8.0 Hz, 1H), 7.92 (s, 1H), 7.76 (d, J=7.8 Hz, 1H), 7.61 (t, J=7.9 Hz, 1H), 7.44 (d, J=1.3 Hz, 1H), 6.66 (d, J=1.3 Hz, 1H), 4.04 (s, 3H), 3.55 (s, 3H), 3.51-3.43 (m, 2H), 1.28 (t, J=7.2 Hz, 3H).

### Example 155: 1-((((2-(4′-Fluoro-2′-(4-methyl-4H-1,2,4-triazol-3-yl)-[1,1′-biphenyl]-3-yl)-7-(trifluoromethyl)-1H-benzo[d]imidazol-5-yl)methyl)amino)methyl)cyclopentan-1-ol (CJ-1)

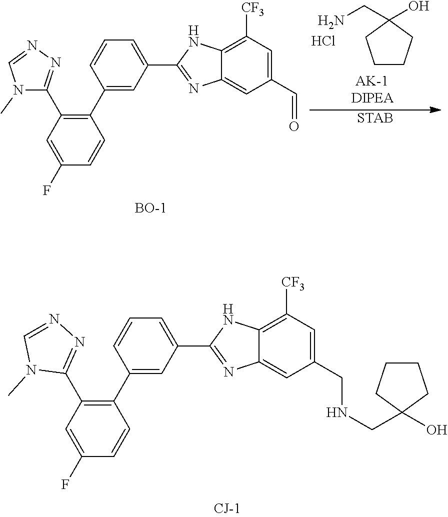

A solution of intermediate (BO-1) (20 mg, 95% Wt, 1 Eq, 41 μmol), 1-(aminomethyl)cyclopentan-1-ol, HCl (AK-1) (12 mg, 2 Eq, 82 μmol) and DIPEA (16 mg, 21 μL, 3.0 Eq, 0.12 mmol) in CHCl₃ (1 mL) was stirred for 1 h at rt. NaBH(OAc)₃ (26 mg, 3 Eq, 0.12 mmol) was added and the reaction mixture was stirred at rt overnight. The reaction mixture was diluted with EtOAc (10 mL) and washed with sat. aq. sol. of NaHCO₃ (2×5 mL) and brine (5 mL). The organic layer was extracted, dried (MgSO₄), filtered and concentrated in vacuo. The crude product was purified by chromatography on silica gel (12 g cartridge, 0-50% MeOH/DCM) to afford the title compound (CJ-1) (14.09 mg, 24 μmol, 58%, 95% Purity) as a pale tan solid. m/z 565.5 (M+H)⁺ (ES+). ¹H NMR (400 MHz, DMSO-d6) δ 8.40 (s, 1H), 8.12 (d, J=7.9 Hz, 1H), 8.08 (s, 1H), 7.83 (s, 1H), 7.78 (dd, J=8.7, 5.7 Hz, 1H), 7.67-7.57 (m, 2H), 7.53 (dd, J=9.2, 2.7 Hz, 1H), 7.46 (t, J=7.8 Hz, 1H), 7.11 (d, J=7.8 Hz, 1H), 4.01 (s, 2H), 3.17 (s, 3H), 2.61 (s, 2H), 1.74-1.62 (m, 2H), 1.62-1.43 (m, 6H). Three exchangeable protons not observed.

### Example 156: 3,3-Difluoro-1-((((2-(4′-fluoro-2′-(4-methyl-4H-1,2,4-triazol-3-yl)-[1,1′-biphenyl]-3-yl)-7-(trifluoromethyl)-1H-benzo[d]imidazol-5-yl)methyl)amino)methyl)cyclobutan-1-ol (CK-2)

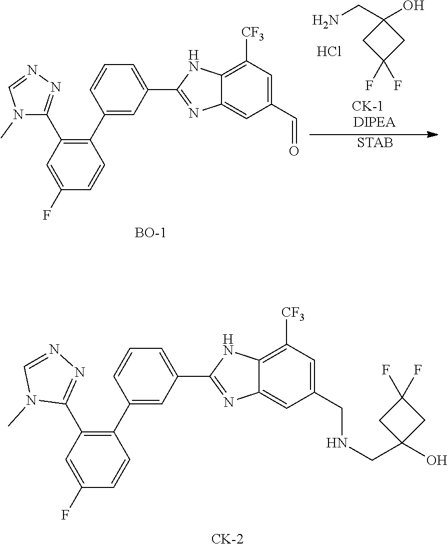

A solution of intermediate (BO-1) (30 mg, 95% Wt, 1 Eq, 61 μmol), 1-(aminomethyl)-3,3-difluorocyclobutan-1-ol, HCl (CK-1) (21 mg, 2 Eq, 0.12 mmol) and DIPEA (24 mg, 32 μL, 3.0 Eq, 0.18 mmol) in CHCl₃ (1 mL) was stirred for 1 h at rt. NaBH(OAc)₃ (39 mg, 3 Eq, 0.18 mmol) was added and the reaction mixture was stirred at rt overnight. The reaction mixture was diluted with EtOAc (10 mL) and washed with sat. aq. sol. of NaHCO₃ (2×5 mL) and brine (5 mL). The organic layer was extracted, dried (MgSO₄), filtered and concentrated in vacuo. The crude product was purified by chromatography on silica gel (12 g cartridge, 0-10% MeOH/DCM) to afford the title compound (CK-2) (22.02 mg, 36 μmol, 58%, 95% Purity) as a pale tan solid. m/z 587.2 (M+H)⁺ (ES+). ¹H NMR (400 MHz, DMSO-d6) δ 8.40 (s, 1H), 8.11 (d, J=7.9 Hz, 1H), 8.07 (s, 1H), 7.81-7.74 (m, 2H), 7.62 (td, J=8.6, 2.8 Hz, 1H), 7.57-7.50 (m, 2H), 7.46 (t, J=7.8 Hz, 1H), 7.10 (d, J=7.8 Hz, 1H), 5.43 (s, 1H), 3.94 (s, 2H), 3.16 (s, 3H), 2.78-2.64 (m, 2H), 2.60 (s, 2H). CH₂ masked by DMSO signal, and two exchangeable protons not observed.

### Example 157: 3′-(5-(Hydroxymethyl)-7-(trifluoromethyl)benzo[d]oxazol-2-yl)-2-(4-methyl-4H-1,2,4-triazol-3-yl)-[1,1′-biphenyl]-4-carbonitrile (CL-2)

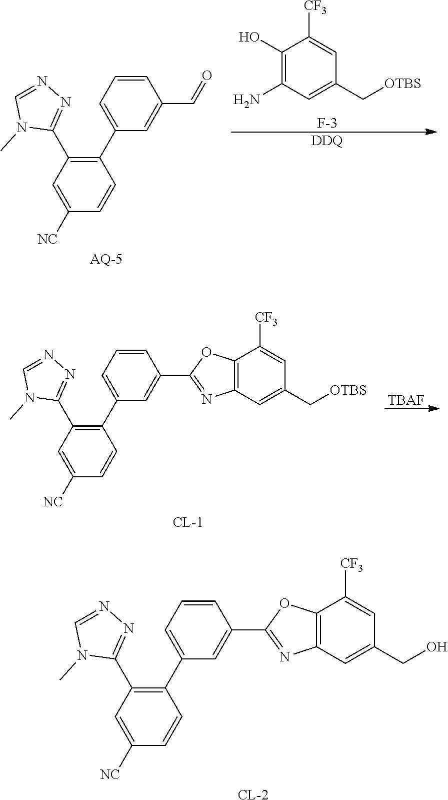

Step 1: 3′-(5-(((tert-Butyldimethylsilyl)oxy)methyl)-7-(trifluoromethyl)benzo[d]oxazol-2-yl)-2-(4-methyl-4H-1,2,4-triazol-3-yl)-[1,1′-biphenyl]-4-carbonitrile (CL-1). To a solution of intermediate (AQ-5) (106 mg, 95% Wt, 1 Eq, 349 μmol) in MeOH (1 mL) was added intermediate (F-3) (145 mg, 85% Wt, 1.1 Eq, 384 μmol). The resulting mixture was stirred at 45° C. for 12 h. After concentration in vacuo, the residue was dissolved in DCM (2 mL) and DDQ (87.2 mg, 1.1 Eq, 384 μmol) was then added. The mixture was stirred 16h at rt. Two additional equivalents of DDQ were added. The resulting mixture was diluted with additional EtOAc (25 mL) and washed sequentially with saturated NaHCO₃ (2×10 mL) and brine (10 mL). The organic layer was dried (Na₂SO₄) and the mixture adsorbed onto silica gel and concentrated in vacuo. The crude product was purified by chromatography on silica gel (24 g cartridge, 0-20% MeOH/DCM) to afford the sub-title compound (CL-1) (164 mg, 0.19 mmol, 56%, 70% Purity). m/z 590.2 (M+H)⁺ (ES+).

Step 2: 3′-(5-(Hydroxymethyl)-7-(trifluoromethyl)benzo[d]oxazol-2-yl)-2-(4-methyl-4H-1,2,4-triazol-3-yl)-[1,1′-biphenyl]-4-carbonitrile (CL-2). To a solution of the product from step 1 above (CL-1) (164 mg, 70% Wt, 1 Eq, 195 μmol) in THF (2 mL) was added TBAF (76.4 mg, 292 μL, 1 molar, 1.5 Eq, 292 μmol). The mixture was stirred at rt for 2 h. The reaction mixture was diluted with EtOAc (25 mL) and transferred into a separating funnel. The layer was washed with sat. aq. sol. of NaHCO₃ (2×5 mL), sat. aq. sol. of NH₄Cl (2×5 mL) then brine. The combined organic layers were collected, dried (MgSO₄), filtered and concentrated in vacuo. The crude product was purified by chromatography on RP Flash C18 (12 g cartridge, 15-75% MeCN/10 mM ammonium bicarbonate) to afford the title compound (CL-2) (51 mg, 0.11 mmol, 55%, 99% Purity) as a pale-yellow solid. m/z 476.2 (M+H)⁺ (ES+). ¹H NMR (400 MHz, Methanol-d4) δ 8.45 (s, 1H), 8.30 (d, J=7.8 Hz, 1H), 8.17 (dd, J=8.1, 1.8 Hz, 1H), 8.15-8.09 (m, 2H), 8.02 (s, 1H), 7.98 (d, J=8.1 Hz, 1H), 7.75 (s, 1H), 7.66 (t, J=7.8 Hz, 1H), 7.52 (d, J=7.7 Hz, 1H), 4.82 (s, 2H), 3.29 (s, 3H). Exchangeable proton not visible.

### Example 158: (1S,2R)-2-(((2-(4′-Fluoro-2′-(4-methyl-4H-1,2,4-triazol-3-yl)-[1,1′-biphenyl]-3-yl)benzo[d]oxazol-5-yl)methyl)amino)cyclopentan-1-ol (CM-1)

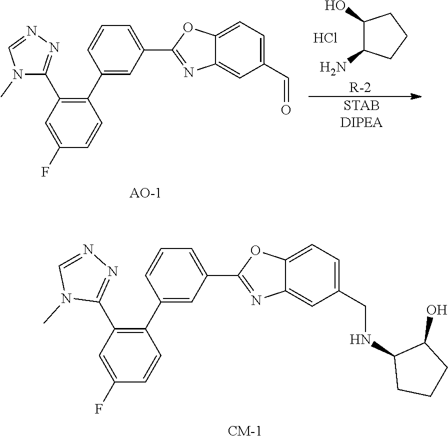

A solution of intermediate (AO-1) (63.0 mg, 91% Wt, 1 Eq, 144 μmol), (1S,2R)-2-aminocyclopentan-1-ol, HCl (R-2) (39.6 mg, 2.0 Eq, 288 μmol) and DIPEA (55.8 mg, 75.2 μL, 3.0 Eq, 432 μmol) in CHCl₃ (1 mL) was stirred 1 h at 40° C., then NaBH(OAc)₃ (152 mg, 5.0 Eq, 720 μmol) was added. The reaction mixture was stirred at 40° C. overnight. The reaction mixture was diluted with EtOAc (25 mL) and transferred into a separating funnel. The layer was washed with sat. aq. sol. of NaHCO₃ (2×5 mL) and brine. The combined organic layers were collected, dried (MgSO₄), filtered and concentrated in vacuo. The crude was purified by preparative HPLC (Waters, Basic (0.1% ammonia), Waters XBridge BEH C18 ODB, 5 μm, 30×100 mm column 20-100% MeCN in water) to afford the title compound (CM-1) (21 mg, 43 μmol, 30%, 99% Purity). m/z 484.2 (M+H)⁺ (ES+). ¹H NMR (400 MHz, Methanol-d4) δ 8.40 (s, 1H), 8.22 (dt, J=7.9, 1.3 Hz, 1H), 8.10 (t, J=1.9 Hz, 1H), 7.83-7.76 (m, 2H), 7.66 (d, J=8.4 Hz, 1H), 7.57 (td, J=8.2, 2.7 Hz, 2H), 7.49 (dd, J=8.7, 2.3 Hz, 2H), 7.42 (dt, J=7.8, 1.4 Hz, 1H), 4.19 (td, J=4.3, 2.2 Hz, 1H), 4.01 (d, J=12.9 Hz, 1H), 3.92 (d, J=12.9 Hz, 1H), 3.21 (s, 3H), 2.99 (td, J=8.3, 7.5, 4.1 Hz, 1H), 2.01-1.68 (m, 4H), 1.66-1.49 (m, 2H). Exchangeable protons not visible

### Example 159: (1R,2S)—N-((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)-2-methoxy-N-methylcyclopentan-1-amine (CN-5)

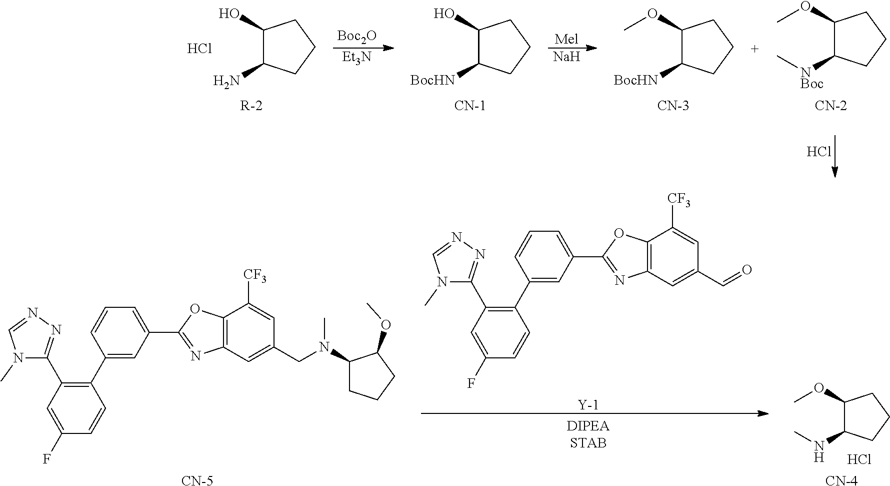

Step 1: tert-Butyl ((1R,2S)-2-hydroxycyclopentyl)carbamate (CN-1). Boc₂0 (1.87 g, 1.2 Eq, 8.55 mmol), (1S,2R)-2-aminocyclopentan-1-ol, HCl (R-2) (980 mg, 1 Eq, 7.12 mmol) and Et₃N (1.08 g, 1.49 mL, 1.5 Eq, 10.7 mmol) in DCM (20 mL) were stirred at rt over the weekend. The reaction mixture was diluted with DCM (100 mL) and washed with aq. 1M HCl (100 mL). The organic layer was passed through a phase separator and the bulk solvent removed in vacuo to afford the sub-title compound (CN-1) (1.32 g, 6.3 mmol, 88%, 96% Purity) as a colourless oil. ¹H NMR (400 MHz, DMSO-d6) δ 6.08 (d, J=7.9 Hz, 1H), 4.56-4.51 (m, 1H), 3.91-3.84 (m, 1H), 3.59-3.49 (m, 1H), 1.78-1.58 (m, 3H), 1.55-1.40 (m, 3H), 1.38 (s, 9H).

Step 2: tert-Butyl ((1R,2S)-2-methoxycyclopentyl)(methyl)carbamate (CN-2) and tert-butyl ((1R,2S)-2-methoxycyclopentyl)carbamate (CN-3). NaH (881 mg, 60% Wt, 3.5 Eq, 22.0 mmol) was added to the product from step 1 above (CN-1) (1.32 g, 96% Wt, 1 Eq, 6.30 mmol) and iodomethane (983 mg, 431 μL, 1.1 Eq, 6.93 mmol) in DMF (20 mL) at 0° C. The reaction mixture was stirred at rt overnight. The reaction mixture was poured into water (100 mL) and extracted with EtOAc (50 mL). The organic extract was washed with 1:1 v/v water-brine (100 mL), dried (MgSO₄), filtered and evaporated to a colourless oil (1.22 g). The colourless oil (700 mg) was purified by chromatography on silica gel (40 g cartridge, 0-20% EtOAc in DCM) to afford tert-butyl ((1R,2S)-2-methoxycyclopentyl)(methyl)carbamate (CN-2) (83 mg, 0.33 mmol, 5.2%, 90% Purity) as a colourless oil. 1H NMR (400 MHz, DMSO-d6) δ 3.70-3.62 (m, 1H), 3.32-3.30 (m, 1H), 3.18 (dd, J=2.8, 1.3 Hz, 3H), 2.77 (dd, J=2.7, 1.3 Hz, 3H), 1.82-1.54 (m, 5H), 1.46-1.36 (m, 10H). tert-butyl ((1R,2S)-2-methoxycyclopentyl)carbamate (CN-3) (112 mg, 0.47 mmol, 7.4%, 90% Purity) as a colourless oil. 1H NMR (400 MHz, DMSO-d6) δ 6.32 (d, J=8.2 Hz, 1H), 3.75-3.65 (m, 1H), 3.59-3.51 (m, 1H), 3.20 (s, 3H), 1.77-1.53 (m, 4H), 1.50-1.41 (m, 2H), 1.38 (s, 9H).

Step 3: (1R,2S)-2-methoxy-N-methylcyclopentan-1-amine, HCl (CN-4). HCl in dioxane (474 mg, 3.25 mL, 4 molar, 25 Eq, 13.0 mmol) was added to the product from step 2 above (CN-2) (112 mg, 1 Eq, 520 μmol) in dioxane (2 mL) and the mixture stirred at rt for 16 h. The bulk solvent was removed from the reaction mixture to afford crude sub-title compound (CN-4) as a white solid. ¹H NMR (400 MHz, DMSO-d6) δ 8.61 (bs, 2H), 3.85 (td, J=4.5, 2.2 Hz, 1H), 3.49-3.31 (m, 2H), 3.29 (s, 3H), 2.01-1.90 (m, 1H), 1.89-1.79 (m, 1H), 1.76-1.49 (m, 4H).

Step 4: (1R,2S)—N-((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)-2-methoxy-N-methylcyclopentan-1-amine (CN-5). A solution of intermediate (Y-1) (31 mg, 80% Wt, 1 Eq, 53 μmol), the product from step 3 above (CN-4) (11 mg, 1.2 Eq, 64 μmol) and DIPEA (21 mg, 28 μL, 3.0 Eq, 0.16 mmol) in CHCl₃ (1 mL) was stirred 1 h at rt, then NaBH(OAc)₃ (34 mg, 3.0 Eq, 0.16 mmol) was added. The reaction mixture was stirred at 40° C. overnight. Two additional equivalents of NaBH(OAc)₃ were added. The reaction mixture was diluted with EtOAc (25 mL) and transferred into a separating funnel. The layer was washed with sat. aq. sol. of NaHCO₃ (3×5 mL) and brine (10 mL). The combined organic layers were collected, dried (MgSO₄), filtered and concentrated in vacuo. The reaction mixture was adsorbed onto Silica gel and concentrated in vacuo. The crude product was purified by chromatography on silica gel (12 g cartridge, 0-40% MeOH/DCM) to afford a 90% pure product. The 90% pure product was purified by chromatography on RP Flash C18 (12 g cartridge, 25-100% MeCN/10 mM ammonium bicarbonate) to afford the title compound (CN-5) (5 mg, 8 μmol, 20%, 96% Purity) as a pale-yellow solid. m/z 580.5 (M+H)⁺ (ES+). ¹H NMR (400 MHz, Methanol-d4) δ 8.40 (s, 1H), 8.23 (dt, J=7.8, 1.4 Hz, 1H), 8.08 (t, J=1.8 Hz, 1H), 8.03 (s, 1H), 7.84-7.77 (m, 2H), 7.64-7.53 (m, 2H), 7.48 (td, J=8.3, 7.8, 2.3 Hz, 2H), 3.97 (d, J=22.3 Hz, 2H), 3.77 (d, J=13.2 Hz, 1H), 3.39 (s, 3H), 3.27 (s, 3H), 2.74 (s, 1H), 2.28 (s, 3H), 2.02 (t, J=9.4 Hz, 2H), 1.95-1.78 (m, 2H), 1.78-1.66 (m, 2H).

### Example 160: 1-((Ethyl((2-(4′-fluoro-2′-(4-methyl-4H-1,2,4-triazol-3-yl)-[1,1′-biphenyl]-3-yl)-7-(trifluoromethyl)benzo[d]oxazol-5-yl)methyl)amino)methyl)cyclopentane-1-carbonitrile (CO-2)

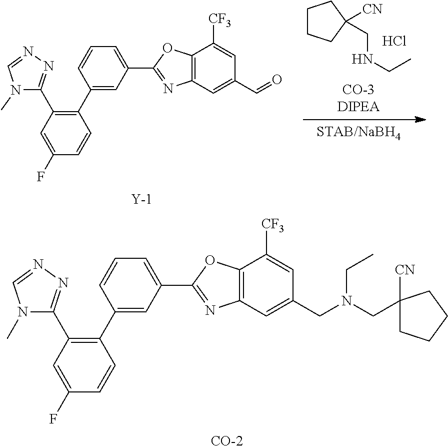

A solution of intermediate (Y-1) (31 mg, 80% Wt, 1 Eq, 53 μmol), 1-((ethylamino)methyl)cyclopentane-1-carbonitrile, HCl (CO-3) (17 mg, 2.0 Eq, 0.11 mmol) and DIPEA (21 mg, 28 μL, 3.0 Eq, 0.16 mmol) in CHCl₃ (1 mL) was stirred 1h at rt, then NaBH(OAc)₃ (34 mg, 3.0 Eq, 0.16 mmol) was added. The reaction mixture was stirred at 40° C. overnight. Two additional equivalents of NaBH(OAc)₃ were added and the mixture was stirred for additional 16 h. The reaction mixture was diluted with EtOAc (25 mL) and transferred into a separating funnel. The layer was washed with sat. aq. sol. of NaHCO₃ (2×5 mL) and brine (5 mL). The combined organic layers were collected, dried (MgSO₄), filtered and concentrated in vacuo. The reaction mixture was adsorbed onto Celite and concentrated in vacuo. The crude product was purified by chromatography on RP Flash C18 (12 g cartridge, 40-100% MeCN/10 mM ammonium bicarbonate) to afford the title compound (CO-2) (17 mg, 28 μmol, 53%, 99% Purity) as a clear white solid. m/z 603.3 (M+H)⁺ (ES+). ¹H NMR (400 MHz, DMSO-d6) δ 8.42 (s, 1H), 8.17-8.09 (m, 2H), 7.92 (t, J=1.8 Hz, 1H), 7.81 (s, 1H), 7.77 (dd, J=8.6, 5.6 Hz, 1H), 7.64-7.52 (m, 3H), 7.37 (d, J=7.7 Hz, 1H), 3.93 (s, 2H), 3.20 (s, 3H), 2.75 (s, 2H), 2.61 (q, J=7.0 Hz, 2H), 2.04-1.90 (m, 2H), 1.66 (dd, J=24.1, 11.4 Hz, 6H), 1.01 (t, J=7.0 Hz, 3H).

### Example 161: 3′-(5-((((1R,2S)-2-Hydroxycyclopentyl)amino)methyl)-7-(trifluoromethyl)benzo[d]oxazol-2-yl)-2-(4-methyl-4H-1,2,4-triazol-3-yl)-[1,1′-biphenyl]-4-carbonitrile (CP-4)

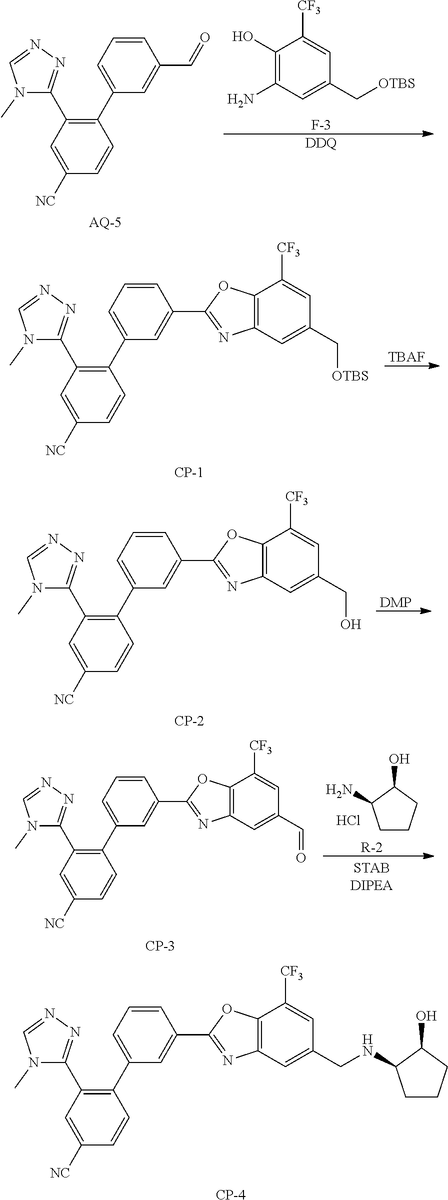

Step 1: 3′-(5-(((tert-Butyldimethylsilyl)oxy)methyl)-7-(trifluoromethyl)benzo[d]oxazol-2-yl)-2-(4-methyl-4H-1,2,4-triazol-3-yl)-[1,1′-biphenyl]-4-carbonitrile (CP-1). To a solution of intermediate (AQ-5) (106 mg, 95% Wt, 1 Eq, 349 μmol) in MeOH (1 mL) was added intermediate (F-3) (145 mg, 85% Wt, 1.1 Eq, 384 μmol). The resulting mixture was stirred at 45° C. for 12 h. After concentration in vacuo, the residue was dissolved in DCM (2 mL) and DDQ (87.2 mg, 1.1 Eq, 384 μmol) was then added. The mixture was stirred 16h at rt. Two further equivalents of DDQ were added. The resulting mixture was diluted with additional EtOAc (25 mL) and washed sequentially with saturated NaHCO₃ (2×10 mL) and brine (10 mL). The organic layer was dried (Na₂SO₄) and adsorbed onto silica gel and concentrated in vacuo. The crude product was purified by chromatography on silica gel (24 g cartridge, 0-20% MeOH/DCM) to afford the sub-title compound (CP-1) (164 mg, 0.19 mmol, 56%, 70% Purity). m/z 590.2 (M+H)⁺ (ES+).

Step 2: 3′-(5-(Hydroxymethyl)-7-(trifluoromethyl)benzo[d]oxazol-2-yl)-2-(4-methyl-4H-1,2,4-triazol-3-yl)-[1,1′-biphenyl]-4-carbonitrile (CP-2). To a solution of the product from step 1 above (CP-1) (164 mg, 70% Wt, 1 Eq, 195 μmol) in THF (2 mL) was added TBAF (76.4 mg, 292 μL, 1 molar, 1.5 Eq, 292 μmol). The mixture was stirred at rt for 2 h. The reaction mixture was diluted with EtOAc (25 mL) and transferred into a separating funnel. The layer was washed with sat. aq. sol. of NaHCO₃ (2×5 mL), sat. aq. sol. of NH₄Cl (2×5 mL) then brine. The combined organic layers were collected, dried (MgSO₄), filtered and concentrated in vacuo. The crude product was purified by chromatography on RP Flash C18 (12 g cartridge, 15-75% MeCN/10 mM ammonium bicarbonate) to afford the sub-title compound (CP-2) (51 mg, 0.11 mmol, 55%, 99% Purity) as a pale-yellow solid. m/z 476.2 (M+H)⁺ (ES+). ¹H NMR (400 MHz, Methanol-d4) δ 8.45 (s, 1H), 8.30 (d, J=7.8 Hz, 1H), 8.17 (dd, J=8.1, 1.8 Hz, 1H), 8.15-8.09 (m, 2H), 8.02 (s, 1H), 7.98 (d, J=8.1 Hz, 1H), 7.75 (s, 1H), 7.66 (t, J=7.8 Hz, 1H), 7.52 (d, J=7.7 Hz, 1H), 4.82 (s, 2H), 3.29 (s, 3H). Exchangeable proton not visible.

Step 3: 3′-(5-Formyl-7-(trifluoromethyl)benzo[d]oxazol-2-yl)-2-(4-methyl-4H-1,2,4-triazol-3-yl)-[1,1′-biphenyl]-4-carbonitrile (CP-3). The product from step 2 above (CP-2) (32 mg, 1 Eq, 67 μmol) and Dess-Martin periodinane (57 mg, 2.0 Eq, 0.13 mmol) were stirred in CHCl₃ (3 mL) at 45° C. for 5 h. The reaction mixture was diluted with EtOAc (25 mL) and transferred into a separating funnel. The layer was washed with sat. aq. sol. of NaHCO₃ (3×5 mL), sat. aq. sol. of NH₄Cl (1×5 mL) and brine. The combined organic layers were collected, dried (MgSO₄), filtered and concentrated in vacuo to afford the sub-title compound (CP-3) (32 mg, 67 μmol, 99%, 99% Purity) as a white solid. m/z 473.9 (M+H)⁺ (ES+).

Step 4: 3′-(5-((((1R,2S)-2-Hydroxycyclopentyl)amino)methyl)-7-(trifluoromethyl)benzo[d]oxazol-2-yl)-2-(4-methyl-4H-1,2,4-triazol-3-yl)-[1,1′-biphenyl]-4-carbonitrile (CP-4). A solution of the product from step 3 above (CP-3) (38 mg, 1 Eq, 80 μmol), (1S,2R)-2-aminocyclopentan-1-ol, HCl (R-2) (17 mg, 1.5 Eq, 0.12 mmol) and DIPEA (31 mg, 42 μL, 3 Eq, 0.24 mmol) in CHCl₃ (1 mL) was stirred 1 h at rt, then NaBH(OAc)₃ (51 mg, 3 Eq, 0.24 mmol) was added. The reaction mixture was stirred at 40° C. overnight. The reaction mixture was diluted with EtOAc (25 mL) and transferred into a separating funnel. The layer was washed with sat. aq. sol. of NaHCO₃ (3×5 mL) and brine (10 mL). The combined organic layers were collected, dried (MgSO₄), filtered and concentrated in vacuo. The reaction mixture was adsorbed onto silica gel and concentrated under reduced pressure. The crude product was purified by chromatography on silica gel (12 g cartridge, 0-30% MeOH/DCM) to afford the title compound (CP-4) (17 mg, 30 μmol, 38%, 99% Purity) as a white solid. m/z 559.2 (M+H)⁺ (ES+). ¹H NMR (400 MHz, Methanol-d4) δ 8.44 (s, 1H), 8.30 (dt, J=8.0, 1.2 Hz, 1H), 8.17 (dd, J=8.0, 1.7 Hz, 1H), 8.14-8.10 (m, 2H), 8.06 (s, 1H), 7.98 (d, J=8.1 Hz, 1H), 7.81 (s, 1H), 7.65 (t, J=7.8 Hz, 1H), 7.52 (dt, J=8.1, 1.3 Hz, 1H), 4.20 (td, J=4.3, 2.2 Hz, 1H), 4.07 (d, J=13.2 Hz, 1H), 3.99 (d, J=13.3 Hz, 1H), 3.29 (s, 3H), 3.01 (dt, J=12.4, 5.6 Hz, 1H), 2.02-1.70 (m, 4H), 1.59 (h, J=8.7, 8.2 Hz, 2H). Exchangeable proton not visible.

### Example 162: 3,3-Difluoro-1-((((2-(4′-fluoro-2′-(4-methyl-4H-1,2,4-triazol-3-yl)-[1,1′-biphenyl]-3-yl)-7-(trifluoromethyl)benzo[d]oxazol-5-yl)methyl)amino)methyl)cyclobutan-1-ol (CQ-1)

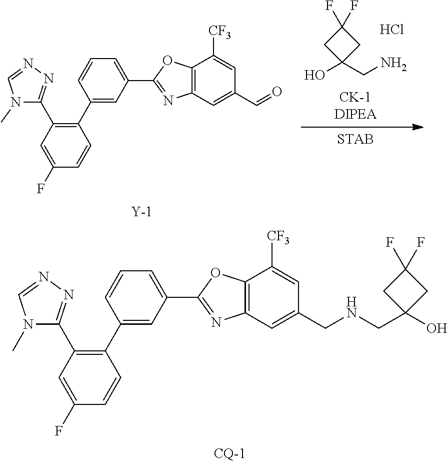

A solution of intermediate (Y-1) (30 mg, 95% Wt, 1 Eq, 61 μmol), 1-(aminomethyl)-3,3-difluorocyclobutan-1-ol, HCl (CK-1) (21 mg, 2 Eq, 0.12 mmol) and DIPEA (24 mg, 32 μL, 3.0 Eq, 0.18 mmol) in CHCl₃ (1 mL) was stirred for 1 h at rt. NaBH(OAc)₃ (39 mg, 3 Eq, 0.18 mmol) was added and the reaction mixture was stirred at rt overnight. The reaction mixture was diluted with EtOAc (10 mL) and washed with sat. aq. sol. of NaHCO₃ (2×5 mL) and brine (5 mL). The organic layer was extracted, dried (MgSO₄), filtered and concentrated in vacuo. The crude product was purified by chromatography on silica gel (12 g cartridge, 0-50% MeOH/DCM) to afford the title compound (CQ-1) (21.36 mg, 35 μmol, 57%, 95% Purity) as an off-white solid. m/z 588.1 (M+H)⁺ (ES+). ¹H NMR (400 MHz, DMSO-d6) δ 8.42 (s, 1H), 8.13 (d, J=7.8 Hz, 1H), 8.09 (s, 1H), 7.92-7.90 (m, 1H), 7.82-7.79 (m, 1H), 7.79-7.75 (m, 1H), 7.64-7.53 (m, 3H), 7.38 (d, J=7.6 Hz, 1H), 5.44 (s, 1H), 3.95 (s, 2H), 3.20 (d, 3H), 2.76-2.65 (m, 2H), 2.58 (s, 2H). CH2 masked by DMSO peak and one exchangeable proton not observed.

### Example 163: (R)-1,1,1-trifluoro-3-(((2-(4′-fluoro-2′-(4-methyl-4H-1,2,4-triazol-3-yl)-[1,1′-biphenyl]-3-yl)-7-(trifluoromethyl)benzo[d]oxazol-5-yl)methyl)amino)propan-2-ol (CR-3)

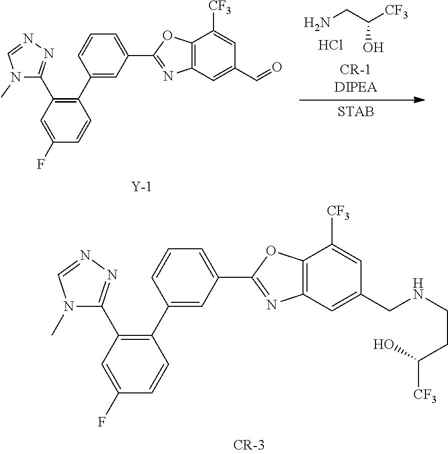

A solution of intermediate (Y-1) (30 mg, 80% Wt, 1 Eq, 51 μmol), (R)-3-amino-1,1,1-trifluoropropan-2-ol, HCl (CR-1) (17 mg, 2.0 Eq, 0.10 mmol) and DIPEA (20 mg, 27 μL, 3.0 Eq, 0.15 mmol) in CHCl₃ (1 mL) was stirred 1 h at rt, then NaBH(OAc)₃ (33 mg, 3.0 Eq, 0.15 mmol) was added. The reaction mixture was stirred at 40° C. overnight. The reaction mixture was diluted with EtOAc (25 mL) and transferred into a separating funnel. The layer was washed with sat. aq. sol. of NaHCO₃ (3×5 mL) and brine (10 mL). The combined organic layers were collected, dried (MgSO₄), filtered and concentrated in vacuo. The reaction mixture was adsorbed onto silica gel and concentrated in vacuo. The crude product was purified by chromatography on silica gel (12 g cartridge, 0-40% MeOH/DCM) to afford (R)-1,1,1-trifluoro-3-(((2-(4′-fluoro-2′-(4-methyl-4H-1,2,4-triazol-3-yl)-[1,1′-biphenyl]-3-yl)-7-(trifluoromethyl)benzo[d]oxazol-5-yl)methyl)amino)propan-2-ol at 85% purity, which was further purified by chromatography on RP Flash C18 (12 g cartridge, 40-70 MeCN/10 mM ammonium bicarbonate) to afford title compound (R)-1,1,1-trifluoro-3-(((2-(4′-fluoro-2′-(4-methyl-4H-1,2,4-triazol-3-yl)-[1,1′-biphenyl]-3-yl)-7-(trifluoromethyl)benzo[d]oxazol-5-yl)methyl)amino)propan-2-ol (CR-3) (8.0 mg, 14 μmol, 27%, 99% Purity) as a white solid. m/z 580.2 (M+H)⁺ (ES+). ¹H NMR (400 MHz, Methanol-d4) δ 8.41 (s, 1H), 8.24 (dt, J=7.9, 1.4 Hz, 1H), 8.09 (d, J=1.8 Hz, 1H), 8.04 (s, 1H), 7.84-7.76 (m, 2H), 7.64-7.53 (m, 2H), 7.53-7.43 (m, 2H), 4.15 (td, J=10.0, 5.1 Hz, 1H), 4.04 (s, 2H), 3.27 (s, 3H), 2.89 (dd, J=12.6, 3.3 Hz, 1H), 2.82 (dd, J=12.6, 9.0 Hz, 1H). Exchangeable protons not visible.

### Example 164: 1-((((2-(4′-Fluoro-2′-(4-methyl-4H-1,2,4-triazol-3-yl)-[1,1′-biphenyl]-3-yl)-7-(trifluoromethyl)-1H-benzo[d]imidazol-5-yl)methyl)amino)methyl)cyclopentane-1-carbonitrile (CS-1)

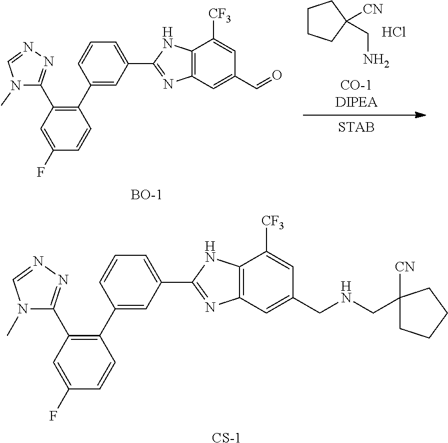

A solution of intermediate (BO-1) (23.5 mg, 95% Wt, 1 Eq, 48.0 μmol), 1-(aminomethyl)cyclopentane-1-carbonitrile, HCl (CO-1) (15.4 mg, 2 Eq, 95.9 μmol) and DIPEA (18.6 mg, 25.1 μL, 3.0 Eq, 144 μmol) in CHCl₃ (1 mL) was stirred for 1 h at rt. NaBH(OAc)₃ (30.5 mg, 3 Eq, 144 μmol) was added and the reaction mixture was stirred at rt overnight. The reaction mixture was diluted with EtOAc (10 mL) and washed with sat. aq. sol. of NaHCO₃ (2×5 mL) and brine (5 mL). The organic layer was extracted, dried (MgSO₄), filtered and concentrated in vacuo. The crude product was purified by chromatography on silica gel (12 g cartridge, 0-50% MeOH/DCM) followed by preparative HPLC (Waters, Basic (0.1% ammonium bicarbonate), Basic, Waters X-Bridge Prep-C18, 5 μm, 19×50 mm column, 0-100% MeCN in water) to afford the title compound (CS-1) (8.76 mg, 15 μmol, 31%, 98% Purity) as a pale tan solid. m/z 574.1 (M+H)⁺ (ES+); 572.3 (M−H)⁻ (ES−). ¹H NMR (400 MHz, DMSO-d6) δ 8.40 (s, 1H), 8.18-8.06 (m, 2H), 7.86-7.75 (m, 2H), 7.62 (td, J=8.6, 2.8 Hz, 1H), 7.56 (s, 1H), 7.52 (dd, J=9.2, 2.8 Hz, 1H), 7.45 (t, J=7.8 Hz, 1H), 7.08 (d, J=7.7 Hz, 1H), 3.95 (s, 2H), 3.16 (s, 3H), 2.64 (s, 2H), 2.01-1.90 (m, 2H), 1.77-1.58 (m, 6H). Two exchangeable protons not observed.

### Example 165: 4-((((2-(4′-Fluoro-2′-(4-methyl-4H-1,2,4-triazol-3-yl)-[1,1′-biphenyl]-3-yl)-7-(trifluoromethyl)-1H-benzo[d]imidazol-5-yl)methyl)amino)methyl)tetrahydro-2H-pyran-4-carbonitrile (CT-2)

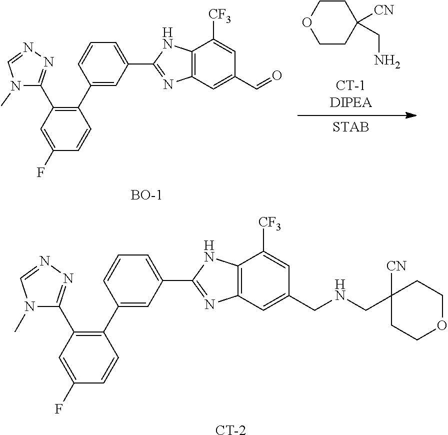 [SMILES: Cn1cnnc1-c1cc(F)ccc1-c1cccc(-c2nc3cc(C=O)cc(C(F)(F)F)c3[nH]2)c1 ; N#CC1(CN)CCOCC1 ; "BO-1" ; Cn1cnnc1-c1cc(F)ccc1-c1cccc(-c2nc3cc(CNCC4(C#N)CCOCC4)cc(C(F)(F)F)c3[nH]2)c1 ; "CT-2"]

A solution of intermediate (BO-1) (26.3 mg, 95% Wt, 1 Eq, 53.7 μmol), 4-(aminomethyl)tetrahydro-2H-pyran-4-carbonitrile (CT-1) (15.1 mg, 2 Eq, 107 μmol) and DIPEA (20.8 mg, 28.1 μL, 3.0 Eq, 161 μmol) in CHCl₃ (1 mL) was stirred for 1 h at rt. NaBH(OAc)₃ (34.1 mg, 3 Eq, 161 μmol) was added and the reaction mixture was stirred at rt overnight. The reaction mixture was diluted with EtOAc (10 mL) and washed with sat. aq. sol. of NaHCO₃ (2×5 mL) and brine (5 mL). The organic layer was extracted, dried (MgSO₄), filtered and concentrated in vacuo. The crude product was purified by chromatography on silica gel (12 g cartridge, 0-50% MeOH/DCM) followed by preparative HPLC (Waters, Basic (0.1% ammonia), Basic, Waters X-Bridge Prep-C18, 5 μm, 30×100 mm column, 0-100% MeCN in water) to afford the title compound (CT-2) (11.1 mg, 18 μmol, 34%, 98% Purity) as an off-white solid. m/z 590.1 (M+H)⁺ (ES+). ¹H NMR (400 MHz, DMSO-d6) δ 8.40 (s, 1H), 8.17-8.09 (m, 2H), 7.83-7.74 (m, 2H), 7.61 (td, J=8.6, 2.8 Hz, 1H), 7.56-7.49 (m, 2H), 7.43 (t, J=7.7 Hz, 1H), 7.06 (d, J=7.6 Hz, 1H), 3.95 (s, 2H), 3.90-3.82 (m, 2H), 3.52-3.43 (m, 2H), 3.15 (s, 3H), 2.68 (s, 2H), 1.88-1.79 (m, 2H), 1.55 (td, J=13.3, 4.4 Hz, 2H). Two exchangeable protons not observed

### Example 166: (S)-5-((((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)amino)methyl)pyrrolidin-2-one (CU-2)

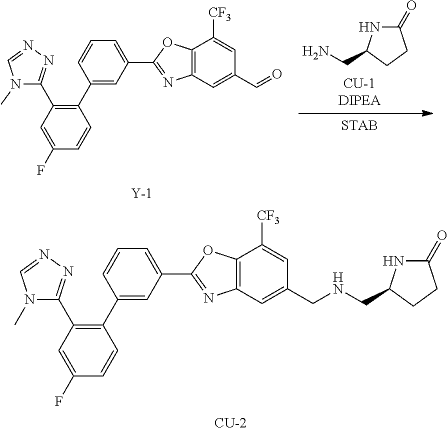

A solution of intermediate (Y-1) (30 mg, 95o Wt, 1 Eq, 61 μmol), (S)-5-(aminomethyl)pyrrolidin-2-one (CU-1) (14 mg, 2 Eq, 0.12 mmol) and DIPEA (24 mg, 32 μL, 3.0 Eq, 0.18 mmol) in CHCl₃ (1 mL) was stirred for 1 h at rt. NaBH(OAc)₃ (39 mg, 3 Eq, 0.18 mmol) was added and the reaction mixture was stirred at rt overnight. The reaction mixture was diluted with EtOAc (10 mL) and washed with sat. aq. sol. of NaHCO₃ (2×5 mL) and brine (5 mL). The organic layer was extracted, dried (MgSO₄), filtered and concentrated in vacuo. The crude product was purified by chromatography on silica gel (12 g cartridge, 0-10% MeOH/DCM) to afford the title compound (CU-2) (19.02 mg, 33 μmol, 54%, 98% Purity) as an off-white solid. m/z 565.2 (M+H)⁺ (ES+). ¹H NMR (400 MHz, DMSO-d6) δ 8.42 (s, 1H), 8.16-8.11 (m, 1H), 8.10 (s, 1H), 7.90 (t, J=1.8 Hz, 1H), 7.80-7.74 (m, 2H), 7.69 (s, 1H), 7.64-7.52 (m, 3H), 7.40-7.36 (m, 1H), 3.96-3.84 (m, 2H), 3.66-3.58 (m, 1H), 3.20 (s, 3H), 2.15-2.02 (m, 3H), 1.74-1.62 (m, 1H). CH₂ masked by DMSO peak, and one exchangeable proton not observed.

### Example 167: (1S,2S)—N-((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)-2-methoxycyclopentan-1-amine (CV-2)

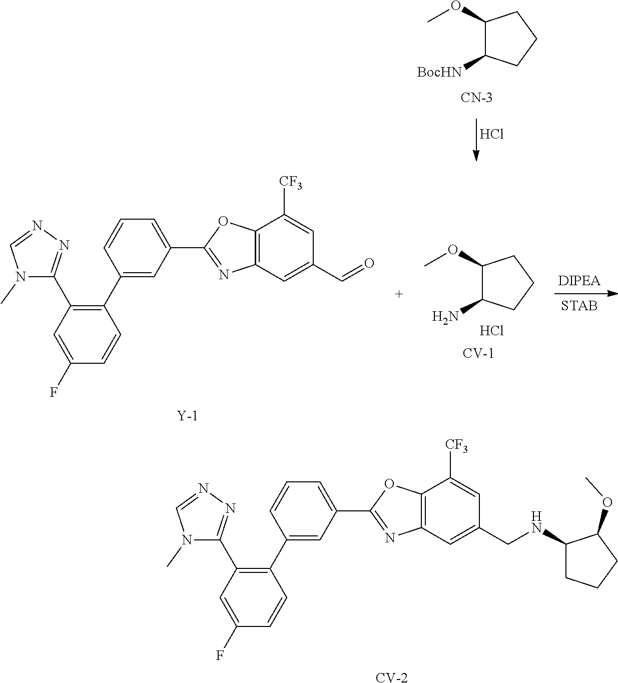

Step 1: (1R,2S)-2-Methoxycyclopentan-1-amine, HCl (CV-1). HCl (in dioxane) (0.35 g, 2.4 mL, 4 molar, 25 Eq, 9.6 mmol) was added to intermediate (CN-3) (83 mg, 1 Eq, 0.39 mmol) in dioxane (2 mL) and the mixture stirred at rt for 16 h. The bulk solvent was removed from the reaction mixture to afford crude sub-title compound (CV-1) (57 mg, 0.37 mmol, 97%, 99% Purity) as a white solid. m/z 116.3 (M+H)⁺ (ES+). ¹H NMR (400 MHz, DMSO-d6) δ 7.90 (bs, 3H), 3.78-3.72 (m, 1H), 3.44 (t, J=7.8 Hz, 1H), 3.29 (s, 3H), 1.90 (dtd, J=11.3, 7.9, 4.1 Hz, 1H), 1.80-1.66 (m, 3H), 1.64-1.47 (m, 2H).

Step 2: (1S,2S)—N-((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)-2-methoxycyclopentan-1-amine (CV-2). A solution of intermediate (Y-1) (30 mg, 95% Wt, 1 Eq, 61 μmol), the product from step 1 above (CV-1) (10 mg, 1.1 Eq, 67 μmol) and DIPEA (24 mg, 32 μL, 3.0 Eq, 0.18 mmol) in CHCl₃ (1 mL) was stirred for 1 h at rt. NaBH(OAc)₃ (39 mg, 3 Eq, 0.18 mmol) was added and the reaction mixture was stirred at rt overnight. The reaction mixture was diluted with EtOAc (10 mL) and washed with sat. aq. sol. of NaHCO₃ (2×5 mL) and brine (5 mL). The organic layer was extracted, dried (MgSO₄), filtered and concentrated in vacuo. The crude product was purified by chromatography on silica gel (12 g cartridge, 0-50% MeOH/DCM) followed by preparative HPLC (Waters, Basic (0.1% ammonia), Basic, Waters X-Bridge Prep-C18, 5 μm, 30×100 mm column, 0-100% MeCN in water) to afford the title compound (CV-2) (8.45 mg, 14 μmol, 23%, 96% Purity) as a white solid. m/z 566.0 (M+H)⁺ (ES+). ¹H NMR (400 MHz, DMSO-d6) δ 8.42 (s, 1H), 8.15-8.10 (m, 1H), 8.07 (s, 1H), 7.90 (d, J=1.8 Hz, 1H), 7.81-7.74 (m, 2H), 7.64-7.52 (m, 3H), 7.40-7.35 (m, 1H), 3.91 (s, 2H), 3.61-3.54 (m, 1H), 3.22 (s, 3H), 3.20 (s, 3H), 2.93-2.86 (m, 1H), 1.76-1.54 (m, 4H), 1.50-1.39 (m, 2H). One exchangeable proton not observed

### Example 168: (1R,2S)-2-Fluoro-N-((2-(4′-fluoro-2′-(4-methyl-4H-1,2,4-triazol-3-yl)-[1,1′-biphenyl]-3-yl)-7-(trifluoromethyl)benzo[d]oxazol-5-yl)methyl)cyclopentan-1-amine (CW-5)

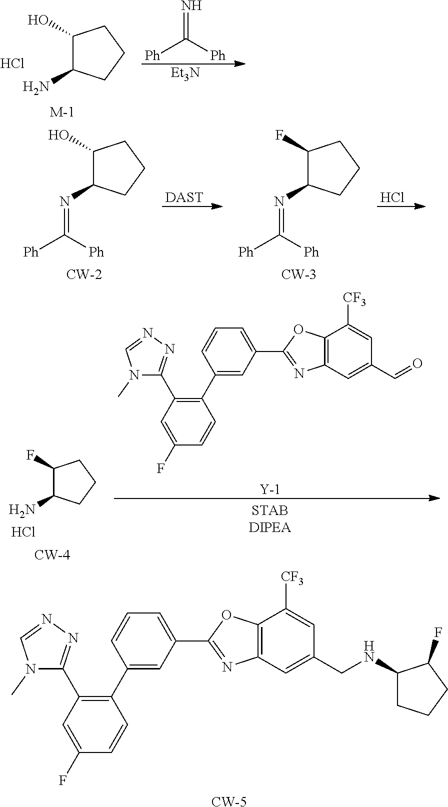

Step 1: (1R,2R)-2-((Diphenylmethylene)amino)cyclopentan-1-ol (CW-2). A solution of (1R,2R)-2-aminocyclopentan-1-ol, HCl (M-1) (500 mg, 1 Eq, 3.63 mmol), diphenylmethanimine (CW-1) (659 mg, 610 μL, 1 Eq, 3.63 mmol) and Et₃N (441 mg, 608 μL, 1.2 Eq, 4.36 mmol) in DCM (2.5 mL) was stirred at rt for 16 h. The reaction mixture was concentrated in vacuo and the crude was purified by chromatography on silica gel (40 g cartridge, 0-30% EtOAc/isohexane) to afford the sub-title compound (CW-2) (721 mg, 2.7 mmol, 74%, 99% Purity) as a colourless solid. m/z 266.2 (M+H)⁺ (ES+). ¹H NMR (400 MHz, DMSO-d6) δ 7.54-7.43 (m, 5H), 7.42-7.32 (m, 3H), 7.21-7.16 (m, 2H), 4.61 (d, J=4.6 Hz, 1H), 4.02 (p, J=5.3 Hz, 1H), 3.46 (td, J=6.5, 4.6 Hz, 1H), 1.99 (ddt, J=12.5, 8.3, 6.1 Hz, 1H), 1.77-1.40 (m, 5H).

Step 2: N-((1R,2S)-2-Fluorocyclopentyl)-1,1-diphenylmethanimine (CW-3). To a solution of the product from step 1 above (CW-2) (721 mg, 1 Eq, 2.72 mmol) in DCM (2 mL) at −20° C. was added DAST (526 mg, 431 μL, 1.2 Eq, 3.26 mmol) dropwise. The reaction mixture was allowed to reach ambient temperature and stirred for 16 h, then concentrated in vacuo. The crude product was purified by chromatography on silica gel (24 g cartridge, 0-10% EtOAc/isohexane) to afford the sub-title compound (CV-3) (531 mg, 1.9 mmol, 69%, 95% Purity) as a white solid. m/z 268.2 (M+H)⁺ (ES+). ¹H NMR (400 MHz, DMSO-d6) δ 7.60-7.46 (m, 5H), 7.46-7.33 (m, 3H), 7.23-7.16 (m, 2H), 4.99 (ddt, J=53.1, 5.8, 3.1 Hz, 1H), 3.74 (dtd, J=19.5, 6.0, 2.9 Hz, 1H), 2.24-2.04 (m, 1H), 1.90-1.75 (m, 3H), 1.75-1.52 (m, 2H).

Step 3: (1R,2S)-2-Fluorocyclopentan-1-amine, HCl (CW-4). HCl (1M in water) (0.1 g, 4 mL, 1 molar, 2 Eq, 4 mmol) was added to the product from step 2 above (CW-3) (531 mg, 1 Eq, 1.99 mmol) in hexane (4 mL) and the mixture stirred at rt for 72 h. The aqueous phase was separated and washed with hexanes. Aqueous phase was concentrated in vacuo to afford the sub-title compound (CW-4) (211 mg, 1.5 mmol, 75%, 99% Purity) as a white solid. ¹H NMR (400 MHz, DMSO-d6) δ 8.31 (s, 2H), 5.09 (ddt, J=52.2, 6.4, 3.3 Hz, 1H), 2.17-1.92 (m, 2H), 1.91-1.63 (m, 3H), 1.56 (dq, J=13.1, 7.9 Hz, 1H). 1H under water.

Step 4: (1R,2S)-2-Fluoro-N-((2-(4′-fluoro-2′-(4-methyl-4H-1,2,4-triazol-3-yl)-[1,1′-biphenyl]-3-yl)-7-(trifluoromethyl)benzo[d]oxazol-5-yl)methyl)cyclopentan-1-amine (CW-5). A solution of intermediate (Y-1) (20 mg, 95% Wt, 1 Eq, 41 μmol), the product from step 3 above (CW-4) (11 mg, 99% Wt, 2 Eq, 81 μmol) and DIPEA (16 mg, 21 μL, 3.0 Eq, 0.12 mmol) in CHCl₃ (1 mL) was stirred for 1 h at rt. NaBH(OAc)₃ (26 mg, 3.0 Eq, 0.12 mmol) was added and the reaction mixture was stirred at rt overnight. The reaction mixture was diluted with EtOAc (10 mL) and washed with sat. aq. sol. of NaHCO₃ (10 mL) and brine (10 mL). The organic layer was extracted, dried (MgSO₄), filtered and concentrated in vacuo. The crude product was purified by chromatography on silica gel (12 g cartridge, 0-10% MeOH/DCM) to afford the title compound (CW-5) (11.96 mg, 21 μmol, 50%, 95% Purity) as a flocculent white solid. m/z 554.0 (M+H)⁺ (ES+). ¹H NMR (400 MHz, DMSO-d6) δ 8.42 (s, 1H), 8.15-8.11 (m, 1H), 8.08 (s, 1H), 7.92-7.89 (m, 1H), 7.80-7.75 (m, 2H), 7.65-7.53 (m, 3H), 7.40-7.35 (m, 1H), 5.00-4.81 (m, 1H), 3.91 (s, 2H), 3.20 (s, 3H), 3.13-3.00 (m, 1H), 2.04-1.85 (m, 2H), 1.81-1.55 (m, 3H), 1.47-1.37 (m, 1H). One exchangeable proton not observed.

### Example 169: 1-((((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)amino)methyl)cyclopentane-1-carbonitrile (CX-1)

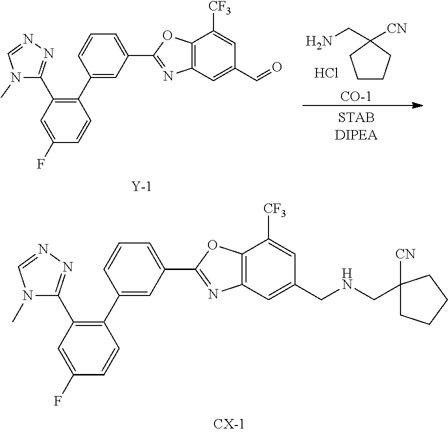

A solution of intermediate (Y-1) (30 mg, 1 Eq, 64 μmol), 1-(aminomethyl)cyclopentane-1-carbonitrile, HCl (CO-1) (21 mg, 2.0 Eq, 0.13 mmol) and DIPEA (25 mg, 34 μL, 3.0 Eq, 0.19 mmol) in CHCl₃ (1 mL) was stirred 1 h at rt, then NaBH(OAc)₃ (41 mg, 3.0 Eq, 0.19 mmol) was added. The reaction mixture was rt over the weekend. The reaction mixture was diluted with EtOAc (25 mL) and transferred into a separating funnel. The layer was washed with sat. aq. sol. of NaHCO₃ (2×5 mL) and brine (5 mL). The combined organic layers were collected, dried (MgSO₄), filtered and concentrated in vacuo. The crude product was purified by preparative HPLC (Waters, Basic (0.1% ammonia), Basic, Waters X-Bridge BEH C18 ODB Prep-C18, 5 μm, 30×100 mm column, 40-100% MeCN in water) to afford the title compound (CX-1) (7.0 mg, 12 μmol, 19%, 99% Purity) as a white solid. m/z 575.2 (M+H)⁺ (ES+). ¹H NMR (400 MHz, Methanol-d4) δ 8.41 (s, 1H), 8.22 (dt, J=7.9, 1.4 Hz, 1H), 8.09-8.01 (m, 2H), 7.83-7.76 (m, 2H), 7.63-7.52 (m, 2H), 7.49 (dd, J=8.8, 2.8 Hz, 1H), 7.45 (dt, J=7.9, 1.4 Hz, 1H), 4.06 (s, 2H), 3.27 (s, 3H), 2.78 (s, 2H), 2.15-2.07 (m, 2H), 1.88-1.70 (m, 6H). Exchangeable proton not visible.

### Example 170: (3aR,6aS)-3-((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)hexahydro-2H-cyclopenta[d]oxazol-2-one (CY-1)

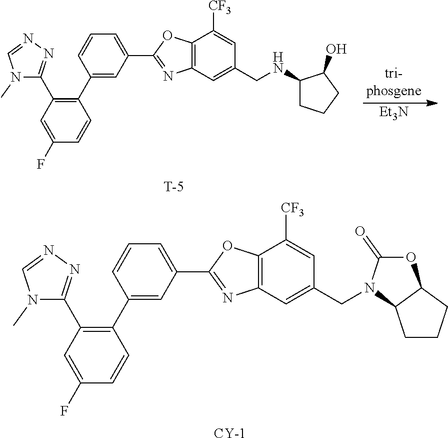

Triphosgene (4.66 mg, 0.3 Eq, 15.7 μmol) in THF (1 mL) was added dropwise to a solution of compound (T-5) (30.1 mg, 96% Wt, 1 Eq, 52.4 μmol) and Et₃N (11.7 mg, 16.1 μL, 2.2 Eq, 115 μmol) in THF (1 mL) at 0° C. under N₂. The reaction was warmed to rt and stirred for 16 h. The reaction was diluted with EtOAc (20 mL) and washed with 10% HCl solution. The organic layer was extracted, dried (MgSO₄) and concentrated in vacuo. The crude product was purified by chromatography on silica gel (12 g cartridge, 0-10% MeOH/DCM) to afford the title compound (CY-1) (7.29 mg, 12 μmol, 23%, 95% Purity) as a white solid. m/z 578.1 (M+H)⁺ (ES+). ¹H NMR (400 MHz, DMSO-d6) δ 8.42 (s, 1H), 8.17-8.11 (m, 1H), 8.06 (s, 1H), 7.90 (t, J=1.8 Hz, 1H), 7.80-7.74 (m, 1H), 7.72 (s, 1H), 7.64-7.52 (m, 3H), 7.40 (dt, J=7.9, 1.5 Hz, 1H), 4.98-4.92 (m, 1H), 4.69 (d, J=15.6 Hz, 1H), 4.44 (d, J=15.6 Hz, 1H), 4.11 (t, J=6.3 Hz, 1H), 3.21 (s, 3H), 1.90-1.77 (m, 2H), 1.72-1.58 (m, 2H), 1.52-1.36 (m, 2H).

### Example 171: 1,1,1,3,3,3-Hexafluoro-2-((((2-(4′-fluoro-2′-(4-methyl-4H-1,2,4-triazol-3-yl)-[1,1′-biphenyl]-3-yl)-7-(trifluoromethyl)benzo[d]oxazol-5-yl)methyl)amino)methyl)propan-2-ol (CZ-2)

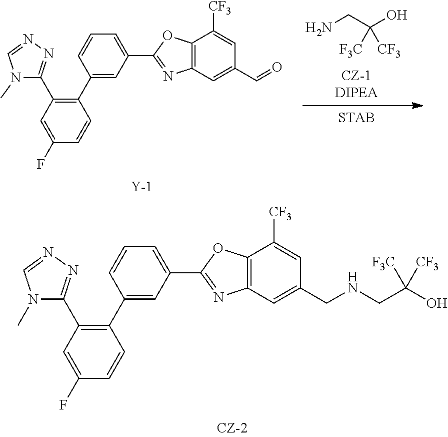

A solution of intermediate (Y-1) (30 mg, 1 Eq, 64 μmol) and 2-(aminomethyl)-1,1,1,3,3,3-hexafluoropropan-2-ol (CZ-1) (25 mg, 2.0 Eq, 0.13 mmol) in CHCl₃ (1 mL) was stirred 1 h at rt, then NaBH(OAc)₃ (41 mg, 3.0 Eq, 0.19 mmol) was added. The reaction mixture was stirred at rt 48h. The reaction mixture was diluted with EtOAc (25 mL) and transferred into a separating funnel. The layer was washed with sat. aq. sol. of NaHCO₃ (2×5 mL) and brine (5 mL). The combined organic layers were collected, dried (MgSO₄), filtered and concentrated in vacuo. The reaction mixture was adsorbed onto Celite and concentrated in vacuo. The crude product was purified by chromatography on RP Flash C18 (12 g cartridge, 50-100% MeCN/10 mM ammonium bicarbonate) to afford the title compound (CZ-2) (5 mg, 7 μmol, 10%, 97% Purity) as a clear white solid. m/z 648.2 (M+H)⁺ (ES+). ¹H NMR (400 MHz, Methanol-d4) δ 8.41 (s, 1H), 8.24 (dt, J=8.1, 1.3 Hz, 1H), 8.09 (t, J=1.8 Hz, 1H), 8.03 (s, 1H), 7.81 (dd, J=8.6, 5.4 Hz, 1H), 7.78 (s, 1H), 7.65-7.54 (m, 2H), 7.54-7.44 (m, 2H), 4.06 (s, 2H), 3.27 (s, 3H), 3.13 (s, 2H). Exchangeable protons not visible.

### Example 172: 2-Fluoro-N-((2-(4′-fluoro-2′-(4-methyl-4H-1,2,4-triazol-3-yl)-[1,1′-biphenyl]-3-yl)-7-(trifluoromethyl)benzo[d]oxazol-5-yl)methyl)-2-methylpropan-1-amine (DA-1)

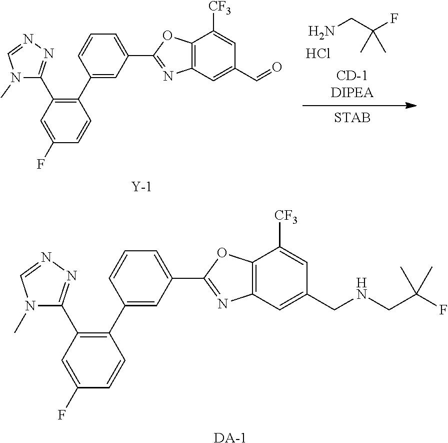

A solution of intermediate (Y-1) (30 mg, 95% Wt, 1 Eq, 61 μmol), 2-fluoro-2-methylpropan-1-amine, HCl (CD-1) (16 mg, 2 Eq, 0.12 mmol) and DIPEA (24 mg, 32 μL, 3.0 Eq, 0.18 mmol) in CHCl₃ (1 mL) was stirred for 1 h at rt. NaBH(OAc)₃ (39 mg, 3 Eq, 0.18 mmol) was added and the reaction mixture was stirred at rt overnight. Additional NaBH(OAc)₃ (39 mg, 3 Eq, 0.18 mmol) was added and the reaction was allowed to stir at rt for 3 h. The reaction mixture was diluted with EtOAc (10 mL) and washed with sat. aq. sol. of NaHCO₃ (2×5 mL) and brine (5 mL). The organic layer was extracted, dried (MgSO₄), filtered and concentrated in vacuo. The crude product was purified by chromatography on silica gel (12 g cartridge, 0-50% MeOH/DCM) to afford the title compound (DA-1) (14.08 mg, 25 μmol, 42%, 98% Purity) as a pale-yellow solid. m/z 542.2 (M+H)⁺ (ES+). ¹H NMR (400 MHz, DMSO-d6) δ 8.42 (s, 1H), 8.13 (dt, J=7.9, 1.4 Hz, 1H), 8.08 (s, 1H), 7.91 (t, J=1.8 Hz, 1H), 7.81-7.74 (m, 2H), 7.64-7.52 (m, 3H), 7.40-7.35 (m, 1H), 3.94 (s, 2H), 3.20 (s, 3H), 2.60 (d, J=19.4 Hz, 2H), 1.32 (d, J=21.6 Hz, 6H). One exchangeable proton not observed.

### Example 173: 2-(4′-Fluoro-2′-(4-methyl-4H-1,2,4-triazol-3-yl)-[1,1′-biphenyl]-3-yl)-N-((1R,2S)-2-hydroxycyclopentyl)-7-(trifluoromethyl)benzo[d]oxazole-5-carboxamide (DB-2)

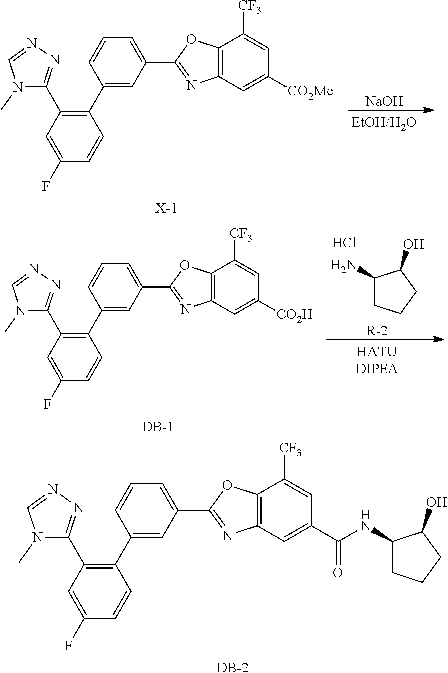

Step 1: 2-(4′-Fluoro-2′-(4-methyl-4H-1,2,4-triazol-3-yl)-[1,1′-biphenyl]-3-yl)-7-(trifluoromethyl)benzo[d]oxazole-5-carboxylic acid (DB-1). To a solution of intermediate (X-1) (35 mg, 90% Wt, 1 Eq, 63 μmol) in EtOH (1 mL)/water (1 mL) was added NaOH (3.8 mg, 48 μL, 2 molar, 1.5 Eq, 95 μmol). The mixture was stirred at rt overnight. An additional 2 Eq. NaOH 2M were added and the solution stirred at rt for 3 days. The reaction mixture was diluted with water (10 mL) and transferred into a separating funnel. The layer was extracted with EtOAc (2×5 mL). The combined aqueous layers were collected, acidified with HCl (1M aq.) and then extracted with DCM (×3). The combined organic layer was washed with brine then dried (MgSO₄). The reaction mixture was concentrated in vacuo to afford the sub-title compound (DB-1) (25 mg, 44 μmol, 69%, 85% Purity) as a pale brown solid. m/z 483.1 (M+H)⁺ (ES+). ¹H NMR (400 MHz, DMSO-d6) δ 13.64 (s, 1H), 8.59 (d, J=1.5 Hz, 1H), 8.42 (s, 1H), 8.25 (s, 1H), 8.21-8.09 (m, 1H), 7.96-7.92 (m, 1H), 7.78 (dd, J=8.6, 5.6 Hz, 1H), 7.68-7.49 (m, 3H), 7.43-7.33 (m, 1H), 3.21 (s, 3H).

Step 2: 2-(4′-Fluoro-2′-(4-methyl-4H-1,2,4-triazol-3-yl)-[1,1′-biphenyl]-3-yl)-N-((1R,2S)-2-hydroxycyclopentyl)-7-(trifluoromethyl)benzo[d]oxazole-5-carboxamide (DB-2). To a solution of the product from step 1 above (DB-1) (25 mg, 1 Eq, 52 μmol) in DMF (1 mL) were added HATU (24 mg, 1.2 Eq, 62 μmol) and DIPEA (27 mg, 36 μL, 4 Eq, 0.21 mmol). This mixture was stirred 20 min at rt then (1S,2R)-2-aminocyclopentan-1-ol, HCl (R-2) (11 mg, 1.5 Eq, 78 μmol) was added. The mixture was stirred at rt for 3 h. The reaction mixture was diluted with EtOAc (25 mL) and transferred into a separating funnel. The layer was washed with sat. aq. sol. of NH₄Cl (2×5 mL), with sat. aq. sol. of NaHCO₃ (2×5 mL) and brine. The combined organic layers were collected, dried (MgSO₄), filtered and concentrated in vacuo. The reaction mixture was adsorbed onto silica gel and concentrated in vacuo. The crude product was purified by chromatography on silica gel (12 g cartridge, 0-30% MeOH/DCM) to afford the title compound (DB-2) (17 mg, 29 μmol, 57%, 98% Purity) as a pale-yellow solid. m/z 566.2 (M+H)⁺ (ES+). ¹H NMR (400 MHz, Methanol-d4) δ 8.54 (d, J=1.6 Hz, 1H), 8.42 (s, 1H), 8.38 (d, J=7.5 Hz, 1H), 8.28 (d, J=1.4 Hz, 1H), 8.27-8.23 (m, 1H), 8.11 (t, J=1.8 Hz, 1H), 7.82 (dd, J=8.6, 5.4 Hz, 1H), 7.66-7.54 (m, 2H), 7.49 (ddd, J=6.4, 5.1, 2.2 Hz, 2H), 4.31 (td, J=4.6, 2.1 Hz, 1H), 4.29-4.17 (m, 1H), 3.28 (s, 3H), 2.12-1.84 (m, 4H), 1.83-1.75 (m, 1H), 1.69 (ddd, J=13.3, 10.0, 4.8 Hz, 1H). One exchangeable proton not visible.

### Example 174: (2-(4′-Fluoro-2′-(4-methyl-4H-1,2,4-triazol-3-yl)-[1,1′-biphenyl]-3-yl)benzo[d]oxazol-6-yl)methanol (DC-3)

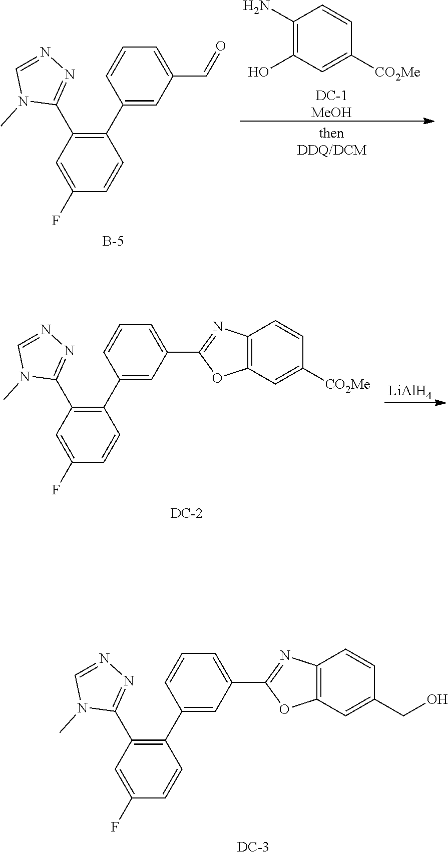

Step 1: Methyl 2-(4′-fluoro-2′-(4-methyl-4H-1,2,4-triazol-3-yl)-[1,1′-biphenyl]-3-yl)benzo[d]oxazole-6-carboxylate (DC-2). To a solution of intermediate (B-5) (161 mg, 83% Wt, 1 Eq, 475 μmol) in MeOH (2 mL) was added methyl 4-amino-3-hydroxybenzoate (DC-1) (87.4 mg, 1.1 Eq, 523 μmol). The resulting mixture was stirred at 45° C. for 12 h. After concentration in vacuo, the residue was dissolved in DCM (2 mL) and DDQ (119 mg, 1.1 Eq, 523 μmol) was then added. After stirring at rt for 1 h, the resulting mixture was diluted with additional EtOAc (25 mL) and washed sequentially with sat. NaHCO₃ (2×5 mL) and brine (5 mL). The organic layer was dried (Na₂SO₄). The crude product was purified by chromatography on silica gel (24 g cartridge, 0-30% MeOH/DCM) to afford an 80% pure product. The crude product was purified by chromatography on RP Flash C18 (12 g cartridge, 35-70% (0.1% formic acid in MeCN)/(0.1% formic acid in water)) to afford the sub-title compound (DC-2) (94 mg, 0.21 mmol, 45%, 97% Purity) as a pale brown solid. m/z 429.2 (M+H)⁺ (ES+). ¹H NMR (400 MHz, DMSO-d6) δ 8.43 (s, 1H), 8.32 (d, J=1.5 Hz, 1H), 8.17 (dt, J=7.8, 1.4 Hz, 1H), 8.05 (dd, J=8.3, 1.6 Hz, 1H), 8.00 (t, J=1.8 Hz, 1H), 7.93 (d, J=8.4 Hz, 1H), 7.78 (dd, J=8.6, 5.6 Hz, 1H), 7.65-7.51 (m, 3H), 7.34 (dt, J=7.8, 1.5 Hz, 1H), 3.91 (s, 3H), 3.17 (s, 3H).

Step 2: (2-(4′-Fluoro-2′-(4-methyl-4H-1,2,4-triazol-3-yl)-[1,1′-biphenyl]-3-yl)benzo[d]oxazol-6-yl)methanol (DC-3). To a solution of the product from step 1 above (DC-2) (91 mg, 85% Wt, 1 Eq, 0.18 mmol) in THF (10 mL) was added LiAlH₄ (14 mg, 0.36 mL, 1 molar, 2.0 Eq, 0.36 mmol) dropwise at −20° C. The mixture was stirred 1 h at that temperature. The mixture was quenched with H₂O (0.2 mL), and NaOH (aq. 15%, 0.2 mL), and H₂O (0.6 mL) was added at 0° C. MgSO₄ was added and the mixture warmed up to rt. The mixture was filtered over a silica plug and the solvent was removed in vacuo. The crude product was purified by chromatography on silica gel (24 g cartridge, 0-30% MeOH/DCM) to afford the title compound (DC-3) (64 mg, 0.16 mmol, 88%, 99% Purity). m/z 401.2 (M+H)⁺ (ES+). ¹H NMR (400 MHz, Methanol-d4) δ 8.40 (s, 1H), 8.22 (dt, J=7.8, 1.4 Hz, 1H), 8.10 (t, J=1.8 Hz, 1H), 7.81 (dd, J=8.6, 5.4 Hz, 1H), 7.75-7.69 (m, 2H), 7.61-7.54 (m, 2H), 7.49 (dd, J=8.8, 2.7 Hz, 1H), 7.46-7.40 (m, 2H), 4.79 (s, 2H), 3.21 (s, 3H). Exchangeable proton not visible.

### Example 175: (1S,2R)-2-(((2-(4′-Fluoro-2′-(4-methyl-4H-1,2,4-triazol-3-yl)-[1,1′-biphenyl]-3-yl)benzo[d]oxazol-6-yl)methyl)amino)cyclopentan-1-ol (DD-2)

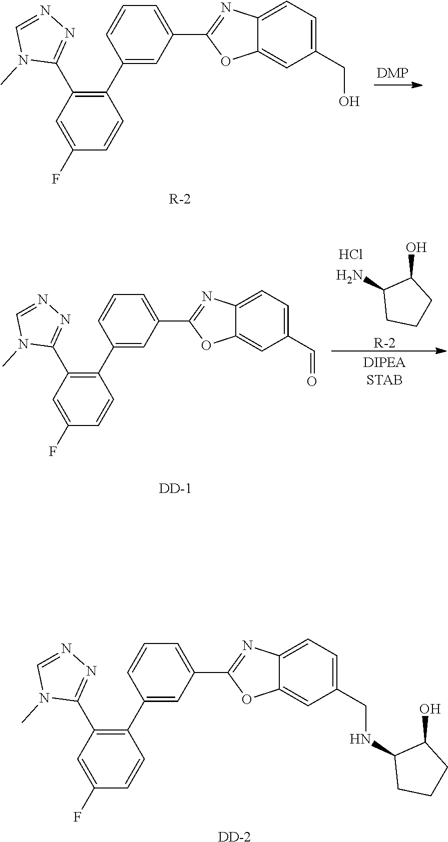

Step 1: 2-(4′-Fluoro-2′-(4-methyl-4H-1,2,4-triazol-3-yl)-[1,1′-biphenyl]-3-yl)benzo[d]oxazole-6-carbaldehyde (DD-1). Compound (DC-3) (54.0 mg, 99% Wt, 1 Eq, 134 μmol) and Dess-Martin Periodinane (113 mg, 2.0 Eq, 267 μmol) were stirred in CHCl₃ (5 mL) at 40° C. for 2 h. The reaction mixture was diluted with EtOAc (20 mL) and washed with sat. aq. NaHCO₃ (3×10 mL) and brine (10 mL). Organics were separated, dried (MgSO₄), filtered and evaporated, affording the sub-title compound (DD-1) (54 mg, 0.13 mmol, 100%, 99% Purity). m/z 399.2 (M+H)⁺ (ES+)

Step 2: (1S,2R)-2-(((2-(4′-Fluoro-2′-(4-methyl-4H-1,2,4-triazol-3-yl)-[1,1′-biphenyl]-3-yl)benzo[d]oxazol-6-yl)methyl)amino)cyclopentan-1-ol (DD-2). A solution of the product from step 1 above (DD-1) (54.0 mg, 91% Wt, 1 Eq, 123 μmol), (1S,2R)-2-aminocyclopentan-1-ol, HCl (R-2) (33.9 mg, 2.0 Eq, 247 μmol) and DIPEA (63.8 mg, 85.9 μL, 4.0 Eq, 493 μmol) in CHCl₃ (1 mL) was stirred 1 h at rt, then NaBH(OAc)₃ (131 mg, 5.0 Eq, 617 μmol) was added. The reaction mixture was stirred at rt overnight. The reaction mixture was diluted with EtOAc (25 mL) and transferred into a separating funnel. The layer was washed with sat. aq. sol. of NaHCO₃ (2×10 mL) and brine. The combined organic layers were collected, dried (MgSO₄), filtered and concentrated in vacuo. The crude product was purified by chromatography on silica gel (12 g cartridge, 0-20% (0.7 M ammonia/MeOH)/DCM) to afford the title compound (DD-2) (42 mg, 86 μmol, 70%, 99% Purity) as a clear white solid. m/z 484.2 (M+H)⁺ (ES+). ¹H NMR (400 MHz, Methanol-d4) δ 8.40 (s, 1H), 8.21 (dt, J=7.9, 1.4 Hz, 1H), 8.09 (t, J=1.8 Hz, 1H), 7.80 (dd, J=8.6, 5.5 Hz, 1H), 7.74 (d, J=1.5 Hz, 1H), 7.71 (d, J=8.2 Hz, 1H), 7.61-7.53 (m, 2H), 7.47 (ddd, J=9.9, 8.5, 2.1 Hz, 2H), 7.41 (ddd, J=7.8, 1.9, 1.2 Hz, 1H), 4.18 (td, J=4.3, 2.2 Hz, 1H), 4.03 (d, J=13.1 Hz, 1H), 3.95 (d, J=13.1 Hz, 1H), 3.21 (s, 3H), 3.00 (ddd, J=9.3, 7.6, 4.3 Hz, 1H), 2.04-1.68 (m, 4H), 1.66-1.48 (m, 2H). Exchangeable protons not visible.

### Example 176: 1-(2-(4′-Fluoro-2′-(4-methyl-4H-1,2,4-triazol-3-yl)-[1,1′-biphenyl]-3-yl)-7-(trifluoromethyl)benzo[d]oxazol-5-yl)-N-((1-methoxycyclobutyl)methyl)methanamine (DE-2)

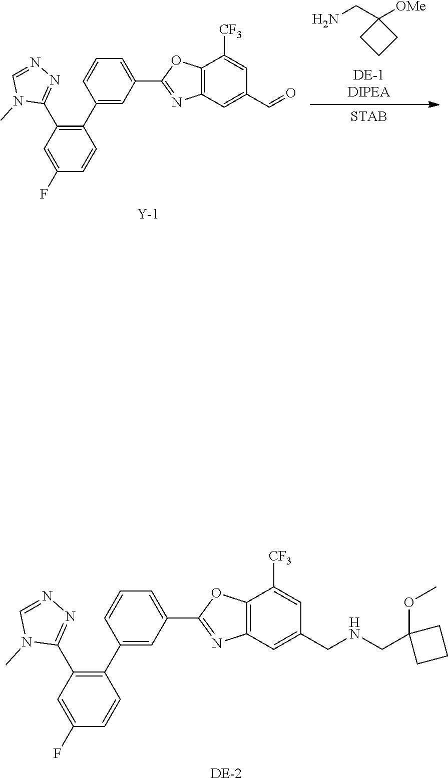

A solution of intermediate (Y-1) (30 mg, 85% Wt, 1 Eq, 55 μmol), (1-methoxycyclobutyl)methanamine (DE-1) (13 mg, 2 Eq, 0.11 mmol) and DIPEA (21 mg, 29 μL, 3.0 Eq, 0.16 mmol) in CHCl₃ (1 mL) was stirred for 1 h at rt. NaBH(OAc)₃ (35 mg, 3.0 Eq, 0.16 mmol) was added and the reaction mixture was stirred at rt overnight. The reaction mixture was diluted with EtOAc (10 mL) and washed with sat. aq. sol. of NaHCO₃ (10 mL) and brine (10 mL). The organic layer was extracted, dried (MgSO₄), filtered and concentrated in vacuo. The crude product was purified by chromatography on silica gel (12 g cartridge, 0-10% MeOH/DCM) to afford the title compound (DE-2) (11.47 mg, 20 μmol, 36%, 98% Purity) as an off-white solid. m/z 566.1 (M+H)⁺ (ES+). ¹H NMR (400 MHz, DMSO-d6) δ 8.42 (s, 1H), 8.13 (dt, J=7.9, 1.4 Hz, 1H), 8.08 (s, 1H), 7.91 (t, J=1.8 Hz, 1H), 7.82 (s, 1H), 7.77 (dd, J=8.6, 5.6 Hz, 1H), 7.64-7.52 (m, 3H), 7.37 (dt, J=8.0, 1.4 Hz, 1H), 3.95 (s, 2H), 3.20 (s, 3H), 3.02 (s, 3H), 2.62 (s, 2H), 2.05-1.93 (m, 2H), 1.91-1.80 (m, 2H), 1.70-1.59 (m, 1H), 1.54-1.40 (m, 1H). One exchangeable proton not observed.

### Example 177: (1S,2R)-2-(((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)amino)cyclohexan-1-ol (DF-2)

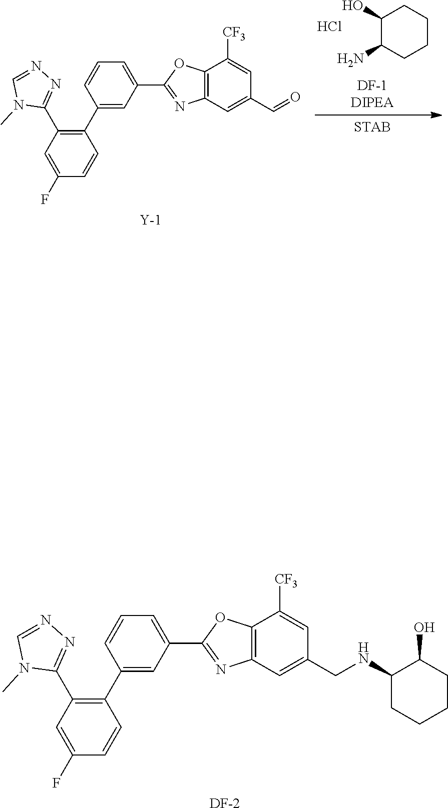

A solution of intermediate (Y-1) (30 mg, 85% Wt, 1 Eq, 55 μmol), (1S,2R)-2-aminocyclohexan-1-ol, HCl (DF-1) (17 mg, 2 Eq, 0.11 mmol) and DIPEA (21 mg, 29 μL, 3.0 Eq, 0.16 mmol) in CHCl₃ (1 mL) was stirred for 1 h at rt. NaBH(OAc)₃ (35 mg, 3.0 Eq, 0.16 mmol) was added and the reaction mixture was stirred at rt overnight. The reaction mixture was diluted with EtOAc (10 mL) and washed with sat. aq. sol. of NaHCO₃ (10 mL) and brine (10 mL). The organic layer was extracted, dried (MgSO₄), filtered and concentrated in vacuo. The crude product was purified by chromatography on silica gel (12 g cartridge, 0-20% MeOH/DCM) to afford the title compound (DF-2) (10.43 mg, 18 μmol, 33%, 97% Purity) as an off-white solid. m/z 566.3 (M+H)⁺ (ES+). ¹H NMR (400 MHz, DMSO-d6) δ 8.42 (s, 1H), 8.13 (dt, J=7.9, 1.4 Hz, 1H), 8.09 (s, 1H), 7.90 (t, J=1.8 Hz, 1H), 7.82-7.74 (m, 2H), 7.65-7.52 (m, 3H), 7.38 (dt, J=7.8, 1.4 Hz, 1H), 4.34 (s, 1H), 3.93 (s, 2H), 3.79 (s, 1H), 3.20 (s, 3H), 1.75-1.65 (m, 1H), 1.64-1.39 (m, 4H), 1.38-1.11 (m, 3H). Two exchangeable protons not observed.

### Example 178: (S)-1-(((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)amino)butan-2-ol (DG-2)

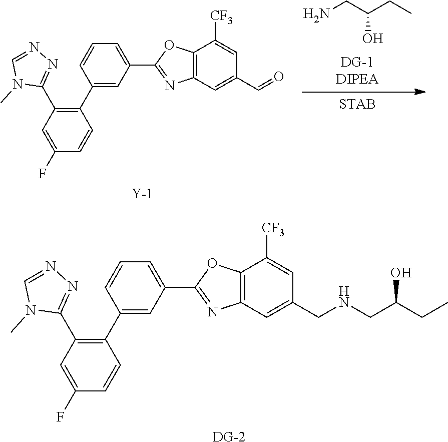

A solution of intermediate (Y-1) (30 mg, 85% Wt, 1 Eq, 55 μmol), (S)-1-aminobutan-2-ol (DG-1) (9.7 mg, 2 Eq, 0.11 mmol) and DIPEA (21 mg, 29 μL, 3.0 Eq, 0.16 mmol) in CHCl₃ (1 mL) was stirred for 1 h at rt. NaBH(OAc)₃ (35 mg, 3.0 Eq, 0.16 mmol) was added and the reaction mixture was stirred at rt overnight. The reaction mixture was diluted with EtOAc (10 mL) and washed with sat. aq. sol. of NaHCO₃ (10 mL) and brine (10 mL). The organic layer was extracted, dried (MgSO₄), filtered and concentrated in vacuo. The crude product was purified by chromatography on silica gel (12 g cartridge, 0-60% MeOH/DCM) to afford the title compound (DG-2) (11.58 mg, 21 μmol, 38%, 98% Purity) as a pale tan solid. m/z 540.6 (M+H)⁺ (ES+). ¹H NMR (400 MHz, DMSO-d6) δ 8.42 (s, 1H), 8.13 (dt, J=7.9, 1.4 Hz, 1H), 8.08 (s, 1H), 7.91 (t, J=1.8 Hz, 1H), 7.80-7.74 (m, 2H), 7.64-7.52 (m, 3H), 7.38 (dt, J=7.8, 1.5 Hz, 1H), 4.50 (s, 1H), 3.93 (s, 2H), 3.52-3.42 (m, 1H), 3.20 (s, 3H), 2.47-2.40 (m, 2H), 1.50-1.23 (m, 2H), 0.85 (t, J=7.4 Hz, 3H). One exchangeable proton not observed.

### Example 179: (3R,4R)-4-(((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)amino)tetrahydrofuran-3-ol (DH-2)

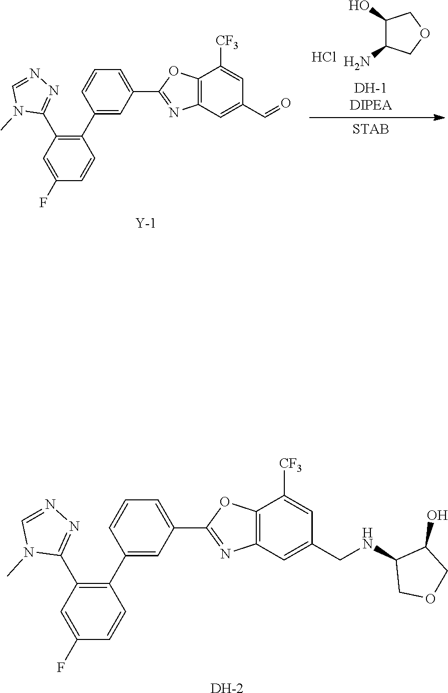

A solution of intermediate (Y-1) (30 mg, 85% Wt, 1 Eq, 55 μmol), (3R,4R)-4-aminotetrahydrofuran-3-ol, HCl (DH-1) (15 mg, 2 Eq, 0.11 mmol) and DIPEA (21 mg, 29 μL, 3.0 Eq, 0.16 mmol) in CHCl₃ (1 mL) was stirred for 1 h at rt. NaBH(OAc)₃ (35 mg, 3.0 Eq, 0.16 mmol) was added and the reaction mixture was stirred at rt overnight. Additional NaBH(OAc)₃ (35 mg, 3.0 Eq, 0.16 mmol) was added and the reaction mixture was stirred at rt for 3 h. The reaction mixture was diluted with EtOAc (10 mL) and washed with sat. aq. sol. of NaHCO₃ (10 mL) and brine (10 mL). The organic layer was extracted, dried (MgSO₄), filtered and concentrated in vacuo. The crude product was purified by chromatography on silica gel (12 g cartridge, 0-10% MeOH/DCM) to afford the title compound (DH-2) (8.96 mg, 16 μmol, 29%, 97% Purity) as a white solid. m/z 554.3 (M+H)⁺ (ES+). ¹H NMR (400 MHz, DMSO-d6) δ 8.42 (s, 1H), 8.15-8.08 (m, 2H), 7.91 (t, J=1.8 Hz, 1H), 7.81-7.74 (m, 2H), 7.64-7.52 (m, 3H), 7.38 (dt, J=7.9, 1.4 Hz, 1H), 4.14-4.10 (m, 1H), 3.99 (d, J=13.9 Hz, 1H), 3.88 (d, J=13.9 Hz, 1H), 3.83-3.74 (m, 2H), 3.61 (dd, J=9.4, 2.0 Hz, 1H), 3.23-3.19 (m, 3H), 3.19-3.11 (m, 1H). One proton masked by water peak and two exchangeable protons not observed.

### Example 180: 2-(4′-Fluoro-2′-(4-methyl-4H-1,2,4-triazol-3-yl)-[1,1′-biphenyl]-3-yl)-N-hydroxy-7-(trifluoromethyl)benzo[d]oxazole-5-carboxamide (DI-1)

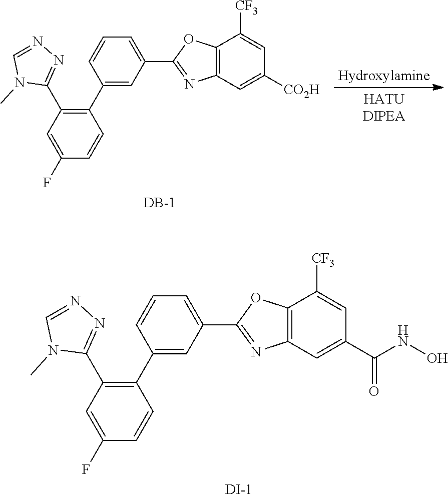 [SMILES: Cn1cnnc1-c1cc(F)ccc1-c1cccc(-c2nc3cc(C(=O)O)cc(C(F)(F)F)c3o2)c1 ; "Hydroxylamine" ; "DB-1" ; Cn1cnnc1-c1cc(F)ccc1-c1cccc(-c2nc3cc(C(=O)NO)cc(C(F)(F)F)c3o2)c1 ; "DI-1"]

To a solution of intermediate (DB-1) (30 mg, 1 Eq, 62 μmol) in DMF (1 mL) were added HATU (35 mg, 1.5 Eq, 93 μmol) and DIPEA (40 mg, 54 μL, 5 Eq, 0.31 mmol). This mixture was stirred 20 min at rt then hydroxylamine, HCl (8.6 mg, 2.0 Eq, 0.12 mmol) was added. The mixture was stirred at rt for 36 h. The reaction mixture was diluted with EtOAc (25 mL) and transferred into a separating funnel. The layer was washed with sat. aq. sol. of NH₄Cl (2×5 mL), with sat. aq. sol. of NaHCO₃ (2×5 mL) and brine. The combined organic layers were collected, dried MgSO₄, filtered and concentrated in vacuo. The reaction mixture was adsorbed onto Celite and concentrated in vacuo. The crude product was purified by chromatography on RP Flash C18 (12 g cartridge, 30-65% (0.1% formic acid in MeCN)/(0.1% formic acid in Water)) to afford the title compound (DI-1) (6 mg, 0.01 mmol, 20%, 95% Purity) as a clear grey solid. m/z 498.4 (M+H)⁺ (ES+). ¹H NMR (400 MHz, DMSO-d6) δ 8.47 (s, 1H), 8.42 (s, 1H), 8.15 (d, J=8.2 Hz, 2H), 7.93 (t, J=1.8 Hz, 1H), 7.78 (dd, J=8.6, 5.7 Hz, 1H), 7.58 (ddq, J=17.3, 8.9, 2.7 Hz, 3H), 7.40 (d, J=7.7 Hz, 1H), 3.21 (s, 3H). Exchangeable protons not visible.

### Example 181: 2-(4′-Fluoro-2′-(4-methyl-4H-1,2,4-triazol-3-yl)-[1,1′-biphenyl]-3-yl)-N-methoxy-N-methyl-7-(trifluoromethyl)benzo[d]oxazole-5-carboxamide (DJ-2)

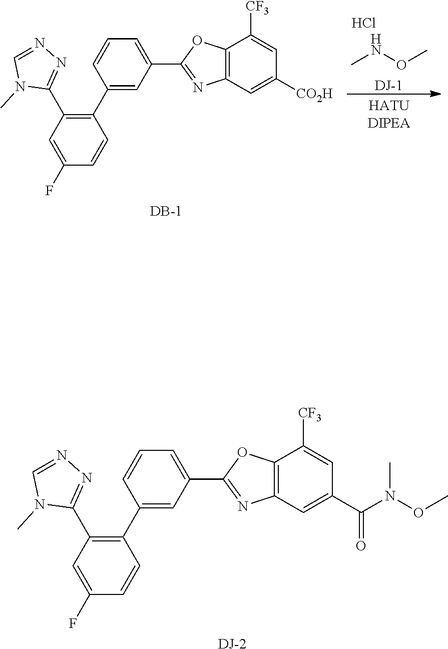

To a solution of intermediate (DB-1) (140 mg, 1 Eq, 290 μmol) in DMF (10 mL) were added HATU (166 mg, 1.5 Eq, 435 μmol) and DIPEA (188 mg, 253 μL, 5 Eq, 1.45 mmol). This mixture was stirred 20 min at rt then N,O-dimethylhydroxylamine, HCl (DJ-1) (56.6 mg, 2.0 Eq, 580 μmol) was added. The mixture was stirred at rt over the weekend. The reaction mixture was diluted with EtOAc (25 mL) and transferred into a separating funnel. The layer was washed with sat. aq. sol. of NH₄Cl (2×5 mL), with sat. aq. sol. of NaHCO₃ (2×5 mL) and brine. The combined organic layers were collected, dried (MgSO₄), filtered and concentrated in vacuo. The reaction mixture was adsorbed onto silica gel and concentrated in vacuo. The crude product was purified by chromatography on silica gel (12 g cartridge, 0-10% MeOH/DCM) to afford the title compound (DJ-2) (121 mg, 0.23 mmol, 78%, 98% Purity) as a pale-yellow solid. m/z 526.1 (M+H)⁺ (ES+). ¹H NMR (400 MHz, DMSO-d6) δ 8.42 (s, 1H), 8.36 (d, J=1.5 Hz, 1H), 8.16 (dt, J=7.9, 1.3 Hz, 1H), 7.98 (d, J=1.5 Hz, 1H), 7.94 (t, J=1.8 Hz, 1H), 7.78 (dd, J=8.6, 5.7 Hz, 1H), 7.65-7.51 (m, 3H), 7.40 (dt, J=8.1, 1.3 Hz, 1H), 3.58 (s, 3H), 3.33 (s, 3H), 3.21 (s, 3H).

### Example 182: 1-(2-(4′-Fluoro-2′-(4-methyl-4H-1,2,4-triazol-3-yl)-[1,1′-biphenyl]-3-yl)-7-(trifluoromethyl)benzo[d]oxazol-5-yl)ethan-1-one (DK-1)

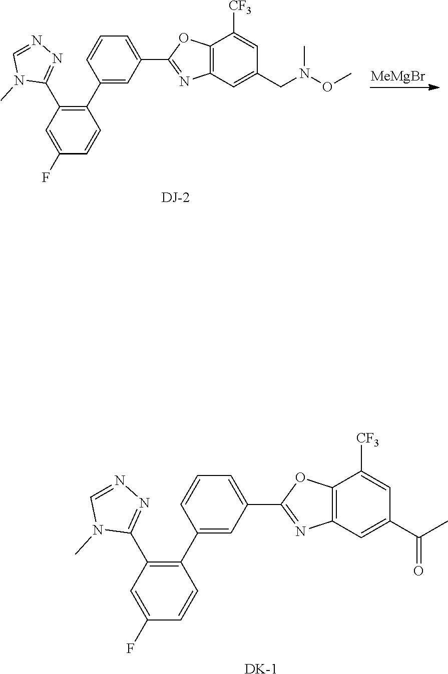

To a solution of compound (DJ-2) (100 mg, 1 Eq, 190 μmol) in THF (2 mL) was added methylmagnesium bromide (34.0 mg, 95.2 μL, 3 molar, 1.5 Eq, 285 μmol) dropwise at −78° C. This mixture was stirred 1 h at this temperature. The mixture was then warmed up to rt for 2 h. The mixture was quenched with sat. aq. sol. of NH₄Cl (2 mL) and stirred 5 min at rt. The reaction mixture was diluted with EtOAc (10 mL) and transferred into a separating funnel. The layer was washed with sat. aq. sol. of NH₄Cl (2×5 mL), with sat. aq. sol. of NaHCO₃ (2×5 mL) and brine. The combined organic layers were collected, dried (MgSO₄), filtered and concentrated in vacuo to afford the title compound (DK-1) (85 mg, 0.17 mmol, 88%, 95% Purity) as a brown solid. m/z 481.2 (M+H)⁺ (ES+). ¹H NMR (400 MHz, Methanol-d4) δ 8.68-8.64 (m, 1H), 8.42 (s, 1H), 8.35 (dd, J=1.7, 0.8 Hz, 1H), 8.27 (dt, J=8.0, 1.3 Hz, 1H), 8.11 (t, J=1.7 Hz, 1H), 7.82 (dd, J=8.7, 5.4 Hz, 1H), 7.66-7.55 (m, 2H), 7.55-7.48 (m, 2H), 3.28 (s, 3H), 2.77 (s, 3H).

### Example 183: 2-(4′-Fluoro-2′-(4-methyl-4H-1,2,4-triazol-3-yl)-[1,1′-biphenyl]-3-yl)-7-(trifluoromethyl)-5-vinylbenzo[d]oxazole (DL-1)

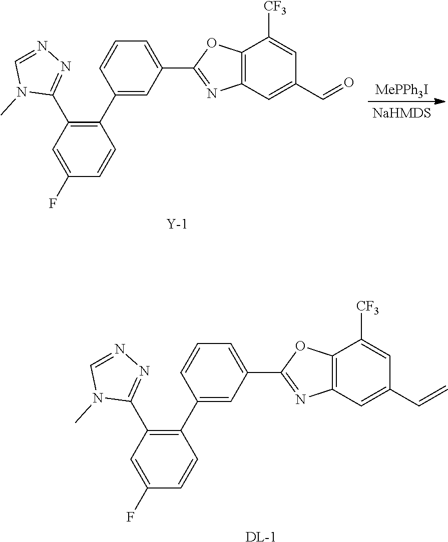

NaHMDS (2M in THF) (59.0 mg, 161 μL, 2 molar, 1.5 Eq, 322 μmol) was added to a solution of MePPh₃I (130 mg, 1.5 Eq, 322 μmol) in THF (2 mL) and the mixture stirred at rt for 1 h. The reaction was cooled to −40° C. and intermediate (Y-1) (100 mg, 1.0 Eq, 214 μmol) in THF (2 mL) added. The mixture was stirred 1 h at −40° C., then allowed to reach rt overnight. HCl 1M (10 mL) was added and the mixture partitioned between EtOAc (25 mL) and water (20 mL). The organic layer was washed with HCl 1M (3×5 mL) and brine. The combined organic layers were collected, dried (MgSO₄), filtered and concentrated in vacuo. The crude product was purified by chromatography on silica gel (40 g cartridge, 60-100% EtOAc/isohexane) to afford a 50% pure product. The 50% pure product was purified by chromatography on RP Flash C18 (12 g cartridge, 50-85% (0.1% formic acid in MeCN)/(0.1% formic acid in water)) to afford the title compound (DL-1) (45 mg, 96 μmol, 45%, 99% Purity) as a pale yellow solid. m/z 465.3 (M+H)⁺ (ES+). ¹H NMR (400 MHz, Methanol-d4) δ 8.41 (s, 1H), 8.24 (dt, J=7.9, 1.4 Hz, 1H), 8.09 (d, J=1.6 Hz, 2H), 7.85-7.77 (m, 2H), 7.66-7.53 (m, 2H), 7.53-7.42 (m, 2H), 6.96 (dd, J=17.6, 11.0 Hz, 1H), 5.98 (d, J=17.5 Hz, 1H), 5.44 (d, J=11.0 Hz, 1H), 3.27 (s, 3H).

### Example 184: (S)-1,1,1-Trifluoro-3-(((2-(4′-fluoro-2′-(4-methyl-4H-1,2,4-triazol-3-yl)-[1,1′-biphenyl]-3-yl)-7-(trifluoromethyl)benzo[d]oxazol-5-yl)methyl)amino)propan-2-ol (DM-2)

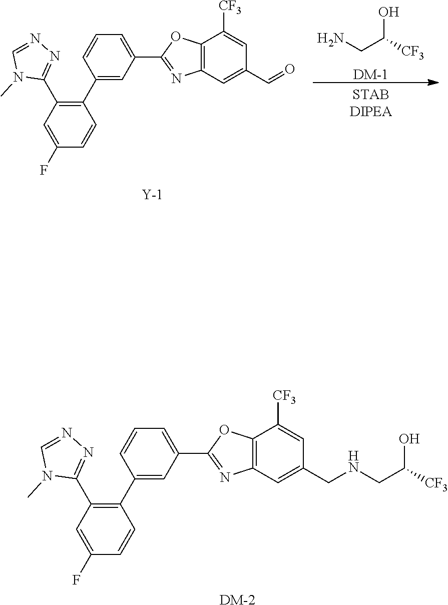

A solution of intermediate (Y-1) (30 mg, 85% Wt, 1 Eq, 55 μmol), (S)-3-amino-1,1,1-trifluoropropan-2-ol (10 mg, 1.4 Eq, 77 μmol) and DIPEA (21 mg, 29 μL, 3.0 Eq, 0.16 mmol) in CHCl₃ (1 mL) was stirred for 1 h at rt. NaBH(OAc)₃ (35 mg, 3.0 Eq, 0.16 mmol) was added and the reaction mixture was stirred at rt overnight. The reaction mixture was diluted with EtOAc (10 mL) and washed with sat. aq. sol. of NaHCO₃ (10 mL) and brine (10 mL). The organic layer was extracted, dried (MgSO₄), filtered and concentrated in vacuo. The crude product was purified by chromatography on silica gel (12 g cartridge, 0-10% MeOH/DCM) to afford the title compound (DM-2) (13.03 mg, 22 μmol, 39%, 96% Purity) as a pale tan solid. m/z 580.1 (M+H)⁺ (ES+). ¹H NMR (400 MHz, DMSO-d6) δ 8.42 (s, 1H), 8.13 (dt, J=7.9, 1.3 Hz, 1H), 8.09 (s, 1H), 7.91 (t, J=1.8 Hz, 1H), 7.81-7.74 (m, 2H), 7.64-7.52 (m, 3H), 7.38 (dt, J=7.9, 1.3 Hz, 1H), 6.26 (d, J=6.4 Hz, 1H), 4.14-4.04 (m, 1H), 3.94 (s, 2H), 3.19 (s, 3H), 2.76-2.69 (m, 1H), 2.69-2.61 (m, 1H). One exchangeable proton not observed.

### Example 185: 3-(((2-(4′-fluoro-2′-(4-methyl-4H-1,2,4-triazol-3-yl)-[1,1′-biphenyl]-3-yl)-7-(trifluoromethyl)benzo[d]oxazol-5-yl)methyl)amino)-2,2-dimethylpropanenitrile (DN-2)

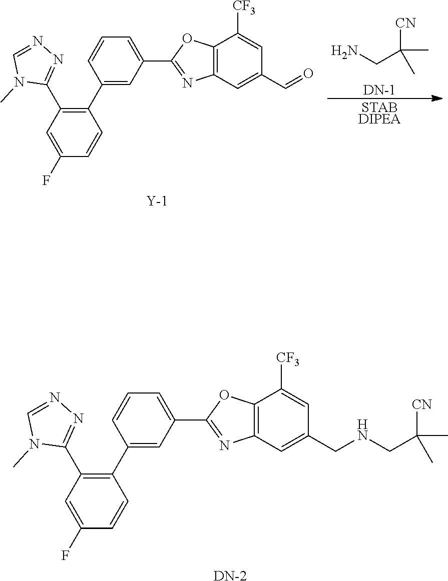

A solution of intermediate (Y-1) (30 mg, 85% Wt, 1 Eq, 55 μmol), 3-amino-2,2-dimethylpropanenitrile (DN-1) (11 mg, 2 Eq, 0.11 mmol) and DIPEA (21 mg, 29 μL, 3.0 Eq, 0.16 mmol) in CHCl₃ (1 mL) was stirred for 1 h at rt. NaBH(OAc)₃ (35 mg, 3.0 Eq, 0.16 mmol) was added and the reaction mixture was stirred at rt overnight. Additional NaBH(OAc)₃ (35 mg, 3.0 Eq, 0.16 mmol) was added and the reaction mixture was stirred at rt for 3 h. The reaction mixture was diluted with EtOAc (10 mL) and washed with sat. aq. sol. of NaHCO₃ (10 mL) and brine (10 mL). The organic layer was extracted, dried (MgSO₄), filtered and concentrated in vacuo. The crude product was purified by chromatography on silica gel (12 g cartridge, 0-10% MeOH/DCM). Product containing fractions were combined and concentrated in vacuo. The crude was dissolved in MeOH (2 mL) and loaded onto SCX (ca. 0.5 g) and washed with MeOH (5 mL), the product was then eluted with 0.7 M NH₃/MeOH (5 mL) and concentrated in vacuo to afford the title compound (DN-2) (13.43 mg, 23 μmol, 43%, 95% Purity) as a yellow solid. m/z 549.2 (M+H)⁺ (ES+). ¹H NMR (400 MHz, DMSO-d6) δ 8.42 (s, 1H), 8.16-8.09 (m, 2H), 7.91 (t, J=1.8 Hz, 1H), 7.83 (s, 1H), 7.78 (dd, J=8.6, 5.7 Hz, 1H), 7.64-7.52 (m, 3H), 7.38 (dt, J=7.9, 1.4 Hz, 1H), 3.97 (s, 2H), 3.22 (s, 3H), 2.57 (s, 2H), 1.27 (s, 6H), 1.25-1.22 (m, 1H).

### Example 186: (R)-2-(Azetidin-1-yl)-2-(2-(4′-fluoro-2′-(4-methyl-4H-1,2,4-triazol-3-yl)-[1,1′-biphenyl]-3-yl)-7-(trifluoromethyl)benzo[d]oxazol-5-yl)ethan-1-ol (DO-6) and (S)-2-(Azetidin-l-yl)-2-(2-(4′-fluoro-2′-(4-methyl-4H-1,2,4-triazol-3-yl)-[1,1′-biphenyl]-3-yl)-7-(trifluoromethyl)benzo[d]oxazol-5-yl)ethan-1-ol (DO-7)

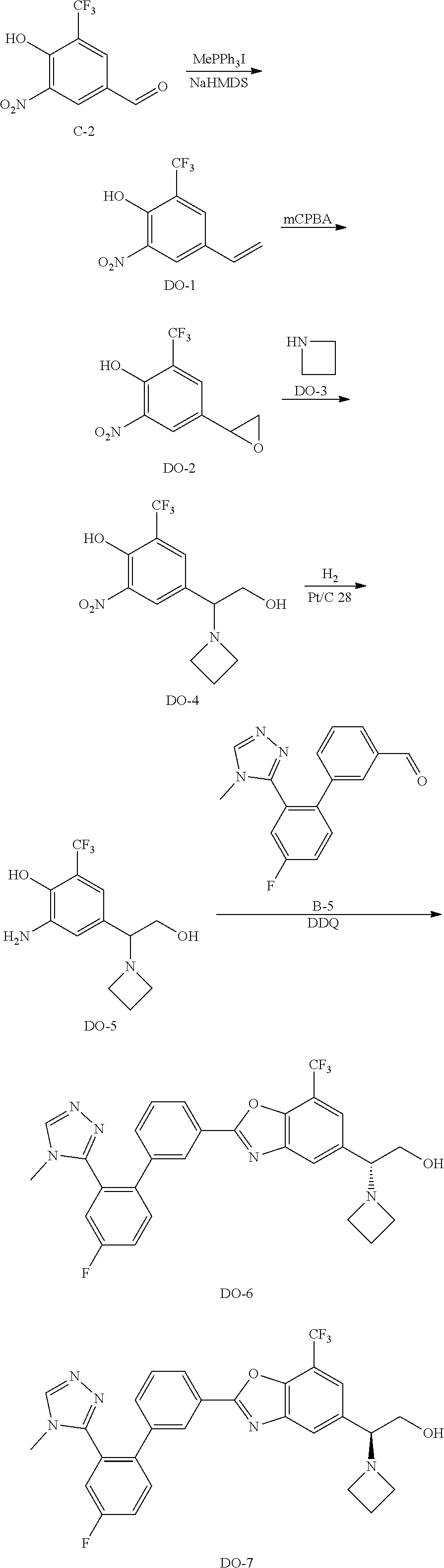

Step 1: 2-Nitro-6-(trifluoromethyl)-4-vinylphenol (DO-1). NaHMDS (2M in THF) (1.17 g, 3.19 mL, 2 molar, 3 Eq, 6.38 mmol) was added to a solution of methyltriphenylphosphonium, Iodide (2.58 g, 3 Eq, 6.38 mmol) in THF (10 mL) and the mixture stirred at rt for 1 h. The reaction was cooled to −40° C. and intermediate (C-2) (500 mg, 1 Eq, 2.13 mmol) in THF (2 mL) added. The mixture was stirred 1 h at -40° C., then allowed to reach rt overnight. HCl 1M (10 mL) was added and the mixture partitioned between EtOAc (25 mL) and water (20 mL). The organic layer was washed with HCl 1M (3×5 mL) and brine. The combined organic layers were collected, dried (MgSO₄), filtered and concentrated in vacuo. The crude product was purified by chromatography on silica gel (40 g cartridge, 0-20% EtOAc/isohexane) to afford the sub-title compound (DO-1) (275 mg, 1.1 mmol, 54%, 97% Purity) as a pale yellow oil. m/z 232.0 (M−H)⁻ (ES−).

Step 2: 2-Nitro-4-(oxiran-2-yl)-6-(trifluoromethyl)phenol (DO-2). To a solution of the product from step 1 above (DO-1) (150 mg, 1 Eq, 643 μmol) in DCM (5 mL) was added mCPBA (317 mg, 70% Wt, 2 Eq, 1.29 mmol). The mixture was stirred at rt over the weekend. The solvent was removed in vacuo to afford the sub-title compound (DO-2) (461 mg, 0.65 mmol, 100%, 35% Purity) as a pale-yellow solid (containing 65% m-Cl benzoic acid), which was used directly in the next step without purification. m/z 248.1 (M−H)⁻ (ES−).

Step 3: 4-(1-(Azetidin-1-yl)-2-hydroxyethyl)-2-nitro-6-(trifluoromethyl)phenol (DO-4). The product from step 2 above (DO-2) (550 mg, 35% Wt, 1 Eq, 773 μmol), azetidine (DO-3) (441 mg, 521 μL, 10 Eq, 7.73 mmol) and EtOH (3 mL) were mixed together and stirred at rt overnight. The solvent was removed in vacuo. The crude product was loaded onto a column of SCX (3.0 g) in MeOH. The column was washed with MeOH and then the product was eluted with 0.7 M ammonia in MeOH. The resultant mixture was concentrated in vacuo. The crude residue was purified by chromatography on silica gel (12 g cartridge, 0-30% MeOH/DCM) to afford the sub-title compound (DO-4) (32 mg, 95 μmol, 12%, 91% Purity) as a pale yellow solid. m/z 307.2 (M+H)⁺ (ES+).

Step 4: 2-Amino-4-(1-(azetidin-1-yl)-2-hydroxyethyl)-6-(trifluoromethyl)phenol (DO-5). To a solution of the product from step 3 above (DO-4) (27 mg, 1 Eq, 88 μmol) in MeOH (1.0 mL) was added Pt/C 28 (3.0 mg, 0.17 Eq, 15 μmol). The mixture was stirred at rt under 4 atm of H₂ (0.18 mg, 1 Eq, 88 μmol) for 2 h. The catalyst was removed by filtration and the solvent was removed in vacuo to afford the sub-title compound (DO-5) (24 mg, 86 μmol, 98%, 99% Purity) as a pale-yellow solid. m/z 277.2 (M+H)⁺ (ES+). ¹H NMR (400 MHz, Methanol-d4) δ 6.89 (d, J=2.1 Hz, 1H), 6.85 (d, J=2.2 Hz, 1H), 3.69 (dd, J=11.2, 6.1 Hz, 1H), 3.59 (dd, J=11.2, 5.9 Hz, 1H), 3.48-3.36 (m, 3H), 3.33-3.25 (m, 2H), 2.14 (p, J=7.3 Hz, 2H). 4 Exchangeable protons not visible.

Step 5: (R)-2-(Azetidin-1-yl)-2-(2-(4′-fluoro-2′-(4-methyl-4H-1,2,4-triazol-3-yl)-[1,1′-biphenyl]-3-yl)-7-(trifluoromethyl)benzo[d]oxazol-5-yl)ethan-1-ol (DO-6) and (S)-2-(Azetidin-1-yl)-2-(2-(4′-fluoro-2′-(4-methyl-4H-1,2,4-triazol-3-yl)-[1,1′-biphenyl]-3-yl)-7-(trifluoromethyl)benzo[d]oxazol-5-yl)ethan-1-ol (DO-7). To a solution of intermediate (B-5) (44 mg, 83% Wt, 1.2 Eq, 0.13 mmol) in MeOH (2 mL) was added the product from step 4 above (DO-5) (30 mg, 1 Eq, 0.11 mmol). The resulting mixture was stirred at 45° C. for 12 h. After concentration in vacuo, the residue was dissolved in DCM (2 mL) and DDQ (27 mg, 1.1 Eq, 0.12 mmol) was then added. After stirring at rt for 1 h, the resulting mixture was diluted with additional EtOAc (25 mL) and washed sequentially with sat. NaHCO₃ (2×5 mL) and brine (5 mL). The organic layer was dried (Na₂SO₄). The crude product was purified by chromatography on RP Flash C18 (12 g cartridge, 30-65% MeCN/10 mM ammonium bicarbonate) to afford a 26 mg of 97% pure racemic product. The residue was dissolved to 26 mg/mL in DCM/MeOH (1:1) with sonication, filtered and was then separated by chiral SFC on a Sepiatec with UV detection by DAD at 220 nm, 40° C., 120 bar. The column was an IC 10×250 mm, 5 um, flow rate 20 mL/min at 50% MeOH (0.03% ammonia), 50% CO₂. The clean fractions were pooled, rinsed with MeOH and concentrated to dryness using a rotary evaporator at 40° C. The residues were re-dissolved in MeOH/DCM (1:1) transferred into final vials and evaporated on a Biotage V10. The samples were then further dried in a vacuum oven at 30° C./5 mbar overnight to afford (R)-2-(azetidin-1-yl)-2-(2-(4′-fluoro-2′-(4-methyl-4H-1,2,4-triazol-3-yl)-[1,1′-biphenyl]-3-yl)-7-(trifluoromethyl)benzo[d]oxazol-5-yl)ethan-1-ol (DO-6) (6.6 mg, 12 μmol, 11%, 99% Purity) and (S)-2-(azetidin-1-yl)-2-(2-(4′-fluoro-2′-(4-methyl-4H-1,2,4-triazol-3-yl)-[1,1′-biphenyl]-3-yl)-7-(trifluoromethyl)benzo[d]oxazol-5-yl)ethan-1-ol (DO-7) (6.4 mg, 12 μmol, 11%, 99% Purity) as very pale yellow solid. Isomer 1: m/z 538.3 (M+H)⁺ (ES+). ¹H NMR (400 MHz, Methanol-d4) δ 8.41 (s, 1H), 8.23 (dt, J=8.0, 1.4 Hz, 1H), 8.09 (t, J=1.7 Hz, 1H), 8.00 (d, J=1.5 Hz, 1H), 7.81 (dd, J=8.7, 5.5 Hz, 1H), 7.74 (s, 1H), 7.63-7.54 (m, 2H), 7.53-7.44 (m, 2H), 3.77 (td, J=8.1, 6.0 Hz, 1H), 3.69-3.58 (m, 2H), 3.37 (d, J=6.9 Hz, 1H), 3.31-3.21 (m, 5H), 2.15 (p, J=7.1 Hz, 2H). Exchangeable proton not visible and one proton under the solvent peaks. Isomer 2: m/z 538.3 (M+H)⁺ (ES+). ¹H NMR (400 MHz, Methanol-d4) δ 8.41 (s, 1H), 8.24 (dt, J=8.0, 1.3 Hz, 1H), 8.09 (t, J=1.7 Hz, 1H), 8.00 (t, J=1.1 Hz, 1H), 7.81 (dd, J=8.6, 5.5 Hz, 1H), 7.74 (s, 1H), 7.64-7.54 (m, 2H), 7.53-7.44 (m, 2H), 3.77 (td, J=8.0, 6.1 Hz, 1H), 3.68-3.58 (m, 2H), 3.37 (d, J=7.1 Hz, 1H), 3.27 (d, J=9.2 Hz, 5H), 2.15 (p, J=7.1 Hz, 2H). Exchangeable proton not visible and one proton under the solvent peaks.

### Example 187: (1R,2R)-2-(((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)amino)-1-methylcyclopentan-1-ol (DP-5) and (1S,2R)-2-(((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)amino)-1-methylcyclopentan-1-ol (DP-6)

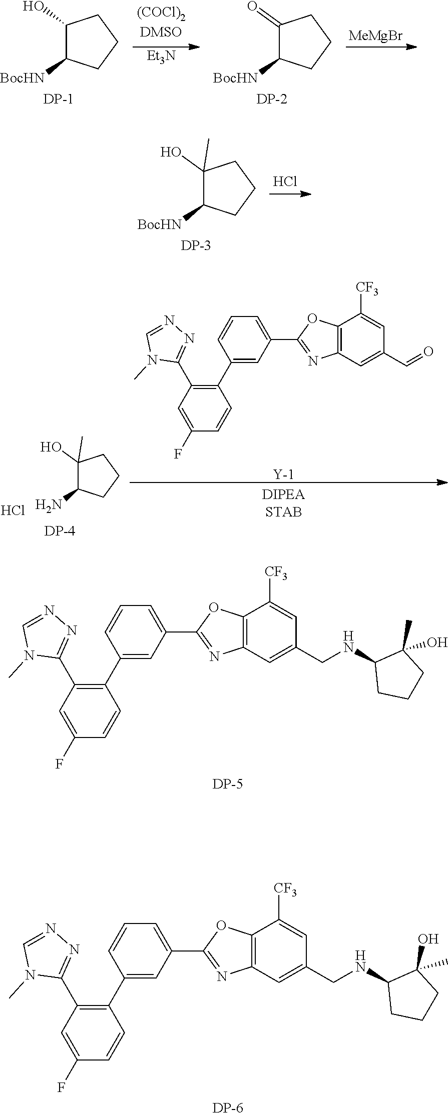

Step 1: tert-Butyl (R)-(2-oxocyclopentyl)carbamate (DP-2). A mixture of oxalyl chloride (473 mg, 326 μL, 1.5 Eq, 3.73 mmol) in THF (8 mL) was cooled to −72° C. under nitrogen and treated dropwise with DMSO (582 mg, 529 μL, 3 Eq, 7.45 mmol). The mixture was stirred for 5 min, whereupon tert-butyl ((1R,2R)-2-hydroxycyclopentyl)carbamate (DP-1) (500 mg, 1 Eq, 2.48 mmol) was added. Stirring was continued at −75° C. for 45 min. Et₃N (1.38 g, 1.90 mL, 5.5 Eq, 13.7 mmol) was added slowly. After the addition was complete, the reaction was allowed to warm slowly to rt overnight. Water (5 mL) was added and the reaction was concentrated in vacuo to remove THF. The mixture was extracted with EtOAc (3×10 mL). The combined organic portions were dried (MgSO₄), filtered and concentrated. The crude product was purified by chromatography (ELSD) on silica gel (24 g cartridge, 10-40% EtOAc/isohexane) to afford the sub-title compound (DP-2) (273 mg, 1.3 mmol, 53%, 97% Purity) as a colourless solid. ¹H NMR (400 MHz, DMSO-d6) δ 6.96 (d, J=8.3 Hz, 1H), 3.76 (dt, J=10.6, 8.2 Hz, 1H), 2.27-2.17 (m, 1H), 2.14-2.00 (m, 2H), 1.95-1.81 (m, 1H), 1.80-1.64 (m, 2H), 1.37 (s, 9H).

Step 2: tert-Butyl ((1R)-2-hydroxy-2-methylcyclopentyl)carbamate (DP-3). To a solution of the product from step 1 above (DP-2) (178 mg, 1 Eq, 893 μmol) in THF (5 mL) at 0° C. under nitrogen was added MeMgBr (3M in Et₂O) (266 mg, 744 μL, 3 molar, 2.5 Eq, 2.23 mmol) dropwise. The reaction was warmed to rt and stirred for 16 h. The reaction was carefully quenched with sat. aq. NH₄Cl (3 mL) and water (3 mL). EtOAc (5 mL) and 1 N HCl (1 mL) were added to dissolve a white precipitate. The layers were separated, and the aqueous portion extracted with EtOAc (2×5 mL). The combined organic portions were dried (MgSO₄), filtered and concentrated. The crude product was purified by chromatography on silica gel (ELSD) (12 g cartridge, 0-60% EtOAc/isohexane) the sub-title compound (DP-3) (121 mg, 0.49 mmol, 55%, 88% Purity) as a yellow oil. Mixture of isomers cis/trans 58:42.

Step 3: (2R)-2-Amino-1-methylcyclopentan-1-ol, HCl (DP-4). HCl (in dioxane) (205 mg, 1.41 mL, 4 molar, 10 Eq, 5.62 mmol) was added to the products from step 2 above (DP-3) (121 mg, 1 Eq, 562 μmol) in dioxane (2 mL) and the mixture stirred at rt for 16 h. The bulk solvent was removed from the reaction mixture to afford crude sub-title compound (DP-4) (82 mg, 0.54 mmol, 95%, 99% Purity) as a brown oil which was used crude without further purification or analysis. Mixture of isomers cis/trans 44:56.

Step 4: (1R,2R)-2-(((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)amino)-1-methylcyclopentan-1-ol (DP-5) and (1S,2R)-2-(((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)amino)-1-methylcyclopentan-1-ol (DP-6). A solution of intermediate (Y-1) (60 mg, 85% Wt, 1 Eq, 0.11 mmol), the product from step 3 above (DP-4) (33 mg, 2 Eq, 0.22 mmol) and DIPEA (42 mg, 57 μL, 3.0 Eq, 0.33 mmol) in CHCl₃ (1 mL) was stirred for 1 h at rt. NaBH(OAc)₃ (70 mg, 3.0 Eq, 0.33 mmol) was added and the reaction mixture was stirred at rt overnight. The reaction mixture was diluted with EtOAc (10 mL) and washed with sat. aq. sol. of NaHCO₃ (10 mL) and brine (10 mL). The organic layer was extracted, dried (MgSO₄), filtered and concentrated in vacuo. The crude product was purified by chromatography on silica gel (12 g cartridge, 0-10% MeOH/DCM) followed by preparative HPLC (Waters, Basic (0.1% ammonia), Basic, Waters X-Bridge Prep-C18, 5 μm, 30×100 mm column, 0-100% MeCN in Water) to afford: (1R,2R)-2-(((2-(4′-fluoro-2′-(4-methyl-4H-1,2,4-triazol-3-yl)-[1,1′-biphenyl]-3-yl)-7-(trifluoromethyl)benzo[d]oxazol-5-yl)methyl)amino)-1-methylcyclopentan-1-ol (DP-5) (12.79 mg, 22 μmol, 20%, 98% Purity). m/z 566.2 (M+H)⁺ (ES+). ¹HNMR (400 MHz, DMSO-d6) δ 8.42 (s, 1H), 8.13 (dt, J=7.9, 1.4 Hz, 1H), 8.10-8.07 (m, 1H), 7.91 (t, J=1.8 Hz, 1H), 7.80-7.75 (m, 2H), 7.64-7.53 (m, 3H), 7.38 (dt, J=7.9, 1.4 Hz, 1H), 4.28 (s, 1H), 4.00-3.86 (m, 2H), 3.20 (s, 3H), 2.71-2.65 (m, 1H), 1.92-1.81 (m, 1H), 1.59-1.46 (m, 4H), 1.36-1.26 (m, 1H), 1.15 (s, 3H). One exchangeable not observed. (1S,2R)-2-(((2-(4′-fluoro-2′-(4-methyl-4H-1,2,4-triazol-3-yl)-[1,1′-biphenyl]-3-yl)-7-(trifluoromethyl)benzo[d]oxazol-5-yl)methyl)amino)-1-methylcyclopentan-1-ol (DP-6) (1.12 mg, 1.9 μmol, 1.8%, 97% Purity). m/z 566.2 (M+H)⁺ (ES+). ¹H NMR (400 MHz, Methanol-d4) δ 8.41 (s, 1H), 8.24 (dt, J=8.0, 1.3 Hz, 1H), 8.09 (t, J=1.8 Hz, 1H), 8.04 (s, 1H), 7.85-7.79 (m, 2H), 7.64-7.53 (m, 2H), 7.53-7.44 (m, 2H), 4.13-3.99 (m, 2H), 3.27 (s, 3H), 2.75-2.69 (m, 1H), 2.10-1.99 (m, 1H), 1.85-1.74 (m, 2H), 1.74-1.48 (m, 3H), 1.33 (s, 3H). Two exchangeable protons not observed.

### Example 188: 1-((((2-(4′-Fluoro-2′-(4-methyl-4H-1,2,4-triazol-3-yl)-[1,1′-biphenyl]-3-yl)benzo[d]oxazol-5-yl)methyl)amino)methyl)cyclobutan-1-ol (DQ-1)

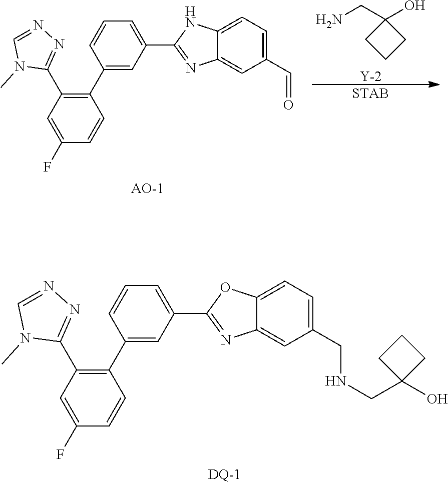

A solution of intermediate (AO-1) (30 mg, 99o Wt, 1 Eq, 75 μmol) and 1-(aminomethyl)cyclobutan-1-ol (Y-2) (15 mg, 2.0 Eq, 0.15 mmol) in CHCl₃ (1 mL) was stirred 1h at rt, then NaBH(OAc)₃ (47 mg, 3.0 Eq, 0.22 mmol) was added. The reaction mixture was stirred at rt overnight. The reaction mixture was diluted with EtOAc (25 mL) and transferred into a separating funnel. The layer was washed with sat. aq. sol. of NaHCO₃ (2×5 mL) and brine. The combined organic layers were collected, dried (MgSO₄), filtered and concentrated in vacuo. The crude product was purified by chromatography on silica gel (12 g cartridge, 0-30% (0.7 M ammonia/MeOH)/DCM) to afford the title compound (DQ-1) (24 mg, 49 μmol, 66%, 99% Purity) as a clear white solid. m/z 484.0 (M+H)⁺ (ES+). ¹H NMR (400 MHz, Methanol-d4) δ 8.40 (s, 1H), 8.21 (dt, J=7.9, 1.4 Hz, 1H), 8.09 (t, J=1.8 Hz, 1H), 7.83-7.76 (m, 2H), 7.66 (d, J=8.4 Hz, 1H), 7.61-7.53 (m, 2H), 7.48 (dt, J=8.5, 1.9 Hz, 2H), 7.41 (ddd, J=7.8, 1.9, 1.1 Hz, 1H), 4.00 (s, 2H), 3.21 (s, 3H), 2.78 (s, 2H), 2.19-1.99 (m, 4H), 1.77 (dtt, J=11.3, 9.6, 3.8 Hz, 1H), 1.54 (dp, J=11.2, 8.9 Hz, 1H). 2 Exchangeable protons not visible.

### Example 189: (R)-1-((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)pyrrolidine-3-carbonitrile (DR-2)

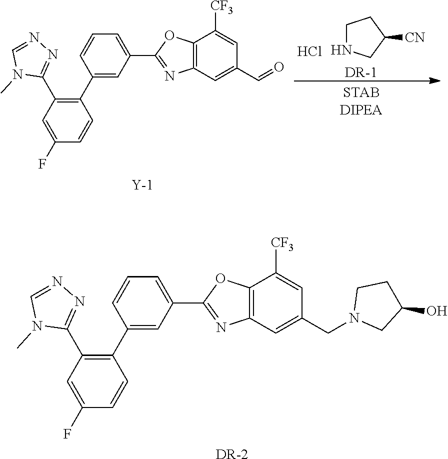

A solution of intermediate (Y-1) (30.5 mg, 90% Wt, 1 Eq, 58.9 μmol), (3R)-3-pyrrolidinecarbonitrile, HCl (DR-1) (15.4 mg, 1.97 Eq, 116 μmol) and DIPEA (37 mg, 50 μL, 4.9 Eq, 0.29 mmol) in CHCl₃ (1.0 mL) was stirred for 1 h at rt. NaBH(OAc)₃ (41.1 mg, 97% Wt, 3.20 Eq, 188 μmol) was added and the reaction mixture was stirred at rt. overnight. The reaction mixture was diluted with EtOAc (5 mL), washed with sat. NaHCO₃ (5 mL) and brine (5 mL), dried (Na₂SO₄), filtered and concentrated in vacuo. The crude product was purified by chromatography on silica gel (4 g cartridge, 0-10% MeOH/DCM) to afford the title compound (DR-2) (5.6 mg, 9.7 μmol, 17%, 95% Purity) as a white solid. m/z 547.3 (M+H)⁺ (ES+). ¹H NMR (400 MHz, DMSO-d6) δ 8.42 (s, 1H), 8.13 (dt, J=8.0, 1.3 Hz, 1H), 8.06 (s, 1H), 7.91 (t, J=1.8 Hz, 1H), 7.77 (dd, J=8.6, 5.7 Hz, 1H), 7.73 (s, 1H), 7.64-7.52 (m, 3H), 7.39 (dt, J=8.0, 1.3 Hz, 1H), 3.84 (s, 2H), 3.20 (s, 3H), 2.81-2.73 (m, 2H), 2.73-2.69 (m, 1H), 2.26-2.15 (m, 1H), 2.01-1.92 (m, 1H). 1×CH₂ peak (2 protons) overlap with DMSO solvent peak.

### Example 190: N-((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)cyclopentanamine (DS-2)

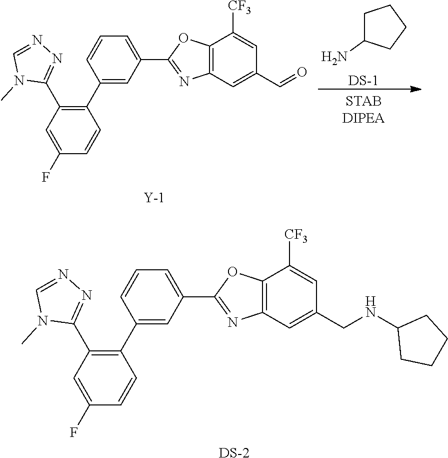

A solution of intermediate (Y-1) (30.8 mg, 90% Wt, 1 Eq, 59.4 μmol), cyclopentylamine (DS-1) (9.5 mg, 11 μL, 1.9 Eq, 0.11 mmol) and DIPEA (22 mg, 29 μL, 2.8 Eq, 0.17 mmol) in CHCl₃ (1.0 mL) was stirred for 1 h at rt. NaBH(OAc)₃ (35.3 mg, 97% Wt, 2.72 Eq, 162 μmol) was added and the reaction mixture was stirred at rt overnight. The reaction mixture was diluted with EtOAc (5 mL), washed with sat. NaHCO₃ (5 mL) and brine (5 mL), dried (Na₂SO₄), filtered and concentrated in vacuo. The crude product was purified by chromatography on silica gel (4 g cartridge, 0-10% MeOH/DCM) to afford the title compound (DS-2) (14.7 mg, 27 μmol, 46%, 99% Purity) as an off-white solid. m/z 536.2 (M+H)⁺ (ES+). ¹H NMR (400 MHz, DMSO-d6) δ 8.42 (s, 1H), 8.13 (dt, J=7.8, 1.4 Hz, 1H), 8.07 (s, 1H), 7.90 (t, J=1.8 Hz, 1H), 7.80-7.74 (m, 2H), 7.65-7.51 (m, 3H), 7.38 (dt, J=7.8, 1.5 Hz, 1H), 3.87 (s, 2H), 3.20 (s, 3H), 3.04-2.97 (m, 1H), 1.76-1.67 (m, 2H), 1.67-1.58 (m, 2H), 1.50-1.41 (m, 2H), 1.41-1.31 (m, 2H). 1× Exchangeable NH proton not observed.

### Example 191: (R)-1-Cyclopropyl-2-(((2-(4′-fluoro-2′-(4-methyl-4H-1,2,4-triazol-3-yl)-[1,1′-biphenyl]-3-yl)-7-(trifluoromethyl)benzo[d]oxazol-5-yl)methyl)amino)ethan-1-ol (DT-2) and (S)-1-Cyclopropyl-2-(((2-(4′-fluoro-2′-(4-methyl-4H-1,2,4-triazol-3-yl)-[1,1′-biphenyl]-3-yl)-7-(trifluoromethyl)benzo[d]oxazol-5-yl)methyl)amino)ethan-1-ol (DT-3)

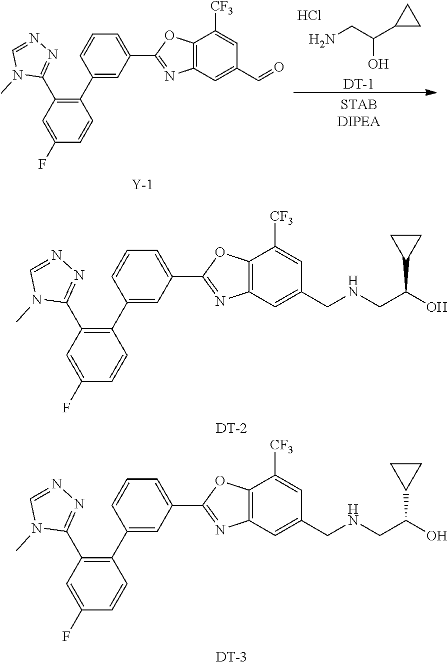

A solution of intermediate (Y-1) (70 mg, 85% Wt, 1 Eq, 0.13 mmol), 2-amino-1-cyclopropylethan-1-ol, HCl (DT-1) (35 mg, 2 Eq, 0.26 mmol) and DIPEA (49 mg, 67 μL, 3.0 Eq, 0.38 mmol) in CHCl₃ (1 mL) was stirred for 1 h at rt. NaBH(OAc)₃ (81 mg, 3.0 Eq, 0.38 mmol) was added and the reaction mixture was stirred at rt overnight. Additional NaBH(OAc)₃ (81 mg, 3.0 Eq, 0.38 mmol) was added and the reaction mixture was stirred at rt for 3 h. The reaction mixture was diluted with EtOAc (10 mL) and washed with sat. aq. sol. of NaHCO₃ (10 mL) and brine (10 mL). The organic layer was extracted, dried (MgSO₄), filtered and concentrated in vacuo. The crude product was purified by chromatography on silica gel (12 g cartridge, 0-100% MeOH/DCM) to afford the title compounds as a racemic mixture. The mixture was dissolved to 20 mg/mL in MeOH, filtered and was then separated by chiral SFC on a Waters prep 15 with UV detection by DAD at 210-400 nm, 40° C., 120 bar. The column was IG 10×250 mm, 5 um, flow rate 15 mL/min at 45% EtOH (0.03% ammonia), 55% CO₂. The clean fractions were pooled, rinsed with MeOH and concentrated to dryness using a rotary evaporator. The residues were re-dissolved in MeOH transferred into final vials and evaporated on a Biotage V10. The samples were then further dried in a vacuum oven at 30° C./5 mbar over night to afford (R)-1-cyclopropyl-2-(((2-(4′-fluoro-2′-(4-methyl-4H-1,2,4-triazol-3-yl)-[1,1′-biphenyl]-3-yl)-7-(trifluoromethyl)benzo[d]oxazol-5-yl)methyl)amino)ethan-1-ol (DT-2) (8.56 mg, 15 μmol, 12%, 98% Purity) and (S)-1-cyclopropyl-2-(((2-(4′-fluoro-2′-(4-methyl-4H-1,2,4-triazol-3-yl)-[1,1′-biphenyl]-3-yl)-7-(trifluoromethyl)benzo[d]oxazol-5-yl)methyl)amino)ethan-1-ol (DT-3) (11.3 mg, 20 μmol, 16%, 98% Purity). Isomer 1: m/z 552.2 (M+H)⁺ (ES+). ¹H NMR (400 MHz, DMSO-d6) δ 8.42 (s, 1H), 8.16-8.10 (m, 1H), 8.09-8.06 (m, 1H), 7.92-7.89 (m, 1H), 7.81-7.75 (m, 2H), 7.64-7.53 (m, 3H), 7.39-7.36 (m, 1H), 4.53 (d, J=4.6 Hz, 1H), 3.91 (s, 2H), 3.20 (s, 3H), 3.05-2.97 (m, 1H), 2.62-2.52 (m, 2H), 0.87-0.77 (m, 1H), 0.37-0.31 (m, 2H), 0.26-0.21 (m, 1H), 0.19-0.13 (m, 1H). One exchangeable proton not observed. Isomer 2: m/z 552.1 (M+H)⁺ (ES+). ¹H NMR (400 MHz, DMSO-d6) δ 8.42 (s, 1H), 8.15-8.11 (m, 1H), 8.08 (s, 1H), 7.91 (t, J=1.9 Hz, 1H), 7.80-7.74 (m, 2H), 7.66-7.52 (m, 3H), 7.41-7.34 (m, 1H), 4.53 (d, J=4.6 Hz, 1H), 3.91 (s, 2H), 3.20 (s, 3H), 3.05-2.97 (m, 1H), 2.63-2.52 (m, 2H), 0.88-0.76 (m, 1H), 0.37-0.30 (m, 2H), 0.27-0.20 (m, 1H), 0.20-0.11 (m, 1H). One exchangeable proton not observed.

### Example 192: (1S,2R)-2-(((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)amino)cyclobutan-1-ol (DU-2)

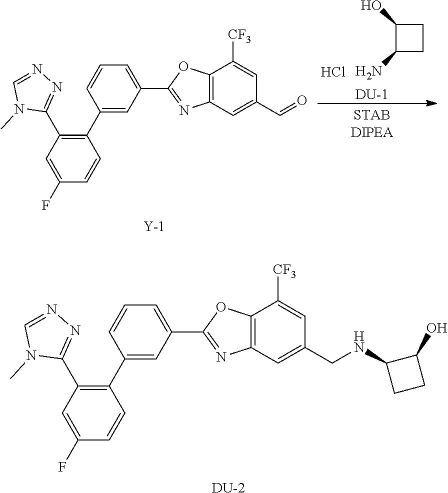

A solution of intermediate (Y-1) (30 mg, 1 Eq, 64 μmol), (1S,2R)-2-aminocyclobutan-1-ol, HCl (DU-1) (16 mg, 2 Eq, 0.13 mmol) and DIPEA (25 mg, 34 μL, 3.0 Eq, 0.19 mmol) in CHCl₃ (1 mL) was stirred for 1 h at rt. NaBH(OAc)₃ (41 mg, 3.0 Eq, 0.19 mmol) was added and the reaction mixture was stirred at rt overnight. The reaction mixture was diluted with EtOAc (10 mL) and washed with sat. aq. sol. of NaHCO₃ (10 mL) and brine (10 mL). The organic layer was extracted, dried (MgSO₄), filtered and concentrated in vacuo. The crude product was purified by chromatography on silica gel (12 g cartridge, 0-30% MeOH/DCM) to afford the title compound (DU-2) (16 mg, 29 μmol, 45%, 97% Purity) as a white solid. m/z 538.2 (M+H)⁺ (ES+). ¹H NMR (400 MHz, DMSO-d6) δ 8.42 (s, 1H), 8.13 (dt, J=7.8, 1.4 Hz, 1H), 8.07 (s, 1H), 7.91 (t, J=1.8 Hz, 1H), 7.80-7.75 (m, 2H), 7.64-7.52 (m, 3H), 7.38 (dt, J=7.9, 1.3 Hz, 1H), 4.90 (d, J=5.8 Hz, 1H), 4.21-4.14 (m, 1H), 3.91 (d, J=13.9 Hz, 1H), 3.81 (d, J=13.8 Hz, 1H), 3.21 (s, 4H), 1.98-1.80 (m, 2H), 1.76-1.66 (m, 2H). 1H under DMSO.

### Example 193: 1-Cyclopentyl-N-((2-(4′-fluoro-2′-(4-methyl-4H-1,2,4-triazol-3-yl)-[1,1′-biphenyl]-3-yl)-7-(trifluoromethyl)benzo[d]oxazol-5-yl)methyl)methanamine (DV-2)

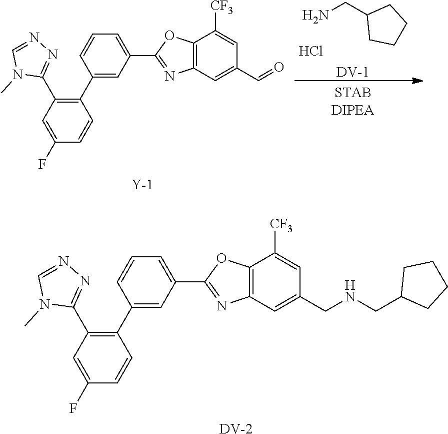

A solution of intermediate (Y-1) (30.2 mg, 90% Wt, 1 Eq, 58.3 μmol), aminomethylcyclopentane, HCl (DV-1) (18.5 mg, 95% Wt, 2.22 Eq, 130 μmol) and DIPEA (37 mg, 50 μL, 4.9 Eq, 0.29 mmol) in CHCl₃ (1.0 mL) was stirred for 1 h at rt. NaBH(OAc)₃ (38.3 mg, 97% Wt, 3.01 Eq, 175 μmol) was added and the reaction mixture was stirred at rt overnight. Additional aminomethylcyclopentane, HCl (DV-1) (17.5 mg, 95% Wt, 2.10 Eq, 123 μmol), DIPEA (22 mg, 30 μL, 3.0 Eq, 0.17 mmol) and NaBH(OAc)₃ (39.0 mg, 97% Wt, 3.06 Eq, 178 μmol) were added. The resulting mixture was stirred at rt for 3 days. The reaction mixture was diluted with EtOAc (5 mL), washed with sat. NaHCO₃ (5 mL) and brine (5 mL), dried (Na₂SO₄), filtered and concentrated in vacuo. The crude product was purified by chromatography on silica gel (4 g cartridge, 0-10% MeOH/DCM) to afford the title compound (DV-2) (14.4 mg, 25 μmol, 44%, 97% Purity) as an off-white solid. m/z 550.3 (M+H)⁺ (ES+). ¹H NMR (400 MHz, DMSO-d6) δ 8.42 (s, 1H), 8.15-8.11 (m, 1H), 8.06 (s, 1H), 7.91 (d, J=1.9 Hz, 1H), 7.80-7.75 (m, 2H), 7.64-7.52 (m, 3H), 7.38 (dd, J=7.8, 1.6 Hz, 1H), 3.88 (s, 2H), 3.20 (s, 3H), 2.40 (d, J=7.1 Hz, 2H), 2.02-1.94 (m, 1H), 1.75-1.65 (m, 2H), 1.58-1.42 (m, 4H), 1.20-1.12 (m, 2H). 1× Exchangeable NH proton not observed.

### Example 194: (2-(4′-Fluoro-2′-(4-methyl-4H-1,2,4-triazol-3-yl)-[1,1′-biphenyl]-3-yl)-7-methoxybenzo[d]oxazol-5-yl)methanol (DW-3)

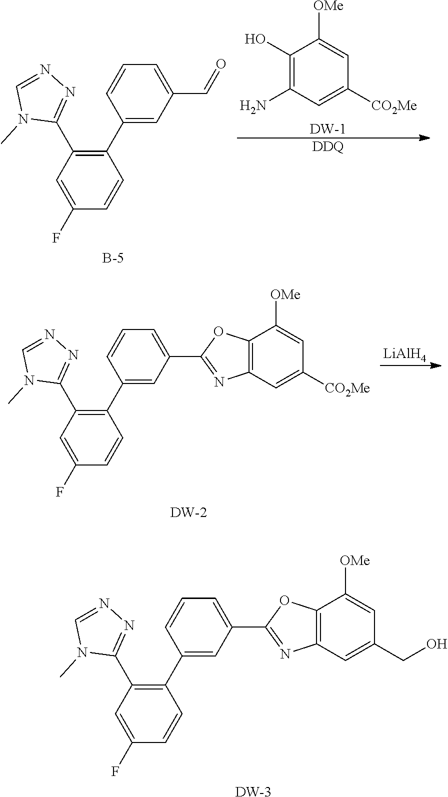

Step 1: Methyl 2-(4′-fluoro-2′-(4-methyl-4H-1,2,4-triazol-3-yl)-[1,1′-biphenyl]-3-yl)-7-methoxybenzo[d]oxazole-5-carboxylate (DW-2). To a solution of intermediate (B-5) (100 mg, 80% Wt, 1 Eq, 284 μmol) in MeOH (5 mL) was added methyl 3-amino-4-hydroxy-5-methoxybenzoate (DW-1) (56.1 mg, 1 Eq, 284 μmol). The resulting mixture was stirred at 45° C. for 12 h. After concentration in vacuo, the residue was dissolved in DCM (5 mL) and DDQ (71.0 mg, 1.1 Eq, 313 μmol) was then added. After stirring at rt for 1 h, the resulting mixture was diluted with additional EtOAc (25 mL) and washed sequentially with saturated NaHCO₃ (3×10 mL) and brine (10 mL). The organic layer was dried (Na₂SO₄). The crude product was purified by chromatography on silica gel (40 g cartridge, 0-30% MeOH/DCM) to afford the sub-title compound (DW-2) (82 mg, 0.16 mmol, 57%, 90% Purity) as a pale brown solid. m/z 459.2 (M+H)⁺ (ES+). ¹H NMR (400 MHz, DMSO-d6) δ 8.42 (s, 1H), 8.14 (dt, J=7.9, 1.4 Hz, 1H), 8.00 (t, J=1.8 Hz, 1H), 7.97 (d, J=1.4 Hz, 1H), 7.78 (dd, J=8.6, 5.6 Hz, 1H), 7.63-7.57 (m, 2H), 7.55-7.52 (m, 2H), 7.30 (dt, J=7.9, 1.3 Hz, 1H), 4.09 (s, 3H), 3.91 (s, 3H), 3.14 (s, 3H).

Step 2: (2-(4′-Fluoro-2′-(4-methyl-4H-1,2,4-triazol-3-yl)-[1,1′-biphenyl]-3-yl)-7-methoxybenzo[d]oxazol-5-yl)methanol (DW-3). To a solution of the product from step 1 above (DW-2) (380 mg, 1 Eq, 829 μmol) in THF (15 mL) was added LiAlH₄ (62.9 mg, 1.66 mL, 1 molar, 2 Eq, 1.66 mmol) dropwise at 0° C. This mixture was stirred 1 h at this temperature. The mixture was quenched with H₂O (0.2 mL), and NaOH (aq. 15%, 0.2 mL), and H₂O (0.6 mL) was added at 0° C. The solvent was removed in vacuo. The crude product was purified by chromatography on silica gel (40 g cartridge, 0-10% MeOH/DCM) to afford the title compound (DW-3) (181 mg, 0.42 mmol, 50%, 99% Purity) as a white solid. m/z 431.1 (M+H)⁺ (ES+). ¹H NMR (400 MHz, DMSO) δ 8.41 (s, 1H), 8.11 (d, J=7.9 Hz, 1H), 7.98 (t, J=1.8 Hz, 1H), 7.78 (dd, J=8.6, 5.6 Hz, 1H), 7.60 (td, J=8.6, 2.9 Hz, 1H), 7.55-7.49 (m, 2H), 7.30 (s, 1H), 7.26 (d, J=7.8 Hz, 1H), 7.04 (s, 1H), 5.33 (t, J=5.8 Hz, 1H), 4.63-4.56 (m, 2H), 4.01 (s, 3H), 3.13 (s, 3H).

### Example 195: (1S,2R)-2-(((2-(4′-Fluoro-2′-(4-methyl-4H-1,2,4-triazol-3-yl)-[1,1′-biphenyl]-3-yl)-7-methoxybenzo[d]oxazol-5-yl)methyl)amino)cyclopentan-1-ol (DX-2)

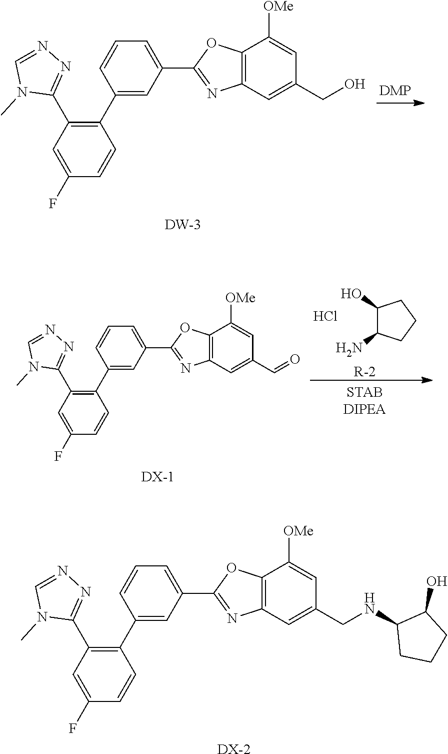

Step 1: 2-(4′-Fluoro-2′-(4-methyl-4H-1,2,4-triazol-3-yl)-[1,1′-biphenyl]-3-yl)-7-methoxybenzo[d]oxazole-5-carbaldehyde (DX-1). Compound (DW-3) (55 mg, 90% Wt, 1 Eq, 0.11 mmol) and DMP (98 mg, 2.0 Eq, 0.23 mmol) were stirred in CHCl₃ (5 mL) at rt overnight. The reaction mixture was diluted with EtOAc (25 mL) and washed with sat. aq. NaHCO₃ (5×5 mL) and brine (10 mL). Organics were separated, dried (MgSO₄), filtered and evaporated to afford the sub-title compound (DX-1) (49 mg, 0.11 mmol, 99%) which was used for next step without further purification.

Step 2: (1S,2R)-2-(((2-(4′-Fluoro-2′-(4-methyl-4H-1,2,4-triazol-3-yl)-[1,1′-biphenyl]-3-yl)-7-methoxybenzo[d]oxazol-5-yl)methyl)amino)cyclopentan-1-ol (DX-2). A solution of the product from step 1 above (DX-1) (30 mg, 1 Eq, 70 μmol), (1S,2R)-2-aminocyclopentan-1-ol, HCl (R-2) (19 mg, 2 Eq, 0.14 mmol) and DIPEA (27 mg, 37 μL, 3.0 Eq, 0.21 mmol) in CHCl₃ (1 mL) was stirred for 1 h at rt. NaBH(OAc)₃ (45 mg, 3.0 Eq, 0.21 mmol) was added and the reaction mixture was stirred at rt overnight. The reaction mixture was diluted with EtOAc (10 mL) and washed with sat. aq. sol. of NaHCO₃ (10 mL) and brine (10 mL). The organic layer was extracted, dried (MgSO₄), filtered and concentrated in vacuo. The crude product was purified by chromatography on silica gel (12 g cartridge, 0-40% MeOH/DCM) to afford the title compound (DX-2) (10.2 mg, 19.9 μmol, 28%, 95% Purity) as a white solid. m/z 514.2 (M+H)⁺ (ES+). ¹H NMR (400 MHz, DMSO-d6) δ 8.41 (d, J=2.0 Hz, 1H), 8.13-8.06 (m, 1H), 7.96 (q, J=2.0 Hz, 1H), 7.77 (ddd, J=8.2, 5.8, 2.1 Hz, 1H), 7.64-7.55 (m, 1H), 7.52 (ddt, J=7.9, 6.2, 2.7 Hz, 2H), 7.39 (s, 1H), 7.30-7.24 (m, 1H), 7.15 (s, 1H), 4.05-3.99 (m, 4H), 3.97-3.87 (m, 2H), 3.16-3.08 (m, 3H), 2.94-2.83 (m, 1H), 1.80-1.57 (m, 4H), 1.53-1.37 (m, 2H). 2H under water peak.

### Example 196: 4-((((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)amino)methyl)tetrahydro-2H-pyran-4-carbonitrile (DY-1)

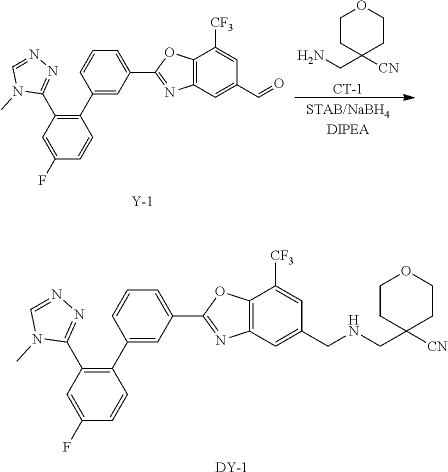

A solution of intermediate (Y-1) (30.1 mg, 90% Wt, 1 Eq, 58.1 μmol), 4-(aminomethyl)oxane-4-carbonitrile (CT-1) (18.7 mg, 95% Wt, 2.18 Eq, 127 μmol) and DIPEA (22 mg, 30 μL, 3.0 Eq, 0.17 mmol) in CHCl₃ (1 mL) was stirred for 1 h at rt. NaBH(OAc)₃ (38.6 mg, 97% Wt, 3.04 Eq, 177 μmol) was added and the reaction mixture was stirred at rt overnight. Additional 4-(aminomethyl)oxane-4-carbonitrile (CT-1) (10.3 mg, 95% Wt, 1.20 Eq, 69.8 μmol) and NaBH(OAc)₃ (39.4 mg, 97% Wt, 3.10 Eq, 180 μmol) were added. The resulting mixture was stirred at rt for 3 days. The reaction was heated at 40° C. for 1 h. The reaction was cooled to rt. NaBH₄ (3.4 mg, 98% Wt, 1.5 Eq, 88 μmol) was added and the resulting mixture was stirred at rt for 24 h. The reaction mixture was diluted with EtOAc (5 mL), washed with sat. NaHCO₃ (5 mL) and brine (5 mL), dried (Na₂SO₄), filtered and concentrated in vacuo. The crude product was purified by chromatography on silica gel (4 g cartridge, 0-10% MeOH/DCM) to afford the title compound (DY-1) (7.3 mg, 12 μmol, 21%, 99% Purity) as an off-white solid. m/z 591.2 (M+H)⁺ (ES+). ¹H NMR (400 MHz, DMSO-d6) δ 8.42 (s, 1H), 8.16-8.09 (m, 2H), 7.91 (t, J=1.8 Hz, 1H), 7.83 (s, 1H), 7.78 (dd, J=8.6, 5.6 Hz, 1H), 7.65-7.52 (m, 3H), 7.38 (dt, J=8.1, 1.3 Hz, 1H), 4.00-3.95 (m, 2H), 3.90-3.82 (m, 2H), 3.51-3.42 (m, 2H), 3.20 (s, 3H), 2.90-2.82 (m, 1H), 2.71-2.64 (m, 2H), 1.88-1.80 (m, 2H), 1.55 (td, J=13.0, 4.6 Hz, 2H)

### Example 197: N-((2-(4′-Fluoro-2′-(4-methyl-4H-1,2,4-triazol-3-yl)-[1,1′-biphenyl]-3-yl)benzo[d]oxazol-5-yl)methyl)-2-methoxyethan-1-amine (DZ-1)

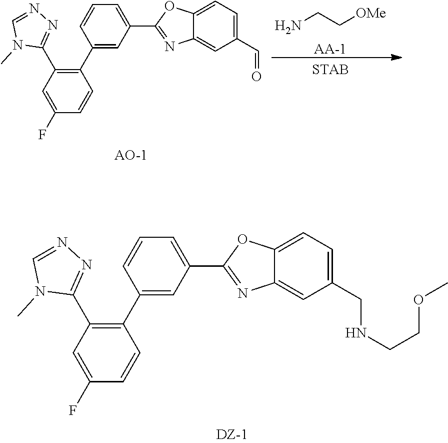

A solution of intermediate (AO-1) (30 mg, 90% Wt, 1 Eq, 68 μmol) and 2-methoxyethan-1-amine (AA-1) (10 mg, 12 μL, 2 Eq, 0.14 mmol) in CHCl₃ (1 mL) was stirred 1 h at rt, then NaBH(OAc)₃ (43 mg, 3.0 Eq, 0.20 mmol) was added. The reaction mixture was stirred at rt overnight. The reaction mixture was diluted with EtOAc (10 mL), washed with sat. aq. sol. of NaHCO₃ (2×5 mL) and brine (5 mL). The organic layer was dried (MgSO₄), filtered and concentrated in vacuo. The crude product was purified by chromatography on silica gel (12 g cartridge, 0-50% MeOH/DCM) to afford the title compound (DZ-1) (14.14 mg, 30 μmol, 44%, 97% Purity) as a white solid. m/z 458.2 (M+H)⁺ (ES+). ¹H NMR (400 MHz, DMSO-d6) δ 8.42 (s, 1H), 8.12 (dt, J=7.9, 1.3 Hz, 1H), 7.97 (t, J=1.8 Hz, 1H), 7.80-7.76 (m, 1H), 7.75-7.69 (m, 2H), 7.60 (td, J=8.6, 2.8 Hz, 1H), 7.57-7.50 (m, 2H), 7.40 (dd, J=8.4, 1.6 Hz, 1H), 7.29-7.25 (m, 1H), 3.84 (s, 2H), 3.41 (t, J=5.7 Hz, 2H), 3.24 (s, 3H) 3.13 (s, 3H), 2.65 (t, J=5.7 Hz, 3H).

### Example 198: (1S,2R)-2-(((S)-1-(2-(4′-Fluoro-2′-(4-methyl-4H-1,2,4-triazol-3-yl)-[1,1′-biphenyl]-3-yl)-7-(trifluoromethyl)benzo[d]oxazol-5-yl)ethyl)amino)cyclopentan-1-ol (EA-1) and (1S,2R)-2-(((R)-1-(2-(4′-Fluoro-2′-(4-methyl-4H-1,2,4-triazol-3-yl)-[1,1′-biphenyl]-3-yl)-7-(trifluoromethyl)benzo[d]oxazol-5-yl)ethyl)amino)cyclopentan-1-ol (EA-2)

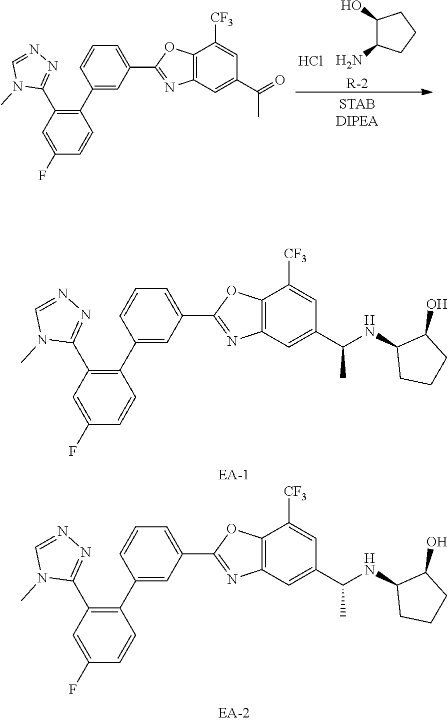

A solution of compound (DK-1) (40 mg, 1 Eq, 83 μmol), (1S,2R)-2-aminocyclopentan-1-ol, HCl (R-2) (17 mg, 1.5 Eq, 0.12 mmol) and DIPEA (32 mg, 44 μL, 3.0 Eq, 0.25 mmol) in CHCl₃ (2 mL) was stirred 1 h at rt, then NaBH(OAc)₃ (53 mg, 3.0 Eq, 0.25 mmol) was added. The reaction mixture was stirred at rt overnight. The reaction mixture was warmed up at 65° C., (1S,2R)-2-aminocyclopentan-1-ol, HCl (R-2) (17 mg, 1.5 Eq, 0.12 mmol), NaBH(OAc)₃ (53 mg, 3.0 Eq, 0.25 mmol), acetic acid (50 mg, 48 μL, 10 Eq, 0.83 mmol) and molecular sieves (4A) were added in the mixture. The reaction mixture was stirred at this temperature for 14 days (additional CHCl₃ was added from time to time to avoid the mixture drying out). The reaction mixture was diluted with EtOAc (25 mL) and transferred into a separating funnel. The layer was washed with sat. aq. sol. of NaHCO₃ (3×5 mL) and brine (10 mL). The combined organic layers were collected, dried (MgSO₄), filtered and concentrated in vacuo. The crude product was loaded onto a column of SCX (500 mg) in MeOH. The column was washed with MeOH and then the product was eluted with 0.7 M ammonia in MeOH. The resultant mixture was concentrated in vacuo to afford a 50% pure product. The crude product was purified by preparative HPLC (Gilson, Basic (0.3% ammonia), Basic, Phenomenex Gemini C18 prep column, 130A, 5 μm, 30 mm×150 mm column, 35-100% MeCN in Water) to afford title compound (1S,2R)-2-(((R)-1-(2-(4′-fluoro-2′-(4-methyl-4H-1,2,4-triazol-3-yl)-[1,1′-biphenyl]-3-yl)-7-(trifluoromethyl)benzo[d]oxazol-5-yl)ethyl)amino)cyclopentan-1-ol (EA-2) (1.4 mg, 2.4 μmol, 2.9%, 98% Purity). m/z 566.2 (M+H)⁺ (ES+). ¹H NMR (400 MHz, DMSO-d6) δ 8.41 (s, 1H), 8.15-8.09 (m, 2H), 7.89 (d, J=2.2 Hz, 1H), 7.82 (s, 1H), 7.77 (dd, J=8.6, 5.6 Hz, 1H), 7.64-7.53 (m, 3H), 7.39 (d, J=8.0 Hz, 1H), 4.40 (s, 1H), 4.05 (d, J=6.6 Hz, 1H), 3.96 (s, 1H), 3.20 (s, 3H), 1.64-1.45 (m, 4H), 1.37-1.26 (m, 5H). 2H under water. The second fraction was further purified by preparative HPLC (Waters, Acidic (0.1% formic acid), Acidic, X-Select CSH C18 ODB prep column, 130A, 5 μm, 30 mm×100 mm column, 10-100% MeCN in Water) to afford the title compound (EA-1) (1S,2R)-2-(((S)-1-(2-(4′-fluoro-2′-(4-methyl-4H-1,2,4-triazol-3-yl)-[1,1′-biphenyl]-3-yl)-7-(trifluoromethyl)benzo[d]oxazol-5-yl)ethyl)amino)cyclopentan-1-ol (5.5 mg, 9.6 μmol, 12%, 99% Purity). m/z 566.3 (M+H)⁺ (ES+). ¹H NMR (400 MHz, DMSO-d6) δ 8.41 (s, 1H), 8.22 (s, 1H), 8.13 (d, J=3.9 Hz, 1H), 7.91-7.87 (m, 1H), 7.81 (s, 1H), 7.79-7.74 (m, 1H), 7.64-7.53 (m, 3H), 7.39 (d, J=7.8 Hz, 1H), 4.11-4.06 (m, 1H), 3.75-3.71 (m, 2H), 3.23-3.19 (m, 4H), 1.75-1.44 (m, 6H), 1.36 (d, J=6.5 Hz, 3H). 1H under water.

### Example 199: (2-(4′-Fluoro-2′-(4-methyl-4H-1,2,4-triazol-3-yl)-[1,1′-biphenyl]-3-yl)-7-(trifluoromethyl)benzo[d]oxazol-5-yl)methanamine (EB-1)

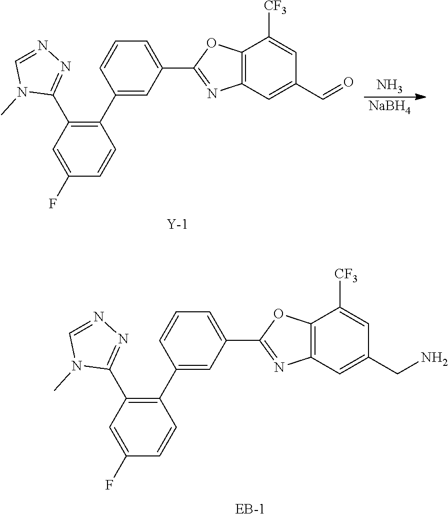

A solution of intermediate (Y-1) (50 mg, 85% Wt, 1 Eq, 91 μmol) and NH₃/MeOH (0.6 g, 5 mL, 7 molar, 4e+2 Eq, 0.04 mol) stirred for 16 h. NaBH₄ (10 mg, 3 Eq, 0.27 mmol) was added to the reaction mixture and stirred for 1 h. The reaction mixture concentrated in vacuo and the crude product was purified by chromatography on silica gel (4 g cartridge, 0-10% (0.7 M NH₃/MeOH)/DCM) and the product-containing fractions were combined and concentrated. The crude was dissolved in MeOH (5 mL) and loaded onto SCX (ca. 0.5 g) and washed with MeOH (15 mL), the product was then eluted with 0.7 M NH₃/MeOH (15 mL) and concentrated in vacuo. The crude product was purified by preparative HPLC (Waters, Basic (0.1% ammonia), Basic, Waters X-Bridge Prep-C18, 5 μm, 30×100 mm column, 0-100% MeCN in water) to afford the title compound (EB-1) (4.73 mg, 10 μmol, 11%, 99% Purity) as a white solid. m/z 468.0 (M+H)⁺ (ES+). ¹H NMR (400 MHz, DMSO) δ 8.42 (s, 1H), 8.13 (dt, J=8.0, 1.4 Hz, 1H), 8.07 (s, 1H), 7.91 (t, J=1.8 Hz, 1H), 7.79 (s, 1H), 7.79-7.75 (m, 1H), 7.63-7.53 (m, 3H), 7.37 (dt, J=7.9, 1.5 Hz, 1H), 3.90 (s, 2H), 3.21 (s, 3H). Two exchangeable protons not observed.

### Example 200: N-((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)methanesulfonamide (EC-1)

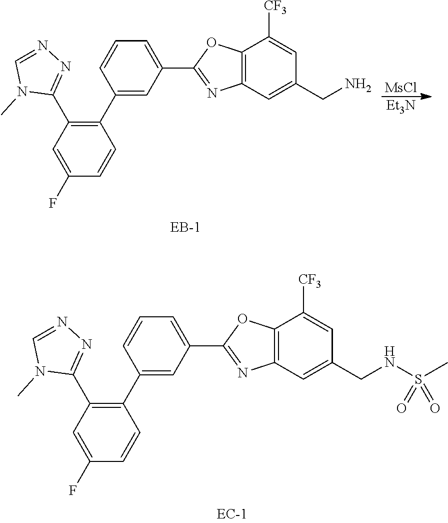

To a solution of compound (EB-1) (30 mg, 85% Wt, 1 Eq, 55 μmol) and Et₃N (6.6 mg, 9.1 μL, 1.2 Eq, 65 μmol) in DCM (2 mL) was added methanesulfonyl chloride (6.9 mg, 1.1 Eq, 60 μmol) and stirred at rt overnight. Water (1 mL) was added, and the organic layer was extracted, dried (phase separator) and concentrated in vacuo. The crude product was purified by preparative HPLC (Waters, Basic (0.1% ammonia), Basic, Waters X-Bridge Prep-C18, 5 μm, 19×50 mm column, 0-100% MeCN in water) to afford the title compound (EC-1) (7.01 mg, 12 μmol, 22%, 95% Purity) as a white solid. m/z 546.0 (M+H)⁺ (ES+). ¹H NMR (400 MHz, DMSO-d6) δ 8.42 (s, 1H), 8.14 (d, J=8.0 Hz, 1H), 8.09 (s, 1H), 7.92 (s, 1H), 7.80-7.75 (m, 2H), 7.64-7.53 (m, 3H), 7.38 (d, J=8.0 Hz, 1H), 4.39 (s, 2H), 3.21 (s, 3H), 2.94 (s, 3H). One exchangeable proton not observed.

### Example 201: (1S,2R)-2-(((7-Fluoro-2-(4′-fluoro-2′-(4-methyl-4H-1,2,4-triazol-3-yl)-[1,1′-biphenyl]-3-yl)benzo[d]oxazol-5-yl)methyl)amino)cyclopentan-1-ol (ED-7)

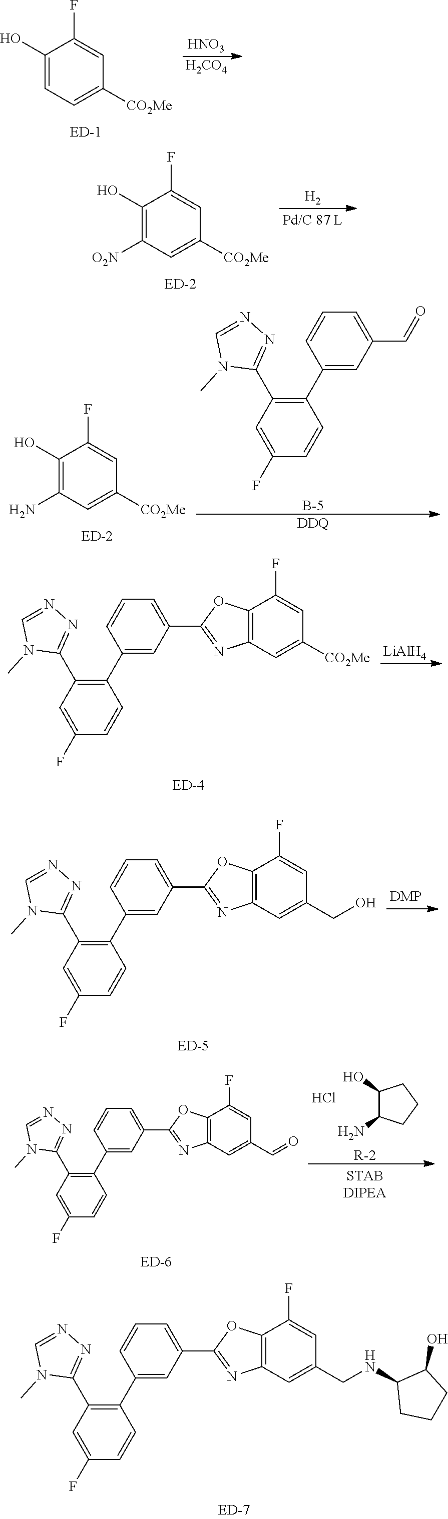

Step 1: Methyl 3-fluoro-4-hydroxy-5-nitrobenzoate (ED-2). To a mixture of methyl 3-fluoro-4-hydroxybenzoate (ED-1) (500 mg, 1 Eq, 2.94 mmol) suspended in conc. H₂SO₄ (10.1 g, 5.51 mL, 35 Eq, 103 mmol) was added HNO₃ (463 mg, 307 μL, 2.5 Eq, 7.35 mmol) slowly at 0° C. The resulting suspension was stirred at this temperature for 5 min. The mixture was poured into ice water (10 mL). A precipitation was observed, the product was filtered and dried to afford the sub-title compound (ED-2) (254 mg, 1.2 mmol, 40%, 99% Purity) as a yellow solid. m/z 216.1 (M+H)⁺ (ES+). ¹H NMR (400 MHz, DMSO) δ 8.25 (t, J=1.9 Hz, 1H), 8.01 (dd, J=10.9, 2.1 Hz, 1H), 3.86 (s, 3H). Exchangeable H not observed.

Step 2: Methyl 3-amino-5-fluoro-4-hydroxybenzoate (ED-3). To a solution of the product from step 1 above (ED-2) (254 mg, 1 Eq, 1.18 mmol) in MeOH (5 mL) was added Pd/C 87 L (30 mg, 0.24 Eq, 0.28 mmol). The reaction mixture was stirred at rt with 5 atm of H₂ for 16 h. The catalyst was filtered and the solvent was removed in vacuo to afford the sub-title compound (ED-3) (218 mg, 1.0 mmol, 88%, 88% Purity) as a brown solid. m/z 186.1 (M+H)⁺ (ES+). ¹H NMR (400 MHz, DMSO) δ 7.11 (dd, J=2.0, 1.1 Hz, 1H), 6.92 (dd, J=11.0, 2.1 Hz, 1H), 3.76 (s, 3H). Exchangeable 3H under DMSO.

Step 3: Methyl 7-fluoro-2-(4′-fluoro-2′-(4-methyl-4H-1,2,4-triazol-3-yl)-[1,1′-biphenyl]-3-yl)benzo[d]oxazole-5-carboxylate (ED-4). To a solution of intermediate (B-5) (197.3 mg, 80% Wt, 1 Eq, 561.1 μmol) in MeOH (5.0 mL) was added the product from step 2 above (ED-3) (150 mg, 1.44 Eq, 810 μmol). The resulting mixture was stirred at 45° C. for 3 d. The reaction mixture was cooled to rt and the volatiles were removed in vacuo. The residue was re-dissolved in DCM (5.0 mL). DDQ (150.2 mg, 98% Wt, 1.156 Eq, 648.4 μmol) was added and the resulting mixture was stirred at rt for 5 h. Additional DDQ (145.1 mg, 98% Wt, 1.116 Eq, 626.4 μmol) was added and the resulting mixture was stirred at rt overnight. The reaction mixture was diluted with DCM (10 mL), washed with sat. NaHCO₃ (3×10 mL) and brine. The organic layers were collected, dried (Na₂SO₄), filtered and concentrated in vacuo. The crude product was purified by chromatography on silica gel (24 g cartridge, 0-10% MeOH/DCM) to afford the sub-title compound (ED-4) (55.1 mg, 96 μmol, 17%, 78% Purity) as a dark brown solid. m/z 447.1 (M+H)⁺ (ES+). ¹H NMR (400 MHz, DMSO-d6) δ 8.42 (s, 1H), 8.22 (d, J=1.3 Hz, 1H), 8.17 (dt, J=7.8, 1.3 Hz, 1H), 8.03 (t, J=1.8 Hz, 1H), 7.91 (dd, J=10.6, 1.4 Hz, 1H), 7.79 (dd, J=8.6, 5.6 Hz, 1H), 7.63-7.51 (m, 3H), 7.34 (dt, J=7.9, 1.3 Hz, 1H), 3.92 (s, 3H), 3.16 (s, 3H).

Step 4: (7-fluoro-2-(4′-fluoro-2′-(4-methyl-4H-1,2,4-triazol-3-yl)-[1,1′-biphenyl]-3-yl)benzo[d]oxazol-5-yl)methanol (ED-5). To a solution of the product from step 3 above (55.1 mg, 78% Wt, 1 Eq, 96.3 μmol) in THF (2.0 mL) was added LiAlH₄ in THF (3.65 mg, 96.3 μL, 1 molar, 1 Eq, 96.3 μmol) dropwise at 0° C. This mixture was stirred at 0° C. for 1 h. The mixture was quenched with H₂O (0.5 mL) and NaOH (2 M, 0.5 mL) at 0° C. MgSO₄ was added. The mixture was warmed up to rt, stirred for 15 min, filtered and the solvent was removed in vacuo. The crude product was purified by chromatography on silica gel (12 g cartridge, 0-10% MeOH/DCM) to afford the sub-title compound (ED-5) (53.8 mg, 90 μmol, 93%, 70% Purity) as a pale tan solid. m/z 419.1 (M+H)⁺ (ES+). ¹H NMR (400 MHz, DMSO-d6) δ 8.42 (s, 1H), 8.14 (dt, J=7.9, 1.4 Hz, 1H), 8.00 (t, J=1.8 Hz, 1H), 7.79 (dd, J=8.6, 5.7 Hz, 1H), 7.63-7.50 (m, 4H), 7.34-7.27 (m, 2H), 5.45 (t, J=5.8 Hz, 1H), 4.62 (d, J=5.7 Hz, 2H), 3.15 (s, 3H).

Step 5: 7-Fluoro-2-(4′-fluoro-2′-(4-methyl-4H-1,2,4-triazol-3-yl)-[1,1′-biphenyl]-3-yl)benzo[d]oxazole-5-carbaldehyde (ED-6). To a stirred solution of the product from step 4 above (ED-5) (53.8 mg, 70% Wt, 1 Eq, 90.0 μmol) in dry DCM (3.0 mL) at rt was added Dess-Martin periodinane (96.1 mg, 2.52 Eq, 227 μmol). The reaction mixture was stirred at rt for 1 h. The reaction mixture was diluted with DCM (5 mL), washed with sat. NaHCO₃ (5 mL) and brine (5 mL). The organic layer was dried (Na₂SO₄), filtered and concentrated in vacuo. The crude product was purified by chromatography on silica gel (12 g cartridge, 0-10% MeOH/DCM) to afford the sub-title compound (19.4 mg, 33 μmol, 36%, 70% Purity) as a pale-yellow solid. m/z 417.0 (M+H)⁺ (ES+). ¹H NMR (400 MHz, DMSO-d6) δ 10.10 (d, J=2.0 Hz, 1H), 8.43 (s, 1H), 8.29 (d, J=1.2 Hz, 1H), 8.20-8.17 (m, 1H), 8.04 (s, 1H), 7.91 (dd, J=10.2, 1.2 Hz, 1H), 7.79 (dd, J=8.7, 5.8 Hz, 1H), 7.63-7.49 (m, 3H), 7.37-7.32 (m, 1H), 3.16 (s, 3H).

Step 6: (1S,2R)-2-(((7-Fluoro-2-(4′-fluoro-2′-(4-methyl-4H-1,2,4-triazol-3-yl)-[1,1′-biphenyl]-3-yl)benzo[d]oxazol-5-yl)methyl)amino)cyclopentan-1-ol (ED-7). A solution of the product from step 5 above (ED-6) (19.4 mg, 70% Wt, 1 Eq, 32.6 μmol), (1S,2R)-2-aminocyclopentan-1-ol, HCl (R-2) (13.4 mg, 2.99 Eq, 97.4 μmol) and DIPEA (22 mg, 30 μL, 5.3 Eq, 0.17 mmol) in CHCl₃ (3.0 mL) was stirred 1 h at rt. NaBH(OAc)₃ (33.2 mg, 97% Wt, 4.66 Eq, 152 μmol) was added and the reaction mixture was stirred at rt overnight. The reaction mixture was diluted with EtOAc (2 mL), washed with sat. NaHCO₃ (2 mL) and brine (2 mL), dried (Na₂SO₄), filtered and concentrated in vacuo. The crude product was purified by chromatography on silica gel (4 g cartridge, 0-20% MeOH/DCM) to afford the title compound (ED-7) (7.7 mg, 15 μmol, 46%, 98% Purity) as an off-white solid. m/z 502.2 (M+H)⁺ (ES+). ¹H NMR (400 MHz, Methanol-d4) δ 8.38 (s, 1H), 8.24-8.19 (m, 1H), 8.11-8.08 (m, 1H), 7.78 (dd, J=8.6, 5.4 Hz, 1H), 7.60-7.51 (m, 3H), 7.46 (dd, J=8.8, 2.8 Hz, 1H), 7.43-7.39 (m, 1H), 7.31 (d, J=11.0 Hz, 1H), 4.17-4.13 (m, 1H), 3.98 (d, J=13.2 Hz, 1H), 3.90 (d, J=13.3 Hz, 1H), 3.20 (s, 3H), 2.99-2.94 (m, 1H), 1.95-1.88 (m, 1H), 1.86-1.71 (m, 3H), 1.60-1.48 (m, 2H). 2× exchangeable protons not observed.

### Example 202: N-((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)-3-methoxypropan-1-amine (EE-2)

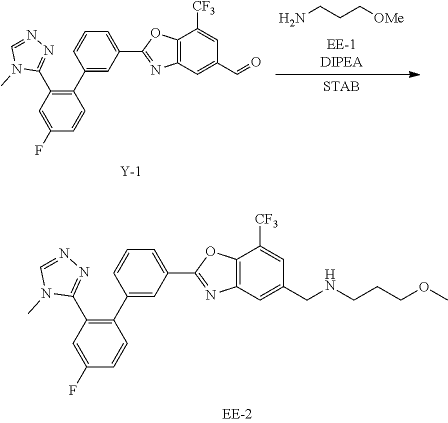

A solution of intermediate (Y-1) (31.1 mg, 90% Wt, 1 Eq, 60.0 μmol), 3-methoxy-propylamine (EE-1) (11 mg, 13 μL, 99% Wt, 2.1 Eq, 0.13 mmol) and DIPEA (22 mg, 30 μL, 2.9 Eq, 0.17 mmol) in CHCl₃ (1.0 mL) was stirred for 1 h at rt. NaBH(OAc)₃ (39.1 mg, 97% Wt, 2.98 Eq, 179 μmol) was added and the reaction mixture was stirred at rt overnight. Additional 3-methoxy-propylamine (EE-1) (11 mg, 13 μL, 99% Wt, 2.1 Eq, 0.13 mmol) and NaBH(OAc)₃ (42.0 mg, 97% Wt, 3.20 Eq, 192 μmol) was added and the resulting mixture was stirred at rt for 3 days. The reaction mixture was diluted with EtOAc (5 mL), washed with sat. NaHCO₃ (5 mL) and brine (5 mL), dried (Na₂SO₄), filtered and concentrated in vacuo. The crude product was purified by chromatography on silica gel (4 g cartridge, 0-10% MeOH/DCM) to afford the title compound (EE-2) (15.5 mg, 28 μmol, 47%, 98% Purity) as an off-white solid. m/z 540.2 (M+H)⁺ (ES+). ¹H NMR (400 MHz, DMSO-d6) δ 8.42 (s, 1H), 8.15-8.11 (m, 1H), 8.06 (s, 1H), 7.91 (t, J=1.8 Hz, 1H), 7.80-7.75 (m, 2H), 7.64-7.53 (m, 3H), 7.40-7.36 (m, 1H), 3.88 (s, 2H), 3.37 (t, J=6.4 Hz, 2H), 3.22-3.18 (m, 6H), 2.56-2.52 (m, 2H), 1.70-1.63 (m, 2H). 1× exchangeable NH proton not observed.

### Example 203: 1-(((1-(2-(4′-Fluoro-2′-(4-methyl-4H-1,2,4-triazol-3-yl)-[1,1′-biphenyl]-3-yl)-7-(trifluoromethyl)benzo[d]oxazol-5-yl)ethyl)amino)methyl)cyclobutan-1-ol, formate (EF-1)

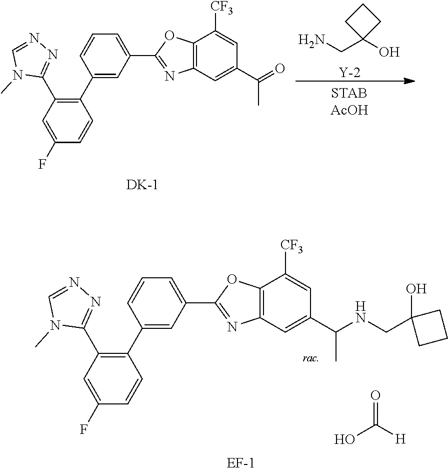

A solution of compound (DK-1) (40 mg, 1 Eq, 83 μmol) and 1-(aminomethyl)cyclobutan-1-ol (Y-2) (13 mg, 1.5 Eq, 0.12 mmol) in CHCl₃ (1 mL) was stirred 1 h at rt, then NaBH(OAc)₃ (53 mg, 3.0 Eq, 0.25 mmol) was added. The reaction mixture was stirred at rt overnight. The reaction mixture was warmed to 65° C., additional 1-(aminomethyl)cyclobutan-1-ol (Y-2) (13 mg, 1.5 Eq, 0.12 mmol), AcOH (10 mg, 9.5 μL, 2.0 Eq, 0.17 mmol), NaBH(OAc)₃ (53 mg, 3.0 Eq, 0.25 mmol) and molecular sieves (4A) were added. The reaction mixture was stirred at this temperature for 14 days (CHCl₃ was added from time to time to avoid the mixture drying out). The reaction mixture was diluted with EtOAc (25 mL) and transferred into a separating funnel. The layer was washed with sat. aq. sol. of NaHCO₃ (3×5 mL) and brine (10 mL). The combined organic layers were collected, dried (MgSO₄), filtered and concentrated in vacuo. The crude product was loaded onto a column of SCX (500 mg) in MeOH. The column was washed with MeOH and then the product was eluted with 0.7 M NH₃ in MeOH. The resultant mixture was concentrated in vacuo to afford a 16% pure product. The crude product was purified by preparative HPLC (Waters, acidic (0.1% formic acid), acidic, Waters X-Select CSH C18 ODB prep column, 130A, 5 μm, 30 mm×100 mm column, 10-100% MeCN in water) to afford the title compound (EF-1) (2.5 mg, 4.0 μmol, 5.2%, 98% Purity) as a white solid. m/z 566.2 (M+H)⁺ (ES+). ¹H NMR (400 MHz, DMSO-d6) δ 8.42-8.40 (m, 1H), 8.13 (dt, J=7.9, 1.4 Hz, 1H), 8.10 (s, 1H), 7.90 (t, J=1.8 Hz, 1H), 7.80 (s, 1H), 7.77 (dd, J=8.6, 5.7 Hz, 1H), 7.64-7.53 (m, 3H), 7.38 (dt, J=7.9, 1.5 Hz, 1H), 3.97 (q, J=6.5 Hz, 1H), 3.20 (s, 3H), 2.48-2.45 (m, 1H), 2.30 (d, J=11.6 Hz, 1H), 2.05-1.96 (m, 1H), 1.88 (td, J=9.6, 6.0 Hz, 3H), 1.63-1.52 (m, 1H), 1.34 (d, J=6.6 Hz, 3H), 1.29-1.20 (m, 1H). 2H under water.

### Example 204: (S)-2-(2-(4′-Fluoro-2′-(4-methyl-4H-1,2,4-triazol-3-yl)-[1,1′-biphenyl]-3-yl)-7-(trifluoromethyl)benzo[d]oxazol-5-yl)-2-methoxyethan-1-ol (EG-2) and (R)-2-(2-(4′-fluoro-2′-(4-methyl-4H-1,2,4-triazol-3-yl)-[1,1′-biphenyl]-3-yl)-7-(trifluoromethyl)benzo[d]oxazol-5-yl)-2-methoxyethan-1-ol (EG-3)

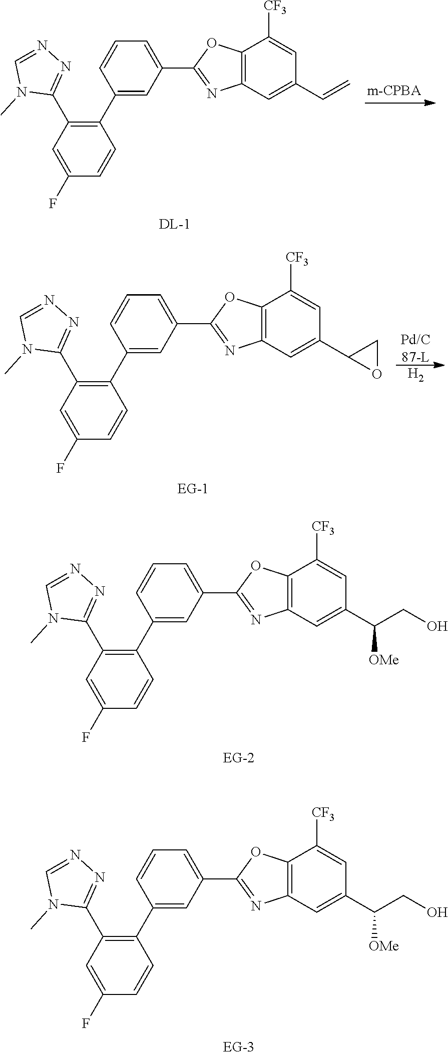

Step 1: 2-(4′-Fluoro-2′-(4-methyl-4H-1,2,4-triazol-3-yl)-[1,1′-biphenyl]-3-yl)-5-(oxiran-2-yl)-7-(trifluoromethyl)benzo[d]oxazole (EG-1). To a solution of compound (DL-1) (40 mg, 99% Wt, 1 Eq, 85 μmol) in DCM (5 mL) was added mCPBA (42 mg, 70% Wt, 2 Eq, 0.17 mmol). The mixture was stirred at rt overnight. Additional mCPBA (42 mg, 70% Wt, 2 Eq, 0.17 mmol) was added in the reaction mixture. The reaction mixture was stirred at rt for 2 h. The solvent was removed in vacuo to afford the sub-title compound (EG-1) (130 mg, 81 μmol, 95%, 30% Purity) in mixture with 3-chlorobenzoic acid as a pale yellow solid. m/z 481.6 (M+H)⁺ (ES+).

Step 2: (S)-2-(2-(4′-Fluoro-2′-(4-methyl-4H-1,2,4-triazol-3-yl)-[1,1′-biphenyl]-3-yl)-7-(trifluoromethyl)benzo[d]oxazol-5-yl)-2-methoxyethan-1-ol (EG-2) and (R)-2-(2-(4′-fluoro-2′-(4-methyl-4H-1,2,4-triazol-3-yl)-[1,1′-biphenyl]-3-yl)-7-(trifluoromethyl)benzo[d]oxazol-5-yl)-2-methoxyethan-1-ol (EG-3). To a solution of the product from step 1 above (EG-1) (130 mg, 30% Wt, 1 Eq, 81.2 μmol) in MeOH (2 mL) was added Pd/C 87 L (8 mg, 0.9 Eq, 0.08 mmol). The reaction mixture was stirred at 45° C. with 5 atm of H₂ for 16 h. The catalyst was filtered and the solvent was removed in vacuo. The crude product was purified by chromatography on silica gel (12 g cartridge, 0-30% MeOH/DCM) to afford racemic product with a purity of 84%. The crude product was purified by chiral SFC on a Waters prep 15 with UV detection by DAD at 210-400 nm, 40° C., 120 bar. The column was an IG 10×250 mm, 5 μM, flow rate 15 mL/min at 35% MeOH (0.03% ammonia), 65% CO₂ affording the title compounds (S)-2-(2-(4′-fluoro-2′-(4-methyl-4H-1,2,4-triazol-3-yl)-[1,1′-biphenyl]-3-yl)-7-(trifluoromethyl)benzo[d]oxazol-5-yl)-2-methoxyethan-1-ol (EG-2) (2.4 mg, 4.5 μmol, 5.7%, 97% Purity) and (R)-2-(2-(4′-fluoro-2′-(4-methyl-4H-1,2,4-triazol-3-yl)-[1,1′-biphenyl]-3-yl)-7-(trifluoromethyl)benzo[d]oxazol-5-yl)-2-methoxyethan-1-ol (EG-3) (2.65 mg, 5.0 μmol, 6.3%, 97% Purity) as white solids. Isomer 1: m/z 513.2 (M+H)⁺ (ES+). ¹H NMR (400 MHz, DMSO-d6) δ 8.42 (s, 1H), 8.13 (dt, J=7.9, 1.4 Hz, 1H), 8.04 (s, 1H), 7.90 (t, J=1.8 Hz, 1H), 7.77 (dd, J=8.6, 5.6 Hz, 1H), 7.70 (s, 1H), 7.64-7.53 (m, 3H), 7.40 (dt, J=7.8, 1.4 Hz, 1H), 4.89 (t, J=5.7 Hz, 1H), 4.46 (t, J=5.5 Hz, 1H), 3.63 (dt, J=11.3, 5.6 Hz, 1H), 3.54 (dt, J=11.3, 5.4 Hz, 1H), 3.24 (s, 3H), 3.21 (s, 3H). Isomer 2: m/z 513.2 (M+H)⁺ (ES+). ¹H NMR (400 MHz, DMSO-d6) δ 8.42 (s, 1H), 8.13 (dt, J=7.9, 1.3 Hz, 1H), 8.04 (d, J=1.4 Hz, 1H), 7.90 (q, J=2.8 Hz, 1H), 7.77 (dd, J=8.7, 5.7 Hz, 1H), 7.70 (s, 1H), 7.63-7.53 (m, 3H), 7.40 (dt, J=8.0, 1.3 Hz, 1H), 4.93-4.85 (m, 1H), 4.46 (t, J=5.5 Hz, 1H), 3.67-3.59 (m, 1H), 3.58-3.48 (m, 1H), 3.24 (s, 3H), 3.21 (s, 3H).

### Example 205: 2-(4′-Fluoro-2′-(4-methyl-4H-1,2,4-triazol-3-yl)-[1,1′-biphenyl]-3-yl)-5-(((3aR,6aS)-tetrahydro-2H-cyclopenta[d]oxazol-3(3aH)-yl)methyl)-7-(trifluoromethyl)benzo[d]oxazole (EH-1)

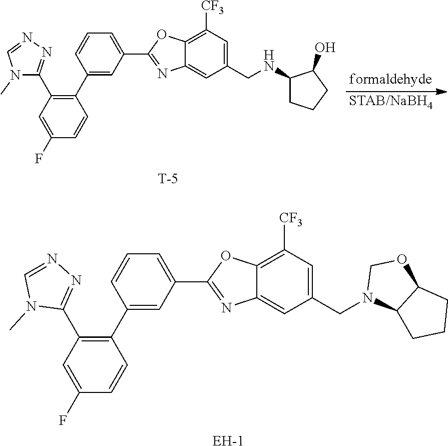

A solution of compound (T-5) (20.8 mg, 1 Eq, 37.7 μmol), formaldehyde solution in H₂O (6.12 mg, 5.62 μL, 37% Wt, 2 Eq, 75.4 μmol) and DIPEA (15 mg, 20 μL, 3.0 Eq, 0.11 mmol) in CHCl₃ (3.0 mL) was stirred 1 h at rt. NaBH(OAc)₃ (29.4 mg, 97% Wt, 3.57 Eq, 135 μmol) was added and the reaction mixture was stirred at rt overnight. Additional formaldehyde solution in H₂O (3.06 mg, 2.81 μL, 37% Wt, 1 Eq, 37.7 μmol), NaBH(OAc)₃ (37.2 mg, 97% Wt, 4.51 Eq, 170 μmol) and DIPEA (15 mg, 20 μL, 3.0 Eq, 0.11 mmol) were added and the resulting mixture was stirred at rt for 3 h. NaBH₄ (1.46 mg, 1.36 μL, 98% Wt, 1 Eq, 37.7 μmol) was added and the resulting mixture was stirred at rt overnight. The reaction mixture was diluted with EtOAc (2 mL), washed with sat. NaHCO₃ (2 mL) and brine (2 mL), dried (Na₂SO₄), filtered and concentrated in vacuo. The crude product was purified by chromatography on silica gel (12 g cartridge, 0-10% MeOH/DCM) to afford the title compound (EH-1) (13.8 mg, 24 μmol, 64%) as an off-white solid. m/z 564.3 (M+H)⁺ (ES+). ¹H NMR (400 MHz, Methanol-d4) δ 8.38 (s, 1H), 8.21 (dt, J=7.9, 1.4 Hz, 1H), 8.06 (t, J=1.7 Hz, 1H), 8.01 (s, 1H), 7.81-7.74 (m, 2H), 7.61-7.51 (m, 2H), 7.49-7.41 (m, 2H), 4.60-4.55 (m, 1H), 4.38 (d, J=5.2 Hz, 1H), 4.22 (d, J=5.2 Hz, 1H), 3.96 (d, J=13.6 Hz, 1H), 3.89 (d, J=13.6 Hz, 1H), 3.50-3.43 (m, 1H), 3.25 (s, 3H), 1.81-1.61 (m, 4H), 1.59-1.51 (m, 1H), 1.51-1.44 (m, 1H).

### Example 206: 2-(((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)amino)-N,N-dimethylacetamide (EI-4)

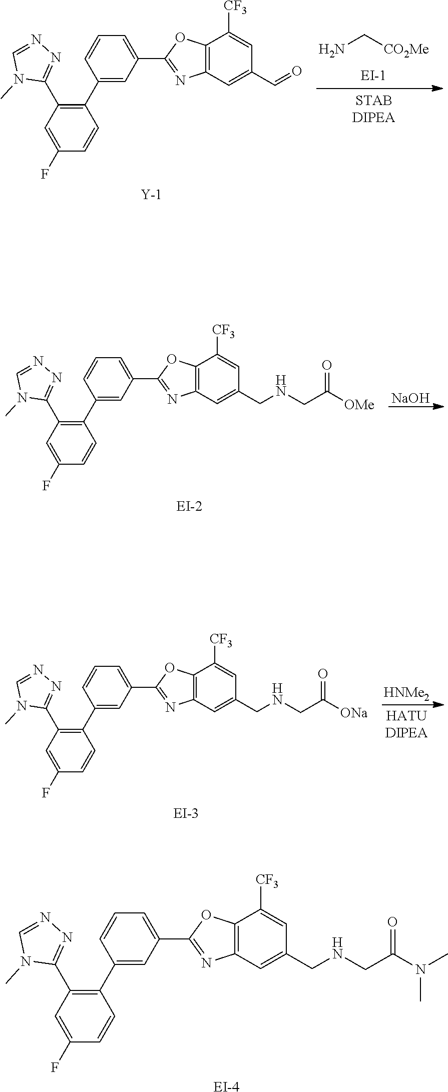

Step 1: Methyl ((2-(4′-fluoro-2′-(4-methyl-4H-1,2,4-triazol-3-yl)-[1,1′-biphenyl]-3-yl)-7-(trifluoromethyl)benzo[d]oxazol-5-yl)methyl)glycinate (EI-2). A solution of intermediate (Y-1) (61.7 mg, 90% Wt, 1 Eq, 119 μmol), DIPEA (46.2 mg, 62.2 μL, 3.0 Eq, 357 μmol) and methyl glycinate, HCl (EI-1) (29.9 mg, 2 Eq, 238 μmol) in CHCl₃ (3 mL) was stirred for 1 h at rt. NaBH(OAc)₃ (75.7 mg, 3.0 Eq, 357 μmol) was added and the reaction mixture was stirred at rt overnight. The reaction mixture was diluted with EtOAc (5 mL) and washed with a sat. aq sol. of NaHCO₃ (5 mL) and brine (5 mL). The organic layer was extracted, dried (MgSO₄), and concentrated in vacuo. The crude product was purified by chromatography on silica gel (12 g cartridge, 0-10% MeOH/DCM) to afford the sub-title compound (E1-2) (50.8 mg, 80 μmol, 67%, 85% Purity) as a pale yellow oil. m/z 540.1 (M+H)⁺ (ES+). ¹H NMR (400 MHz, MeOD) δ 8.41 (s, 1H), 8.25-8.21 (m, 1H), 8.09 (t, J=1.8 Hz, 1H), 8.01 (d, J=1.3 Hz, 1H), 7.81 (dd, J=8.7, 5.4 Hz, 1H), 7.77 (s, 1H), 7.62-7.54 (m, 2H), 7.52-7.44 (m, 2H), 4.00 (s, 2H), 3.76 (s, 3H), 3.47 (s, 2H), 3.27 (s, 3H). One exchangeable proton not observed.

Step 2: ((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)glycine, 2Na (EI-3). To a solution of the product from step 1 above (EI-2) (34 mg, 1 Eq, 63 μmol) in THF (0.5 mL)/MeOH (0.5 mL)/Water (0.25 mL) was added NaOH (5.0 mg, 63 μL, 2 molar, 2.0 Eq, 0.13 mmol). This mixture was stirred at rt for 3 h. The reaction mixture was concentrated in vacuo to afford the sub-title compound (EI-3) (33 mg, 58 μmol, 92%, 100% Purity) as a white solid. The material was used directly without any purification. m/z 525.8 (M+H)⁺ (ES+).

Step 3: 2-(((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)amino)-N,N-dimethylacetamide (EI-4). To a solution of the product from step 2 above (EI-3) (36 mg, 1 Eq, 69 μmol) in DCM (1 mL) were added HATU (39 mg, 1.5 Eq, 0.10 mmol) and DIPEA (44 mg, 60 μL, 5 Eq, 0.34 mmol). This mixture was stirred 20 min at rt then dimethylamine (6.2 mg, 69 μL, 2 molar, 2 Eq, 0.14 mmol) was added. The mixture was stirred at rt for 16 h. The reaction mixture was diluted with EtOAc (5 mL) and washed with sat. aq. sol. of NH₄Cl (2×5 mL). The organic layer was extracted, dried (MgSO₄), filtered and concentrated in vacuo. The crude product was purified by chromatography on silica gel (4 g cartridge, 0-10% MeOH/DCM) to afford the title compound (EI-4) (15.09 mg, 26 μmol, 38%, 95% Purity) as a white solid. m/z 553.2 (M+H)⁺ (ES+). ¹H NMR (400 MHz, DMSO) δ 8.42 (s, 1H), 8.13 (dt, J=7.8, 1.4 Hz, 1H), 8.09 (s, 1H), 7.91 (t, J=1.8 Hz, 1H), 7.82-7.74 (m, 2H), 7.64-7.53 (m, 3H), 7.39 (dt, J=7.8, 1.5 Hz, 1H), 3.98 (s, 2H), 3.48 (s, 2H), 3.21 (s, 3H), 2.89 (s, 3H), 2.84 (s, 3H). One exchangeable proton not observed.

### Example 207: (S)—N-((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)-1-methoxypropan-2-amine (EJ-2)

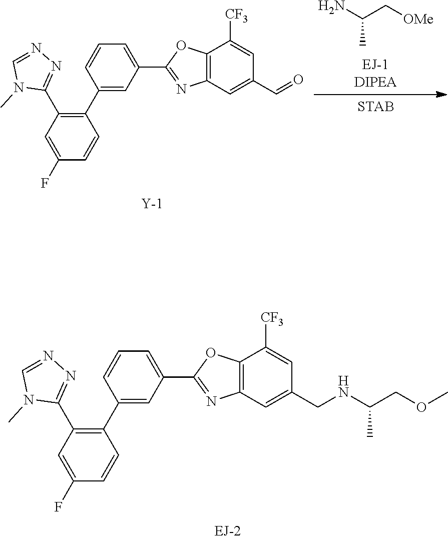

A solution of intermediate (Y-1) (30 mg, 90% Wt, 1 Eq, 58 μmol), DIPEA (22 mg, 30 μL, 3.0 Eq, 0.17 mmol) and (S)-1-methoxypropan-2-amine (EJ-1) (10 mg, 2 Eq, 0.12 mmol) in CHCl3 (3 mL) was stirred for 1 h at rt. NaBH(OAc)₃ (37 mg, 3.0 Eq, 0.17 mmol) was added and the reaction mixture was stirred at rt overnight. The reaction mixture was diluted with EtOAc (5 mL) and washed with a sat. aq sol. of NaHCO₃ (5 mL) and brine (5 mL). The organic layer was extracted, dried (MgSO₄), and concentrated in vacuo. The crude product was purified by chromatography on silica gel (12 g cartridge, 0-10% MeOH/DCM) to afford the title compound (EJ-2) (17.02 mg, 30 μmol, 52%, 95% Purity) as a white solid. m/z 540.3 (M+H)⁺ (ES+). ¹H NMR (400 MHz, DMSO) δ 8.42 (s, 1H), 8.13 (dt, J=7.8, 1.4 Hz, 1H), 8.07 (s, 1H), 7.91 (t, J=1.8 Hz, 1H), 7.80-7.74 (m, 2H), 7.64-7.51 (m, 3H), 7.37 (dt, J=7.8, 1.4 Hz, 1H), 3.99-3.87 (m, 2H), 3.29-3.25 (m, 1H), 3.23 (s, 3H), 3.20 (s, 3H), 3.19-3.16 (m, 1H), 2.76 (q, J=6.2 Hz, 1H), 0.99 (d, J=6.3 Hz, 3H). One exchangeable proton not observed.

### Example 208: (1S,2R)-2-(((2-(4′-Fluoro-2′-(4-methyl-4H-1,2,4-triazol-3-yl)-[1,1′-biphenyl]-3-yl)benzo[d]thiazol-6-yl)methyl)amino)cyclopentan-1-ol (EK-5)

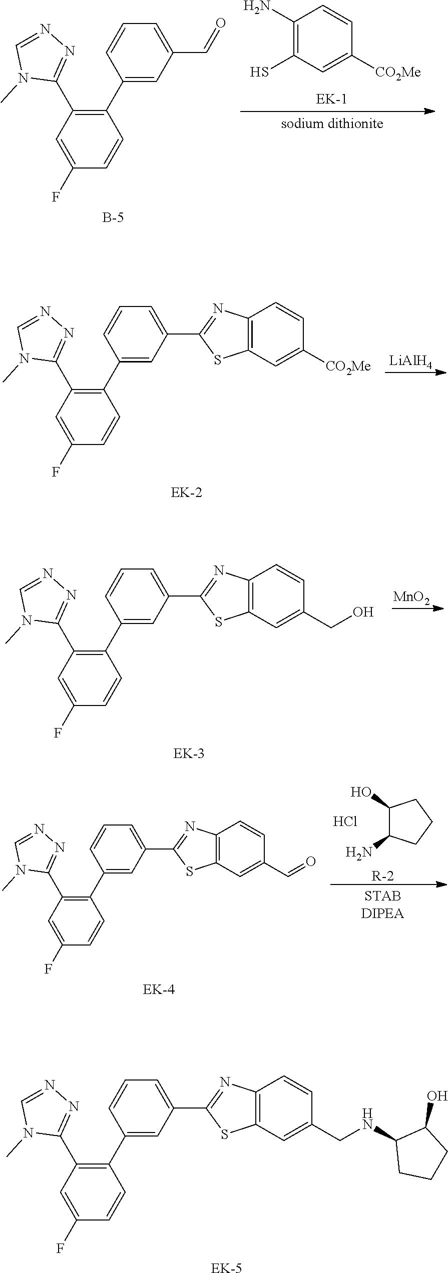

Step 1: Methyl 2-(4′-fluoro-2′-(4-methyl-4H-1,2,4-triazol-3-yl)-[1,1′-biphenyl]-3-yl)benzo[d]thiazole-6-carboxylate (EK-2). Sodium dithionite (190 mg, 76.0 μL, 2.0 Eq, 1.09 mmol) in Water (1 mL) was added to a solution of methyl 4-amino-3-mercaptobenzoate (EK-1) (100 mg, 1 Eq, 546 μmol) and intermediate (B-5) (192 mg, 80% Wt, 1 Eq, 546 μmol) in EtOH (1 mL) and stirred at 80° C. for 16 h. The reaction was allowed to cool to rt and filtered. The collected solid was washed with water and then repeatedly evaporated from EtOH to remove residual water to afford the sub-title compound (EK-2) (105 mg, 0.23 mmol, 42%, 98% Purity) as a pale pink solid. m/z 445.2 (M+H)⁺ (ES+). ¹H NMR (400 MHz, DMSO) δ 8.86-8.81 (m, 1H), 8.44 (s, 1H), 8.19-8.13 (m, 1H), 8.13-8.04 (m, 2H), 7.85-7.75 (m, 2H), 7.60 (td, J=8.6, 2.8 Hz, 1H), 7.56-7.50 (m, 2H), 7.34 (dt, J=7.9, 1.3 Hz, 1H), 3.91 (s, 3H), 3.17 (s, 3H).

Step 2: (2-(4′-Fluoro-2′-(4-methyl-4H-1,2,4-triazol-3-yl)-[1,1′-biphenyl]-3-yl)benzo[d]thiazol-6-yl)methanol (EK-3). A solution of LiAlH₄ in THF (8.88 mg, 234 μL, 1 molar, 1.0 Eq, 234 μmol) was added to a stirred solution of the product from step 1 above (EK-2) (105 mg, 99% Wt, 1 Eq, 234 μmol) in THF (4 mL) at 0° C. The reaction mixture was stirred at 0° C. for 90 min. The reaction mixture was diluted with THF (5 ml) before being vigorously stirred and sodium sulfate decahydrate (c. 300 mg) added portion-wise at 0° C. The reaction mixture was partitioned between EtOAc (15 mL) and water (15 mL). The biphasic mixture was separated, and the aqueous layer was re-extracted with EtOAc (15 mL). The organic extracts were combined, dried (MgSO₄) and concentrated in vacuo to afford the sub-title compound (EK-3) (52 mg, 0.11 mmol, 49%, 92% Purity) as a flocculent pale-yellow solid. m/z 417.2 (M+H)⁺ (ES+).

Step 3: 2-(4′-Fluoro-2′-(4-methyl-4H-1,2,4-triazol-3-yl)-[1,1′-biphenyl]-3-yl)benzo[d]thiazole-6-carbaldehyde (EK-4). The product from step 2 above (EK-3) (54 mg, 0.12 mmol, 100%, 90% Purity) and MnO₂ (0.30 g, 60 μL, 30 Eq, 3.4 mmol) in CHCl₃ (5 mL) was stirred at 40° C. for 4 h. The reaction mixture was then left to stir at rt over the weekend. The reaction mixture was filtered through a small Celite plug (chased with 10 mL DCM). The filtrate was evaporated to dryness to afford the sub-title compound (EK-4) (54 mg, 0.12 mmol, 100%, 90% Purity) as a pale-green foam. m/z 415.2 (M+H)⁺ (ES+). ¹H NMR (400 MHz, DMSO) δ 10.12 (s, 1H), 8.79 (d, J=1.6 Hz, 1H), 8.44 (s, 1H), 8.23 (d, J=8.4 Hz, 1H), 8.14-8.02 (m, 2H), 7.87-7.76 (m, 2H), 7.62 (dd, J=8.6, 2.8 Hz, 1H), 7.59-7.51 (m, 2H), 7.36 (dt, J=7.9, 1.3 Hz, 1H), 3.17 (s, 3H).

Step 4: (1S,2R)-2-(((2-(4′-Fluoro-2′-(4-methyl-4H-1,2,4-triazol-3-yl)-[1,1′-biphenyl]-3-yl)benzo[d]thiazol-6-yl)methyl)amino)cyclopentan-1-ol (EK-5). A mixture of the product from step 3 above (EK-4) (27 mg, 90% Wt, 1 Eq, 59 μmol), (1S,2R)-2-aminocyclopentan-1-ol, HCl (R-2) (8.1 mg, 1 Eq, 59 μmol) and DIPEA (23 mg, 31 μL, 3.0 Eq, 0.18 mmol) in CHCl₃ (2 mL) was stirred at rt for 5 min. NaBH(OAc)₃ (75 mg, 6.0 Eq, 0.35 mmol) was then added and the resultant suspension stirred at rt overnight. Additional (1S,2R)-2-aminocyclopentan-1-ol, HCl (R-2) (8.1 mg, 1 Eq, 59 μmol), DIPEA (23 mg, 31 μL, 3.0 Eq, 0.18 mmol) and NaBH(OAc)₃ (75 mg, 6.0 Eq, 0.35 mmol) were added to the reaction mixture and stirring continued at 40° C. overnight. NaBH₄ (11 mg, 5.0 Eq, 0.29 mmol) was added and stirring continued at rt for 3 h. The reaction mixture then was diluted with DCM (4 mL) and washed with sat. aq. NaHCO₃ (10 mL). The organic phase was separated and evaporated to afford the crude product. The crude product was purified by preparative HPLC (Waters, Basic (0.1% ammonia), Basic, Waters X-Bridge BEH C18 ODB Prep-C18, 5 μm, 30×100 mm column, 25-100% MeCN in water) to afford the title compound (EK-5) (13 mg, 25 μmol, 43%, 100% Purity) as a colourless foam. m/z 500.2 (M+H)⁺ (ES+). ¹H NMR (400 MHz, DMSO) δ 8.43 (s, 1H), 8.09 (d, J=1.6 Hz, 1H), 8.06-7.95 (m, 2H), 7.82-7.73 (m, 2H), 7.60 (td, J=8.6, 2.9 Hz, 1H), 7.57-7.47 (m, 3H), 7.30 (dt, J=8.0, 1.3 Hz, 1H), 4.38 (d, J=3.4 Hz, 1H), 3.99-3.94 (m, 1H), 3.93-3.79 (m, 2H), 3.15 (s, 3H), 2.82-2.78 (m, 1H), 2.19-2.15 (m, 1H), 1.77-1.51 (m, 4H), 1.48-1.36 (m, 2H).

### Example 209: N-((2-(4′-Fluoro-2′-(4-methyl-4H-1,2,4-triazol-3-yl)-[1,1′-biphenyl]-3-yl)-7-methoxybenzo[d]oxazol-5-yl)methyl)-2-methoxyethan-1-amine (EL-1)

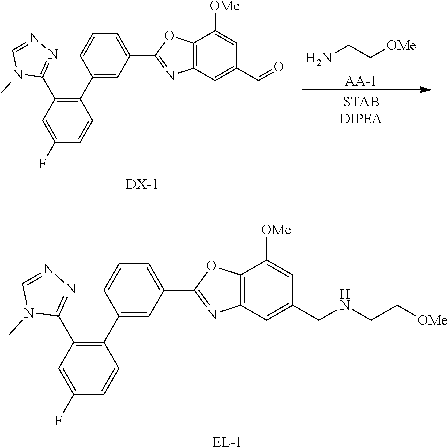

A solution of intermediate (DX-1) (20 mg, 1 Eq, 47 μmol), 2-methoxyethan-1-amine (AA-1) (7.0 mg, 8.1 μL, 2 Eq, 93 μmol) and DIPEA (18 mg, 24 μL, 3.0 Eq, 0.14 mmol) in CHCl₃ (1 mL) was stirred for 1 h at rt. NaBH(OAc)₃ (30 mg, 3.0 Eq, 0.14 mmol) was added and the reaction mixture was stirred at rt overnight. The reaction mixture was diluted with EtOAc (10 mL) and washed with sat. aq. sol. of NaHCO₃ (10 mL) and brine (10 mL). The organic layer was extracted, dried (MgSO₄), filtered and concentrated in vacuo. The crude product was purified by chromatography on silica gel (12 g cartridge, 0-30% MeOH/DCM) to afford the title compound (EL-1) (10 mg, 19 μmol, 42%, 95% Purity) as a yellow solid. m/z 488.2 (M+H)⁺ (ES+). ¹H NMR (400 MHz, DMSO) δ 8.41 (s, 1H), 8.10 (dt, J=7.9, 1.4 Hz, 1H), 7.96 (t, J=1.8 Hz, 1H), 7.78 (dd, J=8.6, 5.6 Hz, 1H), 7.59 (td, J=8.5, 2.7 Hz, 1H), 7.55-7.49 (m, 2H), 7.33 (d, J=1.3 Hz, 1H), 7.27 (dt, J=7.8, 1.4 Hz, 1H), 7.09 (d, J=1.3 Hz, 1H), 4.01 (s, 3H), 3.85 (s, 2H), 3.43 (t, J=5.7 Hz, 2H), 3.25 (s, 3H), 3.13 (s, 3H), 2.69 (t, J=5.7 Hz, 2H). NH not observed.

### Example 210: 1-Cyclobutyl-N-((2-(4′-fluoro-2′-(4-methyl-4H-1,2,4-triazol-3-yl)-[1,1′-biphenyl]-3-yl)-7-(trifluoromethyl)benzo[d]oxazol-5-yl)methyl)methanamine (EM-2)

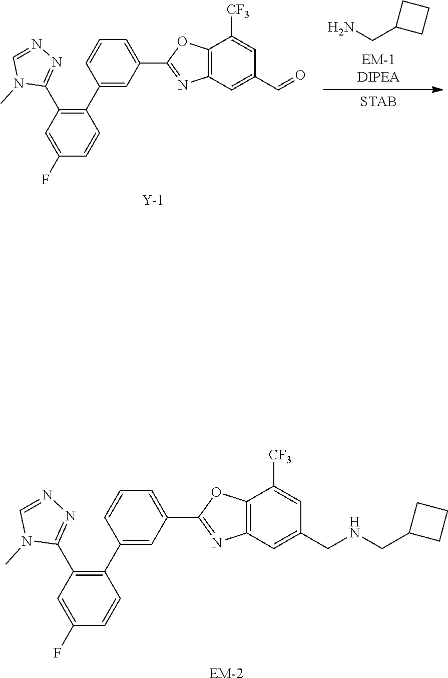

A solution of intermediate (Y-1) (31.0 mg, 90% Wt, 1 Eq, 59.8 μmol), DIPEA (23.2 mg, 31.3 μL, 3.0 Eq, 179 μmol) and cyclobutylmethanamine (EM-1) (10.2 mg, 2.0 Eq, 120 μmol) in CHCl₃ (3 mL) was stirred for 1 h at rt. NaBH(OAc)₃ (38.0 mg, 3.0 Eq, 179 μmol) was added and the reaction mixture was stirred at rt overnight. The reaction mixture was diluted with EtOAc (5 mL) and washed with a sat. aq sol. of NaHCO₃ (5 mL) and brine (5 mL). The organic layer was extracted, dried (MgSO₄), and concentrated in vacuo. The crude product was purified by chromatography on silica gel (12 g cartridge, 0-10% MeOH/DCM) to afford the title compound (EM-2) (18.6 mg, 33 μmol, 56%, 96% Purity) as a white solid. m/z 536.2 (M+H)⁺ (ES+). ¹H NMR (400 MHz, DMSO) δ 8.42 (s, 1H), 8.12 (dt, J=7.9, 1.4 Hz, 1H), 8.05 (s, 1H), 7.90 (t, J=1.8 Hz, 1H), 7.80-7.74 (m, 2H), 7.64-7.52 (m, 3H), 7.39-7.34 (m, 1H), 3.86 (s, 2H), 3.20 (s, 3H), 2.45-2.36 (m, 1H), 2.05-1.94 (m, 2H), 1.87-1.72 (m, 2H), 1.69-1.58 (m, 2H). CH₂ masked by DMSO peak, and one exchangeable proton not observed.

### Example 211: (R)—N-((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)-1-methoxypropan-2-amine (EN-2)

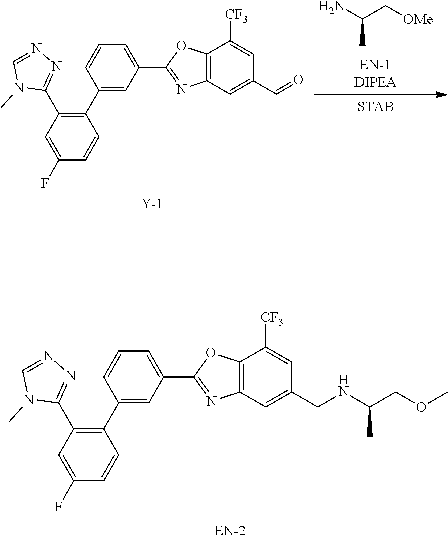

A solution of intermediate (Y-1) (30 mg, 90% Wt, 1 Eq, 58 μmol), DIPEA (22 mg, 30 μL, 3.0 Eq, 0.17 mmol) and (R)-1-methoxypropan-2-amine, HCl (EN-1) (15 mg, 2 Eq, 0.12 mmol) in CHCl₃ (3 mL) was stirred for 1 h at rt. NaBH(OAc)₃ (37 mg, 3.0 Eq, 0.17 mmol) was added and the reaction mixture was stirred at rt overnight. The reaction mixture was diluted with EtOAc (5 mL) and washed with a sat. aq sol. of NaHCO₃ (5 mL) and brine (5 mL). The organic layer was extracted, dried (MgSO₄), and concentrated in vacuo. The crude product was purified by chromatography on silica gel (12 g cartridge, 0-10% MeOH/DCM) to afford 90% pure product as a white solid. The crude product was purified by preparative HPLC (Waters, Basic (0.1% ammonia), Basic, Waters X-Bridge BEH C18 ODB, 5 μm, 30×100 mm column 35-100% MeCN in water) to afford the title compound (EN-2) (12 mg, 21 μmol, 37%, 96% Purity) as a white solid. m/z 540.0 (M+H)⁺ (ES+). ¹H NMR (400 MHz, MeOD) δ 8.41 (s, 1H), 8.24 (dt, J=7.9, 1.4 Hz, 1H), 8.09 (t, J=1.8 Hz, 1H), 8.03 (s, 1H), 7.81 (dd, J=8.6, 5.5 Hz, 1H), 7.79 (s, 1H), 7.63-7.55 (m, 2H), 7.52-7.44 (m, 2H), 4.08 (d, J=13.5 Hz, 1H), 3.98 (d, J=13.6 Hz, 1H), 3.40 (dd, J=9.5, 4.7 Hz, 1H), 3.38 (s, 3H), 3.27 (s, 3H), 3.01-2.90 (m, 1H), 1.13 (d, J=6.4 Hz, 3H). 2H under DMSO.

### Example 212: N-((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)-1-methoxy-2-methylpropan-2-amine (EO-2)

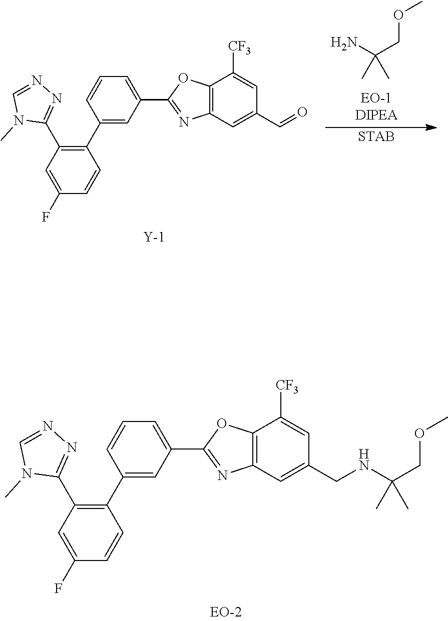

A solution of intermediate (Y-1) (31.0 mg, 90% Wt, 1 Eq, 59.8 μmol), DIPEA (23.2 mg, 31.3 μL, 3.0 Eq, 179 μmol) and 1-methoxy-2-methylpropan-2-amine (EO-1) (12.3 mg, 12 μL, 2.0 Eq, 120 μmol) in CHCl₃ (3 mL) was stirred for 1 h at rt. NaBH(OAc)₃ (38.0 mg, 3.0 Eq, 179 μmol) was added and the reaction mixture was stirred at rt overnight. The reaction mixture was diluted with EtOAc (5 mL) and washed with a sat. aq sol. of NaHCO₃ (5 mL) and brine (5 mL). The organic layer was extracted, dried (MgSO₄), and concentrated in vacuo. The crude product was purified by chromatography on silica gel (12 g cartridge, 0-10% MeOH/DCM) followed by purification by preparative HPLC (Waters, Basic (0.1% ammonia), Basic, Waters X-Bridge Prep-C18, 5 μm, 30×100 mm column, 0-100% MeCN in water) to afford the title compound (EO-2) (3.0 mg, 5.3 μmol, 8.9%, 98% Purity) as a white solid. m/z 554.4 (M+H)⁺ (ES+). ¹H NMR (400 MHz, MeOD) δ 8.41 (s, 1H), 8.27-8.22 (m, 1H), 8.12-8.08 (m, 1H), 8.02 (s, 1H), 7.84-7.79 (m, 1H), 7.77 (s, 1H), 7.64-7.55 (m, 2H), 7.52-7.43 (m, 2H), 3.92 (s, 2H), 3.44 (s, 3H), 3.27 (s, 3H), 1.21 (s, 6H). CH₂ masked by solvent peak and one exchangeable proton not observed.

### Example 213: N-((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)propan-2-amine (EP-2)

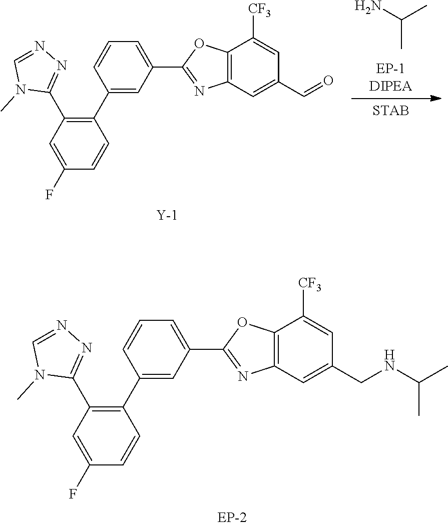

A solution of intermediate (Y-1) (31.0 mg, 90% Wt, 1 Eq, 59.8 μmol), DIPEA (23.2 mg, 31.3 μL, 3.0 Eq, 179 μmol) and propan-2-amine (EP-1) (7.07 mg, 10.3 μL, 2.0 Eq, 120 μmol) in CHCl₃ (3 mL) was stirred for 1 h at rt. NaBH(OAc)₃ (38.0 mg, 3.0 Eq, 179 μmol) was added and the reaction mixture was stirred at rt overnight. The reaction mixture was diluted with EtOAc (5 mL) and washed with a sat. aq sol. of NaHCO₃ (5 mL) and brine (5 mL). The organic layer was extracted, dried (MgSO₄), and concentrated in vacuo. The crude product was purified by chromatography on silica gel (12 g cartridge, 0-10% MeOH/DCM) to afford the title compound (EP-2) (13 mg, 26 μmol, 43%) as a white solid. m/z 510.2 (M+H)⁺ (ES+). ¹H NMR (400 MHz, DMSO) δ 8.42 (s, 1H), 8.12 (dt, J=7.8, 1.4 Hz, 1H), 8.07 (s, 1H), 7.90 (t, J=1.8 Hz, 1H), 7.79-7.75 (m, 2H), 7.64-7.53 (m, 3H), 7.40-7.36 (m, 1H), 3.88 (s, 2H), 3.20 (s, 3H), 2.71 (p, J=6.3 Hz, 1H), 1.02 (d, J=6.3 Hz, 6H). One exchangeable proton not observed.

### Example 214: (1S,2R)-2-(((7-Bromo-2-(4′-fluoro-2′-(4-methyl-4H-1,2,4-triazol-3-yl)-[1,1′-biphenyl]-3-yl)benzo[d]oxazol-5-yl)methyl)amino)cyclopentan-1-ol (EQ-4)

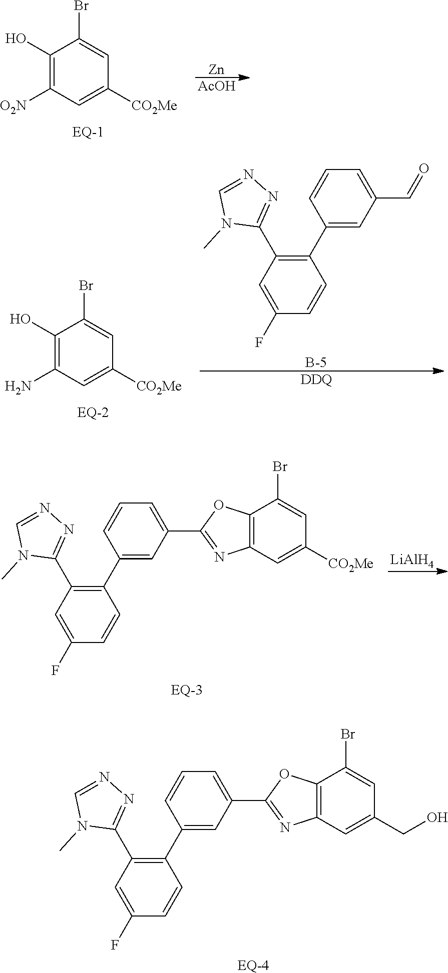

Step 1: Methyl 3-amino-5-bromo-4-hydroxybenzoate (EQ-2). To a stirred solution of 3-Bromo-4-hydroxy-5-nitro-benzoic acid methyl ester (EQ-1) (307.6 mg, 1 Eq, 1.114 mmol) and zinc (90.2 mg, 1.24 Eq, 1.38 mmol) in dry MeOH (8.0 mL) at rt was added AcOH (2.1 g, 2.0 mL, 31 Eq, 35 mmol). The reaction mixture was stirred at rt for 3 h. Zinc (218.0 mg, 2.992 Eq, 3.334 mmol) was added and the resulting mixture was stirred at rt overnight. The reaction mixture was filtered through a short pad of silica gel and washed with EtOAc. The filtrate was diluted with water (10 mL) and brine (10 mL), extracted with EtOAc (3×25 mL). The combined organic layers were collected, washed with sat. NaHCO₃, dried (Na₂SO₄), filtered and concentrated in vacuo. The crude product was purified by chromatography on silica gel (12 g cartridge, 0-10% MeOH/DCM) to afford the sub-title compound (EQ-2) (113.3 mg, 0.32 mmol, 29%, 69% Purity) as a pale brown solid. m/z 246.0, 248.0 (M+H)⁺ (ES+). ¹H NMR (400 MHz, DMSO) δ 7.28 (d, J=2.1 Hz, 1H), 7.24 (d, J=2.1 Hz, 1H), 3.77 (s, 3H), 3.29 (s, 1H). 2× Exchangeable protons not observed.

Step 2: Methyl 7-bromo-2-(4′-fluoro-2′-(4-methyl-4H-1,2,4-triazol-3-yl)-[1,1′-biphenyl]-3-yl)benzo[d]oxazole-5-carboxylate (EQ-3). To a solution of intermediate (B-5) (116.2 mg, 77% Wt, 1.001 Eq, 318.1 μmol) in MeOH (5.0 mL) was added the product from step 1 above (EQ-2) (113.3 mg, 69% Wt, 1 Eq, 317.7 μmol). The resulting mixture was stirred at 45° C. overnight. The reaction mixture was cooled to rt and the volatiles were removed in vacuo. The residue was re-dissolved in DCM (5.0 mL). DDQ (84.7 mg, 98% Wt, 1.15 Eq, 366 μmol) was added and the resulting mixture was stirred at rt for 1 h. The reaction mixture was diluted with DCM (10 mL), washed with sat. NaHCO₃ (3×10 mL) and brine. The organic layers were collected, dried (Na₂SO₄), filtered and concentrated in vacuo. The crude product was purified by chromatography on silica gel (12 g cartridge, 0-10% MeOH/DCM) to afford the sub-title compound (EQ-3) (209.0 mg, 0.32 mmol, 100%, 77% Purity) as a brown solid. m/z 507.1, 509.0 (M+H)⁺ (ES+). ¹H NMR (400 MHz, DMSO) δ 8.45 (s, 1H), 8.33 (d, J=1.5 Hz, 1H), 8.19 (d, J=1.4 Hz, 1H), 8.15 (dt, J=7.8, 1.4 Hz, 1H), 7.94 (t, J=1.8 Hz, 1H), 7.79 (dd, J=8.6, 5.7 Hz, 1H), 7.64-7.49 (m, 3H), 7.37 (dt, J=7.8, 1.5 Hz, 1H), 3.92 (s, 3H), 3.22 (s, 3H).

Step 3: (7-Bromo-2-(4′-fluoro-2′-(4-methyl-4H-1,2,4-triazol-3-yl)-[1,1′-biphenyl]-3-yl)benzo[d]oxazol-5-yl)methanol (EQ-4). To a solution of the product from step 2 above (EQ-3) (209.0 mg, 77% Wt, 1 Eq, 317.2 μmol) in THF (3.0 mL) was added LiAlH₄ in THF (12.1 mg, 320 μL, 1 molar, 1.01 Eq, 320 μmol) dropwise at 0° C. This mixture was stirred at 0° C. for 1 h. The mixture was quenched with H₂O (0.5 mL) and NaOH (2 M, 0.5 mL) at 0° C. MgSO₄ was added. The mixture was warmed up to rt, stirred for 15 min, filtered and the solvent was removed in vacuo. The crude product was purified by chromatography on silica gel (12 g cartridge, 0-10% MeOH/DCM) to afford impure product. The crude product was purified by preparative HPLC (Waters, acidic (0.1% formic acid), acidic, Waters X-Select CSH C18 ODB, 5 μm, 30×100 mm column, 25-100% MeCN in water) to afford the title compound (EQ-4) (6.0 mg, 12 μmol, 90%, 96% Purity) as a white solid. m/z 479.0, 481.0 (M+H)⁺ (ES+). ¹H NMR (400 MHz, DMSO) δ 8.44 (s, 1H), 8.15-8.10 (m, 1H), 7.93 (t, J=1.8 Hz, 1H), 7.79 (dd, J=8.6, 5.6 Hz, 1H), 7.74-7.70 (m, 1H), 7.64-7.52 (m, 4H), 7.36-7.31 (m, 1H), 5.45 (t, J=5.8 Hz, 1H), 4.62 (d, J=5.9 Hz, 2H), 3.21 (s, 3H).

### Example 215: (1S,2R)-2-(((7-Bromo-2-(4′-fluoro-2′-(4-methyl-4H-1,2,4-triazol-3-yl)-[1,1′-biphenyl]-3-yl)benzo[d]oxazol-5-yl)methyl)amino)cyclopentan-1-ol (ER-2)

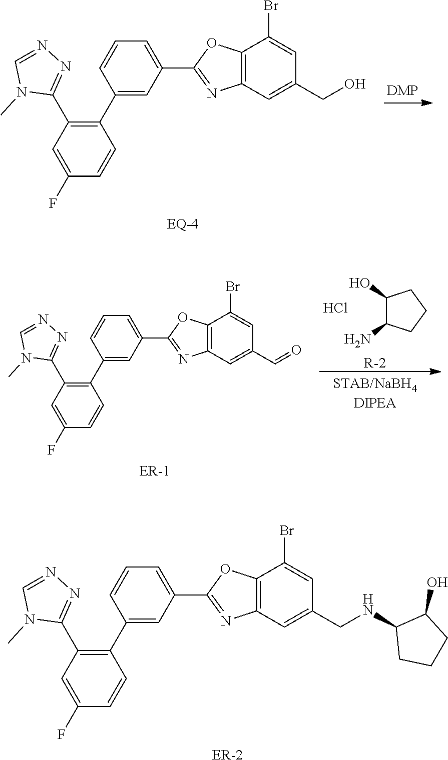

Step 1: 7-Bromo-2-(4′-fluoro-2′-(4-methyl-4H-1,2,4-triazol-3-yl)-[1,1′-biphenyl]-3-yl)benzo[d]oxazole-5-carbaldehyde (ER-1). To a stirred solution of compound (EQ-4) (54.2 mg, 81% Wt, 1 Eq, 91.6 μmol) in dry DCM (3.0 mL) at rt was added Dess-Martin periodinane (98.1 mg, 2.53 Eq, 231 μmol). The reaction mixture was stirred at rt for 1 h. MeCN (2.0 mL) and THF (2.0 mL) were added, and the reaction was stirred at rt for another 1 h. More Dess-Martin periodinane (102.6 mg, 2.64 Eq, 241.9 μmol) was added and the reaction mixture was stirred at rt for 2 h. Another batch of Dess-Martin periodinane (97.8 mg, 2.52 Eq, 231 μmol) was added and the reaction was stirred at rt overnight. The reaction mixture was diluted with DCM (5 mL), washed with sat. NaHCO₃ (5 mL) and brine (5 mL). The organic layer was dried (Na₂SO₄), filtered and concentrated in vacuo. The crude product was purified by chromatography on silica gel (12 g cartridge, 0-10% MeOH/DCM) to afford the sub-title compound (ER-1) (58.5 mg, 91 μmol, 99%, 74% Purity) as a pale-yellow solid. m/z 477.1, 479.0 (M+H)⁺ (ES+). ¹H NMR (400 MHz, DMSO) δ 10.08 (s, 1H), 8.45 (s, 1H), 8.37 (d, J=1.4 Hz, 1H), 8.21 (d, J=1.3 Hz, 1H), 8.18-8.15 (m, 1H), 7.96 (t, J=1.8 Hz, 1H), 7.79 (dd, J=8.6, 5.6 Hz, 1H), 7.65-7.53 (m, 3H), 7.41-7.36 (m, 1H), 3.23 (s, 3H).

Step 2: (1S,2R)-2-(((7-Bromo-2-(4′-fluoro-2′-(4-methyl-4H-1,2,4-triazol-3-yl)-[1,1′-biphenyl]-3-yl)benzo[d]oxazol-5-yl)methyl)amino)cyclopentan-1-ol (ER-2). A solution of the product from step 1 above (ER-1) (58.5 mg, 74% Wt, 1 Eq, 90.7 μmol), (1S,2R)-2-aminocyclopentan-1-ol, HCl (R-2) (25.2 mg, 2.02 Eq, 183 μmol) and DIPEA (59 mg, 80 μL, 5.1 Eq, 0.46 mmol) in CHCl₃ (3.0 mL) was stirred 1 h at rt. NaBH(OAc)₃ (57.1 mg, 97% Wt, 2.88 Eq, 261 μmol) was added and the reaction mixture was stirred at rt overnight. NaBH₄ (3.43 mg, 1 Eq, 90.7 μmol) was added and the reaction mixture was stirred at rt for 2 h. The reaction mixture was diluted with EtOAc (2 mL), washed with sat. NaHCO₃ (2 mL) and brine (2 mL), dried (Na₂SO₄), filtered and concentrated in vacuo. The crude product was purified by chromatography on silica gel (4 g cartridge, 0-20% MeOH/DCM) to afford the title compound (ER-2) (5.2 mg, 8.9 μmol, 9.8%, 96% Purity) as an off-white solid. m/z 562.1, 564.1 (M+H)⁺ (ES+). ¹H NMR (400 MHz, MeOD) δ 8.40 (s, 1H), 8.22-8.19 (m, 1H), 8.05 (s, 1H), 7.82-7.74 (m, 2H), 7.68 (s, 1H), 7.61-7.52 (m, 2H), 7.49-7.41 (m, 2H), 4.24-4.13 (m, 1H), 4.06-4.00 (m, 1H), 3.98-3.92 (m, 1H), 3.24 (s, 3H), 3.08-3.01 (m, 1H), 1.97-1.72 (m, 4H), 1.63-1.52 (m, 2H). 2× Exchangeable protons not observed.

### Example 216: (1S,2R)-2-(((7-Cyclopropyl-2-(4′-fluoro-2′-(4-methyl-4H-1,2,4-triazol-3-yl)-[1,1′-biphenyl]-3-yl)benzo[d]oxazol-5-yl)methyl)amino)cyclopentan-1-ol (ES-6)

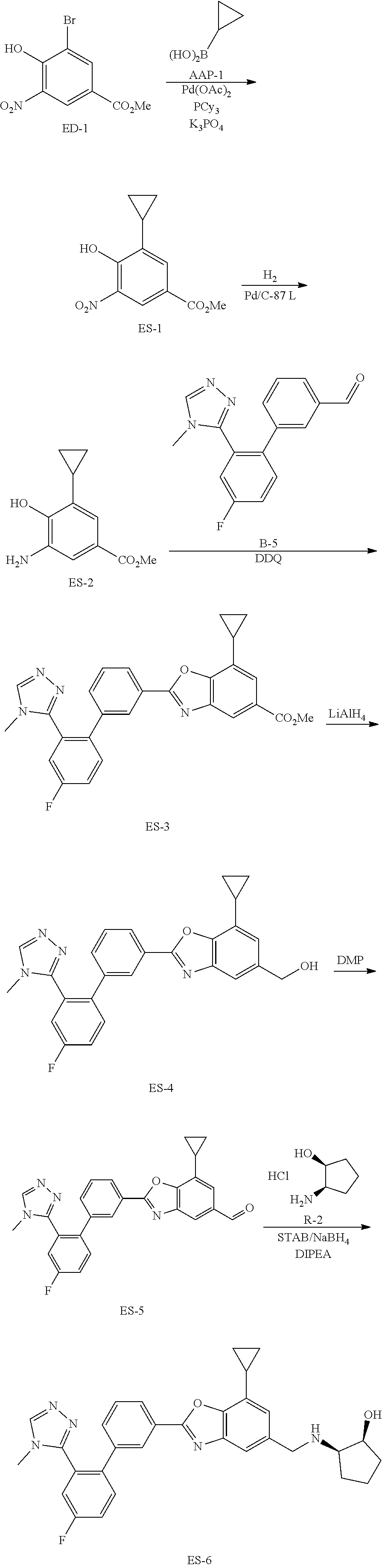

Step 1: Methyl 3-cyclopropyl-4-hydroxy-5-nitrobenzoate (ES-1). A stirred solution of 3-Bromo-4-hydroxy-5-nitro-benzoic acid, methyl ester (EQ-1) (708.5 mg, 1 Eq, 2.567 mmol), cyclopropaneboronic acid (AAP-1) (243.1 mg, 1.103 Eq, 2.830 mmol), K₃PO₄ (1.62 g, 632 μL, 2.97 Eq, 7.63 mmol), tricyclohexylphosphine (284.2 mg, 317 μL, 0.3948 Eq, 1.013 mmol) and Pd(OAc)₂ (58.7 mg, 0.102 Eq, 261 μmol) in toluene (10 mL) and water (0.25 mL) was stirred at 100° C. under a nitrogen atmosphere overnight. The reaction mixture was cooled to rt, diluted with brine (50 mL) and transferred into a separating funnel. The layer was extracted with EtOAc (3×50 mL). The combined organic layers were collected, dried (Na₂SO₄), filtered and concentrated in vacuo. The crude product was purified by chromatography on silica gel (40 g cartridge, 0-10% MeOH/DCM) to afford the sub-title compound (ES-1) (86.0 mg, 0.35 mmol, 14%, 97% Purity) as a pale brown solid. ¹H NMR (400 MHz, DMSO) δ 11.20 (s, 1H), 8.29 (d, J=2.1 Hz, 1H), 7.65 (d, J=2.1 Hz, 1H), 3.84 (s, 3H), 2.18 (tt, J=8.5, 5.3 Hz, 1H), 1.07-0.96 (m, 2H), 0.75-0.67 (m, 2H).

Step 2: Methyl 3-amino-5-cyclopropyl-4-hydroxybenzoate (ES-2). To a solution of the product from step 1 above (ES-1) (86.0 mg, 97% Wt, 1 Eq, 352 μmol) in MeOH (5.0 mL) was added Pd/C 87 L (37.2 mg, 5% Wt, 0.0497 Eq, 17.5 μmol). The reaction mixture was stirred at rt with 5 atm of H₂ for 2 h. The mixture was filtered, and the volatiles were removed in vacuo to afford the sub-title compound (ES-2) (61.4 mg, 0.29 mmol, 83%, 98% Purity) as a pale brown solid. m/z 208.1 (M+H)⁺ (ES+). ¹H NMR (400 MHz, DMSO) δ 7.12 (d, J=2.1 Hz, 1H), 6.72 (d, J=2.2 Hz, 1H), 3.73 (s, 3H), 2.08-1.99 (m, 1H), 0.92-0.84 (m, 2H), 0.55-0.48 (m, 2H). 3× exchangeable protons not observed.

Step 3: Methyl 7-cyclopropyl-2-(4′-fluoro-2′-(4-methyl-4H-1,2,4-triazol-3-yl)-[1,1′-biphenyl]-3-yl)benzo[d]oxazole-5-carboxylate (ES-3). A solution of intermediate (B-5) (146.7 mg, 77% Wt, 1.38 Eq, 401.6 μmol) and the product from step 2 above (ES-2) (88.4 mg, 1.38 Eq, 399 μmol) in MeOH (5.0 mL) was heated at 45° C. overnight. The reaction mixture was cooled to rt and the volatiles were removed in vacuo. The residue was re-dissolved in DCM (5 mL). DDQ (105.5 mg, 98% Wt, 1.57 Eq, 455.5 μmol) was added and the resulting mixture was stirred at rt for 1 h. The reaction mixture was diluted with DCM (10 mL), washed with sat. NaHCO₃ (3×10 mL) and brine. The organic layers were collected, dried (Na₂SO₄), filtered and concentrated in vacuo. The crude product was purified by chromatography on silica gel (12 g cartridge, 0-10% MeOH/DCM) to afford the sub-title compound (ES-3) (233.3 mg, 0.30 mmol, 75%, 60% Purity) as a brown solid, containing 27 wt % DCM and 13 wt % unreacted (B-5). m/z 469.2 (M+H)⁺ (ES+). ¹H NMR (400 MHz, DMSO) δ 8.43 (s, 1H), 8.16 (dt, J=7.9, 1.3 Hz, 1H), 8.11 (d, J=1.6 Hz, 1H), 7.94 (t, J=1.8 Hz, 1H), 7.79 (dd, J=8.6, 5.6 Hz, 1H), 7.68 (d, J=1.6 Hz, 1H), 7.64-7.49 (m, 3H), 7.38 (dt, J=8.0, 1.3 Hz, 1H), 3.89 (s, 3H), 3.17 (s, 3H), 2.43-2.35 (m, 1H), 1.20-1.13 (m, 2H), 1.11-1.04 (m, 2H).

Step 4: (7-Cyclopropyl-2-(4′-fluoro-2′-(4-methyl-4H-1,2,4-triazol-3-yl)-[1,1′-biphenyl]-3-yl)benzo[d]oxazol-5-yl)methanol (ES-4). To a solution of the product from step 3 above (ES-3) (233.3 mg, 60% Wt, 1 Eq, 316.4 μmol) in THF (3.0 mL) was added LiAlH₄ in THF (12.01 mg, 316.4 μL, 1 molar, 1 Eq, 316.4 μmol) dropwise at 0° C. This mixture was stirred at 0° C. for 1 h. The mixture was quenched with H₂O (0.5 mL) and NaOH (2 M, 0.5 mL) at 0° C. MgSO₄ was added. The mixture was warmed up to rt, stirred for 15 min, filtered and the solvent was removed in vacuo. The crude product was purified by chromatography on silica gel (12 g cartridge, 0-10% MeOH/DCM) to afford the sub-title compound (ES-4) (185.9 mg, 0.24 mmol, 77%, 58% Purity) as a yellow oil, containing 28 wt % DCM and 14 wt % reduced form of (B-5) carried through from previous step. m/z 441.2 (M+H)⁺ (ES+). ¹H NMR (400 MHz, DMSO) δ 8.43 (s, 1H), 8.13 (dt, J=7.9, 1.4 Hz, 1H), 7.92 (t, J=1.8 Hz, 1H), 7.79 (dd, J=8.6, 5.6 Hz, 1H), 7.66-7.50 (m, 3H), 7.48 (d, J=1.5 Hz, 1H), 7.33 (dt, J=7.9, 1.4 Hz, 1H), 7.03 (d, J=1.5 Hz, 1H), 5.25 (t, J=5.8 Hz, 1H), 4.56 (d, J=5.8 Hz, 2H), 3.17 (s, 3H), 2.29 (td, J=8.5, 4.3 Hz, 1H), 1.14-1.07 (m, 2H), 1.02-0.96 (m, 2H).

Step 5: 7-Cyclopropyl-2-(4′-fluoro-2′-(4-methyl-4H-1,2,4-triazol-3-yl)-[1,1′-biphenyl]-3-yl)benzo[d]oxazole-5-carbaldehyde (ES-5). To a stirred solution of the product from step 4 above (ES-4) (185.9 mg, 58% Wt, 1 Eq, 244.8 μmol) in dry DCM (3.0 mL) at rt was added Dess-Martin periodinane (136.3 mg, 1.313 Eq, 321.4 μmol). The reaction mixture was stirred at rt for 1 h. Dess-Martin periodinane (139.4 mg, 1.343 Eq, 328.7 μmol) was added and the resulting mixture was stirred at rt for 2 h. Dess-Martin periodinane (141.3 mg, 1.361 Eq, 333.1 μmol) was added and the reaction mixture was stirred at rt for 1 h. The reaction mixture was diluted with DCM (5 mL), washed with sat. NaHCO₃ (5 mL) and brine (5 mL). The organic layer was dried (Na₂SO₄), filtered and concentrated in vacuo. The crude product was purified by chromatography on silica gel (12 g cartridge, 0-10% MeOH/DCM) to afford the sub-title compound (ES-5) (120.4 mg, 0.21 mmol, 84%, 75% Purity) as a white solid, containing 15 wt % (B-5). m/z 439.1 (M+H)⁺ (ES+). ¹H NMR (400 MHz, DMSO) δ 10.06 (s, 1H), 8.44 (s, 1H), 8.18 (dt, J=7.9, 1.4 Hz, 1H), 8.12 (d, J=1.5 Hz, 1H), 7.96 (t, J=1.8 Hz, 1H), 7.79 (dd, J=8.6, 5.6 Hz, 1H), 7.68-7.65 (m, 1H), 7.65-7.60 (m, 1H), 7.60-7.56 (m, 1H), 7.56-7.50 (m, 1H), 7.38 (dt, J=7.8, 1.4 Hz, 1H), 3.17 (s, 3H), 2.45-2.38 (m, 1H), 1.23-1.16 (m, 2H), 1.13-1.08 (m, 2H).

Step 6: (1S,2R)-2-(((7-Cyclopropyl-2-(4′-fluoro-2′-(4-methyl-4H-1,2,4-triazol-3-yl)-[1,1′-biphenyl]-3-yl)benzo[d]oxazol-5-yl)methyl)amino)cyclopentan-1-ol (ES-6). A solution of the product from step 5 above (ES-5) (31.3 mg, 75% Wt, 1 Eq, 53.5 μmol), (1S,2R)-2-aminocyclopentan-1-ol, HCl (R-2) (17.7 mg, 2.40 Eq, 129 μmol) and DIPEA (37 mg, 50 μL, 5.4 Eq, 0.29 mmol) in CHCl₃ (3.0 mL) was stirred 1 h at rt. NaBH(OAc)₃ (45.0 mg, 97% Wt, 3.85 Eq, 206 μmol) was added and the reaction mixture was stirred at rt overnight. The reaction mixture was diluted with EtOAc (2 mL), washed with sat. NaHCO₃ (2 mL) and brine (2 mL), dried (Na₂SO₄), filtered and concentrated in vacuo. The crude product was purified by chromatography on silica gel (4 g cartridge, 0-20% MeOH/DCM) to afford the desired product at ˜90% purity. The crude product was further purified by preparative HPLC (Waters, Basic (0.1% ammonia), Basic, Waters X-Bridge BEH C18 ODB, 5 μm, 30×100 mm column 30-100% MeCN in water) to afford the title compound (ES-6) (4.7 mg, 8.9 μmol, 17%, 99% Purity) as a white solid. m/z 524.3 (M+H)⁺ (ES+), ¹H NMR (400 MHz, MeOD) δ 8.38 (s, 1H), 8.19 (dt, J=7.9, 1.4 Hz, 1H), 8.02 (t, J=1.8 Hz, 1H), 7.78 (dd, J=8.6, 5.5 Hz, 1H), 7.59-7.49 (m, 3H), 7.49-7.39 (m, 2H), 7.09 (d, J=1.6 Hz, 1H), 4.16 (td, J=4.3, 2.2 Hz, 1H), 3.93 (d, J=12.8 Hz, 1H), 3.84 (d, J=12.8 Hz, 1H), 3.18 (s, 3H), 2.96 (ddd, J=9.2, 7.5, 4.2 Hz, 1H), 2.33 (tt, J=8.4, 5.2 Hz, 1H), 1.96-1.67 (m, 4H), 1.61-1.47 (m, 2H), 1.19-1.11 (m, 2H), 1.06-0.99 (m, 2H). 2× Exchangeable protons not observed.

### Example 217: (1S,2R)-2-(((2-(4′-Fluoro-2′-(4-methyl-4H-1,2,4-triazol-3-yl)-[1,1′-biphenyl]-3-yl)benzo[d]thiazol-5-yl)methyl)amino)cyclopentan-1-ol (ET-8)

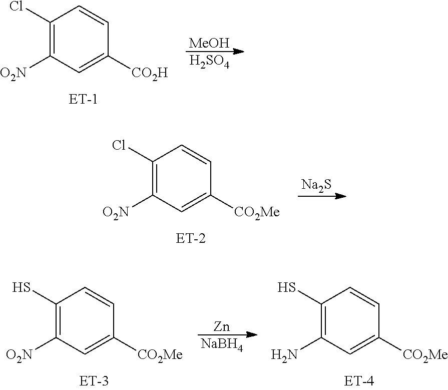

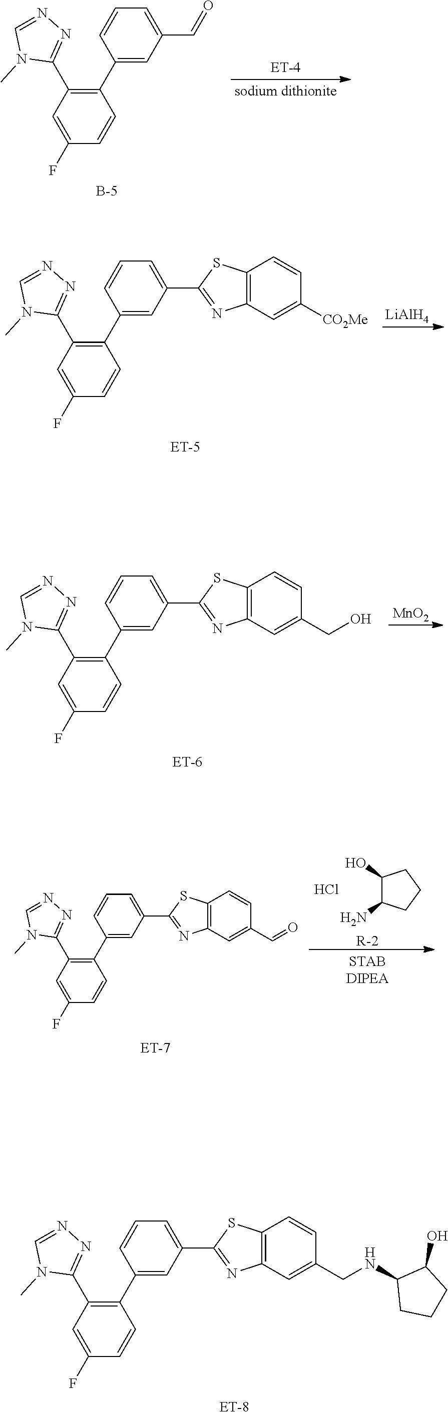

Step 1: Methyl 4-chloro-3-nitrobenzoate (ET-2). Sulfuric acid (0.7 g, 0.4 mL, 0.7 Eq, 7 mmol) was added to 4-chloro-3-nitrobenzoic acid (ET-1) (2.00 g, 1 Eq, 9.92 mmol) in MeOH (15 mL) and stirred at reflux overnight. The bulk solvent was removed in vacuo and the resultant residue dissolved in EtOAc (50 mL) and washed with sat. aq. NaHCO₃ (50 mL). The organic layer was evaporated to dryness to afford the sub-title compound (ET-2) (2.09 g, 9.6 mmol, 97%, 99% Purity) as a pale-yellow crystalline solid. ¹H NMR (400 MHz, DMSO-d6) δ 8.53 (d, J=2.0 Hz, 1H), 8.20 (dd, J=8.4, 2.1 Hz, 1H), 7.95 (d, J=8.4 Hz, 1H), 3.91 (s, 3H).

Step 2: Methyl 4-mercapto-3-nitrobenzoate (ET-3). Sodium sulfide (899 mg, 503 μL, 1.2 Eq, 11.5 mmol) was added to the product from step 1 above (ET-2) (2.09 g, 99% Wt, 1 Eq, 9.60 mmol) in DMF (20 mL) and stirred at rt for 16 h. Water (50 mL) was added to the reaction mixture which was then adjusted with 1 M aq. HCl to pH 5. The resultant suspension was filtered and the collected solid sonicated in 1:4 EtOAc-iso-hexane (20 mL) and filtered. The collected solid was dissolved in acetone and the resultant solution evaporated to dryness (removing residual water) to afford the sub-title compound (ET-3) (417 mg, 1.8 mmol, 18%, 90% Purity) as a pale yellow flocculent solid. ¹H NMR (400 MHz, DMSO-d6) δ 8.57 (d, J=1.9 Hz, 1H), 8.02 (dd, J=8.4, 1.9 Hz, 1H), 7.91 (d, J=8.4 Hz, 1H), 3.88 (s, 3H). ArSH signal not observed. m/z 212.1 (M−H)⁻ (ES−).

Step 3: Methyl 3-amino-4-mercaptobenzoate (ET-4). Zinc (1.38 g, 12 Eq, 21.1 mmol) was added portion-wise over 2 minutes to an ice-cold suspension of the product from step 2 above (ET-3) (417 mg, 90% Wt, 1 Eq, 1.76 mmol) in a mixture of AcOH (8 mL) and aq. cone. HCl (4 mL, 37% Wt, 1 Eq, 1.76 mmol). The resultant slurry was stirred at reflux until the reaction turned colourless. The reaction mixture was left to stand at rt over the weekend. The reaction mixture was treated with a solution of NaOAc (3 g) in water (30 mL). The solid was collected and dried by repeated co-evaporation with EtOH in vacuo. The resultant white solid was left to stand at rt then suspended in dry MeOH (10 ml) and treated with NaBH₄ (266 mg, 249 μL, 4 Eq, 7.04 mmol) at 0° C. After 30 minutes, the reaction was concentrated, diluted with water and acidified with aqueous HCl. The product was extracted with EtOAc (2×30 mL) and combined organic extracts concentrated in vacuo to a white solid. The crude product was purified by chromatography on silica gel (12 g cartridge, 0-100% DCM in iso-hexane) to afford the sub-title compound (ET-4) (63 mg, 0.33 mmol, 19%, 95% Purity) as a pale-yellow solid. m/z 182.3 (M−H)⁻ (ES−). ¹H NMR (400 MHz, DMSO) δ 7.34 (d, J=1.9 Hz, 1H), 7.26 (d, J=8.0 Hz, 1H), 7.10 (dd, J=8.1, 1.9 Hz, 1H), 5.77 (d, J=15.2 Hz, 3H), 3.78 (s, 3H).

Step 4: Methyl 2-(4′-fluoro-2′-(4-methyl-4H-1,2,4-triazol-3-yl)-[1,1′-biphenyl]-3-yl)benzo[d]thiazole-5-carboxylate (ET-5). Sodium dithionite (114 mg, 2.0 Eq, 653 μmol) in water (1 mL) was added to a solution of the product from step 3 above (ET-4) (63.0 mg, 95% Wt, 1 Eq, 327 μmol) and intermediate (B-5) (115 mg, 80% Wt, 1 Eq, 327 μmol) in EtOH (2 mL) and stirred at 80° C. for 16 h. The reaction was allowed to cool to rt and the bulk solvent removed in vacuo. The resultant residue was partitioned between DCM (10 mL) and water (10 ml). The layers were separated, and the organic layer washed with aq. 1 M HCl (10 mL), dried (phase separator) and concentrated in vacuo to afford the sub-title compound (ET-5) (131 mg, 0.27 mmol, 83%, 92% Purity) as a colourless foam. m/z 445.2 (M+H)⁺ (ES+). ¹H NMR (400 MHz, DMSO) δ 8.55 (d, J=1.6 Hz, 1H), 8.47 (s, 1H), 8.32 (d, J=8.5 Hz, 1H), 8.07 (dt, J=8.0, 1.3 Hz, 1H), 8.03 (dd, J=8.4, 1.6 Hz, 1H), 7.78 (dd, J=6.3, 2.4 Hz, 2H), 7.61 (td, J=8.6, 2.8 Hz, 1H), 7.58-7.53 (m, 2H), 7.37 (dt, J=8.0, 1.3 Hz, 1H), 3.92 (s, 3H), 3.18 (s, 3H).

Step 5: (2-(4′-Fluoro-2′-(4-methyl-4H-1,2,4-triazol-3-yl)-[1,1′-biphenyl]-3-yl)benzo[d]thiazol-5-yl)methanol (ET-6). LiAlH₄ (in THF) (10.1 mg, 265 μL, 1 molar, 1.0 Eq, 265 μmol) was added to a stirred solution of the product from step 4 above (ET-5) (131 mg, 90% Wt, 1 Eq, 265 μmol) in THF (4 mL) at 0° C. The reaction mixture was stirred at 0° C. for 60 min. The reaction mixture was diluted with THF (5 ml) before being vigorously stirred with Na₂SO₄ decahydrate (c. 600 mg) added portion-wise at 0° C. The reaction mixture was partitioned between EtOAc (20 mL) and water (20 mL). The biphasic mixture was separated, and the aqueous layer was re-extracted with EtOAc (20 mL). The organic extracts were combined, dried (MgSO₄) and concentrated in vacuo to afford the sub-title compound (ET-6) (109 mg, 0.24 mmol, 91%, 92% Purity) as a pale-yellow foam. m/z 417.2 (M+H)⁺ (ES+). ¹H NMR (400 MHz, DMSO) δ 8.43 (s, 1H), 8.08 (d, J=8.3 Hz, 1H), 8.03 (dt, J=8.0, 1.3 Hz, 1H), 7.98 (d, J=1.5 Hz, 1H), 7.78 (dd, J=8.7, 5.7 Hz, 1H), 7.75 (t, J=1.8 Hz, 1H), 7.63-7.57 (m, 1H), 7.55-7.50 (m, 2H), 7.43 (dd, J=8.3, 1.6 Hz, 1H), 7.33 (dt, J=8.0, 1.3 Hz, 1H), 5.36 (t, J=5.8 Hz, 1H), 4.66 (d, J=5.7 Hz, 2H), 3.16 (s, 3H).

Step 6: 2-(4′-Fluoro-2′-(4-methyl-4H-1,2,4-triazol-3-yl)-[1,1′-biphenyl]-3-yl)benzo[d]thiazole-5-carbaldehyde (ET-7). The product from step 5 above (ET-6) (109 mg, 1.0 Eq, 241 μmol) and MnO₂ (628 mg, 30 Eq, 7.22 mmol) in CHCl₃ (10 mL) was stirred at 40° C. for 3 h. The reaction mixture was then left to stir at rt over the weekend. The reaction mixture was filtered through a small Celite plug (chased with 10 mL MeOH). The filtrate was evaporated to dryness to afford the sub-title compound (ET-7) (97 mg, 0.22 mmol, 91%, 94% Purity) as a pale beige foam. m/z 415.1 (M+H)⁺ (ES+). ¹H NMR (400 MHz, DMSO) δ 10.16 (s, 1H), 8.58 (d, J=1.5 Hz, 1H), 8.44 (s, 1H), 8.39 (d, J=8.3 Hz, 1H), 8.12-8.05 (m, 1H), 7.97 (dd, J=8.3, 1.5 Hz, 1H), 7.84-7.75 (m, 2H), 7.66-7.51 (m, 3H), 7.41-7.34 (m, 1H), 3.17 (s, 3H).

Step 7: (1S,2R)-2-(((2-(4′-Fluoro-2′-(4-methyl-4H-1,2,4-triazol-3-yl)-[1,1′-biphenyl]-3-yl)benzo[d]thiazol-5-yl)methyl)amino)cyclopentan-1-ol (ET-8). A mixture of the product from step 6 above (ET-7) (27 mg, 90% Wt, 1 Eq, 59 μmol), (1S,2R)-2-aminocyclopentan-1-ol, HCl (R-2) (16 mg, 2.0 Eq, 0.12 mmol) and DIPEA (45 mg, 61 μL, 6.0 Eq, 0.35 mmol) in CHCl₃ (2 mL) was stirred at rt for 5 min. NaBH(OAc)₃ (75 mg, 6.0 Eq, 0.35 mmol) was then added and the resultant suspension stirred at 40° C. overnight. More (1S,2R)-2-aminocyclopentan-1-ol, HCl (R-2) (16 mg, 2.0 Eq, 0.12 mmol), DIPEA (45 mg, 61 μL, 6.0 Eq, 0.35 mmol) and NaBH(OAc)₃ (75 mg, 6.0 Eq, 0.35 mmol) were added to the reaction mixture and stirring continued at 40° C. overnight. NaBH₄ (11 mg, 5.0 Eq, 0.29 mmol) was added and stirring continued at rt for 2 h. More NaBH₄ (11 mg, 5.0 Eq, 0.29 mmol) was added and stirring continued at rt overnight. The reaction mixture then was diluted with DCM (4 mL) and washed with sat. aq. NaHCO₃ (10 mL). The organic phase was separated and evaporated to afford the crude product. The crude product was purified by preparative HPLC (Waters, Basic (0.1% ammonia), Basic, Waters X-Bridge BEH C18 ODB, 5 μm, 30×100 mm column 25-100% MeCN in water) to afford the title compound (ET-8) (9 mg, 0.02 mmol, 30%, 97% Purity) as a colourless solid. m/z 500.2 (M+H)⁺ (ES+). ¹H NMR (400 MHz, DMSO) δ 8.43 (s, 1H), 8.09-7.99 (m, 3H), 7.82-7.72 (m, 2H), 7.60 (td, J=8.6, 2.9 Hz, 1H), 7.57-7.50 (m, 2H), 7.47 (dd, J=8.2, 1.6 Hz, 1H), 7.35-7.29 (m, 1H), 4.38 (s, 1H), 4.00-3.81 (m, 3H), 3.15 (s, 3H), 2.80 (td, J=7.4, 4.1 Hz, 1H), 1.76-1.51 (m, 4H), 1.49-1.34 (m, 2H). Missing proton under solvent peak.

### Example 218: (R)-1-(2-(4′-Fluoro-2′-(4-methyl-4H-1,2,4-triazol-3-yl)-[1,1′-biphenyl]-3-yl)-7-(trifluoromethyl)benzo[d]oxazol-5-yl)-N-((tetrahydrofuran-2-yl)methyl)methanamine (EU-2)

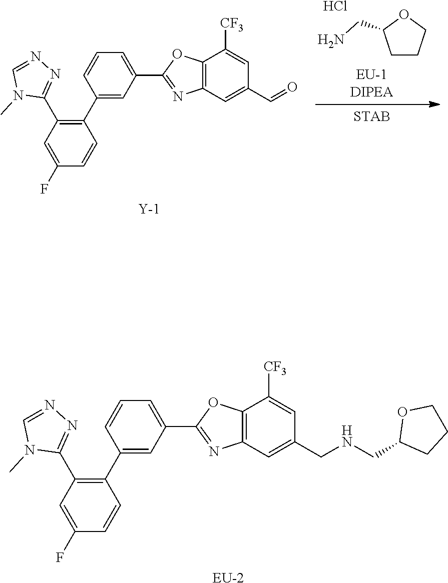

A solution of intermediate (Y-1) (30 mg, 90% Wt, 1 Eq, 58 μmol), DIPEA (22 mg, 30 μL, 3.0 Eq, 0.17 mmol) and (R)-(tetrahydrofuran-2-yl)methanamine, HCl (EU-1) (16 mg, 2 Eq, 0.12 mmol) in CHCl₃ (3 mL) was stirred for 1 h at rt. NaBH(OAc)₃ (37 mg, 3.0 Eq, 0.17 mmol) was added and the reaction mixture was stirred at rt overnight. The reaction mixture was diluted with EtOAc (5 mL) and washed with a sat. aq sol. of NaHCO₃ (5 mL) and brine (5 mL). The organic layer was extracted, dried (MgSO₄), and concentrated in vacuo. The crude product was purified by chromatography on silica gel (12 g cartridge, 0-10% MeOH/DCM) to afford the title compound (EU-2) (22.05 mg, 39 μmol, 68%, 98% Purity) as a white solid. m/z 552.2 (M+H)⁺ (ES+). ¹H NMR (400 MHz, DMSO) δ 8.42 (s, 1H), 8.13 (d, J=7.9 Hz, 1H), 8.06 (s, 1H), 7.91 (t, J=1.8 Hz, 1H), 7.80-7.75 (m, 2H), 7.64-7.53 (m, 3H), 7.37 (d, J=7.9 Hz, 1H), 3.91 (s, 2H), 3.89-3.84 (m, 1H), 3.75-3.68 (m, 1H), 3.63-3.56 (m, 1H), 3.20 (s, 3H), 1.95-1.85 (m, 1H), 1.85-1.74 (m, 2H), 1.60-1.47 (m, 1H). Two protons masked by DMSO peak on one exchangeable proton not observed.

### Example 219: (1S,2R)-2-(((7-(tert-Butyl)-2-(4′-fluoro-2′-(4-methyl-4H-1,2,4-triazol-3-yl)-[1,1′-biphenyl]-3-yl)benzo[d]oxazol-5-yl)methyl)amino)cyclopentan-1-ol (EV-9)

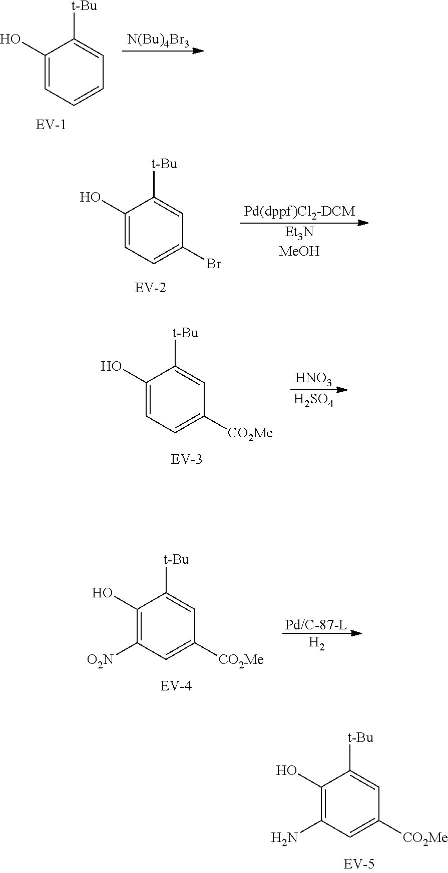

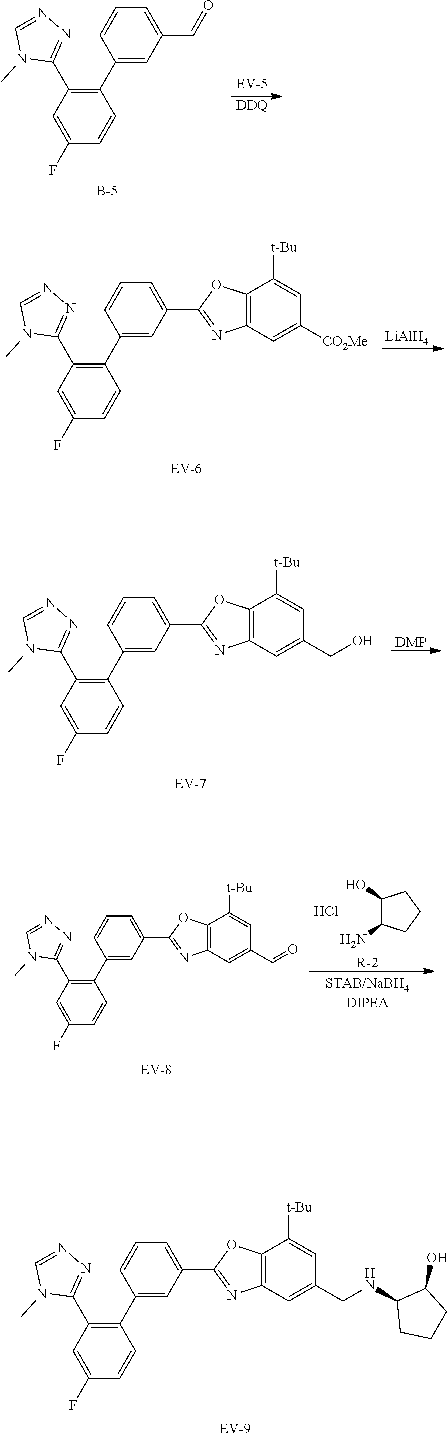

Step 1: 4-Bromo-2-(tert-butyl)phenol (EV-2). A solution of 2-(tert-butyl)phenol (EV-1) (1.00 g, 1 Eq, 6.66 mmol) in DCM (30 mL) and MeOH (20 mL) was treated with tetra-n-butylammonium tribromide (3.85 g, 1.2 Eq, 7.99 mmol). After 1 h, the mixture was concentrated in vacuo and the residue was taken up in Et₂O. The solution was washed with 1 N HCl (×2) and brine. Organics were dried, filtered and concentrated to afford the sub-title compound (EV-2) (1.52 g, 5.0 mmol, 75%, 75% Purity) as brown solid. m/z 227/229 (M−H)⁻ (ES−). ¹H NMR (400 MHz, DMSO) δ 9.66 (s, 1H), 7.19-7.13 (m, 2H), 6.74 (d, J=8.3 Hz, 1H), 1.32 (s, 9H).

Step 2: Methyl 3-(tert-butyl)-4-hydroxybenzoate (EV-3). The product from step 1 above (EV-2) (1.52 g, 1 Eq, 6.63 mmol), Pd(dppf)Cl₂-DCM complex (485 mg, 0.1 Eq, 663 μmol) and Et₃N (2.01 g, 2.77 mL, 3 Eq, 19.9 mmol) were mixed in MeOH (30 mL). The reaction vessel was charged with CO and then heated to 120° C. for 16 h. Reaction was cooled down, silica added (10 g) and volatiles evaporated. The crude product was purified by chromatography on silica gel (40 g cartridge, 0-50% EtOAc/isohexane) to afford the sub-title compound (EV-3) (395 mg, 1.9 mmol, 28%, 99% Purity) as a yellow solid. m/z 209.1 (M+H)⁺ (ES+). ¹H NMR (400 MHz, DMSO-d6) δ 10.32 (s, 1H), 7.79 (d, J=2.2 Hz, 1H), 7.67 (dd, J=8.4, 2.1 Hz, 1H), 6.87 (d, J=8.4 Hz, 1H), 3.78 (s, 3H), 1.35 (s, 9H).

Step 3: Methyl 3-(tert-butyl)-4-hydroxy-5-nitrobenzoate (EV-4). To a mixture of the product from step 2 above (EV-3) (395 mg, 1 Eq, 1.90 mmol) suspended in conc. H₂SO₄ (6.51 g, 3.56 mL, 35 Eq, 66.4 mmol) was added HNO₃ (299 mg, 198 μL, 2.5 Eq, 4.74 mmol) slowly at 0° C. The resulting suspension was stirred at this temperature for 5 min. The mixture was poured into ice water (10 mL). EtOAc (10 mL) was added, phases separated and aqueous extracted with further EtOAc (2×10 mL). Combined organics were washed with sat. NaHCO₃ (2×10 mL), brine (10 mL), dried (MgSO₄), filtered and evaporated. The crude product was purified by chromatography on silica gel (24 g cartridge, 0-100% EtOAc/isohexane) to afford a mixture of compounds. The mixture was purified by chromatography on silica gel (24 g cartridge, 0-10% EtOAc/isohexane) to afford a mixture of compounds. The crude product was purified by preparative HPLC (Waters, acidic (0.1% formic acid), acidic, Waters X-Select CSH C18 ODB, 5 μm, 30×100 mm column, 40-100% MeCN in water) to afford the title compound (EV-4) (22 mg, 86 μmol, 4.5%, 99% Purity) as white solid. m/z 252.1 (M−H)⁻ (ES−). 1H NMR (400 MHz, DMSO) δ 8.40 (d, J=2.1 Hz, 1H), 8.07 (d, J=2.3 Hz, 1H), 3.86 (s, 3H), 1.42 (s, 9H). OH proton not observed. Further elution also provided 3-(tert-butyl)-4-hydroxy-5-nitrobenzoic acid (24 mg, 99 μmol, 5.2%, 99% Purity) as white solid. m/z 239.1 (M−H)⁻ (ES−). ¹H NMR (400 MHz, DMSO) δ 8.63 (d, J=2.9 Hz, 1H), 8.22 (d, J=2.9 Hz, 1H), 1.42 (s, 9H). Acidic OH not observed.

Step 4: Methyl 3-amino-5-(tert-butyl)-4-hydroxybenzoate (EV-5). To a solution of the product from step 3 above (EV-4) (22 mg, 1 Eq, 87 μmol) in MeOH (1 mL) was added Pd/C 87 L (30 mg, 3.3 Eq, 0.28 mmol). The reaction mixture was stirred at rt with 5 atm of H₂ for 16 h. The catalyst was filtered and the solvent was removed in vacuo to afford the sub-title compound (EV-5) (18 mg, 80 μmol, 92%, 99% Purity) as a brown solid. m/z 224.2 (M+H)⁺ (ES+).

Step 5: Methyl 7-(tert-butyl)-2-(4′-fluoro-2′-(4-methyl-4H-1,2,4-triazol-3-yl)-[1,1′-biphenyl]-3-yl)benzo[d]oxazole-5-carboxylate (EV-6). To a solution of intermediate (B-5) (31 mg, 80% Wt, 1.1 Eq, 89 μmol) in MeOH (3 mL) was added the product from step 4 above (EV-5) (18 mg, 1 Eq, 81 μmol). The resulting mixture was stirred at 45° C. overnight. The reaction mixture was cooled to rt and the volatiles were removed in vacuo. The residue was redissolved in DCM (2 mL). DDQ (20 mg, 1.1 Eq, 89 μmol) was added and the resulting mixture was stirred at rt for 1 h. The reaction mixture was diluted with DCM (10 mL), washed with sat. NaHCO₃ (3×10 mL) and brine. The organic layers were collected, dried (MgSO₄), filtered and concentrated in vacuo. The crude product was purified by chromatography on silica gel (12 g cartridge, 0-10% MeOH/DCM) to afford the sub-title compound (EV-6) (35 mg, 56 μmol, 69%, 77% Purity) as a yellow solid. m/z 485.2 (M+H)⁺ (ES+).

Step 6: (7-(tert-Butyl)-2-(4′-fluoro-2′-(4-methyl-4H-1,2,4-triazol-3-yl)-[1,1′-biphenyl]-3-yl)benzo[d]oxazol-5-yl)methanol (EV-7). To a solution of the product from step 5 above (EV-6) (36 mg, 1 Eq, 74 μmol) in THF (3 mL) was added LiAlH₄ (5.6 mg, 0.15 mL, 1 molar, 2.0 Eq, 0.15 mmol) dropwise at −20° C. This mixture was stirred 1 h at this temperature. The mixture was quenched with H₂O (0.2 mL), and NaOH (aq. 15%, 0.2 mL), and H₂O (0.6 mL) was added at 0° C. The solvent was removed in vacuo. The crude product was purified by chromatography on silica gel (12 g cartridge, 0-10% MeOH/DCM) to the sub-title compound (EV-7) (17 mg, 36 μmol, 49%, 98% Purity) as a colourless solid. m/z 457.2 (M+H)⁺ (ES+). ¹H NMR (400 MHz, MeOD) δ 8.40 (s, 1H), 8.23 (d, J=7.8 Hz, 1H), 8.03 (s, 1H), 7.81 (dd, J=8.7, 5.4 Hz, 1H), 7.65-7.56 (m, 3H), 7.53-7.46 (m, 2H), 7.38 (s, 1H), 4.75 (s, 2H), 3.23 (s, 3H), 1.59 (s, 9H). Exchangeable 1H not observed.

Step 7: 7-(tert-Butyl)-2-(4′-fluoro-2′-(4-methyl-4H-1,2,4-triazol-3-yl)-[1,1′-biphenyl]-3-yl)benzo[d]oxazole-5-carbaldehyde (EV-8). The product from step 6 above (EV-7) (17 mg, 1 Eq, 37 μmol) and Dess-Martin periodinane (32 mg, 2.0 Eq, 74 μmol) were stirred in CHCl₃ (5 mL) at rt for overnight. The reaction mixture was diluted with DCM (10 mL) and washed with sat. aq. NaHCO₃ (5×5 mL) and brine (10 mL). Organics were separated, dried (MgSO₄), filtered and evaporated to afford the sub-title compound (EV-8) (17 mg, 37 μmol, 100%) as a colourless oil. This product was used for next step without further purification.

Step 8: (1S,2R)-2-(((7-(tert-Butyl)-2-(4′-fluoro-2′-(4-methyl-4H-1,2,4-triazol-3-yl)-[1,1′-biphenyl]-3-yl)benzo[d]oxazol-5-yl)methyl)amino)cyclopentan-1-ol (EV-9). A solution of the product from step 7 above (EV-8) (18 mg, 1 Eq, 40 μmol), (1S,2R)-2-aminocyclopentan-1-ol, HCl (R-2) (11 mg, 2 Eq, 79 μmol) and DIPEA (15 mg, 21 μL, 3.0 Eq, 0.12 mmol) in CHCl₃ (1 mL) was stirred for 1 h at rt. NaBH(OAc)₃ (25 mg, 3.0 Eq, 0.12 mmol) was added and the reaction mixture was stirred at rt overnight. The reaction mixture was diluted with EtOAc (10 mL) and washed with sat. aq. sol. of NaHCO₃ (10 mL) and brine (10 mL). The organic layer was extracted, dried (MgSO₄), filtered and concentrated in vacuo. The crude product was purified by chromatography on silica gel (12 g cartridge, 0-30% MeOH/DCM) to afford the title compound (EV-9) (11 mg, 19 μmol, 49%, 95% Purity) as a white solid. m/z 540.3 (M+H)⁺ (ES+). ¹H NMR (400 MHz, DMSO) δ 8.41 (s, 1H), 8.13 (dd, J=7.8, 1.5 Hz, 1H), 7.84 (t, J=1.8 Hz, 1H), 7.77 (dd, J=8.7, 5.7 Hz, 1H), 7.64-7.52 (m, 4H), 7.42-7.37 (m, 1H), 7.29 (d, J=1.6 Hz, 1H), 4.00-3.93 (m, 1H), 3.86 (d, J=13.7 Hz, 1H), 3.80 (d, J=13.5 Hz, 1H), 3.20 (s, 3H), 2.85-2.77 (m, 1H), 1.74-1.57 (m, 4H), 1.49 (s, 9H), 1.45-1.38 (m, 2H). 2× Exchangeable protons not observed.

### Example 220: 3,3-Difluoro-N-((2-(4′-fluoro-2′-(4-methyl-4H-1,2,4-triazol-3-yl)-[1,1′-biphenyl]-3-yl)-7-(trifluoromethyl)benzo[d]oxazol-5-yl)methyl)propan-1-amine (EW-2)

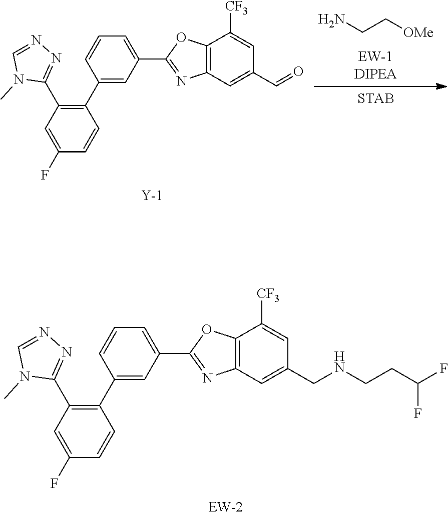

A solution of intermediate (Y-1) (30 mg, 90% Wt, 1 Eq, 58 μmol), DIPEA (22 mg, 30 μL, 3.0 Eq, 0.17 mmol) and 3,3-difluoropropan-1-amine, HCl (EW-1) (15 mg, 2.0 Eq, 0.12 mml) in CHCl₃ (3 mL) was stirred for 1 h at rt. NaBH(OAc)₃ (37 mg, 3.0 Eq, 0.17 mmol) was added and the reaction mixture was stirred at rt overnight. The reaction mixture was diluted with EtOAc (5 mL) and washed with a sat. aq sol. of NaHCO₃ (5 mL) and brine (5 mL). The organic layer was extracted, dried (MgSO₄), and concentrated in vacuo. The crude product was purified by chromatography on silica gel (12 g cartridge, 0-10% MeOH/DCM) to afford the title compound (EW-2) (15.84 mg, 28 μmol, 48%, 95% Purity) as a white solid. m/z 546.2 (M+H)⁺ (ES+). ¹H NMR (400 MHz, DMSO) δ 8.42 (s, 1H), 8.13 (d, J=7.9 Hz, 1H), 8.08 (s, 1H), 7.91 (t, J=1.8 Hz, 1H), 7.79-7.75 (m, 2H), 7.64-7.52 (m, 3H), 7.40-7.36 (m, 1H), 6.33-5.99 (m, 1H), 3.89 (s, 2H), 3.20 (s, 3H), 2.65-2.58 (m, 2H), 2.07-1.92 (m, 2H). One exchangeable proton not observed.

### Example 221: (7-(Difluoromethyl)-2-(4′-fluoro-2′-(4-methyl-4H-1,2,4-triazol-3-yl)-[1,1′-biphenyl]-3-yl)benzo[d]oxazol-5-yl)methanol (EX-6)

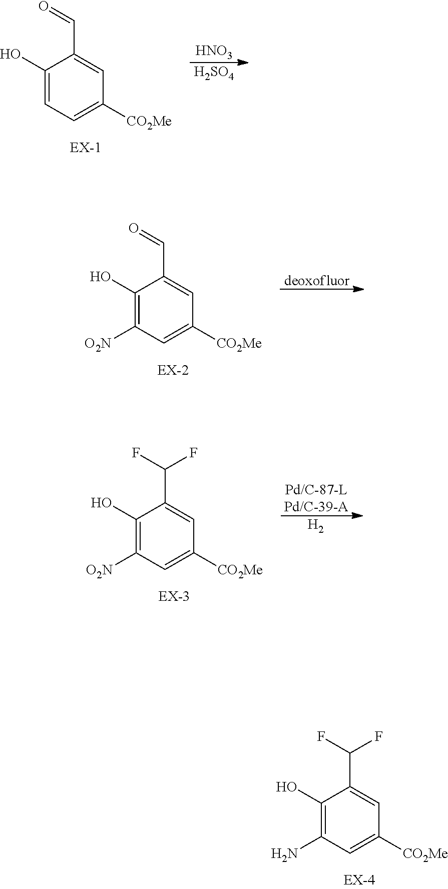

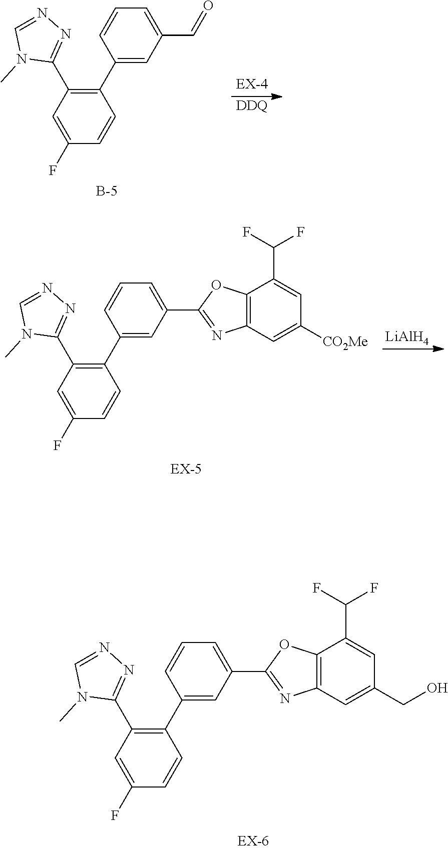

Step 1: Methyl 3-formyl-4-hydroxy-5-nitrobenzoate (EX-2). To a mixture of methyl 3-formyl-4-hydroxybenzoate (EX-1) (1.00 g, 1 Eq, 5.55 mmol) suspended in conc. H₂SO₄ (19.1 g, 10.4 mL, 35 Eq, 194 mmol) was added HNO₃ (874 mg, 579 μL, 2.5 Eq, 13.9 mmol) slowly at 0° C. The resulting suspension was stirred at this temperature for 5 min. The mixture was poured into ice water (10 mL). A precipitation was observed, the product was filtered and purified by chromatography on silica gel (40 g cartridge, 0-50% EtOAc/isohexane) to afford the sub-title compound (EX-2) (1.10 g, 4.8 mmol, 87%, 99% Purity) as an orange solid. m/z 226.1 (M+H)⁺ (ES+). ¹H NMR (400 MHz, DMSO) δ 10.30 (s, 1H), 8.63 (d, J=2.3 Hz, 1H), 8.49 (d, J=2.3 Hz, 1H), 3.89 (s, 3H). OH not observed.

Step 2: Methyl 3-(difluoromethyl)-4-hydroxy-5-nitrobenzoate (EX-3). A solution of the product from step 1 above (EX-2) (500 mg, 1 Eq, 2.22 mmol) in DCM (10 mL) was added to a solution of deoxofluor 50% in THF (983 mg, 819 μL, 50% Wt, 1 Eq, 2.22 mmol). EtOH (20.5 mg, 25.9 μL, 0.2 Eq, 444 μmol) was added to the mixture and the reaction stirred for 16 h. Additional deoxofluor 50% in THF (983 mg, 819 μL, 50% Wt, 1 Eq, 2.22 mmol) was added and the mixture stirred for 16 h. The reaction mixture was quenched with sat. aq. NaHCO₃ (10 mL). DCM (20 mL) was added, and the phases separated. Organics were washed with water (20 mL) and brine (20 mL), dried (MgSO₄), filtered and evaporated. The crude product was purified by chromatography on silica gel (24 g cartridge, 0-100% EtOAc/isohexane) to afford the sub-title compound (EX-3) (138 mg, 0.55 mmol, 25%, 98% Purity) and a second crop of sub-title compound (EX-3) (176 mg, 0.68 mmol, 30%, 95% Purity) as yellow solids. m/z 246.1 (M−H)⁻ (ES−). ¹H NMR (400 MHz, DMSO) δ 8.56 (d, J=2.2 Hz, 1H), 8.23 (s, 1H), 7.21 (t, J=54.4 Hz, 1H), 3.88 (s, 3H). Exchangeable H not observed. ¹⁹F NMR (376 MHz, DMSO) δ −116.15 (d, J=54.4 Hz).

Step 3: Methyl 3-amino-5-(difluoromethyl)-4-hydroxybenzoate (EX-4). To a solution of the product from step 2 above (EX-3) (314 mg, 1 Eq, 1.27 mmol) in MeOH (5 mL) was added Pd/C 87 L (30 mg, 0.22 Eq, 0.28 mmol). The reaction mixture was stirred at 45° C. with 5 atm of H₂ for 16 h. The catalyst was filtered, and additional Pd/C 87 L (30 mg, 0.22 Eq, 0.28 mmol) and MeOH (5 mL) were added. The reaction mixture was stirred at 45° C. with 5 atm of H₂ for 16 h. The catalyst was filtered and Pd/C 39A (36 mg, 0.27 Eq, 0.34 mmol) was added. The reaction mixture was stirred at rt with 4 atm of H₂ for 4 h. The catalyst was filtered, and volatiles evaporated to afford the sub-title compound (EX-4) (147 mg, 0.67 mmol, 53%, 99% Purity) as colourless oil. m/z 218.1 (M+H)⁺ (ES+).

Step 4: Methyl 7-(difluoromethyl)-2-(4′-fluoro-2′-(4-methyl-4H-1,2,4-triazol-3-yl)-[1,1′-biphenyl]-3-yl)benzo[d]oxazole-5-carboxylate (EX-5). To a solution of intermediate (B-5) (100 mg, 1.1 Eq, 356 μmol) in MeOH (3 mL) was added the product from step 3 above (70.2 mg, 1 Eq, 323 μmol). The resulting mixture was stirred at 45° C. overnight. The reaction mixture was cooled to rt and the volatiles were removed in vacuo. The residue was redissolved in DCM (2 mL). DDQ (80.7 mg, 1.1 Eq, 356 μmol) was added and the resulting mixture was stirred at rt for 1 h. The reaction mixture was diluted with DCM (10 mL), washed with sat. NaHCO₃ (3×10 mL) and brine. The organic layers were collected, dried (MgSO₄), filtered and concentrated in vacuo. The crude product was purified by chromatography on silica gel (12 g cartridge, 0-10% MeOH/DCM) to afford the sub-title compound (EX-5) (114 mg, 0.20 mmol, 63%, 85% Purity) as a yellow solid. m/z 479.1 (M+H)⁺ (ES+). ¹H NMR (400 MHz, DMSO) δ 8.51 (s, 1H), 8.42 (d, J=2.6 Hz, 1H), 8.25 (d, J=1.5 Hz, 1H), 8.16 (dd, J=7.9, 1.5 Hz, 1H), 7.97 (t, J=1.8 Hz, 1H), 7.78 (dd, J=8.6, 5.7 Hz, 1H), 7.63-7.54 (m, 4H), 7.44-7.31 (m, 1H), 3.94 (s, 3H), 3.21 (s, 3H).

Step 5: (7-(Difluoromethyl)-2-(4′-fluoro-2′-(4-methyl-4H-1,2,4-triazol-3-yl)-[1,1′-biphenyl]-3-yl)benzo[d]oxazol-5-yl)methanol (EX-6). To a solution of the product from step 4 above (EX-5) (114 mg, 1 Eq, 238 μmol) in THF (5 mL) was added LiAlH₄ (18.1 mg, 477 μL, 1 molar, 2.0 Eq, 477 μmol) dropwise at −20° C. This mixture was stirred 1 h at this temperature. The mixture was quenched with H₂O (0.2 mL), and NaOH (aq. 15%, 0.2 mL), and H₂O (0.6 mL) was added at 0° C. The solvent was removed in vacuo. The crude product was purified by chromatography on silica gel (12 g cartridge, 0-10% MeOH/DCM) to afford impure product. The crude product was purified by preparative HPLC (Waters, basic (0.1% ammonia), basic, Waters XBridge BEH C18 ODB, 5 μm, 30×100 mm column, 20-100% MeCN in water) to afford the title compound (EX-6) (25 mg, 55 μmol, 23%, 99% Purity). m/z 451.1 (M+H)⁺ (ES+). ¹H NMR (400 MHz, DMSO) δ 8.42 (s, 1H), 8.14 (d, J=7.9 Hz, 1H), 7.95 (t, J=1.9 Hz, 1H), 7.89 (s, 1H), 7.78 (dd, J=8.6, 5.6 Hz, 1H), 7.61 (q, J=3.3 Hz, 2H), 7.60-7.46 (m, 3H), 7.36-7.31 (m, 1H), 5.47 (s, 1H), 4.67 (s, 2H), 3.20 (s, 3H).

### Example 222: (1S,2R)-2-(((7-(Difluoromethyl)-2-(4′-fluoro-2′-(4-methyl-4H-1,2,4-triazol-3-yl)-[1,1′-biphenyl]-3-yl)benzo[d]oxazol-5-yl)methyl)amino)cyclopentan-1-ol (EY-2)

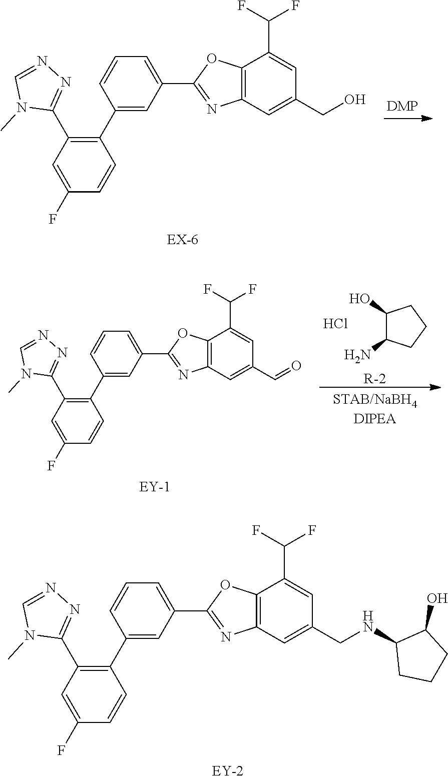

Step 1: 7-(Difluoromethyl)-2-(4′-fluoro-2′-(4-methyl-4H-1,2,4-triazol-3-yl)-[1,1′-biphenyl]-3-yl)benzo[d]oxazole-5-carbaldehyde (EY-1). Compound (EX-6) (37 mg, 1 Eq, 82 μmol) and Dess-Martin periodinane (70 mg, 2.0 Eq, 0.16 mmol) were stirred in DCM (5 mL) at rt overnight. The reaction was heated to 45° C. and stirred for another 16 h. The reaction mixture was diluted with DCM (10 mL) and washed with sat. aq. NaHCO₃ (5×5 mL) and brine (10 mL). Organics were separated, dried (MgSO₄), filtered and evaporated to afford the sub-title compound (37 mg, 82 μmol, 100%) as a colourless oil. This product was used for next step without further purification or analysis.

Step 2: (1S,2R)-2-(((7-(Difluoromethyl)-2-(4′-fluoro-2′-(4-methyl-4H-1,2,4-triazol-3-yl)-[1,1′-biphenyl]-3-yl)benzo[d]oxazol-5-yl)methyl)amino)cyclopentan-1-ol (EY-2). A solution of the product from step 1 above (EY-1) (37 mg, 1 Eq, 83 μmol), (1S,2R)-2-aminocyclopentan-1-ol, HCl (R-2) (23 mg, 2 Eq, 0.17 mmol) and DIPEA (32 mg, 43 μL, 3.0 Eq, 0.25 mmol) in CHCl₃ (1 mL) was stirred for 1 h at rt. NaBH(OAc)₃ (52 mg, 3.0 Eq, 0.25 mmol) was added and the reaction mixture was stirred at rt overnight. The reaction mixture was diluted with EtOAc (10 mL) and washed with sat. aq. sol. of NaHCO₃ (10 mL) and brine (10 mL). The organic layer was extracted, dried (MgSO₄), filtered and concentrated in vacuo. The crude product was purified by chromatography on silica gel (12 g cartridge, 0-30% MeOH/DCM) to afford the title compound (EY-2) (14 mg, 25 μmol, 30%, 95% Purity) as a white solid. m/z 534.2 (M+H)⁺ (ES+). ¹H NMR (400 MHz, DMSO) δ 8.42 (s, 1H), 8.13 (d, J=7.8 Hz, 1H), 7.97-7.92 (m, 2H), 7.78 (dd, J=8.6, 5.6 Hz, 1H), 7.66 (s, 1H), 7.63-7.32 (m, 5H), 4.04-3.85 (m, 3H), 3.20 (s, 3H), 2.87-2.78 (m, 1H), 1.79-1.55 (m, 6H). 2 Exchangeable protons not observed.

### Example 223: N-((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)-2-methoxyethan-1-amine (EZ-3)

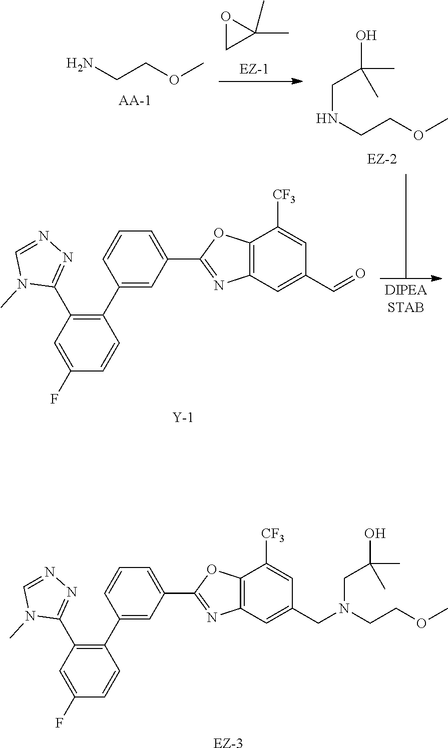

Step 1: 1-((2-Methoxyethyl)amino)-2-methylpropan-2-ol (EZ-2). 2,2-Dimethyloxirane (EZ-1) (0.8 g, 1 mL, 2 Eq, 0.01 mol) and 2-methoxyethan-1-amine (AA-1) (0.4 g, 0.5 mL, 1 Eq, 5 mmol) were added to a microwave vial and sealed. The reaction mixture was heated to 50° C. for 5 days to afford crude sub-title compound (EZ-2) (824.6 mg, 5.0 mmol, 90%, 90% Purity) which was used crude without further purification. ¹H NMR (400 MHz, DMSO) δ 3.38 (t, J=5.6 Hz, 2H), 3.24 (s, 3H), 2.68-2.62 (m, 2H), 2.39 (s, 2H), 1.07 (s, 6H). Two exchangeable protons not observed.

Step 2: N-((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)-2-methoxyethan-1-amine (EZ-3). A solution of intermediate (Y-1) (40 mg, 90% Wt, 1 Eq, 77 μmol), DIPEA (30 mg, 40 μL, 3.0 Eq, 0.23 mmol) and the product from step 1 above (EZ-2) (25 mg, 90% Wt, 2.0 Eq, 0.15 mmol) in CHCl₃ (3 mL) was stirred for 16 h at rt. The reaction mixture was heated to 50° C. overnight. NaBH(OAc)₃ (49 mg, 3.0 Eq, 0.23 mmol) was added to the reaction mixture and stirred for 16 h at 50° C. NaBH₄ (8.8 mg, 3 Eq, 0.23 mmol) was added and the reaction was stirred for 16 h at 50° C. The reaction mixture was diluted with EtOAc (5 mL) and washed with a sat. aq sol. of NaHCO₃ (5 mL) and brine (5 mL). The organic layer was extracted, dried (MgSO₄), and concentrated in vacuo. The crude product was purified by preparative HPLC (Waters, Basic (0.1% ammonium bicarbonate), Basic, Waters X-Bridge Prep-C18, 5 μm, 30×100 mm column, 0-100% MeCN in water) to afford the title compound (EZ-3) (1.70 mg, 77 μmol, 3.6%, 98% Purity) as a white solid. m/z 598.0 (M+H)⁺ (ES+). ¹H NMR (400 MHz, MeOD) δ 8.41 (s, 1H), 8.26-8.21 (m, 1H), 8.10-8.07 (m, 1H), 8.06 (s, 1H), 7.85 (s, 1H), 7.84-7.78 (m, 1H), 7.63-7.55 (m, 2H), 7.51-7.45 (m, 2H), 4.02 (s, 2H), 3.52 (t, J=5.5 Hz, 2H), 3.35 (s, 3H), 3.27 (s, 3H), 2.81 (t, J=5.5 Hz, 2H), 2.64 (s, 2H), 1.20 (s, 6H). One exchangeable proton not observed.

### Example 224: 1-Cyclobutyl-N-((2-(4′-fluoro-2′-(4-methyl-4H-1,2,4-triazol-3-yl)-[1,1′-biphenyl]-3-yl)-7-methoxybenzo[d]oxazol-5-yl)methyl)methanamine (FA-1)

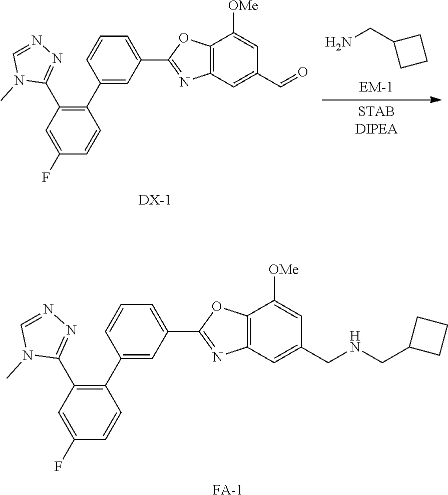

A solution of Intermediate (DX-1) (27 mg, 1 Eq, 63 μmol), cyclobutylmethanamine (EM-1) (11 mg, 0.01 mL, 2.0 Eq, 0.13 mmol) and DIPEA (24 mg, 33 μL, 3.0 Eq, 0.19 mmol) in DCM (2 mL) was stirred for 1 h at rt. NaBH(OAc)₃ (40 mg, 3.0 Eq, 0.19 mmol) was added and the reaction mixture was stirred at rt overnight. The reaction mixture was diluted with EtOAc (10 mL) and washed with sat. aq. sol. of NaHCO₃ (10 mL). The organic layer was extracted, dried (MgSO₄), filtered and concentrated in vacuo. The crude product was purified by chromatography on silica gel (12 g cartridge, 0-10% MeOH/DCM) to afford the title compound (FA-1) (15 mg, 29 μmol, 45%, 95% Purity) as a white solid. m/z 498.2 (M+H)⁺ (ES+). ¹H NMR (400 MHz, DMSO) δ 8.41 (s, 1H), 8.13-8.09 (m, 1H), 7.96 (d, J=1.8 Hz, 1H), 7.78 (dd, J=8.6, 5.7 Hz, 1H), 7.60 (td, J=8.6, 2.8 Hz, 1H), 7.55-7.49 (m, 2H), 7.32 (s, 1H), 7.26 (d, J=8.0 Hz, 1H), 7.09-7.06 (m, 1H), 4.00 (s, 3H), 3.79 (s, 2H), 3.13 (s, 3H), 2.03-1.95 (m, 2H), 1.87-1.58 (m, 5H). 3H under DMSO peak.

### Example 225: 1-Cyclopropyl-N-((2-(4′-fluoro-2′-(4-methyl-4H-1,2,4-triazol-3-yl)-[1,1′-biphenyl]-3-yl)-7-(trifluoromethyl)benzo[d]oxazol-5-yl)methyl)methanamine (FB-2)

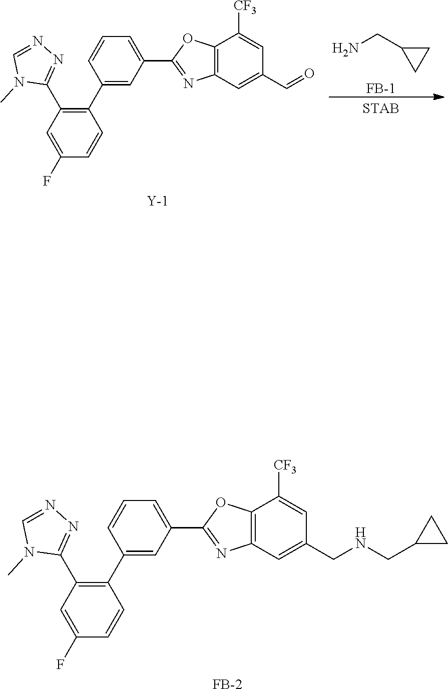

A solution of intermediate (Y-1) (30 mg, 90% Wt, 1 Eq, 58 μmol) and cyclopropylmethanamine (FB-1) (8.2 mg, 2 Eq, 0.12 mmol) in CHCl₃ (3 mL) was stirred for 1 h at rt. NaBH(OAc)₃ (37 mg, 3.0 Eq, 0.17 mmol) was added and the reaction mixture was stirred at rt overnight. The reaction mixture was diluted with EtOAc (5 mL) and washed with a sat. aq sol. of NaHCO₃ (5 mL) and brine (5 mL). The organic layer was extracted, dried (MgSO₄), and concentrated in vacuo. The crude product was purified by chromatography on silica gel (4 g cartridge, 0-10% MeOH/DCM) to afford the title compound (FB-2) (13.95 mg, 26 μmol, 44%, 96% Purity) as a white solid. m/z 522.1 (M+H)⁺ (ES+). ¹H NMR (400 MHz, DMSO) δ 8.42 (s, 1H), 8.13 (d, J=8.1 Hz, 1H), 8.06 (s, 1H), 7.91 (d, J=2.0 Hz, 1H), 7.80-7.75 (m, 2H), 7.64-7.52 (m, 3H), 7.37 (d, J=7.9 Hz, 1H), 3.90 (s, 2H), 3.20 (s, 3H), 2.38 (d, J=6.6 Hz, 2H), 0.96-0.85 (m, 1H), 0.43-0.36 (m, 2H), 0.12-0.05 (m, 2H). One exchangeable proton not observed.

### Example 226: 1-(2-(4′-Fluoro-2′-(4-methyl-4H-1,2,4-triazol-3-yl)-[1,1′-biphenyl]-3-yl)-7-(trifluoromethyl)benzo[d]oxazol-5-yl)-N-((1-(methoxymethyl)cyclopentyl)methyl)methanamine (FC-2)

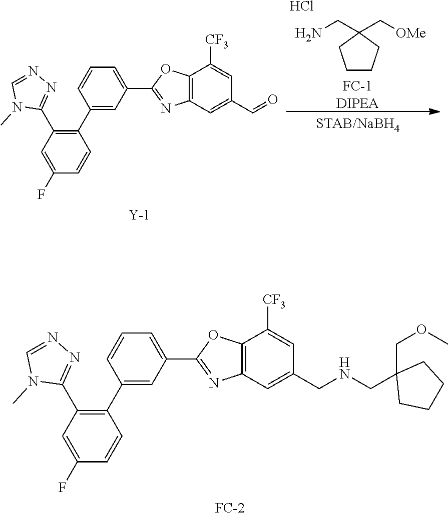

NaBH(OAc)₃ (37 mg, 3.0 Eq, 0.17 mmol) was added to a stirred mixture of intermediate (Y-1) (30 mg, 90% Wt, 1 Eq, 58 μmol), [1-(methoxymethyl)cyclopentyl]methanamine, HCl (FC-1) (21 mg, 2.0 Eq, 0.12 mmol) and DIPEA (22 mg, 30 μL, 3.0 Eq, 0.17 mmol) in CHCl₃ (3 mL). The reaction mixture was stirred at rt overnight. NaBH₄ (11 mg, 5.0 Eq, 0.29 mmol) was added to the reaction mixture and stirring continued at rt overnight. The reaction mixture was diluted with DCM (4 mL) and washed with sat. aq. NaHCO₃ (10 mL). The organic phase was separated and evaporated to afford the crude product. The crude product was purified by preparative HPLC (Waters, Basic (0.10% ammonia), Basic, Waters X-Bridge BEH C18 ODB, 5 μm, 30×100 mm column, 50-100% MeCN in water) to afford the title compound (FC-2) (19 mg, 31 μmol, 54%, 97% Purity) as a colourless foam. m/z 594.6 (M+H)⁺ (ES+). ¹H NMR (400 MHz, DMSO) δ 8.41 (s, 1H), 8.13 (dt, J=7.9, 1.4 Hz, 1H), 8.05 (s, 1H), 7.90 (t, J=1.8 Hz, 1H), 7.82-7.73 (m, 2H), 7.65-7.51 (m, 3H), 7.38 (dt, J=7.7, 1.5 Hz, 1H), 3.91-3.86 (m, 2H), 3.23 (s, 3H), 3.20 (s, 3H), 3.18 (s, 2H), 2.40-2.34 (m, 2H), 2.28-2.23 (m, 1H), 1.54-1.45 (m, 4H), 1.43-1.31 (m, 4H).

### Example 227: 2-fluoro-N-((2-(4′-fluoro-2′-(4-methyl-4H-1,2,4-triazol-3-yl)-[1,1′-biphenyl]-3-yl)-7-(trifluoromethyl)benzo[d]oxazol-5-yl)methyl)ethan-1-amine (FD-2)

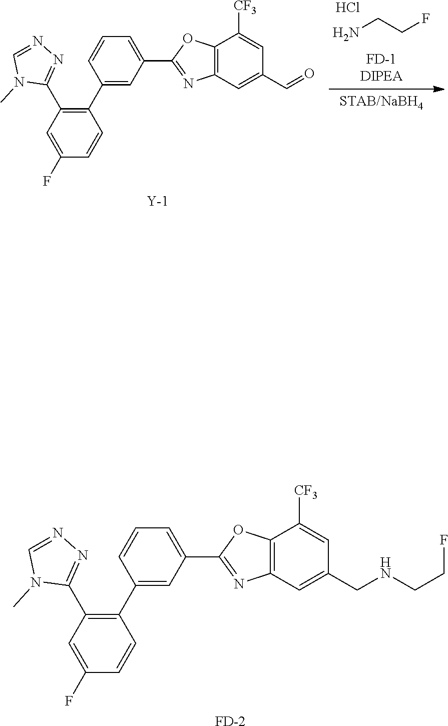

NaBH(OAc)₃ (37 mg, 3.0 Eq, 0.17 mmol) was added to a stirred mixture of intermediate (Y-1) (30 mg, 90% Wt, 1 Eq, 58 μmol), 2-fluoroethanamine, HCl (FD-1) (12 mg, 2 Eq, 0.12 mmol) and DIPEA (22 mg, 30 μL, 3.0 Eq, 0.17 mmol) in CHCl₃ (3 mL). The reaction mixture was stirred at rt overnight. NaBH₄ (11 mg, 5.0 Eq, 0.29 mmol) was added to the reaction mixture and stirring continued at rt overnight. The reaction mixture was diluted with DCM (4 mL) and washed with sat. aq. NaHCO₃ (10 mL). The organic phase was separated and evaporated to afford the crude product. The crude product was purified by preparative HPLC (Waters, Basic (0.1% ammonia), Basic, Waters X-Bridge BEH C18 ODB, 5 μm, 30×100 mm column, 30-100% MeCN in water) to afford the title compound (FD-2) (17 mg, 32 μmol, 55%, 96% Purity) as a colourless foam. m/z 514.2 (M+H)⁺ (ES+). ¹H NMR (400 MHz, DMSO) δ 8.42 (s, 1H), 8.13 (dt, J=7.9, 1.3 Hz, 1H), 8.08 (s, 1H), 7.91 (t, J=1.8 Hz, 1H), 7.81-7.73 (m, 2H), 7.65-7.51 (m, 3H), 7.38 (dt, J=7.9, 1.4 Hz, 1H), 4.55 (t, J=5.0 Hz, 1H), 4.43 (t, J=5.0 Hz, 1H), 3.93 (s, 2H), 3.20 (s, 3H), 2.82 (t, J=5.0 Hz, 1H), 2.75 (t, J=5.1 Hz, 1H), 2.61 (s, 1H).

### Example 228: (2-(4′-Fluoro-2′-(4-methyl-4H-1,2,4-triazol-3-yl)-[1,1′-biphenyl]-3-yl)-7-methylbenzo[d]oxazol-5-yl)methanol (FE-3)

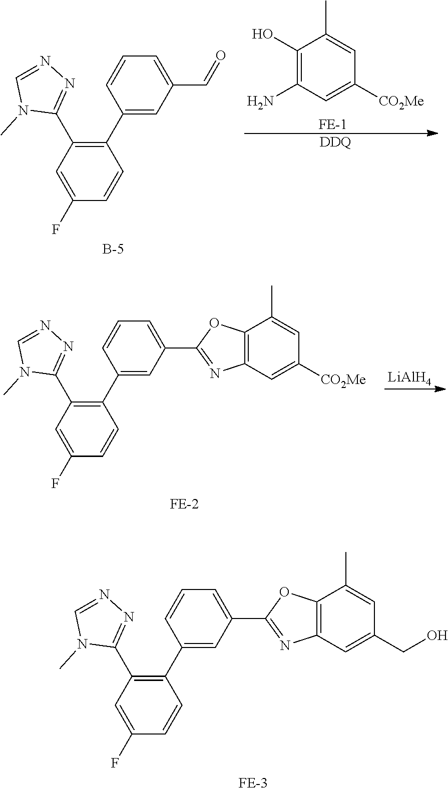

Step 1: Methyl 2-(4′-fluoro-2′-(4-methyl-4H-1,2,4-triazol-3-yl)-[1,1′-biphenyl]-3-yl)-7-methylbenzo[d]oxazole-5-carboxylate (FE-2). A solution of intermediate (B-5) (208.9 mg, 77% Wt, 1.036 Eq, 571.8 μmol) and methyl 3-amino-4-hydroxy-5-methylbenzoate (FE-1) (100.0 mg, 1 Eq, 551.9 μmol) in MeOH (5.0 mL) was heated at 45° C. overnight. The reaction mixture was cooled to rt and the volatiles were removed in vacuo. The residue was redissolved in DCM (5.0 mL). DDQ (132.1 mg, 98% Wt, 1.033 Eq, 570.3 μmol) was added and the resulting mixture was stirred at rt for 1 h. The reaction mixture was diluted with DCM (10 mL), washed with sat. NaHCO₃ (3×10 mL) and brine (10 mL). The organic layers were collected, dried (Na₂SO₄), filtered and concentrated in vacuo. The crude product was purified by chromatography on silica gel (12 g cartridge, 0-10% MeOH/DCM) to afford the sub-title compound (FE-2) (242.4 mg, 0.41 mmol, 100%, 74% Purity) as a light brown solid. m/z 443.3 (M+H)⁺ (ES+). ¹H NMR (400 MHz, DMSO) δ 8.44 (s, 1H), 8.19-8.11 (m, 2H), 7.96 (t, J=1.8 Hz, 1H), 7.92 (s, 1H), 7.79 (dd, J=8.6, 5.7 Hz, 1H), 7.64-7.48 (m, 3H), 7.37 (d, J=7.8 Hz, 1H), 3.90 (s, 3H), 3.16 (s, 3H), 2.62 (s, 3H).

Step 2: (2-(4′-Fluoro-2′-(4-methyl-4H-1,2,4-triazol-3-yl)-[1,1′-biphenyl]-3-yl)-7-methylbenzo[d]oxazol-5-yl)methanol (FE-3). To a solution of the product from step 1 above (242.4 mg, 74% Wt, 1 Eq, 405.4 μmol) in THF (3.0 mL) was added LiAlH₄ in THF (15.39 mg, 405.4 μL, 1 molar, 1 Eq, 405.4 μmol) dropwise at 0° C. This mixture was stirred at 0° C. for 1 h. The mixture was quenched with NaOH (2 M, 0.5 mL) at 0° C. MgSO₄ was added. The mixture was warmed up to rt, stirred for 15 min, filtered and the solvent was removed in vacuo. The crude product was purified by chromatography on silica gel (12 g cartridge, 0-10% MeOH/DCM) to afford impure product. The crude product was purified by preparative HPLC (Waters, acidic (0.1% formic acid), acidic, Waters X-Select CSH C18 ODB, 5 μm, 30×100 mm column, 20-100% MeCN in water) to afford the title compound (FE-3) (92.0 mg, 0.22 mmol, 54%, 99% Purity) as a white solid. m/z 415.2 (M+H)⁺ (ES+). ¹H NMR (400 MHz, DMSO) δ 8.43 (s, 1H), 8.16-8.11 (m, 1H), 7.95-7.92 (m, 1H), 7.79 (dd, J=8.6, 5.6 Hz, 1H), 7.60 (td, J=8.6, 2.8 Hz, 1H), 7.56-7.51 (m, 3H), 7.34-7.30 (m, 1H), 7.23-7.19 (m, 1H), 5.27 (t, J=5.8 Hz, 1H), 4.58 (d, J=5.7 Hz, 2H), 3.16 (s, 3H), 2.55 (s, 3H).

### Example 229: (7-(1,1-Difluoroethyl)-2-(4′-fluoro-2′-(4-methyl-4H-1,2,4-triazol-3-yl)-[1,1′-biphenyl]-3-yl)benzo[d]oxazol-5-yl)methanol (FF-6)

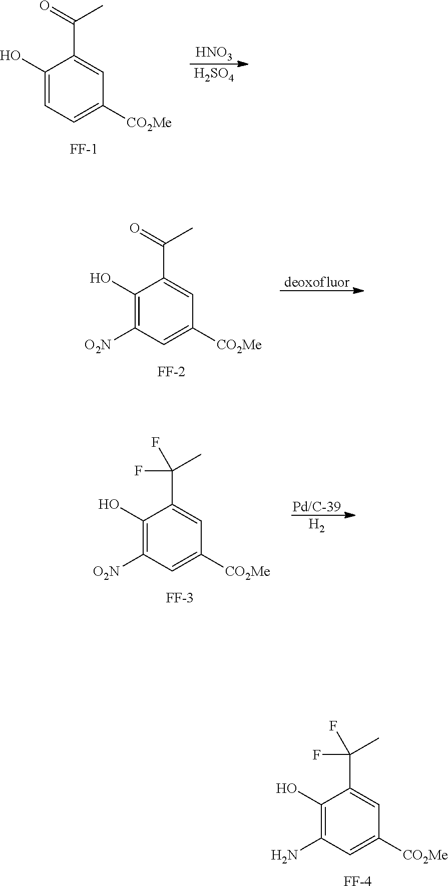

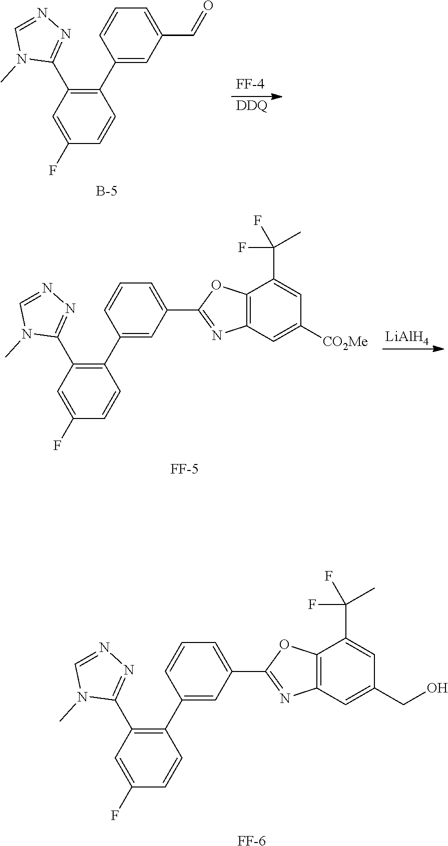

Step 1: Methyl 3-acetyl-4-hydroxy-5-nitrobenzoate (FF-2). To a mixture of methyl 3-acetyl-4-hydroxybenzoate (FF-1) (500 mg, 1 Eq, 2.57 mmol) suspended in conc. H₂SO₄ (8.84 g, 4.83 mL, 35 Eq, 90.1 mmol) was added HNO₃ (406 mg, 269 μL, 2.5 Eq, 6.44 mmol) slowly at 0° C. The resulting suspension was stirred at this temperature for 5 min. The mixture was poured into ice water (10 mL). EtOAc (10 mL) was added, phases separated and aqueous extracted with further EtOAc (2×10 mL). Combined organics were washed with sat. NaHCO₃ (2×10 mL). The aqueous layers were acidified and the initial acidic aqueous wash and extracted with EtOAc (3×10 mL). The combined organic layers were washed with brine (10 mL), dried (Na₂SO₄), filtered and the volatiles were removed in vacuo to afford the sub-title compound (FF-2) (1.0802 g, 2.6 mmol, 100%, 58% Purity) as a dark brown solid. m/z 240.1 (M+H)⁺ (ES+). ¹H NMR (400 MHz, DMSO) δ 8.46 (d, J=2.1 Hz, 1H), 8.41 (d, J=2.4 Hz, 1H), 3.83 (s, 3H), 2.66 (s, 3H). 1× exchangeable OH proton not observed.

Step 2: Methyl 3-(1,1-difluoroethyl)-4-hydroxy-5-nitrobenzoate (FF-3). To a stirred solution of the product from step 1 above (FF-2) (1.0802 g, 58% Wt, 1 Eq, 2.6194 mmol) in dry DCM (10 mL) under a nitrogen atmosphere at rt was added deoxofluor 50% in THF (1.2 g, 1.0 mL, 50% Wt, 1.0 Eq, 2.7 mmol) and EtOH (24 mg, 30 μL, 0.20 Eq, 0.51 mmol). The reaction mixture was stirred at rt overnight. Additional deoxofluor 50% in THF (1.2 g, 1.0 mL, 50% Wt, 1.0 Eq, 2.7 mmol) was added and the mixture stirred at rt overnight. The reaction was heated at 40° C. for 2 h and then cooled to rt. Additional deoxofluor 50% in THF (1.1590 g, 2.0 mL, 50% Wt, 1 Eq, 2.6194 mmol) and EtOH (47 mg, 60 μL, 0.39 Eq, 1.0 mmol) was added and the resulting mixture was heated at 40° C. overnight. The reaction mixture was cooled to rt, diluted with DCM (10 mL) and quenched with sat. NaHCO₃ (10 mL). The organic layer was washed with distilled water (10 mL) and brine (10 mL), dried (Na₂SO₄), filtered and concentrated in vacuo. The crude product was purified by chromatography on silica gel (24 g cartridge, 0-100% EtOAc/isohexane) to afford the sub-title compound (FF-3) (82.9 mg, 0.29 mmol, 11%, 90% Purity) as a yellow oil. m/z 262.0 (M+H)⁺ (ES+); 260.0 (M−H)⁻ (ES−). ¹H NMR (400 MHz, DMSO) δ 8.54 (d, J=2.3 Hz, 1H), 8.19 (d, J=2.2 Hz, 1H), 3.87 (s, 3H), 2.05 (t, J=19.3 Hz, 3H). 1× exchangeable OH proton not observed. ¹⁹F NMR (376 MHz, DMSO) δ −85.79 (q, J=19.3 Hz).

Step 3: Methyl 3-amino-5-(1,1-difluoroethyl)-4-hydroxybenzoate (FF-4). To a solution of the product from step 2 above (FF-3) (82.9 mg, 90% Wt, 1 Eq, 286 μmol) in MeOH (5 mL) was added Pd/C Type 39 (31.5 mg, 10% Wt, 0.104 Eq, 29.6 μmol). The reaction mixture was stirred at rt with 5 atm of H₂ overnight. The mixture was filtered, and the volatiles were removed in vacuo to afford the sub-title compound (FF-4) (73.8 mg, 0.19 mmol, 68%, 61% Purity) as a brown oil, which was used directly without further purification. m/z 232.1 (M+H)⁺ (ES+). ¹H NMR (400 MHz, DMSO) δ 7.40 (d, J=2.2 Hz, 1H), 7.29 (d, J=2.1 Hz, 1H), 3.78 (s, 3H), 1.98 (t, J=19.0 Hz, 3H). 3× exchangeable protons not observed. ¹⁹F NMR (376 MHz, DMSO) δ −84.99 (q, J=19.0 Hz).

Step 4: Methyl 7-(1,1-difluoroethyl)-2-(4′-fluoro-2′-(4-methyl-4H-1,2,4-triazol-3-yl)-[1,1′-biphenyl]-3-yl)benzo[d]oxazole-5-carboxylate (FF-5). A solution of intermediate (B-5) (75.5 mg, 77% Wt, 1.06 Eq, 207 μmol) and the product from step 3 above (FF-4) (73.8 mg, 61% Wt, 1 Eq, 195 μmol) in MeOH (5.0 mL) was heated at 45° C. overnight. The reaction mixture was cooled to rt and the volatiles were removed in vacuo. The residue was redissolved in DCM (5.0 mL). DDQ (53.1 mg, 98% Wt, 1.18 Eq, 229 μmol) was added and the resulting mixture was stirred at rt for 2 h. The reaction mixture was diluted with DCM (10 mL), washed with sat. NaHCO₃ (3×10 mL) and brine (10 mL). The organic layers were collected, dried (Na₂SO₄), filtered and concentrated in vacuo. The crude product was purified by chromatography on silica gel (12 g cartridge, 0-10% MeOH/DCM) to afford the sub-title compound (FF-5) (33.0 mg, 59 μmol, 15%, 88% Purity) as a dark brown solid. m/z 493.1 (M+H)⁺ (ES+). ¹H NMR (400 MHz, DMSO) δ 8.49-8.46 (m, 1H), 8.42 (s, 1H), 8.20-8.17 (m, 1H), 8.14-8.11 (m, 1H), 7.94-7.91 (m, 1H), 7.78 (dd, J=8.5, 5.7 Hz, 1H), 7.65-7.53 (m, 3H), 7.45-7.41 (m, 1H), 3.93 (s, 3H), 3.20 (s, 3H), 2.25 (t, J=19.3 Hz, 3H). ¹⁹F NMR (376 MHz, DMSO) δ −84.49 (q, J=19.2 Hz), −113.56-−113.65 (m).

Step 5: (7-(1,1-Difluoroethyl)-2-(4′-fluoro-2′-(4-methyl-4H-1,2,4-triazol-3-yl)-[1,1′-biphenyl]-3-yl)benzo[d]oxazol-5-yl)methanol (FF-6). To a solution of the product from step 4 above (FF-5) (33.0 mg, 88% Wt, 1 Eq, 59.0 μmol) in THF (2.0 mL) was added LiAlH₄ in THF (2.24 mg, 59.0 μL, 1 molar, 1 Eq, 59.0 μmol) dropwise at 0° C. This mixture was stirred at 0° C. for 1 h. The mixture was quenched with NaOH (2 M, 0.5 mL) at 0° C. Sodium sulfate was added. The mixture was warmed up to rt, stirred for 15 min, filtered and the volatiles were removed in vacuo. The crude product was purified by chromatography on silica gel (4 g cartridge, 0-10% MeOH/DCM) to afford the title compound FF-6) (19.9 mg, 42 μmol, 70%, 97% Purity) as a pale-yellow solid. m/z 465.2 (M+H)⁺ (ES+). ¹H NMR (400 MHz, MeOD) δ 8.38 (s, 1H), 8.23-8.18 (m, 1H), 8.03-8.00 (m, 1H), 7.84-7.82 (m, 1H), 7.79 (dd, J=8.5, 5.4 Hz, 1H), 7.62-7.51 (m, 3H), 7.49-7.44 (m, 2H), 4.76 (s, 2H), 3.23 (s, 3H), 2.19 (t, J=18.6 Hz, 3H). 1× exchangeable OH proton not observed. ¹⁹F NMR (376 MHz, MeOD) δ −87.43 (q, J=18.7 Hz), −114.99-−115.07 (m).

### Example 230: (1S,2R)-2-(((2-(4′-Fluoro-2′-(4-methyl-4H-1,2,4-triazol-3-yl)-[1,1′-biphenyl]-3-yl)-7-methylbenzo[d]oxazol-5-yl)methyl)amino)cyclopentan-1-ol (FG-2)

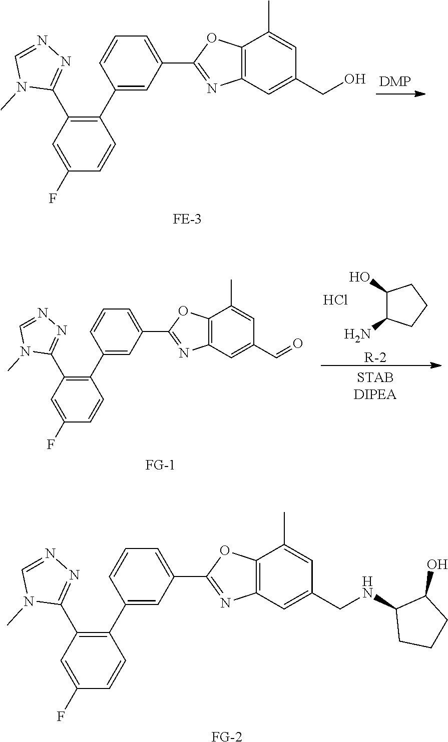

Step 1: 2-(4′-Fluoro-2′-(4-methyl-4H-1,2,4-triazol-3-yl)-[1,1′-biphenyl]-3-yl)-7-methylbenzo[d]oxazole-5-carbaldehyde (FG-1). To a stirred solution of intermediate (FE-3) (89.8 mg, 99% Wt, 1 Eq, 215 μmol) in dry DCM (3.0 mL) at rt was added Dess-Martin periodinane (140.5 mg, 1.54 Eq, 331.3 μmol). The reaction mixture was stirred at rt for 1 h. Additional Dess-Martin periodinane (143.5 mg, 1.58 Eq, 338.3 μmol) was added and the resulting mixture was stirred at rt for 1 h. A further portion of Dess-Martin periodinane (143.8 mg, 1.58 Eq, 339.0 μmol) was added and the reaction mixture was stirred at rt for 3 d. The reaction mixture was diluted with DCM (5 mL), washed with sat. NaHCO₃ (5 mL) and brine (5 mL). The organic layer was dried (Na₂SO₄), filtered and concentrated in vacuo. The crude product was purified by chromatography on silica gel (12 g cartridge, 0-10% MeOH/DCM) to afford the sub-title compound (FG-1) (95.5 mg, 0.20 mmol, 92%, 85% Purity) as a white solid. m/z 413.1 (M+H)⁺ (ES+). ¹H NMR (400 MHz, DMSO) δ 10.08 (s, 1H), 8.44 (s, 1H), 8.21-8.15 (m, 2H), 7.97 (t, J=1.9 Hz, 1H), 7.87-7.83 (m, 1H), 7.80 (dd, J=8.7, 5.7 Hz, 1H), 7.65-7.51 (m, 3H), 7.41-7.35 (m, 1H), 3.17 (s, 3H), 2.64 (s, 3H).

Step 2: (1S,2R)-2-(((2-(4′-Fluoro-2′-(4-methyl-4H-1,2,4-triazol-3-yl)-[1,1′-biphenyl]-3-yl)-7-methylbenzo[d]oxazol-5-yl)methyl)amino)cyclopentan-1-ol (FG-2). A solution of the product from step 1 above (FG-1) (28.1 mg, 85% Wt, 1 Eq, 57.9 μmol), (1S,2R)-2-aminocyclopentan-1-ol, HCl (R-2) (17.7 mg, 2.22 Eq, 129 μmol) and DIPEA (45 mg, 60 μL, 5.9 Eq, 0.34 mmol) in CHCl₃ (3.0 mL) was stirred 1 h at rt. NaBH(OAc)₃ (42.1 mg, 97% Wt, 3.33 Eq, 193 μmol) was added and the reaction mixture was stirred at rt overnight. Additional NaBH(OAc)₃ (43.1 mg, 97% Wt, 3.41 Eq, 197 μmol) was added and the resulting mixture was stirred at rt for 1 h. The reaction mixture was diluted with EtOAc (2 mL), washed with sat. NaHCO₃ (2 mL) and brine (2 mL), dried (Na₂SO₄), filtered and concentrated in vacuo. The crude product was purified by chromatography on silica gel (4 g cartridge, 0-20% MeOH/DCM) to afford the title compound (FG-2) (21.1 mg, 41 μmol, 71%, 97% Purity) as a white solid. m/z 498.2 (M+H)⁺ (ES+). ¹H NMR (400 MHz, DMSO) δ 8.43 (s, 1H), 8.15-8.11 (m, 1H), 7.92 (t, J=1.8 Hz, 1H), 7.79 (dd, J=8.6, 5.7 Hz, 1H), 7.63-7.51 (m, 4H), 7.36-7.31 (m, 1H), 7.26 (s, 1H), 4.01-3.96 (m, 1H), 3.90-3.78 (m, 2H), 3.16 (s, 3H), 2.92-2.79 (m, 1H), 2.55 (s, 3H), 1.80-1.53 (m, 4H), 1.49-1.36 (m, 2H). 2× Exchangeable protons not observed.

### Example 231: 2-(4′-Fluoro-2′-(4-methyl-4H-1,2,4-triazol-3-yl)-[1,1′-biphenyl]-3-yl)-4-methoxybenzo[d]oxazole (FH-2)

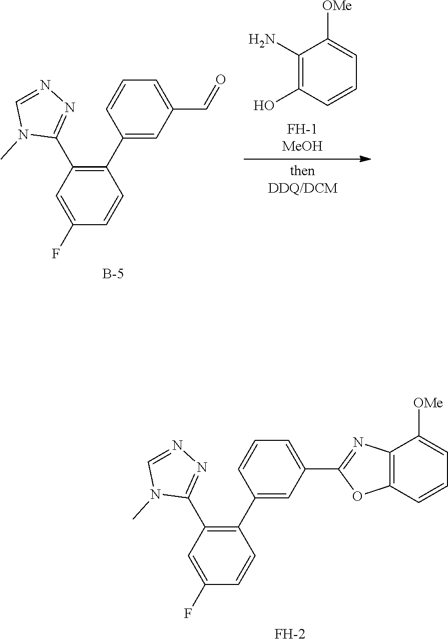

To a solution of intermediate (B-5) (30 mg, 1 Eq, 0.11 mmol) in MeOH (1 mL) was added 2-amino-6-methoxyphenol (FH-1) (15 mg, 1 Eq, 0.11 mmol). The resulting mixture was stirred at 45° C. for 12 h. After concentration in vacuo, the residue was dissolved in DCM (1 mL) and DDQ (27 mg, 1.1 Eq, 0.12 mmol) was then added. The mixture was stirred at rt for 1 h. A sat. solution of NaHCO₃ (5 mL) was added and extracted with DCM (2×5 mL). Organics were combined, dried (MgSO₄), filtered and evaporated. The crude was purified by preparative HPLC (Waters, Basic (0.3% ammonia), Waters XBridge BEH C18 ODB, 5 μm, 30×100 mm column 25-100% MeCN in water) to afford the title compound (FH-2) (5 mg, 0.01 mmol, 10%, 99% Purity) as a white solid. m/z 401.2 (M+H)⁺ (ES+). ¹H NMR (400 MHz, DMSO) δ 8.41 (s, 1H), 8.14-8.10 (m, 1H), 7.98 (d, J=1.9 Hz, 1H), 7.78 (dd, J=8.6, 5.6 Hz, 1H), 7.60 (td, J=8.6, 2.8 Hz, 1H), 7.55-7.49 (m, 2H), 7.41-7.31 (m, 2H), 7.27 (d, J=7.7 Hz, 1H), 7.08 (dd, J=7.8, 1.3 Hz, 1H), 4.02 (s, 3H), 3.13 (s, 3H).

### Example 232: (7-Cyclopropyl-2-(4′-fluoro-2′-(4-methyl-4H-1,2,4-triazol-3-yl)-[1,1′-biphenyl]-3-yl)benzo[d]oxazol-5-yl)methanol (ES-4)

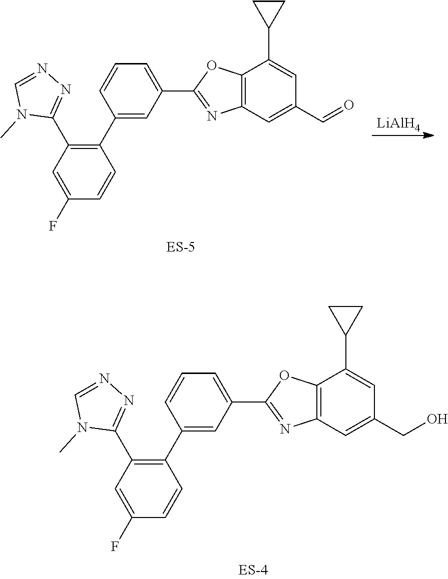

To a solution of intermediate (ES-5) (20 mg, 75% Wt, 1 Eq, 34 μmol) in THF (3.0 mL) was added LiAlH₄ in THF (1.5 mg, 40 μL, 1.0 molar, 1.2 Eq, 40 μmol) dropwise at 0° C. This mixture was stirred at 0° C. for 1 h. The mixture was quenched with NaOH (2 M, 0.5 mL) at 0° C. MgSO₄ was added. The mixture was warmed up to rt, stirred for 15 min, filtered and the solvent was removed in vacuo. The crude product was purified by preparative HPLC (Waters, acidic (0.1% formic acid), acidic, Waters X-Select CSH C18 ODB, 5 μm, 30×100 mm column, 25-100% MeCN in water) to afford the title compound (ES-4) (5.8 mg, 13 μmol, 38%, 99% Purity) as a white solid. m/z 441.1 (M+H)⁺ (ES+). ¹H NMR (400 MHz, DMSO) δ 8.43 (s, 1H), 8.15-8.10 (m, 1H), 7.93-7.90 (m, 1H), 7.79 (dd, J=8.6, 5.6 Hz, 1H), 7.60 (td, J=8.5, 2.9 Hz, 1H), 7.57-7.51 (m, 2H), 7.49-7.45 (m, 1H), 7.35-7.29 (m, 1H), 7.03 (d, J=1.4 Hz, 1H), 5.25 (t, J=5.8 Hz, 1H), 4.56 (d, J=5.8 Hz, 2H), 3.17 (s, 3H), 2.31-2.27 (m, 1H), 1.15-1.08 (m, 2H), 1.03-0.97 (m, 2H).

### Example 234: 2-Ethoxy-N-((2-(4′-fluoro-2′-(4-methyl-4H-1,2,4-triazol-3-yl)-[1,1′-biphenyl]-3-yl)-7-(trifluoromethyl)benzo[d]oxazol-5-yl)methyl)ethan-1-amine (FI-2)

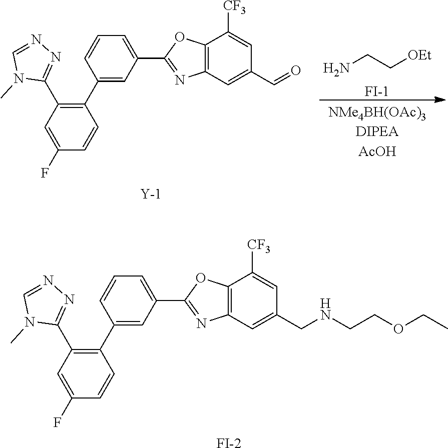

To one well of a 96-well plate were added 2-ethoxyethan-1-amine (FI-1) (7.5 mg, 1.2 Eq, 84 μmol) followed by a solution of intermediate (Y-1) (30 mg, 1 Eq, 70 μmol) in DMF (0.5 mL). DIPEA (18 mg, 24 μL, 2 Eq, 0.14 mmol) was added and the mixture shaken for 30 min. A solution of NMe₄BH(OAc)₃ (46 mg, 2.5 Eq, 0.18 mmol) in DMF (0.5 mL) and AcOH (11 mg, 10 μL, 2.5 Eq, 0.18 mmol) was added and the plate was shaken at rt for 24 h. Sodium carbonate (sat, aq, 6 μL) was added and the crude mixture directly purified by preparative HPLC (Waters, basic (0.3% ammonia), basic, Waters XBridge BEH C18 ODB, 5 μm, 30×100 mm column, MeCN/water) to afford the title compound (FI-2) (7.6 mg, 19%, 98% Purity) as a white solid. m/z 540.2 (M+H)⁺ (ES+). ¹H NMR (400 MHz, DMSO) δ 8.42 (s, 1H), 8.13 (dt, J=7.9, 1.4 Hz, 1H), 8.06 (s, 1H), 7.91 (t, J=1.8 Hz, 1H), 7.80-7.74 (m, 2H), 7.63-7.53 (m, 3H), 7.38 (dt, J=7.8, 1.4 Hz, 1H), 3.90 (s, 2H), 3.47-3.38 (m, 4H), 3.20 (s, 3H), 2.65 (s, 2H), 2.37-2.29 (m, 1H), 1.10 (t, J=7.0 Hz, 3H).

### Example 235: (1S,2R)-2-(((7-(1,1-Difluoroethyl)-2-(4′-fluoro-2′-(4-methyl-4H-1,2,4-triazol-3-yl)-[1,1′-biphenyl]-3-yl)benzo[d]oxazol-5-yl)methyl)amino)cyclopentan-1-ol (FJ-2)

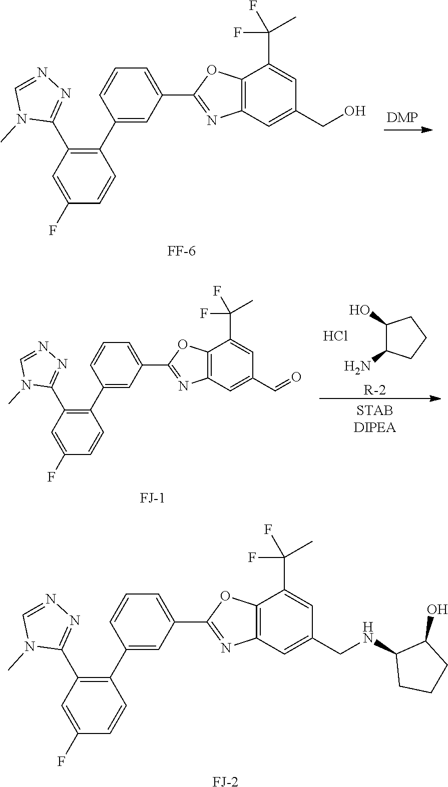

Step 1: 7-(1,1-Difluoroethyl)-2-(4′-fluoro-2′-(4-methyl-4H-1,2,4-triazol-3-yl)-[1,1′-biphenyl]-3-yl)benzo[d]oxazole-5-carbaldehyde (FJ-1). To a stirred solution of intermediate (FF-6) (17.9 mg, 97% Wt, 1 Eq, 37.4 μmol) in dry DCM (3.0 mL) at rt was added Dess-Martin periodinane (31.5 mg, 1.99 Eq, 74.3 μmol). The reaction mixture was stirred at rt for 1 h. Additional Dess-Martin periodinane (34.7 mg, 2.19 Eq, 81.8 μmol) was added and the resulting mixture was stirred at rt overnight. The reaction mixture was diluted with DCM (5 mL), washed with sat. NaHCO₃ (5 mL) and brine (5 mL). The organic layer was dried (Na₂SO₄), filtered and concentrated in vacuo. The crude product was purified by chromatography on silica gel (4 g cartridge, 0-10% MeOH/DCM) to afford the sub-title compound (FJ-1) (18.6 mg, 32 μmol, 86%, 80% Purity) as a pale-yellow solid. m/z 463.0 (M+H)⁺ (ES+). ¹H NMR (400 MHz, MeOD) δ 10.12 (s, 1H), 8.40 (s, 1H), 8.39-8.37 (m, 1H), 8.26 (d, J=7.8 Hz, 1H), 8.16-8.13 (m, 1H), 8.05 (t, J=1.8 Hz, 1H), 7.79 (dd, J=8.6, 5.5 Hz, 1H), 7.64-7.43 (m, 4H), 3.24 (s, 3H), 2.23 (t, J=18.6 Hz, 3H).

Step 2: (1S,2R)-2-(((7-(1,1-Difluoroethyl)-2-(4′-fluoro-2′-(4-methyl-4H-1,2,4-triazol-3-yl)-[1,1′-biphenyl]-3-yl)benzo[d]oxazol-5-yl)methyl)amino)cyclopentan-1-ol (FJ-2). A solution of the product from step 1 above (FJ-1) (18.6 mg, 80% Wt, 1 Eq, 32.2 μmol), (1S,2R)-2-aminocyclopentan-1-ol, HCl (R-2) (10.3 mg, 2.33 Eq, 74.9 μmol) and DIPEA (26 mg, 35 μL, 6.2 Eq, 0.20 mmol) in CHCl₃ (3.0 mL) was stirred 1 h at rt. NaBH(OAc)₃ (28.5 mg, 97% Wt, 4.05 Eq, 130 μmol) was added and the reaction mixture was stirred at rt overnight. Additional NaBH(OAc)₃ (33.1 mg, 97% Wt, 4.71 Eq, 151 μmol) was added and the resulting mixture was stirred at rt for 2 h. The reaction mixture was diluted with EtOAc (2 mL), washed with sat. NaHCO₃ (2 mL) and brine (2 mL), dried (Na₂SO₄), filtered and concentrated in vacuo. The crude product was purified by chromatography on silica gel (4 g cartridge, 0-10% MeOH/DCM) to afford the title compound (FJ-2) (12.4 mg, 22 μmol, 70%, 99% Purity) as a pale-yellow solid. m/z 548.3 (M+H)⁺ (ES+). ¹H NMR (400 MHz, DMSO) δ 8.41 (s, 1H), 8.16-8.12 (m, 1H), 7.91-7.89 (m, 1H), 7.89-7.87 (m, 1H), 7.78 (dd, J=8.6, 5.6 Hz, 1H), 7.64-7.53 (m, 4H), 7.42-7.38 (m, 1H), 4.41-4.36 (m, 1H), 3.99-3.94 (m, 1H), 3.89 (d, J=13.6 Hz, 1H), 3.86 (d, J=13.6 Hz, 1H), 3.19 (s, 3H), 2.22 (t, J=19.2 Hz, 3H), 1.75-1.56 (m, 4H), 1.46-1.36 (m, 2H). 2× Exchangeable protons not observed. ¹⁹F NMR (376 MHz, DMSO) δ −83.59 (q, J=18.5 Hz), −113.66-−113.79 (m).

### Example 236: N-((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)-2-(2-methoxyethoxy)ethan-1-amine (FK-2)

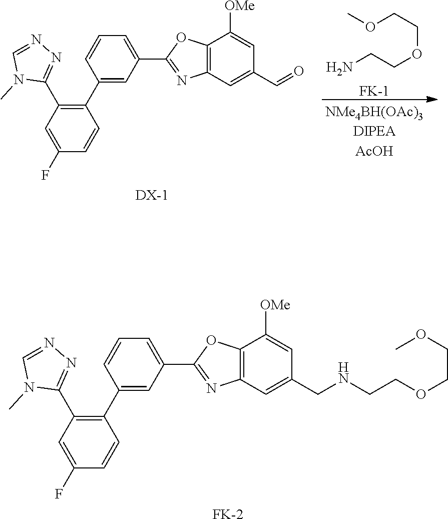

To one well of a 96-well plate were added 2-(2-methoxyethoxy)ethan-1-amine (FK-1) (10 mg, 1.2 Eq, 84 μmol) followed by a solution of intermediate (DX-1) (30 mg, 1 Eq, 70 μmol) in DMF (0.5 mL). DIPEA (18 mg, 24 μL, 2 Eq, 0.14 mmol) was added and the mixture shaken for 30 min. A solution of NMe₄BH(OAc)₃ (46 mg, 2.5 Eq, 0.18 mmol) in DMF (0.5 mL) and AcOH (11 mg, 10 μL, 2.5 Eq, 0.18 mmol) was added and the plate was shaken at rt for 24 h. Sodium carbonate (sat, aq, 6 μL) was added and the crude mixture directly purified by preparative HPLC (Waters, basic (0.3% ammonia), basic, Waters XBridge BEH C18 ODB, 5 μm, 30×100 mm column, MeCN/water) to afford the title compound (FK-2) (9.3 mg, 23%, 99% Purity) as a white solid. m/z 570.2 (M+H)⁺ (ES+). ¹H NMR (400 MHz, DMSO) δ 8.42 (s, 1H), 8.13 (dt, J=7.8, 1.4 Hz, 1H), 8.06 (s, 1H), 7.91 (t, J=1.8 Hz, 1H), 7.80-7.74 (m, 2H), 7.64-7.53 (m, 3H), 7.38 (dt, J=7.8, 1.4 Hz, 1H), 3.90 (s, 2H), 3.52-3.45 (m, 4H), 3.45-3.40 (m, 2H), 3.23 (s, 3H), 3.20 (s, 3H), 2.67-2.63 (m, 2H). NH not observed.

### Example 237: N-((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)propan-1-amine (FL-2)

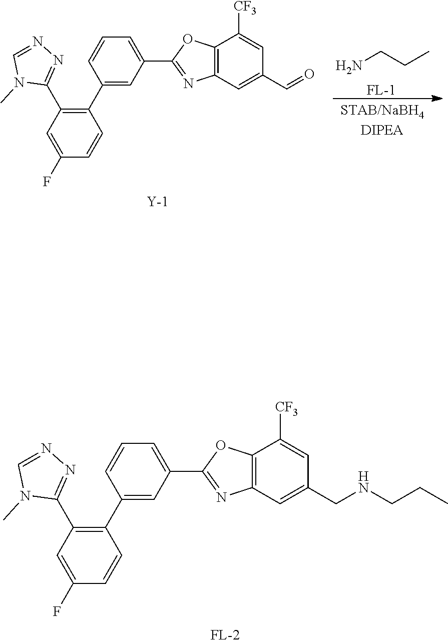

NaBH(OAc)₃ (37 mg, 3.0 Eq, 0.17 mmol) was added to a stirred mixture of intermediate (Y-1) (30 mg, 90% Wt, 1 Eq, 58 μmol), propylamine (FL-1) (6.8 mg, 9.5 μL, 2.0 Eq, 0.12 mmol) and DIPEA (22 mg, 30 μL, 3.0 Eq, 0.17 mmol) in CHCl₃ (3 mL). The reaction mixture was stirred at rt overnight. NaBH₄ (11 mg, 5.0 Eq, 0.29 mmol) was added to the reaction mixture and stirring continued at rt overnight. Additional NaBH₄ (11 mg, 5.0 Eq, 0.29 mmol) was added to the reaction mixture and stirring continued at rt overnight. The reaction mixture was diluted with DCM (4 mL) and washed with sat. aq. NaHCO₃ (10 mL). The organic phase was separated and evaporated to afford the crude product, which was purified by preparative HPLC (Waters, Acidic (0.1% formic acid), Acidic, Waters X-Select CSH C18 OD, 5 μm, 30×100 mm column, 10-100% MeCN in water) to afford the title compound FL-2) (10 mg, 19 μmol, 33%, 98% Purity) as a colourless foam. m/z 510.3 (M+H)⁺ (ES+). ¹H NMR (400 MHz, DMSO) δ 8.42 (s, 1H), 8.20 (s, 1H), 8.13 (dt, J=7.8, 1.4 Hz, 1H), 8.08 (s, 1H), 7.91 (t, J=1.8 Hz, 1H), 7.82-7.73 (m, 2H), 7.65-7.51 (m, 3H), 7.38 (dt, J=7.9, 1.3 Hz, 1H), 3.92 (s, 2H), 3.21 (s, 3H), 1.47 (h, J=7.3 Hz, 2H), 0.88 (t, J=7.4 Hz, 3H). Missing CH₂ under solvent DMSO peak.

### Example 238: N-((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)spiro[3.3]heptan-2-amine (FM-2)

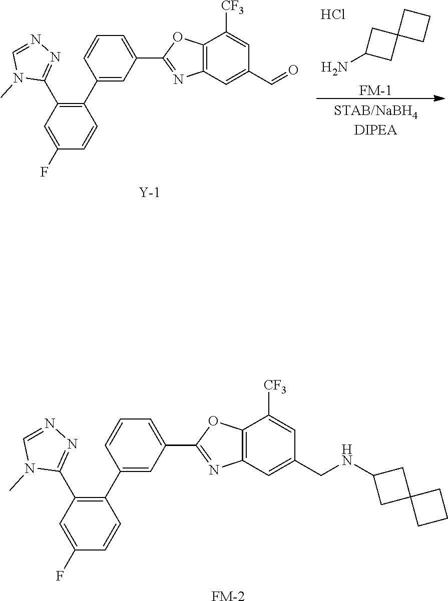

NaBH(OAc)₃ (37 mg, 3.0 Eq, 0.17 mmol) was added to a stirred mixture of intermediate (Y-1) (30 mg, 90% Wt, 1 Eq, 58 μmol), 2-aminospiro[3.3]heptane, HCl (FM-1) (17 mg, 2.0 Eq, 0.12 mmol) and DIPEA (22 mg, 30 μL, 3.0 Eq, 0.17 mmol) in CHCl₃ (3 mL). The reaction mixture was stirred at rt overnight. NaBH₄ (11 mg, 5.0 Eq, 0.29 mmol) was added to the reaction mixture and stirring continued at rt overnight. Additional NaBH₄ (11 mg, 5.0 Eq, 0.29 mmol) was added to the reaction mixture and stirring continued at rt overnight. The reaction mixture was diluted with DCM (4 mL) and washed with sat. aq. NaHCO₃ (10 mL). The organic phase was separated and evaporated to afford the crude product, which was purified by preparative HPLC (Waters, Basic (0.1% ammonia), Basic, Waters X-Bridge Prep-C18, 5 μm, 30×100 mm column, 50-100% MeCN in water) to afford the title compound (FM-2) (16 mg, 27 μmol, 47%, 95% Purity) as a colourless foam. m/z 562.4 (M+H)⁺ (ES+). ¹H NMR (400 MHz, DMSO) δ 8.42 (s, 1H), 8.17-8.09 (m, 1H), 8.03 (s, 1H), 7.91 (t, J=1.8 Hz, 1H), 7.81-7.72 (m, 2H), 7.65-7.51 (m, 3H), 7.37 (dt, J=7.8, 1.6 Hz, 1H), 3.77 (s, 2H), 3.29 (s, 1H), 3.20 (s, 3H), 3.08-2.96 (m, 1H), 2.20-2.11 (m, 2H), 1.95-1.81 (m, 4H), 1.81-1.70 (m, 2H), 1.70-1.60 (m, 2H).

### Example 239: 1-Cyclopentyl-N-((2-(4′-fluoro-2′-(4-methyl-4H-1,2,4-triazol-3-yl)-[1,1′-biphenyl]-3-yl)-7-methoxybenzo[d]oxazol-5-yl)methyl)methanamine (FN-1)

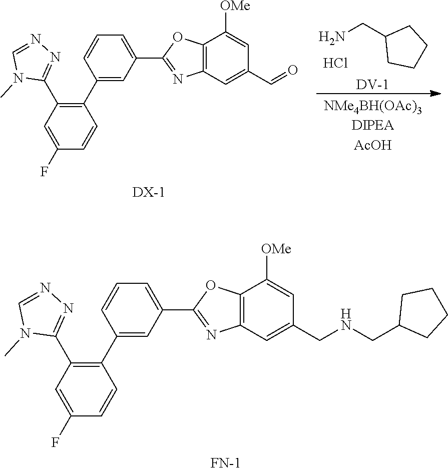

To one well of a 96-well plate were added cyclopentylmethanamine (DV-1) (9.7 mg, 1.2 Eq, 84 μmol) followed by a solution of intermediate (DX-1) (30 mg, 1 Eq, 70 μmol) in DMF (0.5 mL). DIPEA (18 mg, 24 μL, 2 Eq, 0.14 mmol) was added and the mixture shaken for 30 min. A solution of NMe₄BH(OAc)₃ (46 mg, 2.5 Eq, 0.18 mmol) in DMF (0.5 mL) and AcOH (11 mg, 10 μL, 2.5 Eq, 0.18 mmol) was added and the plate was shaken at rt for 24 h. Sodium carbonate (sat, aq, 6 μL) was added and the crude mixture directly purified by preparative HPLC (Waters, basic (0.3% ammonia), basic, Waters XBridge BEH C18 ODB, 5 μm, 30×100 mm column, MeCN/water) to afford the title compound (FN-1) (9.7 mg, 27%, 98% Purity) as a white solid. m/z 512.3 (M+H)⁺ (ES+) ¹H NMR (400 MHz, DMSO) δ 8.41 (s, 1H), 8.10 (dt, J=7.9, 1.4 Hz, 1H), 7.96 (t, J=1.8 Hz, 1H), 7.78 (dd, J=8.6, 5.6 Hz, 1H), 7.60 (td, J=8.6, 2.8 Hz, 1H), 7.55-7.49 (m, 2H), 7.31 (d, J=1.3 Hz, 1H), 7.26 (dt, J=7.9, 1.4 Hz, 1H), 7.08 (d, J=1.3 Hz, 1H), 4.00 (s, 3H), 3.79 (s, 2H), 3.13 (s, 3H), 2.40 (d, J=7.1 Hz, 2H), 2.17-2.05 (m, 1H), 1.98 (p, J=7.5 Hz, 1H), 1.75-1.65 (m, 2H), 1.59-1.42 (m, 4H), 1.22-1.10 (m, 2H).

### Example 240: N-((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)-3-methylbutan-1-amine (FO-2)

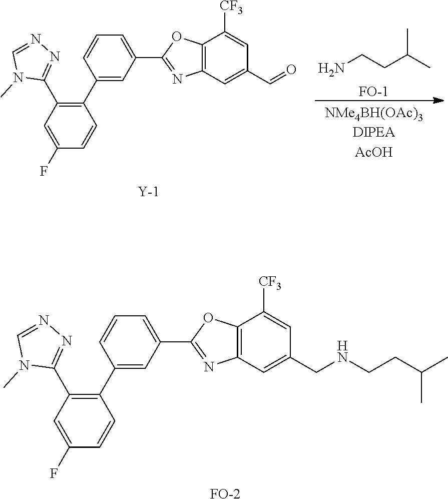

To one well of a 96-well plate were added 3-methylbutan-1-amine (FO-1) (9.7 mg, 1.2 Eq, 84 μmol) followed by a solution of intermediate (Y-1) (30 mg, 1 Eq, 70 μmol) in DMF (0.5 mL). DIPEA (18 mg, 24 μL, 2 Eq, 0.14 mmol) was added and the mixture shaken for 30 min. A solution of NMe₄BH(OAc)₃ (46 mg, 2.5 Eq, 0.18 mmol) in DMF (0.5 mL) and AcOH (11 mg, 10 μL, 2.5 Eq, 0.18 mmol) was added and the plate was shaken at rt for 24 h. Sodium carbonate (sat, aq, 6 μL) was added and the crude mixture directly purified by preparative HPLC (Waters, basic (0.3% ammonia), basic, Waters XBridge BEH C18 ODB, 5 μm, 30×100 mm column, MeCN/water) to afford the title compound (FO-2) (9.5 mg, 25%, 95% Purity) as a white solid. m/z 538.2 (M+H)⁺ (ES+). ¹H NMR (400 MHz, DMSO) δ 8.42 (s, 1H), 8.13 (dt, J=7.9, 1.4 Hz, 1H), 8.05 (s, 1H), 7.91 (t, J=1.8 Hz, 1H), 7.79-7.75 (m, 2H), 7.64-7.53 (m, 3H), 7.37 (dt, J=7.9, 1.4 Hz, 1H), 3.87 (s, 2H), 3.29 (s, 1H), 3.20 (s, 3H), 3.09 (s, 1H), 2.30-2.20 (m, 1H), 1.64 (dp, J=13.4, 6.7 Hz, 1H), 1.33 (q, J=7.0 Hz, 2H), 0.84 (d, J=6.6 Hz, 6H).

### Example 241: (1R,2S)—N-((2-(4′-Fluoro-2′-(4-methyl-4H-1,2,4-triazol-3-yl)-[1,1′-biphenyl]-3-yl)-7-methoxybenzo[d]oxazol-5-yl)methyl)-2-methoxycyclopentan-1-amine (FP-1)

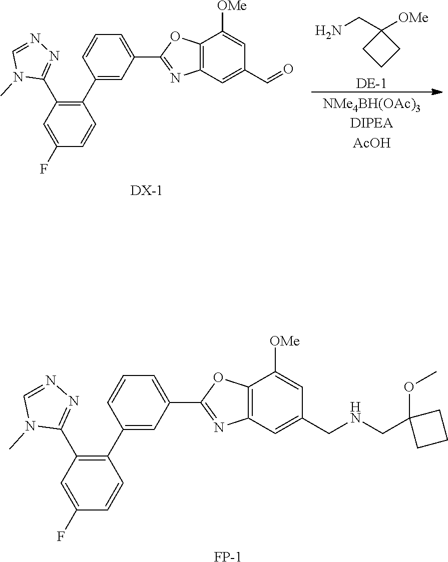

To one well of a 96-well plate were added (1-methoxycyclobutyl)methanamine (DE-1) (9.7 mg, 1.2 Eq, 84 μmol) followed by a solution of intermediate (DX-1) (30 mg, 1 Eq, 70 μmol) in DMF (0.5 mL). DIPEA (18 mg, 24 μL, 2 Eq, 0.14 mmol) was added and the mixture shaken for 30 min. A solution of NMe₄BH(OAc)₃ (46 mg, 2.5 Eq, 0.18 mmol) in DMF (0.5 mL) and AcOH (11 mg, 10 μL, 2.5 Eq, 0.18 mmol) was added and the plate was shaken at rt for 24 h. Sodium carbonate (sat, aq, 6 μL) was added and the crude mixture directly purified by preparative HPLC (Waters, basic (0.3% ammonia), basic, Waters XBridge BEH C18 ODB, 5 μm, 30×100 mm column, MeCN/water) to afford the title compound (FP-1) (8.4 mg, 22%, 99% Purity) as a white solid. m/z 528.3 (M+H)⁺ (ES+). ¹H NMR (400 MHz, DMSO) δ 8.41 (s, 1H), 8.10 (dt, J=8.0, 1.3 Hz, 1H), 7.97 (t, J=1.8 Hz, 1H), 7.78 (dd, J=8.6, 5.6 Hz, 1H), 7.60 (td, J=8.5, 2.8 Hz, 1H), 7.54-7.49 (m, 2H), 7.33 (d, J=1.2 Hz, 1H), 7.29-7.24 (m, 1H), 7.12 (d, J=1.3 Hz, 1H), 4.00 (s, 3H), 3.85 (s, 2H), 3.13 (s, 3H), 3.02 (s, 3H), 2.62 (s, 2H), 1.97 (dd, J=10.7, 7.9 Hz, 2H), 1.91-1.83 (m, 2H), 1.70-1.59 (m, 1H), 1.53-1.40 (m, 1H). One exchangeable proton not observed.

### Example 242: (1R,2S)—N-((2-(4′-Fluoro-2′-(4-methyl-4H-1,2,4-triazol-3-yl)-[1,1′-biphenyl]-3-yl)-7-methoxybenzo[d]oxazol-5-yl)methyl)-2-methoxycyclopentan-1-amine (FQ-1)

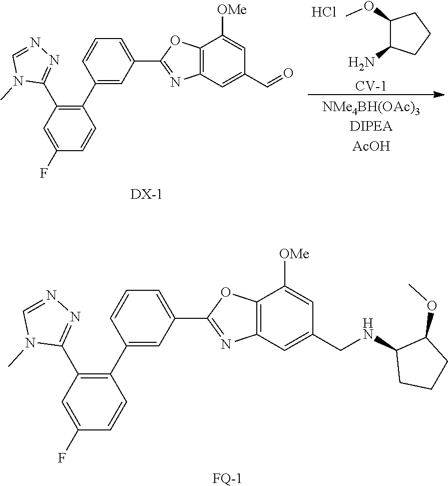

To one well of a 96-well plate were added (1R,2S)-2-methoxycyclopentan-1-amine, HCl (CV-1) (12.7 mg, 1.2 Eq, 84 μmol) followed by a solution intermediate (DX-1) (30 mg, 1 Eq, 70 μmol) in DMF (0.5 mL). DIPEA (18 mg, 24 μL, 2 Eq, 0.14 mmol) was added and the mixture shaken for 30 min. A solution of NMe₄BH(OAc)₃ (46 mg, 2.5 Eq, 0.18 mmol) in DMF (0.5 mL) and AcOH (11 mg, 10 μL, 2.5 Eq, 0.18 mmol) was added and the plate was shaken at rt for 24 h. Sodium carbonate (sat, aq, 6 μL) was added and the crude mixture directly purified by preparative HPLC (Waters, basic (0.3% ammonia), basic, Waters XBridge BEH C18 ODB, 5 μm, 30×100 mm column, MeCN/water) to afford the title compound (FQ-1) (4.2 mg, 11%, 98% Purity) as a white solid. m/z 528.2 (M+H)⁺ (ES+)¹H NMR (400 MHz, DMSO) δ 8.41 (s, 1H), 8.10 (dt, J=7.8, 1.4 Hz, 1H), 7.96 (t, J=1.8 Hz, 1H), 7.78 (dd, J=8.7, 5.6 Hz, 1H), 7.59 (td, J=8.5, 2.8 Hz, 1H), 7.55-7.49 (m, 2H), 7.32 (d, J=1.3 Hz, 1H), 7.26 (dt, J=8.0, 1.3 Hz, 1H), 7.08 (d, J=1.3 Hz, 1H), 4.01 (s, 3H), 3.82 (d, J=5.1 Hz, 2H), 3.61-3.57 (m, 1H), 3.23 (s, 3H), 3.13 (s, 3H), 2.94-2.86 (m, 1H), 1.97-1.90 (m, 1H), 1.74-1.56 (m, 4H), 1.49-1.38 (m, 2H).

### Example 243: (R)-1-(2-(4′-Fluoro-2′-(4-methyl-4H-1,2,4-triazol-3-yl)-[1,1′-biphenyl]-3-yl)-7-methoxybenzo[d]oxazol-5-yl)-N-((tetrahydrofuran-2-yl)methyl)methanamine (FR-1)

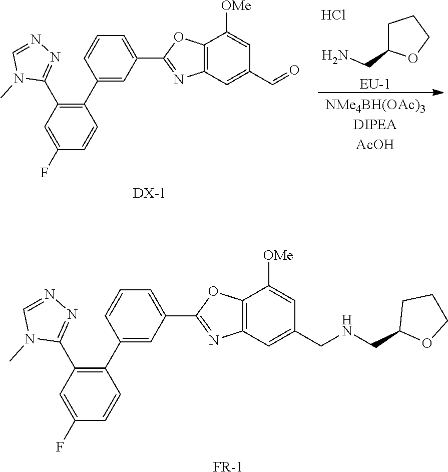

To one well of a 96-well plate were added (R)-(tetrahydrofuran-2-yl)methanamine, HCl (EU-1) (11.6 mg, 1.2 Eq, 84 μmol) followed by a solution of intermediate (DX-1) (30 mg, 1 Eq, 70 μmol) in DMF (0.5 mL). DIPEA (18 mg, 24 μL, 2 Eq, 0.14 mmol) was added and the mixture shaken for 30 min. A solution of NMe₄BH(OAc)₃ (46 mg, 2.5 Eq, 0.18 mmol) in DMF (0.5 mL) and AcOH (11 mg, 10 μL, 2.5 Eq, 0.18 mmol) was added and the plate was shaken at rt for 24 h. Sodium carbonate (sat, aq, 6 μL) was added and the crude mixture directly purified by preparative HPLC (Waters, basic (0.3% ammonia), basic, Waters XBridge BEH C18 ODB, 5 μm, 30×100 mm column, MeCN/water) to afford the title compound (FR-1) (15.1 mg, 42%, 99% Purity) as a white solid. m/z 514.2 (M+H)⁺ (ES+). ¹H NMR (400 MHz, DMSO) δ 8.41 (s, 1H), 8.10 (d, J=7.9 Hz, 1H), 7.96 (t, J=1.8 Hz, 1H), 7.81-7.75 (m, 1H), 7.60 (td, J=8.6, 2.9 Hz, 1H), 7.55-7.49 (m, 2H), 7.31 (d, J=1.3 Hz, 1H), 7.28-7.24 (m, 1H), 7.07 (d, J=1.3 Hz, 1H), 4.01 (s, 3H), 3.93-3.85 (m, 1H), 3.82 (s, 2H), 3.77-3.69 (m, 1H), 3.64-3.56 (m, 1H), 3.13 (s, 3H), 1.95-1.85 (m, 1H), 1.83-1.73 (m, 2H), 1.58-1.48 (m, 1H). Two protons masked by DMSO peak and one exchangeable not observed.

### Example 244: (S)-1-(2-(4′-Fluoro-2′-(4-methyl-4H-1,2,4-triazol-3-yl)-[1,1′-biphenyl]-3-yl)-7-methoxybenzo[d]oxazol-5-yl)-N-((tetrahydrofuran-2-yl)methyl)methanamine (FS-2)

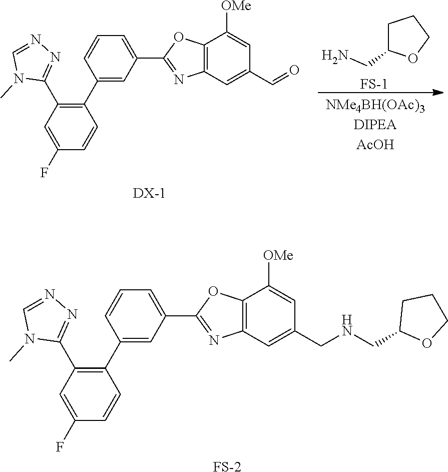

To one well of a 96-well plate were added (S)-(tetrahydrofuran-2-yl)methanamine (FS-1) (8.5 mg, 1.2 Eq, 84 μmol) followed by a solution of intermediate (DX-1) (30 mg, 1 Eq, 70 μmol) in DMF (0.5 mL). DIPEA (18 mg, 24 μL, 2 Eq, 0.14 mmol) was added and the mixture shaken for 30 min. A solution of NMe₄BH(OAc)₃ (46 mg, 2.5 Eq, 0.18 mmol) in DMF (0.5 mL) and AcOH (11 mg, 10 μL, 2.5 Eq, 0.18 mmol) was added and the plate was shaken at rt for 24 h. Sodium carbonate (sat, aq, 6 μL) was added and the crude mixture directly purified by preparative HPLC (Waters, basic (0.3% ammonia), basic, Waters XBridge BEH C18 ODB, 5 μm, 30×100 mm column, MeCN/water) to afford the title compound (FS-2) (6.9 mg, 19%, 99% Purity) as a white solid. m/z 514.2 (M+H)⁺ (ES+). ¹H NMR (400 MHz, DMSO) δ 8.41 (s, 1H), 8.13-8.08 (m, 1H), 7.96 (t, J=1.8 Hz, 1H), 7.78 (dd, J=8.6, 5.7 Hz, 1H), 7.60 (td, J=8.6, 2.8 Hz, 1H), 7.55-7.48 (m, 2H), 7.31 (s, 1H), 7.29-7.24 (m, 1H), 7.07 (d, J=1.3 Hz, 1H), 4.01 (s, 3H), 3.92-3.85 (m, 1H), 3.82 (s, 2H), 3.76-3.69 (m, 1H), 3.60 (q, J=7.3 Hz, 1H), 3.13 (s, 3H), 1.95-1.84 (m, 1H), 1.83-1.73 (m, 2H), 1.58-1.47 (m, 1H). 3H under DMSO.

### Example 245: 1-Cyclobutyl-N-((7-cyclopropyl-2-(4′-fluoro-2′-(4-methyl-4H-1,2,4-triazol-3-yl)-[1,1′-biphenyl]-3-yl)benzo[d]oxazol-5-yl)methyl)methanamine (FT-1)

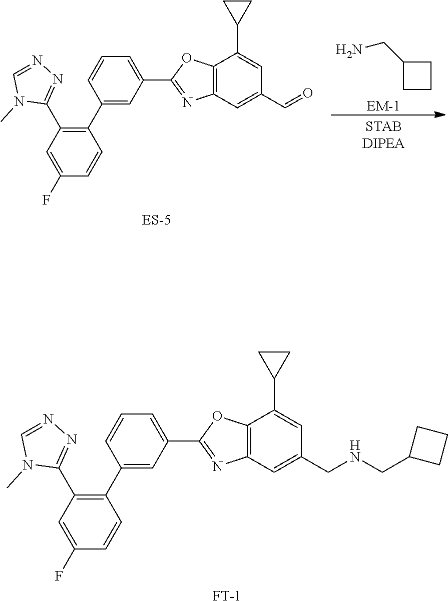

A solution of intermediate (ES-5) (30.3 mg, 75% Wt, 1 Eq, 51.8 μmol), cyclobutylmethanamine (EM-1) (13 mg, 3.0 Eq, 0.16 mmol), and DIPEA (22 mg, 30 μL, 3.3 Eq, 0.17 mmol) in CHCl₃ (3.0 mL) was stirred 1 h at rt. NaBH(OAc)₃ (37.1 mg, 97% Wt, 3.28 Eq, 170 μmol) was added and the reaction mixture was stirred at rt overnight. Additional NaBH(OAc)₃ (41.5 mg, 97% Wt, 3.66 Eq, 190 μmol) was added and the resulting mixture was stirred at rt for 4 h. The reaction mixture was diluted with EtOAc (2 mL), washed with sat. NaHCO₃ (2 mL) and brine (2 mL), dried (Na₂SO₄), filtered and concentrated in vacuo. The crude product was purified by chromatography on silica gel (4 g cartridge, 0-20% MeOH/DCM) to afford the desired product at ˜80% purity. The crude product was purified by preparative HPLC (Waters, Basic (0.1% ammonia), Basic, Waters X-Bridge BEH C18 ODB, 5 μm, 30×100 mm column, 40-100% MeCN in water) to afford the title compound (FT-1) (8.4 mg, 16 μmol, 32%, 99% Purity) as a white solid. m/z 508.2 (M+H)⁺ (ES+). ¹H NMR (400 MHz, DMSO) δ 8.43 (s, 1H), 8.15-8.08 (m, 1H), 7.90 (t, J=1.8 Hz, 1H), 7.78 (dd, J=8.6, 5.7 Hz, 1H), 7.65-7.50 (m, 3H), 7.49 (d, J=1.5 Hz, 1H), 7.37-7.30 (m, 1H), 7.05 (d, J=1.5 Hz, 1H), 3.75 (s, 2H), 3.29 (s, 2H), 3.16 (s, 3H), 2.45-2.36 (m, 1H), 2.31-2.24 (m, 1H), 2.04-1.92 (m, 2H), 1.87-1.71 (m, 2H), 1.68-1.57 (m, 2H), 1.16-1.06 (m, 2H), 1.04-0.96 (m, 2H). 1 Exchangeable NH proton not observed.

### Example 246: 1-Cyclopropyl-N-((2-(4′-fluoro-2′-(4-methyl-4H-1,2,4-triazol-3-yl)-[1,1′-biphenyl]-3-yl)-7-methoxybenzo[d]oxazol-5-yl)methyl)methanamine (FU-1)

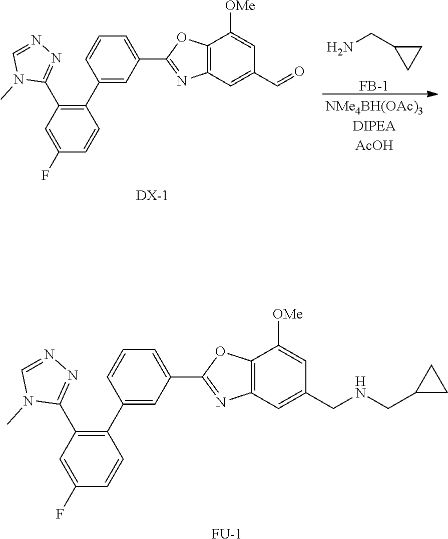

To one well of a 96-well plate were added cyclopropylmethanamine (FB-1) (6.0 mg, 1.2 Eq, 84 μmol) followed by a solution of intermediate (DX-1) (30 mg, 1 Eq, 70 μmol) in DMF (0.5 mL). DIPEA (18 mg, 24 μL, 2 Eq, 0.14 mmol) was added and the mixture shaken for 30 min. A solution of NMe₄BH(OAc)₃ (46 mg, 2.5 Eq, 0.18 mmol) in DMF (0.5 mL) and AcOH (11 mg, 10 μL, 2.5 Eq, 0.18 mmol) was added and the plate was shaken at rt for 24 h. Sodium carbonate (sat, aq, 6 μL) was added and the crude mixture directly purified by preparative HPLC (Waters, basic (0.3% ammonia), basic, Waters XBridge BEH C18 ODB, 5 μm, 30×100 mm column, MeCN/water) to afford the title compound (FU-1) (6.2 mg, 18%, 98% Purity) as a white solid. m/z 484.2 (M+H)⁺ (ES+), ¹H NMR (400 MHz, DMSO) δ 8.41 (s, 1H), 8.10 (dt, J=7.9, 1.3 Hz, 1H), 7.96 (t, J=1.8 Hz, 1H), 7.78 (dd, J=8.7, 5.7 Hz, 1H), 7.59 (td, J=8.5, 2.8 Hz, 1H), 7.55-7.48 (m, 2H), 7.31 (d, J=1.2 Hz, 1H), 7.26 (dt, J=7.8, 1.3 Hz, 1H), 7.08 (d, J=1.3 Hz, 1H), 4.01 (s, 3H), 3.81 (s, 2H), 3.13 (s, 3H), 2.38 (d, J=6.6 Hz, 2H), 0.96-0.85 (m, 1H), 0.43-0.36 (m, 2H), 0.11-0.05 (m, 2H). One exchangeable proton not observed.

### Example 247: rac-(1R,3S)—N-((2-(4′-fluoro-2′-(4-methyl-4H-1,2,4-triazol-3-yl)-[1,1′-biphenyl]-3-yl)-7-(trifluoromethyl)benzo[d]oxazol-5-yl)methyl)-3-methylcyclohexan-1-amine (FV-2) and rac-(1R,3R)—N-((2-(4′-fluoro-2′-(4-methyl-4H-1,2,4-triazol-3-yl)-[1,1′-biphenyl]-3-yl)-7-(trifluoromethyl)benzo[d]oxazol-5-yl)methyl)-3-methylcyclohexan-1-amine (FV-3)

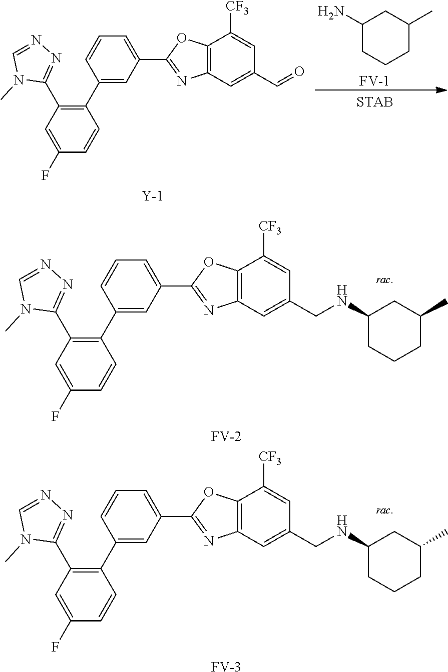

A solution of intermediate (Y-1) (60 mg, 90% Wt, 1 Eq, 0.12 mmol) and 3-methylcyclohexan-1-amine (FV-1) (26 mg, 2 Eq, 0.23 mmol) in CHCl₃ (3 mL) was stirred for 1 h at rt. NaBH(OAc)₃ (74 mg, 3.0 Eq, 0.35 mmol) was added and the reaction mixture was stirred at rt overnight. The reaction mixture was diluted with EtOAc (5 mL) and washed with a sat. aq sol. of NaHCO₃ (5 mL) and brine (5 mL). The organic layer was extracted, dried (MgSO₄), and concentrated in vacuo. The crude was purified by preparative HPLC (Waters, Basic (0.1% ammonia), Waters XBridge BEH C18 ODB, 5 μm, 30×100 mm column 55-100% MeCN in water) to afford the title compounds; Peak 1 (FV-2) (17.29 mg, 29 μmol, 25%, 95% Purity) as a white solid. m/z 564.5 (M+H)⁺ (ES+). ¹H NMR (400 MHz, DMSO) δ 8.42 (s, 1H), 8.13 (d, J=7.8 Hz, 1H), 8.07 (s, 1H), 7.90 (t, J=1.9 Hz, 1H), 7.80-7.75 (m, 2H), 7.64-7.52 (m, 3H), 7.39-7.35 (m, 1H), 3.92 (s, 2H), 3.20 (s, 3H), 2.40-2.30 (m, 1H), 2.20 (s, 1H), 1.95-1.85 (m, 2H), 1.71-1.62 (m, 1H), 1.62-1.53 (m, 1H), 1.39-1.25 (m, 1H), 1.26-1.12 (m, 1H), 0.87 (d, J=6.6 Hz, 3H), 0.83-0.63 (m, 2H). One exchangeable proton not observed. Peak 2 (FV-3) (15.63 mg, 26 μmol, 23%, 95% Purity) as a white solid. m/z 564.3 (M+H)⁺ (ES+). ¹H NMR (400 MHz, DMSO) δ 8.42 (s, 1H), 8.13 (dt, J=7.9, 1.4 Hz, 1H), 8.08 (s, 1H), 7.90 (t, J=1.8 Hz, 1H), 7.81-7.74 (m, 2H), 7.64-7.53 (m, 3H), 7.40-7.35 (m, 1H), 3.88 (s, 2H), 3.20 (s, 3H), 2.78-2.72 (m, 1H), 2.26-2.13 (m, 1H), 1.90-1.78 (m, 1H), 1.66-1.49 (m, 3H), 1.43-1.32 (m, 2H), 1.19-1.11 (m, 1H), 1.01-0.88 (m, 1H), 0.83 (d, J=6.8 Hz, 3H). One exchangeable proton not observed.

### Example 248: (1S,2R)-2-(((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)(methyl)amino)cyclopentan-1-ol (FW-3)

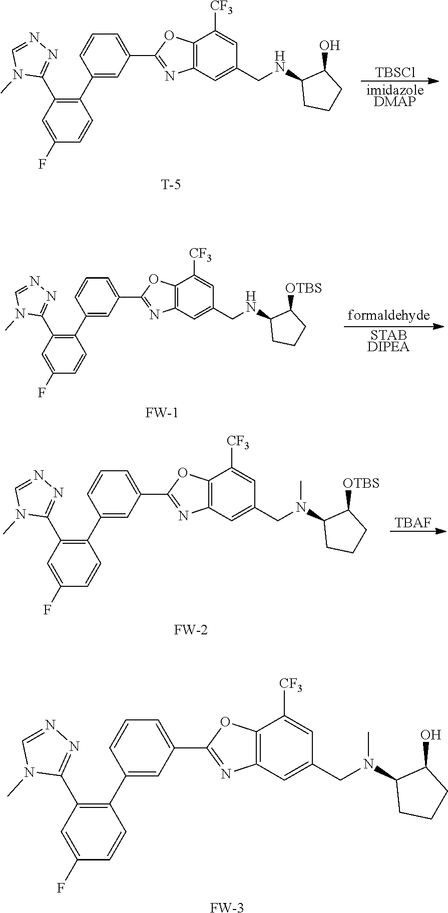

Step 1: (1R,2S)-2-((tert-Butyldimethylsilyl)oxy)-N-((2-(4′-fluoro-2′-(4-methyl-4H-1,2,4-triazol-3-yl)-[1,1′-biphenyl]-3-yl)-7-(trifluoromethyl)benzo[d]oxazol-5-yl)methyl)cyclopentan-1-amine (FW-1). To a stirred solution of compound (T-5) (41.4 mg, 85% Wt, 1 Eq, 63.8 μmol), imidazole (21.5 mg, 4.95 Eq, 316 μmol) and DMAP (0.8 mg, 0.1 Eq, 7 μmol) in dry DMF (0.5 mL) under a nitrogen atmosphere at rt was added TBDMSCl (21.6 mg, 24.6 μL, 2.25 Eq, 143 μmol). The reaction mixture was stirred at rt overnight. The reaction mixture was cooled to rt, diluted with EtOAc (5 mL), washed with 1:1 brine: H₂O (3×5 mL), dried (Na₂SO₄), filtered and concentrated in vacuo. The crude product was purified by chromatography on silica gel (4 g cartridge, 0-10% MeOH/DCM) to afford the sub-title compound (FW-1) (16.6 mg, 24 μmol, 37%, 95% Purity) as a pale-yellow oil. m/z 666.3 (M+H)⁺ (ES+). ¹H NMR (400 MHz, MeOD) δ 8.38 (s, 1H), 8.25-8.18 (m, 1H), 8.09-8.05 (m, 1H), 8.00-7.95 (m, 1H), 7.79 (dd, J=8.6, 5.4 Hz, 1H), 7.75-7.70 (m, 1H), 7.61-7.51 (m, 2H), 7.47 (dd, J=8.7, 2.7 Hz, 1H), 7.43 (d, J=7.8 Hz, 1H), 4.33-4.27 (m, 1H), 3.97 (s, 2H), 3.24 (s, 3H), 3.01-2.92 (m, 1H), 1.91 (dd, J=7.1, 3.3 Hz, 1H), 1.86-1.70 (m, 3H), 1.64-1.49 (m, 2H), 0.91 (s, 9H), 0.12 (s, 6H). 1× Exchangeable NH proton not observed.

Step 2: (1R,2S)-2-((tert-Butyldimethylsilyl)oxy)-N-((2-(4′-fluoro-2′-(4-methyl-4H-1,2,4-triazol-3-yl)-[1,1′-biphenyl]-3-yl)-7-(trifluoromethyl)benzo[d]oxazol-5-yl)methyl)-N-methylcyclopentan-1-amine (FW-2). A solution of the product from step 1 above (FW-1) (16.6 mg, 95% Wt, 1 Eq, 23.7 μmol), formaldehyde solution in H₂O (3.84 mg, 3.53 μL, 37% Wt, 2 Eq, 47.4 μmol) and DIPEA (22 mg, 30 μL, 7.3 Eq, 0.17 mmol) in CHCl₃ (3.0 mL) was stirred 1 h at rt. NaBH(OAc)₃ (24.2 mg, 97% Wt, 4.68 Eq, 111 μmol) was added and the reaction mixture was stirred at rt overnight. The reaction mixture was diluted with EtOAc (2 mL), washed with sat. NaHCO₃ (2 mL) and brine (2 mL), dried (Na₂SO₄), filtered and concentrated in vacuo. The crude product was purified by chromatography on silica gel (4 g cartridge, 0-10% MeOH/DCM) to afford the sub-title compound (FW-2) (13.0 mg, 19 μmol, 78%, 97% Purity) as a yellow oil. m/z 680.3 (M+H)⁺ (ES+). ¹H NMR (400 MHz, MeOD) δ 8.38 (s, 1H), 8.20 (dt, J=7.9, 1.4 Hz, 1H), 8.06 (t, J=1.7 Hz, 1H), 7.93 (s, 1H), 7.81-7.74 (m, 2H), 7.60-7.51 (m, 2H), 7.49-7.39 (m, 2H), 4.41-4.35 (m, 1H), 3.89 (d, J=13.7 Hz, 1H), 3.79 (d, J=13.7 Hz, 1H), 3.24 (s, 3H), 2.61 (td, J=8.7, 3.5 Hz, 1H), 2.23 (s, 3H), 1.94-1.85 (m, 3H), 1.85-1.74 (m, 1H), 1.74-1.58 (m, 2H), 0.95 (s, 9H), 0.12 (s, 3H), 0.09 (s, 3H).

Step 3: (1S,2R)-2-(((2-(4′-fluoro-2′-(4-methyl-4H-1,2,4-triazol-3-yl)-[1,1′-biphenyl]-3-yl)-7-(trifluoromethyl)benzo[d]oxazol-5-yl)methyl)(methyl)amino)cyclopentan-1-ol (FW-3). To a stirred solution of the product from step 2 above (FW-2) (13.0 mg, 97% Wt, 1 Eq, 18.5 μmol) in THF (1.0 mL) at rt was added TBAF in THF (34 mg, 37 μL, 1.0 molar, 2.0 Eq, 37 μmol). The reaction mixture was stirred at rt for 2 h. The volatiles were removed in vacuo. The crude product was purified by chromatography on silica gel (4 g cartridge, 0-10% MeOH/DCM) to recover ˜9 mg SM. The recovered SM was resubjected to deprotection by being redissolved in THF (1.0 mL) and treated with TBAF in THF (9.7 mg, 37 μL, 1.0 molar, 2.0 Eq, 37 μmol). The mixture was stirred at rt for 4 h. Further TBAF in THF (9.7 mg, 37 μL, 1.0 molar, 2.0 Eq, 37 μmol) was added and the reaction mixture was stirred at rt for 3 d. The volatiles were removed in vacuo. The crude product was purified by chromatography on silica gel (4 g cartridge, 0-10% MeOH/DCM) to afford the title compound (FW-3) (6.8 mg, 11 μmol, 62%, 95% Purity) as a white solid. m/z 566.3 (M+H)⁺ (ES+). ¹H NMR (400 MHz, MeOD) δ 8.38 (s, 1H), 8.21 (dt, J=7.9, 1.4 Hz, 1H), 8.06 (t, J=1.8 Hz, 1H), 8.01 (s, 1H), 7.81-7.75 (m, 2H), 7.61-7.51 (m, 2H), 7.49-7.41 (m, 2H), 4.29-4.23 (m, 1H), 3.89 (d, J=13.2 Hz, 1H), 3.76 (d, J=13.4 Hz, 1H), 3.25 (s, 3H), 2.64-2.56 (m, 1H), 2.22 (s, 3H), 1.99-1.89 (m, 2H), 1.89-1.75 (m, 3H), 1.75-1.62 (m, 1H). 1× Exchangeable OH proton not observed.

### Example 249: N-((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)-2-methylpropan-1-amine (FX-2)

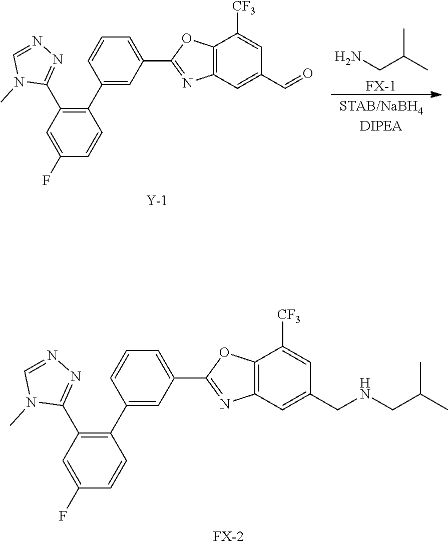

NaBH(OAc)₃ (37 mg, 3.0 Eq, 0.17 mmol) was added to a stirred mixture of intermediate (Y-1) (30 mg, 90% Wt, 1 Eq, 58 μmol), 2-methylpropan-1-amine (FX-1) (8.5 mg, 12 μL, 2.0 Eq, 0.12 mmol) and DIPEA (22 mg, 31 μL, 3.0 Eq, 0.17 mmol) in CHCl₃ (3 mL). The reaction mixture was stirred at rt over the weekend. NaBH₄ (22 mg, 10.0 Eq, 0.58 mmol) was added to the reaction mixture and stirring continued at rt overnight. More NaBH₄ (22 mg, 10.0 Eq, 0.58 mmol) was added to the reaction mixture and stirring continued at 40° C. for 6 h and then at rt overnight. The reaction mixture was diluted with DCM (4 mL) and washed with sat. aq. NaHCO₃ (10 mL). The organic phase was separated and evaporated to afford the crude product. The crude product was purified by preparative HPLC (Waters, acidic (0.10% formic acid), acidic, Waters X-Select CSH C18 ODB prep column, 130A, 5 μm, 30 mm×100 mm column, 15-100% MeCN in water) to afford the title compound (FX-2) (11 mg, 20 μmol, 35%, 97% Purity) as a colourless foam. m/z 524.4 (M+H)⁺ (ES+). ¹H NMR (400 MHz, DMSO) δ 8.42 (s, 1H), 8.13 (dt, J=7.9, 1.3 Hz, 1H), 8.07 (s, 1H), 7.91 (t, J=1.8 Hz, 1H), 7.80-7.75 (m, 2H), 7.65-7.51 (m, 3H), 7.38 (dt, J=7.9, 1.4 Hz, 1H), 3.88 (s, 2H), 3.20 (s, 3H), 2.31 (d, J=6.7 Hz, 2H), 1.76-1.62 (m, 1H), 0.88 (d, J=6.6 Hz, 6H). NH signal not observed.

### Example 250: N-((2-(4′-Fluoro-2′-(4-methyl-4H-1,2,4-triazol-3-yl)-[1,1′-biphenyl]-3-yl)-7-methoxybenzo[d]oxazol-5-yl)methyl)-3-methoxypropan-1-amine (FY-1)

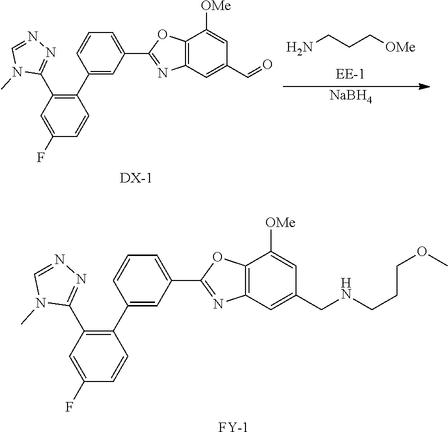

To one well of a 96-well plate were was added 3-methoxypropan-1-amine (EE-1) (7.5 mg, 8.6 μL, 1.2 Eq, 84 μmol) followed by a solution of intermediate (DX-1) (30 mg, 1 Eq, 70 μmol) in DMF (0.5 mL) was added and shaken for 30 min. A solution of NaBH₄ (6.6 mg, 2.5 Eq, 0.18 mmol) in DMF (0.5 mL) was added and the plate was shaken at rt for 24 h. The reaction was quenched with 7 M NH₃ in MeOH (200 μL) and filtered. The crude product was purified by preparative HPLC (Waters, Basic (0.1% ammonium bicarbonate), Basic, Waters X-Bridge Prep-C18, 5 μm, 30×100 mm column, 0-100% MeCN in water) to afford the title compound (FY-1) (4.76 mg, 9.3 μmol, 13%,98% Purity) as a white solid. m/z 502.2 (M+H)⁺ (ES+). 1H NMR (400 MHz, DMSO) δ 8.41 (s, 1H), 8.12-8.09 (m, 1H), 7.96 (t, J=1.8 Hz, 1H), 7.78 (dd, J=8.6, 5.7 Hz, 1H), 7.59 (td, J=8.5, 2.8 Hz, 1H), 7.55-7.49 (m, 2H), 7.31 (d, J=1.3 Hz, 1H), 7.28-7.25 (m, 1H), 7.06 (d, J=1.3 Hz, 1H), 4.01 (s, 3H), 3.77 (s, 2H), 3.37 (t, J=6.4 Hz, 2H), 3.20 (s, 3H), 3.13 (s, 3H), 2.56-2.51 (m, 2H), 1.66 (p, J=6.7 Hz, 2H). One exchangeable proton not observed.

### Example 251: (2-(4′-Fluoro-2′-(4-methyl-4H-1,2,4-triazol-3-yl)-[1,1′-biphenyl]-3-yl)imidazo[1,2-a]pyridin-6-yl)methanol (FZ-5)

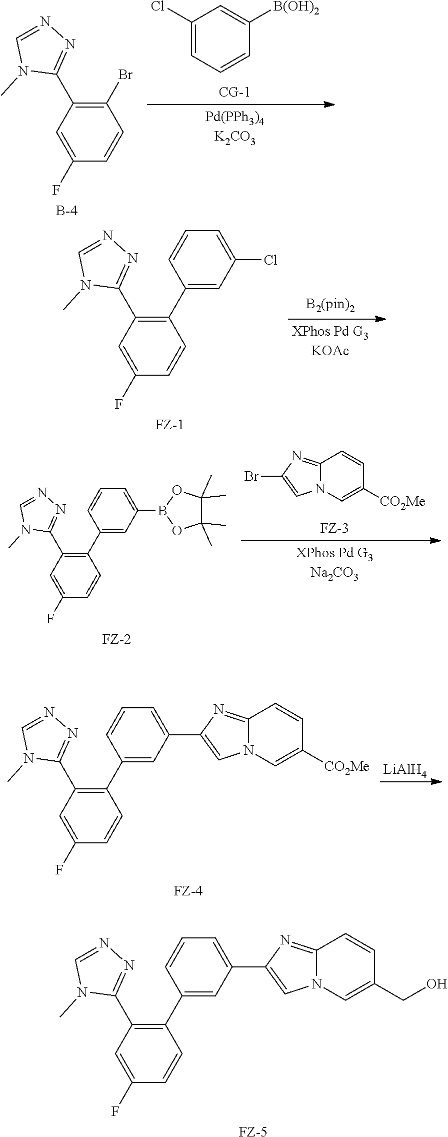

Step 1: 3-(3′-Chloro-4-fluoro-[1,1′-biphenyl]-2-yl)-4-methyl-4H-1,2,4-triazole (FZ-1). To a solution of intermediate (B-5) (500 mg, 100% Wt, 1 Eq, 1.95 mmol) and (3-chlorophenyl)boronic acid (CG-1) (366 mg, 1.2 Eq, 2.34 mmol) in 1,4-dioxane (15.0 mL)) and water (4.5 mL) were added Pd(PPh₃)₄ (226 mg, 0.1 Eq, 195 μmol) and K₂CO₃ (810 mg, 3.0 Eq, 5.86 mmol). The reaction mixture was sparged with nitrogen for 2 min then stirred at 110° C. (microwave) for 2 h. The reaction mixture was filtered through a Celite plug (chased with 20 mL MeOH) and the filtrate concentrated in vacuo. The residue was partitioned between 1:1 EtOAc-DCM (20 mL) and water (20 mL) before being passed through a phase separator. The organic phase was evaporated to dryness to afford the crude product as a dark brown tar. The crude product was purified by chromatography on silica gel (40 g cartridge, 0-10% MeOH/DCM) to afford the sub-title compound (FZ-1) (656 mg, 1.5 mmol, 76%, 65% Purity) as a brown oil which partially crystallized on standing. m/z 287.8 & 288.4 (M+H)⁺ (ES+). ¹H NMR (400 MHz, DMSO) δ 8.45 (s, 1H), 7.70-7.59 (m, 4H), 7.55 (ddd, J=8.5, 6.6, 2.8 Hz, 3H), 7.48 (dd, J=9.1, 2.8 Hz, 1H), 7.39-7.29 (m, 2H), 7.17 (t, J=1.9 Hz, 1H), 7.02 (dt, J=7.5, 1.6 Hz, 1H), 3.12 (s, 3H). Extra aromatic protons attributed to Ph₃PO impurity.

Step 2: 3-(4-Fluoro-3′-(4,4,5,5-tetramethyl-1,3,2-dioxaborolan-2-yl)-[1,1′-biphenyl]-2-yl)-4-methyl-4H-1,2,4-triazole (FZ-2). A mixture of the product from step 1 above (FZ-1) (315 mg, 65% Wt, 1 Eq, 712 μmol), XPhos Pd G3 (30.1 mg, 0.05 Eq, 35.6 μmol), bis(pinacolato)diboron (361 mg, 2 Eq, 1.42 mmol), and potassium acetate (349 mg, 5 Eq, 3.56 mmol) in 1,4-dioxane (6 mL) was sparged with nitrogen for 2 min. and then stirred at 110° C. (microwave) for 1 h. The reaction was stirred at 110° C. (microwave) for another 1 h. The reaction mixture was left to stand a rt over the weekend. More XPhos Pd G3 (30.1 mg, 0.05 Eq, 35.6 μmol) was added to the reaction mixture which was stirred at 110° C. (microwave) for an additional 1 h. The reaction mixture was left to stand at rt overnight. The crude reaction mixture was passed through a Celite plug (chased with 10 mL MeOH) and the filtrate (crude product) adsorbed onto silica-gel. The crude product was purified by chromatography on silica gel (12 g cartridge, 0-100% acetone in iso-hexane) to afford the sub-title compound (FZ-2) (204 mg, 0.51 mmol, 72%, 95% Purity) as a colourless foam. m/z 380.3 (M+H)⁺ (ES+). 1H NMR (400 MHz, DMSO) δ 8.39 (s, 1H), 7.65 (dd, J=8.7, 5.8 Hz, 1H), 7.58 (dt, J=7.4, 1.3 Hz, 1H), 7.53 (td, J=8.5, 2.8 Hz, 1H), 7.45 (dt, J=6.2, 2.9 Hz, 2H), 7.29 (t, J=7.6 Hz, 1H), 7.17-7.06 (m, 1H), 3.02 (s, 3H), 1.28 (s, 12H).

Step 3: Methyl 2-(4′-fluoro-2′-(4-methyl-4H-1,2,4-triazol-3-yl)-[1,1′-biphenyl]-3-yl)imidazo[1,2-a]pyridine-6-carboxylate (FZ-4). A solution of the product from step 2 above (FZ-2) (85.6 mg, 56% Wt, 1 Eq, 126 μmol), methyl-2-bromoimidazo[1,2-a]pyridine-6-carboxylate (FZ-3) (38.5 mg, 1.19 Eq, 151 μmol) and sodium carbonate (42.0 mg, 3.13 Eq, 396 μmol) in 1,4-dioxane (2.0 mL) and water (0.2 mL) was degassed under a nitrogen atmosphere at rt for 10 min. XPhos Pd G3 (7.9 mg, 0.074 Eq, 9.3 μmol) was added and the reaction mixture was heated at 100° C. for 7 d. The reaction mixture was cooled to rt and filtered through a short pad of Celite. The volatiles were removed in vacuo. The crude product was purified by chromatography on silica gel (12 g cartridge, 0-10% MeOH/DCM) to afford the sub-title compound (FZ-4) (48.4 mg, 70 μmol, 56%, 62% Purity) as a yellow solid. m/z 428.2 (M+H)⁺ (ES+). ¹H NMR (400 MHz, DMSO) δ 9.29 (t, J=1.4 Hz, 1H), 8.46 (s, 1H), 8.40 (s, 1H), 7.92-7.88 (m, 1H), 7.79-7.77 (m, 1H), 7.74 (dd, J=8.7, 5.8 Hz, 1H), 7.66-7.64 (m, 2H), 7.62-7.55 (m, 1H), 7.49 (dd, J=9.2, 2.9 Hz, 1H), 7.38 (t, J=7.8 Hz, 1H), 7.02-6.98 (m, 1H), 3.90 (s, 3H), 3.09 (s, 3H).

### Step 4: (2-(4′-Fluoro-2′-(4-methyl-4H-1,2,4-triazol-3-yl)-[1,1′-biphenyl]-3-yl)imidazo[1,2-a]pyridin-6-yl)methanol (FZ-5)

To a solution of the product from step 3 above (FZ-4) (48.4 mg, 62% Wt, 1 Eq, 70.2 μmol) in THF (2.0 mL) was added LiAlH₄ in THF (2.7 mg, 70 μL, 1 molar, 1.0 Eq, 70 μmol) dropwise at 0° C. The mixture was stirred at 0° C. for 1 h. The mixture was quenched with NaOH (2 M, 0.5 mL) at 0° C. Na₂SO₄ was added. The mixture was warmed up to rt, stirred for 15 min, filtered and the volatiles were removed in vacuo. The crude product was purified by chromatography on silica gel (4 g cartridge, 0-10% MeOH/DCM) to afford the title compound (FZ-5) (6.0 mg, 14 μmol, 21%, 96% Purity) as a yellow solid. m/z 400.1 (M+H)⁺ (ES+). ¹H NMR (400 MHz, MeOD) δ 8.42-8.39 (m, 1H), 8.36 (s, 1H), 8.06 (d, J=0.7 Hz, 1H), 7.92-7.87 (m, 1H), 7.79-7.73 (m, 2H), 7.55-7.48 (m, 2H), 7.45-7.38 (m, 2H), 7.34 (dd, J=9.3, 1.6 Hz, 1H), 7.15-7.11 (m, 1H), 4.64 (d, J=1.1 Hz, 2H), 3.10 (s, 3H). 1× Exchangeable OH proton not observed.

### Example 252: 1-((((2-(4′-Fluoro-2′-(4-methyl-4H-1,2,4-triazol-3-yl)-[1,1′-biphenyl]-3-yl)-7-methoxybenzo[d]oxazol-5-yl)methyl)amino)methyl)cyclobutan-1-ol (GA-1)

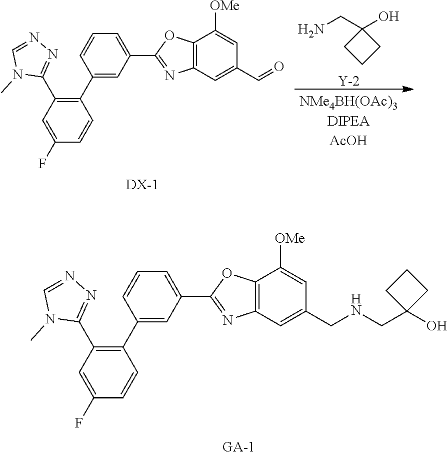

To one well of a 96-well plate were added 1-(aminomethyl)cyclobutan-1-ol (Y-2) (10.6 mg, 1.5 Eq, 0.11 mmol) followed by a solution of intermediate (DX-1) (30 mg, 1 Eq, 70 μmol) in DMF (0.5 mL). DIPEA (18 mg, 24 μL, 2 Eq, 0.14 mmol) was added and the mixture shaken for 30 min. A solution of NMe₄BH(OAc)₃ (46 mg, 2.5 Eq, 0.18 mmol) in DMF (0.5 mL) and AcOH (11 mg, 10 μL, 2.5 Eq, 0.18 mmol) was added and the plate was shaken at rt for 3 d. A solution of NH₃/MeOH (200 μL) was added and the crude mixture directly purified by preparative HPLC (Waters, basic (0.3% ammonia), basic, Waters XBridge BEH C18 ODB, 5 μm, 30×100 mm column, MeCN/water) to afford the title (GA-1) (6.97 mg, 0.011 mmol, 15.2%, 95% Purity) as a white solid. m/z 514.5 (M+H)⁺ (ES+). ¹H NMR (400 MHz, DMSO) δ 8.42 (d, J=3.4 Hz, 1H), 8.11 (dt, J=7.8, 1.4 Hz, 1H), 7.96 (t, J=1.8 Hz, 1H), 7.78 (dd, J=8.6, 5.6 Hz, 1H), 7.59 (td, J=8.6, 2.8 Hz, 1H), 7.54-7.49 (m, 2H), 7.33 (d, J=1.3 Hz, 1H), 7.28-7.24 (m, 1H), 7.10 (d, J=1.3 Hz, 1H), 4.90 (s, 1H), 4.01 (s, 3H), 3.86 (s, 2H), 3.13 (s, 3H), 2.05-1.98 (m, 2H), 1.96-1.85 (m, 2H), 1.67-1.56 (m, 1H), 1.45-1.34 (m, 1H). 3H under DMSO.

### Example 253: 1-(2-(4′-Fluoro-2′-(4-methyl-4H-1,2,4-triazol-3-yl)-[1,1′-biphenyl]-3-yl)-7-(trifluoromethyl)benzo[d]oxazol-5-yl)-N-(oxetan-3-ylmethyl)methanamine (GB-2)

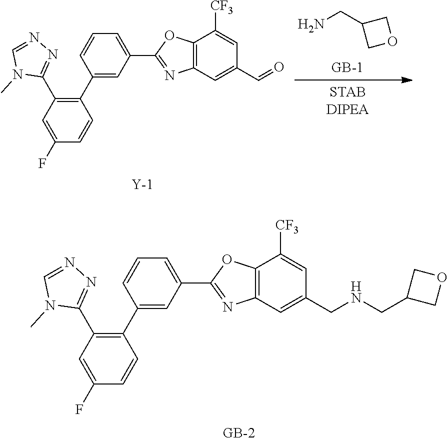

A solution of intermediate (Y-1) (30 mg, 90% Wt, 1 Eq, 58 μmol), DIPEA (22 mg, 30 μL, 3.0 Eq, 0.17 mmol) and oxetan-3-ylmethanamine (GB-1) (11 mg, 90% Wt, 2.0 Eq, 0.12 mmol) in CHCl₃ (3 mL) was stirred for 1 h at rt. NaBH(OAc)₃ (37 mg, 3.0 Eq, 0.17 mmol) was added to the reaction mixture and stirred for 16 h at rt. The reaction mixture was diluted with EtOAc (5 mL) and washed with a sat. aq sol. of NaHCO₃ (5 mL) and brine (5 mL). The organic layer was extracted, dried (MgSO₄), and concentrated in vacuo. The crude product was purified by preparative HPLC (Waters, Basic (0.1% ammonium bicarbonate), Basic, Waters X-Bridge Prep-C18, 5 μm, 30×100 mm column, 0-100% MeCN in water) to afford the title compound (GB-2) (25.38 mg, 46 μmol, 80%, 98% Purity) as a white solid. m/z 538.5 (M+H)⁺ (ES+). ¹H NMR (400 MHz, DMSO) δ 8.42 (s, 1H), 8.15-8.10 (m, 1H), 8.07 (s, 1H), 7.91 (t, J=1.8 Hz, 1H), 7.80-7.74 (m, 2H), 7.64-7.53 (m, 3H), 7.41-7.36 (m, 1H), 4.61 (dd, J=7.7, 5.9 Hz, 2H), 4.25 (t, J=5.9 Hz, 2H), 3.88 (s, 2H), 3.20 (s, 3H), 3.09-2.99 (m, 1H), 2.77 (d, J=7.4 Hz, 2H). One exchangeable proton not observed.

### Example 254: N-((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)cyclobutanamine (GC-2)

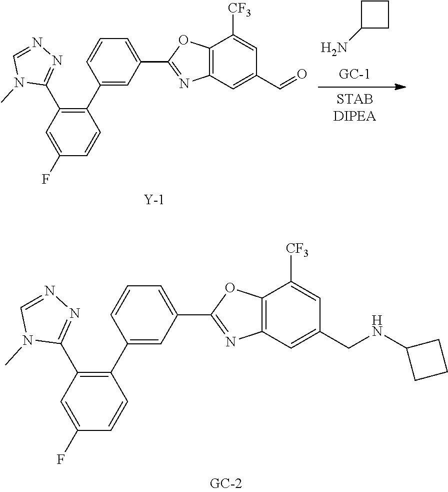

A solution of intermediate (Y-1) (30 mg, 90% Wt, 1 Eq, 58 μmol), DIPEA (22 mg, 30 μL, 3.0 Eq, 0.17 mmol) and cyclobutanamine (GC-2) (9.1 mg, 90% Wt, 2.0 Eq, 0.12 mmol) in CHCl₃ (3 mL) was stirred for 1 h at rt. NaBH(OAc)₃ (37 mg, 3.0 Eq, 0.17 mmol) was added to the reaction mixture and stirred for 16 h at rt. The reaction mixture was diluted with EtOAc (5 mL) and washed with a sat. aq sol. of NaHCO₃ (5 mL) and brine (5 mL). The organic layer was extracted, dried (MgSO₄), and concentrated in vacuo. The crude product was purified by preparative HPLC (Waters, Basic (0.1% ammonium bicarbonate), Basic, Waters X-Bridge Prep-C18, 5 μm, 30×100 mm column, 0-100% MeCN in water) to afford the title compound (GC-2) (26.51 mg, 48 μmol, 83%, 95% Purity) as a white solid. m/z 522.3 (M+H)⁺ (ES+). ¹H NMR (400 MHz, DMSO) δ 8.42 (s, 1H), 8.13 (dt, J=7.8, 1.4 Hz, 1H), 8.05 (s, 1H), 7.91 (t, J=1.8 Hz, 1H), 7.80-7.74 (m, 2H), 7.66-7.52 (m, 3H), 7.38 (dt, J=7.7, 1.5 Hz, 1H), 3.80 (s, 2H), 3.20 (s, 3H), 3.19-3.13 (m, 1H), 2.11-2.01 (m, 2H), 1.91 (s, 1H), 1.77-1.47 (m, 4H).

### Example 255: 2-Ethoxy-N-((2-(4′-fluoro-2′-(4-methyl-4H-1,2,4-triazol-3-yl)-[1,1′-biphenyl]-3-yl)-7-methoxybenzo[d]oxazol-5-yl)methyl)ethan-1-amine (GD-1)

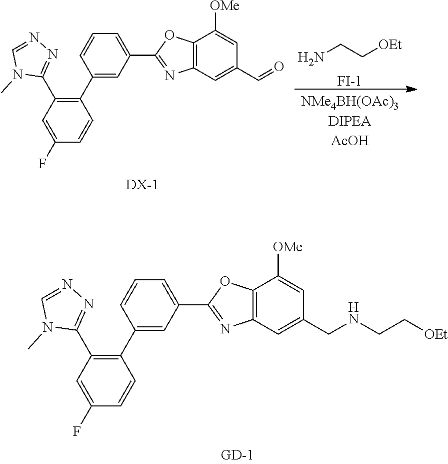

To one well of a 96-well plate were added 2-ethoxyethan-1-amine (FI-1) (9.4 mg, 1.5 Eq, 0.11 mmol) followed by a solution of intermediate (DX-1) (30 mg, 1 Eq, 70 μmol) in DMF (0.5 mL). DIPEA (18 mg, 24 μL, 2 Eq, 0.14 mmol) was added and the mixture shaken for 30 min. A solution of NMe₄BH(OAc)₃ (46 mg, 2.5 Eq, 0.18 mmol) in DMF (0.5 mL) and AcOH (11 mg, 10 μL, 2.5 Eq, 0.18 mmol) was added and the plate was shaken at rt for 3 d. A solution of NH₃/MeOH (200 μL) was added and the crude mixture directly purified by preparative HPLC (Waters, basic (0.3% ammonia), basic, Waters XBridge BEH C18 ODB, 5 μm, 30×100 mm column, MeCN/water) to afford impure product. The crude material was further purified by preparative HPLC (Waters, Acidic (0.1% formic acid), Acidic, X-Select CSH C18 ODB prep column, 130A, 5 μm, 30 mm×100 mm column, 10-100% MeCN in Water) to afford the title compound (GD-1) (5.63 mg, 0.011 mmol, 15.2%, 95% Purity) as a white solid. m/z 502.3 (M+H)⁺ (ES+). ¹H NMR (400 MHz, DMSO) δ 8.41 (s, 1H), 8.19 (s, 1H), 8.12-8.08 (m, 1H), 7.98-7.94 (m, 1H), 7.78 (dd, J=8.6, 5.7 Hz, 1H), 7.60 (td, J=8.5, 2.8 Hz, 1H), 7.56-7.48 (m, 2H), 7.33 (d, J=1.2 Hz, 1H), 7.31-7.24 (m, 1H), 7.09 (d, J=1.3 Hz, 1H), 4.01 (s, 3H), 3.85 (s, 2H), 3.50-3.39 (m, 3H), 3.13 (s, 3H), 2.69 (t, J=5.8 Hz, 2H), 1.11 (t, J=7.0 Hz, 3H). One exchangeable proton not observed.

### Example 256: N-((2-(4′-Fluoro-2′-(4-methyl-4H-1,2,4-triazol-3-yl)-[1,1′-biphenyl]-3-yl)-7-methoxybenzo[d]oxazol-5-yl)methyl)-2-isopropoxyethan-1-amine, formic Acid (GE-2)

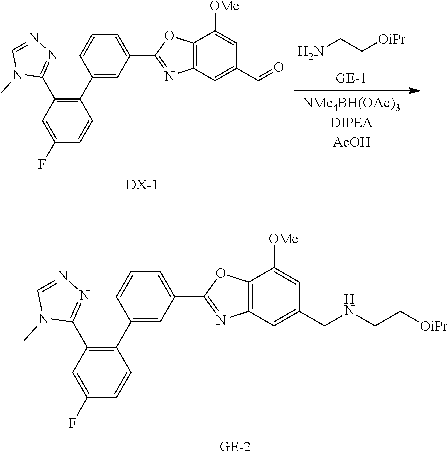

To one well of a 96-well plate were added 2-isopropoxyethan-1-amine (GE-1) (10.8 mg, 1.5 Eq, 0.11 mmol) followed by a solution of intermediate (DX-1) (30 mg, 1 Eq, 70 μmol) in DMF (0.5 mL). DIPEA (18 mg, 24 μL, 2 Eq, 0.14 mmol) was added and the mixture shaken for 30 min. A solution of NMe₄BH(OAc)₃ (46 mg, 2.5 Eq, 0.18 mmol) in DMF (0.5 mL) and AcOH (11 mg, 10 μL, 2.5 Eq, 0.18 mmol) was added and the plate was shaken at rt for 3 d. A solution of NH₃/MeOH (200 μL) was added and the crude mixture directly purified by preparative HPLC (Waters, basic (0.3% ammonia), basic, Waters XBridge BEH C18 ODB, 5 μm, 30×100 mm column, MeCN/water) to afford impure product. The crude material was further purified by preparative HPLC (Waters, Acidic (0.1% formic acid), Acidic, X-Select CSH C18 ODB prep column, 130A, 5 μm, 30 mm×100 mm column, 10-100% MeCN in Water) to afford the title compound (GE-2) (6.27 mg, 0.012 mmol, 16.5%, 95% Purity) as a white solid. m/z 516.3 (M+H)⁺ (ES+). ¹H NMR (400 MHz, DMSO) δ 8.18 (s, 1H), 8.11 (dt, J=8.0, 1.3 Hz, 1H), 7.96 (t, J=1.8 Hz, 1H), 7.78 (dd, J=8.7, 5.7 Hz, 1H), 7.60 (td, J=8.5, 2.8 Hz, 1H), 7.55-7.49 (m, 2H), 7.33 (d, J=1.3 Hz, 1H), 7.27 (dt, J=7.8, 1.5 Hz, 1H), 7.08 (d, J=1.3 Hz, 1H), 4.01 (s, 3H), 3.85 (s, 2H), 3.54 (p, J=6.1 Hz, 1H), 3.46 (t, J=5.8 Hz, 2H), 3.13 (s, 3H), 2.67 (t, J=5.7 Hz, 2H), 1.08 (d, J=6.1 Hz, 6H). NH not observed.

### Example 257: (S)-1-Cyclobutyl-N-((2-(4′-fluoro-2′-(4-methyl-4H-1,2,4-triazol-3-yl)-[1,1′-biphenyl]-3-yl)-7-methoxybenzo[d]oxazol-5-yl)methyl)ethan-1-amine (GF-2)

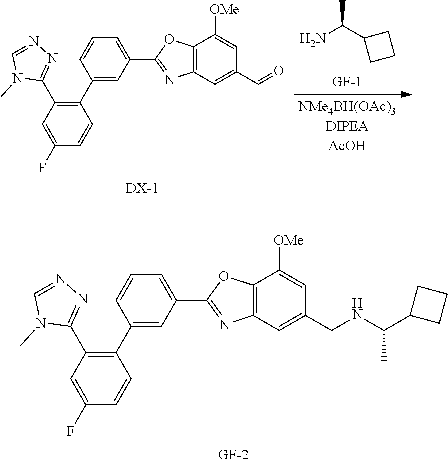

To one well of a 96-well plate were added (S)-1-cyclobutylethan-1-amine (GF-1) (10.4 mg, 1.5 Eq, 0.11 mmol) followed by a solution of intermediate (DX-1) (30 mg, 1 Eq, 70 μmol) in DMF (0.5 mL). DIPEA (18 mg, 24 μL, 2 Eq, 0.14 mmol) was added and the mixture shaken for 30 min. A solution of NMe₄BH(OAc)₃ (46 mg, 2.5 Eq, 0.18 mmol) in DMF (0.5 mL) and AcOH (11 mg, 10 μL, 2.5 Eq, 0.18 mmol) was added and the plate was shaken at rt for 3 d. A solution of NH₃/MeOH (200 μL) was added and the crude mixture directly purified by preparative HPLC (Waters, basic (0.3% ammonia), basic, Waters XBridge BEH C18 ODB, 5 μm, 30×100 mm column, MeCN/water) to afford the title compound (GF-2) (8.54 mg, 0.017 mmol, 23.8%, 98% Purity) as a white solid. m/z 511.2 (M+H)⁺ (ES+). ¹H NMR (400 MHz, DMSO) δ 8.41 (s, 1H), 8.10 (dt, J=7.8, 1.4 Hz, 1H), 7.96 (t, J=1.8 Hz, 1H), 7.78 (dd, J=8.6, 5.6 Hz, 1H), 7.60 (td, J=8.5, 2.8 Hz, 1H), 7.54-7.48 (m, 2H), 7.32 (d, J=1.2 Hz, 1H), 7.26 (dt, J=8.0, 1.3 Hz, 1H), 7.09 (d, J=1.3 Hz, 1H), 4.00 (s, 3H), 3.85 (d, J=14.0 Hz, 1H), 3.74 (d, J=14.0 Hz, 1H), 3.13 (s, 3H), 2.46-2.40 (m, 1H), 2.18 (q, J=8.0 Hz, 1H), 1.99 (dd, J=8.2, 4.0 Hz, 1H), 1.90-1.83 (m, 1H), 1.81-1.62 (m, 4H), 0.89 (d, J=6.1 Hz, 3H). NH not observed.

### Example 258: 2-Cyclobutyl-N-((2-(4′-fluoro-2′-(4-methyl-4H-1,2,4-triazol-3-yl)-[1,1′-biphenyl]-3-yl)-7-methoxybenzo[d]oxazol-5-yl)methyl)ethan-1-amine (GG-2)

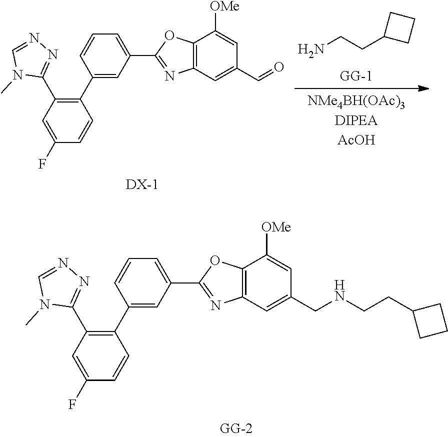

To one well of a 96-well plate were added 2-cyclobutylethan-1-amine (GG-1) (10.4 mg, 1.5 Eq, 0.11 mmol) followed by a solution of intermediate (DX-1) (30 mg, 1 Eq, 70 μmol) in DMF (0.5 mL). DIPEA (18 mg, 24 μL, 2 Eq, 0.14 mmol) was added and the mixture shaken for 30 min. A solution of NMe₄BH(OAc)₃ (46 mg, 2.5 Eq, 0.18 mmol) in DMF (0.5 mL) and AcOH (11 mg, 10 μL, 2.5 Eq, 0.18 mmol) was added and the plate was shaken at rt for 3 d. A solution of NH₃/MeOH (200 μL) was added and the crude mixture directly purified by preparative HPLC (Waters, basic (0.3% ammonia), basic, Waters XBridge BEH C18 ODB, 5 μm, 30×100 mm column, MeCN/water) to afford the title compound (GG-2) (2.22 mg, 0.0043 mmol, 6%, 97% Purity) as a white solid. m/z 511.6 (M+H)⁺ (ES+). ¹H NMR (400 MHz, DMSO) δ 8.41 (s, 1H), 8.10 (dt, J=7.9, 1.4 Hz, 1H), 7.96 (t, J=1.8 Hz, 1H), 7.81-7.73 (m, 1H), 7.59 (td, J=8.6, 2.8 Hz, 1H), 7.56-7.49 (m, 2H), 7.30 (d, J=1.2 Hz, 1H), 7.26 (dt, J=7.8, 1.5 Hz, 1H), 7.06 (s, 1H), 4.00 (s, 3H), 3.76 (s, 2H), 3.13 (s, 3H), 2.40 (t, J=7.2 Hz, 2H), 2.36-2.27 (m, 1H), 2.03-1.94 (m, 2H), 1.85-1.73 (m, 2H), 1.62-1.51 (m, 4H). One exchangeable proton not observed.

### Example 259: N-((2-(4′-Fluoro-2′-(4-methyl-4H-1,2,4-triazol-3-yl)-[1,1′-biphenyl]-3-yl)-7-methoxybenzo[d]oxazol-5-yl)methyl)cyclobutanamine (GH-1)

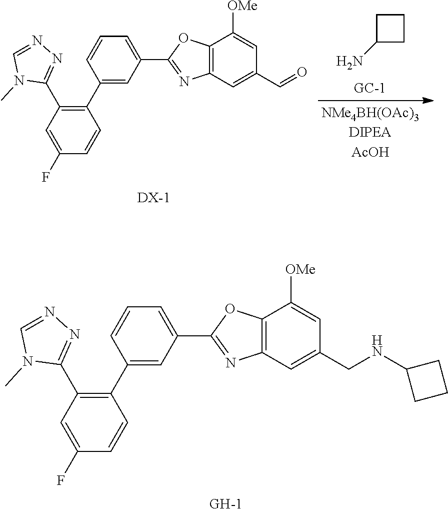

To one well of a 96-well plate were added cyclobutanamine (GC-1) (7.5 mg, 1.5 Eq, 0.11 mmol) followed by a solution of intermediate (DX-1) (30 mg, 1 Eq, 70 μmol) in DMF (0.5 mL). DIPEA (18 mg, 24 μL, 2 Eq, 0.14 mmol) was added and the mixture shaken for 30 min. A solution of NMe₄BH(OAc)₃ (46 mg, 2.5 Eq, 0.18 mmol) in DMF (0.5 mL) and AcOH (11 mg, 10 μL, 2.5 Eq, 0.18 mmol) was added and the plate was shaken at rt for 3 d. A solution of NH₃/MeOH (200 L) was added and the crude mixture directly purified by preparative HPLC (Waters, basic (0.3% ammonia), basic, Waters XBridge BEH C18 ODB, 5 μm, 30×100 mm column, MeCN/water) to afford the title compound (GH-1) (10.66 mg, 0.022 mmol, 31.5%, 99% Purity) as a white solid. m/z 483.55 (M+H)⁺ (ES+). ¹H NMR (400 MHz, DMSO) δ 8.41 (s, 1H), 8.10 (dt, J=7.9, 1.3 Hz, 1H), 7.96 (t, J=1.8 Hz, 1H), 7.84-7.76 (m, 1H), 7.61-7.56 (m, 1H), 7.55-7.46 (m, 2H), 7.30-7.22 (m, 2H), 7.05 (d, J=1.3 Hz, 1H), 4.00 (s, 3H), 3.70 (s, 2H), 3.13 (s, 3H), 2.11-1.99 (m, 2H), 1.75-1.44 (m, 4H). 2H not observed.

### Example 260: (1S,2R)-2-(((2-(4′-Fluoro-2′-(4-methyl-4H-1,2,4-triazol-3-yl)-[1,1′-biphenyl]-3-yl)-7-methoxybenzo[d]oxazol-5-yl)methyl)amino)-1-methylcyclopentan-1-ol, Formic Acid (GI-2)

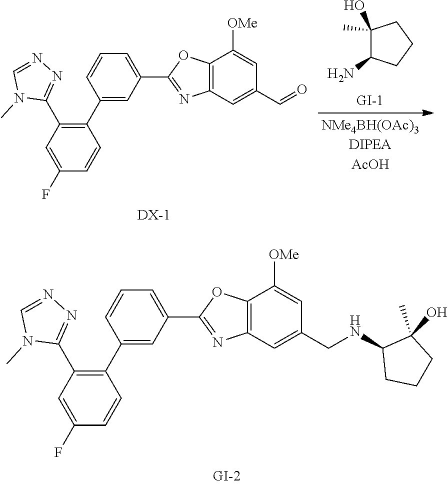

To one well of a 96-well plate were added (1S,2R)-2-amino-1-methylcyclopentan-1-ol (GI-1) (12.1 mg, 1.5 Eq, 0.11 mmol) followed by a solution of intermediate (DX-1) (30 mg, 1 Eq, 70 μmol) in DMF (0.5 mL). DIPEA (18 mg, 24 μL, 2 Eq, 0.14 mmol) was added and the mixture shaken for 30 min. A solution of NMe₄BH(OAc)₃ (46 mg, 2.5 Eq, 0.18 mmol) in DMF (0.5 mL) and AcOH (11 mg, 10 μL, 2.5 Eq, 0.18 mmol) was added and the plate was shaken at rt for 3 d. A solution of NH₃/MeOH (200 μL) was added and the crude mixture directly purified by preparative HPLC (Waters, basic (0.3% ammonia), basic, Waters XBridge BEH C18 ODB, 5 μm, 30×100 mm column, MeCN/water) to afford impure product. The crude material was further purified by preparative HPLC (Waters, Acidic (0.1% formic acid), Acidic, X-Select CSH C18 ODB prep column, 130A, 5 μm, 30 mm×100 mm column, 10-100% MeCN in Water) to afford the title compound (GI-2) (2.51 mg, 0.0048 mmol, 6.8%, 95% Purity) as a white solid. m/z 527.6 (M+H)⁺ (ES+). ¹H NMR (400 MHz, DMSO) δ 8.41 (s, 1H), 8.18 (s, 1H), 8.14-8.09 (m, 1H), 7.96 (t, J=1.8 Hz, 1H), 7.78 (dd, J=8.6, 5.6 Hz, 1H), 7.60 (td, J=8.6, 2.8 Hz, 1H), 7.54-7.49 (m, 2H), 7.34 (s, 1H), 7.27 (dt, J=7.8, 1.5 Hz, 1H), 7.09 (d, J=1.3 Hz, 1H), 4.29 (s, 1H), 4.01 (s, 3H), 3.93-3.76 (m, 2H), 3.13 (s, 3H), 2.71 (t, J=7.4 Hz, 1H), 1.89 (d, J=11.2 Hz, 1H), 1.55 (d, J=3.7 Hz, 5H), 1.16 (s, 3H). One exchangeable proton not observed and one additional proton from formic acid.

### Example 261: (1R,2R)-2-(((2-(4′-fluoro-2′-(4-methyl-4H-1,2,4-triazol-3-yl)-[1,1′-biphenyl]-3-yl)-7-methoxybenzo[d]oxazol-5-yl)methyl)amino)-1-methylcyclopentan-1-ol, Formic Acid (GJ-2)

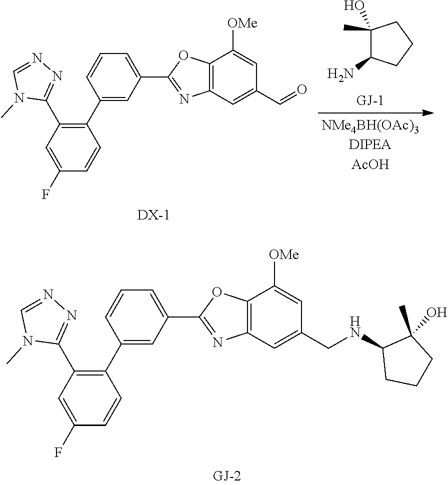

To one well of a 96-well plate were added (1R,2R)-2-amino-1-methylcyclopentan-1-ol (GJ-1) (12.1 mg, 1.5 Eq, 0.11 mmol) followed by a solution of intermediate (DX-1) (30 mg, 1 Eq, 70 μmol) in DMF (0.5 mL). DIPEA (18 mg, 24 μL, 2 Eq, 0.14 mmol) was added and the mixture shaken for 30 min. A solution of NMe₄BH(OAc)₃ (46 mg, 2.5 Eq, 0.18 mmol) in DMF (0.5 mL) and AcOH (11 mg, 10 μL, 2.5 Eq, 0.18 mmol) was added and the plate was shaken at rt for 3 d. A solution of NH₃/MeOH (200 μL) was added and the crude mixture directly purified by preparative HPLC (Waters, basic (0.3% ammonia), basic, Waters XBridge BEH C18 ODB, 5 μm, 30×100 mm column, MeCN/water) to afford impure product. The crude material was further purified by preparative HPLC (Waters, Acidic (0.1% formic acid), Acidic, X-Select CSH C18 ODB prep column, 130A, 5 μm, 30 mm×100 mm column, 10-100% MeCN in Water) to afford the title compound (GJ-2) (2.75 mg, 0.0052 mmol, 7.1%, 95% Purity) as a white solid. m/z 527.6 (M+H)⁺ (ES+). ¹NMR (400 MHz, DMSO) δ 8.41 (s, 1H), 8.18 (s, 1H), 8.10 (dt, J=7.8, 1.4 Hz, 1H), 7.96 (t, J=1.8 Hz, 1H), 7.78 (dd, J=8.7, 5.7 Hz, 1H), 7.60 (td, J=8.5, 2.8 Hz, 1H), 7.55-7.49 (m, 2H), 7.34 (d, J=1.2 Hz, 1H), 7.27 (dt, J=8.0, 1.3 Hz, 1H), 7.09 (d, J=1.3 Hz, 1H), 4.29 (s, 1H), 4.01 (s, 3H), 3.92-3.77 (m, 2H), 3.13 (s, 3H), 2.71 (t, J=7.4 Hz, 1H), 1.92-1.83 (m, 1H), 1.59-1.47 (m, 5H), 1.16 (s, 3H). One exchangeable proton not observed, and an additional proton associated with formic acid.

### Example 262: (1S,2R)-2-(((2-(4′-Fluoro-2′-(4-methyl-4H-1,2,4-triazol-3-yl)-[1,1′-biphenyl]-3-yl)-[1,2,4]triazolo[1,5-a]pyridin-6-yl)methyl)amino)cyclopentan-1-ol, Formic Acid (GK-5)

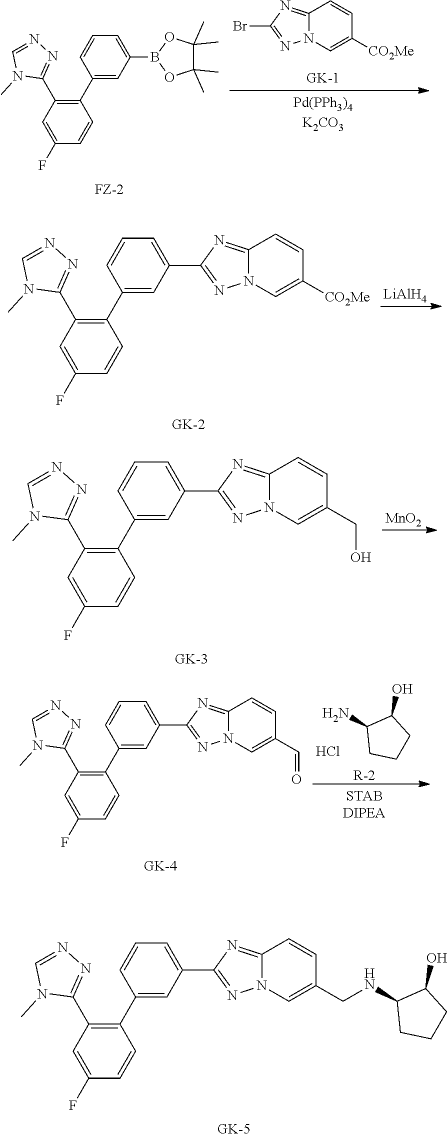

Step 1: Methyl 2-(4′-fluoro-2′-(4-methyl-4H-1,2,4-triazol-3-yl)-[1,1′-biphenyl]-3-yl)-[1,2,4]triazolo[1,5-a]pyridine-6-carboxylate (GK-2). K₂CO₃ (47 mg, 3 Eq, 0.34 mmol) in water (0.3 mL) was added to a mixture of Pd(PPh₃)₄ (13 mg, 0.1 Eq, 11 μmol), intermediate (FZ-2) (45 mg, 95% Wt, 1 Eq, 0.11 mmol) and methyl 2-bromo-[1,2,4]triazolo[1,5-a]pyridine-6-carboxylate (GK-1) (29 mg, 100% Wt, 1 Eq, 0.11 mmol) in toulene (1.2 mL). The mixture was then stirred at 100° C. (microwave) for 45 min. The reaction mixture partitioned between DCM (20 mL) and water (20 mL). The organic layer was separated and adsorbed on to silica-gel. The crude product was purified by chromatography on silica gel (12 g cartridge, 0-10% MeOH/DCM) to afford the sub-title compound (GK-2) (32 mg, 66 μmol, 58%, 88% Purity) as a pale brown glass. m/z 429.1 (M+H)⁺ (ES+).

Step 2: (2-(4′-Fluoro-2′-(4-methyl-4H-1,2,4-triazol-3-yl)-[1,1′-biphenyl]-3-yl)-[1,2,4]triazolo[1,5-a]pyridin-6-yl)methanol (GK-3). A solution of LiAlH₄ in THF (6.2 mg, 0.16 mL, 1 molar, 1.5 Eq, 0.16 mmol) was added to a stirred suspension of the product from step 1 above (GK-2) (54 mg, 86% Wt, 1 Eq, 0.11 mmol) in THF (3 mL) at 0° C. The reaction mixture was stirred at 0° C. for 60 min. The reaction mixture was diluted with THF (5 ml) before being vigorously stirred with sodium sulfate decahydrate (c. 600 mg) added portion-wise at 0° C. The reaction mixture was partitioned between EtOAc (20 mL) and water (20 mL). The biphasic mixture was separated, and the aqueous layer re-extracted with EtOAc (20 mL). The organic extracts were combined, dried (MgSO₄) and concentrated in vacuo to afford the sub-title compound (GK-3) (33 mg, 60 μmol, 56%, 73% Purity) as a pale yellow glass. m/z 401.3 (M+H)⁺ (ES+).

Step 3: 2-(4′-Fluoro-2′-(4-methyl-4H-1,2,4-triazol-3-yl)-[1,1′-biphenyl]-3-yl)-[1,2,4]triazolo[1,5-a]pyridine-6-carbaldehyde (GK-4). The product from step 2 above (GK-3) (33 mg, 73% Wt, 1 Eq, 60 μmol) and MnO₂ (0.21 g, 42 μL, 40 Eq, 2.4 mmol) in CHCl₃ (3 mL) was stirred at rt over the weekend. The reaction mixture was filtered through a small Celite plug (chased with 10 mL DCM). The filtrate was evaporated to dryness to afford the sub-title compound (GK-4) (20 mg, 43 μmol, 72%, 86% Purity) as a pale-yellow glass. m/z 399.6 (M+H)⁺ (ES+).

Step 4: (1S,2R)-2-(((2-(4′-Fluoro-2′-(4-methyl-4H-1,2,4-triazol-3-yl)-[1,1′-biphenyl]-3-yl)-[1,2,4]triazolo[1,5-a]pyridin-6-yl)methyl)amino)cyclopentan-1-ol, Formic Acid (GK-5). NaBH(OAc)₃ (27 mg, 3.0 Eq, 0.13 mmol) was added to a stirred mixture of the product from step 3 above (GK-4) (20 mg, 86% Wt, 1 Eq, 43 μmol), (1S,2R)-2-aminocyclopentan-1-ol, HCl (R-2) (12 mg, 2.0 Eq, 86 μmol) and DIPEA (17 mg, 23 μL, 3.0 Eq, 0.13 mmol) in CHCl₃ (2 mL). The reaction mixture was stirred at rt overnight. The reaction mixture was diluted with DCM (4 mL) and washed with sat. aq. NaHCO₃ (10 mL). The organic phase was separated and evaporated to afford the crude product. The crude product was purified by preparative HPLC (Waters, acidic (0.1% formic acid), acidic, Waters X-Select CSH C18 ODB prep column, 130A, 5 μm, 30 mm×100 mm column, 10-100% MeCN in water) to afford the title compound (GK-5) (3 mg, 6 μmol, 10%, 99% Purity) as a colourless foam. m/z 484.0 (M+H)⁺ (ES+). ¹H NMR (400 MHz, MeOD) δ 9.00 (s, 1H), 8.48 (s, 1H), 8.38 (s, 1H), 8.22 (dt, J=7.8, 1.4 Hz, 1H), 8.12 (t, J=1.8 Hz, 1H), 7.87-7.77 (m, 3H), 7.60-7.50 (m, 2H), 7.48 (dd, J=8.8, 2.8 Hz, 1H), 7.36 (ddd, J=7.7, 2.0, 1.2 Hz, 1H), 4.45-4.39 (m, 1H), 4.39-4.31 (m, 2H), 3.56-3.46 (m, 1H), 3.18 (s, 3H), 2.21-2.09 (m, 1H), 2.09-1.78 (m, 4H), 1.78-1.66 (m, 1H). Formic acid salt proton @8.48 ppm. NH and OH not observed.

### Example 263: (1S,2R)-2-(((2-(4′-Fluoro-2′-(4-methyl-4H-1,2,4-triazol-3-yl)-[1,1′-biphenyl]-3-yl)imidazo[1,2-a]pyridin-6-yl)methyl)amino)cyclopentan-1-ol (GL-2)

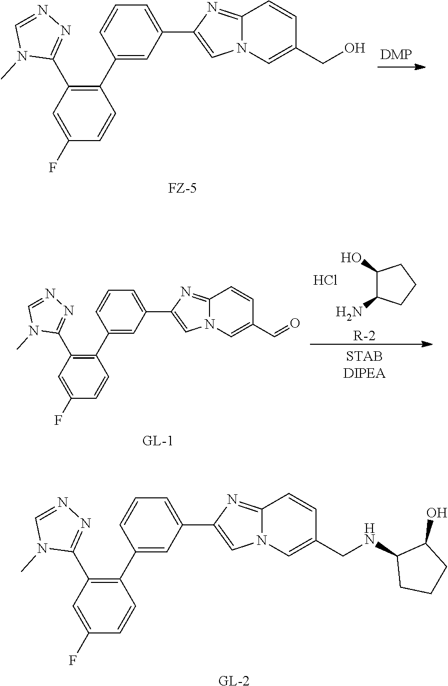

Step 1: 2-(4′-Fluoro-2′-(4-methyl-4H-1,2,4-triazol-3-yl)-[1,1′-biphenyl]-3-yl)imidazo[1,2-a]pyridine-6-carbaldehyde (GL-1). To a stirred solution of compound (FZ-5) (96.4 mg, 50% Wt, 1 Eq, 121 μmol) in dry DCM (3.0 mL) at rt was added Dess-Martin periodinane (120.1 mg, 2.35 Eq, 283.2 μmol). The reaction mixture was stirred at rt for 1 h. Additional Dess-Martin periodinane (100.7 mg, 1.97 Eq, 237.4 μmol) was added and the resulting mixture was stirred at rt overnight. Further Dess-Martin periodinane (106.5 mg, 2.08 Eq, 251.1 μmol) was added and the reaction mixture was stirred at rt overnight. The volatiles were removed in vacuo. The crude product was purified by chromatography on silica gel (12 g cartridge, 0-10% MeOH/DCM) to afford the sub-title compound (GL-1) (31.9 mg, 63 μmol, 52%, 78% Purity) as a yellow solid, alongside recovered (FZ-5) (21.4 mg, 40 μmol, 33%, 75% Purity) as a yellow solid. m/z 398.2 (M+H)⁺ (ES+). ¹H NMR (400 MHz, MeOD) δ 9.95 (s, 1H), 8.36 (s, 1H), 8.25 (s, 1H), 8.20-8.12 (m, 1H), 8.04-7.92 (m, 3H), 7.89 (dd, J=8.3, 1.0 Hz, 1H), 7.81-7.70 (m, 2H), 7.53 (td, J=8.4, 2.8 Hz, 1H), 7.47-7.42 (m, 1H), 7.21-7.17 (m, 1H), 3.12 (s, 3H).

Step 2: (1S,2R)-2-(((2-(4′-fluoro-2′-(4-methyl-4H-1,2,4-triazol-3-yl)-[1,1′-biphenyl]-3-yl)imidazo[1,2-a]pyridin-6-yl)methyl)amino)cyclopentan-1-ol (GL-2). A solution of the product from step 1 above (GL-1) (31.9 mg, 1 Eq, 80.3 μmol), (1S,2R)-2-aminocyclopentan-1-ol, HCl (R-2) (22.6 mg, 2.05 Eq, 164 μmol) and DIPEA (52 mg, 70 μL, 5.0 Eq, 0.40 mmol) in CHCl₃ (3.0 mL) was stirred 1 h at rt. NaBH(OAc)₃ (55.3 mg, 97% Wt, 3.15 Eq, 253 μmol) was added and the reaction mixture was stirred at rt overnight. Additional NaBH(OAc)₃ (55.4 mg, 97% Wt, 3.16 Eq, 254 μmol) was added and the resulting mixture was stirred at rt for 3 h. The reaction mixture was diluted with EtOAc (2 mL), washed with sat. NaHCO₃ (2 mL) and brine (2 mL), dried (Na₂SO₄), filtered and concentrated in vacuo. The crude product was purified by chromatography on silica gel (4 g cartridge, 0-10% MeOH/DCM) to afford the title compound (GL-2) (2.0 mg, 3.7 μmol, 4.6%, 90% Purity) as a pale yellow solid. m/z 483.2 (M+H)⁺ (ES+). ¹H NMR (400 MHz, MeOD) δ 8.48 (s, 1H), 8.36 (s, 1H), 8.06 (s, 1H), 7.90 (d, J=7.9 Hz, 1H), 7.76 (dd, J=8.6, 5.5 Hz, 1H), 7.72 (t, J=1.8 Hz, 1H), 7.58-7.48 (m, 2H), 7.45-7.38 (m, 3H), 7.19-7.15 (m, 1H), 4.26-4.21 (m, 1H), 4.02 (d, J=13.5 Hz, 1H), 3.95 (d, J=13.3 Hz, 1H), 3.14-3.12 (m, 1H), 3.11 (s, 3H), 1.93-1.82 (m, 2H), 1.82-1.72 (m, 1H), 1.67-1.55 (m, 3H). 2× Exchangeable protons not observed.

### Example 264: N-((2-(4′-Fluoro-2′-(4-methyl-4H-1,2,4-triazol-3-yl)-[1,1′-biphenyl]-3-yl)-7-methoxybenzo[d]oxazol-5-yl)methyl)-2-methoxy-N-methylethan-1-amine (GM-2)

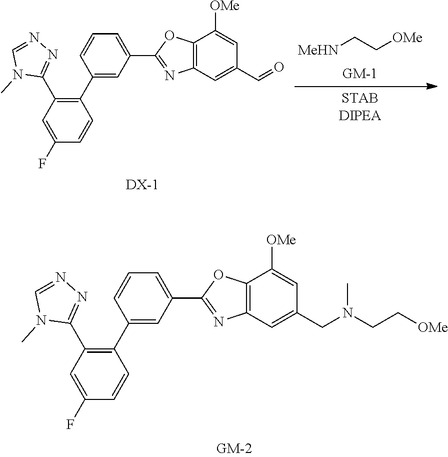

NaBH(OAc)₃ (44 mg, 3.0 Eq, 0.21 mmol) was added to a stirred mixture of (DX-1) (30 mg, 98% Wt, 1 Eq, 69 μmol), 2-methoxy-N-methylethan-1-amine (GM-1) (9.2 mg, 1.5 Eq, 0.10 mmol) and DIPEA (27 mg, 37 μL, 3.0 Eq, 0.21 mmol) in CHCl₃ (2 mL). The reaction mixture was stirred at rt overnight. The reaction mixture was diluted with DCM (4 mL) and washed with sat. aq. NaHCO₃ (10 mL). The organic phase was separated and evaporated to afford the crude product. The crude product was purified by chromatography on silica gel (4 g cartridge, 0-10% MeOH/DCM) to afford the title compound (GM-2) (26 mg, 50 μmol, 73%, 97% Purity) as a white solid. m/z 502.3 (M+H)⁺ (ES+). ¹HNMR (400 MHz, DMSO) δ 8.41 (s, 1H), 8.10 (dt, J=8.0, 1.3 Hz, 1H), 7.96 (t, J=1.8 Hz, 1H), 7.78 (dd, J=8.6, 5.7 Hz, 1H), 7.60 (td, J=8.6, 2.9 Hz, 1H), 7.56-7.48 (m, 2H), 7.29-7.25 (m, 2H), 7.03 (d, J=1.3 Hz, 1H), 4.00 (s, 3H), 3.61 (s, 2H), 3.47 (t, J=5.9 Hz, 2H), 3.24 (s, 3H), 3.13 (s, 3H), 2.55 (t, J=5.9 Hz, 2H), 2.21 (s, 3H).

### Example 265: (2-(4′-Fluoro-2′-(4-methyl-4H-1,2,4-triazol-3-yl)-[1,1′-biphenyl]-3-yl)imidazo[1,2-a]pyridin-7-yl)methanol (GN-5)

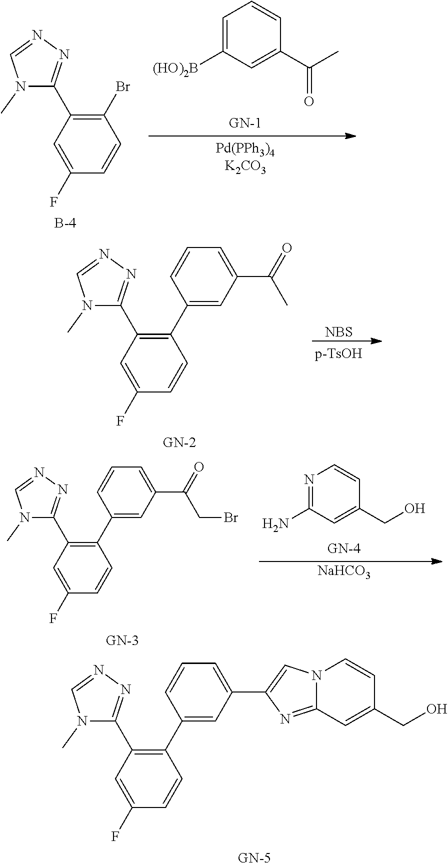

Step 1: 1-(4′-Fluoro-2′-(4-methyl-4H-1,2,4-triazol-3-yl)-[1,1′-biphenyl]-3-yl)ethan-1-one (GN-2). A solution of intermediate (B-4) (523.1 mg, 99% Wt, 1 Eq, 2.022 mmol), (3-acetylphenyl)boronic acid (GN-1) (379.5 mg, 1.144 Eq, 2.314 mmol) and K₂CO₃ (847.3 mg, 3.032 Eq, 6.131 mmol) in dioxane (10 mL) and water (2.5 mL) was degassed for 10 min. Pd(PPh₃)₄ (60.4 mg, 0.0258 Eq, 52.3 μmol) was added and the resulting mixture was heated at 100° C. under a nitrogen atmosphere for 7 h. Additional Pd(PPh₃)₄ (64.3 mg, 0.0275 Eq, 55.6 μmol) was added and the reaction mixture was heated at 100° C. overnight. The reaction mixture was cooled to rt. The reaction mixture was diluted with brine (10 mL), extracted with EtOAc (3×10 mL). The combined organic layers were collected, dried (Na₂SO₄), filtered and concentrated in vacuo. The crude product was purified by chromatography on silica gel (24 g cartridge, 0-10% MeOH/DCM) to afford the sub-title compound (GN-2) (558.1 mg, 1.7 mmol, 84%, 90% Purity) as a brown oil. m/z 296.4 (M+H)⁺ (ES+). ¹H NMR (400 MHz, DMSO) δ 8.42 (s, 1H), 7.86 (dt, J=7.8, 1.3 Hz, 1H), 7.72 (dd, J=8.6, 5.7 Hz, 1H), 7.66 (t, J=1.8 Hz, 1H), 7.62-7.55 (m, 1H), 7.51 (dd, J=9.1, 2.7 Hz, 1H), 7.46 (t, J=7.7 Hz, 1H), 7.37 (dt, J=7.8, 1.4 Hz, 1H), 3.08 (s, 3H), 2.48 (s, 3H).

Step 2: 2-Bromo-1-(4′-fluoro-2′-(4-methyl-4H-1,2,4-triazol-3-yl)-[1,1′-biphenyl]-3-yl)ethan-1-one (GN-3). To a stirred solution of the product from step 1 above (GN-2) (199.9 mg, 90% Wt, 1 Eq, 609.2 μmol) in MeCN (6.0 mL) was added p-TsOH-H₂O (129.2 mg, 1.115 Eq, 679.2 μmol) and NBS (113.7 mg, 1.049 Eq, 638.8 μmol). The reaction mixture was heated at 80° C. overnight. Additional p-TsOH-H₂O (29.1 mg, 0.251 Eq, 153 μmol) and NBS (26.7 mg, 0.246 Eq, 150 μmol) were added. The reaction mixture was heated at 80° C. for 2 h. Further p-TsOH-H₂O (23.7 mg, 0.205 Eq, 125 μmol) and NBS (21.7 mg, 0.200 Eq, 122 μmol) were added. The reaction mixture was heated at 80° C. for 1 h. The reaction was cooled to rt and the volatiles were removed in vacuo. The residue was redissolved in DCM (5 mL) and washed with H₂O (5 mL). The aqueous layer was extracted with DCM (3×5 mL). The combined organic layers was collected, dried (Na₂SO₄), filtered and concentrated in vacuo to afford the sub-title compound (GN-3) as a yellow solid, which was used directly in the next step without further purification or analysis.

Step 3: (2-(4′-fluoro-2′-(4-methyl-4H-1,2,4-triazol-3-yl)-[1,1′-biphenyl]-3-yl)imidazo[1,2-a]pyridin-7-yl)methanol (GN-5). To a stirred solution of the product from step 2 above (GN-3) (228 mg, 1 Eq, 609 μmol) and (2-aminopyridin-4-yl)methanol (GN-4) (80.3 mg, 1.06 Eq, 647 μmol) in abs. EtOH (3.0 mL) at rt was added NaHCO₃ (160.3 mg, 3.13 Eq, 1.908 mmol). The reaction mixture was heated at 50° C. overnight. The reaction mixture was cooled to rt and the volatiles were removed in vacuo. The crude product was purified by chromatography on silica gel (12 g cartridge, 0-10% MeOH/DCM) to afford product with ˜70% purity. The crude product was purified by preparative HPLC (Waters, Basic (0.3% ammonia), Basic, Waters X-Bridge BEH C18 ODB, 5 μm, 30×100 mm column 15-100% MeCN in water) to afford the title compound (GN-5) (15.0 mg, 37 μmol, 6.1%, 99% Purity) as a white solid. m/z 400.5 (M+H)⁺ (ES+). ¹H NMR (400 MHz, MeOD) δ 8.40-8.34 (m, 2H), 8.03 (d, J=0.7 Hz, 1H), 7.89 (dt, J=8.0, 1.3 Hz, 1H), 7.80-7.72 (m, 2H), 7.55-7.49 (m, 2H), 7.45-7.38 (m, 2H), 7.13 (ddd, J=7.7, 1.9, 1.1 Hz, 1H), 6.91 (dd, J=7.0, 1.6 Hz, 1H), 4.68 (d, J=1.2 Hz, 2H), 3.10 (s, 3H). 1× Exchangeable OH proton not observed.

### Example 266: (1S,2R)-2-(((2-(4′-Fluoro-2′-(4-methyl-4H-1,2,4-triazol-3-yl)-[1,1′-biphenyl]-3-yl)imidazo[1,2-a]pyridin-7-yl)methyl)amino)cyclopentan-1-ol (GO-2)

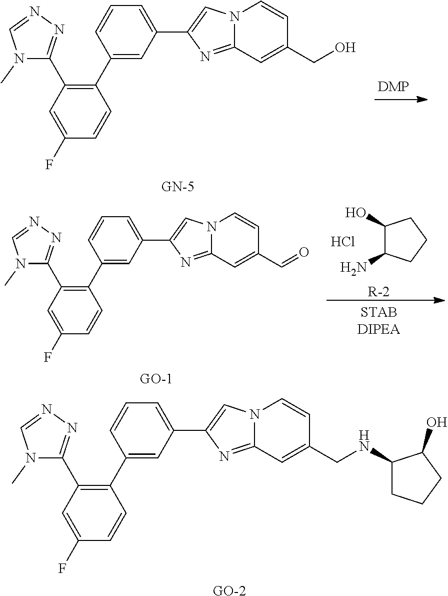

Step 1: 2-(4′-Fluoro-2′-(4-methyl-4H-1,2,4-triazol-3-yl)-[1,1′-biphenyl]-3-yl)imidazo[1,2-a]pyridine-7-carbaldehyde (GO-1). To a stirred solution of compound (GN-5) (13.1 mg, 1 Eq, 32.8 μmol) in dry DCM (3.0 mL) at rt was added Dess-Martin periodinane (30.5 mg, 2.19 Eq, 71.9 μmol). The reaction mixture was stirred at rt for 1 h. The reaction mixture was diluted with DCM (5 mL), washed with sat. NaHCO₃ (5 mL) and brine (5 mL). The organic layer was dried (Na₂SO₄), filtered and concentrated in vacuo. The crude product was purified by chromatography on silica gel (4 g cartridge, 0-10% MeOH/DCM) to afford the sub-title compound (GO-1) (12.4 mg, 22 μmol, 67%, 70% Purity) as a yellow solid. m/z 398.1 (M+H)⁺ (ES+). ¹H NMR (400 MHz, MeOD) δ 10.01 (s, 1H), 8.54 (d, J=7.0 Hz, 1H), 8.36 (s, 1H), 8.30 (s, 1H), 8.18-8.12 (m, 1H), 7.98-7.94 (m, 1H), 7.81 (t, J=1.8 Hz, 1H), 7.80-7.71 (m, 1H), 7.56-7.48 (m, 1H), 7.48-7.40 (m, 2H), 7.40-7.34 (m, 1H), 7.19 (dt, J=7.8, 1.4 Hz, 1H), 3.12 (s, 3H).

Step 2: (1S,2R)-2-(((2-(4′-Fluoro-2′-(4-methyl-4H-1,2,4-triazol-3-yl)-[1,1′-biphenyl]-3-yl)imidazo[1,2-a]pyridin-7-yl)methyl)amino)cyclopentan-1-ol (GO-2). A solution of the product from step 1 above (GO-1) (12.4 mg, 70% Wt, 1 Eq, 21.8 μmol), (1S,2R)-2-aminocyclopentan-1-ol, HCl (R-2) (7.9 mg, 2.6 Eq, 57 μmol) and DIPEA (22 mg, 30 μL, 7.9 Eq, 0.17 mmol) in CHCl₃ (2.0 mL) was stirred at rt for 1 h. NaBH(OAc)₃ (18.4 mg, 97% Wt, 3.86 Eq, 84.2 μmol) was added and the reaction mixture was stirred at rt overnight. NaBH(OAc)₃ (20.1 mg, 4.34 Eq, 94.8 μmol) was added and the resulting mixture was stirred at rt for 3 h. The reaction mixture was diluted with EtOAc (2 mL), washed with sat. NaHCO₃ (2 mL) and brine (2 mL), dried (Na₂SO₄), filtered and concentrated in vacuo. The crude product was purified by chromatography on silica gel (4 g cartridge, 0-20% MeOH/DCM) to afford the title compound (GO-2) (3.9 mg, 8.0 μmol, 37%, 99% Purity) as a pale-yellow solid. m/z 483.2 (M+H)⁺ (ES+). ¹H NMR (400 MHz, MeOD) δ 8.39 (dd, J=6.9, 0.9 Hz, 1H), 8.36 (s, 1H), 8.03 (s, 1H), 7.89 (dt, J=8.0, 1.3 Hz, 1H), 7.79-7.72 (m, 2H), 7.55-7.48 (m, 2H), 7.45-7.37 (m, 2H), 7.13 (ddd, J=7.7, 1.9, 1.1 Hz, 1H), 6.99 (dd, J=7.0, 1.6 Hz, 1H), 4.19-4.14 (m, 1H), 3.95 (d, J=13.9 Hz, 1H), 3.88 (d, J=13.9 Hz, 1H), 3.10 (s, 3H), 3.03-2.95 (m, 1H), 1.98-1.91 (m, 1H), 1.87-1.77 (m, 2H), 1.77-1.69 (m, 1H), 1.62-1.50 (m, 2H). 2× exchangeable protons not observed.

### Example 267: (1S,3R)-3-(((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)amino)-2,2-dimethylcyclobutan-1-ol, Formic Acid (GP-2)

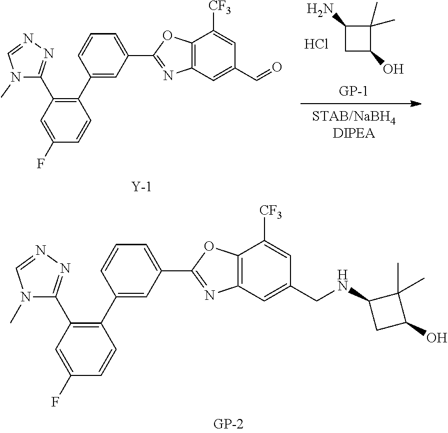

NaBH(OAc)₃ (37 mg, 3.0 Eq, 0.17 mmol) was added to a stirred mixture of intermediate (Y-1) (30 mg, 90% Wt, 1 Eq, 58 μmol), cis-3-Amino-2,2-dimethylcyclobutanol, HCl (GP-2) (18 mg, 2.0 Eq, 0.12 mmol) and DIPEA (22 mg, 31 μL, 3.0 Eq, 0.17 mmol) in CHCl₃ (3 mL). The reaction mixture was stirred at rt overnight. NaBH₄ (22 mg, 10.0 Eq, 0.58 mmol) was added to the reaction mixture and stirring continued at rt for an additional 4 hours. The reaction mixture was allowed to stand at rt over the weekend. The reaction mixture was diluted with DCM (4 mL) and washed with sat. aq. NaHCO₃ (10 mL). The organic phase was separated and evaporated to afford the crude product. The crude product was purified by preparative HPLC (Waters, acidic (0.1% formic acid), acidic, Waters X-Select CSH C18 ODB prep column, 130A, 5 μm, 30 mm×100 mm column, 15-100% MeCN in water) to afford the title compound (GP-2) (13 mg, 20 μmol, 35%, 96% Purity) as a white foam. m/z 566.4 (M+H)⁺ (ES+). ¹H NMR (400 MHz, DMSO) δ 8.42 (s, 1H), 8.19 (s, 1H), 8.13 (dt, J=7.9, 1.4 Hz, 1H), 8.05 (s, 1H), 7.91 (t, J=1.8 Hz, 1H), 7.82-7.73 (m, 2H), 7.65-7.51 (m, 3H), 7.37 (dt, J=7.8, 1.3 Hz, 1H), 4.63 (s, 1H), 3.83 (dd, J=14.2, 14.2 Hz, 2H), 3.20 (s, 3H), 2.44-2.36 (m, 1H), 2.28-2.17 (m, 1H), 1.52-1.40 (m, 1H), 1.01 (s, 3H), 0.92 (s, 3H). Missing 2H under water peak.

### Example 268: tert-Butyl 2-(4′-fluoro-2′-(4-methyl-4H-1,2,4-triazol-3-yl)-[1,1′-biphenyl]-3-yl)-5-((((1R,2S)-2-hydroxycyclopentyl)amino)methyl)-1H-indole-1-carboxylate (GQ-6)

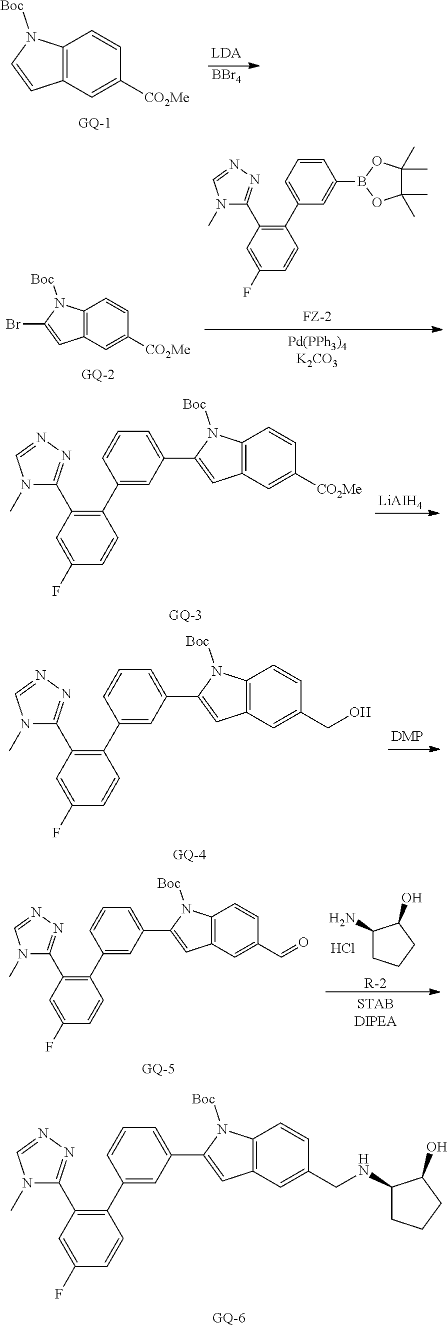

Step 1: 1-(tert-Butyl) 5-methyl 2-bromo-1H-indole-1,5-dicarboxylate (GQ-2). To a stirred solution of 1-(tert-butyl) 5-methyl 1H-indole-1,5-dicarboxylate (GQ-1) (196.6 mg, 93% Wt, 1 Eq, 664.1 μmol) in dry THF (3.0 mL) under a nitrogen atmosphere at −78° C. was added LDA solution in THF/n-heptane/ethylbenzene (393 mg, 500 μL, 2.0 molar, 1.51 Eq, 1.00 mmol). The reaction mixture was stirred at −78° C. for 30 min. CBr₄ (274.7 mg, 80.3 μL, 1.247 Eq, 828.3 μmol) in THF (1.0 mL) was added and the resulting mixture was stirred at −78° C. for 2 h. The reaction mixture was quenched with water (5 mL), extracted with EtOAc (3×5 mL). The combined organic layers were collected, dried (Na₂SO₄), filtered and concentrated in vacuo. The crude product was purified by chromatography on silica gel (12 g cartridge, 0-50% EtOAc/isohexane) to afford the sub-title compound (GQ-2) (97.3 mg, 0.19 mmol, 29%, 70% Purity) as a yellow oil. ¹H NMR (400 MHz, DMSO) δ 8.21 (dd, J=1.8, 0.6 Hz, 1H), 8.08 (dt, J=8.8, 0.7 Hz, 1H), 7.92 (dd, J=8.8, 1.8 Hz, 1H), 7.14 (d, J=0.7 Hz, 1H), 3.87 (s, 3H), 1.66 (s, 9H).

Step 2: 1-(tert-Butyl) 5-methyl 2-(4′-fluoro-2′-(4-methyl-4H-1,2,4-triazol-3-yl)-[1,1′-biphenyl]-3-yl)-1H-indole-1,5-dicarboxylate (GQ-3). A solution of intermediate (FZ-2) (64.8 mg, 90% Wt, 1 Eq, 154 μmol), the product from step 1 above (GQ-2) (97.3 mg, 70% Wt, 1.25 Eq, 192 μmol) and K₂CO₃ (89.3 mg, 4.20 Eq, 646 μmol) in 1,4-dioxane (2.0 mL) and water (0.2 mL) was degassed under a nitrogen atmosphere at rt for 10 min. Pd(PPh₃)₄ (21.3 mg, 0.120 Eq, 18.4 μmol) was added and the reaction mixture was heated at 100° C. for 3 h. The reaction mixture was cooled to rt and the volatiles were removed in vacuo. The crude product was purified by chromatography on silica gel (12 g cartridge, 0-10% MeOH/DCM) to afford the sub-title compound (GQ-3) (91.1 mg, 0.11 mmol, 69%, 61% Purity) as a yellow oil. m/z 527.2 (M+H)⁺ (ES+); m/z 427.2 (M-Boc+H)⁺ (ES+).

Step 3: tert-Butyl 2-(4′-fluoro-2′-(4-methyl-4H-1,2,4-triazol-3-yl)-[1,1′-biphenyl]-3-yl)-5-(hydroxymethyl)-1H-indole-1-carboxylate (GQ-4). To a solution of the product from step 2 above (GQ-3) (91.1 mg, 61% Wt, 1 Eq, 106 μmol) in THF (2.0 mL) was added LiAlH₄ in THF (6.83 mg, 180 μL, 1 molar, 1.71 Eq, 180 μmol) dropwise at 0° C. This mixture was stirred at 0° C. for 1 h. The mixture was quenched with NaOH (2 M, 0.5 mL) at 0° C. Na₂SO₄ was added. The mixture was warmed up to rt, stirred for 15 min, filtered and the volatiles were removed in vacuo. The crude product was purified by chromatography on silica gel (12 g cartridge, 0-10% MeOH/DCM) to afford an inseparable mixture of the sub-title compound (GQ-4) (52.5 mg, 71 μmol, 67%, 67% Purity) and des-boc variant, (2-(4′-fluoro-2′-(4-methyl-4H-1,2,4-triazol-3-yl)-[1,1′-biphenyl]-3-yl)-1H-indol-5-yl)methanol (52.5 mg, 18 μmol, 66%, 14% Purity) as a yellow oil. m/z 499.2 (M+H)⁺ (ES+); m/z 399.1 (M-Boc+H)⁺ (ES+).

Step 4: tert-Butyl 2-(4′-fluoro-2′-(4-methyl-4H-1,2,4-triazol-3-yl)-[1,1′-biphenyl]-3-yl)-5-formyl-1H-indole-1-carboxylate (GQ-5). To a stirred solution of a mixture of the product from step 3 above (GQ-4) (45.6 mg, 67% Wt, 1 Eq, 61.3 μmol) and (2-(4′-fluoro-2′-(4-methyl-4H-1,2,4-triazol-3-yl)-[1,1′-biphenyl]-3-yl)-1H-indol-5-yl)methanol (45.6 mg, 14% Wt, 0.261 Eq, 16.0 μmol) in dry DCM (3.0 mL) at rt was added Dess-Martin periodinane (59.8 mg, 2.30 Eq, 141 μmol). The reaction mixture was stirred at rt for 1 h. The reaction mixture was diluted with DCM (5 mL), washed with sat. NaHCO₃ (5 mL) and brine (5 mL). The organic layer was dried (Na₂SO₄), filtered and concentrated in vacuo. The crude product was purified by chromatography on silica gel (12 g cartridge, 0-10% MeOH/DCM) to afford the sub-title compound (GQ-5) (36.7 mg, 63 μmol, 100%, 85% Purity) as a pale-yellow solid. m/z 497.2 (M+H)⁺ (ES+). ¹H NMR (400 MHz, MeOD) δ 10.04 (s, 1H), 8.42 (s, 1H), 8.28 (d, J=8.7 Hz, 1H), 8.17 (d, J=1.6 Hz, 1H), 7.88 (dd, J=8.7, 1.7 Hz, 1H), 7.72 (dd, J=8.7, 5.5 Hz, 1H), 7.54-7.47 (m, 1H), 7.47-7.38 (m, 3H), 7.30 (dt, J=7.3, 1.7 Hz, 1H), 7.26 (t, J=1.8 Hz, 1H), 6.65 (s, 1H), 3.15 (s, 3H), 1.36 (s, 9H).

Step 5: tert-Butyl 2-(4′-fluoro-2′-(4-methyl-4H-1,2,4-triazol-3-yl)-[1,1′-biphenyl]-3-yl)-5-((((1R,2S)-2-hydroxycyclopentyl)amino)methyl)-1H-indole-1-carboxylate (GQ-6). A solution of the product from step 4 above (GQ-5) (36.7 mg, 85% Wt, 1 Eq, 62.8 μmol), (1S,2R)-2-aminocyclopentan-1-ol, HCl (R-2) (15.4 mg, 1.78 Eq, 112 μmol) and DIPEA (37 mg, 50 μL, 4.6 Eq, 0.29 mmol) in CHCl₃ (2.0 mL) was stirred at rt for 1 h. NaBH(OAc)₃ (60.5 mg, 97% Wt, 4.41 Eq, 277 μmol) was added and the reaction mixture was stirred at rt for 4 d. The reaction mixture was diluted with EtOAc (2 mL), washed with sat. NaHCO₃ (2 mL) and brine (2 mL), dried (Na₂SO₄), filtered and concentrated in vacuo. The crude product was purified by chromatography on silica gel (4 g cartridge, 0-20% MeOH/DCM) to afford the title compound (GQ-6) (10.7 mg, 18 μmol, 29%, 99% Purity) as a white solid. m/z 582.6 (M+H)⁺ (ES+). ¹H NMR (400 MHz, MeOD) δ 8.41 (s, 1H), 8.09 (d, J=8.6 Hz, 1H), 7.71 (dd, J=8.7, 5.5 Hz, 1H), 7.58 (d, J=1.7 Hz, 1H), 7.50 (td, J=8.5, 2.8 Hz, 1H), 7.45-7.32 (m, 4H), 7.27 (dt, J=7.5, 1.6 Hz, 1H), 7.23 (t, J=1.7 Hz, 1H), 6.46 (d, J=0.8 Hz, 1H), 4.23-4.16 (m, 1H), 4.01 (d, J=12.7 Hz, 1H), 3.92 (d, J=12.7 Hz, 1H), 3.14 (s, 3H), 3.07-3.00 (m, 1H), 1.99-1.90 (m, 1H), 1.88-1.79 (m, 2H), 1.79-1.69 (m, 1H), 1.63-1.52 (m, 2H), 1.35 (s, 9H). 2× Exchangeable protons not observed.

### Example 269: (1S,3S)-3-(((2-(4′-fluoro-2′-(4-methyl-4H-1,2,4-triazol-3-yl)-[1,1′-biphenyl]-3-yl)-7-(trifluoromethyl)benzo[d]oxazol-5-yl)methyl)amino)cyclobutan-1-ol, Formic Acid (GR-2)

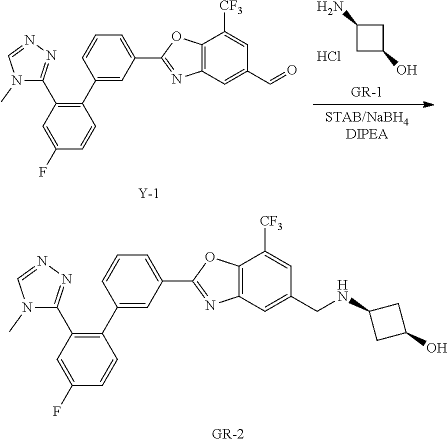

NaBH(OAc)₃ (37 mg, 3.0 Eq, 0.17 mmol) was added to a stirred mixture of intermediate (Y-1) (30 mg, 90% Wt, 1 Eq, 58 μmol), (1S,3S)-3-aminocyclobutan-1-ol, HCl (GR-1) (14 mg, 2.0 Eq, 0.12 mmol) and DIPEA (22 mg, 31 μL, 3.0 Eq, 0.17 mmol) in CHCl₃ (3 mL). The reaction mixture was stirred at rt overnight. NaBH₄ (22 mg, 10.0 Eq, 0.58 mmol) was added to the reaction mixture and stirring continued at rt over the weekend. More NaBH₄ (22 mg, 10.0 Eq, 0.58 mmol) was added to the reaction mixture and stirring continued at 40° C. overnight. The reaction mixture was diluted with DCM (4 mL) and washed with sat. aq. NaHCO₃ (10 mL). The organic phase was separated and evaporated to afford the crude product. The crude product was purified by preparative HPLC (Waters, acidic (0.1% formic acid), acidic, Waters X-Select CSH C18 ODB prep column, 130A, 5 μm, 30 mm×100 mm column, 5-100% MeCN in water) to afford the title compound (GR-2) (4 mg, 7 μmol, 10%, 98% Purity) as a colourless foam. m/z 538.4 (M+H)⁺ (ES+). ¹H NMR (400 MHz, MeOD) δ 8.49 (s, 1H), 8.41 (s, 1H), 8.28-8.21 (m, 1H), 8.15-8.05 (m, 2H), 7.87 (s, 1H), 7.81 (dd, J=8.6, 5.5 Hz, 1H), 7.67-7.54 (m, 2H), 7.54-7.47 (m, 2H), 4.22 (s, 2H), 4.08 (p, J=7.4 Hz, 1H), 3.28 (s, 3H), 3.23 (dd, J=8.9, 7.0 Hz, 1H), 2.73 (dtd, J=9.8, 6.9, 3.0 Hz, 2H), 2.06-1.94 (m, 2H). Note formate salt singlet @8.49 ppm. Exchangeable protons not observed.

### Example 270: (1R,3R)-3-(((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)amino)cyclobutan-1-ol, Formic Acid (GS-2)

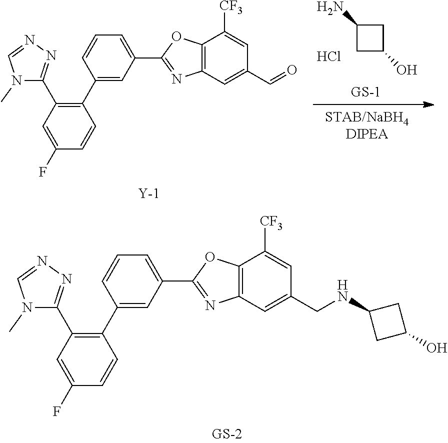

NaBH(OAc)₃ (37 mg, 3.0 Eq, 0.17 mmol) was added to a stirred mixture of intermediate (Y-1) (30 mg, 90% Wt, 1 Eq, 58 μmol), (1R,3R)-3-aminocyclobutan-1-ol, HCl (GS-1) (14 mg, 2.0 Eq, 0.12 mmol) and DIPEA (22 mg, 31 μL, 3.0 Eq, 0.17 mmol) in CHCl₃ (3 mL). The reaction mixture was stirred at rt overnight. More DIPEA (22 mg, 31 μL, 3.0 Eq, 0.17 mmol) was added and the reaction mixture sonicated. The reaction was left to stir at rt over the weekend. NaBH₄ (22 mg, 10.0 Eq, 0.58 mmol) was added to the reaction mixture and stirring continued at 40° C. overnight. The reaction mixture was diluted with DCM (4 mL) and washed with sat. aq. NaHCO₃ (10 mL). The organic phase was separated and evaporated to afford the crude product. The crude product was purified by preparative HPLC (Waters, acidic (0.1% formic acid), acidic, Waters X-Select CSH C18 ODB prep column, 130A, 5 μm, 30 mm×100 mm column, 5-100% MeCN in water) to afford the title compound (GS-2) (4 mg, 7 μmol, 10%, 97% Purity) as a colourless foam. m/z 538.8 (M+H)⁺ (ES+). ¹H NMR (400 MHz, DMSO) δ 8.46 (s, 1H), 8.38 (s, 1H), 8.22 (d, J=7.8 Hz, 1H), 8.10 (s, 1H), 8.05 (s, 1H), 7.84 (s, 1H), 7.79-7.74 (m, 1H), 7.63-7.51 (m, 2H), 7.50-7.44 (m, 2H), 4.53-4.44 (m, 1H), 4.20 (s, 2H), 3.88-3.80 (m, 1H), 3.25 (s, 3H), 2.50-2.39 (m, 2H), 2.35-2.24 (m, 2H). Note formate salt singlet @8.46 ppm. Exchangeable protons not observed.

### Example 271: (1S,2R)-2-(((2-(4′-Fluoro-2′-(4-methyl-4H-1,2,4-triazol-3-yl)-[1,1′-biphenyl]-3-yl)-1H-indol-5-yl)methyl)amino)cyclopentan-1-ol (GT-1)

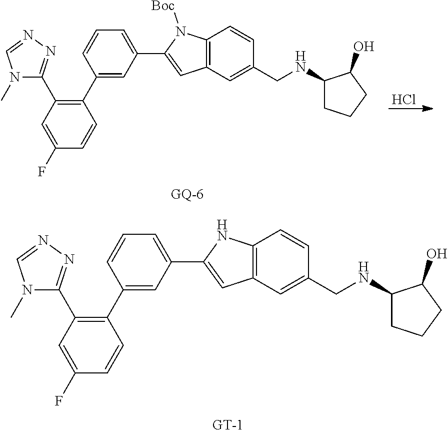 [SMILES: Cn1cnnc1-c1cc(F)ccc1-c1cccc(-c2cc3cc(CN[C@@H]4CCC[C@@H]4O)ccc3n2C(=O)OC(C)(C)C)c1 ; Cl ; Cn1cnnc1-c1cc(F)ccc1-c1cccc(-c2cc3cc(CN[C@@H]4CCC[C@@H]4O)ccc3[nH]2)c1]

To a stirred solution of compound (GQ-6) (6.8 mg, 99% Wt, 1 Eq, 12 μmol) in dry DCM at rt was added HCl in 1,4-dioxane (45 mg, 30 μL, 4.0 molar, 10 Eq, 0.12 mmol). The reaction mixture was stirred at rt for 4 h. The reaction was quenched with 0.7 M NH₃ in MeOH. The volatiles were removed in vacuo. The crude product was purified by chromatography on silica gel (4 g cartridge, 0-50% MeOH/DCM) to afford the title compound (GT-1) (3.3 mg, 6.0 μmol, 52%, 88% Purity) as a white solid. m/z 482.3 (M+H)⁺ (ES+). ¹H NMR (400 MHz, MeOD) δ 8.36 (s, 1H), 7.76 (dd, J=8.5, 5.8 Hz, 2H), 7.68-7.64 (m, 1H), 7.61-7.57 (m, 1H), 7.56-7.50 (m, 1H), 7.48-7.41 (m, 3H), 7.20 (ddd, J=13.1, 8.0, 1.7 Hz, 2H), 6.72 (s, 1H), 4.39-4.32 (m, 1H), 4.27 (d, J=12.9 Hz, 1H), 4.19 (d, J=12.9 Hz, 1H), 3.40-3.33 (m, 1H), 3.12 (s, 3H), 2.11-2.01 (m, 1H), 2.01-1.75 (m, 4H), 1.70-1.57 (m, 1H). 3× exchangeable protons not observed.

### Example 272: 1-Cyclobutyl-N-((2-(4′-fluoro-2′-(4-methyl-4H-1,2,4-triazol-3-yl)-[1,1′-biphenyl]-3-yl)-7-methylbenzo[d]oxazol-5-yl)methyl)methanamine (GU-1)

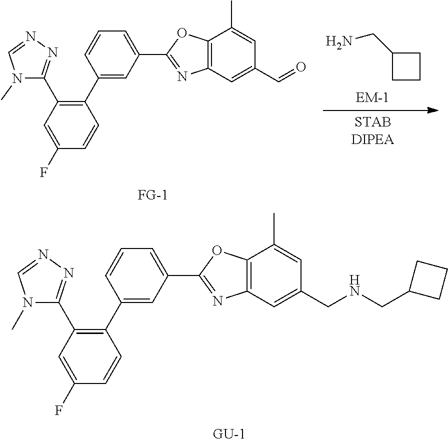 [SMILES: Cc1cc(C=O)cc2nc(-c3cccc(-c4ccc(F)cc4-c4nncn4C)c3)oc12 ; NCC1CCC1 ; "FG-1" ; Cc1cc(CNCC2CCC2)cc2nc(-c3cccc(-c4ccc(F)cc4-c4nncn4C)c3)oc12]

A solution of intermediate (FG-1) (26.0 mg, 85% Wt, 1 Eq, 53.6 μmol), (cyclobutylmethyl)amine (EM-1) (12.1 mg, 2.65 Eq, 142 μmol) and DIPEA (41 mg, 55 μL, 5.9 Eq, 0.32 mmol) in CHCl₃ (3.0 mL) was stirred at rt for 1 h. NaBH(OAc)₃ (43.5 mg, 97% Wt, 3.72 Eq, 199 μmol) was added and the reaction mixture was stirred at rt for 3 d. Additional (cyclobutylmethyl)amine (EM-1) (5.0 mg, 1.1 Eq, 59 μmol) and STAB (38.7 mg, 97% Wt, 3.31 Eq, 177 μmol) was added and the resulting mixture was stirred at rt for 3 h. The reaction mixture was diluted with EtOAc (2 mL), washed with sat. NaHCO₃ (2 mL) and brine (2 mL), dried (Na₂SO₄), filtered and concentrated in vacuo. The crude product was purified by chromatography on silica gel (4 g cartridge, 0-20% MeOH/DCM) to afford the title compound (GU-1) (11.6 mg, 24 μmol, 45%, 99% Purity) as a white solid. m/z 482.3 (M+H)⁺ (ES+). ¹H NMR (400 MHz, MeOD) δ 8.38 (s, 1H), 8.22-8.18 (m, 1H), 8.06-8.03 (m, 1H), 7.79 (dd, J=8.7, 5.4 Hz, 1H), 7.59-7.52 (m, 3H), 7.46 (dd, J=8.8, 2.7 Hz, 1H), 7.44-7.40 (m, 1H), 7.26-7.23 (m, 1H), 3.87 (s, 2H), 3.18 (s, 3H), 2.67 (d, J=7.4 Hz, 2H), 2.60 (s, 3H), 2.59-2.51 (m, 1H), 2.15-2.06 (m, 2H), 1.98-1.79 (m, 2H), 1.76-1.66 (m, 2H). 1× exchangeable NH proton not observed.

### Example 273: N-((2-(4′-Fluoro-2′-(4-methyl-4H-1,2,4-triazol-3-yl)-[1,1′-biphenyl]-3-yl)-7-methylbenzo[d]oxazol-5-yl)methyl)-2-methoxyethan-1-amine (GV-1)

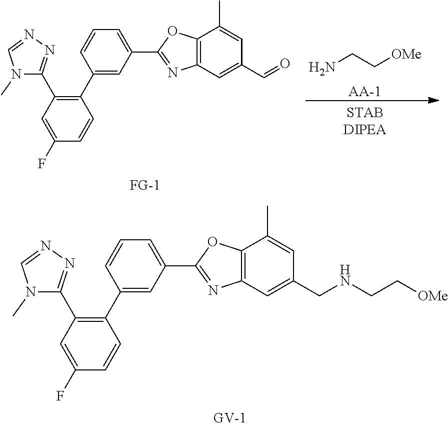

A solution of intermediate (FG-1) (25.4 mg, 85% Wt, 1 Eq, 52.3 μmol), 1-amino-2-methoxyethane (AA-1) (10 mg, 12 μL, 2.6 Eq, 0.14 mmol) and DIPEA (41 mg, 55 μL, 6.0 Eq, 0.32 mmol) in CHCl₃ (3.0 mL) was stirred at rt for 1 h. NaBH(OAc)₃ (44.0 mg, 97% Wt, 3.85 Eq, 201 μmol) was added and the reaction mixture was stirred at rt for 3 d. Additional 1-amino-2-methoxyethane (AA-1) (5 mg, 6 μL, 1 Eq, 0.07 mmol) and NaBH(OAc)₃ (38.2 mg, 97% Wt, 3.34 Eq, 175 μmol) was added and the resulting mixture was stirred at rt for 3 h. The reaction mixture was diluted with EtOAc (2 mL), washed with sat. NaHCO₃ (2 mL) and brine (2 mL), dried (Na₂SO₄), filtered and concentrated in vacuo. The crude product was purified by chromatography on silica gel (4 g cartridge, 0-20% MeOH/DCM) to afford the title compound (GV-1) (11.7 mg, 25 μmol, 47%, 99% Purity) as a white solid. m/z 472.2 (M+H)⁺ (ES+). ¹H NMR (400 MHz, MeOD) δ 8.38 (s, 1H), 8.22-8.18 (m, 1H), 8.07-8.03 (m, 1H), 7.79 (dd, J=8.7, 5.4 Hz, 1H), 7.59-7.51 (m, 3H), 7.46 (dd, J=8.9, 2.7 Hz, 1H), 7.44-7.39 (m, 1H), 7.25 (s, 1H), 3.89 (s, 2H), 3.53 (t, J=5.4 Hz, 2H), 3.35 (s, 3H), 3.18 (s, 3H), 2.79 (t, J=5.3 Hz, 2H), 2.60 (s, 3H). 1× Exchangeable NH proton not observed.

### Example 274: (S)-1-Cyclobutyl-N-((2-(4′-fluoro-2′-(4-methyl-4H-1,2,4-triazol-3-yl)-[1,1′-biphenyl]-3-yl)-7-(trifluoromethyl)benzo[d]oxazol-5-yl)methyl)ethan-1-amine (GW-2)

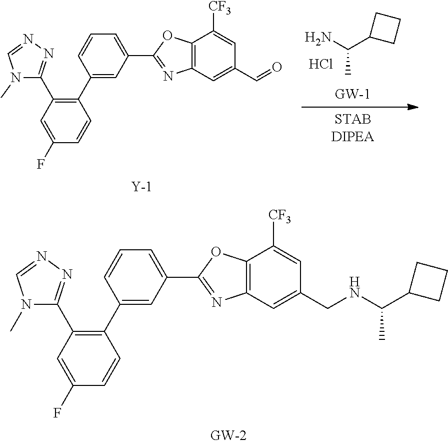

A solution of intermediate (Y-1) (30.1 mg, 98% Wt, 1 Eq, 63.2 μmol), (1S)-1-cyclobutylethan-1-amine, HCl (GW-1) (16.3 mg, 95% Wt, 1.81 Eq, 114 μmol) and DIPEA (41 mg, 55 μL, 5.0 Eq, 0.32 mmol) in CHCl₃ (2.0 mL) was stirred at rt for 1 h. NaBH(OAc)₃ (42.3 mg, 97% Wt, 3.06 Eq, 194 μmol) was added and the reaction mixture was stirred at rt overnight. NaBH(OAc)₃ (40.7 mg, 97% Wt, 2.95 Eq, 186 μmol) was added and the resulting mixture was stirred at rt for 2 h. NaBH(OAc)₃ (40.1 mg, 97% Wt, 2.90 Eq, 184 μmol) was added and the reaction mixture was stirred at rt for 1 h. The reaction mixture was diluted with EtOAc (5 mL), washed with sat. NaHCO₃ (5 mL) and brine (5 mL), dried (Na₂SO₄), filtered and concentrated in vacuo. The crude product was purified by chromatography on silica gel (4 g cartridge, 0-10% MeOH/DCM) to afford the desired product with ˜90% purity. The crude product was purified by preparative HPLC (Waters, basic (0.3% ammonia), basic, Waters XBridge BEH C18 ODB, 5 μm, 30×100 mm column, 50-100% MeCN in water) to afford the title compound (GW-2) (6.0 mg, 11 μmol, 17%, 99% Purity) as a white solid. m/z 550.2 (M+H)⁺ (ES+). ¹H NMR (400 MHz, MeOD) δ 8.38 (s, 1H), 8.21 (dt, J=7.9, 1.3 Hz, 1H), 8.06 (t, J=1.7 Hz, 1H), 7.99 (s, 1H), 7.78 (dd, J=8.6, 5.5 Hz, 1H), 7.75 (s, 1H), 7.61-7.51 (m, 2H), 7.49-7.41 (m, 2H), 4.00 (d, J=13.6 Hz, 1H), 3.89 (d, J=13.6 Hz, 1H), 3.24 (s, 3H), 2.61-2.52 (m, 1H), 2.36-2.24 (m, 1H), 2.15-2.06 (m, 1H), 2.01-1.94 (m, 1H), 1.93-1.83 (m, 1H), 1.80-1.64 (m, 3H), 1.01 (d, J=6.3 Hz, 3H). 1× Exchangeable NH proton not observed.

### Example 275: (1S,3S)-3-(((2-(4′-Fluoro-2′-(4-methyl-4H-1,2,4-triazol-3-yl)-[1,1′-biphenyl]-3-yl)-7-methoxybenzo[d]oxazol-5-yl)methyl)amino)cyclobutan-1-ol (GX-1)

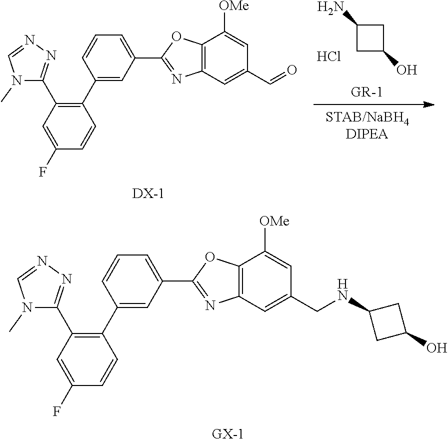

A mixture of intermediate (DX-1) (30 mg, 90% Wt, 1 Eq, 63 μmol), (1S,3S)-3-aminocyclobutan-1-ol, HCl (GR-1) (16 mg, 2.0 Eq, 0.13 mmol) and DIPEA (24 mg, 34 μL, 3.0 Eq, 0.19 mmol) in CHCl₃ (3 mL) was stirred at 40° C. for 90 min. NaBH(OAc)₃ (40 mg, 3.0 Eq, 0.19 mmol) was added and the reaction mixture was stirred at 40° C. overnight. NaBH₄ (24 mg, 10.0 Eq, 0.63 mmol) was added to the reaction mixture and stirring continued at 40° C. for an additional 2 hours. The reaction mixture was quenched with water (0.5 mL) and allowed to stand at rt over the weekend. The reaction mixture was diluted with DCM (4 mL) and washed with sat. aq. NaHCO₃ (10 mL). The organic phase was separated and evaporated to afford the crude product. The crude product was purified by preparative HPLC (Waters, Basic (0.3% ammonia), Basic, Waters X-Bridge BEH C18 ODB Prep-C18, 5 μm, 30×100 mm column, 20-100% MeCN in water) to afford the title compound (GX-1) (7 mg, 0.01 mmol, 20%, 99% Purity) as a beige foam. m/z 500.6 (M+H)⁺ (ES+). ¹H NMR (400 MHz, DMSO) δ 8.41 (s, 1H), 8.10 (d, J=7.8 Hz, 1H), 7.96 (s, 1H), 7.80-7.75 (m, 1H), 7.64-7.56 (m, 1H), 7.56-7.47 (m, 2H), 7.31-7.23 (m, 2H), 7.05 (s, 1H), 4.86 (d, J=6.0 Hz, 1H), 4.00 (s, 3H), 3.70 (s, 2H), 3.13 (s, 3H), 2.65-2.61 (m, 1H), 2.42-2.38 (m, 2H), 2.25-2.21 (m, 1H), 1.58-1.52 (m, 2H). Missing exchangeable proton not observed.

### Example 276: (1R,3R)-3-(((2-(4′-Fluoro-2′-(4-methyl-4H-1,2,4-triazol-3-yl)-[1,1′-biphenyl]-3-yl)-7-methoxybenzo[d]oxazol-5-yl)methyl)amino)cyclobutan-1-ol (GY-1)

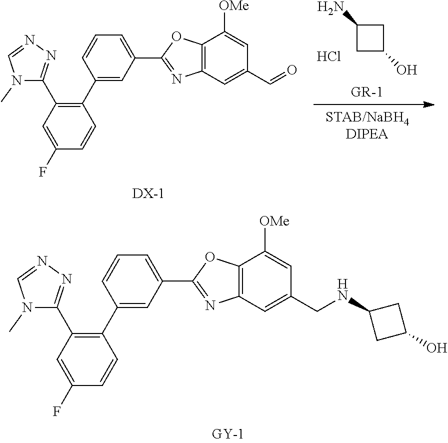

A mixture of intermediate (DX-1) (30 mg, 90% Wt, 1 Eq, 63 μmol), (1R,3R)-3-aminocyclobutan-1-ol, HCl (GS-1) (16 mg, 2.0 Eq, 0.13 mmol) and DIPEA (24 mg, 34 μL, 3.0 Eq, 0.19 mmol) in CHCl₃ (3 mL) was stirred at 40° C. for 90 min. NaBH(OAc)₃ (40 mg, 3.0 Eq, 0.19 mmol) was added and the reaction mixture was stirred at 40° C. overnight. NaBH₄ (24 mg, 10.0 Eq, 0.63 mmol) was added to the reaction mixture and stirring continued at 40° C. for an additional 2 h. The reaction mixture was quenched with water (0.5 mL) and allowed to stand at rt over the weekend. The reaction mixture was diluted with DCM (4 mL) and washed with sat. aq. NaHCO₃ (10 mL). The organic phase was separated and evaporated to afford the crude product. The crude product was purified by preparative HPLC (Waters, Basic (0.3% ammonia), Basic, Waters X-Bridge BEH C18 ODB Prep-C18, 5 μm, 30×100 mm column, 20-100% MeCN in water) to afford the title compound (GY-1) (4 mg, 8 μmol, 10%, 99% Purity) as a colourless glass. m/z 501.4 (M+H)⁺ (ES+). ¹H NMR (400 MHz, MeOD) δ 8.40 (s, 1H), 8.20 (dt, J=8.1, 1.3 Hz, 1H), 8.10 (t, J=1.7 Hz, 1H), 7.80 (dd, J=8.6, 5.5 Hz, 1H), 7.62-7.52 (m, 2H), 7.48 (dd, J=8.8, 2.7 Hz, 1H), 7.40 (dt, J=7.9, 1.4 Hz, 1H), 7.31 (d, J=1.3 Hz, 1H), 7.07 (d, J=1.3 Hz, 1H), 4.45 (ddd, J=11.3, 6.7, 4.5 Hz, 1H), 4.10 (s, 3H), 3.80 (s, 2H), 3.56-3.45 (m, 1H), 3.22 (s, 3H), 2.22-2.12 (m, 4H). Two exchangeable protons not observed.

### Example 277: 1-(2-(4′-Fluoro-2′-(4-methyl-4H-1,2,4-triazol-3-yl)-[1,1′-biphenyl]-3-yl)-7-methoxybenzo[d]oxazol-5-yl)-N-(oxetan-3-ylmethyl)methanamine (GZ-1)

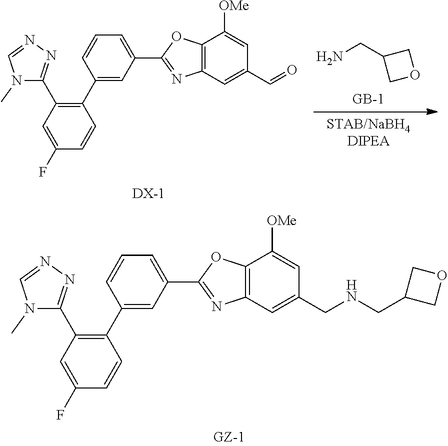

A mixture of intermediate (DX-1) (30 mg, 1 Eq, 70 μmol) and oxetan-3-ylmethanamine (GB-1) (18 mg, 3.0 Eq, 0.21 mmol) in CHCl₃ (3 mL) were stirred at 40° C. for 90 min. NaBH(OAc)₃ (45 mg, 3.0 Eq, 0.21 mmol) was added and the reaction mixture was stirred at 40° C. overnight. NaBH₄ (26 mg, 10.0 Eq, 0.70 mmol) was added to the reaction mixture and stirring continued at 40° C. for an additional 2 hours. The reaction mixture was quenched with water (0.5 mL) and allowed to stand at rt over the weekend. The reaction mixture was diluted with DCM (4 mL) and washed with sat. aq. NaHCO₃ (10 mL). The organic phase was separated and evaporated to afford the crude product. The crude product was purified by preparative HPLC (Waters, Basic (0.3% ammonia), Basic, Waters X-Bridge BEH C18 ODB Prep-C18, 5 μm, 30×100 mm column, 15-100% MeCN in water) to afford the title compound (GZ-1) (11 mg, 22 μmol, 31%, 100% Purity) as a white foam. m/z 500.6 (M+H)⁺ (ES+). ¹H NMR (400 MHz, DMSO) δ 8.41 (s, 1H), 8.10 (dt, J=8.0, 1.3 Hz, 1H), 7.96 (t, J=1.8 Hz, 1H), 7.78 (dd, J=8.6, 5.7 Hz, 1H), 7.60 (td, J=8.6, 2.8 Hz, 1H), 7.56-7.48 (m, 2H), 7.32 (d, J=1.3 Hz, 1H), 7.27 (dt, J=8.0, 1.3 Hz, 1H), 7.07 (d, J=1.3 Hz, 1H), 4.61 (dd, J=7.7, 5.9 Hz, 2H), 4.25 (t, J=5.9 Hz, 2H), 4.01 (s, 3H), 3.78 (s, 2H), 3.13 (s, 3H), 3.09-2.98 (m, 1H), 2.76 (d, J=7.4 Hz, 2H). Missing exchangeable proton not observed.

### Example 278: (1S,2R)-2-(((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl-d2)amino)cyclopentan-1-ol (HA-3)

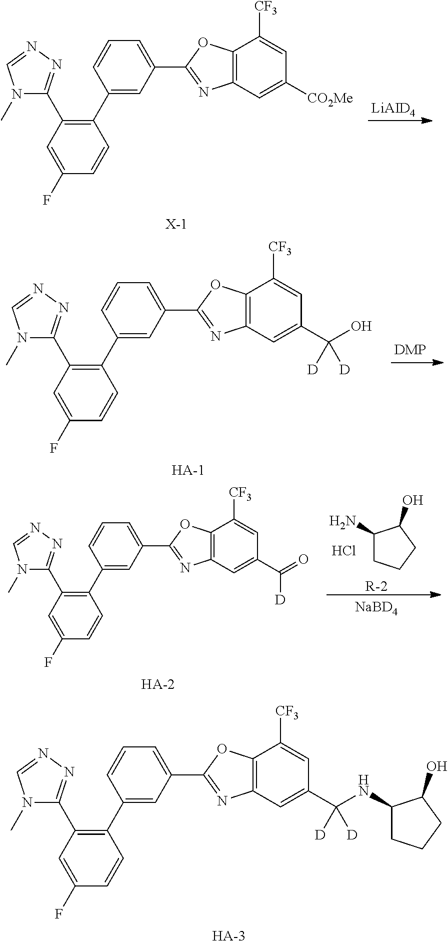

Step 1: (2-(4′-Fluoro-2′-(4-methyl-4H-1,2,4-triazol-3-yl)-[1,1′-biphenyl]-3-yl)-7-(trifluoromethyl)benzo[d]oxazol-5-yl)methan-d2-ol (HA-1). To a solution of intermediate (X-1) (389 mg, 85% Wt, 1 Eq, 666 μmol) in anhydrous THF (4 mL) was added LiAlD₄ (30.8 mg, 1.1 Eq, 733 μmol) in THF (1 mL) dropwise at 0° C. under N₂. The mixture was stirred at this temperature for 4 h. The reaction mixture was diluted with EtOAc (20 mL) and quenched with water (5 mL), filtered, dried (phase separator) and concentrated in vacuo. The crude product was purified by chromatography on silica gel (12 g cartridge, 0-10% MeOH/DCM) to afford the sub-title compound (HA-1) (128.4 mg, 0.27 mmol, 40%, 98% Purity) as a brown oil. m/z 471.1 (M+H)⁺ (ES+)

Step 2: 2-(4′-Fluoro-2′-(4-methyl-4H-1,2,4-triazol-3-yl)-[1,1′-biphenyl]-3-yl)-7-(trifluoromethyl)benzo[d]oxazole-5-carbaldehyde-d (HA-2). The product from step 1 above (HA-1) (128 mg, 98% Wt, 1 Eq, 267 μmol) and Dess-Martin periodinane (170 mg, 1.5 Eq, 400 μmol) were stirred in DCM (5 mL) at rt for 1 h. The reaction mixture was diluted with DCM (50 mL) and washed with sat. aq. NaHCO₃ (20 mL) and brine (20 mL). Organics were separated, dried (phase separator), and evaporated in vacuo. The crude product was purified by chromatography on silica gel (12 g cartridge, 0-10% MeOH/DCM) to afford the sub-title compound (HA-2) (124.4 mg, 0.25 mmol, 95%, 95% Purity) as a pale tan solid. m/z 468.1 (M+H)⁺ (ES+). ¹H NMR (400 MHz, DMSO) δ 8.66 (s, 1H), 8.42 (s, 1H), 8.34 (s, 1H), 8.18 (dt, J=7.7, 1.4 Hz, 1H), 7.95 (t, J=1.9 Hz, 1H), 7.82-7.75 (m, 1H), 7.65-7.53 (m, 3H), 7.44-7.39 (m, 1H), 3.22 (s, 3H).

Step 3: (1S,2R)-2-(((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl-d2)amino)cyclopentan-1-ol (HA-3). A solution of the product from step 2 above (HA-2) (30 mg, 95% Wt, 1 Eq, 61 μmol), DIPEA (24 mg, 32 μL, 3.0 Eq, 0.18 mmol) and (1S,2R)-2-aminocyclopentan-1-ol, HCl (R-2) (17 mg, 2 Eq, 0.12 mmol) in EtOH (3 mL) was stirred for 4 h at rt. NaBD₄ (5.1 mg, 4.8 μL, 2 Eq, 0.12 mmol) was added to the reaction mixture and stirred for 16 h at rt. The reaction mixture was quenched with MeOH (10 mL) and concentrated in vacuo. The residue was dissolved in DCM (10 mL) and washed with sat. aq. sol. of NaHCO₃ (2×10 mL). The organic layer was extracted, dried (phase separator) and concentrated in vacuo. The crude product was purified by chromatography on silica gel (4 g cartridge, 0-10% MeOH/DCM) to afford the title compound (HA-3) (5.98 mg, 11 μmol, 17%, 98% Purity) as a pale-yellow solid. m/z 554.2 (M+H)⁺ (ES+). ¹H NMR (400 MHz, DMSO) δ 8.42 (s, 1H), 8.15-8.11 (m, 1H), 8.10 (s, 1H), 7.90 (t, J=1.8 Hz, 1H), 7.81 (s, 1H), 7.80-7.74 (m, 1H), 7.64-7.53 (m, 3H), 7.40-7.36 (m, 1H), 4.00-3.95 (m, 1H), 3.20 (s, 3H), 2.84-2.77 (m, 1H), 1.76-1.53 (m, 4H), 1.48-1.37 (m, 2H). Two exchangeable protons not observed

### Example 279: 1-((((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl-d2)amino)methyl)cyclobutan-1-ol (HB-1)

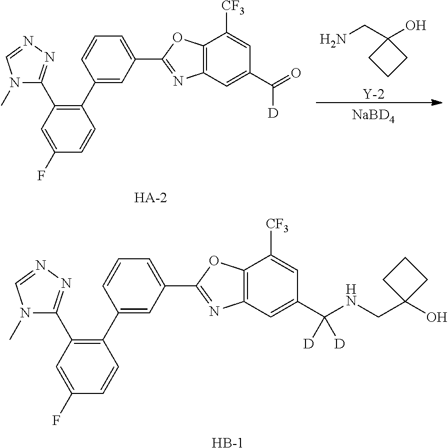

A solution of intermediate (HA-2) (30 mg, 95% Wt, 1 Eq, 61 μmol), DIPEA (24 mg, 32 μL, 3.0 Eq, 0.18 mmol) and 1-(aminomethyl)cyclobutan-1-ol (Y-2) (12 mg, 2 Eq, 0.12 mmol) in EtOH (3 mL) was stirred for 4 h at rt. NaBD₄ (5.1 mg, 4.8 μL, 2 Eq, 0.12 mmol) was added to the reaction mixture and stirred for 16 h at rt. The reaction mixture was quenched with MeOH (5 mL) and concentrated in vacuo. The crude was dissolved in DCM and washed with NaHCO₃ (3×10 mL), the organic layer was extracted, dried (phase separator) and concentrated in vacuo. The crude product was purified by chromatography on silica gel (4 g cartridge, 0-10% MeOH/DCM) to afford the title compound (HB-1) (14.99 mg, 26 μmol, 43%, 96% Purity) as an off-white solid. m/z 554.2 (M+H)⁺ (ES+). ¹H NMR (400 MHz, DMSO) δ 8.42 (s, 1H), 8.15-8.11 (m, 1H), 8.09 (d, J=1.3 Hz, 1H), 7.93-7.90 (m, 1H), 7.82-7.75 (m, 2H), 7.65-7.53 (m, 3H), 7.40-7.35 (m, 1H), 3.20 (s, 3H), 2.56 (s, 2H), 2.05-1.96 (m, 2H), 1.95-1.85 (m, 2H), 1.67-1.55 (m, 1H), 1.45-1.32 (m, 1H). Two exchangeable protons not observed

### Example 280: 2-(Dimethylamino)-N-((2-(4′-fluoro-2′-(4-methyl-4H-1,2,4-triazol-3-yl)-[1,1′-biphenyl]-3-yl)-7-(trifluoromethyl)benzo[d]oxazol-5-yl)methyl)acetamide (HC-2)

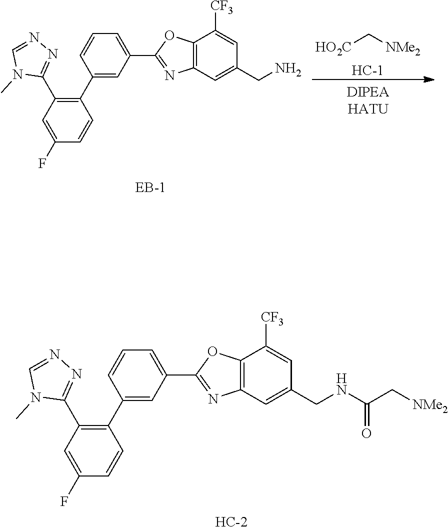

To a stirred solution intermediate (EB-1) (50 mg, 1 Eq, 0.11 mmol) in dry DMF (2 mL) under a nitrogen atmosphere at 25° C. was added DIPEA (41 mg, 56 μL, 3 Eq, 0.32 mmol), HATU (61 mg, 1.5 Eq, 0.16 mmol), and dimethylglycine (HC-1) (22 mg, 2 Eq, 0.21 mmol). The reaction mixture was stirred for 12 h. The reaction mixture was diluted with distilled water (5 mL) and transferred into a separating funnel. The layer was extracted with EtOAc (3×5 mL). The combined extracted layers were collected, dried (Na₂SO₄), filtered and concentrated in vacuo. The crude product was purified by chromatography on silica gel (12 g cartridge, 0-10% (0.7 M NH₃/MeOH)/DCM) to afford the title compound (HC-2) (20 mg, 35 μmol, 33%, 98% Purity) as a clear white solid. m/z 553.2 (M+H)⁺ (ES+). ¹H NMR (400 MHz, MeOD) δ=8.41 (s, 1H), 8.25-8.20 (m, 1H), 8.07 (t, J=1.9 Hz, 1H), 7.96 (s, 1H), 7.80 (dd, J=8.7, 5.4 Hz, 1H), 7.71 (s, 1H), 7.64-7.53 (m, 2H), 7.52-7.42 (m, 2H), 4.61 (s, 2H), 3.27 (s, 3H), 3.08 (s, 2H), 2.34 (s, 6H). One exchangeable proton is not observed.

### Example 281: N-(2-(((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)amino)ethyl)acetamide (HD-2)

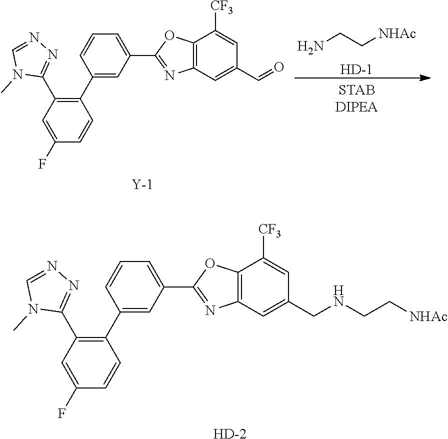

A solution of intermediate (Y-1) (50 mg, 1 Eq, 0.11 mmol), N-(2-aminoethyl)acetamide (HD-1) (22 mg, 2.1 μL, 2 Eq, 0.21 mmol) and DIPEA (42 mg, 56 μL, 3 Eq, 0.32 mmol) in CHCl₃ (10 mL) was stirred for 1 h at rt. NaBH(OAc)₃ (91 mg, 4 Eq, 0.43 mmol) was added and the reaction mixture was stirred at rt overnight. The reaction mixture was diluted with NaHCO₃ (10 mL) and transferred into a separating funnel. The layer was extracted with DCM (3×5 mL). The combined extracted layers were collected, dried (Na₂SO₄), filtered and concentrated in vacuo. The crude product was purified by chromatography on silica gel (12 g cartridge, 0-10% MeOH/DCM) to afford the title compound (HD-2) (30 mg, 53 μmol, 50%, 98% Purity) as a clear white solid. m/z 553.2 (M+H)⁺ (ES+). ¹H NMR (400 MHz, MeOD) δ=8.41 (s, 1H), 8.24 (dt, J=7.8, 1.4 Hz, 1H), 8.09 (t, J=1.7 Hz, 1H), 8.03 (s, 1H), 7.81 (dd, J=8.6, 5.5 Hz, 1H), 7.77 (s, 1H), 7.64-7.54 (m, 2H), 7.53-7.43 (m, 2H), 3.99 (s, 2H), 3.38 (d, J=6.4 Hz, 2H), 3.27 (s, 3H), 2.77 (t, J=6.4 Hz, 2H), 1.97 (s, 3H). Two exchangeable protons not observed.

### Example 282: 1-(Azetidin-1-yl)-2-(((2-(4′-fluoro-2′-(4-methyl-4H-1,2,4-triazol-3-yl)-[1,1′-biphenyl]-3-yl)-7-(trifluoromethyl)benzo[d]oxazol-5-yl)methyl)amino)ethan-1-one (HE-4)

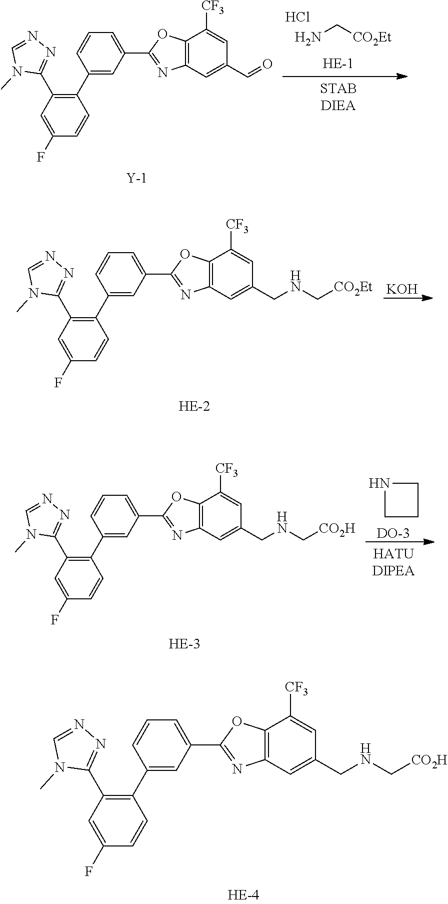

Step 1: Ethyl ((2-(4′-fluoro-2′-(4-methyl-4H-1,2,4-triazol-3-yl)-[1,1′-biphenyl]-3-yl)-7-(trifluoromethyl)benzo[d]oxazol-5-yl)methyl)glycinate (HE-2). A solution of intermediate (Y-1) (100 mg, 1 Eq, 214 μmol), ethyl glycinate, HCl (HE-1) (59.9 mg, 2 Eq, 429 μmol) and DIPEA (83.1 mg, 112 μL, 3 Eq, 643 μmol) in CHCl₃ (10 mL) was stirred for 1 h at rt. NaBH(OAc)₃ (182 mg, 4 Eq, 858 μmol) was added and the reaction mixture was stirred at rt overnight. The reaction mixture was diluted with NaHCO₃ (10 mL) and transferred into a separating funnel. The layer was extracted with DCM (3×5 mL). The combined extracted layers were collected, dried (Na2SO₄), filtered and concentrated in vacuo. The crude product was purified by chromatography on silica gel (24 g cartridge, 0-10% MeOH/DCM) to afford the sub-title compound (HE-2) (30 mg, 51 μmol, 24%, 94% Purity) as a clear white solid. m/z 554.2 (M+H)⁺ (ES+).

Step 2: ((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)glycine (HE-3). The product from step 1 above (HE-2) (30 mg, 1 Eq, 54 μmol) was combined with KOH (7.6 mg, 2.5 Eq, 0.14 mmol) in THF (2 mL) and water (1 mL). The resultant biphasic mixture was stirred at 50° C. for 12 h. The mixture was diluted with water (40 mL) and concentrated in vacuo to ˜30 ml volume, then acidified using 1 M HCl (aq) (12 mL). The resultant white precipitate was collected by filtration, washing with water (2×25 mL) and dried in vacuo. The resultant highly hygroscopic white solid was triturated with MeCN (50 mL) and concentrated in vacuo to afford the sub-title compound (HE-3) (20 mg, 29 μmol, 53%, 76% Purity) as a white powder. m/z 526.1 (M+H)⁺ (ES+).

Step 3: 1-(Azetidin-1-yl)-2-(((2-(4′-fluoro-2′-(4-methyl-4H-1,2,4-triazol-3-yl)-[1,1′-biphenyl]-3-yl)-7-(trifluoromethyl)benzo[d]oxazol-5-yl)methyl)amino)ethan-1-one (HE-4). To a stirred solution the product from step 2 above (HE-3) (20 mg, 1 Eq, 38 μmol) in dry DMF (2 mL) under a nitrogen atmosphere at 25° C. was added DIPEA (15 mg, 20 μL, 3 Eq, 0.11 mmol), HATU (22 mg, 1.5 Eq, 0.057 mmol), and azetidine (DO-3) (8.7 mg, 4 Eq, 0.15 mmol). The reaction mixture was stirred for 12 hours. The reaction mixture was diluted with cold water (10 mL) and transferred into a separating funnel. The layer was extracted with DCM (3×10 mL). The combined extracted layers were collected, dried (Na₂SO₄), filtered and concentrated in vacuo. The crude product was purified by chromatography on silica gel (12 g cartridge, 0-10% MeOH/DCM) to afford the title compound (HE-4) (7 mg, 0.01 mmol, 30%, 96% Purity) as a pale-yellow solid. m/z 565.2 (M+H)⁺ (ES+). ¹H NMR (400 MHz, MeOD) δ 8.41 (s, 1H), 8.24 (dt, J=7.9, 1.4 Hz, 1H), 8.09 (t, J=1.8 Hz, 1H), 8.04 (s, 1H), 7.85-7.76 (m, 2H), 7.64-7.54 (m, 2H), 7.52-7.44 (m, 2H), 4.21 (t, J=7.7 Hz, 2H), 4.13-3.89 (m, 4H), 3.28 (s, 5H), 2.35 (p, J=7.8 Hz, 2H).

### Example 283: (1S,3R)-3-(((2-(4′-Fluoro-2′-(4-methyl-4H-1,2,4-triazol-3-yl)-[1,1′-biphenyl]-3-yl)-7-methoxybenzo[d]oxazol-5-yl)methyl)amino)-2,2-dimethylcyclobutan-1-ol (HF-1)

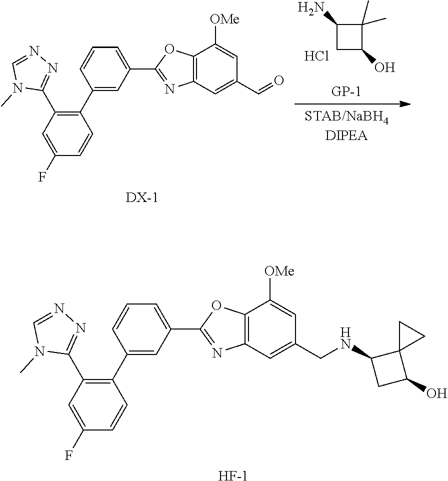

A mixture of intermediate (DX-1) (30 mg, 90% Wt, 1 Eq, 63 μmol), cis-3-amino-2,2-dimethylcyclobutanol, HCl (GP-1) (19 mg, 2.0 Eq, 0.13 mmol) and DIPEA (24 mg, 34 μL, 3.0 Eq, 0.19 mmol) in CHCl₃ (3 mL) was stirred at 40° C. for 90 min. NaBH(OAc)₃ (40 mg, 3.0 Eq, 0.19 mmol) was added and the reaction mixture was stirred at 40° C. overnight. NaBH₄ (24 mg, 10.0 Eq, 0.63 mmol) was added to the reaction mixture and stirring continued at 40° C. for an additional 2 h. The reaction mixture was quenched with water (0.5 mL) and allowed to stand at rt over the weekend. The reaction mixture was diluted with DCM (4 mL) and washed with sat. aq. NaHCO₃ (10 mL). The organic phase was separated and evaporated to afford the crude product. The crude product was purified by preparative HPLC (Waters, Basic (0.3% ammonia), Basic, Waters X-Bridge BEH C18 ODB Prep-C18, 5 μm, 30×100 mm column, 25-100% MeCN in water) to afford the title compound (HF-1) (5 mg, 9 μmol, 10%, 96% Purity) as a white solid. m/z 528.6 (M+H)⁺ (ES+). ¹H NMR (400 MHz, MeOD) δ 8.40 (s, 1H), 8.20 (dt, J=8.1, 1.3 Hz, 1H), 8.10 (t, J=1.8 Hz, 1H), 7.80 (dd, J=8.7, 5.4 Hz, 1H), 7.62-7.52 (m, 2H), 7.48 (dd, J=8.8, 2.7 Hz, 1H), 7.43-7.36 (m, 1H), 7.32 (d, J=1.3 Hz, 1H), 7.10 (d, J=1.3 Hz, 1H), 4.10 (s, 3H), 3.85 (q, J=13.1 Hz, 2H), 3.56 (dd, J=8.6, 7.2 Hz, 1H), 3.22 (s, 3H), 2.65-2.57 (m, 1H), 2.51-2.40 (m, 1H), 1.70-1.58 (m, 1H), 1.17 (s, 3H), 1.08 (s, 3H). Two exchangeable protons not observed.

### Example 284: (1S,2R)-2-[({2-[5-Cyclopropyl-4′-fluoro-2′-(4-methyl-1,2,4-triazol-3-yl)-[1,1′-biphenyl]-3-yl]-7-(trifluoromethyl)-1,3-benzoxazol-5-yl}methyl)amino]cyclopentan-1-ol (ACZ-8)

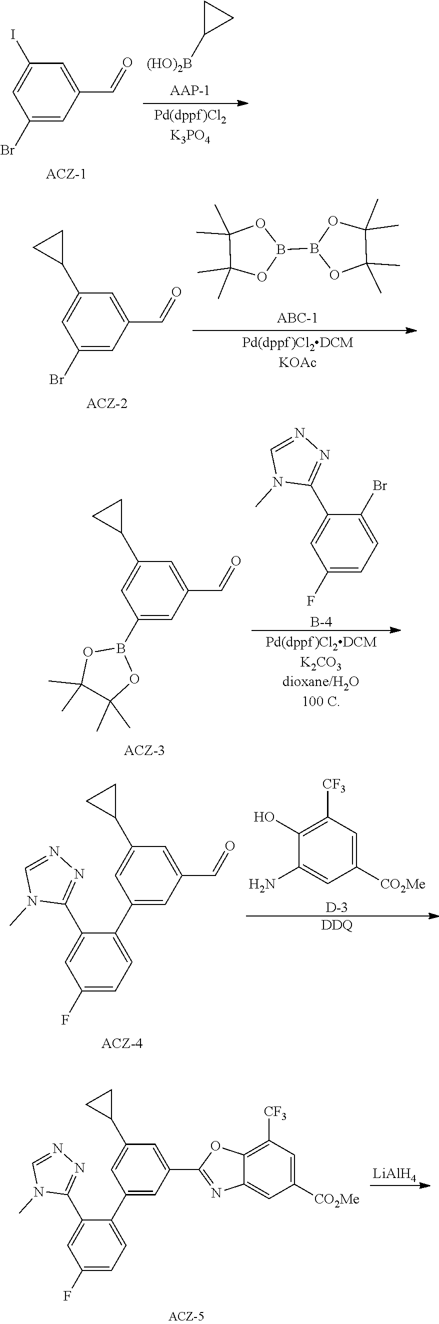

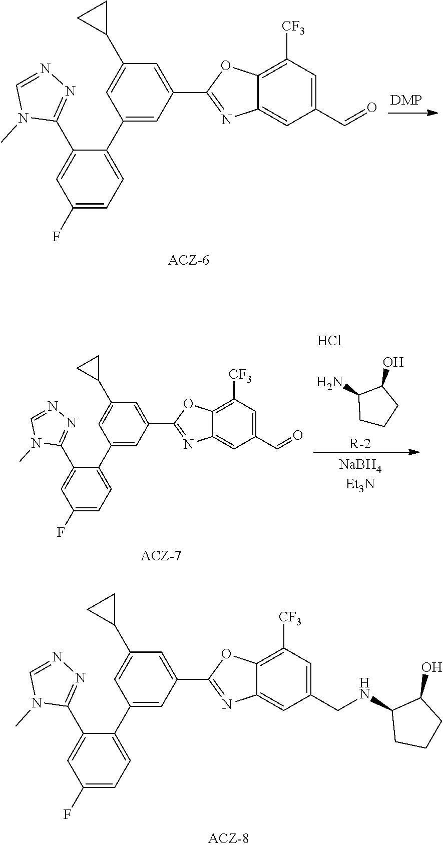

Step 1: 3-Bromo-5-cyclopropylbenzaldehyde (ACZ-2). To a stirred solution of 3-bromo-5-iodobenzaldehyde (ACZ-1) (1.00 g, 1 Eq, 3.21 mmol) and cyclopropylboronic acid (AAP-1) (280 mg, 1 Eq, 3.21 mmol) in dioxane (10 mL) was added K₃PO₄ (1.37 g, 2 Eq, 6.43 mmol) at rt under nitrogen atmosphere. Then Pd(dppf)Cl₂·DCM (240 mg, 0.1 Eq, 0.32 mmol) was added at rt under nitrogen atmosphere. The resulting mixture was stirred for overnight at 100° C. under nitrogen atmosphere. The mixture was cooled to rt. The resulting mixture was diluted with water and extracted with EtOAc (3×100 mL). The combined organic layers were washed with brine (2×100 mL), dried (Na₂SO₄). After filtration, the filtrate was concentrated in vacuo. The crude product was purified by reverse flash column chromatography with the following conditions: Column, C18; mobile phase, water (0.1% FA) and MeCN (15% MeCN up to 50% in 10 min); Detector, UV 254/220 nm. The product-containing fractions were combined and concentrated in vacuo. This resulted in the sub-title compound (ACZ-2) (430 mg, 1.78 mmol, 59%, 93% Purity) as a yellow oil. m/z 225.0/227.0 (M+H)⁺ (ES+).

Step 2: 3-Cyclopropyl-5-(4,4,5,5-tetramethyl-1,3,2-dioxaborolan-2-yl)benzaldehyde (ACZ-3). To a stirred solution of the product from step 1 above (ACZ-2) (750 mg, 1 Eq, 3.33 mmol) and bis(pinacolato)diboron (ABC-1) (1.27 g, 1.5 Eq, 4.99 mmol) in dioxane (10 mL) was added KOAc (654 mg, 2 Eq, 6.66 mmol) at rt under nitrogen atmosphere. Then Pd(dppf)Cl₂·DCM (244 mg, 0.1 Eq, 0.33 mmol) was added at rt under nitrogen atmosphere. The resulting mixture was stirred for 2 h at 100° C. under nitrogen atmosphere. The mixture was cooled to rt. The resulting mixture was diluted with water and extracted with EtOAc (3×80 mL). The combined organic layers were washed with brine (2×80 mL), dried (Na₂SO₄). After filtration, the filtrate was concentrated in vacuo. The crude product was purified by reverse flash column chromatography with the following conditions: Column, C18; mobile phase, water (0.1% FA) and MeCN (30% MeCN up to 60% in 10 min); Detector, UV 254/220 nm. The product-containing fractions were combined and concentrated in vacuo. This resulted in the sub-title compound (ACZ-3) (600 mg, 2.20 mmol, 66%, 90% Purity) as a yellow oil. m/z 273.2 (M+H)⁺ (ES+).

Step 3: 5-Cyclopropyl-4′-fluoro-2′-(4-methyl-1,2,4-triazol-3-yl)-[1,1′-biphenyl]-3-carbaldehyde (ACZ-4). To a stirred solution of the product from step 2 above (ACZ-3) (450 mg, 1 Eq, 1.65 mmol) and intermediate (B-4) (423 mg, 1 Eq, 1.65 mmol) in dioxane (10 mL) and water (2 mL) was added K₂CO₃ (457 mg, 2 Eq, 3.30 mmol) at rt under nitrogen atmosphere. Then Pd(dppf)Cl₂·DCM (121 mg, 0.1 Eq, 0.16 mmol) was added at rt under nitrogen atmosphere. The resulting mixture was stirred for 2 h at 100° C. under nitrogen atmosphere. The mixture was cooled to rt. The resulting mixture was diluted with water and extracted with EtOAc (3×80 mL). The combined organic layers were washed with brine (2×80 mL), dried (Na₂SO₄). After filtration, the filtrate was concentrated in vacuo. The residue was purified by Prep-TLC with EtOAc/petroleum ether (1/1). The product-containing fractions were combined and concentrated in vacuo. This resulted in the sub-title compound (ACZ-4) (400 mg, 1.24 mmol, 75%, 88% Purity) as a yellow solid. m/z 322.1 (M+H)⁺ (ES+).

Step 4: Methyl 2-[5-cyclopropyl-4′-fluoro-2′-(4-methyl-1,2,4-triazol-3-yl)-[1,1′-biphenyl]-3-yl]-7-(trifluoromethyl)-1,3-benzoxazole-5-carboxylate (ACZ-5). To a stirred solution of the product from step 3 above (ACZ-4) (280 mg, 1 Eq, 0.87 mmol) and intermediate (D-3) (246 mg, 1.2 Eq, 1.05 mmol) in DCM (10 mL) at rt. The resulting mixture was stirred for 1 h at 60° C. The mixture was allowed to cool down to rt. To the above mixture was added DDQ (396 mg, 2 Eq, 1.74 mmol) at rt. The resulting mixture was stirred for 2 h at rt. The resulting mixture was diluted with water and extracted with EtOAc (3×50 mL). The combined organic layers were washed with brine (2×50 mL), dried (Na₂SO₄). After filtration, the filtrate was concentrated in vacuo. The residue was purified by Prep-TLC with EtOAc/petroleum ether (1/1). The product-containing fractions were combined and concentrated in vacuo. This resulted in the sub-title compound (ACZ-5) (205 mg, 0.38 mmol, 44%, 92% Purity) as a yellow oil. m/z 537.2 (M+H)⁺ (ES+).

Step 5: {2-[5-Cyclopropyl-4′-fluoro-2′-(4-methyl-1,2,4-triazol-3-yl)-[1,1′-biphenyl]-3-yl]-7-(trifluoromethyl)-1,3-benzoxazol-5-yl}MeOH (ACZ-6). To a stirred solution of the product from step 4 above (ACZ-5) (100 mg, 1 Eq, 0.18 mmol) in THF (3 mL) was added LiAlH₄ (21 mg, 3 Eq, 0.55 mmol) at 0° C. under nitrogen atmosphere. The resulting mixture was stirred for 2 h at 0° C. under nitrogen atmosphere. The reaction was then quenched by the addition of 2 mL of ice water at 0° C. The resulting mixture was diluted with water and extracted with EtOAc (3×20 mL). The combined organic layers were washed with brine (2×20 mL), dried (Na₂SO₄). After filtration, the filtrate was concentrated in vacuo. The residue was purified by Prep-TLC with EtOAc/petroleum ether (1/1). The product-containing fractions were combined and concentrated in vacuo. This resulted in the sub-title compound (ACZ-6) (44 mg, 86 μmol, 46%, 90% Purity) as a white solid. m/z 509.2 (M+H)⁺ (ES+).

Step 6: 2-[5-Cyclopropyl-4′-fluoro-2′-(4-methyl-1,2,4-triazol-3-yl)-[1,1′-biphenyl]-3-yl]-7-(trifluoromethyl)-1,3-benzoxazole-5-carbaldehyde (ACZ-7). To a solution of the product from step 5 above (ACZ-6) (40 mg, 1 Eq, 79 μmol) in DCM (3 mL) was added Dess-Martin periodinane (43 mg, 1.3 Eq, 0.10 mmol) at rt. The resulting mixture was stirred for 2 h at rt. The resulting mixture was filtered the filter cake was washed with EtOAc (3×5 mL). The filtrate was concentrated in vacuo. This resulted in the sub-title compound (ACZ-7) (30 mg, 59 μmol, 75%, 92% Purity) as a white solid. m/z 507.1 (M+H)⁺ (ES+).

Step 7: (1S,2R)-2-[({2-[5-Cyclopropyl-4′-fluoro-2′-(4-methyl-1,2,4-triazol-3-yl)-[1,1′-biphenyl]-3-yl]-7-(trifluoromethyl)-1,3-benzoxazol-5-yl}methyl)amino]cyclopentan-1-ol (ACZ-8). To a stirred solution of the product from step 6 above (ACZ-7) (50 mg, 1 Eq, 90 μmol) and (1S,2R)-2-aminocyclopentan-1-ol (R-2) (11 mg, 1.1 Eq, 0.10 mmol) in DCM (5 mL) was added Et₃N (50 mg, 5 Eq, 0.49 mmol) at rt. The resulting mixture was stirred for 2 h at rt. To the above mixture was added NaBH₄ (37 mg, 10 Eq, 0.99 mmol) at 0° C. The resulting mixture was stirred for overnight at rt. The resulting mixture was diluted with water and extracted with DCM (3×20 mL). The combined organic layers were washed with brine (2×20 mL), dried (Na₂SO₄). After filtration, the filtrate was concentrated in vacuo. The crude product was purified by Prep-HPLC with the following conditions: Column: XBridge Prep OBD C18 Column, 19×250 mm, 5 μm; Mobile Phase A: water (0.1% NH₄HCO₃) Mobile Phase B: MeCN; Flow rate: 25 mL/min; Gradient: 30 B to 62 B in 9 min; Detector, UV 254/210 nm; RT: 8.15. The product-containing fractions were combined and evaporated partially in vacuo and lyophilized overnight to afford the title compound (ACZ-8) (9.8 mg, 16 μmol, 12.5%, 99% Purity) as a white solid. m/z 592.2 (M+H)⁺ (ES+). ¹H NMR (400 MHz, DMSO-d6) δ 8.44 (s, 1H), 8.33 (s, 1H), 8.08 (s, 1H), 7.92 (t, J=1.7 Hz, 1H), 7.83-7.76 (m, 2H), 7.73 (t, J=1.7 Hz, 1H), 7.59 (m, 1H), 7.53 (m, 1H), 6.86 (t, J=1.7 Hz, 1H), 4.00-3.93 (m, 3H), 3.15 (s, 3H), 2.84-2.74 (m, 1H), 2.14-1.99 (m, 1H), 1.77-1.52 (m, 4H), 1.42 (d, J=5.9 Hz, 2H), 1.03-0.94 (m, 2H), 0.63-0.54 (m, 2H).

### Example 285: (1S,2R)-2-{[(2-{6-Cyclopropyl-4-[4-fluoro-2-(4-methyl-1,2,4-triazol-3-yl)phenyl]pyridin-2-yl}-7-(trifluoromethyl)-1,3-benzoxazol-5-yl)methyl]amino}cyclopentan-1-ol (ADA-6)

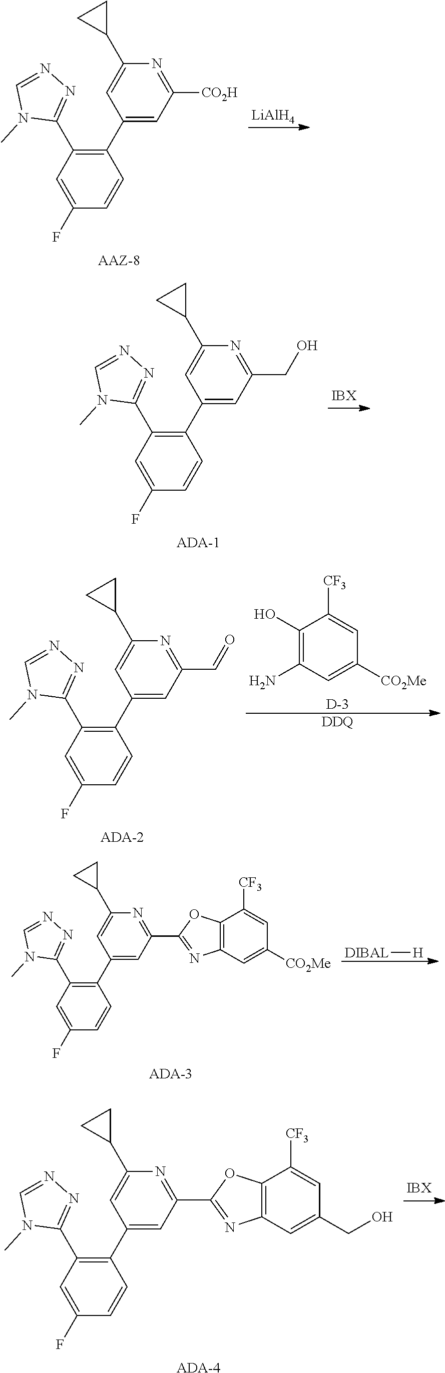

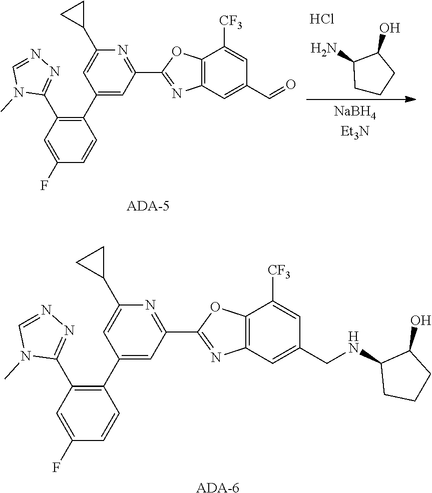 [SMILES: Cn1cnnc1-c1cc(F)ccc1-c1cc(-c2nc3cc(C=O)cc(C(F)(F)F)c3o2)nc(C2CC2)c1 ; Cl ; N[C@@H]1CCC[C@@H]1O ; "ADA-5" ; Cn1cnnc1-c1cc(F)ccc1-c1cc(-c2nc3cc(CN[C@@H]4CCC[C@@H]4O)cc(C(F)(F)F)c3o2)nc(C2CC2)c1 ; "ADA-6"]

Step 1: {6-Cyclopropyl-4-[4-fluoro-2-(4-methyl-1,2,4-triazol-3-yl)phenyl]pyridin-2-yl}MeOH (ADA-1). To a stirred solution of intermediate (AAZ-8) (200 mg, 1 Eq, 0.59 mmol) in THF (10 mL) was added LiAlH₄ in THF (1.77 mL, 1.0 M, 3 Eq, 1.77 mmol) dropwise at 0° C. under nitrogen atmosphere. The resulting mixture was stirred for 30 min at 0° C. under nitrogen atmosphere. The reaction was then quenched by the addition of 3 mL of ice water at 0° C. The mixture was concentrated in vacuo. The crude product was purified by reverse flash column chromatography with the following conditions: Column, C18; mobile phase, water (0.1% NH₄HCO₃) and MeCN (30% MeCN up to 60% in 10 min); Detector, UV 254/220 nm. The product-containing fractions were combined and concentrated in vacuo. This resulted in the sub-title compound (ADA-1) (80 mg, 0.25 mmol, 42%, 92% Purity) as a yellow solid. m/z 325.1 (M+H)⁺ (ES+).

Step 2: 6-Cyclopropyl-4-[4-fluoro-2-(4-methyl-1,2,4-triazol-3-yl)phenyl]pyridine-2-carbaldehyde (ADA-2). To a stirred solution of the product from step 1 above (ADA-1) (80 mg, 1 Eq, 0.25 mmol) in EtOAc (10 mL) was added IBX (104 mg, 1.5 Eq, 0.37 mmol) at rt. The resulting mixture was stirred for 2 h at 70° C. The resulting mixture was cooled to rt and concentrated in vacuo. The resulting mixture was filtered the filter cake was washed with EtOAc (3×5 mL). The filtrate was concentrated in vacuo. This resulted in the sub-title compound (ADA-2) (70 mg, 0.22 mmol, 88%, 88% Purity) as a yellow solid. m/z 323.1 (M+H)⁺ (ES+).

Step 3: Methyl 2-{6-cyclopropyl-4-[4-fluoro-2-(4-methyl-1,2,4-triazol-3-yl)phenyl]pyridin-2-yl}-7-(trifluoromethyl)-1,3-benzoxazole-5-carboxylate (ADA-3). To a stirred solution of the product from step 2 above (ADA-2) (60 mg, 1 Eq, 0.19 mmol) in DCM (5 mL) was added intermediate (D-3) (57 mg, 1.3 Eq, 0.24 mmol) at rt. The resulting mixture was stirred for 1 h at 60° C. The mixture was allowed to cool down to rt. To the above mixture was added DDQ (85 mg, 2 Eq, 0.37 mmol) at rt. The resulting mixture was stirred for 2 h at rt. The mixture was diluted with water and extracted with EtOAc (3×20 mL). The combined organic layers were washed with brine (2×20 mL), dried (Na₂SO₄). After filtration, the filtrate was concentrated in vacuo. The crude product was purified by reverse flash column chromatography with the following conditions: Column, C18; mobile phase, water (0.1% NH₄HCO₃) and MeCN (60% MeCN up to 80% in 10 min); Detector, UV 254/220 nm. The product-containing fractions were combined and concentrated in vacuo. This resulted in the sub-title compound (ADA-3) (40 mg, 0.07 mmol, 40%, 92% Purity) as a yellow solid. m/z 538.1 (M+H)⁺ (ES+).

Step 4: (2-{6-Cyclopropyl-4-[4-fluoro-2-(4-methyl-1,2,4-triazol-3-yl)phenyl]pyridin-2-yl}-7-(trifluoromethyl)-1,3-benzoxazol-5-yl)MeOH (ADA-4). To a stirred solution of the product from step 3 above (ADA-3) (40 mg, 1 Eq, 0.08 mmol) in THF (10 mL) was added DIBAL-H in THF (0.2 mL, 1.1 M, 3 Eq, 0.24 mmol) dropwise at 0° C. under nitrogen atmosphere. The resulting mixture was stirred for 1 h at 0° C. under nitrogen atmosphere. The reaction was then quenched by the addition of 2 mL of ice water at 0° C. The resulting mixture was diluted with water and extracted with DCM/MeOH=10:1 (3×20 mL). The combined organic layers were washed with brine (2×20 mL), dried (Na₂SO₄). After filtration, the filtrate was concentrated in vacuo. This resulted in the sub-title compound (ADA-4) (25 mg, 0.05 mmol, 66%, 90% Purity) as a yellow solid. m/z 510.2 (M+H)⁺ (ES+).

Step 5: 2-{6-Cyclopropyl-4-[4-fluoro-2-(4-methyl-1,2,4-triazol-3-yl)phenyl]pyridin-2-yl}-7-(trifluoromethyl)-1,3-benzoxazole-5-carbaldehyde (ADA-5). To a stirred solution of the product from step 4 above (ADA-4) (25 mg, 1 Eq, 0.05 mmol) in EtOAc (3 mL) was added IBX (21 mg, 1.5 Eq, 0.08 mmol) at rt. The resulting mixture was stirred for 1 h at 70° C. The mixture was allowed to cool down to rt. The resulting mixture was filtered the filter cake was washed with EtOAc (3×3 mL). The filtrate was concentrated in vacuo. This resulted in the sub-title compound (ADA-5) (20 mg, 0.04 mmol, 79%, 85% Purity) as a yellow solid. m/z 508.1 (M+H)⁺ (ES+).

Step 6: (1S,2R)-2-{[(2-{6-Cyclopropyl-4-[4-fluoro-2-(4-methyl-1,2,4-triazol-3-yl)phenyl]pyridin-2-yl}-7-(trifluoromethyl)-1,3-benzoxazol-5-yl)methyl]amino}cyclopentan-1-ol (ADA-6). To a stirred solution of the product from step 5 above (ADA-5) (20 mg, 1 Eq, 0.04 mmol) and (1S,2R)-2-aminocyclopentan-1-ol, HCl (R-2) (54 mg, 10 Eq, 0.39 mmol) in MeOH (3 mL) were added Et₃N (5 mg, 3 Eq, 0.12 mmol) at rt. The resulting mixture was stirred for 2 h at 60° C. To the above mixture was added NaBH₄ (9 mg, 3 Eq, 0.24 mmol) at rt. The resulting mixture was stirred for overnight at rt. The reaction was then quenched by the addition of 2 mL of MeOH at 0° C. The mixture was concentrated in vacuo. The residue was purified by Prep-TLC with DCM/MeOH (10/1). The crude product was purified by Prep-HPLC with the following conditions (Column: XBridge Prep OBD C18 Column, 30*150 mm, 5 μm; Mobile Phase A: water (0.1% NH₄HCO₃), Mobile Phase B: MeCN; Flow rate: 60 mL/min; Gradient: 30% B to 70% B in 9 min; Wave Length: 254/220 nm; RT: 8.18) to afford the title compound (ADA-6) (4.1 mg, 6.9 μmol, 18%, 98% Purity) as a white solid. m/z 593.2 (M+H)⁺ (ES+). ¹H NMR (400 MHz, MeOH-d4) δ 8.46 (s, 1H), 8.07 (s, 1H), 7.86-7.82 (m, 3H), 7.61-7.49 (m, 2H), 7.16 (d, J=1.6 Hz, 1H), 4.16 (s, 1H), 4.07-3.95 (m, 2H), 3.36 (s, 3H), 2.99-2.97 (m, 1H), 2.20-2.16 (m, 1H), 1.93-1.72 (m, 4H), 1.57-1.55 (m, 2H), 1.08-1.06 (m, 4H).

### Example 286: (1S,2R)-2-(((2-(4′-Chloro-2′-(4-methyl-4H-1,2,4-triazol-3-yl)-[1,1′-biphenyl]-3-yl)-7-(trifluoromethyl)-1H-benzo[d]imidazol-5-yl)methyl)amino)cyclopentan-1-ol (ADB-9)

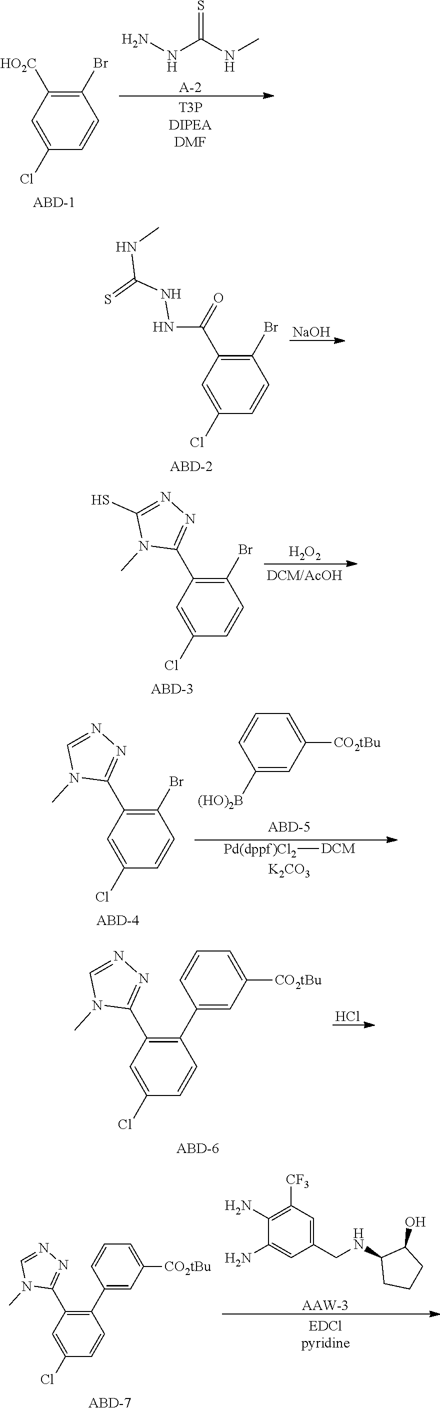

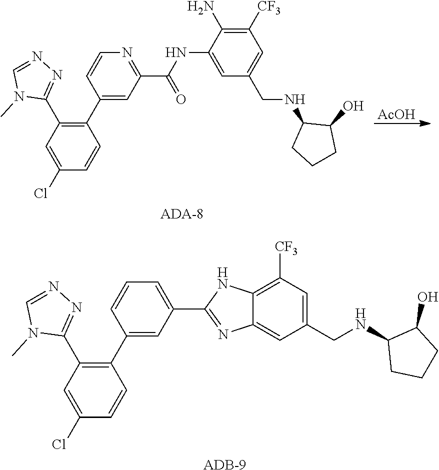 [SMILES: "AcOH" ; Cn1cnnc1-c1cc(Cl)ccc1-c1ccnc(C(=O)Nc2cc(CN[C@@H]3CCC[C@@H]3O)cc(C(F)(F)F)c2N)c1 ; "ADA-8" ; Cn1cnnc1-c1cc(Cl)ccc1-c1cccc(-c2nc3cc(CN[C@@H]4CCC[C@@H]4O)cc(C(F)(F)F)c3[nH]2)c1 ; "ADB-9"]

Step 1: 2-Bromo-5-chloro-N-[(methylcarbamothioyl)amino]benzamide (ADB-2). To a stirred mixture of 2-bromo-5-chlorobenzoic acid (ADB-1) (4.90 g, 1 eq, 20.8 mmol) and DIPEA (8.07 g, 3 eq, 62.4 mmol) in DMF (50 mL) was added 4-methyl-3-thiosemicarbazide (A-2) (2.63 g, 1.2 eq, 25.0 mmol) and T₃P in EtOAc (26.5 g, 50% Wt, 4 eq, 83.2 mmol) at 0° C. The resulting mixture was stirred for overnight at rt. The resulting mixture was concentrated in vacuo. The crude product was purified by reverse flash column chromatography with the following conditions: Column, C18; mobile phase, water (0.1% FA) and MeCN (0% MeCN up to 60% in 30 min); Detector, UV 254/220 nm to afford the sub-title compound (ADB-2) (4.5 g, 11.5 mmol, 67%, 82% Purity) as a yellow solid. m/z 321.9/323.9 (M+H)⁺ (ES+).

Step 2: 5-(2-Bromo-5-chlorophenyl)-4-methyl-1,2,4-triazole-3-thiol (ADB-3). To a stirred mixture of NaOH (2.79 g, 69.745 mmol, 5 eq) in H₂O (200 g) was added the product from step 1 above (ADB-2) (4.50 g, 1 eq, 14.0 mmol) at 0° C. The resulting mixture was stirred for 2 h at 60° C. The mixture was acidified to pH 4 with HCl (aq., 1 M) at 0° C. The resulting mixture was diluted with water and extracted with DCM (3×300 mL). The combined organic layers were washed with brine (2×300 mL), dried (Na₂SO₄). After filtration, the filtrate was concentrated in vacuo. This resulted in the sub-title compound (ADB-3) (4.3 g, 10.6 mmol, crude, 75% Purity) as a yellow solid. m/z 303.9/305.9 (M+H)⁺ (ES+).

Step 3: 3-(2-Bromo-5-chlorophenyl)-4-methyl-1,2,4-triazole (ADB-4). To a stirred mixture of the product from step 2 above (ADB-3) (4.3 g, 1 eq, 14.1 mmol) in DCM (50 mL) was added AcOH (1.70 g, 2 eq, 28.2 mmol) dropwise at 0° C. To the above mixture was added H₂O₂ (2.40 g, 30% Wt, 5 eq, 70.6 mmol) dropwise at 0° C. The resulting mixture was stirred for 2 h at rt. The mixture was acidified to pH 7 with saturated NaHCO₃ (aq., 1 M). The resulting mixture was diluted with water and extracted with DCM (3×300 mL). The combined organic layers were washed with brine (2×300 mL), dried (Na₂SO₄). After filtration, the filtrate was concentrated in vacuo. This resulted in the sub-title compound (ADB-4) (2.8 g, 8.30 mmol, 73%, 80% Purity) as a yellow solid. m/z 271.0/273.0 (M+H)⁺ (ES+).

Step 4: tert-Butyl 4′-chloro-2′-(4-methyl-4H-1,2,4-triazol-3-yl)-[1,1′-biphenyl]-3-carboxylate (ADB-6). To a stirred mixture of the product from step 3 above (ADB-4) (130 mg, 1 Eq, 0.48 mmol) and 3-(tert-butoxycarbonyl)phenylboronic acid (ADB-5) (106 mg, 1 Eq, 0.48 mmol) in dioxane (5 mL) and water (1 mL) was added K₂CO₃ (198 mg, 3 Eq, 1.43 mmol) at rt under nitrogen atmosphere. To the above mixture was added Pd(dppf)Cl₂·DCM (78 mg, 0.2 Eq, 95 μmol) at rt. The resulting mixture was stirred for overnight at 60° C. under nitrogen atmosphere. The mixture was cooled to rt and concentrated in vacuo. The residue was purified by Prep-TLC (DCM/MeOH 12:1). The resulting mixture was concentrated in vacuo to afford the sub-title compound (ADB-6) (52 mg, 0.14 mmol, 29%, 90% Purity) as a yellow solid. m/z 370.1/372.1 (M+H)⁺ (ES+).

Step 5: 4′-Chloro-2′-(4-methyl-4H-1,2,4-triazol-3-yl)-[1,1′-biphenyl]-3-carboxylic acid (ADB-7). To a stirred mixture of the product from step 4 above (ADB-6) (52 mg, 1 Eq, 0.14 mmol) in DCM (2 mL) was added HCl in dioxane (2.5 mg, 4 M, 0.5 Eq, 0.07 mmol) dropwise at rt. The resulting mixture was stirred for 1 h at rt. The resulting mixture was concentrated in vacuo. This resulted in the sub-title compound (ADB-7) (100 mg, 0.32 mmol, crude, 72% Purity) as a yellow solid. m/z 314.1/316.1 (M+H)⁺ (ES+).

Step 6: N-(2-Amino-5-((((1R,2S)-2-hydroxycyclopentyl)amino)methyl)-3-(trifluoromethyl)phenyl)-4′-chloro-2′-(4-methyl-4H-1,2,4-triazol-3-yl)-[1,1′-biphenyl]-3-carboxamide (ADB-8). To a stirred mixture of the product from step 5 above (ADB-7) and intermediate (AAW-3) (91 mg, 1.2 Eq, 0.31 mmol) in pyridine (10 mL) was added EDCI (101 mg, 2 Eq, 0.53 mmol) at 0° C. under nitrogen atmosphere. The resulting mixture was stirred for 2 h at 60° C. under nitrogen atmosphere. The resulting mixture was cooled to rt and concentrated in vacuo. The crude product was purified by reverse flash column chromatography with the following conditions: Column, C18; mobile phase, water (0.1% TFA) and MeCN (36% MeCN up to 48% in 10 min); Detector, UV 254/220 nm. The product-containing fractions were combined and concentrated in vacuo. This resulted in the sub-title compound (ADB-8) (42 mg, 72 μmol, 27%, 89% Purity) as a yellow solid. m/z 585.2/587.2 (M+H)⁺ (ES+).

Step 7: (1S,2R)-2-(((2-(4′-Chloro-2′-(4-methyl-4H-1,2,4-triazol-3-yl)-[1,1′-biphenyl]-3-yl)-7-(trifluoromethyl)-1H-benzo[d]imidazol-5-yl)methyl)amino)cyclopentan-1-ol (ADB-9). A mixture of the product from step 6 above (ADB-8) (35 mg, 1 Eq, 60 μmol) and AcOH (5 mL) was stirred for 2.5 h at 80° C. under nitrogen atmosphere. The mixture was cooled to rt and concentrated in vacuo. The crude product was purified by Prep-HPLC with the following conditions (Column: Sunfire prep C18 column, 30*150 mm, 5 μm; Mobile Phase A: water (0.1% FA), Mobile Phase B: MeCN; Flow rate: 60 mL/min; Gradient: 10% B to 28% B in 10 min; Wave Length: 254/220 nm; RT: 10.08) to afford the title compound (ADB-9) (13.9 mg, 24 μmol, 41%, 99% Purity) as a white solid. m/z 567.3/569.3 (M+H)⁺ (ES+). ¹H NMR (300 MHz, Methanol-d4) δ 8.36 (s, 1H), 8.18-8.08 (m, 2H), 7.99 (s, 1H), 7.83-7.67 (m, 4H), 7.50 (t, J=8.1 Hz, 1H), 7.26 (d, J=7.8 Hz, 1H), 4.42-4.25 (m, 3H), 3.45-3.36 (m, 1H), 3.21 (s, 3H), 2.12-1.60 (m, 6H).

### Example 287: Methyl 2-(3-((1s,3s)-3-methyl-1-(4-methyl-4H-1,2,4-triazol-3-yl)cyclobutyl)phenyl)-7-(trifluoromethyl)benzo[d]oxazole-5-carboxylate (ADC-3)

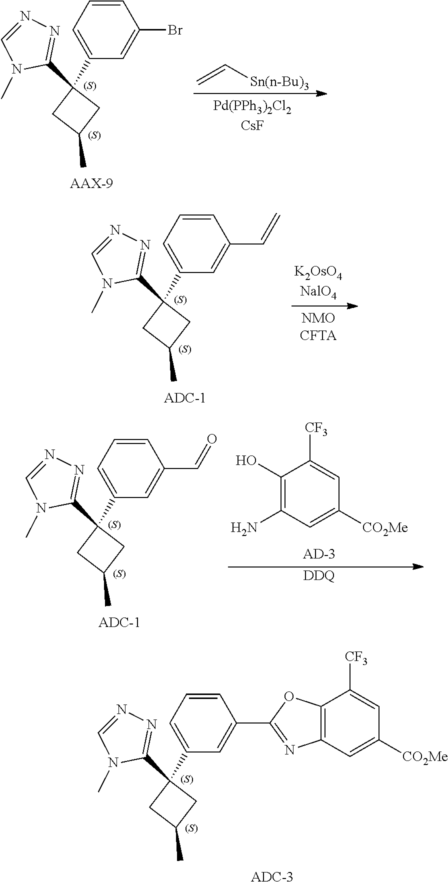

Step 1: 4-Methyl-3-[(1r,3s)-1-(3-ethenylphenyl)-3-methylcyclobutyl]-1,2,4-triazole (ADC-2). To a solution of intermediate (AAX-9) (500 mg, 1 Eq, 1.63 mmol) and tributyl(ethenyl)stannane (1.04 g, 2 Eq, 3.27 mmol) in 1,4-dioxane (10 mL) was added CsF (744 mg, 3 Eq, 4.90 mmol) at rt under nitrogen atmosphere. Then Pd(PPh₃)₂Cl₂ (229 mg, 0.2 Eq, 0.33 mmol) was added at rt under nitrogen atmosphere. The resulting mixture was stirred for 2 h at 100° C. under nitrogen atmosphere. The mixture was cooled to rt, diluted with water and extracted with EtOAc (3×80 mL). The combined organic layers were washed with brine (2×80 mL), dried (Na₂SO₄). After filtration, the filtrate was concentrated in vacuo. The residue was purified by Prep-TLC with DCM/MeOH (15/1) to afford the sub-title compound (ADC-1) (400 mg, 1.57 mmol, 97%, 92% Purity) as a yellow oil. m/z 254.2 (M+H)⁺ (ES+).

Step 2: 3-[(1r,3s)-3-Methyl-1-(4-methyl-1,2,4-triazol-3-yl)cyclobutyl]benzaldehyde (ADC-2). To a stirred solution of the product from step 1 above (ADC-1) (400 mg, 1 Eq, 1.58 mmol), citric acid (406 mg, 1.34 Eq, 2.12 mmol) and NMO (248 mg, 1.34 Eq, 2.12 mmol) in tBuOH (8 mL) and water (8 mL) were added K₂OsO₄·2H₂O (31 mg, 0.06 Eq, 0.09 mmol) at rt. After 2 h, to the above mixture was added NaIO₄ (676 mg, 2 Eq, 3.16 mmol) at rt. The resulting mixture was stirred for 1 h at rt. The crude product was purified by reverse flash column chromatography with the following conditions: Column, C18; mobile phase, water (0.1% NH₄HCO₃) and MeCN (30% MeCN up to 40% in 10 min); Detector, UV 254/220 nm. The product-containing fractions were combined and concentrated in vacuo. This resulted in the sub-title compound (ADC-2) (240 mg, 0.94 mmol, 60%, 90% Purity) as a yellow solid. m/z 256.1 (M+H)⁺ (ES+).

Step 3: Methyl 2-{3-[(1r,3s)-3-methyl-1-(4-methyl-1,2,4-triazol-3-yl)cyclobutyl]phenyl}-7-(trifluoromethyl)-1,3-benzoxazole-5-carboxylate (ADC-3). A solution of the product from step 2 above (ADC-2) (20 mg, 78 μmol, 1 Eq) and intermediate (D-3) (37 mg, 2 Eq, 0.16 mmol) in DCM (2 mL) was stirred for 1 h at 60° C. The mixture was allowed to cool down to rt. To the above mixture was added DDQ (26 mg, 2 Eq, 0.16 mmol) at rt. The resulting mixture was stirred for additional 2 h at rt. The mixture was diluted with water and extracted with EtOAc (3×20 mL). The combined organic layers were washed with brine (2×20 mL), dried (Na₂SO₄). After filtration, the filtrate was concentrated in vacuo. The crude product was purified by Prep-HPLC with the following conditions (Column: XBridge Prep OBD C18 Column, 30*150 mm, 5 μm; Mobile Phase A: water (0.1% NH₄HCO₃+0.1% NH₃·H₂O), Mobile Phase B: MeCN; Flow rate: 60 mL/min; Gradient: 48% B to 62% B in 8 min; Wave Length: 254/220 nm; RT: 7.68) to afford the title compound (ADC-3) (11.2 mg, 24 μmol, 31%, 99% Purity) as a white solid. m/z 471.3 (M+H)⁺ (ES+). ¹H NMR (300 MHz, MeOH-d4) δ 8.60 (s, 1H), 8.33-8.20 (m, 3H), 8.22-8.17 (m, 1H), 7.73-7.62 (m, 2H), 3.99 (d, J=1.2 Hz, 3H), 3.30 (s, 3H), 3.02-2.97 (m, 2H), 2.73-2.62 (m, 3H), 1.20-1.17 (m, 3H).

### Example 288: (2-(3-((1s,3s)-3-Methyl-1-(4-methyl-4H-1,2,4-triazol-3-yl)cyclobutyl)phenyl)-7-(trifluoromethyl)benzo[d]oxazol-5-yl)methanol (ADD-1)

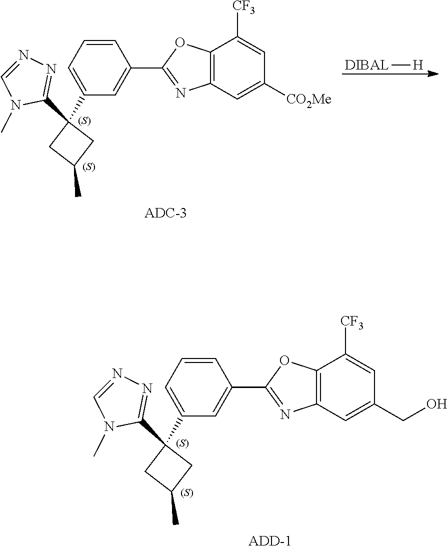

To a stirred solution of compound (ADC-3) (20 mg, 1 Eq, 37 μmol) in THF (2 mL) was added DIBAL-H in THF (0.39 mL, 1.1M, 3 Eq, 0.11 mmol) dropwise at 0° C. under nitrogen atmosphere. The resulting mixture was stirred for 1 h at 0° C. under nitrogen atmosphere. The reaction was then quenched by the addition of 5 mL of ice water at 0° C. The resulting mixture was diluted with water and extracted with DCM/MeOH=10:1 (3×20 mL). The combined organic layers were washed with brine (2×20 mL), dried (Na₂SO₄). After filtration, the filtrate was concentrated in vacuo. The crude product was purified by Prep-HPLC with the following conditions (Column: XBridge Prep OBD C18 Column, 30*150 mm, 5 μm; Mobile Phase A: water (0.1% NH₄HCO₃+0.1% NH₃·H₂O), Mobile Phase B: MeCN; Flow rate: 60 mL/min; Gradient: 40% B to 50% B in 9 min; Wave Length: 254/220 nm; RT: 6.30) to afford the title compound (ADD-1) (7.9 mg, 18 μmol, 48%, 99% Purity) as an off-white solid. m/z 443.1 (M+H)⁺ (ES+). ¹H NMR (300 MHz, MeOH-d4) δ 8.33-8.28 (m, 2H), 8.20-8.15 (m, 1H), 7.98 (s, 1H), 7.71-7.61 (m, 3H), 4.79 (s, 2H), 3.30 (s, 3H), 3.05-2.95 (m, 2H), 2.77-2.61 (m, 3H), 1.20-1.16 (m, 3H).

### Example 289: (1S,2R)-2-{[(2-{3-[(1r,3s)-3-Methyl-1-(4-methyl-1,2,4-triazol-3-yl)cyclobutyl]phenyl}-7-trifluoromethyl)-1,3-benzoxazol-5-yl)methyl]amino}cyclopentan-1-ol (ADE-2)

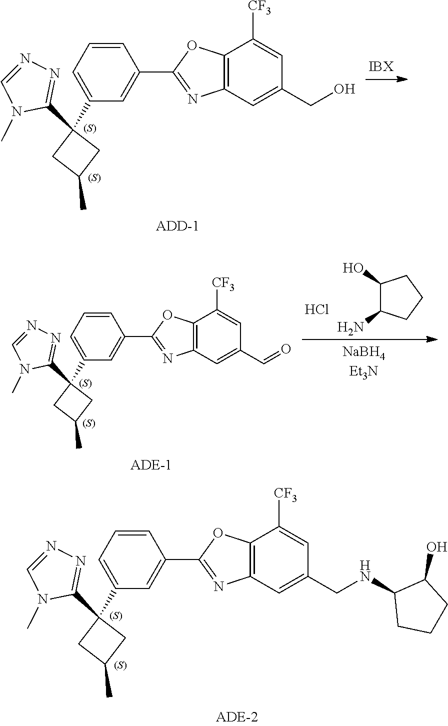

Step 1: 2-{3-[(1r,3s)-3-Methyl-1-(4-methyl-1,2,4-triazol-3-yl)cyclobutyl]phenyl}-7-(trifluoromethyl)-1,3-benzoxazole-5-carbaldehyde (ADE-1). To a stirred solution of compound (ADD-1) (180 mg, 1 Eq, 0.41 mmol) in EtOAc (18 mL) was added IBX (228 mg, 2 Eq, 0.81 mmol) at rt. The resulting mixture was stirred for 1 h at 70° C. The mixture was allowed to cool down to rt. The resulting mixture was filtered the filter cake was washed with EtOAc (3×5 mL). The filtrate was concentrated in vacuo. This resulted in the sub-title compound (ADE-1) (150 mg, 0.34 mmol, 89%, 92% Purity) as a yellow solid. m/z 441.1 (M+H)⁺ (ES+).

Step 2: (1S,2R)-2-{[(2-{3-[(1r,3s)-3-Methyl-1-(4-methyl-1,2,4-triazol-3-yl)cyclobutyl]phenyl}-7-(trifluoromethyl)-1,3-benzoxazol-5-yl)methyl]amino}cyclopentan-1-ol (ADE-2). To a stirred solution of the product from step 1 above (ADE-1) (30 mg, 1 Eq, 68 μmol) and (1S,2R)-2-aminocyclopentan-1-ol, HCl (R-2) (28 mg, 3 Eq, 0.20 mmol) in MeOH (3 mL) was added Et₃N (21 mg, 3 Eq, 0.20 mmol) at rt. The resulting mixture was stirred for 2 h at 60° C. To the above mixture was added NaBH₄ (8 mg, 3 Eq, 0.20 mmol) at rt. The resulting mixture was stirred for 2 h at rt. The reaction was then quenched by the addition of 2 mL of MeOH at 0° C. The mixture was concentrated in vacuo. The residue was purified by Prep-TLC with DCM/MeOH (10/1). The crude product was purified by Prep-HPLC with the following conditions (Column: XBridge Prep OBD C18 Column, 30*150 mm, 5 μm; Mobile Phase A: water (0.1% NH₄HCO₃+0.1% NH₃·H₂O), Mobile Phase B: MeCN; Flow rate: 60 mL/min; Gradient: 40% B to 50% B in 9 min; Wave Length: 254/220 nm; RT: 8.35) to afford the title compound (ADE-2) (7.0 mg, 13 μmol, 20%, 98% Purity) as an off-white solid. m/z 526.4 (M+H)⁺ (ES+). ¹H NMR (300 MHz, MeOH-d4) δ8.33-8.28 (m, 2H), 8.19-8.16 (m, 1H), 8.02 (s, 1H), 7.76 (s, 1H), 7.67-7.62 (m, 2H), 4.16 (s, 1H), 4.09-3.90 (m, 2H), 3.27 (s, 3H), 3.03-2.92 (m, 3H), 2.75-2.62 (m, 3H), 1.94-1.71 (m, 4H), 1.63-1.49 (m, 2H), 1.20-1.17 (m, 3H).

### Example 290: Methyl 2-[4′-fluoro-2′-(4-methyl-1,2,4-triazol-3-yl)-[1,1′-biphenyl]-3-yl]-7-(trifluoromethyl)-1,3-benzoxazole-5-carboxylate (X-1)

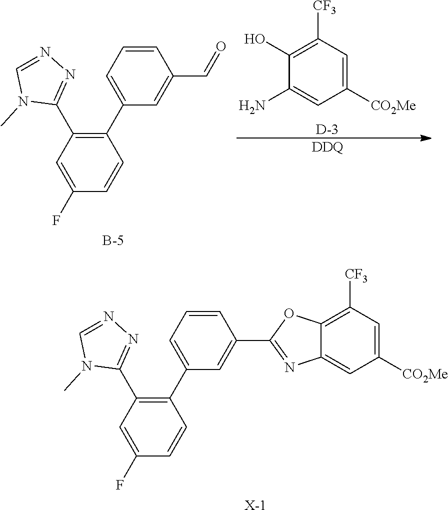

A solution of intermediate (B-5) (100 mg, 1 Eq, 0.36 mmol) and intermediate (D-3) (84 mg, 1 Eq, 0.36 mmol) in DCM (10 mL) was stirred for 3 h at 60° C. To the above mixture was added DDQ (161 mg, 2 Eq, 0.71 mmol) at rt. The resulting mixture was stirred for additional 2 h at rt. The resulting mixture was diluted with water and extracted with EtOAc (3×50 mL). The combined organic layers were washed with brine (2×50 mL), dried (Na₂SO₄). After filtration, the filtrate was concentrated in vacuo. The residue was purified by Prep-TLC with DCM/MeOH (5/1). The crude product was purified by Prep-HPLC with the following conditions (Column: XBridge Prep OBD C18 Column, 30*150 mm, 5 μm; Mobile Phase A: water (0.1% NH₄HCO₃), Mobile Phase B: MeCN; Flow rate: 60 mL/min; Gradient: 48% B to 57% B in 9 min; Wave Length: 254/220 nm) to afford the title compound (X-1) (14.0 mg, 28 μmol, 7.9%, 99% Purity) as a white solid. m/z 496.9 (M+H)⁺ (ES+). ¹H NMR (400 MHz, MeOH-d4) δ 8.62-8.58 (m, 1H), 8.41-8.31 (m, 2H), 8.26-8.21 (m, 1H), 8.08 (t, J=1.6 Hz, 1H), 7.81-7.75 (m, 1H), 7.63-7.51 (m, 2H), 7.50-7.44 (m, 2H), 4.00 (s, 3H), 3.25 (s, 3H).

### Example 291: (1R,2R)-2-[({2-[4′-Fluoro-2′-(4-methyl-1,2,4-triazol-3-yl)-[1,1′-biphenyl]-3-yl]-7-(trifluoromethyl)-1,3-benzoxazol-5-yl}methyl)amino]cyclobutan-1-ol (ADF-2)

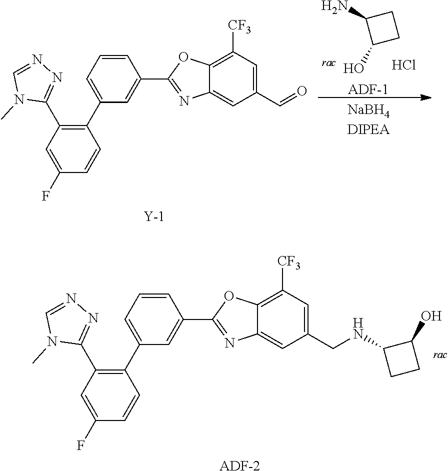

To a stirred solution of intermediate (Y-1) (130 mg, 1 Eq, 0.28 mmol) and (1S,2S)-2-aminocyclobutan-1-ol, HCl (ADF-1) (52 mg, 1.5 Eq, 0.42 mmol) in DCM (10 mL) was added DIPEA (108 mg, 3 Eq, 0.84 mmol) at rt. To the above mixture was added NaBH₄ (22 mg, 2 Eq, 0.55 mmol) over 1 h at 0° C. The resulting mixture was stirred for additional 30 mins at rt. The reaction was quenched with MeOH (1 mL) at 0° C. and concentrated in vacuo. The residue was purified by Prep-TLC with DCM/MeOH (6/1). The crude product was purified by Prep-HPLC with the following conditions (Column: XBridge Prep OBD C18 Column, 30*150 mm, 5 μm; Mobile Phase A: water (0.1% NH₄HCO₃), Mobile Phase B: MeCN; Flow rate: 60 mL/min; Gradient: 33% B to 44% B in 9 min; Wave Length: 254/220 nm) to afford the title compound (ADF-2) (2.3 mg, 4.3 μmol, 1.5%, 98% Purity) as a white solid. m/z 538.0 (M+H)⁺ (ES+). ¹H NMR (400 MHz, DMSO-d6) δ 8.42 (s, 1H), 8.18-8.10 (m, 1H), 8.05 (s, 1H), 7.91 (d, J=1.9 Hz, 1H), 7.82-7.74 (m, 2H), 7.65-7.50 (m, 3H), 7.41-7.35 (m, 1H), 5.05 (d, J=6.8 Hz, 1H), 3.90 (s, 2H), 3.79-3.68 (m, 1H), 3.21 (s, 3H), 2.93-2.83 (m, 1H), 1.94-1.84 (m, 1H), 1.80-1.69 (m, 1H), 1.37-1.20 (m, 1H), 1.11-1.00 (m, 1H).

### Example 292: (1S,2S)-2-[({2-[4′-Fluoro-2′-(4-methyl-1,2,4-triazol-3-yl)-[1,1′-biphenyl]-3-yl]-7-(trifluoromethyl)-1,3-benzoxazol-5-yl}methyl)amino]cyclobutan-1-ol (ADG-1)

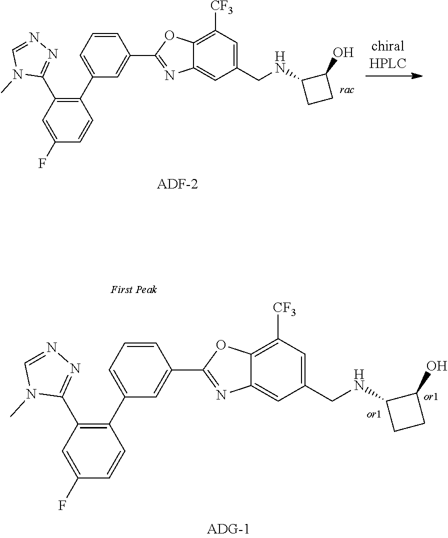 [SMILES: Cn1cnnc1-c1cc(F)ccc1-c1cccc(-c2nc3cc(CN[C@H]4CC[C@@H]4O)cc(C(F)(F)F)c3o2)c1 ; "ADF-2" ; Cn1cnnc1-c1cc(F)ccc1-c1cccc(-c2nc3cc(CN[C@H]4CC[C@@H]4O)cc(C(F)(F)F)c3o2)c1]

Racemic (ADF-2) (25 mg, 1 Eq, 47 μmol) was separated by chiral Prep-HPLC with the following conditions (Column: CHIRAL ART Cellulose-SC, 2*25 cm, 5 μm; Mobile Phase A: Hex:DCM=3:1 (0.5% 2M NH₃-MeOH)-HPLC, Mobile Phase B: EtOH-HPLC; Flow rate: 20 mL/min; Gradient: 30% B to 30% B in 18 min; Wave Length: 220/254 nm; RT1: 10.60; RT2: 14.50) to afford the title compound (ADG-1) (16.6 mg, 31 μmol, 66%, 99% Purity) as a white solid. m/z 538.3 (M+H)⁺ (ES+). ¹H NMR (400 MHz, MeOH-d4) δ 8.37 (s, 1H), 8.25-8.18 (m, 1H), 8.10-7.97 (m, 2H), 7.83-7.72 (m, 2H), 7.63-7.52 (m, 2H), 7.49-7.39 (m, 2H), 4.07-3.89 (m, 3H), 3.24 (s, 3H), 3.15-3.03 (m, 1H), 2.15-2.03 (m, 1H), 2.01-1.87 (m, 1H), 1.56-1.42 (m, 1H), 1.34-1.17 (m, 1H). Column: CHIRALPAK IC-3, 4.6*50 mm, 3 μm; Mobile Phase A: (Hex:DCM=3:1) (0.1% DEA):EtOH=50:50; Flow rate: 1 mL/min; RT: 2.183.

### Example 293: (1R,2R)-2-[({2-[4′-Fluoro-2′-(4-methyl-1,2,4-triazol-3-yl)-[1,1′-biphenyl]-3-yl]-7-(trifluoromethyl)-1,3-benzoxazol-5-yl}methyl)amino]cyclobutan-1-ol (ADH-1)

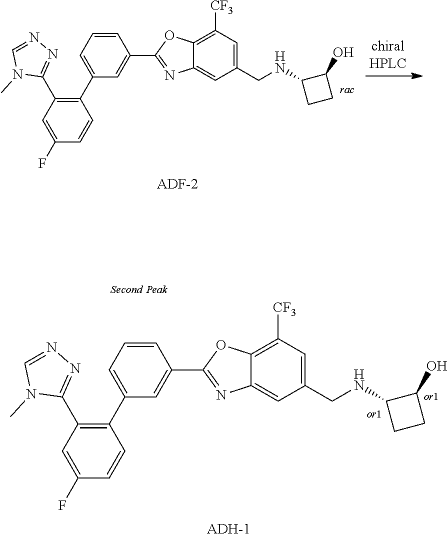 [SMILES: Cn1cnnc1-c1cc(F)ccc1-c1cccc(-c2nc3cc(CN[C@H]4CC[C@@H]4O)cc(C(F)(F)F)c3o2)c1 ; "ADF-2" ; Cn1cnnc1-c1cc(F)ccc1-c1cccc(-c2nc3cc(CN[C@H]4CC[C@@H]4O)cc(C(F)(F)F)c3o2)c1 ; "ADH-1"]

Racemic (ADF-2) (25 mg, 1 Eq, 47 μmol) was separated by chiral Prep-HPLC with the following conditions (Column: CHIRAL ART Cellulose-SC, 2*25 cm, 5 μm; Mobile Phase A: Hex:DCM=3:1 (0.5% 2M NH₃-MeOH)-HPLC, Mobile Phase B: EtOH-HPLC; Flow rate: 20 mL/min; Gradient: 30% B to 30% B in 18 min; Wave Length: 220/254 nm; RT1: 10.60; RT2: 14.50) to afford the title compound (ADH-1) (17.6 mg, 33 μmol, 69%, 97% Purity) as a white solid. m/z 538.3 (M+H)⁺ (ES+). ¹H NMR (400 MHz, MeOH-d4) δ 8.37 (s, 1H), 8.25-8.18 (m, 1H), 8.10-7.97 (m, 2H), 7.83-7.72 (m, 2H), 7.63-7.52 (m, 2H), 7.49-7.39 (m, 2H), 4.07-3.89 (m, 3H), 3.24 (s, 3H), 3.15-3.03 (m, 1H), 2.15-2.03 (m, 1H), 2.01-1.87 (m, 1H), 1.56-1.42 (m, 1H), 1.34-1.17 (m, 1H). Column: CHIRALPAK IC-3, 4.6*50 mm, 3 μm; Mobile Phase A: (Hex:DCM=3:1)(0.1% DEA):EtOH=50:50; Flow rate: 1 mL/min; RT: 2.814.

### Example 294: (1S,2R)-2-[({2-[4′-Chloro-2′-(4-methyl-1,2,4-triazol-3-yl)-[1,1′-biphenyl]-3-yl]-7-(trifluoromethyl)-1,3-benzoxazol-5-yl}methyl)amino]cyclopentan-1-ol (ADI-3)

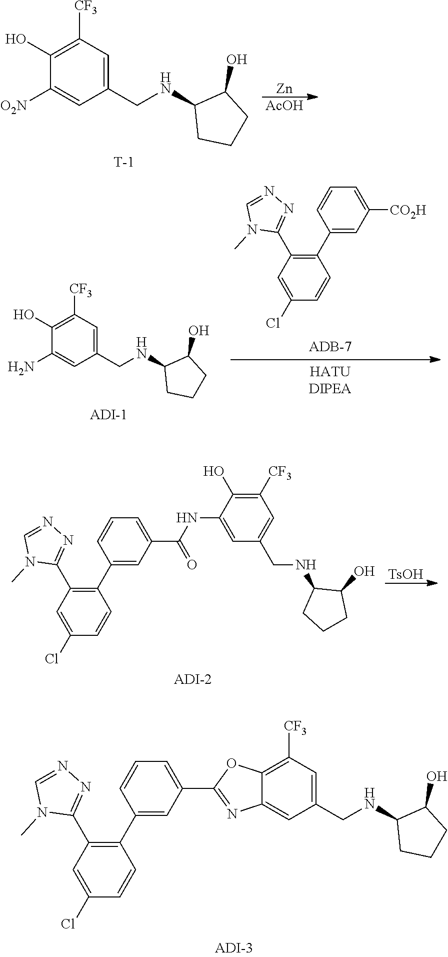

Step 1: 2-Amino-4-({[(1R,2S)-2-hydroxycyclopentyl]amino}methyl)-6-(trifluoromethyl)phenol (ADI-1). To a stirred mixture of intermediate (T-1) (160 mg, 1 Eq, 0.50 mmol) in AcOH (5 mL) was added Zn powder (163 mg, 5 Eq, 2.50 mmol) at rt. The resulting mixture was stirred for 2 h at rt. The resulting mixture was diluted with water. The resulting mixture was extracted with DCM (3×15 mL). The combined organic layers were washed with brine (2×15 mL), dried (Na₂SO₄). After filtration, the filtrate was concentrated in vacuo. The crude product was purified by reverse flash column chromatography with the following conditions: Column, C18; mobile phase, water (0.1% NH₄HCO₃) and MeCN (20% MeCN up to 30% in 9 min); Detector, UV 254/220 nm. This resulted in the sub-title compound (ADI-1) (100 mg, 0.34 mmol, 69%, 95% Purity) as a white solid. m/z 291.1 (M+H)⁺ (ES+).

Step 2: 4′-Chloro-N-[2-hydroxy-5-({[(1R,2S)-2-hydroxycyclopentyl]amino}methyl)-3-(trifluoromethyl)phenyl]-2′-(4-methyl-1,2,4-triazol-3-yl)-[1,1′-biphenyl]-3-carboxamide (ADI-2). To a stirred mixture of the product from step 1 above (ADI-1) (100 mg, 1 Eq, 0.34 mmol) and intermediate (ADB-7) (108 mg, 1 Eq, 0.34 mmol) in DMF (3 mL) were added DIPEA (134 mg, 3 Eq, 1.03 mmol) and HATU (125 mg, 1.5 Eq, 0.52 mmol) at rt. The resulting mixture was stirred for 3 h at rt. The resulting mixture was diluted with water. The resulting mixture was extracted with DCM (3×20 mL). The combined organic layers were washed with brine (2×20 mL), dried (Na₂SO₄). After filtration, the filtrate was concentrated in vacuo. The crude product was purified by reverse flash column chromatography with the following conditions: Column, C18; mobile phase, water (0.1% NH₄HCO₃) and MeCN (25% MeCN up to 40% in 10 min); Detector, UV 254/220 nm. This resulted in the sub-title compound (ADI-2) (62 mg, 0.11 mmol, 31%, 90% Purity) as a white solid. m/z 586.2/588.2 (M+H)⁺ (ES+).

Step 3: (1S,2R)-2-[({2-[4′-chloro-2′-(4-methyl-1,2,4-triazol-3-yl)-[1,1′-biphenyl]-3-yl]-7-(trifluoromethyl)-1,3-benzoxazol-5-yl}methyl)amino]cyclopentan-1-ol (ADI-3). To a stirred mixture of the product from step 2 above (ADI-2) (40 mg, 1 Eq, 68 μmol) in 1,4-dimethylbenzene (3 mL) was added ToSH (52 mg, 4 Eq, 0.27 mmol) at rt under nitrogen atmosphere. The resulting mixture was stirred for 2 h at 120° C. under nitrogen atmosphere. The mixture was allowed to cool down to rt. The resulting mixture was diluted with water. The resulting mixture was extracted with DCM (3×10 mL). The combined organic layers were washed with brine (2×10 mL), dried (Na₂SO₄). After filtration, the filtrate was concentrated in vacuo. The crude product was purified by Prep-HPLC with the following conditions: Column: XBridge Prep OBD C18 Column, 30*150 mm, 5 μm; Mobile Phase A: water (0.1% NH₄HCO₃), Mobile Phase B: MeCN; Flow rate: 60 mL/min; Gradient: 45% B to 55% B in 8 min, UV 254/220 nm; RT: 7.35. This resulted in the title compound (ADI-3) (4 mg, 7.0 μmol, 10%, 97% Purity) as a white solid. m/z 568.2/570.2 (M+H)⁺ (ES+). ¹H NMR (300 MHz, DMSO-d6) δ 8.41 (s, 1H), 8.18-8.06 (m, 2H), 7.92 (t, J=1.7 Hz, 1H), 7.85-7.71 (m, 4H), 7.59 (t, J=7.8 Hz, 1H), 7.39 (d, J=7.7 Hz, 1H), 4.40 (s, 1H), 4.00-3.82 (m, 3H), 3.18 (s, 3H), 2.79 (s, 1H), 1.75-1.52 (m, 4H), 1.41 (s, 2H).

### Example 295: 2-[4′-Chloro-2′-(4-methyl-1,2,4-triazol-3-yl)-[1,1′-biphenyl]-3-yl]-1,3-benzoxazole-5-carboxylate (ADJ-2)

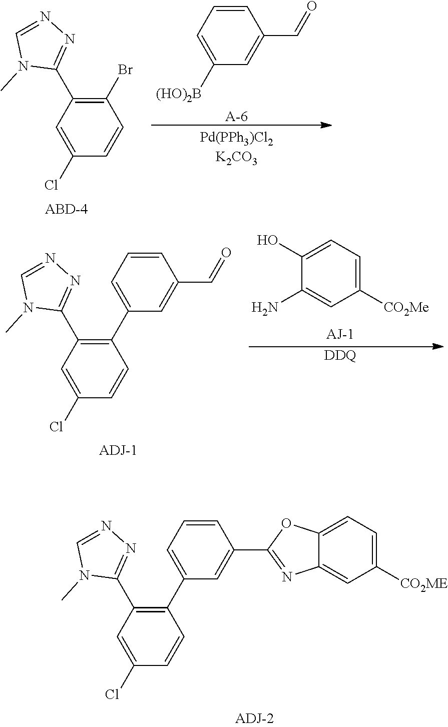

Step 1: 4′-Chloro-2′-(4-methyl-1,2,4-triazol-3-yl)-[1,1′-biphenyl]-3-carbaldehyde (ADJ-1). To a stirred mixture of intermediate (ABD-4) (540 mg, 1 Eq, 1.98 mmol) and 3-formylphenylboronic acid (A-6) (357 mg, 1.2 Eq, 2.38 mmol) in dioxane (20 mL) and H₂O (2 mL) were added K₂CO₃ (822 mg, 3 Eq, 5.94 mmol) at rt under nitrogen atmosphere. Then Pd(PPh₃)₂Cl₂ (139 mg, 0.1 Eq, 0.20 mmol) was added at rt under nitrogen atmosphere. The resulting mixture was stirred for 2 h at 100° C. under nitrogen atmosphere. The mixture was cooled to rt. The resulting mixture was diluted with water and extracted with DCM (3×80 mL). The combined organic layers were washed with brine (2×80 mL), dried (Na₂SO₄). After filtration, the filtrate was concentrated in vacuo. The resulting mixture was concentrated in vacuo. The crude product was purified by reverse flash column chromatography with the following conditions: Column, C18; mobile phase, water (0.1% NH₄HCO₃) and MeCN (20% MeCN up to 70% in 10 min); Detector, UV 254/220 nm to afford the sub-title compound (ADJ-1) (485 mg, 1.63 mmol, 82%, 90% Purity) as a white solid. m/z 298.1/300.1 (M+H)⁺ (ES+).

Step 2: Methyl 2-[4′-chloro-2′-(4-methyl-1,2,4-triazol-3-yl)-[1,1′-biphenyl]-3-yl]-1,3-benzoxazole-5-carboxylate (ADJ-2). A mixture of the product from step 1 above (ADJ-1) (30 mg, 1 Eq, 0.10 mmol) and methyl 3-amino-4-hydroxybenzoate (AJ-1) (20 mg, 1.2 Eq, 0.12 mmol) in DCM (5 mL) was stirred for 1 h at 60° C. under nitrogen atmosphere. To the above mixture was added DDQ (45 mg, 2 Eq, 0.20 mmol) at rt. The resulting mixture was stirred for additional 2 h at rt. The resulting mixture was diluted with water and extracted with EtOAc (3×10 mL). The combined organic layers were washed with brine (2×10 mL), dried (Na₂SO₄). After filtration, the filtrate was concentrated in vacuo. The crude product was purified by Prep-HPLC with the following conditions (Column: XBridge Prep OBD C18 Column, 30*150 mm, 5 μm; Mobile Phase A: water (0.1% NH₄HCO₃), Mobile Phase B: MeCN; Flow rate: 60 mL/min; Gradient: 42% B to 52% B in 9 min; Wave Length: 254/220 nm; RT: 8.67) to afford the title compound (ADJ-2) (2.7 mg, 6.1 μmol, 6.1%, 99% Purity) as a white solid. m/z 445.1/447.1 (M+H)⁺ (ES+). ¹H NMR 1H NMR (400 MHz, MeOH-d4) δ 8.38 (d, J=1.2 Hz, 2H), 8.27-8.20 (m, 1H), 8.17-8.04 (m, 2H), 7.86-7.65 (m, 4H), 7.58 (t, J=7.8 Hz, 1H), 7.47-7.40 (m, 1H), 3.96 (s, 3H), 3.17 (s, 3H).

### Example 296: {2-[4′-Chloro-2′-(4-methyl-1,2,4-triazol-3-yl)-[1,1′-biphenyl]-3-yl]-1,3-benzoxazol-5-yl}methanol (ADK-1)

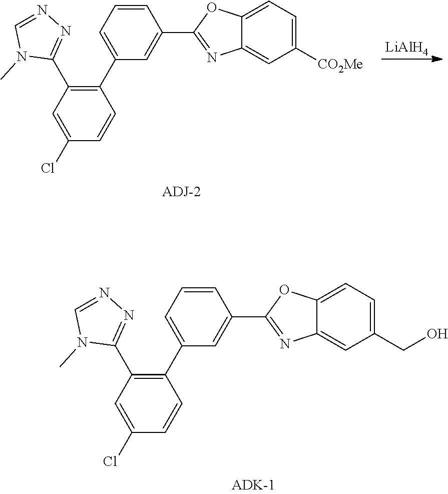 [SMILES: COC(=O)c1ccc2oc(-c3cccc(-c4ccc(Cl)cc4-c4nncn4C)c3)nc2c1 ; "LiAlH4" ; Cn1cnnc1-c1cc(Cl)ccc1-c1cccc(-c2nc3cc(CO)ccc3o2)c1]

To a stirred solution of compound (ADJ-2) (20 mg, 1 Eq, 45 μmol) in THF (2 mL) was added LiAlH₄ THF (5.12 uL, 1 M, 3 Eq, 0.14 mmol) at 0° C. under nitrogen atmosphere. The resulting mixture was stirred for 1 h at rt under nitrogen atmosphere. The reaction was then quenched by the addition of 1 mL of ice water at 0° C. The resulting mixture was concentrated in vacuo. The residue was purified by Prep-TLC with DCM/MeOH (20/1) and Prep-HPLC with the following conditions (Column: XBridge Prep OBD C18 Column, 30*150 mm, 5 μm; Mobile Phase A: water (0.1% NH₄HCO₃), Mobile Phase B: MeCN; Flow rate: 60 mL/min; Gradient: 50% B to 70% B in 8 min; Wave Length: 254/220 nm; RT: 6.62) to afford to afford the title compound (ADK-1) (4.4 mg, 11 μmol, 23%, 99% Purity) as a white solid. m/z 417.2/419.2 (M+H)⁺ (ES+). ¹H NMR (400 MHz, DMSO-d6) δ 8.43 (s, 1H), 8.16-8.12 (m, 1H), 8.00 (t, J=1.7 Hz, 1H), 7.84-7.79 (m, 1H), 7.79-7.71 (m, 4H), 7.54 (t, J=7.8 Hz, 1H), 7.42-7.36 (m, 1H), 7.30-7.24 (m, 1H), 5.34 (t, J=5.7 Hz, 1H), 4.63 (d, J=5.7 Hz, 2H), 3.12 (s, 3H).

### Example 297: (1S,2R)-2-[({2-[4′-Chloro-2′-(4-methyl-1,2,4-triazol-3-yl)-[1,1′-biphenyl]-3-yl]-1,3-benzoxazol-5-yl}methyl)amino]cyclopentan-1-ol (ADL-2)

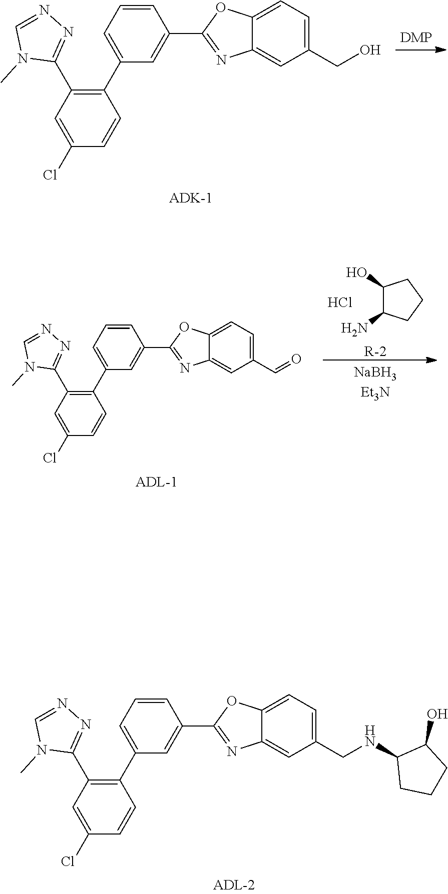

Step 1: 2-[4′-Chloro-2′-(4-methyl-1,2,4-triazol-3-yl)-[1,1′-biphenyl]-3-yl]-1,3-benzoxazole-5-carbaldehyde (ADL-1). To a stirred solution of compound (ADK-1) (40 mg, 1 Eq, 96 μmol) in DCM (5 mL) was added DMP (61 mg, 1.5 Eq, 0.14 mmol) at 0° C. under nitrogen atmosphere. The resulting mixture was stirred for 30 min at rt under nitrogen atmosphere. The resulting mixture was concentrated in vacuo. The residue was purified by Prep-TLC with DCM/MeOH (10/1) to afford the sub-title compound (ADL-1) (35 mg, 85 μmol, 79%, 95% Purity) as a white solid. m/z 415.1/417.1 (M+H)⁺ (ES+).

Step 2: (1S,2R)-2-[({2-[4′-Chloro-2′-(4-methyl-1,2,4-triazol-3-yl)-[1,1′-biphenyl]-3-yl]-1,3-benzoxazol-5-yl}methyl)amino]cyclopentan-1-ol (ADL-2). To a stirred solution of the product from step 1 above (ADL-1) (40 mg, 1 Eq, 96 μmol) and (1S,2R)-2-aminocyclopentan-1-ol, HCl (R-2) (15 mg, 1.1 Eq, 0.11 mmol) in DCM (5 mL) was added Et₃N (39 mg, 4 Eq, 0.38 mmol) at rt under nitrogen atmosphere. The resulting mixture was stirred for overnight at rt under nitrogen atmosphere. To the above mixture was added NaBH₄ (18 mg, 5 Eq, 0.48 mmol) at 0° C. The resulting mixture was stirred for additional 2 h at rt. The resulting mixture was concentrated in vacuo. The crude product was purified by Prep-HPLC with the following conditions (Column: XBridge Prep OBD C18 Column, 30*150 mm, 5 μm; Mobile Phase A: water (0.1% NH₄HCO₃), Mobile Phase B: MeCN; Flow rate: 60 mL/min; Gradient: 30% B to 45% B in 8 min; Wave Length: 254/220 nm; RT: 7.65) to afford the title compound (ADL-2) (10.1 mg, 20 μmol, 20%, 97% Purity) as a white solid. m/z 500.1/502.1 (M+H)⁺ (ES+). ¹H NMR (400 MHz, DMSO-d6) δ 8.42 (s, 1H), 8.17-8.11 (m, 1H), 7.98 (t, J=1.8 Hz, 1H), 7.82-7.69 (m, 5H), 7.55 (t, J=7.8 Hz, 1H), 7.46-7.40 (m, 1H), 7.32-7.26 (m, 1H), 4.44 (s, 1H), 3.99-3.92 (m, 1H), 3.93-3.76 (m, 2H), 3.12 (s, 3H), 2.82-2.73 (m, 1H), 1.73-1.53 (m, 4H), 1.47-1.34 (m, 2H).

### Example 298: (1S,2R)-2-{[(2-{6-Ethoxy-4-[4-fluoro-2-(4-methyl-1,2,4-triazol-3-yl)phenyl]pyridin-2-yl}-7-methoxy-1,3-benzoxazol-5-yl)methyl]amino}cyclopentan-1-ol (ADM-14)

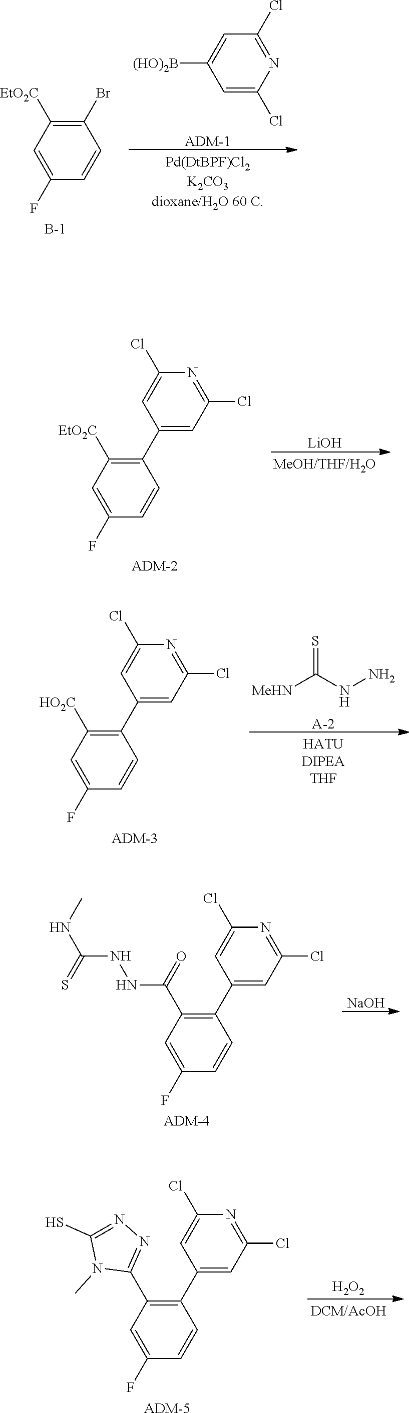

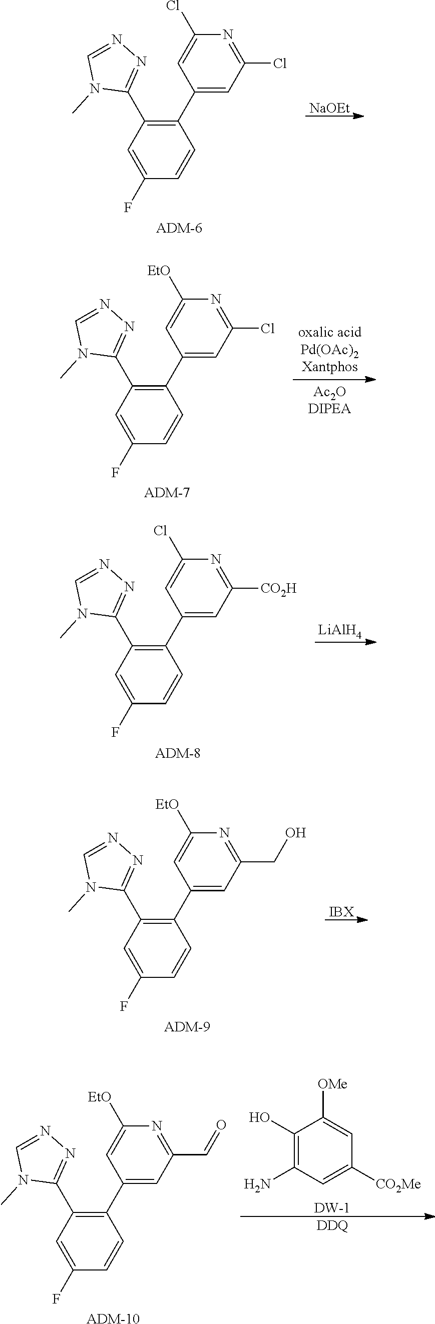

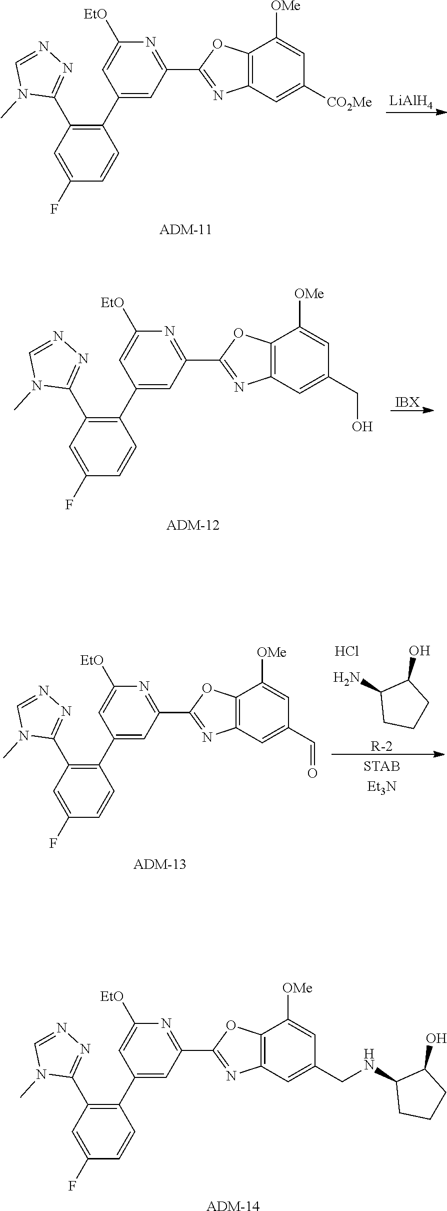

Step 1: Ethyl 2-(2,6-dichloropyridin-4-yl)-5-fluorobenzoate (ADM-2). To a stirred mixture of ethyl 2-bromo-5-fluorobenzoate (B-1) (4.94 g, 1 Eq, 20.0 mmol), (2,6-dichloropyridin-4-yl)boronic acid (ADM-1) (4.60 g, 1.2 Eq, 24.0 mmol) and K₂CO₃ (8.29 g, 3 Eq, 60.0 mmol) in 1,4-dioxane (80 mL) were added Pd(DtBPF)Cl₂ (1.30 g, 0.1 Eq, 2.00 mmol) at rt under nitrogen atmosphere. The resulting mixture was stirred for 5 h at 60° C. under nitrogen atmosphere. The resulting mixture was cooled to rt and diluted with water. The resulting mixture was extracted with EtOAc (3×300 mL). The combined organic layers were washed with brine (2×300 mL), dried (Na₂SO₄), filtered and concentrated in vacuo. The crude product was purified by reverse flash column chromatography with the following conditions: Column, C18; mobile phase, Water (0.1% NH₄HCO₃) and ACN (36% ACN up to 48% in 10 min); Detector, UV 254/220 nm. The product-containing fractions were combined and concentrated in vacuo. This resulted in the sub-title compound (ADM-2) (4.6 g, 14.7 mmol, 73%) as a brown solid. m/z 314.0/316.0 (M+H)⁺ (ES+).

Step 2: 2-(2,6-Dichloropyridin-4-yl)-5-fluorobenzoic acid (ADM-3). To a stirred mixture of the product from step 1 above (ADM-2) (2.3 g, 1 Eq, 7.32 mmol) in THF (30 mL) was added LiOH (700 mg, 4 Eq, 29.3 mmol) over 3 min at rt. The resulting mixture was stirred for additional 3 h at 60° C. The resulting mixture was cooled to rt. The residue was neutralized to pH 5 with HCl (aq., 1 M). The resulting mixture was diluted with water and extracted with EtOAc (3×200 mL). The combined organic layers were washed with brine (2×200 mL), dried (Na₂SO₄), filtered and concentrated in vacuo. The crude resulting mixture was used in the next step directly without further purification. m/z 286.0/288.0 (M+H)⁺ (ES+).

Step 3: 2-(2-(2,6-Dichloropyridin-4-yl)-5-fluorobenzoyl)-N-methylhydrazine-1-carbothioamide (ADM-4). To a stirred mixture of the product from step 2 above (ACM-3) (2.00 g, 1 Eq, 6.99 mmol) and 4-methyl-3-thiosemicarbazide (A-2) (740 mg, 1 Eq, 6.99 mmol) in THF (30 mL) were added DIPEA (2.71 g, 3 Eq, 21.0 mmol) and HATU (2.66 g, 1 Eq, 6.99 mmol) at rt. The resulting mixture was stirred for 2 h at rt. The crude resulting mixture was used in the next step directly without further purification. m/z 373.0/375.0 (M+H)⁺ (ES+).

Step 4: 5-(2-(2,6-Dichloropyridin-4-yl)-5-fluorophenyl)-4-methyl-4H-1,2,4-triazole-3-thiol (ADM-5). To a stirred mixture of the product from step 3 above (ADM-4) was added NaOH (aq., 10M) at rt. The resulting mixture was stirred for overnight at 100° C. The residue was cooled to rt and acidified to pH 5 with HCl (aq., 1M). The resulting mixture was diluted with water and extracted with EtOAc (3×100 mL). The combined organic layers were washed with brine (2×100 mL), dried (Na₂SO₄), filtered and concentrated in vacuo. The crude resulting mixture was used in the next step directly without further purification. m/z 355.0/357.0 (M+H)⁺ (ES+).

Step 5: 2,6-Dichloro-4-(4-fluoro-2-(4-methyl-4H-1,2,4-triazol-3-yl)phenyl)pyridine (ADM-6). To a stirred mixture of the product from step 4 above (ADM-5) (1 g, 1 Eq, 2.81 mmol) in DCM (10 mL) was added AcOH (340 mg, 2 Eq, 5.63 mmol) and hydrogen peroxide (480 g, 5 Eq, 14.1 mmol) at rt. The resulting mixture was stirred for 2 h at rt. The mixture was neutralized to pH 9 with saturated NaHCO₃ (aq.). The resulting mixture was diluted with water and extracted with EtOAc (3×100 mL). The combined organic layers were washed with brine (2×100 mL), dried (Na₂SO₄), filtered and concentrated in vacuo. The crude product was purified by reverse flash column chromatography with the following conditions: Column, C18; mobile phase, Water (0.1% NH₄HCO₃) and ACN (50% ACN up to 70% in 15 min); Detector, UV 254/220 nm. The product-containing fractions were combined and concentrated in vacuo. This resulted in the sub-title compound (ADM-6) (800 mg, 2.48 mmol, 88%) as a white solid. m/z 323.0/325.0 (M+H)⁺ (ES+).

Step 6: 2-Chloro-6-ethoxy-4-[4-fluoro-2-(4-methyl-1,2,4-triazol-3-yl)phenyl]pyridine (ADM-7). Into a 20 mL sealed tube were added the product from step 5 above (ADM-6) (300 mg, 1 eq, 0.93 mmol) in EtOH (10 mL) at rt. To the above mixture was added EtONa (94.76 mg, 1.5 eq, 1.39 mmol) over 1 min at rt. The resulting mixture was stirred for overnight at 60° C. The mixture was allowed to cool down to rt. The crude product was purified by reverse flash column chromatography with the following conditions: Column, C18; mobile phase, water (0.1% NH₄HCO₃) and MeCN (10% MeCN up to 70% in 15 min); Detector, UV 254/220 nm to afford the sub-title compound (ADM-7) (135 mg, 0.39 mmol, 44%, 95% Purity) as a yellow solid. m/z 333.1/335.1 (M+H)⁺ (ES+).

Step 7: 6-Ethoxy-4-[4-fluoro-2-(4-methyl-1,2,4-triazol-3-yl)phenyl]pyridine-2-carboxylic acid (ADM-8). Into a 100 mL round-bottom flask were added the product from step 6 above (ADM-7) (3.00 g, 1 Eq, 9.02 mmol), oxalic acid (1.22 g, 1.5 Eq, 13.5 mmol), Ac₂0 (1.38 g, 1.5 Eq, 13.5 mmol) and DIPEA (1.75 g, 1.5 Eq, 13.5 mmol) in DMF (30 mL) at rt under nitrogen atmosphere. To the above mixture was added Pd(OAc)₂ (200 mg, 0.1 Eq, 0.90 mmol) and XantPhos (1.04 g, 1.803 mmol, 0.2 Eq) at rt under nitrogen atmosphere. The resulting mixture was stirred for additional 2 h at 100° C. under nitrogen atmosphere. The mixture was allowed to cool down to rt. The crude product was purified by reverse flash column chromatography with the following conditions: Column, C18; mobile phase, Water (0.1% NH₄HCO₃) and MeCN (36% MeCN up to 48% in 10 min); Detector, UV 254/220 nm. The product-containing fractions were combined and concentrated in vacuo. This resulted in the sub-title compound (ADM-8) (950 mg, 2.79 mmol, 31%, 93% Purity) as a yellow solid. m/z 343.1 (M+H)⁺ (ES+).

Step 8: {6-Ethoxy-4-[4-fluoro-2-(4-methyl-1,2,4-triazol-3-yl)phenyl]pyridin-2-yl}methanol (ADM-9). Into a 40 mL sealed tube were added the product from step 7 above (ADM-9) (850 mg, 1 Eq, 2.48 mmol) in THF (9 mL) at rt. To the above mixture was added LiAlH₄ (283 mg, 3 Eq, 7.50 mmol) in portions over 2 min at 0° C. The resulting mixture was stirred for overnight at rt. The resulting mixture was filtered the filter cake was washed with EtOAc (3×15 mL). The filtrate was concentrated in vacuo. The crude product was purified by reverse flash column chromatography with the following conditions: Column, C18; mobile phase, water (0.1% NH₄HCO₃) and MeCN (20% MeCN up to 48% in 10 min); Detector, UV 254/220 nm. The product-containing fractions were combined and concentrated in vacuo. This resulted in the sub-title compound (ADM-9) (460 mg, 1.40 mmol, 56%, 90% Purity) as a yellow solid. m/z 329.1 (M+H)⁺ (ES+).

Step 9: 6-Ethoxy-4-[4-fluoro-2-(4-methyl-1,2,4-triazol-3-yl)phenyl]pyridine-2-carbaldehyde (ADM-10). Into a 20 mL sealed tube were added the product from step 8 above (ADM-9) (400 mg, 1 Eq, 1.22 mmol) in EtOAc (5 mL) at rt. To the above mixture was added IBX (1.02 g, 3 Eq, 3.65 mmol) over 2 min at rt. The resulting mixture was stirred for 2 h at rt. The resulting mixture was concentrated in vacuo to afford the sub-title compound (ADM-10) (320 mg, 0.98 mmol, 80%, 85% Purity) as a yellow solid. m/z 327.1 (M+H)⁺ (ES+).

Step 10: Methyl 2-{6-ethoxy-4-[4-fluoro-2-(4-methyl-1,2,4-triazol-3-yl)phenyl]pyridin-2-yl}-7-methoxy-1,3-benzoxazole-5-carboxylate (ADM-11). Into a 20 mL sealed tube were added the product from step 9 above (ADM-10) (250 mg, 1 Eq, 0.77 mmol) and methyl 3-amino-4-hydroxy-5-methoxybenzoate (DW-1) (151 mg, 1 Eq, 0.77 mmol) in DCM (3 mL) at rt. The resulting mixture was stirred for 1 h at rt. To the above mixture was added DDQ (174 mg, 1 Eq, 0.77 mmol) at rt. The resulting mixture was stirred for overnight at rt. The resulting mixture was concentrated in vacuo. The crude product was purified by reverse flash column chromatography with the following conditions: Column, C18; mobile phase, water (0.1% NH₄HCO₃) and MeCN (20% MeCN up to 58% in 12 min); Detector, UV 254/220 nm. The product-containing fractions were combined and concentrated in vacuo. This resulted in the sub-title compound (ADM-11) (190 mg, 0.38 mmol, 49%, 91% Purity) as a yellow solid. m/z 504.2 (M+H)⁺ (ES+).

Step 11: (2-{6-Ethoxy-4-[4-fluoro-2-(4-methyl-1,2,4-triazol-3-yl)phenyl]pyridin-2-yl}-7-methoxy-1,3-benzoxazol-5-yl)methanol (ADM-12). Into a 20 mL sealed tube were added the product from step 10 above (ADM-11) (150 mg, 1 Eq, 0.30 mmol) in THF (2 mL) at rt. To the above mixture was added LiAlH₄ (34 mg, 3 Eq, 0.89 mmol) in portions over 2 min at 0° C. The resulting mixture was stirred for overnight at rt. The reaction was then quenched by the addition of 3 mL of ice water at 0° C. The mixture was concentrated in vacuo. The crude product was purified by reverse flash column chromatography with the following conditions: Column, C18; mobile phase, water (0.1% NH₄HCO₃) and MeCN (20% MeCN up to 60% in 15 min); Detector, UV 254/220 nm. The product-containing fractions were combined and concentrated in vacuo. This resulted in the sub-title compound (ADM-12) (112 mg, 0.24 mmol, 79%, 93% Purity) as a yellow solid. m/z 476.2 (M+H)⁺ (ES+).

Step 12: 2-{6-Ethoxy-4-[4-fluoro-2-(4-methyl-1,2,4-triazol-3-yl)phenyl]pyridin-2-yl}-7-methoxy-1,3-benzoxazole-5-carbaldehyde (ADM-13). Into a 10 mL sealed tube were added the product from step 11 above (ADM-13) (100 mg, 1 Eq, 0.21 mmol) in EtOAc (2 mL) at rt. To the above mixture was added IBX (118 mg, 2 Eq, 0.42 mmol) in portions over 2 min at rt. The resulting mixture was stirred for additional 2 h at rt. The resulting mixture was concentrated in vacuo to afford the sub-title compound (ADM-13) (65 mg, 0.14 mmol, 65%, 87% Purity) as a yellow solid. m/z 474.2 (M+H)⁺ (ES+).

Step 13: (1S,2R)-2-{[(2-{6-Ethoxy-4-[4-fluoro-2-(4-methyl-1,2,4-triazol-3-yl)phenyl]pyridin-2-yl}-7-methoxy-1,3-benzoxazol-5-yl)methyl]amino}cyclopentan-1-ol (ADM-14). Into an 8 mL sealed tube were added the product from step 12 above (ADM-13) (30 mg, 1 Eq, 63 μmol), Et₃N (19 mg, 3 Eq, 0.19 mmol) and (1S,2R)-2-aminocyclopentan-1-ol, HCl (8 mg, 1.2 Eq, 76 μmol) in DCM (1 mL) at rt. The resulting mixture was stirred for 2 h at rt. To the above mixture was added NaBH(OAc)₃ (40 mg, 3 Eq, 0.19 mmol) in portions over 2 min at 0° C. The resulting mixture was stirred for 2 h at rt. The resulting mixture was concentrated in vacuo. The crude product was purified by Prep-HPLC with the following conditions: Column: SunFire Prep C18 OBD Column, 19×150 mm, 5 μm; Mobile Phase A: water (0.1% NH₄HCO₃+0.1% NH₃·H₂O), Mobile Phase B: MeCN; Flow rate: 60 mL/min; Gradient: 30 B to 60 B in 7 min; Detector, UV 254/210 nm; RT: 6.08 to afford the title compound (ADM-14) (3.0 mg, 5.4 μmol, 9.3%, 98% Purity) as a white solid. m/z 559.3 (M+H)⁺ (ES+) 1H NMR (400 MHz, Methanol-d4) δ 8.45 (s, 1H), 7.83-7.77 (m, 1H), 7.66 (d, J=1.4 Hz, 1H), 7.59-7.46 (m, 3H), 7.21 (d, J=1.4 Hz, 1H), 6.80 (d, J=1.3 Hz, 1H), 4.54-4.45 (m, 2H), 4.40-4.25 (m, 3H), 4.11 (s, 3H), 3.49-3.40 (m, 1H), 3.37 (s, 3H), 2.14-2.04 (m, 1H), 2.03-1.79 (m, 4H), 1.72-1.61 (m, 1H), 1.41 (t, J=7.1 Hz, 3H).

### Example 299: (2-Methoxyethyl)[(2-{3-[(1r,3s)-3-methyl-1-(4-methyl-1,2,4-triazol-3-yl)cyclobutyl]phenyl}-7-(trifluoromethyl)-1,3-benzoxazol-5-yl)methyl]amine (ADN-1)

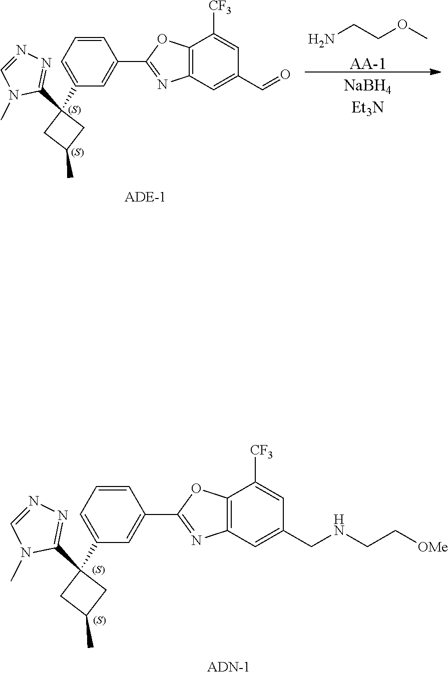

To a stirred solution of intermediate (ADE-1) (30 mg, 1 Eq, 68 μmol) and 2-methoxyethan-1-amine (AA-1) (15 mg, 3 Eq, 0.20 mmol) in MeOH (3 mL) was added Et₃N (21 mg, 3 Eq, 0.20 mmol) at rt. The resulting mixture was stirred for 2 h at 60° C. To the above mixture was added NaBH₄ (8 mg, 3 Eq, 0.20 mmol) at rt. The resulting mixture was stirred for 2 h at rt. The reaction was then quenched by the addition of 2 mL of MeOH at 0° C. The mixture was concentrated in vacuo. The residue was purified by Prep-TLC with DCM/MeOH (10/1). The crude product was purified by Prep-HPLC with the following conditions (Column: XBridge Prep OBD C18 Column, 30*150 mm, 5 μm; Mobile Phase A: water (0.1% FA), Mobile Phase B: MeCN; Flow rate: 60 mL/min; Gradient: 40% B to 50% B in 9 min; Wave Length: 254/220 nm; RT: 8.33) to afford the title compound (ADN-1) (6.8 mg, 14 μmol, 20%, 96% Purity) as a yellow oil. m/z 500.3 (M+H)⁺ (ES+). ¹H NMR (300 MHz, MeOH-d4) δ 8.32 (s, 1H), 8.26-8.24 (m, 1H), 8.21-8.17 (m, 1H), 8.11 (d, J=1.5 Hz, 1H), 7.84 (s, 1H), 7.74-7.67 (m, 2H), 4.29 (s, 2H), 3.65-3.61 (m, 2H), 3.40 (s, 3H), 3.29 (s, 3H), 3.15-3.11 (m, 2H), 3.02-2.98 (m, 2H), 2.69-2.64 (m, 3H), 1.20-1.16 (m, 3H).

### Example 300: 2-{6-Cyclopropyl-4-[4-fluoro-2-(4-methyl-1,2,4-triazol-3-yl)phenyl]pyridin-2-yl}-5-{[(3S)-3-methylpiperidin-1-yl]methyl}-7-(trifluoromethyl)-1,3-benzoxazole (ADO-1)

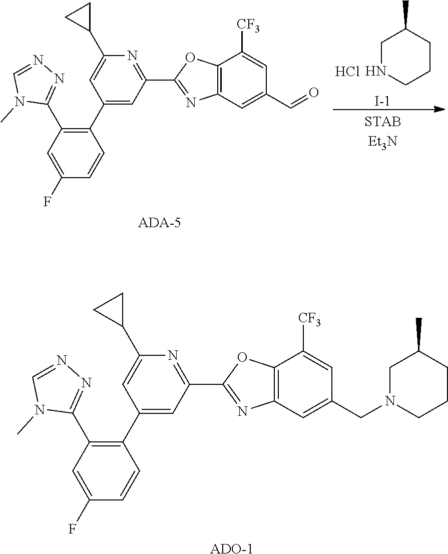

To a stirred mixture of intermediate (ADA-5) (20 mg, 39 μmol, 1 Eq) and (S)-3-methylpiperidine, HCl (I-1) (4 mg, 1 Eq, 39 μmol) in DCM (2 mL) was added DIPEA (15 mg, 3 Eq, 0.12 mmol) at rt. The resulting mixture was stirred for 1 h at 60° C. To the above mixture was added NaBH(OAc)₃ (42 mg, 5 Eq, 0.20 mmol) at rt. The resulting mixture was stirred for 1 h at rt under nitrogen atmosphere. The reaction was then quenched by the addition of 2 mL of MeOH at 0° C. The mixture was concentrated in vacuo. The crude product was purified by Prep-HPLC with the following conditions (Column: XBridge Prep OBD C18 Column, 30*150 mm, 5 μm; Mobile Phase A: water (0.1% NH₄HCO₃), Mobile Phase B: MeCN; Flow rate: 60 mL/min; Gradient: 60% B to 80% B in 8 min; Wave Length: 254/220 nm; RT: 7.83) to afford the title compound (ADO-1) (7.6 mg, 13 μmol, 33%, 97% Purity) as a white solid. m/z 591.3 (M+H)⁺ (ES+). ¹H NMR (400 MHz, MeOH-d4) δ 8.46 (s, 1H), 8.01 (s, 1H), 7.88-7.81 (m, 2H), 7.77 (s, 1H), 7.62-7.47 (m, 2H), 7.17-7.14 (m, 1H), 3.70 (s, 2H), 3.36 (s, 3H), 2.84 (t, J=12.1 Hz, 2H), 2.22-2.13 (m, 1H), 2.00 (t, J=11.3 Hz, 1H), 1.79-1.54 (m, 5H), 1.11-1.03 (m, 4H), 0.87 (d, J=5.7 Hz, 4H).

### Example 301: Methyl 2-{6-cyclopropyl-4-[4-fluoro-2-(4-methyl-1,2,4-triazol-3-yl)phenyl]pyridin-2-yl}-1,3-benzoxazole-5-carboxylate (ADP-1)

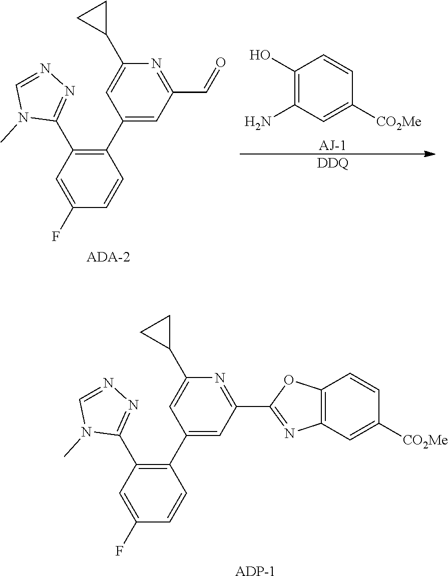

To a stirred solution of intermediate (ADA-2) (20 mg, 1 Eq, 62 μmol) in DCM (5 mL) was added methyl 3-amino-4-hydroxy-5-(trifluoromethyl)benzoate (AJ-1) (76 mg, 1.3 Eq, 0.32 mmol) at rt. The resulting mixture was stirred for 1 h at 60° C. The mixture was allowed to cool down to rt. To the above mixture was added DDQ (28 mg, 2 Eq, 0.12 mmol) at rt. The resulting mixture was stirred for 2 h at rt. The mixture was diluted with water and extracted with EtOAc (3×20 mL). The combined organic layers were washed with brine (2×20 mL), dried (Na₂SO₄). After filtration, the filtrate was concentrated in vacuo. The crude product was purified by Prep-HPLC with the following conditions (Column: XBridge Prep OBD C18 Column, 30*150 mm, 5 μm; Mobile Phase A: water (0.1% NH₄HCO₃), Mobile Phase B: MeCN; Flow rate: 60 mL/min; Gradient: 37% B to 60% B in 8 min; Wave Length: 254/220 nm; RT: 7.94) to afford the title compound (ADP-1) (2 mg, 4.3 μmol, 6.9%, 99% Purity) as a white solid. m/z 470.2 (M+H)⁺ (ES+). ¹H NMR (400 MHz, MeOH-d4) δ 8.43-8.41 (m, 2H), 8.19-8.16 (m, 1H), 7.92 (d, J=1.2 Hz, 1H), 7.86-7.80 (m, 2H), 7.61-7.49 (m, 2H), 7.13 (d, J=1.5 Hz, 1H), 3.96 (s, 3H), 3.30 (s, 3H), 2.20-2.10 (m, 1H), 1.08-1.05 (m, 4H).

### Example 302: (2-{6-Cyclopropyl-4-[4-fluoro-2-(4-methyl-1,2,4-triazol-3-yl)phenyl]pyridin-2-yl}-1,3-benzoxazol-5-yl)MeOH (ADQ-1)

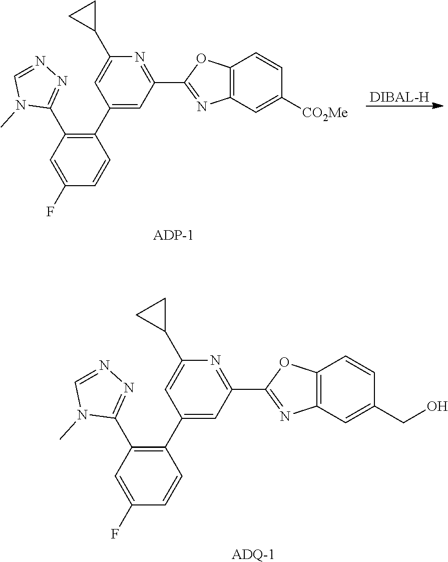

To a stirred mixture of compound (ADP-1) (26 mg, 1 Eq, 55 μmol) in THF (4 mL) was added DIBAL-H in THF (0.4 mL, 1.1 M, 8 Eq, 0.44 mmol) dropwise at 0° C. under nitrogen atmosphere. The resulting mixture was stirred for 1 h at 0° C. under nitrogen atmosphere. The reaction was then quenched by the addition of 2 mL of ice water at 0° C. The resulting mixture was diluted with water and extracted with DCM (3×20 mL). The combined organic layers were washed with brine (2×20 mL), dried (Na₂SO₄). After filtration, the filtrate was concentrated in vacuo. The crude product was purified by Prep-HPLC with the following conditions (Column: Sunfire prep C18 column, 30*150 mm, 5 μm; Mobile Phase A: water (0.1% FA), Mobile Phase B: MeCN; Flow rate: 60 mL/min; Gradient: 20% B to 50% B in 7 min; Wave Length: 254/220 nm; RT: 6.6) to afford the title compound (ADQ-1) (8.3 mg, 19 μmol, 34%, 99% Purity) as a white solid. m/z 442.0 (M+H)⁺ (ES+). ¹H NMR (300 MHz, DMSO-d6) δ 8.50 (s, 1H), 7.90-7.71 (m, 4H), 7.68-7.56 (m, 2H), 7.46-7.38 (m, 1H), 7.08 (d, J=1.6 Hz, 1H), 5.33 (t, J=5.8 Hz, 1H), 4.63 (d, J=5.6 Hz, 2H), 3.30 (s, 3H), 2.21-2.08 (m, 1H), 1.07-0.97 (m, 2H), 0.96-0.90 (m, 2H).

### Example 303: 1-((((2-(6-Cyclopropyl-4-(4-fluoro-2-(4-methyl-4H-1,2,4-triazol-3-yl)phenyl)pyridin-2-yl)-7-(trifluoromethyl)benzo[d]oxazol-5-yl)methyl)amino)methyl)cyclobutan-1-ol (ADR-1)

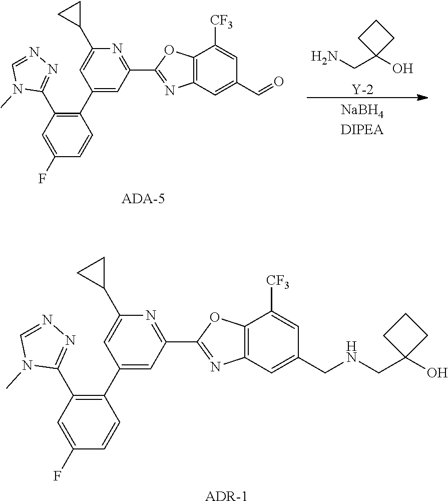

A mixture of intermediate (ADA-5) (30 mg, 1 Eq, 59 μmol) and 1-(aminomethyl)cyclobutan-1-ol (Y-2) (7 mg, 1.2 Eq, 71 μmol) in MeOH (5 mL) and DIPEA (23 mg, 3 Eq, 0.18 mmol) was stirred for overnight at rt. To the above mixture was added NaBH₄ (7 mg, 3 Eq, 0.18 mmol) at 0° C. The resulting mixture was stirred for 1 h at rt. The reaction was then quenched by the addition of 2 mL of MeOH at 0° C. The mixture was concentrated in vacuo. The crude product was purified by Prep-HPLC with the following conditions: Column: SunFire Prep C18 OBD Column, 19×150 mm, 5 μm; Mobile Phase A: water (0.1% NH₄HCO₃), Mobile Phase B: MeCN; Flow rate: 60 mL/min; Gradient: 20 B to 50 B in 7 min; Detector, UV 254/210 nm; RT: 6.08. This resulted in the title compound (ADR-1) (3.2 mg, 5.4 μmol, 9%, 99% Purity) as a white solid. m/z 593.2 (M+H)⁺ (ES+). ¹H NMR (400 MHz, MeOH-d4) δ 8.46 (s, 1H), 8.07 (s, 1H), 7.87-7.79 (m, 3H), 7.61-7.48 (m, 2H), 7.19-7.13 (m, 1H), 4.03 (s, 2H), 3.36 (s, 3H), 2.72 (s, 2H), 2.21-2.15 (m, 1H), 2.14-2.06 (m, 2H), 2.06-1.98 (m, 2H), 1.79-1.69 (m, 1H), 1.58-1.44 (m, 1H), 1.13-1.00 (m, 4H).

### Example 304: N-((2-(6-Cyclopropyl-4-(4-fluoro-2-(4-methyl-4H-1,2,4-triazol-3-yl)phenyl)pyridin-2-yl)-7-(trifluoromethyl)benzo[d]oxazol-5-yl)methyl)-2-methoxyethan-1-amine (ADS-1)

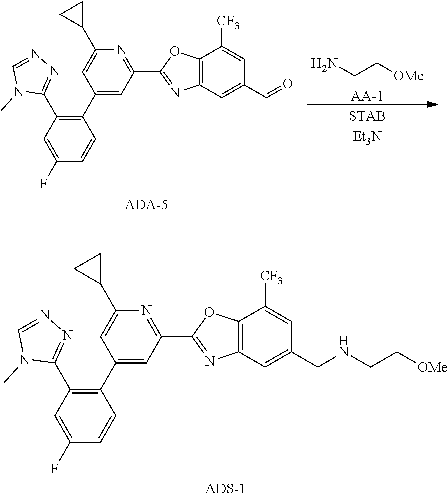

To a stirred mixture of intermediate (ADA-5) (20 mg, 1 Eq, 39 μmol) and 2-methoxyethan-1-amine (AA-1) (4 mg, 1.5 Eq, 58 μmol) in DCM (1 mL) was added Et₃N (12 mg, 3 Eq, 0.12 mmol) at rt. The resulting mixture was stirred for 1 h at 60° C. To the above mixture was added NaBH(OAc)₃ (42 mg, 5 Eq, 0.20 mmol) at rt. The resulting mixture was stirred for 2 h at rt under nitrogen atmosphere. The mixture was concentrated in vacuo. The crude product was purified by Prep-HPLC with the following conditions (Column: XBridge Prep OBD C18 Column, 30*150 mm, 5 μm; Mobile Phase A: water (0.1% NH₄HCO₃), Mobile Phase B: MeCN; Flow rate: 60 mL/min; Gradient: 38% B to 58% B in 8 min; Wave Length: 254/220 nm; RT: 7.77) to afford the title compound (ADS-1) (4.1 mg, 7.2 μmol, 18%, 97% Purity) as a yellow-green solid. m/z 567.3 (M+H)⁺ (ES+). ¹H NMR (400 MHz, MeOH-d4) δ 8.46 (s, 1H), 8.11-8.01 (m, 1H), 7.87-7.78 (m, 3H), 7.60-7.47 (m, 2H), 7.16 (d, J=1.6 Hz, 1H), 4.01 (s, 2H), 3.56-3.51 (m, 2H), 3.36 (s, 6H), 2.82 (t, J=5.3 Hz, 2H), 2.21-2.13 (m, 1H), 1.07 (d, J=6.5 Hz, 4H).

### Example 305: 1-({[(2-{4-[4-Chloro-2-(4-methyl-1,2,4-triazol-3-yl)phenyl]-6-cyclopropylpyridin-2-yl}-1,3-benzoxazol-5-yl)methyl]amino}methyl)cyclobutan-1-ol (ADT-12)

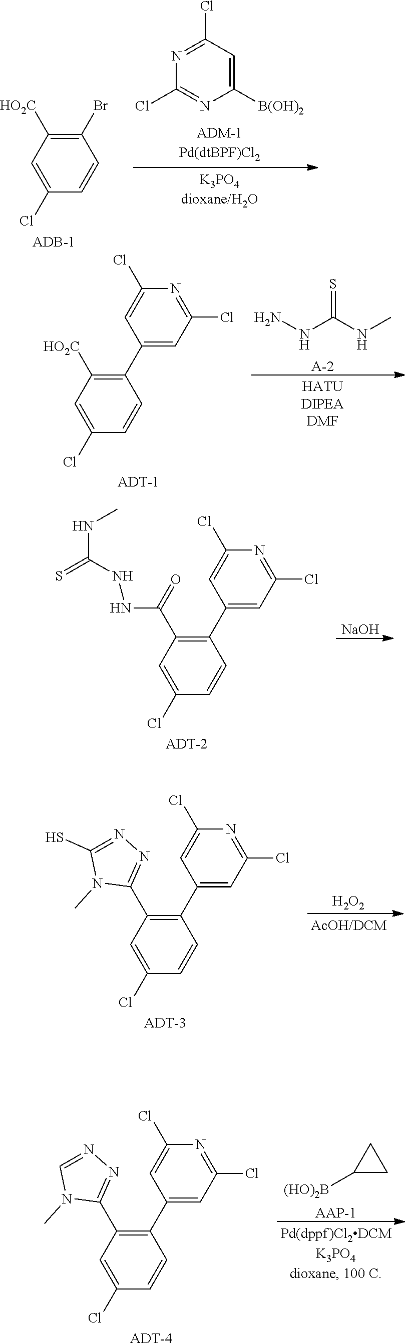

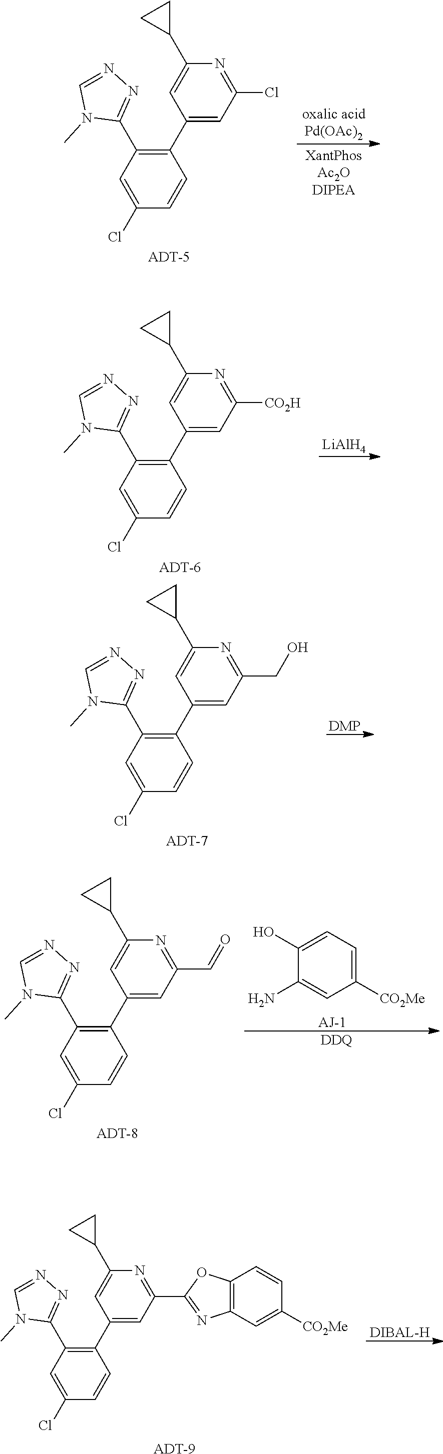

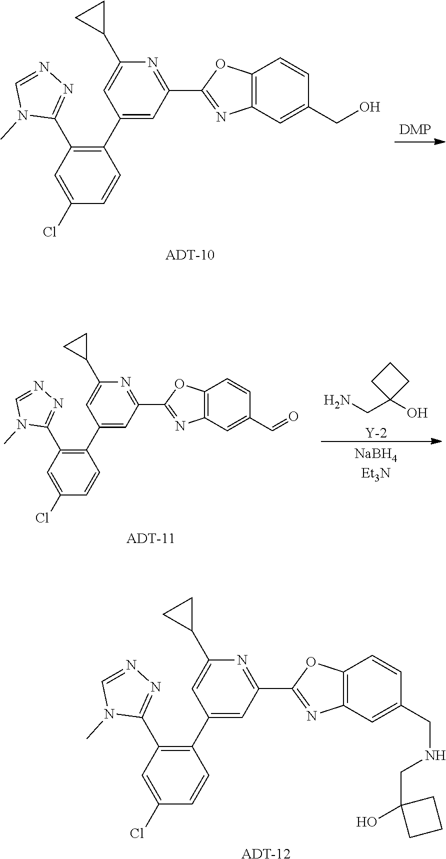

Step 1: 5-Chloro-2-(2,6-dichloropyridin-4-yl)benzoic acid (ADT-1). Into a 250-mL round-bottom flask purged and maintained with an inert atmosphere of nitrogen, was placed 2-bromo-5-chlorobenzoic acid (ADB-1) (5.00 g, 1 eq, 21.2 mmol), 2,6-dichloropyridin-4-ylboronic acid (ADM-1) (4.07 g, 1 eq, 21.2 mmol) and K₃PO₄ (13.5 g, 3 eq, 63.7 mmol) in 1,4-dioxane (50 mL) and water (10 mL), then Pd(DtBPF)Cl₂ (1.38 g, 0.1 eq, 2.12 mmol) was added at rt under nitrogen atmosphere. The resulting solution was stirred overnight at 60° C. under nitrogen atmosphere. The mixture was cooled to rt, diluted with water and extracted with EtOAc (3×300 mL). The combined organic layers were washed with brine (2×300 mL), dried (Na₂SO₄). After filtration, the filtrate was concentrated in vacuo. The crude product was purified by reverse flash column chromatography with the following conditions: Column, C18; mobile phase, water (0.1% NH₄HCO₃) and MeCN (10% MeCN up to 70% in 20 min); Detector, UV 254/220 nm. The product-containing fractions were combined and concentrated in vacuo. This resulted in of the sub-title compound (ADT-1) (1.8 g, 5.32 mmol, 28%, 89% Purity) as a brown solid. m/z 301.9/303.9 (M+H)⁺ (ES+).

Step 2: 2-(5-Chloro-2-(2,6-dichloropyridin-4-yl)benzoyl)-N-methylhydrazine-1-carbothioamide (ADT-2). Into a 100-mL round-bottom flask purged and maintained with an inert atmosphere of nitrogen, was placed the product from step 1 above (ADT-1) (1.80 g, 1 eq, 5.95 mmol) and DIPEA (2.31 g, 3 eq, 17.9 mmol) in DMF (40 mL) at rt, then 4-methyl-3-thiosemicarbazide (A-2) (630 mg, 1 eq, 5.95 mmol) and HATU (4.73 g, 2.5 eq, 14.9 mmol) were added at rt. The resulting solution was stirred for 6 h at rt. The resulting mixture was diluted with water and extracted with EtOAc (3×100 mL). The combined organic layers were washed with brine (2×100 mL), dried (Na₂SO₄). After filtration, the filtrate was concentrated in vacuo. The crude product was purified by reverse flash column chromatography with the following conditions: Column, C18; mobile phase, water (0.1% NH₄HCO₃) and MeCN (60% MeCN up to 85% in 10 min); Detector, UV 254/220 nm. The product-containing fractions were combined and concentrated in vacuo. This resulted in the sub-title compound (ADT-2) (720 mg, 1.70 mmol, 30%, 92% Purity) as a brown-yellow solid. m/z 389.0/391.0 (M+H)⁺ (ES+).

Step 3: 5-(5-Chloro-2-(2,6-dichloropyridin-4-yl)phenyl)-4-methyl-4H-1,2,4-triazole-3-thiol (ADT-3). Into a 50-mL round-bottom flask, was placed the product from step 2 above (ADT-2) (650 mg, 1 eq, 1.67 mmol) in aq. of NaOH (20 mL, 1 M). The resulting solution was stirred for 4 h at rt. The resulting solution was concentrated in vacuo. The crude product used directly in next step without any further purification. This resulted in the sub-title compound (ADT-3) (620 mg, 1.37 mmol, crude, 82% Purity) as brown-yellow solid. m/z 371.0/373.0 (M+H)⁺ (ES+).

Step 4: 2,6-Dichloro-4-(4-chloro-2-(4-methyl-4H-1,2,4-triazol-3-yl)phenyl)pyridine (ADT-4). Into a 50-mL round-bottom flask purged and maintained with an inert atmosphere of nitrogen, was placed the product from step 3 above (ADT-3) (600 mg, 1 eq, 1.61 mmol) in DCM (20 mL) at rt. Then AcOH (194 mg, 2 eq, 3.23 mmol) and H₂O₂ (912 mg, 30% Wt, 5 eq, 8.05 mmol) was added at rt. The resulting solution was stirred for 4 h at rt. The resulting mixture was diluted with water and extracted with DCM (3×80 mL). The combined organic layers were washed with brine (2×80 mL), dried (Na₂SO₄). After filtration, the filtrate was concentrated in vacuo. This resulted in the sub-title compound (ADT-4) (500 mg, 1.18 mmol, crude, 80%) as a brown-yellow solid. m/z 339.0/341.0 (M+H)⁺ (ES+).

Step 5: 2-Chloro-4-[4-chloro-2-(4-methyl-1,2,4-triazol-3-yl)phenyl]-6-cyclopropylpyridine (ADT-5). To a stirred solution of the product from step 4 above (ADT-4) (200 mg, 1 eq, 0.58 mmol), cyclopropylboronic acid (AAP-1) (75.8 mg, 1.5 eq, 0.88 mmol) and K₃PO₄ (250 mg, 2 eq, 1.17 mmol) in 1,4-dioxane (5 mL) was added Pd(dppf)Cl₂·DCM (43 mg, 0.05 mmol, 0.1 eq) at rt under nitrogen atmosphere. The resulting mixture was stirred for overnight at 100° C. under nitrogen atmosphere. The mixture was cooled to rt and diluted with water and extracted with EtOAc (3×50 mL). The combined organic layers were washed with brine (2×50 mL), dried (Na₂SO₄). After filtration, the filtrate was concentrated in vacuo. The crude product was purified by reverse flash column chromatography with the following conditions: Column, C18; mobile phase, water (0.1% FA) and MeCN (20% MeCN up to 70% in 10 min); Detector, UV 254/220 nm. The product-containing fractions were combined and concentrated in vacuo. This resulted in the sub-title compound (ADT-5) (110 mg, 0.29 mmol, 54%, 90% Purity) as a white solid. m/z 345.1/347.1 (M+H)⁺ (ES+).

Step 6: 4-[4-Chloro-2-(4-methyl-1,2,4-triazol-3-yl)phenyl]-6-cyclopropylpyridine-2-carboxylic acid (ADT-6). To a stirred solution of the product from step 5 above (ADT-5) (200 mg, 1 Eq, 0.57 mmol) and oxalic acid (78 mg, 1.5 Eq, 0.86 mmol) in 1,4-dioxane (10 mL) were added acetic anhydride (89 mg, 1.5 Eq, 0.86 mmol) and DIPEA (112 mg, 1.5 Eq, 0.86 mmol), XantPhos (67 mg, 0.2 Eq, 0.12 mmol) and Pd(OAc)₂ (13 mg, 0.1 Eq, 0.06 mmol) at rt under nitrogen atmosphere. The resulting mixture was stirred for 2 h at 100° C. under nitrogen atmosphere. The mixture was allowed to cool down to rt. The crude product was purified by reverse flash column chromatography with the following conditions: Column, C18; mobile phase, water (0.1% FA) and MeCN (10% MeCN up to 40% in 10 min); Detector, UV 254/220 nm. The product-containing fractions were combined and concentrated in vacuo. This resulted in the sub-title compound (ADT-6) (100 mg, 0.28 mmol, 49%, 90% Purity) as a yellow solid. m/z 355.1/357.1 (M+H)⁺ (ES+).

Step 7: {4-[4-Chloro-2-(4-methyl-1,2,4-triazol-3-yl)phenyl]-6-cyclopropylpyridin-2-yl}methanol (ADT-7). A solution of the product from step 6 above (ADT-6) (500 mg, 1 Eq, 1.40 mmol) in THF (15 mL) was added LiAlH₄ (160 mg, 3 Eq, 4.22 mmol) was stirred for 2 h at 0° C. under nitrogen atmosphere. The reaction was then quenched by the addition of 5 mL of ice water at 0° C. The resulting mixture was diluted with water and extracted with EtOAc (3×80 mL). The combined organic layers were washed with brine (2×80 mL), dried (Na₂SO₄). After filtration, the filtrate was concentrated in vacuo. The crude product was purified by reverse flash column chromatography with the following conditions: Column, C18; mobile phase, water (0.1% NH₄HCO₃) and MeCN (20% MeCN up to 60% in 10 min); Detector, UV 254/220 nm. The product-containing fractions were combined and concentrated in vacuo. This resulted in the sub-title compound (ADT-7) (250 mg, 52%, 93% Purity) as a yellow oil. m/z 341.1/343.1 (M+H)⁺ (ES+).

Step 8: 4-(4-Chloro-2-(4-methyl-4H-1,2,4-triazol-3-yl)phenyl)-6-cyclopropylpicolinaldehyde (ADT-8). A solution of the product from step 7 above (ADT-7) (500 mg, 1 Eq, 1.46 mmol) and Dess-Martin periodinane (809 mg, 1.3 Eq, 1.90 mmol) in THF (15 mL) was stirred for 2 h at rt under nitrogen atmosphere. The resulting mixture was filtered the filter cake was washed with EtOAc (3×5 mL). The filtrate was concentrated in vacuo. This resulted in the sub-title compound (ADT-8) (410 mg, 1.21 mmol, 89%, 90% Purity) as a white solid. m/z 339.1/341.1 (M+H)⁺ (ES+).

Step 9: Methyl 2-{4-[4-chloro-2-(4-methyl-1,2,4-triazol-3-yl)phenyl]-6-cyclopropylpyridin-2-yl}-1,3-benzoxazole-5-carboxylate (ADT-9). To a stirred solution of the product from step 8 above (ADT-8) (100 mg, 1 Eq, 0.29 mmol) and methyl 3-amino-4-hydroxybenzoate (AJ-1) (59 mg, 1.2 Eq, 0.35 mmol) in DCM (10 mL) The resulting mixture was stirred for 1 h at 60° C. To the above mixture was added DDQ (134 mg, 2 Eq, 0.59 mmol) at rt. The resulting mixture was stirred for 3 h at rt. The resulting mixture was diluted with water and extracted with EtOAc (3×50 mL). The combined organic layers were washed with brine (2×50 mL), dried (Na₂SO₄). After filtration, the filtrate was concentrated in vacuo. The residue was purified by Prep-TLC with EtOAc/petroleum ether (1/1). The product-containing fractions were combined and concentrated in vacuo. This resulted in the sub-title compound (ADT-9) (50 mg, 0.10 mmol, 35%, 90% Purity) as a white solid. m/z 486.1/488.1 (M+H)⁺ (ES+).

Step 10: (2-{4-[4-Chloro-2-(4-methyl-1,2,4-triazol-3-yl)phenyl]-6-cyclopropylpyridin-2-yl}-1,3-benzoxazol-5-yl)methanol (ADT-10). A solution of the product from step 9 above (ADT-9) (100 mg, 1 Eq, 0.20 mmol) in THF (10 mL) was added DIBAL-H in THF (2.0 mL, 1.0 M, 8 Eq, 1.60 mmol) at 0° C. The resulting mixture was stirred for 2 h at 0° C. under nitrogen atmosphere. The reaction was then quenched by the addition of 2 mL of ice water at 0° C. The resulting mixture was diluted with water and extracted with EtOAc (3×20 mL). The combined organic layers were washed with brine (2×20 mL), dried (Na₂SO₄). After filtration, the filtrate was concentrated in vacuo. The residue was purified by Prep-TLC with DCM/MeOH (10/1). This resulted in the sub-title compound (ADT-10) (70 mg, 0.15 mmol, 74%, 92% Purity) as a yellow oil. m/z 458.1/460.1 (M+H)⁺ (ES+).

Step 11: 2-{4-[4-Chloro-2-(4-methyl-1,2,4-triazol-3-yl)phenyl]-6-cyclopropylpyridin-2-yl}-1,3-benzoxazole-5-carbaldehyde (ADT-11). To a stirred solution of the product from step 10 above (ADT-10) (60 mg, 1 Eq, 0.13 mmol) and Dess-Martin periodinane (72 mg, 1.3 Eq, 0.17 mmol) in THF (4 mL) at rt. The resulting mixture was stirred for 2 h at rt. The resulting mixture was filtered the filter cake was washed with EtOAc (3×3 mL). The filtrate was concentrated in vacuo. This resulted in the sub-title compound (ADT-11) (40 mg, 88 μmol, 67%, 88% Purity) as a white solid. m/z 456.1/458.1 (M+H)⁺ (ES+).

Step 12: 1-({[(2-{4-[4-Chloro-2-(4-methyl-1,2,4-triazol-3-yl)phenyl]-6-cyclopropylpyridin-2-yl}-1,3-benzoxazol-5-yl)methyl]amino}methyl)cyclobutan-1-ol (ADT-12). To a stirred solution of the product from step 11 above (ADT-11) (50 mg, 1 Eq, 0.11 mmol) and 1-(aminomethyl)cyclobutan-1-ol (Y-2) (11. mg, 1 Eq, 0.11 mmol) in MeOH (5 mL) was added Et₃N (55 mg, 5 Eq, 0.55 mmol) at rt. The resulting mixture was stirred for 2 h at rt. To the above mixture was added NaBH₄ (41 mg, 10 Eq, 1.10 mmol) at 0° C. The resulting mixture was stirred for 2 h at rt. The reaction was then quenched by the addition of 2 mL of MeOH at 0° C. The resulting mixture was diluted with water and extracted with DCM (3×20 mL). The combined organic layers were washed with brine (2×20 mL), dried (Na₂SO₄). After filtration, the filtrate was concentrated in vacuo in vacuo. The crude product was purified by Prep-HPLC with the following conditions: Column: XBridge Prep OBD C18 Column, 30×150 mm, 5 μm; Mobile Phase A: water (0.1% NH₄HCO₃) Mobile Phase B: MeCN; Flow rate: 25 mL/min; Gradient: 30 B to 62 B in 9 min; Detector, UV 254/210 nm; RT: 8.15. The product-containing fractions were combined and evaporated partially in vacuo and lyophilized overnight to afford the title compound (ADT-12) (9.0 mg, 17 μmol, 15%, 97% Purity) as a white solid. m/z 541.3/543.3 (M+H)⁺ (ES+)¹H NMR (400 MHz, MeOH-d4) δ 8.45 (s, 1H), 7.90 (d, J=1.6 Hz, 1H), 7.88-7.71 (m, 4H), 7.69 (d, J=8.4 Hz, 1H), 7.50 (m, 1H), 7.10 (d, J=1.6 Hz, 1H), 3.98 (s, 2H), 3.33 (s, 3H), 2.74 (s, 2H), 2.20-2.10 (m, 1H), 2.10-1.99 (m, 2H), 1.79-1.68 (m, 2H), 1.54-1.51 (m, 1H), 1.49-1.48 (m, 1H), 1.06 (d, J=6.5 Hz, 4H).

### Example 306: (2-{6-Cyclopropyl-4-[4-fluoro-2-(4-methyl-1,2,4-triazol-3-yl)phenyl]pyridin-2-yl}-7-(trifluoromethyl)-1H-1,3-benzodiazol-5-yl)methanol (ADU-2)

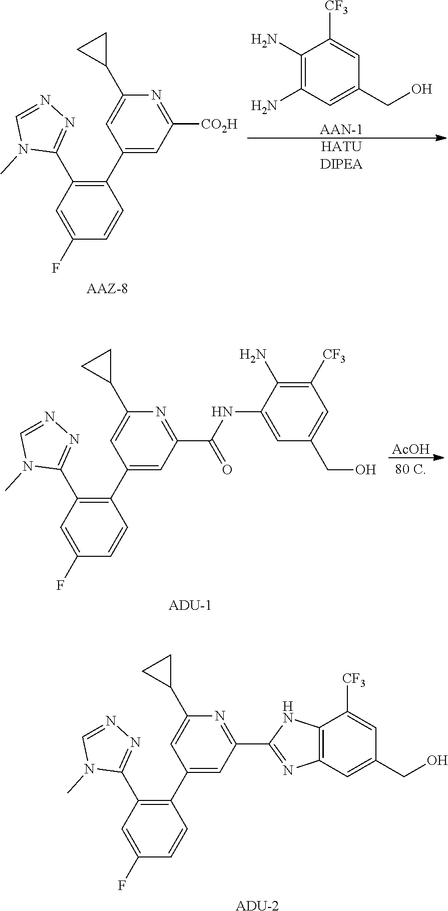

Step 1: N-[2-amino-5-(hydroxymethyl)-3-(trifluoromethyl)phenyl]-6-cyclopropyl-4-[4-fluoro-2-(4-methyl-1,2,4-triazol-3-yl)phenyl]pyridine-2-carboxamide (ADU-1). Into a 20 mL sealed tube were added intermediate (AAZ-8) (100 mg, 1 Eq, 0.30 mmol), DIPEA (115 mg, 3 Eq, 0.89 mmol) and intermediate (AAN-1) (91 mg, 1.5 Eq, 0.44 mmol) in THF (5 mL) at rt. To the above mixture was added HATU (225 mg, 2 Eq, 0.59 mmol) over 3 min at 0° C. The resulting mixture was stirred for 2 h at rt. The resulting mixture was concentrated in vacuo. The crude product was purified by reverse flash column chromatography with the following conditions: Column, C18; mobile phase, water (0.1% NH₄HCO₃) and MeCN (30% MeCN up to 60% in 15 min); Detector, UV 254/220 nm. The product-containing fractions were combined and concentrated in vacuo. This resulted in the sub-title compound (ADU-1) (101 mg, 0.19 mmol, 65%. 91% Purity) as a yellow solid. m/z 527.2 (M+H)⁺ (ES+).

Step 2: (2-{6-cyclopropyl-4-[4-fluoro-2-(4-methyl-1,2,4-triazol-3-yl)phenyl]pyridin-2-yl}-7-(trifluoromethyl)-1H-1,3-benzodiazol-5-yl)methanol (ADU-2). Into an 8 mL sealed tube were added the product from step 1 above (ADU-1) (50 mg, 1 Eq, 95 μmol) in AcOH (2 mL) at rt. The resulting mixture was stirred for 2 h at 80° C. The mixture was allowed to cool down to rt concentrated in vacuo. The crude product was purified by Prep-HPLC with the following conditions: Column: SunFire Prep C18 OBD Column, 19×150 mm, 5 μm; Mobile Phase A: water (0.1% NH₄HCO₃+0.1% NH₃·H₂O), Mobile Phase B: MeCN; Flow rate: 60 mL/min; Gradient: 30 B to 60 B in 7 min; Detector, UV 254/210 nm; RT: 6.08 to afford the title compound (ADU-2) (14.6 mg, 29 μmol, 30%, 99% Purity) as a white solid. m/z 509.1 (M+H)⁺ (ES+). ¹H NMR (400 MHz, MeOH-d4) δ 8.45 (s, 1H), 8.17 (s, 1H), 7.85 (d, J=6.4 Hz, 2H), 7.62-7.43 (m, 3H), 6.89 (d, J=1.6 Hz, 1H), 4.78 (s, 2H), 3.33 (s, 3H), 2.04 (d, J=10.6 Hz, 1H), 1.14-0.97 (m, 4H).

### Example 307: (1S,2R)-2-{[(2-{6-Cyclopropyl-4-[4-fluoro-2-(4-methyl-1,2,4-triazol-3-yl)phenyl]pyridin-2-yl}-7-(trifluoromethyl)-1H-1,3-benzodiazol-5-yl)methyl]amino}cyclopentan-1-ol (ADV-2)

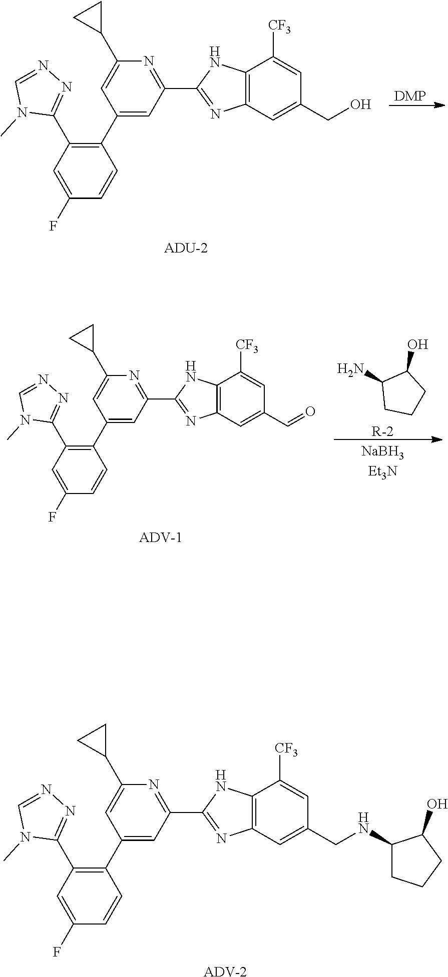

Step 1: 2-{6-Cyclopropyl-4-[4-fluoro-2-(4-methyl-1,2,4-triazol-3-yl)phenyl]pyridin-2-yl}-7-(trifluoromethyl)-1H-1,3-benzodiazole-5-carbaldehyde (ADV-1). To a stirred solution of compound (ADU-2) (60 mg, 1 Eq, 0.11 mmol) in THF (5 mL) was added Dess-Martin periodinane (65 mg, 1.3 Eq, 0.15 mmol) at rt. The resulting mixture was stirred for 1 h at rt. The resulting mixture was filtered the filter cake was washed with EtOAc (3×3 mL). The filtrate was concentrated in vacuo. This resulted in the sub-title compound (ADV-1) (50 mg, 0.10 mmol, 84%, 88% Purity) as a white solid. m/z 507.2 (M+H)⁺ (ES+).

Step 2: (1S,2R)-2-{[(2-{6-Cyclopropyl-4-[4-fluoro-2-(4-methyl-1,2,4-triazol-3-yl)phenyl]pyridin-2-yl}-7-(trifluoromethyl)-1H-1,3-benzodiazol-5-yl)methyl]amino}cyclopentan-1-ol (ADV-2). To a stirred solution of the product from step 1 above (ADV-1) (40 mg, 1 Eq, 0.07 mmol) and (1S,2R)-2-aminocyclopentan-1-ol (R-2) (16 mg, 2 Eq, 0.15 mmol) in DCM (5 mL) was added Et₃N (40 mg, 5 Eq, 0.40 mmol) at rt. The resulting mixture was stirred for overnight at rt. To the above mixture was added NaBH₄ (13 mg, 10 Eq, 1.58 mmol) at 0° C. The resulting mixture was stirred for 2 h at rt. The reaction was then quenched by the addition of 2 mL of MeOH at 0° C. The mixture was diluted with water and extracted with EtOAc (3×20 mL). The combined organic layers were washed with brine (2×20 mL), dried (Na₂SO₄). After filtration, the filtrate was concentrated in vacuo. The crude product was purified by Prep-HPLC with the following conditions: Column: XBridge Prep OBD C18 Column, 19×250 mm, 5 μm; Mobile Phase A: water (0.1% NH₄HCO₃) Mobile Phase B: MeCN; Flow rate: 25 mL/min; Gradient: 30 B to 65 B in 10 min; Detector, UV 254/210 nm; RT: 8.15. The product-containing fractions were combined and evaporated partially in vacuo and lyophilized overnight to afford the title compound (ADV-2) (5.8 mg, 9.8 μmol, 12.4%, 99% Purity) as a white solid. m/z 592.3 (M+H)⁺ (ES+). ¹H NMR (400 MHz, MeOH-d4) δ 8.45 (s, 1H), 8.16-7.81 (m, 3H), 7.80-7.68 (m, 1H), 7.61-7.44 (m, 2H), 6.96 (d, J=1.6 Hz, 1H), 4.40-4.21 (m, 3H), 3.38 (s, 4H), 2.14-2.01 (m, 2H), 2.02-1.72 (m, 4H), 1.72-1.60 (m, 1H), 1.16-0.96 (m, 4H).

### Example 308: [(2-{6-Cyclopropyl-4-[4-fluoro-2-(4-methyl-1,2,4-triazol-3-yl)phenyl]pyridin-2-yl}-7-(trifluoromethyl)-1H-1,3-benzodiazol-5-yl)methyl](2-methoxyethyl)amine (ADW-1)

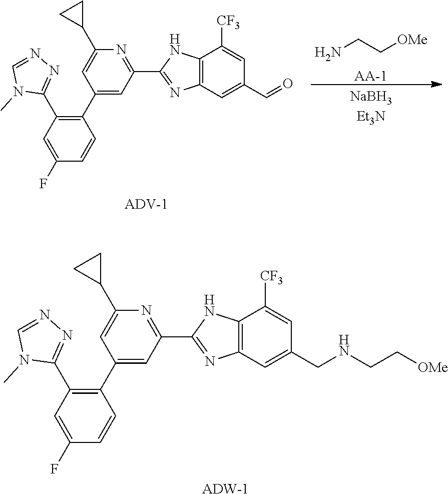

To a stirred solution of intermediate (ADV-1) (40 mg, 1 Eq, 0.07 mmol) and 2-methoxyethan-1-amine (AA-1) (12 mg, 2 Eq, 0.16 mmol) in DCM (5 mL) was added Et₃N (40 mg, 5 Eq, 0.40 mmol) at rt. The resulting mixture was stirred for overnight at rt. To the above mixture was added NaBH₄ (13 mg, 10 Eq, 1.58 mmol) at 0° C. The resulting mixture was stirred for 2 h at rt. The reaction was then quenched by the addition of 2 mL of MeOH at 0° C. The mixture was diluted with water and extracted with EtOAc (3×20 mL). The combined organic layers were washed with brine (2×20 mL), dried (Na₂SO₄). After filtration, the filtrate was concentrated in vacuo. The crude product was purified by Prep-HPLC with the following conditions: Column: XBridge Prep OBD C18 Column, 19×250 mm, 5 μm; Mobile Phase A: water (0.1% NH₄HCO₃) Mobile Phase B: MeCN; Flow rate: 25 mL/min; Gradient: 30 B to 65 B in 10 min; Detector, UV 254/210 nm; RT: 8.15. The product-containing fractions were combined and evaporated partially in vacuo and lyophilized overnight to afford the title compound (ADW-1) (1.7 mg, 3 μmol, 3.8%, 96% Purity) as a white solid. m/z 566.2 (M+H)⁺ (ES+). ¹H NMR (400 MHz, MeOH-d4) δ 8.45 (s, 1H), 8.15 (s, 1H), 7.89-7.78 (m, 2H), 7.65-7.54 (m, 2H), 7.51-7.47 (m, 1H), 6.90 (d, J=1.6 Hz, 1H), 3.97 (s, 2H), 3.54 (t, J=5.4 Hz, 3H), 3.35 (s, 3H), 3.33 (s, 2H), 2.80 (t, J=5.3 Hz, 2H), 2.07 (d, J=5.4 Hz, 1H), 1.12-1.01 (m, 4H).

### Example 309: (2-{6-Chloro-4-[4-fluoro-2-(4-methyl-1,2,4-triazol-3-yl)phenyl]pyridin-2-yl}-7-(trifluoromethyl)-1H-1,3-benzodiazol-5-yl)methanol (ADX-3)

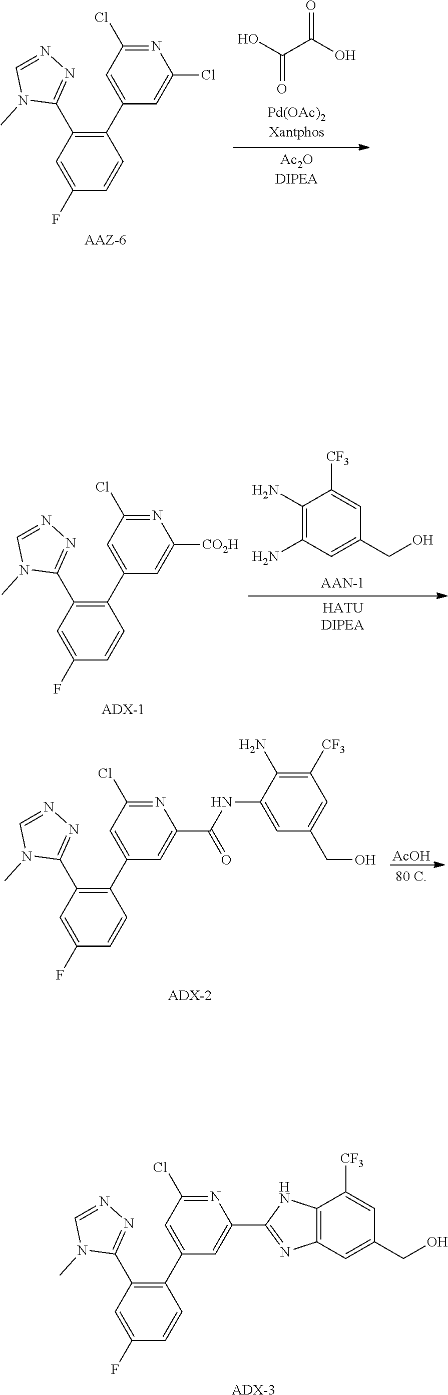

Step 1: 6-Chloro-4-[4-fluoro-2-(4-methyl-1,2,4-triazol-3-yl)phenyl]pyridine-2-carboxylic acid (ADX-1). To a stirred solution of intermediate (AAZ-6) (302 mg, 1 Eq, 0.93 mmol) and oxalic acid (126 mg, 1.5 Eq, 1.40 mmol) in DMF (10 mL) were added acetic anhydride (143 mg, 1.5 Eq, 1.40 mmol), DIPEA (181 mg, 1.5 Eq, 1.40 mmol), Pd(OAc)₂ (21 mg, 0.1 Eq, 0.09 mmol) and XantPhos (108 mg, 0.2 Eq, 0.19 mmol) at rt under nitrogen atmosphere. The resulting mixture was stirred for 2 h at 100° C. under nitrogen atmosphere. The mixture was cooled to rt and concentrated in vacuo. The crude product was purified by reverse flash column chromatography with the following conditions: Column, C18; mobile phase, water (0.1% FA) and MeCN (20% MeCN up to 60% in 10 min); Detector, UV 254/220 nm. The product-containing fractions were combined and concentrated in vacuo. This resulted in the sub-title compound (ADX-1) (200 mg, 0.60 mmol, 64%, 93% Purity) as a white solid. m/z 333.0/335.0 (M+H)⁺ (ES+).

Step 2: N-[2-Amino-5-(hydroxymethyl)-3-(trifluoromethyl)phenyl]-6-chloro-4-[4-fluoro-2-(4-methyl-1,2,4-triazol-3-yl)phenyl]pyridine-2-carboxamide (ADX-2). To a stirred solution of the product from step 1 above (ADX-1) (140 mg, 1 Eq, 0.42 mmol) and intermediate (AAN-1) (130 mg, 1.5 Eq, 0.63 mmol) in DMF (10 mL) was added DIPEA (272 mg, 5 Eq, 2.10 mmol) and HATU (175.9 mg, 1.1 Eq, 0.46 mmol) at rt. The resulting mixture was stirred for 2 h at rt. The crude product was purified by reverse flash column chromatography with the following conditions: Column, C18; mobile phase, water (0.1% NH₄HCO₃) and MeCN (20% MeCN up to 60% in 10 min); Detector, UV 254/220 nm. The product-containing fractions were combined and concentrated in vacuo. This resulted in the sub-title compound (ADX-2) (150 mg, 0.29 mmol, 68%, 92% Purity) as a white solid. m/z 521.1/523.1 (M+H)⁺ (ES+).

Step 3: (2-{6-Chloro-4-[4-fluoro-2-(4-methyl-1,2,4-triazol-3-yl)phenyl]pyridin-2-yl}-7-(trifluoromethyl)-1H-1,3-benzodiazol-5-yl)methanol (ADX-3). To a stirred solution of the product from step 2 above (ADX-2) (40 mg, 1 Eq, 0.07 mmol) in AcOH (3 mL) at rt under nitrogen atmosphere. The resulting mixture was stirred for overnight at 100° C. under nitrogen atmosphere. The mixture was cooled to rt and concentrated in vacuo. The crude product was purified by reverse flash column chromatography with the following conditions: Column, C18; mobile phase, water (0.1% NH₄HCO₃) and MeCN (20% MeCN up to 70% in 10 min); Detector, UV 254/220 nm. The product-containing fractions were combined and concentrated in vacuo. The crude product was purified by Prep-HPLC with the following conditions: Column: XBridge Prep OBD C18 Column, 19×250 mm, 5 μm; Mobile Phase A: water (0.1% NH₄HCO₃) Mobile Phase B: MeCN; Flow rate: 25 mL/min; Gradient: 30 B to 62 B in 9 min; Detector, UV 254/210 nm; RT: 8.15. The product-containing fractions were combined and evaporated partially in vacuo and lyophilized overnight to afford the title compound (ADX-3) (2.4 mg, 4.7 μmol, 6.2%, 97% Purity) as a white solid. m/z 503.1/505.1 (M+H)⁺ (ES+). 1H NMR (400 MHz, Methanol-d4) δ 8.49 (s, 1H), 8.27 (s, 1H), 7.88 (m, 1H), 7.83 (s, 1H), 7.64-7.51 (m, 3H), 7.23 (s, 1H), 4.78 (s, 2H), 3.50 (s, 3H).

### Example 310: (1S,2R)-2-{[(2-{6-Chloro-4-[4-fluoro-2-(4-methyl-1,2,4-triazol-3-yl)phenyl]pyridin-2-yl}-7-(trifluoromethyl)-1H-1,3-benzodiazol-5-yl)methyl]amino}cyclopentan-1-ol (ADY-2)

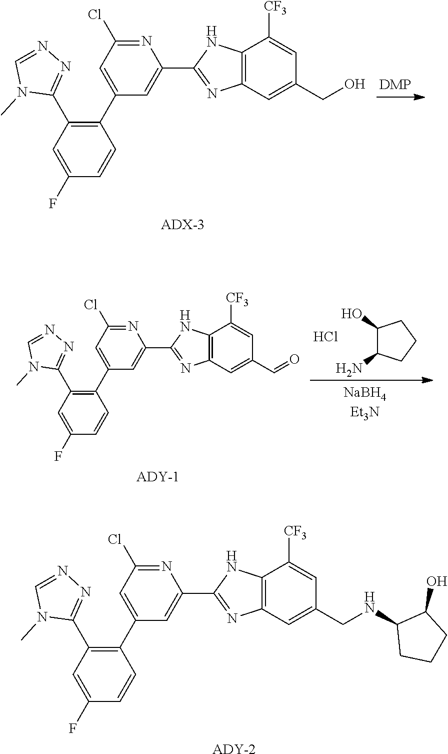

Step 1: 2-{6-Chloro-4-[4-fluoro-2-(4-methyl-1,2,4-triazol-3-yl)phenyl]pyridin-2-yl}-7-(trifluoromethyl)-1H-1,3-benzodiazole-5-carbaldehyde (ADY-1). A solution of compound (ADX-3) (50 mg, 1 Eq, 0.10 mmol) and Dess-Martin periodinane (55 mg, 1.3 Eq, 0.13 mmol) in THF (5 mL) was stirred for 1 h at rt. The resulting mixture was filtered the filter cake was washed with EtOAc (3×5 mL). The filtrate was concentrated in vacuo. This resulted in the sub-title compound (ADY-1) (40 mg, 0.08 mmol, 80%, 90% Purity) as a white solid. m/z 501.1/503.1 (M+H)⁺ (ES+).

Step 2: (1S,2R)-2-{[(2-{6-Chloro-4-[4-fluoro-2-(4-methyl-1,2,4-triazol-3-yl)phenyl]pyridin-2-yl}-7-(trifluoromethyl)-1H-1,3-benzodiazol-5-yl)methyl]amino}cyclopentan-1-ol (ADY-2). To a stirred solution of the product from step 1 above (ADY-1) (30 mg, 1 Eq, 0.06 mmol) and (1S,2R)-2-aminocyclopentan-1-ol, HCl (R-2) (12 mg, 2 Eq, 0.12 mmol) in DCM (5 mL) was added Et₃N (30 mg, 5 Eq, 0.30 mmol) at rt. The resulting mixture was stirred for overnight at rt. To the above mixture was added NaBH₄ (23 mg, 10 Eq, 0.60 mmol) at 0° C. The resulting mixture was stirred for 2 h at rt. The reaction was then quenched by the addition of 2 mL of MeOH at 0° C. The mixture was diluted with water and extracted with EtOAc (3×20 mL). The combined organic layers were washed with brine (2×20 mL), dried (Na₂SO₄). After filtration, the filtrate was concentrated in vacuo. The crude product was purified by Prep-HPLC with the following conditions: Column: XBridge Prep OBD C18 Column, 19×250 mm, 5 μm; Mobile Phase A: water (0.1% NH₄HCO₃) Mobile Phase B: MeCN; Flow rate: 25 mL/min; Gradient: 30 B to 62 B in 9 min; Detector, UV 254/210 nm; RT: 8.15. The product-containing fractions were combined and evaporated partially in vacuo and lyophilized overnight to afford the title compound (ADY-2) (5.9 mg, 10 μmol, 17%, 99% Purity) as a white solid. m/z 586.4/588.4 (M+H)⁺ (ES+). ¹H NMR (400 MHz, MeOH-d4) δ 8.22 (s, 1H), 7.96 (s, 1H), 8.10-7.81 (m, 2H), 7.75 (s, 1H), 7.64-7.52 (m, 2H), 7.30 (s, 1H), 4.37-4.29 (m, 3H), 3.53 (s, 3H), 3.25-1.63 (m, 7H).

### Example 311: (Cyclobutylmethyl)[(2-{3-[(1r,3s)-3-methyl-1-(4-methyl-1,2,4-triazol-3-yl)cyclobutyl]phenyl}-7-(trifluoromethyl)-1,3-benzoxazol-5-yl)methyl]amine (ADZ-1)

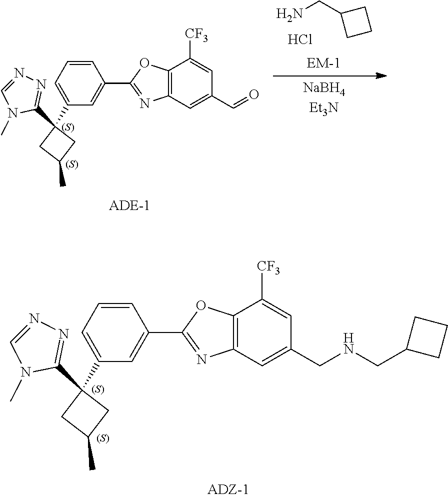

To a stirred solution of intermediate (ADE-1) (30 mg, 1 Eq, 68 μmol) and cyclobutylmethanamine, HCl (EM-1) (41 mg, 5 Eq, 0.34 mmol) in MeOH (3 mL) was added Et₃N (21 mg, 3 Eq, 0.20 mmol) at rt. The resulting mixture was stirred for 2 h at 60° C. To the above mixture was added NaBH₄ (8 mg, 3 Eq, 0.20 mmol) at rt. The resulting mixture was stirred for 2 h at rt. The reaction was then quenched by the addition of 2 mL of MeOH at 0° C. The mixture was concentrated in vacuo. The residue was purified by Prep-TLC with DCM/MeOH (10/1). The crude product was purified by Prep-HPLC with the following conditions (Column: XBridge Prep OBD C18 Column, 30*150 mm, 5 μm; Mobile Phase A: water (0.1% NH₄HCO₃+0.1% NH₃·H₂O), Mobile Phase B: MeCN; Flow rate: 60 mL/min; Gradient: 40% B to 50% B in 9 min; Wave Length: 254/220 nm; RT: 8.35) to afford the title compound (ADZ-1) (6.5 mg, 13 μmol, 19%, 99% Purity) as a yellow oil. m/z 510.2 (M+H)⁺ (ES+). ¹H NMR (300 MHz, MeOH-d4) δ 8.33-8.27 (m, 2H), 8.20-8.16 (m, 1H), 8.03 (s, 1H), 7.76 (s, 1H), 7.68-7.62 (m, 2H), 4.02 (s, 2H), 3.30 (s, 3H), 3.03-2.97 (m, 2H), 2.76-2.54 (m, 6H), 2.17-2.07 (m, 2H), 2.00-1.81 (m, 2H), 1.79-1.70 (m, 2H), 1.20-1.17 (m, 3H).

### Example 312: Methyl 7-methoxy-2-{3-[(1r,3s)-3-methyl-1-(4-methyl-1,2,4-triazol-3-yl)cyclobutyl]phenyl}-1,3-benzoxazole-5-carboxylate (AEA-1)

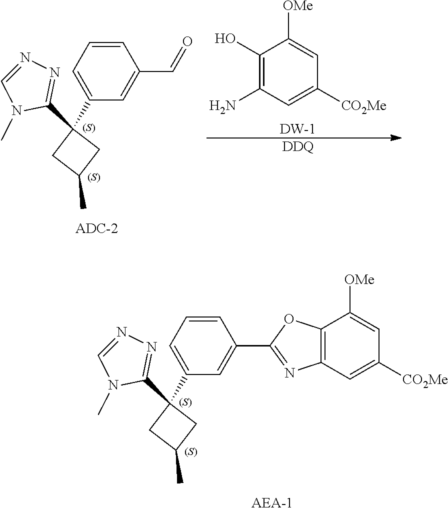

A solution of intermediate (ADC-2) (40 mg, 0.157 mmol, 1 Eq) and methyl 3-amino-4-hydroxy-5-methoxybenzoate (DW-1) (68 mg, 2.2 Eq, 0.35 mmol) in DCM (2 mL) was stirred for 1 h at 60° C. The mixture was allowed to cool down to rt. To the above mixture was added DDQ (53 mg, 1.5 Eq, 0.24 mmol) at rt. The resulting mixture was stirred for 2 h at rt. The mixture diluted with water and extracted with EtOAc (3×20 mL). The combined organic layers were washed with brine (2×20 mL), dried (Na₂SO₄). After filtration, the filtrate was concentrated in vacuo. The crude product was purified by Prep-HPLC with the following conditions (Column: Sunfire prep C18 column, 30*150 mm, 5 μm; Mobile Phase A: water (0.1% FA), Mobile Phase B: MeCN; Flow rate: 60 mL/min; Gradient: 35% B to 60% B in 7 min; Wave Length: 254/220 nm; RT: 6.52) to afford the title compound (AEA-1) (17.9 mg, 41 μmol, 26%, 99% Purity) as a yellow solid. m/z 433.3 (M+H)⁺ (ES+). ¹H NMR (300 MHz, MeOH-d4) δ 8.32-8.28 (m, 2H), 8.15 (d, J=3.3 Hz, 1H), 8.01-7.92 (m, 1H), 7.66-7.60 (m, 3H), 4.09 (s, 3H), 3.94 (s, 3H), 3.29 (s, 3H), 3.04-2.90 (m, 2H), 2.68-2.63 (m, 3H), 1.19-1.17 (m, 3H).

### Example 313: (7-Methoxy-2-{3-[(1r,3s)-3-methyl-1-(4-methyl-1,2,4-triazol-3-yl)cyclobutyl]phenyl}-1,3-benzoxazol-5-yl)methanol (AEB-1)

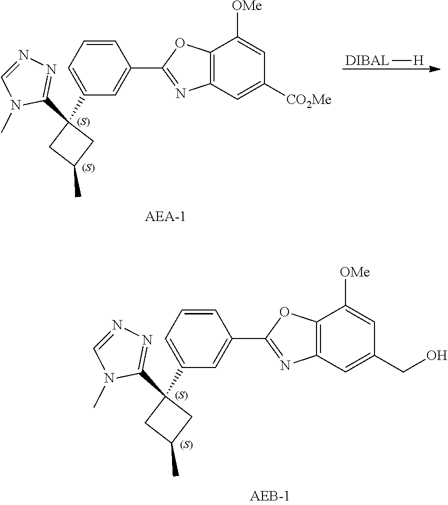

To a stirred solution of compound (AEA-1) (20 mg, 1 Eq, 43 μmol) in THF (2 mL) was added DIBAL-H in THF (0.19 mL, 1.1M, 5 Eq, 0.22 mmol) dropwise at 0° C. under nitrogen atmosphere. The resulting mixture was stirred for 1 h at 0° C. under nitrogen atmosphere. The reaction was then quenched by the addition of 1 mL of ice water at 0° C. The resulting mixture was diluted with water and extracted with DCM/MeOH (10/1) (3×10 mL). The combined organic layers were washed with brine (2×10 mL), dried (Na₂SO₄). After filtration, the filtrate was concentrated in vacuo. The crude product was purified by Prep-HPLC with the following conditions (Column: Sunfire prep C18 column, 30*150 mm, 5 μm; Mobile Phase A: water (0.1% FA), Mobile Phase B: MeCN; Flow rate: 60 mL/min; Gradient: 13% B to 29% B in 10 min; Wave Length: 254/220 nm; RT: 9.17) to afford the title compound (AEB-1) (6.9 mg, 17 μmol, 37%, 99 Purity) as a white solid. m/z 405.3 (M+H)⁺ (ES+). ¹H NMR (300 MHz, MeOH-d4) δ 8.32-8.27 (m, 2H), 8.16-8.13 (m, 1H), 7.65-7.55 (m, 2H), 7.32 (d, J=1.3 Hz, 1H), 7.04 (d, J=1.3 Hz, 1H), 4.71 (s, 2H), 4.05 (s, 3H), 3.30 (s, 3H), 3.04-2.95 (m, 2H), 2.75-2.60 (m, 3H), 1.20-1.15 (m, J=5.8 Hz, 3H).

### Example 314: (1S,2R)-2-{[(2-{3-[(1r,3s)-3-methyl-1-(4-methyl-1,2,4-triazol-3-yl)cyclobutyl]phenyl}-7-(trifluoromethyl)-1,3-benzoxazol-5-yl)methyl]amino}cyclopentan-1-ol (AEC-2)

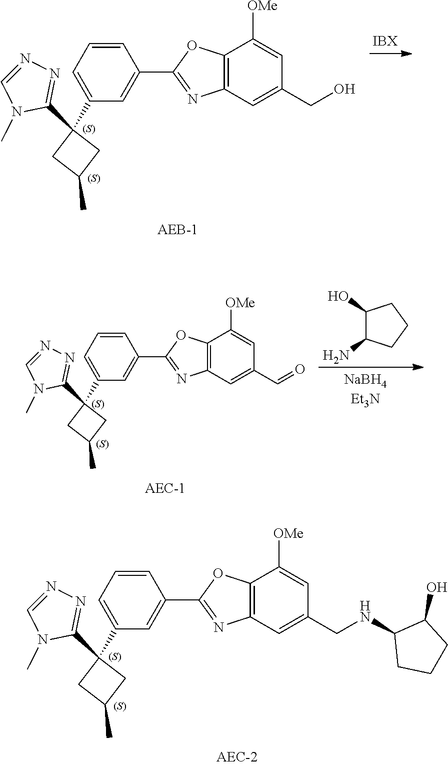

Step 1: 2-{3-[(1r,3s)-3-Methyl-1-(4-methyl-1,2,4-triazol-3-yl)cyclobutyl]phenyl}-7-(trifluoromethyl)-1,3-benzoxazole-5-carbaldehyde (AEC-1). To a stirred solution of compound (AEB-1) (180 mg, 1 Eq, 0.41 mmol) in EtOAc (18 mL) was added IBX (228 mg, 2 Eq, 0.81 mmol) at rt. The resulting mixture was stirred for 1 h at 70° C. The mixture was allowed to cool down to rt. The resulting mixture was filtered; the filter cake was washed with EtOAc (3×2 mL). The filtrate was concentrated in vacuo. This resulted in the sub-title compound (AEC-1) (150 mg, 0.37 mmol, 89%, 90% Purity) as a yellow solid. m/z 403.2 (M+H)⁺ (ES+).

Step 2: (1S,2R)-2-{[(2-{3-[(1r,3s)-3-Methyl-1-(4-methyl-1,2,4-triazol-3-yl)cyclobutyl]phenyl}-7-(trifluoromethyl)-1,3-benzoxazol-5-yl)methyl]amino}cyclopentan-1-ol (AEC-2). To a stirred solution of the product from step 1 above (AEC-1) (30 mg, 1 Eq, 68 μmol) and (1S,2R)-2-aminocyclopentan-1-ol, HCl (R-2) (28 mg, 3 Eq, 0.20 mmol) in MeOH (3 mL) was added Et₃N (21 mg, 3 Eq, 0.20 mmol) at rt. The resulting mixture was stirred for 2 h at 60° C. To the above mixture was added NaBH₄ (8 mg, 3 Eq, 0.20 mmol) at rt. The resulting mixture was stirred for 2 h at rt. The reaction was then quenched by the addition of 2 mL of MeOH at 0° C. The mixture was concentrated in vacuo. The residue was purified by Prep-TLC with DCM/MeOH (10/1). The crude product was purified by Prep-HPLC with the following conditions (Column: XBridge Prep OBD C18 Column, 30*150 mm, 5 μm; Mobile Phase A: water (0.1% NH₄HCO₃+0.1% NH₃·H₂O), Mobile Phase B: MeCN; Flow rate: 60 mL/min; Gradient: 40% B to 50% B in 9 min; Wave Length: 254/220 nm; RT: 8.35) to afford the title compound (AEC-2) (7.0 mg, 14 μmol, 20%, 97% Purity) as an off-white solid. m/z 488.4 (M+H)⁺ (ES+). ¹H NMR (300 MHz, MeOH-d4) δ 8.32 (s, 1H), 8.24 (d, J=1.8 Hz, 1H), 8.18-8.15 (m, 1H), 7.71-7.6 (m, 2H), 7.48 (d, J=1.4 Hz, 1H), 7.18 (d, J=1.4 Hz, 1H), 4.41-4.23 (m, 3H), 4.10 (s, 3H), 3.47-3.43 (m, 1H), 3.29 (s, 3H), 3.03-2.97 (m, 2H), 2.71-2.62 (m, 3H), 2.12-2.07 (m, 1H), 1.96-1.79 (m, 4H), 1.69-1.65 (m, 1H), 1.19-1.16 (m, 3H).

### Example 315: Methyl 2-(6-cyclopropyl-4-(4-fluoro-2-(4-methyl-4H-1,2,4-triazol-3-yl)phenyl)pyridin-2-yl)-1H-benzo[d]imidazole-5-carboxylate (AED-3)

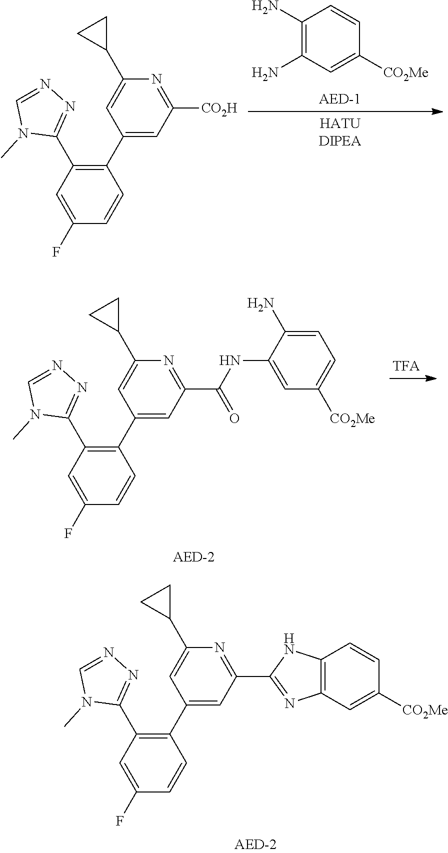 [SMILES: Cn1cnnc1-c1cc(F)ccc1-c1cc(C(=O)O)nc(C2CC2)c1 ; COC(=O)c1ccc(N)c(N)c1 ; COC(=O)c1ccc(N)c(NC(=O)c2cc(-c3ccc(F)cc3-c3nncn3C)cc(C3CC3)n2)c1 ; COC(=O)c1ccc2[nH]c(-c3cc(-c4ccc(F)cc4-c4nncn4C)cc(C4CC4)n3)nc2c1]

Step 1: Methyl 4-amino-3-(6-cyclopropyl-4-(4-fluoro-2-(4-methyl-4H-1,2,4-triazol-3-yl)phenyl)picolinamido)benzoate (AED-2). To a stirred solution of intermediate (AAZ-8) (246 mg, 1 Eq, 0.73 mmol) in DMF (8 mL) was added HATU (415 mg, 1.5 Eq, 1.09 mmol) and DIPEA (282 mg, 3 Eq, 2.18 mmol) at 0° C. The resulting mixture was stirred for 0.5 h at 0° C. To the above mixture was added methyl 3,4-diaminobenzoate (AED-1) (145 mg, 1.2 Eq, 0.87 mmol). The resulting mixture was stirred for 2 h at rt. The crude product was purified by reverse flash column chromatography with the following conditions: Column, C18; mobile phase, water (0.1% FA) and MeCN (10% MeCN up to 80% in 20 min); Detector, UV 254/220 nm to afford the sub-title compound (AED-2) (230 mg, 0.47 mmol, 65%, 92% Purity) as a brown oil. m/z 487.2 (M+H)⁺ (ES+).

Step 2: Methyl 2-(6-cyclopropyl-4-(4-fluoro-2-(4-methyl-4H-1,2,4-triazol-3-yl)phenyl)pyridin-2-yl)-1H-benzo[d]imidazole-5-carboxylate (AED-3). A solution of the product from step 1 above (AED-2) (20 mg, 1 Eq, 41 μmol) in TFA (1 mL) was stirred for overnight at 60° C. under nitrogen atmosphere. The mixture was allowed to cool down to rt and concentrated in vacuo. The crude product was purified by reverse flash column chromatography with the following conditions: Column, C18; mobile phase, water (0.1% NH₄HCO₃) and MeCN (10% MeCN up to 80% in 20 min); Detector, UV 254/220 nm and Prep-HPLC with the following conditions: (Column: XBridge Prep OBD C18 Column, 30*150 mm, 5 μm; Mobile Phase A: water (0.1% NH₄HCO₃), Mobile Phase B: MeCN; Flow rate: 60 mL/min; Gradient: 38% B to 48% B in 8 min; Wave Length: 254/220 nm; RT: 7.82). The product-containing fractions were combined and evaporated partially in vacuo and lyophilized overnight to afford the title compound (AED-3) (5.1 mg, 11 μmol, 26%, 98% Purity) as a white solid. m/z 469.2 (M+H)⁺ (ES+). ¹H NMR (400 MHz, MeOH-d4) δ 8.46 (s, 1H), 8.35 (s, 1H), 8.02-7.92 (m, 2H), 7.86-7.80 (m, 1H), 7.69 (s, 1H), 7.61-7.55 (m, 1H), 7.52-7.49 (m, 1H), 6.99 (d, J=1.6 Hz, 1H), 3.94 (s, 3H), 3.37 (s, 3H), 2.14-2.03 (m, 1H), 1.18-1.10 (m, 2H), 1.10-0.99 (m, 2H).

### Example 316: (2-(6-Cyclopropyl-4-(4-fluoro-2-(4-methyl-4H-1,2,4-triazol-3-yl)phenyl)pyridin-2-yl)-1H-benzo[d]imidazol-5-yl)methanol (AEE-1)

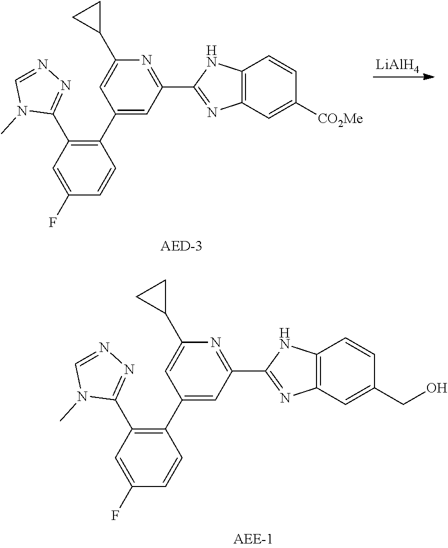 [SMILES: COC(=O)c1ccc2[nH]c(-c3cc(-c4ccc(F)cc4-c4nncn4C)cc(C4CC4)n3)nc2c1 ; "LiAlH4" ; Cn1cnnc1-c1cc(F)ccc1-c1cc(-c2nc3cc(CO)ccc3[nH]2)nc(C2CC2)c1]

To a stirred solution of compound (AED-3) (40 mg, 1 Eq, 85 μmol) in THF (4 mL) was added LiAlH₄ (19 mg, 6 Eq, 0.51 mmol) at 0° C. under nitrogen atmosphere. The resulting mixture was stirred for 2 h at rt under nitrogen atmosphere. The reaction was then quenched by the addition of 10 mL of ice water at 0° C. The resulting mixture was diluted with water and extracted with EtOAc (3×20 mL). The combined organic layers were washed with brine (2×20 mL), dried (Na₂SO₄). After filtration, the filtrate was concentrated in vacuo. The residue was purified by Prep-TLC with DCM/MeOH (10/1) and Prep-HPLC with the following conditions: (Column: XBridge Prep OBD C18 Column, 30*150 mm, 5 μm; Mobile Phase A: water (0.1% NH₄HCO₃), Mobile Phase B: MeCN; Flow rate: 60 mL/min; Gradient: 30% B to 40% B in 8 min; Wave Length: 254/220 nm; RT: 7.37). The product-containing fractions were combined and evaporated partially in vacuo and lyophilized overnight to afford the title compound (AEE-1) (5.7 mg, 13 μmol, 15%, 99% Purity) as a white solid. m/z 441.1 (M+H)⁺ (ES+). ¹H NMR (400 MHz, MeOH-d4) δ 8.45 (s, 1H), 7.93 (d, J=1.5 Hz, 1H), 7.86-7.78 (m, 1H), 7.70-7.54 (m, 3H), 7.53-7.47 (m, 1H), 7.30 (d, J=8.3 Hz, 1H), 6.91 (d, J=1.5 Hz, 1H), 4.73 (s, 2H), 3.35 (s, 3H), 2.12-2.01 (m, 1H), 1.16-1.09 (m, 2H), 1.09-0.98 (m, 2H).

### Example 317: (1S,2R)-2-(((2-(6-Cyclopropyl-4-(4-fluoro-2-(4-methyl-4H-1,2,4-triazol-3-yl)phenyl)pyridin-2-yl)-1H-benzo[d]imidazol-5-yl)methyl)amino)cyclopentan-1-ol (AEF-2)

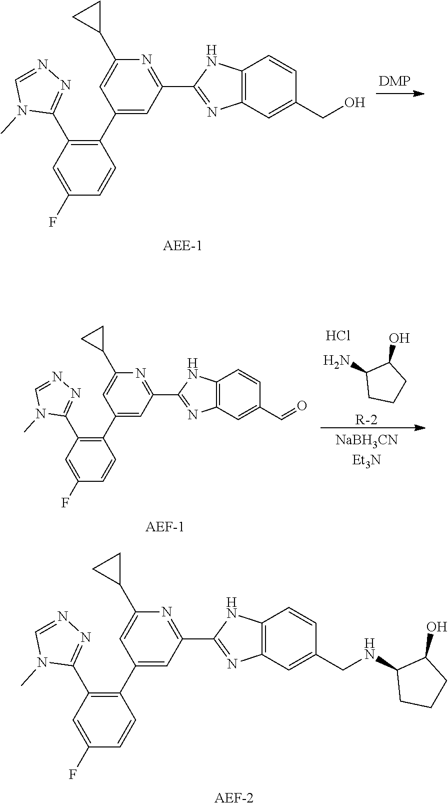

Step 1: 2-(6-Cyclopropyl-4-(4-fluoro-2-(4-methyl-4H-1,2,4-triazol-3-yl)phenyl)pyridin-2-yl)-1H-benzo[d]imidazole-5-carbaldehyde (AEF-1). To a stirred solution of compound (AEE-1) (66 mg, 1 Eq, 0.15 mmol) in THF (10 mL) was added Dess-Martin periodinane (76 mg, 1.2 Eq, 0.18 mmol) at 0° C. under nitrogen atmosphere. The resulting mixture was stirred for 2 h at rt under nitrogen atmosphere. The resulting mixture was concentrated in vacuo and purified by Prep-TLC with DCM/MeOH (15/1) to afford the sub-title compound (AEF-1) (50 mg, 0.12 mmol, 76%, 90% Purity) as a brown solid. m/z 439.2 (M+H)⁺ (ES+).

Step 2: (1S,2R)-2-(((2-(6-Cyclopropyl-4-(4-fluoro-2-(4-methyl-4H-1,2,4-triazol-3-yl)phenyl)pyridin-2-yl)-1H-benzo[d]imidazol-5-yl)methyl)amino)cyclopentan-1-ol (AEF-2). To a stirred mixture of the product from step 1 above (AEF-1) (30 mg, 1 Eq, 68 μmol) and (1S,2R)-2-aminocyclopentan-1-ol, HCl (R-2) (14 mg, 1.5 Eq, 0.10 mmol) in MeOH (4 mL) was added Et₃N (7 mg, 1 Eq, 68 μmol) at rt. The resulting mixture was stirred for 0.5 h at rt. To the above mixture was added AcOH (8 mg, 2 Eq, 0.14 mmol) and NaBH₃CN (9 mg, 2 Eq, 0.14 mmol) at rt. The resulting mixture was stirred for overnight at 60° C. The mixture was allowed to cool down to rt. The resulting mixture was concentrated in vacuo. The crude product was purified by reverse flash column chromatography with the following conditions: Column, C18; mobile phase, water (0.1% FA) and MeCN (10% MeCN up to 80% in 20 min); Detector, UV 254/220 nm and Prep-HPLC with the following conditions: (Column: XBridge Shield RP18 OBD Column, 30*150 mm, 5 μm; Mobile Phase A: water (0.1% FA), Mobile Phase B: MeCN; Flow rate: 60 mL/min; Gradient: 10% B to 37% B in 8 min; Wave Length: 254/220 nm; RT: 7.77). The product-containing fractions were combined and evaporated partially in vacuo and lyophilized overnight to afford the title compound (AEF-2) (9.3 mg, 18 μmol, 23%, 96% Purity) as a white solid. m/z 524.4 (M+H)⁺ (ES+). ¹H NMR (300 MHz, MeOH-d4) δ 8.45 (s, 1H), 7.92 (d, J=1.6 Hz, 1H), 7.86-7.76 (m, 2H), 7.71 (d, J=8.3 Hz, 1H), 7.63-7.48 (m, 2H), 7.42 (d, J=8.4 Hz, 1H), 6.99 (d, J=1.5 Hz, 1H), 4.43-4.25 (m, 3H), 3.37 (s, 4H), 2.13-1.77 (m, 6H), 1.73-1.60 (m, 1H), 1.19-0.98 (m, 4H).

### Example 318: (1S,2R)-2-{[(2-{4-[4-Fluoro-2-(4-methyl-1,2,4-triazol-3-yl)phenyl]pyridin-2-yl}-7-(trifluoromethyl)-1,3-benzoxazol-5-yl)methyl]amino}cyclopentan-1-ol (AEG-8)

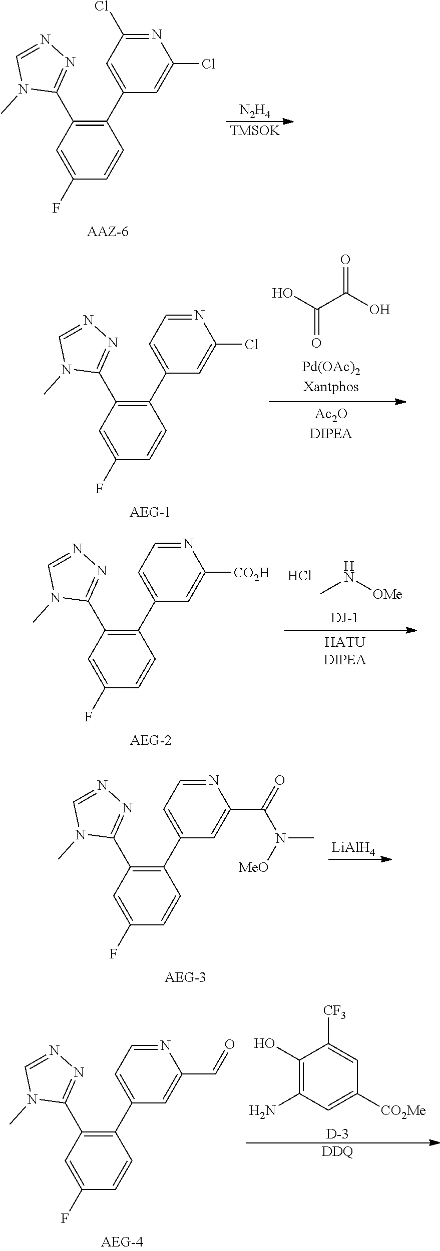

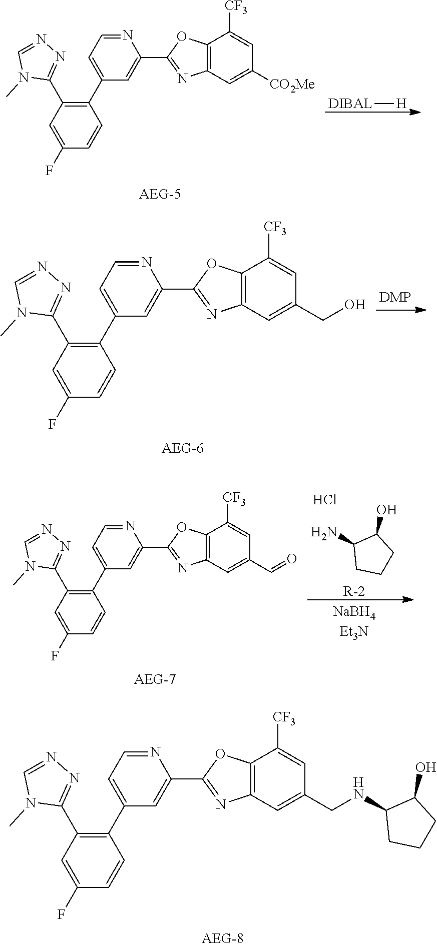

Step 1: 2-Chloro-4-[4-fluoro-2-(4-methyl-1,2,4-triazol-3-yl)phenyl]pyridine (AEG-1). To a stirred mixture of intermediate (AAZ-6) (1.00 g, 1 Eq, 3.10 mmol) in EtOH (20 mL) was added hydrazine hydrate (98% hydrazine) (4.65 g, 98% Wt, 30 Eq, 92.8 mmol) dropwise at rt. The resulting mixture was stirred for 3 h at 80° C. The mixture was allowed to cool down to rt and concentrated in vacuo. To the above mixture were added DMF (20 mL) and potassium trimethylsilanolate (592 mg, 1.5 Eq, 4.61 mmol) at 0° C. The resulting mixture was stirred for overnight at rt. The crude product was purified by reverse flash column chromatography with the following conditions: Column, C18; mobile phase, water (0.1% NH₄HCO₃) and MeCN (0% MeCN up to 40% in 30 min); Detector, UV 254/220 nm. This resulted in the sub-title compound (AEG-1) (750 mg, 2.60 mmol, 80%, 85% Purity) as a brown oil. m/z 289.1/291.1 (M+H)⁺ (ES+).

Step 2: 4-[4-Fluoro-2-(4-methyl-1,2,4-triazol-3-yl)phenyl]pyridine-2-carboxylic acid (AEG-2). To a solution of the product from step 1 above (AEG-1) (100 mg, 1 Eq, 0.35 mmol) and oxalic acid decahydrate (87 mg, 2 Eq, 0.69 mmol) in DMF (5 mL) were added Ac₂0 (53 mg, 1.5 Eq, 0.52 mmol) and DIPEA (67 mg, 1.5 Eq, 0.52 mmol), then Pd(OAc)₂ (16 mg, 0.2 Eq, 0.07 mmol) and XantPhos (80 mg, 0.4 Eq, 0.14 mmol) were added under nitrogen atmosphere. The resulting mixture was stirred for overnight at 100° C. under nitrogen atmosphere. The mixture was cooled to rt. The crude product was purified by reverse flash column chromatography with the following conditions: Column, C18; mobile phase, water (0.1% FA) and MeCN (0% MeCN up to 20% in 25 min); Detector, UV 254/220 nm. to afford the sub-title compound (AEG-2) (42 mg, 0.14 mmol, 37%, 90% Purity) as a brown oil. m/z 299.1 (M+H)⁺ (ES+).

Step 3: 4-[4-Fluoro-2-(4-methyl-1,2,4-triazol-3-yl) phenyl]-N-methoxy-N-methylpyridine-2-carboxamide (AEG-3). To a stirred solution of the product from step 2 above (AEG-2) (360 mg, 1 Eq, 1.21 mmol) and N, O-dimethylhydroxylamine, HCl (DJ-1) (141 mg, 1.2 Eq, 1.45 mmol) in THF (25 mL) were added DIPEA (468 mg, 3 Eq, 3.62 mmol) and HATU (597 mg, 1.3 Eq, 1.57 mmol) at rt under nitrogen atmosphere. The resulting mixture was stirred for 2 h at rt under nitrogen atmosphere. The residue was purified by Prep-TLC with DCM/MeOH (20/1) to afford the sub-title compound (AEG-3) (330 mg, 0.97 mmol, 72%, 91% Purity) as an off-white solid. m/z 342.1 (M+H)⁺ (ES+).

Step 4: 4-[4-Fluoro-2-(4-methyl-1,2,4-triazol-3-yl)phenyl]pyridine-2-carbaldehyde (AEG-4). To a stirred solution of the product from step 3 above (AEG-3) (180 mg, 1 Eq, 0.53 mmol) in THF (15 mL) was added LiAlH₄ (200 mg, 10 Eq, 5.27 mmol) at 0° C. under nitrogen atmosphere. The resulting mixture was stirred for 30 min at 0° C. under nitrogen atmosphere. The reaction was then quenched by the addition of 2 mL of ice water at 0° C. The mixture was concentrated in vacuo. The residue was purified by Prep-TLC with DCM/MeOH (20/1) to afford the sub-title compound (AEG-4) (83 mg, 0.22 mmol, 50%, 90% Purity) as an off-white solid. m/z 383.1 (M+H)⁺ (ES+).

Step 5: Methyl 2-{4-[4-fluoro-2-(4-methyl-1,2,4-triazol-3-yl) phenyl]pyridin-2-yl}-7-(trifluoromethyl)-1,3-benzoxazole-5-carboxylate (AEG-5). To a stirred solution of the product from step 4 above (AEG-4) (83 mg, 1 Eq, 0.29 mmol) and intermediate (D-3) (104 mg, 1.5 Eq, 0.44 mmol) in DCM (10 mL) at rt under nitrogen atmosphere. The resulting mixture was stirred for 1 h at 60° C. under nitrogen atmosphere. The mixture was allowed to cool down to rt. To the above mixture was added DDQ (133 mg, 2 Eq, 0.59 mmol) at 0° C. The resulting mixture was stirred for 1 h at rt. The resulting mixture was diluted with water and extracted with DCM (3×20 mL). The combined organic layers were washed with brine (2×20 mL), dried (Na₂SO₄). After filtration, the filtrate was concentrated in vacuo. The residue was purified by Prep-TLC with DCM/MeOH (15/1) to afford the sub-title compound (AEG-5) (75 mg, 0.15 mmol, 47%, 92% Purity) as a brown solid. m/z 498.1 (M+H)⁺ (ES+).

Step 6: (2-{4-[4-Fluoro-2-(4-methyl-1,2,4-triazol-3-yl)phenyl]pyridin-2-yl}-7-(trifluoromethyl)-1,3-benzoxazol-5-yl)methanol (AEG-6). To a stirred solution of the product from step 5 above (AEG-5) (20 mg, 1 Eq, 0.04 mmol) in THF (1 mL) was added DIBAL-H in THF (0.53 mL, 1.1 M, 10 Eq, 0.4 mmol) at −40° C. under nitrogen atmosphere. The resulting mixture was stirred for 30 min at -40° C. under nitrogen atmosphere. The reaction was then quenched by the addition of 0.5 mL of ice water at 0° C. The residue was purified by Prep-TLC with DCM/MeOH (10/1) to afford the sub-title compound (AEG-6) (8 mg, 17 μmol, 40%, 95% Purity) as an off-white solid. m/z 470.1 (M+H)⁺ (ES+).

Step 7: 2-{4-[4-Fluoro-2-(4-methyl-1,2,4-triazol-3-yl)phenyl]pyridin-2-yl}-7-(trifluoromethyl)-1,3-benzoxazole-5-carbaldehyde (AEG-7). To a stirred solution of the product from step 6 above (AEG-6) (32 mg, 1 Eq, 68 μmol) in DCM (5 mL) was added DMP (43 mg, 1.5 Eq, 0.10 mmol) at rt under nitrogen atmosphere. The resulting mixture was stirred for 30 min at rt under nitrogen atmosphere. The mixture was concentrated in vacuo. The residue was purified by Prep-TLC with DCM/MeOH (10/1) to afford the sub-title compound (AEG-7) (28 mg, 0.06 mmol, 83%, 92% Purity) as an off-white solid. m/z 468.1 (M+H)⁺ (ES+).

Step 8: (1S,2R)-2-{[(2-{4-[4-fluoro-2-(4-methyl-1,2,4-triazol-3-yl) phenyl]pyridin-2-yl}-7-(trifluoromethyl)-1,3-benzoxazol-5-yl)methyl]amino}cyclopentan-1-ol (AEG-8). To a stirred solution of the product from step 7 above (AEG-7) (28 mg, 1 Eq, 60 μmol) and (1S,2R)-2-aminocyclopentan-1-ol, HCl (R-2) (10 mg, 1.2 Eq, 72 μmol) in MeOH (5 mL) was added Et₃N (24 mg, 4 Eq, 0.24 mmol) at rt under nitrogen atmosphere. The resulting mixture was stirred for overnight at rt under nitrogen atmosphere. To the above mixture was added NaBH₄ (11 mg, 5 Eq, 0.30 mmol) at 0° C. The resulting mixture was stirred for 5 h at rt. The resulting mixture was concentrated in vacuo. The crude product was purified by Prep-HPLC with the following conditions (Column: XBridge Prep OBD C18 Column, 30*150 mm, 5 μm; Mobile Phase A: water (0.1% NH₄HCO₃+0.1% NH₃·H₂O), Mobile Phase B: MeCN; Flow rate: 60 mL/min; Gradient: 32% B to 42% B in 9 min; Wave Length: 254/220 nm; RT: 8.48) to afford the title compound (AEG-8) (2.1 mg, 3.8 μmol, 6.3%, 99% Purity) as a white solid. m/z 553.1 (M+H)⁺ (ES+). ¹H NMR (400 MHz, MeOH-d4) δ 8.73-8.70 (m, 1H), 8.47 (s, 1H), 8.13-8.04 (m, 2H), 7.89-7.81 (m, 2H), 7.64-7.52 (m, 2H), 7.43-7.40 (m, 1H), 4.17-4.13 (m, 1H), 4.03 (d, J=13.3 Hz, 1H), 3.96 (d, J=13.3 Hz, 1H), 3.42 (s, 3H), 2.99-2.91 (m, 1H), 1.94-1.68 (m, 4H), 1.61-1.46 (m, 2H).

### Example 319: (2-Methoxyethyl)[(2-{3-[(1r,3s)-3-methyl-1-(4-methyl-1,2,4-triazol-3-yl)cyclobutyl]phenyl}-7-(trifluoromethyl)-1,3-benzoxazol-5-yl)methyl]amine (AEH-2)

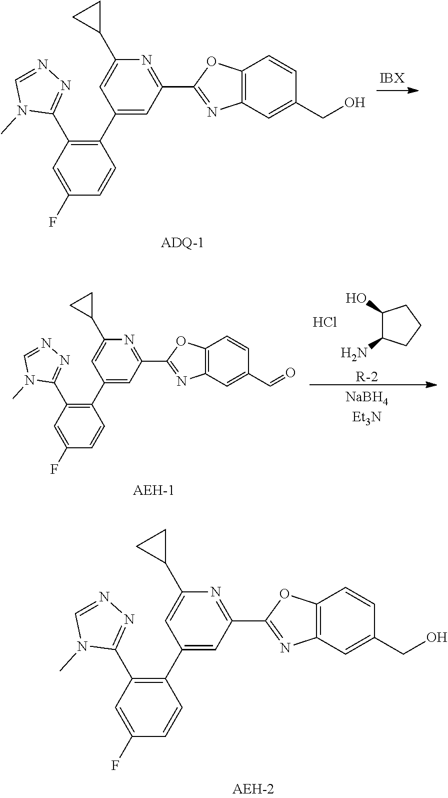

Step 1: 2-(6-Cyclopropyl-4-(4-fluoro-2-(4-methyl-4H-1,2,4-triazol-3-yl)phenyl)pyridin-2-yl)benzo[d]oxazole-5-carbaldehyde (AEH-1). To a stirred solution of compound (ADQ-1) (160 mg, 1 Eq, 0.41 mmol) in EtOAc (18 mL) was added IBX (228 mg, 2 Eq, 0.81 mmol) at rt. The resulting mixture was stirred for 1 h at 70° C. The mixture was allowed to cool down to rt. The resulting mixture was filtered the filter cake was washed with EtOAc (3×5 mL). The filtrate was concentrated in vacuo. This resulted in the sub-title compound (AEH-1) (130 mg, 0.30 mmol, 73%, 88% Purity) as a yellow solid. m/z 440.1 (M+H)⁺ (ES+).

Step 2: (1S,2R)-2-{[(2-{6-Cyclopropyl-4-[4-fluoro-2-(4-methyl-1,2,4-triazol-3-yl)phenyl]pyridin-2-yl}-1,3-benzoxazol-5-yl)methyl]amino}cyclopentan-1-ol (AEH-2). To a stirred solution of the product from step 1 above (AEH-1) (40 mg, 1 Eq, 79 μmol) and (1S,2R)-2-aminocyclopentan-1-ol, HCl (R-2) (28 mg, 3 Eq, 0.20 mmol) in MeOH (3 mL) were added Et₃N (24 mg, 3 Eq, 0.24 mmol) at rt. The resulting mixture was stirred for 2 h at 60° C. To the above mixture was added NaBH₄ (9 mg, 3 Eq, 0.24 mmol) at rt. The resulting mixture was stirred for overnight at rt. The reaction was then quenched by the addition of 2 mL of MeOH at 0° C. The residue was purified by Prep-TLC with DCM/MeOH (12/1). The crude product was purified by Prep-HPLC with the following conditions (Column: XBridge Prep OBD C18 Column, 30*150 mm, 5 μm; Mobile Phase A: water (0.1% NH₄HCO₃), Mobile Phase B: MeCN; Flow rate: 60 mL/min; Gradient: 30% B to 70% B in 9 min; Wave Length: 254/220 nm; RT: 8.18) to afford the title compound (AEH-2) (10.2 mg, 19 μmol, 28%, 99% Purity) as a white solid. m/z 525.3 (M+H)⁺ (ES+). ¹H NMR (400 MHz, MeOH-d4) δ 8.45 (s, 1H), 7.89 (d, J=1.6 Hz, 1H), 7.85-7.81 (m, 2H), 7.70-7.67 (m, 1H), 7.60-7.48 (m, 3H), 7.09 (s, 1H), 4.17 (s, 1H), 4.02-3.89 (m, 2H), 3.30 (s, 3H), 2.98 (s, 1H), 2.18-2.13 (m, 1H), 1.93-1.72 (m, 4H), 1.59-1.54 (m, 2H), 1.07-1.04 (m, 4H).

### Example 320: [(2-{6-Cyclopropyl-4-[4-fluoro-2-(4-methyl-1,2,4-triazol-3-yl)phenyl]pyridin-2-yl}-1,3-benzoxazol-5-yl)methyl](cyclopropylmethyl)amine (AEI-1)

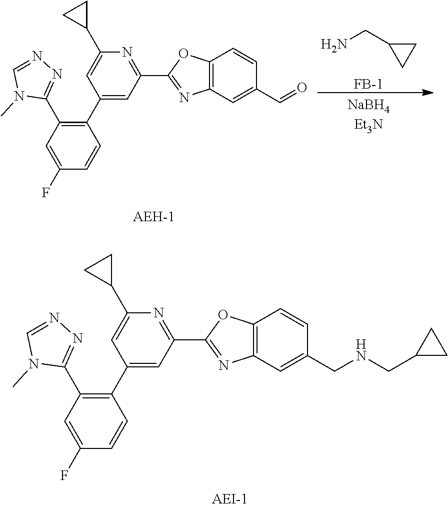

To a stirred mixture of intermediate (AEH-1) (30 mg, 1 Eq, 68 μmol) and 1-cyclopropylmethanamine (FB-1) (7 mg, 1.5 Eq, 0.10 mmol) in MeOH (5 mL) were added Et₃N (21 mg, 3 Eq, 0.20 mmol) at rt under nitrogen atmosphere. The resulting mixture was stirred for 1 h at 60° C. under nitrogen atmosphere. To the above mixture was added NaBH₄ (13 mg, 5 Eq, 0.34 mmol) at rt. The resulting mixture was stirred for 1 h at rt. The reaction was then quenched by the addition of 2 mL of MeOH at 0° C. The mixture was concentrated in vacuo. The residue was purified by Prep-TLC with DCM/MeOH (10/1). The crude product was purified by Prep-HPLC with the following conditions (Column: Xselect CSH C18 OBD Column 30*150 mm, 5 μm; Mobile Phase A: water (0.1% FA), Mobile Phase B: MeCN; Flow rate: 60 mL/min; Gradient: 8% B to 32% B in 7 min; Wave Length: 254/220 nm; RT: 6.5) to afford the title compound (AEI-1) (19.4 mg, 39 μmol, 57%, 97% Purity) as a white solid. m/z 495.2 (M+H)⁺ (ES+). ¹H NMR (300 MHz, DMSO-d6) δ 8.39 (s, 1H), 7.97 (d, J=1.6 Hz, 1H), 7.92-7.76 (m, 3H), 7.72-7.51 (m, 3H), 7.10 (d, J=1.5 Hz, 1H), 4.27 (s, 2H), 4.09 (s, 3H), 2.86 (d, J=7.3 Hz, 2H), 2.25-2.11 (m, 1H), 1.15-1.02 (m, 3H), 0.96-0.88 (m, 2H), 0.66-0.57 (m, 2H), 0.39-0.29 (m, 2H).

### Example 321: N-Ethyl-4-[4-fluoro-2-(4-methyl-1,2,4-triazol-3-yl)phenyl]-6-[7-(trifluoromethyl)-1,3-benzoxazol-2-yl]pyridin-2-amine (AEJ-4)

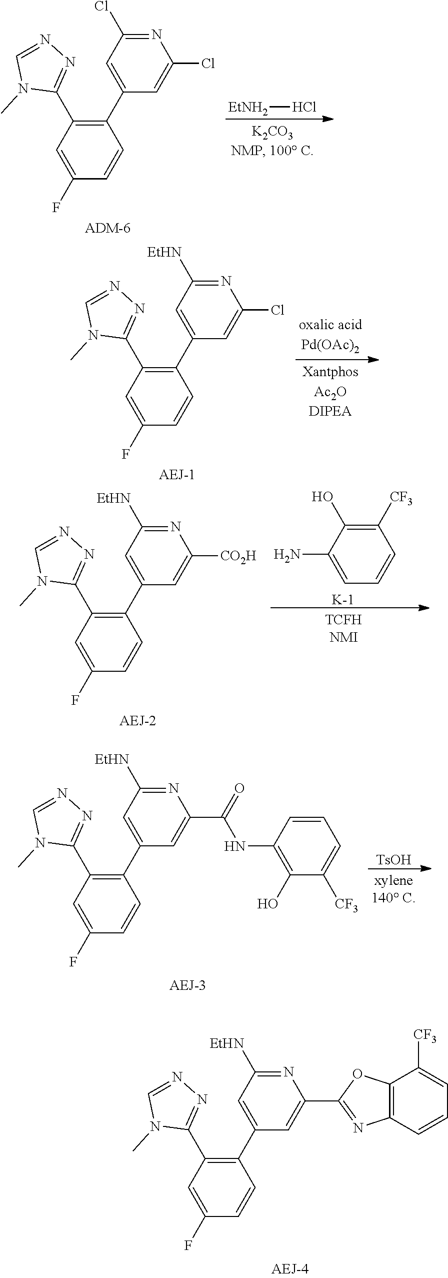

Step 1: 6-Chloro-N-ethyl-4-(4-fluoro-2-(4-methyl-4H-1,2,4-triazol-3-yl) phenyl) pyridin-2-amine (AEJ-1). To a stirred mixture of intermediate (ADM-6) (400 mg, 1 Eq, 1.24 mmol) and ethanamine, HCl (558 mg, 10 Eq, 12.4 mmol) in NMP (5 mL) were added K₂CO₃ (1.71 g, 10 Eq, 12.3 mmol) at rt. The resulting mixture was stirred for overnight at 100° C. The mixture was purified by reverse flash column chromatography with the following conditions: Column, C18; mobile phase, water (0.1% NH₄HCO₃) and MeCN (36% ACN up to 48% in 10 min); Detector, UV 254/220 nm. The product-containing fractions were combined and concentrated in vacuo. This resulted in the sub-title compound (AEJ-1) (300 mg, 906 μmol, 73%) as a white solid. m/z 332.1/334.1 (M+H)⁺ (ES+).

Step 2: 6-(Ethylamino)-4-[4-fluoro-2-(4-methyl-1,2,4-triazol-3-yl)phenyl]pyridine-2-carboxylic acid (AEJ-2). To a stirred solution of the product from step 1 above (AEJ-1) (250 mg, 1 Eq, 0.75 mmol) and oxalic acid (204 mg, 3 Eq, 2.26 mmol) in DMF (12 mL) were added DIPEA (146 mg, 1.5 Eq, 1.13 mmol) and Ac₂0 (231 mg, 3 Eq, 2.26 mmol) and Pd(OAc)₂ (17 mg, 0.1 Eq, 75 μmol) and XantPhos (87 mg, 0.2 Eq, 0.15 mmol) at rt under nitrogen atmosphere. The resulting mixture was stirred for 4 h at 100° C. under nitrogen atmosphere. The mixture was allowed to cool down to rt. The crude product was purified by reverse flash column chromatography with the following conditions: Column, C18; mobile phase, water (0.1% FA) and MeCN (10% MeCN up to 30% in 10 min); Detector, UV 254/220 nm. This resulted in the sub-title compound (AEJ-2) (150 mg, 0.44 mmol, 58%, 90% Purity) as a yellow solid. m/z 342.1 (M+H)⁺ (ES+).

Step 3: 6-(Ethylamino)-4-[4-fluoro-2-(4-methyl-1,2,4-triazol-3-yl)phenyl]-N-[2-hydroxy-3-(trifluoromethyl)phenyl]pyridine-2-carboxamide (AEJ-3). To a stirred mixture of the product from step 2 above (AEJ-2) (130 mg, 1 Eq, 0.38 mmol) and 2-amino-6-(trifluoromethyl)phenol (K-1) (202 mg, 3 Eq, 1.14 mmol) in MeCN (5 mL) were added TCFH (321 mg, 3 Eq, 1.14 mmol) and NMI (313 mg, 10 Eq, 3.81 mmol) at 0° C. The resulting mixture was stirred for 2 h at rt under nitrogen atmosphere. The mixture was concentrated in vacuo. The crude product was purified by reverse flash column chromatography with the following conditions: Column, C18; mobile phase, water (0.1% FA) and MeCN (36% MeCN up to 48% in 10 min); Detector, UV 254/220 nm. The product-containing fractions were combined and concentrated in vacuo. This resulted in the sub-title compound (AEJ-3) (140 mg, 0.28 mmol, 73%, 92% Purity) as a white solid. m/z 501.2 (M+H)⁺ (ES+).

Step 4: N-Ethyl-4-[4-fluoro-2-(4-methyl-1,2,4-triazol-3-yl)phenyl]-6-[7-(trifluoromethyl)-1,3-benzoxazol-2-yl]pyridin-2-amine (AEJ-4). To a stirred solution of the product from step 3 above (AEJ-3) (40 mg, 1 Eq, 82 μmol) in xylene (3 mL) was added TsOH (14 mg, 1 Eq, 82 μmol) at rt. The resulting mixture was stirred for overnight at 140° C. under nitrogen atmosphere. The mixture was cooled to rt and concentrated in vacuo. The crude product was purified by reverse flash column chromatography with the following conditions: Column, C18; mobile phase, water (0.1% NH₄HCO₃) and MeCN (20% MeCN up to 60% in 10 min); Detector, UV 254/220 nm. The product-containing fractions were combined and concentrated in vacuo. The crude product was purified by Prep-HPLC with the following conditions: Column: SunFire Prep C18 OBD Column, 19×150 mm, 5 μm; Mobile Phase A: water (0.1% NH₄HCO₃+0.1% NH₃·H₂O), Mobile Phase B: MeCN; Flow rate: 60 mL/min; Gradient: 30 B to 60 B in 7 min; Detector, UV 254/210 nm; RT: 6.08. This resulted in the title compound (AEJ-4) (3.4 mg, 7.0 μmol, 8.4%, 98% Purity) as a white solid. m/z 483.2 (M+H)⁺ (ES+). ¹H NMR (400 MHz, MeOH-d4) δ 8.45 (s, 1H), 8.03 (d, J=8.0 Hz, 1H), 7.83-7.73 (m, 2H), 7.63-7.43 (m, 3H), 7.31 (d, J=1.4 Hz, 1H), 6.40 (d, J=1.3 Hz, 1H), 3.34 (d, J=19.2 Hz, 5H), 1.23 (t, J=7.2 Hz, 3H).

### Example 322: {2-[6-(Ethylamino)-4-[4-fluoro-2-(4-methyl-1,2,4-triazol-3-yl)-phenyl]-pyridin-2-yl]-7-(trifluoromethyl)-1,3-benzoxazol-5-yl}-methanol (AEK-2)

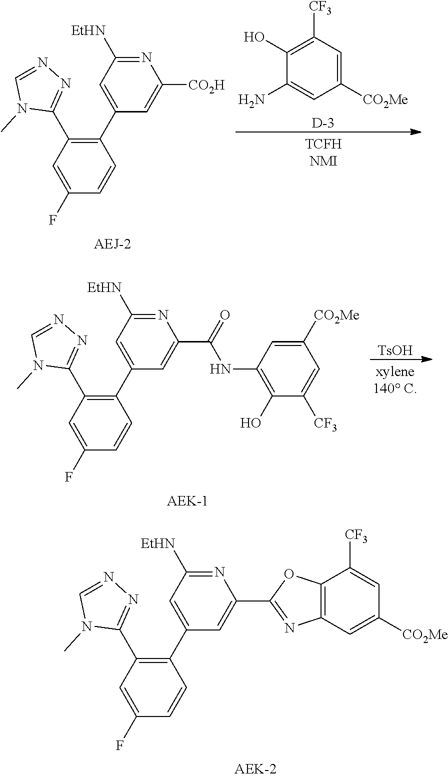

Step 1: Methyl 3-[6-(ethylamino)-4-[4-fluoro-2-(4-methyl-1,2,4-triazol-3-yl)-phenyl]-pyridine-2-amido]-4-hydroxy-5-(trifluoromethyl)-benzoate (AEK-1). To a stirred solution of intermediate (AEJ-2) (50 mg, 1 Eq, 0.14 mmol) and intermediate (D-3) (41 mg, 1.2 Eq, 0.17 mmol) in MeCN (8 mL) were added TCFH (123 mg, 3 Eq, 0.43 mmol) and NMI (120 mg, 10 Eq, 1.46 mmol) at 0° C. The resulting mixture was stirred for 1 h at rt. The mixture was concentrated in vacuo. The crude product was purified by reverse flash column chromatography with the following conditions: Column, C18; mobile phase, water (0.1% FA) and MeCN (60% MeCN up to 70% in 10 min); Detector, UV 254/220 nm. The product-containing fractions were combined and concentrated in vacuo. This resulted in the sub-title compound (AEK-1) (50 mg, 0.09 mmol, 61%, 90% Purity) as a white solid. m/z 559.2 (M+H)⁺ (ES+).

Step 2: Methyl 2-[6-(ethylamino)-4-[4-fluoro-2-(4-methyl-1,2,4-triazol-3-yl)-phenyl]-pyridin-2-yl]-7-(trifluoromethyl)-1,3-benzoxazole-5-carboxylate (AEK-2). A solution of the product from step 1 above (AEK-1) (30 mg, 1 Eq, 0.05 mmol) and TsOH (28 mg, 3 Eq, 0.16 mmol) in xylene (5 mL) was stirred for overnight at 140° C. The mixture was cooled to rt and concentrated in vacuo. The crude product was purified by reverse flash column chromatography with the following conditions: Column, C18; mobile phase, water (0.1% FA) and MeCN (50% MeCN up to 60% in 10 min); Detector, UV 254/220 nm. The crude product was purified by Prep-HPLC with the following conditions: Column: SunFire Prep C18 OBD Column, 20×250 mm, 5 μm; Mobile Phase A: water (0.1% NH₄HCO₃+0.1% NH₃·H₂O), Mobile Phase B: MeCN; Flow rate: 60 mL/min; Gradient: 40 B to 60 B in 10 min; Detector, UV 254/210 nm; RT: 9.78. The product-containing fractions were combined and evaporated partially in vacuo and lyophilized overnight to afford the title compound (AEK-2) (3.2 mg, 5.9 μmol, 11%, 96% Purity) as an off-white solid. m/z 541.2 (M+H)⁺ (ES+). ¹H NMR (300 MHz, MeOH-d4) δ 8.63 (s, 1H), 8.45 (s, 1H), 8.36 (s, 1H), 7.81-7.75 (m, 1H), 7.57-7.41 (m, 2H), 7.34 (d, J=1.4 Hz, 1H), 6.43 (d, J=1.4 Hz, 1H), 4.00 (s, 3H), 3.35 (d, J=5.7 Hz, 5H), 1.23 (t, J=7.2 Hz, 3H).

### Example 323: {2-[6-(Ethylamino)-4-[4-fluoro-2-(4-methyl-1,2,4-triazol-3-yl)-phenyl]-pyridin-2-yl]-7-(trifluoromethyl)-1,3-benzoxazol-5-yl}-methanol (AEL-1)

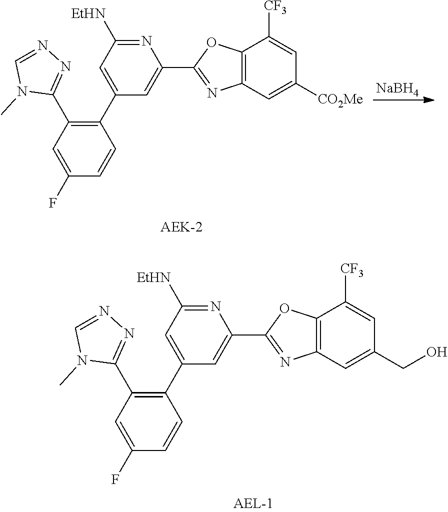

A solution of compound (AEK-2) (20 mg, 1 Eq, 0.03 mmol) and NaBH₄ (70 mg, 50 Eq, 1.85 mmol) in MeOH (5 mL) was stirred for 1 h at rt. The resulting mixture was concentrated in vacuo. The crude product was purified by reverse flash column chromatography with the following conditions: Column, C18; mobile phase, water (0.1% NH₄HCO₃) and MeCN (40% MeCN up to 60% in 10 min); Detector, UV 254/220 nm. The product-containing fractions were combined and concentrated in vacuo. The crude product was purified by Prep-HPLC with the following conditions: Column: SunFire Prep C18 OBD Column, 20×250 mm 5 μm; Mobile Phase A: water (0.1% NH₄HCO₃+0.1% NH₃·H₂O), Mobile Phase B: MeCN; Flow rate: 60 mL/min; Gradient: 45 B to 55 B in 10 min; Detector, UV 254/210 nm; RT: 9.78. The product-containing fractions were combined and evaporated partially in vacuo and lyophilized overnight to afford the title compound (AEL-1) (1.6 mg, 3.1 μmol, 8%, 98% Purity) as an off-white solid. m/z 513.2 (M+H)⁺ (ES+) 1H NMR (300 MHz, Methanol-d4) δ 8.45 (s, 1H), 8.00 (s, 1H), 7.85-7.72 (m, 2H), 7.59-7.43 (m, 2H), 7.31 (d, J=1.4 Hz, 1H), 6.40 (d, J=1.4 Hz, 1H), 4.79 (s, 2H), 3.36 (s, 4H), 3.34 (s, 1H), 1.31-1.18 (m, 3H).

### Example 324: (1S,2R)-2-(((7-Chloro-2-(4′-fluoro-2′-(4-methyl-4H-1,2,4-triazol-3-yl)-[1,1′-biphenyl]-3-yl)benzo[d]oxazol-5-yl)methyl)amino)cyclopentan-1-ol (HG-7)

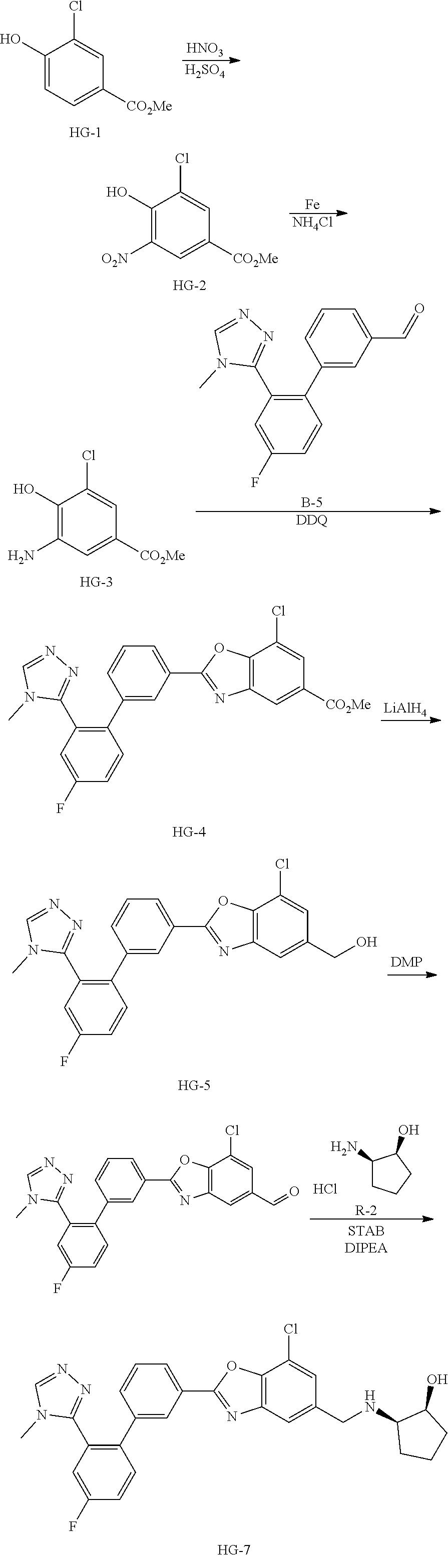

Step 1: Methyl 3-chloro-4-hydroxy-5-nitrobenzoate (HG-2). To a mixture of methyl 3-chloro-4-hydroxybenzoate (HG-1) (1.5 g, 1 Eq, 8.0 mmol) suspended in conc. H₂SO₄ (28 g, 15 mL, 35 Eq, 0.28 mol) was added HNO₃ (1.3 g, 0.90 mL, 2.5 Eq, 20 mmol) slowly at 0° C. The resulting suspension was stirred at this temperature for 15 min. The mixture was poured into ice water (250 mL). A precipitation was observed, the product was filtered, washed, and dried to afford the sub-title compound (HG-2) (1.3 g, 5.3 mmol, 66%, 95% Purity) as a yellow solid. ¹H NMR (400 MHz, CDCl₃) δ 11.35 (s, 1H), 8.74 (d, J=2.0 Hz, 1H), 8.35 (d, J=2.1 Hz, 1H), 3.96 (s, 3H).

Step 2: Methyl 3-amino-5-chloro-4-hydroxybenzoate (HG-3). To a stirred solution of the product from step 1 above (HG-2) (500 mg, 1 Eq, 2.16 mmol) in dry EtOH (4 mL) under a nitrogen atmosphere at 80° C. was added Fe powder (844 mg, 7 Eq, 15.1 mmol) and NH₄Cl (1.15 g) in H₂O (3 mL). The reaction mixture was stirred for 3 h at 55° C. After completion of the reaction, the crude was passed through the celite pad to remove metallic Fe and immediately transferred the celite waste into water. The filtrate was dried and concentrated in vacuo. The crude product was purified by chromatography on silica gel (40 g cartridge, 25-100% EtOAc/isohexane) to afford the sub-title compound (HG-3) (160 mg, 0.78 mmol, 36%, 98% Purity) as a light-yellow solid. m/z 202.1 (M+H)⁺ (ES+). ¹H NMR (400 MHz, CDCl3) δ 7.47 (s, 1H), 7.32 (s, 1H), 5.78 (s, 1H), 3.94 (s, 2H), 3.87 (s, 3H).

Step 3: Methyl 7-chloro-2-(4′-fluoro-2′-(4-methyl-4H-1,2,4-triazol-3-yl)-[1,1′-biphenyl]-3-yl)benzo[d]oxazole-5-carboxylate (HG-4). To a solution of intermediate (B-5) (209 mg, 1 Eq, 744 μmol) in MeOH (10 mL) was added the product from step 2 above (HG-3) (150 mg, 1 Eq, 744 μmol). The resulting mixture was stirred at 45° C. for 16 h. After concentration in vacuo, the residue was dissolved in DCM (10 mL) and DDQ (186 mg, 1.1 Eq, 818 μmol) was then added. After stirring at rt for 1 h, the resulting mixture was diluted with DCM (10 mL) and washed sequentially with sat. aq. sol. of NaHCO₃ (2×25 mL) and brine (10 mL). The organic layer was dried (Na₂SO₄) and the solvent was removed in vacuo. The crude product was purified by chromatography on silica gel (80 g cartridge, 0-10% MeOH/DCM) to afford the sub-title compound (HG-4) (281 mg, 592 μmol, 79.6%, 97.5% Purity) as a dark-brown powder. m/z 463.1 (M+H)⁺ (ES+).

Step 4: (7-Chloro-2-(4′-fluoro-2′-(4-methyl-4H-1,2,4-triazol-3-yl)-[1,1′-biphenyl]-3-yl)benzo[d]oxazol-5-yl)methanol (HG-5). To a solution of the product from step 3 above (HG-4) (260 mg, 1 Eq, 562 μmol) in THF (20 mL) was added LiAlH₄ (25.6 mg, 674 μL, 1 molar, 1.2 Eq, 674 μmol) dropwise at −20° C. This mixture was stirred 1 h at this temperature. The reaction mixture was diluted with distilled water (25 mL) and acidified with 20 mL 1M HCl and transferred into a separating funnel. The aq. layer was extracted with DCM (3×25 mL). The combined extracted layers were collected, dried (Na₂SO₄), filtered and concentrated in vacuo. The crude product was purified by chromatography on silica gel (40 g cartridge, 0-10% (0.7 M ammonia/MeOH)/DCM) to afford the sub-title compound (HG-5) (200 mg, 0.45 mmol, 80%, 98% Purity) as a pale-brown Form. m/z 435.9 (M+H)⁺ (ES+). ¹H NMR (400 MHz, DMSO) δ=8.43 (s, 1H), 8.13 (dt, J=8.0, 1.3 Hz, 1H), 7.96 (t, J=1.8 Hz, 1H), 7.79 (dd, J=8.6, 5.6 Hz, 1H), 7.70 (d, J=1.2 Hz, 1H), 7.64-7.47 (m, 4H), 7.32 (ddd, J=7.8, 1.9, 1.1 Hz, 1H), 5.45 (t, J=5.8 Hz, 1H), 4.62 (d, J=5.8 Hz, 2H), 3.18 (s, 3H).

Step 5: 7-Chloro-2-(4′-fluoro-2′-(4-methyl-4H-1,2,4-triazol-3-yl)-[1,1′-biphenyl]-3-yl)benzo[d]oxazole-5-carbaldehyde (HG-6). To a stirred solution of the product from step 4 above (HG-5) (50 mg, 1 Eq, 0.11 mmol) in dry DCM (20 mL) under a nitrogen atmosphere at 25° C. was added DMP (98 mg, 2 Eq, 0.23 mmol). The reaction mixture was stirred for 1 h. The reaction mixture was diluted with DCM (10 mL) and washed with sat. aq. NaHCO₃ (10 mL) and brine. Organics were separated, dried (MgSO₄), filtered and evaporated in vacuo. The crude product was purified by chromatography on silica gel (40 g cartridge, 0-10% (0.7 M ammonia/MeOH)/DCM) to afford the sub-title compound (HG-6) (47 mg, 98 μmol, 85%, 90% Purity) as a pale-yellow solid.

Step 6: (1S,2R)-2-(((7-Chloro-2-(4′-fluoro-2′-(4-methyl-4H-1,2,4-triazol-3-yl)-[1,1′-biphenyl]-3-yl)benzo[d]oxazol-5-yl)methyl)amino)cyclopentan-1-ol (HG-7). A solution of the product from step 5 above (HG-6) (50 mg, 1 Eq, 0.12 mmol), (1S,2R)-2-(chloro-15-azaneyl)cyclopentan-1-ol, HCl (R-2) (32 mg, 2 Eq, 0.23 mmol) and DIPEA (45 mg, 60 μL, 3 Eq, 0.35 mmol) in CHCl₃ (10 mL) was stirred for 12 h at rt. Then NaBH(OAc)₃ (98 mg, 4 Eq, 0.46 mmol) was added and the reaction mixture was stirred at rt for another 12 h. The reaction mixture was diluted with NaHCO₃ (25 mL) and transferred into a separating funnel. The layer was extracted with EtOAc (3×25 mL). The combined extracted layers were collected, dried (Na₂SO₄), filtered and concentrated in vacuo. The crude product was purified by chromatography on silica gel (40 g cartridge, 0-10% MeOH/DCM) to afford the title compound (HG-7) (35 mg, 66 μmol, 57%, 98% Purity) as a clear white solid. m/z 518.1 (M+H)⁺ (ES+). ¹H NMR (400 MHz, DMSO) δ=8.43 (s, 1H), 8.19-8.11 (m, 2H), 7.98 (t, J=1.8 Hz, 1H), 7.93 (d, J=1.5 Hz, 1H), 7.78 (dd, J=8.6, 5.6 Hz, 1H), 7.63-7.52 (m, 3H), 7.37-7.30 (m, 1H), 4.38 (s, 1H), 3.94 (dt, J=6.6, 4.0 Hz, 1H), 3.92-3.79 (m, 2H), 3.30 (s, 1H), 3.19 (s, 3H), 2.76 (ddd, J=8.7, 6.8, 4.1 Hz, 1H), 1.74-1.51 (m, 4H), 1.42 (dtd, J=9.3, 7.3, 5.0 Hz, 2H).

### Example 325: 2-(4′-Fluoro-2′-(4-methyl-4H-1,2,4-triazol-3-yl)-[1,1′-biphenyl]-3-yl)-5-((((1R,2S)-2-hydroxycyclopentyl)amino)methyl)benzo[d]oxazole-7-carbonitrile (HH-3)

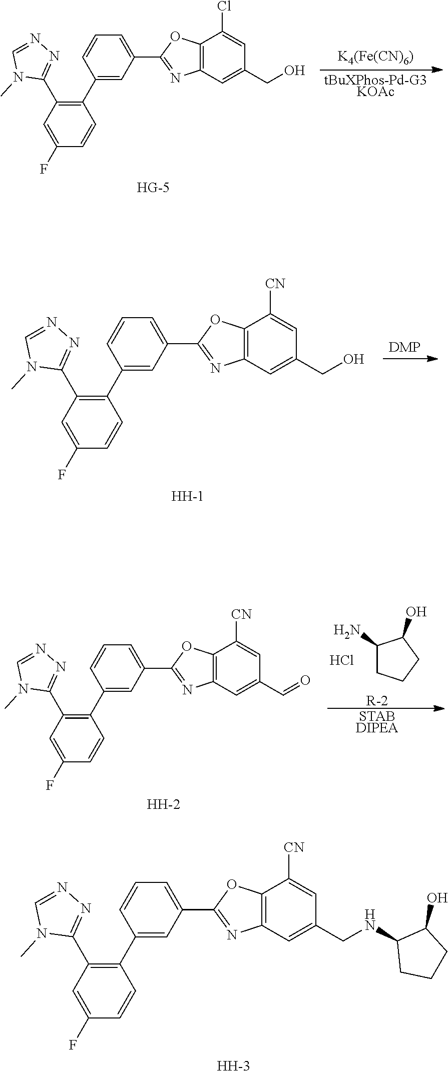

Step 1: 2-(4′-Fluoro-2′-(4-methyl-4H-1,2,4-triazol-3-yl)-[1,1′-biphenyl]-3-yl)-5-(hydroxymethyl)benzo[d]oxazole-7-carbonitrile (HH-1). Intermediate (HG-5) (50 mg, 1 Eq, 0.11 mmol) and potassium acetate (23 mg, 2 Eq, 0.23 mmol) were suspended in a mixture of 1,4-dioxane (2 mL) and water (0.4 mL). The mixture was degassed via 3 vacuum/nitrogen cycles before tBuXPhos-G3-Pd (9.1 mg, 0.1 Eq, 11 μmol) and K₄Fe(CN)₆ (1.5 Eq, 73 mg, 0.17 mmol) were added. The mixture was further degassed via another 3 vacuum/nitrogen cycles before being heated to 105° C. for 3 h. The reaction was allowed to cool to rt and diluted with EtOAc (200 mL). The mixture was dried (Na₂SO₄), filtered and concentrated in vacuo. The crude product was loaded on to silica (˜5 g) and purified by chromatography on silica gel (40 g cartridge, 0-100% EtOAc/isohexane) to the sub-title compound (HH-1) (30 mg, 65 μmol, 56%, 92% Purity) as a clear white solid.

Step 2: 2-(4′-Fluoro-2′-(4-methyl-4H-1,2,4-triazol-3-yl)-[1,1′-biphenyl]-3-yl)-5-formylbenzo[d]oxazole-7-carbonitrile (HH-2). To a stirred solution of the product from step 1 above (HH-1) (30 mg, 1 Eq, 71 μmol) in dry DCM (20 mL) under a nitrogen atmosphere at 25° C. was added DMP (60 mg, 2 Eq, 0.14 mmol). The reaction mixture was stirred for 1 h. The reaction mixture was diluted with DCM (10 mL) and washed with sat. aq. NaHCO₃ (10 mL) and brine. Organics were separated, dried (MgSO₄), filtered and evaporated in vacuo. The crude product was purified by chromatography on silica gel (40 g cartridge, 0-10% (0.7 M ammonia/MeOH)/DCM) to afford the sub-title compound (HH-2) (27 mg, 61 μmol, 87%, 96% Purity) as a pale-yellow solid. m/z 424.1 (M+H)⁺ (ES+).

Step 3: 2-(4′-Fluoro-2′-(4-methyl-4H-1,2,4-triazol-3-yl)-[1,1′-biphenyl]-3-yl)-5-((((1R,2S)-2-hydroxycyclopentyl)amino)methyl)benzo[d]oxazole-7-carbonitrile (HH-3). A solution of the product from step 2 above (HH-2) (30 mg, 1 Eq, 71 μmol), (1S,2R)-2-(chloro-15-azaneyl)cyclopentan-1-ol, HCl (R-2) (20 mg, 2 Eq, 0.14 mmol) and DIPEA (27 mg, 37 μL, 3 Eq, 0.21 mmol) in CHCl₃ (10 mL) was stirred for 12 h at rt. Then NaBH(OAc)₃ (60 mg, 4 Eq, 0.28 mmol) was added and the reaction mixture was stirred at rt for another 12 h. The reaction mixture was diluted with NaHCO₃ (25 mL) and transferred into a separating funnel. The layer was extracted with EtOAc (3×25 mL). The combined extracted layers were collected, dried (Na₂SO₄), filtered and concentrated in vacuo. The crude product was purified by chromatography on silica gel (40 g cartridge, 0-10% MeOH/DCM) to afford the title compound (HH-3) (20 mg, 39 μmol, 54%, 98% Purity) as a clear white powder. m/z 509.2 (M+H)⁺ (ES+). ¹H NMR (400 MHz, MeOD) δ 8.43 (s, 1H), 8.26 (dt, J=8.0, 1.3 Hz, 1H), 8.16-8.07 (m, 2H), 7.86 (d, J=1.6 Hz, 1H), 7.82 (dd, J=8.6, 5.4 Hz, 1H), 7.59 (dt, J=11.7, 8.2 Hz, 2H), 7.53-7.43 (m, 2H), 4.21-4.14 (m, 1H), 4.07-3.92 (m, 2H), 3.30 (s, 3H), 2.98 (td, J=8.3, 4.2 Hz, 1H), 1.99-1.70 (m, 4H), 1.58 (dq, J=17.5, 8.7 Hz, 2H). Two exchangeable protons (NH and OH) not observed.

### Example 326: (1R,2S)-2-(((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)amino)cyclopentan-1-ol (HI-2)

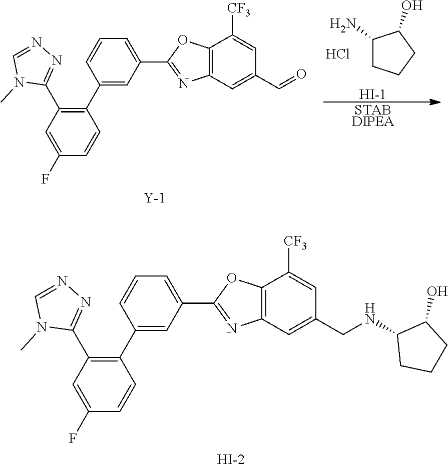

A solution of intermediate (Y-1) (30.4 mg, 1 Eq, 65.2 μmol), (1R,2S)-2-aminocyclopentanol, HCl (HI-1) (17.3 mg, 1.93 Eq, 126 μmol) and DIPEA (41 mg, 55 μL, 4.8 Eq, 0.32 mmol) in CHCl₃ (2.0 mL) was stirred 1 h at rt. NaBH(OAc)₃ (40.6 mg, 97% Wt, 2.85 Eq, 186 μmol) was added and the reaction mixture was stirred at rt overnight. NaBH(OAc)₃ (40.5 mg, 97% Wt, 2.84 Eq, 185 μmol) was added and the resulting mixture was stirred at rt for 3 h. The reaction mixture was diluted with EtOAc (5 mL), washed with sat. NaHCO₃ (5 mL) and brine (5 mL), dried (Na₂SO₄), filtered and concentrated in vacuo. The crude product was purified by chromatography on silica gel (4 g cartridge, 0-10% MeOH/DCM) to afford the title compound (HI-2) (16.1 mg, 29 μmol, 44%, 99% Purity) as a white solid. m/z 552.2 (M+H)⁺ (ES+). ¹H NMR (400 MHz, DMSO) δ 8.42 (s, 1H), 8.15-8.11 (m, 1H), 8.09 (s, 1H), 7.91 (d, J=1.9 Hz, 1H), 7.81-7.75 (m, 2H), 7.64-7.53 (m, 3H), 7.38 (d, J=7.9 Hz, 1H), 3.99-3.91 (m, 2H), 3.88 (d, J=13.9 Hz, 1H), 3.20 (s, 3H), 2.82-2.74 (m, 1H), 1.72-1.56 (m, 4H), 1.46-1.37 (m, 2H). 2× exchangeable protons not observed.

### Example 327: (1S,2S)-2-(((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)amino)cyclopentan-1-ol (HJ-1)

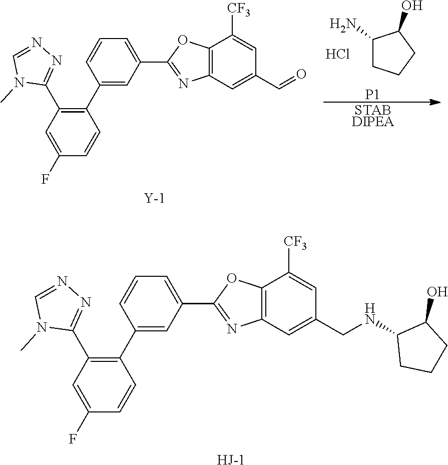

A solution of intermediate (Y-1) (31.3 mg, 98% Wt, 1 Eq, 65.8 μmol), (1S,2S)-2-aminocyclopentan-1-ol, HCl (P-1) (18.9 mg, 2.09 Eq, 137 μmol) and DIPEA (41 mg, 55 μL, 4.8 Eq, 0.32 mmol) in CHCl₃ (2.0 mL) was stirred 1 h at rt. NaBH(OAc)₃ (42.6 mg, 97% Wt, 2.96 Eq, 195 μmol) was added and the reaction mixture was stirred at rt overnight. NaBH(OAc)₃ (44.7 mg, 97% Wt, 3.11 Eq, 205 μmol) was added and the resulting mixture was stirred at rt for 3 h. The reaction mixture was diluted with EtOAc (5 mL), washed with sat. NaHCO₃ (5 mL) and brine (5 mL), dried (Na₂SO₄), filtered and concentrated in vacuo. The crude product was purified by chromatography on silica gel (4 g cartridge, 0-10% MeOH/DCM) to afford the title compound (HJ-1) (18.4 mg, 33 μmol, 50%, 99% Purity) as a white solid. m/z 552.2 (M+H)⁺ (ES+). ¹H NMR (400 MHz, DMSO) δ 8.42 (s, 1H), 8.15-8.11 (m, 1H), 8.09-8.06 (m, 1H), 7.92-7.89 (m, 1H), 7.80-7.75 (m, 2H), 7.63-7.53 (m, 3H), 7.40-7.36 (m, 1H), 4.50 (d, J=4.1 Hz, 1H), 3.93-3.86 (m, 2H), 3.83-3.78 (m, 1H), 3.20 (s, 3H), 1.87-1.78 (m, 2H), 1.62-1.52 (m, 2H), 1.45-1.36 (m, 1H), 1.35-1.26 (m, 1H). 2× exchangeable protons not observed.

### Example 328: 1-(2-(4′-Fluoro-2′-(4-methyl-4H-1,2,4-triazol-3-yl)-[1,1′-biphenyl]-3-yl)-7-(trifluoromethyl)benzo[d]oxazol-5-yl)-N-((1-methylcyclobutyl)methyl)methanamine (HK-2)

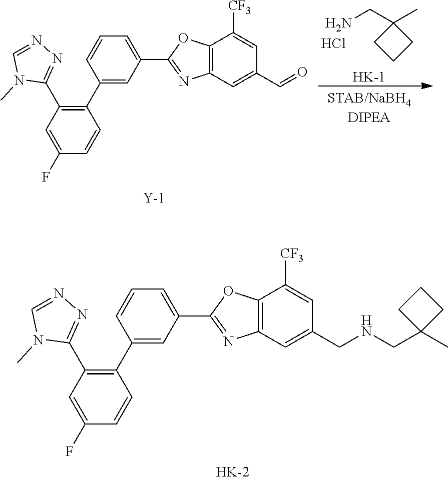

NaBH(OAc)₃ (36 mg, 3.0 Eq, 0.17 mmol) was added to a stirred mixture of intermediate (Y-1) (35 mg, 76% Wt, 1 Eq, 57 μmol), 1-methyl-cyclobutanemethanamine, HCl (HK-1) (15 mg, 2.0 Eq, 0.11 mmol) and DIPEA (22 mg, 30 μL, 3.0 Eq, 0.17 mmol) in CHCl₃ (3 mL). The reaction mixture was stirred at 40° C. overnight. NaBH₄ (22 mg, 10.0 Eq, 0.57 mmol) was added to the reaction mixture and stirring continued at 40° C. for an additional 4 h. The reaction mixture was allowed to stir at rt over the weekend. The reaction mixture was diluted with DCM (4 mL) and washed with sat. aq. NaHCO₃ (10 mL). The organic phase was separated and evaporated to afford the crude product. The crude was purified by preparative HPLC (Waters, Basic (0.3% ammonia), Waters XBridge BEH C18 ODB, 5 μm, 30×100 mm column 55-100% MeCN in water) to afford the title compound (HK-2) (10 mg, 17 μmol, 30%, 94% Purity) as a white solid. m/z 550.4 (M+H)⁺ (ES+). ¹H NMR (400 MHz, DMSO) δ 8.42 (s, 1H), 8.13 (dt, J=7.9, 1.4 Hz, 1H), 8.08 (s, 1H), 7.91 (t, J=1.8 Hz, 1H), 7.83-7.73 (m, 2H), 7.65-7.51 (m, 3H), 7.37 (dt, J=7.8, 1.4 Hz, 1H), 3.91 (s, 2H), 3.29 (s, 2H), 3.20 (s, 3H), 2.41 (s, 1H), 1.88-1.69 (m, 4H), 1.62-1.54 (m, 2H), 1.11 (s, 3H).

### Example 329: (R)-1-(2-(4′-Fluoro-2′-(4-methyl-4H-1,2,4-triazol-3-yl)-[1,1′-biphenyl]-3-yl)-7-(trifluoromethyl)benzo[d]oxazol-5-yl)-N-((2-methyloxetan-2-yl)methyl)methanamine (HL-2)

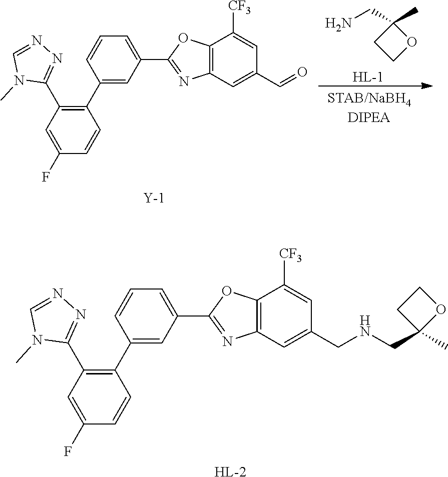

NaBH(OAc)₃ (36 mg, 3.0 Eq, 0.17 mmol) was added to a stirred mixture of intermediate (Y-1) (35 mg, 76% Wt, 1 Eq, 57 μmol), (R)-(2-methyloxetan-2-yl)methanamine (HL-1) (12 mg, 2.0 Eq, 0.11 mmol) and DIPEA (22 mg, 30 μL, 3.0 Eq, 0.17 mmol) in CHCl₃ (3 mL). The reaction mixture was stirred at 40° C. overnight. NaBH₄ (22 mg, 10.0 Eq, 0.57 mmol) was added to the reaction mixture and stirring continued at 40° C. for an additional 4 h. The reaction mixture was allowed to stir at rt over the weekend. The reaction mixture was diluted with DCM (4 mL) and washed with sat. aq. NaHCO₃ (10 mL). The organic phase was separated and evaporated to afford the crude product. The crude was purified by preparative HPLC (Waters, Basic (0.3% ammonia), Waters XBridge BEH C18 ODB, 5 μm, 30×100 mm column 35-100% MeCN in water) to afford the title compound (HL-2) (11 mg, 19 μmol, 34%, 97% Purity) as a white solid. m/z 552.3 (M+H)⁺ (ES+). ¹H NMR (400 MHz, DMSO) δ 8.42 (s, 1H), 8.17-8.07 (m, 2H), 7.91 (t, J=1.8 Hz, 1H), 7.83-7.73 (m, 2H), 7.65-7.51 (m, 3H), 7.38 (dt, J=7.9, 1.4 Hz, 1H), 4.40-4.26 (m, 2H), 4.04-3.91 (m, 2H), 3.29 (s, 2H), 3.20 (s, 3H), 2.68-2.59 (m, 1H), 2.56 (s, 1H), 2.29-2.18 (m, 1H), 1.34 (s, 3H).

### Example 330: N-(2-Cyclopentylethyl)-2-(4′-fluoro-2′-(4-methyl-4H-1,2,4-triazol-3-yl)-[1,1′-biphenyl]-3-yl)-7-(trifluoromethyl)benzo[d]oxazol-5-amine (HM-3)

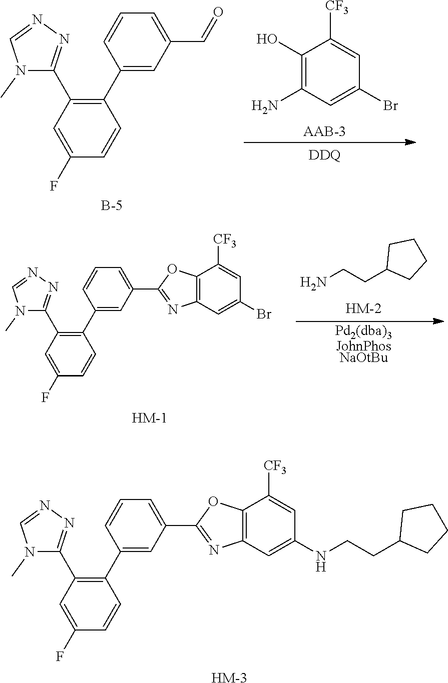

Step 1: 5-Bromo-2-(4′-fluoro-2′-(4-methyl-4H-1,2,4-triazol-3-yl)-[1,1′-biphenyl]-3-yl)-7-(trifluoromethyl)benzo[d]oxazole (HM-1). To a solution of intermediate (B-5) (325 mg, 1 Eq, 1.16 mmol) in MeOH (5 mL) was added 2-amino-4-bromo-6-(trifluoromethyl)phenol (AAB-3) (329 mg, 90% Wt, 1 Eq, 1.16 mmol). The resulting mixture was stirred at 45° C. for 16 h. After concentration in vacuo, the residue was dissolved in DCM (15 mL) and DDQ (289 mg, 1.1 Eq, 1.27 mmol) was then added. After stirring at rt for 1 h, the resulting mixture was diluted with DCM (10 mL) and washed sequentially with sat. aq. sol. of NaHCO₃ (2×25 mL) and brine (10 mL). The organic layer was dried (Na₂SO₄) and the solvent was removed n vacuo. The crude product was purified by chromatography on silica gel (80 g cartridge, 0-10% MeOH/DCM) to afford the sub-title compound (HM-1) (50 mg, 95 μmol, 8.2%, 98% Purity) as a light brown powder. m/z 518.3 (M+H)⁺ (ES+).

Step 2: N-(2-Cyclopentylethyl)-2-(4′-fluoro-2′-(4-methyl-4H-1,2,4-triazol-3-yl)-[1,1′-biphenyl]-3-yl)-7-(trifluoromethyl)benzo[d]oxazol-5-amine (HM-3). A stirred mixture of the product from step 1 above (HM-1) (34 mg, 1 Eq, 66 μmol), 2-cyclopentylethan-1-amine (HM-2) (15 mg, 17 μL, 2.0 Eq, 0.13 mmol), and NaOtBu (19 mg, 3 Eq, 0.20 mmol) and JohnPhos (3.9 mg, 0.2 eq, 66 μmol) in toluene (3 mL) was placed under three vacuum/N₂ cycles at 40° C. before Pd₂(dba)₃ (6.0 mg, 0.1 Eq, 6.6 μmol) was added. The mixture was degassed by another three vacuum/N₂ cycles before being warmed to 100° C. and stirring was continued for 3 h. The reaction mixture was concentrated in vacuo. The reaction mixture was diluted with distilled water (10 mL) and transferred into a separating funnel. The layer was extracted with DCM (3×10 mL). The combined extracted layers were collected, dried (Na₂SO₄), filtered and concentrated in vacuo. The crude product was purified by chromatography on silica gel (4 g cartridge, 0-10% MeOH/DCM) to afford the title compound (HM-3) (7 mg, 0.01 mmol, 20%, 99% Purity) as a pale-yellow solid. m/z 551.2 (M+H)⁺ (ES+). ¹H NMR (400 MHz, MeOD) δ 8.41 (s, 1H), 8.17 (dd, J=7.9, 1.5 Hz, 1H), 8.03 (t, J=1.7 Hz, 1H), 7.81 (dd, J=8.7, 5.5 Hz, 1H), 7.57 (td, J=8.2, 2.5 Hz, 2H), 7.49 (dd, J=8.8, 2.7 Hz, 1H), 7.45-7.40 (m, 1H), 7.05 (s, 1H), 7.00 (s, 1H), 4.61 (s, 1H), 3.26 (s, 3H), 3.19 (d, J=7.3 Hz, 2H), 1.99 (q, J=7.8 Hz, 1H), 1.90 (d, J=7.6 Hz, 2H), 1.72 (q, J=7.2 Hz, 3H), 1.66-1.56 (m, 2H), 1.23 (dd, J=12.0, 8.0 Hz, 2H). One exchangeable proton not observed.

### Example 331: 2-Cyclopentyl-N-((2-(4′-fluoro-2′-(4-methyl-4H-1,2,4-triazol-3-yl)-[1,1′-biphenyl]-3-yl)-7-(trifluoromethyl)benzo[d]oxazol-5-yl)methyl)ethan-1-amine (HN-1)

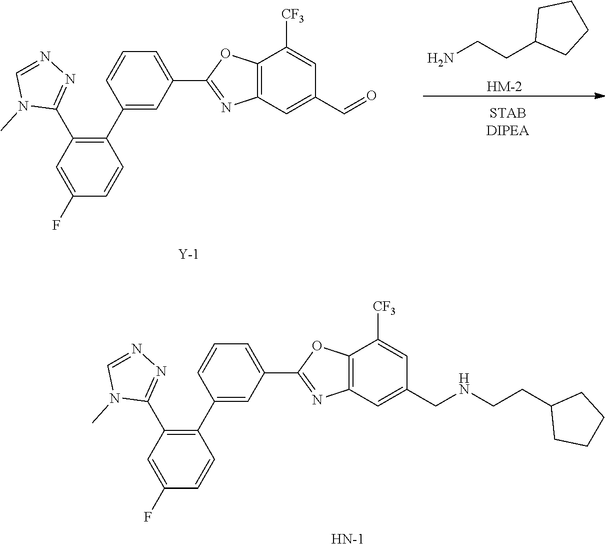

A solution of intermediate (Y-1) (30 mg, 1 Eq, 64 μmol), 2-cyclopentylethan-1-amine (HM-2) (15 mg, 17 μL, 2 Eq, 0.13 mmol) and DIPEA (25 mg, 34 μL, 3 Eq, 0.19 mmol) in CHCl₃ (10 mL) was stirred for 12 h at rt. NaBH(OAc)₃ (55 mg, 4 Eq, 0.26 mmol) was added and the reaction mixture was stirred at rt overnight. The reaction mixture was diluted with DCM (10 mL) and transferred into a separating funnel. The layer was extracted with DCM (3×10 mL). The combined extracted layers were collected, dried (Na₂SO₄), filtered and concentrated in vacuo. The crude product was purified by chromatography on silica gel (12 g cartridge, 0-10% MeOH/DCM) to afford the title compound (HN-1) (7 mg, 0.01 mmol, 20%, 98% Purity) as a pale-yellow solid. m/z 564.2 (M+H)⁺ (ES+). ¹H NMR (400 MHz, MeOD) δ 8.41 (s, 1H), 8.24 (dt, J=8.0, 1.3 Hz, 1H), 8.09 (t, J=1.8 Hz, 1H), 8.02 (s, 1H), 7.81 (dd, J=8.7, 5.4 Hz, 1H), 7.77 (s, 1H), 7.63-7.54 (m, 2H), 7.53-7.43 (m, 2H), 3.98 (s, 2H), 3.27 (s, 3H), 2.73-2.58 (m, 2H), 1.92-1.76 (m, 3H), 1.71-1.50 (m, 6H), 1.22-1.08 (m, 2H). One exchangeable proton not observed.

### Example 332: N-((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)cyclopropanamine (HO-2)

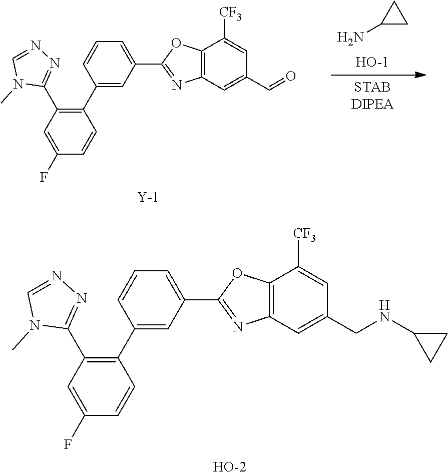

A solution of intermediate (Y-1) (30 mg, 1 Eq, 64 μmol), cyclopropanamine (HO-1) (7.3 mg, 8.9 μL, 2 Eq, 0.13 mmol) and DIPEA (25 mg, 34 μL, 3 Eq, 0.19 mmol) in CHCl₃ (10 mL) was stirred for 12 h at rt. NaBH(OAc)₃ (55 mg, 4 Eq, 0.26 mmol) was added and the reaction mixture was stirred at rt for another 12 h. The reaction mixture was diluted with brine (10 mL) and transferred into a separating funnel. The layer was extracted with DCM (3×10 mL). The combined extracted layers were collected, dried (Na₂SO₄), filtered and concentrated in vacuo. The crude product was purified by chromatography on silica gel (4 g cartridge, 0-10% MeOH/DCM) to afford the title compound (HO-2) (7 mg, 0.01 mmol, 20%, 98% Purity) as a clear colourless solid. m/z 508.2 (M+H)⁺ (ES+). ¹H NMR (400 MHz, MeOD) δ 8.41 (s, 1H), 8.24 (dt, J=8.0, 1.3 Hz, 1H), 8.09 (t, J=1.6 Hz, 1H), 8.02 (s, 1H), 7.81 (dd, J=8.6, 5.5 Hz, 1H), 7.76 (s, 1H), 7.64-7.53 (m, 2H), 7.53-7.42 (m, 2H), 4.02 (s, 2H), 3.27 (s, 3H), 2.23-2.13 (m, 1H), 0.55-0.47 (m, 2H), 0.44 (q, J=3.8 Hz, 2H). One exchangeable proton not observed.

### Example 333: 1-(1-Ethylcyclobutyl)-N-((2-(4′-fluoro-2′-(4-methyl-4H-1,2,4-triazol-3-yl)-[1,1′-biphenyl]-3-yl)-7-(trifluoromethyl)benzo[d]oxazol-5-yl)methyl)methanamine (HP-2)

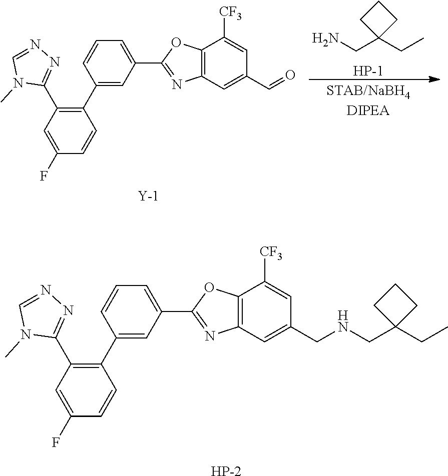

NaBH(OAc)₃ (36 mg, 3.0 Eq, 0.17 mmol) was added to a stirred mixture of intermediate (Y-1) (35 mg, 76% Wt, 1 Eq, 57 μmol), (1-ethylcyclobutyl)methanamine (HP-1) (13 mg, 2.0 Eq, 0.11 mmol) and DIPEA (22 mg, 30 μL, 3.0 Eq, 0.17 mmol) in CHCl₃ (3 mL). The reaction mixture was stirred at 40° C. overnight. NaBH₄ (22 mg, 10.0 Eq, 0.57 mmol) was added to the reaction mixture and stirring continued at 40° C. overnight. The reaction mixture was diluted with DCM (4 mL) and washed with sat. aq. NaHCO₃ (10 mL). The organic phase was separated and evaporated to afford the crude product. The crude was purified by preparative HPLC (Waters, Basic (0.3% ammonia), Waters XBridge BEH C18 ODB, 5 μm, 30×100 mm column 60-100% MeCN in water) to afford the title compound (HP-2) (10 mg, 17 μmol, 29%, 94% Purity) as a white foam. m/z 564.4 (M+H)⁺ (ES+). ¹H NMR (400 MHz, DMSO) δ 8.42 (s, 1H), 8.17-8.06 (m, 2H), 7.91 (t, J=1.8 Hz, 1H), 7.84-7.74 (m, 2H), 7.65-7.51 (m, 3H), 7.41-7.34 (m, 1H), 3.91 (s, 2H), 3.29 (s, 4H), 3.20 (s, 3H), 2.42 (s, 1H), 1.77-1.70 (m, 3H), 1.64-1.59 (m, 1H), 1.50 (q, J=7.4 Hz, 2H), 0.70 (t, J=7.4 Hz, 3H).

### Example 334: 1-(2-(4′-Fluoro-2′-(4-methyl-4H-1,2,4-triazol-3-yl)-[1,1′-biphenyl]-3-yl)-7-(trifluoromethyl)benzo[d]oxazol-5-yl)-N-((1-isobutylcyclobutyl)methyl)methanamine (HQ-2)

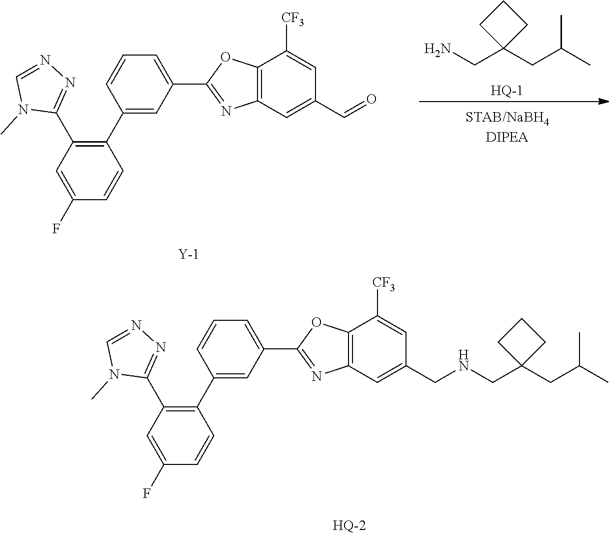

NaBH(OAc)₃ (36 mg, 3.0 Eq, 0.17 mmol) was added to a stirred mixture of intermediate (Y-1) (35 mg, 76% Wt, 1 Eq, 57 μmol), (1-isobutylcyclobutyl)methanamine (HQ-1) (16 mg, 2.0 Eq, 0.11 mmol) and DIPEA (22 mg, 30 μL, 3.0 Eq, 0.17 mmol) in CHCl₃ (3 mL). The reaction mixture was stirred at 40° C. overnight. NaBH₄ (22 mg, 10.0 Eq, 0.57 mmol) was added to the reaction mixture and stirring continued at 40° C. overnight. The reaction mixture was diluted with DCM (4 mL) and washed with sat. aq. NaHCO₃ (10 mL). The organic phase was separated and evaporated to afford the crude product. The crude was purified by preparative HPLC (Waters, Basic (0.3% ammonia), Waters XBridge BEH C18 ODB, 5 μm, 30×100 mm column 60-100% MeCN in water) to afford the title compound (HQ-2) (8 mg, 0.01 mmol, 20%, 97% Purity) as a white foam. m/z 592.5 (M+H)⁺ (ES+). ¹H NMR (400 MHz, DMSO) δ 8.41 (s, 1H), 8.13 (dt, J=7.9, 1.3 Hz, 1H), 8.07 (s, 1H), 7.91 (t, J=1.8 Hz, 1H), 7.82 (s, 1H), 7.78 (dd, J=8.7, 5.7 Hz, 1H), 7.65-7.51 (m, 3H), 7.37 (dt, J=8.0, 1.4 Hz, 1H), 3.92 (s, 2H), 3.29 (s, 4H), 3.20 (s, 3H), 2.14 (s, 1H), 1.81-1.68 (m, 3H), 1.56 (dt, J=13.3, 6.8 Hz, 1H), 1.39 (d, J=6.8 Hz, 2H), 0.80 (d, J=6.6 Hz, 6H). NH not observed.

### Example 335: 1-((((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)amino)methyl)cyclobutane-1-carbonitrile (HR-2)

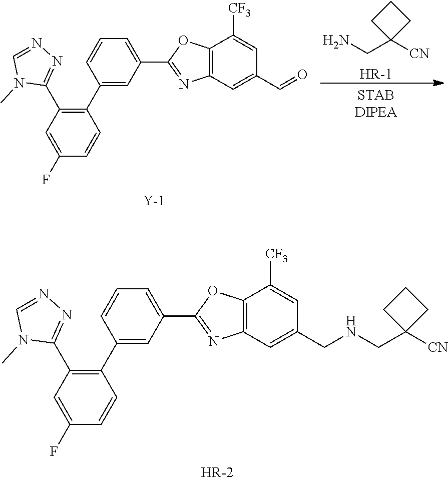

NaBH(OAc)₃ (36 mg, 3.0 Eq, 0.17 mmol) was added to a stirred mixture of intermediate (Y-1) (35 mg, 76% Wt, 1 Eq, 57 μmol), 1-(aminomethyl)cyclobutane-1-carbonitrile (HR-1) (13 mg, 2.0 Eq, 0.11 mmol) and DIPEA (22 mg, 30 μL, 3.0 Eq, 0.17 mmol) in CHCl₃ (3 mL). The reaction mixture was stirred at 40° C. overnight. More 1-(aminomethyl)cyclobutane-1-carbonitrile (HR-1) (13 mg, 2.0 Eq, 0.11 mmol) was added and stirring continued at 40° C. over the weekend. More NaBH(OAc)₃ (36 mg, 3.0 Eq, 0.17 mmol) was added and stirring continued at 40° C. overnight. The reaction mixture was diluted with DCM (4 mL) and washed with sat. aq. NaHCO₃ (10 mL). The organic phase was separated and evaporated to afford the crude product. The crude was purified by preparative HPLC (Waters, Basic (0.3% ammonia), Waters XBridge BEH C18 ODB, 5 μm, 30×100 mm column 40-100% MeCN in water) to afford the title compound (HR-2) (5 mg, 9 μmol, 20%, 100% Purity) as a colourless glass. m/z 561.7 (M+H)⁺ (ES+). ¹H NMR (400 MHz, MeOD) δ 8.41 (s, 1H), 8.24 (dt, J=7.9, 1.4 Hz, 1H), 8.12-8.03 (m, 2H), 7.85-7.77 (m, 2H), 7.65-7.54 (m, 2H), 7.53-7.43 (m, 2H), 4.07 (s, 2H), 3.38 (s, 1H), 3.28 (s, 3H), 2.94 (s, 2H), 2.55-2.42 (m, 2H), 2.26-2.02 (m, 4H).

### Example 336: (1S,2R)-2-(((7-Chloro-2-(4′-fluoro-2′-(4-methyl-4H-1,2,4-triazol-3-yl)-[1,1′-biphenyl]-3-yl)benzo[d]oxazol-5-yl)methyl)(methyl)amino)cyclopentan-1-ol (HS-3)

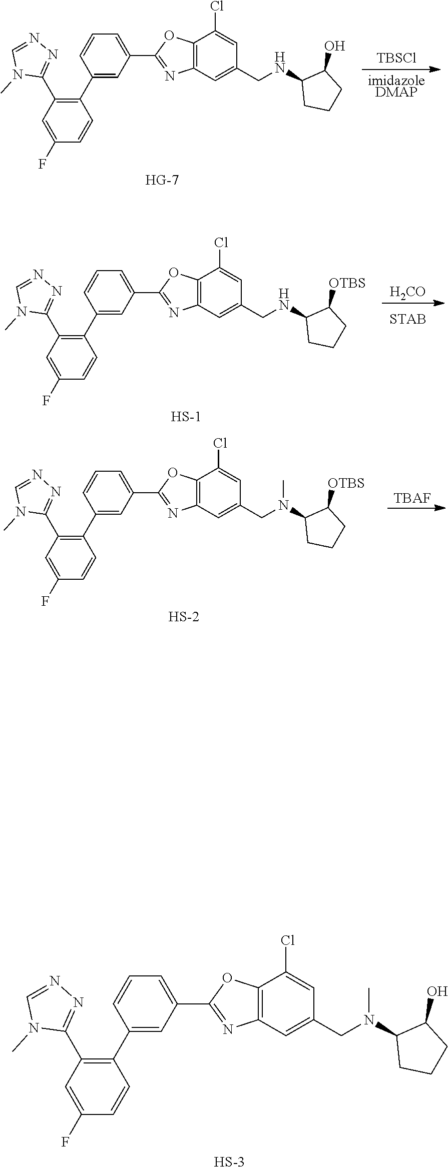

Step 1: (1R,2S)-2-((tert-Butyldimethylsilyl)oxy)-N-((7-chloro-2-(4′-fluoro-2′-(4-methyl-4H-1,2,4-triazol-3-yl)-[1,1′-biphenyl]-3-yl)benzo[d]oxazol-5-yl)methyl)cyclopentan-1-amine (HS-1). To a stirred solution of compound (HG-7) (70 mg, 1 Eq, 0.14 mmol), imidazole (46 mg, 5 Eq, 0.68 mmol) and DMAP (3.3 mg, 0.2 Eq, 27 μmol) in dry DMF (1 mL) under a nitrogen atmosphere at rt was added TBDMSCl (82 mg, 4.0 Eq, 0.54 mmol). The reaction mixture was stirred at rt for 4 h. The reaction mixture was diluted with distilled water (10 mL) and transferred into a separating funnel. The layer was extracted with EtOAc (3×10 mL). The combined extracted layers were collected, dried (Na₂SO₄), filtered and concentrated in vacuo. The crude product was purified by chromatography on silica gel (4 g cartridge, 0-10% MeOH/DCM) to afford the sub-title compound (HS-1) (60 mg, 91 μmol, 67%, 96% Purity) as a clear white gum. m/z 633.3 (M+H)⁺ (ES+). ¹H NMR (400 MHz, MeOD) δ=8.42 (s, 1H), 8.23 (dt, J=7.9, 1.3 Hz, 1H), 8.11 (t, J=1.8 Hz, 1H), 7.81 (dd, J=8.7, 5.4 Hz, 1H), 7.67 (d, J=1.4 Hz, 1H), 7.62-7.54 (m, 2H), 7.52-7.46 (m, 2H), 7.43 (ddd, J=7.8, 1.9, 1.1 Hz, 1H), 4.39-4.24 (m, 1H), 3.92 (s, 2H), 3.25 (s, 3H), 2.98 (td, J=7.5, 4.2 Hz, 1H), 1.99-1.70 (m, 4H), 1.59 (ddd, J=10.3, 7.9, 5.4 Hz, 2H), 0.94 (s, 9H), 0.15 (s, 6H). One exchangeable proton not observed.

Step 2: (1R,2S)-2-((tert-Butyldimethylsilyl)oxy)-N-((7-chloro-2-(4′-fluoro-2′-(4-methyl-4H-1,2,4-triazol-3-yl)-[1,1′-biphenyl]-3-yl)benzo[d]oxazol-5-yl)methyl)-N-methylcyclopentan-1-amine (HS-2). A solution of the product from step 1 above (HS-1) (60 mg, 96% Wt, 1 Eq, 91 μmol), formaldehyde solution in H₂O (22 mg, 20 μL, 37% Wt, 3 Eq, 0.27 mmol) and DIPEA (59 mg, 79 μL, 5 Eq, 0.46 mmol) in CHCl₃ (1 mL) was stirred at rt for 12 h. NaBH(OAc)₃ (97 mg, 5 Eq, 0.46 mmol) was added and the reaction mixture was stirred at rt overnight. The reaction mixture was diluted with DCM (2 mL), washed with sat. NaHCO₃ (2 mL) and brine (2 mL), dried (Na₂SO₄), filtered and concentrated in vacuo. The crude product was purified by chromatography on silica gel (4 g cartridge, 0-10% MeOH/DCM) to afford the sub-title compound (HS-2) (45 mg, 70 μmol, 76%, 100% purity) as a yellow oil. m/z 647.3 (M+H)⁺ (ES+).

Step 3: (1S,2R)-2-(((7-Chloro-2-(4′-fluoro-2′-(4-methyl-4H-1,2,4-triazol-3-yl)-[1,1′-biphenyl]-3-yl)benzo[d]oxazol-5-yl)methyl)(methyl)amino)cyclopentan-1-ol (HS-3). To a stirred solution of the product from step 2 above (HS-2) (51 mg, 1 Eq, 79 μmol) in dry THF (1.5 mL) under a nitrogen atmosphere at 25° C. was added TBAF in THF (72 mg, 0.28 mL, 1 molar, 3.5 Eq, 0.28 mmol). The reaction mixture was stirred for 4 h. A further 1.5 Eq. of TBAF solution was added and continued the stirring overnight. The reaction mixture was concentrated in vacuo. The reaction mixture was diluted with distilled water (5 mL) and transferred into a separating funnel. The layer was extracted with DCM (3×10 mL). The combined extracted layers were collected, dried (Na₂SO₄), filtered and concentrated in vacuo. The crude product was purified by chromatography on silica gel (4 g cartridge, 0-10% MeOH/DCM) to afford the title compound (HS-3) (35 mg, 64 μmol, 82%, 98% Purity) as a clear colourless solid. m/z 533.2 (M+H)⁺ (ES+). ¹H NMR (400 MHz, MeOD) δ 8.42 (s, 1H), 8.23 (dt, J=7.8, 1.3 Hz, 1H), 8.10 (t, J=1.7 Hz, 1H), 7.82 (dd, J=8.7, 5.5 Hz, 1H), 7.70 (d, J=1.4 Hz, 1H), 7.63-7.53 (m, 3H), 7.52-7.42 (m, 2H), 4.27 (d, J=3.9 Hz, 1H), 3.84 (d, J=13.3 Hz, 1H), 3.72 (d, J=13.2 Hz, 1H), 3.25 (s, 3H), 2.62 (s, 1H), 2.25 (s, 3H), 1.98 (d, J=9.0 Hz, 2H), 1.91-1.77 (m, 3H), 1.77-1.64 (m, 1H). One exchangeable proton not observed.

### Example 337: (1S,2R)-2-(((2-(4′-Fluoro-2′-(4-methyl-4H-1,2,4-triazol-3-yl)-[1,1′-biphenyl]-3-yl)-7-methoxybenzo[d]oxazol-5-yl)methyl)(methyl)amino)cyclopentan-1-ol (HT-3)

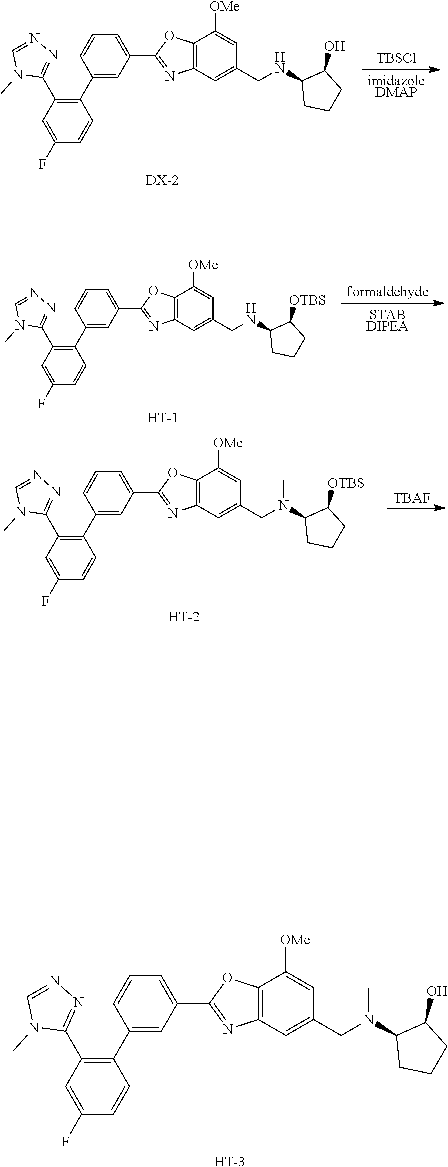

Step 1: (1R,2S)-2-((tert-Butyldimethylsilyl)oxy)-N-((2-(4′-fluoro-2′-(4-methyl-4H-1,2,4-triazol-3-yl)-[1,1′-biphenyl]-3-yl)-7-methoxybenzo[d]oxazol-5-yl)methyl)cyclopentan-1-amine (HT-1). To a stirred solution of compound (DX-2) (70 mg, 1 Eq, 0.14 mmol), imidazole (46 mg, 5 Eq, 0.68 mmol) and DMAP (3.3 mg, 0.2 Eq, 27 μmol) in dry DMF (1 mL) under a nitrogen atmosphere at rt was added TBDMSCl (82 mg, 4.0 Eq, 0.54 mmol). The reaction mixture was stirred at rt for 4 h. The reaction mixture was diluted with distilled water (10 mL) and transferred into a separating funnel. The layer was extracted with EtOAc (3×10 mL). The combined extracted layers were collected, dried (Na₂SO₄), filtered and concentrated in vacuo. The crude product was purified by chromatography on silica gel (4 g cartridge, 0-10% (0.7 M ammonia/MeOH)/DCM) to afford the sub-title compound (HT-1) (70 mg, 0.11 mmol, 80%, 98% Purity) as a sticky colourless gum. m/z 628.3 (M+H)⁺ (ES+). ¹H NMR (400 MHz, MeOD) δ 8.40 (s, 1H), 8.21 (dt, J=8.0, 1.3 Hz, 1H), 8.11 (t, J=1.7 Hz, 1H), 7.81 (dd, J=8.7, 5.4 Hz, 1H), 7.60-7.54 (m, 2H), 7.48 (dd, J=8.8, 2.8 Hz, 1H), 7.40 (ddd, J=7.8, 1.9, 1.1 Hz, 1H), 7.31 (d, J=1.3 Hz, 1H), 7.07 (d, J=1.4 Hz, 1H), 4.34 (q, J=4.1 Hz, 1H), 4.10 (s, 3H), 3.91 (s, 2H), 3.22 (s, 3H), 3.02 (td, J=7.6, 4.1 Hz, 1H), 1.97-1.91 (m, 1H), 1.87-1.74 (m, 3H), 1.66-1.57 (m, 2H), 0.94 (s, 9H), 0.16 (d, J=0.8 Hz, 6H). One exchangeable proton (NH) not observed.

Step 2: (1R,2S)-2-((tert-Butyldimethylsilyl)oxy)-N-((2-(4′-fluoro-2′-(4-methyl-4H-1,2,4-triazol-3-yl)-[1,1′-biphenyl]-3-yl)-7-methoxybenzo[d]oxazol-5-yl)methyl)-N-methylcyclopentan-1-amine (HT-2). A solution of the product from step 1 above (HT-1) (70 mg, 96% Wt, 1 Eq, 0.11 mmol), formaldehyde solution in H₂O (35 mg, 32 μL, 37% Wt, 4 Eq, 0.43 mmol) and DIPEA (83 mg, 0.11 mL, 6 Eq, 0.64 mmol) in CHCl₃ (1 mL) was stirred at 45° C. for 12 h. NaBH(OAc)₃ (0.23 g, 10 Eq, 1.1 mmol) was added and the reaction mixture was stirred at 45° C. for 4 h. The reaction mixture was diluted with DCM (2 mL), washed with sat. NaHCO₃ (2 mL) and brine (2 mL), dried (Na₂SO₄), filtered and concentrated in vacuo. The crude product was purified by chromatography on silica gel (4 g cartridge, 0-10% MeOH/DCM) to afford the sub-title compound (HT-2) (60 mg, 93 μmol, 86%, 99% Purity) as a white solid. ¹H NMR (400 MHz, MeOD) δ 8.40 (s, 1H), 8.21 (d, J=8.1 Hz, 1H), 8.10 (d, J=1.8 Hz, 1H), 7.81 (dd, J=8.6, 5.5 Hz, 1H), 7.57 (td, J=8.3, 3.0 Hz, 2H), 7.48 (dd, J=8.7, 2.8 Hz, 1H), 7.40 (d, J=8.1 Hz, 1H), 7.30 (d, J=1.1 Hz, 1H), 7.13 (d, J=1.3 Hz, 1H), 4.43 (s, 1H), 4.08 (s, 3H), 3.90 (d, J=13.1 Hz, 1H), 3.73 (d, J=12.8 Hz, 1H), 3.22 (s, 3H), 2.60 (s, 1H), 2.27 (s, 3H), 1.92 (d, J=8.1 Hz, 2H), 1.84-1.62 (m, 4H), 0.99 (s, 8H), 0.17 (s, 6H).

Step 3: (1S,2R)-2-(((2-(4′-Fluoro-2′-(4-methyl-4H-1,2,4-triazol-3-yl)-[1,1′-biphenyl]-3-yl)-7-methoxybenzo[d]oxazol-5-yl)methyl)(methyl)amino)cyclopentan-1-ol (HT-3). To a stirred solution of the product from step 2 above (HT-2) (60 mg, 1 Eq, 93 μmol) in dry THF (1.5 mL) under a nitrogen atmosphere at 25° C. was added TBAF in THF (0.11 g, 0.42 mL, 1 molar, 4.5 Eq, 0.42 mmol). The reaction mixture was stirred for 12 h. The reaction mixture was concentrated in vacuo. The reaction mixture was diluted with distilled water (5 mL) and transferred into a separating funnel. The layer was extracted with DCM (3×10 mL). The combined extracted layers were collected, dried (Na₂SO₄), filtered and concentrated in vacuo. The crude product was purified by chromatography on silica gel (4 g cartridge, 0-10% (0.7 M ammonia/MeOH)/DCM) to afford the title compound (HT-3) (35 mg, 65 μmol, 70%, 98% Purity) as a clear, colourless solid. m/z 528.1 (M+H)⁺ (ES+). ¹H NMR (400 MHz, MeOD) δ=8.40 (s, 1H), 8.20 (dt, J=7.9, 1.4 Hz, 1H), 8.09 (t, J=1.7 Hz, 1H), 7.81 (dd, J=8.7, 5.4 Hz, 1H), 7.57 (td, J=8.0, 2.3 Hz, 2H), 7.48 (dd, J=8.8, 2.7 Hz, 1H), 7.41 (ddd, J=7.8, 2.0, 1.1 Hz, 1H), 7.32 (d, J=1.3 Hz, 1H), 7.13 (d, J=1.3 Hz, 1H), 4.29 (d, J=4.0 Hz, 1H), 4.10 (s, 3H), 3.85 (d, J=13.3 Hz, 1H), 3.70 (d, J=13.0 Hz, 1H), 3.22 (s, 3H), 2.62 (s, 1H), 2.26 (s, 3H), 2.02-1.91 (m, 2H), 1.91-1.78 (m, 3H), 1.76-1.66 (m, 1H). Exchangeable OH proton not observed.

### Example 338: N-(2-Cyclobutylethyl)-2-(4′-fluoro-2′-(4-methyl-4H-1,2,4-triazol-3-yl)-[1,1′-biphenyl]-3-yl)-7-(trifluoromethyl)benzo[d]oxazol-5-amine (HU-1)

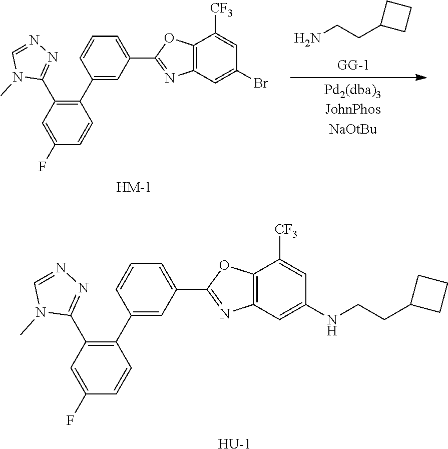

A stirred mixture of intermediate (HM-1) (42 mg, 1 Eq, 81 μmol), 2-cyclobutylethan-1-amine (GG-1) (24 mg, 3.0 Eq, 0.24 mmol), and sodium tert-butoxide (23 mg, 3 Eq, 0.24 mmol) and JohnPhos (4.8 mg, 0.2 eq, 0.016 mmol) in toluene (3 mL) was placed under three vacuum/N₂ cycles at 40° C. before Pd₂(dba)₃ (11 mg, 0.15 Eq, 12 μmol) was added. The mixture was degassed by another three vacuum/N₂ cycles before being warmed to 100° C. and stirring was continued for 3 h. The reaction mixture was concentrated in vacuo. The reaction mixture was diluted with distilled water (5 mL) and transferred into a separating funnel. The layer was extracted with DCM (3×10 mL). The combined extracted layers were collected, dried (Na₂SO₄), filtered and concentrated in vacuo. The crude product was purified by chromatography on silica gel (4 g cartridge, 0-10% MeOH/DCM) to afford the title compound (HU-1) (10 mg, 18 μmol, 23%, 99% Purity) as a pale-yellow solid. m/z 536.5 (M+H)⁺ (ES+). ¹H NMR (400 MHz, MeOD) δ 8.41 (s, 1H), 8.17 (d, J=7.9 Hz, 1H), 8.03 (s, 1H), 7.80 (dd, J=8.6, 5.5 Hz, 1H), 7.60-7.54 (m, 2H), 7.49 (dd, J=8.8, 2.7 Hz, 1H), 7.42 (d, J=7.8 Hz, 1H), 7.04 (s, 1H), 6.98 (s, 1H), 3.26 (s, 3H), 3.12-3.07 (m, 2H), 2.57-2.45 (m, 1H), 2.17 (d, J=8.2 Hz, 2H), 2.02-1.87 (m, 2H), 1.83-1.72 (m, 4H). Exchangeable NH proton not observed.

### Example 339: (1S,2R)-2-((2-(4′-Fluoro-2′-(4-methyl-4H-1,2,4-triazol-3-yl)-[1,1′-biphenyl]-3-yl)-7-(trifluoromethyl)benzo[d]oxazol-5-yl)amino)cyclopentan-1-ol (HV-3)

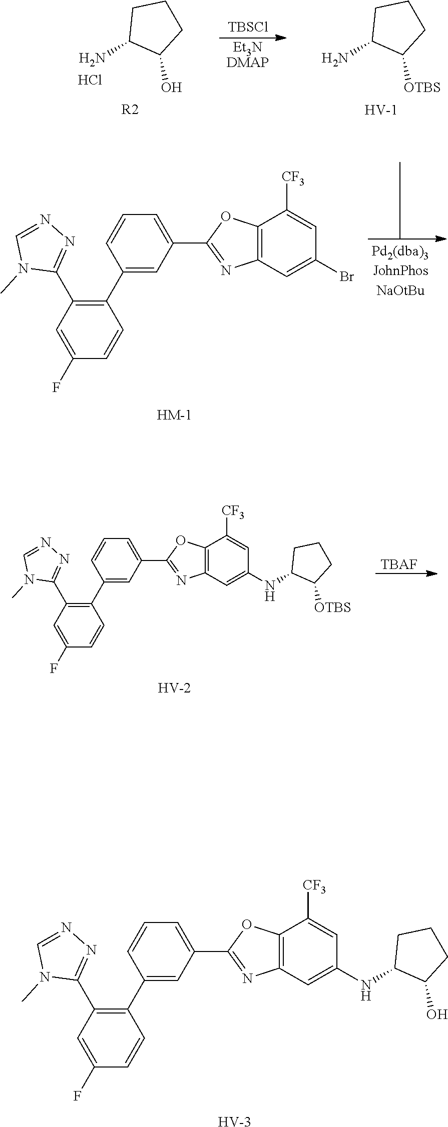

Step 1: (1R,2S)-2-((tert-Butyldimethylsilyl)oxy)cyclopentan-1-amine (HV-1). To a stirred solution of (1S,2R)-2-aminocyclopentan-1-ol, HCl (R-2) (90 mg, 1 Eq, 0.65 mmol), Et₃N (0.32 g, 0.45 mL, 4.9 Eq, 3.2 mmol) and DMAP (8.0 mg, 0.1 Eq, 65 μmol) in dry DMF (1 mL) under a nitrogen atmosphere at rt was added TBDMSCl (0.15 g, 1.55 Eq, 1.0 mmol). The reaction mixture was stirred at rt for 12 h. The reaction mixture was diluted with distilled water (10 mL) and transferred into a separating funnel. The layer was extracted with EtOAc (3×10 mL). The combined extracted layers were collected, dried (Na₂SO₄), filtered and concentrated in vacuo. The crude product was purified by chromatography on silica gel (4 g cartridge, 0-10% (0.7 M ammonia/MeOH)/DCM) to afford the sub-title compound (HV-1) (160 mg, 0.56 mmol, 85%, 75% Purity) as a clear, white solid. m/z 216.2 (M+H)⁺ (ES+). ¹H NMR (400 MHz, DMSO) δ 4.22 (q, J=4.9 Hz, 1H), 3.31 (q, J=4.9 Hz, 1H), 2.01-1.44 (m, 6H), 0.88 (s, 9H), 0.09 (d, J=7.1 Hz, 6H). Exchangeable protons not observed.

Step 2: N-((1R,2S)-2-((tert-Butyldimethylsilyl)oxy)cyclopentyl)-2-(4′-fluoro-2′-(4-methyl-4H-1,2,4-triazol-3-yl)-[1,1′-biphenyl]-3-yl)-7-(trifluoromethyl)benzo[d]oxazol-5-amine (HV-2). A stirred mixture of intermediate (HM-1) (50 mg, 1 Eq, 97 μmol), the product from step 1 above (HV-1) (56 mg, 2 Eq, 0.19 mmol), and NaOtBu (33 mg, 3.5 Eq, 0.34 mmol) and JohnPhos (4.6 mg, 0.2 eq, 0.015 mmol) in toluene (2 mL) was placed under three vacuum/N₂ cycles at 40° C. before Pd₂(dba)₃ (13 mg, 0.15 Eq, 14 μmol) was added. The mixture was degassed by another three vacuum/N₂ cycles before being warmed to 100° C. and stirring was continued for 3 h. The reaction mixture was concentrated in vacuo. The crude product was purified by chromatography on silica gel (4 g cartridge, 0-10% MeOH/DCM) to afford the sub-title compound (HV-2) (35 mg, 27 μmol, 28%, 50% Purity) as a sticky orange gum, which was taken forward without further purification or analysis.

Step 3: (1S,2R)-2-((2-(4′-fluoro-2′-(4-methyl-4H-1,2,4-triazol-3-yl)-[1,1′-biphenyl]-3-yl)-7-(trifluoromethyl)benzo[d]oxazol-5-yl)amino)cyclopentan-1-ol (HV-3). To a stirred solution of the product from step 2 above (HV-2) (35 mg, 50% Wt, 1 Eq, 27 μmol) in dry THF (1.5 mL) under a nitrogen atmosphere at 25° C. was added TBAF in THF (32 mg, 0.12 mL, 1 molar, 4.5 Eq, 0.12 mmol). The reaction mixture was stirred for 12 h. The reaction mixture was concentrated in vacuo. The reaction mixture was diluted with distilled water (5 mL) and transferred into a separating funnel. The layer was extracted with DCM (3×10 mL). The combined extracted layers were collected, dried (Na₂SO₄), filtered and concentrated in vacuo. The crude product was purified by chromatography on silica gel (4 g cartridge, 0-10% (0.7 M ammonia/MeOH)/DCM) to afford the title compound (HV-3) (4 mg, 7 μmol, 30%, 98% Purity) as a pale, yellow solid. m/z 538.1 (M+H)⁺ (ES+). ¹H NMR (400 MHz, MeOD) δ=8.41 (s, 1H), 8.17 (dt, J=8.0, 1.3 Hz, 1H), 8.03 (t, J=1.7 Hz, 1H), 7.80 (dd, J=8.6, 5.5 Hz, 1H), 7.63-7.54 (m, 2H), 7.49 (dd, J=8.8, 2.7 Hz, 1H), 7.42 (ddd, J=7.8, 1.9, 1.1 Hz, 1H), 7.16 (d, J=2.2 Hz, 1H), 7.12-7.06 (m, 1H), 4.38-4.26 (m, 1H), 3.72 (td, J=8.0, 4.4 Hz, 1H), 3.26 (s, 3H), 2.18-2.07 (m, 1H), 2.00 (ddt, J=17.4, 7.9, 4.4 Hz, 1H), 1.95-1.84 (m, 1H), 1.85-1.75 (m, 1H), 1.76-1.61 (m, 2H). Two exchangeable protons (NH & OH) not observed.

### Example 340: 1-(2-(4′-Fluoro-2′-(4-methyl-4H-1,2,4-triazol-3-yl)-[1,1′-biphenyl]-3-yl)-7-(trifluoromethyl)benzo[d]oxazol-5-yl)-N-((1-fluorocyclobutyl)methyl)methanamine (HW-2)

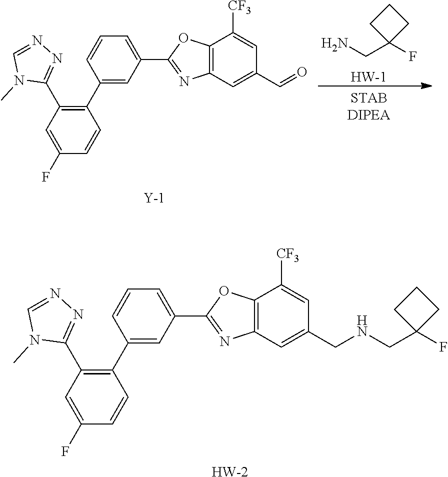

NaBH(OAc)₃ (36 mg, 3.0 Eq, 0.17 mmol) was added to a stirred mixture of intermediate (Y-1) (35 mg, 76% Wt, 1 Eq, 57 μmol), (1-fluorocyclobutyl)methanamine, HCl (HW-1) (16 mg, 2.0 Eq, 0.11 mmol) and DIPEA (22 mg, 30 μL, 3.0 Eq, 0.17 mmol) in CHCl₃ (3 mL). The reaction mixture was stirred at 40° C. overnight. More NaBH(OAc)₃ (36 mg, 3.0 Eq, 0.17 mmol) was added to the reaction mixture and stirring continued at 40° C. overnight. The reaction mixture was diluted with DCM (4 mL) and washed with sat. aq. NaHCO₃ (10 mL). The organic phase was separated and evaporated to afford the crude product. The crude product was purified by preparative HPLC (Waters, Basic (0.3% ammonia), Basic, Waters X-Bridge BEH C18 ODB, 5 μm, 45×100 mm column 15-100% MeCN in water) to afford the title compound (HW-2) (15 mg, 27 μmol, 47%, 99% Purity) as a white solid. m/z 554.3 (M+H)⁺ (ES+). ¹H NMR (400 MHz, DMSO) δ 8.42 (s, 1H), 8.13 (dt, J=7.9, 1.4 Hz, 1H), 8.08 (s, 1H), 7.91 (t, J=1.8 Hz, 1H), 7.83-7.74 (m, 2H), 7.65-7.51 (m, 3H), 7.38 (dt, J=7.8, 1.5 Hz, 1H), 3.95 (s, 2H), 3.20 (s, 3H), 2.77 (s, 1H), 2.71 (s, 1H), 2.22-2.10 (m, 4H), 1.73 (d, J=9.4 Hz, 1H), 1.46-1.35 (m, 1H). Single exchangeable proton not observed.

### Example 341: (1S,2R)-2-(((6,7-Difluoro-2-(4′-fluoro-2′-(4-methyl-4H-1,2,4-triazol-3-yl)-[1,1′-biphenyl]-3-yl)benzo[d]oxazol-5-yl)methyl)amino)cyclopentan-1-ol (HX-8)

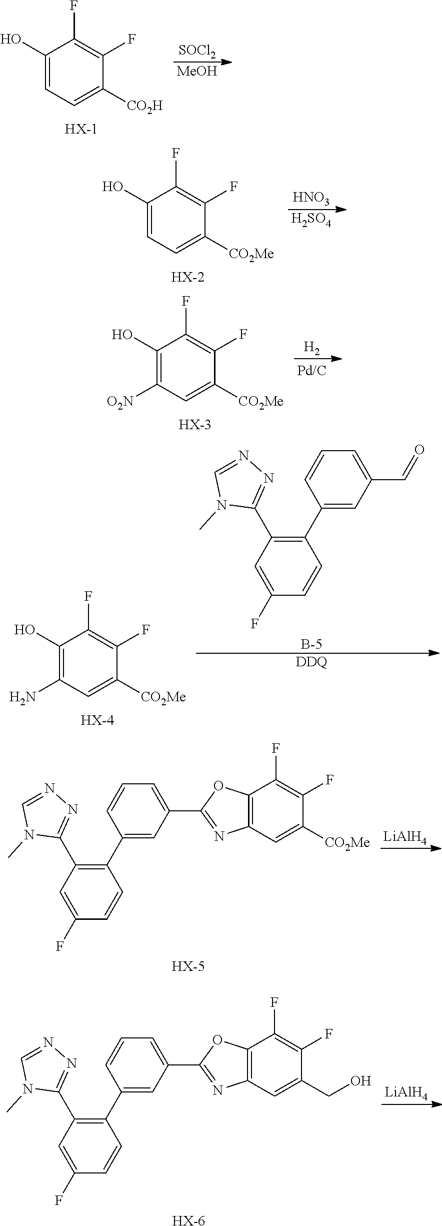

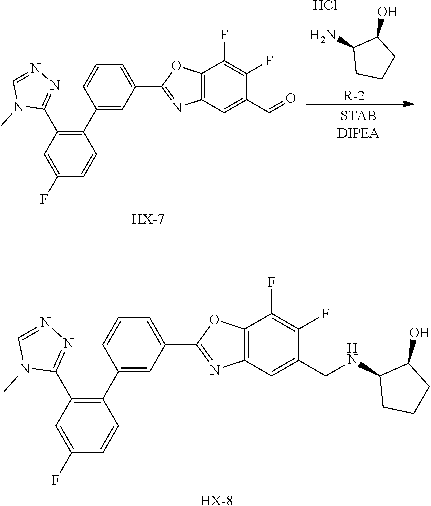

Step 1: Methyl 2,3-difluoro-4-hydroxybenzoate (HX-2). To a stirred solution of 2,3-difluoro-4-hydroxybenzoicacid (HX-1) (992.4 mg, 1 Eq, 5.700 mmol) in dry MeOH (50 mL) under a nitrogen atmosphere at rt was added SOCl₂ (2.8 g, 1.7 mL, 4.1 Eq, 23 mmol) dropwise. The reaction mixture was heated at 64° C. overnight. The reaction was cooled to rt. The volatiles were removed in vacuo to afford the sub-title compound (HX-2) (1.072 g, 5.6 mmol, 99%, 99% Purity) as a beige solid. m/z 186.7 (M−H)-(ES−). ¹H NMR (400 MHz, DMSO) δ 11.38 (s, 1H), 7.57 (ddd, J=9.0, 8.0, 2.2 Hz, 1H), 6.87 (ddd, J=9.3, 7.8, 1.9 Hz, 1H), 3.81 (s, 3H).

Step 2: Methyl 2,3-difluoro-4-hydroxy-5-nitrobenzoate (HX-3). To a mixture of the product from step 1 above (HX-2) (1.072 g, 99% Wt, 1 Eq, 5.641 mmol) suspended in conc. sulfuric acid (18 g, 10 mL, 33 Eq, 0.19 mol) was added nitric acid (1.13 g, 750 μL, 3.19 Eq, 18.0 mmol) dropwise at 0° C. The resulting suspension was stirred at this temperature for 10 min. The mixture was poured into ice water (10 mL). A precipitation was observed, the product was filtered and dried to afford the sub-title compound (HX-3) (659.9 mg, 2.8 mmol, 49%, 98% Purity) as an orange solid. m/z 231.7 (M−H)⁻ (ES−). ¹H NMR (400 MHz, DMSO) δ 8.27 (dd, J=7.5, 2.2 Hz, 1H), 3.86 (s, 3H). 1× exchangeable OH proton not observed.

Step 3: Methyl 5-amino-2,3-difluoro-4-hydroxybenzoate (HX-4). To a solution of the product from step 2 above (HX-3) (659.9 mg, 98% Wt, 1 Eq, 2.774 mmol) in MeOH (20 mL) was added Pd/C 87 L (296.4 mg, 5% Wt, 0.05020 Eq, 139.3 μmol). The reaction mixture was stirred at rt with 5 atm of H₂ overnight. The mixture was filtered and the volatiles were removed in vacuo to afford the sub-title compound (HX-4) (532.1 mg, 2.5 mmol, 89%, 94% Purity) as a pale brown solid. m/z 204.3 (M+H)⁺ (ES+). ¹H NMR (400 MHz, DMSO) δ 6.98 (s, 1H), 3.79 (s, 3H). 3× exchangeable protons not observed.

Step 4: Methyl 6,7-difluoro-2-(4′-fluoro-2′-(4-methyl-4H-1,2,4-triazol-3-yl)-[1,1′-biphenyl]-3-yl)benzo[d]oxazole-5-carboxylate (HX-5). To a solution of intermediate (B-5) (429.8 mg, 73% Wt, 1 Eq, 1.115 mmol) in MeOH (10 mL) was added the product from step 3 above (HX-4) (251.5 mg, 94% Wt, 1.043 Eq, 1.164 mmol). The resulting mixture was stirred at 45° C. overnight. The reaction mixture was cooled to rt and the volatiles were removed in vacuo. The residue was redissolved in DCM (10 mL). DDQ (395.2 mg, 98% Wt, 1.53 Eq, 1.71 mmol) was added and the resulting mixture was stirred at rt for 1 h. The crude product was purified by chromatography on silica gel (24 g cartridge, 0-10% MeOH/DCM) to afford an inseparable mixture of the sub-title compound (HX-5) (75.3 mg, 66 μmol, 6.0%, 41% Purity) and unreacted (B-5) (75.3 mg, 0.13 mmol, 12%, 49% Purity) as a brown solid and additional unreacted (B-5) (282.6 mg, 0.45 mmol, 41%, 45% Purity) as a brown solid. m/z 465.5 (M+H)⁺ (ES+).

Step 5: (6,7-Difluoro-2-(4′-fluoro-2′-(4-methyl-4H-1,2,4-triazol-3-yl)-[1,1′-biphenyl]-3-yl)benzo[d]oxazol-5-yl)methanol (HX-6). To a solution of the product from step 4 above (HX-5) (75.3 mg, 41% Wt, 1 Eq, 66.5 μmol) (containing 49 wt % B-5) in THF (3.0 mL) was added LiAlH₄ in THF (6.07 mg, 160 μL, 1 molar, 2.41 Eq, 160 μmol) dropwise at 0° C. This mixture was stirred at 0° C. for 1 h. The mixture was quenched with H₂O (0.5 mL) and NaOH (2 M, 0.5 mL) at 0° C. Na₂SO₄ was added. The mixture was warmed up to rt, stirred for 15 min, filtered and the solvent was removed in vacuo. The crude product was purified by chromatography on silica gel (12 g cartridge, 0-10% MeOH/DCM) to afford the sub-title compound (HX-6) (15.6 mg, 33 μmol, 49%, 91% Purity) as a pale-yellow solid. m/z 437.1 (M+H)⁺ (ES+). ¹H NMR (400 MHz, MeOD) δ 8.38 (s, 1H), 8.20 (dt, J=7.7, 1.3 Hz, 1H), 8.08 (t, J=1.8 Hz, 1H), 7.78 (dd, J=8.6, 5.5 Hz, 1H), 7.65-7.61 (m, 1H), 7.58-7.51 (m, 2H), 7.46 (dd, J=8.7, 2.8 Hz, 1H), 7.41 (ddd, J=7.8, 1.9, 1.1 Hz, 1H), 4.79 (dd, J=1.7, 0.8 Hz, 2H), 3.20 (s, 3H). 1× exchangeable OH proton not observed.

Step 6: 6,7-Difluoro-2-(4′-fluoro-2′-(4-methyl-4H-1,2,4-triazol-3-yl)-[1,1′-biphenyl]-3-yl)benzo[d]oxazole-5-carbaldehyde (HX-7). To a stirred solution of the product from step 5 above (HX-6) (15.6 mg, 91% Wt, 1 Eq, 32.5 μmol) in dry DCM (2.0 mL) at rt was added Dess-Martin periodinane (29.2 mg, 2.12 Eq, 68.8 μmol). The reaction mixture was stirred at rt for 1 h. The reaction mixture was diluted with DCM (5 mL), washed with sat. NaHCO₃ (5 mL) and brine (5 mL). The organic layer was dried (Na₂SO₄), filtered and concentrated in vacuo. The crude product was purified by chromatography on silica gel (4 g cartridge, 0-10% MeOH/DCM) to afford the sub-title compound (HX-7) (16.6 mg, 31 μmol, 95%, 81% Purity) as a pale-yellow solid. m/z 435.0 (M+H)⁺ (ES+).

Step 7: (1S,2R)-2-(((6,7-Difluoro-2-(4′-fluoro-2′-(4-methyl-4H-1,2,4-triazol-3-yl)-[1,1′-biphenyl]-3-yl)benzo[d]oxazol-5-yl)methyl)amino)cyclopentan-1-ol (HX-8). A solution of the product from step 6 above (HX-7) (16.6 mg, 81% Wt, 1 Eq, 31.0 μmol), (1S,2R)-2-aminocyclopentan-1-ol, HCl (R-2) (9.5 mg, 2.2 Eq, 69 μmol) and DIPEA (22 mg, 30 μL, 5.6 Eq, 0.17 mmol) in CHCl₃ (2.0 mL) was stirred 1 h at rt. NaBH(OAc)₃ (20.3 mg, 97% Wt, 3.00 Eq, 92.9 μmol) was added and the reaction mixture was stirred at rt overnight. NaBH(OAc)₃ (22.8 mg, 97% Wt, 3.37 Eq, 104 μmol) was added and the reaction mixture was stirred at rt for 2 h. Further NaBH(OAc)₃ (21.8 mg, 97% Wt, 3.22 Eq, 99.8 μmol) was added and the resulting mixture was stirred at rt for 1 h. The reaction mixture was diluted with EtOAc (5 mL), washed with sat. NaHCO₃ (5 mL) and brine (5 mL), dried (Na₂SO₄), filtered and concentrated in vacuo. The crude product was purified by chromatography on silica gel (4 g cartridge, 0-10% MeOH/DCM) to afford the title compound (HX-8) (4.6 mg, 8.8 μmol, 28%, 99% Purity) as an off-white solid. m/z 520.4 (M+H)⁺ (ES+). ¹H NMR (400 MHz, MeOD) δ 8.38 (s, 1H), 8.19 (dt, J=7.9, 1.4 Hz, 1H), 8.07 (t, J=1.8 Hz, 1H), 7.77 (dd, J=8.6, 5.5 Hz, 1H), 7.66-7.62 (m, 1H), 7.58-7.51 (m, 2H), 7.46 (dd, J=8.8, 2.8 Hz, 1H), 7.41 (dt, J=7.8, 1.4 Hz, 1H), 4.13 (td, J=4.4, 2.4 Hz, 1H), 4.05-4.00 (m, 1H), 4.00-3.95 (m, 1H), 3.19 (s, 3H), 2.99 (td, J=8.0, 4.2 Hz, 1H), 1.95-1.88 (m, 1H), 1.88-1.78 (m, 2H), 1.77-1.67 (m, 1H), 1.63-1.48 (m, 2H). 2× exchangeable protons not observed.

### Example 342: (1S,2R)-2-(((2-(4′-Fluoro-2′-(4-methyl-4H-1,2,4-triazol-3-yl)-[1,1′-biphenyl]-3-yl)-7-methylbenzo[d]oxazol-5-yl)methyl)(methyl)amino)cyclopentan-1-ol (HY-3)

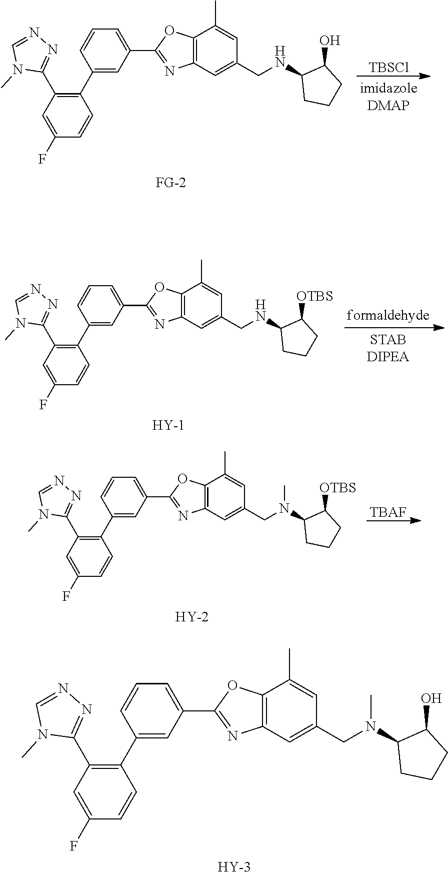

Step 1: (1R,2S)-2-((tert-Butyldimethylsilyl)oxy)-N-((2-(4′-fluoro-2′-(4-methyl-4H-1,2,4-triazol-3-yl)-[1,1′-biphenyl]-3-yl)-7-methylbenzo[d]oxazol-5-yl)methyl)cyclopentan-1-amine (HY-1). To a stirred solution of compound (FG-2) (64 mg, 95% Wt, 1 Eq, 0.12 mmol), imidazole (42 mg, 5 Eq, 0.61 mmol) and DMAP (3.0 mg, 0.2 Eq, 24 μmol) in dry DMF (1 mL) under a nitrogen atmosphere at rt was added TBDMSCl (74 mg, 4 Eq, 0.49 mmol). The reaction mixture was stirred at rt for 4 h. Additional TBDMSCl (74 mg, 4 Eq, 0.49 mmol) in DMF (0.5 mL) was added and stirred at rt for 16 h. The reaction mixture was diluted with distilled water (10 mL) and transferred into a separating funnel. The layer was extracted with EtOAc (3×10 mL). The combined extracted layers were collected, dried (Na₂SO₄), filtered and concentrated in vacuo. The crude product was purified by chromatography on silica gel (4 g cartridge, 0-10% MeOH/DCM) to afford the sub-title compound (HY-1) (44.9 mg, 70 μmol, 57%, 95% Purity) as a white solid. m/z 612.3 (M+H)⁺ (ES+). ¹H NMR (400 MHz, MeOD) δ 8.41 (s, 1H), 8.26-8.21 (m, 1H), 8.10-8.08 (m, 1H), 7.82 (dd, J=8.6, 5.5 Hz, 1H), 7.62-7.54 (m, 3H), 7.49 (dd, J=8.7, 2.7 Hz, 1H), 7.47-7.42 (m, 1H), 7.27 (s, 1H), 4.33 (d, J=4.0 Hz, 1H), 3.91 (s, 2H), 3.21 (s, 3H), 3.07-2.98 (m, 1H), 2.63 (s, 3H), 2.01-1.70 (m, 4H), 1.67-1.53 (m, 2H), 0.94 (s, 9H), 0.15 (s, 6H). One exchangeable proton not observed.

Step 2: (1R,2S)-2-((tert-Butyldimethylsilyl)oxy)-N-((2-(4′-fluoro-2′-(4-methyl-4H-1,2,4-triazol-3-yl)-[1,1′-biphenyl]-3-yl)-7-methylbenzo[d]oxazol-5-yl)methyl)-N-methylcyclopentan-1-amine (HY-2). A solution of the product from step 1 above (HY-1) (44.9 mg, 95% Wt, 1 Eq, 69.7 μmol), formaldehyde solution in H₂O (17.0 mg, 15.6 μL, 37% Wt, 3 Eq, 209 μmol) and DIPEA (45.1 mg, 60.7 μL, 5 Eq, 349 μmol) in CHCl₃ (1 mL) was stirred at rt for 12 h. NaBH(OAc)₃ (73.9 mg, 5 Eq, 349 μmol) was added and the reaction mixture was stirred at rt overnight. The reaction mixture was diluted with DCM (2 mL), washed with sat. NaHCO₃ (2 mL) and brine (2 mL), dried (Na₂SO₄), filtered and concentrated in vacuo. The crude product was purified by chromatography on silica gel (4 g cartridge, 0-10% MeOH/DCM) to afford the sub-title compound (HY-2) (34.1 mg, 52 μmol, 74%, 95% Purity) as a white solid. m/z 626.3 (M+H)⁺ (ES+).

Step 3: (1S,2R)-2-(((2-(4′-Fluoro-2′-(4-methyl-4H-1,2,4-triazol-3-yl)-[1,1′-biphenyl]-3-yl)-7-methylbenzo[d]oxazol-5-yl)methyl)(methyl)amino)cyclopentan-1-ol (HY-3). To a stirred solution of the product from step 2 above (HY-2) (34.1 mg, 95% Wt, 1 Eq, 51.8 μmol) in dry THF (1.5 mL) under a nitrogen atmosphere at 25° C. was added TBAF in THF (207 mg, 0.233 mL, 1 molar, 4.50 Eq, 233 μmol). The reaction mixture was stirred for 12 hours. The reaction mixture was concentrated in vacuo. The reaction mixture was diluted with distilled water (5 mL) and transferred into a separating funnel. The layer was extracted with DCM (3×10 mL). The combined extracted layers were collected, dried (Na₂SO₄), filtered and concentrated in vacuo. The crude product was purified by chromatography on silica gel (4 g cartridge, 0-10% MeOH/DCM) to afford the title compound (HY-3) (15.27 mg, 29 μmol, 57%, 98% Purity) as a flocculent white solid. m/z 512.2 (M+H)⁺ (ES+). ¹H NMR (400 MHz, DMSO) δ 8.43 (s, 1H), 8.13 (dt, J=7.9, 1.3 Hz, 1H), 7.91 (t, J=1.8 Hz, 1H), 7.78 (dd, J=8.7, 5.7 Hz, 1H), 7.64-7.51 (m, 4H), 7.34 (dt, J=7.9, 1.3 Hz, 1H), 7.26 (s, 1H), 4.11 (s, 1H), 3.83 (d, J=2.6 Hz, 1H), 3.69 (d, J=13.1 Hz, 1H), 3.58 (d, J=13.2 Hz, 1H), 3.15 (s, 3H), 2.55 (s, 3H), 2.48-2.41 (m, 1H), 2.10 (s, 3H), 1.88-1.48 (m, 6H).

### Example 343: N-(2-(Azetidin-1-yl)ethyl)-2-(4′-fluoro-2′-(4-methyl-4H-1,2,4-triazol-3-yl)-[1,1′-biphenyl]-3-yl)-7-(trifluoromethyl)benzo[d]oxazol-5-amine (HZ-2)

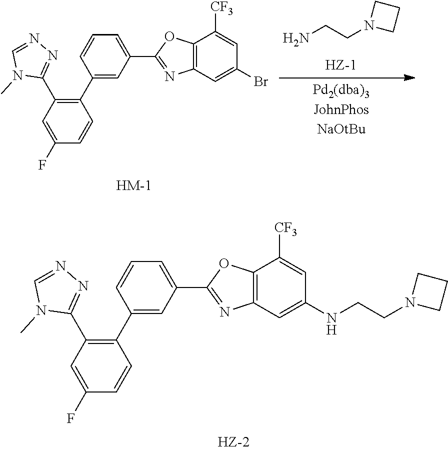

A stirred mixture of intermediate (HM-1) (40 mg, 1 Eq, 77 μmol), 2-(azetidin-1-yl)ethan 1-amine (HZ-1) (19 mg, 2.5 Eq, 0.19 mmol), and NaOtBu (26 mg, 3.5 Eq, 0.27 mmol) and JohnPhos (4.6 mg, 0.2 eq, 0.015 mmol) in toluene (2 mL) was placed under three vacuum/N₂ cycles at 40° C. before Pd₂(dba)₃ (11 mg, 0.15 Eq, 12 μmol) was added. The mixture was degassed by another three vacuum/N₂ cycles before being warmed to 100° C. and stirring was continued for 3 h. The reaction mixture was concentrated in vacuo. The crude product was purified by chromatography on silica gel (4 g cartridge, 0-10% MeOH/DCM) to afford the title compound (HZ-2) (12 mg, 22 μmol, 29%, 99% Purity) as a pale-yellow solid. m/z 537.2 (M+H)⁺ (ES+). ¹H NMR (400 MHz, MeOD) δ=8.41 (s, 1H), 8.17 (dt, J=7.8, 1.3 Hz, 1H), 8.04 (t, J=1.8 Hz, 1H), 7.80 (dd, J=8.7, 5.4 Hz, 1H), 7.61-7.53 (m, 2H), 7.49 (dd, J=8.8, 2.7 Hz, 1H), 7.42 (ddd, J=7.7, 1.9, 1.1 Hz, 1H), 7.12-7.06 (m, 1H), 7.02-6.96 (m, 1H), 3.39 (d, t=7.2 Hz, 4H), 3.26 (s, 3H), 3.21 (t, J=6.6 Hz, 2H), 2.76 (t, J=6.6 Hz, 2H), 2.21-2.15 (m, 2H). Exchangeable NH proton not observed.

### Example 344: 1-((((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)(methyl)amino)methyl)cyclobutan-1-ol (IA-1)

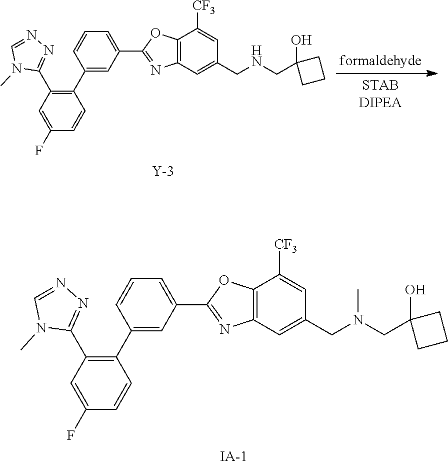

A solution of compound (Y-3) (27.1 mg, 950% Wt, 1 Eq, 46.7 μmol), formaldehyde solution in H₂O (11.4 mg, 10.4 μL, 37% Wt, 3 Eq, 140 μmol) and DIPEA (30.2 mg, 40.7 μL, 5 Eq, 233 μmol) in MeOH (2 mL) was stirred at rt for 12 h. NaBH(OAc)₃ (49.5 mg, 5 Eq, 233 μmol) was added and the reaction mixture was stirred at rt overnight. The reaction mixture was diluted with DCM (2 mL), washed with sat. NaHCO₃ (2 mL) and brine (2 mL), dried (Na₂SO₄), filtered and concentrated in vacuo. The crude product was purified by chromatography on silica gel (4 g cartridge, 0-10% MeOH/DCM) to afford the title compound (IA-1) (7.07 mg, 12 μmol, 25%, 95% Purity) as a white solid. m/z 566.1 (M+H)⁺ (ES+). ¹H NMR (400 MHz, DMSO) δ 8.42 (s, 1H), 8.13 (dt, J=7.9, 1.5 Hz, 1H), 8.07 (s, 1H), 7.90 (t, J=1.8 Hz, 1H), 7.80-7.73 (m, 2H), 7.64-7.53 (m, 3H), 7.40-7.35 (m, 1H), 4.92 (s, 1H), 3.78 (s, 2H), 3.20 (s, 3H), 2.48 (s, 2H), 2.27 (s, 3H), 2.06-1.97 (m, 2H), 1.97-1.84 (m, 2H), 1.65-1.55 (m, 1H), 1.37-1.21 (m, 1H).

### Example 345: (1S,2R)-2-(((2-(4′-Fluoro-2′-(4-methyl-4H-1,2,4-triazol-3-yl)-[1,1′-biphenyl]-3-yl)oxazolo[4,5-b]pyridin-5-yl)methyl)amino)cyclopentan-1-ol (IB-7)

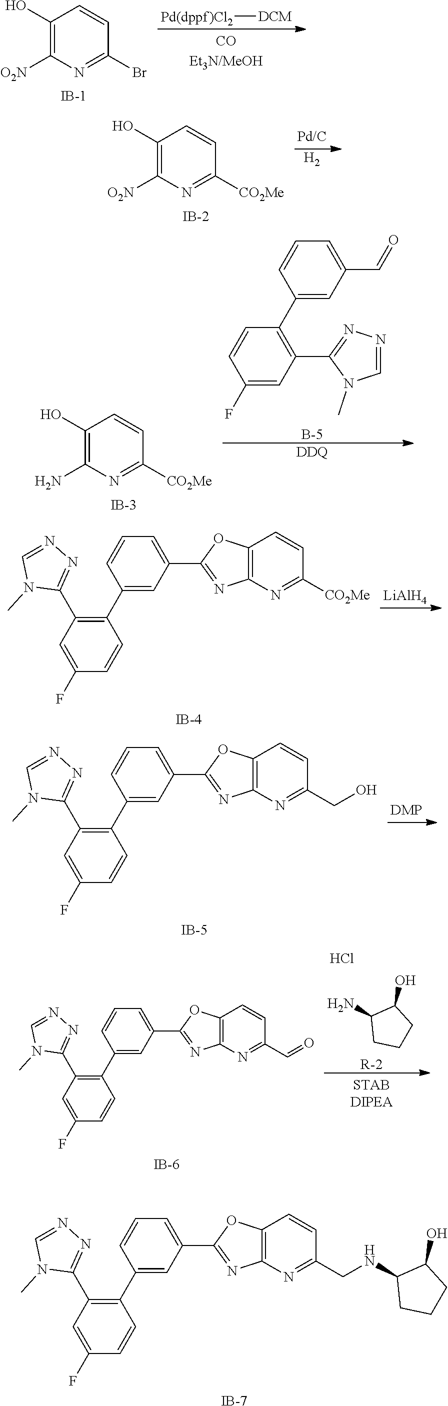

Step 1: Methyl 5-hydroxy-6-nitropicolinate (IB-2). To a solution of 6-bromo-2-nitropyridin-3-ol (IB-1) (1 g, 1 Eq, 5 mmol) and Pd(dppf)Cl₂-DCM (0.4 g, 0.1 Eq, 0.5 mmol) in MeOH (10 mL) and DMF (2 mL) was added Et₃N (2 g, 3 mL, 4 Eq, 0.02 mol). The vessel was sealed, purged with nitrogen three times, purged with carbon monoxide three times and pressurized to 5 bar with carbon monoxide. The reaction mixture was heated to 100° C. and stirred for 4 h. The vessel was cooled to rt and was purged with nitrogen three times. The reaction mixture was then concentrated in vacuo to give a crude brown residue. The reaction mixture was diluted with distilled water (100 mL) and transferred into a separating funnel. The layer was extracted with DCM (1×50 mL). The combined extracted layers were collected and discarded. The water layer was taken and adjusted the pH around 4-5 with 1N HCl. The layer was extracted with EtOAc (3×25 mL). The combined extracted layers were collected, dried (Na₂SO₄), filtered and concentrated in vacuo to afford the sub-title compound (IB-2) (550 mg, 2.4 mmol, 50%, 87% Purity) as a sticky brown solid. m/z 199.2 (M+H)⁺ (ES+). ¹H NMR (400 MHz, DMSO) δ 8.24 (d, J=8.6 Hz, 1H), 7.73 (d, J=8.6 Hz, 1H), 3.86 (s, 3H). Exchangeable OH not observed.

Step 2: Methyl 6-amino-5-hydroxypicolinate (IB-3). To a solution of the product from step 1 above (IB-2) (550 mg, 87% Wt, 1 Eq, 2.42 mmol) in MeOH (5 mL) was added Pd/C (257 mg, 10% Wt, 0.1 Eq, 242 μmol). The reaction mixture was stirred at rt with 25° C. of H₂ for 2 h. After completion of the reaction, the palladium was filtered off and the reaction mixture was concentrated in vacuo to afford the sub-title compound (IB-3) (310 mg, 1.8 mmol, 76%, 99% Purity) as a pale-brown solid. m/z 169.1 (M+H)⁺ (ES+).

Step 3: Methyl 2-(4′-fluoro-2′-(4-methyl-4H-1,2,4-triazol-3-yl)-[1,1′-biphenyl]-3-yl)oxazolo[4,5-b]pyridine-5-carboxylate (IB-4). To a solution of intermediate (B-5) (353 mg, 64% Wt, 1.0 Eq, 803 μmol) in MeOH (5 mL) was added the product from step 2 above (IB-3) (150 mg, 90% Wt, 1.0 Eq, 803 μmol) in a vial. The resulting mixture was stirred at 50° C. for over 12 h. DDQ (182 mg, 1 Eq, 803 μmol) was added and continue the stirring at rt for another 2 h. The reaction mixture was diluted with Sat. NaHCO₃ (10 mL) and transferred into a separating funnel. The layer was extracted with DCM (3×10 mL). The combined extracted layers were collected, dried (Na₂SO₄), filtered and concentrated in vacuo. The crude product was purified by chromatography on silica gel (12 g cartridge, 0-10% MeOH/DCM) to afford the sub-title compound (IB-4) (37 mg, 84 μmol, 11%, 98% Purity) as a light green solid. ¹H NMR (400 MHz, MeOD) δ=8.42 (s, 1H), 8.33 (dt, J=7.9, 1.3 Hz, 1H), 8.31-8.26 (m, 2H), 8.19 (t, J=1.8 Hz, 1H), 7.82 (dd, J=8.7, 5.5 Hz, 1H), 7.66-7.55 (m, 2H), 7.54-7.48 (m, 2H), 4.05 (s, 3H), 3.26 (d, J=3.4 Hz, 3H).

Step 4: (2-(4′-Fluoro-2′-(4-methyl-4H-1,2,4-triazol-3-yl)-[1,1′-biphenyl]-3-yl)oxazolo[4,5-b]pyridin-5-yl)methanol (IB-5). To a solution of the product from step 3 above (IB-4) (35 mg, 1 Eq, 82 μmol) in THF (2 mL) was added LiAlH₄ (3.7 mg, 98 μL, 1 molar, 1.2 Eq, 98 μmol) dropwise at 0° C. This mixture was stirred 1 h at this temperature. The reaction mixture was diluted with distilled water (10 mL) and acidified with 2 mL 1M HCl and transferred into a separating funnel. The layer was extracted with DCM (3×10 mL). The combined extracted layers were collected, dried (Na₂SO₄), filtered and concentrated in vacuo. The crude product was purified by chromatography on silica gel (40 g cartridge, 0-10% (0.7 M ammonia/MeOH)/DCM) to afford the sub-title compound (IB-5) (30 mg, 46 μmol, 57%, 62% Purity) as a thick, colourless solid. m/z 402.1 (M+H)⁺ (ES+).

Step 5: 2-(4′-Fluoro-2′-(4-methyl-4H-1,2,4-triazol-3-yl)-[1,1′-biphenyl]-3-yl)oxazolo[4,5-b]pyridine-5-carbaldehyde (IB-6). To a stirred solution of the product from step 4 above (IB-5) (35 mg, 60% Wt, 1 Eq, 52 μmol) in dry DCM (2 mL) under a nitrogen atmosphere at 25° C. was added DMP (44 mg, 2 Eq, 0.10 mmol). The reaction mixture was stirred for 1 h. The reaction mixture was diluted with DCM (10 mL) and washed with sat. aq. NaHCO₃ (10 mL) and brine. Organics were separated, dried (MgSO₄), filtered and evaporated in vacuo. The crude product was purified by chromatography on silica gel (40 g cartridge, 0-10% (0.7 M ammonia/MeOH)/DCM) to afford the sub-title compound (IB-6) (18 mg, 37 μmol, 72%, 83% Purity) as a pale-yellow solid. m/z 400.2 (M+H)⁺ (ES+).

Step 6: (1S,2R)-2-(((2-(4′-Fluoro-2′-(4-methyl-4H-1,2,4-triazol-3-yl)-[1,1′-biphenyl]-3-yl)oxazolo[4,5-b]pyridin-5-yl)methyl)amino)cyclopentan-1-ol (IB-7). A solution of the product from step 5 above (IB-6) (18 mg, 80% Wt, 1 Eq, 36 μmol), (1S,2R)-2-aminocyclopentan-1-ol, HCl (R-2) (9.9 mg, 2 Eq, 72 μmol) and DIPEA (23 mg, 31 μL, 5 Eq, 0.18 mmol) in CHCl₃ (10 mL) was stirred for at 40° C. over the weekend. NaBH(OAc)₃ (38 mg, 5 Eq, 0.18 mmol) was added and the reaction mixture was stirred at 40° C. for 2 h. The reaction mixture was diluted with saturated NaHCO₃ solution (5 mL) and transferred into a separating funnel. The layer was extracted with DCM (3×5 mL). The combined extracted layers were collected, dried (Na₂SO₄), filtered and concentrated in vacuo. The crude product was purified by chromatography on silica gel (4 g cartridge, 0-10% (0.7 M ammonia/MeOH)/DCM) to afford the title compound (IB-7) (6 mg, 0.01 mmol, 30%, 98% Purity) as a clear colourless solid. m/z 485.5 (M+H)⁺ (ES+). ¹H NMR (400 MHz, MeOD) δ 8.41 (s, 1H), 8.33-8.26 (m, 1H), 8.19-8.11 (m, 2H), 7.81 (dd, J=8.6, 5.5 Hz, 1H), 7.67-7.48 (m, 5H), 4.38-4.27 (m, 3H), 3.30-3.26 (m, 1H), 3.25 (s, 3H), 2.06 (d, J=8.6 Hz, 1H), 1.96-1.85 (m, 2H), 1.79 (d, J=12.1 Hz, 2H), 1.66 (s, 1H). Exchangeable NH & OH proton not observed.

### Example 346: 1-(2-(4′-Fluoro-2′-(4-methyl-4H-1,2,4-triazol-3-yl)-[1,1′-biphenyl]-3-yl)-7-methylbenzo[d]oxazol-5-yl)-N-((1-fluorocyclobutyl)methyl)methanamine (IC-1)

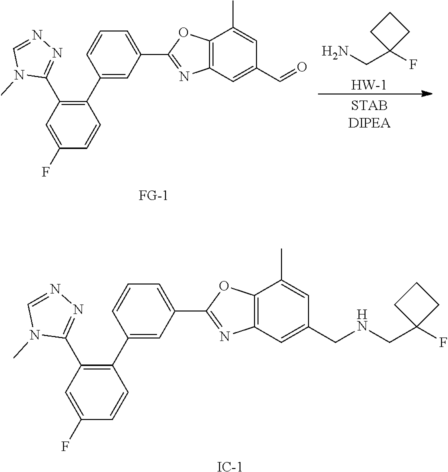

A solution of intermediate (FG-1) (30 mg, 80% Wt, 1 Eq, 58 μmol), (1-fluorocyclobutyl)methanamine (HW-1) (16 mg, 2.0 Eq, 0.12 mmol) and DIPEA (38 mg, 51 μL, 5 Eq, 0.29 mmol) in CHCl₃ (10 mL) was stirred for at 40° C. for 12h. NaBH(OAc)₃ (62 mg, 5 Eq, 0.29 mmol) was added and the reaction mixture was stirred at 40° C. for 5 h. The reaction mixture was diluted with saturated NaHCO₃ solution (5 mL) and transferred into a separating funnel. The layer was extracted with DCM (3×5 mL). The combined extracted layers were collected, dried (Na₂SO₄), filtered and concentrated in vacuo. The crude product was purified by chromatography on silica gel (4 g cartridge, 0-10% (0.7 M ammonia/MeOH)/DCM) to afford the title compound (IC-1) (27 mg, 53 μmol, 91%, 98% Purity) as a clear, colourless solid. m/z 500.5 (M+H)⁺ (ES+). ¹H NMR (400 MHz, MeOD) δ 8.41 (s, 1H), 8.23 (dt, J=7.9, 1.3 Hz, 1H), 8.08 (t, J=1.8 Hz, 1H), 7.82 (dd, J=8.6, 5.5 Hz, 1H), 7.62-7.54 (m, 3H), 7.53-7.42 (m, 2H), 7.29 (s, 1H), 3.96 (s, 2H), 3.21 (s, 3H), 2.91 (s, 1H), 2.85 (s, 1H), 2.63 (s, 3H), 2.27 (dt, J=32.3, 10.7 Hz, 4H), 1.92-1.77 (m, 1H), 1.57-1.45 (m, 1H). Exchangeable NH proton not observed.

### Example 347: (1S,2R)-2-(((2-(4′-Fluoro-2′-(4-methyl-4H-1,2,4-triazol-3-yl)-[1,1′-biphenyl]-3-yl)-7-(trifluoromethyl)-1H-benzo[d]imidazol-5-yl)methyl)(methyl)amino)cyclopentan-1-ol (ID-3)

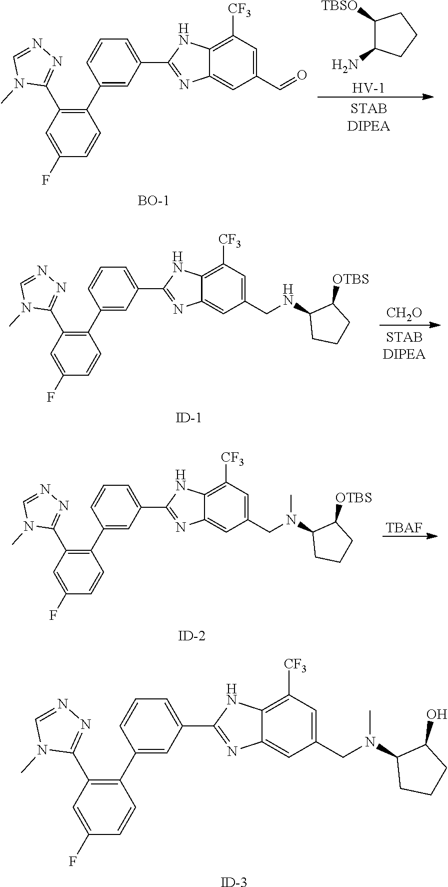

Step 1: (1R,2S)-2-((tert-Butyldimethylsilyl)oxy)-N-((2-(4′-fluoro-2′-(4-methyl-4H-1,2,4-triazol-3-yl)-[1,1′-biphenyl]-3-yl)-7-(trifluoromethyl)-1H-benzo[d]imidazol-5-yl)methyl)cyclopentan-1-amine (ID-1). To a solution of (1R,2S)-2-((tert-butyldimethylsilyl)oxy)cyclopentan-1-amine (HV-1) (270.1 mg, 33% Wt, 5.37 Eq, 413.8 μmol) (containing (1R,2S)-2-((tert-butyldimethylsilyl)oxy)-N-methylcyclopentan-1-amine (270.1 mg, 33% Wt, 5.04 Eq, 388.5 μmol) and (1R,2S)-2-((tert-butyldimethylsilyl)oxy)-N,N-dimethylcyclopentan-1-amine (270.1 mg, 33% Wt, 4.75 Eq, 366.1 μmol)) in CHCl₃ (3.0 mL) was added intermediate (BO-1) (47.2 mg, 76% Wt, 1 Eq, 77.1 μmol) and DIPEA (59 mg, 80 μL, 6.0 Eq, 0.46 mmol). The resulting mixture was stirred at rt for 1 h. NaBH(OAc)₃ (53.9 mg, 97% Wt, 3.20 Eq, 247 μmol) was added and the reaction mixture was stirred at rt overnight. NaBH(OAc)₃ (25.5 mg, 97% Wt, 1.51 Eq, 117 μmol) was added and the reaction mixture was stirred at rt for 1 h. The reaction mixture was diluted with EtOAc (5 mL), washed with sat. NaHCO₃ (5 mL) and brine (5 mL), dried (Na₂SO₄), filtered and concentrated in vacuo. The crude product was purified by chromatography on silica gel (12 g cartridge, 0-10% MeOH/DCM) to afford the sub-title compound (ID-1) (47.2 mg, 63 μmol, 82%, 89% Purity) as a pale-yellow solid. m/z 665.3 (M+H)⁺ (ES+). ¹H NMR (400 MHz, MeOD) δ 8.36 (s, 1H), 8.16 (s, 1H), 8.11 (d, J=7.9 Hz, 1H), 7.84-7.76 (m, 2H), 7.60 (s, 1H), 7.54 (td, J=8.5, 2.8 Hz, 1H), 7.50-7.42 (m, 2H), 7.23-7.17 (m, 1H), 4.33 (q, J=4.0 Hz, 1H), 4.29 (q, J=4.9 Hz, 1H), 3.41-3.36 (m, 1H), 3.20 (s, 3H), 3.06-3.01 (m, 1H), 2.09-1.99 (m, 1H), 1.98-1.91 (m, 1H), 1.89-1.75 (m, 2H), 1.75-1.56 (m, 2H), 0.95 (s, 9H), 0.15 (s, 6H). 2× Exchangeable NH protons not observed.

Step 2: (1R,2S)-2-((tert-Butyldimethylsilyl)oxy)-N-((2-(4′-fluoro-2′-(4-methyl-4H-1,2,4-triazol-3-yl)-[1,1′-biphenyl]-3-yl)-7-(trifluoromethyl)-1H-benzo[d]imidazol-5-yl)methyl)-N-methylcyclopentan-1-amine (ID-2). A solution of the product from step 1 above (ID-1) (45.2 mg, 89% Wt, 1 Eq, 60.5 μmol), formaldehyde solution in H₂O (11 mg, 10 μL, 37% Wt, 2.2 Eq, 0.13 mmol) and DIPEA (45 mg, 60 μL, 5.7 Eq, 0.34 mmol) in CHCl₃ (3.0 mL) was stirred at rt for 1 h. NaBH(OAc)₃ (78.4 mg, 97% Wt, 5.93 Eq, 359 μmol) was added and the reaction mixture was stirred at rt overnight. The reaction mixture was diluted with EtOAc (2 mL), washed with sat. NaHCO₃ (2 mL) and brine (2 mL), dried (Na₂SO₄), filtered and concentrated in vacuo. The crude product was purified by chromatography on silica gel (4 g cartridge, 0-10% MeOH/DCM) to afford the sub-title compound (ID-2) (22.8 mg, 31 μmol, 52%, 93% Purity) as an off-white solid. m/z 679.9 (M+H)⁺ (ES+). ¹H NMR (400 MHz, MeOD) δ 8.36 (s, 1H), 8.14 (s, 1H), 8.10 (d, J=7.9 Hz, 1H), 7.84-7.71 (m, 2H), 7.69-7.64 (m, 1H), 7.54 (td, J=8.5, 2.8 Hz, 1H), 7.50-7.42 (m, 2H), 7.20 (d, J=7.8 Hz, 1H), 4.43-4.38 (m, 1H), 3.93 (d, J=13.3 Hz, 1H), 3.78 (d, J=13.3 Hz, 1H), 3.20 (s, 3H), 2.65-2.57 (m, 1H), 2.24 (s, 3H), 1.95-1.86 (m, 3H), 1.84-1.75 (m, 1H), 1.75-1.58 (m, 2H), 0.96 (s, 9H), 0.13 (s, 3H), 0.12 (s, 3H). 1× Exchangeable NH proton not observed.

Step 3: (1S,2R)-2-(((2-(4′-Fluoro-2′-(4-methyl-4H-1,2,4-triazol-3-yl)-[1,1′-biphenyl]-3-yl)-7-(trifluoromethyl)-1H-benzo[d]imidazol-5-yl)methyl)(methyl)amino)cyclopentan-1-ol (ID-3). To a stirred solution of the product from step 2 above (ID-2) (22.8 mg, 93% Wt, 1 Eq, 31.2 μmol) in THF (1.5 mL) at rt was added TBAF in THF (90.8 mg, 100 μL, 1.0 molar, 3.20 Eq, 100 μmol). The reaction mixture was stirred at rt overnight. More TBAF in THF (52.3 mg, 200 μL, 1.0 molar, 6.40 Eq, 200 μmol) was added and the reaction mixture was stirred at rt for 3 h. Further TBAF in THF (78.4 mg, 300 μL, 1.0 molar, 9.60 Eq, 300 μmol) was added and the resulting mixture was stirred at rt overnight. The volatiles were removed in vacuo. The crude product was purified by chromatography on silica gel (4 g cartridge, 0-20% MeOH/DCM) to afford impure product (˜6 mg) and some recovered SM (˜17 mg). The recovered SM was desilylated with TBAF in THF (0.26 g, 1.0 mL, 1.0 molar, 32 Eq, 1.0 mmol) in THF (1.5 mL) and the mixture was stirred at rt overnight. The volatiles were removed in vacuo. The two crops of crude product were combined and purified by chromatography on silica gel (4 g cartridge, 0-20% MeOH/DCM) to afford impure product. The crude was purified by preparative HPLC (Waters, Basic (0.3% ammonia), Waters XBridge BEH C18 ODB, 5 μm, 30×100 mm column 35-100% MeCN in water) to afford the title compound (ID-3) (7.1 mg, 12 μmol, 38%, 95% Purity) as a white solid. m/z 565.8 (M+H)⁺ (ES+). ¹H NMR (400 MHz, MeOD) δ 8.36 (s, 1H), 8.14 (t, J=1.8 Hz, 1H), 8.10 (dt, J=8.0, 1.3 Hz, 1H), 7.84-7.77 (m, 2H), 7.60 (s, 1H), 7.54 (td, J=8.5, 2.8 Hz, 1H), 7.50-7.41 (m, 2H), 7.20 (ddd, J=7.8, 1.9, 1.1 Hz, 1H), 4.26 (t, J=3.7 Hz, 1H), 3.88 (d, J=13.1 Hz, 1H), 3.74 (d, J=13.2 Hz, 1H), 3.20 (s, 3H), 2.59 (ddd, J=11.0, 7.1, 3.7 Hz, 1H), 2.22 (s, 3H), 2.02-1.91 (m, 2H), 1.88-1.75 (m, 3H), 1.73-1.62 (m, 1H). 2× Exchangeable protons not observed.

### Example 348: 1-Cyclobutyl-N-((2-(4′-fluoro-2′-(4-methyl-4H-1,2,4-triazol-3-yl)-[1,1′-biphenyl]-3-yl)-7-(trifluoromethyl)benzo[d]oxazol-5-yl)methyl)-N-methylmethanamine (IE-1)

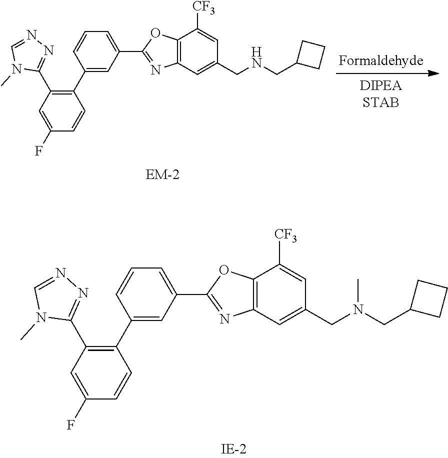 [SMILES: Cn1cnnc1-c1cc(F)ccc1-c1cccc(-c2nc3cc(CNCC4CCC4)cc(C(F)(F)F)c3o2)c1 ; CN(Cc1cc(C(F)(F)F)c2oc(-c3cccc(-c4ccc(F)cc4-c4nncn4C)c3)nc2c1)CC1CCC1]

A solution of compound (EM-2) (55 mg, 95% Wt, 1 Eq, 98 μmol), formaldehyde solution in H₂O (24 mg, 22 μL, 37% Wt, 3 Eq, 0.29 mmol) and DIPEA (63 mg, 85 μL, 5 Eq, 0.49 mmol) in CHCl₃ (1 mL) was stirred at 40° C. over the weekend. NaBH(OAc)₃ (0.10 g, 5 Eq, 0.49 mmol) was added and the reaction mixture was stirred at 40° C. for another 1 h. The reaction mixture was diluted with DCM (2 mL), washed with sat. NaHCO₃ (2 mL) and brine (2 mL), dried (Na₂SO₄), filtered and concentrated in vacuo. The crude product was purified by chromatography on silica gel (4 g cartridge, 0-10% MeOH/DCM) to afford the title compound (IE-1) (25 mg, 45 μmol, 46%, 99% Purity) as a white solid. m/z 550.5 (M+H)⁺ (ES+). ¹H NMR (400 MHz, MeOD) δ=8.41 (s, 1H), 8.24 (dt, J=7.9, 1.3 Hz, 1H), 8.09 (t, J=1.8 Hz, 1H), 7.98 (s, 1H), 7.81 (dd, J=8.7, 5.4 Hz, 1H), 7.74 (s, 1H), 7.64-7.54 (m, 2H), 7.53-7.44 (m, 2H), 3.73 (s, 2H), 3.27 (s, 3H), 2.67 (dt, J=15.1, 7.6 Hz, 1H), 2.53 (d, J=7.0 Hz, 2H), 2.27 (s, 3H), 2.14 (dddd, J=11.3, 8.1, 5.5, 2.4 Hz, 2H), 1.98 (dq, J=10.9, 9.0 Hz, 1H), 1.89-1.79 (m, 1H), 1.80-1.68 (m, 2H).

### Example 349: N-(2-(3,3-Difluoroazetidin-1-yl)ethyl)-2-(4′-fluoro-2′-(4-methyl-4H-1,2,4-triazol-3-yl)-[1,1′-biphenyl]-3-yl)-7-(trifluoromethyl)benzo[d]oxazol-5-amine (IF-2)

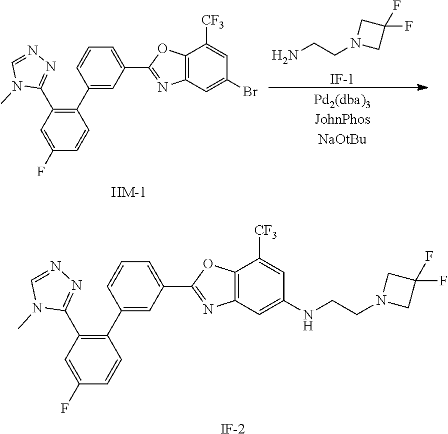

A stirred mixture of intermediate (HM-1) (50 mg, 1 Eq, 97 μmol), 2-(3,3-difluoroazetidin-1-yl)ethan-1-amine (IF-1) (33 mg, 2.5 Eq, 0.24 mmol), and NaOtBu (33 mg, 3.5 Eq, 0.34 mmol) and JohnPhos (5.8 mg, 0.2 eq, 0.019 mmol) in toluene (2 mL) was placed under three vacuum/N₂ cycles at 40° C. before Pd₂(dba)₃ (13 mg, 0.15 Eq, 14 μmol) was added. The mixture was degassed by another three vacuum/N₂ cycles before being warmed to 100° C. and stirring was continued for 3 h. The reaction mixture was diluted with distilled water (10 mL) and transferred into a separating funnel. The layer was extracted with DCM (3×10 mL). The combined extracted layers were collected, dried (Na₂SO₄), filtered and concentrated in vacuo. The crude product was purified by chromatography on silica gel (4 g cartridge, 0-10% (0.7 M ammonia/MeOH)/DCM) to afford the title compound (IF-2) (4 mg, 7 μmol, 7%, 99% Purity) as a pale-yellow solid. m/z 573.2 (M+H)⁺ (ES+). ¹H NMR (400 MHz, MeOD) δ=8.41 (s, 1H), 8.18 (dt, J=7.9, 1.3 Hz, 1H), 8.03 (t, J=1.7 Hz, 1H), 7.80 (dd, J=8.6, 5.5 Hz, 1H), 7.62-7.54 (m, 2H), 7.49 (dd, J=8.8, 2.7 Hz, 1H), 7.42 (ddd, J=7.8, 1.9, 1.1 Hz, 1H), 7.12 (d, J=2.3 Hz, 1H), 7.03 (dd, J=2.3, 0.9 Hz, 1H), 3.73 (t, J=12.1 Hz, 4H), 3.26 (d, J=1.8 Hz, 5H), 2.96-2.87 (m, 2H). Exchangeable NH not observed.

### Example 350: (1S,2R)-2-(((2-(4′-Fluoro-2′-(1,3,4-oxadiazol-2-yl)-[1,1′-biphenyl]-3-yl)-7-(trifluoromethyl)benzo[d]oxazol-5-yl)methyl)amino)cyclopentan-1-ol (IG-6)

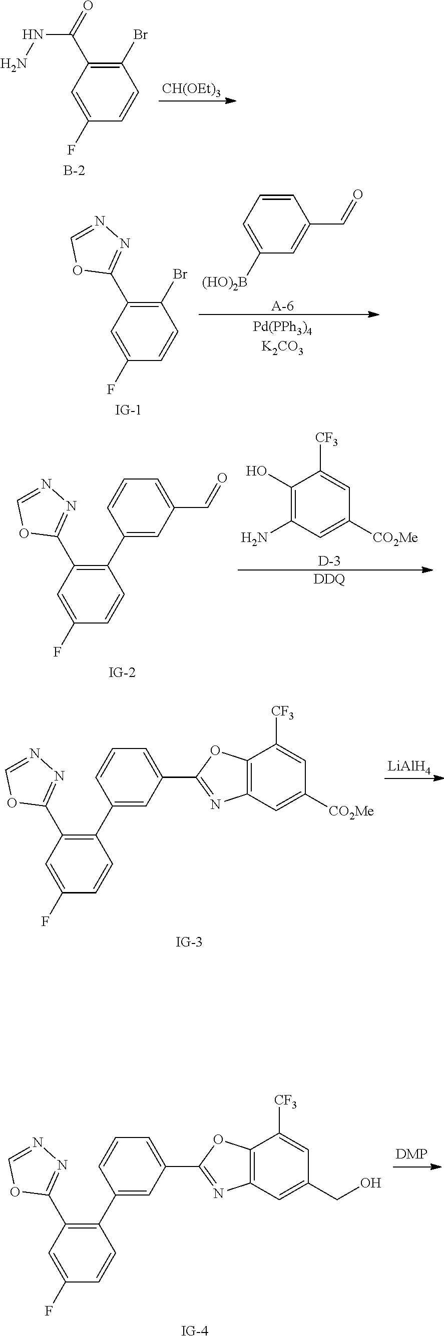

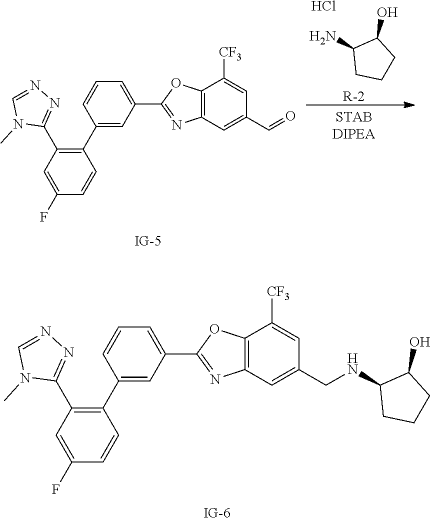

Step 1: 2-(2-Bromo-5-fluorophenyl)-1,3,4-oxadiazole (IG-1). 2-Bromo-5-fluorobenzohydrazide (B-2) (215.9 mg, 1 Eq, 926.5 μmol) and triethyl orthoformate (0.9 g, 1 mL, 6 Eq, 6 mmol) were stirred at 160° C. for 16 h. The reaction mixture was concentrated in vacuo. The residue was dissolved in DCM (10 mL) and washed with 1 M HCl (10 mL) and brine (10 mL). The organic layer was extracted, dried (phase separator) and concentrated in vacuo. The crude product was purified by chromatography on silica gel (12 g cartridge, 0-100% EtOAc/isohexane) to afford the sub-title compound (IG-1) (129.8 mg, 0.52 mmol, 56%, 98% Purity) as a white solid. ¹H NMR (400 MHz, DMSO) δ 9.51 (s, 1H), 7.94 (dd, J=8.9, 5.2 Hz, 1H), 7.83 (dd, J=9.0, 3.1 Hz, 1H), 7.50 (ddd, J=9.0, 8.1, 3.1 Hz, 1H).

Step 2: 4′-Fluoro-2′-(1,3,4-oxadiazol-2-yl)-[1,1′-biphenyl]-3-carbaldehyde (IG-2). A solution of the product from step 1 above (IG-1) (129.8 mg, 90% Wt, 1 Eq, 480.7 μmol), (3-formylphenyl)boronic acid (A-6) (79.28 mg, 1.1 Eq, 528.7 μmol) and K₂CO₃ (199.3 mg, 3 Eq, 1.442 mmol) in dioxane (4 mL) and water (1.25 mL) was degassed for 10 min. Pd(PPh₃)₄ (13.89 mg, 0.025 Eq, 12.02 μmol) was added and the resulting mixture was heated at 80° C. under a nitrogen atmosphere for 16 h. The reaction mixture was concentrated in vacuo. The residue was dissolved in DCM (10 mL) and washed with water (2×10 mL). The organic layer was separated, dried (phase separator) and concentrated in vacuo. The crude product was purified by chromatography on silica gel (12 g cartridge, 0-10% MeOH/DCM) to afford the sub-title compound (IG-2) (105.6 mg, 0.35 mmol, 74%, 90% Purity) as a dark yellow oil. m/z 269.0 (M+H)⁺ (ES+). ¹H NMR (400 MHz, DMSO) δ 10.02 (s, 1H), 9.20 (s, 1H), 7.93 (dt, J=7.6, 1.5 Hz, 1H), 7.87-7.82 (m, 1H), 7.78 (t, J=1.8 Hz, 1H), 7.67-7.59 (m, 3H), 7.58-7.54 (m, 1H).

Step 3: Methyl 2-(4′-fluoro-2′-(1,3,4-oxadiazol-2-yl)-[1,1′-biphenyl]-3-yl)-7-(trifluoromethyl)benzo[d]oxazole-5-carboxylate (IG-3). To a solution of the product from step 2 above (IG-2) (105.6 mg, 90% Wt, 1 Eq, 354.3 μmol) in MeOH (5 mL) was added methyl 3-amino-4-hydroxy-5-(trifluoromethyl)benzoate (D-3) (87.70 mg, 95% Wt, 1 Eq, 354.3 μmol) and the reaction was stirred at 45° C. for 16 h. After concentration in vacuo, the residue was dissolved in DCM (5 mL) and DDQ (88.5 mg, 1.1 Eq, 389.7 μmol) was then added. After stirring at rt for 1 h, the resulting mixture was diluted with DCM (20 mL) and washed sequentially with sat. aq. sol. of NaHCO₃ (2×20 mL) and brine (2×20 mL). The organic layer was dried (phase separator) and the solvent was removed in vacuo. The crude product was purified by chromatography on silica gel (12 g cartridge, 0-10% MeOH/DCM) to afford the sub-title compound (IG-3) (168.1 mg, 0.31 mmol, 88%, 90% Purity) as a dark brown solid. m/z 484.0 (M+H)⁺ (ES+).

Step 4: (2-(4′-Fluoro-2′-(1,3,4-oxadiazol-2-yl)-[1,1′-biphenyl]-3-yl)-7-(trifluoromethyl)benzo[d]oxazol-5-yl)methanol (IG-4). To a solution of the product from step 3 above (IG-3) (168.1 mg, 90% Wt, 1 Eq, 313.0 μmol) in THF (10 mL) was added LiAlH₄ (13.07 mg, 0.3443 mL, 1 molar, 1.100 Eq, 344.3 μmol) dropwise at 0° C. This mixture was stirred 1 h at this temperature. The mixture was quenched with H₂O (4 mL), and NaOH (aq. 15%, 4 mL) was added at 0° C. MgSO₄ was added and the mixture was warmed up to rt and stirred for 15 min. The mixture was filtered and the solvent was removed in vacuo. The crude product was purified by chromatography on silica gel (12 g cartridge, 0-10% MeOH/DCM) to afford the sub-title compound (IG-4) (28.1 mg, 59 μmol, 19%, 95% Purity) as a pale brown solid. m/z 456.5 (M+H)⁺ (ES+).

Step 5: 2-(4′-fluoro-2′-(1,3,4-oxadiazol-2-yl)-[1,1′-biphenyl]-3-yl)-7-(trifluoromethyl)benzo[d]oxazole-5-carbaldehyde (IG-5). The product from step 4 above (IG-4) (28.1 mg, 1 Eq, 61.7 μmol) and Dess-Martin periodinane (39.3 mg, 1.5 Eq, 92.6 μmol) were stirred in DCM (2 mL) at rt for 1 h. The reaction mixture was diluted with DCM (5 mL) and washed with sat. aq. NaHCO₃ (10 mL) and brine (10 mL). Organics were separated, dried (phase separator), and evaporated in vacuo. The crude product was purified by chromatography on silica gel (4 g cartridge, 0-10% MeOH/DCM) to afford the sub-title compound (IG-5) (30 mg, 63 μmol, 100%, 95% Purity) as a pale tan solid. m/z 454.0 (M+H)⁺ (ES+).

Step 6: (1S,2R)-2-(((2-(4′-Fluoro-2′-(1,3,4-oxadiazol-2-yl)-[1,1′-biphenyl]-3-yl)-7-(trifluoromethyl)benzo[d]oxazol-5-yl)methyl)amino)cyclopentan-1-ol (IG-6). A solution of the product from step 5 above (IG-5) (30 mg, 90% Wt, 1 Eq, 60 μmol), (1S,2R)-2-aminocyclopentan-1-ol, HCl (R-2) (16 mg, 2.0 Eq, 0.12 mmol) and DIPEA (23 mg, 31 μL, 3.0 Eq, 0.18 mmol) in CHCl₃ (5 mL) was stirred 1 h at rt, then NaBH(OAc)₃ (38 mg, 3.0 Eq, 0.18 mmol) was added and the reaction mixture was stirred overnight at rt. The reaction mixture was diluted with EtOAc (25 mL) and washed with sat. aq. sol. of NaHCO₃ (2×10 mL) and brine (10 mL). The organic layer was collected, dried (MgSO₄), filtered and concentrated in vacuo. The crude product was purified by chromatography on silica gel (12 g cartridge, 0-10% MeOH/DCM) to afford the title compound (IG-6) (7.62 mg, 13 μmol, 22%, 94% Purity) as a white solid. m/z 539.4 (M+H)⁺ (ES+). ¹H NMR (400 MHz, DMSO) δ 9.20 (s, 1H), 8.22 (dt, J=7.9, 1.4 Hz, 1H), 8.11 (s, 1H), 8.00 (t, J=1.8 Hz, 1H), 7.87 (dd, J=9.3, 2.8 Hz, 1H), 7.82 (s, 1H), 7.75-7.63 (m, 3H), 7.54 (dt, J=7.7, 1.5 Hz, 1H), 4.02-3.90 (m, 3H), 2.84 (q, J=7.5 Hz, 1H), 1.76-1.56 (m, 3H), 1.51-1.36 (m, 3H). Two exchangeable protons not observed.

### Example 351: (1S,2R)-2-(((2-(4′-Fluoro-2′-(4-methyl-4H-1,2,4-triazol-3-yl)-[1,1′-biphenyl]-3-yl)-8-methoxy-[1,2,4]triazolo[1,5-a]pyridin-6-yl)methyl)amino)cyclopentan-1-ol (IH-9)

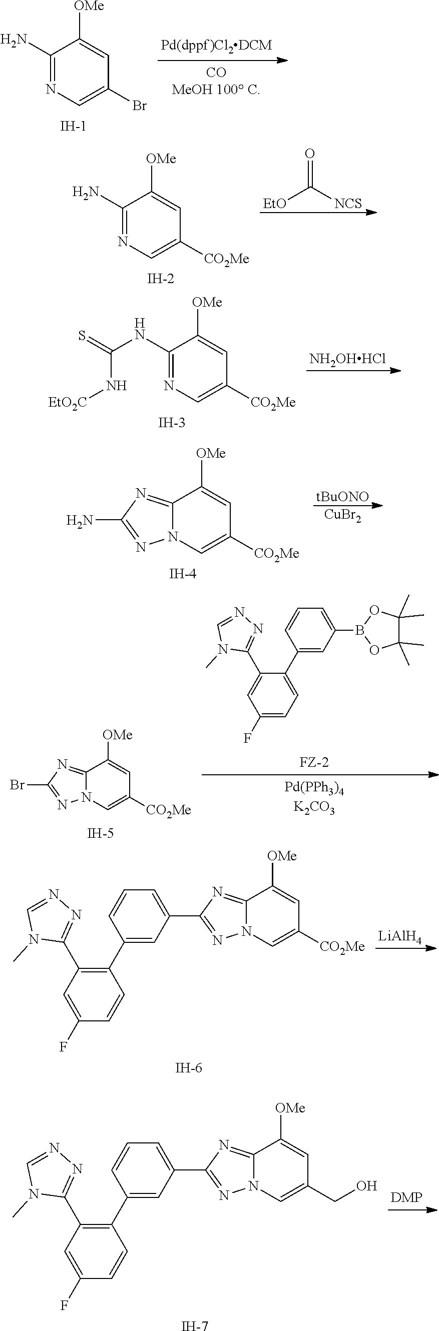

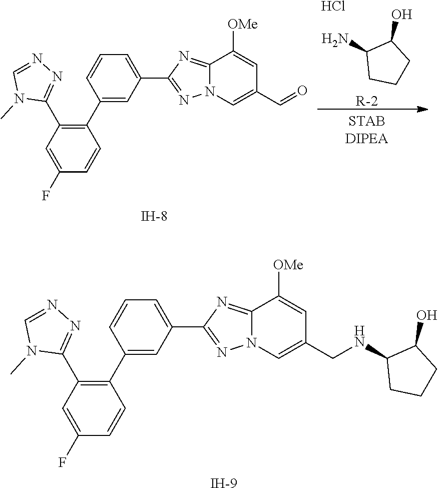

Step 1: Methyl 6-amino-5-methoxynicotinate (IH-2). To a solution of 5-bromo-3-methoxypyridin-2-amine (IH-1) (0.5 g, 1 Eq, 2 mmol) and Pd(dppf)Cl₂-DCM (0.1 g, 0.05 Eq, 0.1 mmol) in MeOH (10 mL) and DMF (2 mL) was added Et₃N (1 g, 1 mL, 4 Eq, 0.01 mol). The vessel was sealed, purged with nitrogen three times, purged with CO three times and pressurized to 5 bar with CO. The reaction mixture was heated to 100° C. and stirred for 12 h. The vessel was cooled to rt and was purged with nitrogen three times. The reaction mixture was then concentrated in vacuo to give a crude brown residue. The crude product was purified by chromatography on silica gel (24 g cartridge, 0-10% MeOH/DCM) to afford the sub-title compound (IH-2) (300 mg, 1.6 mmol, 60%, 97% Purity) as a light brown solid. m/z 183.1 (M+H)⁺ (ES+). ¹H NMR (400 MHz, DMSO) δ=8.18 (d, J=1.8 Hz, 1H), 7.30 (d, J=1.9 Hz, 1H), 6.66 (s, 2H), 3.82 (s, 3H), 3.78 (s, 3H).

Step 2: Methyl 6-(3-(ethoxycarbonyl)thioureido)-5-methoxynicotinate (IH-3). To a stirred solution of the product from step 1 above (IH-2) (300 mg, 1 Eq, 1.65 mmol) in dry 1,4-dioxane (5 mL) under a nitrogen atmosphere at 25° C. was added O-ethyl carbonisothiocyanatidate (259 mg, 212 μL, 1.2 Eq, 1.98 mmol). The reaction mixture was stirred for 12 h. The crude was dried in vacuo and washed with DCM/pentane (1:9) (50 mL) to afford the sub-title compound (IH-3) (430 mg, 1.3 mmol, 82%, 98% Purity) as a pale brown solid. m/z 314.2 (M+H)⁺ (ES+). ¹H NMR (400 MHz, DMSO) δ=11.70 (s, 1H), 11.43 (s, 1H), 8.59-8.44 (m, 1H), 7.85 (t, J=2.6 Hz, 1H), 4.28-4.11 (m, 2H), 3.93 (d, J=2.4 Hz, 3H), 3.89 (d, J=3.7 Hz, 3H), 1.31-1.19 (m, 3H).

Step 3: Methyl 2-amino-8-methoxy-[1,2,4]triazolo[1,5-a]pyridine-6-carboxylate (IH-4). To a stirred solution of the product from step 2 above (IH-3) (430 mg, 1 Eq, 1.37 mmol) in dry EtOH (5 mL) under a nitrogen atmosphere at 90° C. was added NH₂OH, HCl (477 mg, 5 Eq, 6.86 mmol) and DIPEA (532 mg, 717 μL, 3 Eq, 4.12 mmol). The reaction mixture was stirred for 12 h. The bulk solvent was removed in vacuo. The reaction mixture was diluted with distilled water (25 mL) and transferred into a separating funnel. The layer was extracted with DCM/MeOH (5:1) (3×25 mL). The combined extracted layers were collected, dried (Na₂SO₄), filtered and concentrated in vacuo. The crude product was purified by chromatography on silica gel (24 g cartridge, 0-10% MeOH/DCM) to afford the sub-title compound (IH-4) (260 mg, 1.2 mmol, 84%, 99% Purity) as a dark brown solid. m/z 223.2 (M+H)⁺ (ES+). ¹H NMR (400 MHz, DMSO) δ=8.69 (d, J=1.3 Hz, 1H), 7.21 (d, J=1.4 Hz, 1H), 6.28 (s, 2H), 3.96 (s, 3H), 3.87 (s, 3H).

Step 4: Methyl 2-bromo-8-methoxy-[1,2,4]triazolo[1,5-a]pyridine-6-carboxylate (IH-5). To a stirred solution of CuBr₂ (754 mg, 3 Eq, 3.38 mmol) in MeCN (4 mL) was added tert-butyl nitrite (348 mg, 3 Eq, 3.38 mmol). The reaction mixture was heated to 55° C. for 10 min. The product from step 3 above (IH-4) (250 mg, 1 Eq, 1.13 mmol) was added portion wise, and the reaction mixture was heated at 55° C. for 4 h. The reaction mixture was diluted with distilled water (25 mL) and sonicated. Then the mixture was filtered, and the residue was washed thoroughly with Et₂O (3×10 mL) and concentrated in vacuo. The crude product was purified by chromatography on silica gel (12 g cartridge, 0-10% MeOH/DCM) to afford the sub-title compound (IH-5) (230 mg, 0.79 mmol, 70%, 98% Purity) as a pale brown solid. m/z 286.1 (M+H)⁺ (ES+). ¹H NMR (400 MHz, DMSO) δ=9.10 (d, J=1.3 Hz, 1H), 7.43 (d, J=1.3 Hz, 1H), 4.05 (s, 3H), 3.92 (s, 3H).

Step 5: Methyl 2-(4′-fluoro-2′-(4-methyl-4H-1,2,4-triazol-3-yl)-[1,1′-biphenyl]-3-yl)-8-methoxy-[1,2,4]triazolo[1,5-a]pyridine-6-carboxylate (IH-6). K₂CO₃ (39 mg, 3 Eq, 0.28 mmol) in water (0.3 mL) was added to a mixture of intermediate (FZ-2) (36 mg, 1 Eq, 94 μmol), the product from step 4 above (IH-5) (27 mg, 1 Eq, 94 μmol) and Pd(PPh₃)₄ (11 mg, 0.1 Eq, 9.4 μmol) in toluene (1.3 mL). The mixture was then stirred at 110° C. (microwave) for 1.5 h. The reaction mixture was diluted with distilled water (5 mL) and transferred into a separating funnel. The layer was extracted with DCM (3×10 mL). The combined extracted layers were collected, dried (Na₂SO₄), filtered and concentrated in vacuo. The crude product was purified by chromatography on silica gel (12 g cartridge, 0-10% MeOH/DCM) to afford the sub-title compound (IH-6) (28 mg, 60 μmol, 64%, 99% Purity) as a clear white solid. m/z 459.1 (M+H)⁺ (ES+).

Step 6: 2-(4′-Fluoro-2′-(4-methyl-4H-1,2,4-triazol-3-yl)-[1,1′-biphenyl]-3-yl)-8-methoxy-[1,2,4]triazolo[1,5-a]pyridin-6-yl)methanol (IH-7). To a solution of the product from step 5 above (IH-6) (28 mg, 1 Eq, 61 μmol) in THF (2 mL) was added LiAlH₄ (2.5 mg, 67 μL, 1 molar, 1.1 Eq, 67 μmol) dropwise at 0° C. This mixture was stirred 2 h at this temperature. The reaction mixture was diluted with distilled water (10 mL) and acidified with 2 mL 1M HCl and transferred into a separating funnel. The layer was extracted with DCM (3×10 mL). The combined extracted layers were collected, dried (Na₂SO₄), filtered and concentrated in vacuo. The crude product was purified by chromatography on silica gel (40 g cartridge, 0-10% (0.7 M ammonia/MeOH)/DCM) to afford the sub-title compound (IH-7) (27 mg, 56 μmol, 91%, 89% Purity) as a thick colourless solid. m/z 431.2 (M+H)⁺ (ES+).

Step 7: 2-(4′-Fluoro-2′-(4-methyl-4H-1,2,4-triazol-3-yl)-[1,1′-biphenyl]-3-yl)-8-methoxy-[1,2,4]triazolo[1,5-a]pyridine-6-carbaldehyde (IH-8). To a stirred solution of the product from step 6 above (IH-7) (28 mg, 60% Wt, 1 Eq, 39 μmol) in dry DCM (2 mL) under a nitrogen atmosphere at 25° C. was added DMP (33 mg, 2 Eq, 78 μmol). The reaction mixture was stirred for 1 h. The reaction mixture was diluted with DCM (10 mL) and washed with sat. aq. NaHCO₃ (10 mL) and brine. Organics were separated, dried (MgSO₄), filtered and evaporated in vacuo. The crude product was purified by chromatography on silica gel (4 g cartridge, 0-10% (0.7 M ammonia/MeOH)/DCM) to afford the sub-title compound (IH-8) (15 mg, 35 μmol, 90%, 100% Purity) as a pale yellow solid. m/z 429.0 (M+H)⁺ (ES+).

Step 8: (1S,2R)-2-(((2-(4′-Fluoro-2′-(4-methyl-4H-1,2,4-triazol-3-yl)-[1,1′-biphenyl]-3-yl)-8-methoxy-[1,2,4]triazolo[1,5-a]pyridin-6-yl)methyl)amino)cyclopentan-1-ol (IH-9). A solution of the product from step 7 above (IH-8) (15 mg, 96% Wt, 1 Eq, 34 μmol), (1S,2R)-2-aminocyclopentan-1-ol, HCl (R-2) (12 mg, 75% Wt, 2 Eq, 67 μmol) and DIPEA (22 mg, 29 μL, 5 Eq, 0.17 mmol) in CHCl₃ (1 mL) was stirred at 45° C. for 12 h. NaBH(OAc)₃ (36 mg, 5 Eq, 0.17 mmol) was added and the reaction mixture was stirred at 45° C. for another 2 h. The reaction mixture was diluted with Sat. NaHCO₃ (10 mL) and transferred into a separating funnel. The layer was extracted with DCM (3×10 mL). The combined extracted layers were collected, dried (Na₂SO₄), filtered and concentrated in vacuo. The crude product was purified by chromatography on silica gel (4 g cartridge, 0-10% MeOH/DCM) to afford the title compound (IH-9) (8 mg, 0.02 mmol, 40%, 97% Purity) as a light-yellow solid. m/z 514.2 (M+H)⁺ (ES+). ¹H NMR (400 MHz, MeOD) δ=8.46 (s, 1H), 8.38 (s, 1H), 8.23-8.18 (m, 1H), 8.17 (d, J=1.9 Hz, 1H), 7.80 (dd, J=8.7, 5.5 Hz, 1H), 7.56 (td, J=8.5, 2.8 Hz, 1H), 7.51-7.44 (m, 2H), 7.29-7.21 (m, 2H), 4.24-4.20 (m, 1H), 4.14 (s, 3H), 4.01 (q, J=13.4 Hz, 2H), 3.17 (s, 3H), 3.13-3.05 (m, 1H), 1.92-1.82 (m, 3H), 1.82-1.73 (m, 1H), 1.67-1.59 (m, 2H). Exchangeable NH & OH not observed.

### Example 352: N-((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)acetamide (II-1)

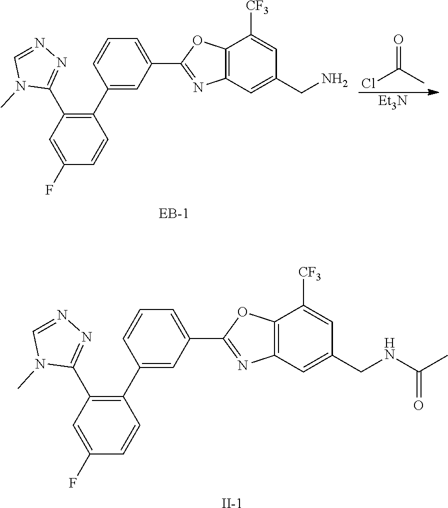

To a crude solution of intermediate (EB-1) dissolved in MeCN (2 mL) was added acetyl chloride (17.4 mg, 15.8 μL, 1.4 Eq, 222 μmol) and Et₃N (48.1 mg, 66.3 μL, 3 Eq, 476 μmol). The resulting mixture was stirred at rt overnight. The reaction mixture was concentrated in vacuo. The crude product was purified by chromatography on silica gel (12 g cartridge, 0-100% MeOH/DCM) to afford the title compound (II-1) (4.05 mg, 5%) as a pale-yellow solid. m/z 510.1 (M+H)⁺ (ES+). ¹H NMR (400 MHz, DMSO) δ 8.54-8.49 (m, 1H), 8.42 (s, 1H), 8.13 (d, J=8.0 Hz, 1H), 7.99 (s, 1H), 7.92-7.89 (m, 1H), 7.81-7.74 (m, 1H), 7.69 (s, 1H), 7.64-7.52 (m, 3H), 7.38 (d, J=8.0 Hz, 1H), 4.44 (d, J=5.9 Hz, 2H), 3.21 (s, 3H), 1.91 (s, 3H).

### Example 353: 1-((((2-(4′-Fluoro-2′-(4-methyl-4H-1,2,4-triazol-3-yl)-[1,1′-biphenyl]-3-yl)-7-methylbenzo[d]oxazol-5-yl)methyl)amino)methyl)cyclobutan-1-ol (IJ-1)

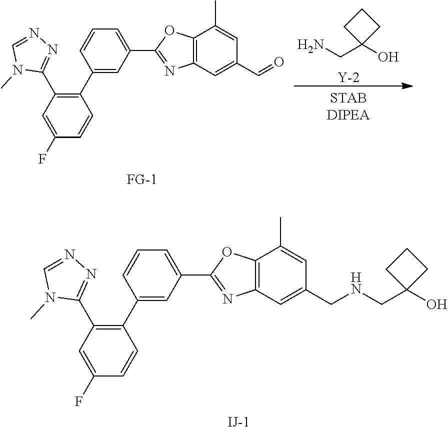

NaBH(OAc)₃ (51 mg, 3.0 Eq, 0.24 mmol) was added to a stirred mixture of intermediate (FG-1) (35 mg, 95% Wt, 1 Eq, 81 μmol), 1-(aminomethyl)cyclobutan-1-ol (Y-2) (16 mg, 2.0 Eq, 0.16 mmol) and DIPEA (31 mg, 43 μL, 3.0 Eq, 0.24 mmol) in CHCl₃ (3 mL). The reaction mixture was stirred at 40° C. overnight. The reaction mixture was diluted with DCM (4 mL) and washed with sat. aq. NaHCO₃ (10 mL). The organic phase was separated and evaporated to afford the crude product. The crude product was purified by preparative HPLC (Waters, Basic (0.3% ammonium bicarbonate), Basic, Waters XBridge BEH C18 ODB, 5 μm, 30×100 mm column, 30-100% MeCN in water) to afford the title compound (IJ-1) (15 mg, 30 μmol, 37%, 98% Purity) as a white solid. m/z 497.9 (M+H)⁺ (ES+). ¹H NMR (400 MHz, DMSO) δ 8.43 (s, 1H), 8.13 (dt, J=7.9, 1.4 Hz, 1H), 7.92 (t, J=1.8 Hz, 1H), 7.79 (dd, J=8.6, 5.7 Hz, 1H), 7.60 (td, J=8.6, 2.8 Hz, 1H), 7.57-7.50 (m, 3H), 7.33 (dt, J=8.0, 1.4 Hz, 1H), 7.24 (s, 1H), 4.88 (s, 1H), 3.83 (s, 2H), 3.29 (s, 1H), 3.15 (s, 3H), 2.54 (s, 3H), 1.99 (td, J=9.1, 4.7 Hz, 2H), 1.89 (dt, J=11.8, 9.2 Hz, 2H), 1.60 (d, J=10.7 Hz, 1H), 1.39 (dt, J=10.9, 8.8 Hz, 1H). Missing CH₂ under solvent peak.

### Example 354: 1-(((2-(4′-Fluoro-2′-(4-methyl-4H-1,2,4-triazol-3-yl)-[1,1′-biphenyl]-3-yl)-7-methylbenzo[d]oxazol-5-yl)methyl)amino)-2-methylpropan-2-ol (IK-1)

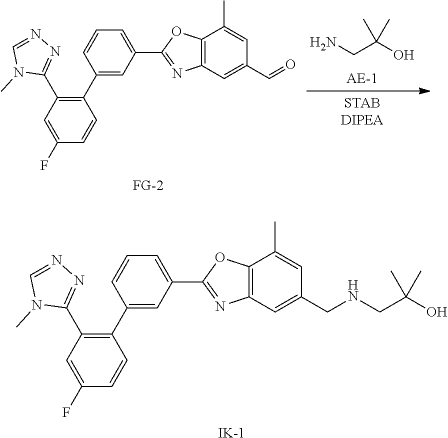

NaBH(OAc)₃ (51 mg, 3.0 Eq, 0.24 mmol) was added to a stirred mixture of intermediate (FG-1) (35 mg, 95% Wt, 1 Eq, 81 μmol), 1-amino-2-methylpropan-2-ol (AE-1) (14 mg, 2.0 Eq, 0.16 mmol) and DIPEA (31 mg, 43 μL, 3.0 Eq, 0.24 mmol) in CHCl₃ (3 mL). The reaction mixture was stirred at 40° C. overnight. The reaction mixture was diluted with DCM (4 mL) and washed with sat. aq. NaHCO₃ (10 mL). The organic phase was separated and evaporated to afford the crude product. The crude product was purified by preparative HPLC (Waters, Basic (0.3% ammonium bicarbonate), Basic Waters XBridge BEH C18 ODB, 5 μm, 30×100 mm column 25-100% MeCN in water, to afford the title compound (IK-1) (14 mg, 28 μmol, 35%, 97% Purity) as a white solid. m/z 486.7 (M+H)⁺ (ES+). ¹H NMR (400 MHz, DMSO) δ 8.43 (s, 1H), 8.13 (dt, J=7.9, 1.3 Hz, 1H), 7.92 (t, J=1.8 Hz, 1H), 7.79 (dd, J=8.7, 5.7 Hz, 1H), 7.60 (td, J=8.5, 2.8 Hz, 1H), 7.57-7.50 (m, 3H), 7.33 (dt, J=7.9, 1.4 Hz, 1H), 7.23 (s, 1H), 4.19 (s, 1H), 3.81 (s, 2H), 3.29 (s, 1H), 3.15 (s, 3H), 2.54 (s, 3H), 2.38 (s, 2H), 1.10 (s, 6H).

### Example 355: rac-N-(Cyclobutylmethyl)-2,2,2-trifluoro-1-(7-fluoro-2-(4′-fluoro-2′-(4-methyl-4H-1,2,4-triazol-3-yl)-[1,1′-biphenyl]-3-yl)benzo[d]oxazol-5-yl)ethan-1-amine (IL-6)

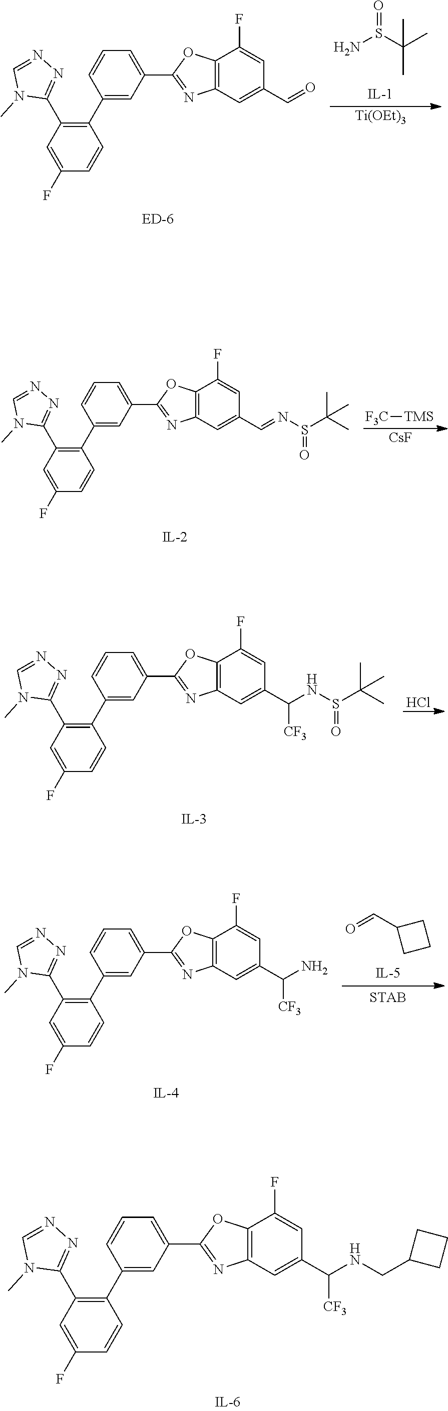

Step 1: (E)-N-((7-Fluoro-2-(4′-fluoro-2′-(4-methyl-4H-1,2,4-triazol-3-yl)-[1,1′-biphenyl]-3-yl)benzo[d]oxazol-5-yl)methylene)-2-methylpropane-2-sulfinamide (IL-2). To a stirred solution of intermediate (ED-6) (115 mg, 98% Wt, 1 Eq, 271 μmol) in dry THF (2 mL) under a nitrogen atmosphere at 60° C. was added 2-Methyl-2-propanesulfinamide (IL-1) (52.5 mg, 1.6 Eq, 433 μmol) and Ti(OEt)₃ (185 mg, 169 μL, 3 Eq, 812 μmol) were added. The reaction mixture was stirred for 12 h. The reaction mixture was concentrated in vacuo. The reaction mixture was dissolved in EtOAc and diluted with potassium sodium tartrate sat. solution (10 mL) and transferred into a separating funnel. The layer was extracted with EtOAc (3×10 mL). The combined extracted layers were collected, dried (Na₂SO₄), filtered and concentrated in vacuo. The crude product was purified by chromatography on silica gel (4 g cartridge, 0-10% MeOH/DCM) to afford the sub-title compound (IL-2) (95 mg, 0.16 mmol, 60%, 89% Purity). m/z 520.1 (M+H)⁺ (ES+).

Step 2: rac-2-Methyl-N-(2,2,2-trifluoro-1-(7-fluoro-2-(4′-fluoro-2′-(4-methyl-4H-1,2,4-triazol-3-yl)-[1,1′-biphenyl]-3-yl)benzo[d]oxazol-5-yl)ethyl)propane-2-sulfinamide (IL-3). To a stirred solution of the product from step 1 above (IL-2) (90 mg, 1 Eq, 0.17 mmol) and CsF (0.18 g, 7 Eq, 1.2 mmol) in dry DME (2 mL) under a nitrogen atmosphere at 0° C. was slowly added trifluoromethyltrimethylsilane (0.17 g, 0.18 mL, 7 Eq, 1.2 mmol). The reaction mixture was stirred at 0° C. for 30 min and slowly warmed to 25° C. for 2 h. The reaction mixture was diluted with brine (10 mL) and transferred into a separating funnel. The layer was extracted with EtOAc (3×10 mL). The combined extracted layers were collected, dried (Na₂SO₄), filtered and concentrated in vacuo. The crude product was purified by chromatography on silica gel (4 g cartridge, 0-10% MeOH/DCM) to afford the sub-title compound (IL-3) (65 mg, 99 μmol, 57%, 90% Purity) as a pale-yellow solid. m/z 590.1 (M+H)⁺ (ES+).

Step 3: rac-2,2,2-Trifluoro-1-(7-fluoro-2-(4′-fluoro-2′-(4-methyl-4H-1,2,4-triazol-3-yl)-[1,1′-biphenyl]-3-yl)benzo[d]oxazol-5-yl)ethan-1-amine (IL-4). To a stirred solution of the product from step 2 above (IL-3) (65 mg, 90% Wt, 1 Eq, 99 μmol) in dry MeOH (2 mL) under a nitrogen atmosphere at 25° C. was added HCl (9.0 mg, 62 μL, 4 molar, 2.5 Eq, 0.25 mmol). The reaction mixture was stirred for 2 h. After 2 h another batch of HCl (9.0 mg, 62 μL, 4 molar, 2.5 Eq, 0.25 mmol) was added and continue the stirring for 1 h. After completion of reaction the reaction mixture was concentrated in vacuo at rt affording the sub-title compound (IL-4) (51 mg, 98 μmol, 98%, 93% Purity) as a pale-yellow solid. m/z 486.1 (M+H)⁺ (ES+).

Step 4: rac-N-(Cyclobutylmethyl)-2,2,2-trifluoro-1-(7-fluoro-2-(4′-fluoro-2′-(4-methyl-4H-1,2,4-triazol-3-yl)-[1,1′-biphenyl]-3-yl)benzo[d]oxazol-5-yl)ethan-1-amine (IL-6). A solution of the product from step 3 above (IL-4) (30 mg, 96% Wt, 1 Eq, 59 μmol), cyclobutanecarbaldehyde (IL-5) (15 mg, 3 Eq, 0.18 mmol) and DIPEA (38 mg, 52 μL, 5 Eq, 0.30 mmol) in CHCl₃ (1 mL) was stirred at 45° C. for 12 h. NaBH(OAc)₃ (63 mg, 5 Eq, 0.30 mmol) was added and heated at 45° C. for additional 2 h. The reaction mixture was diluted with distilled water (5 mL) and transferred into a separating funnel. The layer was extracted with DCM (3×5 mL). The combined extracted layers were collected, dried (Na₂SO₄), filtered and concentrated in vacuo. The crude product was purified by chromatography on silica gel (4 g cartridge, 0-10% MeOH/DCM) to afford the title compound (IL-6) (12 mg, 21 μmol, 36%, 98% Purity) as a clear white solid. m/z 554.1 (M+H)⁺ (ES+). ¹H NMR (400 MHz, MeOD) δ=8.41 (s, 1H), 8.26 (d, J=8.0 Hz, 1H), 8.14 (t, J=1.9 Hz, 1H), 7.82 (dd, J=8.6, 5.4 Hz, 1H), 7.74 (s, 1H), 7.59 (dt, J=11.6, 8.1 Hz, 2H), 7.52-7.42 (m, 3H), 4.47 (d, J=7.6 Hz, 1H), 3.23 (s, 3H), 2.68-2.44 (m, 4H), 2.08 (dq, J=7.8, 3.8 Hz, 2H), 1.98-1.89 (m, 1H), 1.67 (s, 2H). Exchangeable NH Not observed.

### Example 356: rac-(1S,2R)-2-(((2-(4′-fluoro-2′-(4-methyl-4H-1,2,4-triazol-3-yl)-[1,1′-biphenyl]-3-yl)-7-(trifluoromethyl)benzo[d]oxazol-5-yl)methyl)amino)-2-methylcyclopentan-1-ol (IM-8)

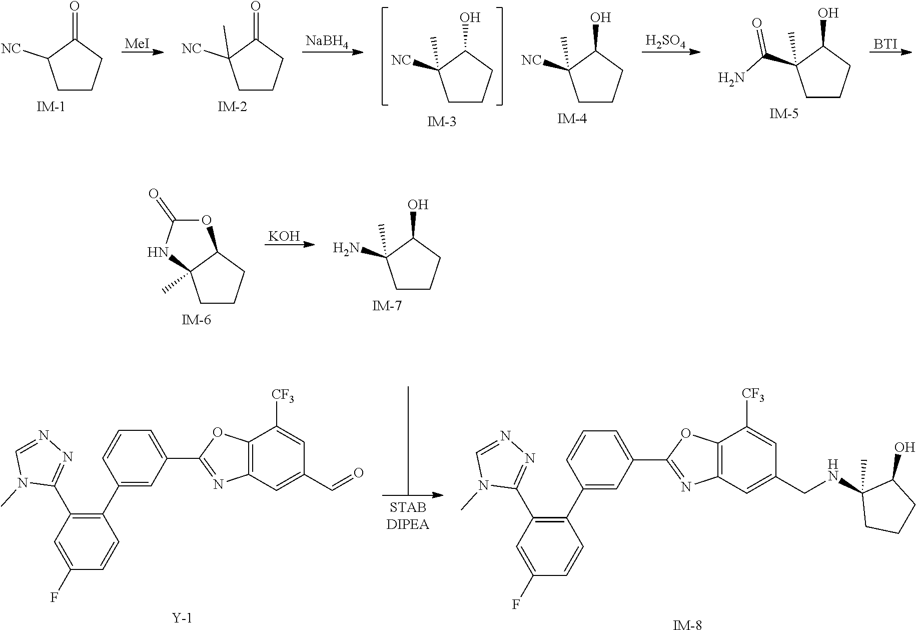

Step 1: 1-Methyl-2-oxocyclopentane-1-carbonitrile (IM-2). To a stirred solution of cyclopentan-1-one-2-carbonitrile (IM-1) (2.04 g, 97% Wt, 1 Eq, 18.1 mmol) in MeOH (10 mL) at rt was added LiOH (571.1 mg, 1.32 Eq, 23.85 mmol) in water (10 mL) and Mel (3.12 g, 1.42 mL, 1.21 Eq, 21.9 mmol). The reaction mixture was heated at 50° C. for 2 h. The reaction mixture was cooled to rt and the volatiles were removed in vacuo. The residue was redissolved in MTBE (25 mL) and transferred into a separating funnel. The aqueous layer was extracted with MTBE (3×25 mL). The combined organic layers were collected, dried (Na₂SO₄), filtered and concentrated in vacuo affording the sub-title compound (IM-2) which was taken forward crude without purification. ¹H NMR (400 MHz, CDCl3) δ 2.57-2.45 (m, 2H), 2.44-2.33 (m, 1H), 2.20-2.09 (m, 1H), 2.09-1.94 (m, 2H), 1.46 (s, 3H).

Step 2: rac-(1S,2S)-2-Hydroxy-1-methylcyclopentane-1-carbonitrile (IM-4). To a stirred solution of the product from step 1 above (IM-2) (1.058 g, 82% Wt, 1 Eq, 7.044 mmol) in MeOH (30 mL) at 0° C. was added NaBH₄ (144.8 mg, 98% Wt, 0.5325 Eq, 3.751 mmol) in small portions. The reaction mixture was stirred at rt for 1 h. Distilled water (10 mL) was added and the volatiles were removed in vacuo. The aqueous layer was extracted with MTBE (3×25 mL). The combined organic layers were collected, dried (Na₂SO₄), filtered and concentrated in vacuo. The crude product was purified by chromatography on silica gel (40 g cartridge, 0-100% MTBE/iso-hexane) to afford rac-(1S,2R)-2-hydroxy-1-methylcyclopentane-1-carbonitrile (IM-3) (444.5 mg, 2.5 mmol, 35%, 70% Purity) as a colourless oil. ¹H NMR (400 MHz, CDCl3) δ 4.33 (t, J=5.4 Hz, 1H), 2.26-2.15 (m, 1H), 2.15-2.07 (m, 1H), 1.91-1.76 (m, 3H), 1.76-1.65 (m, 2H), 1.39 (s, 3H). Further elution also provided the sub-title compound (IM-4) (704.3 mg, 3.3 mmol, 46%, 58% Purity) as a colourless oil. ¹H NMR (400 MHz, CDCl3) δ 3.90 (t, J=5.9 Hz, 1H), 2.33-2.24 (m, 1H), 2.14-2.05 (m, 1H), 2.01-1.90 (m, 1H), 1.85-1.64 (m, 4H), 1.36 (s, 3H).

Step 3: rac-(1R,2S)-2-Hydroxy-1-methylcyclopentane-1-carboxamide (IM-5). To a stirred solution of the product from step 2 above (IM-4) (395.5 mg, 58% Wt, 1 Eq, 1.833 mmol) in water (0.3 mL) at 0° C. was added sulfuric acid (5.5 g, 3.0 mL, 31 Eq, 56 mmol). The reaction mixture was stirred at rt for 1 h. The reaction mixture was cooled to 0° C., neutralized with 2 M NaOH and extracted with EtOAc (3×5 mL). The combined organic layers were collected, dried (Na₂SO₄), filtered and concentrated in vacuo. The crude product was purified by chromatography on silica gel (12 g cartridge, 0-100% EtOAc/isohexane and then 0-100% (0.7 M ammonia/MeOH)/DCM) to afford the sub-title compound (IM-5) (152.4 mg, 0.53 mmol, 29%, 50% Purity) as a colourless oil, partially contaminated with unreacted starting material (IM-4). ¹H NMR (400 MHz, MeOD) δ 4.42 (t, J=6.9 Hz, 1H), 2.28-2.13 (m, 1H), 2.09-1.97 (m, 1H), 1.82-1.61 (m, 4H), 1.45 (s, 3H). 3× exchangeable protons not observed. Some additional starting material (IM-4) (69.4 mg, 0.25 mmol, 14%, 45% Purity) was also recovered as a colourless oil.

Step 4: rac-(3aR,6aS)-3a-Methylhexahydro-2H-cyclopenta[d]oxazol-2-one (IM-6). To a stirred solution of the product from step 3 above (IM-5) in dry MeCN (8.0 mL) at rt was added phenyliodine(III) bis(trifluoroacetate) (456 mg, 1.99 Eq, 1.06 mmol). The reaction mixture was stirred at rt for 3 d. The volatiles were removed in vacuo. The crude product was purified by chromatography on silica gel (12 g cartridge, 0-100% EtOAc/isohexane) to afford the sub-title compound (IM-6) (128.0 mg, 0.45 mmol, 85%, 50% Purity) as a yellow oil. ¹H NMR (400 MHz, MeOD) δ 4.57 (dd, J=5.1, 1.2 Hz, 1H), 2.23-2.14 (m, 1H), 2.10-1.90 (m, 1H), 1.90-1.66 (m, 4H), 1.42 (s, 3H). 1× exchangeable NH proton not observed.

Step 5: rac-(1S,2R)-2-Amino-2-methylcyclopentan-1-ol, HCl (IM-7). To a stirred solution of the product from step 4 above (IM-6) (128.0 mg, 50% Wt, 1 Eq, 453.4 μmol) in EtOH (1.0 mL) at rt was added KOH (106.8 mg, 4.198 Eq, 1.903 mmol) in water (1.0 mL). The reaction mixture was heated at 80° C. overnight. The reaction mixture was cooled to rt, diluted with EtOAc (2 mL) and neutralised with 1 M HCl (2 mL). The aqueous layer was extracted with EtOAc (3×2 mL). The combined organic layers were collected, dried (Na₂SO₄), filtered and concentrated in vacuo to afford intermediate (IM-4) (44.6 mg, 356 μmol, 78.6%), carried through from previous steps, as a yellow oil. The aqueous layer was concentrated in vacuo to afford the sub-title compound (IM-7) (125.2 mg, 0.41 mmol, 91%, 50% Purity) as a white solid. ¹H NMR (400 MHz, MeOD) δ 3.87 (dd, J=6.0, 4.6 Hz, 1H), 2.11-2.01 (m, 1H), 2.00-1.83 (m, 2H), 1.83-1.65 (m, 3H), 1.33 (s, 3H). Exchangeable protons not observed.

Step 6: rac-(1S,2R)-2-(((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)amino)-2-methylcyclopentan-1-ol (IM-8). A solution of intermediate (Y-1) (51.4 mg, 94% Wt, 1 Eq, 104 μmol), the product from step 5 above (IM-7) (125 mg, 30 Wt %, 3.15 Eq, 0.326 mmol) and DIPEA (74.2 mg, 100 μL, 5.54 Eq, 574 μmol) in CHCl₃ (2.0 mL) was stirred 1 h at rt. NaBH(OAc)₃ (71.6 mg, 97% Wt, 3.16 Eq, 328 μmol) was added and the reaction mixture was stirred at rt for 2 h. NaBH(OAc)₃ (70.2 mg, 97% Wt, 3.10 Eq, 321 μmol) was added and the resulting mixture was stirred at rt overnight. The reaction mixture was diluted with EtOAc (5 mL), washed with sat. NaHCO₃ (5 mL) and brine (5 mL), dried (Na₂SO₄), filtered and concentrated in vacuo. The crude product was purified by chromatography on silica gel (4 g cartridge, 0-10% MeOH/DCM) to afford the title compound (IM-8) (7.8 mg, 13 μmol, 13%, 97% Purity) as an off-white solid. m/z 566.2 (M+H)⁺ (ES+). ¹H NMR (400 MHz, MeOD) δ 8.38 (s, 1H), 8.21 (dt, J=7.9, 1.4 Hz, 1H), 8.08-8.02 (m, 2H), 7.82-7.74 (m, 2H), 7.61-7.51 (m, 2H), 7.49-7.42 (m, 2H), 4.06 (d, J=12.4 Hz, 1H), 4.02 (d, J=12.4 Hz, 1H), 3.92 (d, J=4.9 Hz, 1H), 3.25 (s, 3H), 2.09-1.98 (m, 1H), 1.95-1.75 (m, 3H), 1.75-1.61 (m, 2H), 1.26 (s, 3H). 2× exchangeable protons not observed.

### Example 357: 2-(4′-Fluoro-2′-(4-methyl-4H-1,2,4-triazol-3-yl)-[1,1′-biphenyl]-3-yl)-7-(trifluoromethyl)benzo[d]oxazole-5-carboxylic Acid (DB-1)

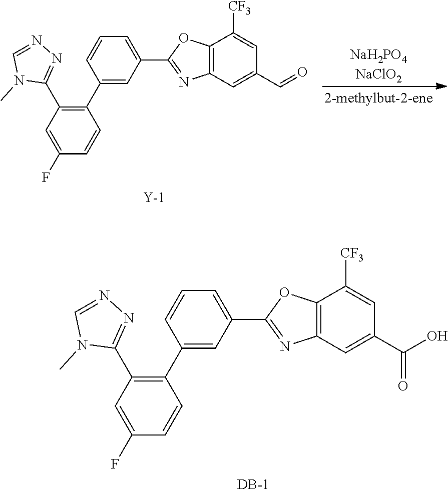

To a stirred solution of intermediate (Y-1) (26.1 mg, 97% Wt, 1 Eq, 54.3 μmol) and 2-methylbut-2-ene (17 mg, 25 μL, 95% Wt, 4.2 Eq, 0.23 mmol) in t-BuOH (1.5 mL) at rt was added NaH₂PO₄ (66.6 mg, 48 μL, 10.2 Eq, 555 μmol) and NaClO₂ (24.6 mg, 80% Wt, 4.01 Eq, 218 μmol) in water (0.50 mL). The reaction mixture was stirred at rt overnight. The reaction mixture was basified with 2 M NaOH and extracted with isohexane (3×5 mL). The aqueous layers was acidified by 1 M HCl to pH -3 and extracted with EtOAc (3×5 mL). The combined organic layers were collected, dried (Na₂SO₄), filtered and concentrated in vacuo to afford impure product. The crude was purified by preparative HPLC (Waters, Acidic (0.1% formic acid), Acidic, Waters X-Select CSH C18 ODB, 5 μm, 30×100 mm column, 35-100% MeCN in water) to afford the title compound (7.6 mg, 16 μmol, 29%, 99% Purity) as an off-white solid. m/z 483.0 (M+H)⁺ (ES+). ¹H NMR (400 MHz, DMSO) δ 13.65 (br s, 1H), 8.59 (d, J=1.5 Hz, 1H), 8.42 (s, 1H), 8.25 (s, 1H), 8.16 (d, J=7.9 Hz, 1H), 7.94 (d, J=1.9 Hz, 1H), 7.78 (dd, J=8.6, 5.6 Hz, 1H), 7.65-7.52 (m, 3H), 7.40 (dt, J=7.8, 1.4 Hz, 1H), 3.21 (s, 3H).

### Example 358: 5-(((1S,4S)-2-Oxa-5-azabicyclo[2.2.1]heptan-5-yl)methyl)-2-(4′-fluoro-2′-(4-methyl-4H-1,2,4-triazol-3-yl)-[1,1′-biphenyl]-3-yl)-7-(trifluoromethyl)benzo[d]oxazole (IN-2)

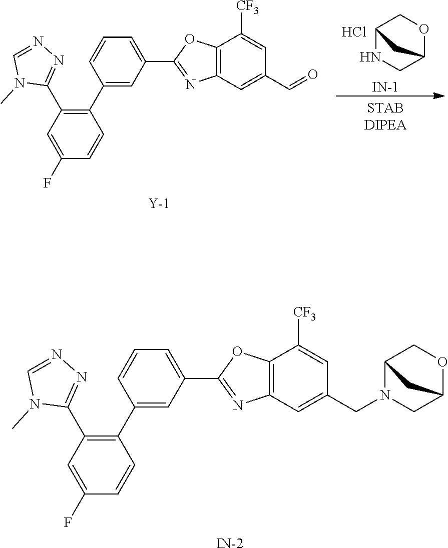

A solution of intermediate (Y-1) (26.7 mg, 93% Wt, 1 Eq, 53.2 μmol), (1S,4S)-2-oxa-5-azabicyclo[2.2.1]heptane, HCl (IN-1) (14.4 mg, 2 Eq, 106 μmol) and DIPEA (20.6 mg, 27.8 μL, 3 Eq, 160 μmol) in DCM (3 mL) was stirred at rt for 30 min. NaBH(OAc)₃ (33.9 mg, 3 Eq, 160 μmol) was added and the reaction mixture was stirred at rt overnight. The reaction mixture was diluted with sat. NaHCO₃ (5 mL) and transferred into a separating funnel. The layer was extracted with DCM (3×10 mL). The combined extracted layers were collected, dried (Na₂SO₄), filtered and concentrated in vacuo. The crude product was purified by chromatography on silica gel (4 g cartridge, 0-10% MeOH/DCM) to afford the title compound (IN-2) (12.93 mg, 23 μmol, 43%, 98% Purity) as an off-white solid. m/z 550.4 (M+H)⁺ (ES+). ¹H NMR (400 MHz, DMSO) δ 8.41 (s, 1H), 8.13 (dt, J=7.9, 1.4 Hz, 1H), 8.07 (s, 1H), 7.90 (t, J=1.8 Hz, 1H), 7.80-7.73 (m, 2H), 7.64-7.53 (m, 3H), 7.38 (dt, J=7.8, 1.4 Hz, 1H), 4.37 (s, 1H), 3.94 (dd, J=17.3, 6.9 Hz, 3H), 3.55 (dd, J=7.5, 1.8 Hz, 1H), 3.50 (s, 1H), 3.20 (s, 3H), 2.77 (dd, J=9.9, 1.8 Hz, 1H), 2.46 (d, J=9.9 Hz, 1H), 1.85 (dd, J=9.7, 2.1 Hz, 1H), 1.62 (d, J=9.5 Hz, 1H).

### Example 359: rac-N-(Cyclobutylmethyl)-1-(7-cyclopropyl-2-(4′-fluoro-2′-(4-methyl-4H-1,2,4-triazol-3-yl)-[1,1′-biphenyl]-3-yl)benzo[d]oxazol-5-yl)-2,2,2-trifluoroethan-1-amine (IO-4)

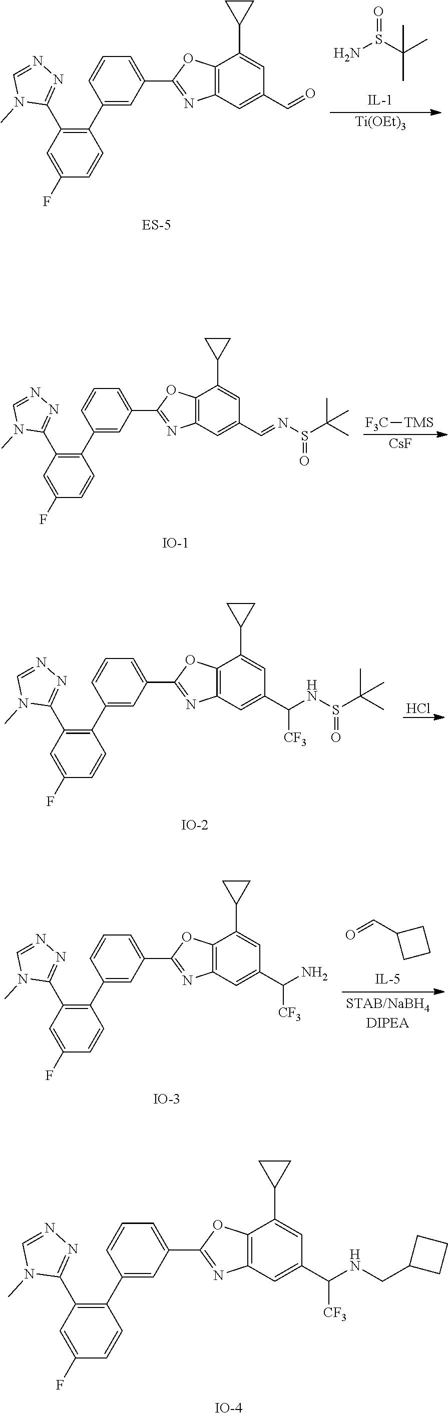

Step 1: (E)-N-((7-Cyclopropyl-2-(4′-fluoro-2′-(4-methyl-4H-1,2,4-triazol-3-yl)-[1,1′-biphenyl]-3-yl)benzo[d]oxazol-5-yl)methylene)-2-methylpropane-2-sulfinamide (IO-1). To a stirred combined solution of two batches of intermediate (ES-5) (161.8 mg, 70% Wt, 1 Eq, 258.3 μmol) and (36.1 mg, 75% Wt, 0.239 Eq, 61.8 μmol) and 2-Methyl-2-propanesulfinamide (IL-1) (61.99 mg, 1.98 Eq, 511.5 μmol) in dry THF (3.0 mL) under a nitrogen atmosphere at rt was added Ti(OEt)₃ (219.2 mg, 200.0 μL, 3.72 Eq, 960.9 μmol). The reaction mixture was heated at 60° C. overnight. The reaction mixture was cooled to rt, sat. Rochelle salt solution (5 mL) was added and the resulting mixture was stirred at rt for 15 min. The mixture was then extracted with EtOAc (3×5 mL). The combined organic layers were collected, dried (Na₂SO₄), filtered and concentrated in vacuo. The crude product was purified by chromatography on silica gel (12 g cartridge, 0-10% MeOH/DCM) to afford the sub-title compound (IO-1) (184.8 mg, 0.27 mmol, 85%, 80% Purity) as a yellow solid. m/z 542.1 (M+H)⁺ (ES+). ¹H NMR (400 MHz, DMSO) δ 8.64 (s, 1H), 8.43 (s, 1H), 8.19-8.14 (m, 2H), 7.94 (s, 1H), 7.79 (dd, J=8.5, 5.7 Hz, 1H), 7.71 (d, J=1.4 Hz, 1H), 7.66-7.52 (m, 3H), 7.38 (d, J=7.8 Hz, 1H), 3.17 (s, 3H), 2.42-2.34 (m, 1H), 1.21 (s, 9H), 1.18-1.13 (m, 2H), 1.13-1.10 (m, 2H).

Step 2: rac-N-(1-(7-Cyclopropyl-2-(4′-fluoro-2′-(4-methyl-4H-1,2,4-triazol-3-yl)-[1,1′-biphenyl]-3-yl)benzo[d]oxazol-5-yl)-2,2,2-trifluoroethyl)-2-methylpropane-2-sulfinamide (IO-2). To a stirred solution of the product from step 1 above (IO-1) (184.8 mg, 80% Wt, 1 Eq, 272.9 μmol) in dry DME (3.0 mL) under a nitrogen atmosphere at 0° C. was added trifluoromethyltrimethylsilane (285 mg, 300 μL, 7.34 Eq, 2.00 mmol). The reaction mixture was stirred at 0° C. for 30 min and then at rt for 2 h. The reaction mixture was diluted with brine (5 mL), extracted with EtOAc (3×5 mL). The combined organic layers were collected, dried (Na₂SO₄), filtered and concentrated in vacuo. The crude product was purified by chromatography on silica gel (12 g cartridge, 0-10% MeOH/DCM) to afford the sub-title compound (IO-2) (36.1 mg, 41 μmol, 15%, 70% Purity) as a yellow solid. m/z 612.2 (M+H)⁺ (ES+). ¹H NMR (400 MHz, MeOD) δ 8.38 (s, 1H), 8.20 (dt, J=7.7, 1.3 Hz, 1H), 8.03 (dt, J=3.4, 1.8 Hz, 1H), 7.78 (dd, J=8.6, 5.4 Hz, 1H), 7.60-7.51 (m, 2H), 7.51-7.37 (m, 4H), 5.20-5.12 (m, 1H), 3.18 (s, 3H), 2.40-2.31 (m, 1H), 1.25 (s, 6H), 1.21 (s, 3H), 1.19-1.16 (m, 2H), 1.10-1.00 (m, 2H). 1× exchangeable NH proton not observed.

Step 3: rac-1-(7-Cyclopropyl-2-(4′-fluoro-2′-(4-methyl-4H-1,2,4-triazol-3-yl)-[1,1′-biphenyl]-3-yl)benzo[d]oxazol-5-yl)-2,2,2-trifluoroethan-1-amine, HCl (IO-3). To a stirred solution of the product from step 2 above (IO-2) (36.1 mg, 70% Wt, 1 Eq, 41.3 μmol) in MeOH (2.0 mL) at rt was added HCl in 1,4-dioxane (7.53 mg, 51.6 μL, 4 molar, 5 Eq, 207 μmol). The reaction mixture was stirred at rt overnight. The volatiles were removed in vacuo to afford the sub-title compound (IO-3) (26.7 mg, 39 μmol, 95%, 80% Purity) as a yellow solid. m/z 508.2 (M+H)⁺ (ES+).

Step 4: rac-N-(Cyclobutylmethyl)-1-(7-cyclopropyl-2-(4′-fluoro-2′-(4-methyl-4H-1,2,4-triazol-3-yl)-[1,1′-biphenyl]-3-yl)benzo[d]oxazol-5-yl)-2,2,2-trifluoroethan-1-amine (IO-4). A solution of the product from step 3 above (IO-3) (22.5 mg, 1 Eq, 41.4 μmol), cyclobutanaldehyde (IL-5) (14 mg, 15 μL, 4.0 Eq, 0.17 mmol) and DIPEA (37 mg, 50 μL, 6.9 Eq, 0.29 mmol) in CHCl₃ (2.0 mL) was stirred 1 h at rt. NaBH(OAc)₃ (45.2 mg, 97% Wt, 5 Eq, 207 μmol) was added and the reaction mixture was stirred at rt for 3 h. Cyclobutanaldehyde (IL-5) (14 mg, 15 μL, 4.0 Eq, 0.17 mmol) was added and the reaction mixture was heated at 45° C. overnight. The reaction mixture was cooled to rt. NaBH₄ (1.60 mg, 98% Wt, 1 Eq, 41.4 μmol) was added and the resulting mixture was stirred at rt overnight. The reaction mixture was diluted with EtOAc (5 mL), washed with sat. NaHCO₃ (5 mL) and brine (5 mL), dried (Na₂SO₄), filtered and concentrated in vacuo. The crude product was purified by chromatography on silica gel (4 g cartridge, 0-10% MeOH/DCM) to afford impure product. The crude was purified by preparative HPLC (Waters, Acidic (0.1% formic acid), Acidic, Waters X-Select CSH C18 ODB, 5 μm, 30×100 mm column, 55-100 MeCN in water) to afford the title compound (IO-4) (3.4 mg, 5.8 μmol, 14%, 98% Purity) as an off-white solid. m/z 576.1 (M+H)⁺ (ES+). ¹H NMR (400 MHz, MeOD) δ 8.38 (s, 1H), 8.21 (dt, J=7.8, 1.4 Hz, 1H), 8.03 (t, J=1.8 Hz, 1H), 7.79 (dd, J=8.6, 5.4 Hz, 1H), 7.64-7.51 (m, 3H), 7.50-7.42 (m, 2H), 7.17 (d, J=1.5 Hz, 1H), 4.35 (q, J=7.7 Hz, 1H), 3.19 (s, 3H), 2.63-2.55 (m, 1H), 2.55-2.41 (m, 2H), 2.40-2.30 (m, 1H), 2.10-1.98 (m, 2H), 1.95-1.84 (m, 1H), 1.84-1.74 (m, 1H), 1.68-1.57 (m, 2H), 1.22-1.13 (m, 2H), 1.04 (dt, J=6.8, 4.4 Hz, 2H). 1× exchangeable NH proton not observed.

### Example 360: (1S,2R)-2-(((6-Fluoro-2-(4′-fluoro-2′-(4-methyl-4H-1,2,4-triazol-3-yl)-[1,1′-biphenyl]-3-yl)benzo[d]oxazol-5-yl)methyl)amino)cyclopentan-1-ol (IP-7)

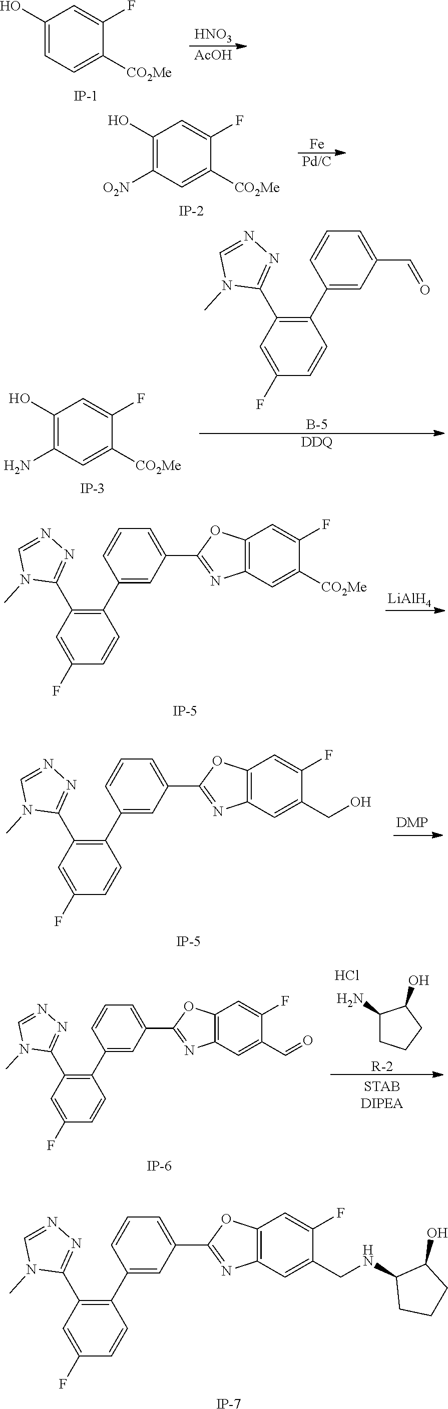

Step 1: Methyl 2-fluoro-4-hydroxy-5-nitrobenzoate (IP-2). To a mixture of methyl 2-fluoro-4-hydroxybenzoate (IP-1) (500 mg, 1 Eq, 2.94 mmol) suspended in AcOH (5.29 g, 5.05 mL, 30 Eq, 88.2 mmol) was added nitric acid (397 mg, 0.28 mL, 70% Wt, 1.5 Eq, 4.41 mmol) in AcOH (2 mL) slowly at 0° C. The resulting suspension was stirred at this temperature for 30 min and then slowly warm it to rt and stirred for 12 h. The reaction mixture was diluted with distilled water (25 mL) and transferred into a separating funnel. The layer was extracted with DCM (3×25 mL). The combined extracted layers were collected, dried (Na₂SO₄), filtered and concentrated in vacuo. The crude product was purified by chromatography on silica gel (40 g cartridge, 0-50% EtOAc/isohexane) to afford the sub-title compound (IP-2) (319 mg, 1.5 mmol, 50%, 99% Purity) and regioisomer methyl 2-fluoro-4-hydroxy-3-nitrobenzoate (120 mg, 0.55 mmol, 19%, 98% Purity) as clear white powders. m/z 214.00 (M−H)⁻ (ES−). ¹H NMR (400 MHz, DMSO) δ=12.5 (brs, 1H), 8.45 (d, J=7.8 Hz, 1H), 6.98 (d, J=12.2 Hz, 1H), 3.84 (s, 3H).

Step 2: Methyl 5-amino-2-fluoro-4-hydroxybenzoate (IP-3). To a stirred solution of the product from step 1 above (IP-2) (300 mg, 1 Eq, 1.39 mmol) in dry EtOH (3 mL) under a nitrogen atmosphere at 25° C. was added Pd/C (297 mg, 5% Wt, 0.1 Eq, 139 μmol). The reaction mixture was purged with H₂ (5 bar) and stirred for 3 h. The crude mass was filtered through celite pad and concentrated in vacuo. The crude product was purified by chromatography on silica gel (12 g cartridge, 0-10% EtOAc/isohexane) to afford the sub-title compound (IP-3) (165 mg, 0.87 mmol, 63%, 98% Purity) as a clear white powder. m/z 186.1 (M+H)⁺ (ES+).

Step 3: Methyl 6-fluoro-2-(4′-fluoro-2′-(4-methyl-4H-1,2,4-triazol-3-yl)-[1,1′-biphenyl]-3-yl)benzo[d]oxazole-5-carboxylate (IP-4). To a solution of intermediate (B-5) (290 mg, 64% Wt, 1.0 Eq, 659 μmol) in EtOH (3.00 mL) was added the product from step 2 above (IP-3) (165 mg, 74% Wt, 1.0 Eq, 659 μmol) in a vial. The resulting mixture was stirred at 80° C. for over 12 h. The reaction mixture was dried and dissolved in DCM (2 mL) then DDQ (165 mg, 1.1 Eq, 725 μmol) was added and continue the stirring at rt for another 2 h. The reaction mixture was diluted with sat. NaHCO₃ (10 mL) and transferred into a separating funnel. The layer was extracted with DCM (3×10 mL). The combined extracted layers were collected, dried (Na₂SO₄), filtered and concentrated in vacuo. The crude product was purified by chromatography on silica gel (24 g cartridge, 0-10% MeOH/DCM) to afford the sub-title compound (IP-4) (190 mg, 0.41 mmol, 63%, 97% Purity) as a thick brown gum. m/z 447.1 (M+H) (ES+).

Step 4: (6-Fluoro-2-(4′-fluoro-2′-(4-methyl-4H-1,2,4-triazol-3-yl)-[1,1′-biphenyl]-3-yl)benzo[d]oxazol-5-yl)methanol (IP-5). To a solution of the product from step 3 above (IP-4) (150 mg, 1 Eq, 336 μmol) in THF (2 mL) was added LiAlH₄ (15.3 mg, 403 μL, 1 molar, 1.2 Eq, 403 μmol) dropwise at 0° C. This mixture was stirred 2 h at this temperature. The reaction mixture was diluted with distilled water (10 mL) and acidified with 2 mL 1M HCl and transferred into a separating funnel. The layer was extracted with DCM (3×10 mL). The combined extracted layers were collected, dried (Na₂SO₄), filtered and concentrated in vacuo affording the sub-title compound (IP-5) (140 mg, 0.31 mmol, 94%, 95% Purity), which was taken forward crude without purification. m/z 419.1 (M+H). (ES+).

Step 5: 6-Fluoro-2-(4′-fluoro-2′-(4-methyl-4H-1,2,4-triazol-3-yl)-[1,1′-biphenyl]-3-yl)benzo[d]oxazole-5-carbaldehyde (IP-6). To a stirred solution of the product from step 4 above (IP-5) (140 mg, 94% Wt, 1 Eq, 315 μmol) in dry DCM (2 mL) under a nitrogen atmosphere at 25° C. was added DMP (267 mg, 2 Eq, 629 μmol). The reaction mixture was stirred for 3 h. The reaction mixture was diluted with DCM (10 mL) and washed with sat. aq. NaHCO₃ (10 mL) and brine. Organics were separated, dried (MgSO₄), filtered and evaporated in vacuo. The crude product was purified by chromatography on silica gel (12 g cartridge, 0-10% (0.7 M ammonia/MeOH)/DCM) to afford the sub-title compound (IP-6) (125 mg, 0.29 mmol, 93%, 97% Purity) as a pale-yellow powder. m/z 417.3 (M+H)⁺ (ES+).

Step 6: (1S,2R)-2-(((6-Fluoro-2-(4′-fluoro-2′-(4-methyl-4H-1,2,4-triazol-3-yl)-[1,1′-biphenyl]-3-yl)benzo[d]oxazol-5-yl)methyl)amino)cyclopentan-1-ol (IP-7). A solution of the product from step 5 above (IP-6) (40.0 mg, 1 Eq, 96.1 μmol), (1S,2R)-2-(chloro-15-azaneyl)cyclopentan-1-ol (R-2) (26.4 mg, 2 Eq, 192 μmol) and DIPEA (62.1 mg, 83.7 μL, 5 Eq, 480 μmol) in CHCl₃ (1 mL) was stirred at 45° C. for 5 h. NaBH(OAc)₃ (102 mg, 5 Eq, 480 μmol) was added and the reaction mixture was stirred at 25° C. for another 48 h. The reaction mixture was diluted with Sat. NaHCO₃ (10 mL) and transferred into a separating funnel. The layer was extracted with DCM (3×10 mL). The combined extracted layers were collected, dried (Na₂SO₄), filtered and concentrated in vacuo. The crude product was purified by chromatography on silica gel (4 g cartridge, 0-10% MeOH/DCM) to afford the title compound (IP-7) (38.0 mg, 75 μmol, 78%, 99% Purity) as a clear white powder. m/z 502.1 (M+H)⁺ (ES+). ¹H NMR (400 MHz, MeOD) δ=8.40 (s, 1H), 8.20 (dt, J=7.9, 1.4 Hz, 1H), 8.07 (t, J=1.8 Hz, 1H), 7.85-7.77 (m, 2H), 7.56 (ddt, J=13.9, 8.1, 3.5 Hz, 3H), 7.49 (dd, J=8.8, 2.7 Hz, 1H), 7.43 (ddd, J=7.8, 1.9, 1.2 Hz, 1H), 4.17 (td, J=4.4, 2.4 Hz, 1H), 4.08-3.93 (m, 2H), 3.21 (s, 3H), 3.02 (td, J=8.2, 4.1 Hz, 1H), 1.98-1.67 (m, 4H), 1.66-1.49 (m, 2H). Exchangeable NH & OH not observed.

### Example 361: (1S,2R)-2-(((4-Fluoro-2-(4′-fluoro-2′-(4-methyl-4H-1,2,4-triazol-3-yl)-[1,1′-biphenyl]-3-yl)-7-methylbenzo[d]oxazol-5-yl)methyl)amino)cyclopentan-1-ol (IQ-8)

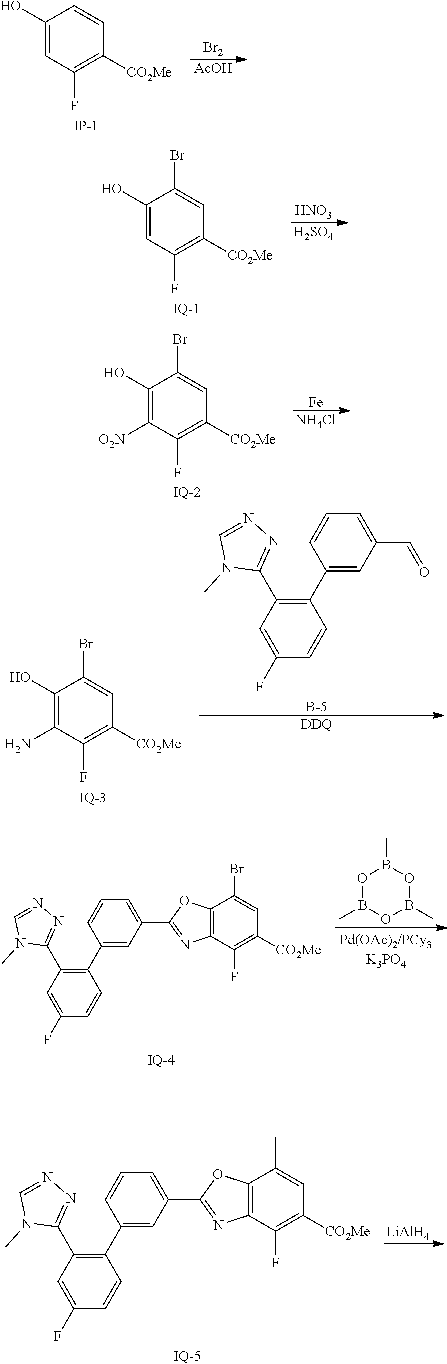

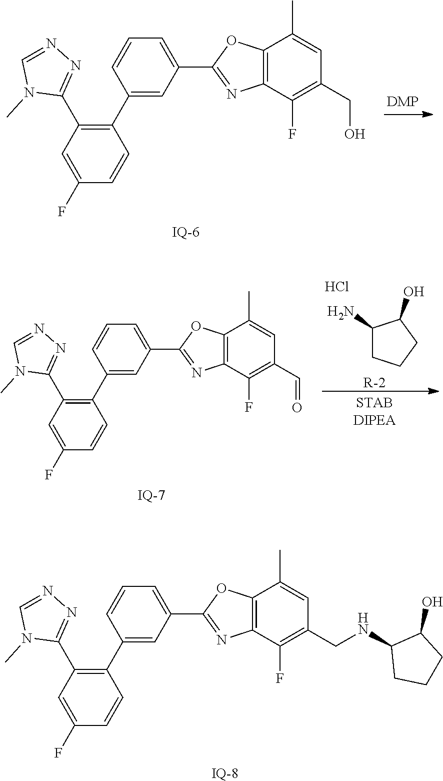

Step 1: Methyl 5-bromo-2-fluoro-4-hydroxybenzoate (IQ-1). To a stirred solution of methyl 2-fluoro-4-hydroxybenzoate (IP-1) (1 g, 1 Eq, 6 mmol) in AcOH (7 g, 7 mL, 20 Eq, 0.1 mol) under a N₂ atmosphere at 25° C. was slowly dropwise added bromine (0.9 g, 0.3 mL, 1 Eq, 6 mmol) in AcOH (1 mL). The reaction mixture was stirred for 12 h. The reaction mixture was diluted with distilled water (50 mL) and transferred into a separating funnel. The layer was extracted with EtOAc (3×25 mL). The combined extracted layers were collected, dried (Na₂SO₄), filtered and concentrated in vacuo. The crude product was purified by chromatography on silica gel (40 g cartridge, 50-100% EtOAc/isohexane) to afford the sub-title compound (IQ-1) (700 mg, 2.7 mmol, 46%, 95% Purity) as a clear white solid. ¹H NMR (400 MHz, CDCl3) δ=8.12 (d, J=7.4 Hz, 1H), 6.81 (d, J=11.4 Hz, 1H), 6.01 (d, J=1.5 Hz, 1H), 3.91 (s, 3H).

Step 2: Methyl 5-bromo-2-fluoro-4-hydroxy-3-nitrobenzoate (IQ-2). To a mixture of the product from step 1 above (IQ-1) (560 mg, 1 Eq, 2.25 mmol) suspended in conc. sulfuric acid (7.72 g, 4.19 mL, 35 Eq, 78.7 mmol) was added nitric acid (405 mg, 0.29 mL, 70% Wt, 2.0 Eq, 4.50 mmol) slowly at 0° C. The resulting suspension was stirred at this temperature for 15 min. The reaction mixture was diluted with ice water (100 mL) and transferred into a separating funnel. The layer was extracted with DCM (3×25 mL). The combined extracted layers were collected, dried (Na₂SO₄), filtered and concentrated in vacuo to afford the sub-title compound (IQ-2) (410 mg, 1.2 mmol, 52%, 84% Purity) as a pale-yellow solid. m/z 295.1 (M+H)⁺ (ES+).

Step 3: Methyl 3-amino-5-bromo-2-fluoro-4-hydroxybenzoate (IQ-3). To a stirred solution of the product from step 2 above (IQ-2) (800 mg, 55% Wt, 1 Eq, 1.50 mmol) in dry EtOH (10 mL) under a nitrogen atmosphere at 80° C. was added iron (334 mg, 4 Eq, 5.99 mmol) and NH₄Cl (1.7 g) in H₂O(3 mL). The reaction mixture was stirred for 12 h at 80° C. After completion of the reaction the crude was filtered through the celite pad and washed thoroughly with MeOH. The reaction mixture was concentrated in vacuo. The crude product was purified by chromatography on silica gel (12 g cartridge, 15-75% EtOAc/isohexane) to afford the sub-title compound (IQ-3) (135 mg, 0.40 mmol, 27%, 79% Purity) as a light brown solid. m/z 266.0/264.0 (M+H)⁺ (ES+).

Step 4: Methyl 7-bromo-4-fluoro-2-(4′-fluoro-2′-(4-methyl-4H-1,2,4-triazol-3-yl)-[1,1′-biphenyl]-3-yl)benzo[d]oxazole-5-carboxylate (IQ-4). To a solution of intermediate (B-5) (166 mg, 64% Wt, 1.0 Eq, 378 μmol) in EtOH (3.00 mL) was added the product from step 3 above (IQ-3) (135 mg, 74% Wt, 1.0 Eq, 378 μmol) in a vial. The resulting mixture was stirred at 80° C. for over 5 h. The reaction mixture was dried and dissolved in DCM (2 mL) then DDQ (85.9 mg, 1 Eq, 378 μmol) was added and continue the stirring at rt for another 2 h. The reaction mixture was diluted with sat. NaHCO₃ (10 mL) and transferred into a separating funnel. The layer was extracted with DCM (3×10 mL). The combined extracted layers were collected, dried (Na₂SO₄), filtered and concentrated in vacuo. The crude product was purified by chromatography on silica gel (24 g cartridge, 0-10% MeOH/DCM) to afford the sub-title compound (IQ-4) (140 mg, 0.25 mmol, 66%, 94% Purity) as a thick brown gum. m/z 526.3 (M+H)⁺ (ES+).

Step 5: Methyl 4-fluoro-2-(4′-fluoro-2′-(4-methyl-4H-1,2,4-triazol-3-yl)-[1,1′-biphenyl]-3-yl)-7-methylbenzo[d]oxazole-5-carboxylate (IQ-5). To a solution of the product from step 4 above (IQ-4) (120 mg, 1 Eq, 228 μmol) and tricyclohexylphosphane (9.61 mg, 0.15 Eq, 34.3 μmol) in toluene (0.7 mL) and water (0.3 mL) was added potassium phosphate (145 mg, 3 Eq, 685 μmol). The reaction mixture was degassed (evacuated and backfilled, N₂×3) and 2,4,6-trimethyl-1,3,5,2,4,6-trioxatriborinane (115 mg, 4 Eq, 914 μmol) and Pd(OAc)₂ (7.69 mg, 0.15 Eq, 34.3 μmol) was added. The reaction was heated to 110° C. for 1.5 h. in a microwave. The reaction mixture was diluted with distilled water (5 mL) and transferred into a separating funnel. The layer was extracted with DCM (3×5 mL). The combined extracted layers were collected, dried (Na₂SO₄), filtered and concentrated in vacuo. The crude product was purified by chromatography on silica gel (4 g cartridge, 0-10% MeOH/DCM) to afford the sub-title compound (IQ-5) (42.0 mg, 69 μmol, 30%, 76% Purity) as alight brown solid. m/z 461.1 (M+H)⁺ (ES+).

Step 6: (4-Fluoro-2-(4′-fluoro-2′-(4-methyl-4H-1,2,4-triazol-3-yl)-[1,1′-biphenyl]-3-yl)-7-methylbenzo[d]oxazol-5-yl)methanol (IQ-6). To a solution of the product from step 5 above (IQ-5) (40.0 mg, 1 Eq, 86.9 μmol) in THF (2 mL) was added LiAlH₄ (3.96 mg, 104 μL, 1 molar, 1.2 Eq, 104 μmol) dropwise at 0° C. This mixture was stirred 2 h at this temperature. The reaction mixture was diluted with distilled water (10 mL) and acidified with 2 mL 1M HCl and transferred into a separating funnel. The layer was extracted with DCM (3×10 mL). The combined extracted layers were collected, dried (Na₂SO₄), filtered and concentrated in vacuo affording the sub-title compound (IQ-6) (27 mg, 47 μmol, 55%, 76% Purity) which was carried forward without further purification. m/z 431.2 (M+H)⁺ (ES+).

Step 7: 4-Fluoro-2-(4′-fluoro-2′-(4-methyl-4H-1,2,4-triazol-3-yl)-[1,1′-biphenyl]-3-yl)-7-methylbenzo[d]oxazole-5-carbaldehyde (IQ-7). To a stirred solution of the product from step 6 above (IQ-6) (30.0 mg, 75% Wt, 1 Eq, 52.0 μmol) in dry DCM (2 mL) under a nitrogen atmosphere at 25° C. was added DMP (44.1 mg, 2 Eq, 104 μmol). The reaction mixture was stirred for 3 h. The reaction mixture was diluted with DCM (10 mL) and washed with sat. aq. NaHCO₃ (10 mL) and brine. Organics were separated, dried (MgSO₄), filtered and evaporated in vacuo. The crude product was purified by chromatography on silica gel (4 g cartridge, 0-10% (0.7 M ammonia/MeOH)/DCM) to afford the sub-title compound (IQ-7) (26.0 mg, 45 μmol, 87%, 75% Purity) as a pale-yellow solid. m/z 431.1 (M+H)⁺ (ES+).

Step 8: (1S,2R)-2-(((4-Fluoro-2-(4′-fluoro-2′-(4-methyl-4H-1,2,4-triazol-3-yl)-[1,1′-biphenyl]-3-yl)-7-methylbenzo[d]oxazol-5-yl)methyl)amino)cyclopentan-1-ol (IQ-8). A solution of the product from step 7 above (IQ-7) (26.0 mg, 75% Wt, 1 Eq, 45.3 μmol), (1S,2R)-2-(chloro-l5-azaneyl)cyclopentan-1-ol (R-2) (12.5 mg, 2 Eq, 90.6 μmol) and DIPEA (29.3 mg, 39.5 μL, 5 Eq, 227 μmol) in CHCl₃ (1 mL) was stirred at 40° C. for 12 h. NaBH(OAc)₃ (48.0 mg, 5 Eq, 227 μmol) was added and the reaction mixture was stirred at 40° C. for another 2 h. The reaction mixture was diluted with sat. NaHCO₃ (10 mL) and transferred into a separating funnel. The layer was extracted with DCM (3×10 mL). The combined extracted layers were collected, dried (Na₂SO₄), filtered and concentrated in vacuo. The crude product was purified by chromatography on silica gel (4 g cartridge, 0-10% MeOH/DCM) to afford the title compound (IQ-8) (8.00 mg, 15 μmol, 34%, 99% Purity) as a clear white powder. m/z 516.1 (M+H)⁺ (ES+). ¹H NMR (400 MHz, MeOD) δ=8.41 (s, 1H), 8.24 (dt, J=7.9, 1.4 Hz, 1H), 8.09 (t, J=1.7 Hz, 1H), 7.82 (dd, J=8.6, 5.5 Hz, 1H), 7.63-7.54 (m, 2H), 7.52-7.40 (m, 2H), 7.32 (d, J=6.3 Hz, 1H), 4.16 (td, J=4.4, 2.4 Hz, 1H), 4.01-3.91 (m, 2H), 3.21 (s, 3H), 3.02-2.92 (m, 1H), 2.59 (d, J=1.0 Hz, 3H), 1.99-1.68 (m, 4H), 1.66-1.46 (m, 2H). Exchangeable NH & OH not observed.

### Example 362: N-((6-Fluoro-2-(4′-fluoro-2′-(4-methyl-4H-1,2,4-triazol-3-yl)-[1,1′-biphenyl]-3-yl)benzo[d]oxazol-5-yl)methyl)-2-methoxyethan-1-amine (IR-1)

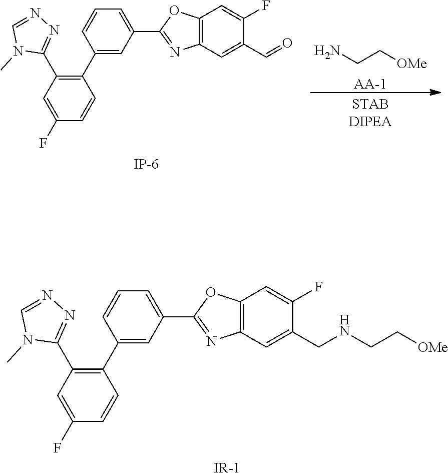

A solution of intermediate (IP-6) (40.0 mg, 1 Eq, 96.1 μmol), 2-methoxyethan-1-amine (AA-1) (14.4 mg, 16.7 μL, 2 Eq, 192 μmol) and DIPEA (62.1 mg, 83.7 μL, 5 Eq, 480 μmol) in CHCl₃ (1 mL) was stirred at 40° C. for 48 h. NaBH(OAc)₃ (102 mg, 5 Eq, 480 μmol) was added and the reaction mixture was stirred at 40° C. for another 2 h. The reaction mixture was diluted with sat. NaHCO₃ (10 mL) and transferred into a separating funnel. The layer was extracted with DCM (3×10 mL). The combined extracted layers were collected, dried (Na₂SO₄), filtered and concentrated in vacuo. The crude product was purified by chromatography on silica gel (4 g cartridge, 0-10% MeOH/DCM) to afford the title compound (IR-1) (25.0 mg, 52 μmol, 54%, 99% Purity) as an off-white powder. m/z 476.1 (M+H)⁺ (ES+). ¹H NMR (400 MHz, MeOD) δ=8.40 (s, 1H), 8.21 (dt, J=7.8, 1.5 Hz, 1H), 8.06 (t, J=1.8 Hz, 1H), 7.86 (d, J=6.7 Hz, 1H), 7.80 (dd, J=8.7, 5.5 Hz, 1H), 7.63-7.53 (m, 3H), 7.53-7.42 (m, 2H), 4.14 (d, J=1.5 Hz, 2H), 3.61 (t, J=5.2 Hz, 2H), 3.41 (s, 3H), 3.21 (s, 3H), 3.00 (t, J=5.2 Hz, 2H). Exchangeable NH not observed.

### Example 363: N-((7-Fluoro-2-(4′-fluoro-2′-(4-methyl-4H-1,2,4-triazol-3-yl)-[1,1′-biphenyl]-3-yl)benzo[d]oxazol-5-yl)methyl)-2-methoxyethan-1-amine (IS-1)

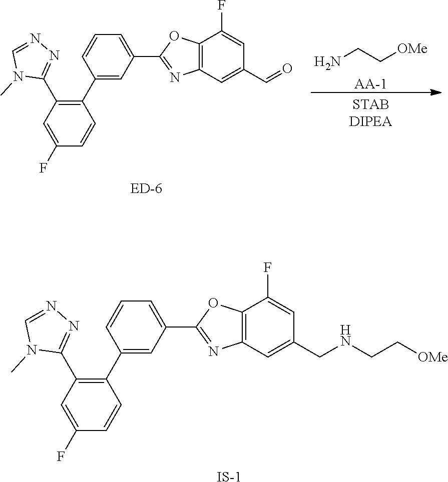

A solution of intermediate (ED-6) (39.5 mg, 70% Wt, 1 Eq, 66.4 μmol), 1-amino-2-methoxyethane (AA-1) (17.4 mg, 20.0 μL, 3.48 Eq, 231 μmol) and DIPEA (42.9 mg, 57.8 μL, 5 Eq, 332 μmol) in CHCl₃ (3.0 mL) was stirred at rt for 1 h. NaBH(OAc)₃ (60.5 mg, 97% Wt, 4.17 Eq, 277 μmol) was added and the reaction mixture was stirred at rt for 3 d. Additional 1-amino-2-methoxyethane (AA-1) (17.4 mg, 20.0 μL, 3.48 Eq, 231 μmol) and NaBH(OAc)₃ (56.8 mg, 97% Wt, 3.91 Eq, 260 μmol) were added and the resulting mixture was stirred at rt for 1 h. NaBH(OAc)₃ (50.8 mg, 97% Wt, 3.50 Eq, 232 μmol) was added and the mixture was stirred at rt overnight. The reaction mixture was diluted with EtOAc (2 mL), washed with sat. NaHCO₃ (2 mL) and brine (2 mL), dried (Na₂SO₄), filtered and concentrated in vacuo. The crude product was purified by chromatography on silica gel (4 g cartridge, 0-10% MeOH/DCM) to afford the title compound (IS-1) (21.3 mg, 44 μmol, 67%, 99% Purity) as an off-white solid. m/z 476.2 (M+H)⁺ (ES+). ¹H NMR (400 MHz, DMSO) δ 8.42 (s, 1H), 8.14 (d, J=8.0 Hz, 1H), 8.00-7.96 (m, 1H), 7.78 (dd, J=8.6, 5.6 Hz, 1H), 7.63-7.57 (m, 2H), 7.57-7.51 (m, 2H), 7.36 (d, J=11.3 Hz, 1H), 7.30 (d, J=8.0 Hz, 1H), 3.84 (s, 2H), 3.41 (t, J=5.7 Hz, 2H), 3.24 (s, 3H), 3.14 (s, 3H), 2.64 (t, J=5.8 Hz, 2H). 1× Exchangeable NH proton not observed.

### Example 364: (1S,2R)-2-(((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)(methyl-d3)amino)cyclopentan-1-ol (IT-1)

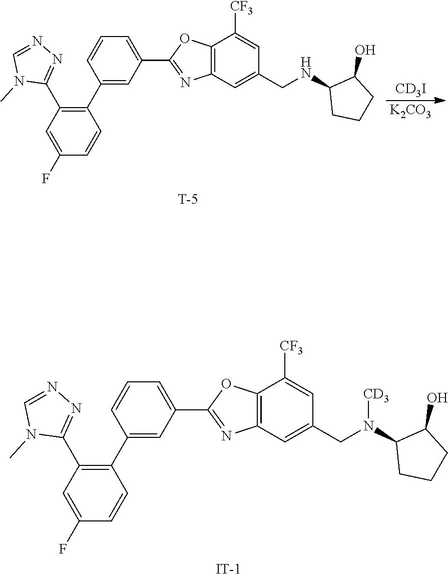

To a stirred solution of compound (T-5) (20.0 mg, 1 Eq, 36.3 μmol) in dry MeCN (1.00 mL) under a nitrogen atmosphere at 70° C. was added K₂CO₃ (10.0 mg, 2 Eq, 72.5 μmol) and CD₃I (7.88 mg, 3.54 μL, 1.5 Eq, 54.4 μmol). The reaction mixture was stirred for 5 h. The reaction mixture was diluted with distilled water (2 mL) and transferred into a separating funnel. The layer was extracted with EtOAc (3×5 mL). The combined extracted layers were collected, dried (Na₂SO₄), filtered and concentrated in vacuo. The crude product was purified by chromatography on silica gel (4 g cartridge, 0-10% (0.7 M ammonia/MeOH)/DCM) to afford the title compound (IT-1) (6.00 mg, 10 μmol, 29%, 99% Purity) as a clear white solid. m/z 569.2 (M+H)⁺ (ES+). ¹H NMR (400 MHz, MeOD) δ=8.41 (s, 1H), 8.23 (dt, J=7.9, 1.4 Hz, 1H), 8.08 (d, J=1.9 Hz, 1H), 8.03 (s, 1H), 7.84-7.76 (m, 2H), 7.58 (dt, J=11.5, 8.1 Hz, 2H), 7.53-7.43 (m, 2H), 4.28 (t, J=3.9 Hz, 1H), 3.91 (d, J=13.4 Hz, 1H), 3.77 (d, J=13.3 Hz, 1H), 3.27 (s, 3H), 2.62 (td, J=8.3, 4.7 Hz, 1H), 2.03-1.91 (m, 2H), 1.84 (dtd, J=18.4, 9.7, 3.0 Hz, 3H), 1.75-1.64 (m, 1H). Exchangeable OH not observed.

### Example 365: (S)-2-(4′-Fluoro-2′-(4-methyl-4H-1,2,4-triazol-3-yl)-[1,1′-biphenyl]-3-yl)-5-((3-methoxypiperidin-1-yl)methyl)-7-(trifluoromethyl)benzo[d]oxazole (IU-2)

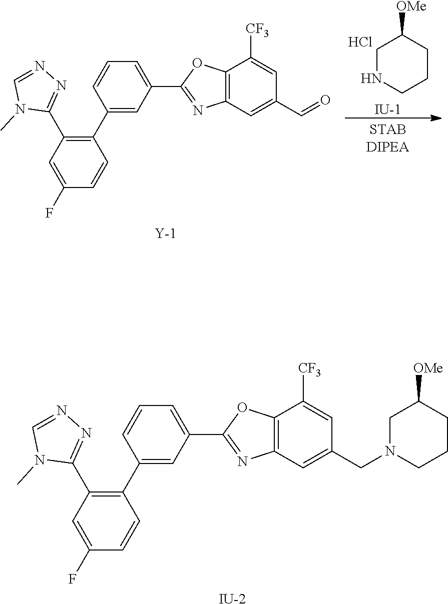

A solution of intermediate (Y-1) (23.0 mg, 1 Eq, 49.3 μmol), (3S)-3-methoxypiperidine, HCl (IU-1) (11.2 mg, 1.5 Eq, 74.0 μmol) and DIPEA (19.1 mg, 25.8 μL, 3 Eq, 148 μmol) in CHCl₃ (1 mL) was stirred for 12 h at 40° C. NaBH(OAc)₃ (41.8 mg, 4 Eq, 197 μmol) was added and stirring was continued at 40° C. for another 2 h. The reaction mixture was diluted with NaHCO₃ (25 mL) and transferred into a separating funnel. The layer was extracted with EtOAc (3×25 mL). The combined extracted layers were collected, dried (Na₂SO₄), filtered and concentrated in vacuo. The crude product was purified by chromatography on silica gel (4 g cartridge, 0-10% (0.7 M ammonia/MeOH)/DCM) to afford the title compound (IU-2) (7.00 mg, 12 μmol, 25%, 99% Purity) as a clear colourless solid. m/z 566.1 (M+H)⁺ (ES+). ¹H NMR (400 MHz, MeOD) δ=8.41 (s, 1H), 8.24 (dt, J=8.0, 1.3 Hz, 1H), 8.09 (t, J=1.8 Hz, 1H), 8.00 (d, J=1.4 Hz, 1H), 7.82 (dd, J=8.6, 5.5 Hz, 1H), 7.78-7.74 (m, 1H), 7.66-7.54 (m, 2H), 7.53-7.44 (m, 2H), 3.83-3.68 (m, 2H), 3.43-3.37 (m, 1H), 3.35 (s, 3H), 3.28 (s, 3H), 2.89 (d, J=11.2 Hz, 1H), 2.66 (s, 1H), 2.25 (t, J=10.2 Hz, 1H), 2.17 (t, J=9.6 Hz, 1H), 1.96 (s, 1H), 1.81 (s, 1H), 1.63-1.53 (m, 1H), 1.34 (d, J=14.3 Hz, 1H).

### Example 366: (R)-(1-((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)piperidin-2-yl)methanol (IV-2)

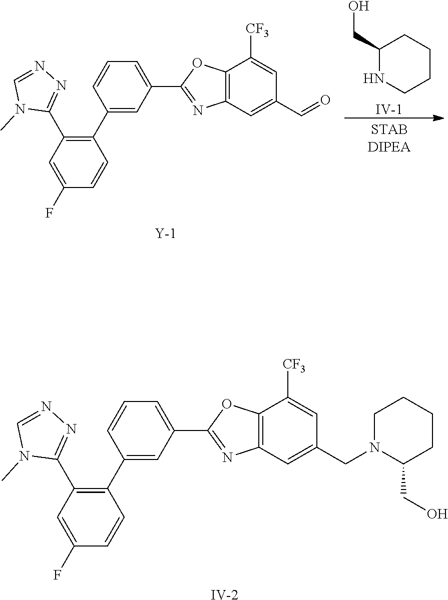

A solution of intermediate (Y-1) (20.0 mg, 1 Eq, 42.9 μmol), ((R)-piperidin-2-yl)methanol (IV-1) (9.88 mg, 2 Eq, 85.8 μmol) and DIPEA (16.6 mg, 22.4 μL, 3 Eq, 129 μmol) in CHCl₃ (2 mL) was stirred for 12 h at 40° C. NaBH(OAc)₃ (36.4 mg, 4 Eq, 172 μmol) was added and stirring was continued at 40° C. for another 2 h. The reaction mixture was diluted with NaHCO₃ (25 mL) and transferred into a separating funnel. The layer was extracted with EtOAc (3×25 mL). The combined extracted layers were collected, dried (Na₂SO₄), filtered and concentrated in vacuo. The crude product was purified by chromatography on silica gel (4 g cartridge, 0-10% MeOH/DCM) to afford the title compound (IV-2) (6.00 mg, 11 μmol, 25%, 99% Purity) as a clear white powder. m/z 566.1 (M+H)⁺ (ES+). ¹H NMR (400 MHz, MeOD) δ 8.40 (s, 1H), 8.29-8.16 (m, 1H), 8.07 (t, J=1.8 Hz, 1H), 8.02 (s, 1H), 7.83-7.75 (m, 2H), 7.62-7.54 (m, 2H), 7.51-7.43 (m, 2H), 4.37 (d, J=13.8 Hz, 1H), 3.82 (dd, J=11.4, 4.5 Hz, 1H), 3.74 (dd, J=11.4, 4.9 Hz, 1H), 3.55 (d, J=13.9 Hz, 1H), 3.26 (s, 3H), 2.80 (d, J=11.8 Hz, 1H), 2.46 (s, 1H), 2.16 (t, J=10.9 Hz, 1H), 1.78 (s, 2H), 1.63-1.48 (m, 3H), 1.40 (d, J=11.7 Hz, 1H). Exchangeable OH not observed.

### Example 367: 1-((((6,7-Difluoro-2-(4′-fluoro-2′-(4-methyl-4H-1,2,4-triazol-3-yl)-[1,1′-biphenyl]-3-yl)benzo[d]oxazol-5-yl)methyl)amino)methyl)cyclobutan-1-ol (IW-1)

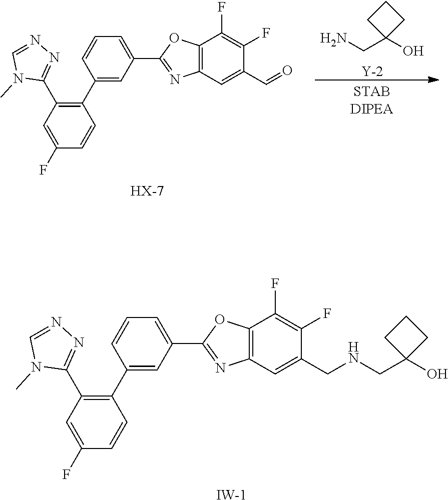

A solution of intermediate (HX-7) (50.6 mg, 50% Wt, 1 Eq, 58.2 μmol), 1-aminomethyl-cyclobutanol (Y-2) (17.1 mg, 2.90 Eq, 169 μmol) and DIPEA (52 mg, 70 μL, 6.9 Eq, 0.40 mmol) in CHCl₃ (2.0 mL) was stirred at rt for 1 h. NaBH(OAc)₃ (57.6 mg, 97% Wt, 4.53 Eq, 264 μmol) was added and the reaction mixture was stirred at 30° C. overnight. NaBH(OAc)₃ (55.1 mg, 97% Wt, 4.33 Eq, 252 μmol) was added and the reaction mixture was stirred at 30° C. for 3 h. The reaction mixture was diluted with EtOAc (5 mL), washed with sat. NaHCO₃ (5 mL) and brine (5 mL), dried (Na₂SO₄), filtered and concentrated in vacuo. The crude product was purified by chromatography on silica gel (4 g cartridge, 0-10% MeOH/DCM) to afford the title compound (IW-1) (8.4 mg, 16 μmol, 27%, 99% Purity) as an off-white solid. m/z 520.1 (M+H)⁺ (ES+). ¹H NMR (400 MHz, MeOD) δ 8.38 (s, 1H), 8.21-8.15 (m, 1H), 8.09-8.04 (m, 1H), 7.80-7.73 (m, 1H), 7.64 (d, J=5.3 Hz, 1H), 7.59-7.51 (m, 2H), 7.46 (dd, J=8.7, 2.7 Hz, 1H), 7.43-7.37 (m, 1H), 4.02 (s, 2H), 3.19 (s, 3H), 2.75 (s, 2H), 2.16-1.98 (m, 4H), 1.81-1.69 (m, 1H), 1.61-1.48 (m, 1H). 2× Exchangeable protons not observed.

### Example 368: 1-Cyclobutyl-N-((6,7-difluoro-2-(4′-fluoro-2′-(4-methyl-4H-1,2,4-triazol-3-yl)-[1,1′-biphenyl]-3-yl)benzo[d]oxazol-5-yl)methyl)methanamine (IX-1)

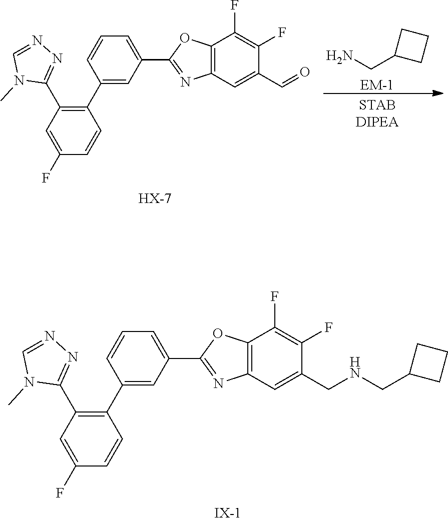

A solution of intermediate (HX-7) (76.6 mg, 50% Wt, 1 Eq, 88.2 μmol), (cyclobutylmethyl)amine (EM-1) (22 mg, 25 μL, 3.0 Eq, 0.26 mmol) and DIPEA (52 mg, 70 μL, 4.6 Eq, 0.40 mmol) in CHCl₃ (2.0 mL) was stirred at rt for 1 h. NaBH(OAc)₃ (57.0 mg, 97% Wt, 2.96 Eq, 261 μmol) was added and the reaction mixture was stirred at rt overnight. (Cyclobutylmethyl)amine (EM-1) (22 mg, 25 μL, 3.0 Eq, 0.26 mmol) and NaBH(OAc)₃ (51.8 mg, 97% Wt, 4.41 Eq, 389 μmol) was added and the reaction mixture was stirred at rt for 3 h. Further NaBH(OAc)₃ (59.0 mg, 97% Wt, 5.02 Eq, 443 μmol) was added and the resulting mixture was stirred at 30° C. overnight. The reaction mixture was diluted with EtOAc (5 mL), washed with sat. NaHCO₃ (5 mL) and brine (5 mL), dried (Na₂SO₄), filtered and concentrated in vacuo. The crude product was purified by chromatography on silica gel (4 g cartridge, 0-10% MeOH/DCM) to afford the title compound (IX-1) (22.9 mg, 45 μmol, 51%, 99% Purity) as a yellow solid. m/z 504.1 (M+H)⁺ (ES+). ¹H NMR (400 MHz, MeOD) δ 8.38 (s, 1H), 8.22-8.18 (m, 1H), 8.09-8.07 (m, 1H), 7.78 (dd, J=8.6, 5.4 Hz, 1H), 7.64-7.59 (m, 1H), 7.58-7.51 (m, 2H), 7.46 (dd, J=8.7, 2.8 Hz, 1H), 7.43-7.39 (m, 1H), 3.94 (s, 2H), 3.19 (s, 3H), 2.67 (d, J=7.3 Hz, 2H), 2.59-2.49 (m, 1H), 2.15-2.05 (m, 2H), 1.98-1.81 (m, 2H), 1.76-1.67 (m, 2H). 1× Exchangeable NH proton not observed.

### Example 369: N-((6,7-Difluoro-2-(4′-fluoro-2′-(4-methyl-4H-1,2,4-triazol-3-yl)-[1,1′-biphenyl]-3-yl)benzo[d]oxazol-5-yl)methyl)-2-methoxyethan-1-amine (IY-1)

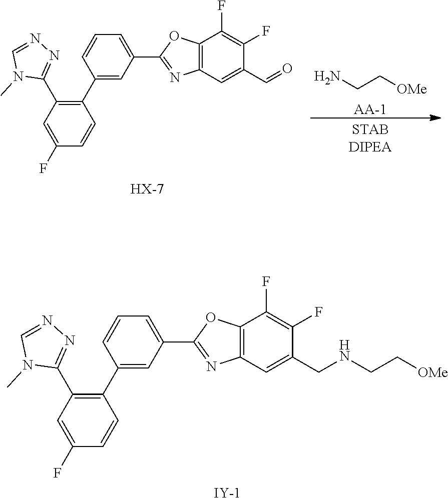

A solution of intermediate (HX-7) (58.6 mg, 50% Wt, 1 Eq, 67.5 μmol), 1-amino-2-methoxyethane (AA-1) (17 mg, 20 μL, 3.4 Eq, 0.23 mmol) and DIPEA (52 mg, 70 μL, 6.0 Eq, 0.40 mmol) in CHCl₃ (2.0 mL) was stirred at rt for 1 h. NaBH(OAc)₃ (57.4 mg, 97% Wt, 3.89 Eq, 263 μmol) was added and the reaction mixture was stirred at rt overnight. Additional 1-amino-2-methoxyethane (AA-1) (17 mg, 20 μL, 3.4 Eq, 0.23 mmol) and NaBH(OAc)₃ (57.4 mg, 97% Wt, 3.89 Eq, 263 μmol) was added and the reaction mixture was stirred at rt for 3 h. Further NaBH(OAc)₃ (56.3 mg, 97% Wt, 3.82 Eq, 258 μmol) was added and the resulting mixture was stirred at 30° C. overnight. The reaction mixture was diluted with EtOAc (5 mL), washed with sat. NaHCO₃ (5 mL) and brine (5 mL), dried (Na₂SO₄), filtered and concentrated in vacuo. The crude product was purified by chromatography on silica gel (4 g cartridge, 0-10% MeOH/DCM) to afford product with ˜90% purity. The crude product was purified by preparative HPLC (Waters, Basic (0.3% ammonia), Basic, Waters X-Bridge BEH C18 ODB, 5 μm, 30×100 mm column 30-100% MeCN in water) to afford the title compound (IY-1) (12.8 mg, 26 μmol, 38%, 99% Purity) as a white solid. m/z 494.1 (M+H)⁺ (ES+). ¹H NMR (400 MHz, MeOD) δ 8.38 (s, 1H), 8.19 (dt, J=7.8, 1.4 Hz, 1H), 8.08 (t, J=1.8 Hz, 1H), 7.77 (dd, J=8.7, 5.4 Hz, 1H), 7.61 (dd, J=5.5, 1.7 Hz, 1H), 7.58-7.51 (m, 2H), 7.46 (dd, J=8.8, 2.7 Hz, 1H), 7.41 (ddd, J=7.9, 1.4, 1.4 Hz, 1H), 3.98 (d, J=1.7 Hz, 2H), 3.53 (t, J=5.3 Hz, 2H), 3.36 (s, 3H), 3.19 (s, 3H), 2.80 (t, J=5.3 Hz, 2H). 1× Exchangeable NH proton not observed.

### Example 370: 1-((((7-Fluoro-2-(4′-fluoro-2′-(4-methyl-4H-1,2,4-triazol-3-yl)-[1,1′-biphenyl]-3-yl)benzo[d]oxazol-5-yl)methyl)amino)methyl)cyclobutan-1-ol (IZ-1)

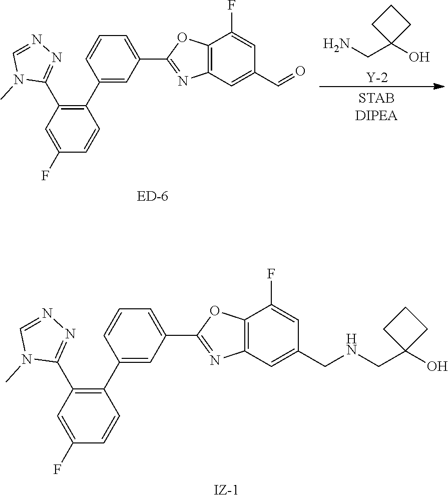

A solution of intermediate (ED-6) (30.3 mg, 70% Wt, 1 Eq, 50.9 μmol), 1-aminomethyl-cyclobutanol (Y-2) (20.2 mg, 3.92 Eq, 200 μmol) and DIPEA (45 mg, 60 μL, 6.8 Eq, 0.34 mmol) in CHCl₃ (2.0 mL) was stirred at 30° C. for 1 h. NaBH(OAc)₃ (48.8 mg, 97% Wt, 4.38 Eq, 223 μmol) was added and the reaction mixture was stirred at 30° C. overnight. NaBH(OAc)₃ (49.0 mg, 97% Wt, 4.40 Eq, 224 μmol) was added and the reaction mixture was stirred at 30° C. for 2 h. The reaction mixture was diluted with EtOAc (5 mL), washed with sat. NaHCO₃ (5 mL) and brine (5 mL), dried (Na₂SO₄), filtered and concentrated in vacuo. The crude product was purified by chromatography on silica gel (4 g cartridge, 0-20% MeOH/DCM) to afford product at 94% purity. The crude product was purified by preparative HPLC (Waters, Basic (0.3% ammonia), Basic, Waters X-Bridge BEH C18 ODB, 5 μm, 30×100 mm column 40-100% MeCN in water) to afford the title compound (8.3 mg, 16 μmol, 32%, 99% Purity) as a white solid. m/z 502.2 (M+H)⁺ (ES+). ¹H NMR (400 MHz, MeOD) δ 8.38 (s, 1H), 8.20 (dt, J=7.9, 1.4 Hz, 1H), 8.10 (t, J=1.8 Hz, 1H), 7.78 (dd, J=8.6, 5.5 Hz, 1H), 7.59-7.50 (m, 3H), 7.46 (dd, J=8.8, 2.8 Hz, 1H), 7.40 (dt, J=7.8, 1.4 Hz, 1H), 7.30 (dd, J=11.1, 1.4 Hz, 1H), 3.94 (s, 2H), 3.20 (s, 3H), 2.71 (s, 2H), 2.14-1.97 (m, 4H), 1.79-1.69 (m, 1H), 1.57-1.46 (m, 1H). 2× Exchangeable protons not observed.

### Example 371: 2-(2-(4′-Fluoro-2′-(4-methyl-4H-1,2,4-triazol-3-yl)-[1,1′-biphenyl]-3-yl)-7-(trifluoromethyl)benzo[d]oxazol-5-yl)propan-2-ol (JA-1)

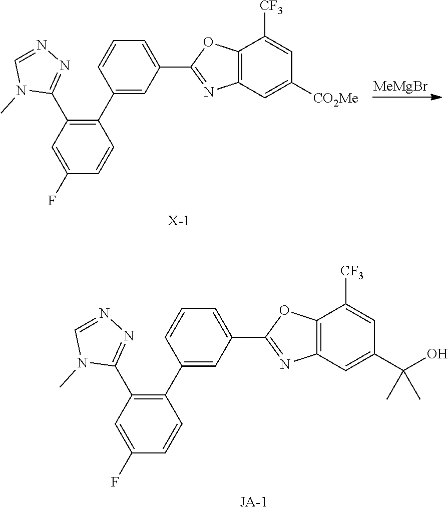 [SMILES: "MeMgBr" ; COC(=O)c1cc(C(F)(F)F)c2oc(-c3cccc(-c4ccc(F)cc4-c4nncn4C)c3)nc2c1 ; "X-1" ; Cn1cnnc1-c1cc(F)ccc1-c1cccc(-c2nc3cc(C(C)(C)O)cc(C(F)(F)F)c3o2)c1 ; "JA-1"]

To a stirred solution of intermediate (X-1) (50 mg, 1 Eq, 0.10 mmol) in dry THF (1.5 mL) under a nitrogen atmosphere at 0° C. was slowly added MeMgBr (36 mg, 0.10 mL, 3 molar, 3 Eq, 0.30 mmol). The reaction mixture was stirred for 1 h at the same temp. The reaction mixture was diluted with 1M HCl (5 mL) and transferred into a separating funnel. The layer was extracted with DCM (3×5 mL). The combined extracted layers were collected, dried (Na₂SO₄), filtered and concentrated in vacuo. The crude product was purified by chromatography on silica gel (24 g cartridge, 0-10% (0.7 M ammonia/MeOH)/DCM) to afford the title compound (JA-1) (30 mg, 60 μmol, 59%, 99.0% Purity) as a light brown solid. m/z 497.2 (M+H)⁺ (ES+). ¹H NMR (400 MHz, MeOD) δ 8.41 (s, 1H), 8.24 (dt, J=7.8, 1.4 Hz, 1H), 8.11 (dt, J=17.2, 1.5 Hz, 2H), 7.90 (s, 1H), 7.81 (dd, J=8.7, 5.4 Hz, 1H), 7.64-7.54 (m, 2H), 7.52-7.45 (m, 2H), 3.28 (s, 3H), 1.65 (s, 6H). Exchangeable proton not observed.

### Example 372: (S)-(1-((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)piperidin-2-yl)methanol (JB-2)

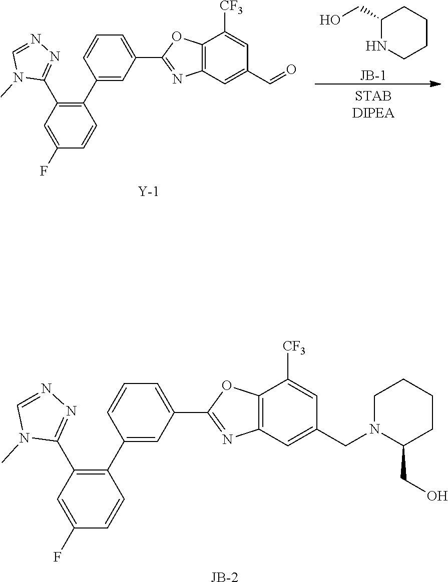

A solution of intermediate (Y-1) (30.0 mg, 94% Wt, 1 Eq, 60.5 μmol), (2S)-piperidin-2-ylmethanol (JB-1) (13.9 mg, 2 Eq, 121 μmol) and DIPEA (23.4 mg, 31.6 μL, 3 Eq, 181 μmol) in CHCl₃ (2 mL) was stirred for 48 h at 40° C. NaBH(OAc)₃ (51.3 mg, 4 Eq, 242 μmol) was added and stirring was continued at 40° C. for another 2 h. The reaction mixture was diluted with NaHCO₃ (25 mL) and transferred into a separating funnel. The layer was extracted with EtOAc (3×25 mL). The combined extracted layers were collected, dried (Na₂SO₄), filtered and concentrated in vacuo. The crude product was purified by chromatography on silica gel (4 g cartridge, 0-10% MeOH/DCM) to afford the title compound (JB-2) (28.0 mg, 49 μmol, 81%, 99% Purity) as a clear white powder. m/z 566.2 (M+H)⁺ (ES+). ¹H NMR (400 MHz, MeOD) δ=8.41 (s, 1H), 8.24 (d, J=8.0 Hz, 1H), 8.09 (t, J=1.8 Hz, 1H), 8.03 (s, 1H), 7.81 (dd, J=8.7, 5.4 Hz, 1H), 7.78 (s, 1H), 7.64-7.54 (m, 2H), 7.48 (ddd, J=11.1, 8.5, 2.2 Hz, 2H), 4.38 (d, J=13.9 Hz, 1H), 3.87-3.71 (m, 2H), 3.56 (d, J=13.8 Hz, 1H), 3.28 (s, 3H), 2.82 (d, J=11.9 Hz, 1H), 2.47 (s, 1H), 2.17 (t, J=10.4 Hz, 1H), 1.80 (s, 2H), 1.65-1.50 (m, 3H), 1.42 (s, 1H). Exchangeable OH not observed.

### Example 373: N-((4-Fluoro-2-(4′-fluoro-2′-(4-methyl-4H-1,2,4-triazol-3-yl)-[1,1′-biphenyl]-3-yl)-7-methylbenzo[d]oxazol-5-yl)methyl)-2-methoxyethan-1-amine (JC-1)

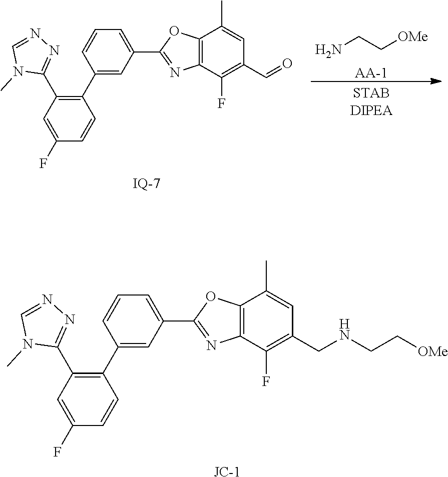

A solution of intermediate (IQ-7) (35.0 mg, 79% Wt, 1 Eq, 64.2 μmol), 2-methoxyethan-1-amine (AA-1) (9.65 mg, 11.2 μL, 2 Eq, 128 μmol) and DIPEA (41.5 mg, 56.0 μL, 5 Eq, 321 μmol) in CHCl₃ (1 mL) was stirred at 40° C. for 48 h. NaBH(OAc)₃ (68.1 mg, 5 Eq, 321 μmol) was added and the reaction mixture was stirred at 40° C. for another 2 h. The reaction mixture was diluted with Sat. NaHCO₃ (10 mL) and transferred into a separating funnel. The layer was extracted with DCM (3×10 mL). The combined extracted layers were collected, dried (Na₂SO₄), filtered and concentrated in vacuo. The crude product was purified by chromatography on silica gel (4 g cartridge, 0-10% MeOH/DCM) to afford the title compound (JC-1) (7.00 mg, 14 μmol, 22%, 99% Purity) as an off-white powder. m/z 490.1 (M+H)⁺ (ES+). ¹H NMR (400 MHz, MeOD) δ=8.55 (s, 1H), 8.41 (s, 1H), 8.26 (dt, J=7.8, 1.4 Hz, 1H), 8.08 (t, J=1.8 Hz, 1H), 7.82 (dd, J=8.6, 5.4 Hz, 1H), 7.65-7.54 (m, 2H), 7.50 (ddd, J=8.8, 4.3, 2.0 Hz, 2H), 7.35 (d, J=6.3 Hz, 1H), 4.24 (s, 2H), 3.71-3.56 (m, 2H), 3.43 (s, 3H), 3.22 (s, 3H), 3.12 (t, J=5.2 Hz, 2H), 2.62 (s, 3H).

### Example 374: 1-(((6,7-Difluoro-2-(4′-fluoro-2′-(4-methyl-4H-1,2,4-triazol-3-yl)-[1,1′-biphenyl]-3-yl)benzo[d]oxazol-5-yl)methyl)amino)-2-methylpropan-2-ol (JD-1)

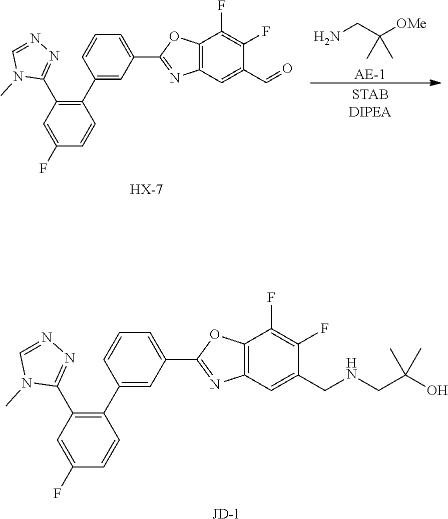

A solution of intermediate (HX-7) (30.7 mg, 49% Wt, 1 Eq, 34.6 μmol), 1-Amino-2,2-difluoro-1-methylcyclopropane (AE-1) (14 mg, 15 μL, 4.6 Eq, 0.16 mmol) and DIPEA (52 mg, 70 μL, 12 Eq, 0.40 mmol) in CHCl₃ (2.0 mL) was stirred at rt for 1 h. NaBH(OAc)₃ (77.3 mg, 97% Wt, 10.2 Eq, 354 μmol) was added and the reaction mixture was stirred at rt overnight. The reaction mixture was diluted with EtOAc (5 mL), washed with sat. NaHCO₃ (5 mL) and brine (5 mL), dried (Na₂SO₄), filtered and concentrated in vacuo. The crude product was purified by chromatography on silica gel (4 g cartridge, 0-10% MeOH/DCM) to afford product with ˜90% purity. The crude product was purified by preparative HPLC (Waters, Basic (0.3% ammonia), Basic, Waters X-Bridge BEH C18 ODB, 5 μm, 30×100 mm column 40-100% MeCN in water) to afford the title compound (JD-1) (5.7 mg, 11 μmol, 32%, 99% Purity) as a white solid. m/z 508.1 (M+H)⁺ (ES+). ¹H NMR (400 MHz, MeOD) δ 8.38 (s, 1H), 8.19 (ddd, J=7.9, 1.5, 1.5 Hz, 1H), 8.09-8.05 (m, 1H), 7.77 (dd, J=8.6, 5.4 Hz, 1H), 7.64 (dd, J=5.5, 1.6 Hz, 1H), 7.59-7.51 (m, 2H), 7.46 (dd, J=8.8, 2.8 Hz, 1H), 7.41 (ddd, J=7.7, 1.4, 1.4 Hz, 1H), 3.99 (s, 2H), 3.20 (s, 3H), 2.57 (s, 2H), 1.22 (s, 6H). 2× Exchangeable protons not observed.

### Example 375: 1-(6,7-Difluoro-2-(4′-fluoro-2′-(4-methyl-4H-1,2,4-triazol-3-yl)-[1,1′-biphenyl]-3-yl)benzo[d]oxazol-5-yl)-N-(oxetan-3-ylmethyl)methanamine (JE-1)

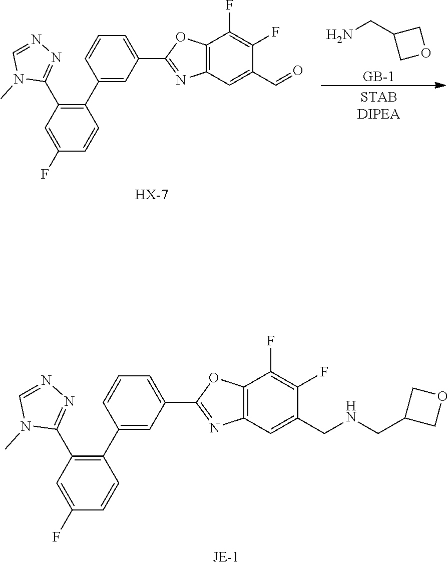

A solution of intermediate (HX-7) (31.5 mg, 49% Wt, 1 Eq, 35.5 μmol), 3-Oxetanemethylamine (GB-1) (15 mg, 15 μL, 98% Wt, 4.8 Eq, 0.17 mmol) and DIPEA (52 mg, 70 μL, 11 Eq, 0.40 mmol) in CHCl₃ (2.0 mL) was stirred at rt for 1 h. NaBH(OAc)₃ (81.0 mg, 97% Wt, 10.4 Eq, 371 μmol) was added and the reaction mixture was stirred at rt overnight. The reaction mixture was diluted with EtOAc (5 mL), washed with sat. NaHCO₃ (5 mL) and brine (5 mL), dried (Na₂SO₄), filtered and concentrated in vacuo. The crude product was purified by chromatography on silica gel (4 g cartridge, 0-10% MeOH/DCM) to afford product with ˜90% purity. The crude product was purified by preparative HPLC (Waters, Basic (0.3% ammonia), Basic, Waters X-Bridge BEH C18 ODB, 5 μm, 30×100 mm column 27.5-100% MeCN in water) to afford the title compound (JE-1) (3.5 mg, 6.9 μmol, 19%, 99% Purity) as a white solid. m/z 506.1 (M+H)⁺ (ES+). ¹H NMR (400 MHz, MeOD) δ 8.38 (s, 1H), 8.22-8.16 (m, 1H), 8.09-8.06 (m, 1H), 7.77 (dd, J=8.6, 5.5 Hz, 1H), 7.62 (d, J=5.7 Hz, 1H), 7.59-7.50 (m, 2H), 7.46 (dd, J=8.8, 2.8 Hz, 1H), 7.44-7.38 (m, 1H), 4.81 (dd, J=7.8, 6.1 Hz, 2H), 4.42 (t, J=6.1 Hz, 2H), 3.95 (d, J=1.6 Hz, 2H), 3.19 (s, 3H), 3.18-3.12 (m, 1H), 2.95 (d, J=7.5 Hz, 2H). 1× Exchangeable NH proton not observed.

### Example 376: N-((6,7-Difluoro-2-(4′-fluoro-2′-(4-methyl-4H-1,2,4-triazol-3-yl)-[1,1′-biphenyl]-3-yl)benzo[d]oxazol-5-yl)methyl)cyclobutanamine (JF-1)

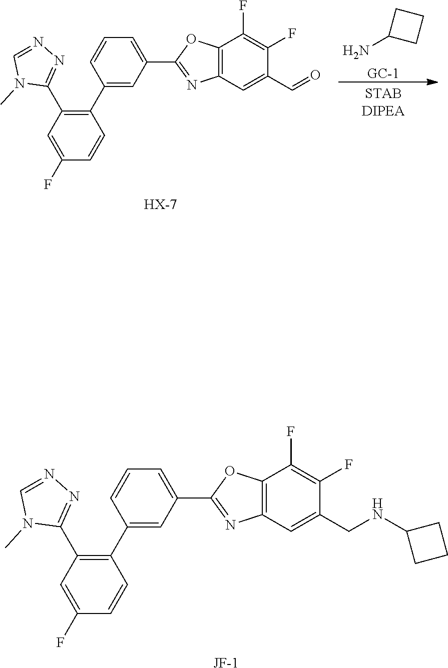

A solution of intermediate (HX-7) (33.8 mg, 49% Wt, 1 Eq, 38.1 μmol), cyclobutylamine (GC-1) (12 mg, 15 μL, 98% Wt, 4.5 Eq, 0.17 mmol) and DIPEA (52 mg, 70 μL, 11 Eq, 0.40 mmol) in CHCl₃ (2.0 mL) was stirred at rt for 1 h. NaBH(OAc)₃ (89.6 mg, 97% Wt, 10.8 Eq, 410 μmol) was added and the reaction mixture was stirred at rt overnight. The reaction mixture was diluted with EtOAc (5 mL), washed with sat. NaHCO₃ (5 mL) and brine (5 mL), dried (Na₂SO₄), filtered and concentrated in vacuo. The crude product was purified by chromatography on silica gel (4 g cartridge, 0-10% MeOH/DCM) to afford product with ˜90% purity. The crude product was purified by preparative HPLC (Waters, Basic (0.3% ammonia), Basic, Waters X-Bridge BEH C18 ODB, 5 μm, 30×100 mm column 40-100% MeCN in water) to afford the title compound (JF-1) (4.9 mg, 9.9 μmol, 26%, 99% Purity) as a white solid. m/z 490.0 (M+H)⁺ (ES+). ¹H NMR (400 MHz, MeOD) δ 8.38 (s, 1H), 8.19 (dt, J=7.8, 1.4 Hz, 1H), 8.08 (t, J=1.8 Hz, 1H), 7.77 (dd, J=8.7, 5.4 Hz, 1H), 7.61-7.50 (m, 3H), 7.46 (dd, J=8.8, 2.7 Hz, 1H), 7.41 (dt, J=7.9, 1.5 Hz, 1H), 3.86 (d, J=1.8 Hz, 2H), 3.37-3.32 (m, 1H), 3.19 (s, 3H), 2.24-2.13 (m, 2H), 1.87-1.76 (m, 2H), 1.76-1.63 (m, 2H). 1× Exchangeable NH proton not observed.

### Example 377: 1-((((7-Cyclopropyl-2-(4′-fluoro-2′-(4-methyl-4H-1,2,4-triazol-3-yl)-[1,1′-biphenyl]-3-yl)benzo[d]oxazol-5-yl)methyl)amino)methyl)cyclobutan-1-ol (JG-1)

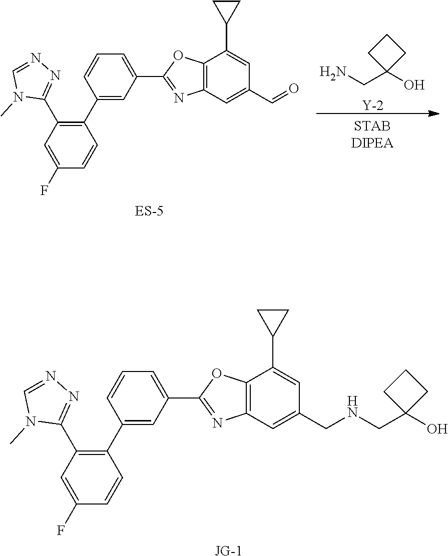

A solution of intermediate (ES-5) (18.0 mg, 85% Wt, 1 Eq, 34.9 μmol), 1-(aminomethyl)cyclobutan-1-ol (Y-2) (7.06 mg, 2 Eq, 69.8 μmol) and DIPEA (13.5 mg, 18.2 μL, 3 Eq, 105 μmol) in CHCl₃ (2 mL) was stirred for 12 h at 40° C. NaBH(OAc)₃ (29.6 mg, 4 Eq, 140 μmol) was added and stirring was continued at 40° C. for another 2 h. The reaction mixture was diluted with NaHCO₃ (25 mL) and transferred into a separating funnel. The layer was extracted with EtOAc (3×25 mL). The combined extracted layers were collected, dried (Na₂SO₄), filtered and concentrated in vacuo. The crude product was purified by chromatography on silica gel (4 g cartridge, 0-10% MeOH/DCM) to afford the title compound (JG-1) (6.00 mg, 11 μmol, 33%, 99% Purity) as a clear white powder. m/z 524.2 (M+H)⁺ (ES+). ¹H NMR (400 MHz, MeOD) δ 8.41 (s, 1H), 8.21 (dt, J=7.9, 1.5 Hz, 1H), 8.05 (t, J=1.8 Hz, 1H), 7.81 (dd, J=8.6, 5.4 Hz, 1H), 7.62-7.54 (m, 2H), 7.54-7.42 (m, 3H), 7.11 (d, J=1.6 Hz, 1H), 3.93 (s, 2H), 3.21 (s, 3H), 2.74 (s, 2H), 2.36 (tt, J=8.4, 5.2 Hz, 1H), 2.17-2.01 (m, 4H), 1.83-1.71 (m, 1H), 1.54 (dp, J=11.3, 8.9 Hz, 1H), 1.23-1.13 (m, 2H), 1.10-1.02 (m, 2H). 2 Exchangeable protons not observed.

### Example 378: N-((7-Cyclopropyl-2-(4′-fluoro-2′-(4-methyl-4H-1,2,4-triazol-3-yl)-[1,1′-biphenyl]-3-yl)benzo[d]oxazol-5-yl)methyl)-2-methoxyethan-1-amine (JH-1)

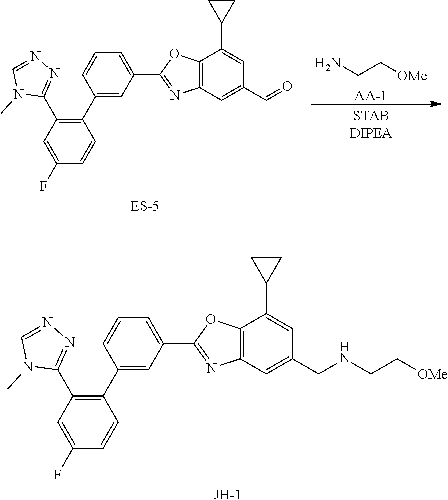

A solution of intermediate (ES-5) (22.0 mg, 85% Wt, 1 Eq, 42.6 μmol), 2-methoxyethan-1-amine (AA-1) (6.41 mg, 2 Eq, 85.3 μmol) and DIPEA (16.5 mg, 22.3 μL, 3 Eq, 128 μmol) in CHCl₃ (2 mL) was stirred for 48 h at 40° C. NaBH(OAc)₃ (36.2 mg, 4 Eq, 171 μmol) was added and stirring was continued at 40° C. for another 2 h. The reaction mixture was diluted with NaHCO₃ (25 mL) and transferred into a separating funnel. The layer was extracted with EtOAc (3×25 mL). The combined extracted layers were collected, dried (Na₂SO₄), filtered and concentrated in vacuo. The crude product was purified by chromatography on silica gel (4 g cartridge, 0-10% MeOH/DCM) to afford the title compound (JH-1) (6.00 mg, 12 μmol, 28%, 99% Purity) as a clear white powder. m/z 498.2 (M+H)⁺ (ES+). ¹H NMR (400 MHz, MeOD) δ=8.37 (s, 1H), 8.18 (dt, J=7.8, 1.4 Hz, 1H), 8.01 (t, J=1.8 Hz, 1H), 7.77 (dd, J=8.6, 5.5 Hz, 1H), 7.60-7.50 (m, 2H), 7.48-7.38 (m, 3H), 7.05 (d, J=1.6 Hz, 1H), 3.84 (s, 2H), 3.51 (t, J=5.4 Hz, 2H), 3.34 (s, 3H), 3.17 (s, 3H), 2.76 (t, J=5.4 Hz, 2H), 2.32 (tt, J=8.4, 5.1 Hz, 1H), 1.20-1.09 (m, 2H), 1.07-0.98 (m, 2H). Exchangeable proton not observed.

### Example 379: (1S,2R)-2-(((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)amino)-1-(trifluoromethyl)cyclopentan-1-ol (JI-3)

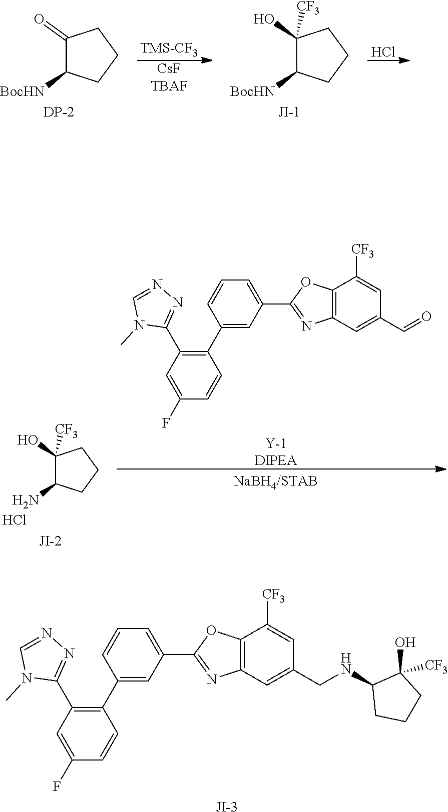

Step 1: tert-Butyl ((1R,2S)-2-hydroxy-2-(trifluoromethyl)cyclopentyl)carbamate (JI-1). To a stirred solution of intermediate (DP-2) (665.8 mg, 75% Wt, 1 Eq, 2.506 mmol) in dry DME (10 mL) under a nitrogen atmosphere at 0° C. was added trimethyl(trifluoromethyl)silane (712 mg, 750 μL, 2.00 Eq, 5.01 mmol). The reaction mixture was stirred at 0° C. for 30 min and then at rt for 3 h. The reaction mixture was cooled to 0° C. A solution of TBAF in THF (4.56 g, 5.00 mL, 1.00 molar, 2.00 Eq, 5.00 mmol) and trimethyl(trifluoromethyl)silane (712 mg, 750 μL, 2.00 Eq, 5.01 mmol) were added. The resulting mixture was stirred at 0° C. and gradually warmed to rt overnight. The reaction mixture was diluted with brine (5 mL), extracted with EtOAc (3×5 mL). The combined organic layers were collected, dried (Na₂SO₄), filtered and concentrated in vacuo. The crude product was purified by chromatography on silica gel (12 g cartridge, 0-10% MeOH/DCM) to afford the sub-title compound (JI-1) (72.9 mg, 0.24 mmol, 9.7%, 90% Purity) as a brown oil. ¹H NMR (400 MHz, DMSO) δ 6.24 (d, J=9.5 Hz, 1H), 5.87 (s, 1H), 4.02 (q, J=9.4 Hz, 1H), 2.01-1.83 (m, 2H), 1.82-1.69 (m, 1H), 1.66 (t, J=8.1 Hz, 1H), 1.63-1.45 (m, 2H), 1.37 (s, 9H).

Step 2: (1S,2R)-2-Amino-1-(trifluoromethyl)cyclopentan-1-ol, HCl (JI-2). To a stirred solution of the product from step 1 above (JI-1) (42.5 mg, 90% Wt, 1 Eq, 142 μmol) in dry DCM (2.0 mL) at rt was added HCl in 1,4-dioxane (596 mg, 400 μL, 4.00 molar, 11.3 Eq, 1.60 mmol). The reaction mixture was stirred at rt overnight. The volatiles were removed in vacuo to afford the sub-title compound (JI-2) (62.5 mg, 0.14 mmol, 100%, 47% Purity) as a dark brown solid. ¹H NMR (400 MHz, MeOD) δ 3.75-3.69 (m, 1H), 2.32-2.16 (m, 2H), 2.00-1.94 (m, 2H), 1.91-1.80 (m, 1H), 1.80-1.70 (m, 1H). Exchangeable protons not observed.

Step 3: (1S,2R)-2-(((2-(4′-Fluoro-2′-(4-methyl-4H-1,2,4-triazol-3-yl)-[1,1′-biphenyl]-3-yl)-7-(trifluoromethyl)benzo[d]oxazol-5-yl)methyl)amino)-1-(trifluoromethyl)cyclopentan-1-ol (JI-3). A solution of intermediate (Y-1) (50.4 mg, 94% Wt, 1 Eq, 102 μmol), the product from step 2 above (JI-2) (62.5 mg, 47% Wt, 1.41 Eq, 143 μmol) and DIPEA (263 mg, 354 μL, 20 Eq, 2.03 mmol) in CHCl₃ (2.0 mL) was stirred at rt for 3 h, heated to 45° C. for 3 h and then stirred at rt overnight. The reaction mixture was heated to 45° C. for 6 h and then at rt overnight. NaBH₄ (5.0 mg, 1.3 Eq, 0.13 mmol) was added and the resulting mixture was stirred at rt overnight. NaBH(OAc)₃ (80.5 mg, 97% Wt, 3.63 Eq, 368 μmol) was added and the resulting mixture was stirred at rt for 2 h. The reaction mixture was then heated to 45° C. for 3 h. The reaction mixture was cooled to rt, diluted with EtOAc (5 mL), washed with sat. NaHCO₃ (5 mL) and brine (5 mL), dried (Na₂SO₄), filtered and concentrated in vacuo. The crude product was purified by chromatography on silica gel (4 g cartridge, 0-10% MeOH/DCM) to afford impure product. The crude product was purified by preparative HPLC (Waters, acidic (0.1% formic acid), acidic, Waters X-Select CSH C18 ODB prep column, 130A, 5 μm, 30 mm×100 mm column, 17.5-100% MeCN in water) to afford the title compound (JI-3) (2.1 mg, 3.2 μmol, 3.2%, 95% Purity) as a white solid. m/z 620.3 (M+H)⁺ (ES+). ¹H NMR (400 MHz, MeOD) δ 8.38 (s, 1H), 8.21 (dt, J=7.8, 1.4 Hz, 1H), 8.06 (t, J=1.8 Hz, 1H), 8.00-7.95 (m, 1H), 7.79 (dd, J=8.7, 5.4 Hz, 1H), 7.75-7.71 (m, 1H), 7.61-7.51 (m, 2H), 7.49-7.40 (m, 2H), 4.01 (s, 2H), 3.25 (s, 3H), 3.17 (t, J=8.1 Hz, 1H), 2.14-1.99 (m, 2H), 1.84-1.70 (m, 2H), 1.65-1.43 (m, 2H). 2× Exchangeable protons not observed.

### Example 380: (1S,2R)-2-(((6,7-Difluoro-2-(4′-fluoro-2′-(4-methyl-4H-1,2,4-triazol-3-yl)-[1,1′-biphenyl]-3-yl)benzo[d]oxazol-5-yl)methyl)(methyl)amino)cyclopentan-1-ol (JJ-3)

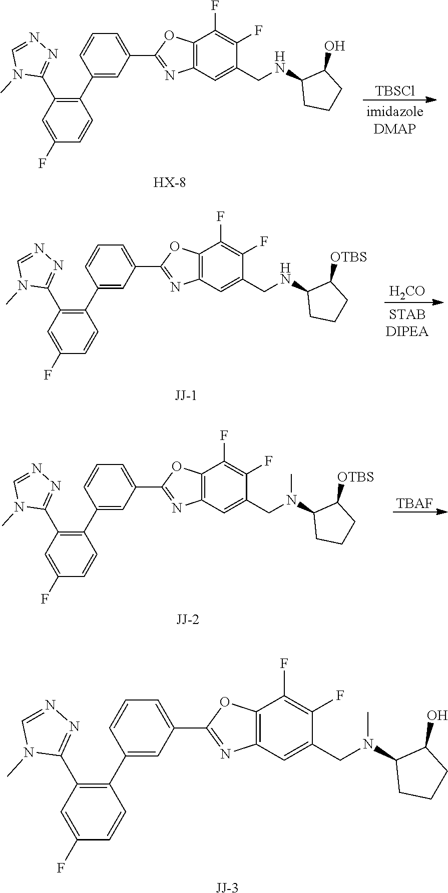

Step 1: (1R,2S)-2-((tert-Butyldimethylsilyl)oxy)-N-((6,7-difluoro-2-(4′-fluoro-2′-(4-methyl-4H-1,2,4-triazol-3-yl)-[1,1′-biphenyl]-3-yl)benzo[d]oxazol-5-yl)methyl)cyclopentan-1-amine (JJ-1). To a stirred solution of compound (HX-8) (49.7 mg, 99% Wt, 1 Eq, 94.7 μmol), imidazole (40.1 mg, 6.22 Eq, 589 μmol) and DMAP (5.6 mg, 0.48 Eq, 46 μmol) in dry DMF (1.0 mL) under a nitrogen atmosphere at rt was added tert-butyldimethylchlorosilane (37.0 mg, 42.1 μL, 2.59 Eq, 245 μmol). The reaction mixture was stirred at rt for 3 d. The reaction mixture was diluted with EtOAc (2 mL) and distilled water (2 mL). The aqueous layer was extracted with EtOAc (3×5 mL). The combined organic layers were collected, dried (Na₂SO₄), filtered and concentrated in vacuo. The crude product was purified by chromatography on silica gel (4 g cartridge, 0-10% MeOH/DCM) to afford the sub-title compound (JJ-1) (88.2 mg, 90 μmol, 96%, 65% Purity) as a white solid. m/z 634.2 (M+H)⁺ (ES+). ¹H NMR (400 MHz, MeOD) δ 8.38 (s, 1H), 8.20 (d, J=7.7 Hz, 1H), 8.10-8.06 (m, 1H), 7.78 (dd, J=8.7, 5.4 Hz, 1H), 7.59 (d, J=5.4 Hz, 1H), 7.58-7.51 (m, 2H), 7.46 (dd, J=8.8, 2.8 Hz, 1H), 7.40 (d, J=7.8 Hz, 1H), 4.27-4.22 (m, 1H), 3.96 (d, J=13.0 Hz, 1H), 3.94 (d, J=13.0 Hz, 1H), 3.19 (s, 3H), 3.00-2.93 (m, 1H), 1.94-1.67 (m, 4H), 1.66-1.51 (m, 2H), 0.91 (s, 9H), 0.10 (s, 3H), 0.08 (s, 3H). 1× Exchangeable NH proton not observed.

Step 2: (1R,2S)-2-((tert-butyldimethylsilyl)oxy)-N-((6,7-difluoro-2-(4′-fluoro-2′-(4-methyl-4H-1,2,4-triazol-3-yl)-[1,1′-biphenyl]-3-yl)benzo[d]oxazol-5-yl)methyl)-N-methylcyclopentan-1-amine (JJ-2). A solution of the product from step 1 above (JJ-1) (88.2 mg, 65% Wt, 1 Eq, 90.5 μmol), formaldehyde solution in H₂O (44 mg, 40 μL, 37% Wt, 5.9 Eq, 0.54 mmol) and DIPEA (111 mg, 150 μL, 9.52 Eq, 861 μmol) in CHCl₃ (3.0 mL) was stirred at rt for 1 h. NaBH(OAc)₃ (101.4 mg, 97% Wt, 5.13 Eq, 464.1 μmol) was added and the reaction mixture was stirred at rt overnight. The reaction mixture was diluted with EtOAc (5 mL), washed with sat. NaHCO₃ (5 mL) and brine (5 mL), dried (Na₂SO₄), filtered and concentrated in vacuo. The crude product was purified by chromatography on silica gel (4 g cartridge, 0-10% MeOH/DCM) to afford the sub-title compound (JJ-2) (46.0 mg, 67 μmol, 75%, 95% Purity) as a yellow oil. m/z 648.2 (M+H)⁺ (ES+). ¹H NMR (400 MHz, MeOD) δ 8.37 (s, 1H), 8.18 (dt, J=7.9, 1.4 Hz, 1H), 8.07 (t, J=1.8 Hz, 1H), 7.77 (dd, J=8.6, 5.4 Hz, 1H), 7.65 (dd, J=5.5, 1.6 Hz, 1H), 7.57-7.48 (m, 2H), 7.45 (dd, J=8.8, 2.8 Hz, 1H), 7.39 (dt, J=7.9, 1.4 Hz, 1H), 4.41-4.35 (m, 1H), 3.90 (d, 1H), 3.88 (d, J=14.6 Hz, 1H), 3.19 (s, J=14.6 Hz, 3H), 2.73-2.63 (m, 1H), 2.31 (s, 3H), 1.94-1.84 (m, 3H), 1.82-1.73 (m, 1H), 1.72-1.55 (m, 2H), 0.93 (s, 9H), 0.11 (s, 3H), 0.09 (s, 3H).

Step 3: (1S,2R)-2-(((6,7-Difluoro-2-(4′-fluoro-2′-(4-methyl-4H-1,2,4-triazol-3-yl)-[1,1′-biphenyl]-3-yl)benzo[d]oxazol-5-yl)methyl)(methyl)amino)cyclopentan-1-ol (JJ-3). To a stirred solution of the product from step 2 above (JJ-2) (46.0 mg, 95% Wt, 1 Eq, 67.5 μmol) in THF (1.0 mL) at rt was added TBAF in THF (363 mg, 400 μL, 1.0 molar, 5.93 Eq, 400 μmol). The reaction mixture was stirred at rt overnight. The volatiles were removed in vacuo. The crude product was purified by chromatography on silica gel (4 g cartridge, 0-10% MeOH/DCM) to afford the title compound (JJ-3) (26.8 mg, 50 μmol, 74%, 99% Purity) as an off-white solid. m/z 534.1 (M+H)⁺ (ES+). ¹H NMR (400 MHz, MeOD) δ 8.38 (s, 1H), 8.20 (d, J=7.9 Hz, 1H), 8.09-8.06 (m, 1H), 7.78 (dd, J=8.7, 5.4 Hz, 1H), 7.66 (d, J=5.4 Hz, 1H), 7.59-7.51 (m, 2H), 7.46 (dd, J=8.7, 2.8 Hz, 1H), 7.44-7.39 (m, 1H), 4.27-4.23 (m, 1H), 3.89 (d, J=13.7 Hz, 1H), 3.82 (d, J=13.5 Hz, 1H), 3.20 (s, 3H), 2.67-2.59 (m, 1H), 2.27 (s, 3H), 2.00-1.88 (m, 2H), 1.88-1.75 (m, 3H), 1.75-1.63 (m, 1H). 1× Exchangeable OH proton not observed.

### Example 381: 1-(2-(4′-Fluoro-2′-(4-methyl-4H-1,2,4-triazol-3-yl)-[1,1′-biphenyl]-3-yl)-7-methylbenzo[d]oxazol-5-yl)-2-methoxy-N-(2-methoxyethyl)ethan-1-amine (JK-7)

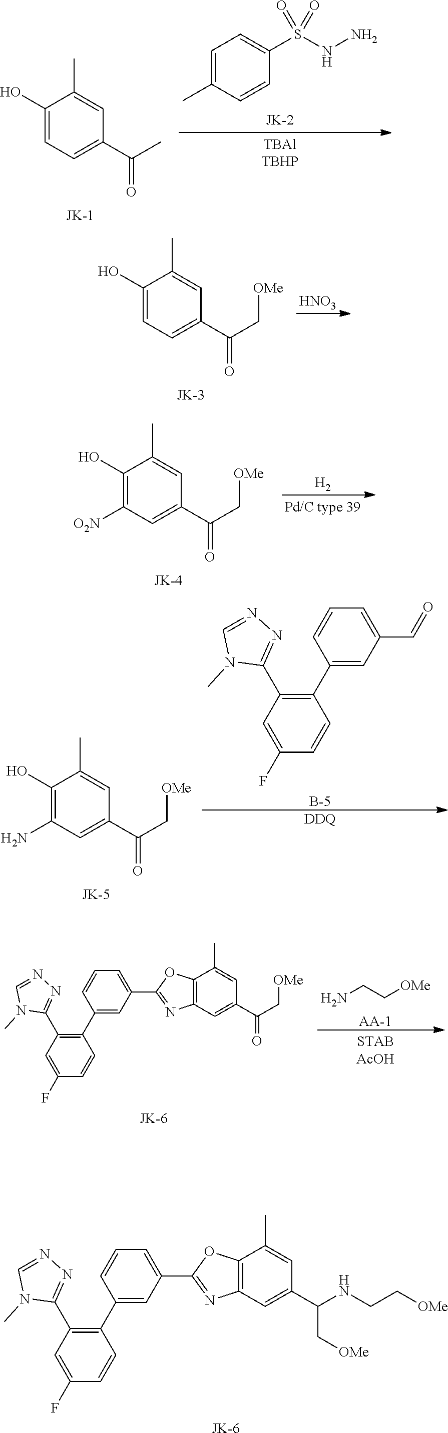

Step 1: 1-(4-Hydroxy-3-methylphenyl)-2-methoxyethan-1-one (JK-3). To a stirred solution of 1-(4-hydroxy-3-methylphenyl)ethan-1-one (JK-1) (1.0171 g, 1 Eq, 6.7725 mmol) in dry MeOH (20 mL) at rt was added 4-methylbenzenesulfonic acid hydrazide (JK-2) (1.3 g, 97% Wt, 1.0 Eq, 6.8 mmol), TBAI (493.8 mg, 0.1974 Eq, 1.337 mmol) and tert-Butyl hydroperoxide (5.4 g, 6.0 mL, 70% Wt, 6.2 Eq, 42 mmol). The reaction mixture was stirred at rt overnight. The reaction mixture was diluted with MTBE (50 mL) and washed with sat. Na₂S₂O₃ (50 mL). The aqueous layer was extracted with MTBE (3×50 mL). The combined organic layers were collected, dried (Na₂SO₄), filtered and concentrated in vacuo. The crude product was purified by chromatography on silica gel (40 g cartridge, 0-100% EtOAc/isohexane) to afford the sub-title compound (JK-3) (789.3 mg, 2.7 mmol, 40%, 62% Purity) as a yellow solid, along with recovery of unreacted starting material 1-(4-hydroxy-3-methylphenyl)ethan-1-one (JK-1) (458.0 mg, 2.1 mmol, 31%, 68% Purity) as a pale yellow solid. m/z 181.6 (M+H)⁺ (ES+). ¹H NMR (400 MHz, DMSO) δ 10.30 (s, 1H), 7.69 (d, J=2.2 Hz, 1H), 7.64 (dd, J=8.4, 2.3 Hz, 1H), 6.85 (d, J=8.4 Hz, 1H), 4.65 (s, 2H), 3.33 (s, 3H), 2.15 (s, 3H).

Step 2: 1-(4-Hydroxy-3-methyl-5-nitrophenyl)-2-methoxyethan-1-one (JK-4). To a stirred solution of the product from step 1 above (JK-3) (789.3 mg, 62% Wt, 1 Eq, 2.716 mmol) in AcOH (5.0 mL) at rt was added nitric acid (423 mg, 300 μL, 2.47 Eq, 6.71 mmol). The reaction mixture was stirred at rt overnight. The mixture was poured into ice water (10 mL) and filtered. The filtrate was extracted with EtOAc (3×10 mL). The combined organic layers were collected, dried (Na₂SO₄), filtered and concentrated in vacuo. The crude product was purified by chromatography on silica gel (40 g cartridge, 0-100% EtOAc/isohexane) to afford the sub-title compound (JK-4) (382.2 mg, 1.2 mmol, 44%, 71% Purity) as a yellow solid. ¹H NMR (400 MHz, DMSO) δ 8.34 (d, J=2.3 Hz, 1H), 8.05 (dd, J=2.3, 0.9 Hz, 1H), 4.76 (s, 2H), 3.35 (s, 3H), 2.30 (s, 3H). 1× Exchangeable OH proton not observed.

Step 3: 1-(3-Amino-4-hydroxy-5-methylphenyl)-2-methoxyethan-1-one (JK-5). To a solution of the product from step 2 above (JK-4) (382.2 mg, 71% Wt, 1 Eq, 1.205 mmol) in MeOH (10 mL) was added Pd/C 87 L (175.1 mg, 5% Wt, 0.06827 Eq, 82.27 μmol). The reaction mixture was stirred at rt with 5 atm of H₂ for 3 h. Pd/C Type 39 (93.3 mg, 10% Wt, 0.0728 Eq, 87.7 μmol) was added and the reaction mixture was stirred at rt with 5 atm of H₂ overnight. The mixture was filtered and the volatiles were removed in vacuo to afford the sub-title compound (JK-5) (377.3 mg, 1.1 mmol, 88%, 55% Purity) as an orange oil. m/z 196.3 (M+H)⁺ (ES+). ¹H NMR (400 MHz, DMSO) δ 7.08 (d, J=2.3 Hz, 1H), 7.01 (d, J=2.3 Hz, 1H), 4.60 (s, 2H), 4.09 (br s, 3H), 3.33 (s, 3H), 2.15 (s, 3H).

Step 4: 1-(2-(4′-Fluoro-2′-(4-methyl-4H-1,2,4-triazol-3-yl)-[1,1′-biphenyl]-3-yl)-7-methylbenzo[d]oxazol-5-yl)-2-methoxyethan-1-one (JK-6). A solution of intermediate (B-5) (254.3 mg, 97% Wt, 1 Eq, 876.9 μmol) and the product from step 3 above (JK-5) (377.3 mg, 58% Wt, 1.278 Eq, 1.121 mmol) in MeOH (10 mL) was heated at 45° C. overnight. The reaction mixture was cooled to rt and the volatiles were removed in vacuo. The residue was redissolved in DCM (10 mL). DDQ (260.3 mg, 1.31 Eq, 1.15 mmol) was added and the resulting mixture was stirred at rt for 2 h. The reaction mixture was diluted with DCM (10 mL), washed with sat. NaHCO₃ (10 mL) and brine (10 mL). The organic layers were collected, dried (Na₂SO₄), filtered and concentrated in vacuo. The crude product was purified by chromatography on silica gel (24 g cartridge, 0-10% MeOH/DCM) to afford the sub-title compound (JK-6) (196.1 mg, 0.26 mmol, 30%, 61% Purity) as a brown solid. m/z 457.1 (M+H)⁺ (ES+). ¹H NMR (400 MHz, DMSO) δ 8.43 (s, 1H), 8.21 (d, J=1.6 Hz, 1H), 8.17 (dt, J=7.9, 1.4 Hz, 1H), 7.95 (t, J=1.8 Hz, 1H), 7.86 (t, J=1.3 Hz, 1H), 7.83-7.75 (m, 1H), 7.68-7.50 (m, 3H), 7.45-7.35 (m, 1H), 4.87 (s, 2H), 3.39 (s, 3H), 3.17 (s, 3H), 2.62 (s, 3H).

Step 5: 1-(2-(4′-Fluoro-2′-(4-methyl-4H-1,2,4-triazol-3-yl)-[1,1′-biphenyl]-3-yl)-7-methylbenzo[d]oxazol-5-yl)-2-methoxy-N-(2-methoxyethyl)ethan-1-amine (JK-7). To a stirred solution of the product from step 4 above (JK-6) (30.9 mg, 61% Wt, 1 Eq, 41.3 μmol) and 1-amino-2-methoxyethane (AA-1) (13.0 mg, 15.0 μL, 4.19 Eq, 173 μmol) in dry DCE (2.0 mL) under a nitrogen atmosphere at rt was added acetic acid (15.7 mg, 15.0 μL, 6.35 Eq, 262 μmol). The reaction mixture was stirred at rt for 1 h. NaBH(OAc)₃ (50.5 mg, 97% Wt, 5.60 Eq, 231 μmol) was added and the resulting mixture was stirred at rt for 4 h. The reaction mixture was heated at 60° C. overnight. 1-Amino-2-methoxyethane (AA-1) (13.0 mg, 15.0 μL, 4.19 Eq, 173 μmol) was added and the reaction mixture was heated at 60° C. for 5 h. Further 1-Amino-2-methoxyethane (AA-1) (13.0 mg, 15.0 μL, 4.19 Eq, 173 μmol) was added and the reaction was continued to be heated at 60° C. for 2 h. The reaction mixture was cooled to rt and was left stirring at rt overnight. The reaction mixture was diluted with EtOAc (5 mL), washed with sat. NaHCO₃ (5 mL) and brine (5 mL), dried (Na₂SO₄), filtered and concentrated in vacuo. The crude product was purified by chromatography on silica gel (4 g cartridge, 0-10% MeOH/DCM) to afford product with ˜52% purity. The crude product was purified by preparative HPLC (Waters, basic (0.3% ammonia), basic, Waters XBridge BEH C18 ODB, 5 μm, 30×100 mm column, 42.5-100% MeCN in water) to afford the title compound (JK-7) (1.2 mg, 2.3 μmol, 5.6%, 99% Purity) as an off-white solid. m/z 516.2 (M+H)⁺ (ES+). ¹H NMR (400 MHz, MeOD) δ 8.38 (s, 1H), 8.22-8.17 (m, 1H), 8.05 (t, J=1.8 Hz, 1H), 7.79 (dd, J=8.7, 5.4 Hz, 1H), 7.59-7.51 (m, 3H), 7.46 (dd, J=8.8, 2.7 Hz, 1H), 7.44-7.39 (m, 1H), 7.27-7.24 (m, 1H), 4.01 (t, J=6.4 Hz, 1H), 3.52 (d, J=6.3 Hz, 2H), 3.50-3.41 (m, 2H), 3.38 (s, 3H), 3.35 (s, 3H), 3.18 (s, 3H), 2.66 (ddd, J=11.4, 7.1, 4.1 Hz, 1H), 2.60 (s, 3H), 2.59-2.54 (m, 1H). 1× Exchangeable NH proton not observed.

### Example 382: 1-(((2-(5-Cyclopropyl-4′-fluoro-2′-(4-methyl-4H-1,2,4-triazol-3-yl)-[1,1′-biphenyl]-3-yl)-7-(trifluoromethyl)benzo[d]oxazol-5-yl)methyl)amino)-2-methylpropan-2-ol (JL-1)

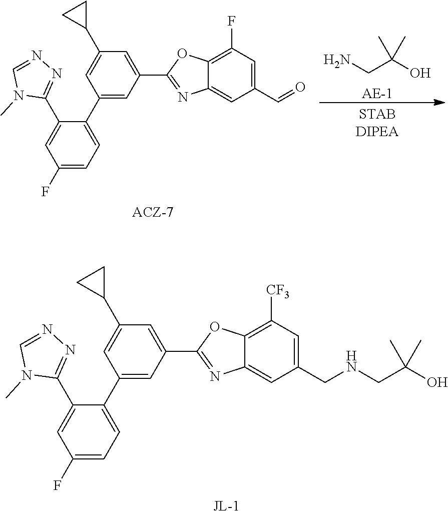

A solution of intermediate (ACZ-7) (38.9 mg, 98% Wt, 1 Eq, 75.3 μmol), 1-amino-2-methylpropan-2-ol (AE-1) (14 mg, 15 μL, 2.1 Eq, 0.16 mmol) and DIPEA (52 mg, 70 μL, 5.3 Eq, 0.40 mmol) in CHCl₃ (2.0 mL) was stirred at rt for 1 h. NaBH(OAc)₃ (85.8 mg, 97% Wt, 5.22 Eq, 393 μmol) was added and the reaction mixture was stirred at rt overnight. The reaction mixture was diluted with EtOAc (5 mL), washed with sat. NaHCO₃ (5 mL) and brine (5 mL), dried (Na₂SO₄), filtered and concentrated in vacuo. The crude product was purified by chromatography on silica gel (4 g cartridge, 0-10% MeOH/DCM) to afford the title compound (JL-1) (17.4 mg, 30 μmol, 40%) as a white solid. m/z 580.2 (M+H)⁺ (ES+). ¹H NMR (400 MHz, DMSO) δ 8.43 (s, 1H), 8.07 (s, 1H), 7.92 (s, 1H), 7.82-7.76 (m, 2H), 7.73 (s, 1H), 7.63-7.56 (m, 1H), 7.53 (dd, J=9.1, 2.8 Hz, 1H), 6.86 (s, 1H), 4.21 (s, 1H), 3.93 (s, 2H), 3.15 (s, 3H), 2.38 (s, 2H), 2.04 (d, J=5.0 Hz, 1H), 1.11 (s, 6H), 0.98 (q, J=5.8 Hz, 2H), 0.59 (d, J=5.8 Hz, 2H).

### Example 383: (1S,2R)-2-(((2-(5-Cyclopropyl-4′-fluoro-2′-(4-methyl-4H-1,2,4-triazol-3-yl)-[1,1′-biphenyl]-3-yl)-7-fluorobenzo[d]oxazol-5-yl)methyl)amino)cyclopentan-1-ol (JM-4)

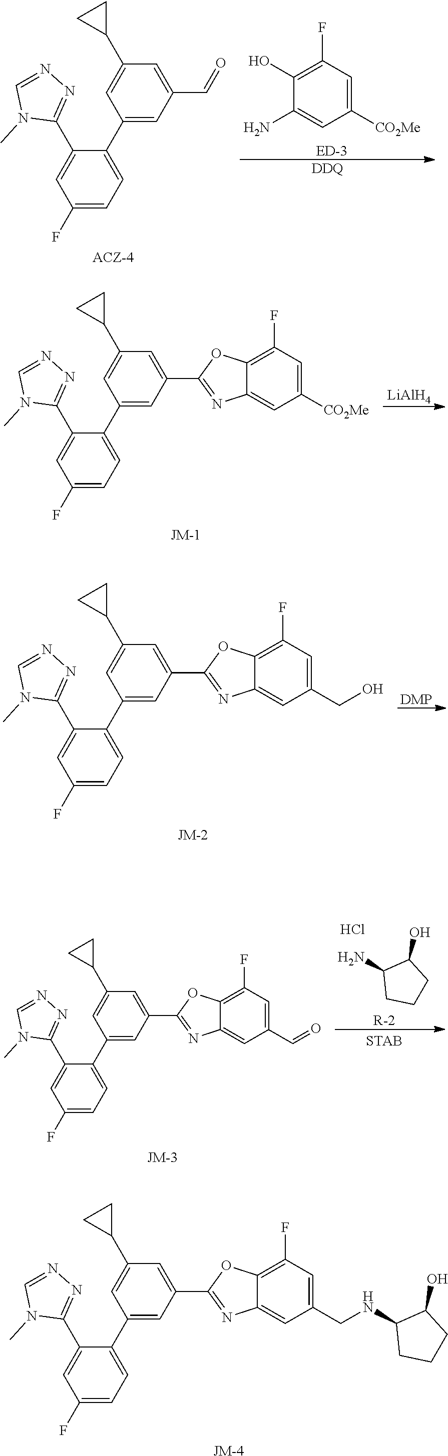

Step 1: Methyl 2-(5-cyclopropyl-4′-fluoro-2′-(4-methyl-4H-1,2,4-triazol-3-yl)-[1,1′-biphenyl]-3-yl)-7-fluorobenzo[d]oxazole-5-carboxylate (JM-1). A solution of intermediate (ACZ-4) (214 mg, 98% Wt, 1 Eq, 653 μmol) and intermediate (ED-3) (133 mg, 1.1 Eq, 718 μmol) in EtOH (5.00 mL) was heated to 80° C. under N₂ overnight. The reaction mixture was concentrated in vacuo and the residue was dissolved in DCM (5 mL). DDQ (163 mg, 1.1 Eq, 718 μmol) was added to the reaction mixture and stirred at rt for 1 h. The reaction was quenched with NaHCO₃ and extracted with DCM (3×10 mL). The organic extracts were combined, dried (phase separator), and concentrated in vacuo. The crude product was purified by chromatography on silica gel (12 g cartridge, 0-10% MeOH/DCM) to afford the sub-title compound (JM-1) (96 mg, 197 μmol, 29%, 98% purity) as a dark brown solid. m/z 487.1 (M+H)⁺ (ES+)

Step 2: (2-(5-Cyclopropyl-4′-fluoro-2′-(4-methyl-4H-1,2,4-triazol-3-yl)-[1,1′-biphenyl]-3-yl)-7-fluorobenzo[d]oxazol-5-yl)methanol (JM-2). To a solution of the product from step 1 above (JM-1) (96.0 mg, 98% Wt, 1 Eq, 193 μmol) in THF (2 mL) was added LiAlH₄ (8.07 mg, 213 μL, 1 molar, 1.1 Eq, 213 μmol) dropwise at 0° C. This mixture was stirred 2 h at this temperature. The reaction mixture was diluted with distilled water (10 mL) and acidified with 2 mL 1M HCl and transferred into a separating funnel. The layer was extracted with DCM (3×10 mL). The combined extracted layers were collected, dried (Na₂SO₄), filtered and concentrated in vacuo. The crude product was taken for follow-up oxidation step without further purification. m/z 459.3 (M+H)⁺ (ES+).

Step 3: 2-(5-Cyclopropyl-4′-fluoro-2′-(4-methyl-4H-1,2,4-triazol-3-yl)-[1,1′-biphenyl]-3-yl)-7-fluorobenzo[d]oxazole-5-carbaldehyde (JM-3). To a stirred solution of the product from step 2 above (JM-2) (85.0 mg, 94% Wt, 1 Eq, 174 μmol) in dry DCM (2 mL) under a nitrogen atmosphere at 25° C. was added DMP (148 mg, 2 Eq, 349 μmol). The reaction mixture was stirred for 3 hours. The reaction mixture was diluted with DCM (10 mL) and washed with sat. aq. NaHCO₃ (10 mL) and brine. Organics were separated, dried (MgSO₄), filtered and evaporated in vacuo. The crude product was purified by chromatography on silica gel (4 g cartridge, 0-10% MeOH/DCM) to afford the sub-title compound (JM-3) (77.0 mg, 0.17 mmol, 95%, 98% Purity) as a clear colourless solid. m/z 457.1 (M+H)⁺ (ES+)

Step 4: (1S,2R)-2-(((2-(5-Cyclopropyl-4′-fluoro-2′-(4-methyl-4H-1,2,4-triazol-3-yl)-[1,1′-biphenyl]-3-yl)-7-fluorobenzo[d]oxazol-5-yl)methyl)amino)cyclopentan-1-ol (JM-4). To a stirred solution of the product from step 3 above (JM-3) (20.0 mg, 98% Wt, 1 Eq, 42.9 μmol) in dry CHCl₃ (2.00 mL) under a nitrogen atmosphere at 40° C. was added cis-2-amino-cyclopentanol, HCl (R-2) (11.8 mg, 2 Eq, 85.9 μmol). The reaction mixture was stirred for 12 hours at 40° C. NaBH(OAc)₃ (36.4 mg, 4 Eq, 172 μmol) was added and continued the stirring at 40° C. for 2 hour. The reaction mixture was diluted with sat NaHCO₃ (2 mL) and transferred into a separating funnel. The layer was extracted with DCM (3×5 mL). The combined extracted layers were collected, dried (Na₂SO₄), filtered and concentrated in vacuo. The crude product was purified by chromatography on silica gel (4 g cartridge, 0-10% (0.7 M ammonia/MeOH)/DCM) to afford the title compound (JM-4) (17.0 mg, 31 μmol, 72%, 98% Purity) as a clear white solid. m/z 542.2 (M+H)⁺ (ES+). ¹H NMR (400 MHz, MeOD) δ 8.43 (s, 1H), 7.98 (dt, J=11.9, 1.7 Hz, 2H), 7.80 (dd, J=8.6, 5.5 Hz, 1H), 7.64 (d, J=1.3 Hz, 1H), 7.56 (td, J=8.5, 2.8 Hz, 1H), 7.47 (dd, J=8.8, 2.7 Hz, 1H), 7.35 (dd, J=11.0, 1.4 Hz, 1H), 6.98 (t, J=1.8 Hz, 1H), 4.22 (td, J=4.4, 2.2 Hz, 1H), 4.10-3.93 (m, 2H), 3.18 (s, 3H), 3.06 (dt, J=8.0, 4.3 Hz, 1H), 2.04-1.98 (m, 1H), 1.86 (dt, J=13.1, 6.2 Hz, 2H), 1.68-1.54 (m, 2H), 1.37 (dd, J=6.9, 5.2 Hz, 2H), 1.10-1.02 (m, 2H), 0.66 (dt, J=6.7, 4.7 Hz, 2H). Exchangeable NH & OH not observed.

### Example 384: 1-(((7-Fluoro-2-(4′-fluoro-2′-(4-methyl-4H-1,2,4-triazol-3-yl)-[1,1′-biphenyl]-3-yl)benzo[d]oxazol-5-yl)methyl)amino)-2-methylpropan-2-ol (JN-1)

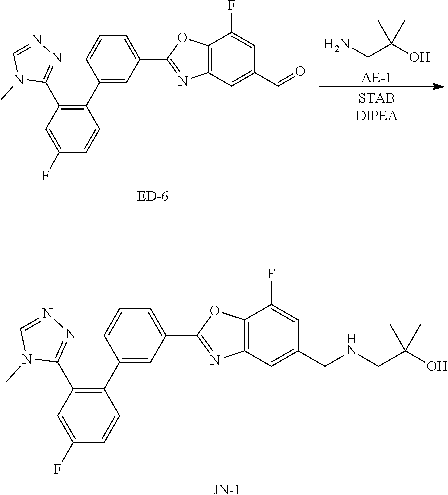

A solution of intermediate (ED-6) (30.0 mg, 76% Wt, 1 Eq, 54.8 μmol), 1-amino-2-methylpropan-2-ol (AE-1) (9.76 mg, 2 Eq, 110 μmol) and DIPEA (21.2 mg, 28.6 μL, 3 Eq, 164 μmol) in CHCl₃ (2 mL) was stirred for 12 hour at 40° C. NaBH(OAc)₃ (46.4 mg, 4 Eq, 219 μmol) was added and stirring was continued at 40° C. for another 2 hour. The reaction mixture was diluted with NaHCO₃ (25 mL) and transferred into a separating funnel. The layer was extracted with EtOAc (3×25 mL). The combined extracted layers were collected, dried (Na₂SO₄), filtered and concentrated in vacuo. The crude product was purified by chromatography on silica gel (4 g cartridge, 0-10% MeOH/DCM) to afford the title compound (JN-1) (18.0 mg, 36 μmol, 66%, 99% Purity) as a clear white powder. m/z 490.1 (M+H)⁺ (ES+). ¹H NMR (400 MHz, MeOD) δ 8.41 (s, 1H), 8.24 (dt, J=8.0, 1.3 Hz, 1H), 8.13 (t, J=1.9 Hz, 1H), 7.81 (dd, J=8.6, 5.5 Hz, 1H), 7.64-7.54 (m, 3H), 7.52-7.41 (m, 2H), 7.33 (dd, J=11.0, 1.4 Hz, 1H), 3.97 (s, 2H), 3.23 (s, 3H), 2.59 (s, 2H), 1.25 (s, 6H). Exchangeable NH & OH not observed.

### Example 385: 1-Cyclobutyl-N-((2-(5-cyclopropyl-4′-fluoro-2′-(4-methyl-4H-1,2,4-triazol-3-yl)-[1,1′-biphenyl]-3-yl)-7-fluorobenzo[d]oxazol-5-yl)methyl)methanamine (JO-1)

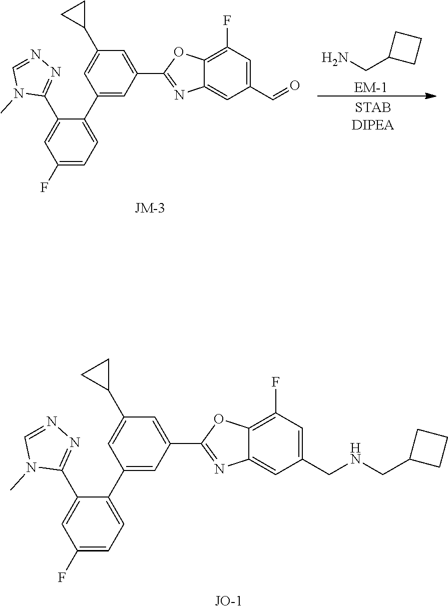

A solution of intermediate (JM-3) (20.0 mg, 76% Wt, 1 Eq, 33.3 μmol), (cyclobutylmethyl)amine (EM-1) (5.67 mg, 2 Eq, 66.6 μmol) and DIPEA (12.9 mg, 17.4 μL, 3 Eq, 99.9 μmol) in CHCl₃ (2 mL) was stirred for 12 hour at 40° C. NaBH(OAc)₃ (28.2 mg, 4 Eq, 133 μmol) was added and stirring was continued at 40° C. for another 2 hour. The reaction mixture was diluted with NaHCO₃ (25 mL) and transferred into a separating funnel. The layer was extracted with EtOAc (3×25 mL). The combined extracted layers were collected, dried (Na₂SO₄), filtered and concentrated in vacuo. The crude product was purified by chromatography on silica gel (4 g cartridge, 0-10% MeOH/DCM) to afford the title compound (JO-1) (12.0 mg, 23 μmol, 68%, 99% Purity) as a clear white powder. m/z 526.2 (M+H)⁺ (ES+). ¹H NMR (400 MHz, MeOD) δ 8.43 (s, 1H), 7.99 (dt, J=10.3, 1.7 Hz, 2H), 7.80 (dd, J=8.7, 5.5 Hz, 1H), 7.59 (d, J=1.2 Hz, 1H), 7.55 (dd, J=8.5, 2.8 Hz, 1H), 7.47 (dd, J=8.8, 2.8 Hz, 1H), 7.30 (dd, J=11.0, 1.4 Hz, 1H), 6.97 (t, J=1.7 Hz, 1H), 3.90 (s, 2H), 3.17 (s, 3H), 2.67 (d, J=7.2 Hz, 2H), 2.57 (dt, J=15.1, 7.6 Hz, 1H), 2.21-2.06 (m, 2H), 2.03-1.93 (m, 2H), 1.93-1.82 (m, 1H), 1.79-1.66 (m, 2H), 1.13-1.00 (m, 2H), 0.66 (dt, J=6.7, 4.7 Hz, 2H). Exchangeable NH not observed.

### Example 386: (S)-1-(((7-Fluoro-2-(4′-fluoro-2′-(4-methyl-4H-1,2,4-triazol-3-yl)-[1,1′-biphenyl]-3-yl)benzo[d]oxazol-5-yl)methyl)amino)propan-2-ol (JP-1)

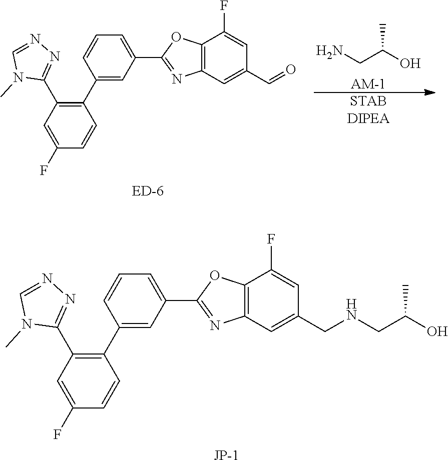

A solution of intermediate (ED-6) (30.0 mg, 93% Wt, 1 Eq, 67.0 μmol), (s)-1-aminopropan-2-ol (AM-1) (10.1 mg, 10.6 μL, 2 Eq, 134 μmol) and DIPEA (26.0 mg, 35.0 μL, 3 Eq, 201 μmol) in CHCl₃ (2 mL) was stirred for 48 hour at 40° C. NaBH(OAc)₃ (56.8 mg, 4 Eq, 268 μmol) was added and stirring was continued at 40° C. for another 2 hour. The reaction mixture was diluted with NaHCO₃ (25 mL) and transferred into a separating funnel. The layer was extracted with EtOAc (3×25 mL). The combined extracted layers were collected, dried (Na₂SO₄), filtered and concentrated in vacuo. The crude product was purified by chromatography on silica gel (4 g cartridge, 0-10% MeOH/DCM) to afford the title compound (JP-1) (15.0 mg, 31 μmol, 47%, 99% Purity) as a clear white powder. m/z 476.2 (M+H)⁺ (ES+). ¹H NMR (400 MHz, MeOD) δ 8.40 (s, 1H), 8.29-8.22 (m, 1H), 8.11 (t, J=1.9 Hz, 1H), 7.80 (dd, J=8.6, 5.4 Hz, 1H), 7.71 (d, J=1.4 Hz, 1H), 7.65-7.53 (m, 2H), 7.49 (dt, J=9.0, 2.6 Hz, 2H), 7.41 (dd, J=10.8, 1.4 Hz, 1H), 4.24 (s, 2H), 4.03 (ddd, J=9.5, 6.3, 3.3 Hz, 1H), 3.23 (s, 3H), 2.96 (dd, J=12.4, 3.3 Hz, 1H), 2.80 (dd, J=12.4, 9.4 Hz, 1H), 1.24 (d, J=6.3 Hz, 3H). Exchangeable NH & OH not observed.

### Example 387: 1-(((2-(5-Cyclopropyl-4′-fluoro-2′-(4-methyl-4H-1,2,4-triazol-3-yl)-[1,1′-biphenyl]-3-yl)-7-fluorobenzo[d]oxazol-5-yl)methyl)amino)-2-methylpropan-2-ol (JQ-1)

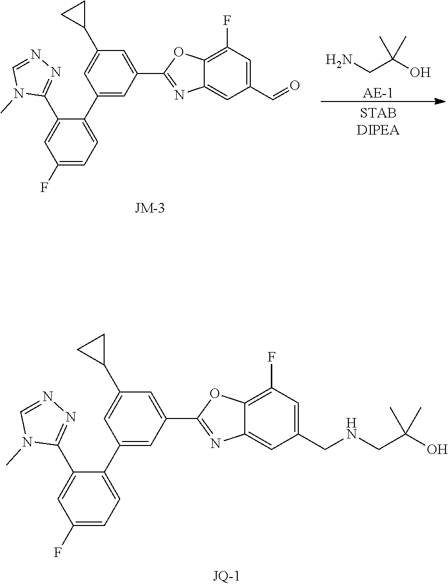

A solution of intermediate (JQ-1) (20.0 mg, 76% Wt, 1 Eq, 33.3 μmol), 1-amino-2-methylpropan-2-ol (AE-1) (5.94 mg, 2 Eq, 66.6 μmol) and DIPEA (12.9 mg, 17.4 μL, 3 Eq, 99.9 μmol) in CHCl₃ (2 mL) was stirred for 12 hour at 40° C. NaBH(OAc)₃ (28.2 mg, 4 Eq, 133 μmol) was added and stirring was continued at 40° C. for another 2 hour. The reaction mixture was diluted with NaHCO₃ (25 mL) and transferred into a separating funnel. The layer was extracted with EtOAc (3×25 mL). The combined extracted layers were collected, dried (Na₂SO₄), filtered and concentrated in vacuo. The crude product was purified by chromatography on silica gel (4 g cartridge, 0-10% MeOH/DCM) to afford the title compound (JQ-1) (15.0 mg, 28 μmol, 84%, 99% Purity) as a clear white powder. m/z 530.2.1 (M+H)⁺ (ES+). ¹H NMR (400 MHz, MeOD) δ 8.43 (s, 1H), 7.98 (dt, J=9.3, 1.7 Hz, 2H), 7.80 (dd, J=8.6, 5.4 Hz, 1H), 7.59 (d, J=1.3 Hz, 1H), 7.58-7.53 (m, 1H), 7.47 (dd, J=8.8, 2.8 Hz, 1H), 7.32 (dd, J=11.1, 1.3 Hz, 1H), 6.96 (t, J=1.7 Hz, 1H), 3.96 (s, 2H), 3.17 (s, 3H), 2.58 (s, 2H), 2.07-1.94 (m, 1H), 1.25 (s, 6H), 1.11-0.99 (m, 2H), 0.66 (dt, J=6.7, 4.7 Hz, 2H). Exchangeable NH & OH not observed.

### Example 388: (R)-2-(4′-Fluoro-2′-(4-methyl-4H-1,2,4-triazol-3-yl)-[1,1′-biphenyl]-3-yl)-5-((3-methoxypiperidin-1-yl)methyl)-7-(trifluoromethyl)benzo[d]oxazole (JR-2)

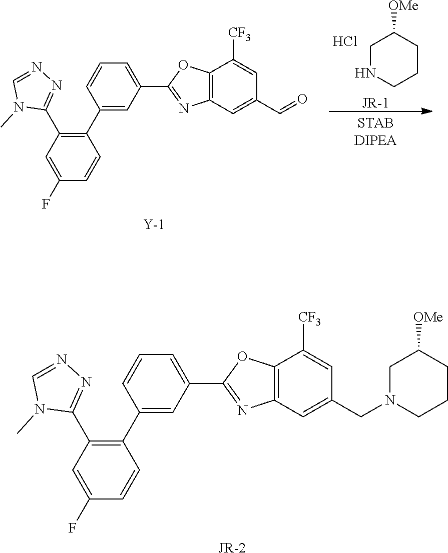

(R)-3-methoxypiperidine, HCl (JR-1) (18.3 mg, 2 Eq, 121 μmol) was added to a stirred mixture of DIPEA (23.4 mg, 31.6 μL, 3 Eq, 181 μmol), intermediate (Y-1) (30.0 mg, 94% Wt, 1 Eq, 60.5 μmol) and NaBH(OAc)₃ (51.3 mg, 4 Eq, 242 μmol) in CHCl₃ (2.00 mL). The reaction mixture was stirred at 40° C. overnight and then at rt. The reaction mixture was diluted with DCM (4 mL) and washed with sat. aq. NaHCO₃ (10 mL). The organic phase was separated and evaporated to afford the crude product. The crude product was purified by preparative HPLC (Waters, Basic (0.3% ammonium bicarbonate), Basic Waters XBridge BEH C18 ODB, 5 μm, 30×100 mm column 45-100% MeCN in water, to afford the title compound (JR-2) (9.00 mg, 16 μmol, 26%, 98% Purity) as a colourless foam. m/z 566.4 (M+H)⁺ (ES+). ¹H NMR (400 MHz, MeOD) δ 8.41 (s, 1H), 8.27-8.21 (m, 1H), 8.09 (t, J=1.8 Hz, 1H), 8.00 (s, 1H), 7.82 (dd, J=8.7, 5.4 Hz, 1H), 7.76 (s, 1H), 7.65-7.53 (m, 2H), 7.53-7.44 (m, 2H), 3.80-3.71 (m, 2H), 3.42-3.36 (m, 1H), 3.28 (s, 3H), 2.93-2.86 (m, 1H), 2.71-2.62 (m, 1H), 2.29-2.12 (m, 2H), 2.01-1.91 (m, 1H), 1.86-1.76 (m, 1H), 1.62-1.54 (m, 1H), 1.38-1.29 (m, 1H). Three missing protons under solvent peak.

### Example 389: 1-((((2-(5-Cyclopropyl-4′-fluoro-2′-(4-methyl-4H-1,2,4-triazol-3-yl)-[1,1′-biphenyl]-3-yl)-7-(trifluoromethyl)benzo[d]oxazol-5-yl)methyl)amino)methyl)cyclobutan-1-ol (JS-1)

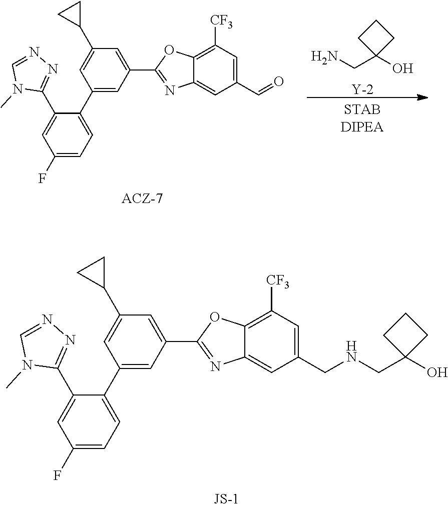

A solution of intermediate (ACZ-7) (20.0 mg, 98% Wt, 1 Eq, 38.7 μmol), 1-aminomethyl-cyclobutanol (Y-2) (7.83 mg, 2 Eq, 77.4 μmol) and DIPEA (15.0 mg, 20.2 μL, 3 Eq, 116 μmol) in CHCl₃ (2 mL) was stirred for 12 hour at 40° C. NaBH(OAc)₃ (32.8 mg, 4 Eq, 155 μmol) was added and stirring was continued at 40° C. for another 2 hour. The reaction mixture was diluted with NaHCO₃ (5 mL) and transferred into a separating funnel. The layer was extracted with DCM (3×5 mL). The combined extracted layers were collected, dried (Na₂SO₄), filtered and concentrated in vacuo. The crude product was purified by chromatography on silica gel (4 g cartridge, 0-10% MeOH/DCM) to afford the title compound (JS-1) (12.0 mg, 20 μmol, 52%, 99% Purity) as a clear white powder. m/z 592.2 (M+H)⁺ (ES+). ¹H NMR (400 MHz, MeOD) δ 8.43 (s, 1H), 8.07-7.99 (m, 2H), 7.94 (t, J=1.7 Hz, 1H), 7.84-7.76 (m, 2H), 7.57 (td, J=8.5, 2.8 Hz, 1H), 7.48 (dd, J=8.8, 2.8 Hz, 1H), 6.99 (t, J=1.8 Hz, 1H), 4.05 (s, 2H), 3.21 (s, 3H), 2.75 (s, 2H), 2.23-1.97 (m, 6H), 1.55 (dt, J=11.4, 8.9 Hz, 1H), 1.12-1.01 (m, 2H), 0.66 (dt, J=6.7, 4.7 Hz, 2H). Exchangeable NH & OH not observed.

### Example 390: N-((2-(5-Cyclopropyl-4′-fluoro-2′-(4-methyl-4H-1,2,4-triazol-3-yl)-[1,1′-biphenyl]-3-yl)-7-(trifluoromethyl)benzo[d]oxazol-5-yl)methyl)-2-methoxyethan-1-amine (JT-1)

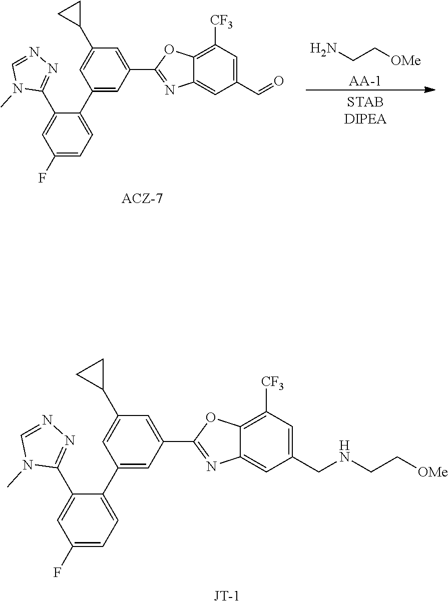

A solution of intermediate (ACZ-7) (20.0 mg, 98% Wt, 1 Eq, 38.7 μmol), 1-amino-2-methoxyethane (AA-1) (2.91 mg, 3.36 μL, 1 Eq, 38.7 μmol) and DIPEA (15.0 mg, 20.2 μL, 3 Eq, 116 μmol) in CHCl₃ (2 mL) was stirred for 12 hour at 40° C. NaBH(OAc)₃ (32.8 mg, 4 Eq, 155 μmol) was added and stirring was continued at 40° C. for another 5 hour. The reaction mixture was diluted with NaHCO₃ (5 mL) and transferred into a separating funnel. The layer was extracted with DCM (3×25 mL). The combined extracted layers were collected, dried (Na₂SO₄), filtered and concentrated in vacuo. The crude product was purified by chromatography on silica gel (4 g cartridge, 0-10% MeOH/DCM) to afford the title compound (JT-1) (15.0 mg, 26 μmol, 68%, 99% Purity) as a clear white powder. m/z 566.1 (M+H)⁺ (ES+). ¹H NMR (400 MHz, MeOD) δ 8.43 (s, 1H), 8.05-7.98 (m, 2H), 7.94 (t, J=1.7 Hz, 1H), 7.84-7.75 (m, 2H), 7.57 (td, J=8.4, 2.7 Hz, 1H), 7.48 (dd, J=8.8, 2.7 Hz, 1H), 6.99 (t, J=1.7 Hz, 1H), 4.01 (s, 2H), 3.57 (t, J=5.3 Hz, 2H), 3.39 (s, 3H), 3.21 (s, 3H), 2.82 (t, J=5.3 Hz, 2H), 2.02 (ddd, J=13.3, 8.6, 5.0 Hz, 1H), 1.11-1.02 (m, 2H), 0.66 (dt, J=6.6, 4.7 Hz, 2H). Exchangeable OH not observed.

### Example 391: 1-Cyclobutyl-N-((2-(5-cyclopropyl-4′-fluoro-2′-(4-methyl-4H-1,2,4-triazol-3-yl)-[1,1′-biphenyl]-3-yl)-7-(trifluoromethyl)benzo[d]oxazol-5-yl)methyl)methanamine (JU-1)

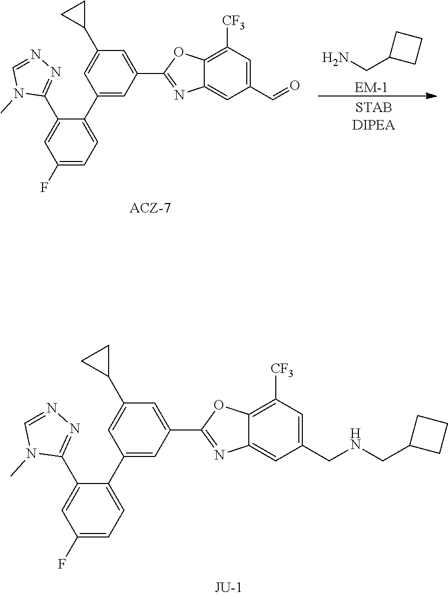

A solution of intermediate (ACZ-7) (20.0 mg, 98% Wt, 1 Eq, 38.7 μmol), (cyclobutylmethyl)amine (EM-1) (6.59 mg, 2 Eq, 77.4 μmol) and DIPEA (15.0 mg, 20.2 μL, 3 Eq, 116 μmol) in CHCl₃ (2 mL) was stirred for 12 hour at 40° C. NaBH(OAc)₃ (32.8 mg, 4 Eq, 155 μmol) was added and stirring was continued at 40° C. for another 2 hour. The reaction mixture was diluted with NaHCO₃ (5 mL) and transferred into a separating funnel. The layer was extracted with DCM (3×5 mL). The combined extracted layers were collected, dried (Na₂SO₄), filtered and concentrated in vacuo. The crude product was purified by chromatography on silica gel (4 g cartridge, 0-10% MeOH/DCM) to afford the title compound (JU-1) (12.0 mg, 21 μmol, 53%, 99% Purity) as a clear white powder. m/z 576.2 (M+H)⁺ (ES+). ¹H NMR (400 MHz, MeOD) δ 8.43 (s, 1H), 8.05-7.99 (m, 2H), 7.94 (t, J=1.7 Hz, 1H), 7.83-7.75 (m, 2H), 7.57 (td, J=8.5, 2.8 Hz, 1H), 7.48 (dd, J=8.8, 2.8 Hz, 1H), 7.00 (t, J=1.8 Hz, 1H), 3.98 (s, 2H), 3.21 (s, 3H), 2.70 (d, J=7.3 Hz, 2H), 2.58 (dt, J=15.3, 7.7 Hz, 1H), 2.18-2.08 (m, 2H), 2.06-1.83 (m, 3H), 1.75 (q, J=9.3 Hz, 2H), 1.13-1.01 (m, 2H), 0.66 (dt, J=6.7, 4.7 Hz, 2H). Exchangeable NH not observed.

### Example 392: (1S,2R)-2-[({2-[5-Cyclopropyl-4′-fluoro-2′-(4-methyl-1,2,4-triazol-3-yl)-[1,1′-biphenyl]-3-yl]-7-(trifluoromethyl)-1,3-benzoxazol-5-yl}methyl)amino]cyclopentan-1-ol (AEM-1)

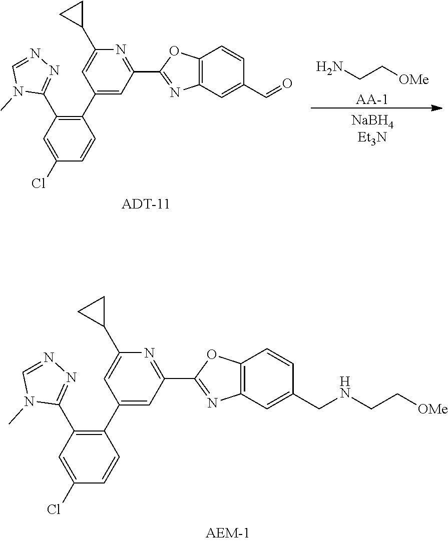

To a stirred solution of intermediate (ADT-11) (50 mg, 1 Eq, 0.11 mmol) and 2-methoxyethan-1-amine (AA-1) (8.2 mg, 1 Eq, 0.11 mmol) in MeOH (5 mL) were added Et₃N (55 mg, 5 Eq, 0.55 mmol) at rt. The resulting mixture was stirred for 2 h at rt. To the above mixture was added NaBH₄ (41 mg, 10 Eq, 1.10 mmol) at rt. The resulting mixture was stirred for 2 h at rt. The reaction was then quenched by the addition of 2 mL of MeOH at 0° C. The resulting mixture was diluted with water and extracted with DCM (3×20 mL). The combined organic layers were washed with brine (2×20 mL), dried (Na₂SO₄). After filtration, the filtrate was concentrated in vacuo. The crude product was purified by Prep-HPLC with the following conditions: Column: XBridge Prep OBD C18 Column, 30×150 mm, 5 μm; Mobile Phase A: water (0.1% NH₄HCO₃+0.1% NH₃·H₂O) Mobile Phase B: MeCN; Flow rate: 60 mL/min; Gradient: 33 B to 44 B in 9 min; Detector, UV 254/210 nm; RT: 8.19. The product-containing fractions were combined and evaporated partially in vacuo and lyophilized overnight to afford the title compound (AEM-1) (9.1 mg, 18 μmol, 16%, 99.8% Purity) as a white solid. m/z 515.2/517.2 (M+H)⁺ (ES+). ¹H NMR (400 MHz, Methanol-d4) δ 8.45 (s, 1H), 7.90 (d, J=1.6 Hz, 1H), 7.85-7.74 (m, 4H), 7.69 (d, J=8.4 Hz, 1H), 7.48 (m, 1H), 7.10 (d, J=1.6 Hz, 1H), 3.94 (s, 2H), 3.53 (t, J=5.4 Hz, 2H), 3.35 (s, 3H), 3.29 (s, 3H), 2.80 (t, J=5.4 Hz, 2H), 2.18-2.13 (m, 1H), 1.07 (s, 4H).

### Example 393: 4-[2-Cyclopropyl-6-(5-{1[(2-methoxyethyl)amino]methyl}-1,3-benzoxazol-2-yl)pyridin-4-yl]-3-(4-methyl-1,2,4-triazol-3-yl)benzonitrile (AEN-15)

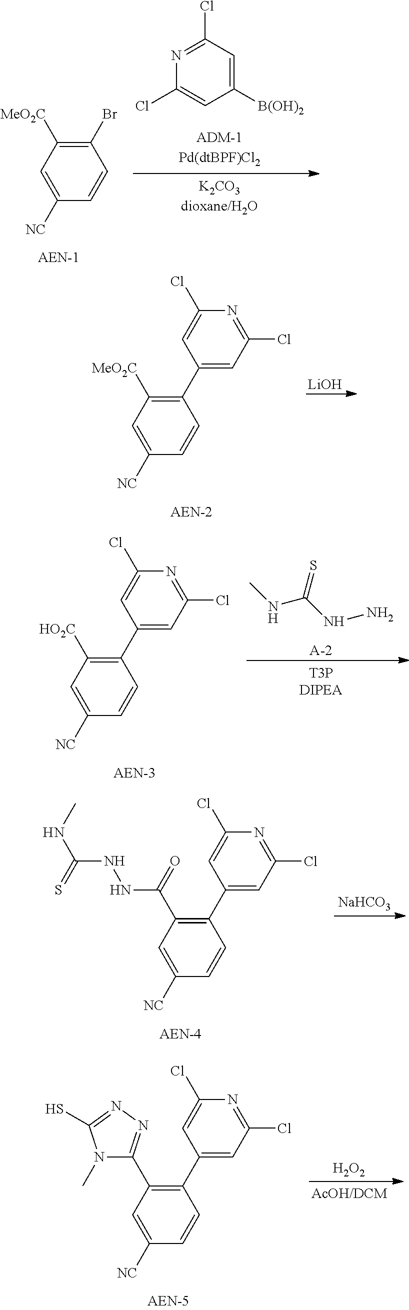

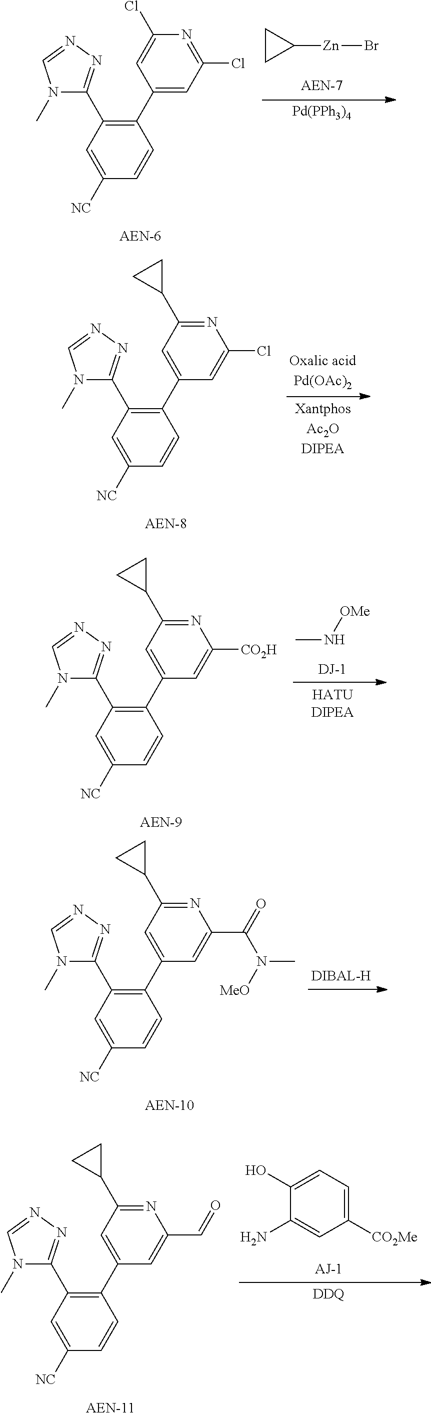

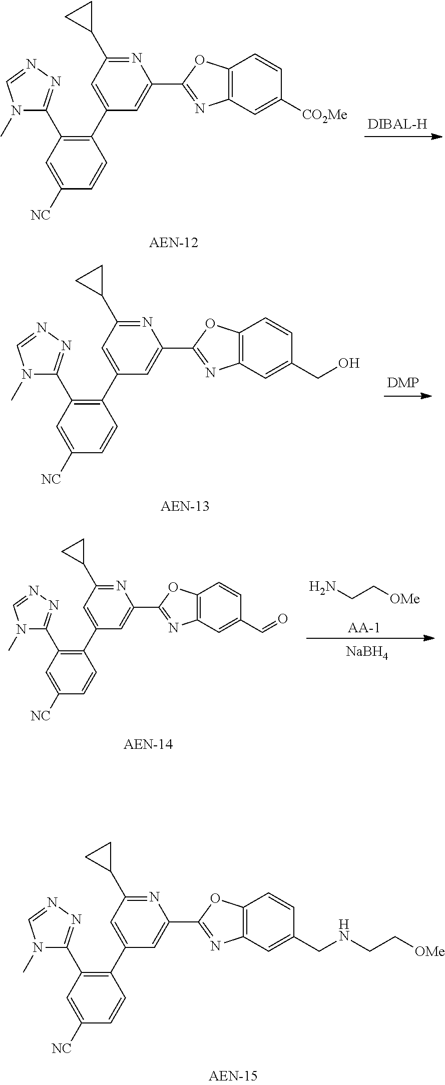

### Step 1: Methyl 5-cyano-2-(2,6-dichloropyridin-4-yl) benzoate (AEN-2)

To a stirred solution of methyl 2-bromo-5-cyanobenzoate (AEN-1) (2.40 g, 1 eq, 10.0 mmol), 2,6-dichloropyridin-4-ylboronic acid (ADM-1) (1.92 g, 1 eq, 10.0 mmol) and K₂CO₃ (4.15 g, 3 eq, 30 mmol) in 1,4-dioxane (30 mL) and H₂O (3 mL) was added Pd(dtBPF)Cl₂ (652 mg, 0.1 eq, 1.00 mmol) at rt under nitrogen atmosphere. The resulting mixture was stirred for 3 h at 60° C. under nitrogen atmosphere. The mixture was allowed to cool down to rt. The resulting mixture was diluted with water and extracted with EtOAc (3×200 mL). The combined organic layers were washed with brine (2×200 mL), dried (Na₂SO₄). After filtration, the filtrate was concentrated in vacuo. The residue was applied on a silica gel column chromatography with petroleum ether/EtOAc (4/1) to afford the sub-title compound (AEN-2) (2.2 g, 5.96 mmol, 72%, 83% Purity) as an off-white solid. m/z 307.0/309.0 (M+H)⁺ (ES+).

Step 2: 5-Cyano-2-(2,6-dichloropyridin-4-yl) benzoic acid (AEN-3). To a stirred solution of the product from step 1 above (AEN-2) (2.20 g, 1 eq, 7.16 mmol) and LiOH (860 mg, 5 eq, 35.8 mmol) in THF (30 mL) and H₂O (10 mL) at rt. The resulting mixture was stirred for 3 h at rt. The mixture was acidified to pH3 with aq. of HCl (4M) at 0° C. The resulting mixture was diluted with water and extracted with EtOAc (3×200 mL). The combined organic layers were washed with brine (2×200 mL), dried (Na₂SO₄). After filtration, the filtrate was concentrated in vacuo to afford the sub-title compound (AEN-3) (2.1 g, 6.11 mmol, 95%, 85% Purity) as an off-white solid. m/z 293.0/295.0 (M+H)⁺ (ES+)

Step 3: 5-Cyano-2-(2,6-dichloropyridin-4-yl)-N-[(methylcarbamothioyl)amino]benzamide (ADP-4). To a stirred solution of the product from step 2 above (AEN-3) (2.10 g, 1 eq, 7.17 mmol), 4-methyl-3-thiosemicarbazide (A-2) (753 mg, 1 eq, 7.17 mmol), T₃P (50% in DMF) (9.12 g, 50% Wt, 4 eq, 28.7 mmol) and DIPEA (5.56 g, 6 eq, 43.0 mmol) in DMF (20 mL) at rt. The resulting mixture was stirred for 3 h at rt. The crude product was purified by reverse flash column chromatography with the following conditions: Column, C18; mobile phase, water (0.1% NH₄HCO₃) and MeCN (0% MeCN up to 40% in 24 min); Detector, UV 254/220 nm. This resulted in the sub-title compound (AEN-4) (1.9 g, 4.51 mmol, 70%, 90% Purity) as an off-white solid. m/z 380.0/382.0 (M+H)⁺ (ES+).

Step 4: 4-(2,6-Dichloropyridin-4-yl)-3-(4-methyl-5-sulfanyl-1,2,4-triazol-3-yl)benzonitrile (AEN-5). A solution of the product from step 3 above (AEN-4) (1.90 g, 1 eq, 5.00 mmol) in aq. of NaHCO₃ (20 mL, 1 M) was stirred for 1 h at 80° C. The mixture was allowed to cool down to rt. The crude product was purified by reverse flash column chromatography with the following conditions: Column, C18; mobile phase, water (0.1% NH₄HCO₃) and MeCN (0% MeCN up to 40% in 25 min); Detector, UV 254/220 nm. This resulted in the sub-title compound (AEN-5) (1.3 g, 3.42 mmol, 61%, 95% Purity) as an off-white solid. m/z 362.0/364.0 (M+H)⁺ (ES+)

Step 5: 4-(2, 6-Dichloropyridin-4-yl)-3-(4-methyl-1,2,4-triazol-3-yl)benzonitrile (AEN-6). To a stirred solution of the product from step 4 above (AEN-5) (1.30 g, 1 eq, 3.59 mmol), H₂02 (610 mg, 30% Wt, 5 eq, 17.9 mmol), AcOH (431 mg, 2 eq, 7.18 mmol) in DCM (20 mL) at rt. The resulting mixture was stirred for 2 h at rt. The crude product was purified by reverse flash column Chromatography with the following conditions: Column, C18; mobile phase, water (0.1% NH₄HCO₃) and MeCN (0% MeCN up to 45% in 25 min); Detector, UV 254/220 nm. This resulted in the sub-title compound (AEN-6) (750 mg, 2.05 mmol, 63%, 90% Purity) as an off-white solid. m/z 330.0/332.0 (M+H)⁺ (ES+)

Step 6: 4-(2-Chloro-6-cyclopropylpyridin-4-yl)-3-(4-methyl-1,2,4-triazol-3-yl)benzonitrile (AEN-8). To a stirred mixture of the product from step 5 above (AEN-6) (80 mg, 1 eq, 0.24 mmol) and bromo(cyclopropyl)zinc (AEN-7) (226 mg, 5 eq, 1.21 mmol) in THF (3 mL) were added Pd(PPh₃)₄ (56 mg, 0.2 eq, 48 μmol) at rt under nitrogen atmosphere. The resulting mixture was stirred for 2 h at 100° C. under nitrogen atmosphere. The mixture was allowed to cool down to rt. The reaction was then quenched by the addition of 2 mL of ice water at 0° C. The crude product was purified by reverse flash column chromatography with the following conditions: Column, C18; mobile phase, water (0.1% NH₄HCO₃) and MeCN (10% MeCN up to 60% in 25 min); Detector, UV 254/220 nm to afford the sub-title compound (AEN-8) (25 mg, 69 μmol, 31%, 93% Purity) as a white solid. m/z 336.1/338.1 (M+H)⁺ (ES+)

Step 7: 4-[4-Cyano-2-(4-methyl-1,2,4-triazol-3-yl)phenyl]-6-cyclopropylpyridine-2-carbox ylic acid (AEN-9). To a stirred solution of the product from step 6 above (AEN-8) (600 mg, 1 Eq, 1.79 mmol) and oxalic acid (242 mg, 1.5 Eq, 2.68 mmol) in DMF (7 mL) were added Ac₂0 (274 mg, 2.680 mmol, 1.5 Eq) and DIPEA (346 mg, 1.5 Eq, 2.68 mmol) at rt under nitrogen atmosphere. To the above mixture was added Xantphos (207 mg, 0.2 Eq, 0.36 mmol) and Pd(OAc)₂ (40 mg, 0.1 Eq, 0.18 mmol) at rt under nitrogen atmosphere. The resulting mixture was stirred for 12 h at 100° C. under nitrogen atmosphere. The mixture was allowed to cool down to rt. The crude product was purified by reverse flash column chromatography with the following conditions: Column, C18; mobile phase, water (0.1% NH₄HCO₃) and MeCN (10% MeCN up to 15% in 15 min); Detector, UV 254/220 nm. This resulted in the sub-title compound (AEN-9) (350 mg, 1.01 mmol, 57%, 90% Purity) as a white solid. m/z 346.1 (M+H)⁺ (ES+)

Step 8: 4-[4-cyano-2-(4-methyl-1,2,4-triazol-3-yl)phenyl]-6-cyclopropyl-N-methoxy-N-methylpyridine-2-carboxamide (AEN-10). To a stirred solution of the product from step 7 above (AEN-9) (350 mg, 1 Eq, 1.01 mmol) and N,O-dimethylhydroxylamine (DJ-1) (74 mg, 1.2 Eq, 1.22 mmol) in DMF (10 mL) were added DIPEA (393 mg, 3 Eq, 3.04 mmol) and HATU (501 mg, 1.3 Eq, 1.32 mmol) at rt. The resulting mixture was stirred for 2 h at rt. The resulting mixture was diluted with water and extracted with EtOAc (3×50 mL). The combined organic layers were washed with brine (2×50 mL), dried (Na₂SO₄). After filtration, the filtrate was concentrated in vacuo. The residue was purified by TLC with DCM/MeOH (12/1) to afford the sub-title compound (AEN-10) (330 mg, 0.85 mmol, 84%, 92% Purity) as a colourless oil. m/z 389.2 (M+H)⁺ (ES+)

Step 9: 4-(2-Cyclopropyl-6-formylpyridin-4-yl)-3-(4-methyl-1,2,4-triazol-3-yl)benzonitrile (AEN-11). To a stirred solution of the product from step 8 above (AEN-10) (250 mg, 1 Eq, 0.64 mmol) in THF (10 mL) was added DIBAL-H (g) in THF (5.8 mL, 1 M, 9 Eq, 5.76 mmol) in portions at 0° C. under nitrogen atmosphere. The resulting mixture was stirred for 1 h at rt under nitrogen atmosphere. The reaction was then quenched by the addition of 2 mL of ice water at 0° C. The reaction was concentrated in vacuo. The residue was purified by TLC with DCM/MeOH (12/1) to afford the sub-title compound (AEN-11) (120 mg, 0.36 mmol, 57%, 92% Purity) as a white solid. m/z 330.1 (M+H)⁺ (ES+).

Step 10: Methyl 2-{4-[4-cyano-2-(4-methyl-1,2,4-triazol-3-yl)phenyl]-6-cyclopropylpyridin-2-yl}-1,3-benzoxazole-5-carboxylate (AEN-12). A solution of the product from step 9 above (AEN-11) (120 mg, 1 Eq, 0.36 mmol) and methyl 3-amino-4-hydroxybenzoate (AJ-1) (61 mg, 1 Eq, 0.36 mmol) in DCM (10 mL) was stirred for 1 h at 60° C. The mixture was allowed to cool down to rt. To the above mixture was added DDQ (165 mg, 2 Eq, 0.73 mmol) at rt. The resulting mixture was stirred for additional 1 h at rt. The resulting mixture was diluted with water and extracted with EtOAc (3×20 mL). The combined organic layers were washed with brine (2×20 mL), dried (Na₂SO₄). After filtration, the filtrate was concentrated in vacuo. The residue was purified by TLC with DCM/MeOH (10/1) to afford the sub-title compound (AEN-12) (100 mg, 0.21 mmol, 58%, 93% Purity) as a brown solid. m/z 477.2 (M+H)⁺ (ES+)

Step 11: 4-{2-Cyclopropyl-6-[5-(hydroxymethyl)-1,3-benzoxazol-2-yl]pyridin-4-yl}-3-(4-methyl-1,2,4-triazol-3-yl)benzonitrile (AEN-13). To a stirred solution of the product from step 10 above (AEN-12) (80 mg, 1 Eq, 0.17 mmol) in THF (5 mL) was added DIBAL-H (g) in THF (0.5 mL, 1 M, 3 Eq, 0.51 mmol) dropwise at 0° C. under nitrogen atmosphere. The resulting mixture was stirred for 1 h at 0° C. under nitrogen atmosphere. The reaction was then quenched by the addition of 3 mL of ice water at 0° C. and concentrated in vacuo. The residue was purified by TLC with DCM/MeOH (10/1) to afford the sub-title compound (AEN-13) (35 mg, 78 μmol, 46%, 95% Purity) as a brown solid. m/z 449.2 (M+H)⁺ (ES+)

Step 12: 4-[2-Cyclopropyl-6-(5-formyl-1,3-benzoxazol-2-yl)pyridin-4-yl]-3-(4-methyl-1,2,4-triazol-3-yl)benzonitrile (AEN-14). To a stirred solution of the product from step 11 above (AEN-13) (35 mg, 1 Eq, 0.08 mmol) in DCM (5 mL) was added DMP (44 mg, 1.3 Eq, 0.10 mmol) at rt. The resulting mixture was stirred for 1 h at rt. The resulting mixture was filtered; the filter cake was washed with DCM (3×2 mL). The filtrate was concentrated in vacuo. The crude product was used in the next step directly without further purification. m/z 447.2 (M+H)⁺ (ES+)

Step 13: 4-[2-Cyclopropyl-6-(5-{[(2-methoxyethyl)amino]methyl}-1,3-benzoxazol-2-yl)pyridin-4-yl]-3-(4-methyl-1,2,4-triazol-3-yl) benzonitrile (AEN-15). A solution of the product from step 12 above (AEN-14) (20 mg, 1 Eq, 45 μmol) and 2-methoxyethan-1-amine (AA-1) (5.1 mg, 1.5 Eq, 68 μmol) in MeOH (5 mL) was stirred for 1 h at 60° C. The mixture was cooled to rt. To the above mixture was added NaBH₄ (4 mg, 2 Eq, 0.09 mmol) at 0° C. The resulting mixture was stirred for additional 1 h at rt. The reaction was then quenched by the addition of 2 mL of MeOH at 0° C. and then concentrated in vacuo. The residue was purified by TLC with DCM/MeOH (10/1). The crude product was purified by Prep-HPLC with the following conditions (Column: XBridge Prep OBD C18 Column, 30*150 mm, 5 μm; Mobile Phase A: water (0.1% NH₄HCO₃+0.1% NH₃·H₂O), Mobile Phase B: MeCN; Flow rate: 60 mL/min; Gradient: 30% B to 40% B in 8 min; Wave Length: 254/220 nm) to afford the title compound (AEN-15) (6.5 mg, 13 μmol, 29%, 99.5% Purity) as a white solid. m/z 506.1 (M+H)⁺ (ES+). ¹H NMR (400 MHz, Methanol-d4) δ 8.48 (d, J=2.0 Hz, 1H), 8.19-8.09 (m, 2H), 8.01-7.95 (m, 1H), 7.92 (d, J=1.7 Hz, 1H), 7.78 (d, J=1.7 Hz, 1H), 7.70-7.65 (m, 1H), 7.51-7.45 (m, 1H), 7.15 (d, J=1.8 Hz, 1H), 3.92 (d, J=2.0 Hz, 2H), 3.52 (t, J=5.4 Hz, 2H), 3.35-3.33 (m, 6H), 2.78 (t, J=5.3 Hz, 2H), 2.23-2.14 (m, 1H), 1.12-1.00 (m, 4H).

### Example 394: 4-[2-(5-{[(Cyclobutylmethyl)amino]methyl}-1,3-benzoxazol-2-yl)-6-cyclopropylpyridin-4-yl]-3-(4-methyl-1,2,4-triazol-3-yl)benzonitrile (AEO-1)

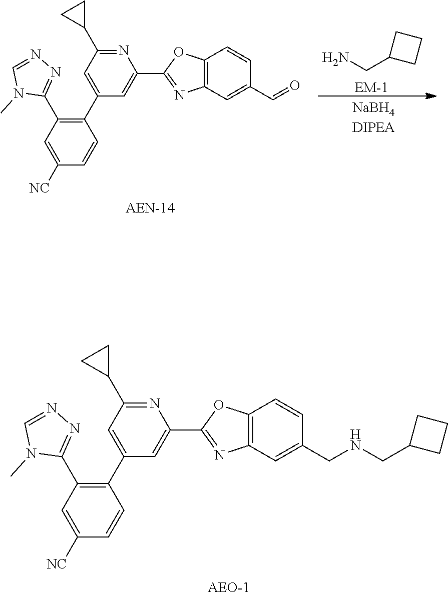

To a stirred solution of intermediate (AEN-14) (15 mg, 1 Eq, 34 μmol) and 1-cyclobutylmethanamine (EM-1) (59 mg, 1.5 Eq, 51 μmol) in MeOH (5 mL) was added DIPEA (5.2 mg, 1.2 Eq, 41 μmol) dropwise at rt. The resulting mixture was stirred for 1 h at 60° C. The mixture was cooled to rt. To the above mixture was added NaBH₄ (3 mg, 2 Eq, 68 μmol) at 0° C. The resulting mixture was stirred for additional 1 h at rt. The reaction was then quenched by the addition of 2 mL of MeOH at 0° C. and then concentrated in vacuo. The residue was purified by TLC with DCM/MeOH (10/1). The crude product was purified by Prep-HPLC with the following conditions (Column: XBridge Prep OBD C18 Column, 30*150 mm, 5 μm; Mobile Phase A: water (0.1% NH₄HCO₃+0.1% NH₃·H₂O), Mobile Phase B: MeCN; Flow rate: 60 mL/min; Gradient: 39% B to 49% B in 8 min; Wave Length: 254/220 nm) to afford the title compound (AEO-1) (0.5 mg, 1.0 μmol, 2.9%, 99.5% Purity) as a white solid. m/z 516.2 (M+H)⁺ (ES+). ¹H NMR (400 MHz, Methanol-d4) δ 8.38 (d, J=2.2 Hz, 1H), 8.09-8.04 (m, 1H), 8.02 (s, 1H), 7.87 (d, J=8.1 Hz, 1H), 7.82 (s, 1H), 7.68 (s, 1H), 7.58 (d, J=8.3 Hz, 1H), 7.38 (d, J=8.3 Hz, 1H), 7.06 (s, 1H), 3.78 (s, 2H), 3.23 (d, J=1.9 Hz, 3H), 2.53 (d, J=7.2 Hz, 2H), 2.47-2.40 (m, 1H), 2.10-1.98 (m, 3H), 1.85-1.75 (m, 2H), 1.70-1.50 (m, 2H), 1.01-0.93 (m, 4H).

### Example 395: {2-[5-cyclopropyl-4′-fluoro-2′-(4-methyl-1,2,4-triazol-3-yl)-[1,1′-biphenyl]-3-yl]-1,3-benzoxazol-5-yl}methanol (AEP-2)

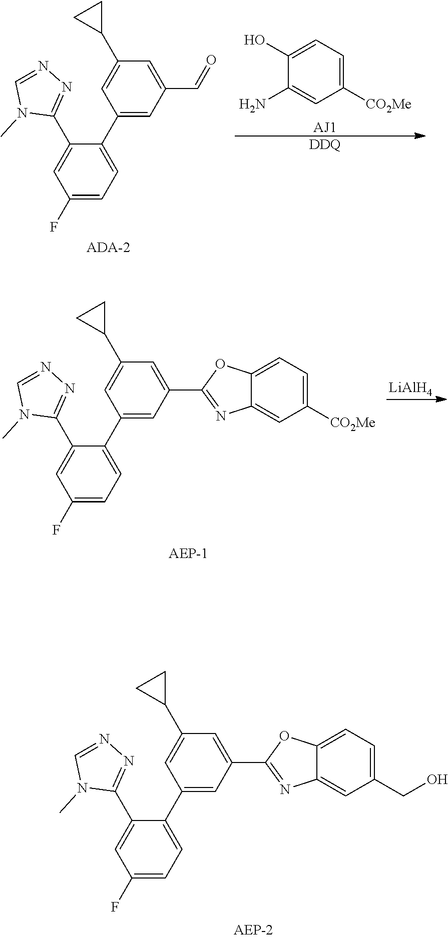

Step 1: Methyl 2-[5-cyclopropyl-4′-fluoro-2′-(4-methyl-1,2,4-triazol-3-yl)-[1,1′-biphenyl]-3-yl]-1,3-benzoxazole-5-carboxylate (AEP-1). To a stirred solution of intermediate (ACZ-4) (560 mg, 1 Eq, 1.74 mmol) and methyl 3-amino-4-hydroxybenzoate (AJ-3) (350 mg, 1.2 Eq, 2.09 mmol) in DCM (10 mL) at rt. The resulting mixture was stirred for 1 h at 60° C. The mixture was cooled to rt. To the above mixture was added DDQ (791 mg, 2 Eq, 3.48 mmol) at rt. The resulting mixture was stirred for 2 h at rt. The resulting mixture was diluted with water and extracted with EtOAc (3×50 mL). The combined organic layers were washed with brine (2×50 mL), dried (Na₂SO₄). After filtration, the filtrate was concentrated in vacuo. The crude product was purified by reverse flash column chromatography with the following conditions: Column, C18; mobile phase, water (0.1% FA) and MeCN (20% MeCN up to 50% in 10 min); Detector, UV 254/220 nm. The product-containing fractions were combined and concentrated in vacuo. This resulted in the sub-title compound (AEP-1) (320 mg, 0.68 mmol, 39%, 90% Purity) as a yellow solid. m/z 469.2 (M+H)⁺ (ES+).

Step 2: {2-[5-Cyclopropyl-4′-fluoro-2′-(4-methyl-1,2,4-triazol-3-yl)-[1,1′-biphenyl]-3-yl]-1,3-benzoxazol-5-yl}methanol (AEP-2). To a stirred solution of the product from step 1 above (AEP-1) (30 mg, 1 Eq, 64 μmol) in THF (5 mL) was added DIBAl-H (g) in THF (0.5 mL, 1 m, 8 Eq, 0.51 mmol) at 0° C. under nitrogen atmosphere. The resulting mixture was stirred for 0.5 h at 0° C. under nitrogen atmosphere. The reaction was then quenched by the addition of 3 mL of ice water at 0° C. The resulting mixture was diluted with water and extracted with EtOAc (3×10 mL). The combined organic layers were washed with brine (2×10 mL), dried (Na₂SO₄). After filtration, the filtrate was concentrated in vacuo. The residue was purified by Prep-TLC with EtOAc/petroleum ether (1/1). The crude product was purified by Prep-HPLC with the following conditions: Column: XBridge Prep OBD C18 Column, 30×150 mm, 5 μm; Mobile Phase A: water (0.1% NH₄HCO₃+0.1% NH₃·H₂O) Mobile Phase B: MeCN; Flow rate: 60 mL/min; Gradient: 36 B to 46 B in 8 min; Detector, UV 254/210 nm; RT: 7.23. The product-containing fractions were combined and evaporated partially in vacuo and lyophilized overnight to afford the title compound (AEP-2) (4.4 mg, 10.0 μmol, 16%, 99.6% Purity) as a white solid. m/z 441.1 (M+H)⁺ (ES+). ¹H NMR (400 MHz, Methanol-d4) δ 8.40 (s, 1H), 7.95 (t, J=1.7 Hz, 1H), 7.92 (t, J=1.7 Hz, 1H), 7.80-7.71 (m, 2H), 7.64 (d, J=8.3 Hz, 1H), 7.57-7.49 (m, 1H), 7.47-7.40 (m, 2H), 6.93 (t, J=1.7 Hz, 1H), 4.73 (s, 2H), 3.13 (s, 3H), 2.04-1.88 (m, 1H), 1.07-0.96 (m, 2H), 0.69-0.54 (m, 2H).

### Example 396: (1S,2R)-2-[({2-[5-Cyclopropyl-4′-fluoro-2′-(4-methyl-1,2,4-triazol-3-yl)-[1,1′-biphenyl]-3-yl]-1,3-benzoxazol-5-yl}methyl)amino]cyclopentan-1-ol (AEQ-2)

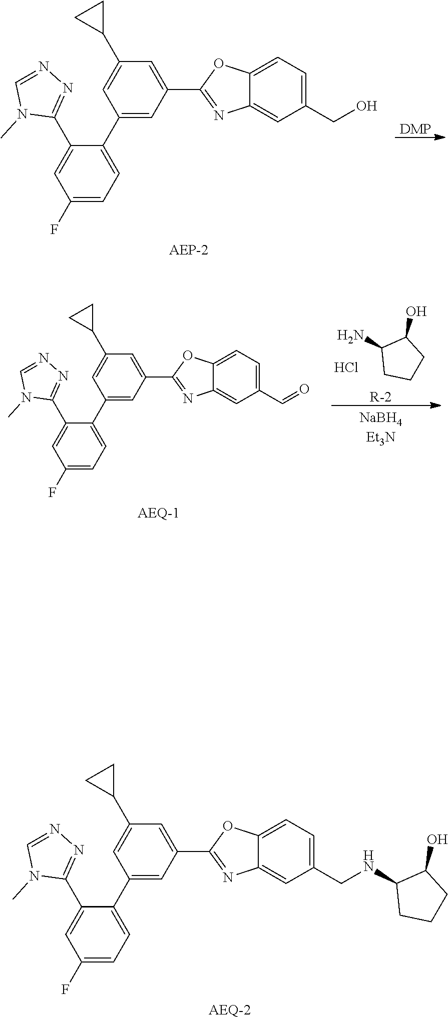

Step 1: 2-[5-Cyclopropyl-4′-fluoro-2′-(4-methyl-1,2,4-triazol-3-yl)-[1,1′-biphenyl]-3-yl]-1,3-benzoxazole-5-carbaldehyde (AEQ-1). To a solution of intermediate (AEP-2) (100 mg, 1 Eq, 0.22 mmol) in DCM (5 mL) was added DMP (125 mg, 1.3 Eq, 0.30 mmol) at rt. The resulting mixture was stirred for 2 h at rt. The resulting mixture was filtered the filter cake was washed with EtOAc (3×5 mL). The filtrate was concentrated in vacuo. This resulted in the sub-title compound (AEQ-1) (90 mg, 0.21 mmol, 90%, 85% Purity) as a white solid. m/z 439.2 (M+H)⁺ (ES+).

Step 2: (1S,2R)-2-[({2-[5-Cyclopropyl-4′-fluoro-2′-(4-methyl-1,2,4-triazol-3-yl)-[1,1′-biphenyl]-3-yl]-1,3-benzoxazol-5-yl}methyl)amino]cyclopentan-1-ol (AEQ-2). To a stirred solution of the product from step 1 above (AEQ-1) (50 mg, 1 Eq, 0.11 mmol) and (1S,2R)-2-aminocyclopentan-1-ol, HCl (R-2) (11 mg, 1.1 Eq, 0.10 mmol) in DCM (5 mL) was added Et₃N (58 mg, 5 Eq, 0.57 mmol) for 2 h at 60° C. under nitrogen atmosphere. The mixture was cooled to rt. To the above mixture was added NaBH₄ (43 mg, 10 Eq, 1.14 mmol) at 0° C. The resulting mixture was stirred for 2 hours at rt. The reaction was then quenched by the addition of 2 mL of MeOH at 0° C. The resulting mixture was diluted with water and extracted with DCM (3×10 mL). The combined organic layers were washed with brine (2×10 mL), dried (Na₂SO₄). After filtration, the filtrate was concentrated in vacuo. The crude product was purified by Prep-HPLC with the following conditions: Column: XBridge Prep OBD C18 Column, 19×250 mm, 5 μm; Mobile Phase A: water (0.1% NH₄HCO₃) Mobile Phase B: MeCN; Flow rate: 25 mL/min; Gradient: 42 B to 52 B in 8 min; Detector, UV 254/210 nm; RT: 5.7. The product-containing fractions were combined and evaporated partially in vacuo and lyophilized overnight to afford the title compound (AEQ-2) (4.1 mg, 7.8 μmol, 6.8%, 99.5% Purity) as a white solid. m/z 524.1 (M+H)⁺ (ES+). ¹H NMR (400 MHz, Methanol-d4) δ 8.40 (s, 1H), 7.94-7.91 (m, 2H), 7.81-7.72 (m, 2H), 7.63 (d, J=8.4 Hz, 1H), 7.61-7.52 (m, 1H), 7.46-7.42 (m, 2H), 6.92 (t, J=1.7 Hz, 1H), 4.16-4.15 (m, 1H), 3.98-3.90 (m, 2H), 3.12 (s, 3H), 3.05-2.90 (m, 1H), 2.03-1.88 (m, 2H), 1.86-1.66 (m, 3H), 1.61-1.48 (m, 2H), 1.08-0.98 (m, 2H), 0.64-0.62 (m, 2H).

### Example 397: (1S,2R)-2-(((2-(4-(4-Chloro-2-(4-methyl-4H-1,2,4-triazol-3-yl)phenyl)pyridin-2-yl)-7-(trifluoromethyl)benzo[d]oxazol-5-yl)methyl)amino)cyclopentan-1-ol (AER-8)

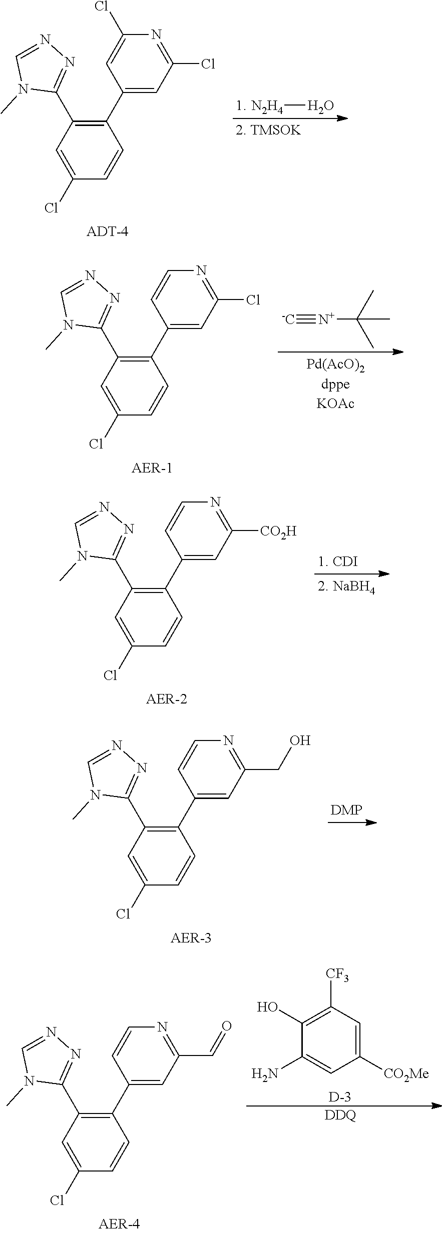

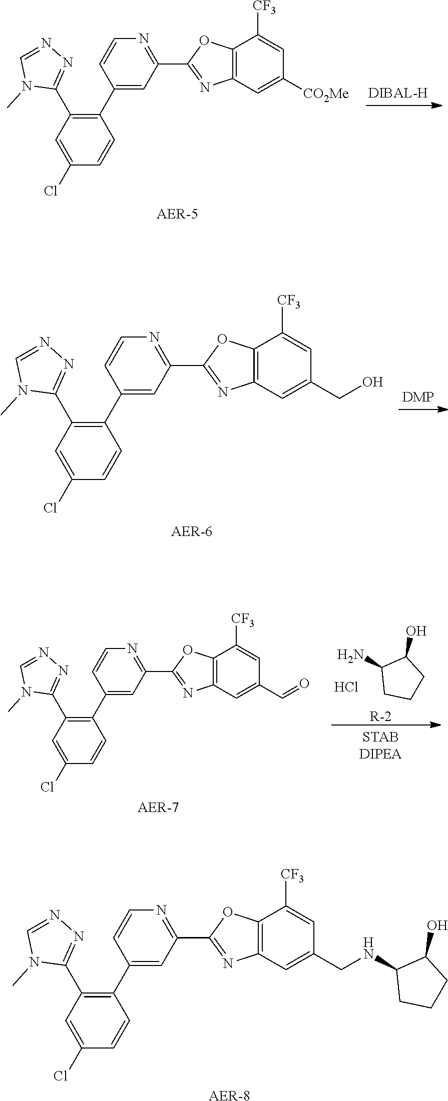

Step 1: 2-Chloro-4-(4-chloro-2-(4-methyl-4H-1,2,4-triazol-3-yl)phenyl)pyridine (AER-1). To a stirred solution of intermediate (ADT-4) (3.00 g, 1 Eq, 8.83 mmol) in EtOH (30 mL) was added aq. of hydrazine hydrate (13.3 g, 98% Wt, 30 Eq, 265 mmol) at rt. The resulting mixture was stirred for 5 h at 80° C. The mixture was allowed to cool down to rt and concentrated in vacuo. The crude intermediate is dissolved in DMF (30 mL). To the above mixture was added TMSOK (4.53 g, 4 Eq, 35.3 mmol) at 0° C. The resulting mixture was stirred for additional overnight at rt. The resulting mixture was diluted with water and extracted with EtOAc (3×200 mL). The combined organic layers were washed with brine (2×200 mL), dried (Na₂SO₄). After filtration, the filtrate was concentrated in vacuo. The crude product was purified by reverse flash column chromatography with the following conditions: Column, C18; mobile phase, water (0.1% NH₄HCO₃) and ACN (10% MeCN up to 80% in 20 min); Detector, UV 254/220 nm. The product-containing fractions were combined and evaporated to afford the sub-title compound (AER-1) (2.0 g, 6.58 mmol, 74%, 90% Purity) as a brown solid. m/z 305.0/307.0 (M+H)⁺ (ES+).

Step 2: 4-(4-Chloro-2-(4-methyl-4H-1,2,4-triazol-3-yl)phenyl)picolinic acid (AER-2). To a stirred mixture of the product from step 1 above (AER-1) (300 mg, 1 Eq, 0.98 mmol) and tert-butyl isocyanide (98 mg, 1.2 Eq, 1.18 mmol) in DMSO (5 mL) were added KoAC (193 mg, 2 Eq, 1.97 mmol), DPPE (78 mg, 0.2 Eq, 0.20 mmol) and Pd(AcO)₂ (22 mg, 0.1 Eq, 0.10 mmol) at rt under nitrogen atmosphere. The resulting mixture was stirred for overnight at 120° C. under nitrogen atmosphere. The mixture was allowed to cool down to rt. The crude product was purified by reverse flash column chromatography with the following conditions: Column, C18; mobile phase, water (0.1% FA) and MeCN (10% MeCN up to 80% in 20 min); Detector, UV 254/220 nm. The product-containing fractions were combined and evaporated to afford the sub-title compound (AER-2) (200 mg, 0.63 mmol, 65%, 92% Purity) as a white solid. m/z 315.1/317.1 (M+H)⁺ (ES+).

Step 3: (4-(4-Chloro-2-(4-methyl-4H-1,2,4-triazol-3-yl)phenyl)pyridin-2-yl)methanol (AER-3). To a stirred solution of the product from step 2 above (AER-2) (200 mg, 1 Eq, 0.64 mmol) in THF (10 mL) was added CDI (515 mg, 5 Eq, 3.18 mmol) at rt under nitrogen atmosphere. The resulting mixture was stirred for 4 h at 60° C. under nitrogen atmosphere. The mixture was allowed to cool down to rt. To the above mixture was added a mixture of NaBH₄ (72 mg, 3 Eq, 1.91 mmol) in THF (2 mL) at 0° C. The resulting mixture was stirred for additional 2 h at rt. The resulting mixture was concentrated in vacuo. The residue was purified by Prep-TLC with DCM/MeOH (30/1) to afford the sub-title compound (AER-3) (140 mg, 0.47 mmol, 73%, 91% Purity) as a brown solid. m/z 301.1/303.1 (M+H)⁺ (ES+).

Step 4: 4-(4-Chloro-2-(4-methyl-4H-1,2,4-triazol-3-yl)phenyl)picolinaldehyde (AER-4). To a stirred solution of the product from step 4 above (AER-3) (120 mg, 1 Eq, 0.40 mmol) in DCM (10 mL) was added DMP (203 mg, 1.2 Eq, 0.48 mmol) at 0° C. The resulting mixture was stirred for 2 h at rt. The resulting mixture was concentrated in vacuo. The residue was purified by Prep-TLC with DCM/MeOH (30/1) to afford the sub-title compound (AER-4) (100 mg, 0.34 mmol, 84%, 90% Purity) as a brown solid. m/z 299.1/301.1 (M+H)⁺ (ES+).

Step 5: Methyl 2-(4-(4-chloro-2-(4-methyl-4H-1,2,4-triazol-3-yl)phenyl)pyridin-2-yl)-7-(trifluoromethyl)benzo[d]oxazole-5-carboxylate (AER-5). A mixture of the product from step 4 above (AER-4) (100 mg, 1 Eq, 0.34 mmol) and intermediate (D-3) (79 mg, 1 Eq, 0.34 mmol) in DCM (22 mL) was stirred for overnight at 60° C. under nitrogen atmosphere. The mixture was allowed to cool down to rt. To the above mixture was added DDQ (91 mg, 1.2 Eq, 0.40 mmol) at rt. The resulting mixture was stirred for additional 2 h at rt under nitrogen atmosphere. The resulting mixture was concentrated in vacuo. The residue was purified by Prep-TLC with DCM/MeOH (15/1) to afford the sub-title compound (AER-5) (75 mg, 0.15 mmol, 44%, 90% Purity) as a brown solid. m/z 514.1/516.1 (M+H)⁺ (ES+).

Step 6: (2-(4-(4-Chloro-2-(4-methyl-4H-1,2,4-triazol-3-yl)phenyl)pyridin-2-yl)-7-(trifluoromethyl)benzo[d]oxazol-5-yl)methanol (AER-6). To a stirred solution of the product from step 5 above (AER-5) (70 mg, 1 Eq, 0.14 mmol) in THF (14 mL) at −40° C. under nitrogen atmosphere. To the above mixture was added DIBAL-H in THF (0.4 mL, 1 M, 3 Eq, 0.41 mmol) dropwise at −40° C. under nitrogen atmosphere. The resulting mixture was stirred for 2 h at −40° C. to −5° C. under nitrogen atmosphere. The reaction was then quenched by the addition of 2 mL of ice water at 0° C. The resulting mixture was concentrated in vacuo. The residue was purified by Prep-TLC with DCM/MeOH (10/1) to afford the sub-title compound (AER-6) (35 mg, 72 μmol, 53%, 95% Purity) as a brown solid. m/z 486.1/488.1 (M+H)⁺ (ES+).

Step 7: 2-(4-(4-Chloro-2-(4-methyl-4H-1,2,4-triazol-3-yl)phenyl)pyridin-2-yl)-7-(trifluoromethyl)benzo[d]oxazole-5-carbaldehyde (AER-7). To a stirred solution of the product from step 6 above (AER-6) (35 mg, 1 Eq, 72 μmol) in DCM (7 mL) was added DMP (37 mg, 1.2 Eq, 86 μmol) at 0° C. The resulting mixture was stirred for 2 h at rt. The resulting mixture was concentrated in vacuo. The residue was purified by Prep-TLC with DCM/MeOH (30/1) to afford the sub-title compound (AER-7) (35 mg, 72 μmol, 98%, 90% Purity) as a brown solid. m/z 484.1/486.1 (M+H)⁺ (ES+).

Step 8: (1S,2R)-2-(((2-(4-(4-Chloro-2-(4-methyl-4H-1,2,4-triazol-3-yl)phenyl)pyridin-2-yl)-7-(trifluoromethyl)benzo[d]oxazol-5-yl)methyl)amino)cyclopentan-1-ol (AER-8). To a stirred mixture of the product from step 7 above (AER-7) (35 mg, 1 Eq, 72 μmol) and (1S,2R)-2-aminocyclopentan-1-ol, HCl (R-2) (30 mg, 3 Eq, 0.22 mmol) in DCM (7 mL) was added DIPEA (28 mg, 3 Eq, 0.22 mmol) at rt. The resulting mixture was stirred for 0.5 h at rt. To the above mixture was added AcOH (13 mg, 3 Eq, 0.22 mmol) and NaBH(OAc)₃ (46 mg, 3 Eq, 0.22 mmol) at 0° C. The resulting mixture was stirred for overnight at rt. The reaction was then quenched by the addition of 2 mL of MeOH at 0° C. The residue was purified by Prep-TLC with DCM/MeOH (10/1) and then purified by Prep-HPLC with the following conditions: (Column: Xselect CSH C18 OBD Column 30*150 mm, 5 μm; Mobile Phase A: water (0.1% FA), Mobile Phase B: MeCN; Flow rate: 60 mL/min; Gradient: 2% B to 28% B in 10 min; Wave Length: 254 nm; RT: 10.47). The product-containing fractions were combined and evaporated partially in vacuo and lyophilized overnight to afford the title compound (AER-8) (3.7 mg, 6.5 μmol, 8.8%, 97.8% Purity) as a white solid. m/z 569.1/571.1 (M+H)⁺ (ES+). ¹H NMR (400 MHz, Methanol-d4) δ 8.74 (d, J=5.1 Hz, 1H), 8.46 (s, 1H), 8.21-8.10 (m, 2H), 7.94-7.76 (m, 4H), 7.48-7.41 (m, 1H), 4.29-4.11 (m, 3H), 3.41 (s, 3H), 3.21 (s, 1H), 2.07-1.97 (m, 1H), 1.93-1.81 (m, 2H), 1.81-1.75 (m, 1H), 1.71-1.56 (m, 2H).

### Example 398: Methyl 2-{6-cyclopropyl-4-[4-fluoro-2-(4-methyl-1,2,4-triazol-3-yl)phenyl]pyridin-2-yl}-7-methyl-1,3-benzoxazole-5-carboxylate (AES-3)

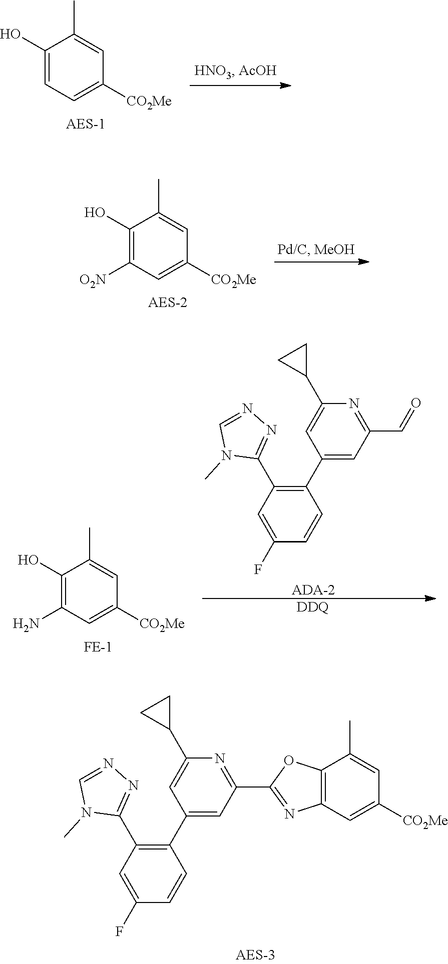

Step 1: Methyl 4-hydroxy-3-methyl-5-nitrobenzoate (AES-2). To a stirred solution of methyl 4-hydroxy-3-methylbenzoate (AES-1) (5.00 g, 1 Eq, 30.1 mmol) in AcOH (80 mL) was added HNO₃ (2.3 g, 1.2 Eq, 36.1 mmol) in portions at 0° C. The resulting mixture was stirred for 2 h at rt. The reaction was then quenched by the addition of 200 mL of ice water at 0° C. The precipitated solids were collected by filtration and washed with water (3×30 mL). This resulted in the sub-title compound (AES-2) (4.5 g, 21.3 mmol, 71%, 85% Purity) as a yellow solid. m/z 212.1 (M+H)⁺ (ES+).

Step 2: methyl 3-amino-4-hydroxy-5-methylbenzoate (FE-1). To a solution of the product from step 1 above (AES-2) (2.00 g, 1 Eq, 9.47 mmol) in MeOH (20 mL) was added Pd/C type 87L (2.00 g, 10% Wt, 10 Eq, 94.7 mmol) at rt under nitrogen atmosphere. The resulting mixture was stirred for 2 h at rt under hydrogen atmosphere. The resulting mixture was filtered; the filter cake was washed with MeOH (3×5 mL). The filtrate was concentrated in vacuo. This resulted in the sub-title compound (FE-1) (1.5 g, 8.28 mmol, 87%, 89% Purity) as a yellow solid. m/z 182.1 (M+H)⁺ (ES+).

Step 3: Methyl 2-{6-cyclopropyl-4-[4-fluoro-2-(4-methyl-1,2,4-triazol-3-yl)phenyl]pyridin-2-yl}-7-methyl-1,3-benzoxazole-5-carboxylate (AES-3). To a stirred solution of intermediate (ADA-2) (50 mg, 1 Eq, 0.16 mmol) and the product from step 2 above (FE-1) (42 mg, 1 Eq, 0.23 mmol) in DCM (5 mL) at rt. The resulting mixture was stirred for 2 h at 60° C. The mixture was cooled to rt. To the above mixture was added DDQ (70 mg, 2 Eq, 0.31 mmol) at rt. The resulting mixture was stirred for 2 h at rt. The resulting mixture was diluted with water and extracted with EtOAc (3×80 mL). The combined organic layers were washed with brine (2×80 mL), dried (Na₂SO₄). After filtration, the filtrate was concentrated in vacuo. The residue was purified by Prep-TLC with DCM/MeOH (12/1). The crude product was purified by Prep-HPLC with the following conditions (Column: XBridge Prep OBD C18 Column, 30*150 mm, 5 μm; Mobile Phase A: water (0.1% NH₄HCO₃+0.1% NH₃·H₂O), Mobile Phase B: MeCN; Flow rate: 60 mL/min; Gradient: 23% B to 50% B in 8 min; Wave Length: 254/220 nm; RT: 7.96) to afford the title compound (AES-3) (7.2 mg, 15 μmol, 9.6%, 99.9% Purity) as a white solid. m/z 484.0 (M+H)⁺ (ES+). ¹H NMR (300 MHz, DMSO-d6) δ 8.51 (s, 1H), 8.23-8.20 (m, 1H), 7.97-7.94 (m, 1H), 7.91-7.84 (m, 1H), 7.78 (d, J=1.5 Hz, 1H), 7.69-7.60 (m, 2H), 7.13 (d, J=1.6 Hz, 1H), 3.90 (s, 3H), 3.31 (s, 3H), 2.62 (t, J=0.8 Hz, 3H), 2.23-2.16 (m, 1H), 1.09-1.02 (m, 2H), 0.98-0.92 (m, 2H).

### Example 399: (2-{6-Cyclopropyl-4-[4-fluoro-2-(4-methyl-1,2,4-triazol-3-yl)phenyl]pyridin-2-yl}-7-methyl-1,3-benzoxazol-5-yl)methanol (AET-1)

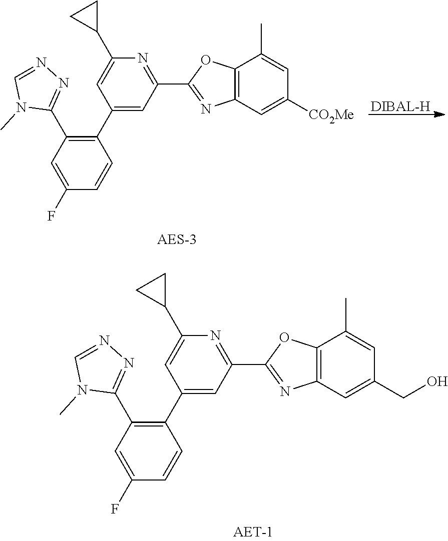 [SMILES: COC(=O)c1cc(C)c2oc(-c3cc(-c4ccc(F)cc4-c4nncn4C)cc(C4CC4)n3)nc2c1 ; "DIBAL-H" ; Cc1cc(CO)cc2nc(-c3cc(-c4ccc(F)cc4-c4nncn4C)cc(C4CC4)n3)oc12]

To a stirred solution of compound (AES-3) (22 mg, 1 Eq, 46 μmol) in THF (1 mL) was added DIBAL-H in THF (0.14 mL, 1 M, 3 Eq, 0.14 mmol) was stirred for 2 h at 0° C. under nitrogen atmosphere. The reaction was then quenched by the addition of 5 mL of ice water at 0° C. The resulting mixture was diluted with water and extracted with EtOAc (3×20 mL). The combined organic layers were washed with brine (2×20 mL), dried (Na₂SO₄). After filtration, the filtrate was concentrated in vacuo. The residue was purified by Prep-TLC with DCM/MeOH (10/1). The crude product was purified by Prep-HPLC with the following conditions (Column: XBridge Prep OBD C18 Column, 30*150 mm, 5 μm; Mobile Phase A: water (0.1% NH₄HCO₃+0.1% NH₃·H₂O), Mobile Phase B: MeCN; Flow rate: 60 mL/min; Gradient: 46% B to 56% B in 8 min; Wave Length: 254/220 nm; RT: 6.97) This resulted in the title compound (AET-1) (6.3 mg, 14 μmol, 30%, 99.9% Purity) as a white solid. m/z 456.0 (M+H)⁺ (ES+). ¹H NMR (300 MHz, DMSO-d6) δ 8.51 (s, 1H), 7.91-7.82 (m, 1H), 7.76 (d, J=1.6 Hz, 1H), 7.68-7.55 (m, 3H), 7.27-7.24 (m, 1H), 7.07 (d, J=1.6 Hz, 1H), 5.29 (t, J=5.7 Hz, 1H), 4.59 (d, J=5.7 Hz, 2H), 3.29 (s, 3H), 2.55 (s, 3H), 2.23-2.14 (m, 1H), 1.09-1.00 (m, 2H), 0.95-0.85 (m, 2H).

### Example 400: (1S,2R)-2-{[(2-{6-Cyclopropyl-4-[4-fluoro-2-(4-methyl-1,2,4-triazol-3-yl)phenyl]pyridin-2-yl}-7-methyl-1,3-benzoxazol-5-yl)methyl]amino}cyclopentan-1-ol (AEU-2)

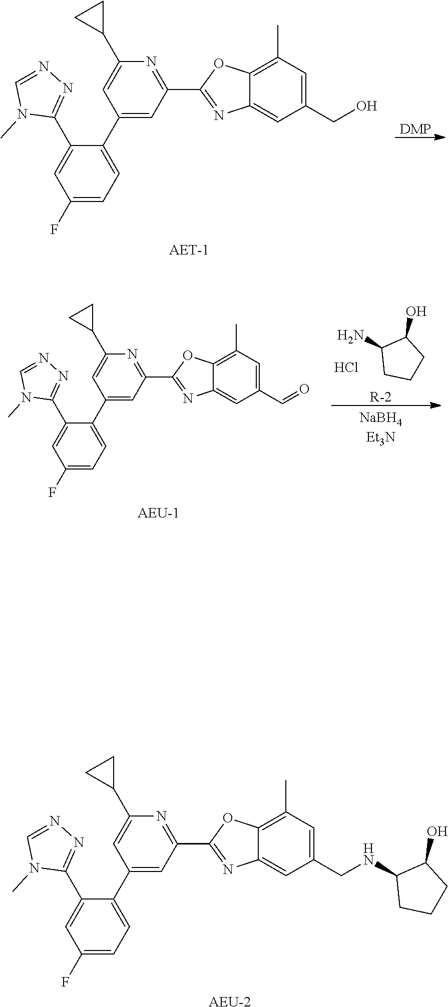

Step 5: 2-{6-Cyclopropyl-4-[4-fluoro-2-(4-methyl-1,2,4-triazol-3-yl)phenyl]pyridin-2-yl}-7-methyl-1,3-benzoxazole-5-carbaldehyde (AEU-1). To a stirred solution of compound (AET-1) (100 mg, 1 Eq, 0.22 mmol) in DCM (5 mL) was added DMP (121 mg, 1.3 Eq, 0.29 mmol) at rt. The resulting mixture was stirred for 1 h at rt. The resulting mixture was filtered the filter cake was washed with EtOAc (3×5 mL). The filtrate was concentrated in vacuo. This resulted in the sub-title compound (AEU-1) (90 mg, 0.20 mmol, 90%, 88% Purity) as a white solid. m/z 454.2 (M+H)⁺ (ES+)

Step 6: (1S,2R)-2-{[(2-{6-Cyclopropyl-4-[4-fluoro-2-(4-methyl-1,2,4-triazol-3-yl)phenyl]pyridin-2-yl}-7-methyl-1,3-benzoxazol-5-yl)methyl]amino}cyclopentan-1-ol (AEU-2). To a stirred solution of the product from step 1 above (AEU-1) (30 mg, 1 Eq, 66 μmol) and (1S,2R)-2-aminocyclopentan-1-ol (R-2) (7 mg, 1 Eq, 66 μmol) in MeOH (5 mL) was added Et₃N (33 mg, 5 Eq, 0.33 mmol) for 2 h at 60° C. under nitrogen atmosphere. The mixture was cooled to rt. To the above mixture was added NaBH₄ (25 mg, 10 Eq, 0.66 mmol) at 0° C. The resulting mixture was stirred for 2 h at rt. The reaction was then quenched by the addition of 2 mL of MeOH at 0° C. The resulting mixture was diluted with water and extracted with DCM (3×10 mL). The combined organic layers were washed with brine (2×10 mL), dried (Na₂SO₄). After filtration, the filtrate was concentrated in vacuo. The crude product was purified by Prep-HPLC with the following conditions: Column: XBridge Prep OBD C18 Column, 30×150 mm, 5 μm; Mobile Phase A: water (0.1% NH₄HCO₃+0.1% NH₃·H₂O) Mobile Phase B: MeCN Flow rate: 60 mL/min; Gradient: 30 B to 45 B in 9 min; Detector, UV 254/210 nm; RT: 8.3. The product-containing fractions were combined and evaporated partially in vacuo and lyophilized overnight to afford the title compound (AEU-2) (2.3 mg, 4.3 μmol, 6.5%, 99.9% Purity) as a white solid. m/z 539.3 (M+H)⁺ (ES+). ¹H NMR (400 MHz, Methanol-d4) δ 8.46 (s, 1H), 7.85-7.82 (m, 2H), 7.62-7.55 (m, 2H), 7.53-7.49 (m, 1H), 7.32 (s, 1H), 7.10 (d, J=1.6 Hz, 1H), 4.22-4.15 (m, 1H), 3.93 (d, J=21.0 Hz, 2H), 3.30-3.29 (m, 3H) 3.02-2.95 (m, 1H), 2.61 (s, 3H), 2.20-2.14 (m, 1H), 1.92-1.72 (m, 4H), 1.60-1.50 (m, 2H), 1.11-1.00 (m, 4H).

### Example 401: 1-({[(2-{6-Cyclopropyl-4-[4-fluoro-2-(4-methyl-1,2,4-triazol-3-yl)phenyl]pyridin-2-yl}-7-methyl-1,3-benzoxazol-5-yl)methyl]amino}methyl)cyclobutan-1-ol (AEV-1)

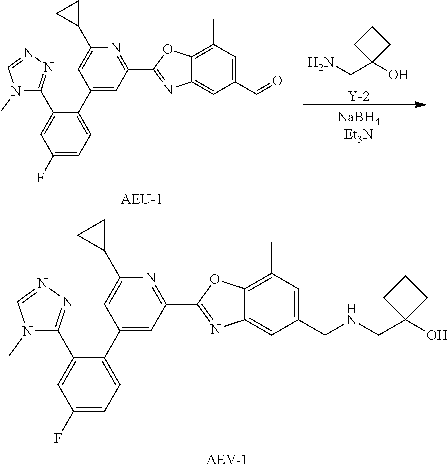

To a stirred solution of intermediate (AEU-1) (30 mg, 1 Eq, 66 μmol) and 1-(aminomethyl)cyclobutan-1-ol (Y-2) (8 mg, 1.2 Eq, 82 μmol) in MeOH (5 mL) was added Et₃N (34 mg, 5 Eq, 0.34 mmol) for 2 h at 60° C. under nitrogen atmosphere. The mixture was cooled to rt. To the above mixture was added NaBH₄ (25 mg, 10 Eq, 0.66 mmol) at 0° C. The resulting mixture was stirred for 1 h at rt. The reaction was then quenched by the addition of 2 mL of MeOH at 0° C. The resulting mixture was diluted with water and extracted with DCM (3×20 mL). The combined organic layers were washed with brine (2×20 mL), dried (Na₂SO₄). After filtration, the filtrate was concentrated in vacuo. The crude product was purified by Prep-HPLC with the following conditions: Column: XBridge Prep OBD C18 Column, 30×150 mm, 5 μm; Mobile Phase A: water (0.1% NH₄HCO₃+0.1% NH₃·H₂O) Mobile Phase B: MeCN; Flow rate: 60 mL/min; Gradient: 33 B to 45 B in 8 min; Detector, UV 254/210 nm; RT: 7.98. The product-containing fractions were combined and evaporated partially in vacuo and lyophilized overnight to afford the title compound (AEV-1) (4.2 mg, 7.8 μmol, 11%, 98.2% Purity) as a white solid. m/z 539.3 (M+H)⁺ (ES+). ¹H NMR (400 MHz, Methanol-d4) δ 8.46 (s, 1H), 7.87-7.82 (m, 2H), 7.62-7.49 (m, 3H), 7.32 (s, 1H), 7.11 (d, J=1.6 Hz, 1H), 3.98 (s, 2H), 3.31-3.30 (m, 3H), 2.77 (s, 2H), 2.62 (s, 3H), 2.18 (d, J=6.4 Hz, 1H), 2.10-2.02 (m, 4H), 1.77-1.71 (m, 1H), 1.54-1.48 (m, 1H), 1.10-1.03 (m, 4H).

### Example 402: (1S, 2R)-2-{1[(2-{6-Cyclopropyl-4-[4-fluoro-2-(4-methyl-1, 2, 4-triazol-3-yl) phenyl]pyridin-2-yl}-7-(trifluoromethyl)-1, 3-benzoxazol-5-yl) methyl](methyl)amino}cyclopentan-1-ol (AEW-1)

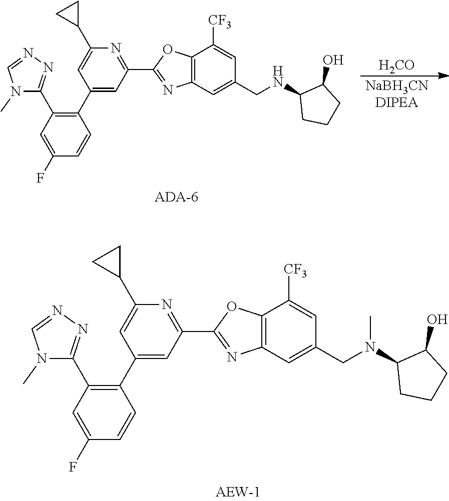 [SMILES: Cn1cnnc1-c1cc(F)ccc1-c1cc(-c2nc3cc(CN[C@@H]4CCC[C@@H]4O)cc(C(F)(F)F)c3o2)nc(C2CC2)c1 ; "H2CO" ; "NaBH3CNDIPEA" ; "ADA-6" ; CN(Cc1cc(C(F)(F)F)c2oc(-c3cc(-c4ccc(F)cc4-c4nncn4C)cc(C4CC4)n3)nc2c1)[C@@H]1CCC[C@@H]1O]

To a stirred solution of compound (ADA-6) (50 mg, 1 Eq, 84 μmol) and formaldehyde (13 mg, 5 Eq, 0.42 mmol) in MeOH (6 mL) was added DIPEA (65 mg, 6 Eq, 0.50 mmol) at rt. The resulting mixture was stirred for 3 h at 60° C. The mixture was allowed to cool down to rt. To the above mixture was added NaBH₃CN (27 mg, 5 Eq, 0.42 mmol) at rt. The resulting mixture was stirred for additional 3 h at rt. The reaction was then quenched by the addition of 2 mL of MeOH at 0° C. The resulting mixture was concentrated in vacuo. The crude product was purified by Prep-HPLC with the following conditions (Column: XBridge Prep OBD C18 Column, 30*150 mm, 5 μm; Mobile Phase A: water (0.1% NH₄HCO₃+0.1% NH₃·H₂O), Mobile Phase B: MeCN; Flow rate: 60 mL/min; Gradient: 50% B to 65% B in 9 min; Wave Length: 254/220 nm) to afford the title compound (AEW-1) (4.9 mg, 8.1 μmol, 9.5%, 99.3% Purity) as a white solid. m/z 607.1 (M+H)⁺ (ES+). ¹H NMR (400 MHz, DMSO-d6) δ 8.49 (s, 1H), 8.16 (s, 1H), 7.88-7.82 (m, 2H), 7.71 (d, J=1.5 Hz, 1H), 7.69-7.61 (m, 2H), 7.18 (d, J=1.6 Hz, 1H), 4.13 (d, J=3.9 Hz, 1H), 4.02 (d, J=3.1 Hz, 1H), 3.82 (d, J=13.7 Hz, 1H), 3.69 (d, J=13.6 Hz, 1H), 3.34 (s, 3H), 2.46 (d, J=3.6 Hz, 1H), 2.25-2.15 (m, 1H), 2.12 (s, 3H), 1.80 (t, J=9.4 Hz, 2H), 1.73-1.63 (m, 2H), 1.62-1.50 (m, 2H), 1.10-1.01 (m, 2H), 0.98-0.90 (m, 2H).

### Example 403: (2-{6-Cyclopropyl-4-[4-fluoro-2-(4-methyl-1,2,4-triazol-3-yl)phenyl]pyridin-2-yl}-7-methoxy-1,3-benzoxazol-5-yl)methanol (AEX-2)

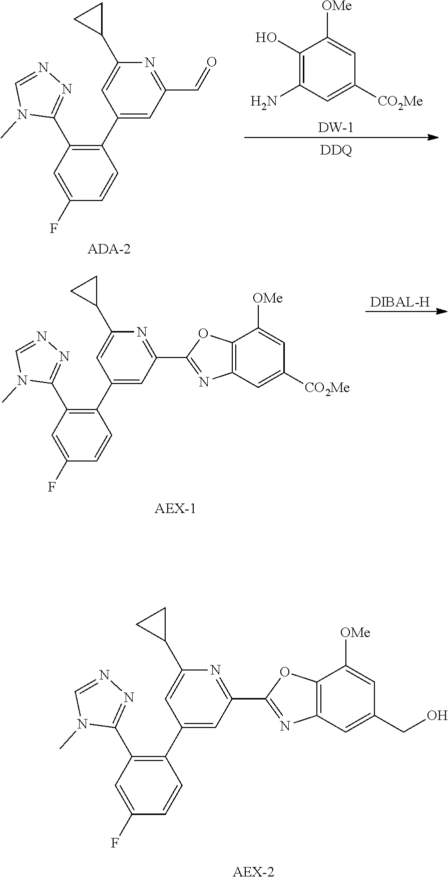

Step 1: Methyl 2-{6-cyclopropyl-4-[4-fluoro-2-(4-methyl-1,2,4-triazol-3-yl)phenyl]pyridin-2-yl}-7-methoxy-1,3-benzoxazole-5-carboxylate (AEX-1). To a stirred mixture of intermediate (ADA-2) (100 mg, 1 Eq, 0.31 mmol) in DCM (5 mL) was added methyl 3-amino-4-hydroxy-5-methoxybenzoate (DW-1) (92 mg, 1.5 Eq, 0.47 mmol) at rt. The resulting mixture was stirred for 1 h at 60° C. The mixture was cooled to rt. To the above mixture was added DDQ (106 mg, 1.5 Eq, 0.47 mmol) at rt. The resulting mixture was stirred for additional 1 h at rt. The resulting mixture was diluted with water and extracted with EtOAc (3×20 mL). The combined organic layers were washed with brine (2×20 mL), dried (Na₂SO₄). After filtration, the filtrate was concentrated in vacuo. The residue was purified by Prep-TLC with DCM/MeOH (12/1) to afford the sub-title compound (AEX-1) (75 mg, 0.15 mmol, 48%, 95% Purity) as a brown solid. m/z 500.2 (M+H)⁺ (ES+).

Step 2: (2-{6-Cyclopropyl-4-[4-fluoro-2-(4-methyl-1,2,4-triazol-3-yl)phenyl]pyridin-2-yl}-7-methoxy-1,3-benzoxazol-5-yl)methanol (AEX-2). To a stirred mixture of the product from step 1 above (AEX-1) (20 mg, 1 Eq, 0.04 mmol) in THF (2 mL) was added DIBAL-H (g) in THF (0.4 mL, 1 M, 10 Eq, 0.40 mmol) at 0° C. under nitrogen atmosphere. The resulting mixture was stirred for 3 h at 0° C. under nitrogen atmosphere. The reaction was then quenched by the addition of 2 mL of ice water at 0° C. The resulting mixture was diluted with water and extracted with DCM (3×10 mL). The combined organic layers were washed with brine (2×10 mL), dried (Na₂SO₄). After filtration, the filtrate was concentrated in vacuo. The crude product was purified by Prep-HPLC with the following conditions (Column: Sunfire prep C18 column, 30*150 mm, 5 μm; Mobile Phase A: water (0.1% FA), Mobile Phase B: MeCN; Flow rate: 60 mL/min; Gradient: 25% B to 50% B in 7 min; Wave Length: 254/220 nm; RT: 6.6) to afford the title compound (AEX-2) (2.0 mg, 4.2 μmol, 11%, 99.7% Purity) as a white solid. m/z 472.0 (M+H)⁺ (ES+). ¹H NMR (300 MHz, DMSO-d6) δ 8.49 (s, 1H), 7.91-7.82 (m, 1H), 7.79 (d, J=1.5 Hz, 1H), 7.68-7.57 (m, 2H), 7.33 (s, 1H), 7.12-7.01 (m, 2H), 5.34 (t, J=5.7 Hz, 1H), 4.61 (d, J=5.5 Hz, 2H), 4.01 (s, 3H), 3.29 (s, 3H), 2.22-2.13 (m, 1H), 1.11-1.09 (m, 4H).

### Example 404: [(2-{6-Cyclopropyl-4-[4-fluoro-2-(4-methyl-1,2,4-triazol-3-yl)phenyl]pyridin-2-yl}-1,3-benzoxazol-5-yl)methyl](cyclopropylmethyl)amine (AEY-2)

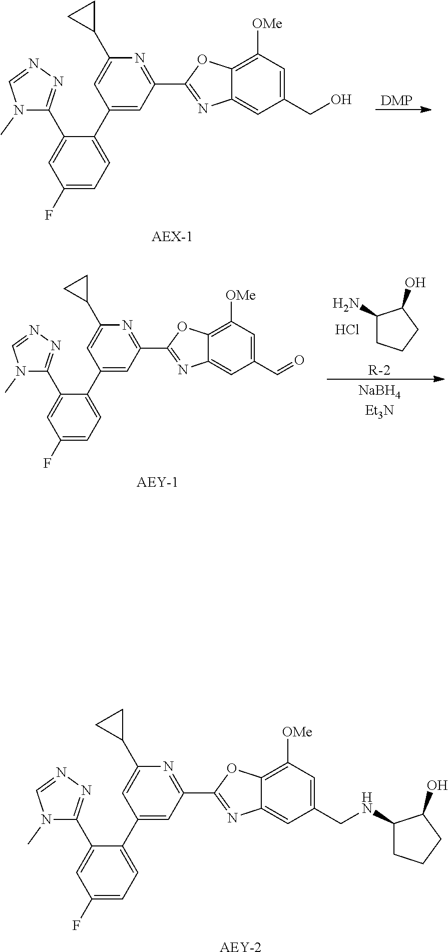

Step 1: 2-{6-Cyclopropyl-4-[4-fluoro-2-(4-methyl-1,2,4-triazol-3-yl)phenyl]pyridin-2-yl}-7-methoxy-1,3-benzoxazole-5-carbaldehyde (AEY-1). To a stirred solution of compound (AEX-2) (200 mg, 1 Eq, 0.42 mmol) in DCM (15 mL) was added DMP (234 mg, 1.3 Eq, 0.55 mmol) at rt under nitrogen atmosphere. The resulting mixture was stirred for 1 h at rt under nitrogen atmosphere. The resulting mixture was concentrated in vacuo. The residue was purified by Prep-TLC with DCM/MeOH (15/1) to afford the sub-title compound (AEY-1) (163 mg, 0.35 mmol, 75%, 92% Purity) as an off-white solid. m/z 470.2 (M+H)⁺ (ES+)

Step 2: [(2-{6-Cyclopropyl-4-[4-fluoro-2-(4-methyl-1,2,4-triazol-3-yl)phenyl]pyridin-2-yl}-1,3-benzoxazol-5-yl)methyl](cyclopropylmethyl)amine (AEY-2). To a stirred mixture of the product from step 1 above (AEY-1) (30 mg, 1 Eq, 68 μmol) and (1S,2R)-2-aminocyclopentan-1-ol, HCl (R-2) (7 mg, 1.5 Eq, 0.10 mmol) in MeOH (5 mL) were added Et₃N (21 mg, 3 Eq, 0.20 mmol) at rt. The resulting mixture was stirred for 1 h at 60° C. The mixture was cooled to rt. The mixture was allowed to cool down to rt. To the above mixture was added NaBH₄ (13 mg, 5 Eq, 0.34 mmol) at 0° C. The resulting mixture was stirred for 1 h at rt. The reaction was then quenched by the addition of 2 mL of MeOH at 0° C. The resulting mixture was concentrated in vacuo. The residue was purified by Prep-TLC with DCM/MeOH (10/1). The crude product was purified by Prep-HPLC with the following conditions (Column: Xselect CSH C18 OBD Column 30*150 mm, 5 m, n; Mobile Phase A: water (0.1% FA), Mobile Phase B: MeCN; Flow rate: 60 mL/min; Gradient: 8% B to 32% B in 7 min; Wave Length: 254/220 nm; RT: 6.5) to afford the title compound (AEY-2) (19.4 mg, 35 μmol, 57%, 99.6% Purity) as a white solid. m/z 555.2 (M+H)⁺ (ES+). ¹H NMR (300 MHz, DMSO-d6) δ 8.49 (s, 1H), 7.88-7.75 (m, 2H), 7.67-7.57 (m, 2H), 7.37 (d, J=1.3 Hz, 1H), 7.15-7.05 (m, 2H), 4.01 (s, 4H), 3.84 (d, J=6.4 Hz, 2H), 3.30 (s, 3H), 2.87-2.76 (m, 1H), 2.21-2.13 (m, 1H), 1.74-1.58 (m, 4H), 1.49-1.38 (m, 2H), 1.08-1.00 (m, 2H), 0.95-0.88 (m, 2H).

### Example 405: [(2-{6-Cyclopropyl-4-[4-fluoro-2-(4-methyl-1,2,4-triazol-3-yl)phenyl]pyridin-2-yl}-7-methyl-1,3-benzoxazol-5-yl)methyl](2-methoxyethyl)amine (AEZ-1)

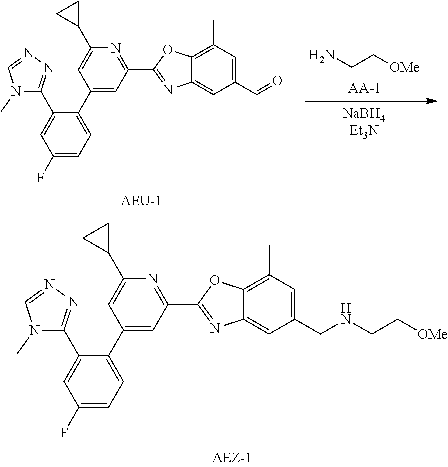

To a stirred solution of intermediate (AEU-1) (30 mg, 1 Eq, 66 μmol) and 2-methoxyethan-1-amine (AA-1) (5 mg, 1 Eq, 66 μmol) in MeOH (5 mL) was added Et₃N (34 mg, 5 Eq, 0.34 mmol) for 2 h at 60° C. under nitrogen atmosphere. The mixture was allowed to cool down to rt. To the above mixture was added NaBH₄ (25 mg, 10 Eq, 0.66 mmol) at 0° C. The resulting mixture was stirred for 1 h at rt. The reaction was then quenched by the addition of 2 mL of MeOH at 0° C. The resulting mixture was diluted with water and extracted with DCM (3×10 mL). The combined organic layers were washed with brine (2×10 mL), dried (Na₂SO₄). After filtration, the filtrate was concentrated in vacuo. The crude product was purified by Prep-HPLC with the following conditions: Column: XBridge Prep OBD C18 Column, 30×150 mm, 5 μm; Mobile Phase A: water (0.1% NH₄HCO₃+0.1% NH₃·H₂O) Mobile Phase B: MeCN; Flow rate: 60 mL/min; Gradient: 30 B to 41 B in 9 min; Detector, UV 254/210 nm; RT: 8.68. The product-containing fractions were combined and evaporated partially in vacuo and lyophilized overnight to afford the title compound (AEZ-1) (1.1 mg, 2.1 μmol, 3.2%, 99.6% Purity) as a white solid. m/z 513.4 (M+H)⁺ (ES+). ¹H NMR (400 MHz, Methanol-d4) δ 8.46 (s, 1H), 7.91-7.80 (m, 2H), 7.62-7.52 (m, 2H), 7.54-7.45 (m, 1H), 7.28 (s, 1H), 7.09 (d, J=1.6 Hz, 1H), 3.88 (s, 2H), 3.52 (t, J=5.4 Hz, 3H), 3.32-3.28 (m, 5H), 2.78 (t, J=5.4 Hz, 2H), 2.61 (s, 3H), 2.21-2.14 (m, 1H), 1.17-0.98 (m, 4H).

### Example 406: (2-{6-Cyclopropyl-4-[4-fluoro-2-(4-methyl-1,2,4-triazol-3-yl)phenyl]pyridin-2-yl}-7-fluoro-1,3-benzoxazol-5-yl)methanol (AFA-2)

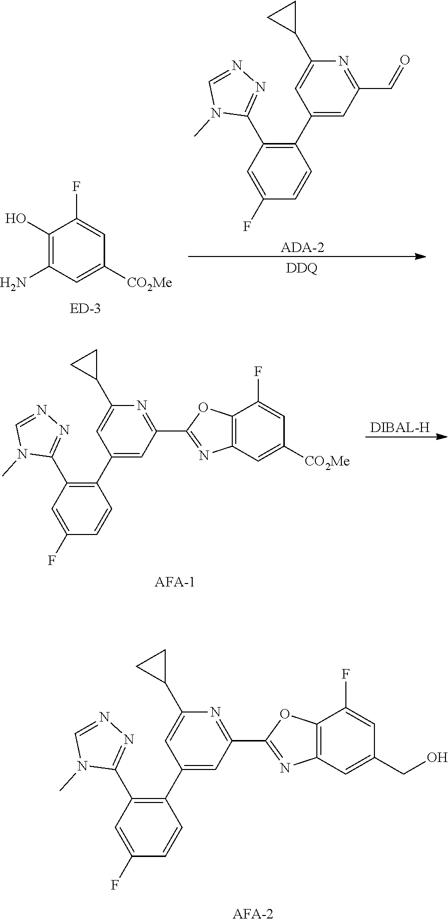

Step 1: Methyl 2-{6-cyclopropyl-4-[4-fluoro-2-(4-methyl-1,2,4-triazol-3-yl)phenyl]pyridin-2-yl}-7-fluoro-1,3-benzoxazole-5-carboxylate (AFA-1). A solution of intermediate (ADA-2) (500 mg, 1 Eq, 1.55 mmol) in DCM (20 mL) was treated with intermediate (ED-3) (473 mg, 1.5 Eq, 2.33 mmol) for 1 h at 60° C. under nitrogen atmosphere. The mixture was allowed to cool down to rt. To the above mixture was added DDQ (704 mg, 2 Eq, 3.10 mmol) at rt. The resulting mixture was stirred for 2 h at rt. The resulting mixture was diluted with water and extracted with EtOAc (3×80 mL). The combined organic layers were washed with brine (2×80 mL), dried (Na₂SO₄). After filtration, the filtrate was concentrated in vacuo. The crude product was purified by reverse flash column chromatography with the following conditions: Column, C18; mobile phase, water (0.1% FA) and MeCN (10% MeCN up to 50% in 20 min); Detector, UV 254/220 nm. This resulted in the sub-title compound (AFA-1) (400 mg, 0.82 mmol, 66%, 90% Purity) as a yellow solid. m/z 488.1 (M+H)⁺ (ES+).

Step 2: (2-{6-Cyclopropyl-4-[4-fluoro-2-(4-methyl-1,2,4-triazol-3-yl)phenyl]pyridin-2-yl}-7-fluoro-1,3-benzoxazol-5-yl)methanol (AFA-2). To a stirred solution of the product from step 1 above (AFA-1) (50 mg, 1 Eq, 0.10 mmol) in THF (5 mL) was added DIBAL-H (g) in THF (0.8 mL, 1 M, 8 Eq, 0.8 mmol) was stirred for 2 h at 0° C. under nitrogen atmosphere. The reaction was then quenched by the addition of 3 mL of ice water at 0° C. The resulting mixture was diluted with water and extracted with EtOAc (3×20 mL). The combined organic layers were washed with brine (2×20 mL), dried (Na₂SO₄). After filtration, the filtrate was concentrated in vacuo. The residue was purified by Prep-TLC with DCM/MeOH (10/1). The crude product was purified by Prep-HPLC with the following conditions: Column: XBridge Prep OBD C18 Column, 30×150 mm, 5 μm; Mobile Phase A: water (0.1% NHLHCO₃+0.1% NH₃·H₂O) Mobile Phase B: MeCN; Flow rate: 60 mL/min; Gradient: 48 B to 58 B in 10 min; Detector, UV 254/210 nm; RT: 9.8. The product-containing fractions were combined and evaporated partially in vacuo and lyophilized overnight to afford the title compound (AFA-2) (6.4 mg, 14 μmol, 13%, 98.9% Purity) as a white solid. m/z 460.0 (M+H)⁺ (ES+). ¹H NMR (400 MHz, Methanol-d4) δ 8.47 (s, 1H), 7.91 (d, J=1.5 Hz, 1H), 7.86-7.79 (m, 1H), 7.62-7.54 (m, 2H), 7.53-7.46 (m, 1H), 7.31-7.26 (m, 1H), 7.09 (d, J=1.6 Hz, 1H), 4.72 (s, 2H), 3.31 (s, 3H), 2.16 (m, 1H), 1.13-0.98 (m, 4H).

### Example 407: (1S,2R)-2-{[(2-{6-Cyclopropyl-4-[4-fluoro-2-(4-methyl-1,2,4-triazol-3-yl)phenyl]pyridin-2-yl}-7-fluoro-1,3-benzoxazol-5-yl)methyl]amino}cyclopentan-1-ol (AFB-2)

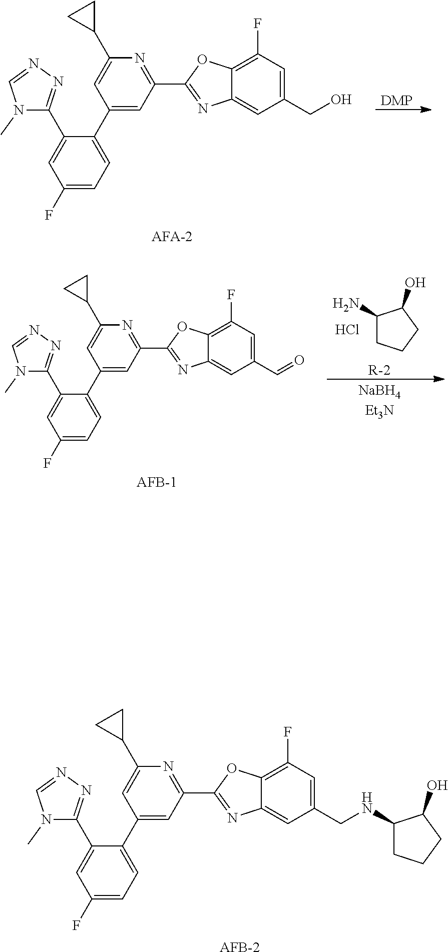

Step 1: 2-{6-cyclopropyl-4-[4-fluoro-2-(4-methyl-1,2,4-triazol-3-yl)phenyl]pyridin-2-yl}-7-fluoro-1,3-benzoxazole-5-carbaldehyde (AFB-1). To a stirred solution of compound (AFA-2) (300 mg, 1 Eq, 0.65 mmol) in DCM (5 mL) was added DMP (360 mg, 1.3 Eq, 0.85 mmol) at rt. The resulting mixture was stirred for 1 h at rt. The resulting mixture was filtered the filter cake was washed with EtOAc (3×10 mL). The filtrate was concentrated in vacuo. This resulted in the sub-title compound (AFB-1) (280 mg, 0.61 mmol, 94%, 92% Purity) as a yellow solid. m/z 458.1 (M+H)⁺ (ES+).

Step 2: (1S,2R)-2-{[(2-{6-Cyclopropyl-4-[4-fluoro-2-(4-methyl-1,2,4-triazol-3-yl)phenyl]pyridin-2-yl}-7-fluoro-1,3-benzoxazol-5-yl)methyl]amino}cyclopentan-1-ol (AFB-2). To a stirred solution of the product from step 1 above (AFB-1) (30 mg, 1 Eq, 66 μmol) and (1S,2R)-2-aminocyclopentan-1-ol (R-2) (13 mg, 2 Eq, 0.13 mmol) in MeOH (3 mL) was added Et₃N (33 mg, 5 Eq, 0.33 mmol) for 2 h at 60° C. under nitrogen atmosphere. The mixture was allowed to cool down to rt. To the above mixture was added NaBH₄ (25 mg, 10 Eq, 0.66 mmol) at 0° C. The resulting mixture was stirred for 2 h at rt. The reaction was then quenched by the addition of 2 mL of MeOH at 0° C. The resulting mixture was diluted with water and extracted with DCM (3×20 mL). The combined organic layers were washed with brine (2×20 mL), dried (Na₂SO₄). After filtration, the filtrate was concentrated in vacuo. The crude product was purified by Prep-HPLC with the following conditions: Column: XBridge Prep OBD C18 Column, 30×150 mm, 5 μm; Mobile Phase A: water (0.1% NH₄HCO₃+0.1% NH₃·H₂O) Mobile Phase B: MeCN; Flow rate: 60 mL/min; Gradient: 36 B to 46 B in 8 min; Detector, UV 254/210 nm; RT: 7.9. The product-containing fractions were combined and evaporated partially in vacuo and lyophilized overnight to afford the sub-title compound (AFB-2) (3.5 mg, 6.5 μmol, 9.8%, 99.4% Purity) as a white solid. m/z 543.3 (M+H)⁺ (ES+). ¹H NMR (400 MHz, Methanol-d4) δ 8.46 (s, 1H), 7.91 (d, J=1.6 Hz, 1H), 7.87-7.81 (m, 1H), 7.63-7.54 (m, 2H), 7.53-7.49 (m, 1H), 7.38-7.32 (m, 1H), 7.09 (d, J=1.6 Hz, 1H), 4.19-4.10 (m, 1H), 4.00-3.85 (m, 2H), 3.32-3.31 (m, 3H), 3.00-2.89 (m, 1H), 2.23-2.10 (m, 1H), 2.07-1.89 (m, 1H), 1.84-1.75 (m, 2H), 1.75-1.68 (m, 1H), 1.61-1.49 (m, 2H), 1.10-1.03 (m, 4H).

### Example 408: 1-({[(2-{6-Cyclopropyl-4-[4-fluoro-2-(4-methyl-1,2,4-triazol-3-yl)phenyl]pyridin-2-yl}-1,3-benzoxazol-5-yl)methyl]amino}methyl)cyclobutan-1-ol (AFC-1)

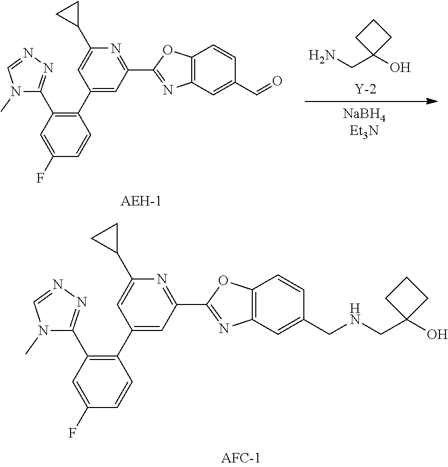

To a stirred mixture of intermediate (AEH-1) (22 mg, 1 Eq, 0.05 mmol) and 1-(aminomethyl)cyclobutan-1-ol (Y-2) (8 mg, 1.5 Eq, 75 μmol) in MeOH (2 mL) was added Et₃N (15 mg, 3 Eq, 0.15 mmol) at rt. The resulting mixture was stirred for 1 h at 60° C. The mixture was allowed to cool down to rt. To the above mixture was added NaBH₄ (9 mg, 5 Eq, 0.25 mmol) at rt. The resulting mixture was stirred for additional 1 h at rt. The reaction was then quenched by the addition of 2 mL of MeOH at 0° C. The resulting mixture was concentrated in vacuo. The residue was purified by Prep-TLC with DCM/MeOH (10/1). The crude product was purified by Prep-HPLC with the following conditions (Column: XBridge Prep OBD C18 Column, 30*150 mm, 5 μm; Mobile Phase A: water (0.1% NH₄HCO₃+0.1% NH₃·H₂O), Mobile Phase B: MeCN; Flow rate: 60 mL/min; Gradient: 32% B to 42% B in 9 min; Wave Length: 254/220 nm; RT: 8.35) to afford the title compound (AFC-1) (5.2 mg, 9.9 μmol, 20%, 99.3% Purity) as a white solid. m/z 525.2 (M+H)⁺ (ES+). ¹H NMR (400 MHz, DMSO-d6) δ 8.50 (s, 1H), 7.88-7.82 (m, 1H), 7.83-7.75 (m, 3H), 7.67-7.59 (m, 2H), 7.48-7.44 (m, 1H), 7.08 (d, J=1.6 Hz, 1H), 4.91 (s, 1H), 3.89 (s, 2H), 3.30 (s, 3H), 2.55 (s, 2H), 2.21-2.11 (m, 1H), 2.04-1.96 (m, 2H), 1.94-1.84 (m, 2H), 1.66-1.55 (m, 1H), 1.45-1.33 (m, 1H), 1.06-1.00 (m, 2H), 0.96-0.90 (m, 2H).

### Example 409: 1-{[(2-{6-Cyclopropyl-4-[4-fluoro-2-(4-methyl-1,2,4-triazol-3-yl)phenyl]pyridin-2-yl}-1,3-benzoxazol-5-yl)methyl]amino}-2-methylpropan-2-ol (AFD-1)

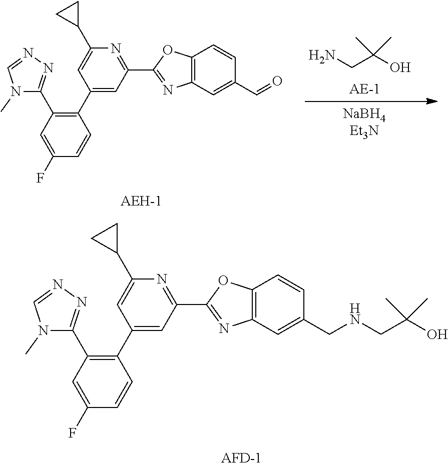

To a stirred mixture of intermediate (AEH-1) (30 mg, 1 Eq, 68 μmol) and 1-amino-2-methylpropan-2-ol (AE-1) (9 mg, 1.5 Eq, 0.10 mmol) in MeOH (3 mL) was added Et₃N (21 mg, 3 Eq, 0.20 mmol) at rt. The resulting mixture was stirred for 2 h at 60° C. under air atmosphere. The mixture was allowed to cool down to rt. To the above mixture was added NaBH₄ (13 mg, 5 Eq, 0.34 mmol) at rt. The resulting mixture was stirred for additional 1 h at rt. The reaction was then quenched by the addition of 2 mL of MeOH at 0° C. The resulting mixture was concentrated in vacuo. The residue was purified by Prep-TLC with DCM/MeOH (10/1). The crude product was purified by Prep-HPLC with the following conditions (Column: XBridge Prep OBD C18 Column, 30*150 mm, 5 μm; Mobile Phase A: water (0.1% NH₄HCO₃+0.1% NH₃·H₂O), Mobile Phase B: MeCN; Flow rate: 60 mL/min; Gradient: 33% B to 52% B in 8 min; Wave Length: 254/220 nm; RT: 6.63) to afford the title compound (AFD-1) (18.2 mg, 36 μmol, 52%, 95.7% Purity) as a white solid. m/z 513.2 (M+H)⁺ (ES+). ¹H NMR (300 MHz, DMSO-d6) δ 8.50 (s, 1H), 7.89-7.73 (m, 4H), 7.68-7.56 (m, 2H), 7.49-7.40 (m, 1H), 7.09 (d, J=1.6 Hz, 1H), 4.21 (s, 1H), 3.87 (s, 2H), 3.29 (s, 3H), 2.40 (s, 2H), 2.24-2.10 (m, 1H), 1.11 (s, 6H), 1.08-0.89 (m, 4H).

### Example 410: 1-({[(2-{6-Cyclopropyl-4-[4-fluoro-2-(4-methyl-1,2,4-triazol-3-yl)phenyl]pyridin-2-yl}-1,3-benzoxazol-5-yl)methyl]amino}methyl)cyclopentan-1-ol (AFE-1)

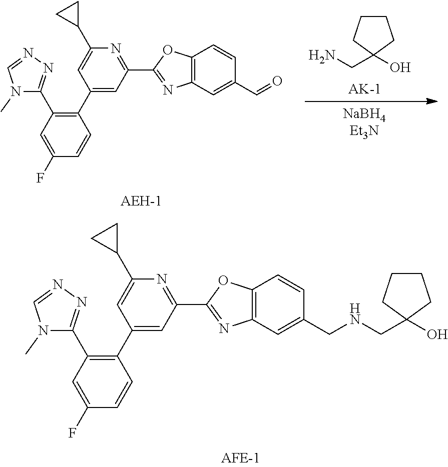

To a stirred mixture of intermediate (AEH-1) (30 mg, 1 Eq, 68 μmol) and 1-(aminomethyl)cyclopentan-1-ol (AK-1) (12 mg, 1.5 Eq, 0.10 mmol) in MeOH (3 mL) was added Et₃N (21 mg, 3 Eq, 0.20 mmol) at rt. The resulting mixture was stirred for 2 h at 60° C. The mixture was allowed to cool down to rt. To the above mixture was added NaBH₄ (13 mg, 5 Eq, 0.34 mmol) at rt. The resulting mixture was stirred for additional 1 h at rt. The reaction was then quenched by the addition of 2 mL of MeOH at 0° C. The resulting mixture was concentrated in vacuo. The residue was purified by Prep-TLC with DCM/MeOH (10/1). The crude product was purified by Prep-HPLC with the following conditions (Column: XBridge Prep OBD C18 Column, 30*150 mm, 5 μm; Mobile Phase A: water (0.1% NH₄HCO₃+0.1% NH₃·H₂O), Mobile Phase B: MeCN; Flow rate: 60 mL/min; Gradient: 36% B to 52% B in 8 min; Wave Length: 254/220 nm; RT: 7.52) to afford the title compound (AFE-1) (18.6 mg, 35 μmol, 51%, 98.8% Purity) as a white solid. m/z 539.2 (M+H)⁺ (ES+). ¹H NMR (300 MHz, DMSO-d6) δ 8.50 (s, 1H), 7.89-7.74 (m, 4H), 7.69-7.55 (m, 2H), 7.49-7.42 (m, 1H), 7.09 (d, J=1.6 Hz, 1H), 4.17 (s, 1H), 3.88 (s, 2H), 3.29 (s, 3H), 2.52 (s, 2H), 2.19-2.12 (m, 1H), 1.74-1.42 (m, 8H), 1.09-0.99 (m, 2H), 0.98-0.88 (m, 2H).

### Example 411: 1-Cyclobutyl-N-((2-(6-cyclopropyl-4-(4-fluoro-2-(4-methyl-4H-1,2,4-triazol-3-yl)phenyl)pyridin-2-yl)-7-methylbenzo[d]oxazol-5-yl)methyl)methanamine (AFF-1)

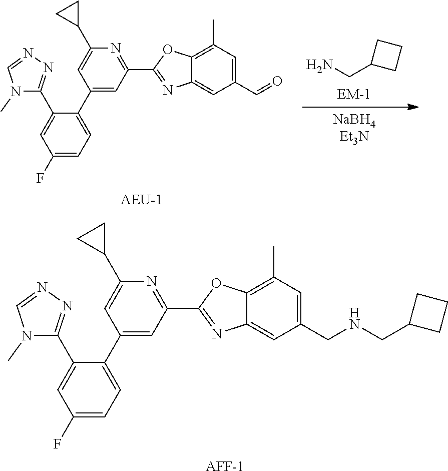

To a stirred mixture of intermediate (AEU-1) (20 mg, 1 Eq, 44 μmol) and 1-cyclobutylmethanamine (EM-1) (8 mg, 2 Eq, 88 μmol) in MeOH (2 mL) was added Et₃N (13 mg, 3 Eq, 0.13 mmol) at rt. The resulting mixture was stirred for 2 h at 60° C. The mixture was allowed to cool down to rt. To the above mixture was added NaBH₄ (8 mg, 5 Eq, 0.22 mmol) at rt. The resulting mixture was stirred for additional 2 h at rt. The reaction was then quenched by the addition of 2 mL of MeOH at 0° C. The resulting mixture was concentrated in vacuo. The residue was purified by Prep-TLC with DCM/MeOH (10/1). The crude product was purified by Prep-HPLC with the following conditions (Column: XBridge Prep OBD C18 Column, 30*150 mm, 5 μm; Mobile Phase A: water (0.1% NH₄HCO₃+0.1% NH₃·H₂O), Mobile Phase B: MeCN; Flow rate: 60 mL/min; Gradient: 43% B to 53% B in 8 min; Wave Length: 254/220 nm; RT: 7.67) to afford the title compound (AFF-1) (7.7 mg, 15 μmol, 33%, 98.1% Purity) as a white solid. m/z 523.2 (M+H)⁺ (ES+). ¹H NMR (300 MHz, DMSO-d6) δ 8.50 (s, 1H), 7.91-7.82 (m, 1H), 7.74 (d, J=1.5 Hz, 1H), 7.69-7.56 (m, 3H), 7.27 (s, 1H), 7.08 (d, J=1.6 Hz, 1H), 3.79 (s, 2H), 3.33 (s, 2H), 3.29 (s, 3H), 3.37 (s, 3H), 2.46-2.36 (m, 1H), 2.22-2.12 (m, 1H), 2.06-1.94 (m, 2H), 1.87-1.74 (m, 2H), 1.73-1.55 (m, 2H), 1.10-0.99 (m, 2H), 0.97-0.87 (m, 2H).

### Example 412: (1S,2R)-2-(((2-(6-Cyclopropyl-4-(4-fluoro-2-(4-methyl-4H-1,2,4-triazol-3-yl)phenyl)pyridin-2-yl)-7-methylbenzo[d]oxazol-5-yl)methyl)(methyl)amino)cyclopentan-1-ol (AFG-1)

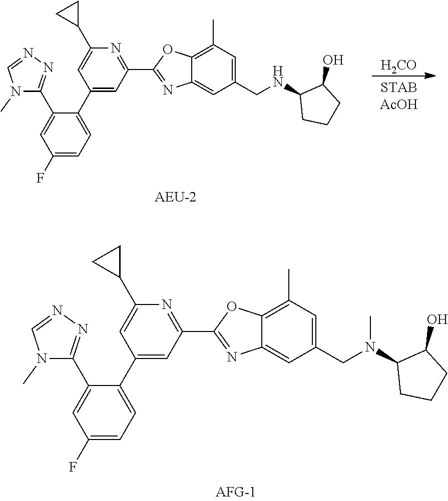 [SMILES: Cc1cc(CN[C@@H]2CCC[C@@H]2O)cc2nc(-c3cc(-c4ccc(F)cc4-c4nncn4C)cc(C4CC4)n3)oc12 ; "H2CO" ; Cc1cc(CN(C)[C@@H]2CCC[C@@H]2O)cc2nc(-c3cc(-c4ccc(F)cc4-c4nncn4C)cc(C4CC4)n3)oc12 ; "STABAcOH"]

To a stirred mixture of compound (AEU-2) (30 mg, 1 Eq, 56 μmol) in AcOH (3 mL) was added formaldehyde (0.2 mL) at rt. The resulting mixture was stirred for 10 min at rt. To the above mixture was added NaBH(OAc)₃ (118 mg, 10 Eq, 0.56 mmol) at rt. The resulting mixture was stirred for additional 2 h at rt. The reaction was then quenched by the addition of 2 mL of MeOH at 0° C. The resulting mixture was concentrated in vacuo. The residue was purified by Prep-TLC with DCM/MeOH (10/1). The crude product was purified by Prep-HPLC with the following conditions (Column: XBridge Prep OBD C18 Column, 30*150 mm, 5 μm; Mobile Phase A: water (0.1% NH₄HCO₃+0.1% NH₃·H₂O), Mobile Phase B: MeCN; Flow rate: 60 mL/min; Gradient: 42% B to 52% B in 9 min; Wave Length: 254/220 nm; RT: 8.68) to afford the title compound (AFG-1) (7.4 mg, 13 μmol, 24%, 99.7% Purity) as a white solid. m/z 553.2 (M+H)⁺ (ES+). ¹H NMR (300 MHz, DMSO-d6) δ 8.51 (s, 1H), 7.91-7.82 (m, 1H), 7.72 (d, J=1.6 Hz, 1H), 7.69-7.56 (m, 3H), 7.30 (s, 1H), 7.10 (d, J=1.6 Hz, 1H), 4.12 (d, J=3.9 Hz, 1H), 3.85-3.56 (m, 3H), 3.29 (s, 3H), 2.55 (s, 3H), 2.49 (s, 1H), 2.22-2.08 (m, 4H), 1.86-1.59 (m, 6H), 1.08-0.90 (m, 4H).

### Example 413: 1-({[(2-{6-Cyclopropyl-4-[4-fluoro-2-(4-methyl-1,2,4-triazol-3-yl)phenyl]pyridin-2-yl}-7-methyl-1,3-benzoxazol-5-yl)methyl](methyl)amino}methyl)cyclobutan-1-ol (AFH-2)

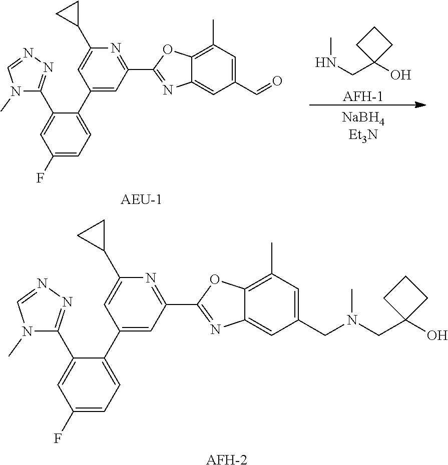

To a stirred mixture of intermediate (AEU-1) (30 mg, 1 Eq, 66 μmol) and 1-[(methylamino)methyl]cyclobutan-1-ol (AFH-1) (15 mg, 2 Eq, 0.13 mmol) in MeOH (2 mL) was added Et₃N (20 mg, 3 Eq, 0.20 mmol) at rt. The resulting mixture was stirred for 2 h at 60° C. The mixture was allowed to cool down to rt. To the above mixture was added NaBH₄ (13 mg, 5 Eq, 0.33 mmol) at rt. The resulting mixture was stirred for additional 1 h at rt. The reaction was then quenched by the addition of 2 mL of MeOH at 0° C. The resulting mixture was concentrated in vacuo. The residue was purified by Prep-TLC with DCM/MeOH (10/1). The crude product was purified by Prep-HPLC with the following conditions (Column: XBridge Prep OBD C18 Column, 30*150 mm, 5 μm; Mobile Phase A: water (0.1% NH₄HCO₃+0.1% NH₃·H₂O), Mobile Phase B: MeCN; Flow rate: 60 mL/min; Gradient: 45% B to 55% B in 8 min; Wave Length: 254/220 nm; RT: 7.77) to afford the title compound (AFH-2) (7.5 mg, 14 μmol, 21%, 99.5% Purity) as a white solid. m/z 553.2 (M+H)⁺ (ES+). ¹H NMR (300 MHz, DMSO-d6) δ 8.50 (s, 1H), 7.91-7.82 (m, 1H), 7.74 (d, J=1.5 Hz, 1H), 7.67-7.56 (m, 3H), 7.28 (s, 1H), 7.08 (d, J=1.6 Hz, 1H), 4.82 (s, 1H), 3.66 (s, 2H), 3.29 (s, 3H), 2.54 (s, 3H), 2.47 (s, 2H), 2.24 (s, 3H), 2.21-2.15 (m, 1H), 2.07-1.98 (m, 2H), 1.95-1.86 (m, 2H), 1.67-1.54 (m, 1H), 1.38-1.26 (m, 1H), 1.07-1.01 (m, 2H), 0.97-0.94 (m, 2H).

### Example 414: 1-({[(2-{6-Cyclopropyl-4-[4-fluoro-2-(4-methyl-1,2,4-triazol-3-yl)phenyl]pyridin-2-yl}-7-fluoro-1,3-benzoxazol-5-yl)methyl]amino}methyl)cyclobutan-1-ol (AFI-1)

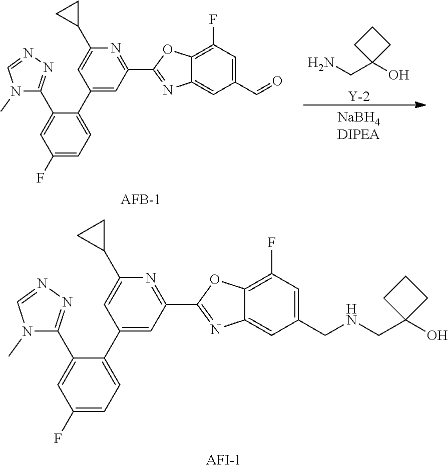

To a stirred solution of intermediate (AFB-1) (30 mg, 1 Eq, 66 μmol) and 1-(aminomethyl)cyclobutan-1-ol (Y-2) (10 mg, 1.5 Eq, 0.10 mmol) in MeOH (6 mL) was added DIPEA (34 mg, 4 Eq, 0.26 mmol) at rt. The resulting mixture was stirred for 2 h at 60° C. The mixture was allowed to cool down to rt. To the above mixture was added NaBH₄ (7 mg, 3 Eq, 0.20 mmol) dropwise at 0° C. The resulting mixture was stirred for 30 min at rt. The reaction was then quenched by the addition of 2 mL of MeOH at 0° C. The resulting mixture was concentrated in vacuo. The crude product was purified by Prep-HPLC with the following conditions (Column: XBridge Prep OBD C18 Column, 30*150 mm, 5 μm; Mobile Phase A: water (0.1% NH₄HCO₃+0.1% NH₃·H₂O), Mobile Phase B: MeCN; Flow rate: 25 mL/min; Gradient: 40% B to 52% B in 9 min; Wave Length: 254/220 nm; RT: 8.05) to afford the title compound (AFI-1) (11.3 mg, 21 μmol, 32%, 99.6% Purity) as a white solid. m/z 543.2 (M+H)⁺ (ES+). ¹H NMR (400 MHz, DMSO-d6) δ 8.50 (s, 1H), 7.88-7.82 (m, 1H), 7.81 (d, J=1.6 Hz, 1H), 7.68-7.54 (m, 3H), 7.42 (d, J=11.3 Hz, 1H), 7.11 (d, J=1.6 Hz, 1H), 4.90 (s, 1H), 3.89 (s, 2H), 3.29 (s, 3H), 2.54 (s, 2H), 2.21-2.13 (m, 1H), 2.04-1.96 (m, 2H), 1.94-1.84 (m, 2H), 1.66-1.55 (m, 1H), 1.46-1.35 (m, 1H), 1.07-1.00 (m, 2H), 0.96-1.90 (m, 2H).

### Example 415: [(2-{6-Cyclopropyl-4-[4-fluoro-2-(4-methyl-1,2,4-triazol-3-yl)phenyl]pyridin-2-yl}-7-fluoro-1,3-benzoxazol-5-yl)methyl][(1-fluorocyclobutyl)methyl]amine (AFJ-1)

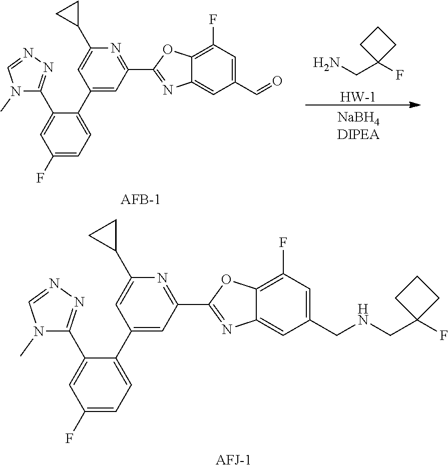

To a stirred solution of intermediate (AFB-1) (27 mg, 1 Eq, 59 μmol) and 1-(1-fluorocyclobutyl)methanamine (HW-1) (9 mg, 1.5 Eq, 88 μmol) in MeOH (6 mL) were added DIPEA (38 mg, 5 Eq, 0.30 mmol) at rt. The resulting mixture was stirred for 2 h at 60° C. The mixture was allowed to cool down to rt. To the above mixture was added NaBH₄ (7 mg, 3 Eq, 0.18 mmol) at 0° C. The resulting mixture was stirred for 2 h at rt. The reaction was then quenched by the addition of 2 mL of MeOH at 0° C. The resulting mixture was concentrated in vacuo. The crude product was purified by Prep-HPLC with the following conditions (Column: XBridge Prep OBD C18 Column, 30*150 mm, 5 μm; Mobile Phase A: water (0.1% NH₄HCO₃+0.1% NH₃·H₂O), Mobile Phase B: MeCN; Flow rate: 60 mL/min; Gradient: 47% B to 57% B in 9 min; Wave Length: 254/220 nm; RT: 8.87) to afford the sub-title compound (AFJ-1) (9.9 mg, 18 μmol, 30%, 98.0% Purity) as a white solid. m/z 545.2 (M+H)⁺ (ES+). ¹H NMR (400 MHz, DMSO-d6) δ 8.51 (s, 1H), 7.89-7.82 (m, 1H), 7.80 (d, J=1.5 Hz, 1H), 7.67-7.58 (m, 3H), 7.46-7.40 (m, 1H), 7.11 (d, J=1.6 Hz, 1H), 3.89 (s, 2H), 3.30 (s, 3H), 2.76 (s, 1H), 2.70 (s, 1H), 2.22-2.09 (m, 5H), 1.80-1.68 (m, 1H), 1.48-1.35 (m, 1H), 1.23 (s, 1H), 1.08-1.00 (m, 2H), 0.96-0.90 (m, 2H).

### Example 416: (1S,2R)-2-{[(2-{6-Cyclopropyl-4-[4-fluoro-2-(4-methyl-1,2,4-triazol-3-yl)phenyl]pyridin-2-yl}-7-fluoro-1,3-benzoxazol-5-yl)methyl](methyl)amino}cyclopentan-1-ol (AFK-1)

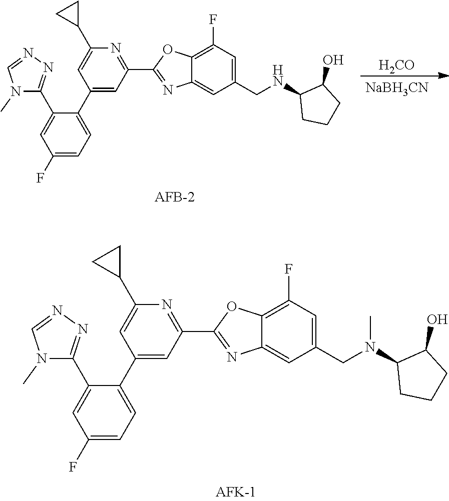 [SMILES: Cn1cnnc1-c1cc(F)ccc1-c1cc(-c2nc3cc(CN[C@@H]4CCC[C@@H]4O)cc(F)c3o2)nc(C2CC2)c1 ; "H2CO" ; "NaBH3CN" ; CN(Cc1cc(F)c2oc(-c3cc(-c4ccc(F)cc4-c4nncn4C)cc(C4CC4)n3)nc2c1)[C@@H]1CCC[C@@H]1O]

To a stirred solution of compound (AFB-2) (30 mg, 1 Eq, 55 μmol) and HCHO (8 mg, 5 Eq, 0.28 mmol) in MeOH (4 mL) was added NaBH₃CN (7 mg, 2 Eq, 0.11 mmol) at rt. The resulting mixture was stirred for 2 h at rt. The reaction was then quenched by the addition of 2 mL of MeOH at 0° C. The resulting mixture was diluted with water and extracted with DCM (3×20 mL). The combined organic layers were washed with brine (2×20 mL), dried (Na₂SO₄). After filtration, the filtrate was concentrated in vacuo. The crude product was purified by Prep-HPLC with the following conditions: Column: XBridge Prep OBD C18 Column, 30×150 mm, 5 μm; Mobile Phase A: water (0.1% NH₄HCO₃+0.1% NH₃·H₂O) Mobile Phase B: MeCN; Flow rate: 60 mL/min; Gradient: 35 B to 50 B in 8 min; Detector, UV 254/210 nm; RT: 7.9. The product-containing fractions were combined and evaporated partially in vacuo and lyophilized overnight to afford the title compound (AFK-1) (4.1 mg, 7.4 μmol, 13%, 99.5% Purity) as a white solid. m/z 557.2 (M+H)⁺ (ES+). ¹H NMR (400 MHz, Methanol-d4) δ 8.47 (s, 1H), 7.91 (d, J=1.6 Hz, 1H), 7.87-7.80 (m, 1H), 7.63-7.54 (m, 2H), 7.53-7.47 (m, 1H), 7.43-7.35 (m, 1H), 7.10 (d, J=1.6 Hz, 1H), 4.24 (t, J=3.9 Hz, 1H), 3.83 (d, J=13.3 Hz, 1H), 3.71 (d, J=13.3 Hz, 1H), 3.32-3.31 (m, 3H), 2.59 (s, 1H), 2.25-2.12 (m, 4H), 2.01-1.69 (m, 6H), 1.11-0.99 (m, 4H).

### Example 417: (1S,2R)-2-(((2-(6-Cyclopropyl-4-(4-fluoro-2-(4-methyl-4H-1,2,4-triazol-3-yl)phenyl)pyridin-2-yl)-7-fluorobenzo[d]oxazol-5-yl)methyl)amino)-1-methylcyclopentan-1-ol (AFL-2)

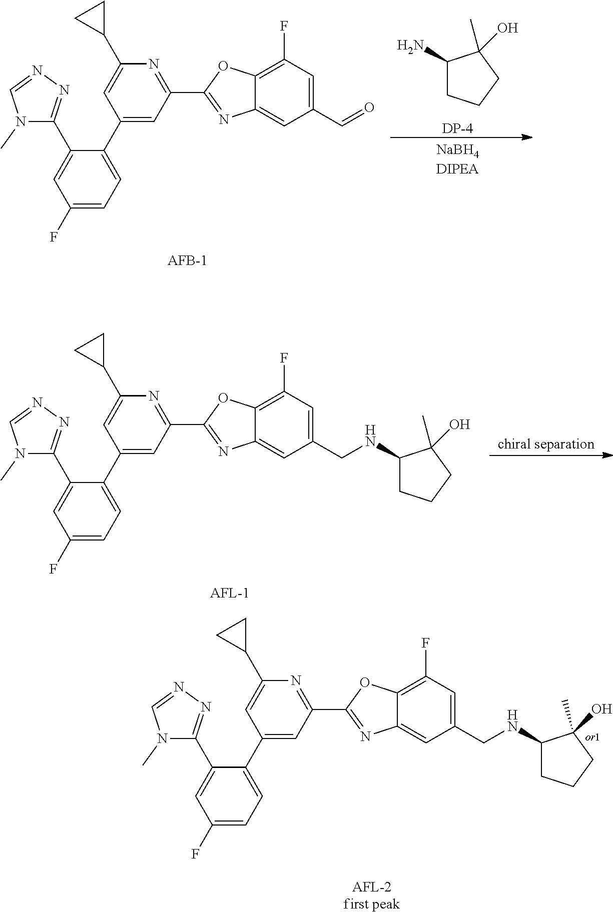

Step 1: (2R)-2-{[(2-{6-Cyclopropyl-4-[4-fluoro-2-(4-methyl-1,2,4-triazol-3-yl)phenyl]pyridin-2-yl}-7-fluoro-1,3-benzoxazol-5-yl)methyl]amino}-1-methylcyclopentan-1-ol (AFL-2). To a stirred solution of intermediate (AFB-1) (60 mg, 1 Eq, 0.13 mmol) and intermediate (DP-4) (23 mg, 1.5 Eq, 0.20 mmol) in MeOH (10 mL) were added DIPEA (26 mg, 1.5 Eq, 0.20 mmol) at rt. The resulting mixture was stirred for 2 h at 60° C. The mixture was allowed to cool down to rt. To the above mixture was added NaBH₄ (10 mg, 2 Eq, 0.26 mmol) at 0° C. The resulting mixture was stirred for additional 1 h at rt. The reaction was then quenched by the addition of 5 mL of MeOH at 0° C. and then concentrated in vacuo. The residue was purified by TLC with DCM/MeOH (10/1). The crude product was purified by Prep-HPLC with the following conditions (Column: Xselect CSH C18 OBD Column 30*150 mm, 5 μm; Mobile Phase A: water (0.1% FA), Mobile Phase B: MeCN; Flow rate: 60 mL/min; Gradient: 4% B to 34% B in 10 min; Wave Length: 254 nm) to afford the sub-title compound (AFL-1) (20 mg, 36 μmol, 27%, 99% Purity) as a white solid. m/z 557.2 (M+H)⁺ (ES+).

Step 2: (1S,2R)-2-(((2-(6-Cyclopropyl-4-(4-fluoro-2-(4-methyl-4H-1,2,4-triazol-3-yl)phenyl)pyridin-2-yl)-7-fluorobenzo[d]oxazol-5-yl)methyl)amino)-1-methylcyclopentan-1-ol (AFL-2). The crude product of the product from step 1 above (AFL-1) (20 mg, 1 Eq, 36 μmol) was purified by Prep-Chrial-HPLC with the following conditions (Column: CHIRAL ART Cellulose-SA, 2*25 cm, 5 μm; Mobile Phase A: Hex:DCM=3:1 (0.5% 2M NH₃-MeOH)-HPLC, Mobile Phase B: EtOH-HPLC; Flow rate: 20 mL/min; Gradient: 20% B to 20% B in 8 min; Wave Length: 220/254 nm; RT1(min): 5.64) to afford the title compound (AFL-2) (11.1 mg, 20 μmol, 55%, 98.7% Purity) as a white solid. m/z 557.2 (M+H)⁺ (ES+). ¹H NMR (400 MHz, DMSO-d6) δ 8.50 (s, 1H), 7.90-7.80 (m, 2H), 7.68-7.58 (m, 3H), 7.42 (d, J=11.4 Hz, 1H), 7.12 (d, J=1.6 Hz, 1H), 4.29 (s, 1H), 3.96-3.79 (m, 2H), 3.30 (s, 3H), 2.69 (t, J=7.4 Hz, 1H), 2.22-2.14 (m, 1H), 1.93-1.82 (m, 1H), 1.59-1.46 (m, 4H), 1.37-1.24 (m, 1H), 1.15 (s, 3H), 1.11-1.02 (m, 2H), 0.99-0.90 (m, 2H). Column: CHIRALPAK IA-3, 4.6*50 mm, 3 μm; Mobile Phase A: Hex:DCM=3:1) (0.1% DEA):EtOH=80:20; Flow rate: 1 mL/min; RT: 1.475.

### Example 418: (1R,2R)-2-(((2-(6-Cyclopropyl-4-(4-fluoro-2-(4-methyl-4H-1,2,4-triazol-3-yl)phenyl)pyridin-2-yl)-7-fluorobenzo[d]oxazol-5-yl)methyl)amino)-1-methylcyclopentan-1-ol (AFM-1)

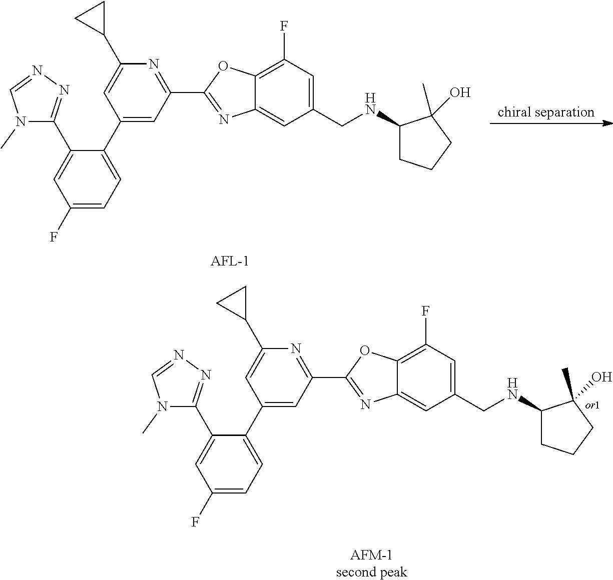

The crude product (AFL-1) (20 mg, 1 Eq, 36 μmol) was purified by Prep-Chrial-HPLC with the following conditions (Column: CHIRAL ART Cellulose-SA, 2*25 cm, 5 μm; Mobile Phase A: Hex:DCM=3:1(0.5% 2M NH₃-MeOH)-HPLC, Mobile Phase B: EtOH-HPLC; Flow rate: 20 mL/min; Gradient: 20% B to 20% B in 8 min; Wave Length: 220/254 nm; RT2(min): 7.15) to afford the title compound (AFM-1) (2.7 mg, 4.9 μmol, 13%, 99.6% Purity) as a white solid. m/z 557.2 (M+H)⁺ (ES+). ¹H NMR (400 MHz, DMSO-d6) δ 8.50 (s, 1H), 7.88-7.80 (m, 2H), 7.74-7.59 (m, 3H), 7.45 (s, 1H), 7.13 (s, 1H), 4.30 (s, 1H), 3.89 (s, 2H), 3.30 (s, 3H), 2.72 (s, 1H), 2.22-2.12 (m, 1H), 1.90 (s, 1H), 1.56 (s, 4H), 1.32 (s, 1H), 1.16 (s, 3H), 1.10-1.01 (m, 2H), 0.98-0.91 (m, 2H). Column: CHIRALPAK IA-3, 4.6*50 mm, 3 μm; Mobile Phase A: Hex:DCM=3:1) (0.1% DEA):EtOH=80:20; Flow rate: 1 mL/min; RT: 1.953.

### Example 419: [(2-{6-Cyclopropyl-4-[4-fluoro-2-(4-methyl-1,2,4-triazol-3-yl)phenyl]pyridin-2-yl}-7-fluoro-1,3-benzoxazol-5-yl)methyl][(2R)-2-methoxypropyl]amine (AFN-2)

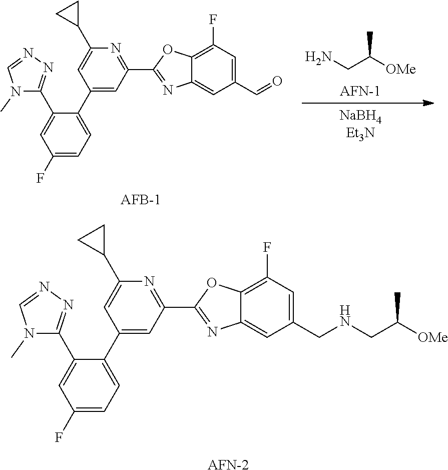

To a stirred mixture of intermediate (AFB-1) (20 mg, 1 Eq, 44 μmol) and (2R)-2-methoxypropan-1-amine (AFN-1) (8 mg, 2 Eq, 88 μmol) in MeOH (2 mL) was added Et₃N (13 mg, 3 Eq, 0.13 mmol) at rt. The resulting mixture was stirred for 1 h at 60° C. The mixture was allowed to cool down to rt. To the above mixture was added NaBH₄ (8 mg, 0.22 mmol, 5 Eq) at rt. The resulting mixture was stirred for additional 1 h at rt. The reaction was then quenched by the addition of 2 mL of MeOH at 0° C. The resulting mixture was concentrated in vacuo. The residue was purified by Prep-TLC with DCM/MeOH (10/1). The crude product was purified by Prep-HPLC with the following conditions (Column: XBridge Prep OBD C18 Column, 30*150 mm, 5 μm; Mobile Phase A: water (0.1% NH₄HCO₃+0.1% NH₃·H₂O), Mobile Phase B: MeCN; Flow rate: 60 mL/min; Gradient: 40% B to 60% B in 8 min; Wave Length: 254/220 nm; RT: 7.77) to afford the title compound (AFN-2) (4.1 mg, 7.7 μmol, 18%, 99.8% Purity) as a white solid. m/z 531.2 (M+H)⁺ (ES+). ¹H NMR (300 MHz, DMSO-d6) δ 8.50 (s, 1H), 7.90-7.79 (m, 2H), 7.68-7.59 (m, 3H), 7.44-7.36 (m, 1H), 7.11 (d, J=1.6 Hz, 1H), 3.83 (s, 2H), 3.39 (s, 1H), 3.30 (s, 3H), 3.23 (s, 3H), 2.48-2.42 (m, 2H), 2.20-2.15 (m, 1H), 1.11-1.01 (m, 5H), 0.98-0.91 (m, 2H).

### Example 420: [(2-{6-Cyclopropyl-4-[4-fluoro-2-(4-methyl-1,2,4-triazol-3-yl)phenyl]pyridin-2-yl}-7-fluoro-1,3-benzoxazol-5-yl)methyl][(2S)-2-methoxypropyl]amine (AFO-2)

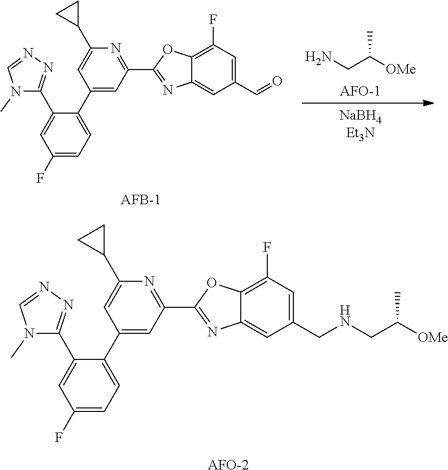

To a stirred mixture of intermediate (AFB-1) (20 mg, 1 Eq, 44 μmol) and (2S)-2-methoxypropan-1-amine (AFO-1) (8 mg, 2 Eq, 88 μmol) in MeOH (3 mL) was added Et₃N (13 mg, 3 Eq, 0.13 mmol) at rt. The resulting mixture was stirred for 2 h at 60° C. The mixture was allowed to cool down to rt. To the above mixture was added NaBH₄ (8.27 mg, 5 Eq, 0.22 mmol) at rt. The resulting mixture was stirred for additional 1 h at rt. The reaction was then quenched by the addition of 2 mL of MeOH at 0° C. The resulting mixture was concentrated in vacuo. The residue was purified by Prep-TLC with DCM/MeOH (10/1). The crude product was purified by Prep-HPLC with the following conditions (Column: XBridge Prep OBD C18 Column, 30*150 mm, 5 μm; Mobile Phase A: water (0.1% NH₄HCO₃+0.1% NH₃·H₂O), Mobile Phase B: MeCN; Flow rate: 60 mL/min; Gradient: 40% B to 60% B in 8 min; Wave Length: 254/220 nm; RT: 7.72) to afford the title compound (AFO-2) (2.0 mg, 3.8 μmol, 8.6%, 98.7% Purity) as a white solid. m/z 531.2 (M+H)⁺ (ES+). ¹H NMR (300 MHz, DMSO-d6) δ 8.50 (s, 1H), 7.90-7.79 (m, 2H), 7.68-7.59 (m, 3H), 7.44-7.36 (m, 1H), 7.11 (d, J=1.6 Hz, 1H), 3.83 (s, 2H), 3.39 (s, 1H), 3.30 (s, 3H), 3.23 (s, 3H), 2.48-2.42 (m, 2H), 2.20-2.15 (m, 1H), 1.11-1.01 (m, 5H), 0.98-0.91 (m, 2H).

### Example 421: 2-{6-Cyclopropyl-4-[4-fluoro-2-(4-methyl-1,2,4-triazol-3-yl)phenyl]pyridin-2-yl}-7-fluoro-5-[(3-fluoroazetidin-1-yl)methyl]-1,3-benzoxazole (AFP-3)

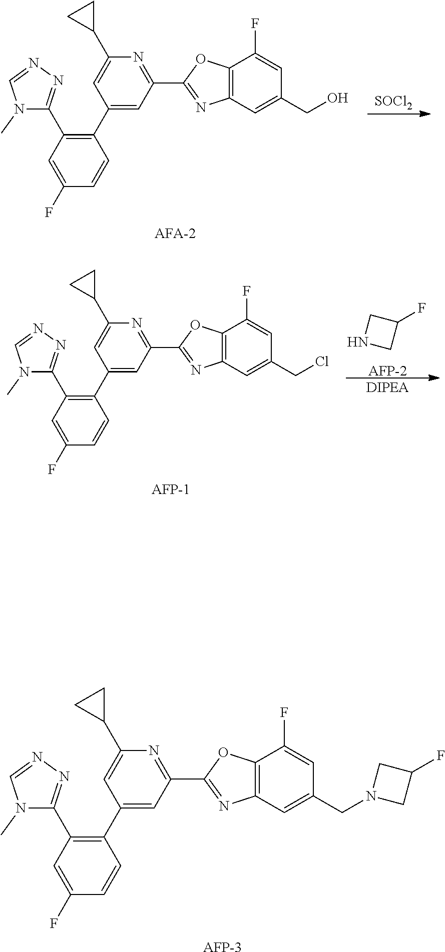

Step 1: 5-(Chloromethyl)-2-{6-cyclopropyl-4-[4-fluoro-2-(4-methyl-1,2,4-triazol-3-yl)phenyl]pyridin-2-yl}-7-fluoro-1,3-benzoxazole (AFP-1). To a stirred solution of intermediate (AFA-2) (30 mg, 1 Eq, 65 μmol) in DCM (5 mL) was added SOCl₂ (12 mg, 1.5 Eq, 98 μmol) at 0° C. The resulting mixture was stirred for 1 h at rt. The reaction was then quenched by the addition of 2 mL of MeOH at 0° C. The resulting mixture was concentrated in vacuo. The residue was purified by Prep-TLC with DCM/MeOH (10/1) to afford the sub-title compound (AFP-1) (28 mg, 59 μmol, 90%, 92% Purity) as a yellow solid. m/z 478.1/480.1 (M+H)⁺ (ES+).

Step 2: 2-{6-Cyclopropyl-4-[4-fluoro-2-(4-methyl-1,2,4-triazol-3-yl)phenyl]pyridin-2-yl}-7-fluoro-5-[(3-fluoroazetidin-1-yl)methyl]-1,3-benzoxazole (AFP-3). To a stirred solution of the product from step 1 above (AFP-1) (50 mg, 1 Eq, 0.10 mmol) and 3-fluoroazetidine (AFP-2) (13 mg, 1.7 Eq, 0.17 mmol) in DCM (8 mL) was added DIPEA (52 mg, 4 Eq, 0.40 mmol) at rt. The resulting mixture was stirred for 2 days at 60° C. under nitrogen atmosphere. The mixture was allowed to cool down to rt. The resulting mixture was concentrated in vacuo. The crude product was purified by Prep-HPLC with the following conditions (Column: Xselect CSH C18 OBD Column 30*150 mm 5 μm; Mobile Phase A: water (0.1% FA), Mobile Phase B: MeCN; Flow rate: 60 mL/min; Gradient: 13% B to 43% B in 10 min; Wave Length: 254 nm; RT: 6.58) to afford the title compound (AFP-3) (16.8 mg, 33 μmol, 32%, 99.7% Purity) as a white solid. m/z 517.6 (M+H)⁺ (ES+). ¹H NMR (400 MHz, DMSO-d6) δ 8.50 (s, 1H), 7.88-7.82 (m, 1H), 7.80 (d, J=1.5 Hz, 1H), 7.71-7.54 (m, 3H), 7.33 (d, J=11.2 Hz, 1H), 7.11 (d, J=1.5 Hz, 1H), 5.30-5.08 (m, 1H), 3.76 (s, 2H), 3.64-3.53 (m, 2H), 3.30 (s, 3H), 3.25-3.19 (m, 1H), 3.19-3.11 (m, 1H), 2.21-2.12 (m, 1H), 1.08-1.00 (m, 2H), 0.96-0.89 (m, 2H).

### Example 422: 1-({[(1S)-1-(2-{6-Cyclopropyl-4-[4-fluoro-2-(4-methyl-1,2,4-triazol-3-yl)phenyl]pyridin-2-yl}-7-fluoro-1,3-benzoxazol-5-yl)ethyl]amino}methyl)cyclobutan-1-ol (AFQ-6)

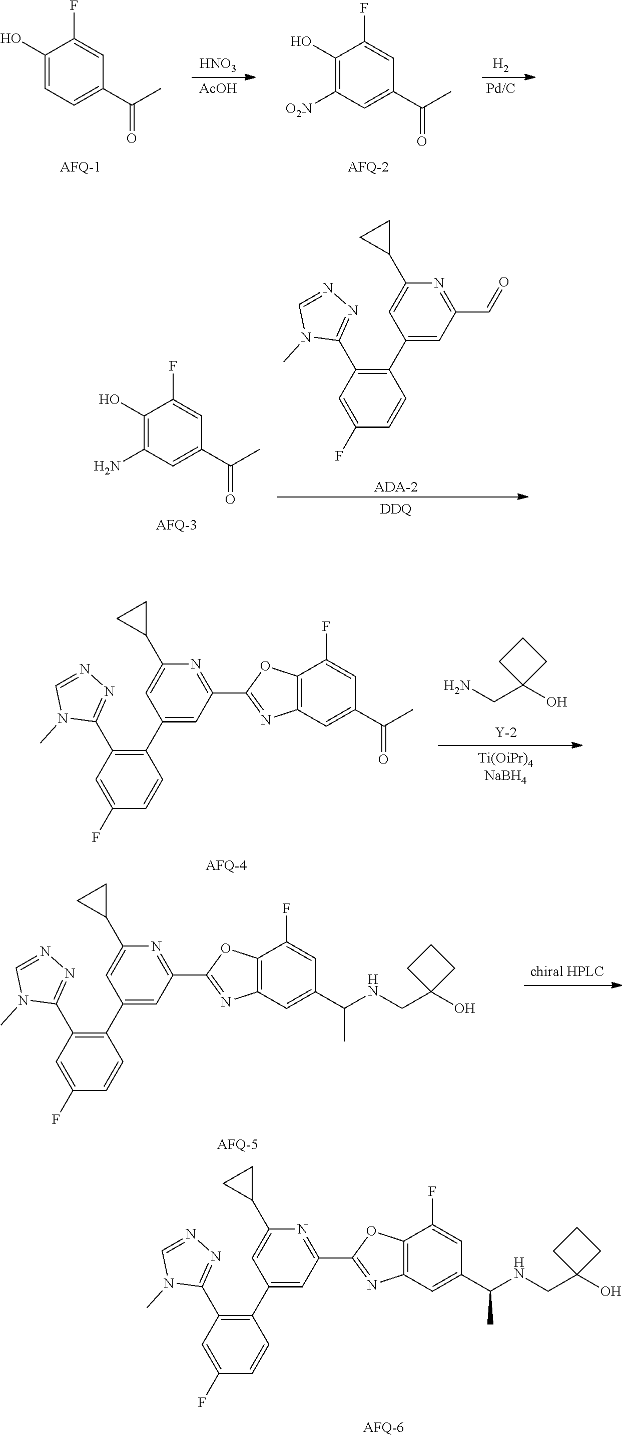

Step 1: 1-(3-Fluoro-4-hydroxy-5-nitrophenyl)ethanone (AFQ-2). A solution of 1-(3-fluoro-4-hydroxyphenyl)ethanone (AFQ-1) (2.00 g, 1 Eq, 13.0 mmol) and HNO₃ (4.91 g, 6 Eq, 77.9 mmol) in AcOH (40 mL) was stirred for 5 min at 0° C. The resulting mixture was stirred for 2 h at rt. The resulting mixture was diluted with water and extracted with EtOAc (3×100 mL). The combined organic layers were washed with brine (2×100 mL), dried (Na₂SO₄). After filtration, the filtrate was concentrated in vacuo. The residue was purified by Prep-TLC with DCM/MeOH (10/1) to afford the sub-title compound (AFQ-2) (1.8 g, 9.05 mmol, 70%, 90% Purity) as a yellow solid. m/z 200.0 (M+H)⁺ (ES+).

Step 2: 1-(3-Amino-5-fluoro-4-hydroxyphenyl)eth anone (AFQ-3). To a stirred solution of the product from step 1 above (AFQ-2) (126 mg, 1 Eq, 0.63 mmol) in MeOH (10 ml) was added Pd/C type 87L (70 mg, 10% Wt, 10 Eq, 6.33 mmol) at rt under nitrogen atmosphere. The resulting mixture was stirred for additional 1 h at rt under hydrogen atmosphere. The resulting mixture was filtered; the filter cake was washed with MeOH (3×5 mL). The filtrate was concentrated in vacuo to afford the sub-title compound (AFQ-3) (95 mg, 0.56 mmol, 84%, 91% Purity) as a brown solid. m/z 170.1 (M+H)⁺ (ES+).

Step 3: 1-(2-{6-Cyclopropyl-4-[4-fluoro-2-(4-methyl-1,2,4-triazol-3-yl)phenyl]pyridin-2-yl}-7-fluoro-1,3-benzoxazol-5-yl)ethanone (AFQ-4). To a stirred solution of intermediate (ADA-3) (120 mg, 1 Eq, 0.39 mmol) and the product from step 2 above (AFQ-3) (85 mg, 1.3 Eq, 0.50 mmol) in DCM (10 mL) at rt. The resulting mixture was stirred for 1 h at 60° C. The mixture was allowed to cool down to rt. To the above mixture was added DDQ (176 mg, 2 Eq, 0.77 mmol) at rt. The resulting mixture was stirred for 2 h at rt. The resulting mixture was diluted with water and extracted with EtOAc (3×20 mL). The combined organic layers were washed with brine (2×20 mL), dried (Na₂SO₄). After filtration, the filtrate was concentrated in vacuo. The crude product was purified by reverse flash column Chromatography with the following conditions: Column, C18; mobile phase, water (0.1% FA) and MeCN (40% MeCN up to 65% in 20 min); Detector, UV 254/220 nm. The product-containing fractions were combined and concentrated in vacuo. This resulted in the sub-title compound (AFQ-4) (100 mg, 0.21 mmol, 55%, 95% Purity) as a yellow solid. m/z 472.2 (M+H)⁺ (ES+)

Step 4: 1-(((1-(2-(6-Cyclopropyl-4-(4-fluoro-2-(4-methyl-4H-1,2,4-triazol-3-yl)phenyl)pyridin-2-yl)-7-fluorobenzo[d]oxazol-5-yl)ethyl)amino)methyl)cyclobutan-1-ol (AFQ-5). To a stirred solution of the product from step 3 above (AFQ-4) (40 mg, 1 Eq, 85 μmol) and 1-(aminomethyl)cyclobutan-1-ol (Y-2) (26 mg, 2 Eq, 0.26 mmol) in THF (8 mL) was added Ti(Oi-Pr)₄ (121 mg, 5 Eq, 0.43 mmol) for overnight at 60° C. under nitrogen atmosphere. The mixture was allowed to cool down to rt. To the above mixture was added NaBH₄ (32 mg, 10 Eq, 0.85 mmol) at 0° C. The resulting mixture was stirred for 2 h at rt. The reaction was then quenched by the addition of 2 mL of MeOH at 0° C. The mixture was concentrated in vacuo. The crude product was purified by Prep-HPLC with the following conditions: Column: XBridge Prep OBD C18 Column, 30×150 mm, 5 μm; Mobile Phase A: water (0.1% NH₄HCO₃+0.1% NH₃·H₂O) Mobile Phase B: MeCN; Flow rate: 60 mL/min; Gradient: 48 B to 58 B in 10 min; Detector, UV 254/210 nm; RT: 9.8. This resulted in the sub-title compound (AFQ-5) (15.0 mg, 27 μmol, 38%, 98% Purity) as a white solid. m/z 557.2 (M+H)⁺ (ES+)

Step 5: 1-({[(1S)-1-(2-{6-Cyclopropyl-4-[4-fluoro-2-(4-methyl-1,2,4-triazol-3-yl)phenyl]pyridin-2-yl}-7-fluoro-1,3-benzoxazol-5-yl)ethyl]amino}methyl)cyclobutan-1-ol (AFQ-6). The crude product of the product from step 4 above (AFQ-5) (15 mg, 1 Eq, 27 μmol) was purified by Prep-Chrial-HPLC with the following conditions (Column: CHIRALPAK IH, 2*25 cm, 5 μm; Mobile Phase A: MtBE (0.5% 2M NH₃-MeOH)-HPLC, Mobile Phase B: IPA-HPLC; Flow rate: 20 mL/min; Gradient: 20% B to 20% B in 36 min; Wave Length: 220/254 nm; RT1(min): 21.31) to afford the title compound (AFQ-6) (6.4 mg, 12 μmol, 13%, 98.8% Purity) as a white solid. m/z 557.2 (M+H)⁺ (ES+). ¹H NMR (400 MHz, Methanol-d4) δ 8.46 (s, 1H), 7.92 (d, J=1.5 Hz, 1H), 7.87-7.81 (m, 1H), 7.63-7.54 (m, 2H), 7.53-7.48 (m, 1H), 7.39-7.33 (m, 1H), 7.10 (d, J=1.6 Hz, 1H), 3.96 (d, J=7.1 Hz, 1H), 3.31 (s, 3H), 2.65-2.47 (m, 2H), 2.21-2.11 (m, 1H), 2.11-1.96 (m, 4H), 1.72 (d, J=16.5 Hz, 1H), 1.49-1.34 (m, 4H), 1.12-0.98 (m, 4H). Column: CHIRALPAK IH-3, 4.6*50 mm, 3 μm; Mobile Phase A: MtBE(0.1% DEA): IPA=80:20; Flow rate: 1 mL/min; RT: 2.042.

### Example 423: 1-({[(1R)-1-(2-{6-Cyclopropyl-4-[4-fluoro-2-(4-methyl-1,2,4-triazol-3-yl)phenyl]pyridin-2-yl}-7-fluoro-1,3-benzoxazol-5-yl)ethyl]amino}methyl)cyclobutan-1-ol (AFR-1)

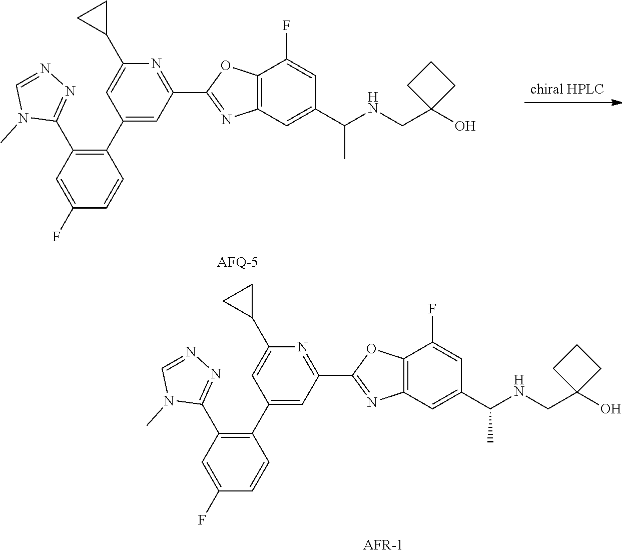

The crude product (AFQ-5) (15 mg, 1 Eq, 27 μmol) was purified by Prep-Chrial-HPLC with the following conditions (Column: CHIRALPAK IH, 2*25 cm, 5 μm; Mobile Phase A: MtBE (0.5% 2M NH₃-MeOH)-HPLC, Mobile Phase B: IPA-HPLC; Flow rate: 20 mL/min; Gradient: 20% B to 20% B in 36 min; Wave Length: 220/254 nm; RT2(min): 33.20) to afford the title compound (AFR-1) (6.3 mg, 12 μmol, 13%, 98.4% Purity) as a white solid. m/z 557.2 (M+H)⁺ (ES+). ¹H NMR (400 MHz, Methanol-d4) δ 8.46 (s, 1H), 7.92 (d, J=1.6 Hz, 1H), 7.86-7.81 (m, 1H), 7.64-7.55 (m, 2H), 7.53-7.48 (m, 1H), 7.39-7.33 (m, 1H), 7.11 (d, J=1.6 Hz, 1H), 3.98 (d, J=6.8 Hz, 1H), 3.31 (s, 3H), 2.65 (d, J=11.9 Hz, 1H), 2.50 (d, J=11.9 Hz, 1H), 2.20-2.12 (m, 1H), 2.10-1.93 (m, 4H), 1.74-1.66 (m, J=9.2, 5.0 Hz, 1H), 1.54-1.35 (m, 4H), 1.11-1.01 (m, 4H). Column: CHIRALPAK IH-3, 4.6×50 mm, 3 μm; Mobile Phase A: MtBE (0.1% DEA): IPA=80:20; Flow rate: 1 mL/min; RT: 3.265.

### Example 424: [(1S)-1-(2-{6-Cyclopropyl-4-[4-fluoro-2-(4-methyl-1,2,4-triazol-3-yl)phenyl]pyridin-2-yl}-7-fluoro-1,3-benzoxazol-5-yl)ethyl][(1-methoxycyclobutyl)methyl]amine (AFS-2)

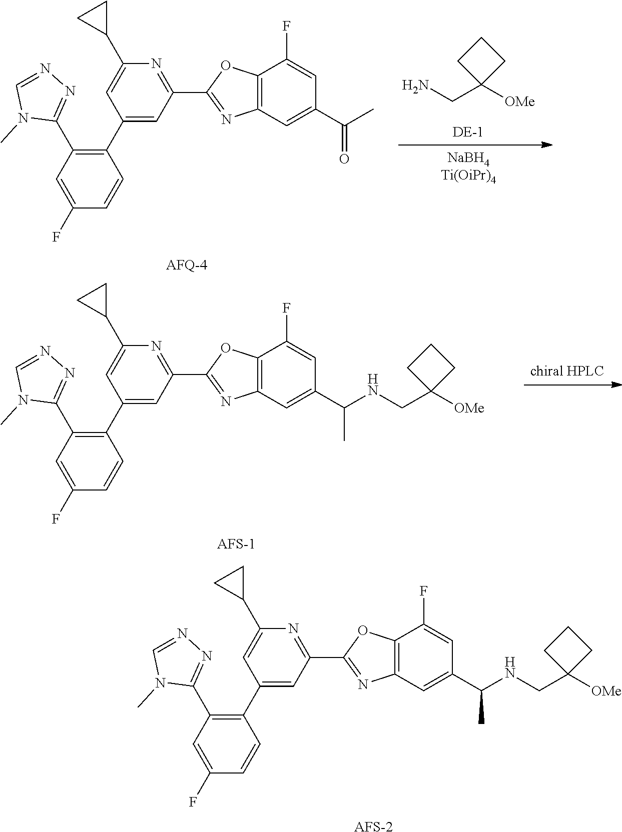

Step 1: 1-(2-(6-Cyclopropyl-4-(4-fluoro-2-(4-methyl-4H-1,2,4-triazol-3-yl)phenyl)pyridin-2-yl)-7-fluorobenzo[d]oxazol-5-yl)-N-((1-methoxycyclobutyl)methyl)ethan-1-amine (AFS-1). To a stirred mixture of intermediate (AFQ-4) (85 mg, 1 Eq, 0.18 mmol) and 1-(1-methoxycyclobutyl)methanamine (DE-1) (42 mg, 2 Eq, 0.36 mmol) in THF (6 mL) was added Ti(Oi-Pr)₄ (154 mg, 3 Eq, 0.54 mmol) at rt. The resulting mixture was stirred for 1 h at 60° C. The mixture was allowed to cool down to rt. To the above mixture was added NaBH₄ (34 mg, 5 Eq, 0.90 mmol) at rt. The resulting mixture was stirred for additional 1 h at rt. The reaction was then quenched by the addition of 2 mL of MeOH at 0° C. he resulting mixture was concentrated in vacuo. The residue was purified by Prep-TLC with DCM/MeOH (10/1). The crude product was purified by Prep-HPLC with the following conditions: Column: XBridge Prep OBD C18 Column, 30*150 mm, 5 μm; Mobile Phase A: water (0.1% NH₄HCO₃+0.1% NH₃·H₂O), Mobile Phase B: MeCN; Flow rate: 60 mL/min; Gradient: 55% B to 65% B in 8 min; Wave Length: 254/220 nm; RT: 6.94) to afford the sub-title compound (AFS-1) (20 mg, 35 μmol, 19%, 98% Purity) as a white solid. m/z 571.2 (M+H)⁺ (ES+).

Step 2: [(1S)-1-(2-{6-Cyclopropyl-4-[4-fluoro-2-(4-methyl-1,2,4-triazol-3-yl)phenyl]pyridin-2-yl}-7-fluoro-1,3-benzoxazol-5-yl)ethyl][(1-methoxycyclobutyl)methyl]amine (AFS-2). The crude product of the product from step 1 above (AFS-1) (20 mg, 1 Eq, 35 μmol) was purified by Prep-Chrial-HPLC with the following conditions (Column: CHIRALPAK ID, 2*25 cm, 5 μm; Mobile Phase A: Hex:DCM=3:1(0.5% 2M NH₃-MeOH)-HPLC, Mobile Phase B: EtOH-HPLC; Flow rate: 20 mL/min; Gradient: 20% B to 20% B in 22 min; Wave Length: 220/254 nm; RT1(min): 12.579) to afford the title compound (AFS-2) (7.7 mg, 14 μmol, 39%, 99.8% Purity) as a white solid. m/z 571.2 (M+H)⁺ (ES+). ¹H NMR (300 MHz, DMSO-d6) δ 8.50 (s, 1H), 7.89-7.82 (m, 1H), 7.80 (d, J=1.6 Hz, 1H), 7.69-7.58 (m, 3H), 7.45 (d, J=11.5 Hz, 1H), 7.12 (d, J=1.5 Hz, 1H), 3.88 (d, J=7.0 Hz, 1H), 3.29 (s, 3H), 3.00 (s, 3H), 2.51 (d, J=1.8 Hz, 1H), 2.39 (d, J=12.2 Hz, 1H), 2.19 (d, J=8.0 Hz, 1H), 1.97 (d, J=10.2 Hz, 2H), 1.83 (s, 2H), 1.62 (d, J=10.7 Hz, 1H), 1.46-1.38 (m, 1H), 1.33 (d, J=6.5 Hz, 3H), 1.04 (d, J=7.7 Hz, 2H), 0.93 (d, J=4.8 Hz, 2H). Column: CHIRALPAK ID-3, 4.6*50 mm 3 um; Mobile Phase A: Hex:DCM=3:1) (0.1% DEA): IPA=80:20; Flow rate: 1 mL/min; RT: 2.146.

### Example 425: [(1R)-1-(2-{6-Cyclopropyl-4-[4-fluoro-2-(4-methyl-1,2,4-triazol-3-yl)phenyl]pyridin-2-yl}-7-fluoro-1,3-benzoxazol-5-yl)ethyl][(1-methoxycyclobutyl)methyl]amine (AFT-1)

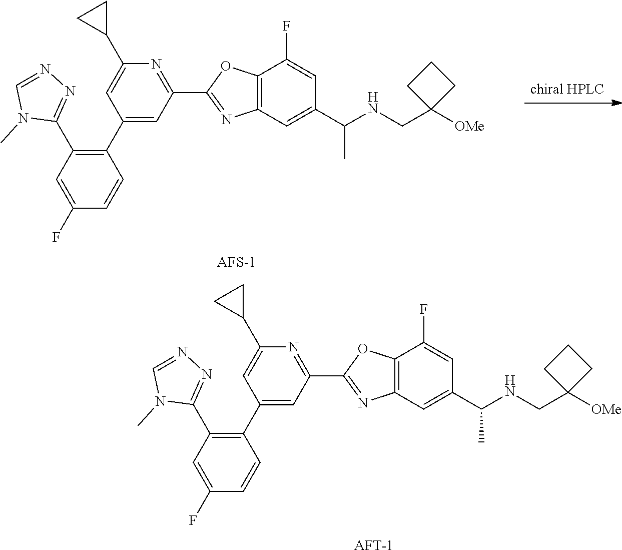 [SMILES: COC1(CNC(C)c2cc(F)c3oc(-c4cc(-c5ccc(F)cc5-c5nncn5C)cc(C5CC5)n4)nc3c2)CCC1 ; "AFS-1" ; COC1(CN[C@H](C)c2cc(F)c3oc(-c4cc(-c5ccc(F)cc5-c5nncn5C)cc(C5CC5)n4)nc3c2)CCC1 ; "AFT-1"]

The crude product (AFS-1) (20 mg, 1 Eq, 35 μmol) was purified by Prep-Chrial-HPLC with the following conditions (Column: CHIRALPAK ID, 2*25 cm, 5 μm; Mobile Phase A: Hex:DCM=3:1 (0.5% 2M NH₃-MeOH)-HPLC, Mobile Phase B: EtOH-HPLC; Flow rate: 20 mL/min; Gradient: 20% B to 20% B in 22 min; Wave Length: 220/254 nm; RT2(min): 16.647) to afford the title compound (AFT-1) (6.0 mg, 11 μmol, 30%, 99.7% Purity) as a white solid. m/z 571.2 (M+H)⁺ (ES+). ¹H NMR (300 MHz, DMSO-d6) δ 8.50 (s, 1H), 7.89-7.78 (m, 2H), 7.70-7.59 (m, 3H), 7.45 (d, J=11.5 Hz, 1H), 7.11 (s, 1H), 3.88 (d, J=7.0 Hz, 1H), 3.29 (s, 3H), 3.00 (s, 3H), 2.51 (d, J=1.8 Hz, 1H), 2.37 (s, 1H), 2.17 (s, 1H), 1.97 (d, J=10.2 Hz, 2H), 1.83 (s, 2H), 1.64 (s, 1H), 1.43 (d, J=9.4 Hz, 1H), 1.33 (d, J=6.5 Hz, 3H), 1.04 (d, J=8.1 Hz, 2H), 0.93 (s, 2H). Column: CHIRALPAK ID-3, 4.6*50 mm, 3 μm; Mobile Phase A: Hex:DCM=3:1) (0.1% DEA): IPA=80:20; Flow rate: 1 mL/min; RT: 2.771.

### Example 426: [(2-{6-Cyclopropyl-4-[4-fluoro-2-(4-methyl-1,2,4-triazol-3-yl) phenyl]pyridin-2-yl}-7-fluoro-1, 3-benzoxazol-5-yl) methyl][(2S)-1-methoxypropan-2-yl]amine (AFU-1)

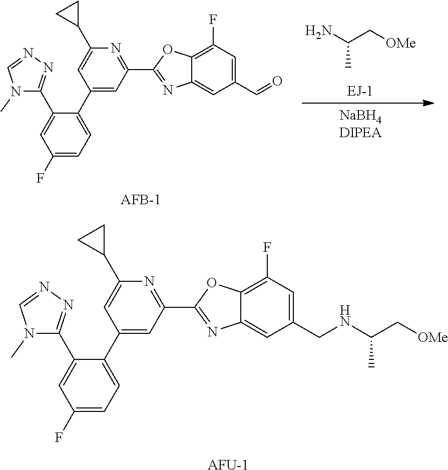

To a stirred solution of intermediate (AFB-1) (30 mg, 1 Eq, 66 μmol) and (2S)-1-methoxypropan-2-amine (EJ-1) (7 mg, 1.2 Eq, 79 μmol) in MeOH (5 mL) was added DIPEA (25 mg, 3 Eq, 0.20 mmol) at rt. The resulting mixture was stirred for overnight at 60° C. The mixture was allowed to cool down to rt. To the above mixture was added NaBH₄ (12 mg, 5 Eq, 0.33 mmol) at 0° C. The resulting mixture was stirred for additional 2 h at rt. The reaction was then quenched by the addition of 2 mL of MeOH at 0° C. The resulting mixture was concentrated in vacuo. The crude product was purified by Prep-HPLC with the following conditions (Column: XBridge Prep OBD C18 Column, 30*150 mm, 5 μm; Mobile Phase A: water (0.1% NH₄HCO₃+0.1% NH₃·H₂O), Mobile Phase B: MeCN; Flow rate: 60 mL/min; Gradient: 37% B to 47% B in 9 min; Wave Length: 254/220 nm) to afford the title compound (AFU-1) (10.8 mg, 0.02 mmol, 31%, 99.0% Purity) as a white solid. m/z 531.1 (M+H)⁺ (ES+). ¹H NMR (400 MHz, Methanol-d4) δ 8.46 (s, 1H), 7.92 (d, J=1.6 Hz, 1H), 7.87-7.80 (m, 1H), 7.65-7.54 (m, 2H), 7.53-7.46 (m, 1H), 7.37-7.30 (m, 1H), 7.10 (d, J=1.6 Hz, 1H), 3.99 (d, J=13.6 Hz, 1H), 3.88 (d, J=13.5 Hz, 1H), 3.34 (s, 4H), 3.33-3.31 (m, 4H), 2.96-2.86 (m, 1H), 2.24-2.13 (m, 1H), 1.14-1.01 (m, 7H).

### Example 427: [(2-{6-Cyclopropyl-4-[4-fluoro-2-(4-methyl-1,2,4-triazol-3-yl) phenyl]pyridin-2-yl}-7-fluoro-1, 3-benzoxazol-5-yl) methyl][(2R)-1-methoxypropan-2-yl]amine (AFV-1)

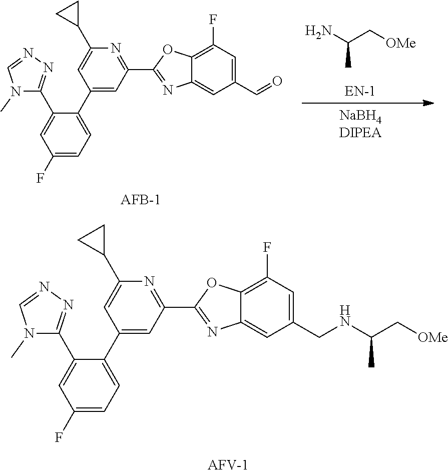

To a stirred solution of intermediate (AFB-1) (30 mg, 1 Eq, 66 μmol) and (2R)-1-methoxypropan-2-amine (EN-1) (7 mg, 1.2 Eq, 79 μmol) in MeOH (5 mL) was added DIPEA (25 mg, 3 Eq, 0.20 mmol) at rt. The resulting mixture was stirred for overnight at 60° C. The mixture was allowed to cool down to rt. To the above mixture was added NaBH₄ (12 mg, 5 Eq, 0.33 mmol) at 0° C. The resulting mixture was stirred for additional 2 h at rt. The reaction was then quenched by the addition of 2 mL of MeOH at 0° C. The resulting mixture was concentrated in vacuo. The crude product was purified by Prep-HPLC with the following conditions (Column: XBridge Prep OBD C18 Column, 30*150 mm, 5 μm; Mobile Phase A: water (0.1% NH₄HCO₃+0.1% NH₃·H₂O), Mobile Phase B: MeCN; Flow rate: 60 mL/min; Gradient: 37% B to 47% B in 9 min; Wave Length: 254/220 nm) to afford the title compound (AFV-1) (10.3 mg, 19 μmol, 29%, 98.7% Purity) as a white solid. m/z 531.1 (M+H)⁺ (ES+). ¹H NMR (400 MHz, Methanol-d4) δ 8.46 (s, 1H), 7.92 (d, J=1.6 Hz, 1H), 7.87-7.79 (m, 1H), 7.64-7.54 (m, 2H), 7.53-7.47 (m, 1H), 7.36-7.28 (m, 1H), 7.10 (d, J=1.6 Hz, 1H), 4.03-3.85 (m, 2H), 7.39-7.33 (m, 5H), 7.33-7.31 (m, 3H), 2.98-2.85 (m, 1H), 2.20-2.10 (m, 1H), 1.13-1.01 (m, 7H).

### Example 428: Methyl 2-{6-cyclopropyl-4-[4-fluoro-2-(4-methyl-1,2,4-triazol-3-yl)phenyl]pyridin-2-yl}-6,7-difluoro-1,3-benzoxazole-5-carboxylate (AFW-1)

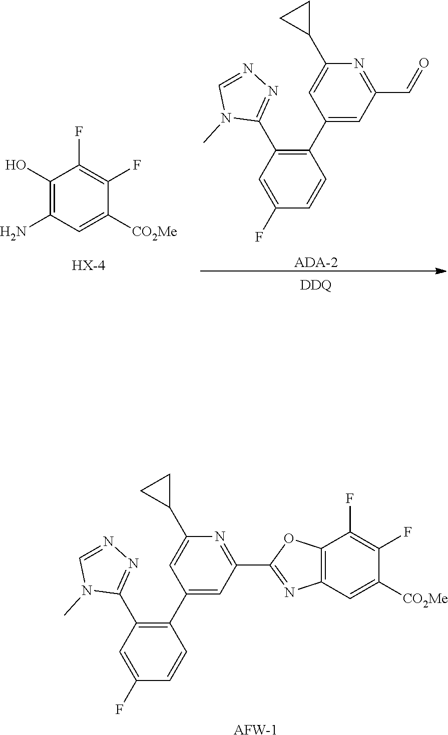

To a stirred mixture of intermediate (ADA-2) (30 mg, 1 Eq, 93 μmol) and intermediate (HX-4) (19 mg, 1 Eq, 93 μmol) in DCM (4 mL) at rt. The resulting mixture was stirred for 1 h at 60° C. The mixture was allowed to cool down to rt. To the above mixture was added DDQ (42 mg, 2 Eq, 0.19 mmol) at rt. The resulting mixture was stirred for 2 h at rt. The resulting mixture was diluted with water and extracted with DCM (3×10 mL). The combined organic layers were washed with brine (2×10 mL), dried (Na₂SO₄). After filtration, the filtrate was concentrated in vacuo. The residue was purified by Prep-TLC with DCM/MeOH (10/1). The crude product was purified by Prep-HPLC with the following conditions (Column: XBridge Prep OBD C18 Column, 30*150 mm, 5 μm; Mobile Phase A: water (0.1% NH₄HCO₃+0.1% NH₃·H₂O), Mobile Phase B: ACN; Flow rate: 60 mL/min; Gradient: 50% B to 65% B in 8 min; Wave Length: 254/220 nm; RT: 7.52) to afford the title compound (AFW-1) (6.9 mg, 14 μmol, 15%, 99.4% Purity) as a white solid. m/z 506.0 (M+H)⁺ (ES+). ¹H NMR (300 MHz, DMSO-d6) δ 8.51 (s, 1H), 8.17 (d, J=5.1 Hz, 1H), 7.93-7.79 (m, 2H), 7.64 (t, J=8.2 Hz, 2H), 7.16 (d, J=1.6 Hz, 1H), 3.93 (s, 3H), 3.32 (s, 3H), 2.17 (d, J=4.8 Hz, 1H), 1.04 (d, J=7.8 Hz, 2H), 0.94 (s, 2H).

### Example 429: (2-{6-Cyclopropyl-4-[4-fluoro-2-(4-methyl-1,2,4-triazol-3-yl)phenyl]pyridin-2-yl}-6,7-difluoro-1,3-benzoxazol-5-yl)methanol (AFX-1)

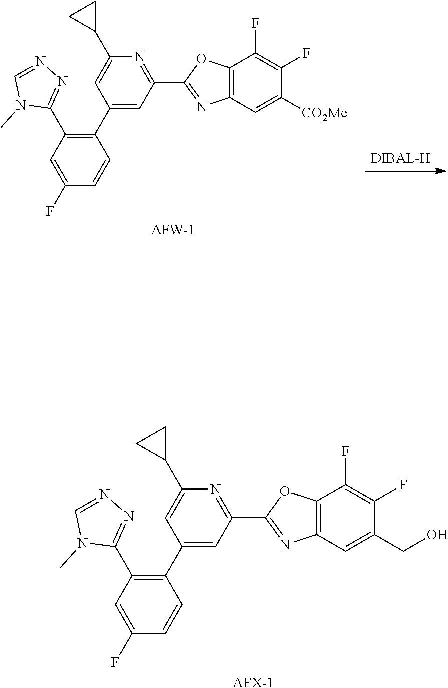 [SMILES: COC(=O)c1cc2nc(-c3cc(-c4ccc(F)cc4-c4nncn4C)cc(C4CC4)n3)oc2c(F)c1F ; "AFW-1" ; Cn1cnnc1-c1cc(F)ccc1-c1cc(-c2nc3cc(CO)c(F)c(F)c3o2)nc(C2CC2)c1 ; "AFX-1"]

To a stirred mixture of intermediate (AFW-1) (30 mg, 1 Eq, 59 μmol) in THF (4 mL) was added DIBAL-H in THF (0.18 mL, 1 M, 3 Eq, 0.18 mmol) dropwise at 0° C. under nitrogen atmosphere. The resulting mixture was stirred for 1 h at 0° C. under nitrogen atmosphere. The reaction was then quenched by the addition of 2 mL of ice water at 0° C. The resulting mixture was diluted with water and extracted with DCM (3×10 mL). The combined organic layers were washed with brine (2×10 mL), dried (Na₂SO₄). After filtration, the filtrate was concentrated in vacuo. The residue was purified by Prep-TLC with DCM/MeOH (10/1). The crude product was purified by Prep-HPLC with the following conditions (Column: XBridge Prep OBD C18 Column, 30*150 mm, 5 μm; Mobile Phase A: water (0.1% NH₄HCO₃+0.1% NH₃·H₂O), Mobile Phase B: MeCN; Flow rate: 60 mL/min; Gradient: 35% B to 45% B in 8 min; Wave Length: 254/220 nm; RT: 7.97) to afford the title compound (AFX-1) (6.2 mg, 13 μmol, 22%, 99.3% Purity) as a white solid. m/z 478.0 (M+H)⁺ (ES+). ¹H NMR (300 MHz, DMSO-d6) δ 8.51 (s, 1H), 7.90-7.78 (m, 2H), 7.72 (d, J=5.4 Hz, 1H), 7.63 (t, J=8.3 Hz, 2H), 7.12 (d, J=1.6 Hz, 1H), 5.56 (t, J=5.8 Hz, 1H), 4.68 (d, J=5.7 Hz, 2H), 3.30 (s, 3H), 2.18 (d, J=8.1 Hz, 1H), 1.04 (d, J=7.8 Hz, 2H), 1.06-0.90 (m, 2H).

### Example 430: (1S,2R)-2-{[(2-{6-Cyclopropyl-4-[4-fluoro-2-(4-methyl-1,2,4-triazol-3-yl)phenyl]pyridin-2-yl}-6,7-difluoro-1,3-benzoxazol-5-yl)methyl]amino}cyclopentan-1-ol (AFY-2)

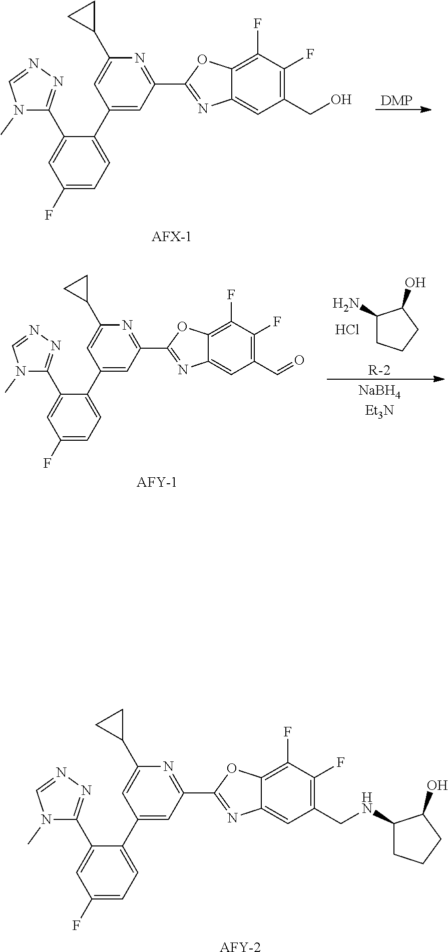

Step 1: 2-{6-Cyclopropyl-4-[4-fluoro-2-(4-methyl-1,2,4-triazol-3-yl)phenyl]pyridin-2-yl}-6,7-difluoro-1,3-benzoxazole-5-carbaldehyde (AFY-1). To a stirred mixture of intermediate (AFX-1) (100 mg, 1 Eq, 0.21 mmol) in DCM (5 mL) was added DMP (133 mg, 1.5 Eq, 0.31 mmol) at rt. The resulting mixture was stirred for 1 h at rt. The resulting mixture was concentrated in vacuo. The residue was purified by Prep-TLC with DCM/MeOH (10/1) to afford the sub-title compound (AFY-1) (70 mg, 0.15 mmol, 70%, 95% Purity) as a yellow solid. m/z 476.1 (M+H)⁺ (ES+).

Step 2: (1S,2R)-2-{[(2-{6-Cyclopropyl-4-[4-fluoro-2-(4-methyl-1,2,4-triazol-3-yl)phenyl]pyridin-2-yl}-6,7-difluoro-1,3-benzoxazol-5-yl)methyl]amino}cyclopentan-1-ol (AFY-2). To a stirred mixture of the product from step 1 above (AFY-1) (30 mg, 1 Eq, 63 μmol) and (1S,2R)-2-aminocyclopentan-1-ol (R-2) (13 mg, 2 Eq, 0.13 mmol) in MeOH (4 mL) was added Et₃N (19 mg, 3 Eq, 0.19 mmol) at rt. The resulting mixture was stirred for 2 h at 60° C. The mixture was allowed to cool down to rt. To the above mixture was added NaBH₄ (12 mg, 5 Eq, 0.32 mmol) at rt. The resulting mixture was stirred for additional 1 h at rt. The reaction was then quenched by the addition of 2 mL of MeOH at 0° C. The resulting mixture was concentrated in vacuo. The residue was purified by Prep-TLC with DCM/MeOH (10/1). The crude product was purified by Prep-HPLC with the following conditions (Column: XBridge Prep OBD C18 Column, 30*150 mm, 5 μm; Mobile Phase A: water (0.1% NH₄HCO₃+0.1% NH₃·H₂O), Mobile Phase B: MeCN; Flow rate: 60 mL/min; Gradient: 42% B to 55% B in 8 min; Wave Length: 254/220 nm; RT: 7.57) to afford the title compound (AFY-2) (8.0 mg, 14 μmol, 23%, 99.6% Purity) as a white solid. m/z 561.1 (M+H)⁺ (ES+). ¹H NMR (300 MHz, DMSO-d6) δ 8.51 (s, 1H), 7.90-7.75 (m, 3H), 7.68-7.60 (m, 2H), 7.14 (d, J=1.6 Hz, 1H), 4.43 (s, 1H), 3.93 (d, J=15.4 Hz, 3H), 3.30 (s, 3H), 2.80 (s, 1H), 2.21-2.15 (m, 1H), 1.72-1.60 (m, 4H), 1.47-1.39 (m, 2H), 1.06-0.90 (m, 4H).

### Example 431: [(2-{6-Cyclopropyl-4-[4-fluoro-2-(4-methyl-1,2,4-triazol-3-yl)phenyl]pyridin-2-yl}-7-methyl-1,3-benzoxazol-5-yl)methyl][(2R,3R)-3-methoxybutan-2-yl]amine (AFZ-2)

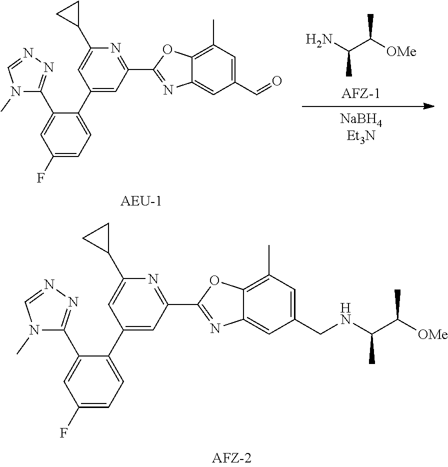

To a stirred solution of intermediate (AEU-1) (30 mg, 1 Eq, 66 μmol) and (2R,3R)-3-methoxybutan-2-amine (AFZ-1) (14 mg, 2 Eq, 0.13 mmol) in MeOH (5 mL) was added Et₃N (33. mg, 5 Eq, 0.33 mmol) for 2 h at 60° C. under nitrogen atmosphere. The mixture was allowed to cool down to rt. To the above mixture was added NaBH₄ (25 mg, 10 Eq, 0.66 mmol) at 0° C. The resulting mixture was stirred for 1 h at rt. The reaction was then quenched by the addition of 2 mL of MeOH at 0° C. The resulting mixture was diluted with water and extracted with DCM (3×10 mL). The combined organic layers were washed with brine (2×10 mL), dried (Na₂SO₄). After filtration, the filtrate was concentrated in vacuo. The crude product was purified by Prep-HPLC with the following conditions: Column: XBridge Prep OBD C18 Column, 30×150 mm, 5 μm; Mobile Phase A: water (0.1% NHLHCO₃+0.1% NH₃·H₂O) Mobile Phase B: MeCN; Flow rate: 60 mL/min; Gradient: 45 B to 55 B in 8 min; Detector, UV 254/210 nm; RT: 6.57. The product-containing fractions were combined and evaporated partially in vacuo and lyophilized overnight to afford the title compound (AFZ-2) (11.0 mg, 0.02 mmol, 31%, 99.6% Purity) as a white solid. m/z 541.2 (M+H)⁺ (ES+). ¹H NMR (400 MHz, DMSO-d6) δ 8.51 (s, 1H), 7.89-7.83 (m, 1H), 7.74 (d, J=1.5 Hz, 1H), 7.66-7.61 (m, 2H), 7.61-7.55 (m, 1H), 7.27 (s, 1H), 7.08 (d, J=1.6 Hz, 1H), 3.90-3.70 (m, 2H), 3.29 (s, 3H), 3.22 (s, 3H), 2.70-2.60 (m, 1H), 2.54 (s, 3H), 2.21-2.12 (m, 1H), 2.06 (s, 1H), 1.08-0.96 (m, 5H), 0.96-0.88 (m, 5H).

### Example 432: 1-({[(2-{6-Cyclopropyl-4-[4-fluoro-2-(4-methyl-1,2,4-triazol-3-yl)phenyl]pyridin-2-yl}-6,7-difluoro-1,3-benzoxazol-5-yl)methyl]amino}methyl)cyclobutan-1-ol (AGA-1)

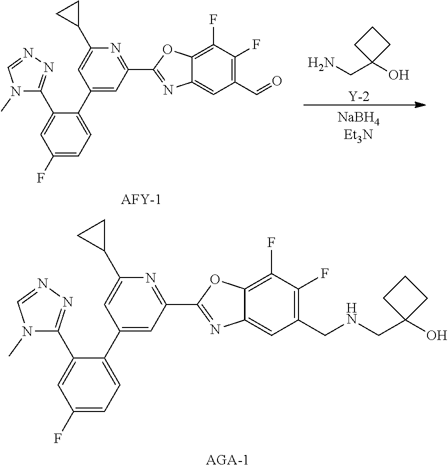

To a stirred mixture of intermediate (AFY-1) (30 mg, 1 Eq, 63 μmol) and 1-(aminomethyl)cyclobutan-1-ol (Y-2) (13 mg, 2 Eq, 0.13 mmol) in MeOH (5 mL) was added Et₃N (19 mg, 3 Eq, 0.19 mmol) at rt. The resulting mixture was stirred for 2 h at 60° C. The mixture was allowed to cool down to rt. To the above mixture was added NaBH₄ (12 mg, 5 Eq, 0.32 mmol) at rt. The resulting mixture was stirred for additional 2 h at rt. The reaction was then quenched by the addition of 2 mL of MeOH at 0° C. The resulting mixture was concentrated in vacuo. The residue was purified by Prep-TLC with DCM/MeOH (10/1). The crude product was purified by Prep-HPLC with the following conditions (Column: XBridge Prep OBD C18 Column, 30*150 mm, 5 μm; Mobile Phase A: water (0.1% NH₄HCO₃+0.1% NH₃·H₂O), Mobile Phase B: MeCN; Flow rate: 60 mL/min; Gradient: 40% B to 50% B in 8 min; Wave Length: 254/220 nm; RT: 7.6) to afford the title compound (AGA-1) (15.6 mg, 28 μmol, 44%, 96.3% Purity) as a white solid. m/z 561.1 (M+H)⁺ (ES+). ¹H NMR (300 MHz, DMSO-d6) δ 8.51 (s, 1H), 7.91-7.75 (m, 3H), 7.70-7.59 (m, 2H), 7.12 (d, J=1.6 Hz, 1H), 4.95 (s, 1H), 3.94 (s, 2H), 3.30 (s, 3H), 2.58 (s, 2H), 2.18 (m, 1H), 2.05-1.86 (m, 4H), 1.78-1.52 (m, 1H), 1.52-1.27 (m, 1H), 1.12-0.90 (m, 4H).

### Example 433: [(2-{6-Cyclopropyl-4-[4-fluoro-2-(4-methyl-1,2,4-triazol-3-yl)phenyl]pyridin-2-yl}-6,7-difluoro-1,3-benzoxazol-5-yl)methyl](2-methoxyethyl)amine (AGB-1)

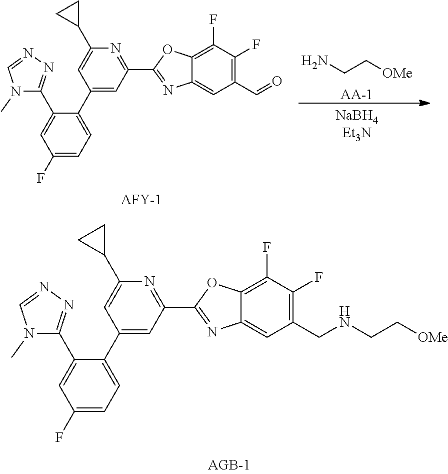

To a stirred mixture of intermediate (AFY-1) (30 mg, 1 Eq, 63 μmol) and 2-methoxyethan-1-amine (AA-1) (5 mg, 1 Eq, 63 μmol) in MeOH (5 mL) was added Et₃N (19 mg, 3 Eq, 0.19 mmol) at rt. The resulting mixture was stirred for 2 h at 60° C. The mixture was allowed to cool down to rt. To the above mixture was added NaBH₄ (12 mg, 5 Eq, 0.32 mmol) at rt. The resulting mixture was stirred for additional 2h at rt. The reaction was then quenched by the addition of 2 mL of MeOH at 0° C. The resulting mixture was concentrated in vacuo. The residue was purified by Prep-TLC with DCM/MeOH (10/1). The crude product was purified by Prep-HPLC with the following conditions (Column: XBridge Prep OBD C18 Column, 30*150 mm, 5 μm; Mobile Phase A: water (0.1% NH₄HCO₃+0.1% NH₃·H₂O), Mobile Phase B: MeCN; Flow rate: 60 mL/min; Gradient: 42% B to 52% B in 8 min; Wave Length: 254/220 nm; RT: 7.77) to afford the sub-title compound (AGB-1) (15.0 mg, 28 μmol, 44%, 99.4% Purity) as a white solid. m/z 535.0 (M+H)⁺ (ES+). ¹H NMR (300 MHz, DMSO-d6) δ 8.50 (s, 1H), 7.90-7.82 (m, 1H), 7.81-7.74 (m, 2H), 7.70-7.59 (m, 2H), 7.13 (d, J=1.6 Hz, 1H), 3.89 (s, 2H), 3.41 (t, J=5.6 Hz, 2H), 3.30 (s, 3H), 3.24 (s, 3H), 2.68 (t, J=5.6 Hz, 2H), 2.22-2.15 (m, 1H), 1.08-0.91 (m, 4H).

### Example 434: [(2-{6-Cyclopropyl-4-[4-fluoro-2-(4-methyl-1,2,4-triazol-3-yl)phenyl]pyridin-2-yl}-7-methyl-1,3-benzoxazol-5-yl)methyl][(2S,3S)-3-methoxybutan-2-yl]amine (AGC-2)

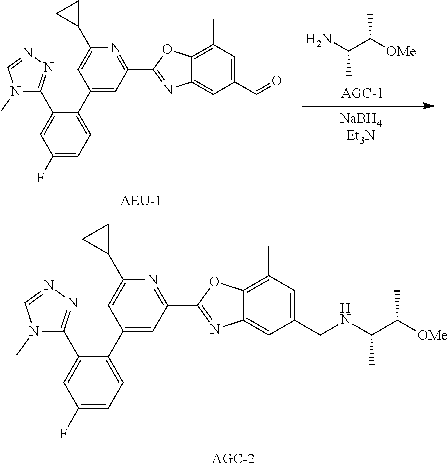

To a stirred solution of intermediate (AEU-1) (15 mg, 1 Eq, 33 μmol) and (2S,3S)-3-methoxybutan-2-amine (AGC-1) (4 mg, 1 Eq, 33 μmol) in MeOH (5 mL) was added Et₃N (17 mg, 5 Eq, PP-1005,C3 0.17 mmol) for 2 h at 60° C. under nitrogen atmosphere. The mixture was allowed to cool down to rt. To the above mixture was added NaBH₄ (13 mg, 10 Eq, 0.33 mmol) at 0° C. The resulting mixture was stirred for 2 h at rt. The reaction was then quenched by the addition of 2 mL of MeOH at 0° C. The resulting mixture was diluted with water and extracted with DCM (3×10 mL). The combined organic layers were washed with brine (2×10 mL), dried (Na₂SO₄). After filtration, the filtrate was concentrated in vacuo. The crude product was purified by Prep-HPLC with the following conditions: Column: XBridge Prep OBD C18 Column, 30×150 mm, 5 μm; Mobile Phase A: water (0.1% NH₄HCO₃+0.1% NH₃·H₂O) Mobile Phase B: MeCN; Flow rate: 60 mL/min; Gradient: 40% B to 50% B in 8 min; Detector, UV 254/210 nm; RT: 7.45. The product-containing fractions were combined and evaporated partially in vacuo and lyophilized overnight to afford the title compound (AGC-2) (7.5 mg, 14 μmol, 42%, 99.2% Purity) as a white solid. m/z 541.7 (M+H)⁺ (ES+). ¹H NMR (400 MHz, Methanol-d4) δ 8.47 (s, 1H), 7.88-7.79 (m, 2H), 7.62-7.47 (m, 3H), 7.28 (s, 1H), 7.10 (t, J=1.3 Hz, 1H), 3.98 (d, J=13.1 Hz, 1H), 3.75 (s, 1H), 3.33 (s, 3H), 3.32-3.31 (m, 3H), 3.21-3.13 (m, 1H), 2.61 (s, 4H), 2.20-2.10 (m, 1H), 1.13-1.04 (m, 10H).

### Example 435: (2-Cyclobutylpropan-2-yl)[(2-{6-cyclopropyl-4-[4-fluoro-2-(4-methyl-1,2,4-triazol-3-yl)phenyl]pyridin-2-yl}-6,7-difluoro-1,3-benzoxazol-5-yl)methyl]amine (AGD-1)

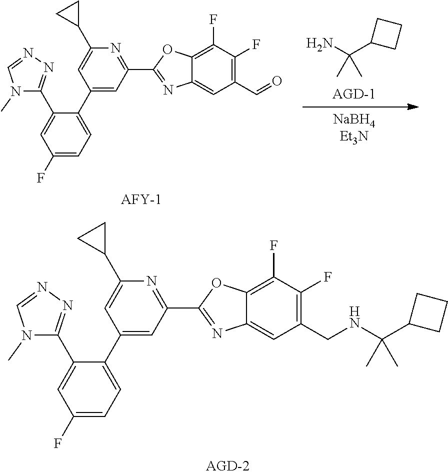

To a stirred mixture of intermediate (AFY-1) (35 mg, 1 Eq, 74 μmol) and 2-cyclobutylpropan-2-amine (AGD-1) (17 mg, 2 Eq, 0.15 mmol) in MeOH (5 mL) was added Et₃N (22 mg, 3 Eq, 0.22 mmol) at rt. The resulting mixture was stirred for 2 h at 60° C. The mixture was allowed to cool down to rt. To the above mixture was added NaBH₄ (14 mg, 5 Eq, 0.37 mmol) at rt. The resulting mixture was stirred for additional 2 h at rt. The reaction was then quenched by the addition of 2 mL of MeOH at 0° C. The resulting mixture was concentrated in vacuo. The residue was purified by Prep-TLC with DCM/MeOH (10/1). The crude product was purified by Prep-HPLC with the following conditions (Column: XBridge Prep OBD C18 Column, 30*150 mm, 5 μm; Mobile Phase A: water (0.1% NH₄HCO₃+0.1% NH₃·H₂O), Mobile Phase B: MeCN; Flow rate: 60 mL/min; Gradient: 65% B to 75% B in 8 min; Wave Length: 254/220 nm; RT: 7.47) to afford the title compound (AGD-1) (20.3 mg, 35 μmol, 48%, 99.8% Purity) as a white solid. m/z 573.1 (M+H)⁺ (ES+). ¹H NMR (300 MHz, DMSO-d6) δ 8.50 (s, 1H), 7.89-7.75 (m, 3H), 7.68-7.60 (m, 2H), 7.14 (d, J=1.6 Hz, 1H), 3.79 (s, 2H), 3.31 (s, 3H), 2.21-2.14 (m, 1H), 1.91-1.64 (m, 7H), 1.08-0.92 (m, 10H).

### Example 436: 1-Cyclobutyl-N-((2-(6-cyclopropyl-4-(4-fluoro-2-(4-methyl-4H-1,2,4-triazol-3-yl)phenyl)pyridin-2-yl)-6,7-difluorobenzo[d]oxazol-5-yl)methyl)methanamine (AGE-1)

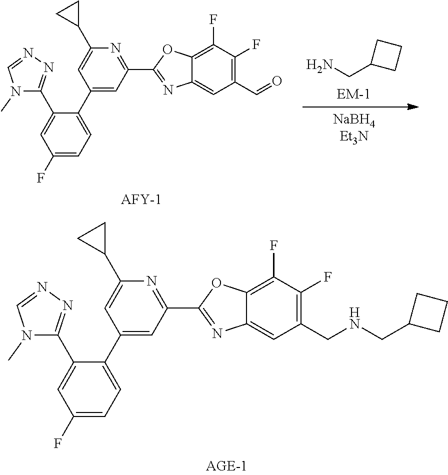

To a stirred mixture of intermediate (AFY-1) (35 mg, 1 Eq, 74 μmol) and 1-cyclobutylmethanamine (EM-1) (13 mg, 2 Eq, 0.15 mmol) in MeOH (5 mL) was added Et₃N (22 mg, 3 Eq, 0.22 mmol) at rt. The resulting mixture was stirred for 2 h at 60° C. The mixture was allowed to cool down to rt. To the above mixture was added NaBH₄ (5 mg, 1.8 Eq, 0.13 mmol) at rt. The resulting mixture was stirred for additional 2 h at rt. The reaction was then quenched by the addition of 2 mL of MeOH at 0° C. The resulting mixture was concentrated in vacuo. The residue was purified by Prep-TLC with DCM/MeOH (10/1). The crude product was purified by Prep-HPLC with the following conditions (Column: XBridge Prep OBD C18 Column, 30*150 mm, 5 μm; Mobile Phase A: water (0.1% NH₄HCO₃+0.1% NH₃·H₂O), Mobile Phase B: MeCN; Flow rate: 60 mL/min; Gradient: 50% B to 62% B in 9 min; Wave Length: 254/220 nm; RT: 8.08) to afford the title compound (AGE-1) (8.5 mg, 16 μmol, 21%, 98.8% Purity) as a white solid. m/z 545.1 (M+H)⁺ (ES+). ¹H NMR (300 MHz, DMSO-d6) δ 8.50 (s, 1H), 7.89-7.81 (m, 1H), 7.81-7.72 (m, 2H), 7.68-7.59 (m, 2H), 7.13 (d, J=1.6 Hz, 1H), 3.85 (s, 2H), 3.30 (s, 3H), 2.54 (d, J=7.1 Hz, 2H), 2.43-2.36 (m, 1H), 2.21-2.14 (m, 1H), 2.04-1.95 (m, 2H), 1.86-1.76 (m, 2H), 1.70-1.59 (m, 2H), 1.09-1.01 (m, 2H), 0.99-0.90 (m, 2H).

### Example 437: 1-{[(2-{6-Cyclopropyl-4-[4-fluoro-2-(4-methyl-1,2,4-triazol-3-yl)phenyl]pyridin-2-yl}-6,7-difluoro-1,3-benzoxazol-5-yl)methyl]amino}-2-methylpropan-2-ol (AGF-1)

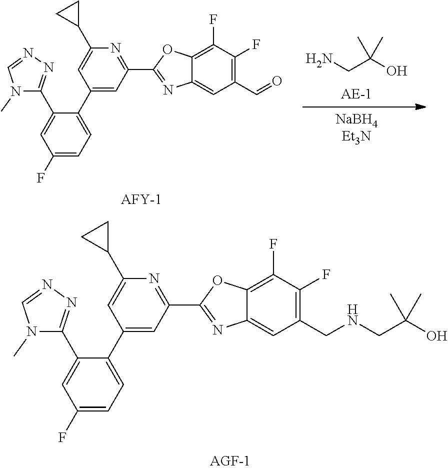

To a stirred mixture of intermediate (AFY-1) (35 mg, 1 Eq, 74 μmol) and 1-amino-2-methylpropan-2-ol (AE-1) (13 mg, 2 Eq, 0.15 mmol) in MeOH (5 mL) was added Et₃N (22 mg, 3 Eq, 0.22 mmol) at rt. The resulting mixture was stirred for 2 h at 60° C. The mixture was allowed to cool down to rt. To the above mixture was added NaBH₄ (14 mg, 5 Eq, 0.37 mmol) at rt. The resulting mixture was stirred for additional 2 h at rt. The reaction was then quenched by the addition of 2 mL of MeOH at 0° C. The resulting mixture was concentrated in vacuo. The residue was purified by Prep-TLC with DCM/MeOH (10/1). The crude product was purified by Prep-HPLC with the following conditions (Column: XBridge Prep OBD C18 Column, 30*150 mm, 5 μm; Mobile Phase A: water (0.1% NH₄HCO₃+0.1% NH₃·H₂O), Mobile Phase B: MeCN; Flow rate: 60 mL/min; Gradient: 37% B to 47% B in 9 min; Wave Length: 254/220 nm; RT: 8.02) to afford the title compound (AGF-1) (15.8 mg, 29 μmol, 39%, 99.0% Purity) as a white solid. m/z 549.0 (M+H)⁺ (ES+). ¹H NMR (300 MHz, Methanol-d4) δ 8.46 (s, 1H), 7.90 (d, J=1.6 Hz, 1H), 7.87-7.80 (m, 1H), 7.74-7.68 (m, 1H), 7.62-7.48 (m, 2H), 7.11 (d, J=1.6 Hz, 1H), 4.06-3.98 (m, 2H), 3.29 (s, 3H), 2.57 (s, 2H), 2.22-2.12 (m, 1H), 1.22 (s, 6H), 1.12-1.00 (m, 4H).

### Example 438: 4-[2-Cyclopropyl-6-(5-{[(3S)-3-methylpiperidin-1-yl]methyl}-1,3-benzoxazol-2-yl)pyridin-4-yl]-3-(4-methyl-1,2,4-triazol-3-yl)benzonitrile (AGG-2)

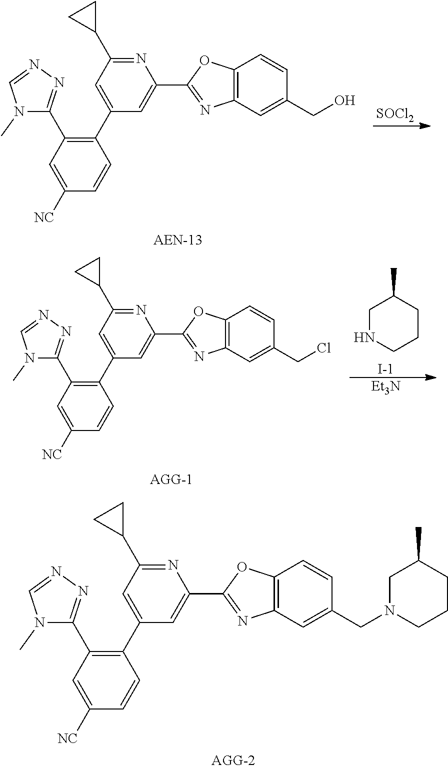

Step 1: 4-(2-(5-(Chloromethyl)benzo[d]oxazol-2-yl)-6-cyclopropylpyridin-4-yl)-3-(4-methyl-4H-1,2,4-triazol-3-yl)benzonitrile (AGG-1). To a stirred solution of intermediate (AEN-13) (25 mg, 1 Eq, 56 μmol) and SOCl₂ (0.05 mL) in DCM (1.5 mL) at 0° C. The resulting mixture was stirred for 1 h at 0° C. The reaction was then quenched by the addition of 2 mL of ice water at 0° C. The resulting mixture was concentrated in vacuo. The crude product used directly in next step without any further purification. m/z 467.1/469.1 (M+H)⁺ (ES+).

Step 2: 4-[2-Cyclopropyl-6-(5-{[(3S)-3-methylpiperidin-1-yl]methyl}-1,3-benzoxazol-2-yl)pyridin-4-yl]-3-(4-methyl-1,2,4-triazol-3-yl)benzonitrile (AGG-2). To a stirred solution of the product from step 1 above (AGG-1) (25 mg, 1 Eq, 54 μmol) and (3S)-3-methylpiperidine (I-1) (11 mg, 2 Eq, 0.11 mmol) in MeOH (1.5 mL) was added Et₃N (27 mg, 5 Eq, 0.27 mmol) at rt. The resulting mixture was stirred for 2 h at rt. The resulting mixture was diluted with water and extracted with DCM (3×10 mL). The combined organic layers were washed with brine (2×10 mL), dried (Na₂SO₄). After filtration, the filtrate was concentrated in vacuo. The crude product was purified by Prep-HPLC with the following conditions: Column: XBridge Prep OBD C18 Column, 30×150 mm, 5 μm; Mobile Phase A: water (0.1% NH₄HCO₃+0.1% NH₃·H₂O) Mobile Phase B: MeCN; Flow rate: 60 mL/min; Gradient: 51 B to 61 B in 8 min; Detector, UV 254/220 nm; RT: 8.19. The product-containing fractions were combined and evaporated partially in vacuo and lyophilized overnight to afford the title compound (AGG-2) (10.9 mg, 21 μmol, 38%, 99.9% Purity) as a white solid. m/z 530.3 (M+H)⁺ (ES+). ¹H NMR (400 MHz, Methanol-d4) δ 8.49 (s, 1H), 8.20-8.10 (m, 2H), 8.01-7.94 (m, 1H), 7.91 (d, J=1.6 Hz, 1H), 7.78-7.73 (m, 1H), 7.68 (d, J=8.4 Hz, 1H), 7.50-7.45 (m, 1H), 7.17 (d, J=1.6 Hz, 1H), 3.65 (s, 2H), 3.33 (s, 3H), 2.95-2.79 (m, 2H), 2.23-2.13 (m, 1H), 2.06-1.90 (m, 1H), 1.78-1.58 (m, 5H), 1.13-1.02 (m, 4H), 0.93-0.81 (m, 4H).

### Example 439: 4-(2-Cyclopropyl-6-{7-fluoro-5-[(1S,4S)-2-oxa-5-azabicyclo[2.2.1]heptan-5-ylmethyl]-1,3-benzoxazol-2-yl}pyridin-4-yl)-3-(4-methyl-1,2,4-triazol-3-yl)benzonitrile (AGH-4)

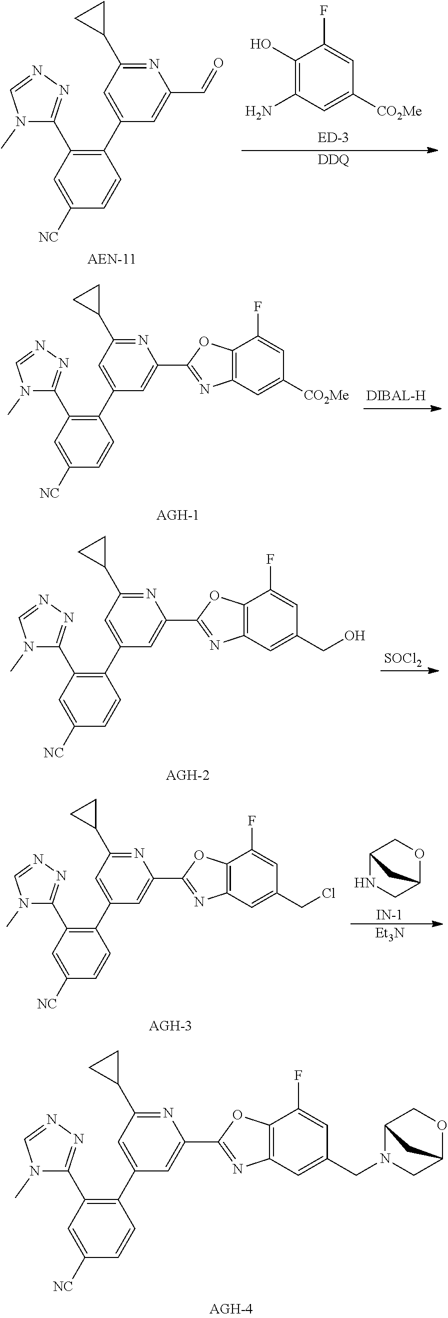

Step 1: Methyl 2-{4-[4-cyano-2-(4-methyl-1,2,4-triazol-3-yl)phenyl]-6-cyclopropylpyridin-2-yl}-7-fluoro-1,3-benzoxazole-5-carboxylate (AGH-1). A solution of intermediate (AEN-11) (120 mg, 1 Eq, 0.36 mmol) and intermediate (ED-3) (101 mg, 1.5 Eq, 0.55 mmol) in DCM (15 mL) was stirred for 1 h at 60° C. The mixture was allowed to cool down to rt. To the above mixture was added DDQ (165 mg, 2 Eq, 0.73 mmol) at rt. The resulting mixture was stirred for 2 h at rt. The resulting mixture was diluted with water and extracted with EtOAc (3×50 mL). The combined organic layers were washed with brine (2×50 mL), dried (Na₂SO₄). After filtration, the filtrate was concentrated in vacuo. The crude product was purified by reverse flash column Chromatography with the following conditions: Column, C18; mobile phase, water (0.1% FA) and MeCN (35% MeCN up to 60% in 20 min); Detector, UV 254/220 nm. The product-containing fractions were combined and concentrated in vacuo. This resulted in the sub-title compound (AGH-1) (120 mg, 0.24 mmol, 67%, 90% Purity) as a yellow solid. m/z 495.2 (M+H)⁺ (ES+).

Step 2: 4-{2-Cyclopropyl-6-[7-fluoro-5-(hydroxymethyl)-1,3-benzoxazol-2-yl]pyridin-4-yl}-3-(4-methyl-1,2,4-triazol-3-yl)benzonitrile (AGH-2). To a stirred solution of the product from step 1 above (AGH-1) (15 mg, 1 Eq, 0.03 mmol) in THF (1.5 mL) was added DIBAL-H (g) in THF (0.3 mL, 1 M, 10 Eq, 0.3 mmol) was stirred for 15 minutes at 0° C. under nitrogen atmosphere. The reaction was then quenched by the addition of 1 mL of ice water at 0° C. The resulting mixture was diluted with water and extracted with EtOAc (3×10 mL). The combined organic layers were washed with brine (2×10 mL), dried (Na₂SO₄). After filtration, the filtrate was concentrated in vacuo. The residue was purified by Prep-TLC with DCM/MeOH (10/1). This resulted in the sub-title compound (AGH-2) (10 mg, 21 μmol, 71%, 95% Purity) as a white solid. m/z 467.2 (M+H)⁺ (ES+).

Step 3: 4-{2-[5-(Chloromethyl)-7-fluoro-1,3-benzoxazol-2-yl]-6-cyclopropylpyridin-4-yl}-3-(4-methyl-1,2,4-triazol-3-yl)benzonitrile (AGH-3). To a stirred solution of the product from step 2 above (AGH-2) (70 mg, 1 Eq, 0.15 mmol) in DCM (5 mL) was added SOCl₂ (21 mg, 1.2 Eq, 0.18 mmol) at 0° C. The resulting mixture was stirred for 1 h at 0° C. The reaction was then quenched by the addition of 2 mL of MeOH at 0° C. The resulting mixture was concentrated in vacuo affording the sub-title compound (AGH-3) (60 mg, 0.12 mmol, 82%, 83% Purity) as a yellow solid, which was used directly without purification. m/z 485.1/487.1 (M+H)⁺ (ES+).

Step 4: 4-(2-Cyclopropyl-6-{7-fluoro-5-[(1S,4S)-2-oxa-5-azabicyclo[2.2.1]heptan-5-ylmethyl]-1,3-benzoxazol-2-yl}pyridin-4-yl)-3-(4-methyl-1,2,4-triazol-3-yl)benzonitrile (AGH-4). To a stirred solution of the product from step 3 above (AGH-3) (30 mg, 1 Eq, 62 μmol) and (1S,4S)-2-oxa-5-azabicyclo[2.2.1]heptane (IN-1) (9 mg, 1.5 Eq, 93 μmol) in DCM (4 mL) was added Et₃N (31 mg, 5 Eq, 0.31 mmol) at rt. The resulting mixture was stirred for overnight at rt at 60° C. The mixture was allowed to cool down to rt. The resulting mixture was diluted with water and extracted with DCM (3×20 mL). The combined organic layers were washed with brine (2×20 mL), dried (Na₂SO₄). After filtration, the filtrate was concentrated in vacuo. The crude product was purified by Prep-HPLC with the following conditions: Column: XBridge Prep OBD C18 Column, 30×150 mm, 5 μm; Mobile Phase A: water (0.1% NH₄HCO₃+0.1% NH₃·H2O) Mobile Phase B: MeCN; Flow rate: 60 mL/min; Gradient: 34 B to 44 B in 8 min; Detector, UV 254/210 nm; RT: 7.32. The product-containing fractions were combined and evaporated partially in vacuo and lyophilized overnight to afford the title compound (AGH-4) (6.4 mg, 12 μmol, 18%, 97.3% Purity) as a white solid. m/z 548.2 (M+H)⁺ (ES+). ¹H NMR (400 MHz, DMSO-d6) δ 8.53 (s, 1H), 8.28-8.21 (m, 2H), 8.03-7.95 (m, 1H), 7.84 (d, J=1.5 Hz, 1H), 7.65 (s, 1H), 7.40 (d, J=11.3 Hz, 1H), 7.19 (d, J=1.5 Hz, 1H), 4.37 (s, 1H), 3.95 (d, J=7.5 Hz, 1H), 3.91-3.80 (m, 2H), 3.56-3.51 (m, 1H), 3.51-3.46 (m, 1H), 3.35 (s, 3H), 2.80-2.74 (m, 1H), 2.44 (d, J=9.9 Hz, 1H), 2.21-2.16 (m, 1H), 1.86-1.80 (m, 1H), 1.61 (d, J=9.3 Hz, 1H), 1.10-1.02 (m, 2H), 0.99-0.91 (m, 2H).

### Example 440: 4-[2-Cyclopropyl-6-(7-fluoro-5-{[(3S)-3-methylpiperidin-1-yl]methyl}-1,3-benzoxazol-2-yl)pyridin-4-yl]-3-(4-methyl-1,2,4-triazol-3-yl)benzonitrile (AGI-1)

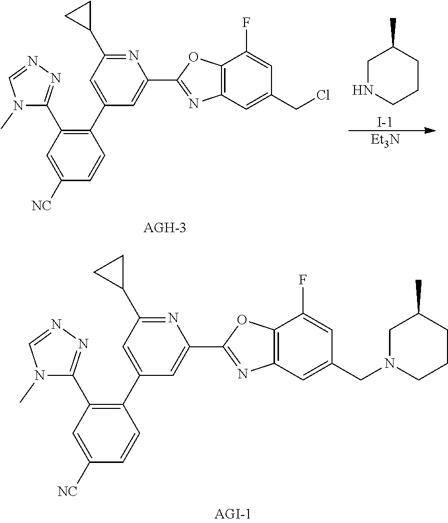

To a stirred solution of intermediate (AGH-3) (30 mg, 1 Eq, 62 μmol) and (3S)-3-methylpiperidine (I-1) (9 mg, 1.5 Eq, 93 μmol) in DCM (4 mL) was added Et₃N (31 mg, 5 Eq, 0.31 mmol) at rt. The resulting mixture was stirred for overnight at rt at 60° C. The mixture was allowed to cool down to rt. The resulting mixture was diluted with water and extracted with DCM (3×20 mL). The combined organic layers were washed with brine (2×20 mL), dried (Na₂SO₄). After filtration, the filtrate was concentrated in vacuo. The crude product was purified by Prep-HPLC with the following conditions: Column: XBridge Prep OBD C18 Column, 30×150 mm, 5 μm; Mobile Phase A: water (0.1% NHLHCO₃+0.1% NH₃·H₂O) Mobile Phase B: MeCN; Flow rate: 60 mL/min; Gradient: 55 B to 65 B in 9 min; Detector, UV 254/210 nm; RT: 8.7. The product-containing fractions were combined and evaporated partially in vacuo and lyophilized overnight to afford the title compound (AGI-1) (6.9 mg, 13 μmol, 20%, 99.8% Purity) as a white solid. m/z 548.2 (M+H)⁺ (ES+). ¹H NMR (400 MHz, DMSO-d6) δ 8.53 (s, 1H), 8.29-8.18 (m, 2H), 8.07-7.92 (m, 1H), 7.84 (d, J=1.5 Hz, 1H), 7.60 (d, J=1.2 Hz, 1H), 7.35 (d, J=11.3 Hz, 1H), 7.19 (d, J=1.6 Hz, 1H), 3.57 (s, 2H), 3.35 (s, 2H), 3.33 (s, 1H), 2.74 (t, J=8.7 Hz, 2H), 2.26-2.14 (m, 1H), 1.90 (t, J=10.7 Hz, 1H), 1.70-1.45 (m, 4H), 1.23 (s, 1H), 1.09-0.97 (m, 2H), 0.97-0.87 (m, 2H), 0.82 (d, J=5.4 Hz, 4H).

### Example 441: [(2-{6-Cyclopropyl-4-[4-fluoro-2-(4-methyl-1,2,4-triazol-3-yl)phenyl]pyridin-2-yl}-7-methyl-1,3-benzoxazol-5-yl)methyl](1-methoxy-2-methylpropan-2-yl)amine (AGJ-1)

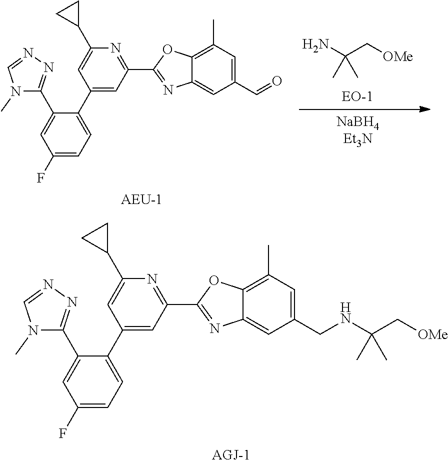

A solution of intermediate (AEU-1) (30 mg, 1 Eq, 66 μmol) and 1-methoxy-2-methylpropan-2-amine (EO-1) (10 mg, 1.5 Eq, 99 μmol) in MeOH (5 mL) was added Et₃N (33 mg, 5 Eq, 0.33 mmol) for 2 h at 60° C. under nitrogen atmosphere. The mixture was allowed to cool down to rt. To the above mixture was added NaBH₄ (25 mg, 10 Eq, 0.66 mmol) at 0° C. The resulting mixture was stirred for 1 h at rt. The reaction was then quenched by the addition of 2 mL of MeOH at 0° C. The resulting mixture was diluted with water and extracted with DCM (3×10 mL). The combined organic layers were washed with brine (2×10 mL), dried (Na₂SO₄). After filtration, the filtrate was concentrated in vacuo. The crude product was purified by Prep-HPLC with the following conditions: Column: XBridge Prep OBD C18 Column, 30×150 mm, 5 μm; Mobile Phase A: water (0.1% NH₄HCO₃+0.1% NH₃·H₂O) Mobile Phase B: MeCN; Flow rate: 60 mL/min; Gradient: 55 B to 65 B in 9 min; Detector, UV 254/210 nm; RT: 8.7. The product-containing fractions were combined and evaporated partially in vacuo and lyophilized overnight to afford the title compound (AGJ-1) (5.6 mg, 10 μmol, 16%, 99.2% Purity) as a white solid. m/z 541.7 (M+H)⁺ (ES+). ¹H NMR (400 MHz, DMSO-d6) δ 8.51 (s, 1H), 7.89-7.84 (m, 1H), 7.73 (d, J=1.5 Hz, 1H), 7.66-7.60 (m, 2H), 7.57 (s, 1H), 7.27 (s, 1H), 7.08 (d, J=1.6 Hz, 1H), 3.74 (s, 2H), 3.30 (s, 6H), 3.21 (s, 2H), 2.54 (s, 3H), 2.21-2.14 (m, 1H), 1.10-0.99 (m, 8H), 0.96-0.89 (m, 2H).

### Example 442: [(2-{6-Cyclopropyl-4-[4-fluoro-2-(4-methyl-1,2,4-triazol-3-yl)phenyl]pyridin-2-yl}-7-methyl-1,3-benzoxazol-5-yl)methyl][(2S)-1-methoxypropan-2-yl]amine (AGK-1)

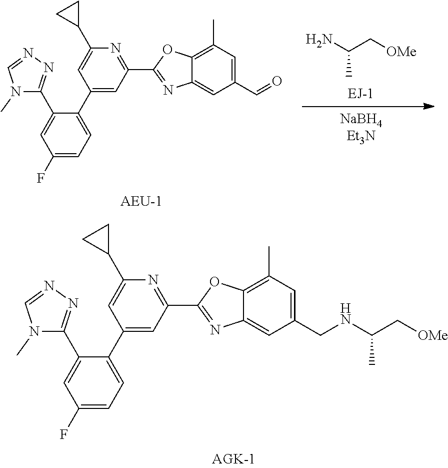

To a stirred solution of intermediate (AEU-1) (25 mg, 1 Eq, 55 μmol) and (2S)-1-methoxypropan-2-amine (EJ-1) (5 mg, 1 Eq, 55 μmol) in DCM (2 mL) was added Et₃N (28 mg, 5 Eq, 0.28 mmol) for 2 h at 60° C. under nitrogen atmosphere. The mixture was allowed to cool down to rt. To the above mixture was added NaBH₄ (21 mg, 10 Eq, 0.55 mmol) at 0° C. The resulting mixture was stirred for 2 h at rt. The reaction was then quenched by the addition of 2 mL of MeOH at 0° C. The resulting mixture was diluted with water and extracted with DCM (3×10 mL). The combined organic layers were washed with brine (2×10 mL), dried (Na₂SO₄). After filtration, the filtrate was concentrated in vacuo. The crude product was purified by Prep-HPLC with the following conditions: Column: XBridge Prep OBD C18 Column, 19×250 mm, 5 μm; Mobile Phase A: water (0.1% NH₄HCO₃) Mobile Phase B: MeCN; Flow rate: 25 mL/min; Gradient: 42 B to 52 B in 8 min; Detector, UV 254/210 nm; RT: 5.7. The product-containing fractions were combined and evaporated partially in vacuo and lyophilized overnight to afford the title compound (AGK-1) (2.4 mg, 4.6 μmol, 8.2%, 98.9% Purity) as a white solid. m/z 527.3 (M+H)⁺ (ES+). ¹H NMR (300 MHz, Methanol-d4) δ 8.46 (s, 1H), 8.11-7.80 (m, 2H), 7.79-7.39 (m, 3H), 7.30 (s, 1H), 7.10 (d, J=1.6 Hz, 1H), 3.99 (d, J=13.1 Hz, 1H), 3.86 (d, J=13.2 Hz, 1H), 3.34 (s, 4H), 3.30 (s, 4H), 3.04-2.88 (m, 1H), 2.62 (t, J=0.7 Hz, 3H), 2.25-2.12 (m, 1H), 1.46-0.76 (m, 7H).

### Example 443: [(2-{6-Cyclopropyl-4-[4-fluoro-2-(4-methyl-1,2,4-triazol-3-yl)phenyl]pyridin-2-yl}-7-methyl-1,3-benzoxazol-5-yl)methyl][(2R)-1-methoxypropan-2-yl]amine (AGL-1)

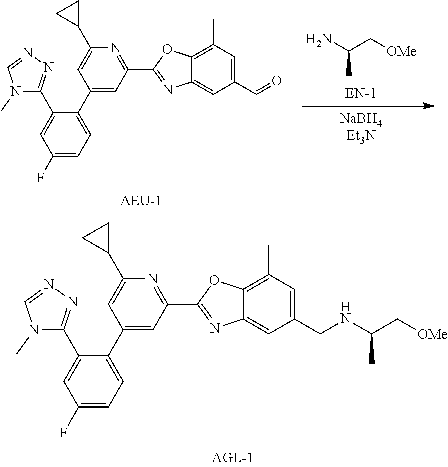

To a stirred solution of intermediate (AEU-1) (25 mg, 1 Eq, 55 μmol) and (2R)-1-methoxypropan-2-amine (EN-1) (5 mg, 1 Eq, 55 μmol) in MeOH (1.5 mL) was added Et₃N (28 mg, 5 Eq, 0.28 mmol) for 2 h at 60° C. under nitrogen atmosphere. The mixture was allowed to cool down to rt. To the above mixture was added NaBH₄ (21 mg, 10 Eq, 0.55 mmol) at 0° C. The resulting mixture was stirred for 2 h at rt. The reaction was then quenched by the addition of 2 mL of MeOH at 0° C. The resulting mixture was diluted with water and extracted with DCM (3×10 mL). The combined organic layers were washed with brine (2×10 mL), dried (Na₂SO₄). After filtration, the filtrate was concentrated in vacuo. The crude product was purified by Prep-HPLC with the following conditions: Column: XBridge Prep OBD C18 Column, 30×150 mm, 5 μm; Mobile Phase A: water (0.1% NH₄HCO₃) Mobile Phase B: MeCN; Flow rate: 60 mL/min; Gradient: 36 B to 46 B in 8 min; Detector, UV 254/220 nm; RT: 5.7. The product-containing fractions were combined and evaporated partially in vacuo and lyophilized overnight to afford the title compound (AGL-1) (3.4 mg, 6.5 μmol, 14%, 99.6% Purity) as a white solid. m/z 527.1 (M+H)⁺ (ES+). ¹H NMR (400 MHz, Methanol-d4) δ 8.47 (s, 1H), 7.90-7.82 (m, 2H), 7.62-7.55 (m, 2H), 7.54-7.49 (m, 1H), 7.29 (t, J=1.2 Hz, 1H), 7.10 (d, J=1.6 Hz, 1H), 3.96-3.83 (m, 2H), 3.41-3.33 (m, 5H), 3.32 (s, 3H), 2.97-2.90 (m, 1H), 2.61 (s, 3H), 2.20-2.14 (m, 1H), 1.12-1.04 (m, 7H).

### Example 444: Methyl 1-(2-{6-cyclopropyl-4-[4-fluoro-2-(4-methyl-1,2,4-triazol-3-yl) phenyl]pyridin-2-yl}-7-fluoro-1,3-benzoxazol-5-yl) cyclopropane-1-carboxylate (AGM-8)

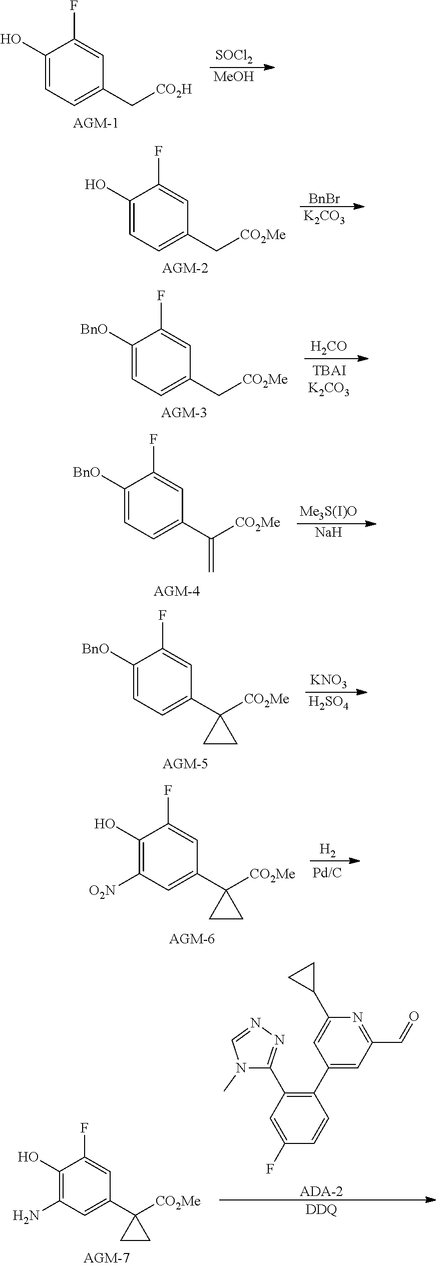

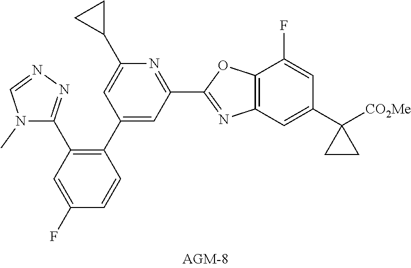 [SMILES: COC(=O)C1(c2cc(F)c3oc(-c4cc(-c5ccc(F)cc5-c5nncn5C)cc(C5CC5)n4)nc3c2)CC1]

Step 1: Methyl 2-(3-fluoro-4-hydroxyphenyl)acetate (AGM-2). To a stirred solution of (3-fluoro-4-hydroxyphenyl)acetic acid (AGM-1) (5.00 g, 1 Eq, 29.4 mmol) in MeOH (20 mL) was added SOCl₂ (10.5 g, 3 Eq, 88.2 mmol) at rt. The resulting mixture was stirred for 2 h at 80° C. The mixture was allowed to cool down to rt. The reaction was then quenched by the addition of 10 mL of MeOH at 0° C. The residue was applied on a silica gel column chromatography with petroleum ether/EtOAc (5/1) to afford the sub-title compound (AGM-2) (4.5 g, 24.4 mmol, 83%, 90% Purity) as a yellow solid. m/z 185.1 (M+H)⁺ (ES+)

Step 2: Methyl 2-[4-(benzyloxy)-3-fluorophenyl]acetate (AGM-3). To a stirred solution of the product from step 1 above (AGM-2) (4.5 g, 1 Eq, 24.4 mmol) and benzyl bromide (6.3 g, 1.5 Eq, 36.7 mmol) in DMF (20 mL) was added K₂CO₃ (6.75 g, 2 Eq, 48.9 mmol) at rt. The resulting mixture was stirred for 2 h at 60° C. The mixture was cooled to rt, diluted with water and extracted with EtOAc (3×200 mL). The combined organic layers were washed with brine (2×200 mL), dried (Na₂SO₄). After filtration, the filtrate was concentrated in vacuo. The residue was applied on a silica gel column chromatography with petroleum ether/EtOAc (5/1) to afford the sub-title compound (AGM-3) (4.3 g, 15.7 mmol, 64%, 92% Purity) as a colorless oil. m/z 275.1 (M+H)⁺ (ES+).

Step 3: Methyl 2-[4-(benzyloxy)-3-fluorophenyl]prop-2-enoate (AGM-4). To a stirred solution of the product from step 2 above (AGM-3) (4.3 g, 1 Eq, 15.7 mmol) and formaldehyde (2.35 g, 5 Eq, 78.4 mmol) in toulene (20 mL) were added K₂CO₃ (6.5 g, 3 Eq, 47.0 mmol) and TBAI (290 mg, 0.05 Eq, 0.78 mmol) at rt. The resulting mixture was stirred for 4 h at 80° C. and was then stirred for 16 h at rt. The mixture was cooled to rt, diluted with water and extracted with EtOAc (3×200 mL). The combined organic layers were washed with brine (2×200 mL), dried (Na₂SO₄). After filtration, the filtrate was concentrated in vacuo. The residue was applied on a silica gel column chromatography with petroleum ether/EtOAc (6/1) to afford the sub-title compound (AGM-4) (1.5 g, 5.24 mmol, 33%, 92% Purity) as a colourless oil. m/z 287.1 (M+H)⁺ (ES+).

Step 4: Methyl 1-[4-(benzyloxy)-3-fluorophenyl]cyclopropane-1-carboxylate (AGM-5). A solution of trimethylsulfoxonium iodide (2.3 g, 10.478 mmol, 2.0 Eq) in DMF (20 mL) was treated with NaH (252 mg, 60% Wt, 2 Eq, 10.5 mmol) for 30 mins at 0° C. under nitrogen atmosphere followed by the addition of the product from step 3 above (AGM-4) (1.5 g, 1 Eq, 5.24 mmol) at rt under nitrogen atmosphere. The resulting mixture was stirred for 2 h at rt under nitrogen atmosphere. The reaction was then quenched by the addition of 10 mL of ice water at 0° C. The resulting mixture was diluted with water and extracted with EtOAc (3×100 mL). The combined organic layers were washed with brine (2×100 mL), dried (Na₂SO₄). After filtration, the filtrate was concentrated in vacuo. The residue was purified by Prep-TLC with EtOAc/petroleum ether (1/6) to afford the sub-title compound (AGM-5) (700 mg, 2.33 mmol, 44%, 90% Purity) as a colorless oil. m/z 301.1 (M+H)⁺ (ES+).

Step 5: Methyl 1-(3-fluoro-4-hydroxy-5-nitrophenyl)cyclopropane-1-carboxylate (AGM-6). To a stirred solution of the product from step 4 above (AGM-5) (700 mg, 1 Eq, 2.33 mmol) in conc. H₂SO₄ (30 mL) was added KNO₃ (259 mg, 1.1 Eq, 2.56 mmol) in portions at 0° C. The resulting mixture was stirred for 0.5 h at rt. The resulting mixture was diluted with water and extracted with EtOAc (3×80 mL). The combined organic layers were washed with brine (2×80 mL), dried (Na₂SO₄). After filtration, the filtrate was concentrated in vacuo. The residue was purified by Prep-TLC with DCM/MeOH (8/1) to afford the sub-title compound (AGM-6) (350 mg, 1.37 mmol, 59%, 93% Purity) as a yellow solid. m/z 256.1 (M+H)⁺ (ES+).

Step 6: 6-Cyclopropyl-4-[4-fluoro-2-(4-methyl-1,2,4-triazol-3-yl)phenyl]pyridine-2-carbaldehyde (AGM-7). To a solution of the product from step 5 above (AGM-6) (350 mg, 1 Eq, 1.37 mmol) in MeOH (10 Ml) was added Pd/C (145 mg, 10% Wt, 10 Eq, 13.7 mmol) under nitrogen atmosphere. The resulting mixture was stirred for 12 h at rt under hydrogen atmosphere. The resulting mixture was filtered the filter cake was washed with EtOAc (3×5 mL). The filtrate was concentrated in vacuo. The crude product was used in the next step directly without further purification. m/z 226.1 (M+H)⁺ (ES+).

Step 7: Methyl 1-(2-{6-cyclopropyl-4-[4-fluoro-2-(4-methyl-1,2,4-triazol-3-yl) phenyl]pyridin-2-yl}-7-fluoro-1,3-benzoxazol-5-yl) cyclopropane-1-carboxylate (AGM-8). A solution of intermediate (ADA-3) (140 mg, 1 Eq, 0.62 mmol) and the product from step 6 above (AGM-7) (200 mg, 1 Eq, 0.62 mmol) in DCM (15 mL) was stirred for 1 h at 60° C. The mixture was allowed to cool down to rt. To the above mixture was added DDQ (282 mg, 2 Eq, 1.24 mmol) at rt. The resulting mixture was stirred for additional 1 h at rt. The resulting mixture was diluted with water and extracted with EtOAc (3×80 mL). The combined organic layers were washed with brine (2×80 mL), dried (Na₂SO₄). After filtration, the filtrate was concentrated in vacuo. The residue was purified by Prep-TLC with DCM/MeOH (8/1). The crude product was purified by Prep-HPLC with the following conditions (Column: Xselect CSH C18 OBD Column 30*150 mm, 5 μm; Mobile Phase A: water (0.1% FA), Mobile Phase B: MeCN; Flow rate: 60 mL/min; Gradient: 35% B to 65% B in 10 min; Wave Length: 254 nm) to afford the title compound (AGM-8) (2.2 mg, 4.2 μmol, 0.54%, 99.8% Purity) as a white solid. m/z 528.1 (M+H)⁺ (ES+). ¹H NMR (400 MHz, DMSO-d6) δ 8.50 (s, 1H), 7.89-7.83 (m, 1H), 7.78 (d, J=1.6 Hz, 1H), 7.69 (d, J=1.4 Hz, 1H), 7.67-7.61 (m, 2H), 7.52-7.45 (m, 1H), 7.14 (d, J=1.6 Hz, 1H), 3.57 (s, 3H), 3.32 (s, 3H), 2.23-2.14 (m, 1H), 1.58-1.54 (m, 2H), 1.39-1.33 (m, 2H), 1.09-1.03 (m, 2H), 0.98-0.92 (m, 2H).

### Example 445: 1-(2-{6-Cyclopropyl-4-[4-fluoro-2-(4-methyl-1,2,4-triazol-3-yl)phenyl]pyridin-2-yl}-7-fluoro-1,3-benzoxazol-5-yl) cyclopropan-1-amine (AGN-3)

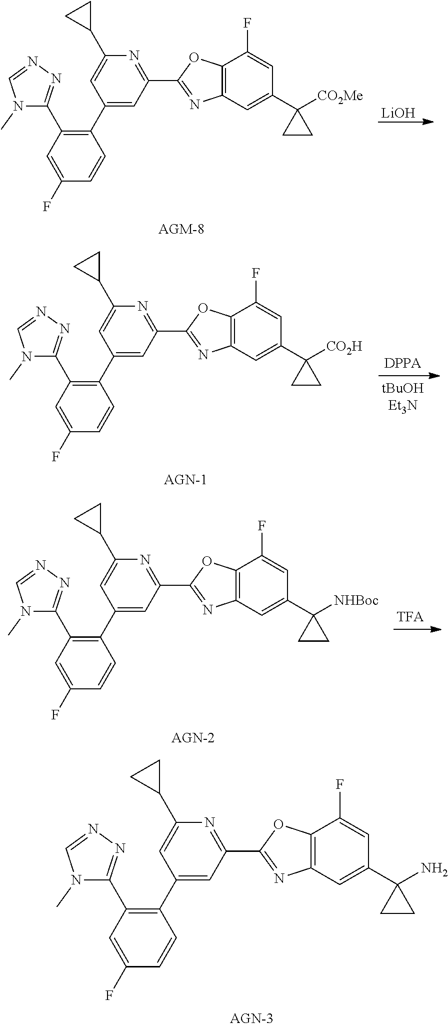 [SMILES: COC(=O)C1(c2cc(F)c3oc(-c4cc(-c5ccc(F)cc5-c5nncn5C)cc(C5CC5)n4)nc3c2)CC1 ; "LiOH" ; Cn1cnnc1-c1cc(F)ccc1-c1cc(-c2nc3cc(C4(C(=O)O)CC4)cc(F)c3o2)nc(C2CC2)c1 ; Cn1cnnc1-c1cc(F)ccc1-c1cc(-c2nc3cc(C4(NC(=O)OC(C)(C)C)CC4)cc(F)c3o2)nc(C2CC2)c1 ; Cn1cnnc1-c1cc(F)ccc1-c1cc(-c2nc3cc(C4(N)CC4)cc(F)c3o2)nc(C2CC2)c1]

Step 1: 1-(2-{6-Cyclopropyl-4-[4-fluoro-2-(4-methyl-1,2,4-triazol-3-yl) phenyl]pyridin-2-yl}-7-fluoro-1,3-benzoxazol-5-yl) cyclopropane-1-carboxylic acid (AGN-1). To a stirred solution of compound (AGM-8) (200 mg, 1 Eq, 0.38 mmol) in THF (20 mL) and water (4 mL) was added LiOH (45 mg, 5 Eq, 1.90 mmol) at rt. The resulting mixture was stirred for 3 h at 60° C. The mixture was cooled to rt and concentrated in vacuo. The pH value of the solution was adjusted to 3 with aq. of HCl (1 M) at 0° C. The resulting mixture was diluted with water and extracted with EtOAc (3×50 mL). The combined organic layers were washed with brine (2×50 mL), dried (Na₂SO₄). After filtration, the filtrate was concentrated in vacuo. The crude product was used in the next step directly without further purification. m/z 514.2 (M+H)⁺ (ES+).

Step 2: tert-Butyl N-[1-(2-{6-cyclopropyl-4-[4-fluoro-2-(4-methyl-1,2,4-triazol-3-yl) phenyl]pyridin-2-yl}-7-fluoro-1,3-benzoxazol-5-yl) cyclopropyl]carbamate (AGN-2). To a stirred solution of the product from step 1 above (AGN-1) (190 mg, 1 Eq, 0.37 mmol) in t-BuOH (10 mL) were added Et₃N (75 mg, 2 Eq, 0.74 mmol) and DPPA (122 mg, 1.2 Eq, 0.44 mmol) at rt under nitrogen atmosphere. The resulting mixture was stirred for 1 h at rt under nitrogen atmosphere. The mixture was stirred for 12 h at 80° C. under nitrogen atmosphere. The mixture was allowed to cool down to rt. The resulting mixture was diluted with water and extracted with EtOAc (3×50 mL). The combined organic layers were washed with brine (2×50 mL), dried (Na₂SO₄). After filtration, the filtrate was concentrated in vacuo. The residue was purified by Prep-TLC with DCM/MeOH (8/1) to afford the sub-title compound (AGN-2) (110 mg, 51%, 92% Purity) as a brown solid. m/z 585.2 (M+H)⁺ (ES+).

Step 3: 1-(2-{6-Cyclopropyl-4-[4-fluoro-2-(4-methyl-1,2,4-triazol-3-yl) phenyl]pyridin-2-yl}-7-fluoro-1,3-benzoxazol-5-yl) cyclopropan-1-amine (AGN-3). To a stirred solution of the product from step 2 above (AGN-2) (20 mg, 1 Eq, 34 μmol) in DCM (6 mL) were added TFA (2 mL) at 0° C. The resulting mixture was stirred for 1 h at rt. The resulting mixture was concentrated in vacuo. The crude product was purified by Prep-HPLC with the following conditions (Column: Xselect CSH C18 OBD Column 30*150 mm, 5 μm; Mobile Phase A: water (0.1% FA), Mobile Phase B: MeCN; Flow rate: 60 mL/min; Gradient: 6% B to 36% B in 10 min; Wave Length: 254 nm) to afford the title compound (AGN-3) (3.3 mg, 6.8 μmol, 18%, 99.3% Purity) as a white solid. m/z 485.1 (M+H)⁺ (ES+). ¹H NMR (400 MHz, DMSO-d6) δ 8.50 (s, 1H), 7.89-7.82 (m, 1H), 7.77 (d, J=1.6 Hz, 1H), 7.68-7.56 (m, 3H), 7.42-7.35 (m, 1H), 7.14 (d, J=1.6 Hz, 1H), 3.30 (s, 3H), 2.26-2.14 (m, 1H), 1.11-0.89 (m, 8H).

### Example 446: 1-(2-{6-Cyclopropyl-4-[4-fluoro-2-(4-methyl-1,2,4-triazol-3-yl)phenyl]pyridin-2-yl}-7-fluoro-1,3-benzoxazol-5-yl)-N-[(1-methoxycyclobutyl) methyl]cyclopropan-1-amine (AGO-2)

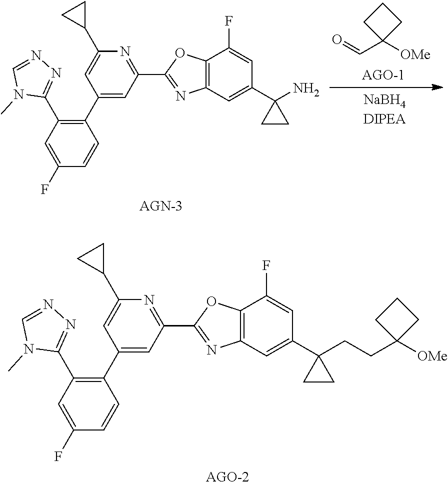 [SMILES: Cn1cnnc1-c1cc(F)ccc1-c1cc(-c2nc3cc(C4(N)CC4)cc(F)c3o2)nc(C2CC2)c1 ; COC1(C=O)CCC1 ; "AGN-3" ; COC1(CCC2(c3cc(F)c4oc(-c5cc(-c6ccc(F)cc6-c6nncn6C)cc(C6CC6)n5)nc4c3)CC2)CCC1 ; "AGO-2"]

To a stirred solution of compound (AGN-3) (40 mg, 1 Eq, 83 μmol) and 1-methoxycyclobutane-1-carbaldehyde (AGO-1) (19 mg, 2 Eq, 0.17 mmol) in MeOH (10 mL) was added DIPEA (16 mg, 1.5 Eq, 0.12 mmol) at rt. The resulting mixture was stirred for 12 h at 60° C. The mixture was allowed to cool down to rt. To the above mixture was added NaBH₄ (7 mg, 2 Eq, 0.17 mmol) at 0° C. The resulting mixture was stirred for additional 1 h at rt. The reaction was then quenched by the addition of 2 mL of MeOH at 0° C. The resulting mixture was concentrated in vacuo. The residue was purified by Prep-TLC with DCM/MeOH (8/1). The crude product was purified by Prep-HPLC with the following conditions (Column: XBridge Prep OBD C18 Column, 30*150 mm, 5 μm; Mobile Phase A: water (0.1% NH₄HCO₃+0.1% NH₃·H₂O), Mobile Phase B: MeCN; Flow rate: 60 mL/min; Gradient: 50% B to 60% B in 9 min; Wave Length: 254/220 nm) to afford the title compound (AGO-2) (7.0 mg, 12 μmol, 14%, 99.1% Purity) as a white solid. m/z 583.0 (M+H)⁺ (ES+). ¹H NMR (400 MHz, DMSO-d6) δ 8.50 (s, 1H), 7.88-7.84 (m, 1H), 7.79 (d, J=1.5 Hz, 1H), 7.66 (d, J=1.3 Hz, 1H), 7.65-7.59 (m, 2H), 7.52-7.48 (m, 1H), 7.11 (d, J=1.6 Hz, 1H), 3.29 (s, 3H), 2.97 (s, 3H), 2.57 (s, 2H), 2.22-2.15 (m, 1H), 2.02-1.93 (m, 2H), 1.86-1.80 (m, 2H), 1.69-1.61 (m, 1H), 1.52-1.42 (m, 1H), 1.10-0.97 (m, 6H), 0.95-0.90 (m, 2H).

### Example 447: (2-{6-Cyclopropyl-4-[4-fluoro-2-(4-methyl-1,2,4-triazol-3-yl)phenyl]pyridin-2-yl}-6-fluoro-7-methyl-1,3-benzoxazol-5-yl)methanol (AGP-7)

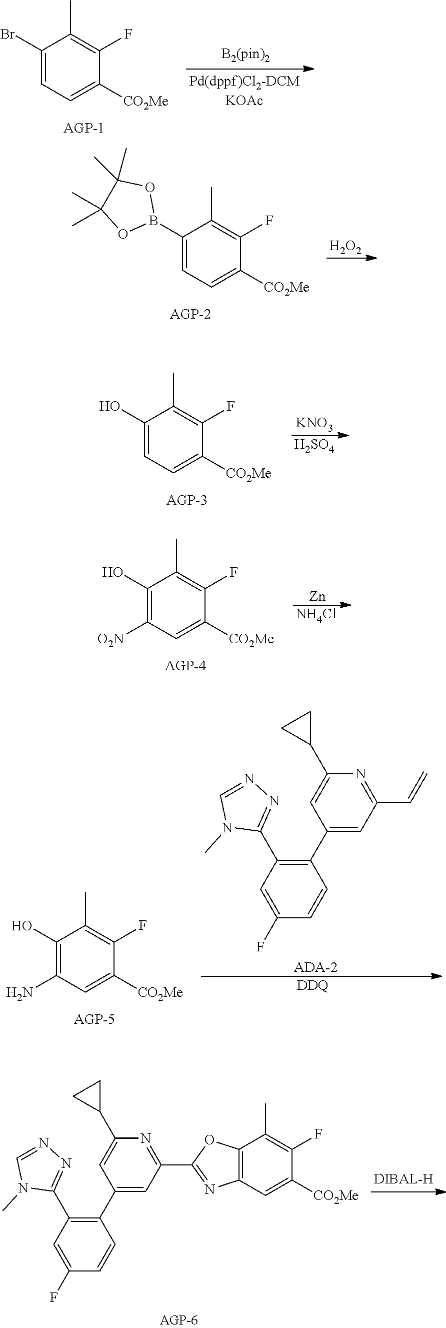

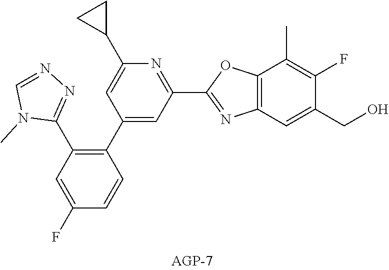 [SMILES: Cc1c(F)c(CO)cc2nc(-c3cc(-c4ccc(F)cc4-c4nncn4C)cc(C4CC4)n3)oc12]

Step 1: Methyl 2-fluoro-3-methyl-4-(4,4,5,5-tetramethyl-1,3,2-dioxaborolan-2-yl)benzoate (AGP-2). To a solution of methyl 4-bromo-2-fluoro-3-methylbenzoate (AGP-1) (1.00 g, 1 Eq, 4.05 mmol), bis(pinacolato)diboron (1.54 g, 1.5 Eq, 6.07 mmol) and KOAc (1.19 g, 3 Eq, 12.1 mmol) in 1,4-dioxane (20 mL) was added Pd(dppf)Cl₂·DCM (296 mg, 0.1 Eq, 0.41 mmol) at rt under nitrogen atmosphere. The resulting mixture was stirred for overnight at 80° C. under nitrogen atmosphere. The mixture was cooled to rt and concentrated in vacuo. The crude product was used in the next step directly without further purification. m/z 295.1 (M+H)⁺ (ES+).

Step 2: Methyl 2-fluoro-4-hydroxy-3-methylbenzoate (AGP-3). To a stirred mixture of the product from step 1 above (AGP-2) (1.20 g, 1 Eq, 4.08 mmol) in 1,4-dioxane (20 mL) was added aq. of H₂O₂ (1.85 g, 30% Wt, 4 Eq, 16.3 mmol) dropwise at 0° C. The resulting mixture was stirred for overnight at rt. The resulting mixture was concentrated in vacuo. The crude product was purified by reverse flash column Chromatography with the following conditions: Column, C18; mobile phase, water (0.1% FA) and MeCN (0% MeCN up to 80% in 20 min); Detector, UV 254/220 nm. This resulted in the sub-title compound (AGP-3) (500 mg, 2.72 mmol, 67%, 95% Purity) as a brown solid. m/z 185.1 (M+H)⁺ (ES+).

Step 3: Methyl 2-fluoro-4-hydroxy-3-methyl-5-nitrobenzoate (AGP-4). To a stirred solution of the product from step 2 above (AGP-3) (1.00 g, 1 Eq, 5.43 mmol) in conc. H₂SO₄ (20 mL) was added KNO₃ (1.65 g, 3 Eq, 16.3 mmol) in portions at 0° C. The resulting mixture was stirred overnight at rt. The resulting mixture was diluted with water and extracted with EtOAc (3×100 mL). The combined organic layers were washed with brine (2×100 mL), dried (Na₂SO₄). After filtration, the filtrate was concentrated in vacuo to afford the sub-title compound (AGP-4) (800 mg, 3.49 mmol, 64%, 75% Purity) as a brown solid. m/z 230.0 (M+H)⁺ (ES+).

Step 4: Methyl 5-amino-2-fluoro-4-hydroxy-3-methylbenzoate (AGP-5). To a stirred solution of the product from step 3 above (AGP-4) (400 mg, 1 Eq, 1.75 mmol) in THF (15 mL), MeOH (7.5 mL) and water (4.5 mL) were added Zn (456 mg, 4 Eq, 6.98 mmol) and NH₄Cl (934 mg, 10 Eq, 17.5 mmol) at rt. The resulting mixture was stirred for overnight at rt. The PH of mixture was acidified to 4 with aq. of HCl (1 M) at 0° C. The resulting mixture was concentrated in vacuo. The crude product was purified by reverse flash column Chromatography with the following conditions: Column, C18; mobile phase, water (0.1% FA) and MeCN (10% MeCN up to 80% in 20 min); Detector, UV 254/220 nm. The product-containing fractions were combined and evaporated to afford the sub-title compound (AGP-5) (100 mg, 0.50 mmol, 29%, 92% Purity) as a brown solid. m/z 200.1 (M+H)⁺ (ES+).

Step 5: Methyl 2-{6-cyclopropyl-4-[4-fluoro-2-(4-methyl-1,2,4-triazol-3-yl)phenyl]pyridin-2-yl}-6-fluoro-7-methyl-1,3-benzoxazole-5-carboxylate (AGP-6). A solution of intermediate (ADA-3) (70 mg, 1 Eq, 0.21 mmol) and the product from step 4 above (AGP-5) (87 mg, 2 Eq, 0.43 mmol) in DCM (15 mL) was stirred for 1 h at 60° C. The mixture was allowed to cool down to rt. To the above mixture was added DDQ (99 mg, 2 Eq, 0.43 mmol) at rt. The resulting mixture was stirred for 2 h at rt. The resulting mixture was diluted with water and extracted with EtOAc (3×20 mL). The combined organic layers were washed with brine (2×20 mL), dried (Na₂SO₄). After filtration, the filtrate was concentrated in vacuo. The crude product was purified by reverse flash column Chromatography with the following conditions: Column, C18; mobile phase, water (0.1% FA) and MeCN (35% MeCN up to 60% in 20 min); Detector, UV 254/220 nm. The product-containing fractions were combined and concentrated in vacuo. This resulted in the sub-title compound (AGP-6) (45 mg, 0.09 mmol, 41%, 93% Purity) as a white solid. m/z 502.2 (M+H)⁺ (ES+).

Step 6: (2-{6-Cyclopropyl-4-[4-fluoro-2-(4-methyl-1,2,4-triazol-3-yl)phenyl]pyridin-2-yl}-6-fluoro-7-methyl-1,3-benzoxazol-5-yl)methanol (AGP-7). To a stirred solution of the product from step 5 above (AGP-6) (40 mg, 1 Eq, 0.08 mmol) in THF (5 mL) was added DIBAL-H (g) in THF (0.64 mL, 1 M, 8 Eq, 0.64 mmol) was stirred for 2 h at 0° C. under nitrogen atmosphere. The reaction was then quenched by the addition of 3 mL of ice water at 0° C. The resulting mixture was diluted with water and extracted with EtOAc (3×20 mL). The combined organic layers were washed with brine (2×20 mL), dried (Na₂SO₄). After filtration, the filtrate was concentrated in vacuo. The residue was purified by Prep-TLC with DCM/MeOH (10/1). The crude product was purified by Prep-HPLC with the following conditions: Column: XBridge Prep OBD C18 Column, 30×150 mm, 5 μm; Mobile Phase A: water (0.1% FA) Mobile Phase B: MeCN; Flow rate: 60 mL/min; Gradient: 23 B to 53 B in 10 min; Detector, UV 254/210 nm; RT: 9.13. The product-containing fractions were combined and evaporated partially in vacuo and lyophilized overnight to afford the title compound (AGP-7) (10.0 mg, 21 μmol, 18%, 99.7% Purity) as a white solid. m/z 474.1 (M+H)⁺ (ES+). ¹H NMR (300 MHz, DMSO-d6) δ 8.51 (s, 1H), 7.92-7.81 (m, 1H), 7.74 (d, J=1.5 Hz, 1H), 7.72-7.56 (m, 3H), 7.09 (d, J=1.6 Hz, 1H), 5.39 (t, J=5.7 Hz, 1H), 4.64 (d, J=5.1 Hz, 2H), 3.29 (s, 3H), 2.47 (d, J=1.6 Hz, 3H), 2.24-2.09 (m, 1H), 1.10-0.97 (m, 2H), 0.97-0.87 (m, 2H).

### Example 448: (1S,2R)-2-{[(2-{6-Cyclopropyl-4-[4-fluoro-2-(4-methyl-1,2,4-triazol-3-yl)phenyl]pyridin-2-yl}-6-fluoro-7-methyl-1,3-benzoxazol-5-yl)methyl]amino}cyclopentan-1-ol (AGQ-2)

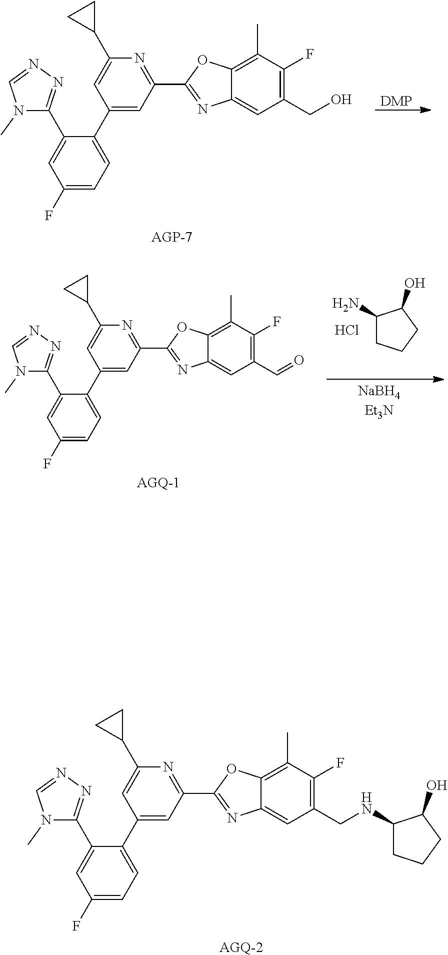

Step 1: 2-{6-Cyclopropyl-4-[4-fluoro-2-(4-methyl-1,2,4-triazol-3-yl)phenyl]pyridin-2-yl}-6-fluoro-7-methyl-1,3-benzoxazole-5-carbaldehyde (AGQ-1). To a stirred solution of compound (AGP-7) (25 mg, 1 Eq, 53 μmol) in DCM (5 mL) was added DMP (29 mg, 1.3 Eq, 69 μmol) at rt under air atmosphere. The resulting mixture was stirred for 1 h at rt. The resulting mixture was filtered; the filter cake was washed with DCM (3×3 mL). The filtrate was concentrated in vacuo. This resulted in the sub-title compound (AGQ-1) (20 mg, 42 μmol, 80%, 95% Purity) as a yellow oil. m/z 472.2 (M+H)⁺ (ES+)

Step 2: (1S,2R)-2-{[(2-{6-Cyclopropyl-4-[4-fluoro-2-(4-methyl-1,2,4-triazol-3-yl)phenyl]pyridin-2-yl}-6-fluoro-7-methyl-1,3-benzoxazol-5-yl)methyl]amino}cyclopentan-1-ol (AGQ-2). A solution of the product from step 1 above (AGQ-1) (20 mg, 1 Eq, 42 μmol) and (1S,2R)-2-aminocyclopentan-1-ol (R-2) (9 mg, 2 Eq, 84 μmol) in MeOH (3 mL) was added Et₃N (33 mg, 5 Eq, 0.33 mmol) for 2 h at 60° C. under nitrogen atmosphere. The mixture was allowed to cool down to rt. To the above mixture was added NaBH₄ (16 mg, 10 Eq, 0.42 mmol) at 0° C. The resulting mixture was stirred for 2 h at rt. The reaction was then quenched by the addition of 2 mL of MeOH at 0° C. The resulting mixture was diluted with water and extracted with DCM (3×10 mL). The combined organic layers were washed with brine (2×10 mL), dried (Na₂SO₄). After filtration, the filtrate was concentrated in vacuo. The crude product was purified by Prep-HPLC with the following conditions: Column: XBridge Prep OBD C18 Column, 30×150 mm, 5 μm; Mobile Phase A: water (0.1% NHLHCO₃+0.1% NH₃·H₂O) Mobile Phase B: MeCN; Flow rate: 60 mL/min; Gradient: 36 B to 46 B in 8 min; Detector, UV 254/210 nm; RT: 7.9. The product-containing fractions were combined and evaporated partially in vacuo and lyophilized overnight to afford the title compound (AGQ-2) (7.0 mg, 13 μmol, 30%, 99.5% Purity) as a white solid. m/z 557.3 (M+H)⁺ (ES+). ¹H NMR (400 MHz, Methanol-d4) δ 8.46 (s, 1H), 7.89-7.75 (m, 3H), 7.63-7.49 (m, 2H), 7.19 (d, J=1.6 Hz, 1H), 4.56-4.30 (m, 3H), 3.61-3.43 (m, 1H), 3.31 (d, J=1.6 Hz, 3H), 2.59 (d, J=1.6 Hz, 3H), 2.30-2.05 (m, 2H), 2.05-1.79 (m, 4H), 1.77-1.60 (m, 1H), 1.22-0.93 (m, 4H).

### Example 449: 2-(2-{6-Cyclopropyl-4-[4-fluoro-2-(4-methyl-1,2,4-triazol-3-yl)phenyl]pyridin-2-yl}-7-methyl-1,3-benzoxazol-5-yl)propan-2-amine (AGR-9)

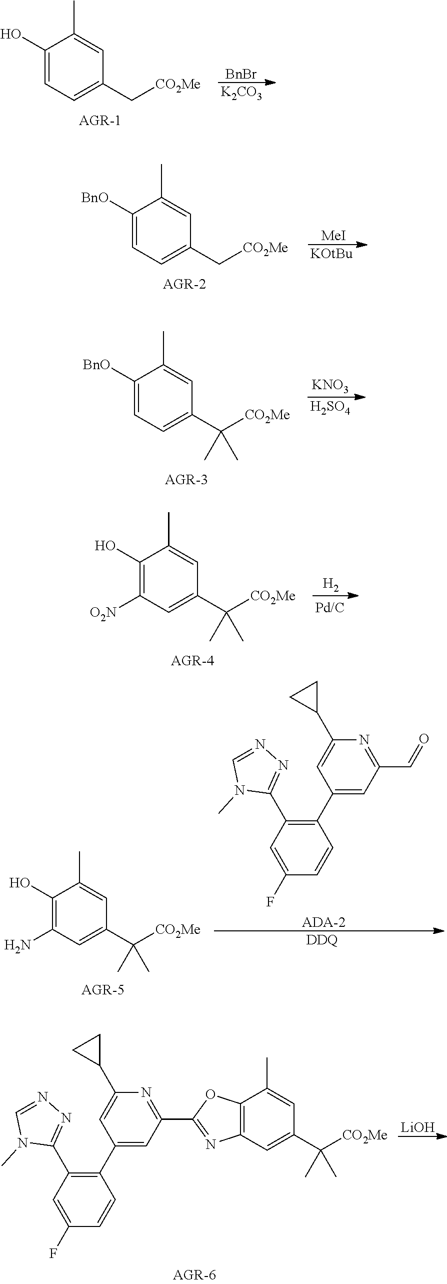

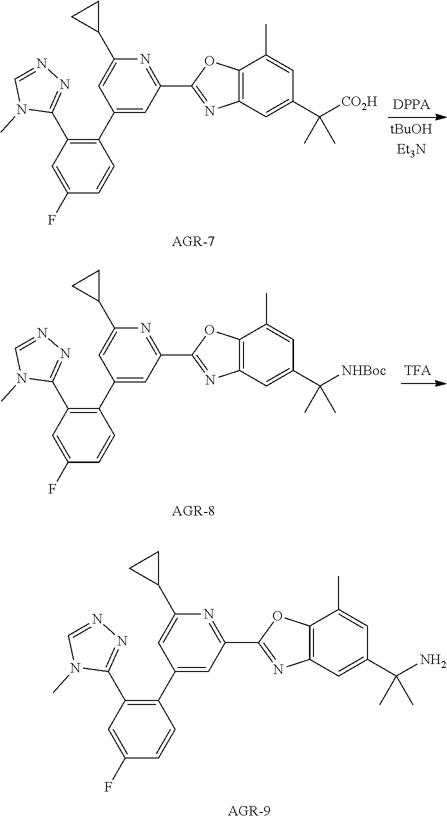 [SMILES: Cc1cc(C(C)(C)C(=O)O)cc2nc(-c3cc(-c4ccc(F)cc4-c4nncn4C)cc(C4CC4)n3)oc12 ; Cc1cc(C(C)(C)NC(=O)OC(C)(C)C)cc2nc(-c3cc(-c4ccc(F)cc4-c4nncn4C)cc(C4CC4)n3)oc12 ; Cc1cc(C(C)(C)N)cc2nc(-c3cc(-c4ccc(F)cc4-c4nncn4C)cc(C4CC4)n3)oc12]

Step 1: Methyl 2-[4-(benzyloxy)-3-methylphenyl]acetate (AGR-2). To a stirred solution of methyl 2-(4-hydroxy-3-methylphenyl)acetate (AGR-1) (2.00 g, 1 Eq, 11.1 mmol) in DMF (15 mL) were added K₂CO₃ (3.07 g, 2 Eq, 22.2 mmol) and benzyl bromide (2.85 g, 1.5 Eq, 16.6 mmol) at rt. The resulting mixture was stirred for 2 h at 60° C. The mixture was allowed to cool down to rt. The resulting mixture was diluted with water and extracted with EtOAc (3×200 mL). The combined organic layers were washed with brine (2×200 mL), dried (Na₂SO₄). After filtration, the filtrate was concentrated in vacuo. The residue was applied on a silica gel column chromatography with petroleum ether/EtOAc (5/1) to afford the sub-title compound (AGR-2) (2.8 g, 10.4 mmol, 93%, 90% Purity) as a colourless oil. m/z 271.1 (M+H)⁺ (ES+).

Step 2: Methyl 2-[4-(benzyloxy)-3-methylphenyl]-2-methylpropanoate (AGR-3). To a stirred solution of the product from step 1 above (AGR-2) (2.8 g, 1 Eq, 10.4 mmol) in THF (20 mL) were added t-BuOk (5.8 g, 5 Eq, 51.8 mmol) and CH₃I (7.35 g, 5 Eq, 51.8 mmol) at 0° C. The resulting mixture was stirred for 15 minutes at 0° C. The reaction was then quenched by the addition of 10 mL of MeOH at 0° C. The resulting mixture was concentrated in vacuo. The residue was purified by Prep-TLC with EtOAc/petroleum ether (1/6) to afford the sub-title compound (AGR-3) (1.86 g, 6.24 mmol, 60%, 92% Purity) as a colourless oil. m/z 299.2 (M+H)⁺ (ES+).

Step 3: Methyl 2-(4-hydroxy-3-methyl-5-nitrophenyl)-2-methylpropanoate (AGR-4). To a stirred solution of the product from step 2 above (AGR-3) (1.8 g, 1 Eq, 6.03 mmol) in conc. H₂SO₄ (15 mL) was added KNO₃ (671 mg, 1.1 Eq, 6.64 mmol) in portions at 0° C. The resulting mixture was stirred for 15 minutes at 0° C. The reaction was then quenched by the addition of 10 mL of ice water at 0° C. The resulting mixture was diluted with water and extracted with EtOAc (3×100 mL). The combined organic layers were washed with brine (2×100 mL), dried (Na₂SO₄). After filtration, the filtrate was concentrated in vacuo. The residue was purified by Prep-TLC with EtOAc/petroleum ether (1/3) to afford the sub-title compound (AGR-4) (210 mg, 0.83 mmol, 14%, 90% Purity) as a yellow solid. m/z 254.1 (M+H)⁺ (ES+).

Step 4: Methyl 2-(3-amino-4-hydroxy-5-methylphenyl)-2-methylpropanoate (AGR-5). To a solution of the product from step 3 above (AGR-4) (210 mg, 1 Eq, 0.83 mmol) in MeOH (15 mL) was added Pd/C (87.9 mg, 10% Wt, 10 Eq, 8.29 mmol) at rt under nitrogen atmosphere. The mixture was stirred for 2 h at rt under hydrogen atmosphere. The resulting mixture was filtered the filter cake was washed with MeOH (3×5 mL). The filtrate was concentrated in vacuo. The crude product was used in the next step directly without further purification. m/z 224.1 (M+H)⁺ (ES+).

Step 5: Methyl 2-(2-{6-cyclopropyl-4-[4-fluoro-2-(4-methyl-1,2,4-triazol-3-yl)phenyl]pyridin-2-yl}-7-methyl-1,3-benzoxazol-5-yl)-2-methylpropanoate (AGR-6). A solution of the product from step 4 above (AGR-5) (173 mg, 1 Eq, 0.78 mmol) and intermediate (ADA-2) (250 mg, 1 Eq, 0.78 mmol) in DCM (10 mL) was stirred for 1 h at 60° C. The mixture was allowed to cool down to rt. To the above mixture was added DDQ (352 mg, 1.55 mmol, 2 Eq) at rt. The resulting mixture was stirred for additional 1 h at rt. The resulting mixture was filtered; the filter cake was washed with DCM (3×5 mL). The filtrate was concentrated in vacuo. The residue was purified by TLC with DCM/MeOH (10/1) to afford the sub-title compound (AGR-6) (207 mg, 0.39 mmol, 51%, 92% Purity) as a black solid. m/z 526.2 (M+H)⁺ (ES+).

Step 6: 1-(2-{6-Cyclopropyl-4-[4-fluoro-2-(4-methyl-1,2,4-triazol-3-yl)phenyl]pyridin-2-yl}-7-fluoro-1,3-benzoxazol-5-yl)cyclopropane-1-carboxylic acid (AGR-7). To a stirred solution of the product from step 5 above (AGR-6) (207 mg, 1 Eq, 0.39 mmol) in THF (10 mL) and H₂O (2 mL) was added LiOH (47 mg, 5 Eq, 1.97 mmol) at rt. The resulting mixture was stirred for 12 h at 80° C. The mixture was allowed to cool down to rt and concentrated in vacuo. The pH value of the solution was adjusted to 3 with aq. of HCl (1 M) at 0° C. The resulting mixture was diluted with water and extracted with EtOAc (3×50 mL). The combined organic layers were washed with brine (2×50 mL), dried (Na₂SO₄). After filtration, the filtrate was concentrated in vacuo. The crude product was used in the next step directly without further purification. m/z 512.2 (M+H)⁺ (ES+).

Step 7: tert-Butyl N-[1-(2-{6-cyclopropyl-4-[4-fluoro-2-(4-methyl-1,2,4-triazol-3-yl)phenyl]pyridin-2-yl}-7-fluoro-1,3-benzoxazol-5-yl)cyclopropyl]carbamate (AGR-8). To a stirred solution of the product from step 6 above (AGR-7) (95 mg, 1 Eq, 0.19 mmol) in t-BuOH (15 mL) were added Et₃N (37 mg, 2 Eq, 0.37 mmol) and DPPA (61 mg, 1.2 Eq, 0.22 mmol) at rt under nitrogen atmosphere. The resulting mixture was stirred for 12 h at 80° C. under nitrogen atmosphere. The mixture was allowed to cool down to rt. The resulting mixture was diluted with water and extracted with EtOAc (3×20 mL). The combined organic layers were washed with brine (2×20 mL), dried (Na₂SO₄). After filtration, the filtrate was concentrated in vacuo. The residue was purified by TLC with DCM/MeOH (15/1) to afford the sub-title compound (AGR-8) (15 mg, 26 μmol, 14%, 95% Purity) as a colourless oil. m/z 583.3 (M+H)⁺ (ES+).

Step 8: 2-(2-{6-Cyclopropyl-4-[4-fluoro-2-(4-methyl-1,2,4-triazol-3-yl)phenyl]pyridin-2-yl}-7-methyl-1,3-benzoxazol-5-yl)propan-2-amine (AGR-9). A solution of the product from step 7 above (AGR-8) (15 mg, 1 Eq, 26 μmol) in HCl in 1,4-dioxane (5 mL, 4 M) was stirred for 1 h at rt. The resulting mixture was concentrated in vacuo. The crude product was purified by Prep-HPLC with the following conditions (Column: XBridge Prep OBD C18 Column, 30*150 mm, 5 μm; Mobile Phase A: water (0.1% NH₄HCO₃+0.1% NH₃·H₂O), Mobile Phase B: MeCN; Flow rate: 60 mL/min; Gradient: 30% B to 40% B in 9 min; Wave Length: 254/220 nm) to afford the title compound (AGR-9) (3.7 mg, 7.7 μmol, 30%, 99.3% Purity) as a white solid. m/z 483.1 (M+H)⁺ (ES+). ¹H NMR (400 MHz, DMSO-d6) δ 8.51 (s, 1H), 7.89-7.84 (m, 1H), 7.76 (d, J=1.8 Hz, 1H), 7.72 (d, J=1.5 Hz, 1H), 7.69-7.57 (m, 2H), 7.52-7.49 (m, 1H), 7.09 (d, J=1.6 Hz, 1H), 3.30 (s, 3H), 2.55 (s, 3H), 2.23-2.15 (m, 1H), 1.43 (s, 6H), 1.08-1.02 (m, 2H), 0.97-0.90 (m, 2H).

### Example 450: ({2-[4′-Fluoro-2′-(4-methyl-1,2,4-triazol-3-yl)-[1,1′-biphenyl]-3-yl]-7-methyl-1,3-benzoxazol-5-yl}methyl)[(2S)-1-methoxypropan-2-yl]amine (AGS-4)

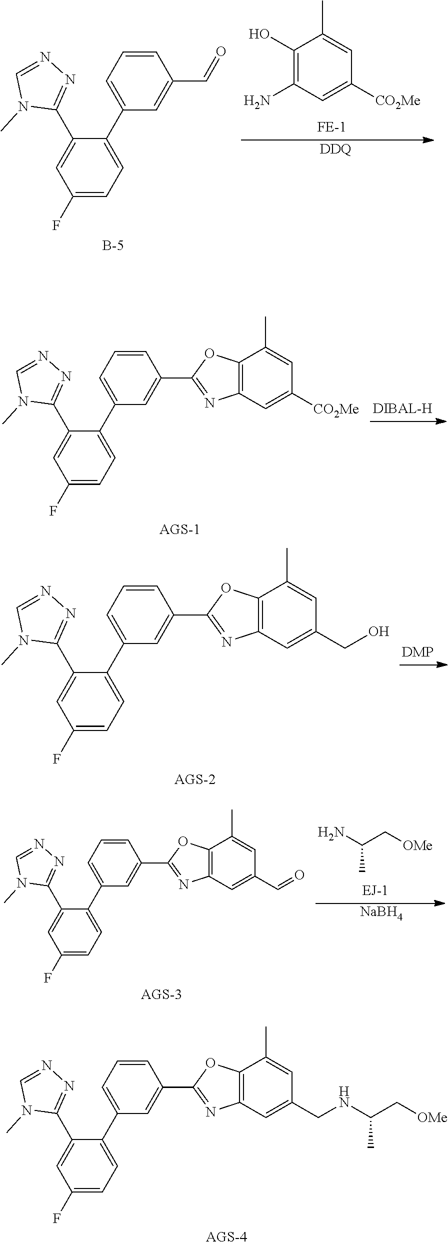

Step 1: Methyl 2-[4′-fluoro-2′-(4-methyl-1,2,4-triazol-3-yl)-[1,1′-biphenyl]-3-yl]-7-methyl-1,3-benzoxazole-5-carboxylate (AGS-1). A solution of intermediate (B-5) (30 mg, 1 Eq, 0.11 mmol) and intermediate (FE-1) (29 mg, 1.5 Eq, 0.16 mmol) in DCM (10 mL) was stirred for 1 h at 60° C. The mixture was allowed to cool down to rt. To the above mixture was added DDQ (48 mg, 2 Eq, 0.21 mmol) at rt. The resulting mixture was stirred for additional 2 h at rt. The resulting mixture was diluted with water and extracted with DCM (3×10 mL). The combined organic layers were washed with brine (2×10 mL), dried (Na₂SO₄). After filtration, the filtrate was concentrated in vacuo. The residue was purified by Prep-TLC with DCM/MeOH (10/1) to afford the sub-title compound (AGS-1) (20 mg, 45 μmol, 42%, 95% Purity) as a black solid. m/z 442.5 (M+H)⁺ (ES+).

Step 2: {2-[4′-Fluoro-2′-(4-methyl-1,2,4-triazol-3-yl)-[1,1′-biphenyl]-3-yl]-7-methyl-1,3-benzoxazol-5-yl}methanol (AGS-2). A solution of the product from step 1 above (AGS-1) (20 mg, 1 Eq, 45 μmol) and DIBAL-H (g) in THF (0.09 mL, 1 M, 2 Eq, 90 μmol) in THF (13 mL) was stirred for 1 h at rt. The reaction was then quenched by the addition of 2 mL of ice water at 0° C. The resulting mixture was concentrated in vacuo. The crude product was purified by Prep-HPLC with the following conditions (Column: XBridge Prep OBD C18 Column, 30*150 mm, 5 μm; Mobile Phase A: water (0.1% NH₄HCO₃+0.1% NH₃·H₂O), Mobile Phase B: MeCN; Flow rate: 60 mL/min; Gradient: 30% B to 40% B in 8 min; Wave Length: 254/220 nm; RT: 7.63) to afford the sub-title compound (AGS-2) (7.2 mg, 17 μmol, 38%, 99% Purity) as a white solid. m/z 415.2 (M+H)⁺ (ES+).

Step 3: 2-[4′-Fluoro-2′-(4-methyl-1,2,4-triazol-3-yl)-[1,1′-biphenyl]-3-yl]-7-methyl-1,3-benzoxazole-5-carbaldehyde (AGS-3). A solution of the product from step 2 above (AGS-2) (40 mg, 1 Eq, 97 μmol) and DMP (61.4 mg, 1.5 Eq, 0.15 mmol) in DCM (5 mL) was stirred for 1 h at rt. The resulting mixture was filtered; the filter cake was washed with DCM (3×5 mL). The filtrate was concentrated in vacuo. The crude product was used in the next step directly without further purification. m/z 412.4 (M+H)⁺ (ES+).

Step 4: ({2-[4′-Fluoro-2′-(4-methyl-1,2,4-triazol-3-yl)-[1,1′-biphenyl]-3-yl]-7-methyl-1,3-benzoxazol-5-yl}methyl)[(2S)-1-methoxypropan-2-yl]amine (AGS-4). A solution of the product from step 3 above (AGS-3) (40 mg, 1 Eq, 97 μmol) and (2S)-1-methoxypropan-2-amine (EJ-1) (13 mg, 1.5 Eq, 0.15 mmol) in MeOH (10 mL) was stirred for overnight at rt. To the above mixture was added NaBH₄ (7 mg, 2 Eq, 0.19 mmol) at 0° C. The resulting mixture was stirred for additional 1 h at rt. The reaction was then quenched by the addition of 2 mL of MeOH at 0° C. The resulting mixture was concentrated in vacuo. The crude product was purified by Prep-HPLC with the following conditions (Column: XBridge Prep OBD C18 Column, 30*150 mm, 5 μm; Mobile Phase A: water (0.1% NH₄HCO₃+0.1% NH₃·H₂O), Mobile Phase B: MeCN; Flow rate: 60 mL/min; Gradient: 38% B to 48% B in 8 min; Wave Length: 254/220 nm; RT: 6.95) to afford the title compound (AGS-4) (10.3 mg, 21 μmol, 22%, 99.5% Purity) as a white solid. m/z 486.0 (M+H)⁺ (ES+). ¹H NMR (300 MHz, DMSO-d6) δ 8.43 (d, J=1.4 Hz, 1H), 8.14 (d, J=7.8 Hz, 1H), 7.91 (d, J=1.9 Hz, 1H), 7.79 (m, 1H), 7.65-7.50 (m, 4H), 7.35 (d, J=7.8 Hz, 1H), 7.23 (s, 1H), 3.88-3.73 (m, 2H), 3.30-3.19 (m, 5H), 3.16 (s, 3H), 2.80-2.74 (m, 1H), 2.57 (s, 3H), 0.98 (d, J=6.3 Hz, 3H).

### Example 451: (Cyclobutylmethyl)[(2-{6-cyclopropyl-4-[4-fluoro-2-(4-methyl-1,2,4-triazol-3-yl)phenyl]pyridin-2-yl}-7-fluoro-1,3-benzoxazol-5-yl)methyl]amine (AGT-1)

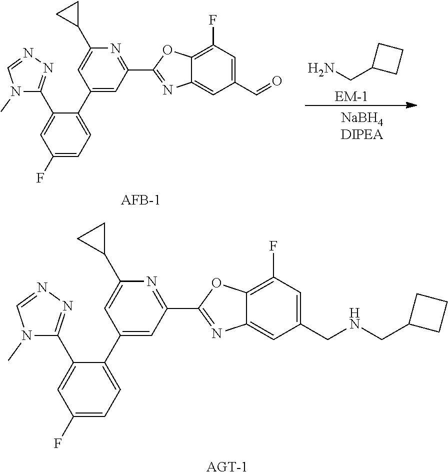

A solution of intermediate (AFB-1) (30 mg, 1 Eq, 66 μmol) and 1-cyclobutylmethanamine (EM-1) (8 mg, 1.5 Eq, 99 μmol) in MeOH (3 mL) was added DIPEA (42 mg, 5 Eq, 0.33 mmol) for 2 h at 60° C. under nitrogen atmosphere. The mixture was allowed to cool down to rt. To the above mixture was added NaBH₄ (16 mg, 10 Eq, 0.42 mmol) at 0° C. The resulting mixture was stirred for 2 h at rt. The reaction was then quenched by the addition of 2 mL of MeOH at 0° C. The resulting mixture was diluted with water and extracted with DCM (3×10 mL). The combined organic layers were washed with brine (2×10 mL), dried (Na₂SO₄). After filtration, the filtrate was concentrated in vacuo. The crude product was purified by Prep-HPLC with the following conditions: (Column: XBridge Prep OBD C18 Column, 30*150 mm, 5 μm; Mobile Phase A: water (0.1% NH₄HCO₃+0.1% NH₃·H₂O), Mobile Phase B: MeCN; Flow rate: 60 mL/min; Gradient: 45% B to 55% B in 8 min; Wave Length: 254 nm; RT: 7.9). The product-containing fractions were combined and evaporated partially in vacuo and lyophilized overnight to afford the title compound (AGT-1) (8.4 mg, 16 μmol, 24%, 99.6% Purity) as a white solid. m/z 527.7 (M+H)⁺ (ES+). ¹H NMR (400 MHz, Methanol-d4) δ 8.46 (s, 1H), 7.92 (d, J=1.6 Hz, 1H), 7.86-7.81 (m, 1H), 7.62-7.48 (m, 3H), 7.35-7.29 (m, 1H), 7.10 (d, J=1.5 Hz, 1H), 3.87 (s, 2H), 3.34 (s, 3H), 2.62 (d, J=7.7 Hz, 2H), 2.57-2.50 (m, 1H), 2.21-2.15 (m, 1H), 2.12-2.06 (m, 2H), 1.95-1.89 (m, 1H), 1.86-1.81 (m, 1H), 1.74-1.67 (m, 2H), 1.08-1.01 (m, 4H).

### Example 452: 1-(2-{6-Cyclopropyl-4-[4-fluoro-2-(4-methyl-1,2,4-triazol-3-yl)phenyl]pyridin-2-yl}-7-methyl-1,3-benzoxazol-5-yl)ethanone (AGU-1)

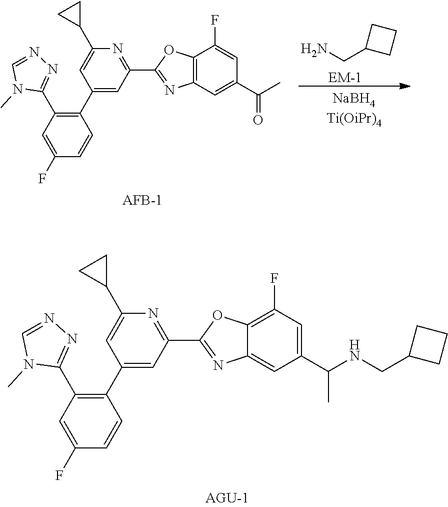

To a stirred solution of intermediate (AFQ-4) (150 mg, 1 Eq, 0.32 mmol) and 1-cyclobutylmethanamine (EM-1) (82 mg, 3 Eq, 0.96 mmol) in THF (8 mL) was added Ti(Oi-Pr)₄ (912 mg, 10 Eq, 3.21 mmol) at rt under nitrogen atmosphere. The resulting mixture was stirred for 2 h at rt under nitrogen atmosphere. To the above mixture was added NaBH₄ (24 mg, 2 Eq, 0.64 mmol) at 0° C. The resulting mixture was stirred for 2 h at rt. The reaction was then quenched by the addition of 2 mL of MeOH at 0° C. The resulting mixture was diluted with water and extracted with DCM (3×20 mL). The combined organic layers were washed with brine (2×20 mL), dried (Na₂SO₄). After filtration, the filtrate was concentrated in vacuo. The crude product was purified by Prep-HPLC with the following conditions: (Column: XBridge Prep OBD C18 Column, 30*150 mm, 5 μm; Mobile Phase A: water (0.1% NH₄HCO₃+0.1% NH₃·H₂O), Mobile Phase B: MeCN; Flow rate: 60 mL/min; Gradient: 46% B to 60% B in 9 min; Wave Length: 254/220 nm; RT: 8.25) to afford the title compound (AGU-1) (8.1 mg, 15 μmol, 79%, 98.0% Purity) as a white solid. m/z 537.8 (M+H)⁺ (ES+). ¹H NMR (400 MHz, Methanol-d4) δ 8.46 (s, 1H), 7.98-7.74 (m, 2H), 7.73-7.39 (m, 3H), 7.28 (t, J=1.3 Hz, 1H), 7.10 (d, J=1.6 Hz, 1H), 3.95-3.79 (m, 1H), 3.31 (s, 3H), 2.62 (s, 3H), 2.57-2.34 (m, 3H), 2.23-2.14 (m, 1H), 2.13-1.95 (m, 2H), 1.93-1.72 (m, 2H), 1.69-1.55 (m, 2H), 1.42 (d, J=6.6 Hz, 3H), 1.24-0.82 (m, 4H).

### Example 453: (cyclobutylmethyl)[(1S)-1-(2-{6-cyclopropyl-4-[4-fluoro-2-(4-methyl-1,2,4-triazol-3-yl)phenyl]pyridin-2-yl}-7-methyl-1,3-benzoxazol-5-yl)ethyl]amine (AGX-1)

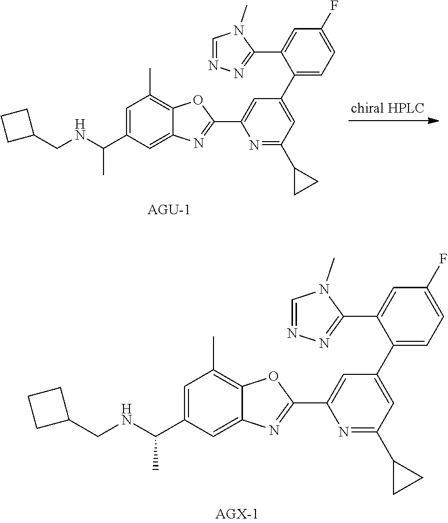 [SMILES: Cc1cc(C(C)NCC2CCC2)cc2nc(-c3cc(-c4ccc(F)cc4-c4nncn4C)cc(C4CC4)n3)oc12 ; Cc1cc([C@H](C)NCC2CCC2)cc2nc(-c3cc(-c4ccc(F)cc4-c4nncn4C)cc(C4CC4)n3)oc12]

The crude product of (cyclobutylmethyl)[1-(2-{6-cyclopropyl-4-[4-fluoro-2-(4-methyl-1,2,4-triazol-3-yl)phenyl]pyridin-2-yl}-7-methyl-1,3-benzoxazol-5-yl)ethyl]amine (AGU-1) (20 mg, 1 Eq, 37 μmol) was purified by Prep-Chiral-HPLC with the following conditions (Column: CHIRALPAK IG, 2*25 cm, 5 μm; Mobile Phase A: Hex:DCM=3:1 (0.5%2M NH₃-MeOH)-HPLC, Mobile Phase B: EtOH-HPLC; Flow rate: 20 mL/min; Gradient: 50% B to 50% B in 28 min; Wave Length: 220/254 nm; RT1(min): 18.727) to afford the title compound (AGX-1) (4.3 mg, 8.0 μmol, 21%, 96.0% Purity) as a yellow solid. m/z 537.4 (M+H)⁺ (ES+). ¹H NMR (400 MHz, Methanol-d4) δ 8.50 (s, 1H), 7.91-7.84 (m, 2H), 7.63-7.52 (m, 3H), 7.29-7.11 (m, 2H), 3.98-3.91 (m, 1H), 3.30 (s, 3H), 2.65 (s, 3H), 2.60-2.43 (m, 3H), 2.24-2.18 (m, 1H), 2.13-2.05 (m, 2H), 2.02-1.86 (m, 1H), 1.86-1.80 (m, 1H), 1.71-1.60 (m, 2H), 1.47 (d, J=6.6 Hz, 3H), 1.09 (d, J=5.5 Hz, 4H). Column: CHIRALPAK IH-3, 4.6*50 mm, 3 μm; Mobile Phase A: Hex:DCM=3:1) (0.1% DEA):EtOH=50:50, Flow rate: 1 mL/min; RT: 1.932.

### Example 454: (cyclobutylmethyl)[(1R)-1-(2-{6-cyclopropyl-4-[4-fluoro-2-(4-methyl-1,2,4-triazol-3-yl)phenyl]pyridin-2-yl}-7-methyl-1,3-benzoxazol-5-yl)ethyl]amine (AGY-1)

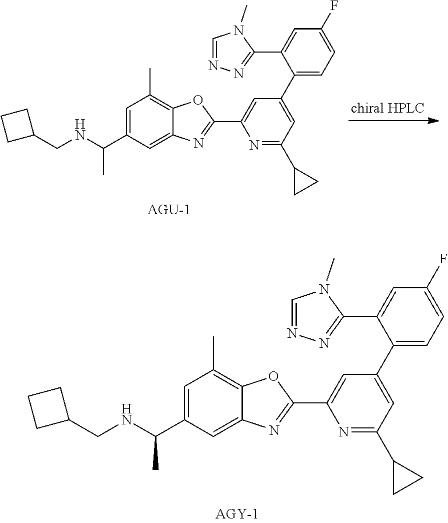 [SMILES: Cc1cc(C(C)NCC2CCC2)cc2nc(-c3cc(-c4ccc(F)cc4-c4nncn4C)cc(C4CC4)n3)oc12 ; Cc1cc([C@@H](C)NCC2CCC2)cc2nc(-c3cc(-c4ccc(F)cc4-c4nncn4C)cc(C4CC4)n3)oc12]

The crude product of (cyclobutylmethyl)[1-(2-{6-cyclopropyl-4-[4-fluoro-2-(4-methyl-1,2,4-triazol-3-yl)phenyl]pyridin-2-yl}-7-methyl-1,3-benzoxazol-5-yl)ethyl]amine (AGU-1) (20 mg, 1 Eq, 37 μmol) was purified by Prep-Chiral-HPLC with the following conditions (Column: CHIRALPAK IG, 2*25 cm, 5 μm; Mobile Phase A: Hex:DCM=3:1 (0.5%2M NH₃-MeOH)-HPLC, Mobile Phase B: EtOH-HPLC; Flow rate: 20 mL/min; Gradient: 50% B to 50% B in 28 min; Wave Length: 220/254 nm; RT2(min): 19.08) to afford the title compound (AGY-1) (4.1 mg, 7.6 μmol, 20%, 99.0% Purity) as a white solid. m/z 537.4 (M+H)⁺ (ES+). ¹H NMR (400 MHz, Methanol-d4) δ 8.50 (s, 1H), 7.91-7.84 (m, 2H), 7.64-7.52 (m, 3H), 7.29-7.11 (m, 2H), 3.96-3.89 (m, 1H), 3.30 (s, 3H), 2.65 (s, 3H), 2.59-2.42 (m, 3H), 2.24-2.18 (m, 1H), 2.14-2.04 (m, 2H), 1.95-1.78 (m, 2H), 1.70-1.59 (m, 2H), 1.46 (d, J=6.6 Hz, 3H), 1.09 (d, J=5.6 Hz, 4H). Column: CHIRALPAK IH-3, 4.6*50 mm, 3 μm; Mobile Phase A: Hex:DCM=3:1) (0.1% DEA):EtOH=50:50, Flow rate: 1 mL/min; RT: 2.799.

### Example 455: [(2-{6-cyclopropyl-4-[4-fluoro-2-(4-methyl-1,2,4-triazol-3-yl)phenyl]pyridin-2-yl}-6,7-difluoro-1,3-benzoxazol-5-yl)methyl](ethyl)amine (AGZ-2)

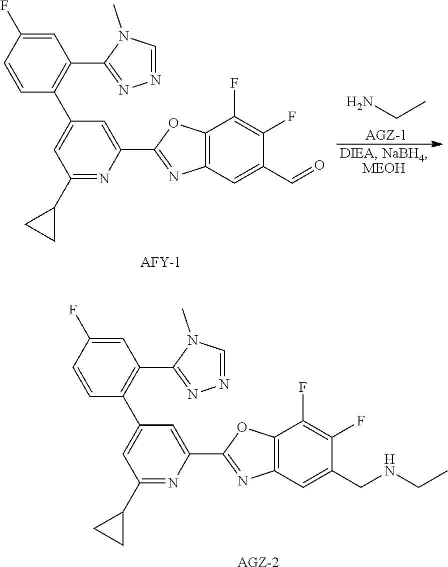

A solution of 2-{6-cyclopropyl-4-[4-fluoro-2-(4-methyl-1,2,4-triazol-3-yl)phenyl]pyridin-2-yl}-6,7-difluoro-1,3-benzoxazole-5-carbaldehyde (AFY-1) (24 mg, 1 Eq, 0.05 mmol) and ethylamine (AGZ-1) (3 mg, 1.3 Eq, 65 μmol) in MeOH (3 mL) was added DIPEA (33 mg, 5 Eq, 0.25 mmol) for 2 h at 60° C. under nitrogen atmosphere. The mixture was allowed to cool down to room temperature. To the above mixture was added NaBH₄ (10 mg, 5 Eq, 0.25 mmol) at 0° C. The resulting mixture was stirred for 2 h at room temperature. The reaction was then quenched by the addition of 2 mL of methyl alcohol at 0° C. The resulting mixture was diluted with water and extracted with dichloromethane (3×20 mL). The combined organic layers were washed with brine (2×20 mL), dried over anhydrous sodium sulfate. After filtration, the filtrate was concentrated under reduced pressure. The crude product was purified by Prep-HPLC with the following conditions: (Column: XBridge BEH C18 OBD Prep Column, 19*250 mm, 5 μm; Mobile Phase A: Water (0.1% NH₄HCO₃), Mobile Phase B: ACN; Flow rate: 25 mL/min; Gradient: 45% B to 52% B in 6 min; Wave Length: 254 nm) RT: 7.9. The product-containing fractions were combined and evaporated partially in vacuo and lyophilized overnight to afford the title compound (AGZ-2) (14.6 mg, 8.5 μmol, 56%, 97.1% Purity) as a white solid. m/z 505.3 (M+H)⁺ (ES+). ¹H NMR (400 MHz, Methanol-d4) δ 8.49 (s, 1H), 7.93 (s, 1H), 7.88-7.83 (m, 1H), 7.68 (d, J=5.5 Hz, 1H), 7.63-7.57 (m, 1H), 7.55-7.51 (m, 1H), 7.14 (s, 1H), 3.98 (s, 2H), 3.30 (s, 3H), 2.77-2.67 (m, 2H), 2.24-2.15 (m, 1H), 1.23-1.17 (m, 3H), 1.12-1.05 (m, 4H).

### Example 456: [(2-{6-cyclopropyl-4-[4-fluoro-2-(4-methyl-1,2,4-triazol-3-yl)phenyl]pyridin-2-yl}-6,7-difluoro-1,3-benzoxazol-5-yl)methyl](methyl)amine (AHA-2)

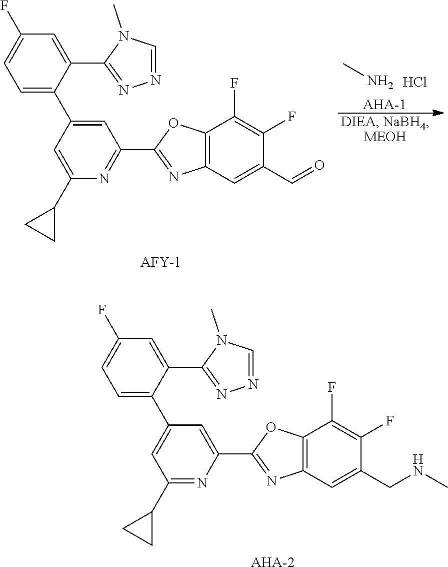

To a stirred mixture of 2-{6-cyclopropyl-4-[4-fluoro-2-(4-methyl-1,2,4-triazol-3-yl)phenyl]pyridin-2-yl}-6,7-difluoro-1,3-benzoxazole-5-carbaldehyde (AFY-1) (30 mg, 1 Eq, 63 μmol) and methanamine hydrochloride (AHA-1) (3 mg, 1.3 Eq, 82 μmol) in MeOH (3 mL) was added DIPEA (0.2 mL) for 2 h at 60° C. under nitrogen atmosphere. The mixture was allowed to cool down to room temperature. To the above mixture was added NaBH₄ (7 mg, 3 Eq, 0.19 mmol) at room temperature. The reaction was then quenched by the addition of 2 mL of methyl alcohol at 0° C. The resulting mixture was concentrated under reduced pressure. The crude product was purified by Prep-HPLC with the following conditions (Column: XBridge Prep OBD C18 Column, 19*250 mm, 5 μm; Mobile Phase A: Water (0.1% NH₄HCO₃), Mobile Phase B: ACN; Flow rate: 25 mL/min; Gradient: 35% B to 55% B in 5.8 min; Wave Length: 254/210 nm; RT: 5.5) to afford the title compound (AHA-2) (3.0 mg, 6.1 μmol, 9.6%, 99.3% Purity) as a white solid. m/z 491.2 (M+H)⁺ (ES+). ¹H NMR (400 MHz, Methanol-d4) δ 8.49 (s, 1H), 7.93 (s, 1H), 7.86 (s, 1H), 7.69 (d, J=5.2 Hz, 1H), 7.60 (s, 1H), 7.54 (d, J=8.7 Hz, 1H), 7.15 (s, 1H), 3.99 (s, 2H), 3.30 (s, 3H), 2.48 (s, 3H), 2.19 (s, 1H), 1.09 (d, J=8.2 Hz, 4H).

### Example 457: [(2-{6-cyclopropyl-4-[4-fluoro-2-(4-methyl-1,2,4-triazol-3-yl)phenyl]pyridin-2-yl}-7-fluoro-1,3-benzoxazol-5-yl)methyl](2-methoxyethyl)amine (AHB-1)

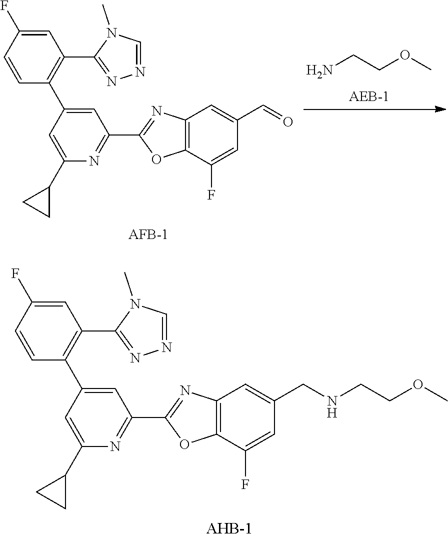

To a stirred mixture of 2-{6-cyclopropyl-4-[4-fluoro-2-(4-methyl-1,2,4-triazol-3-yl)phenyl]pyridin-2-yl}-7-fluoro-1,3-benzoxazole-5-carbaldehyde (AFB-1) (30 mg, 1 Eq, 66 μmol) and 2-methoxyethan-1-amine (AEB-1) (9 mg, 1.8 Eq, 0.12 mmol) in MeOH (8 mL) was added DIPEA (25 mg, 3 Eq, 0.20 mmol) for 2 h at 60° C. under nitrogen atmosphere. The mixture was allowed to cool down to room temperature. To the above mixture was added NaBH₄ (7 mg, 3 Eq, 0.20 mmol) at 0° C. The resulting mixture was stirred for overnight at room temperature. The reaction was then quenched by the addition of 2 mL of methyl alcohol at 0° C. The resulting mixture was concentrated under reduced pressure. The crude product was purified by Prep-HPLC with the following conditions (Column: XBridge Prep OBD C18 Column, 30*150 mm, 5 μm; Mobile Phase A: Water (0.1% NH₄HCO₃+0.1% NH₃·H₂O), Mobile Phase B: ACN; Flow rate: 60 mL/min; Gradient: 30% B to 55% B in 9 min; Wave Length: 254/220 nm; RT: 8.83) to afford the title compound (AHB-1) (10.2 mg, 20 μmol, 30%, 98.3% Purity) as a white solid. m/z 517.1 (M+H)⁺ (ES+). ¹H NMR (400 MHz, DMSO-d6) δ 8.50 (s, 1H), 7.90-7.80 (m, 2H), 7.68-7.58 (m, 3H), 7.40 (d, J=11.4, 1.3 Hz, 1H), 7.14-7.09 (m, 1H), 3.84 (s, 2H), 3.44-3.37 (m, 2H), 3.30 (s, 3H), 3.24 (s, 3H), 2.67-2.60 (m, 2H), 2.22-2.11 (m, 1H), 1.09-0.89 (m, 4H).

### Example 468: methyl 2-{4-[4-cyano-2-(4-methyl-1,2,4-triazol-3-yl) phenyl]-6-cyclopropylpyridin-2-yl}-7-fluoro-1,3-benzoxazole-5-carboxylate (AHC-1)

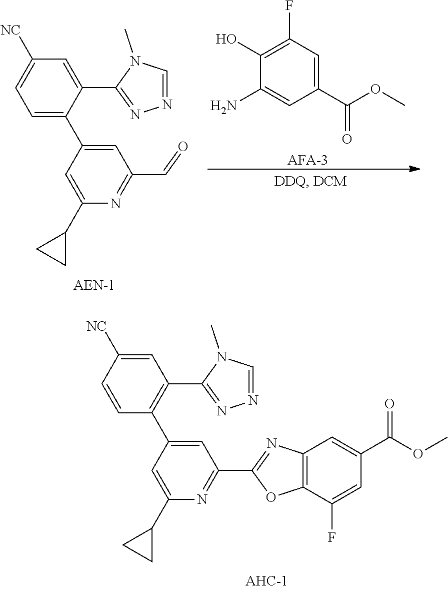 [SMILES: Cn1cnnc1-c1cc(C#N)ccc1-c1cc(C=O)nc(C2CC2)c1 ; COC(=O)c1cc(N)c(O)c(F)c1 ; "AFA-3" ; COC(=O)c1cc(F)c2oc(-c3cc(-c4ccc(C#N)cc4-c4nncn4C)cc(C4CC4)n3)nc2c1]

A solution of 4-(2-cyclopropyl-6-formylpyridin-4-yl)-3-(4-methyl-1,2,4-triazol-3-yl) benzonitrile (AEN-1) (40 mg, 1 Eq, 0.12 mmol) and methyl 3-amino-5-fluoro-4-hydroxybenzoate (AFA-1) (23 mg, 1 Eq, 0.12 mmol) in DCM (10 mL) was stirred for 1 h at room temperature. To the above mixture was added DDQ (56 mg, 2 Eq, 0.24 mmol) at room temperature. The resulting mixture was stirred for additional 1 h at room temperature. The resulting mixture was filtered; the filter cake was washed with DCM (3×3 mL). The filtrate was concentrated under reduced pressure. The residue was purified by TLC with dichloromethane/methanol (12/1). The crude product was purified by Prep-HPLC with the following conditions (Column: XBridge Shield RP18 OBD Column, 19*250 mm, 10 μm; Mobile Phase A: Water (0.1% NH₄HCO₃), Mobile Phase B: ACN; Flow rate: 25 mL/min; Gradient: 50% B to 70% B in 6 min; Wave Length: 254 nm)) to afford the title compound (AHC-1) (7.8 mg, 16 μmol, 13%, 99.6% Purity) as a white solid. m/z 495.2 (M+H)⁺ (ES+). ¹H NMR (400 MHz, DMSO-d6) δ 8.55 (s, 1H), 8.26 (d, J=4.3 Hz, 3H), 8.07-7.89 (m, 3H), 7.22 (s, 1H), 3.93 (s, 3H), 3.38 (s, 3H), 2.26-2.18 (m, 1H), 1.07 (d, J=8.0 Hz, 2H), 1.00-0.93 (m, 2H).

### Example 459: 4-{2-cyclopropyl-6-[7-fluoro-5-(hydroxymethyl)-1,3-benzoxazol-2-yl]pyridin-4-yl}-3-(4-methyl-1,2,4-triazol-3-yl) benzonitrile (AHD-1)

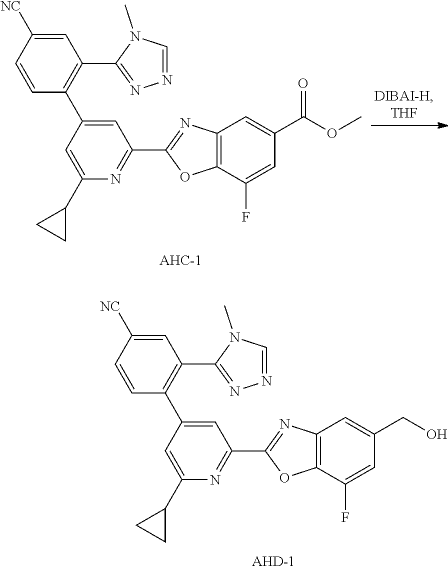 [SMILES: COC(=O)c1cc(F)c2oc(-c3cc(-c4ccc(C#N)cc4-c4nncn4C)cc(C4CC4)n3)nc2c1 ; Cn1cnnc1-c1cc(C#N)ccc1-c1cc(-c2nc3cc(CO)cc(F)c3o2)nc(C2CC2)c1 ; "AHD-1"]

To a stirred solution of methyl 2-{4-[4-cyano-2-(4-methyl-1,2,4-triazol-3-yl) phenyl]-6-cyclopropylpyridin-2-yl}-7-fluoro-1,3-benzoxazole-5-carboxylate (AHC-1) (100 mg, 1 Eq, 0.20 mmol) in THF (10 mL) was added DIBAl-H (g) in THF (0.65 mL, 2.5 M, 8 Eq, 1.62 mmol) in portions at 0° C. under nitrogen atmosphere. The resulting mixture was stirred for 15 minutes at 0° C. under nitrogen atmosphere. The reaction was then quenched by the addition of 2 mL of ice water at 0° C. The resulting mixture was concentrated under vacuum. The residue was purified by TLC with dichloromethane/methanol (10/1). The crude product was purified by Prep-HPLC with the following conditions (Column: XBridge Prep OBD C18 Column, 30*150 mm, 5 μm; Mobile Phase A: Water (0.1% NH₄HCO₃+0.1% NH₃·H₂O), Mobile Phase B: ACN; Flow rate: 60 mL/min; Gradient: 33% B to 43% B in 9 min; Wave Length: 254/220 nm) to afford the title compound (AHD-1) (3.4 mg, 7.3 μmol, 3.5%, 97.5% Purity) as a white solid. m/z 467.2 (M+H)⁺ (ES+). ¹H NMR (400 MHz, DMSO-d6) δ 8.55 (d, J=2.7 Hz, 1H), 8.28-8.24 (m, 2H), 8.05-7.99 (m, 1H), 7.88 (d, J=1.8 Hz, 1H), 7.64 (s, 1H), 7.37 (d, J=11.2 Hz, 1H), 7.19 (d, J=1.9 Hz, 1H), 5.50 (t, J=5.9 Hz, 1H), 4.64 (d, J=5.5 Hz, 2H), 3.37 (d, J=2.6 Hz, 3H), 2.26-2.15 (m, 1H), 1.09-1.05 (m, 2H), 1.05-0.95 (m, 2H).

### Example 460: 4-[2-(5-{1[(cyclobutylmethyl)amino]methyl}-7-fluoro-1,3-benzoxazol-2-yl)-6-cyclopropylpyridin-4-yl]-3-(4-methyl-1,2,4-triazol-3-yl) benzonitrile (AHE-2)

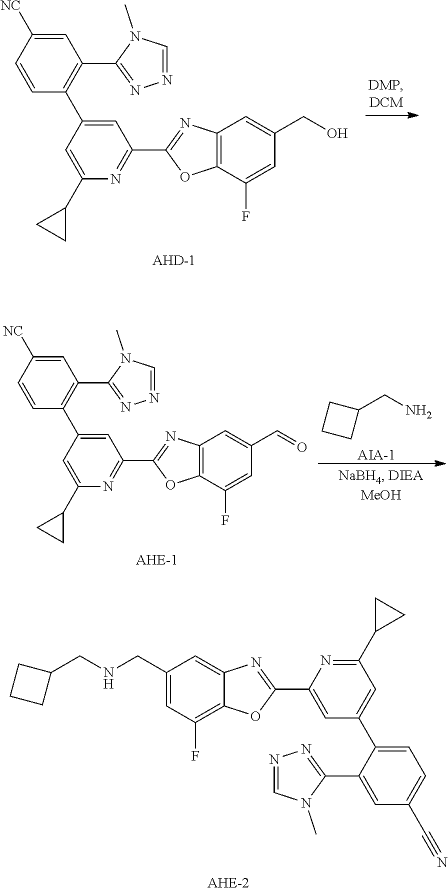

Step 1: 4-[2-cyclopropyl-6-(7-fluoro-5-formyl-1,3-benzoxazol-2-yl) pyridin-4-yl]-3-(4-methyl-1,2,4-triazol-3-yl) benzonitrile (AHE-1). A solution of 4-{2-cyclopropyl-6-[7-fluoro-5-(hydroxymethyl)-1,3-benzoxazol-2-yl]pyridin-4-yl}-3-(4-methyl-1,2,4-triazol-3-yl) benzonitrile (AHD-1) (30 mg, 1 Eq, 64 μmol) and DMP (36 mg, 1.3 Eq, 83 μmol) in DCM (10 mL) was stirred for 1 h at room temperature. The resulting mixture was filtered; the filter cake was washed with DCM (3×3 mL). The filtrate was concentrated under reduced pressure. The crude product was used in the next step directly without further purification. m/z 465.1 (M+H)+(ES+).

Step 2: 4-[2-(5-{[(cyclobutylmethyl)amino]methyl}-7-fluoro-1,3-benzoxazol-2-yl)-6-cyclopropylpyridin-4-yl]-3-(4-methyl-1,2,4-triazol-3-yl) benzonitrile (AHE-2). To a stirred solution of the product from step 1 above (AHE-1) (25 mg, 1 Eq, 54 μmol) and 1-cyclobutylmethanamine (AIA-1) (7 mg, 1.5 Eq, 81 μmol) in MeOH (10 mL) was added DIPEA (9 mg, 1.2 Eq, 65 μmol) at room temperature. The resulting mixture was stirred for 12 h at 60° C. The mixture was allowed to cool down to room temperature. To the above mixture was added NaBH₄ (4 mg, 2 Eq, 0.11 mmol) at 0° C. The resulting mixture was stirred for additional 1 h at room temperature. The reaction was then quenched by the addition of 2 mL of methyl alcohol at 0° C. The resulting mixture was concentrated under vacuum. The residue was purified by TLC with dichloromethane/methanol (9/1). The crude product was purified by Prep-HPLC with the following conditions (Column: XBridge Prep OBD C18 Column, 30*150 mm, 5 μm; Mobile Phase A: Water (0.1% NH₄HCO₃+0.1% NH₃·H₂O), Mobile Phase B: ACN; Flow rate: 60 mL/min; Gradient: 43% B to 53% B in 9 min; Wave Length: 254/220 nm) to afford the title compound (AHE-2) (8.1 mg, 15 μmol, 28%, 98.3% Purity) as a white solid. m/z 534.2 (M+H)⁺ (ES+). ¹H NMR (400 MHz, DMSO-d6) δ 8.54 (s, 1H), 8.26 (d, J=6.7 Hz, 2H), 8.01 (d, J=8.4 Hz, 1H), 7.85 (s, 1H), 7.64 (s, 1H), 7.41 (d, J=11.4 Hz, 1H), 7.19 (s, 1H), 3.81 (s, 2H), 3.36 (s, 5H), 2.46-2.37 (m, 1H), 2.23-2.16 (m, 1H), 2.04-1.95 (m, 2H), 1.89-1.78 (m, 2H), 1.69-1.59 (m, 2H), 1.10-1.03 (m, 2H), 0.99-0.93 (m, 2H).

### Example 461: [(2-{6-cyclopropyl-4-[4-fluoro-2-(4-methyl-1,2,4-triazol-3-yl)phenyl]pyridin-2-yl}-7-methyl-1,3-benzoxazol-5-yl)methyl][(2S)-2-methoxypropyl]amine (AHF-1)

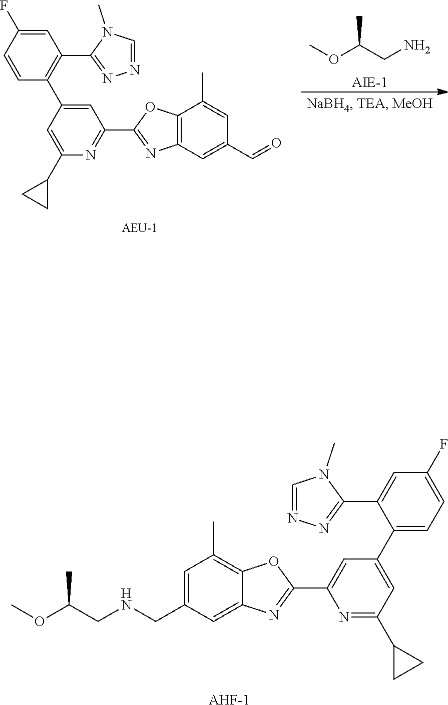

To a stirred solution of 2-{6-cyclopropyl-4-[4-fluoro-2-(4-methyl-1,2,4-triazol-3-yl)phenyl]pyridin-2-yl}-7-methyl-1,3-benzoxazole-5-carbaldehyde (AEU-1) (30 mg, 1 Eq, 66 μmol) and (2S)-2-methoxypropan-1-amine (AIE-1) (12 mg, 2 Eq, 0.13 mmol) in MeOH (5 mL) was added TEA (58 mg, 5 Eq, 0.57 mmol) for 2 h at 60° C. under nitrogen atmosphere. To the above mixture was added NaBH₄ (43 mg, 10 Eq, 1.14 mmol) at 0° C. The resulting mixture was stirred for 2 h at room temperature. The reaction was then quenched by the addition of 2 mL of methyl alcohol at 0° C. The resulting mixture was diluted with water and extracted with dichloromethane (3×15 mL). The combined organic layers were washed with brine (2×15 mL), dried over anhydrous sodium sulfate. After filtration, the filtrate was concentrated under reduced pressure. The crude product was purified by Prep-HPLC with the following conditions: Column: XBridge Prep OBD C18 Column, 30×150 mm, 5 μm; Mobile Phase A: Water (0.1% NH₄HCO₃+0.1% NH₃·H₂O) Mobile Phase B: ACN; Flow rate: 60 mL/min; Gradient: 36 B to 46 B in 8 min; Detector, UV 254/220 nm; RT: 7.18. The product-containing fractions were combined and evaporated partially in vacuo and lyophilized overnight to afford the title compound (AHF-1) (10.7 mg, 20 μmol, 31%, 99.9% Purity) as a white solid. m/z 527.7 (M+H)⁺ (ES+). ¹H NMR (400 MHz, DMSO-d6) δ 8.51 (s, 1H), 7.90-7.83 (m, 1H), 7.74 (d, J=1.5 Hz, 1H), 7.68-7.57 (m, 3H), 7.27 (s, 1H), 7.08 (d, J=1.6 Hz, 1H), 3.91-3.72 (m, 2H), 3.30-3.16 (m, 8H), 2.84-2.73 (m, 1H), 2.54 (s, 3H), 2.22-2.13 (m, 1H), 1.09-0.89 (m, 7H).

### Example 462: [(2-{6-cyclopropyl-4-[4-fluoro-2-(4-methyl-1,2,4-triazol-3-yl)phenyl]pyridin-2-yl}-6-fluoro-7-methyl-1,3-benzoxazol-5-yl)methyl](2-methoxyethyl)amine (AHG-1)

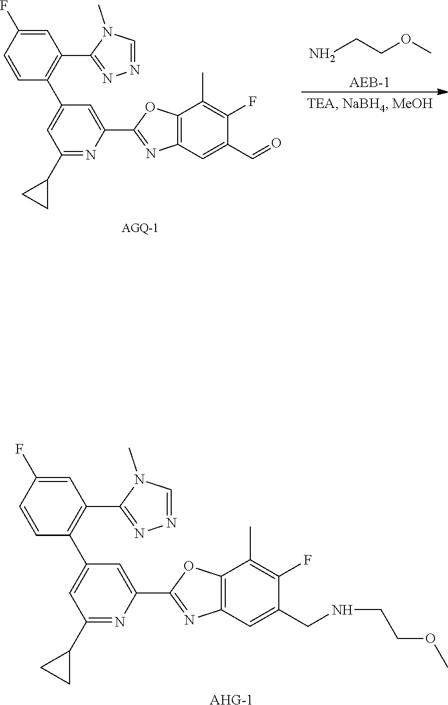

To a stirred solution of 2-{6-cyclopropyl-4-[4-fluoro-2-(4-methyl-1,2,4-triazol-3-yl)phenyl]pyridin-2-yl}-6-fluoro-7-methyl-1,3-benzoxazole-5-carbaldehyde (AGQ-1) (40 mg, 1 Eq, 85 μmol) and 2-methoxyethan-1-amine (AEB-1) (13 mg, 2 Eq, 0.17 mmol) in MeOH (5 mL) was added TEA (43 mg, 5 Eq, 0.43 mmol) for 2 h at 60° C. under nitrogen atmosphere. To the above mixture was added NaBH₄ (32 mg, 10 Eq, 0.85 mmol) at 0° C. The resulting mixture was stirred for 2 h at room temperature. The reaction was then quenched by the addition of 2 mL of methyl alcohol at 0° C. The resulting mixture was diluted with water and extracted with dichloromethane (3×15 mL). The combined organic layers were washed with brine (2×15 mL), dried over anhydrous sodium sulfate. After filtration, the filtrate was concentrated under reduced pressure. The crude product was purified by Prep-HPLC with the following conditions: Column: XBridge Prep OBD C18 Column, 30×150 mm, 5 μm; Mobile Phase A: Water (0.1% NH₄HCO₃+0.1% NH₃·H₂O) Mobile Phase B: ACN; Flow rate: 60 mL/min; Gradient: 36 B to 46 B in 8 min; Detector, UV 254/220 nm; RT: 7.43. The product-containing fractions were combined and evaporated partially in vacuo and lyophilized overnight to afford the title compound (AHG-1) (20 mg, 38 μmol, 44%, 99.6% Purity) as a white solid. m/z 531.5 (M+H)⁺ (ES+). ¹H NMR (400 MHz, Methanol-d4) δ 8.46 (s, 1H), 7.87-7.81 (m, 2H), 7.65 (d, J=6.2 Hz, 1H), 7.58-7.57 (m, 1H), 7.53-7.48 (m, 1H), 7.10 (d, J=1.6 Hz, 1H), 3.96 (d, J=1.4 Hz, 2H), 3.56-3.50 (m, 2H), 3.35 (s, 3H), 3.30 (s, 3H), 2.81 (t, J=5.3 Hz, 2H), 2.54 (d, J=1.7 Hz, 3H), 2.21-2.14 (m, 1H), 1.09-1.01 (m, 4H).

### Example 463: [(2-{6-cyclopropyl-4-[4-fluoro-2-(4-methyl-1,2,4-triazol-3-yl)phenyl]pyridin-2-yl}-6,7-difluoro-1,3-benzoxazol-5-yl)methyl]dimethylamine (AHH-3)

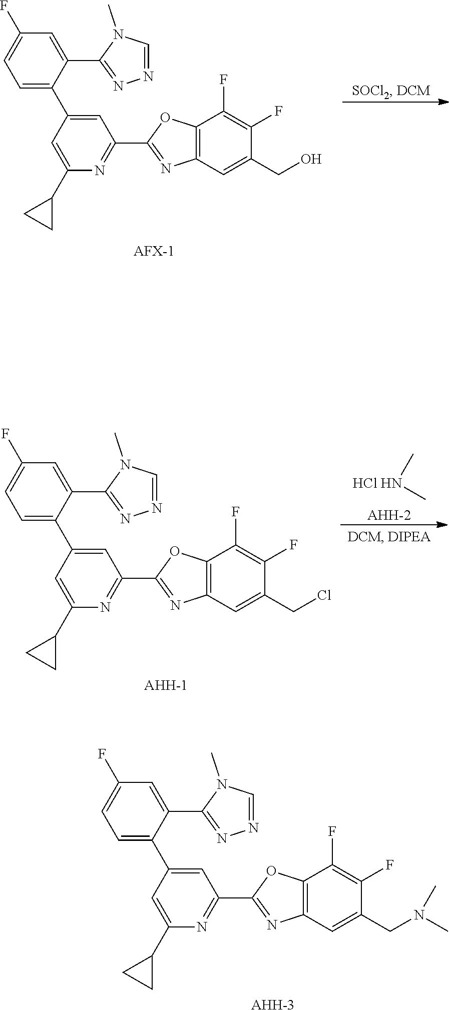

Step 1: 5-(chloromethyl)-2-{6-cyclopropyl-4-[4-fluoro-2-(4-methyl-1,2,4-triazol-3-yl)phenyl]pyridin-2-yl}-6,7-difluoro-1,3-benzoxazole (AHH-1). To a stirred mixture of (2-{6-cyclopropyl-4-[4-fluoro-2-(4-methyl-1,2,4-triazol-3-yl)phenyl]pyridin-2-yl}-6,7-difluoro-1,3-benzoxazol-5-yl)methanol (AFX-1) (40 mg, 1 Eq, 84 μmol) in DCM (5 mg) was added SOCl₂ (0.2 mL) at room temperature. The resulting mixture was stirred for 1 h at room temperature. The reaction was then quenched by the addition of 2 mL of methyl alcohol at 0° C. The resulting mixture was concentrated under reduced pressure. The residue was purified by Prep-TLC with dichloromethane/methyl alcohol (12/1) to afford the sub-title compound (AHH-1) (5.2 mg, 0.01 mmol, 12%, 93% Purity) as a white solid. m/z 496.1/498.1 (M+H)⁺ (ES+).

Step 2: [(2-{6-cyclopropyl-4-[4-fluoro-2-(4-methyl-1,2,4-triazol-3-yl)phenyl]pyridin-2-yl}-6,7-difluoro-1,3-benzoxazol-5-yl)methyl]dimethylamine (AHH-3). To a stirred mixture of the product from step 1 above (AHH-1) (40 mg, 1 Eq, 81 μmol) in DCM (5 mL) was added dimethylamine hydrochloride (AHH-2) (8.6 mg, 1.3 Eq, 0.11 mmol) and DIPEA (6 mg, 0.6 Eq, 49 μmol) at room temperature. The resulting mixture was stirred for 2 h at room temperature. The resulting mixture was concentrated under vacuum. The crude product was purified by Prep-HPLC with the following conditions (Column: XBridge Prep OBD C18 Column, 19*250 mm, 5 μm; Mobile Phase A: Water (0.11% NHLHCO₃), Mobile Phase B: ACN; Flow rate: 25 mL/min; Gradient: 48% B to 70% B in 6 min; Wave Length: 254 nm; RT: 5.08) to afford the title compound (AHH-3) (4.9 mg, 9.7 μmol, 12%, 99.7% Purity) as a white solid. m/z 505.3 (M+H)⁺ (ES+). ¹H NMR (400 MHz, Methanol-d4) δ 8.53-8.45 (m, 1H), 8.00-7.81 (m, 2H), 7.69-7.48 (m, 3H), 7.14 (d, J=7.9 Hz, 1H), 3.73 (d, J=8.2 Hz, 2H), 3.35 (s, 3H), 2.54-2.28 (m, 6H), 2.25-2.10 (m, 1H), 1.09 (d, J=9.4 Hz, 4H).

### Example 464: 1-({[(2-{4-[4-chloro-2-(4-methyl-1,2,4-triazol-3-yl)phenyl]-6-cyclopropylpyridin-2-yl}-7-fluoro-1,3-benzoxazol-5-yl)methyl]amino}methyl)cyclobutan-1-ol (AHI-5)

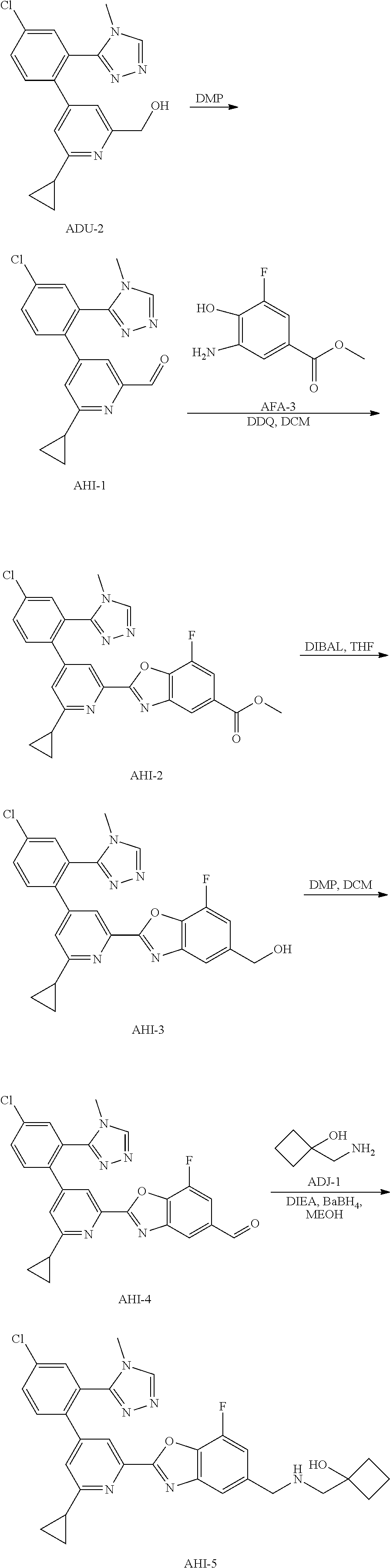

Step 1: 4-[4-chloro-2-(4-methyl-1,2,4-triazol-3-yl)phenyl]-6-cyclopropylpyridine-2-carbaldehyde (AHI-1). To a stirred mixture of {4-[4-chloro-2-(4-methyl-1,2,4-triazol-3-yl)phenyl]-6-cyclopropylpyridin-2-yl}methanol (ADU-2) (400 mg, 1 Eq, 1.17 mmol) in DCM (10 mL) was added DMP (996 mg, 2 Eq, 2.35 mmol) in portions at room temperature under nitrogen atmosphere. The resulting mixture was stirred for 2 h at room temperature under nitrogen atmosphere. The resulting mixture was filtered the filter cake was washed with DCM (3×3 mL). The filtrate was concentrated under reduced pressure. The crude resulting mixture was used in the next step directly without further purification. m/z 339.1/341.1 (M+H)⁺ (ES+).

Step 2: methyl 2-{4-[4-chloro-2-(4-methyl-1,2,4-triazol-3-yl)phenyl]-6-cyclopropylpyridin-2-yl}-7-fluoro-1,3-benzoxazole-5-carboxylate (AHI-2). To a stirred solution of the product from step 1 above (AHI-2) (350 mg, 1 Eq, 1.03 mmol) and methyl 3-amino-5-fluoro-4-hydroxybenzoate (AFA-3) (287 mg, 1.5 Eq, 1.55 mmol) in DCM (15 mL) at room temperature. The resulting mixture was stirred for 1 h at 60° C. under nitrogen atmosphere. The mixture was allowed to cool down to room temperature. To the above mixture was added DDQ (469 mg, 2.07 mmol, 2 Eq) in portions at room temperature. The resulting mixture was stirred for 2 h at room temperature. The resulting mixture was diluted with water and extracted with ethyl acetate (3×50 mL). The combined organic layers were washed with brine (2×50 mL), dried over anhydrous sodium sulfate. After filtration, the filtrate was concentrated under reduced pressure. The crude product was purified by reverse flash column chromatography with the following conditions: Column, C18; mobile phase, Water (0.1% FA) and ACN (35% ACN up to 60% in 20 min); Detector, UV 254/220 nm. The product-containing fractions were combined and concentrated under reduced pressure. This resulted in the sub-title compound (AHI-2) (140 mg, 0.28 mmol, 23%, 92% Purity) as a yellow solid. m/z 504.1/506.1 (M+H)⁺ (ES+).

Step 3: (2-{4-[4-chloro-2-(4-methyl-1,2,4-triazol-3-yl)phenyl]-6-cyclopropylpyridin-2-yl}-7-fluoro-1,3-benzoxazol-5-yl)methanol (AHI-3). To a stirred solution of the product from step 2 above (AHI-2) (300 mg, 1 Eq, 0.60 mmol) in THF (5 mL) was added DIBAl-H (g) in THF (0.43 mL, 1.5 M, 8 Eq, 0.64 mmol) was stirred for 2 hours at 0° C. under nitrogen atmosphere. The reaction was then quenched by the addition of 2 mL of methyl alcohol at 0° C. The resulting mixture was diluted with water and extracted with ethyl acetate (3×20 mL). The combined organic layers were washed with brine (2×20 mL), dried over anhydrous sodium sulfate. After filtration, the filtrate was concentrated under reduced pressure. The residue was purified by Prep-TLC with dichloromethane/methyl alcohol (10/1). This resulted in the sub-title compound (AHI-3) (100 mg, 0.21 mmol, 35%, 90% Purity) as a yellow solid. m/z 476.1/478.1 (M+H)⁺ (ES+).

Step 4: 2-{4-[4-chloro-2-(4-methyl-1,2,4-triazol-3-yl)phenyl]-6-cyclopropylpyridin-2-yl}-7-fluoro-1,3-benzoxazole-5-carbaldehyde (AHI-4). To a stirred solution of the product from step 3 above (AHI-3) (30 mg, 1 Eq, 63 μmol) in DCM (5 mL) was added DMP (29 mg, 1.3 Eq, 69 μmol) at room temperature. The resulting mixture was stirred for 1 h at room temperature. The resulting mixture was filtered the filter cake was washed with ethyl acetate (3×3 mL). The filtrate was concentrated under reduced pressure. This resulted in the sub-title compound (AHI-4) (27 mg, 57 μmol, 77%, 95% Purity) as a yellow oil. m/z 474.1/476.1 (M+H)⁺ (ES+).

Step 5: 1-({[(2-{4-[4-chloro-2-(4-methyl-1,2,4-triazol-3-yl)phenyl]-6-cyclopropylpyridin-2-yl}-7-fluoro-1,3-benzoxazol-5-yl)methyl]amino}methyl)cyclobutan-1-ol (AHI-5). A solution of the product from step 4 above (AHI-4) (26 mg, 1 Eq, 55 μmol) and 1-(aminomethyl)cyclobutan-1-ol (ADJ-1) (8 mg, 1.5 Eq, 83 μmol) in MeOH (3 mL) was added DIPEA (21 mg, 3 Eq, 0.17 mmol) for 2 h at 60° C. under nitrogen atmosphere. The mixture was allowed to cool down to room temperature. To the above mixture was added NaBH₄ (10 mg, 5 Eq, 0.28 mmol) at 0° C. The resulting mixture was stirred for 2 h at room temperature. The reaction was then quenched by the addition of 2 mL of methyl alcohol at 0° C. The resulting mixture was diluted with water and extracted with dichloromethane (3×20 mL). The combined organic layers were washed with brine (2×20 mL), dried over anhydrous sodium sulfate. After filtration, the filtrate was concentrated under reduced pressure. The crude product was purified by Prep-HPLC with the following conditions: Column: XBridge Prep OBD C18 Column, 19*250 mm, 5 μm; Mobile Phase A: Water (0.1% NH₄HCO₃), Mobile Phase B: ACN; Flow rate: 25 mL/min; Gradient: 50% B to 60% B in 6 min; Wave Length: 254 nm. RT: 7.9. The product-containing fractions were combined and evaporated partially in vacuo and lyophilized overnight to afford the title compound (AHI-5) (7.1 mg, 13 μmol, 23%, 98.5% Purity) as a white solid. m/z 559.2/561.2 (M+H)⁺ (ES+). ¹H NMR (400 MHz, Methanol-d4) δ 8.49 (s, 1H), 7.95 (s, 1H), 7.88-7.77 (m, 3H), 7.65 (s, 1H), 7.38 (d, J=11.0 Hz, 1H), 7.14 (s, 1H), 3.98 (s, 2H), 3.33 (s, 3H), 2.73 (s, 2H), 2.20-2.01 (m, 5H), 1.76 (s, 1H), 1.58-1.50 (m, 1H), 1.09 (d, J=6.8 Hz, 4H).

### Example 465: 1-({[(2-{6-cyclopropyl-4-[4-fluoro-2-(4-methyl-1,2,4-triazol-3-yl)phenyl]pyridin-2-yl}-6-fluoro-7-methyl-1,3-benzoxazol-5-yl)methyl]amino}methyl)cyclobutan-1-ol (AHJ-1)

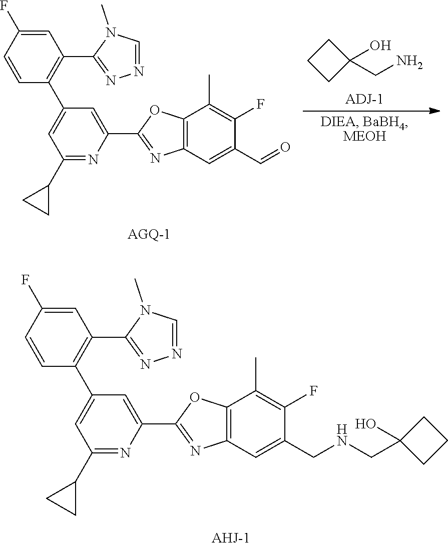

To a stirred solution of 2-{6-cyclopropyl-4-[4-fluoro-2-(4-methyl-1,2,4-triazol-3-yl)phenyl]pyridin-2-yl}-6-fluoro-7-methyl-1,3-benzoxazole-5-carbaldehyde (AGQ-1) (25 mg, 1 Eq, 53 μmol) and 1-(aminomethyl)cyclobutan-1-ol (ADJ-1) (16 mg, 3 Eq, 0.16 mmol) in MeOH (1 mL) was added DIPEA (2 mL) at room temperature. The resulting mixture was stirred for 2 h at 60° C. The mixture was allowed to cool down to room temperature. To the above mixture was added NaBH₄ (20 mg, 10 Eq, 0.53 mmol) at 0° C. The resulting mixture was stirred for additional 30 min at room temperature. The reaction was then quenched by the addition of 2 mL of methyl alcohol at 0° C. The resulting mixture was diluted with water and extracted with ethyl acetate (3×10 mL). The combined organic layers were washed with brine (2×10 mL), dried over anhydrous sodium sulfate. After filtration, the filtrate was concentrated under reduced pressure. The crude product was purified by Prep-HPLC with the following conditions (Column: XBridge BEH C18 OBD Prep Column, 19*250 mm, 5 μm; Mobile Phase A: Water (0.1% NH₄HCO₃), Mobile Phase B: ACN; Flow rate: 25 mL/min; Gradient: 45% B to 55% B in 6 min; Wave Length: 254 nm; RT: 5.3) to afford the title compound (AHJ-1) (3.0 mg, 5.4 μmol, 9.8%, 96.3% Purity) as a white solid. m/z 557.3 (M+H)⁺ (ES+). ¹H NMR (400 MHz, Methanol-d4) δ 8.48 (s, 1H), 7.92-7.80 (m, 2H), 7.70 (d, J=6.3 Hz, 1H), 7.63-7.57 (m, 1H), 7.55-7.50 (m, 1H), 7.13 (d, J=1.6 Hz, 1H), 4.04 (s, 2H), 3.34 (s, 3H), 2.80 (s, 2H), 2.57 (d, J=1.7 Hz, 3H), 2.18-2.08 (m, 3H), 2.09-1.99 (m, 2H), 1.84-1.72 (m, 1H), 1.61-1.50 (m, 1H), 1.19-1.01 (m, 4H).

### Example 466: 1-[({6-fluoro-2-[4′-fluoro-2′-(4-methyl-1,2,4-triazol-3-yl)-[1,1′-biphenyl]-3-yl]-7-methyl-1,3-benzoxazol-5-yl}methyl)amino]-2-methylpropan-2-ol (AHK-4)

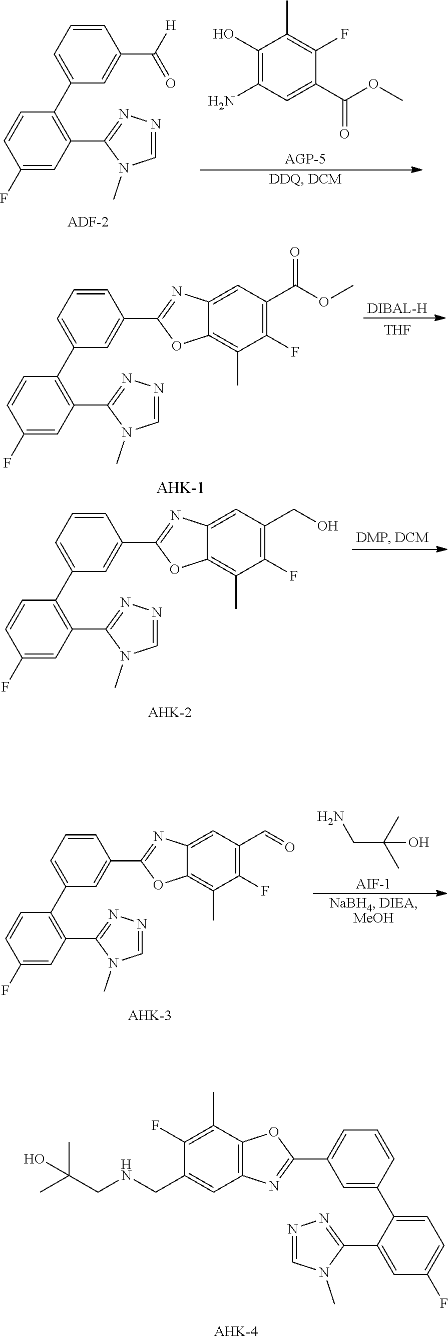

Step 1: methyl 6-fluoro-2-[4′-fluoro-2′-(4-methyl-1,2,4-triazol-3-yl)-[1,1′-biphenyl]-3-yl]-7-methyl-1,3-benzoxazole-5-carboxylate (AHK-1). To a stirred solution of 4′-fluoro-2′-(4-methyl-1,2,4-triazol-3-yl)-[1,1′-biphenyl]-3-carbaldehyde (ADF-2) (120 mg, 1 Eq, 0.43 mmol) and methyl 5-amino-2-fluoro-4-hydroxy-3-methylbenzoate (AGP-5) (102 mg, 1.2 Eq, 0.51 mmol) in DCM (15 mL) at room temperature. The resulting mixture was stirred for 1 h at 60° C. under nitrogen atmosphere. The mixture was allowed to cool down to room temperature. To the above mixture was added DDQ (194 mg, 2 Eq, 0.85 mmol) in portions at room temperature. The resulting mixture was stirred for 2 h at room temperature. The resulting mixture was diluted with water and extracted with ethyl acetate (3×30 mL). The combined organic layers were washed with brine (2×30 mL), dried over anhydrous sodium sulfate. After filtration, the filtrate was concentrated under reduced pressure. The crude product was purified by reverse flash column chromatography with the following conditions: Column, C18; mobile phase, Water (0.1% FA) and ACN (35% ACN up to 60% in 20 min); Detector, UV 254/220 nm. The product-containing fractions were combined and concentrated under reduced pressure. This resulted in the sub-title compound (AHK-1) (70 mg, 0.15 mmol, 33%, 92% Purity) as a yellow solid. m/z 461.1 (M+H)⁺ (ES+)

Step 2: {6-fluoro-2-[4′-fluoro-2′-(4-methyl-1,2,4-triazol-3-yl)-[1,1′-biphenyl]-3-yl]-7-methyl-1,3-benzoxazol-5-yl}methanol (AHK-2). To a stirred solution of the product from step 1 above (AHK-1) (50 mg, 1 Eq, 0.11 mmol) in THF (5 mL) was added DIBAl-H (g) in THF (0.58 mL, 1.5 M, 8 Eq, 0.87 mmol) was stirred for 2 h at 0° C. under nitrogen atmosphere. The reaction was then quenched by the addition of 2 mL of ice water at 0° C. The resulting mixture was diluted with water and extracted with ethyl acetate (3×10 mL). The combined organic layers were washed with brine (2×10 mL), dried over anhydrous sodium sulfate. After filtration, the filtrate was concentrated under reduced pressure. The residue was purified by Prep-TLC with dichloromethane/methyl alcohol (10/1). This resulted in the sub-title compound (AHK-2) (30 mg, 69 μmol, 60%, 90% Purity) as a yellow solid. m/z 433.1 (M+H)⁺ (ES+)

Step 3: 6-fluoro-2-[4′-fluoro-2′-(4-methyl-1,2,4-triazol-3-yl)-[1,1′-biphenyl]-3-yl]-7-methyl-1,3-benzoxazole-5-carbaldehyde (AHK-3). To a stirred solution of the product from step 2 above (AHK-2) (40 mg, 1 Eq, 93 μmol) in DCM (5 mL) was added DMP (78 mg, 2 Eq, 0.19 mmol) at room temperature. The resulting mixture was stirred for 1 h at room temperature. The resulting mixture was filtered the filter cake was washed with ethyl acetate (3×3 mL). The filtrate was concentrated under reduced pressure. This resulted in the sub-title compound (AHK-3) (40 mg, 93 μmol, 94%, 92% Purity) as a yellow oil. m/z 431.1 (M+H)⁺ (ES+)

Step 4: 1-[({6-fluoro-2-[4′-fluoro-2′-(4-methyl-1,2,4-triazol-3-yl)-[1,1′-biphenyl]-3-yl]-7-methyl-1,3-benzoxazol-5-yl}methyl)amino]-2-methylpropan-2-ol (AHK-4). A solution of the product from step 3 above (AHK-3) (30 mg, 1 Eq, 70 μmol) and 1-amino-2-methylpropan-2-ol (AIF-1) (7.5 mg, 1.2 Eq, 84 μmol) in MeOH (5 mL) was added DIPEA (27 mg, 3 Eq, 0.21 mmol) for 2 h at 60° C. under nitrogen atmosphere. The mixture was allowed to cool down to room temperature. To the above mixture was added NaBH₄ (13 mg, 5 Eq, 0.35 mmol) at 0° C. The resulting mixture was stirred for 2 h at room temperature. The reaction was then quenched by the addition of 2 mL of methyl alcohol at 0° C. The resulting mixture was diluted with water and extracted with dichloromethane (3×10 mL). The combined organic layers were washed with brine (2×10 mL), dried over anhydrous sodium sulfate. After filtration, the filtrate was concentrated under reduced pressure. The crude product was purified by Prep-HPLC with the following conditions: Column: XBridge BEH C18 OBD Prep Column, 19*250 mm, 5 μm; Mobile Phase A: Water (0.1% NH₄HCO₃), Mobile Phase B: ACN; Flow rate: 25 mL/min; Gradient: 42% B to 47% B in 6 min; Wave Length: 254 nm; RT: 7.9. The product-containing fractions were combined and evaporated partially in vacuo and lyophilized overnight to afford the title compound (AHK-4) (10.0 mg, 20.0 μmol, 28%, 97.1% Purity) as a white solid. m/z 504.2 (M+H)⁺ (ES+). ¹H NMR (400 MHz, Methanol-d4) δ 8.41 (d, J=2.8 Hz, 1H), 8.20 (d, J=7.6 Hz, 1H), 8.05 (s, 1H), 7.85-7.78 (m, 1H), 7.63 (d, J=6.2 Hz, 1H), 7.61-7.54 (m, 2H), 7.52-7.42 (m, 2H), 3.97 (s, 2H), 3.20 (d, J=2.8 Hz, 3H), 2.60 (d, J=2.7 Hz, 2H), 2.55 (s, 3H), 1.24 (d, J=2.7 Hz, 6H).

### Example 467: 2-(2-{6-cyclopropyl-4-[4-fluoro-2-(4-methyl-1,2,4-triazol-3-yl)phenyl]pyridin-2-yl}-6,7-difluoro-1,3-benzoxazol-5-yl)propan-2-ol (AHL-7)

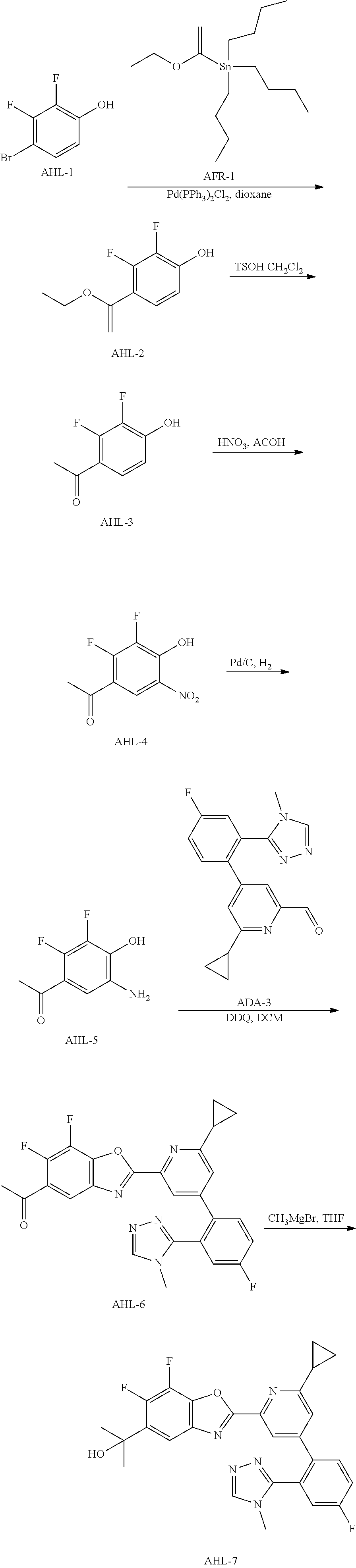

Step 1: 4-(1-ethoxyethenyl)-2,3-difluorophenol (AHL-2). To a stirred solution of 4-bromo-2,3-difluorophenol (AHL-1) (5.00 g, 1 Eq, 23.9 mmol) and tributyl[(prop-1-en-2-yloxy)methyl]stannane (AFR-1) (10.4 g, 1.2 Eq, 28.7 mmol) in dioxane (80 mL) were added Pd(PPh₃)₂Cl₂ (1.68 g, 0.1 Eq, 2.39 mmol) at room temperature under nitrogen atmosphere. The resulting mixture was stirred for overnight at 100° C. under nitrogen atmosphere. The mixture was allowed to cool down to room temperature. The resulting mixture was concentrated under reduced pressure. The residue was applied on a silica gel column chromatography with petroleum ether/ethyl acetate (3/1) to afford the sub-title compound (AHL-2) (3.34 g, 16.7 mmol, 70%, 82% Purity) as a yellow solid. m/z 201.1 (M+H)⁺ (ES+)

Step 2: 1-(2,3-difluoro-4-hydroxyphenyl)ethanone (AHL-3). To a stirred solution of the product from step 1 above (AHL-2) (3.34 g, 1 Eq, 16.7 mmol) and TSOH (1.74 g, 2 Eq, 33.4 mmol) in DCM (50 mL) at room temperature. The resulting mixture was stirred for overnight at room temperature. The resulting mixture was concentrated under reduced pressure. The residue was applied on a silica gel column chromatography with petroleum ether/ethyl acetate (3/1) to afford the sub-title compound (AHL-3) (2.69 g, 15.6 mmol, 94%, 90% Purity) as a pink solid. m/z 173.0 (M+H)⁺ (ES+).

Step 3: 1-(2,3-difluoro-4-hydroxy-5-nitrophenyl)ethanone (AHL-4). Into a 250 mL vial were added the product from step 2 above (AHL-3) (2.60 g, 11 Eq, 5.1 mmol), HNO₃ (27 mL), fuming HNO₃ (13.5 mL) and AcOH (80 mL) at 0° C. The resulting mixture was stirred for additional 2 h at room temperature. The resulting mixture was diluted with water and extracted with ethyl acetate (3×200 mL). The combined organic layers were washed with brine (2×200 mL), dried over anhydrous sodium sulfate. After filtration, the filtrate was concentrated under reduced pressure. The residue was applied on a silica gel column chromatography with dichloromethane/methyl (10/1) to afford the sub-title compound (AHL-4) (730 mg, 3.36 mmol, 22%, 90% Purity) as a yellow solid. m/z 218.0 (M+H)⁺ (ES+).

Step 4: 1-(5-amino-2,3-difluoro-4-hydroxyphenyl)ethanone (AHL-5). To a stirred solution of the product from step 3 above (AHL-4) (1.50 g, 1 Eq, 6.91 mmol) in MeOH (30 mL) was added Pd/C (147 mg, 10% Wt, 0.2 Eq, 1.38 mmol) at room temperature under nitrogen atmosphere. The resulting mixture was stirred for 1 h at room temperature under hydrogen atmosphere. The resulting mixture was filtered the filter cake was washed with MeOH (3×10 mL). The filtrate was concentrated under reduced pressure. This resulted in the sub-title compound (AHL-5) (1.3 g, 6.95 mmol, 93%, 90% Purity) as a brown solid. m/z 188.0 (M+H)⁺ (ES+).

Step 5: 1-(2-{6-cyclopropyl-4-[4-fluoro-2-(4-methyl-1,2,4-triazol-3-yl)phenyl]pyridin-2-yl}-6,7-difluoro-1,3-benzoxazol-5-yl)ethanone (AHL-6). To a stirred mixture of 6-cyclopropyl-4-[4-fluoro-2-(4-methyl-1,2,4-triazol-3-yl)phenyl]pyridine-2-carbaldehyde (ADA-3) (300 mg, 1 Eq, 0.93 mmol) and the product from step 4 above (AHL-5) (209 mg, 1.2 Eq, 1.12 mmol) in DCM (10 mL) at room temperature. The resulting mixture was stirred for 1 h at 60° C. under nitrogen atmosphere. The mixture was allowed to cool down to room temperature. To the above mixture was added DDQ (423 mg, 2 Eq, 1.86 mmol) in portions at room temperature. The resulting mixture was stirred for 2 h at room temperature. The resulting mixture was concentrated under reduced pressure. The crude product was purified by reverse flash column chromatography with the following conditions: Column, C18; mobile phase, Water (0.1% FA) and ACN (0% ACN up to 50% in 20 min); Detector, UV 254/220 nm. This resulted in the sub-title compound (AHL-6) (260 mg, 0.53 mmol, 57%, 90% Purity) as a white solid. m/z 490.1 (M+H)⁺ (ES+).

Step 6: 2-(2-{6-cyclopropyl-4-[4-fluoro-2-(4-methyl-1,2,4-triazol-3-yl)phenyl]pyridin-2-yl}-6,7-difluoro-1,3-benzoxazol-5-yl)propan-2-ol (AHL-7). To a stirred mixture of the product from step 5 above (AHL-6) (90 mg, 1 Eq, 0.18 mmol) in THF (4 mL) was added CH₃MgBr (110 mg, 5 Eq, 0.92 mmol) in portions at 0° C. under nitrogen atmosphere. The resulting mixture was stirred for 1 h at 0° C. under nitrogen atmosphere. The reaction was then quenched by the addition of 2 mL of ice water at 0° C. The resulting mixture was concentrated under reduced pressure. The residue was purified by Prep-TLC with dichloromethane/methyl alcohol (10/1). The crude product was purified by Prep-HPLC with the following conditions (Column: XBridge Prep OBD C18 Column, 30*150 mm, 5 μm; Mobile Phase A: Water (0.1% NH₄HCO₃+0.1% NH₃·H₂O), Mobile Phase B: ACN; Flow rate: 60 mL/min; Gradient: 10% B to 80% B in 8 min; Wave Length: 254/220 nm; RT: 6.8) to afford the title compound (AHL-7) (3.8 mg, 7.5 μmol, 4.1%, 99.7% Purity) as a white solid. m/z 506.1 (M+H)⁺ (ES+). ¹H NMR (400 MHz, Methanol-d4) δ 8.49 (s, 1H), 7.92 (d, J=1.5 Hz, 1H), 7.90-7.81 (m, 2H), 7.63-7.48 (m, 2H), 7.13 (d, J=1.6 Hz, 1H), 3.33 (s, 3H), 2.22-2.13 (m, 1H), 1.67 (d, J=1.2 Hz, 6H), 1.12-1.05 (m, 4H).

### Example 468: 1-(2-{6-cyclopropyl-4-[4-fluoro-2-(4-methyl-1,2,4-triazol-3-yl)phenyl]pyridin-2-yl}-7-fluoro-1,3-benzoxazol-5-yl)methanamine (AHM-3)

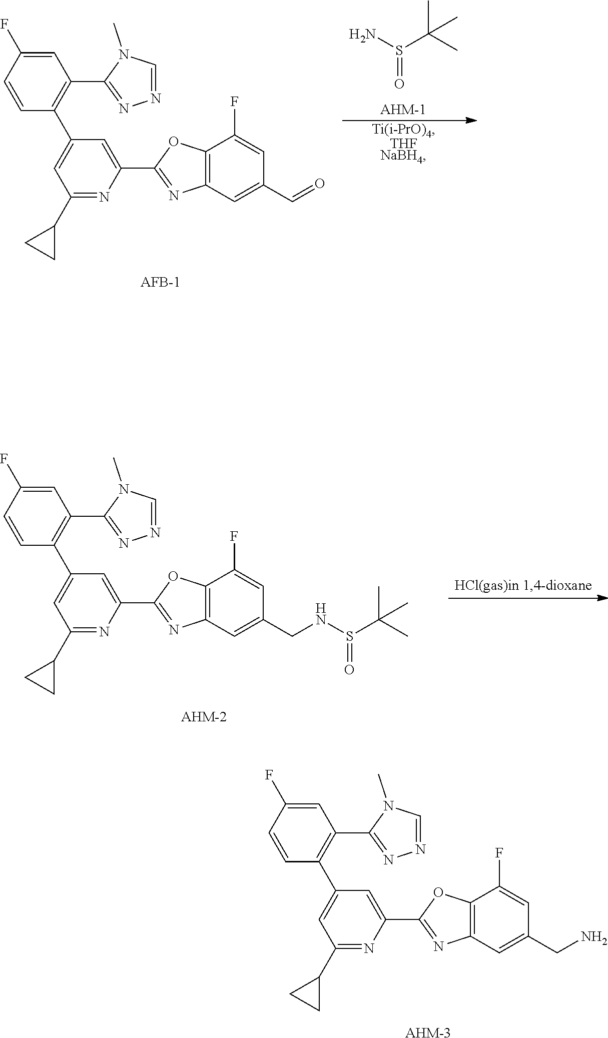

Step 1: N-((2-(6-cyclopropyl-4-(4-fluoro-2-(4-methyl-4H-1,2,4-triazol-3-yl)phenyl)pyridin-2-yl)-7-fluorobenzo[d]oxazol-5-yl)methyl)-2-methylpropane-2-sulfinamide (AHM-2). To a stirred mixture of 2-{6-cyclopropyl-4-[4-fluoro-2-(4-methyl-1,2,4-triazol-3-yl)phenyl]pyridin-2-yl}-7-fluoro-1,3-benzoxazole-5-carbaldehyde (AFB-1) (100 mg, 1 Eq, 0.22 mmol) and tert-butanesulfinamide (AHM-1) (132 mg, 5 Eq, 1.10 mmol) in THF (5 mL) were added Ti(Oi-Pr)₄ (186 mg, 3 Eq, 0.66 mmol) at 0° C. The resulting mixture was stirred for overnight at 60° C. The mixture was allowed to cool down to room temperature. To the above mixture was added NaBH₄ (25 mg, 3 Eq, 0.66 mmol) at 0° C. The resulting mixture was stirred for additional 1 h at room temperature. The reaction was then quenched by the addition of 2 mL of methyl alcohol at 0° C. The resulting mixture was concentrated under reduced pressure. The residue was purified by Prep-TLC with dichloromethane/methyl alcohol (10/1). This resulted in the sub-title compound (AHM-2) (20 mg, 36 μmol, 16%, 95% Purity) as an off-white solid. m/z 563.2 (M+H)+(ES+).

Step 2: 1-(2-{6-cyclopropyl-4-[4-fluoro-2-(4-methyl-1,2,4-triazol-3-yl)phenyl]pyridin-2-yl}-7-fluoro-1,3-benzoxazol-5-yl)methanamine (AHM-3). To a stirred mixture of the product from step 1 above (AHM-2) (20 mg, 1 Eq, 36 μmol) in THF (4 mL) was added HCl (g) in 1,4-dioxane (90 μL, 4 M, 10 Eq, 0.36 mmol) in portions at room temperature. The resulting mixture was stirred for additional 1 h at room temperature. The resulting mixture was concentrated under vacuum. The residue was purified by Prep-TLC with dichloromethane/methyl alcohol (10/1). The crude product was purified by Prep-HPLC with the following conditions (Column: XBridge Prep OBD C18 Column, 30*150 mm, 5 μm; Mobile Phase A: Water (0.1% NH₄HCO₃+0.1% NH₃·H₂O), Mobile Phase B: ACN; Flow rate: 60 mL/min; Gradient: 25% B to 45% B in 9 min; Wave Length: 254/220 nm; RT: 8.83) to afford the title compound (AHM-3) (2.7 mg, 5.9 μmol, 17%, 97.9% Purity) as a white solid. m/z 459.1 (M+H)⁺ (ES+). ¹H NMR (400 MHz, Methanol-d4) δ 8.48 (s, 1H), 7.94 (d, J=1.6 Hz, 1H), 7.85 (m, 1H), 7.63-7.59 (m, 2H), 7.53 (m, 1H), 7.34 (d, J=11.0 Hz, 1H), 7.13 (d, J=1.6 Hz, 1H), 3.97 (s, 2H), 3.33 (s, 3H), 2.23-2.15 (m, 1H), 1.13-1.05 (m, 4H).

### Example 469: 1-({[(2-{6-cyclopropyl-4-[4-fluoro-2-(4-methyl-1,2,4-triazol-3-yl)phenyl]pyridin-2-yl}-7-(trifluoromethyl)-1,3-benzoxazol-5-yl)methyl]amino}methyl)cyclopentane-1-carbonitrile (AHN-2)

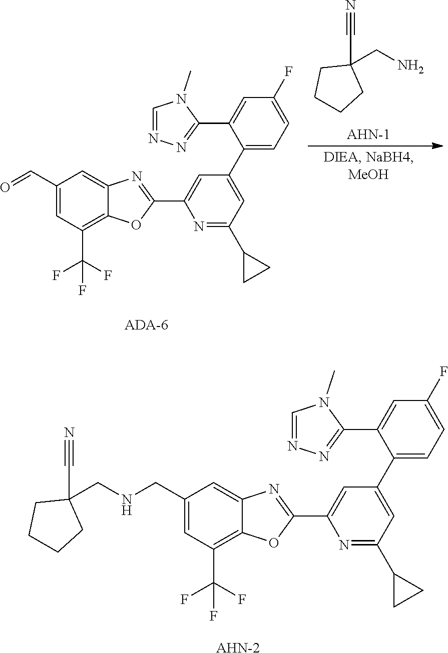

A solution of 2-{6-cyclopropyl-4-[4-fluoro-2-(4-methyl-1,2,4-triazol-3-yl)phenyl]pyridin-2-yl}-7-(trifluoromethyl)-1,3-benzoxazole-5-carbaldehyde (ADA-6) (20 mg, 1 Eq, 39 μmol), 1-(aminomethyl)cyclopentane-1-carbonitrile (AHN-1) (7.3 mg, 1.5 Eq, 58 μmol) and DIPEA (10.2 mg, 2 Eq, 78 μmol) in MeOH (8 mL) was stirred for overnight at 60° C. The mixture was allowed to cool down to room temperature. To the above mixture was added NaBH₄ (3.0 mg, 2 Eq, 78 μmol) in portions at 0° C. The resulting mixture was stirred for additional 1 h at room temperature. The reaction was then quenched by the addition of 2 mL of methyl alcohol at 0° C. The resulting mixture was concentrated under reduced pressure. The residue was purified by Prep-TLC with dichloromethane/methyl alcohol (10/1). The crude product was purified by Prep-HPLC with the following conditions (Column: YMC-Actus Triart C18, 30*150 mm, 5 μm; Mobile Phase A: Water (0.1% NH₄HCO₃), Mobile Phase B: ACN; Flow rate: 60 mL/min; Gradient: 50% B to 70% B in 8 min; Wave Length: 254/210 nm; RT: 7.35) to afford the title compound (AHN-2) (4.2 mg, 6.8 μmol, 17%, 99.5% Purity) as a white solid. m/z 616.3 (M+H)⁺ (ES+). ¹H NMR (400 MHz, DMSO-d6) δ 8.50 (s, 1H), 8.15 (s, 1H), 7.91-7.83 (m, 2H), 7.74 (d, J=1.6 Hz, 1H), 7.69-7.59 (m, 2H), 7.16 (d, J=1.5 Hz, 1H), 3.98 (d, J=5.2 Hz, 2H), 3.33 (s, 3H), 2.90-2.80 (m, 1H), 2.64 (d, J=7.0 Hz, 2H), 2.23-2.15 (m, 1H), 2.00-1.92 (m, 2H), 1.77-1.58 (m, 6H), 1.08-1.02 (m, 2H), 0.96-0.90 (m, 2H).

### Example 470: 1-((((2-(6-cyclopropyl-4-(4-fluoro-2-(4-methyl-4H-1,2,4-triazol-3-yl)phenyl)pyridin-2-yl)-7-(trifluoromethyl)benzo[d]oxazol-5-yl)methyl)(ethyl)amino)methyl)cyclopentane-1-carbonitrile (AHN-3)

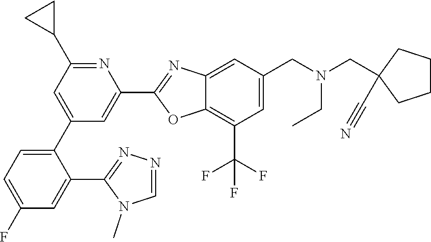 [SMILES: CCN(Cc1cc(C(F)(F)F)c2oc(-c3cc(-c4ccc(F)cc4-c4nncn4C)cc(C4CC4)n3)nc2c1)CC1(C#N)CCCC1]

The titled compound (AHN-3) was prepared according to the methods described herein. m/z 616.3 (M+H)+(ES+).

### Example 471: Cbl-b Biochemical Assay (TR-FRET)

Recombinant human Cbl-b (aa 36-427) was expressed in E coli, purified and biotinylated in vitro. The protein was diluted to 12 nM in freshly prepared assay buffer consisting of 50 mM HEPES, pH 7.0, 100 mM NaCl, 5 mM MgCl2, 0.01% Triton-X 100, 0.01% BSA and 1 mM DTT.

Recombinant human Src (aa 254-536)-GSSGSS-Zap-70 (aa 281-297) fusion protein was expressed in E coli and purified. Protein was diluted in assay buffer to 2-5 nM and supplemented with ATP to 1 mM.

Fluorescein-BODIPY labeled UBED2D(C85K)-Ub was prepared by conjugating ubiquitin (Ub) labeled at its N-terminus with Fluorescein-BODIPY maleimide (ThermoFisher Catalog no B10250) to E. coli expressed and purified UBED2D(C85K) [see Dou et al Nature Structural and Molecular Biology 8: 982-987, 2013]. Recombinant human UBE2D2(C85K) was expressed in E coli, purified and ubiquitinated and Bodipy labelled in vitro. Protein was diluted to 200 nM in assay buffer without MgCl₂. (or Cisbio PPI buffer) Streptavidin-Terbium was added to 2 nM and EDTA to 10 mM, to provide a binding assay mix.

Compounds were dissolved in DMSO and diluted to prepare a ten-point dilution series. 100 nl of each compound concentration was dispensed in duplicate in a 384 well black assay plate using acoustic dispensing. Wells for maximum signal controls received 100 nl of DMSO only and wells for minimum signal controls received 100 nl of a reference inhibitor compound at a final assay concentration of 100 mM to produce 100% inhibition.

5 μl of diluted Cbl-b enzyme was added to all wells of the assay plate and incubated at RT for 30-60 min. The enzyme assay was initiated by addition of 5 μl of Src-Zap/ATP mix to all wells, and the plate incubated at RT for 60 min. The enzyme reaction was terminated and the binding reaction was initiated by adding 10 μl of binding assay mix to all wells and incubating the plate at RT for 60 min prior to assay read.

Final assay conditions consisted of 6 nM Cbl-b, 1-2.5 nM Src-Zap70, 0.5 mM ATP, 1% (v/v) DMSO (enzyme reaction) and 100 nM UBE2D2(C85K)-Ub-FL-BODIPY, 5 mM EDTA, 1 nM Streptavidin-Tb (binding reaction).

The HTRF assay signal was measured at 520 nm on an Envision plate reader, with reference signal at 485 or 620 nm. Data was normalized using maximum and minimum assay controls: % Inhibition=100−(100*((maximum control)−unknown)/(maximum control−minimum control)). A 4-parameter dose-response equation was used to fit the normalized dose-response data and derive an IC50 for test compounds.

Table 2 shows the activity of selected compounds of this invention in the TR-FRET biochemical assay. Compounds having an activity designated as “A” provided an IC₅₀≤0.2 μM; compounds having an activity designated as “B” provided an IC₅₀>0.20 μM. but≤1 μM; compounds designated as “C” provided an IC₅₀>1 μM but≤10 μM; compounds designated as “D” provided an IC₅₀>10 μM but<50 μM.

TABLE 2
Cbl-b Biochemical Assay (TR-FRET) Results

| Compound | TR-FRET hCbl-b | Compound | TR-FRET hCbl-b |
|---|---|---|---|
| ID | IC₅₀ (μM) | ID | IC₅₀ (μM) |
| AA-2 | A | AAM-2 | C |
| AAB-9 | A | AAN-3 | D |
| AAC-5 | C | AAO-4 | D |
| AAD-1 | A | AAP-7 | A |
| AAE-1 | A | AAQ-2 | B |
| AAG-4 | B | AAR-2 | A |
| AAI-5 | B | AAS-2 | A |
| AAJ-1 | C | AAT-14 | C |
| AAK-1 | B | AAU-6 | B |
| AAL-5 | A | AAV-2 | C |
| AAW-5 | A | ACA-2 | D |
| AAX-12 | A | ACB-1 | B |
| AAY-2 | A | ACC-1 | B |
| AAZ-10 | A | ACE-1 | D |
| AB-2 | A | ACF-1 | B |
| ABA-1 | A | ACG-3 | B |
| ABB-2 | D | ACH-5 | D |
| ABC-4 | D | ACJ-5 | D |
| ABD-9 | D | ACK-3 | B |
| ABE-2 | D | ACL-1 | B |
| ABF-4 | D | ACM-1 | B |
| ABG-3 | D | ACN-1 | B |
| ABH-4 | D | ACO-1 | B |
| ABI-7 | D | ACP-4 | D |
| ABJ-2 | D | ACQ-3 | B |
| ABK-1 | B | ACR-3 | D |
| ABL-1 | B | ACT-3 | D |
| ABM-2 | D | ACU-2 | D |
| ABN-2 | D | ACV-5 | D |
| ABO-1 | B | ACW-2 | D |
| ABP-2 | D | ACZ-8 | A |
| ABQ-2 | D | ADA-6 | A |
| ABR-2 | A | ADB-9 | A |
| ABS-2 | D | ADC-3 | C |
| ABT-2 | C | ADD-1 | B |
| ABU-2 | D | ADE-3 | A |
| ABV-1 | D | ADF-2 | A |
| ABW-1 | D | ADG-1 | A |
| ABX-3 | D | ADH-1 | A |
| ABZ-2 | D | ADI-3 | A |
| AC-2 | A | ADJ-2 | C |
| ADK-1 | C | AG-2 | A |
| ADL-2 | A | AH-2 | A |
| ADM-14 | A | AI-2 | A |
| ADN-1 | A | AJ-3 | D |
| ADO-1 | A | AK-2 | A |
| ADP-1 | D | AL-2 | A |
| ADQ-1 | C | AM-2 | A |
| ADR-1 | A | AN-2 | A |
| ADS-1 | A | AO-2 | A |
| ADT-12 | A | AP-2 | A |
| ADU-2 | A | AQ-6 | A |
| ADV-2 | A | AR-2 | D |
| ADW-1 | A | AS-12 | D |
| ADX-3 | D | AT-1 | D |
| ADY-2 | A | AU-1 | B |
| ADZ-1 | A | AV-9 | B |
| AEA-1 | D | AW-4 | D |
| AEB-1 | C | AX-3 | D |
| AEC-2 | A | AY-5 | D |
| AED-3 | D | AZ-1 | D |
| AEE-1 | C | BA-1 | C |
| AEF-2 | A | BB-1 | D |
| AEG-8 | A | BC-1 | D |
| AEH-2 | A | BD-1 | D |
| AEI-1 | A | BE-2 | D |
| AEJ-4 | B | BF-3 | D |
| AEK-2 | C | BG-1 | D |
| AEL-1 | B | BH-3 | D |
| AD-2 | A | BI-1 | B |
| AE-2 | A | BJ-1 | A |
| AF-2 | A | BK-2 | A |
| BL-2 | A | CR-2 | A |
| BM-2 | A | CR-3 | A |
| BN-2 | A | CS-1 | A |
| BO-2 | A | CS-4 | D |
| BP-1 | A | CT-2 | A |
| BQ-1 | A | CU-2 | A |
| BR-1 | A | CV-2 | A |
| BS-1 | A | CW-5 | A |
| BT-1 | A | CX-1 | A |
| BU-1 | A | CY-1 | A |
| BV-1 | A | CZ-2 | A |
| BW-3 | D | DA-1 | A |
| BX-2 | B | DB-2 | B |
| BY-1 | A | DC-3 | C |
| BZ-1 | C | DD-2 | A |
| CA-2 | D | DE-2 | A |
| CB-1 | D | DF-2 | A |
| CC-2 | A | DG-2 | A |
| CD-2 | A | DH-2 | A |
| CF-2 | D | DI-1 | A |
| CG-4 | D | DJ-2 | A |
| CH-2 | D | DK-1 | A |
| CH-3 | D | DL-1 | B |
| CJ-1 | A | DM-2 | A |
| CK-2 | A | DN-2 | A |
| CL-2 | A | DO-6 | A |
| CM-1 | A | DO-7 | A |
| CN-5 | A | DP-5 | A |
| CO-2 | A | DP-6 | A |
| CP-4 | A | DQ-1 | A |
| CQ-1 | A | DR-2 | A |
| DS-2 | A | ET-8 | A |
| DT-2 | A | EU-2 | A |
| DT-3 | A | EV-9 | A |
| DU-2 | A | EW-2 | A |
| DV-2 | A | EX-6 | B |
| DW-3 | C | EY-2 | A |
| DX-2 | A | EZ-3 | A |
| DY-1 | A | FA-1 | A |
| DZ-1 | B | FB-2 | A |
| EA-1 | A | FC-2 | A |
| EA-2 | A | FD-2 | A |
| EB-1 | A | FE-3 | C |
| EC-1 | A | FF-6 | A |
| ED-7 | A | FG-2 | A |
| EE-2 | A | FH-2 | C |
| EF-1 | A | FI-2 | A |
| EG-2 | A | FJ-2 | A |
| EG-3 | A | FK-2 | A |
| EH-1 | A | FL-2 | A |
| EI-4 | A | FM-2 | A |
| EJ-2 | A | FN-1 | A |
| EK-5 | C | FO-2 | A |
| EL-1 | A | FP-1 | A |
| EM-2 | A | FQ-1 | B |
| EN-2 | A | FR-1 | A |
| EO-2 | A | FS-2 | A |
| EP-2 | A | FT-1 | A |
| EQ-4 | B | FU-1 | A |
| ER-2 | A | FV-2 | A |
| ES-4 | A | FV-3 | A |
| ES-6 | A | FW-3 | A |
| FX-2 | A | F-5 | C |
| FY-1 | A | G-1 | D |
| FZ-5 | C | H-1 | D |
| GA-1 | A | I-2 | D |
| GB-2 | A | J-1 | D |
| GC-2 | A | K-2 | D |
| GD-1 | A | L-4 | C |
| GE-2 | A | M-5 | B |
| GF-2 | A | N-1 | A |
| GG-2 | A | 0-4 | B |
| GH-1 | A | P-6 | A |
| GI-2 | B | Q-3 | B |
| GJ-2 | B | R-3 | A |
| GK-5 | A | S-1 | B |
| GL-2 | C | T-5 | A |
| GM-2 | B | U-1 | A |
| GN-5 | D | V-1 | A |
| GO-2 | A | W-2 | A |
| GP-2 | A | X-1 | B |
| GU-1 | A | X-2 | A |
| GV-1 | A | Y-3 | A |
| GW-2 | A | Z-2 | A |
| GX-1 | A | AFY-2 | A |
| GY-1 | B | AGB-1 | A |
| GZ-1 | A | AHL-7 | B |
| HA-3 | A | AHM-3 | A |
| HB-1 | A | AHH-3 | A |
| HC-2 | A | AHI-5 | A |
| HD-2 | A | AHJ-1 | A |
| HE-4 | A | AHK-4 | A |
| HF-1 | A | JO-1 | A |
| AHF-1 | A | JI-3 | A |
| AHG-1 | A | JJ-3 | A |
| AGF-1 | A | JK-7 | A |
| AHC-1 | B | AEZ-1 | A |
| AHD-1 | B | AGO-2 | A |
| AHA-2 | A | JG-1 | A |
| AGZ-2 | A | JH-1 | A |
| AHB-1 | A | AGM-8 | B |
| AHE-2 | A | AGN-3 | A |
| AGX-1 | A | JB-2 | A |
| AGY-1 | A | JC-1 | B |
| IT-1 | A | JD-1 | A |
| JR-2 | A | JE-1 | A |
| JS-1 | A | JF-1 | A |
| JT-1 | A | AGG-2 | A |
| JU-1 | A | AGI-1 | A |
| AGU-1 | A | AGH-4 | B |
| JL-1 | A | AGJ-1 | A |
| JM-4 | A | AGK-1 | A |
| JN-1 | A | AGL-1 | A |
| JP-1 | A | IZ-1 | A |
| JQ-1 | A | IY-1 | A |
| AGS-4 | A | IU-2 | A |
| AGT-1 | A | IV-2 | A |
| AFI-1 | A | IW-1 | A |
| AFQ-6 | A | IX-1 | A |
| AFR-1 | A | AGD-1 | A |
| AGP-7 | B | AGE-1 | A |
| AGR-9 | B | IS-1 | A |
| AEO-1 | A | AGA-1 | A |
| AGQ-2 | A | AGC-2 | A |
| IR-1 | A | IE-1 | A |
| AFX-1 | B | IF-2 | A |
| AFZ-2 | B | IG-6 | A |
| IP-7 | A | AET-1 | B |
| IQ-8 | A | AES-3 | C |
| AEU-2 | A | IA-1 | A |
| AFF-1 | A | IB-7 | A |
| AFU-1 | A | IC-1 | A |
| AFV-1 | A | AFG-1 | A |
| AFW-1 | C | AFH-2 | A |
| DB-1 | D | HV-3 | B |
| IN-2 | C | HW-2 | A |
| IO-4 | C | HX-8 | A |
| AFS-2 | A | HY-3 | A |
| AFT-1 | A | HZ-2 | A |
| IL-6 | C | AEV-1 | A |
| IM-8 | A | AEW-1 | A |
| AFP-3 | C | HT-3 | A |
| II-1 | C | HU-1 | C |
| IJ-1 | A | HM-3 | C |
| IK-1 | A | HN-1 | A |
| AFK-1 | A | HO-2 | A |
| AFL-2 | A | HP-2 | A |
| AFN-2 | A | HQ-2 | A |
| AFO-2 | A | HS-3 | A |
| AFM-1 | A | HG-7 | A |
| AFA-2 | B | HH-3 | A |
| IH-9 | A | HI-2 | A |
| AFB-2 | A | HJ-1 | A |
| AFJ-1 | A | HK-2 | A |
| ID-3 | A | HL-2 | A |
| AER-8 | A | AEP-2 | B |
| AFD-1 | A | AEN-15 | A |
| AFE-1 | A | AEY-2 | A |
| AEQ-2 | A | AEM-1 | A |
| AFC-1 | A | AEX-2 | B |

### Example 472: c-Cbl Biochemical Assay (TR-FRET)

Recombinant human c-Cbl (aa 47-435) was expressed in E coli, purified and biotinylated in vitro. The protein was diluted to 12 nM in freshly prepared assay buffer consisting of 50 mM HEPES, pH 7.0, 100 mM NaCl, 5 mM MgCl₂, 0.01% Triton-X 100, 0.01% BSA and 1 mM DTT. Recombinant human Src (aa 254-536)-GSSGSS-Zap-70 (aa 281-297) fusion protein was expressed in E coli and purified. Protein was diluted in assay buffer to 5-20 nM and ATP was added to 1 mM.

Recombinant human UBE2D2(C85K) was expressed in E coli, purified and ubiquitinated and Bodipy labelled in vitro. Protein was diluted to 200 nM in assay buffer without MgCl₂. (or Cisbio PPI buffer) Streptavidin-Terbium was added to 2 nM and EDTA to 10 mM, to provide a binding assay mix.

Compounds were dissolved in DMSO and diluted to prepare a ten-point half log dilution series. 100 nl of each compound concentration was dispensed in duplicate in a 384 well black assay plate using acoustic dispensing. Wells for maximum signal controls received 100 nl of DMSO only and wells for minimum controls received 100 nl of a reference inhibitor compound at a final assay concentration of 100 mM to produce 100% inhibition.

5 μl of diluted c-cbl enzyme was added to all wells of the assay plate and incubated at RT for 30 min. The enzyme assay was initiated by addition of 5 μl of Src-Zap/ATP mix to all wells, and the plate incubated at RT for 60-90 min. The enzyme reaction was terminated and the binding reaction was initiated by adding 10 μl of binding assay mix to all wells and incubating the plate at RT for 60 min prior to assay read.

Final assay conditions consisted of 6 nM c-cbl, 2.5-10 nM Src-Zap70, 0.5 mM ATP, 1% (v/v) DMSO (enzyme reaction) and 100 nM UBE2D2(C85K)-Ub-FL-BODIPY, 5 mM EDTA, 1 nM Streptavidin-Tb (binding reaction).

The HTRF assay signal was measured at 520 nm on an Envision plate reader, with reference signal at 485 or 620 nm. Data was normalized using maximum and minimum assay controls: % Inhibition=100−(100*((maximum control)−unknown)/(maximum control−minimum control)). A 4-parameter dose-response equation was used to fit the normalized dose-response data and derive an IC₅₀ for test compounds.

Table 3 lists the compounds of this invention tested in the TR-FRET hc-Cbl biochemical assay. Compounds having an activity designated as “A” provided an IC₅₀≤0.2 μM; compounds having an activity designated as “B” provided an IC₅₀>0.20 μM. but≤1 μM; compounds designated as “C” provided an IC₅₀>1 μM but≤10 μM; compounds designated as “D” provided an IC₅₀>10 μM but<50 μM.

TABLE 3
hc-Cbl Biochemical Assay (TR-FRET) Results

| Compound | TR-FRET hcCbl | Compound | TR-FRET hcCbl |
|---|---|---|---|
| ID | IC₅₀ (μM) | ID | IC₅₀ (μM) |
| AA-2 | C | ABB-2 | D |
| AAB-9 | C | ABC-4 | D |
| AAC-5 | D | ABD-9 | D |
| AAD-1 | C | ABH-4 | D |
| AAE-1 | A | ABJ-2 | D |
| AAG-4 | D | ABK-1 | C |
| AAI-5 | D | ABL-1 | D |
| AAJ-1 | D | ABO-1 | D |
| AAK-1 | D | ABR-2 | C |
| AAL-5 | C | ABS-2 | D |
| AAM-2 | D | ABT-2 | D |
| AAN-3 | D | ABU-2 | D |
| AAO-4 | D | ABV-1 | D |
| AAP-7 | C | ABX-3 | D |
| AAQ-2 | C | AC-2 | C |
| AAR-2 | C | ACA-2 | D |
| AAS-2 | C | ACB-1 | D |
| AAT-14 | D | ACC-1 | C |
| AAU-6 | D | ACF-1 | D |
| AAV-2 | D | ACG-3 | D |
| AAW-5 | C | ACJ-5 | D |
| AAX-12 | D | ACK-3 | D |
| AAY-2 | C | ACL-1 | C |
| AAZ-10 | C | ACM-1 | D |
| AB-2 | C | ACN-1 | B |
| ABA-1 | C | ACO-1 | D |
| ACQ-3 | C | AEB-1 | C |
| ACR-3 | D | AEC-2 | A |
| ACU-2 | D | AED-3 | D |
| ACZ-8 | A | AEE-1 | C |
| ADA-6 | A | AEF-2 | A |
| ADB-9 | A | AEG-8 | A |
| ADC-3 | D | AEH-2 | A |
| ADD-1 | C | AEI-1 | A |
| ADE-3 | A | AEJ-4 | C |
| ADF-2 | A | AEK-2 | C |
| ADG-1 | A | AEL-1 | B |
| ADH-1 | A | AD-2 | C |
| ADI-3 | A | AE-2 | C |
| ADJ-2 | C | AF-2 | C |
| ADK-1 | C | AG-2 | C |
| ADL-2 | A | AH-2 | C |
| ADM-14 | A | AI-2 | C |
| ADN-1 | B | AJ-3 | D |
| ADO-1 | A | AK-2 | C |
| ADP-1 | D | AL-2 | C |
| ADQ-1 | C | AM-2 | C |
| ADR-1 | A | AN-2 | C |
| ADS-1 | A | AO-2 | C |
| ADT-12 | A | AP-2 | C |
| ADU-2 | A | AQ-6 | C |
| ADV-2 | A | AR-2 | D |
| ADW-1 | A | AT-1 | D |
| ADX-3 | D | AU-1 | D |
| ADY-2 | A | AV-9 | D |
| ADZ-1 | A | AW-4 | D |
| AEA-1 | D | AX-3 | D |
| AY-5 | D | CK-2 | A |
| AZ-1 | D | CL-2 | B |
| BA-1 | D | CM-1 | A |
| BD-1 | D | CN-5 | B |
| BE-2 | D | CO-2 | C |
| BG-1 | D | CP-4 | A |
| BH-3 | D | CQ-1 | A |
| BI-1 | D | CR-2 | A |
| BJ-1 | C | CR-3 | A |
| BK-2 | C | CS-1 | A |
| BL-2 | C | CT-2 | B |
| BM-2 | C | CU-2 | A |
| BN-2 | C | CV-2 | A |
| BO-2 | C | CW-5 | A |
| BP-1 | C | CX-1 | A |
| BQ-1 | C | CY-1 | B |
| BR-1 | C | CZ-2 | A |
| BS-1 | C | DA-1 | A |
| BT-1 | C | DB-2 | C |
| BU-1 | C | DC-3 | D |
| BV-1 | C | DD-2 | B |
| BW-3 | D | DE-2 | A |
| BX-2 | D | DF-2 | A |
| BY-1 | C | DG-2 | A |
| BZ-1 | D | DH-2 | A |
| CB-1 | D | DI-1 | C |
| CC-2 | C | DJ-2 | B |
| CD-2 | C | DK-1 | C |
| CF-2 | D | DL-1 | C |
| CH-2 | D | DM-2 | A |
| CJ-1 | A | DN-2 | A |
| DO-6 | B | EO-2 | B |
| DO-7 | B | EP-2 | B |
| DP-5 | A | EQ-4 | B |
| DP-6 | A | ER-2 | A |
| DQ-1 | A | ES-4 | B |
| DR-2 | A | ES-6 | A |
| DS-2 | A | ET-8 | A |
| DT-2 | A | EU-2 | A |
| DT-3 | A | EV-9 | A |
| DU-2 | A | EW-2 | A |
| DV-2 | A | EX-6 | B |
| DW-3 | C | EY-2 | A |
| DX-2 | A | EZ-3 | B |
| DY-1 | B | FA-1 | A |
| DZ-1 | B | FB-2 | A |
| EA-1 | A | FC-2 | A |
| EA-2 | A | FD-2 | A |
| EB-1 | B | FE-3 | C |
| EC-1 | B | FF-6 | B |
| ED-7 | A | FG-2 | A |
| EE-2 | A | FH-2 | C |
| EF-1 | A | FI-2 | A |
| EG-2 | B | FJ-2 | A |
| EG-3 | B | FK-2 | B |
| EH-1 | A | FL-2 | A |
| EI-4 | B | FM-2 | A |
| EJ-2 | A | FN-1 | A |
| EK-5 | C | FO-2 | A |
| EL-1 | A | FP-1 | B |
| EM-2 | A | FQ-1 | B |
| EN-2 | B | FR-1 | B |
| FS-2 | A | HA-3 | A |
| FT-1 | A | HB-1 | A |
| FU-1 | A | HC-2 | B |
| FV-2 | A | HD-2 | A |
| FV-3 | A | HE-4 | A |
| FW-3 | A | HF-1 | B |
| FX-2 | A | F-5 | D |
| FY-1 | B | G-1 | D |
| FZ-5 | D | H-1 | D |
| GA-1 | A | I-2 | D |
| GB-2 | A | J-1 | D |
| GC-2 | A | K-2 | D |
| GD-1 | B | L-4 | D |
| GE-2 | B | M-5 | D |
| GF-2 | A | N-1 | C |
| GG-2 | A | 0-4 | D |
| GH-1 | B | P-6 | C |
| GI-2 | B | Q-3 | C |
| GJ-2 | B | R-3 | C |
| GK-5 | B | S-1 | C |
| GL-2 | C | T-5 | C |
| GM-2 | C | U-1 | C |
| GN-5 | D | V-1 | C |
| GO-2 | A | W-2 | C |
| GP-2 | A | X-1 | C |
| GU-1 | A | X-2 | C |
| GV-1 | B | Y-3 | C |
| GW-2 | A | Z-2 | C |
| GX-1 | B | AEY-2 | A |
| GY-1 | C | AEM-1 | A |
| GZ-1 | B | AEX-2 | C |
| AEO-1 | A | AFG-1 | A |
| AEN-15 | B | AFH-2 | B |
| AEQ-2 | A | IA-1 | A |
| AFC-1 | A | IB-7 | B |
| AEP-2 | B | IC-1 | A |
| AER-8 | A | AET-1 | C |
| AFD-1 | A | ID-3 | A |
| AFE-1 | A | IE-1 | B |
| HG-7 | A | IF-2 | B |
| HH-3 | A | IG-6 | A |
| HI-2 | A | AFB-2 | A |
| HJ-1 | A | AFI-1 | A |
| HK-2 | A | AFJ-1 | A |
| HL-2 | A | IH-9 | A |
| HM-3 | C | AFK-1 | A |
| HN-1 | A | AFL-2 | B |
| HO-2 | A | AFN-2 | A |
| HP-2 | A | AFO-2 | A |
| HQ-2 | A | AFM-1 | B |
| HS-3 | A | AFA-2 | B |
| HT-3 | A | II-1 | B |
| AEU-2 | A | IJ-1 | A |
| AEV-1 | A | IK-1 | A |
| AEW-1 | A | AFP-3 | B |
| HV-3 | C | IL-6 | C |
| HW-2 | A | IM-8 | A |
| HX-8 | A | AFQ-6 | A |
| HY-3 | A | AFR-1 | A |
| HZ-2 | B | AFS-2 | A |
| AEZ-1 | A | AFT-1 | B |
| AFF-1 | A | IN-2 | C |
| IO-4 | C | JC-1 | C |
| AFU-1 | A | JD-1 | A |
| AFV-1 | B | JE-1 | B |
| AFW-1 | C | JF-1 | B |
| IP-7 | A | AGM-8 | C |
| IQ-8 | A | AGN-3 | B |
| AFY-2 | A | JG-1 | A |
| AFX-1 | B | JH-1 | A |
| AFZ-2 | B | AGO-2 | B |
| IR-1 | B | JI-3 | A |
| AGA-1 | A | JJ-3 | A |
| AGB-1 | A | JK-7 | B |
| AGC-2 | A | AGQ-2 | A |
| IS-1 | A | AGP-7 | C |
| IT-1 | A | AGR-9 | C |
| AGD-1 | B | AGS-4 | A |
| AGE-1 | A | AGT-1 | A |
| AGF-1 | A | JL-1 | A |
| IU-2 | A | JM-4 | A |
| IV-2 | B | JN-1 | A |
| IW-1 | A | JO-1 | A |
| IX-1 | A | JP-1 | A |
| IY-1 | A | JQ-1 | A |
| IZ-1 | A | AGU-1 | A |
| AGG-2 | B | JR-2 | B |
| AGI-1 | A | JS-1 | A |
| AGH-4 | B | JT-1 | A |
| AGJ-1 | C | JU-1 | A |
| AGK-1 | A | AHC-1 | C |
| AGL-1 | B | AHD-1 | C |
| JB-2 | C | AHA-2 | B |
| AGZ-2 | B | AHH-3 | B |
| AHB-1 | A | AHI-5 | A |
| AHE-2 | A | AHJ-1 | A |
| AGX-1 | A | AHK-4 | A |
| AGY-1 | A | AHL-7 | C |
| AHF-1 | A | AHM-3 | B |
| AHG-1 | A | AHN-1 | A |

### Example 473: Jurkat Reporter Assay (NFAT Luciferase)

A commercial NFAT luciferase construct was purchased, and transfected into Jurkat cells to generate the stable cell line. Jurkat (clone E6-1) cells were engineered for stable expression of an NFAT luciferase reporter. Cells were maintained in RPMI 1640 medium with 10% FBS, 200 μg/ml Hygromycin B and 1% Penicillin/Streptomycin, and assayed in the same medium omitting Hygromycin B.

Test compounds were dissolved in DMSO (typically at 20 mM) and a ten-point half log dilution series prepared using acoustic dispensing. 125 nl of each compound concentration was dispensed in duplicate into wells of a 384 well white assay plate, typically providing a top final assay compound concentration of 100 mM. Assay low control wells received DMSO only and high controls received a standard compound (Example 183a in WO 2019/148005) providing a final assay concentration of 100 mM.

Jurkat cells were harvested and resuspended in assay medium at 4.445×10⁵ cells/ml. 22.5 μl (10,000 cells) was added to the wells of a white 384 well assay plate and incubated for 15 min at 37° C. in 5% CO₂. Anti-CD3 antibody (Thermofisher #16-0037-85) was diluted in assay medium to 10 μg/ml. 2.5 μl was added to wells of the assay plate and incubated for 6 hr at 37° C. in 5% CO₂. Additional control wells were included which received assay medium in place of anti-CD3.

Assay plates were equilibrated to RT and 25 μl of Steady-Glo® reagent (Promega #E2520) added to all wells. The plate was centrifuged at 100×g for 1 min and incubated for 10 min. Luminescence was read on an Envision plate reader.

Data was normalised using high and low assay controls: % activation=100−(100*((high control)−unknown)/(high control−low control)) Normalised data was fitted to a non-linear regression equation (log inhibitor vs response-variable slope 4-parameters) to derive compound EC₅₀ values. Fold activation over baseline was expressed as mean luminescence values from high control wells/mean luminescence values from low control wells (anti CD3 only). Maximum activation of compounds was also expressed as a normalised value referenced to the standard compound (maximum 00 activation of test compound/100)

Table 4 shows selected compounds of this invention tested in the Jurkat reporter assay. Compounds having an activity designated as “A” provided an EC₅₀≤0.2 μM; compounds having an activity designated as “B” provided an EC₅₀>0.20 μM. but<1 μM; compounds designated as “C” provided an EC₅₀>1 M but<10 μM; compounds designated as “D” provided an EC₅₀>10 μM but<50 μM.

TABLE 4
Jurkat reporter assay (NFAT Luciferase) Results

| Compound | Jurkat NFAT | Compound | Jurkat NFAT |
|---|---|---|---|
| ID | EC₅₀ (μM) | ID | EC₅₀ (μM) |
| AA-2 | B | ADA-6 | A |
| AAB-9 | B | ADB-9 | A |
| AAD-1 | B | ADE-3 | A |
| AAE-1 | B | ADF-2 | A |
| AAG-4 | C | ADG-1 | A |
| AAI-5 | C | ADH-1 | A |
| AAL-5 | C | ADI-3 | A |
| AAP-7 | B | ADK-1 | D |
| AAQ-2 | C | ADL-2 | A |
| AAR-2 | C | ADM-14 | A |
| AAS-2 | C | ADN-1 | B |
| AAW-5 | B | ADO-1 | A |
| AAX-12 | C | ADP-1 | D |
| AAY-2 | C | ADQ-1 | D |
| AAZ-10 | B | ADR-1 | A |
| AB-2 | C | ADS-1 | A |
| ABA-1 | B | ADT-12 | A |
| ABK-1 | C | ADU-2 | B |
| AC-2 | B | ADV-2 | A |
| ACF-1 | C | ADW-1 | A |
| ACK-3 | C | ADY-2 | A |
| ACM-1 | C | ADZ-1 | B |
| ACN-1 | C | AEC-2 | B |
| ACZ-8 | A | AEF-2 | B |
| AEG-8 | A | BY-1 | C |
| AEH-2 | A | CC-2 | C |
| AEI-1 | B | CD-2 | B |
| AEK-2 | C | CJ-1 | A |
| AEL-1 | D | CK-2 | A |
| AD-2 | C | CL-2 | C |
| AE-2 | B | CM-1 | A |
| AF-2 | B | CN-5 | B |
| AG-2 | C | CP-4 | A |
| AH-2 | B | CQ-1 | A |
| AI-2 | B | CR-2 | B |
| AK-2 | B | CR-3 | A |
| AL-2 | B | CS-1 | B |
| AM-2 | B | CT-2 | B |
| AN-2 | C | CU-2 | B |
| AO-2 | B | CV-2 | B |
| AP-2 | B | CW-5 | A |
| AQ-6 | B | CX-1 | B |
| AV-9 | C | CY-1 | C |
| BJ-1 | C | CZ-2 | B |
| BK-2 | C | DA-1 | A |
| BL-2 | C | DD-2 | C |
| BM-2 | B | DE-2 | B |
| BN-2 | B | DF-2 | A |
| BO-2 | B | DG-2 | A |
| BP-1 | B | DH-2 | A |
| BQ-1 | B | DI-1 | D |
| BR-1 | C | DJ-2 | C |
| BT-1 | B | DM-2 | A |
| BU-1 | B | DN-2 | B |
| BV-1 | B | DO-6 | C |
| DO-7 | C | EP-2 | B |
| DP-5 | B | EQ-4 | C |
| DP-6 | A | ER-2 | A |
| DQ-1 | A | ES-4 | C |
| DR-2 | B | ES-6 | A |
| DS-2 | A | ET-8 | B |
| DT-2 | A | EU-2 | A |
| DT-3 | A | EV-9 | A |
| DU-2 | A | EW-2 | A |
| DV-2 | A | EX-6 | C |
| DW-3 | D | EY-2 | A |
| DX-2 | A | EZ-3 | C |
| DY-1 | B | FA-1 | A |
| DZ-1 | C | FB-2 | A |
| EA-1 | A | FC-2 | B |
| EA-2 | A | FD-2 | B |
| EB-1 | C | FE-3 | D |
| EC-1 | C | FF-6 | C |
| ED-7 | A | FG-2 | A |
| EE-2 | A | FH-2 | D |
| EF-1 | A | FI-2 | A |
| EG-2 | C | FJ-2 | A |
| EG-3 | C | FK-2 | C |
| EH-1 | A | FL-2 | A |
| EI-4 | B | FM-2 | A |
| EJ-2 | A | FN-1 | A |
| EK-5 | C | FO-2 | A |
| EL-1 | B | FP-1 | B |
| EM-2 | A | FQ-1 | C |
| EN-2 | B | FR-1 | B |
| EO-2 | C | FS-2 | A |
| FT-1 | A | HB-1 | A |
| FU-1 | B | HC-2 | C |
| FV-2 | A | HD-2 | B |
| FV-3 | B | HE-4 | C |
| FW-3 | A | HF-1 | C |
| FX-2 | A | N-1 | C |
| FY-1 | C | P-6 | C |
| FZ-5 | D | Q-3 | C |
| GA-1 | A | R-3 | B |
| GB-2 | A | T-5 | B |
| GC-2 | A | U-1 | B |
| GD-1 | B | V-1 | C |
| GE-2 | C | W-2 | B |
| GF-2 | A | X-2 | C |
| GG-2 | B | Y-3 | B |
| GH-1 | B | Z-2 | C |
| GI-2 | C | AFY-2 | A |
| GJ-2 | C | AGB-1 | A |
| GK-5 | C | AHL-7 | C |
| GL-2 | D | AHM-3 | C |
| GM-2 | D | AHH-3 | C |
| GN-5 | D | AHI-5 | A |
| GO-2 | B | AHJ-1 | A |
| GP-2 | B | AHK-4 | A |
| GU-1 | A | JO-1 | A |
| GV-1 | B | AHF-1 | A |
| GW-2 | A | AHG-1 | A |
| GX-1 | C | AGF-1 | A |
| GY-1 | D | AHC-1 | C |
| GZ-1 | B | AHD-1 | C |
| HA-3 | A | AHA-2 | C |
| AGZ-2 | B | JH-1 | A |
| AHB-1 | A | AGM-8 | C |
| AHE-2 | A | AGN-3 | C |
| AGX-1 | A | JB-2 | C |
| AGY-1 | A | JC-1 | C |
| IT-1 | A | JD-1 | A |
| JR-2 | B | JE-1 | A |
| JS-1 | A | JF-1 | A |
| JT-1 | A | AGG-2 | B |
| JU-1 | A | AGI-1 | A |
| AGU-1 | A | AGH-4 | C |
| JL-1 | A | AGJ-1 | B |
| JM-4 | A | AGK-1 | A |
| JN-1 | A | AGL-1 | B |
| JP-1 | A | IZ-1 | A |
| JQ-1 | A | IY-1 | A |
| AGS-4 | A | IU-2 | B |
| AGT-1 | A | IV-2 | B |
| AFI-1 | A | IW-1 | A |
| AFQ-6 | A | IX-1 | A |
| AFR-1 | A | AGD-1 | C |
| AGP-7 | C | AGE-1 | A |
| AGR-9 | C | IS-1 | A |
| AEO-1 | A | AGA-1 | A |
| AGQ-2 | A | AGC-2 | A |
| JI-3 | A | IR-1 | B |
| JJ-3 | B | AFX-1 | B |
| JK-7 | C | AFZ-2 | B |
| AEZ-1 | A | IP-7 | A |
| AGO-2 | B | IQ-8 | A |
| JG-1 | A | AEU-2 | A |
| AFF-1 | A | IB-7 | C |
| AFU-1 | A | IC-1 | B |
| AFV-1 | B | AFG-1 | A |
| AFW-1 | C | AFH-2 | B |
| DB-1 | C | HV-3 | D |
| IN-2 | C | HW-2 | B |
| IO-4 | C | HX-8 | A |
| AFS-2 | B | HY-3 | B |
| AFT-1 | C | HZ-2 | C |
| IL-6 | B | AEV-1 | A |
| IM-8 | A | AEW-1 | A |
| AFP-3 | C | HT-3 | B |
| II-1 | C | HU-1 | D |
| IJ-1 | A | HM-3 | C |
| IK-1 | A | HN-1 | B |
| AFK-1 | B | HO-2 | A |
| AFL-2 | C | HP-2 | A |
| AFN-2 | B | HQ-2 | B |
| AFO-2 | B | HS-3 | A |
| AFM-1 | C | HG-7 | A |
| AFA-2 | C | HH-3 | A |
| IH-9 | B | HI-2 | A |
| AFB-2 | A | HJ-1 | A |
| AFJ-1 | B | HK-2 | A |
| ID-3 | A | HL-2 | B |
| IE-1 | B | AER-8 | A |
| IF-2 | C | AFD-1 | A |
| IG-6 | B | AFE-1 | A |
| AET-1 | C | AEQ-2 | A |
| AES-3 | D | AFC-1 | A |
| IA-1 | A | AEP-2 | C |
| AEN-15 | B | AEM-1 | A |
| AEY-2 | A | AEX-2 | C |

### Example 474: T-cell AlphaLISA Assay

Test compounds were dissolved in DMSO (typically at 20 mM) and a ten-point half log dilution series prepared using acoustic dispensing. 300 nl of each compound concentration was dispensed in duplicate into wells of a 384 well white assay plate, typically providing a top final assay compound concentration of 50 mM. Assay low control wells received DMSO only and high controls received a standard compound (Example 575 in WO2019/148005) providing a final assay concentration of 50 mM.

Cryopreserved human T-cells were thawed and incubated for 24 hrs prior to use in RPMI 1640 medium containing HI FBS 10% and 1% Penicillin-Streptomycin. Cells were harvested and resuspended in assay medium at a concentration of 1.25×10⁶ cells per ml. 40 μl (50,000 cells) were added to the wells of a white 384 well assay plate and 5 μl (0.5 μg/ml) anti-CD28 antibody (Life Technologies #16-0289-85) and 5 μL (0.75 μg/ml) anti-CD-3 antibody (Thermofisher #16-0037-85) is added to the appropriate wells. The plates were then incubated for 48 hours at 37° C. in 5% CO₂.

At the end of the incubation period, the assay plate was centrifuges at 300×g for 5 mins and 50 μL of the supernatant was transferred to a plate for IL-2 analysis. IL-2 levels were measured following the manufacturers protocol (Perkin Elmer #AL221C). Briefly, human IL-2 (0.3 μg) was dissolved in 100 μL 1× AlphaLISA Immunoassay buffer. A half log IL-2 standard curve was generated with a top concentration of 30 μg/mL in varying amounts of assay medium, as per manufacturers protocol. 2 μL of sample or IL-2 standard is added to 384-well Proxiplate alongside 8 μL of a 2.5× MIX of AlphaLISA Anti-Analyte Acceptor beads (10 μg/mL final) and Biotinylated Antibody Anti-Analyte (1 nM final). The plate is incubated for 60 minutes at 25° C. in the dark prior to the addition of 10 μL of 2× SA-Donor beads (40 μg/mL final) and further incubated for 30 minutes at 25° C. in the dark. Absorbance was read at 570 nm using Envision (PerkinElmer)

Data was normalised using high and low assay controls: % activation=100−(100*((high control)−unknown)/(high control−low control)). Normalised data was fitted to a non-linear regression equation (log inhibitor vs response-variable slope 4-parameters) to derive compound EC₅₀ values. Fold activation over baseline was expressed as mean luminescence values from high control wells/mean luminescence values from low control wells (anti CD3/anti-CD28 alone). Maximum activation of compounds was also expressed as a normalised value referenced to the standard compound (maximum % activation of test compound/100).

Table 5 shows the selected compounds of this invention tested in the T-cell assay. All compounds tested have an EC₅₀≤0.2 μM.

TABLE 5
T-cell AlphaLISA Assay Results

| Compound ID | Compound ID | Compound ID | Compound ID |
|---|---|---|---|
| AAB-9 | AV-9 | FJ-2 | FG-2 |
| AAD-1 | BM-2 | FT-1 | FJ-2 |
| AAE-1 | BN-2 | FW-3 | FT-1 |
| AAG-4 | BO-2 | HA-3 | FW-3 |
| AAI-5 | CM-1 | HB-1 | HA-3 |
| ABK-1 | CP-4 | CM-1 | HB-1 |
| ACM-1 | CR-3 | CP-4 | N-1 |
| ACZ-8 | DH-2 | CR-3 | P-6 |
| ADA-6 | DV-2 | DH-2 | Q-3 |
| ADB-9 | DX-2 | DV-2 | R-3 |
| ADE-3 | EA-2 | DX-2 | T-5 |
| ADI-3 | ED-7 | EA-2 | U-1 |
| ADL-2 | EH-1 | ED-7 | W-2 |
| ADM-14 | EJ-2 | EH-1 | Y-3 |
| ADO-1 | EM-2 | EJ-2 | AFY-2 |
| ADR-1 | ER-2 | EM-2 | AFI-1 |
| ADS-1 | ES-6 | ER-2 | AFQ-6 |
| ADV-2 | EU-2 | ES-6 | AGC-2 |
| ADW-1 | EV-9 | EU-2 | AEU-2 |
| AEH-2 | EW-2 | EV-9 | HG-7 |
| AA-2 | EY-2 | EW-2 | AEQ-2 |
| AC-2 | FA-1 | EY-2 | AEY-2 |
| AD-2 | FG-2 | FA-1 | |

### Example 475: Table 6. hCbl-b Displacement Assay Results

Table 6 shows select compounds of this invention tested in the hCbl-b Displacement Assay. All compounds tested have an IC₅₀≤10 μM.

TABLE 6
hCbl-b Displacement Assay Results

| Compound ID | Compound ID | Compound ID | Compound ID |
|---|---|---|---|
| AA-2 | BM-2 | HG-7 | JL-1 |
| AAW-5 | BN-2 | AEU-2 | JM-4 |
| AAZ-10 | BO-2 | AEV-1 | JO-1 |
| ABA-1 | BP-1 | HW-2 | JQ-1 |
| AC-2 | BT-1 | HX-8 | AGU-1 |
| AE-2 | BV-1 | AFF-1 | JS-1 |
| AG-2 | CD-2 | AFB-2 | JT-1 |
| AH-2 | R-3 | AFQ-6 | JU-1 |
| AI-2 | T-5 | AFR-1 | AGX-1 |
| AK-2 | U-1 | IP-7 | AGY-1 |
| AL-2 | W-2 | JG-1 | AHI-5 |
| AM-2 | Y-3 | JH-1 | |
| AP-2 | AEQ-2 | AGT-1 | |

While we have described a number of embodiments of this invention, it is apparent that our examples may be altered to provide other embodiments that utilize the compounds and methods of this invention. Therefore, it will be appreciated that the scope of this invention is to be defined by the appended claims rather than by the specific embodiments that have been represented by way of example.

1. A compound of formula I: 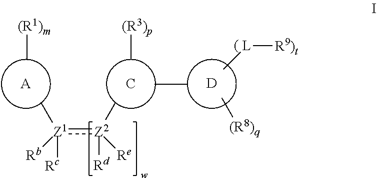
or a pharmaceutically acceptable salt thereof, wherein:
Ring A is a 5-6-membered heteroaryl ring having 1-4 heteroatoms independently selected from nitrogen, oxygen, and sulfur; or a 5-6-membered saturated or partially unsaturated heterocyclic ring having 1-4 heteroatoms independently selected from nitrogen, oxygen, and sulfur;
each R¹ is independently hydrogen, oxo, halogen, —CN, —NO₂, —CHF₂, —CF₃, —OR, —SR, —N(R)₂, —S(O)₂R, —S(O)₂N(R)₂, —S(O)R, —S(O)N(R)₂, —C(O)R, —C(O)OR, —C(O)N(R)₂, —C(O)N(R)OR, —OC(O)R, —OC(O)N(R)₂, —N(R)C(O)OR, —N(R)C(O)R, —N(R)C(O)N(R)₂, —N(R)C(NR)N(R)₂, —N(R)N(R)₂, —N(R)S(O)₂N(R)₂, —N(R)S(O)₂R, —N═S(O)(R)₂, —S(NR)(O)R, —N(R)S(O)R, —N(R)CN, —P(O)(R)N(R)₂, —P(O)(R)OR, or —P(O)(R)₂; or an optionally substituted C₁₋₆ aliphatic;
Z¹ is C or N;
Z² is C or N;
each of R^{b}, R^{c}, R^{d}, and R^{e} is independently hydrogen, halogen, —CN, —NO₂, —CHF₂, —CF₃, —OR, —SR, —N(R)₂, —S(O)₂R, —S(O)₂N(R)₂, —S(O)R, —S(O)N(R)₂, —C(O)R, —C(O)OR, —C(O)N(R)₂, —C(O)N(R)OR, —OC(O)R, —OC(O)N(R)₂, —N(R)C(O)OR, —N(R)C(O)R, —N(R)C(O)N(R)₂, —N(R)C(NR)N(R)₂, —N(R)N(R)₂, —N(R)S(O)₂N(R)₂, —N(R)S(O)₂R, —N═S(O)(R)₂, —S(NR)(O)R, —N(R)S(O)R, —N(R)CN, —P(O)(R)N(R)₂, —P(O)(R)OR, or —P(O)(R)₂; or each of R^{b}, R^{c}, R^{d}, and R^{e} is independently an optionally substituted group selected from C₁₋₆ aliphatic; phenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, phosphorous, silicon and sulfur; and a 5-6 membered monocyclic heteroaryl ring having 1-4 heteroatoms independently selected from nitrogen, oxygen, and sulfur; wherein R^{c} and R^{e} are absent if not allowed by valence; or
R^{b} and R^{c}, together with Z¹, are taken together to form an optionally substituted group selected from a 3-7 membered saturated or partially unsaturated monocyclic carbocyclic ring; a 5-6 membered monocyclic aryl ring; a 3-7 membered saturated or partially unsaturated monocyclic heterocyclic ring having 1-2 heteroatoms independently selected from nitrogen, oxygen, phosphorous, silicon and sulfur; and a 5-6 membered monocyclic heteroaryl ring having 1-4 heteroatoms independently selected from nitrogen, oxygen, and sulfur; or
R^{d} and R^{e}, together with Z², are taken together to form an optionally substituted group selected from a 3-7 membered saturated or partially unsaturated monocyclic carbocyclic ring; a 5-6 membered monocyclic aryl ring; a 3-7 membered saturated or partially unsaturated monocyclic heterocyclic ring having 1-2 heteroatoms independently selected from nitrogen, oxygen, phosphorous, silicon and sulfur; and a 5-6 membered monocyclic heteroaryl ring having 1-4 heteroatoms independently selected from nitrogen, oxygen, and sulfur; or
R^{b} and R^{d} together with the atoms to which each are attached, forms 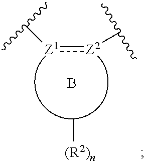
wherein Ring B is a divalent phenyl or a divalent 5-6 membered heteroaryl ring having 1-4 heteroatoms independently selected from nitrogen, oxygen, and sulfur;
each R² is independently hydrogen, oxo, halogen, —CN, —NO₂, —CHF₂, —CF₃, —OR, —CH₂OR, —OCH₂C≡CR, —CH₂N(R)₂, —SR, —N(R)₂, —S(O)₂R, —S(O)₂N(R)₂, —S(O)R, —S(O)N(R)₂, —C(O)R, —C(O)OR, —C(O)N(R)₂, —C(O)N(R)OR, —OC(O)R, —OC(O)N(R)₂, —N(R)C(O)OR, —N(R)C(O)R, —N(R)C(O)N(R)₂, —N(R)C(NR)N(R)₂, —N(R)N(R)₂, —N(R)S(O)₂N(R)₂, —N(R)S(O)₂R, —N═S(O)(R)₂, —S(NR)(O)R, —N(R)S(O)R, —N(R)CN, —P(O)(R)N(R)₂, —P(O)(R)OR, or —P(O)(R)₂; or an optionally substituted C₁₋₆ aliphatic;
Ring C is a divalent phenyl or a divalent 5-6 membered heteroaryl ring having 1-4 heteroatoms independently selected from nitrogen, oxygen, and sulfur;
each R³ is independently hydrogen, oxo, halogen, —CN, —NO₂, —CHF₂, —CF₃, —OR, —SR, —N(R)₂, —S(O)₂R, —S(O)₂N(R)₂, —S(O)R, —S(O)N(R)₂, —C(O)R, —C(O)OR, —C(O)N(R)₂, —C(O)N(R)OR, —OC(O)R, —OC(O)N(R)₂, —N(R)C(O)OR, —N(R)C(O)R, —N(R)C(O)N(R)₂, —N(R)C(NR)N(R)₂, —N(R)N(R)₂, —N(R)S(O)₂N(R)₂, —N(R)S(O)₂R, —N═S(O)(R)₂, —S(NR)(O)R, —N(R)S(O)R, —N(R)CN, —P(O)(R)N(R)₂, —P(O)(R)OR, or —P(O)(R)₂; or an optionally substituted group selected from C₁₋₆ aliphatic; a phenyl ring; a 3-7 membered saturated or partially unsaturated monocyclic carbocyclic ring; a 5-6 membered monocyclic heteroaryl ring having 1-4 heteroatoms independently selected from nitrogen, oxygen, and sulfur; and a 4-8 membered saturated or partially unsaturated monocyclic heterocyclic ring having 1-2 heteroatoms independently selected from nitrogen, oxygen, and sulfur;
or two R³ groups, and the atoms to which each R³ group is attached, are optionally taken together to form a fused 3-7 membered saturated or partially unsaturated monocyclic carbocyclic ring; a fused 5-6 membered monocyclic aryl ring; a fused 3-7 membered saturated or partially unsaturated monocyclic heterocyclic ring having 1-2 heteroatoms independently selected from nitrogen, oxygen, phosphorous, silicon and sulfur; or a fused 5-6 membered monocyclic heteroaryl ring having 1-4 heteroatoms independently selected from nitrogen, oxygen, and sulfur;
Ring D is a 3-7 membered saturated or partially unsaturated monocyclic carbocyclic ring; a 4-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; a 5-10 membered saturated or partially unsaturated bridged bicyclic ring having 0-5 heteroatoms selected from nitrogen, oxygen, and sulfur; a 6-10 membered saturated or partially unsaturated spiro bicyclic ring having 0-5 heteroatoms independently selected from nitrogen, oxygen, and sulfur; or a 6-10 membered saturated, partially unsaturated, or unsaturated fused bicyclic ring having 0-5 heteroatoms independently selected from nitrogen, oxygen, and sulfur;
each R⁸ is independently hydrogen, oxo, halogen, —CN, —NO₂, —CHF₂, —CH₂F, —CF₃, —OR, —SR, —SF₅, —N(R)₂, —S(O)₂R, —S(O)₂N(R)₂, —S(O)R, —S(O)N(R)₂, —C(O)R, —C(O)OR, —C(O)N(R)₂, —C(O)N(R)OR, —OC(O)R, —OC(O)N(R)₂, —N(R)C(O)OR, —N(R)C(O)R, —N(R)C(O)N(R)₂, —N(R)C(NR)N(R)₂, —N(R)N(R)₂, —N(R)S(O)₂N(R)₂, —N(R)S(O)₂R, —N═S(O)(R)₂, —S(NR)(O)R, —N(R)S(O)R, —N(R)CN, —P(O)(R)N(R)₂, —P(O)(R)OR, or —P(O)(R)₂; or an optionally substituted groups selected from C₁₋₆ aliphatic; phenyl; a 3-7 membered saturated or partially unsaturated monocyclic carbocyclic ring; a 4-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur;
L is a covalent bond; or L is a C₁₋₄ bivalent saturated or unsaturated, straight or branched hydrocarbon chain wherein one or two methylene units of the chain are optionally and independently replaced by —C(R)₂—, —N(R)—, —O—, —S—, —S(O)—, —S(O)₂—, —S(O)N(R)—, —N(R)S(O)—, —S(O)₂N(R)—, —N(R)S(O)₂—, —C(O)—, —C(O)O—, —OC(O)—, —C(O)N(R)—, —N(R)C(O)—, —C(O)N(R)O—, —ON(R)C(O)—, —OC(O)N(R)—, —N(R)C(O)O—, or —N(R)C(O)N(R)—;
R⁹ is halogen, —CN, —NO₂, —OR, —SR, —N(R)₂, —S(O)₂R, —S(O)₂N(R)₂, —S(O)R, —S(O)N(R)₂, —CF₂R, —CF₃, —C(R)₂OR, —C(R)₂N(R)₂, —C(O)R, —C(O)OR, —C(O)N(R)₂, —C(O)N(R)OR, —OC(O)R, —OC(O)N(R)₂, —C(S)N(R)₂, —N(R)C(O)OR, —N(R)C(O)R, —N(R)C(O)N(R)₂, —N(R)C(NR)N(R)₂, —N(R)N(R)₂, —N(R)S(O)₂N(R)₂, —N(R)S(O)₂R, —N═S(O)(R)₂, —S(NR)(O)R, —N(R)S(O)R, —N(R)CN, —Si(OR)(R)₂, —Si(R)₃, —P(O)(R)N(R)₂, —P(O)(R)OR, or —P(O)(R)₂; or R⁹ is an optionally substituted group selected from C₁₋₆ aliphatic; phenyl; a 3-7 membered saturated or partially unsaturated monocyclic carbocyclic ring; a 4-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; a 5-8 membered saturated or partially unsaturated bridged bicyclic ring having 0-3 heteroatoms selected from nitrogen, oxygen, and sulfur; a 6-10 membered saturated or partially unsaturated spiro bicyclic ring having 0-3 heteroatoms independently selected from nitrogen, oxygen, and sulfur; and an 8-10 membered partially aromatic or heteroaromatic bicyclic heterocyclic ring having 1-4 heteroatoms independently selected from nitrogen, oxygen, and sulfur;
each instance of R¹, R², R³, R⁸, and R⁹ is independently and optionally substituted with v instances of R^{A}, wherein each R^{A} is independently oxo, halogen, —CN, —NO₂, —OR, —SR, —N(R)₂, —S(O)₂R, —S(O)₂N(R)₂, —S(O)R, —S(O)N(R)₂, —C(R)₂OR, —C(O)R, —C(O)OR, —C(O)N(R)₂, —C(O)N(R)OR, —OC(O)R, —OC(O)N(R)₂, —N(R)C(O)OR, —N(R)C(O)R, —N(R)C(O)N(R)₂, —N(R)C(NR)N(R)₂, —N(R)N(R)₂, —N(R)S(O)₂N(R)₂, —N(R)S(O)₂R, —N═S(O)(R)₂, —S(NR)(O)R, —N(R)S(O)R, —N(R)CN, —P(O)(R)N(R)₂, —P(O)(R)OR, —P(O)(R)₂, or an optionally substituted group selected from C₁₋₆ aliphatic; phenyl; naphthyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, phosphorous, silicon and sulfur; a 5-6 membered monocyclic heteroaryl ring having 1-4 heteroatoms independently selected from nitrogen, oxygen, and sulfur; an 8-10 membered bicyclic heteroaryl ring having 1-5 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; and a 6-11 membered saturated or partially unsaturated bicyclic heterocyclic ring having 1-2 heteroatoms independently selected from nitrogen, oxygen, and sulfur;
each R is independently hydrogen, —CN, halogen, or an optionally substituted group selected from C₁₋₆ aliphatic; phenyl; naphthyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 7-12 membered saturated or partially unsaturated bicyclic heterocyclic ring having 1-4 heteroatoms independently selected from nitrogen, oxygen, and sulfur; a 5-8 membered saturated or partially unsaturated bridged bicyclic ring having 0-3 heteroatoms independently selected from nitrogen, oxygen, and sulfur; a 6-10 membered saturated or partially unsaturated spirocyclic ring having 0-3 heteroatoms independently selected from nitrogen, oxygen, and sulfur; a 6-11 membered saturated or partially unsaturated bicyclic carbocyclic ring having 1-2 heteroatoms independently selected from nitrogen, oxygen, and sulfur; or:
two R groups on the same atom are optionally taken together with the atom to form an optionally substituted 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur;
m is 0, 1, 2, 3, 4, or 5;
n is 0, 1, 2, 3, or 4;
p is 0, 1, 2, 3, or 4;
q is 0, 1, 2, or 3;
t is 0 or 1;
each instance of v is independently 0, 1, 2, 3, 4, or 5;
w is 0 or 1; and
wherein [symbol] denotes a single or double bond.

2-3. (canceled)

4. The compound of claim 1, wherein Ring A is selected from

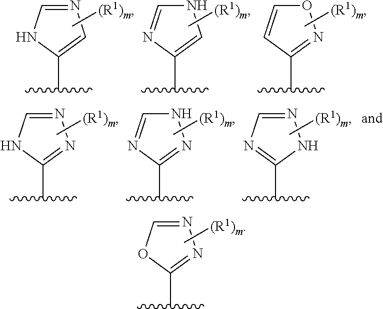

5. The compound of claim 4, wherein R¹ is halogen, —CN, —NO₂, —CHF₂, —CF₃, —OR, —N(R)₂, —C(O)R, —C(O)OR, —C(O)N(R)₂, —OC(O)R, —OC(O)N(R)₂, —N(R)C(O)OR, —N(R)C(O)R, —N(R)C(O)N(R)₂, —N(R)S(O)₂R, or —N(R)S(O)R; or an optionally substituted -Me, -Et, —Pr, -i-Pr, -n-Bu, -s-Bu, -t-Bu, straight chain or branched pentyl, or straight chain or branched hexyl.

6. The compound of claim 1, wherein each of R^{b}, R^{c}, R^{d}, and R^{e} is independently hydrogen, halogen, —CN, —NO₂, —CHF₂, —CF₃, —OR, —SR, —N(R)₂, —S(O)₂R, —S(O)₂N(R)₂, —S(O)R, —S(O)N(R)₂, —C(O)R, —C(O)OR, —C(O)N(R)₂, —C(O)N(R)OR, —OC(O)R, —OC(O)N(R)₂, —N(R)C(O)OR, —N(R)C(O)R, —N(R)C(O)N(R)₂, —N(R)C(NR)N(R)₂, —N(R)N(R)₂, —N(R)S(O)₂N(R)₂, —N(R)S(O)₂R, —N═S(O)(R)₂, —S(NR)(O)R, —N(R)S(O)R, —N(R)CN, —P(O)(R)N(R)₂, —P(O)(R)OR, or —P(O)(R)₂; or each of R^{b}, R^{c}, R^{d}, and R^{e} is independently an optionally substituted group selected from C₁₋₆ aliphatic; phenyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, phosphorous, silicon and sulfur; and a 5-6 membered monocyclic heteroaryl ring having 1-4 heteroatoms independently selected from nitrogen, oxygen, and sulfur; wherein R^{c} and R^{e} are absent if not allowed by valence.

7. The compound of claim 1, wherein R^{b} and R^{c}, together with Z¹, are taken together to form an optionally substituted group selected from a 3-7 membered saturated or partially unsaturated monocyclic carbocyclic ring; a 5-6 membered monocyclic aryl ring; a 3-7 membered saturated or partially unsaturated monocyclic heterocyclic ring having 1-2 heteroatoms independently selected from nitrogen, oxygen, phosphorous, silicon and sulfur; and a 5-6 membered monocyclic heteroaryl ring having 1-4 heteroatoms independently selected from nitrogen, oxygen, and sulfur.

8. The compound of claim 1, wherein R^{d}, and R^{e}, together with Z², are taken together to form an optionally substituted group selected from a 3-7 membered saturated or partially unsaturated monocyclic carbocyclic ring; a 5-6 membered monocyclic aryl ring; a 3-7 membered saturated or partially unsaturated monocyclic heterocyclic ring having 1-2 heteroatoms independently selected from nitrogen, oxygen, phosphorous, silicon and sulfur; and a 5-6 membered monocyclic heteroaryl ring having 1-4 heteroatoms independently selected from nitrogen, oxygen, and sulfur.

9. The compound of claim 1, wherein the group 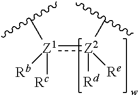
is

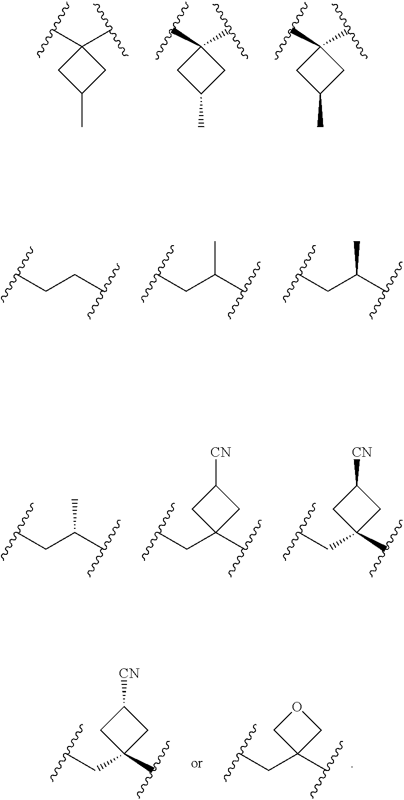

10-11. (canceled)

12. The compound of claim 1, wherein Ring B is a divalent group selected from

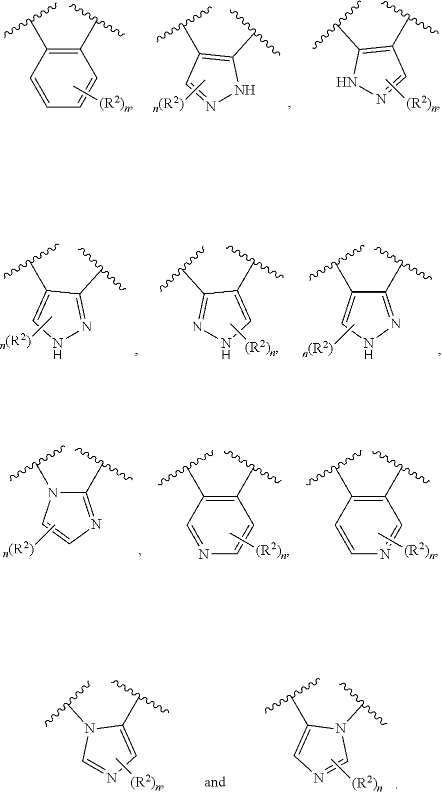

13. The compound of claim 12, wherein Ring B together with its R² substituents is

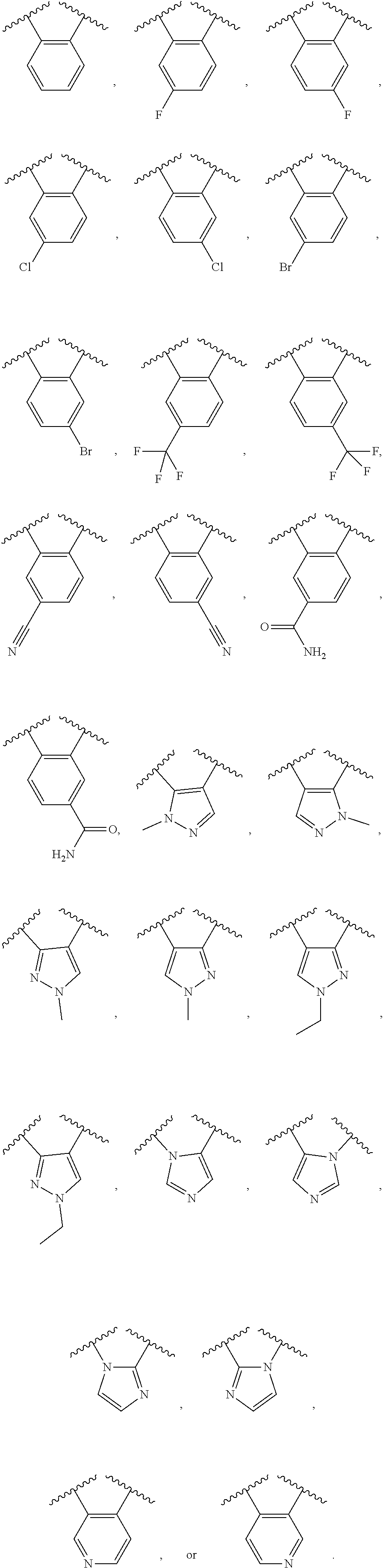

14. The compound of claim 12, wherein R² is halogen, —CN, —NO₂, —CHF₂, —CF₃, —OR, —SR, —N(R)₂, —S(O)₂R, —S(O)₂N(R)₂, —S(O)R, —S(O)N(R)₂, —C(O)R, —C(O)OR, —C(O)N(R)₂, —OC(O)R, —OC(O)N(R)₂, —N(R)C(O)OR, —N(R)C(O)R, —N(R)C(O)N(R)₂, —N(R)S(O)₂R, —N(R)S(O)R, or an optionally substituted C₁₋₆ aliphatic.

15. (canceled)

16. The compound of claim 1, wherein Ring C is

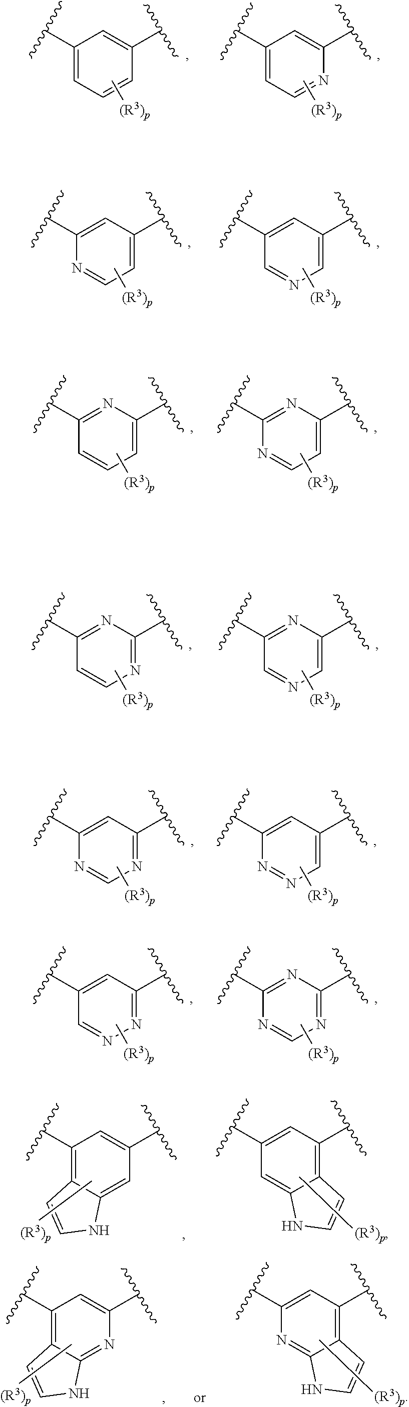

17. The compound of claim 16, wherein each R³ is independently —H, halogen, —OR, —NR₂, —C(O)R, —C(O)OR, —C(O)NR₂, —C(O)N(R)OR, —N(R)C(O)OR, —N(R)C(O)R, —N(R)C(O)NR₂, —N(R)S(O)₂R, or —N(R)S(O)R; or each instance of R³ is independently an optionally substituted C₁₋₆ aliphatic; or a 3-7 membered saturated or partially unsaturated monocyclic carbocyclic ring.

18. The compound of claim 1, wherein Ring D is selected from

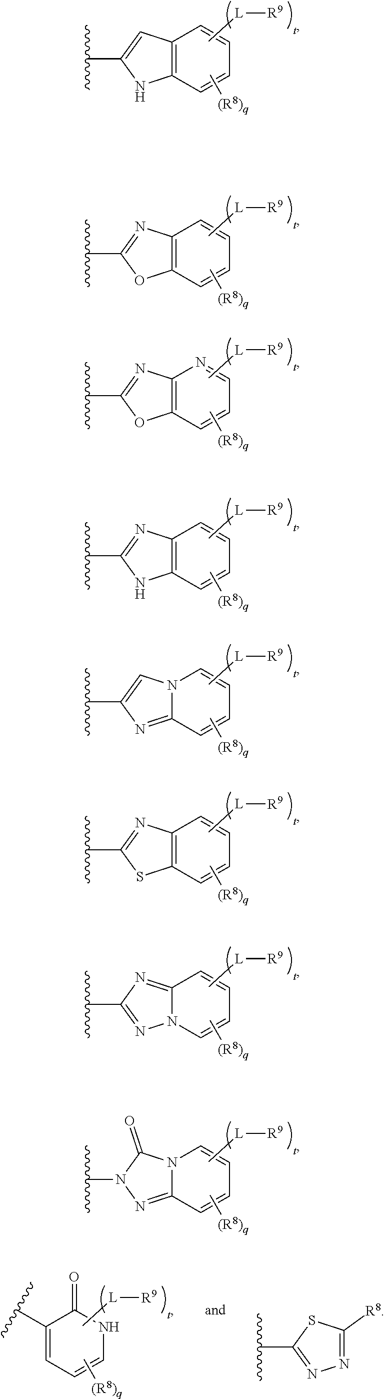

19. The compound of claim 18, wherein -L-R⁹ is

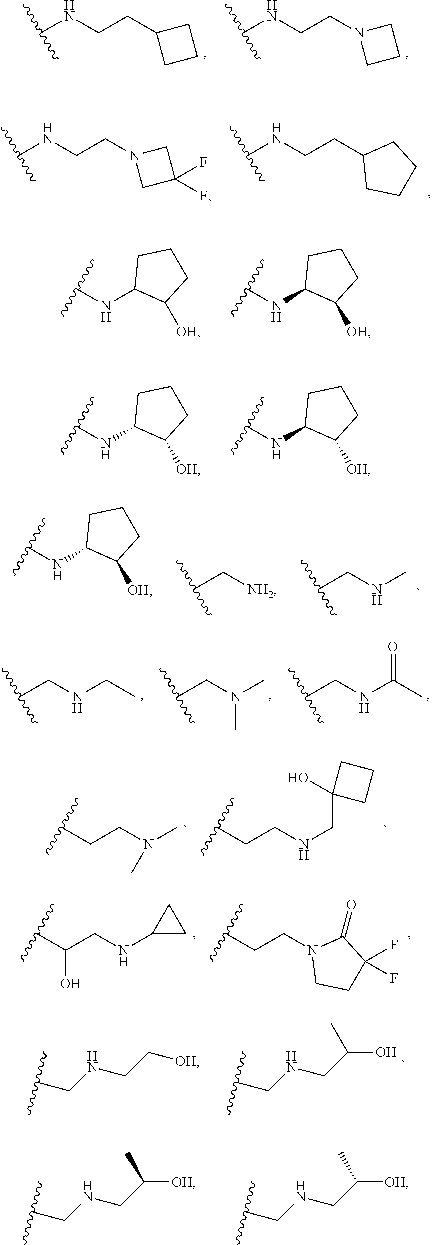

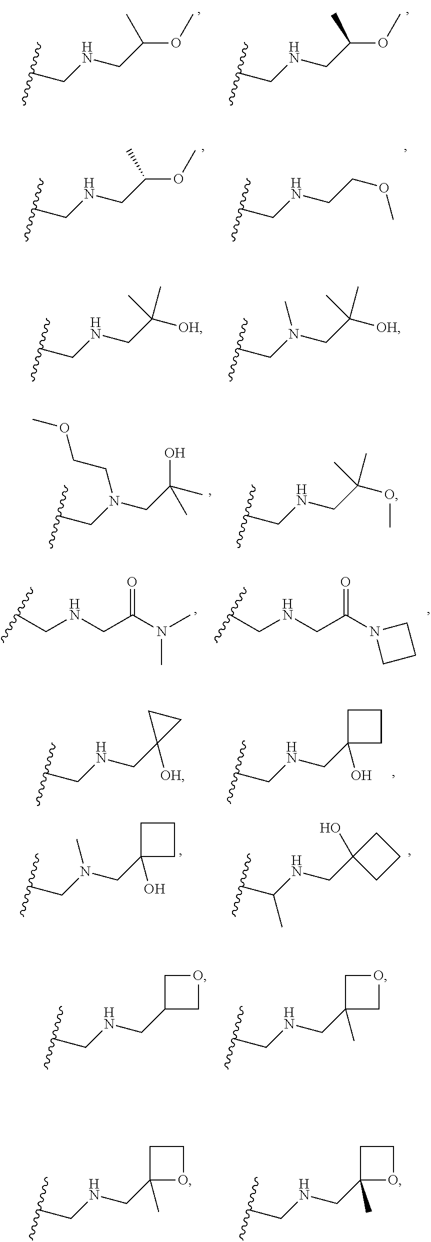

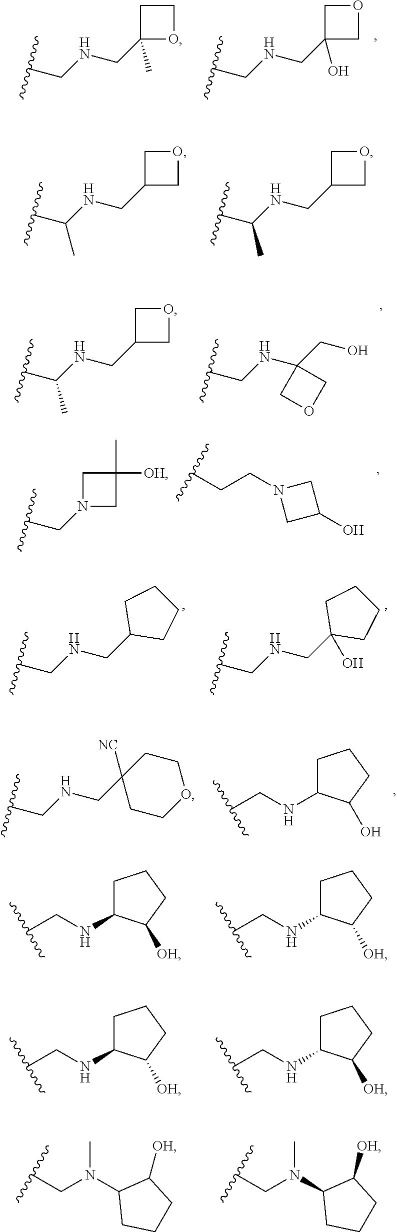

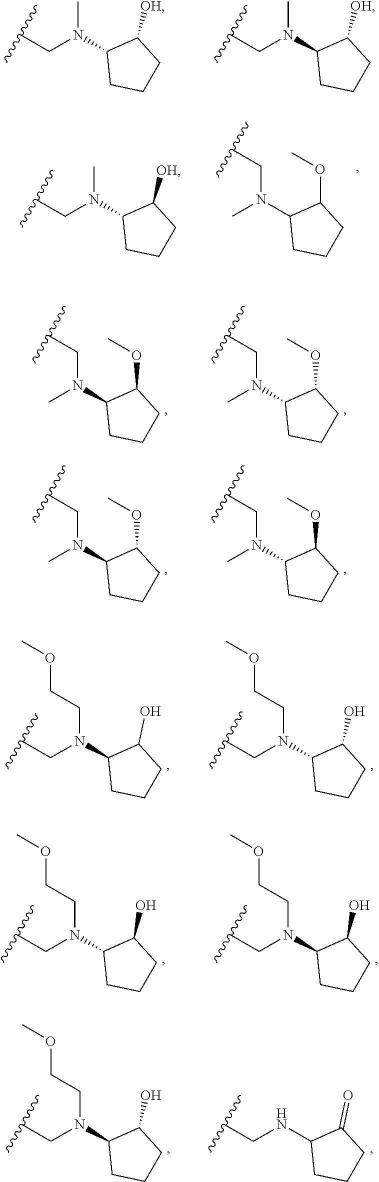

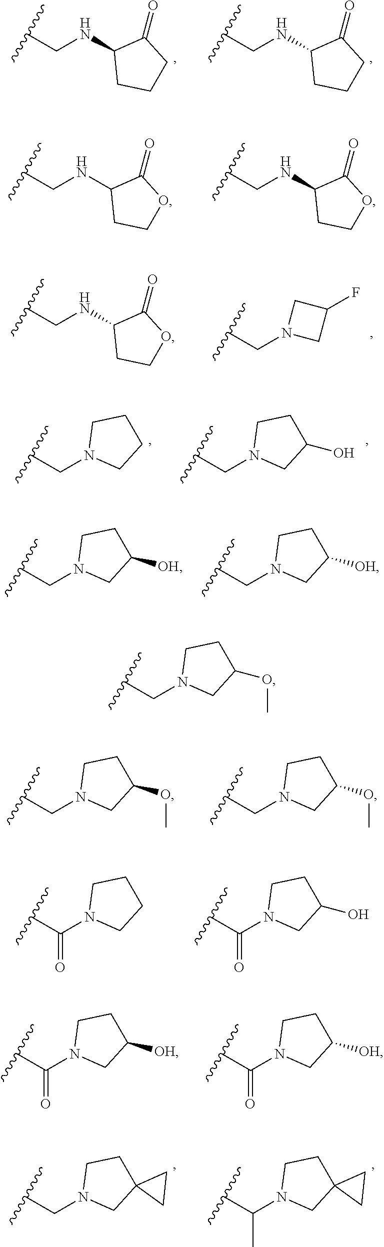

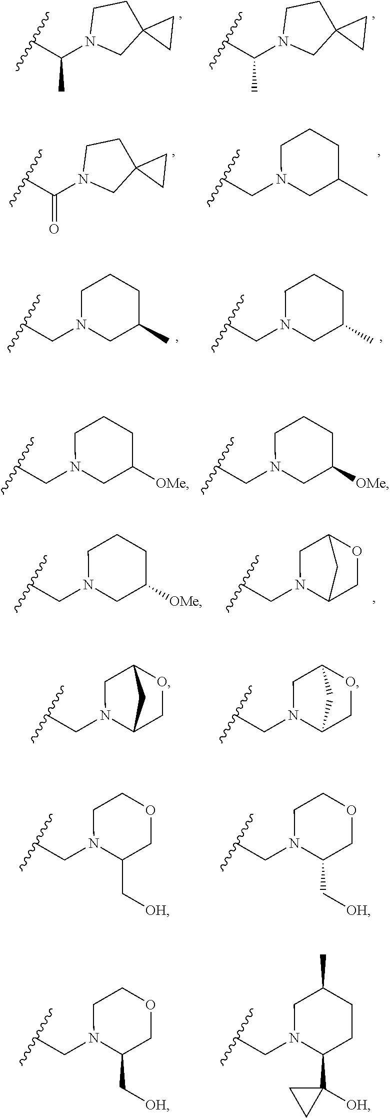

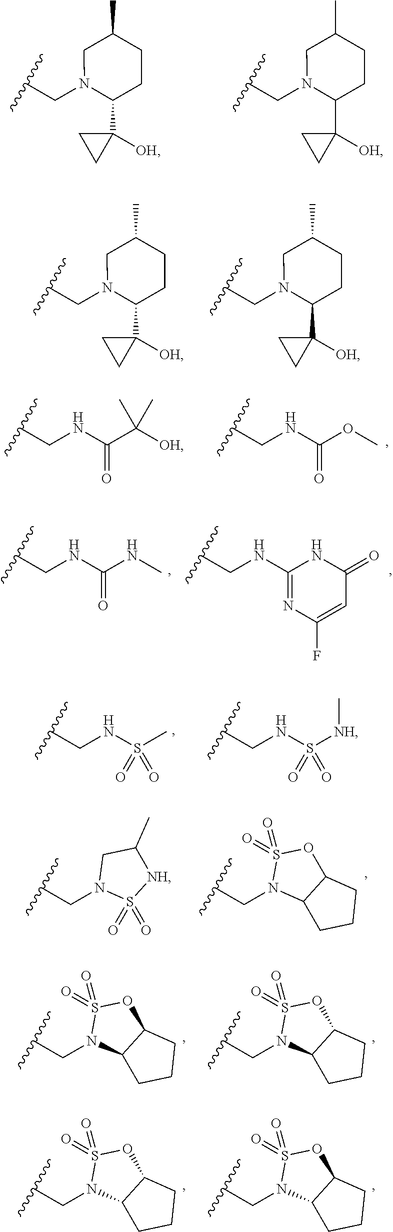

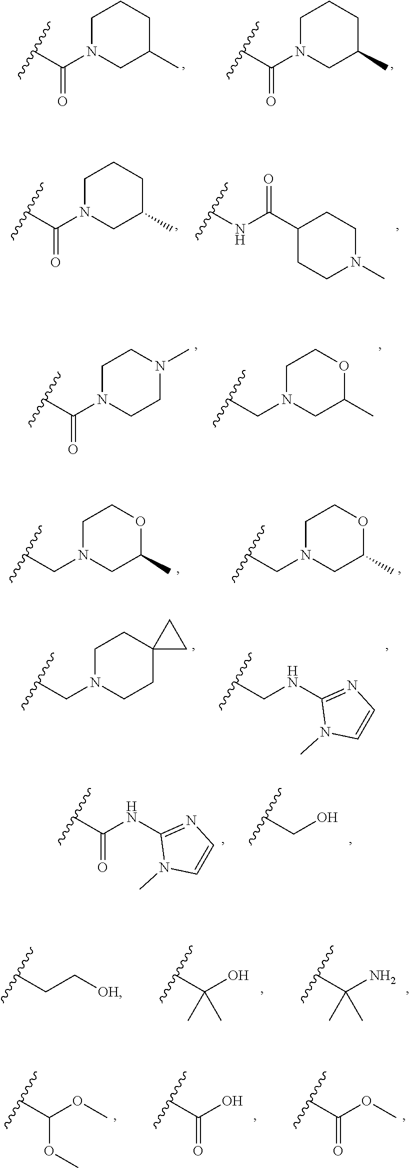

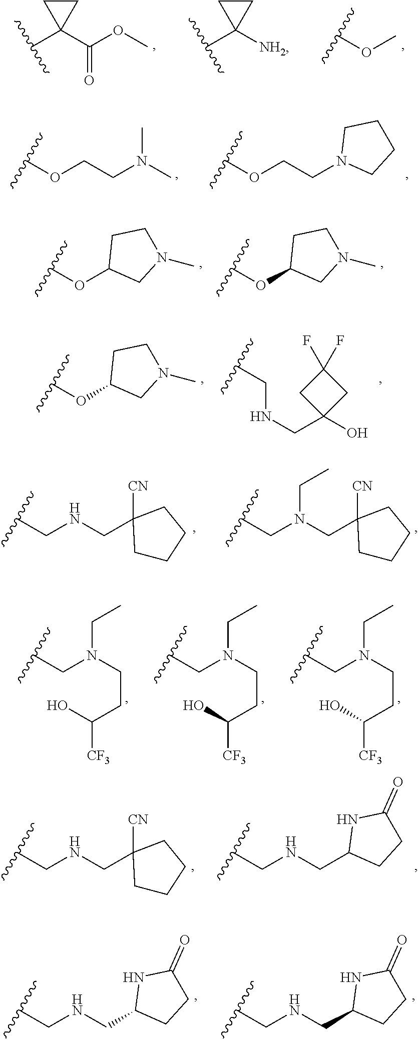

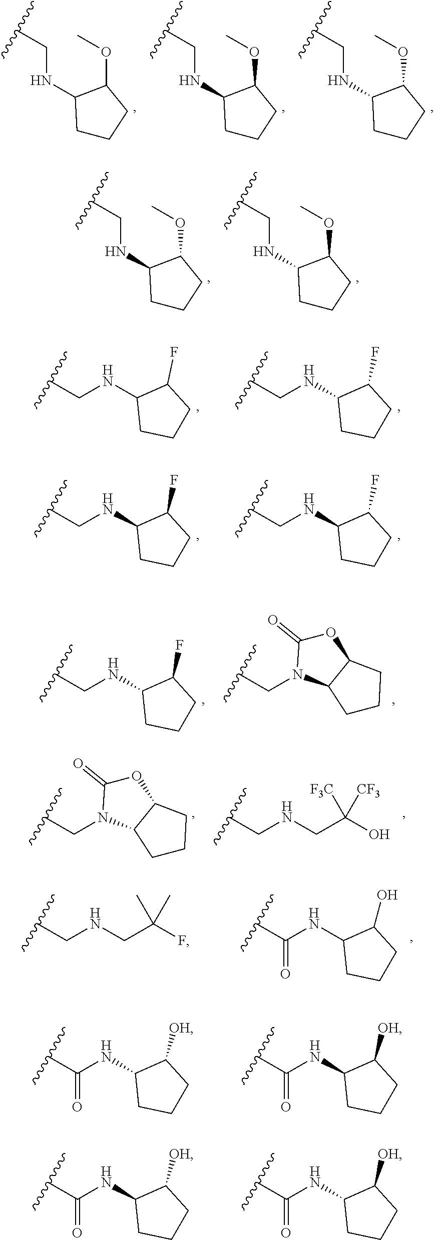

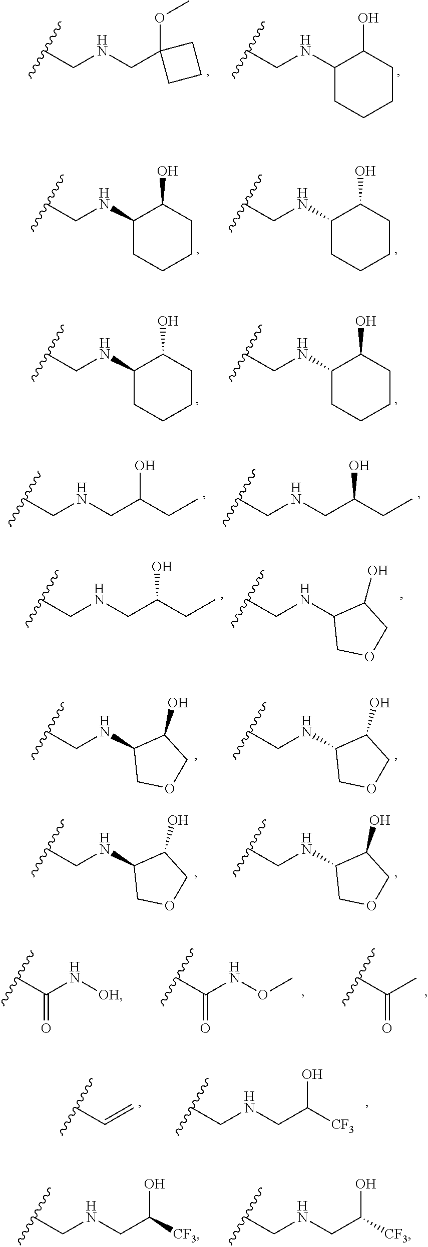

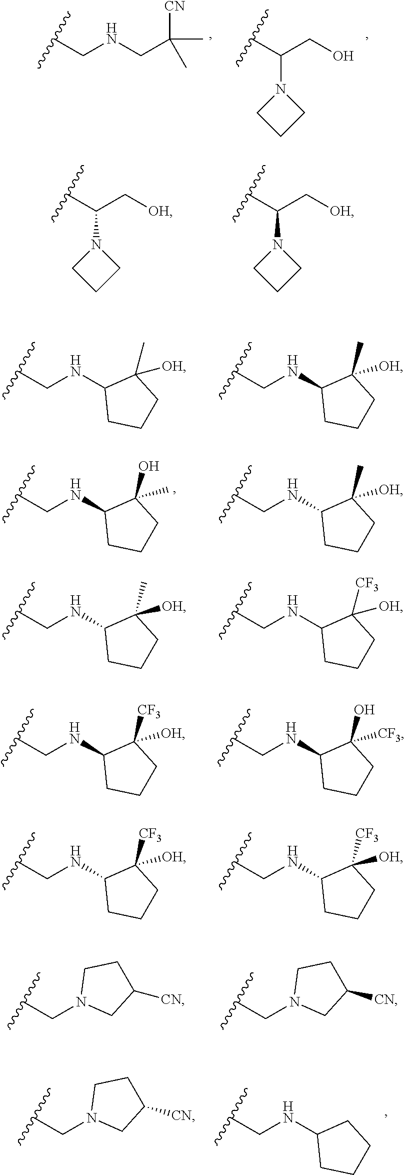

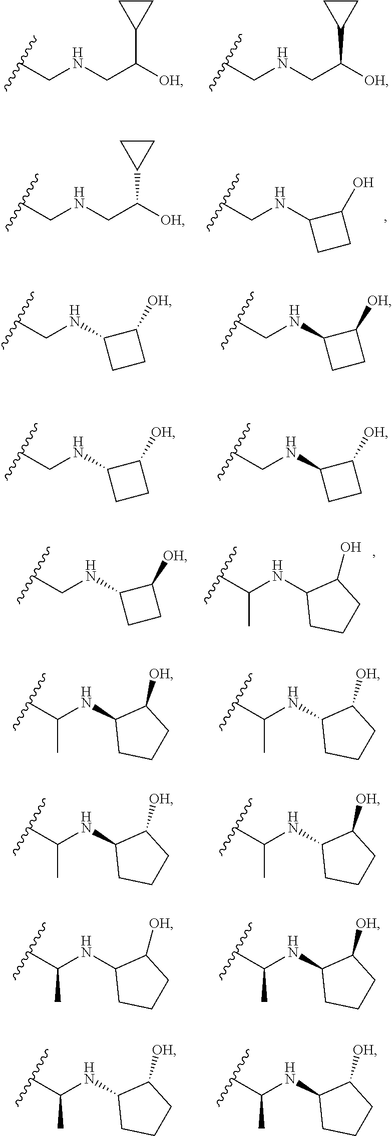

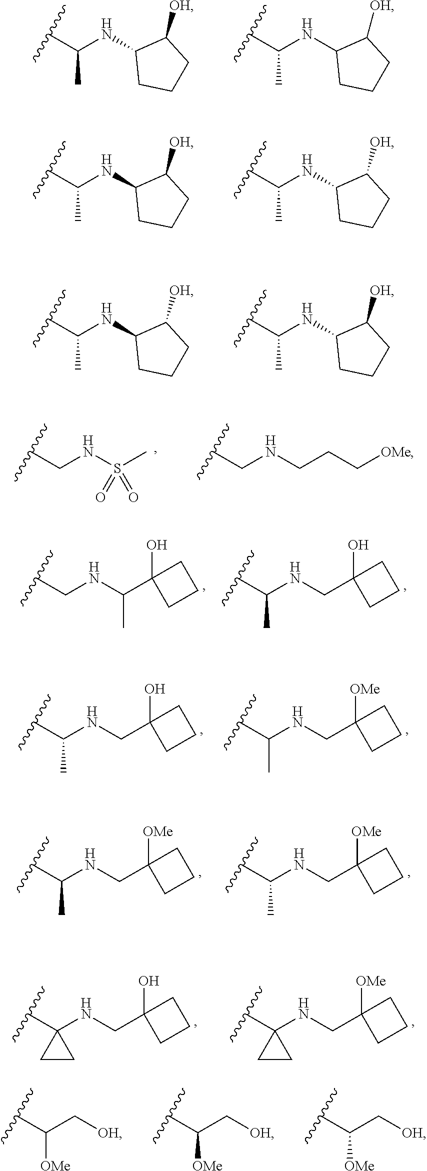

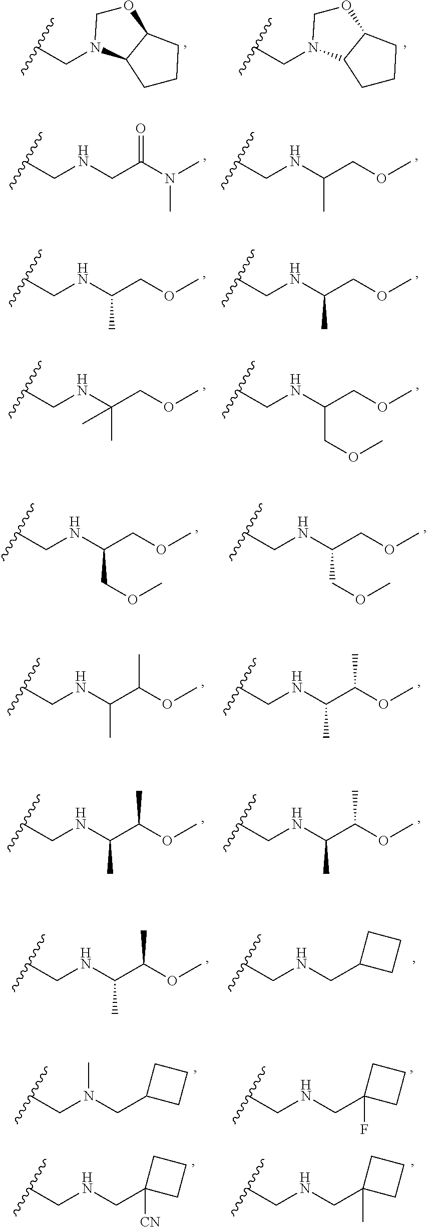

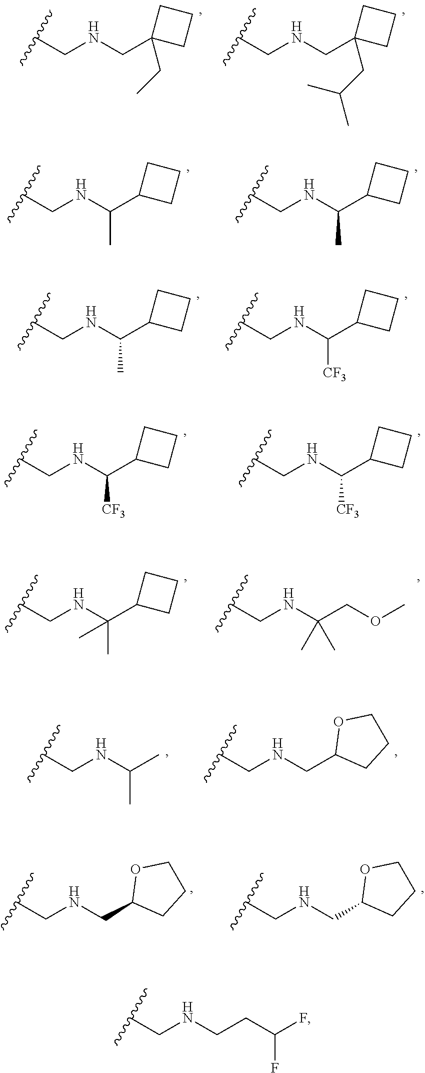

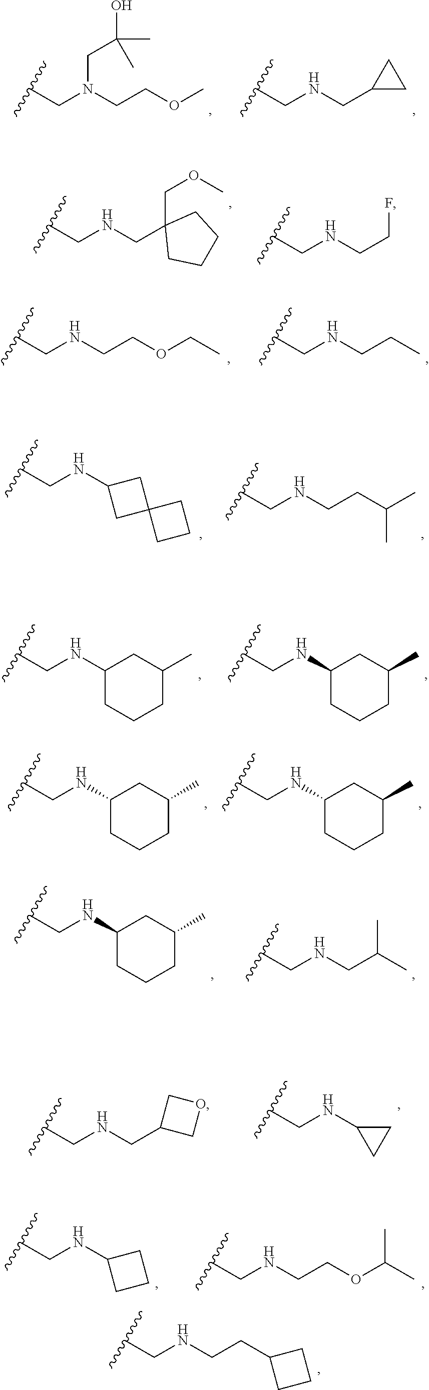

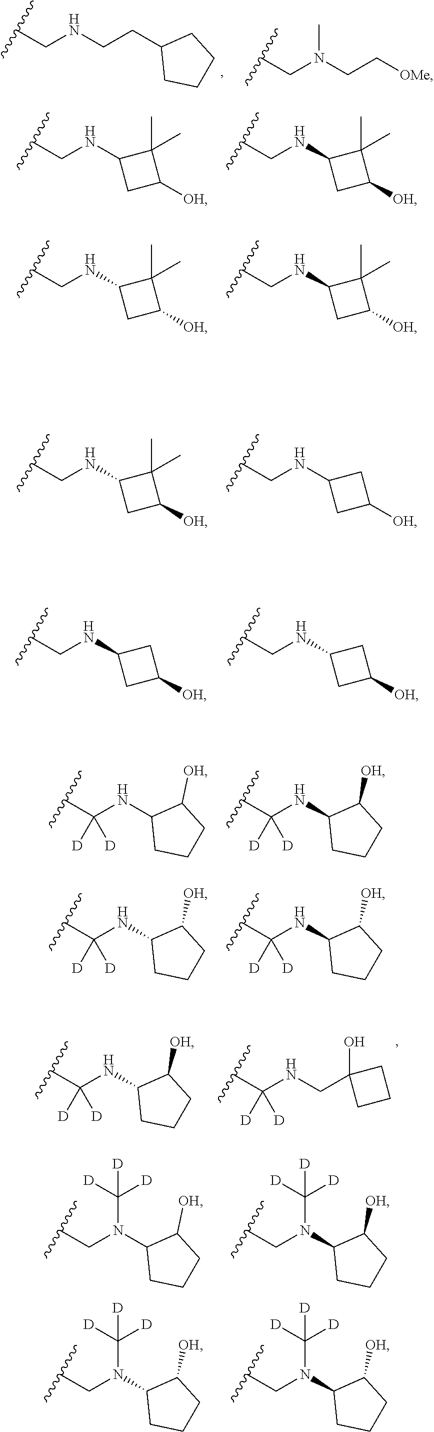

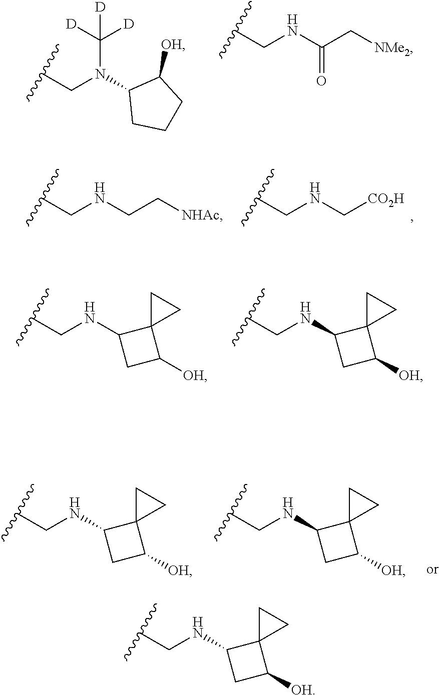

20. The compound of claim 1, wherein the compound is of formula II-a to II-j: 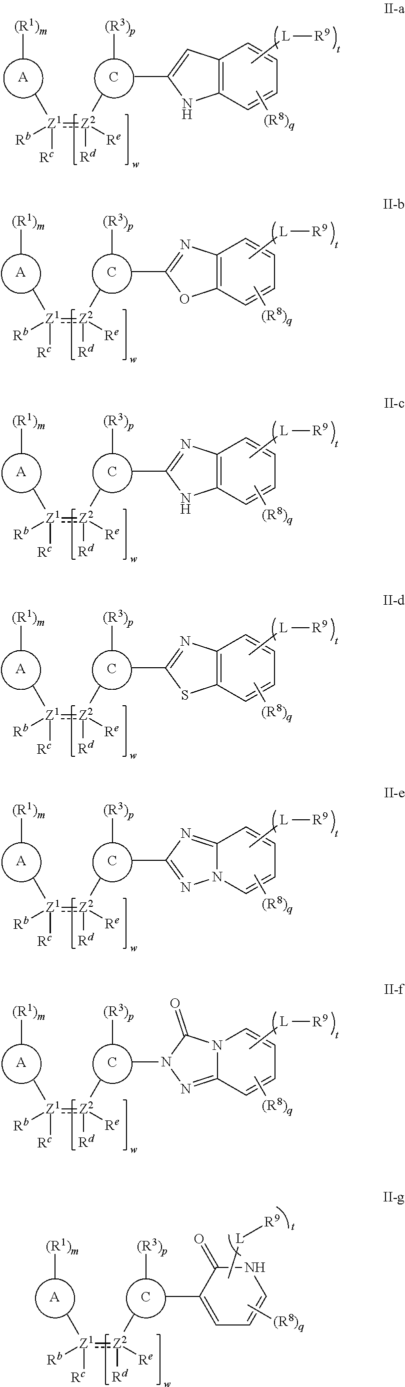 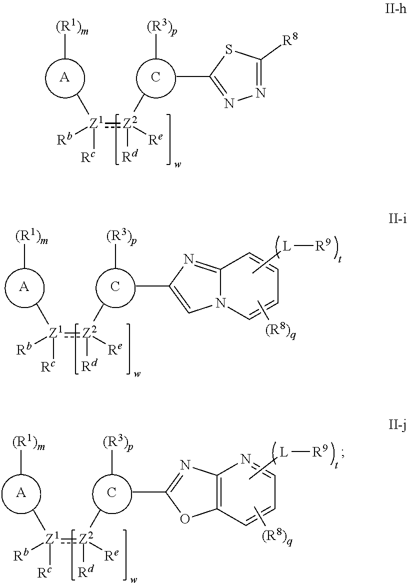
or a pharmaceutically acceptable salt thereof.

21. The compound of claim 1, wherein the compound is of formula II-b-1 to II-b-12: 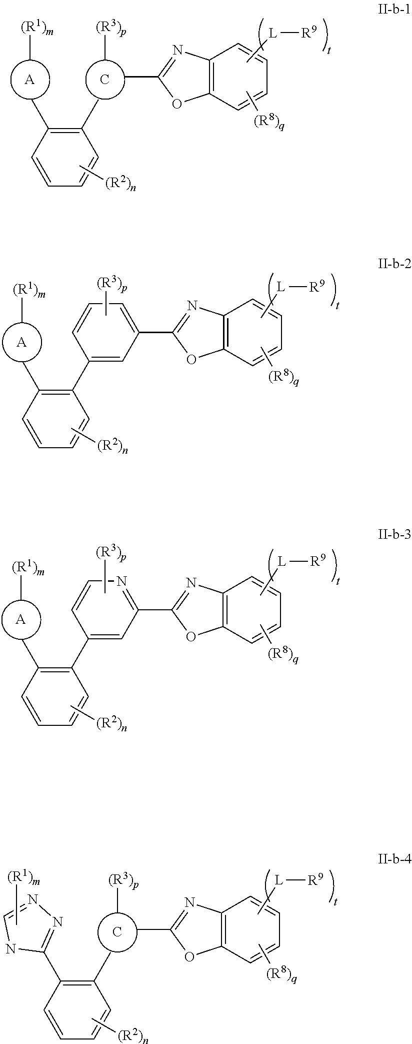 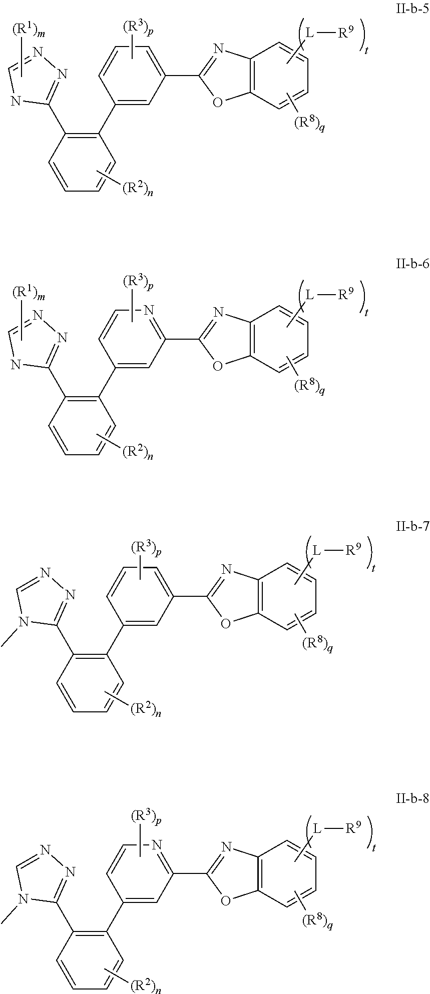 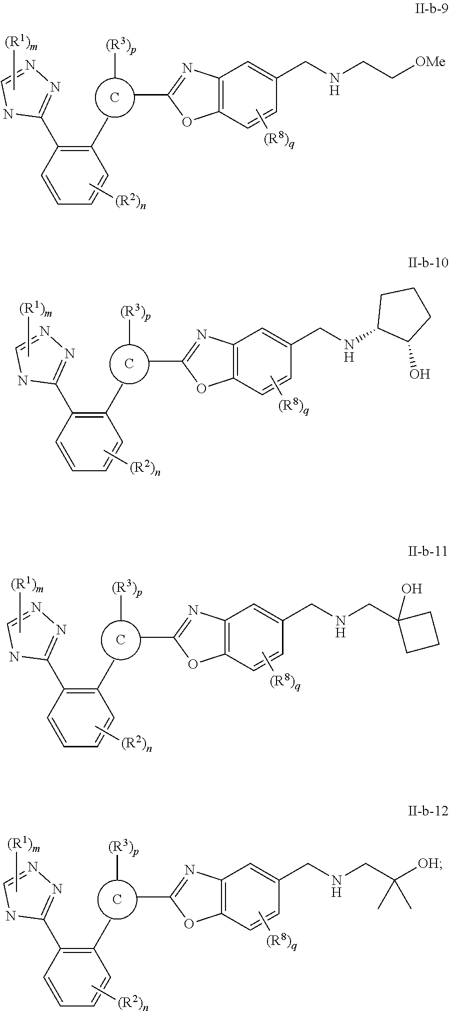
or a pharmaceutically acceptable salt thereof.

22. A compound selected from 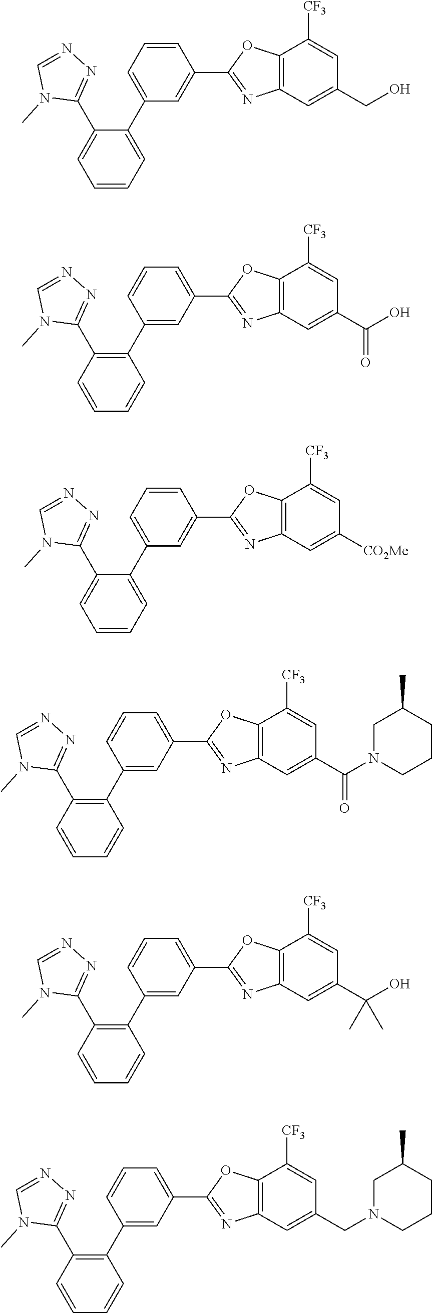 [SMILES: Cn1cnnc1-c1ccccc1-c1cccc(-c2nc3cc(CO)cc(C(F)(F)F)c3o2)c1 ; Cn1cnnc1-c1ccccc1-c1cccc(-c2nc3cc(C(=O)O)cc(C(F)(F)F)c3o2)c1 ; COC(=O)c1cc(C(F)(F)F)c2oc(-c3cccc(-c4ccccc4-c4nncn4C)c3)nc2c1 ; C[C@H]1CCCN(C(=O)c2cc(C(F)(F)F)c3oc(-c4cccc(-c5ccccc5-c5nncn5C)c4)nc3c2)C1 ; Cn1cnnc1-c1ccccc1-c1cccc(-c2nc3cc(C(C)(C)O)cc(C(F)(F)F)c3o2)c1 ; C[C@H]1CCCN(Cc2cc(C(F)(F)F)c3oc(-c4cccc(-c5ccccc5-c5nncn5C)c4)nc3c2)C1] 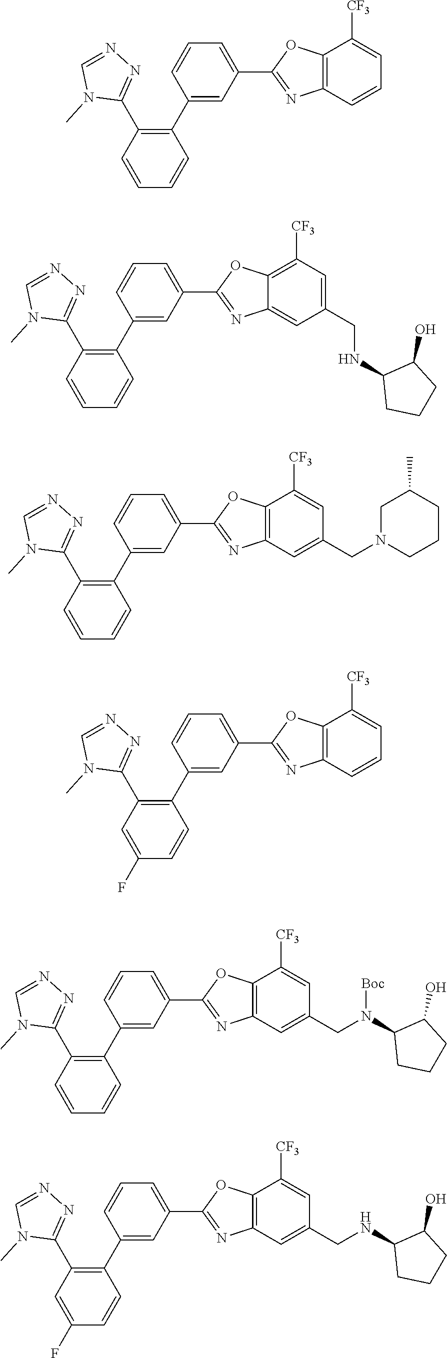 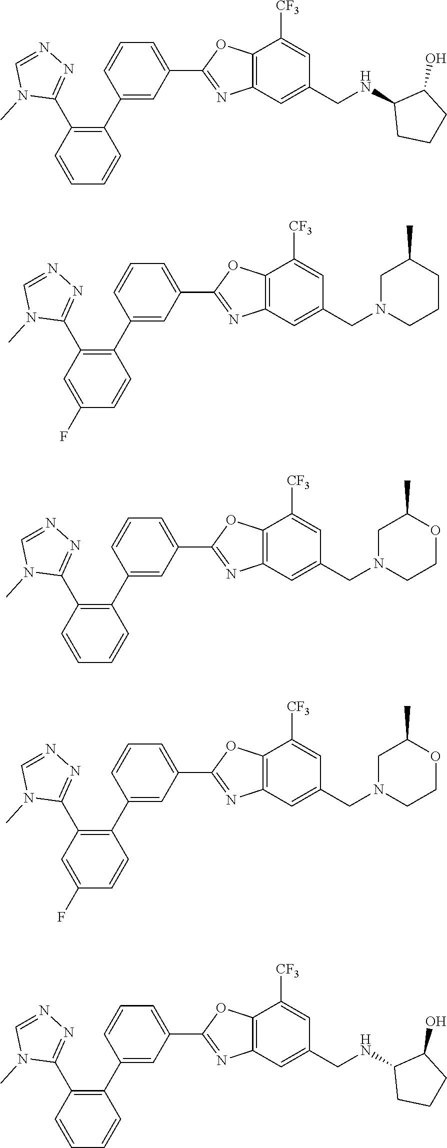 [SMILES: Cn1cnnc1-c1ccccc1-c1cccc(-c2nc3cc(CN[C@@H]4CCC[C@H]4O)cc(C(F)(F)F)c3o2)c1 ; C[C@H]1CCCN(Cc2cc(C(F)(F)F)c3oc(-c4cccc(-c5ccc(F)cc5-c5nncn5C)c4)nc3c2)C1 ; C[C@@H]1CN(Cc2cc(C(F)(F)F)c3oc(-c4cccc(-c5ccccc5-c5nncn5C)c4)nc3c2)CCO1 ; C[C@@H]1CN(Cc2cc(C(F)(F)F)c3oc(-c4cccc(-c5ccc(F)cc5-c5nncn5C)c4)nc3c2)CCO1 ; Cn1cnnc1-c1ccccc1-c1cccc(-c2nc3cc(CN[C@H]4CCC[C@@H]4O)cc(C(F)(F)F)c3o2)c1] 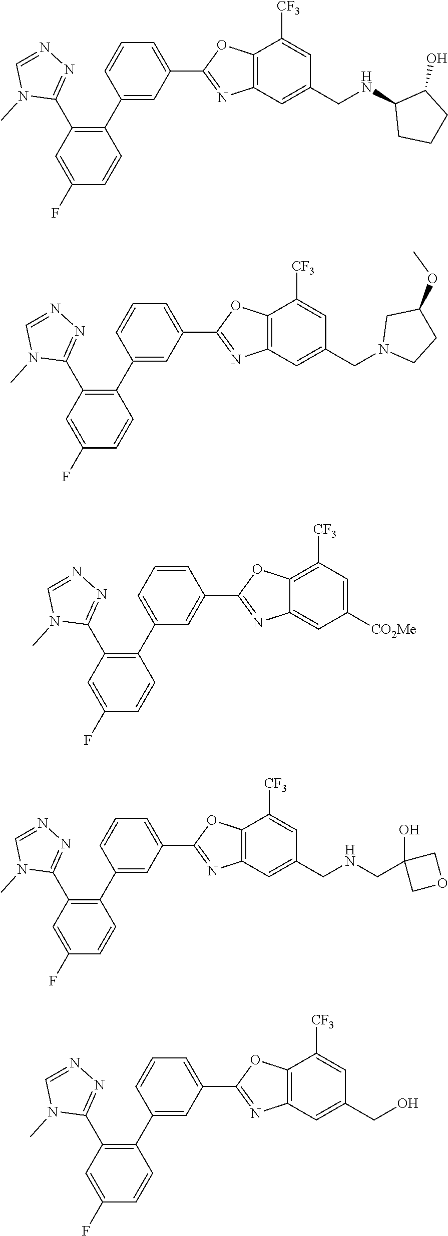 [SMILES: Cn1cnnc1-c1cc(F)ccc1-c1cccc(-c2nc3cc(CN[C@@H]4CCC[C@H]4O)cc(C(F)(F)F)c3o2)c1 ; CO[C@H]1CCN(Cc2cc(C(F)(F)F)c3oc(-c4cccc(-c5ccc(F)cc5-c5nncn5C)c4)nc3c2)C1 ; COC(=O)c1cc(C(F)(F)F)c2oc(-c3cccc(-c4ccc(F)cc4-c4nncn4C)c3)nc2c1 ; Cn1cnnc1-c1cc(F)ccc1-c1cccc(-c2nc3cc(CNCC4(O)COC4)cc(C(F)(F)F)c3o2)c1 ; Cn1cnnc1-c1cc(F)ccc1-c1cccc(-c2nc3cc(CO)cc(C(F)(F)F)c3o2)c1] 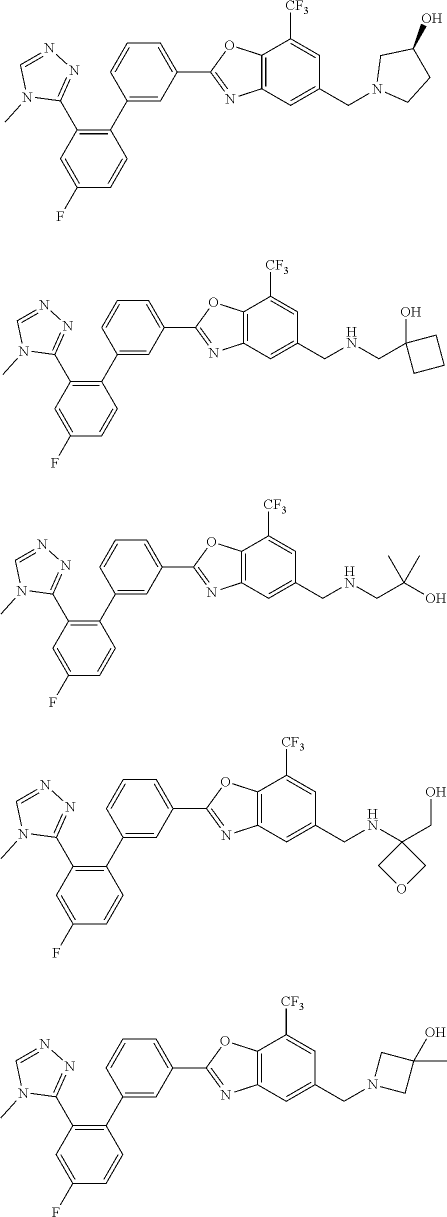 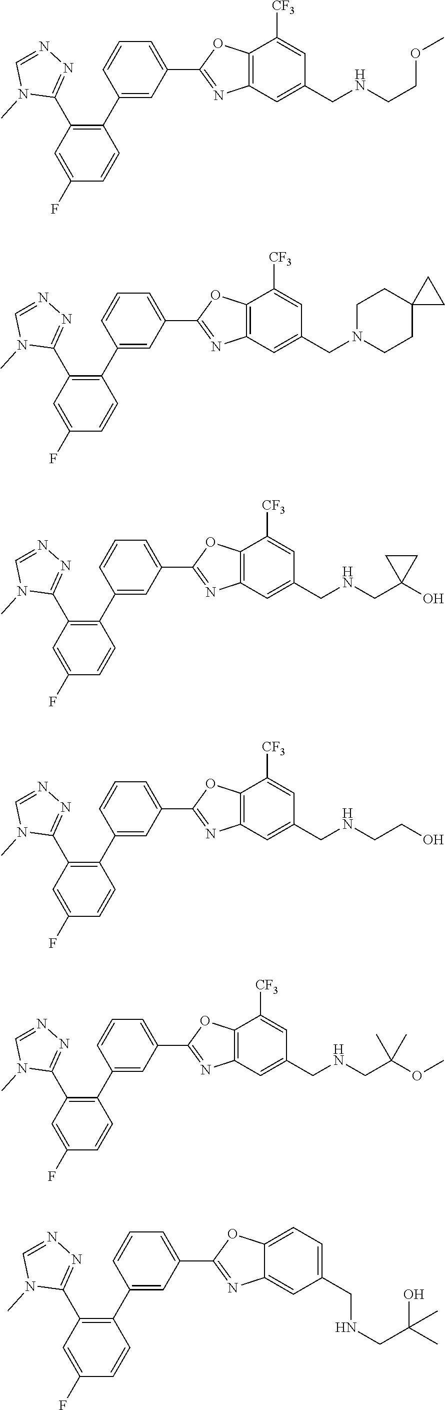 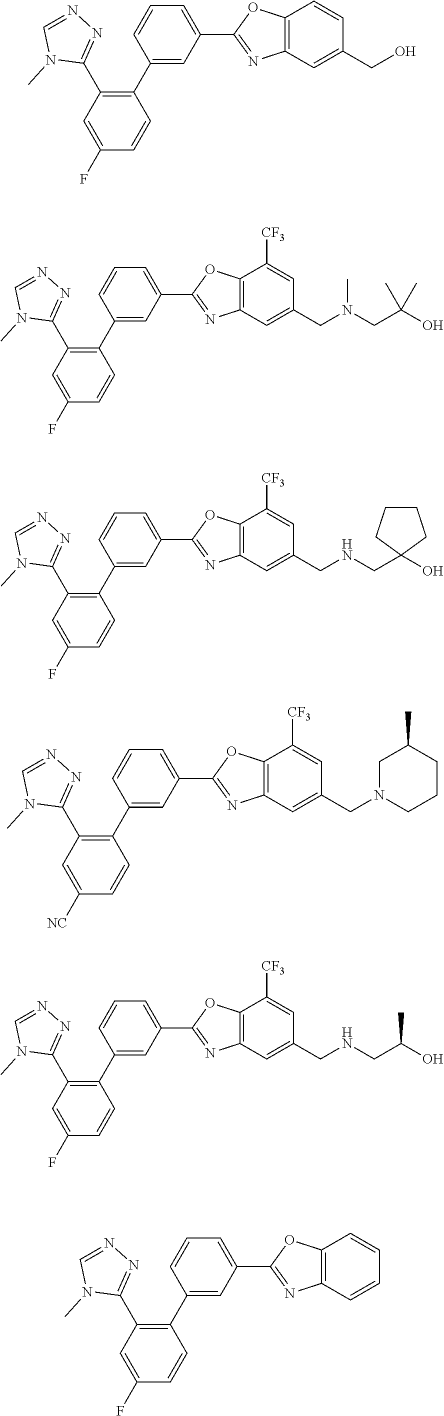 [SMILES: Cn1cnnc1-c1cc(F)ccc1-c1cccc(-c2nc3cc(CO)ccc3o2)c1 ; CN(Cc1cc(C(F)(F)F)c2oc(-c3cccc(-c4ccc(F)cc4-c4nncn4C)c3)nc2c1)CC(C)(C)O ; Cn1cnnc1-c1cc(F)ccc1-c1cccc(-c2nc3cc(CNCC4(O)CCCC4)cc(C(F)(F)F)c3o2)c1 ; C[C@H]1CCCN(Cc2cc(C(F)(F)F)c3oc(-c4cccc(-c5ccc(C#N)cc5-c5nncn5C)c4)nc3c2)C1 ; C[C@@H](O)CNCc1cc(C(F)(F)F)c2oc(-c3cccc(-c4ccc(F)cc4-c4nncn4C)c3)nc2c1 ; Cn1cnnc1-c1cc(F)ccc1-c1cccc(-c2nc3ccccc3o2)c1] 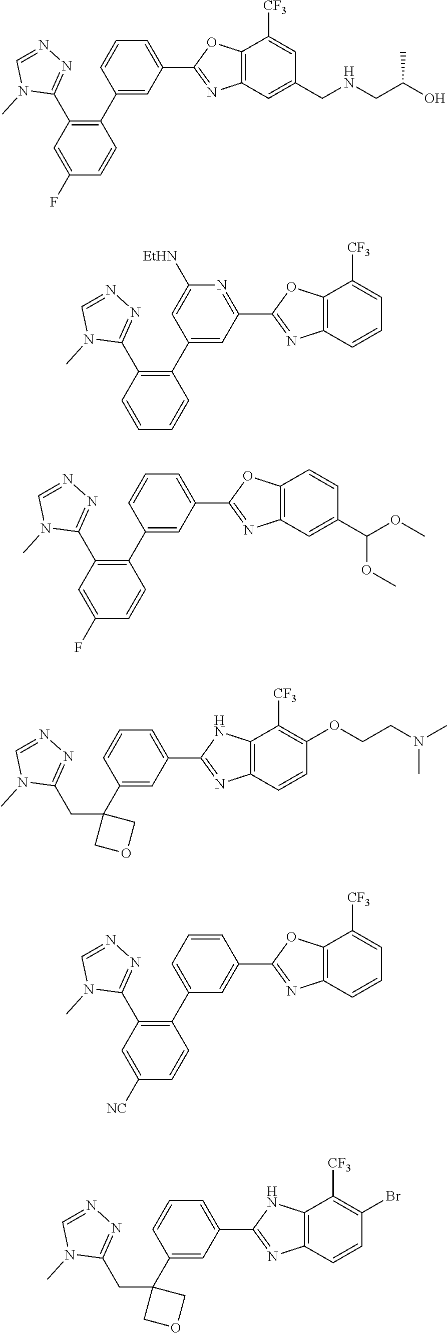 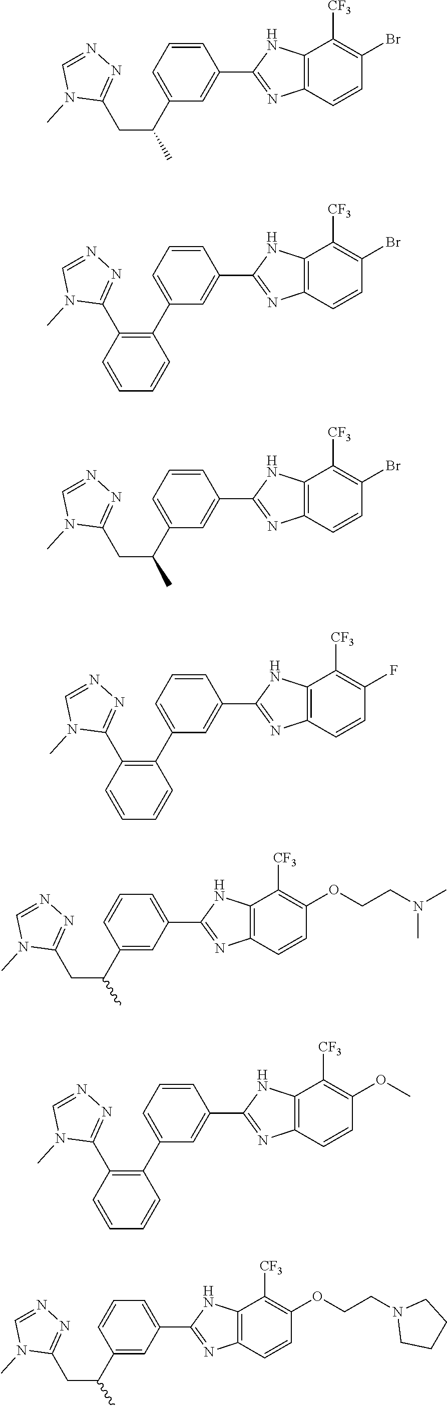 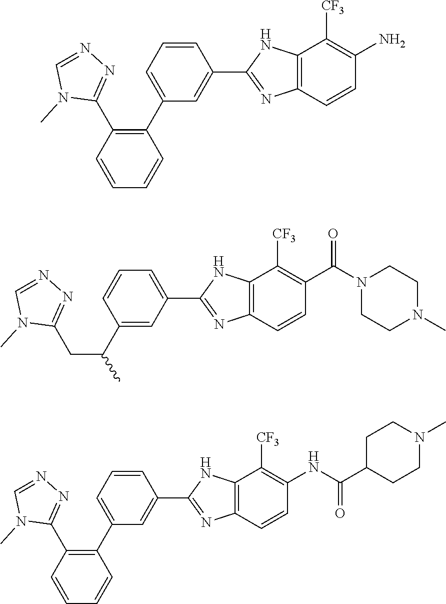 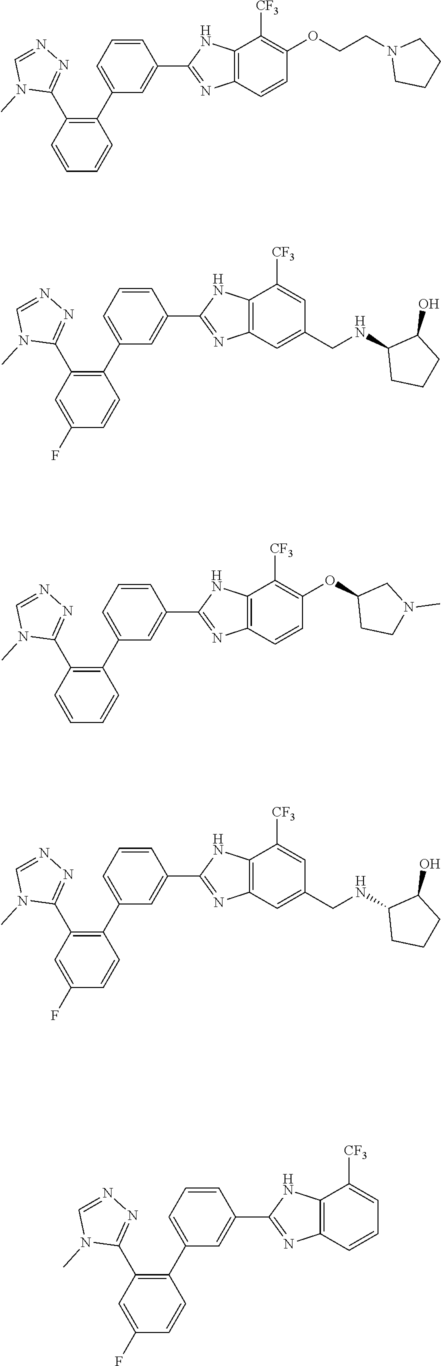 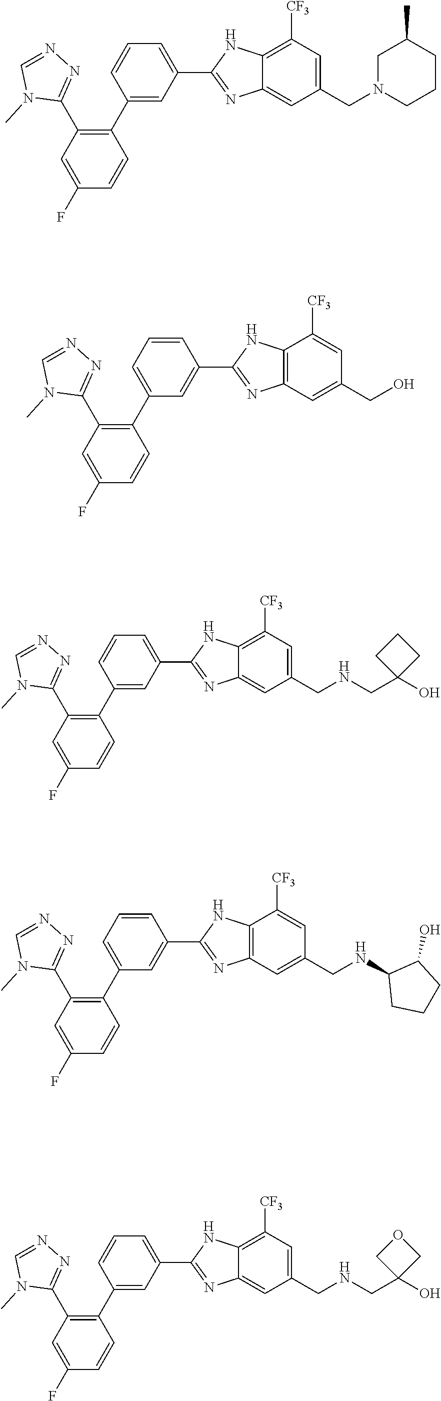 [SMILES: C[C@H]1CCCN(Cc2cc(C(F)(F)F)c3[nH]c(-c4cccc(-c5ccc(F)cc5-c5nncn5C)c4)nc3c2)C1 ; Cn1cnnc1-c1cc(F)ccc1-c1cccc(-c2nc3cc(CO)cc(C(F)(F)F)c3[nH]2)c1 ; Cn1cnnc1-c1cc(F)ccc1-c1cccc(-c2nc3cc(CNCC4(O)CCC4)cc(C(F)(F)F)c3[nH]2)c1 ; Cn1cnnc1-c1cc(F)ccc1-c1cccc(-c2nc3cc(CN[C@@H]4CCC[C@H]4O)cc(C(F)(F)F)c3[nH]2)c1 ; Cn1cnnc1-c1cc(F)ccc1-c1cccc(-c2nc3cc(CNCC4(O)COC4)cc(C(F)(F)F)c3[nH]2)c1] 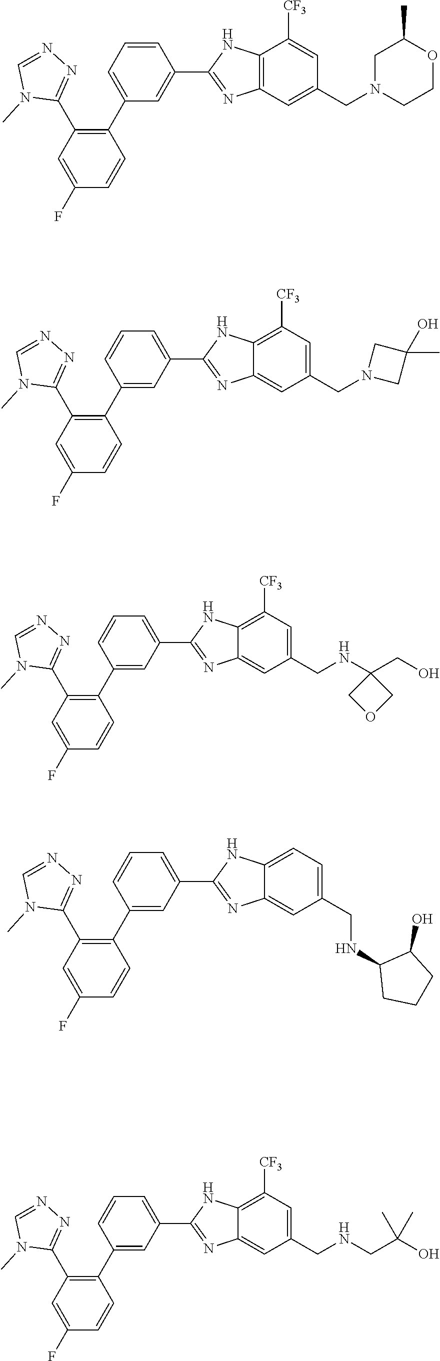 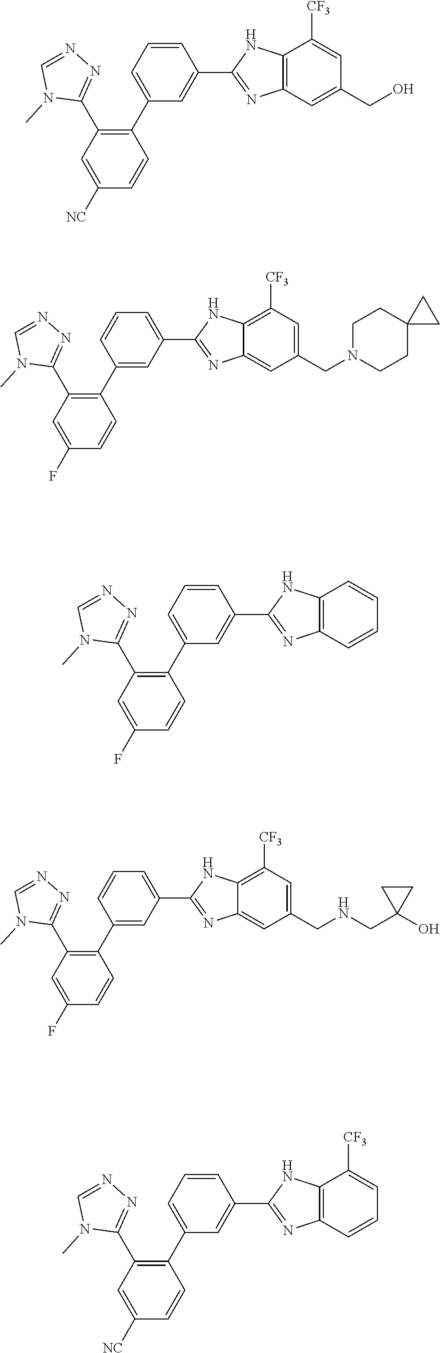 [SMILES: Cn1cnnc1-c1cc(C#N)ccc1-c1cccc(-c2nc3cc(CO)cc(C(F)(F)F)c3[nH]2)c1 ; Cn1cnnc1-c1cc(F)ccc1-c1cccc(-c2nc3cc(CN4CCC5(CC4)CC5)cc(C(F)(F)F)c3[nH]2)c1 ; Cn1cnnc1-c1cc(F)ccc1-c1cccc(-c2nc3ccccc3[nH]2)c1 ; Cn1cnnc1-c1cc(F)ccc1-c1cccc(-c2nc3cc(CNCC4(O)CC4)cc(C(F)(F)F)c3[nH]2)c1 ; Cn1cnnc1-c1cc(C#N)ccc1-c1cccc(-c2nc3cccc(C(F)(F)F)c3[nH]2)c1] 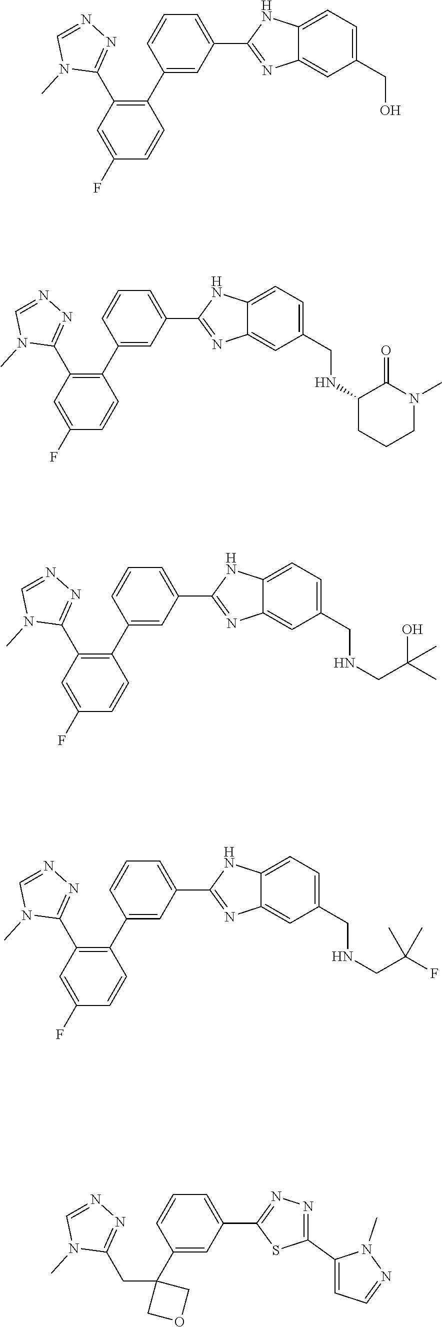 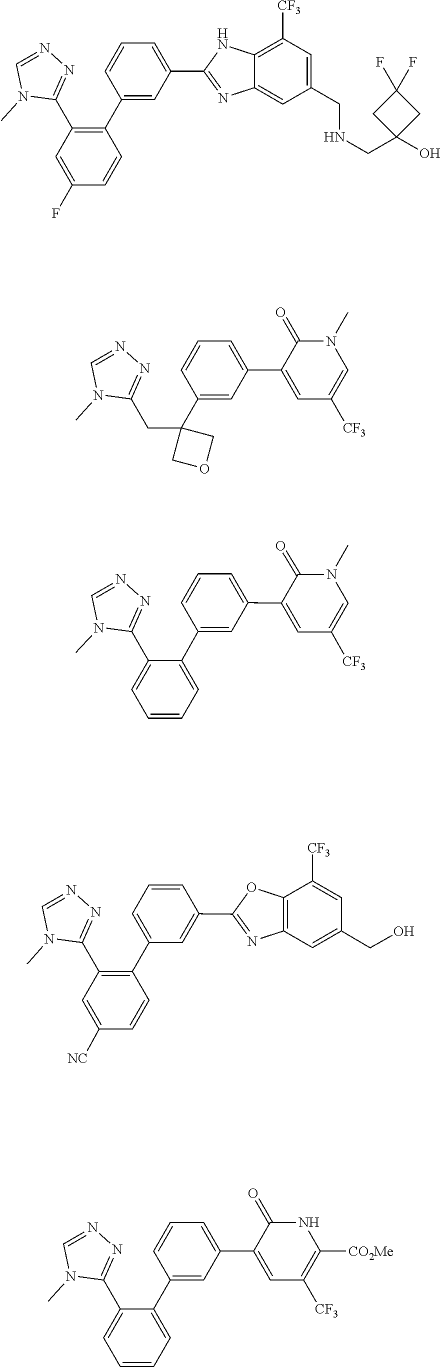 [SMILES: Cn1cnnc1-c1cc(F)ccc1-c1cccc(-c2nc3cc(CNCC4(O)CC(F)(F)C4)cc(C(F)(F)F)c3[nH]2)c1 ; Cn1cnnc1CC1(c2cccc(-c3cc(C(F)(F)F)cn(C)c3=O)c2)COC1 ; Cn1cnnc1-c1ccccc1-c1cccc(-c2cc(C(F)(F)F)cn(C)c2=O)c1 ; Cn1cnnc1-c1cc(C#N)ccc1-c1cccc(-c2nc3cc(CO)cc(C(F)(F)F)c3o2)c1 ; COC(=O)c1[nH]c(=O)c(-c2cccc(-c3ccccc3-c3nncn3C)c2)cc1C(F)(F)F] 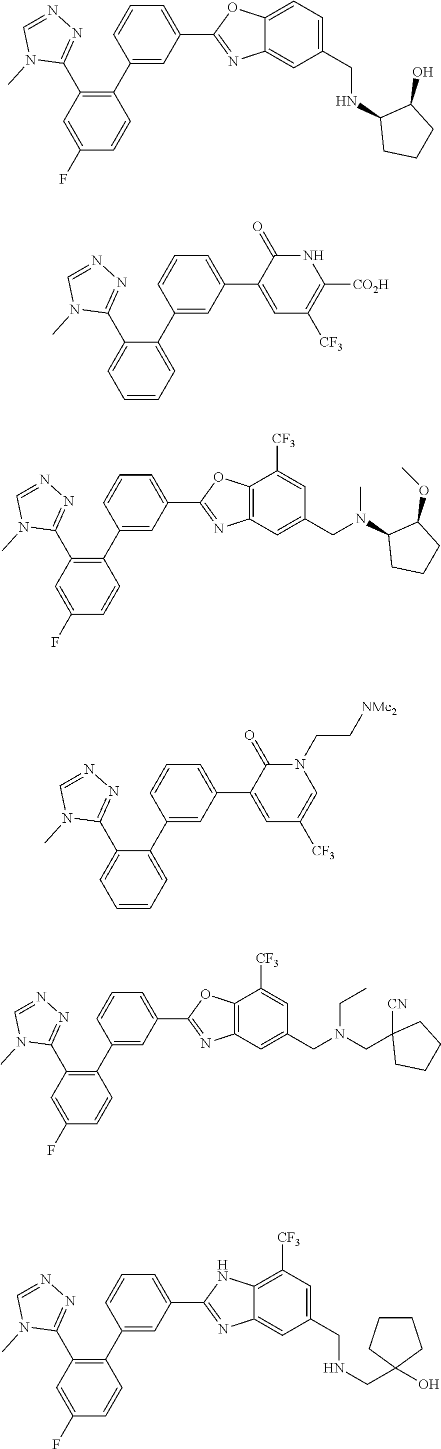 [SMILES: Cn1cnnc1-c1cc(F)ccc1-c1cccc(-c2nc3cc(CN[C@@H]4CCC[C@@H]4O)ccc3o2)c1 ; Cn1cnnc1-c1ccccc1-c1cccc(-c2cc(C(F)(F)F)c(C(=O)O)[nH]c2=O)c1 ; CO[C@H]1CCC[C@H]1N(C)Cc1cc(C(F)(F)F)c2oc(-c3cccc(-c4ccc(F)cc4-c4nncn4C)c3)nc2c1 ; CN(C)CCn1cc(C(F)(F)F)cc(-c2cccc(-c3ccccc3-c3nncn3C)c2)c1=O ; CCN(Cc1cc(C(F)(F)F)c2oc(-c3cccc(-c4ccc(F)cc4-c4nncn4C)c3)nc2c1)CC1(C#N)CCCC1 ; Cn1cnnc1-c1cc(F)ccc1-c1cccc(-c2nc3cc(CNCC4(O)CCCC4)cc(C(F)(F)F)c3[nH]2)c1] 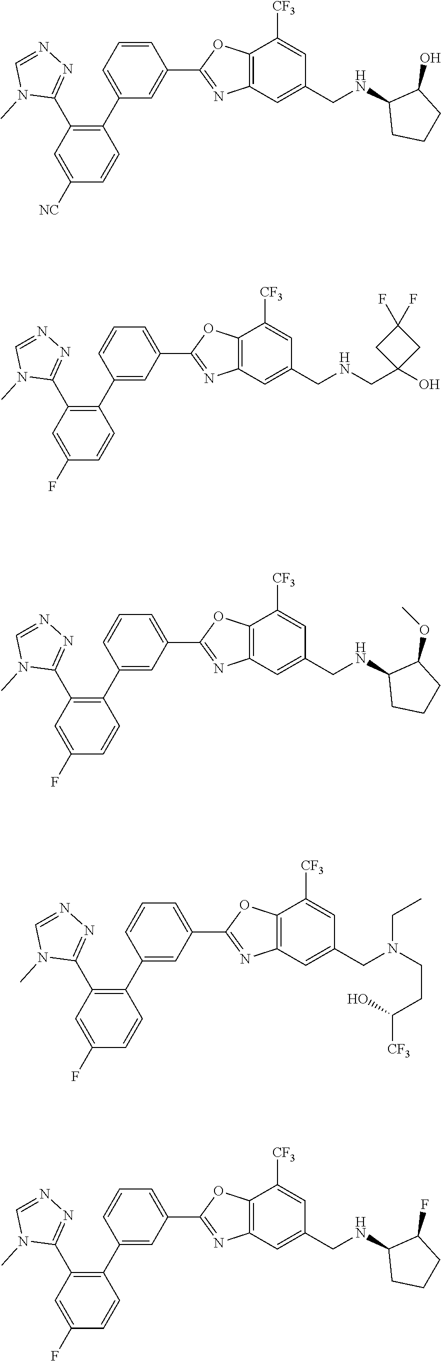 [SMILES: Cn1cnnc1-c1cc(C#N)ccc1-c1cccc(-c2nc3cc(CN[C@@H]4CCC[C@@H]4O)cc(C(F)(F)F)c3o2)c1 ; Cn1cnnc1-c1cc(F)ccc1-c1cccc(-c2nc3cc(CNCC4(O)CC(F)(F)C4)cc(C(F)(F)F)c3o2)c1 ; CO[C@H]1CCC[C@H]1NCc1cc(C(F)(F)F)c2oc(-c3cccc(-c4ccc(F)cc4-c4nncn4C)c3)nc2c1 ; CCN(CC[C@@H](O)C(F)(F)F)Cc1cc(C(F)(F)F)c2oc(-c3cccc(-c4ccc(F)cc4-c4nncn4C)c3)nc2c1 ; Cn1cnnc1-c1cc(F)ccc1-c1cccc(-c2nc3cc(CN[C@@H]4CCC[C@@H]4F)cc(C(F)(F)F)c3o2)c1] 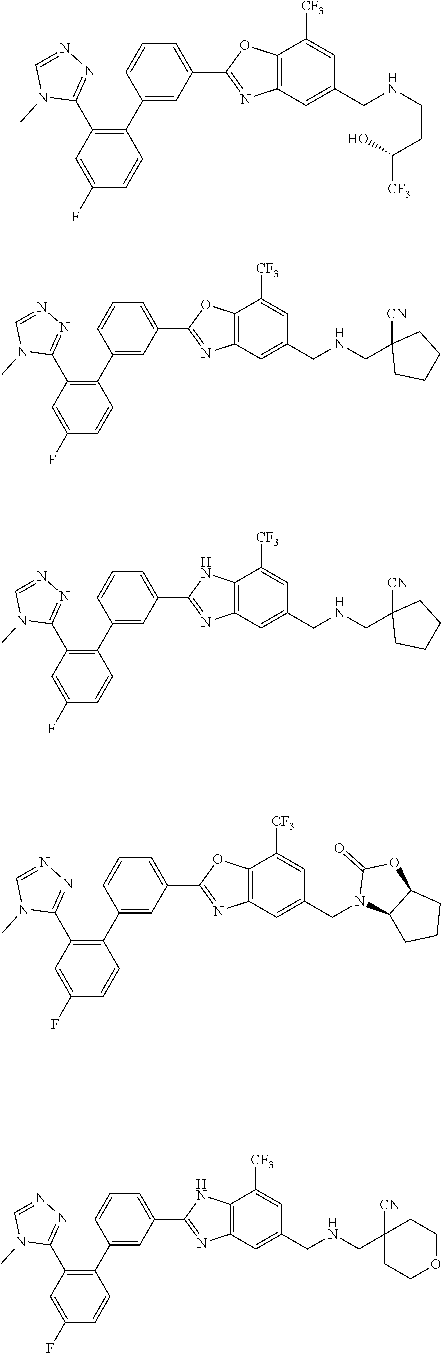 [SMILES: Cn1cnnc1-c1cc(F)ccc1-c1cccc(-c2nc3cc(CNCC[C@@H](O)C(F)(F)F)cc(C(F)(F)F)c3o2)c1 ; Cn1cnnc1-c1cc(F)ccc1-c1cccc(-c2nc3cc(CNCC4(C#N)CCCC4)cc(C(F)(F)F)c3o2)c1 ; Cn1cnnc1-c1cc(F)ccc1-c1cccc(-c2nc3cc(CNCC4(C#N)CCCC4)cc(C(F)(F)F)c3[nH]2)c1 ; Cn1cnnc1-c1cc(F)ccc1-c1cccc(-c2nc3cc(CN4C(=O)O[C@H]5CCC[C@H]54)cc(C(F)(F)F)c3o2)c1 ; Cn1cnnc1-c1cc(F)ccc1-c1cccc(-c2nc3cc(CNCC4(C#N)CCOCC4)cc(C(F)(F)F)c3[nH]2)c1] 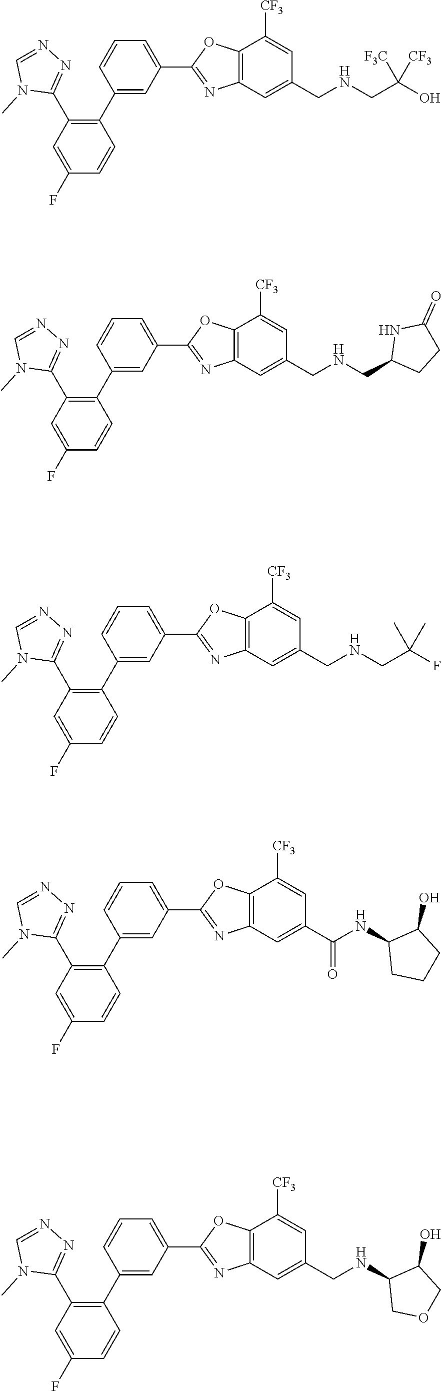 [SMILES: Cn1cnnc1-c1cc(F)ccc1-c1cccc(-c2nc3cc(CNCC(O)(C(F)(F)F)C(F)(F)F)cc(C(F)(F)F)c3o2)c1 ; Cn1cnnc1-c1cc(F)ccc1-c1cccc(-c2nc3cc(CNC[C@@H]4CCC(=O)N4)cc(C(F)(F)F)c3o2)c1 ; Cn1cnnc1-c1cc(F)ccc1-c1cccc(-c2nc3cc(CNCC(C)(C)F)cc(C(F)(F)F)c3o2)c1 ; Cn1cnnc1-c1cc(F)ccc1-c1cccc(-c2nc3cc(C(=O)N[C@@H]4CCC[C@@H]4O)cc(C(F)(F)F)c3o2)c1 ; Cn1cnnc1-c1cc(F)ccc1-c1cccc(-c2nc3cc(CN[C@@H]4COC[C@@H]4O)cc(C(F)(F)F)c3o2)c1] 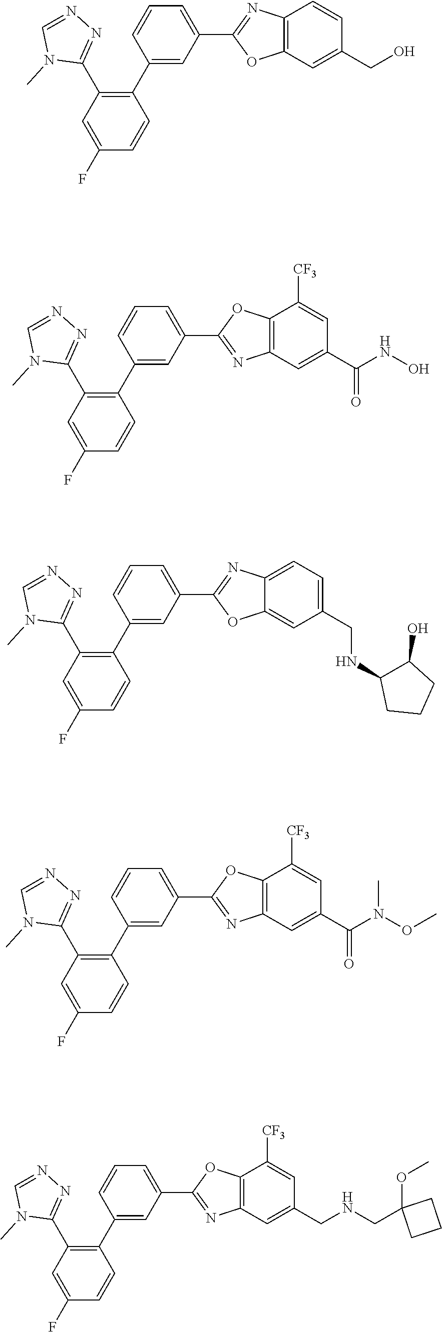 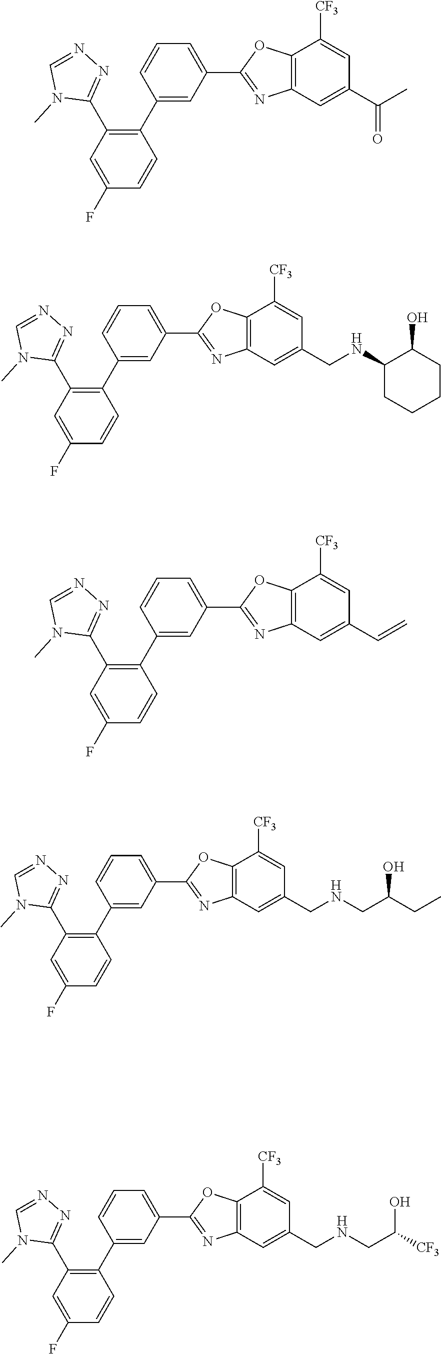 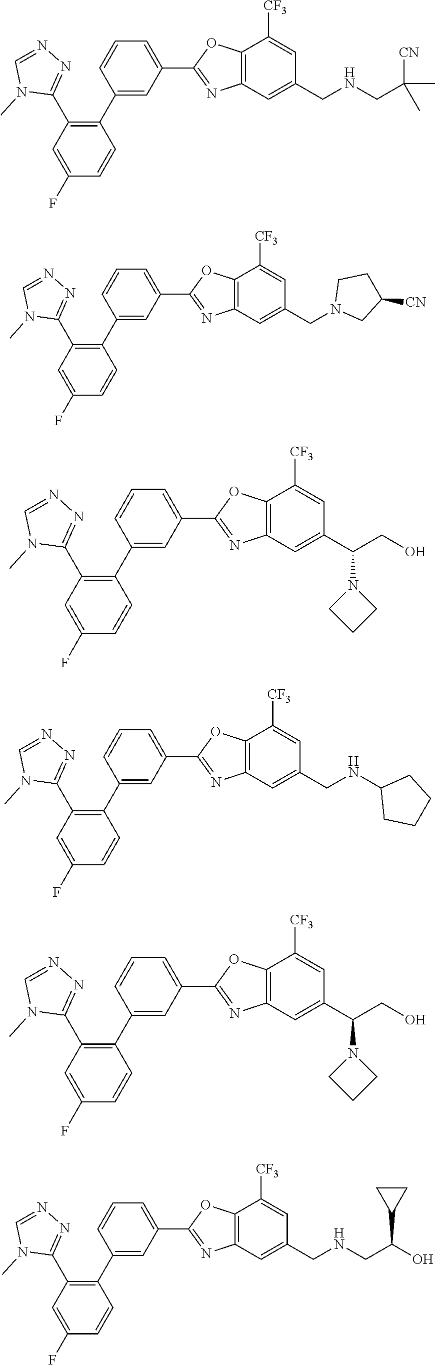 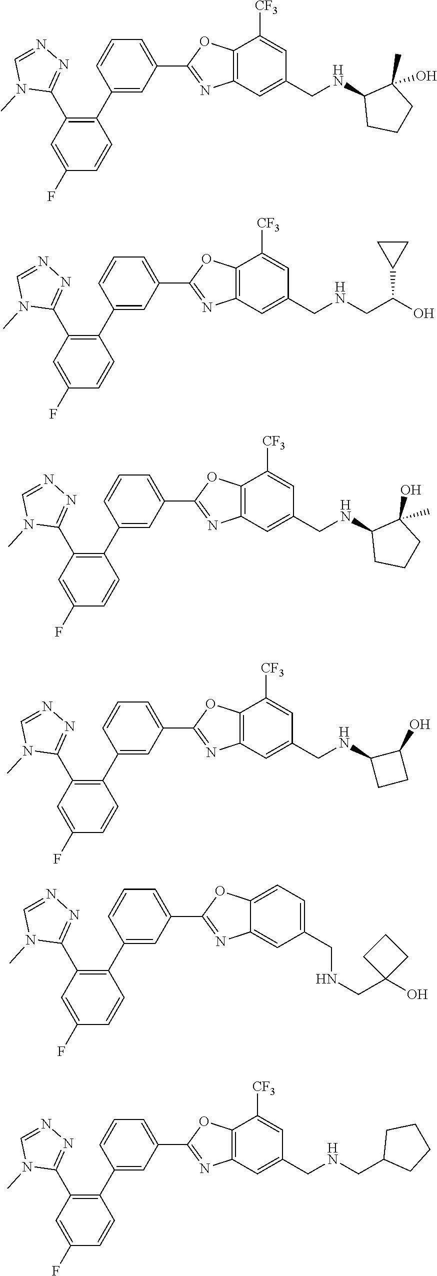 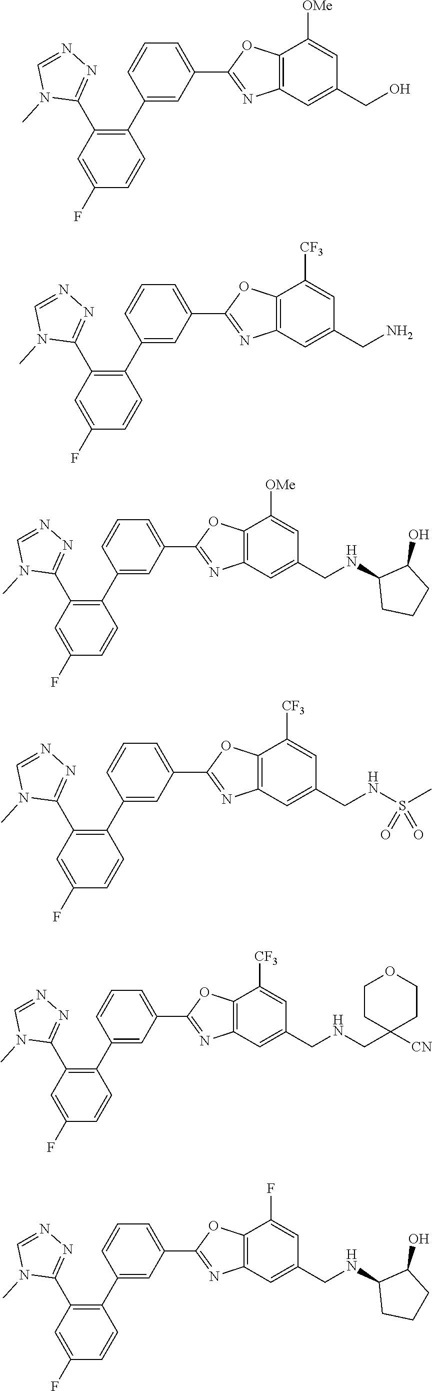 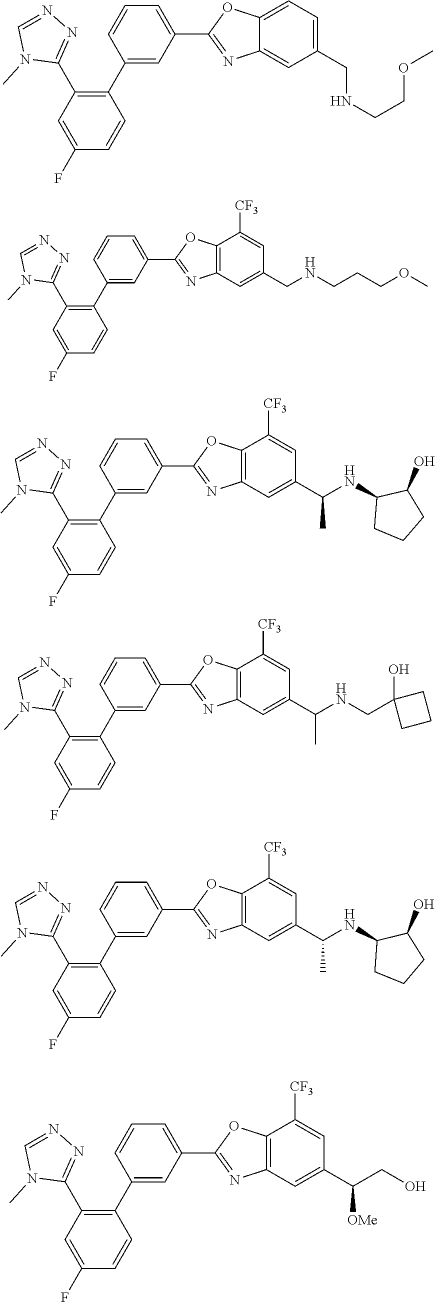 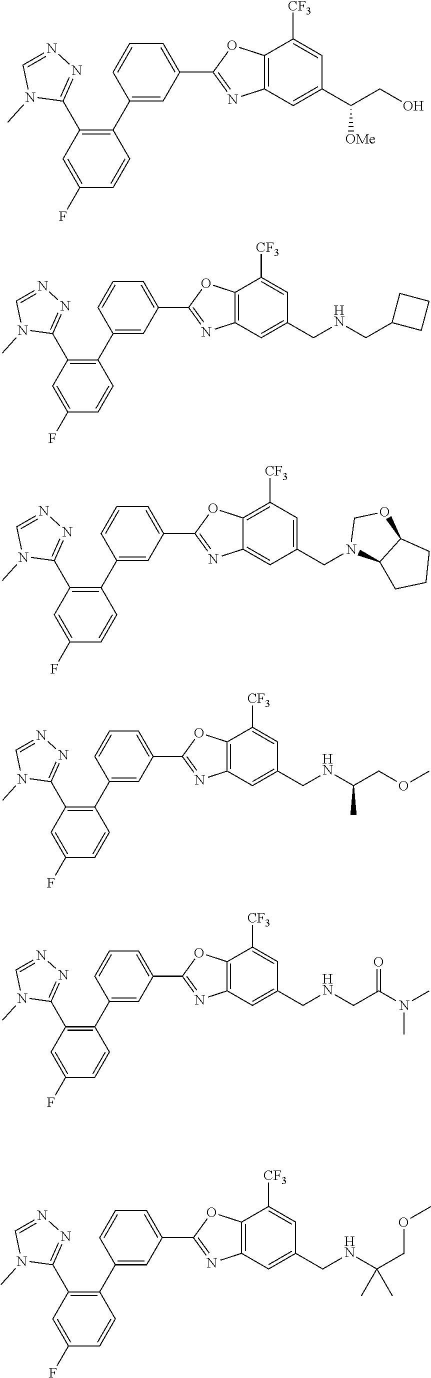 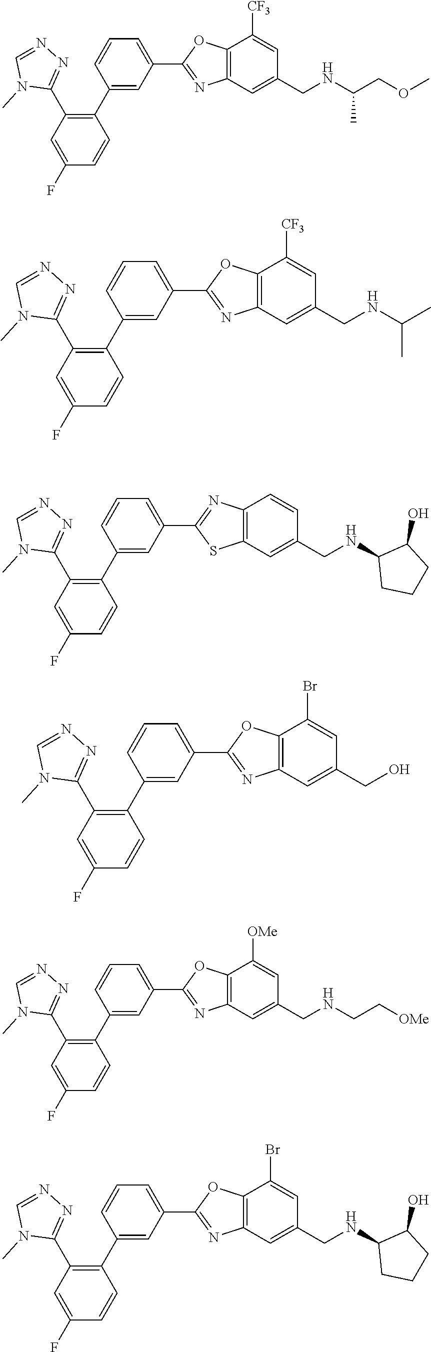 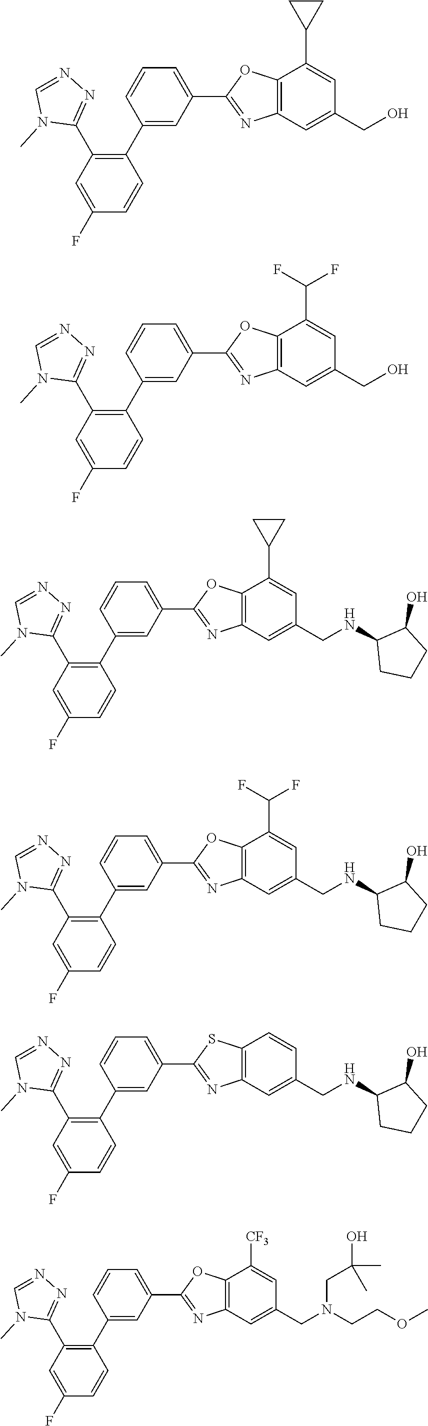 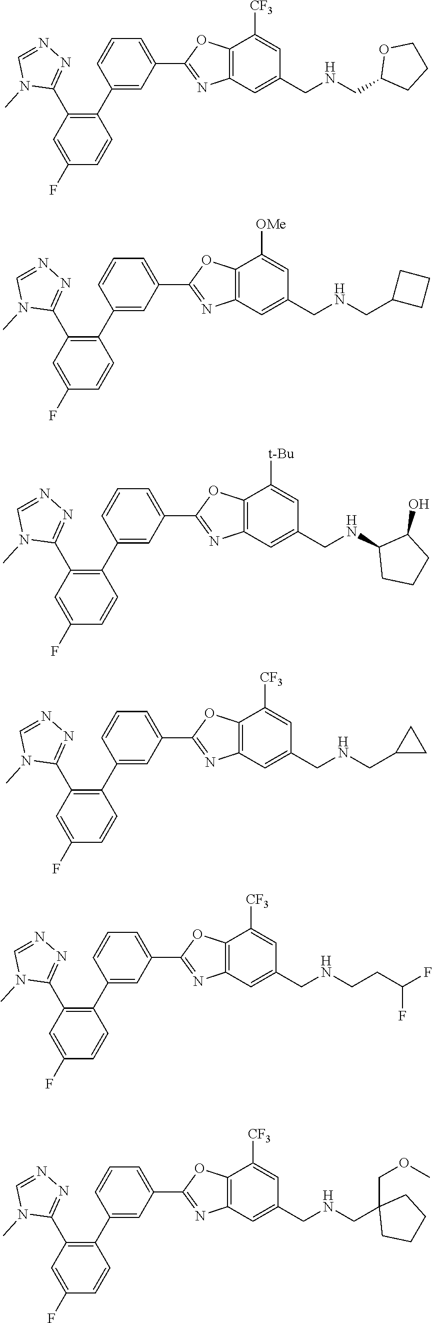 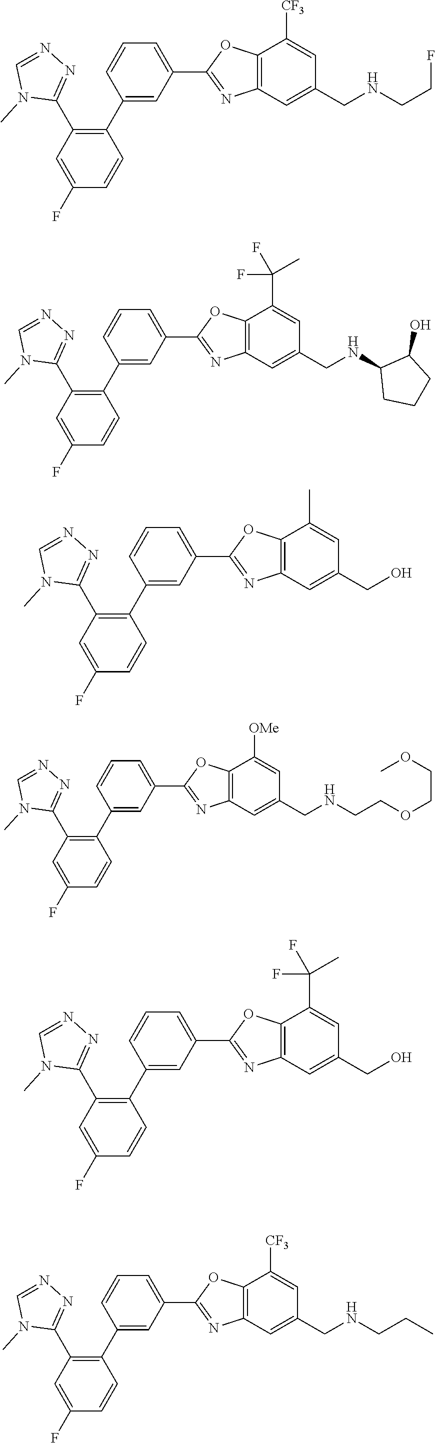 [SMILES: Cn1cnnc1-c1cc(F)ccc1-c1cccc(-c2nc3cc(CNCCF)cc(C(F)(F)F)c3o2)c1 ; Cn1cnnc1-c1cc(F)ccc1-c1cccc(-c2nc3cc(CN[C@@H]4CCC[C@@H]4O)cc(C(C)(F)F)c3o2)c1 ; Cc1cc(CO)cc2nc(-c3cccc(-c4ccc(F)cc4-c4nncn4C)c3)oc12 ; COCCOCCNCc1cc(OC)c2oc(-c3cccc(-c4ccc(F)cc4-c4nncn4C)c3)nc2c1 ; Cn1cnnc1-c1cc(F)ccc1-c1cccc(-c2nc3cc(CO)cc(C(C)(F)F)c3o2)c1 ; CCCNCc1cc(C(F)(F)F)c2oc(-c3cccc(-c4ccc(F)cc4-c4nncn4C)c3)nc2c1] 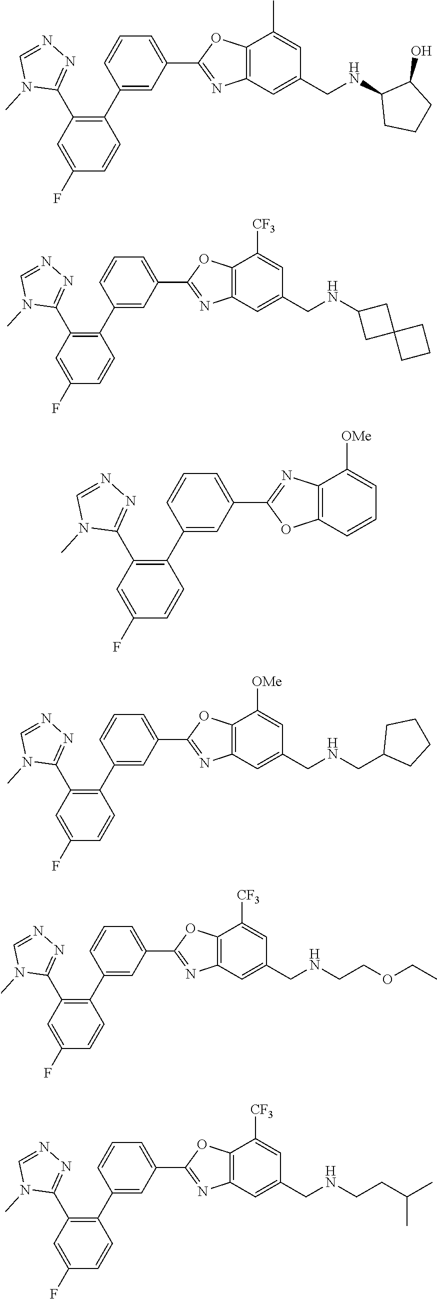 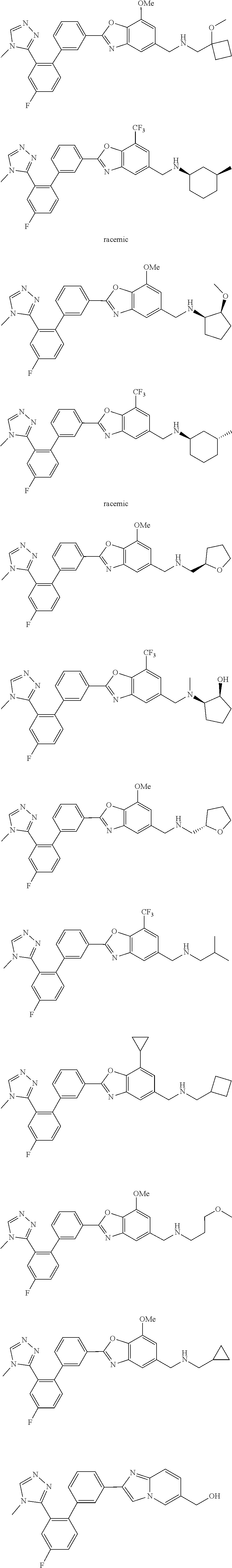 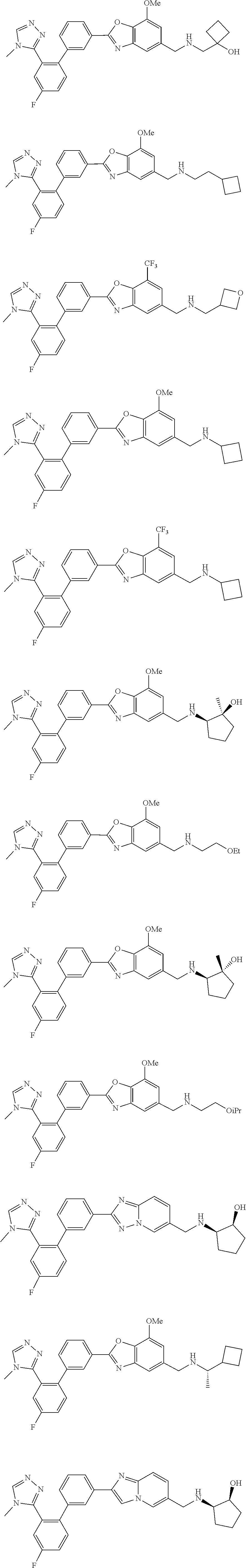 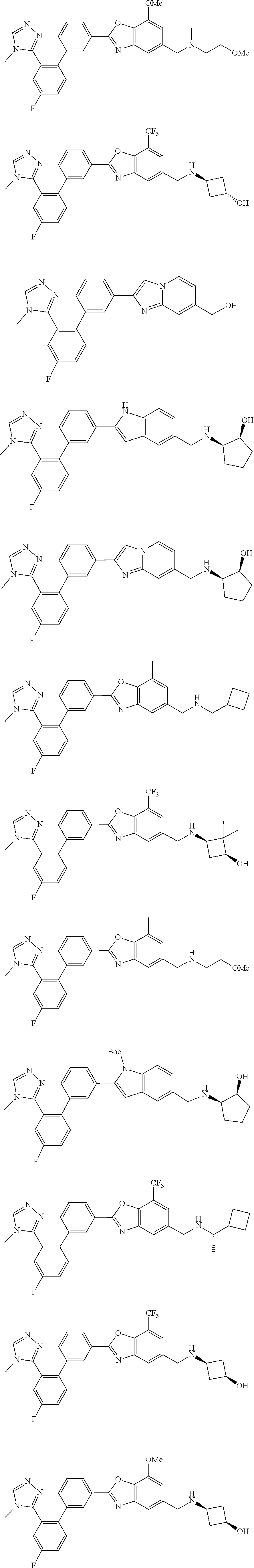 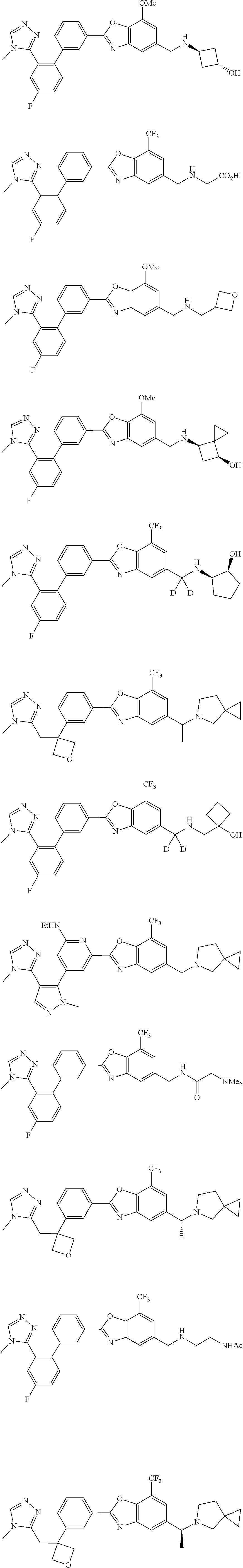 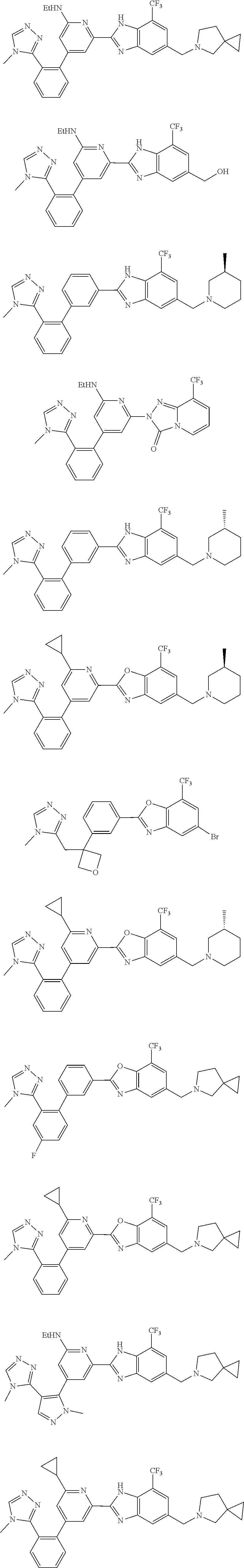 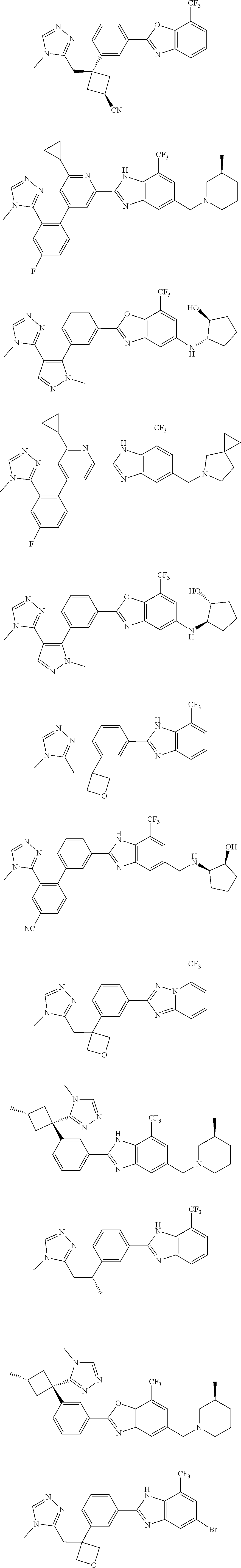 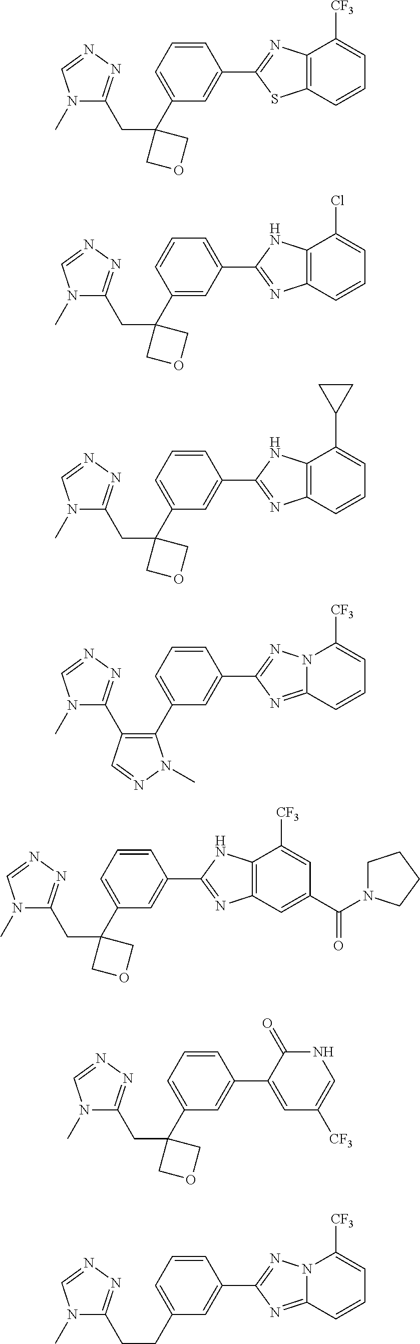 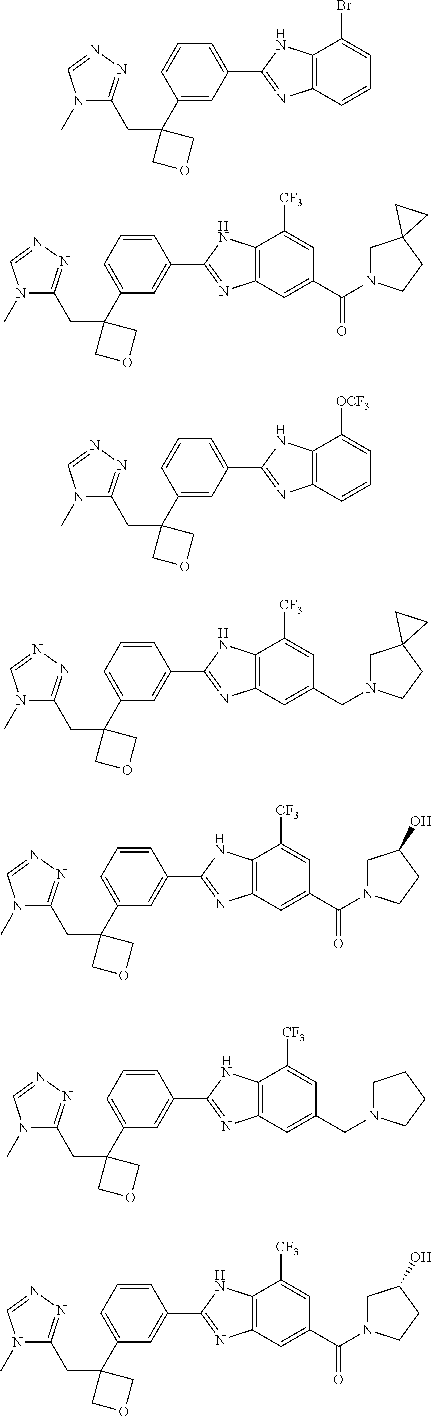 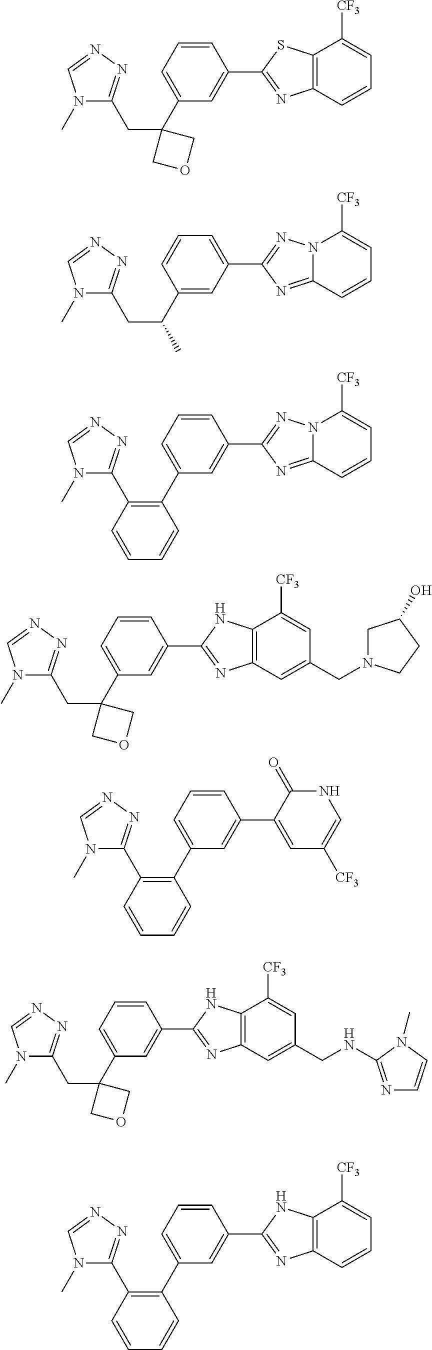 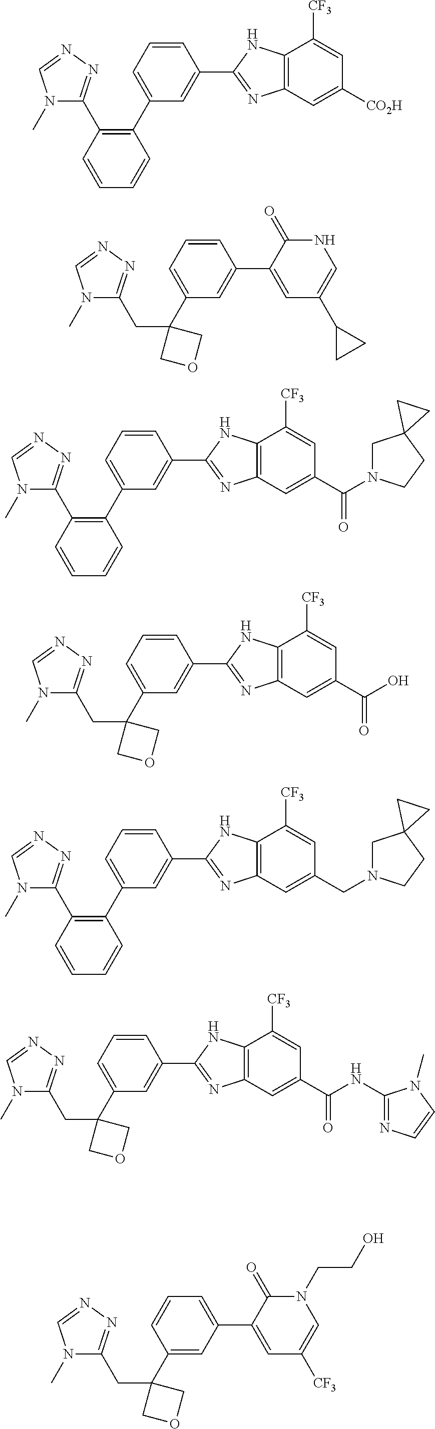 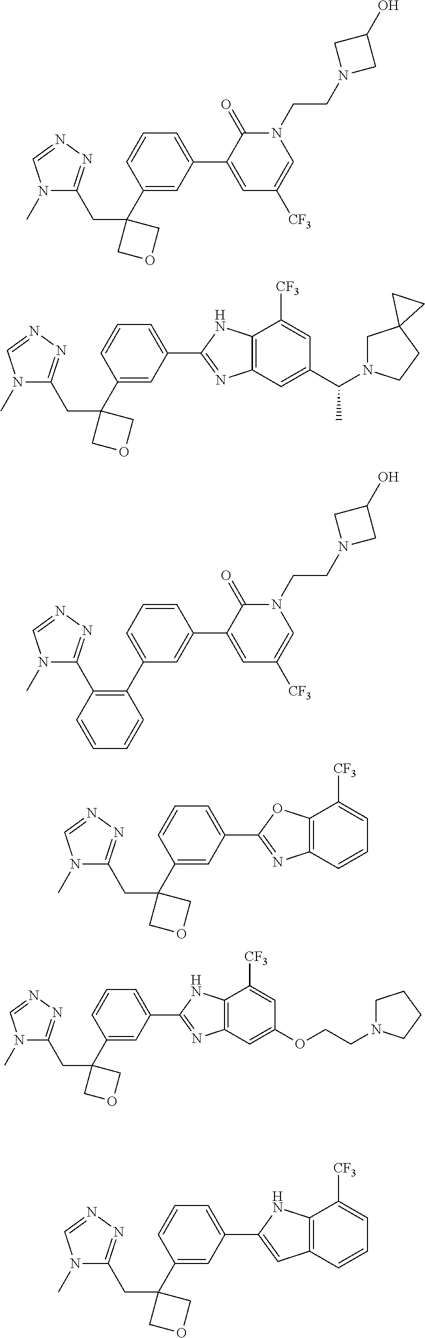 [SMILES: Cn1cnnc1CC1(c2cccc(-c3cc(C(F)(F)F)cn(CCN4CC(O)C4)c3=O)c2)COC1 ; C[C@H](c1cc(C(F)(F)F)c2[nH]c(-c3cccc(C4(Cc5nncn5C)COC4)c3)nc2c1)N1CCC2(CC2)C1 ; Cn1cnnc1-c1ccccc1-c1cccc(-c2cc(C(F)(F)F)cn(CCN3CC(O)C3)c2=O)c1 ; Cn1cnnc1CC1(c2cccc(-c3nc4cccc(C(F)(F)F)c4o3)c2)COC1 ; Cn1cnnc1CC1(c2cccc(-c3nc4cc(OCCN5CCCC5)cc(C(F)(F)F)c4[nH]3)c2)COC1 ; Cn1cnnc1CC1(c2cccc(-c3cc4cccc(C(F)(F)F)c4[nH]3)c2)COC1] 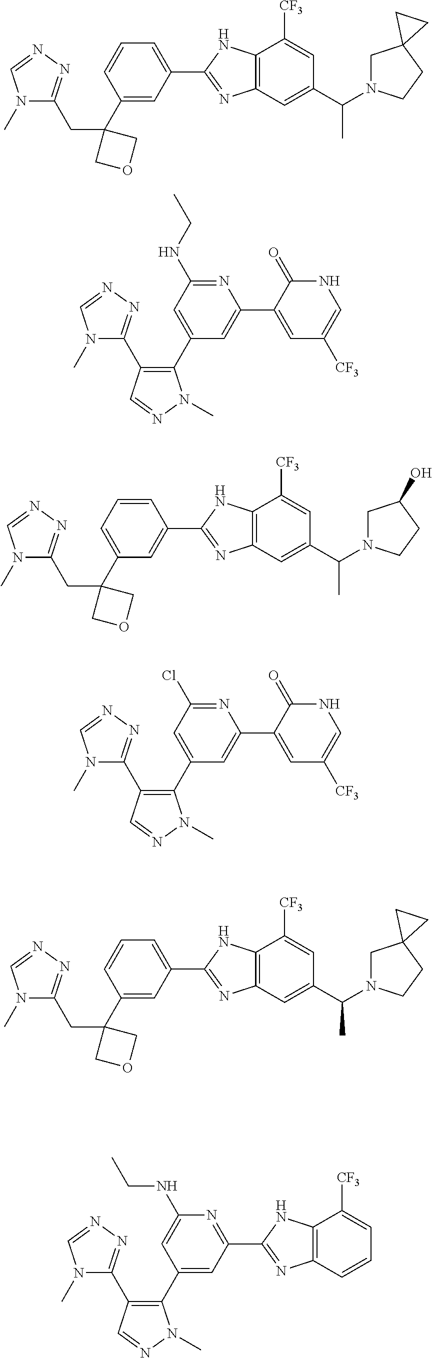 [SMILES: CC(c1cc(C(F)(F)F)c2[nH]c(-c3cccc(C4(Cc5nncn5C)COC4)c3)nc2c1)N1CCC2(CC2)C1 ; CCNc1cc(-c2c(-c3nncn3C)cnn2C)cc(-c2cc(C(F)(F)F)c[nH]c2=O)n1 ; CC(c1cc(C(F)(F)F)c2[nH]c(-c3cccc(C4(Cc5nncn5C)COC4)c3)nc2c1)N1CC[C@H](O)C1 ; Cn1cnnc1-c1cnn(C)c1-c1cc(Cl)nc(-c2cc(C(F)(F)F)c[nH]c2=O)c1 ; C[C@@H](c1cc(C(F)(F)F)c2[nH]c(-c3cccc(C4(Cc5nncn5C)COC4)c3)nc2c1)N1CCC2(CC2)C1 ; CCNc1cc(-c2c(-c3nncn3C)cnn2C)cc(-c2nc3cccc(C(F)(F)F)c3[nH]2)n1] 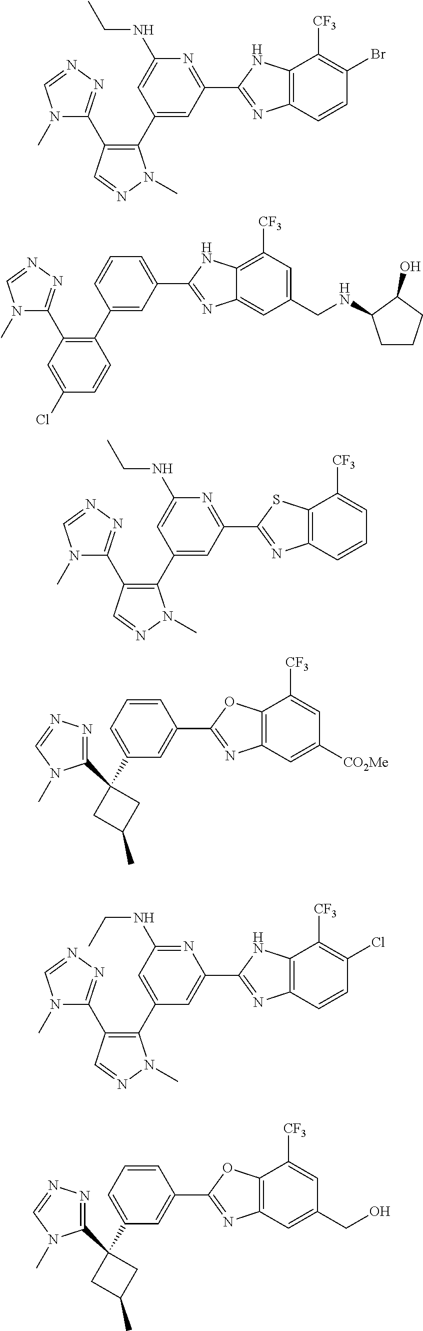 [SMILES: CCNc1cc(-c2c(-c3nncn3C)cnn2C)cc(-c2nc3ccc(Br)c(C(F)(F)F)c3[nH]2)n1 ; Cn1cnnc1-c1cc(Cl)ccc1-c1cccc(-c2nc3cc(CN[C@@H]4CCC[C@@H]4O)cc(C(F)(F)F)c3[nH]2)c1 ; CCNc1cc(-c2c(-c3nncn3C)cnn2C)cc(-c2nc3cccc(C(F)(F)F)c3s2)n1 ; COC(=O)c1cc(C(F)(F)F)c2oc(-c3cccc([C@]4(c5nncn5C)C[C@@H](C)C4)c3)nc2c1 ; CCNc1cc(-c2c(-c3nncn3C)cnn2C)cc(-c2nc3ccc(Cl)c(C(F)(F)F)c3[nH]2)n1 ; Cn1cnnc1[C@]1(c2cccc(-c3nc4cc(CO)cc(C(F)(F)F)c4o3)c2)C[C@H](C)C1] 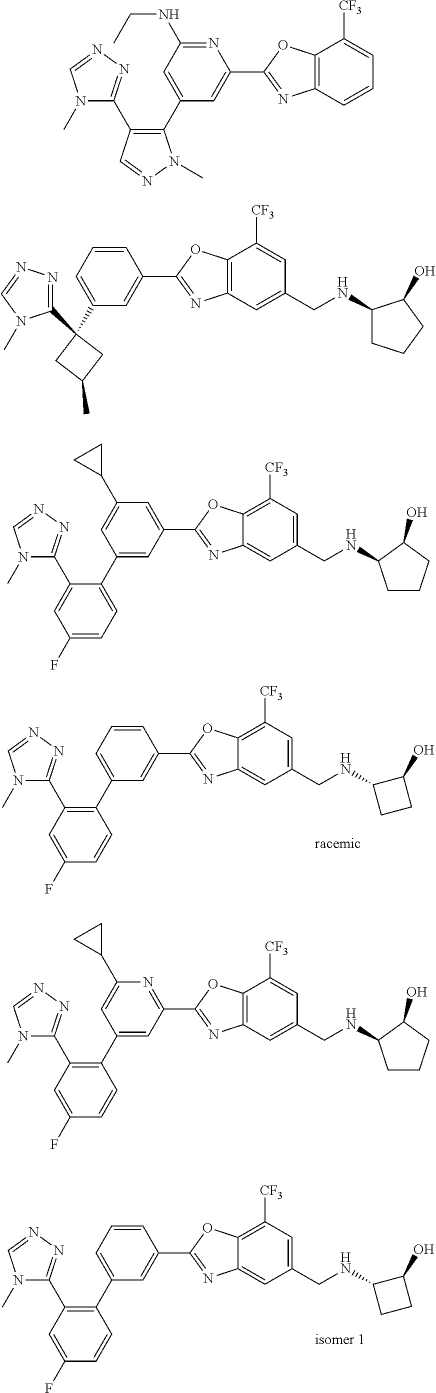 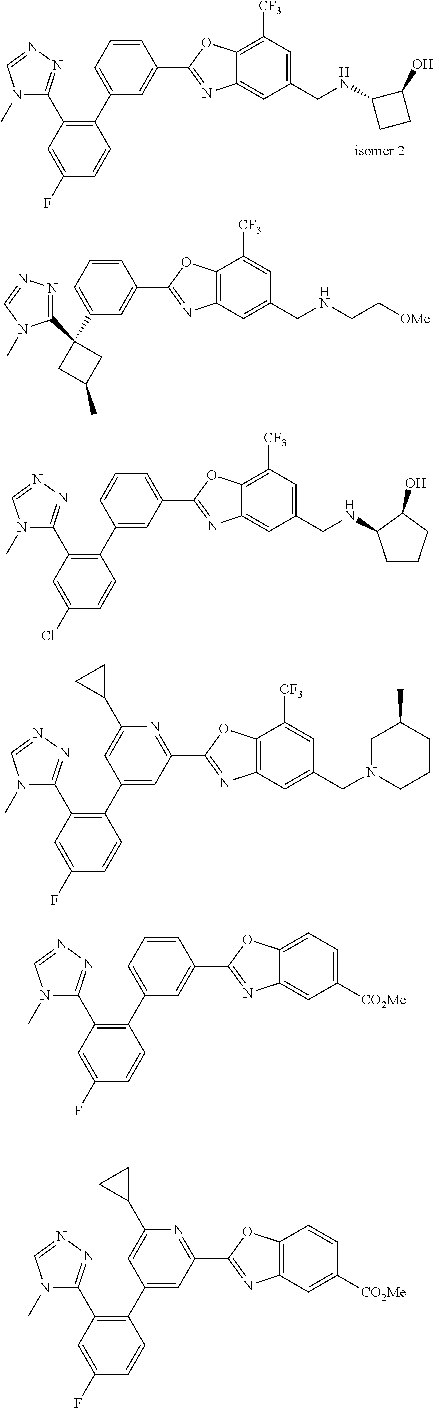 [SMILES: Cn1cnnc1-c1cc(F)ccc1-c1cccc(-c2nc3cc(CN[C@H]4CC[C@@H]4O)cc(C(F)(F)F)c3o2)c1 ; COCCNCc1cc(C(F)(F)F)c2oc(-c3cccc([C@]4(c5nncn5C)C[C@@H](C)C4)c3)nc2c1 ; Cn1cnnc1-c1cc(Cl)ccc1-c1cccc(-c2nc3cc(CN[C@@H]4CCC[C@@H]4O)cc(C(F)(F)F)c3o2)c1 ; C[C@H]1CCCN(Cc2cc(C(F)(F)F)c3oc(-c4cc(-c5ccc(F)cc5-c5nncn5C)cc(C5CC5)n4)nc3c2)C1 ; COC(=O)c1ccc2oc(-c3cccc(-c4ccc(F)cc4-c4nncn4C)c3)nc2c1 ; COC(=O)c1ccc2oc(-c3cc(-c4ccc(F)cc4-c4nncn4C)cc(C4CC4)n3)nc2c1] 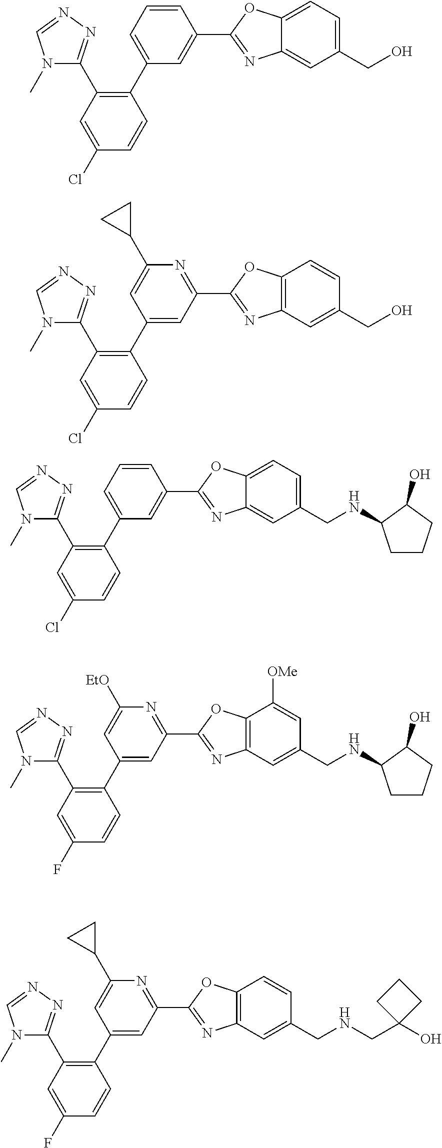 [SMILES: Cn1cnnc1-c1cc(Cl)ccc1-c1cccc(-c2nc3cc(CO)ccc3o2)c1 ; Cn1cnnc1-c1cc(Cl)ccc1-c1cc(-c2nc3cc(CO)ccc3o2)nc(C2CC2)c1 ; Cn1cnnc1-c1cc(Cl)ccc1-c1cccc(-c2nc3cc(CN[C@@H]4CCC[C@@H]4O)ccc3o2)c1 ; CCOc1cc(-c2ccc(F)cc2-c2nncn2C)cc(-c2nc3cc(CN[C@@H]4CCC[C@@H]4O)cc(OC)c3o2)n1 ; Cn1cnnc1-c1cc(F)ccc1-c1cc(-c2nc3cc(CNCC4(O)CCC4)ccc3o2)nc(C2CC2)c1] 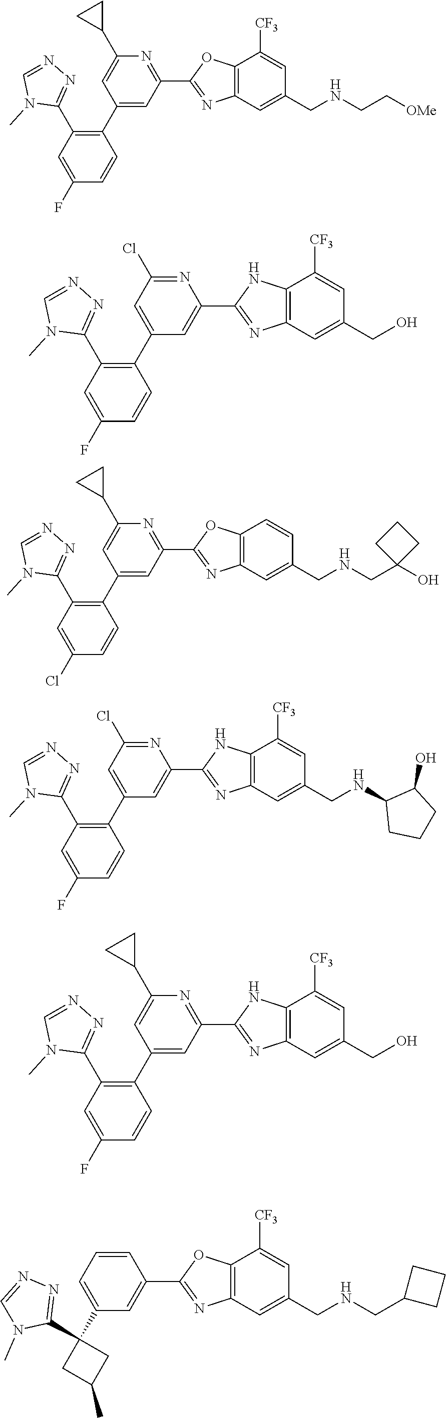 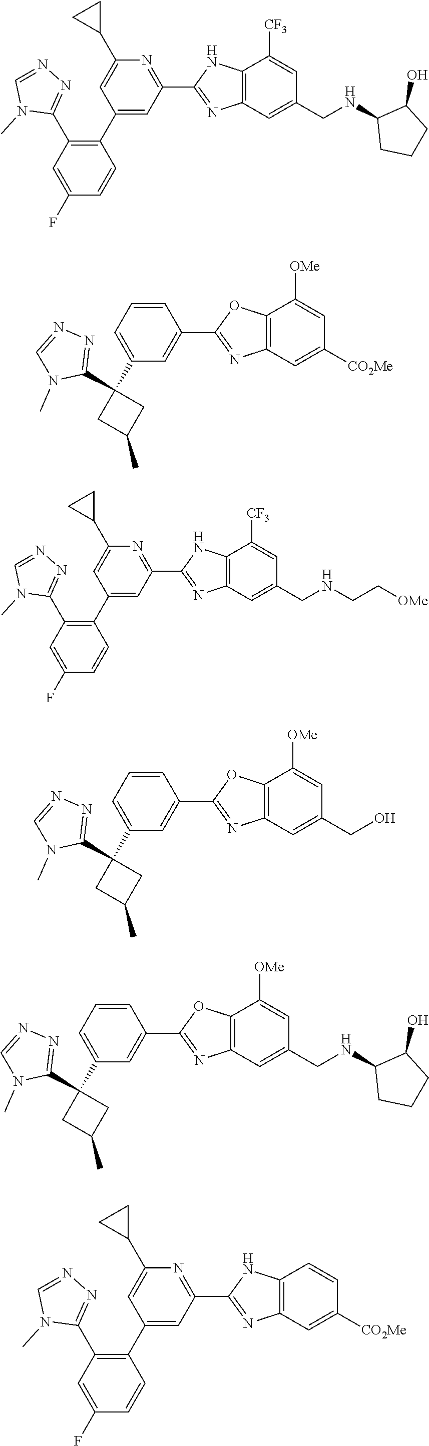 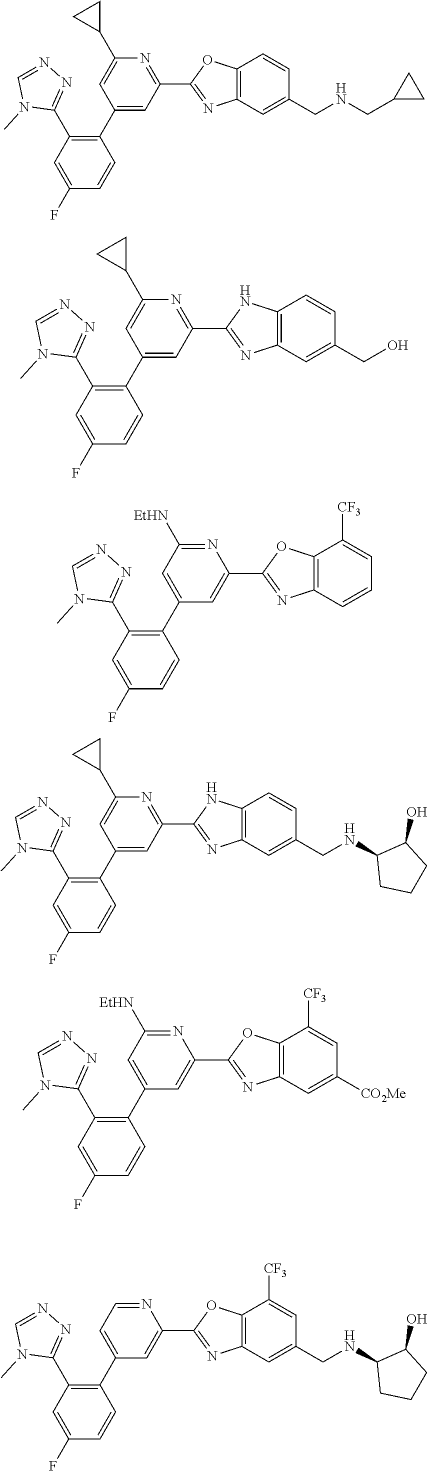 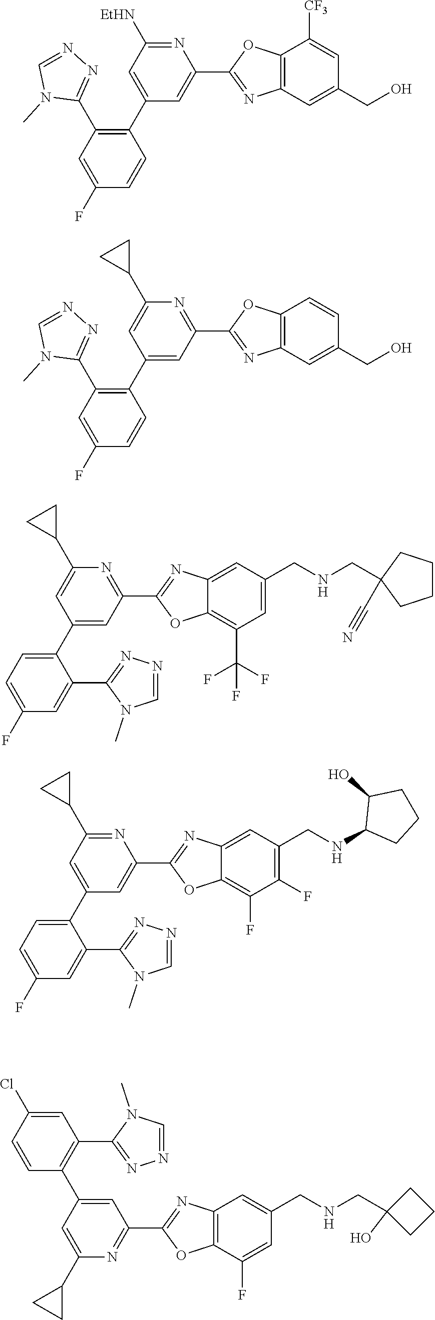 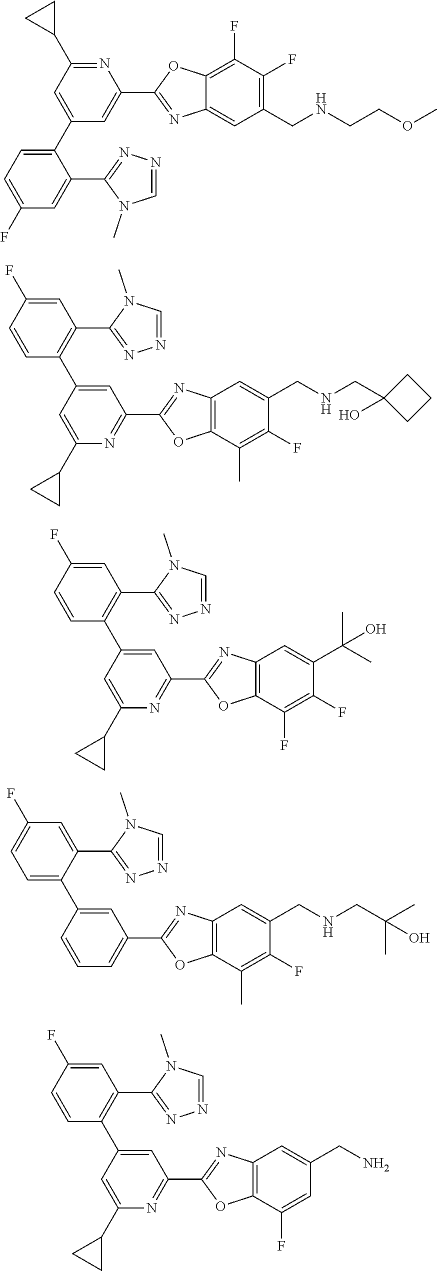 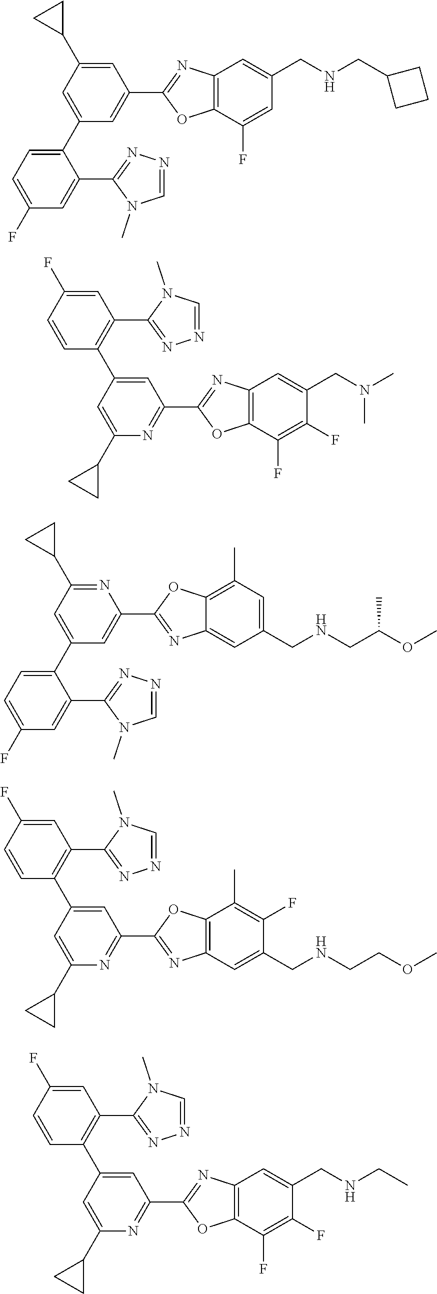 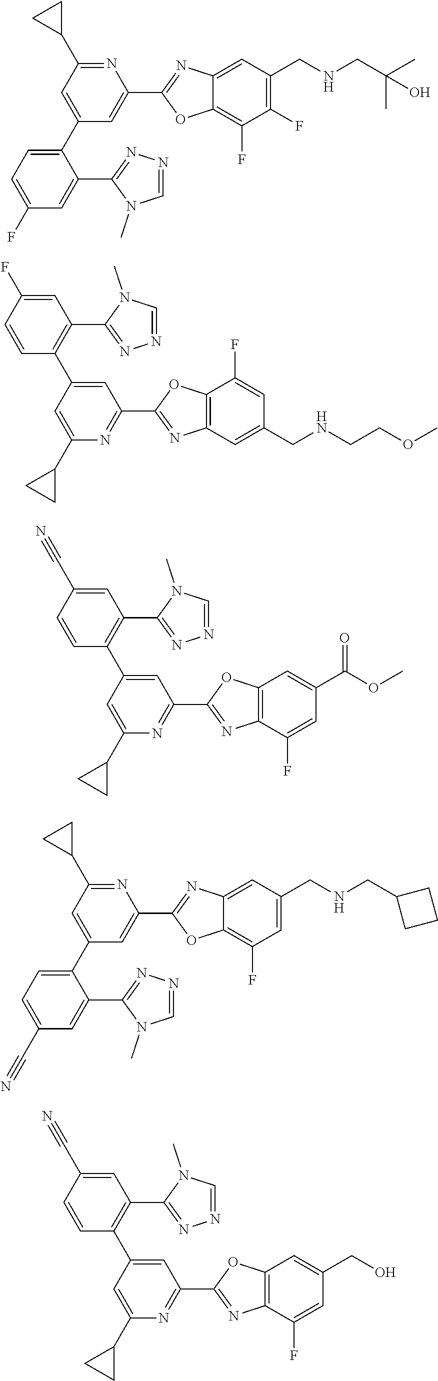 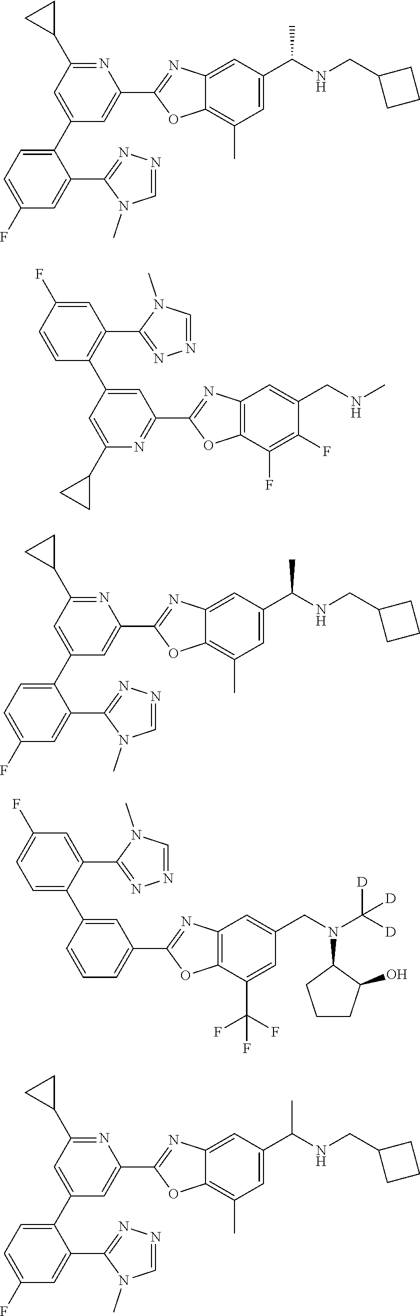 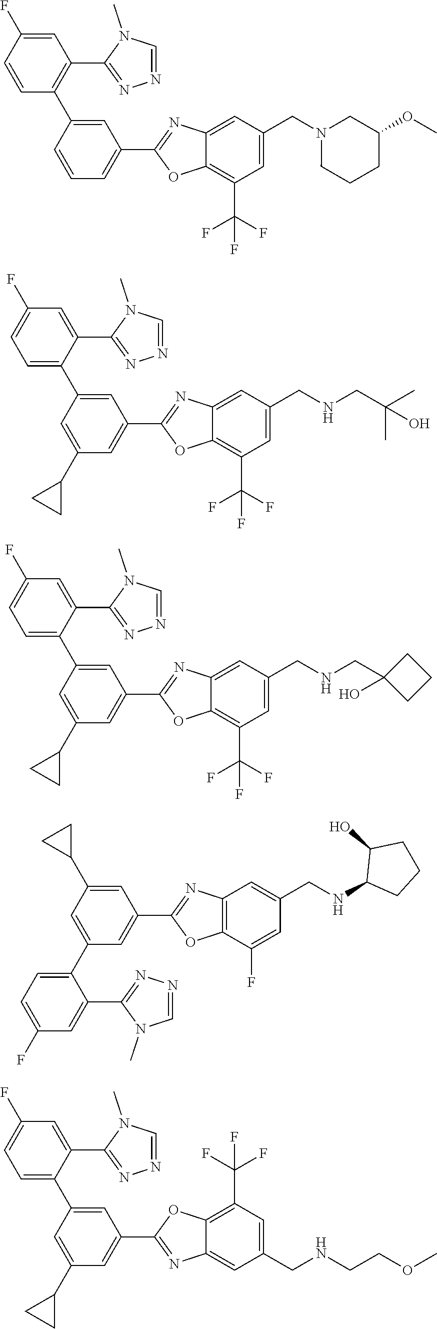 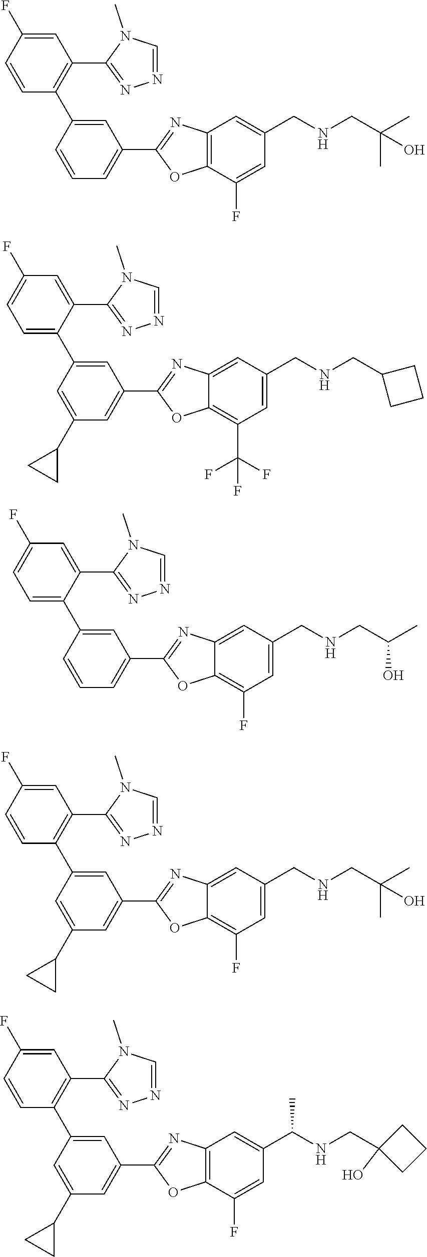 [SMILES: Cn1cnnc1-c1cc(F)ccc1-c1cccc(-c2nc3cc(CNCC(C)(C)O)cc(F)c3o2)c1 ; Cn1cnnc1-c1cc(F)ccc1-c1cc(-c2nc3cc(CNCC4CCC4)cc(C(F)(F)F)c3o2)cc(C2CC2)c1 ; C[C@H](O)CNCc1cc(F)c2oc(-c3cccc(-c4ccc(F)cc4-c4nncn4C)c3)nc2c1 ; Cn1cnnc1-c1cc(F)ccc1-c1cc(-c2nc3cc(CNCC(C)(C)O)cc(F)c3o2)cc(C2CC2)c1 ; C[C@H](NCC1(O)CCC1)c1cc(F)c2oc(-c3cc(-c4ccc(F)cc4-c4nncn4C)cc(C4CC4)c3)nc2c1] 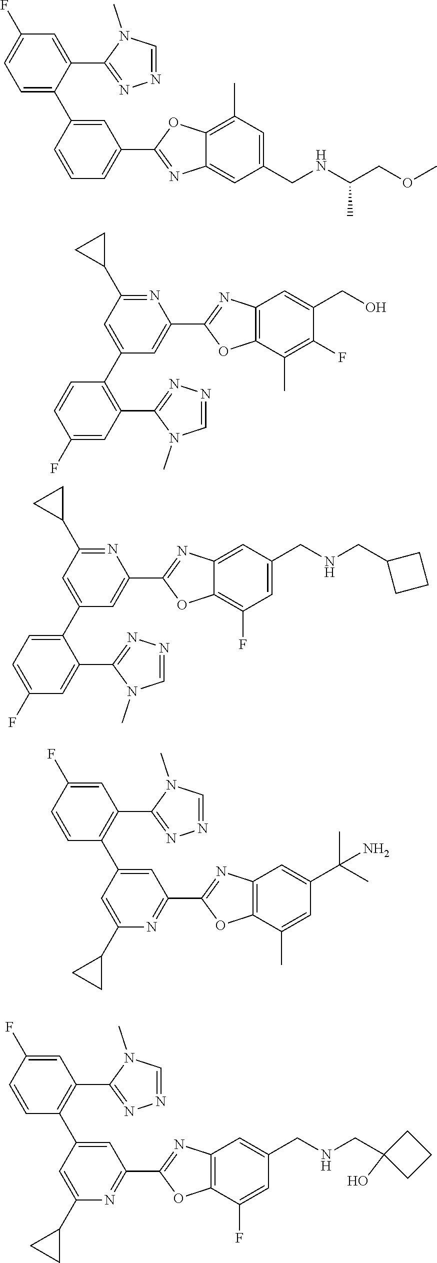 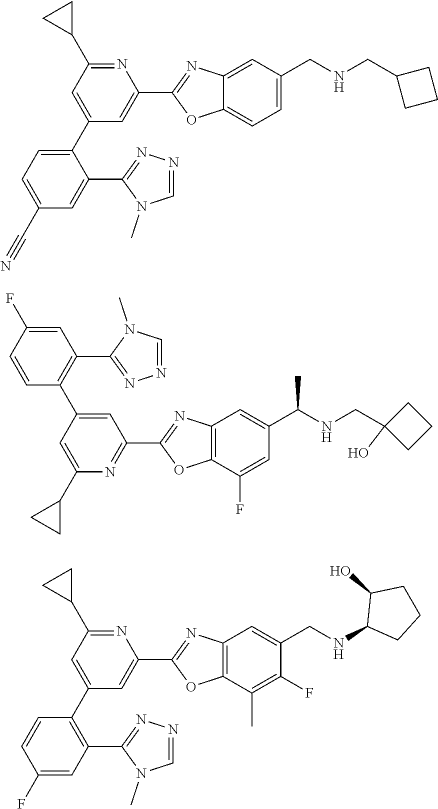 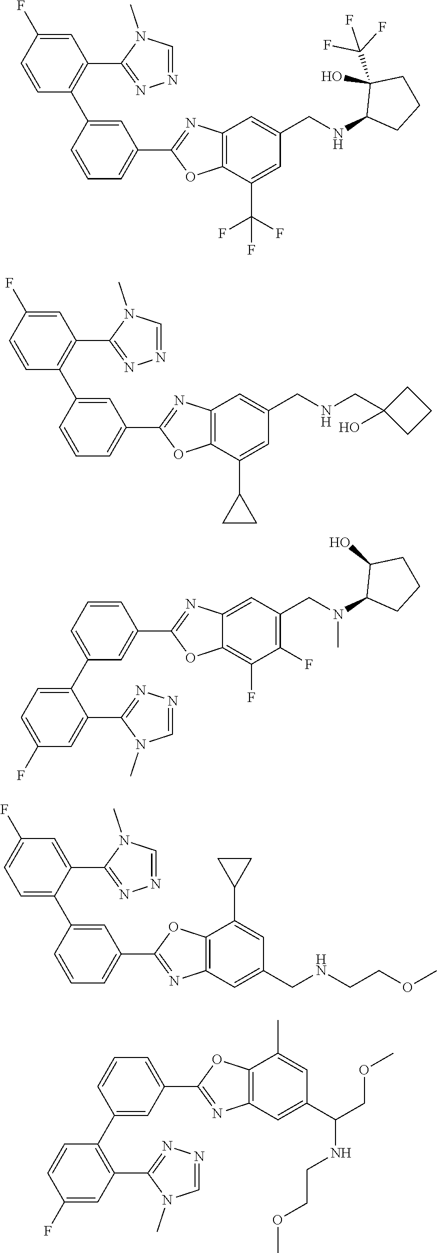 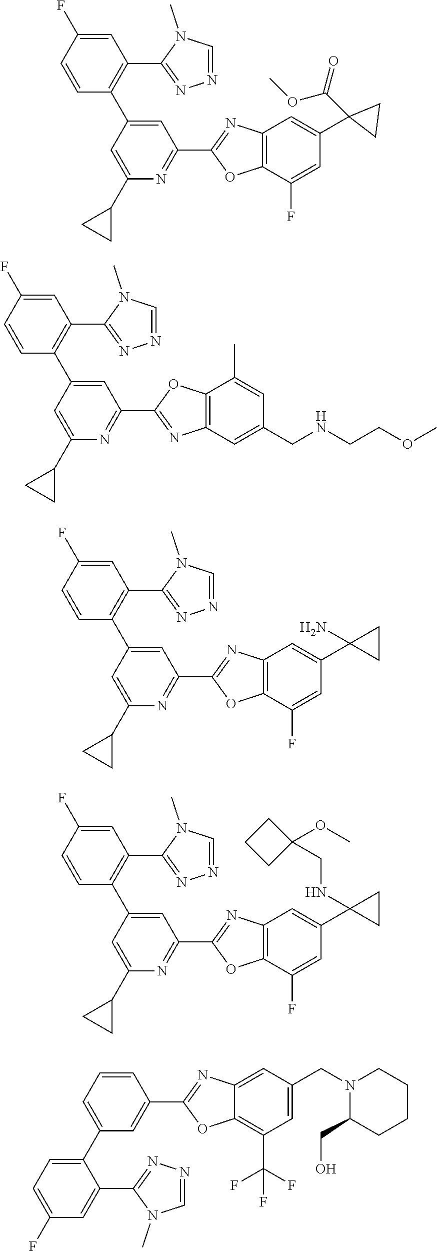 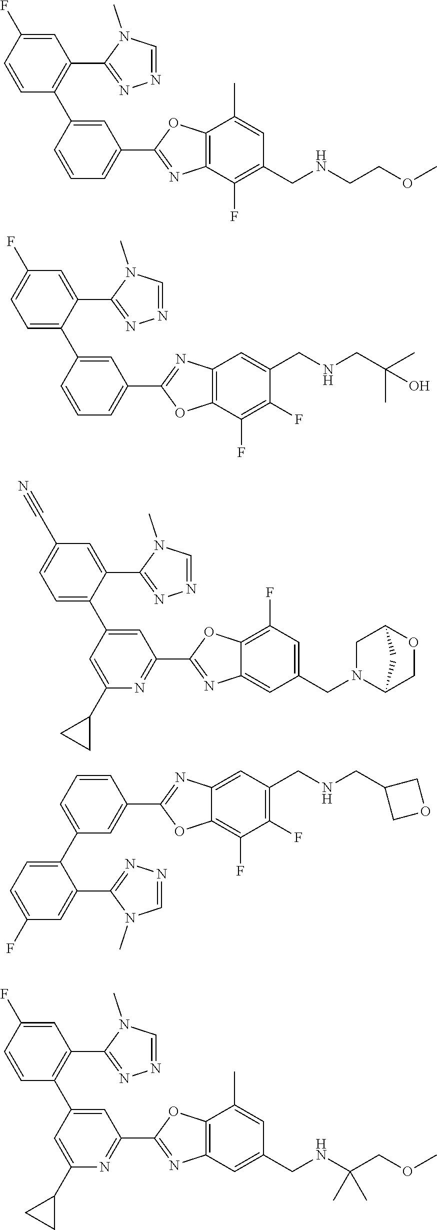 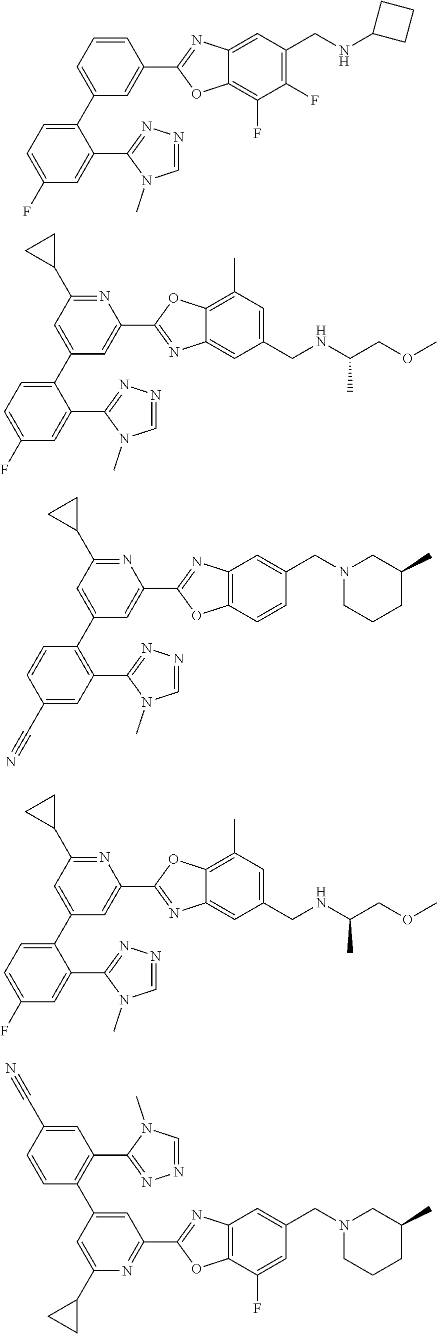 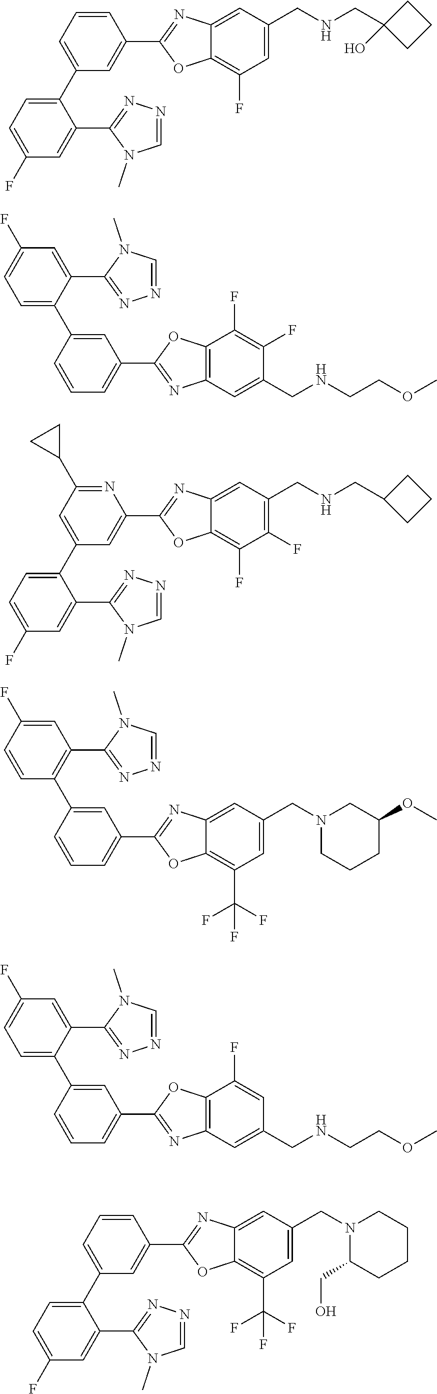 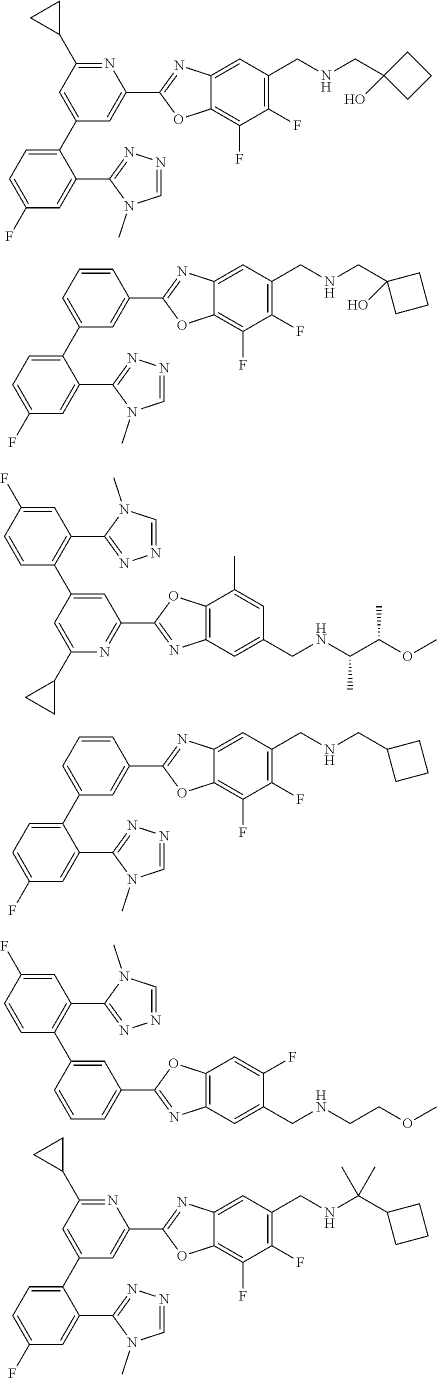 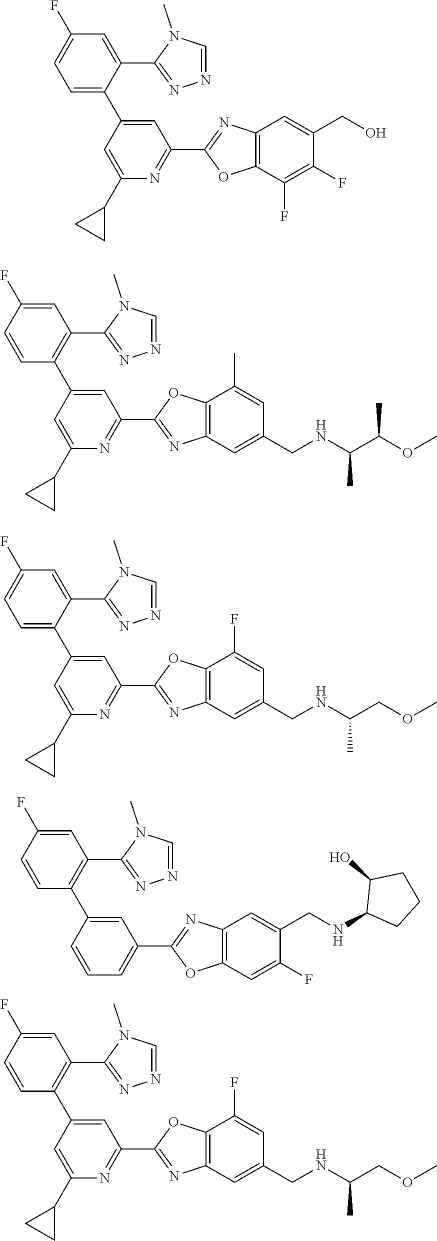 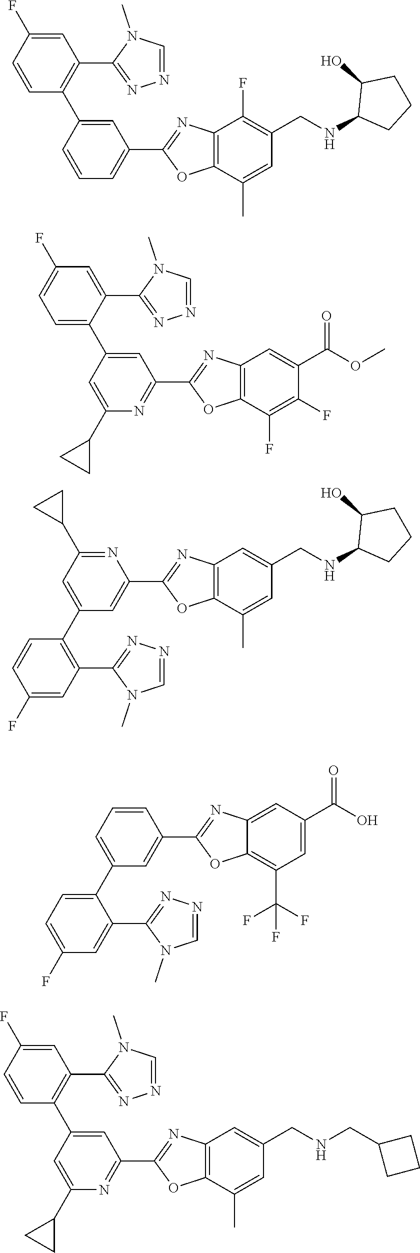 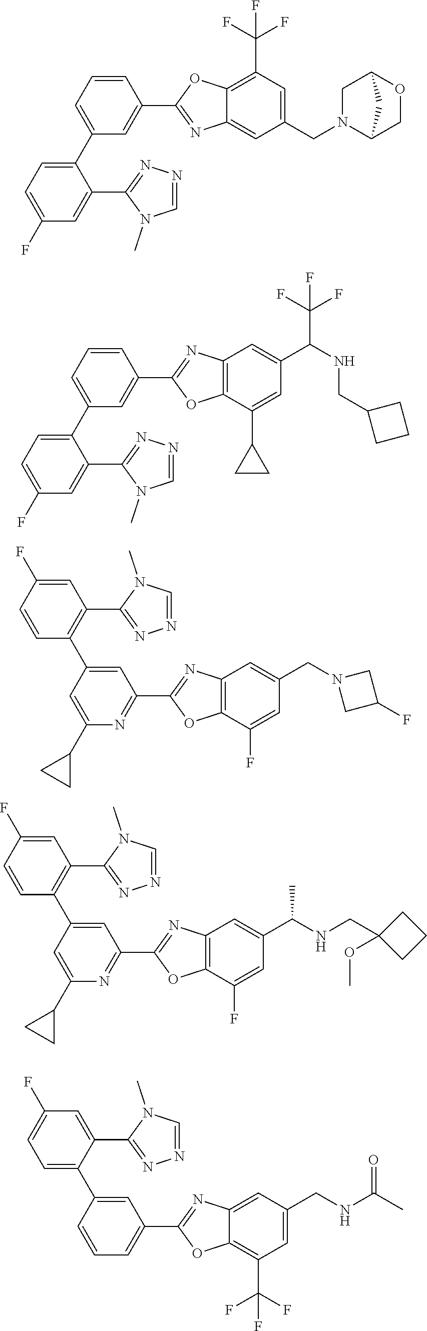 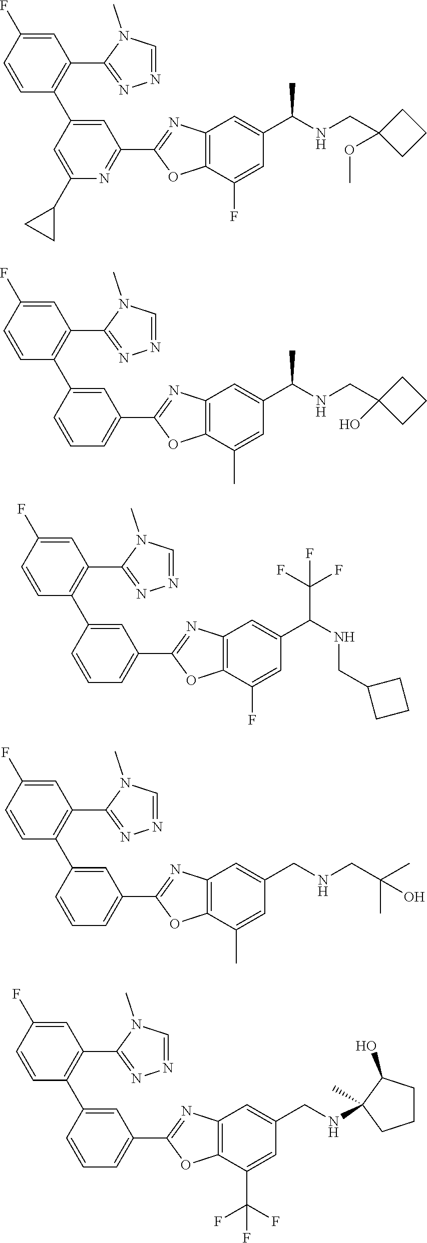 [SMILES: COC1(CN[C@H](C)c2cc(F)c3oc(-c4cc(-c5ccc(F)cc5-c5nncn5C)cc(C5CC5)n4)nc3c2)CCC1 ; Cc1cc([C@@H](C)NCC2(O)CCC2)cc2nc(-c3cccc(-c4ccc(F)cc4-c4nncn4C)c3)oc12 ; Cn1cnnc1-c1cc(F)ccc1-c1cccc(-c2nc3cc(C(NCC4CCC4)C(F)(F)F)cc(F)c3o2)c1 ; Cc1cc(CNCC(C)(C)O)cc2nc(-c3cccc(-c4ccc(F)cc4-c4nncn4C)c3)oc12 ; Cn1cnnc1-c1cc(F)ccc1-c1cccc(-c2nc3cc(CN[C@]4(C)CCC[C@@H]4O)cc(C(F)(F)F)c3o2)c1] 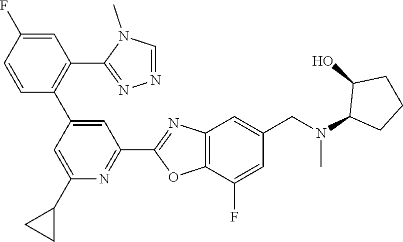 [SMILES: CN(Cc1cc(F)c2oc(-c3cc(-c4ccc(F)cc4-c4nncn4C)cc(C4CC4)n3)nc2c1)[C@@H]1CCC[C@@H]1O] 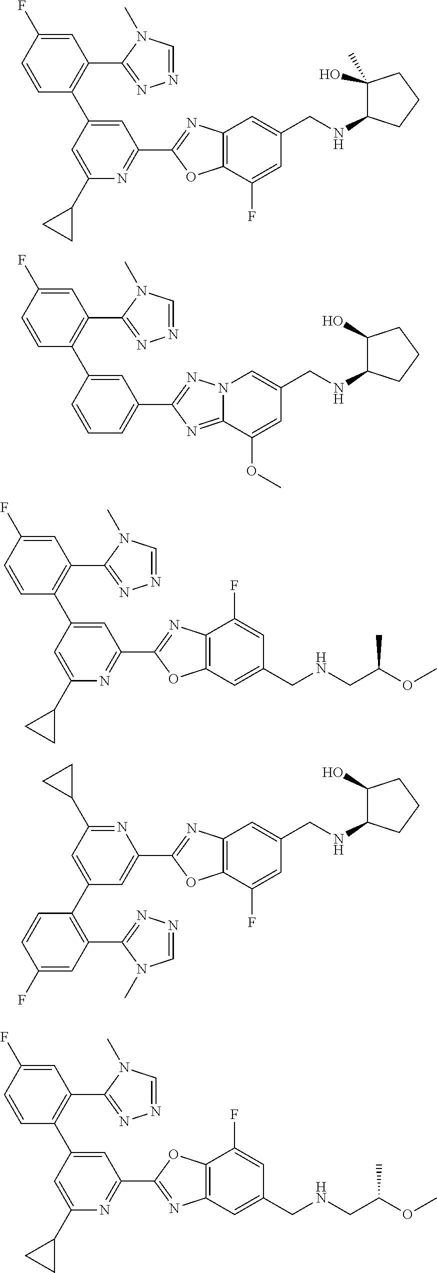 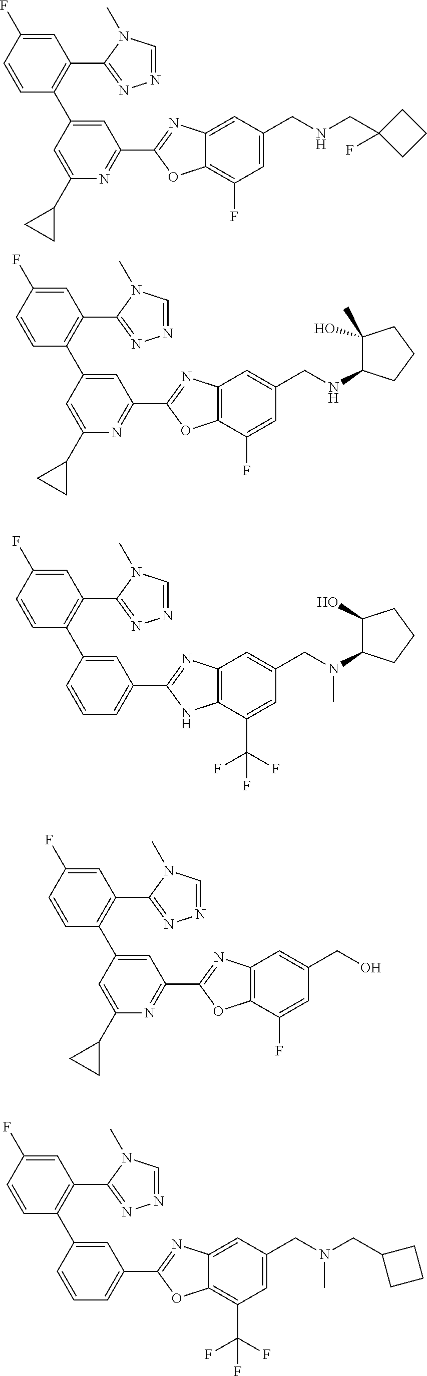 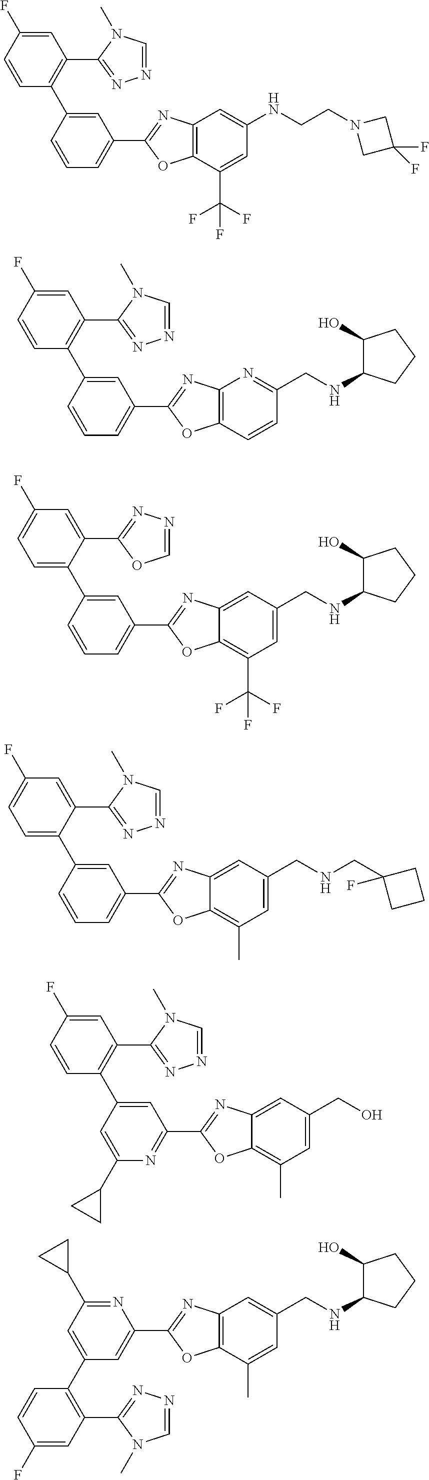 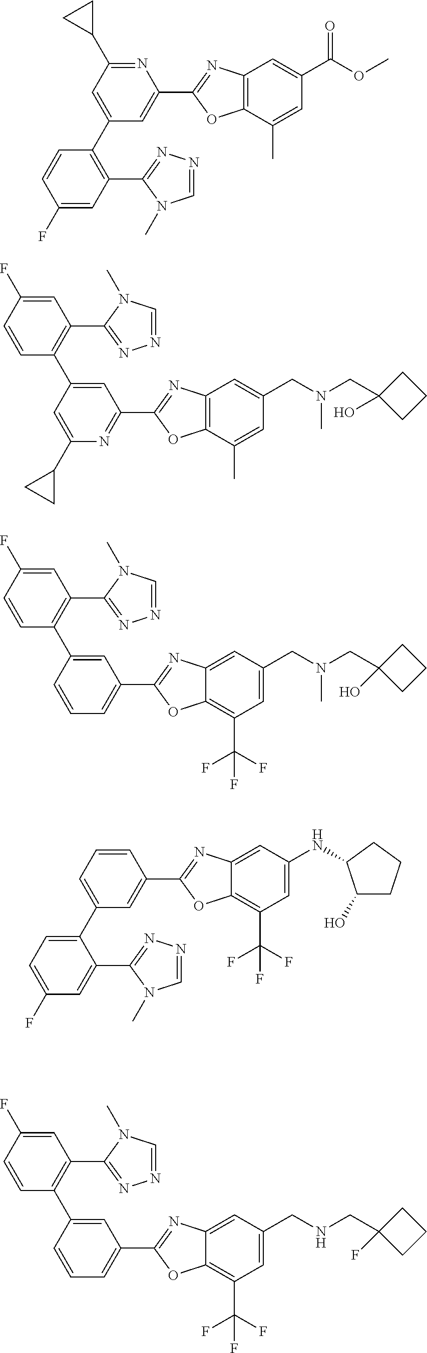 [SMILES: COC(=O)c1cc(C)c2oc(-c3cc(-c4ccc(F)cc4-c4nncn4C)cc(C4CC4)n3)nc2c1 ; Cc1cc(CN(C)CC2(O)CCC2)cc2nc(-c3cc(-c4ccc(F)cc4-c4nncn4C)cc(C4CC4)n3)oc12 ; CN(Cc1cc(C(F)(F)F)c2oc(-c3cccc(-c4ccc(F)cc4-c4nncn4C)c3)nc2c1)CC1(O)CCC1 ; Cn1cnnc1-c1cc(F)ccc1-c1cccc(-c2nc3cc(N[C@@H]4CCC[C@@H]4O)cc(C(F)(F)F)c3o2)c1 ; Cn1cnnc1-c1cc(F)ccc1-c1cccc(-c2nc3cc(CNCC4(F)CCC4)cc(C(F)(F)F)c3o2)c1] 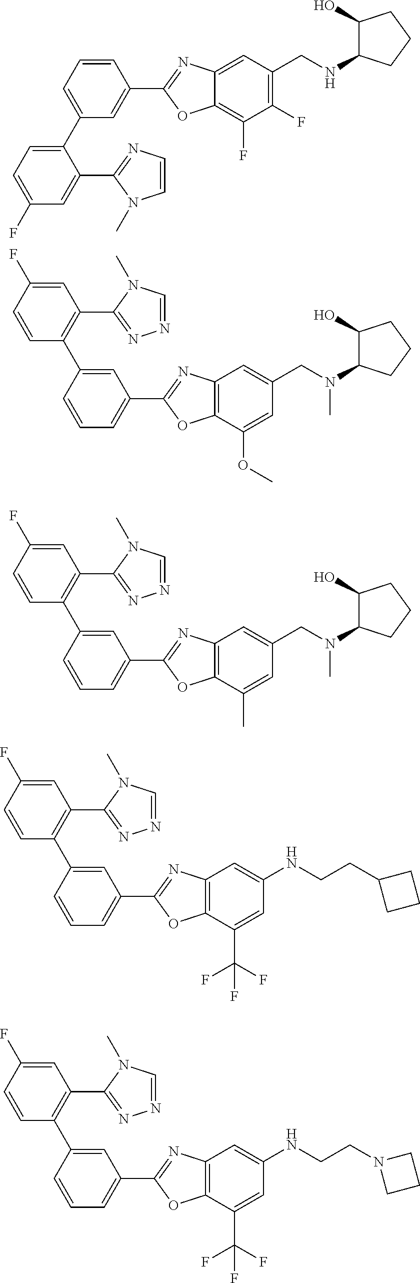 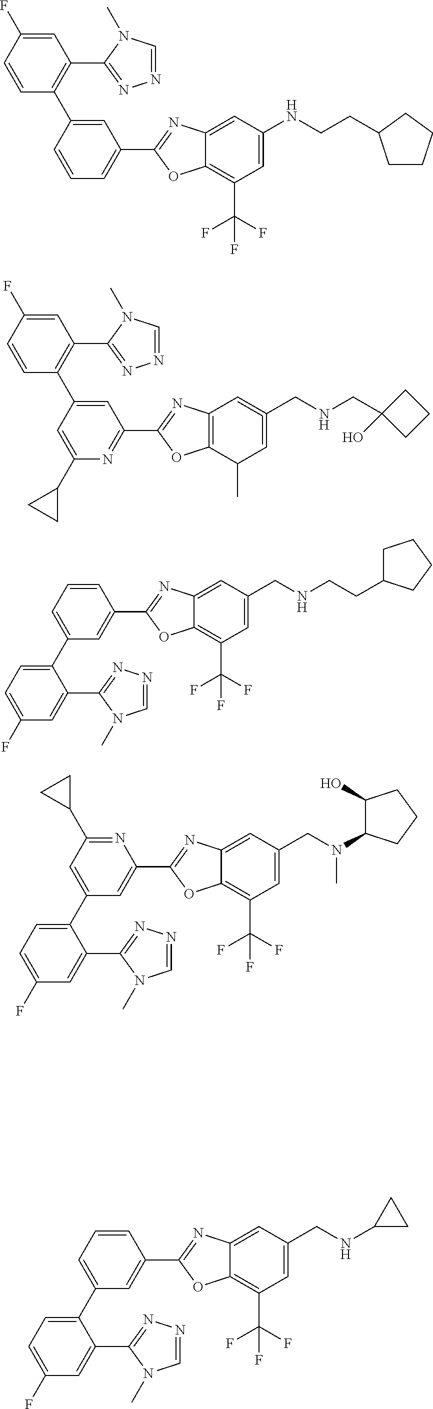 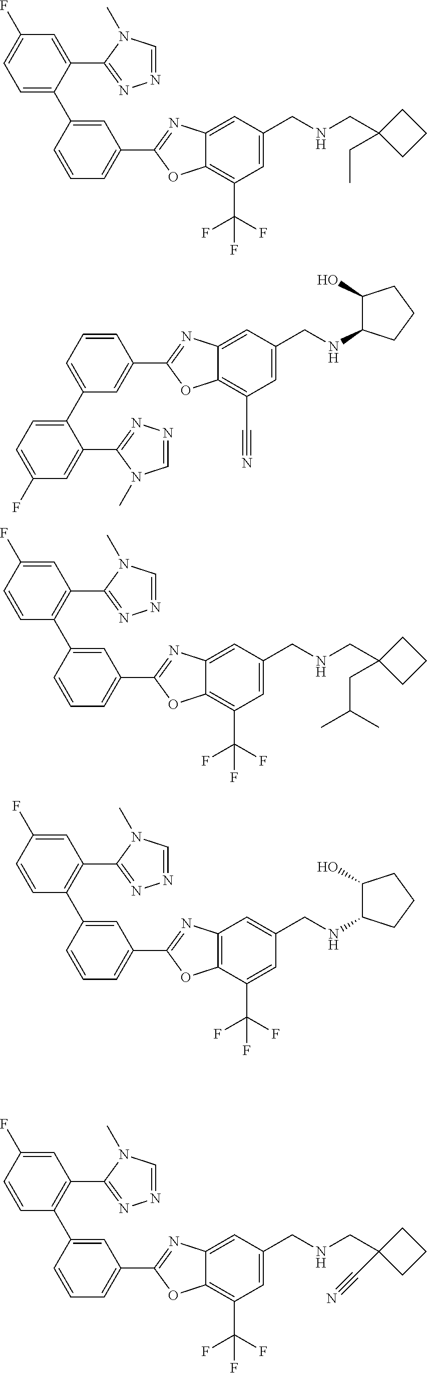 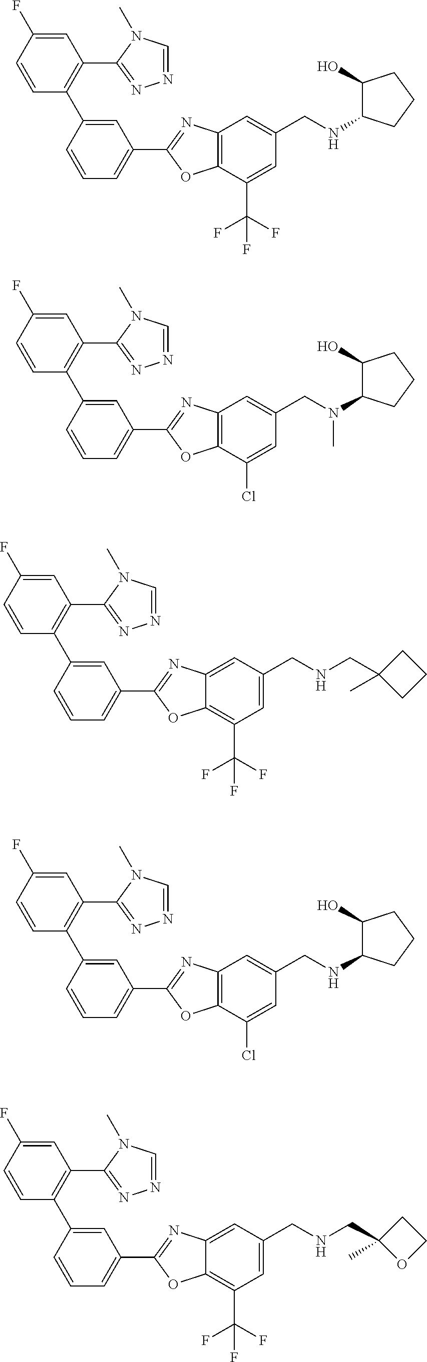 [SMILES: Cn1cnnc1-c1cc(F)ccc1-c1cccc(-c2nc3cc(CN[C@H]4CCC[C@@H]4O)cc(C(F)(F)F)c3o2)c1 ; CN(Cc1cc(Cl)c2oc(-c3cccc(-c4ccc(F)cc4-c4nncn4C)c3)nc2c1)[C@@H]1CCC[C@@H]1O ; Cn1cnnc1-c1cc(F)ccc1-c1cccc(-c2nc3cc(CNCC4(C)CCC4)cc(C(F)(F)F)c3o2)c1 ; Cn1cnnc1-c1cc(F)ccc1-c1cccc(-c2nc3cc(CN[C@@H]4CCC[C@@H]4O)cc(Cl)c3o2)c1 ; Cn1cnnc1-c1cc(F)ccc1-c1cccc(-c2nc3cc(CNC[C@@]4(C)CCO4)cc(C(F)(F)F)c3o2)c1] 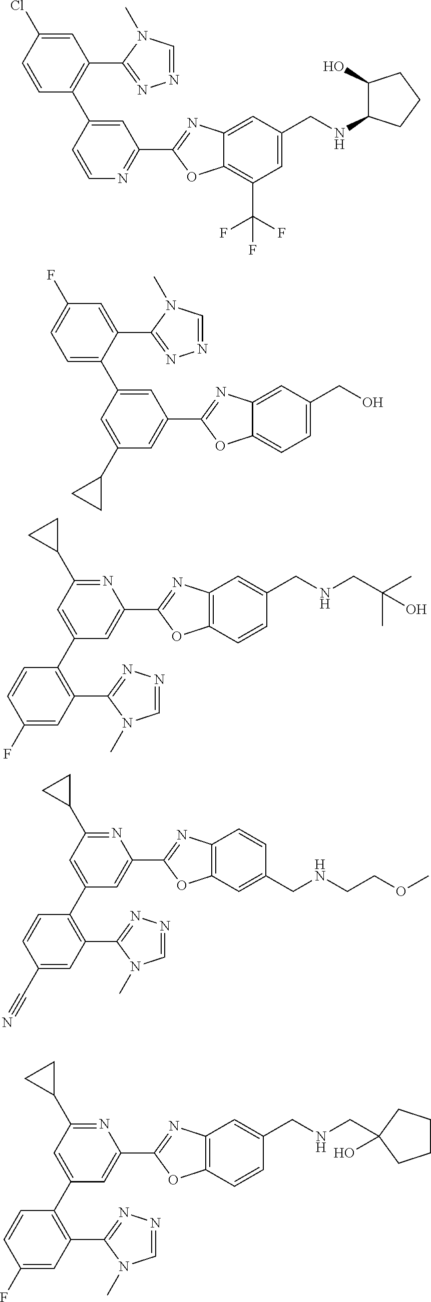 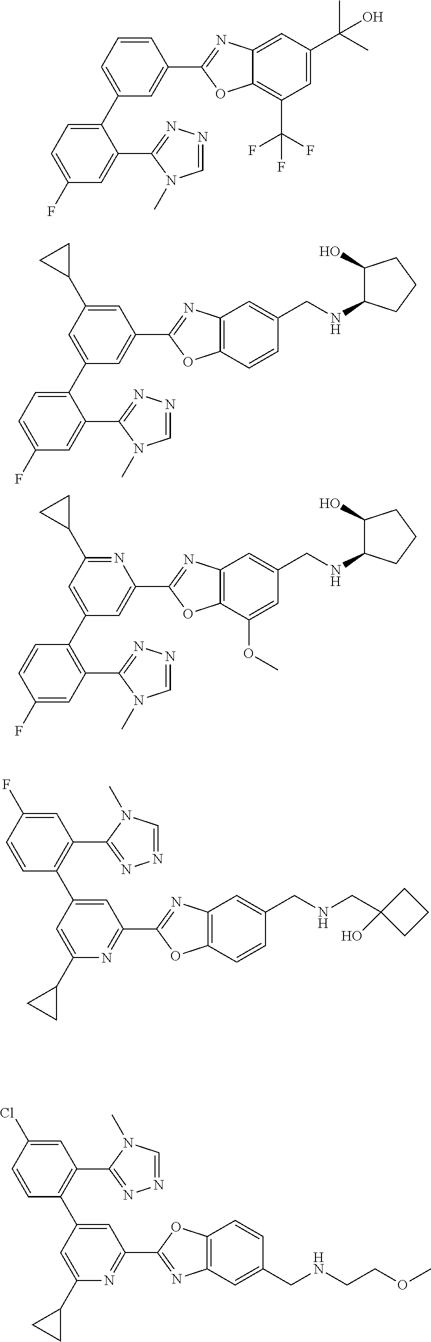 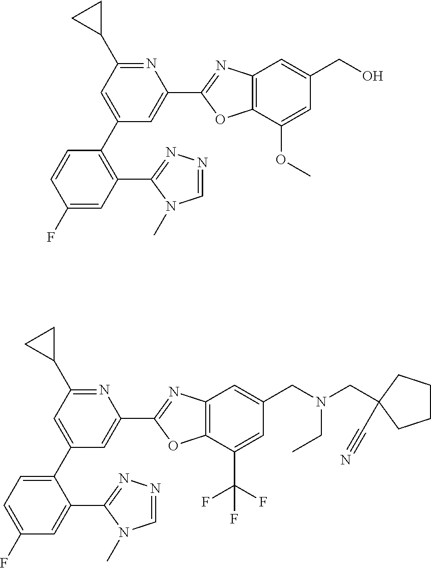 [SMILES: COc1cc(CO)cc2nc(-c3cc(-c4ccc(F)cc4-c4nncn4C)cc(C4CC4)n3)oc12 ; CCN(Cc1cc(C(F)(F)F)c2oc(-c3cc(-c4ccc(F)cc4-c4nncn4C)cc(C4CC4)n3)nc2c1)CC1(C#N)CCCC1]
or a pharmaceutically acceptable salt thereof.

23. A pharmaceutical composition comprising a compound according to claim 1, or a pharmaceutically acceptable salt thereof, and a pharmaceutically acceptable carrier, adjuvant, or vehicle.

24. (canceled)

25. A method of inhibiting Cbl-b in a biological sample comprising contacting the sample with the compound of claim 1, or a pharmaceutically acceptable salt thereof, or the pharmaceutical composition thereof.

26. A method of treating a Cbl-b-mediated disorder, disease, or condition in a patient comprising administering to said patient the compound of claim 1, or a pharmaceutically acceptable salt thereof, or the pharmaceutical composition of claim 23.

27-29. (canceled)
